(19) United States
(12) Patent Application Publication
(10) Pub. No.: US 2025/0340552 A1
(43) Pub. Date: Nov. 6, 2025

(54) COMPOUNDS, COMPOSITIONS AND METHODS OF TREATING DISORDERS

(71) Applicant: HOTSPOT THERAPEUTICS, INC., Boston, MA (US)

(72) Inventors: Yingzhi BI, Plainsboro, NJ (US); Kenneth G. CARSON, Princeton, NJ (US); Brian David HAMMAN, Monroe Township, NJ (US); Geraldine Cirillo HARRIMAN, Charlestown, RI (US); Graham A.B. HONE, Hopewell, NJ (US); Rajiv Gandhi GOVINDARAJ, Mansfield, MA (US)

(21) Appl. No.: 19/172,052

(22) Filed: Apr. 7, 2025

Related U.S. Application Data
(63) Continuation of application No. PCT/US2023/076248, filed on Oct. 6, 2023.
(60) Provisional application No. 63/414,343, filed on Oct. 7, 2022.

(51) Int. Cl. C07D 471/04; A61K 31/4545; A61K 31/496; A61K 31/4995; A61K 31/55; A61K 31/551; C07D 401/14; C07D 405/14; C07D 519/00

(52) U.S. Cl. CPC C07D 471/04; A61K 31/4545; A61K 31/496; A61K 31/4995; A61K 31/55; A61K 31/551; C07D 401/14; C07D 405/14; C07D 519/00

(57) ABSTRACT

The present disclose includes, among other things, compounds that treat or lessen the severity of cancer, pharmaceutical compositions and methods of making and using the same.

### CROSS REFERENCE TO RELATED APPLICATIONS

This application is a continuation of International (PCT) Patent Application No. PCT/US2023/076248, filed Oct. 6, 2023, which claims priority to U.S. Provisional Application 63/414,343 filed Oct. 7, 2022, the contents of each of which are incorporated herein by reference.

### BACKGROUND

Cbl-b is a E3 ubiquitin-protein ligase that functions as a negative regulator of T-cell activation. Modulation of Cbl-b has been shown to be a therapeutic target for a diseases and disorders. There remains a need for compounds that inhibit Cbl-b.

### SUMMARY

In some embodiments, the present disclosure includes a compound of formula (A-1) or (A-2):

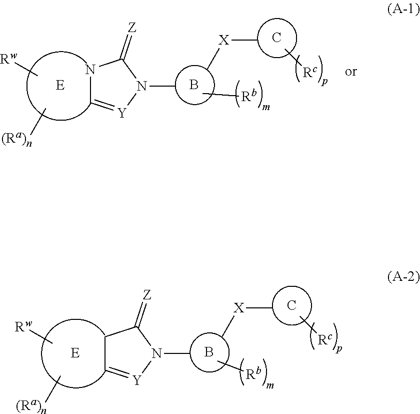

or a pharmaceutically acceptable salt thereof.

Additionally, the present disclosure includes, among other things, pharmaceutical compositions, methods of using and methods of making a compound of formula (A-1) or (A-2).

### DETAILED DESCRIPTION

In some embodiments, the present disclosure includes a compound of formula (A-1) or (A-2):

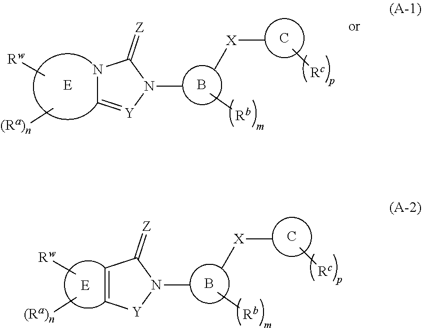

or pharmaceutically acceptable salts thereof,
   wherein
Y is selected from the group ═C(H)—, ═C(R^{a})— or ═N—;
Z is ═O or ═S;
E is optionally substituted 5-6 membered heterocyclyl or optionally substituted phenyl;
B is optionally substituted phenyl, optionally substituted 8-10 membered bicyclyl, or optionally substituted 5-6 membered heteroaryl;
C is optionally substituted 5-6 membered heteroaryl;
X is an optionally substituted C₁-C₃ alkylene chain, wherein one or more methylene units is optionally and independently replaced by —N(H)—, —N(R¹)—, —O—, —S—, —SO—, —SO₂—, optionally substituted 3-6-membered carbocyclyl, and optionally substituted 3-6-membered heterocyclyl, wherein X is optionally substituted with an optionally substituted group selected from the group consisting of halogen, C₁-C₃ aliphatic, phenyl, 3-6-membered heteroaryl, 3-6-membered heterocyclyl, and —(CH₂)(3-6-membered carbocyclyl);
each R^{a} is independently selected from the group consisting of halogen, —CN, —OH, —OR¹, —NH₂, —NR¹R², —SH, —SR¹, —SF₅, —CO₂H, —CO₂R¹, —C(O)R¹, —CONH₂, —CONR¹R², —SO₂NH₂, —SO₂NR¹R², —SO₂OH, —SO₂OR¹, —S(O)R¹, —S(O)₂R¹, —S(O)(NH)R¹, —S(O)(NR¹)R¹, optionally substituted C₁-C₆ aliphatic, optionally substituted C₁-C₆ heteroalkyl, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S, wherein R^{a} is optionally substituted with 1-5 instances of R^{a1};
each R^{a1} is independently selected from the group consisting of halogen, —CN, —OH, —OR¹, —NH₂, —NR¹R², —SH, —SR¹, —SF₅, —CO₂H, —CO₂R¹, —CONH₂, —CONR¹R², —SO₂NH₂, —SO₂NR¹R², —SO₂OH, —SO₂OR¹, —S(O)R¹, —S(O)₂R¹, —S(O)(NH)R¹, —S(O)(NR¹)R¹, optionally substituted C₁-C₆ aliphatic, optionally substituted C₁-C₆ heteroalkyl, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S;
R^{w} is -L¹-A-L²-G;
L¹ is a bond or an optionally substituted C₁-C₃ alkylene chain;
L² is an optionally substituted C₁-C₁₂ alkylene chain, wherein 1-6 methylene units of L² are optionally and independently replaced by —C(O)—, —N(H)—, —N(R¹)—, —O—, —S—, —SO—, —SO₂—, optionally substituted 3-6-membered carbocyclyl, and optionally substituted 3-6-membered heterocyclyl, wherein X is optionally substituted with an optionally substituted group selected from the group consisting of halogen, C₁-C₃ aliphatic, phenyl, 3-6-membered heteroaryl, and 3-6-membered heterocyclyl;
A is a bivalent group selected from the group consisting of a bond, optionally substituted C₃-C₇ carbocyclylene, optionally substituted C₁-C₆ heteroalkylene, optionally substituted 3-6 membered heterocyclylene containing 1-4 heteroatoms each selected from the group consisting of N, O, and S, optionally substituted phenylene, and optionally substituted 5-6-membered heteroarylene containing 1-4 heteroatoms each selected from the group consisting of N, O and S, wherein A is optionally substituted with 1-5 instances of R^{a1};
G is selected from the group consisting of optionally substituted C₃-C₁₀ carbocylyl, optionally substituted C₁-C₆ heteroalkyl, optionally substituted 3-10 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S, wherein G is optionally substituted with 1-5 instances of R^{g1};
each R^{g1} is independently selected from the group consisting of halogen, —CN, —OH, —OR¹, —NH₂, —NR¹R², —SH, —SR¹, —SF₅, —CO₂H, —CO₂R¹, —CONH₂, —CONR¹R², —SO₂NH₂, —SO₂NR¹R², —SO₂OH, —SO₂OR¹, —S(O)R¹, —S(O)₂R¹, —S(O)(NH)R¹, —S(O)(NR¹)R¹, optionally substituted C₁-C₆ aliphatic, optionally substituted C₁-C₆ heteroalkyl, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S;
each R^{b} is independently selected from the group consisting of, halogen, —CN, —OH, —OR¹, —NH₂, —NR¹R², —SH, —SR¹, —SF₅, —CO₂H, —CO₂R¹, —CONH₂, —CONR¹R², —SO₂NH₂, —SO₂NR¹R², —SO₂OH, —SO₂OR¹, —S(O)R¹, —S(O)₂R¹, —S(O)(NH)R¹, —S(O)(NR¹)R¹, optionally substituted C₁-C₆ aliphatic, optionally substituted C₁-C₆ heteroalkyl, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S;
each R^{c} is independently selected from the group consisting of hydrogen, optionally substituted C₁-C₆ aliphatic, OR¹, —NH₂, —NR¹R², optionally substituted phenyl, optionally substituted 3-6 membered heterocyclyl containing 1-4 heteroatoms each selected from the group consisting of N, O, and S, optionally substituted 5-6-membered heteroaryl containing 1-4 heteroatoms each selected from the group consisting of N, O and S, —C(O)R³, —CO₂R³, —C(O)NHR³, and —SO₂R³;
each R¹ is independently selected from the group consisting of optionally substituted C₁-C₆ aliphatic, optionally substituted phenyl, optionally substituted 3-6 membered heterocyclyl containing 1-4 heteroatoms each selected from the group consisting of N, O, and S, optionally substituted 5-6-membered heteroaryl containing 1-4 heteroatoms each selected from the group consisting of N, O and S, —C(O)R³, —CO₂R³, —C(O)NHR³, and —SO₂R³;
each R² is independently selected from the group consisting of hydrogen, optionally substituted C₁-C₆ aliphatic, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S;
   or R¹ and R² are taken together with their intervening atom(s) to form a 3-8-membered heterocyclyl ring containing 1-3 heteroatoms selected from the group consisting of N, O, and S, or an optionally substituted 5-6-membered heteroaryl ring containing 1-4 heteroatoms selected from the group consisting of N, O, and S.
each R³ is independently selected from the group consisting of optionally substituted C₁-C₆ aliphatic, optionally substituted 3-6 membered heterocyclyl containing 1-4 heteroatoms each selected from the group consisting of N, O, and S, optionally substituted phenyl, optionally substituted 5-6-membered heteroaryl containing 1-4 heteroatoms each selected from the group consisting of N, O and S;
n is 0, 1, 2, 3, 4, or 5;
m is 0, 1, 2, 3, or 4; and
p is 0, 1, 2, 3, or 4.

In some embodiments, the present disclosure includes a compound of Formula (B-1) or (B-2):

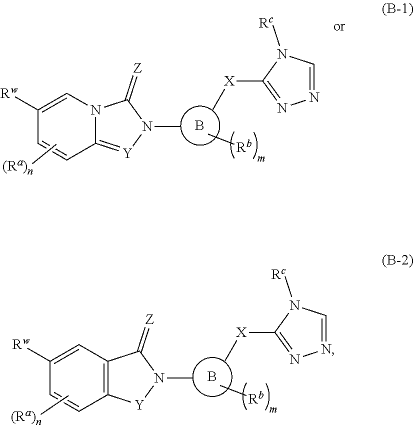

or pharmaceutically acceptable salts thereof,
   wherein B, X, Y, Z, R^{a}, R^{b}, R^{c}, R^{w}, n, and m are defined herein.

In some embodiments, the present disclosure includes a compound of formula (I-1 or I-2):

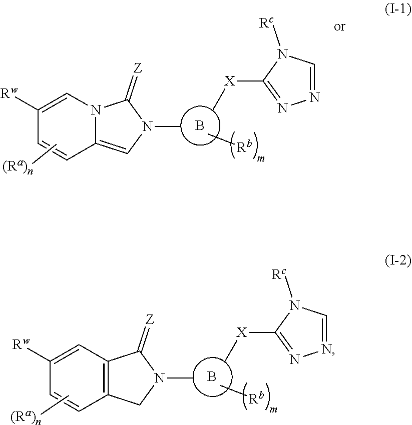

or pharmaceutically acceptable salts thereof,
   wherein B, X, R^{a}, R^{b}, R^{c}, R^{w}, n, and m are defined herein.

In some embodiments, present disclosure includes a compound is of formula (Ia) or (IIa):

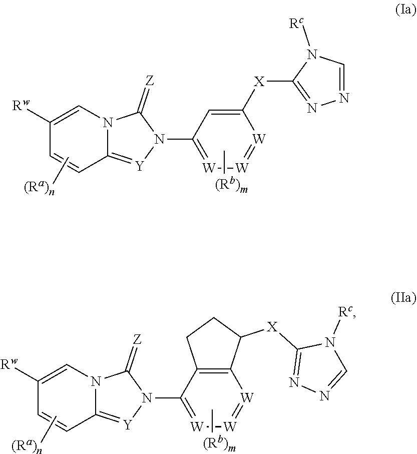

or pharmaceutically acceptable salts thereof,
wherein each W is independently selected from N or C; and
X, Y, Z, R^{a}, R^{b}, R^{c}, R^{w}, n, and m are defined above and described in classes and subclasses herein.

In some embodiments, present disclosure includes a compound is of formula (Ia1), (IIa1), (Ia1′), or (IIa1′):

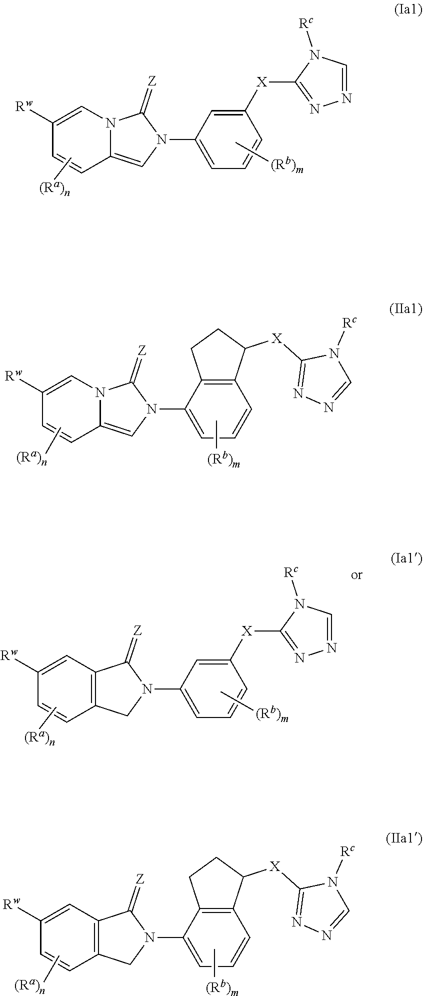

or a pharmaceutically acceptable salt thereof, wherein X, Y, Z, R^{a}, R^{b}, R^{c}, R^{w}, n, and m are defined above and described in classes and subclasses herein.

In some embodiments, present disclosure includes a compound is of formula (Ia2), (Ia3), or (Ia4):

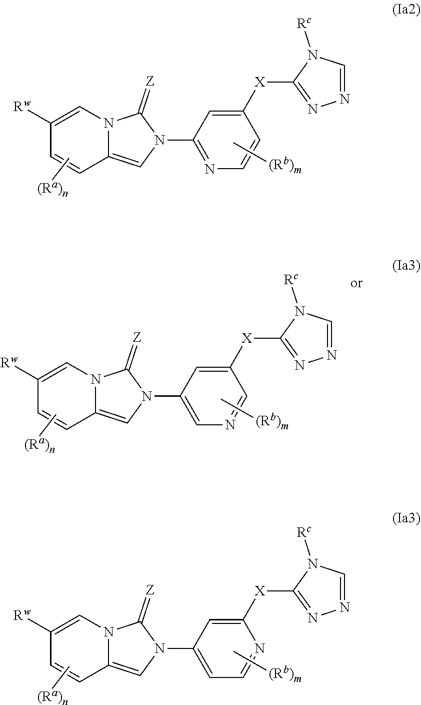

or pharmaceutically acceptable salts thereof, wherein X, Y, Z, R^{a}, R^{b}, R^{c}, R^{w}, n, and m are defined above and described in classes and subclasses herein.

In some embodiments, present disclosure includes a compound is of formula (Ib1), (IIb1), (Ib2), or (IIb2):

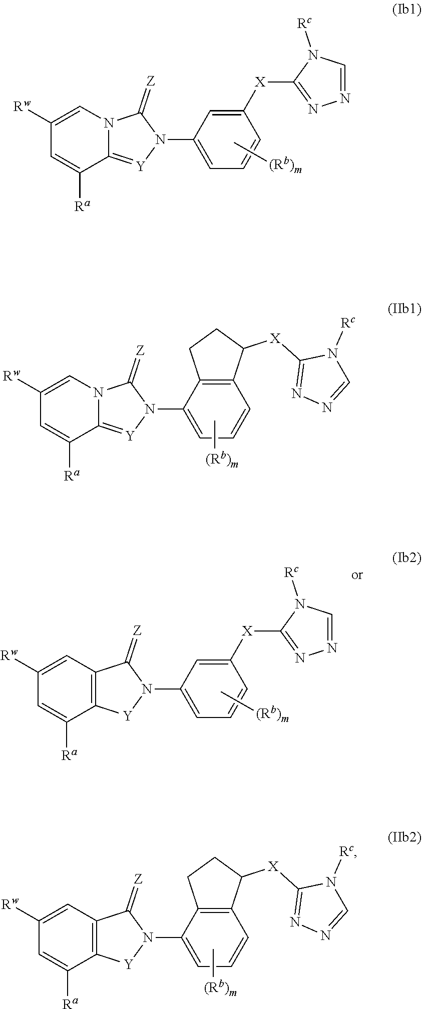

or pharmaceutically acceptable salts thereof, wherein X, Y, Z, R^{a}, R^{b}, R^{c}, R^{w}, and m are defined above and described in classes and subclasses herein.

In some embodiments, present disclosure includes a compound of formula (Ic) or (IIc):

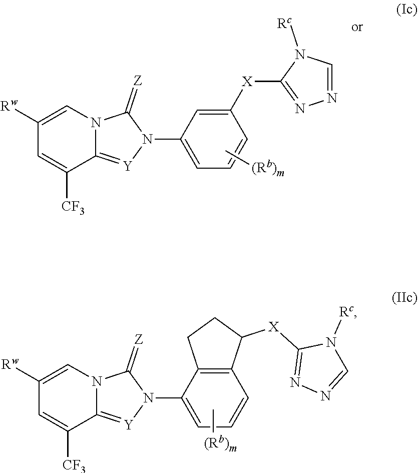

or a pharmaceutically acceptable salt thereof, wherein X, Y, Z, R^{a}, R^{b}, R^{c}, R^{w}, and m are defined above and described in classes and subclasses herein.

In some embodiments, present disclosure includes a compound of formula (Ic1) or (IIc1), (Ic2), or (IIc2):

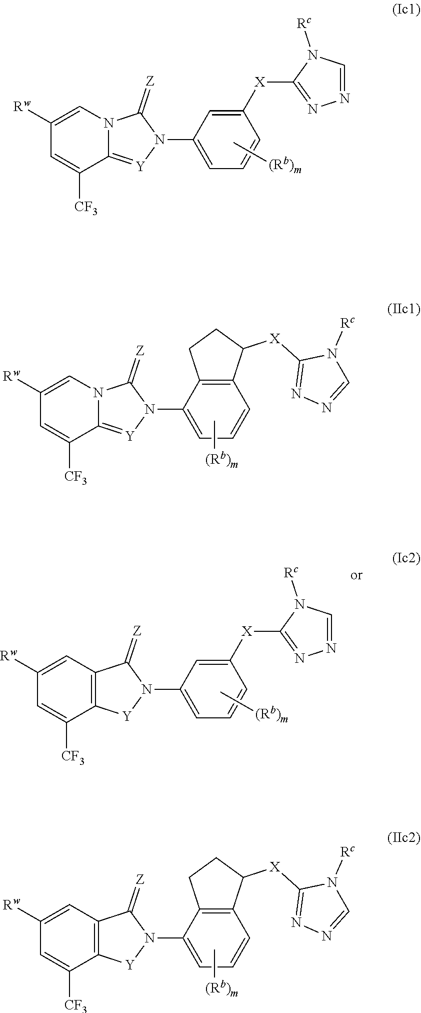

or a pharmaceutically acceptable salt thereof, wherein X, R^{a}, R^{b}, R^{c}, R^{w}, and m are defined above and described in classes and subclasses herein.

### X

In some embodiments, X is an optionally substituted C₁-C₃ alkylene chain, wherein one or more methylene units is optionally replaced by —N(H)—, —N(R¹)—, —O—, —S—, —SO—, —SO₂—, optionally substituted 3-6-membered carbocyclyl, and optionally substituted 3-6-membered heterocyclyl, wherein X is optionally substituted with an optionally substituted group selected from the group consisting of halogen, C₁-C₃ aliphatic, phenyl, 3-6-membered heteroaryl, 3-6-membered heterocyclyl, and —(CH₂)(3-6-membered carbocyclyl). In some embodiments, X is an optionally substituted C₁-C₃ alkylene chain, wherein one or more methylene units is optionally replaced by —N(H)—, —N(R¹)—, —O—, —S—, —SO—, —SO₂—, optionally substituted 3-6-membered carbocyclyl, and optionally substituted 3-6-membered heterocyclyl. In some embodiments, X is an optionally substituted C₁-C₃ alkylene chain, wherein one or more methylene units is optionally replaced by —N(H)—, —N(R¹)—, —O—, —S—, —SO—, —SO₂—,

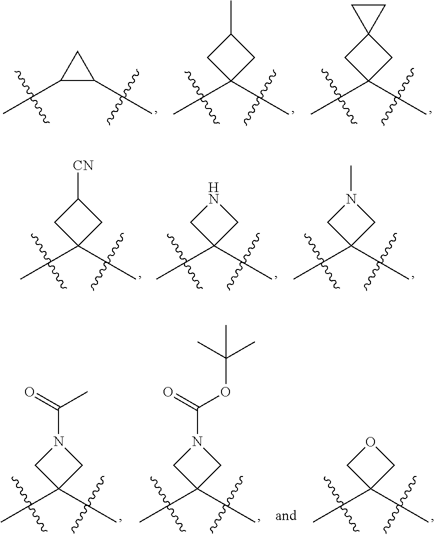

and wherein each methylene unit may be substituted with 1-2 substituents independently selected from the group consisting of halogen, optionally substituted C₁-C₃ aliphatic, optionally substituted 5-membered heteroaryl, optionally substituted phenyl, optionally substituted C₃-C₄ carbocylyl, and optionally substituted C₃-C₄ heterocyclyl. In some embodiments, In some embodiments, X is an optionally substituted C₁-C₃ alkylene chain, wherein one or more methylene units is optionally replaced by —N(H)—, —N(R¹)—, —O—, —S—,

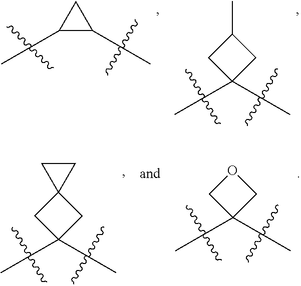

In some embodiments, X is an optionally substituted C₁-C₃ alkylene chain, wherein one or more methylene units is optionally replaced by —N(H)—, —N(R¹)—, —O—, —S—, —SO—, —SO₂—,

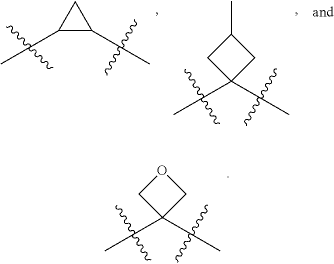

In some embodiments, X is optionally substituted C₁-C₂ alkylene. In some embodiments, X is

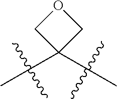 [SMILES: *C1(*)COC1]

or optionally substituted C₂ alkylene, wherein one methylene unit is replaced with

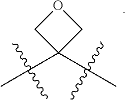 [SMILES: *C1(*)COC1]

In some embodiments, X is selected from the group consisting of

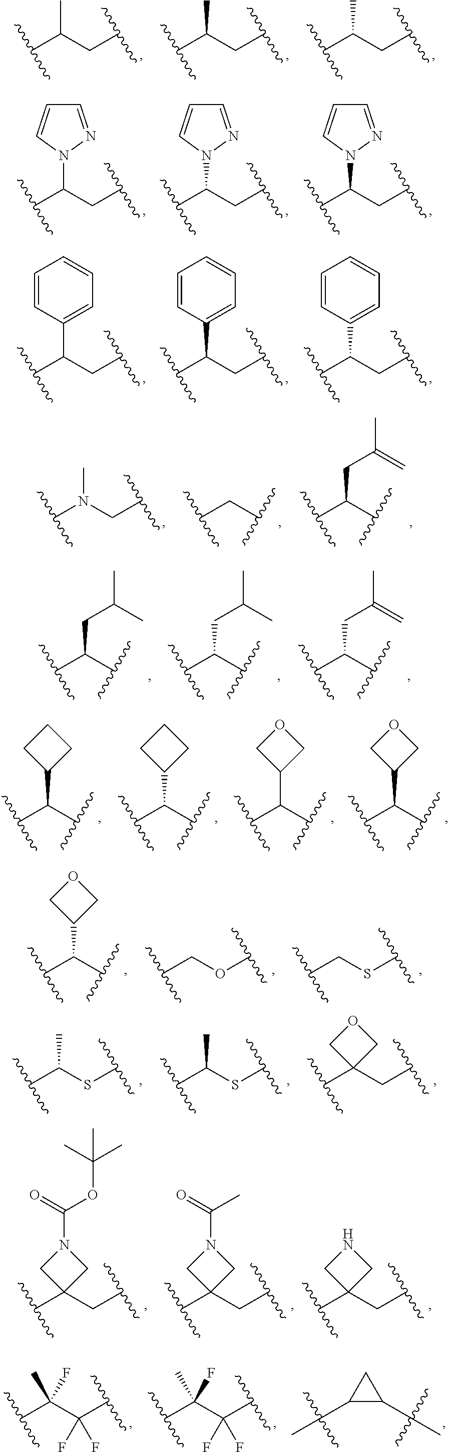

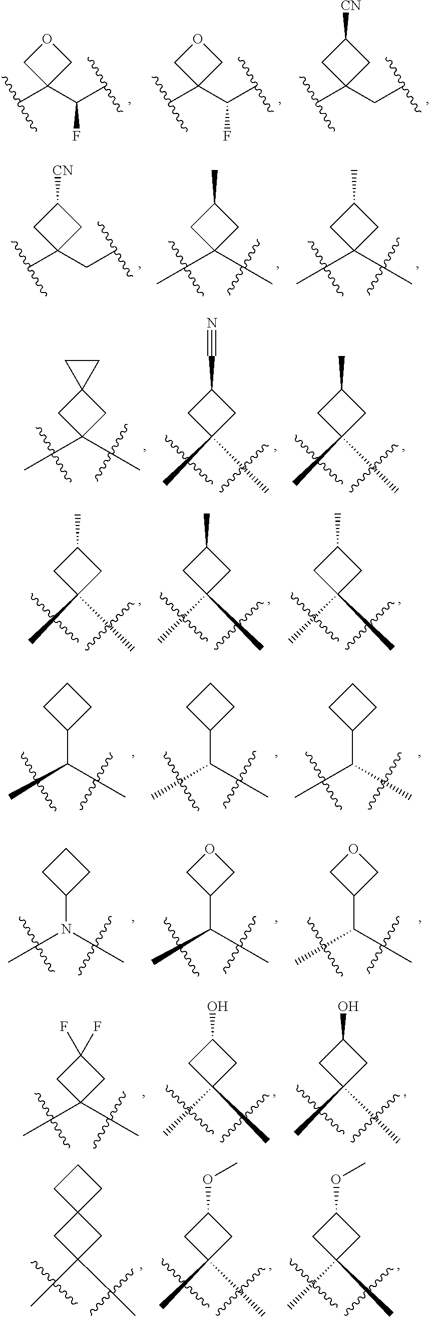

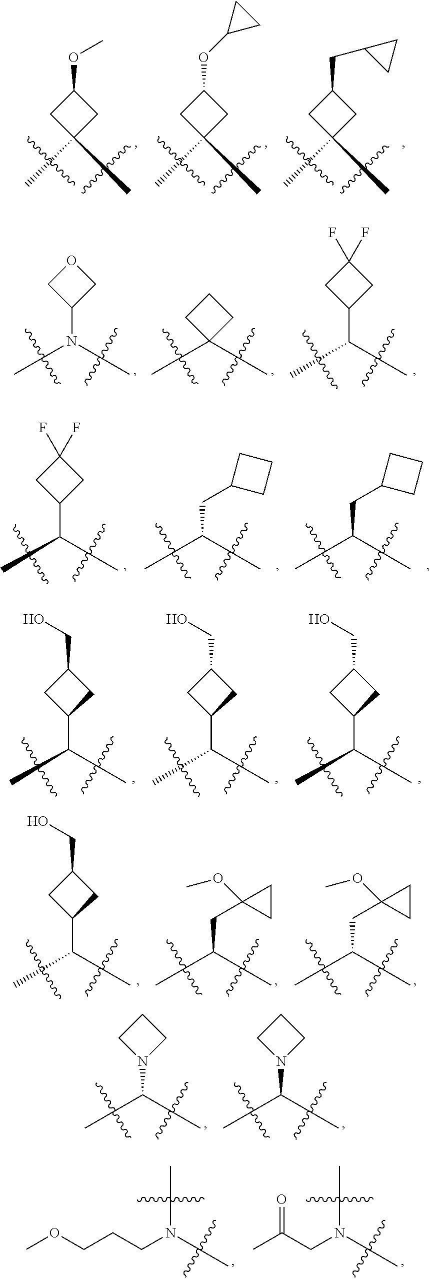

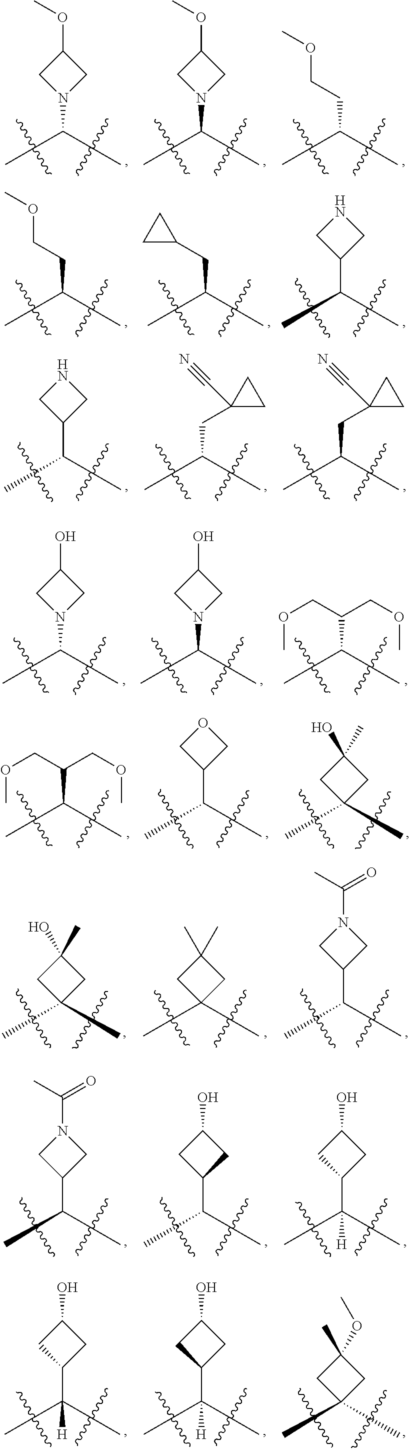

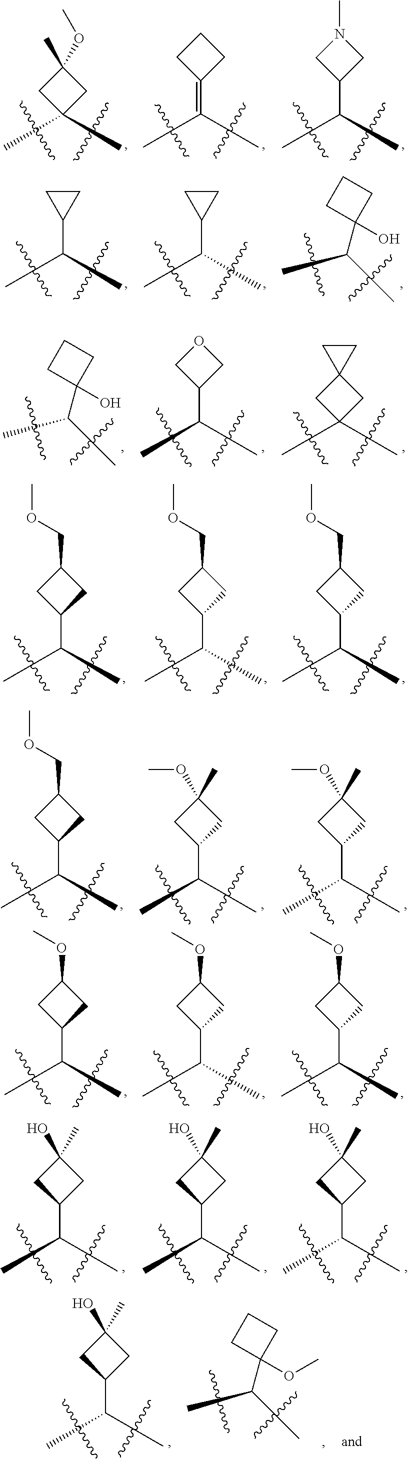

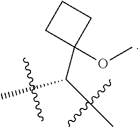 [SMILES: *[C@H](C)C1(OC)CCC1]

In some embodiments, wherein X is selected from the group consisting of

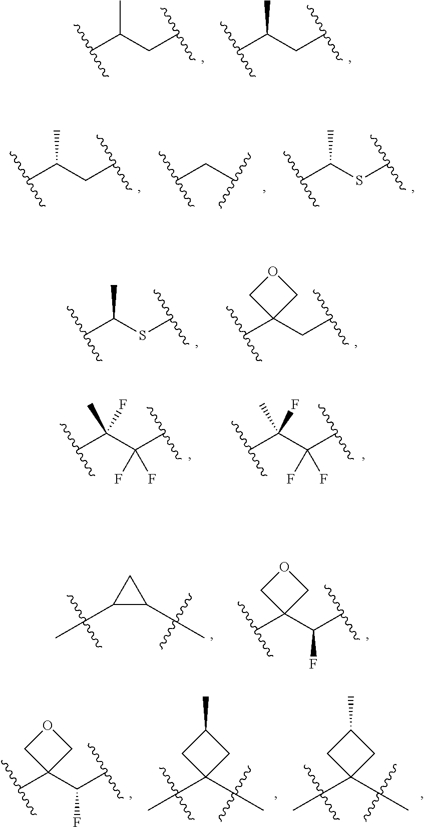

### L¹

In some embodiments, L¹ is a bond or an optionally substituted C₁-C₃ alkylene chain. In some embodiments, L¹ is a bond. In some embodiments, L is an optionally substituted C₁-C₃ alkylene chain. In some embodiments, L is —CH₂— or —CH(CH₃)—.

### L²

In some embodiments, L² is an optionally substituted C₁-C₁₂ alkylene chain, wherein 1-6 methylene units of L² are optionally and independently replaced by —C(O)—, —N(H)—, —N(R¹)—, —O—, —S—, —SO—, —SO₂—, optionally substituted 3-6-membered carbocyclyl, and optionally substituted 3-6-membered heterocyclyl, wherein X is optionally substituted with an optionally substituted group selected from the group consisting of halogen, C₁-C₃ aliphatic, phenyl, 3-6-membered heteroaryl, and 3-6-membered heterocyclyl. In some embodiments, L² is an optionally substituted C₁-C₁₂ alkylene chain, wherein 1-6 methylene units of L² are optionally and independently replaced by —N(H)—, —N(R¹)—, or —O—. In some embodiments, L² is an optionally substituted C₁-C₁₂ alkylene chain, wherein 1-6 methylene units of L² are optionally and independently replaced by —N(H)—, —N(R¹)—, —O—, or optionally substituted phenylene. In some embodiments, L² is an optionally substituted C₁-C₁₂ alkylene chain, wherein 1-6 methylene units of L² are optionally and independently replaced by —N(H)—, —N(R¹)—, —O—, or optionally substituted 3-8-membered heterocyclyl. In some embodiments, L² is an optionally substituted C₁-C₁₂ alkylene chain, wherein 1-6 methylene units of L² are optionally and independently replaced by —N(H)—, —N(R¹)—, —O—, or optionally substituted 6-membered heterocyclyl. In some embodiments, L² is an optionally substituted C₁-C₁₂ alkylene chain, wherein 1-6 methylene units of L² are optionally and independently replaced by —O—, or optionally substituted 3-6-membered heterocyclyl. In some embodiments, L² is an optionally substituted C₁-C₁₂ alkylene chain, wherein 1-6 methylene units of L² are optionally and independently replaced by —O—, or optionally substituted 6-membered heterocyclyl. In some embodiments, L² is an optionally substituted C₁-C₁₂ alkylene chain, wherein 1-6 methylene units of L² are optionally and independently replaced by —N(H)—, —N(R¹)—, or optionally substituted 3-8-membered heterocyclyl. In some embodiments, L² is an optionally substituted C₁-C₁₂ alkylene chain, wherein 1-6 methylene units of L² are optionally and independently replaced by —N(H)—, —N(R¹)—, or optionally substituted 6-membered heterocyclyl. In some embodiments, L² is an optionally substituted C₁-C₁₂ alkylene chain, wherein 1-6 methylene units of L² are optionally and independently replaced by —N(H)—, —N(R¹)—, —O—, optionally substituted 6-membered heterocyclyl, or optionally substituted phenylene.

In some embodiments, L² is an optionally substituted C₁-C₁₂ alkylene chain, wherein 1-6 methylene units of L² are optionally and independently replaced by —N(H)—, —N(R¹)—, —O—,

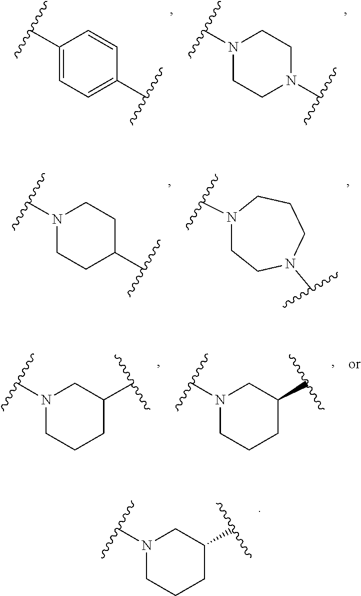

### A

In some embodiments, A is a bivalent group selected from the group consisting of a bond, optionally substituted C₃-C₇ carbocyclylene, optionally substituted C₁-C₆ heteroalkylene, optionally substituted 3-6 membered heterocyclylene containing 1-4 heteroatoms each selected from the group consisting of N, O, and S, optionally substituted phenylene, and optionally substituted 5-6-membered heteroarylene containing 1-4 heteroatoms each selected from the group consisting of N, O and S, wherein A is optionally substituted with 1-5 instances of R^{a1}. In some embodiments, A is selected from optionally substituted piperidine, optionally substituted tetrahydropyridine, optionally substituted pyrrolidine, optionally substituted dihydropyrrole, optionally substituted aziridine, and optionally substituted morpholine. In some embodiments, A is a bond.

### C

In some embodiments, C is optionally substituted 5-membered heteroaryl. In some embodiments, C is optionally substituted 5-membered heteroaryl containing 3 nitrogen atoms. In some embodiments, C is optionally substituted triazolyl. In some embodiments, C is optionally substituted 1,2,4 triazolyl. In some embodiments, C is optionally substituted 1,2,3 triazolyl. In some embodiments, C is optionally substituted 5-membered heteroaryl containing 2 nitrogen atoms. In some embodiments, C is optionally substituted pyrazolyl. In some embodiments, C is optionally substituted isoxazolyl. In some embodiments, C is optionally substituted thiazolyl. In some embodiments, C is optionally substituted thiadiazolyl. In some embodiments, C is optionally substituted 1,3,4 thiadiazolyl. In some embodiments, C is optionally substituted pyridinyl. In some embodiments, C is optionally substituted pyrazinyl. In some embodiments, C is optionally substituted pyrimidinyl. In some embodiments, C is optionally substituted pyridazinyl.

### G

In some embodiments, G is selected from the group consisting of optionally substituted C₃-C₁₀ carbocylyl, optionally substituted C₁-C₆ heteroalkyl, optionally substituted 3-10 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S, wherein G is optionally substituted with 1-5 instances of R^{g1}. In some embodiments, G is optionally substituted 9-membered heterocyclyl. In some embodiments, G is isoindoline or phthalimide. In some embodiments, G is selected from the group consisting of

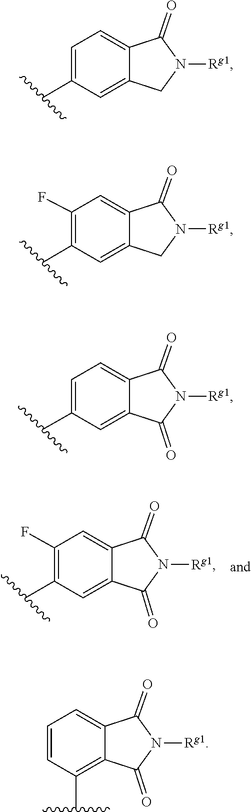

### R^{a}

In some embodiments, each R^{a} is independently selected from the group consisting of halogen, —CN, —OH, —OR¹, —NH₂, —NR¹R², —SH, —SR¹, —SF₅, —CO₂H, —CO₂R¹, —C(O)R¹, —CONH₂, —CONR¹R², —SO₂NH₂, —SO₂NR¹R², —SO₂OH, —SO₂OR¹, —S(O)R¹, —S(O)₂R¹, —S(O)(NH)R¹, —S(O)(NR¹)R¹, optionally substituted C₁-C₆ aliphatic, optionally substituted C₁-C₆ heteroalkyl, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S. In some embodiments, R^{a} is selected from halogen, —CN, —C(O)R¹, —CO₂H, —CONR¹R², optionally substituted C₁-C₆ aliphatic, and optionally substituted C₁-C₆ heteroalkyl. In some embodiments each R^{a} is independently selected from the group consisting of halogen, —CN, —CO₂H, —CHO, —CHF₂, —CF₃, —OMe, —S(O)₂NHMe,

In some embodiments, R^{a} is selected from the group consisting of halogen, —CN, and optionally substituted methyl.

### R^{b}

In some embodiments, each R^{b} is independently selected from the group consisting of, halogen, —CN, —OH, —OR¹, —NH₂, —NR¹R², —SH, —SR¹, —SF₅, —CO₂H, —CO₂R¹, —CONH₂, —CONR¹R², —SO₂NH₂, —SO₂NR¹R², —SO₂OH, —SO₂OR¹, —S(O)R¹, —S(O)₂R¹, —S(O)(NH)R¹, —S(O)(NR¹)R¹, optionally substituted C₁-C₆ aliphatic, optionally substituted C₁-C₆ heteroalkyl, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S.

### R^{c}

In some embodiments, each R^{c} is independently selected from the group consisting of hydrogen, optionally substituted C₁-C₆ aliphatic, —OR¹, —NH₂, —NR¹R², optionally substituted phenyl, optionally substituted 3-6 membered heterocyclyl containing 1-4 heteroatoms each selected from the group consisting of N, O, and S, optionally substituted 5-6-membered heteroaryl containing 1-4 heteroatoms each selected from the group consisting of N, O and S, —C(O)R³, —CO₂R³, —C(O)NHR³, and —SO₂R³. In some embodiments, each R^{c} is independently selected from the group consisting of hydrogen, optionally substituted C₁-C₆ aliphatic, optionally substituted phenyl, optionally substituted 3-6 membered heterocyclyl containing 1-4 heteroatoms each selected from the group consisting of N, O, and S, optionally substituted 5-6-membered heteroaryl containing 1-4 heteroatoms each selected from the group consisting of N, O and S, —C(O)R³, —CO₂R³, —C(O)NHR³, and —SO₂R³. In some embodiments, R^{c} is optionally substituted C₁-C₃ aliphatic. In some embodiments, R^{c} is methyl.

### R¹

In some embodiments, each R¹ is independently selected from the group consisting of optionally substituted C₁-C₆ aliphatic, optionally substituted phenyl, optionally substituted 3-6 membered heterocyclyl containing 1-4 heteroatoms each selected from the group consisting of N, O, and S, optionally substituted 5-6-membered heteroaryl containing 1-4 heteroatoms each selected from the group consisting of N, O and S, —C(O)R³, —CO₂R³, —C(O)NHR³, and —SO₂R³. In some embodiments, each R¹ is optionally substituted C₁-C₆ aliphatic. In some embodiments, each R¹ is methyl.

### R²

In some embodiments, each R² is independently selected from the group consisting of hydrogen, optionally substituted C₁-C₆ aliphatic, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S;
or R¹ and R² are taken together with their intervening atom(s) to form a 3-8-membered heterocyclyl ring containing 1-3 heteroatoms selected from the group consisting of N, O, and S, or an optionally substituted 5-6-membered heteroaryl ring containing 1-4 heteroatoms selected from the group consisting of N, O, and S.

In some embodiments, each R² is optionally substituted C₁-C₆ aliphatic. In some embodiments, each R² is methyl.

### R³

In some embodiments, each R³ is independently selected from the group consisting of optionally substituted C₁-C₆ aliphatic, optionally substituted 3-6 membered heterocyclyl containing 1-4 heteroatoms each selected from the group consisting of N, O, and S, optionally substituted phenyl, optionally substituted 5-6-membered heteroaryl containing 1-4 heteroatoms each selected from the group consisting of N, O and S.

In some embodiments, the present disclosure includes compounds described in Table 1.

TABLE 1

| Cmpd No | Structures |
|---|---|
| 1 | 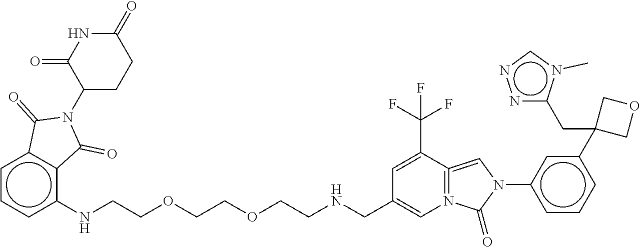 |
| 2 | 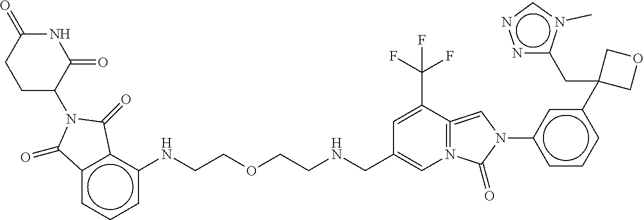 |
| 3 | 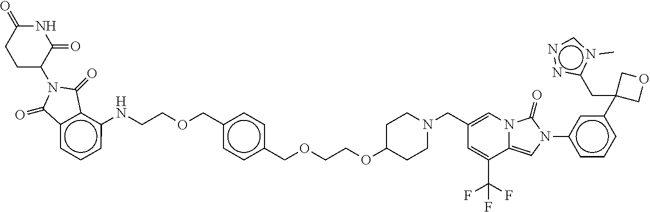 |
| 4 | 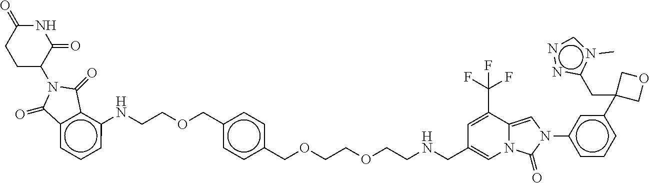 |
| 5 | 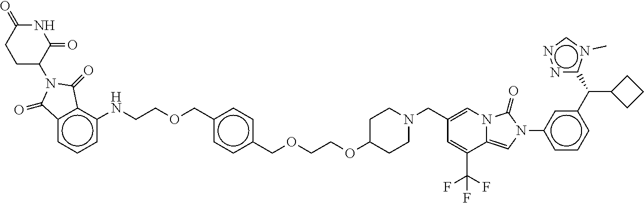 |
| 6 | 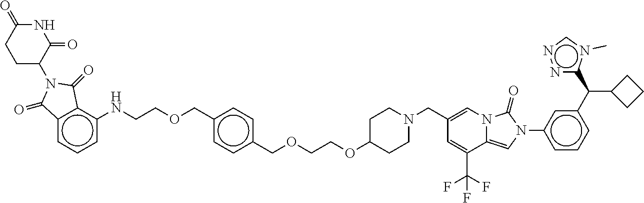 |
| 7 | 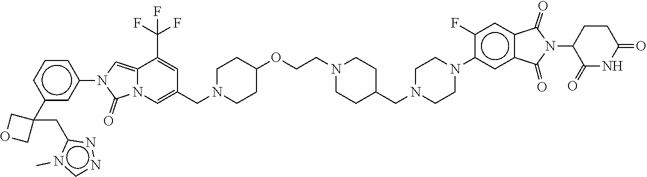 |
| 8 | 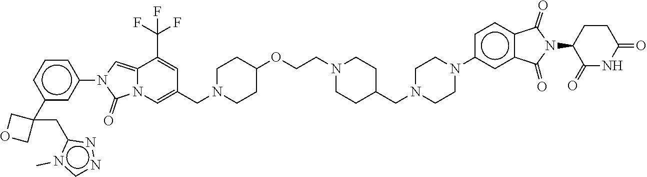 |
| 9 | 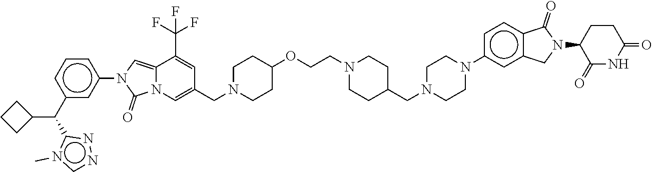 |
| 10 | 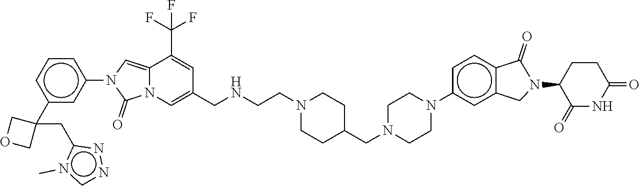 |
| 11 | 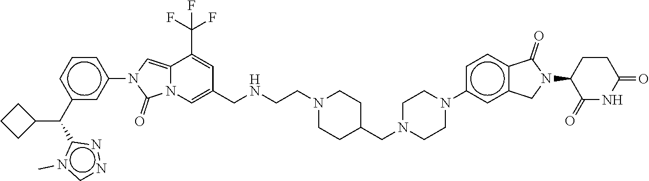 |
| 12 | 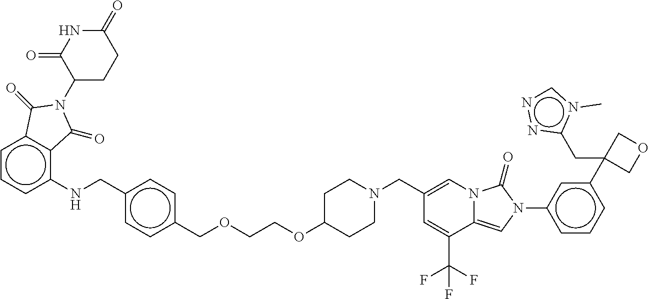 |
| 13 | 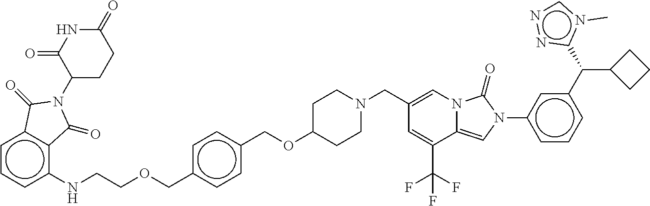 |
| 14 | 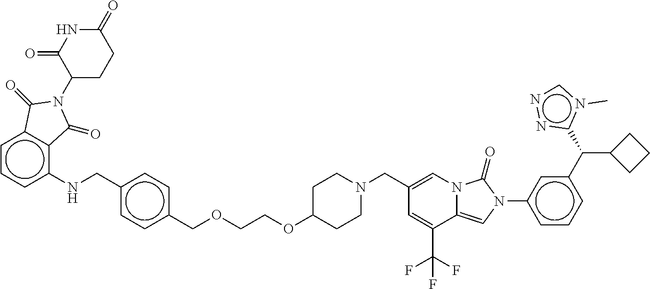 |
| 15 | 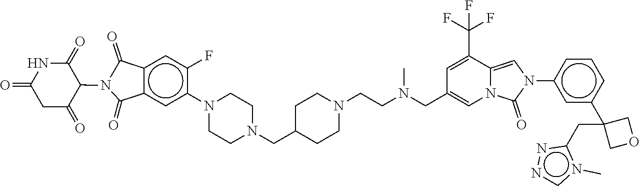 |
| 16 | 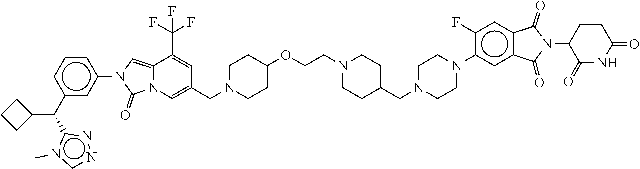 |
| 17 | 57 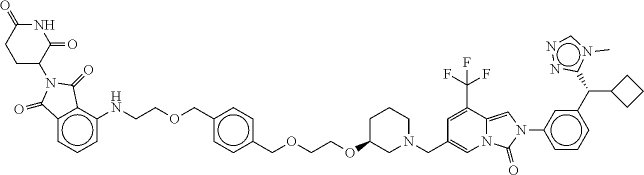 |
| 18 | 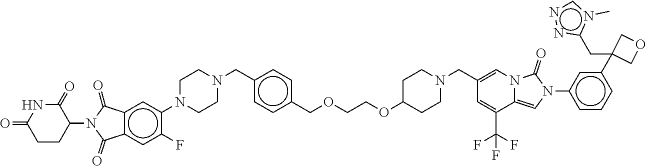 |
| 19 | 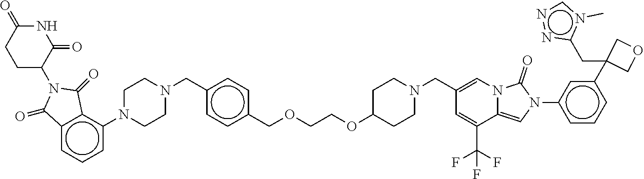 |
| 20 | 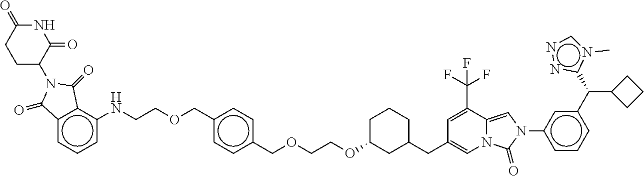 |
| 21 | 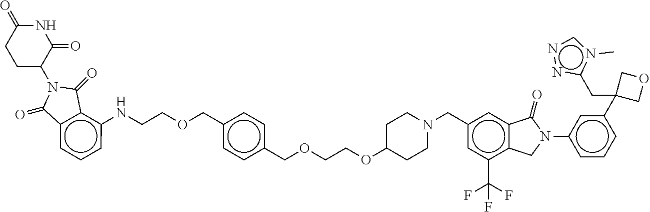 |
| 22 | 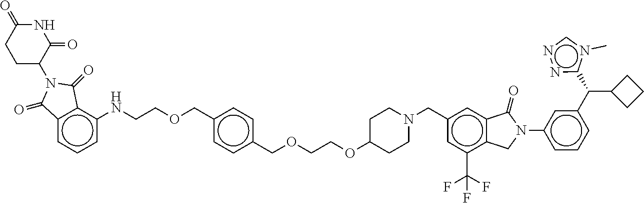 |
| 23 | 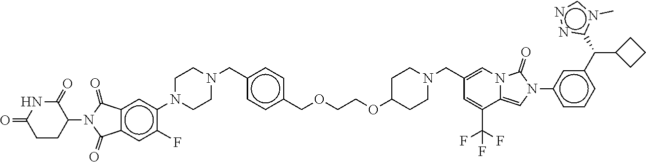 |
| 24 | 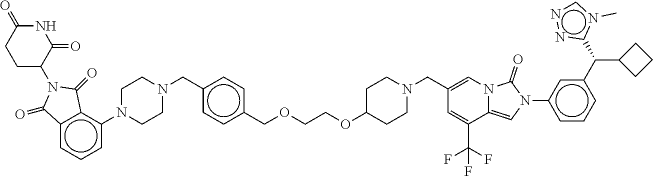 |
| 25 | 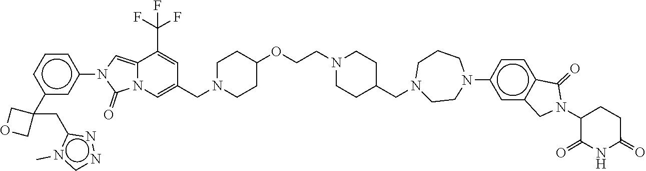 |
| 26 | 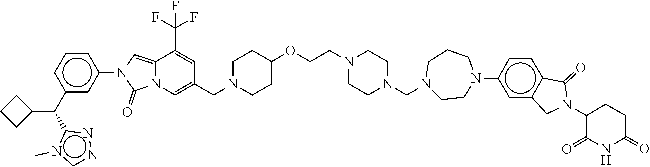 |
| 27 | 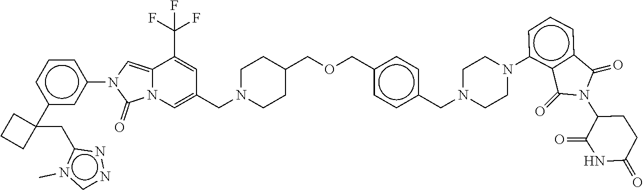 |
| 28 | 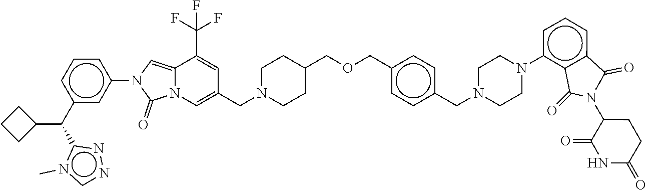 |
| 29 | 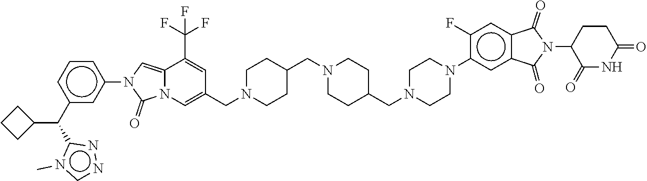 |
| 30 | 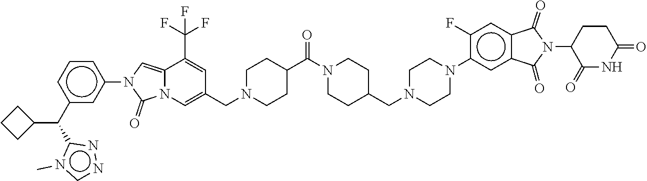 |
| 31 | 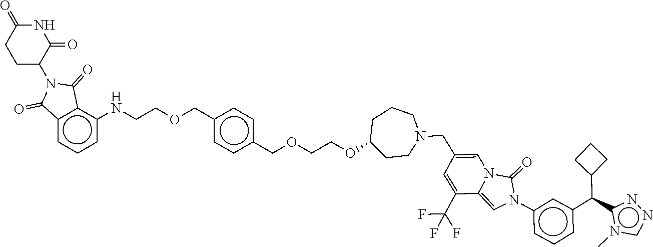 |
| 32 | 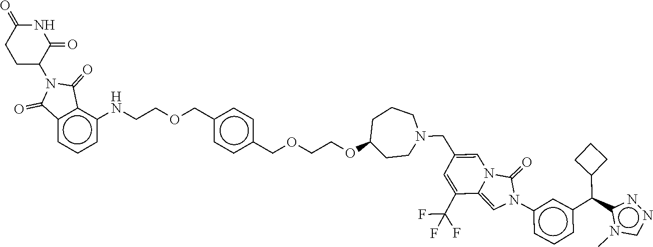 |
| 33 | 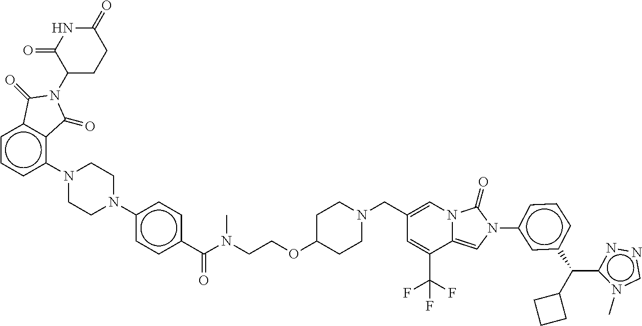 |
| 34 | 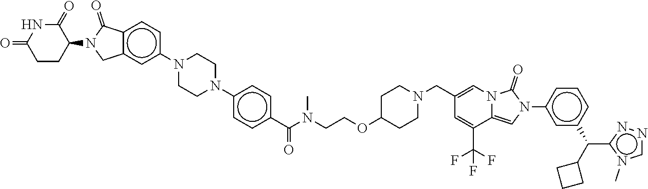 |
| 35 | 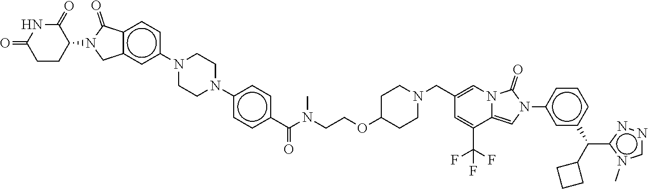 |
| 36 | 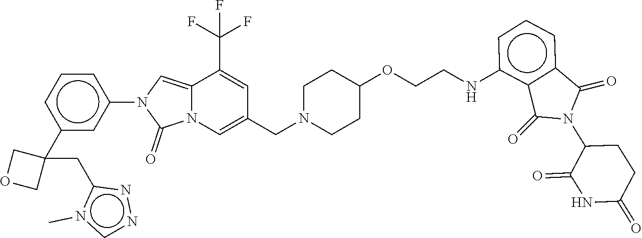 |
| 37 | 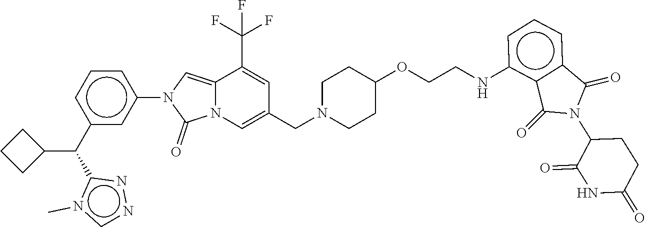 |
| 38 | 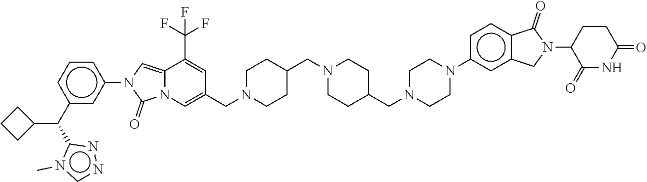 |
| 39 | 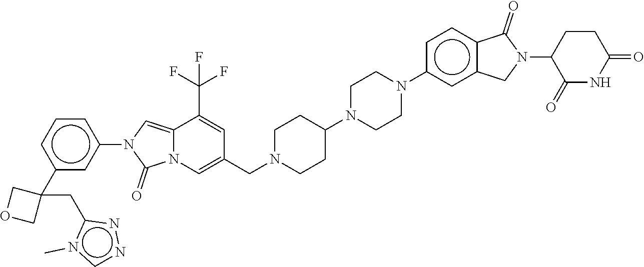 |
| 40 | 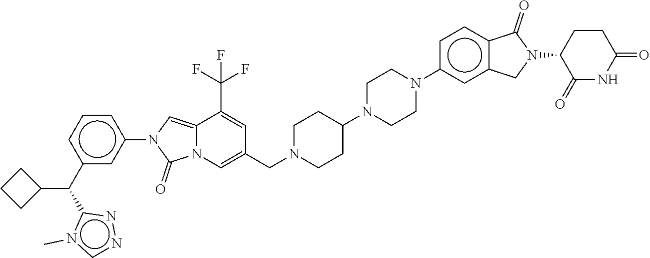 |
| 41 | 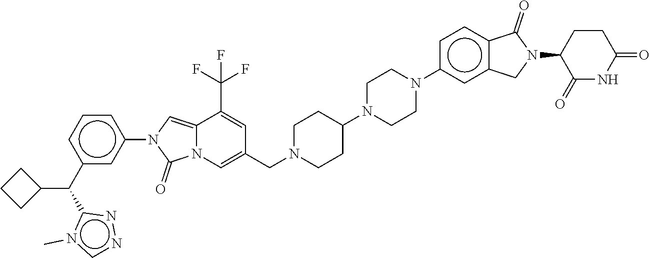 |
| 42 | 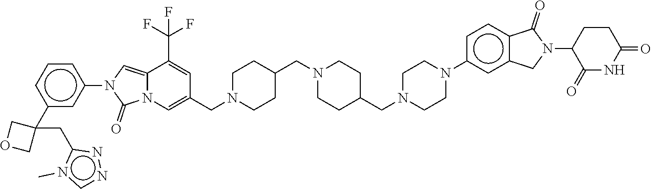 |
| 43 | 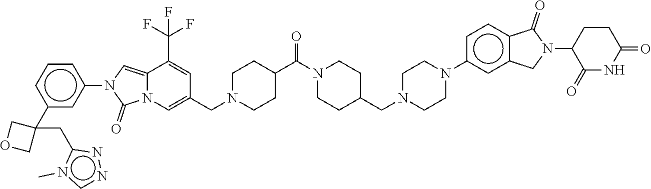 |
| 44 | 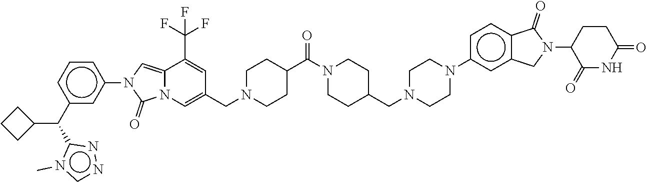 |
| 45 | 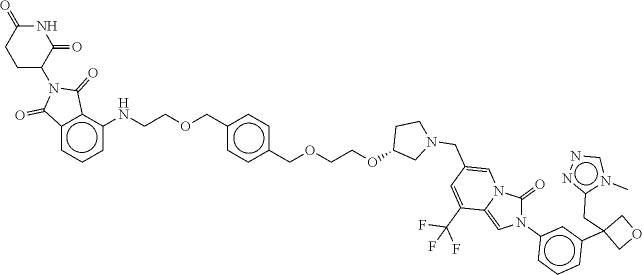 |
| 46 | 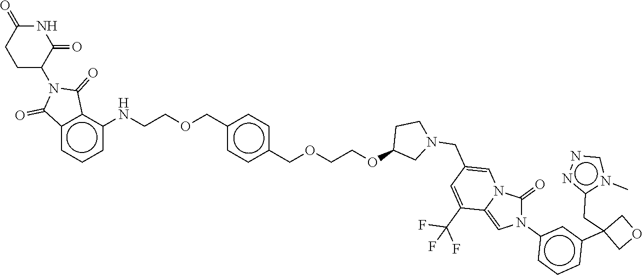 |
| 47 | 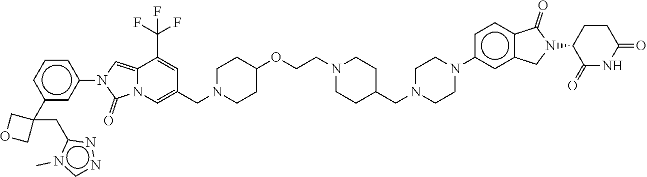 |
| 48 | 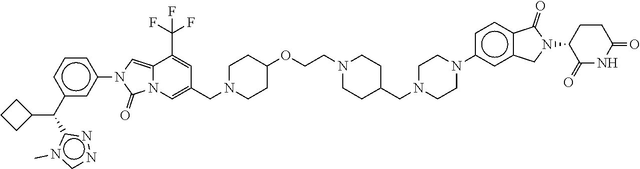 |
| 49 | 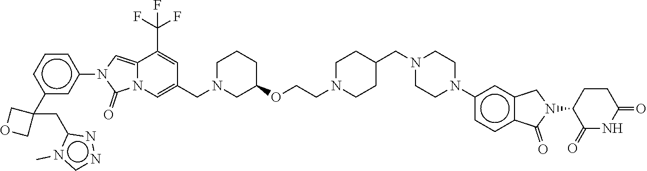 |
| 50 | 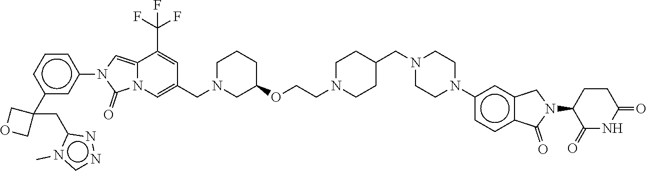 |
| 51 | 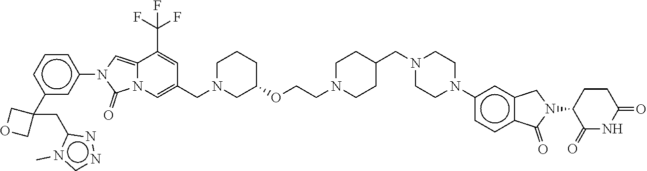 |
| 52 | 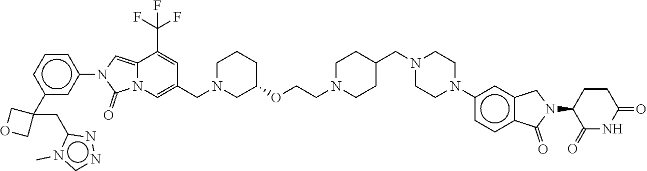 |
| 53 | 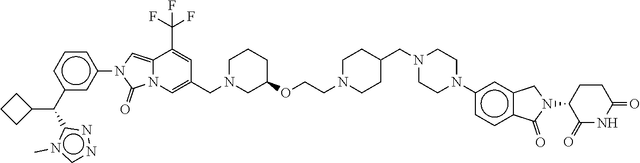 |
| 54 | 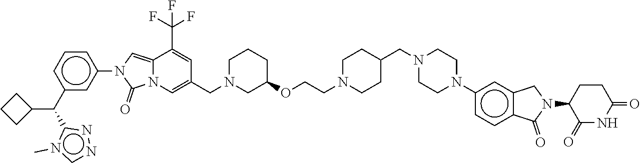 |
| 55 | 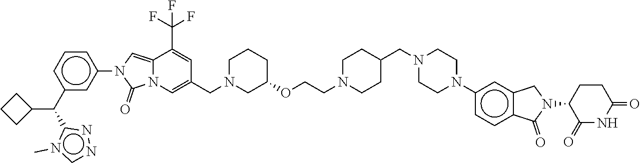 |
| 56 | 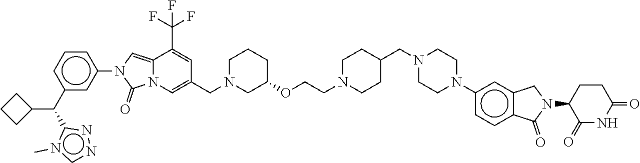 |
| 57 | 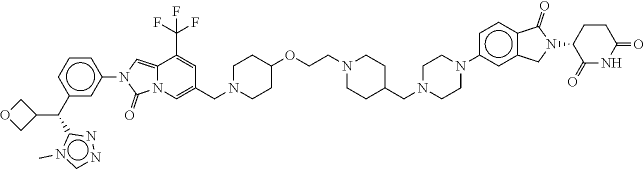 |
| 58 | 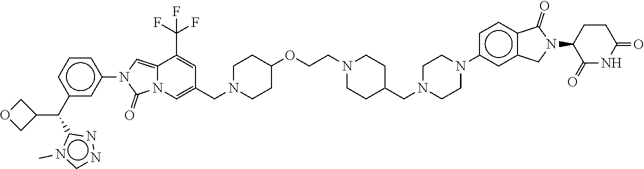 |
| 59 | 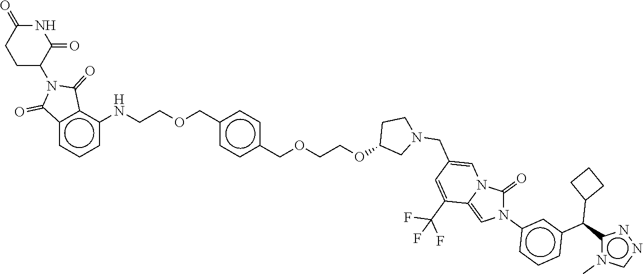 |
| 60 | 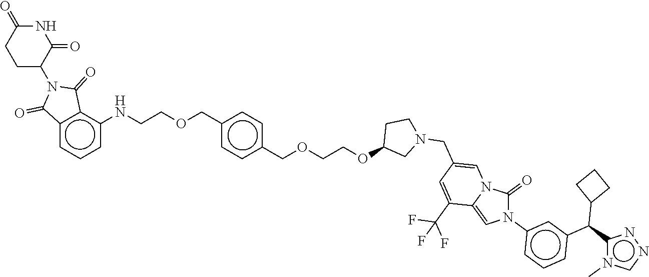 |
| 61 | 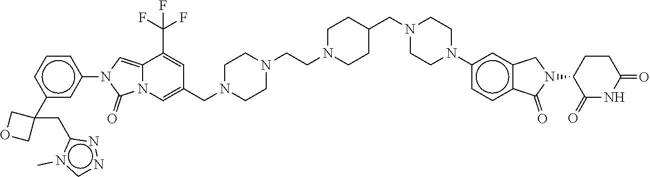 |
| 62 | 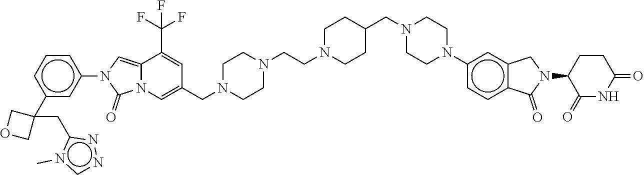 |
| 63 | 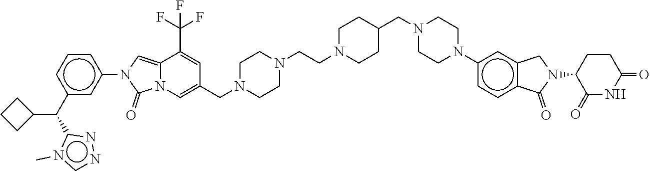 |
| 64 | 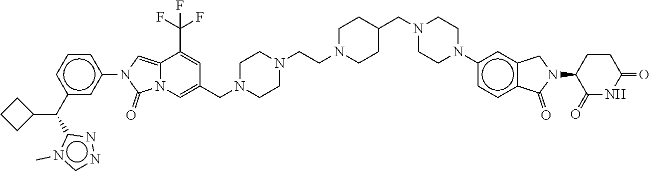 |
| 65 | 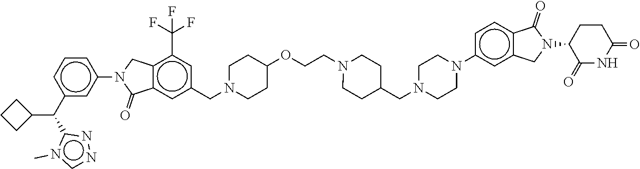 |
| 66 | 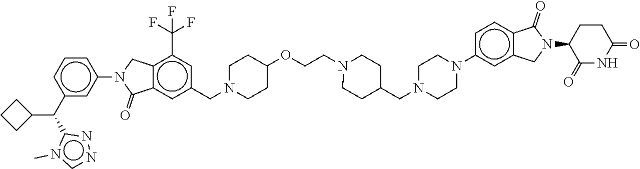 |
| 67 | 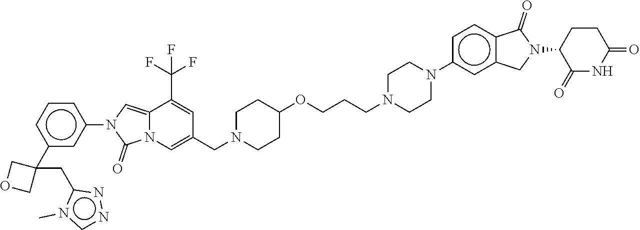 |
| 68 | 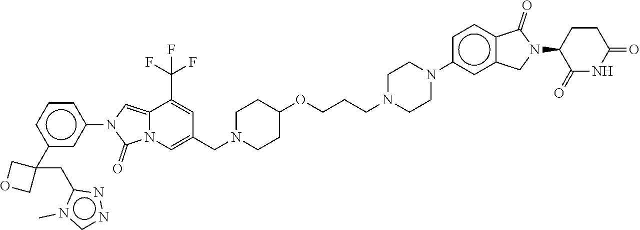 |
| 69 | 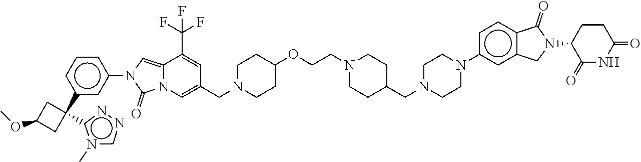 |
| 70 | 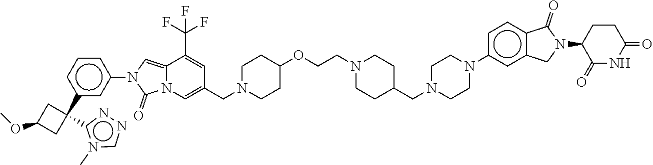 |
| 71 | 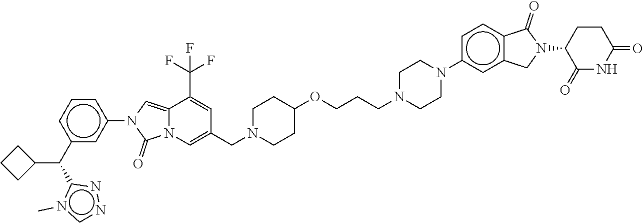 |
| 72 | 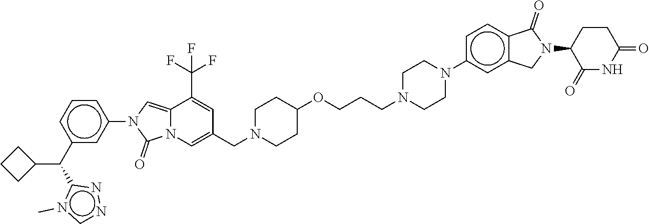 |
| 73 | 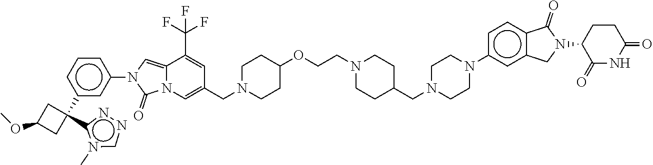 |
| 74 | 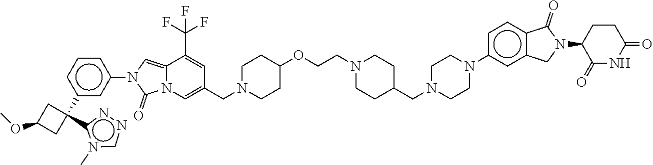 |
| 75 | 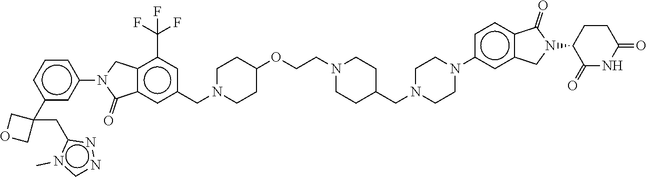 |
| 76 | 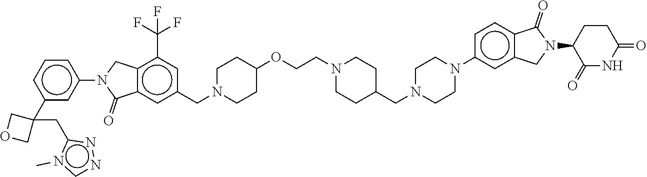 |
| 77 | 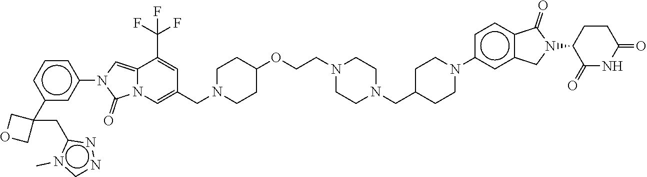 |
| 78 | 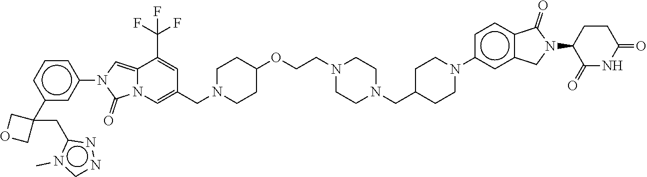 |
| 79 | 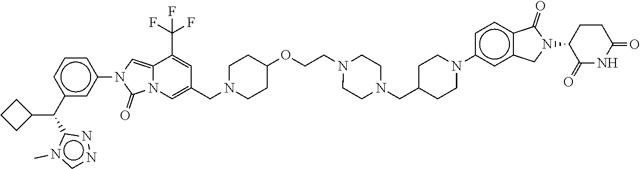 |
| 80 | 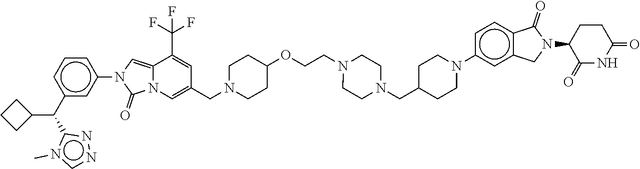 |
| 81 | 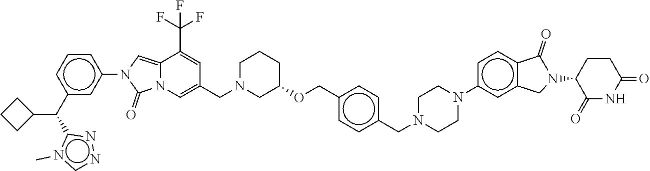 |
| 82 | 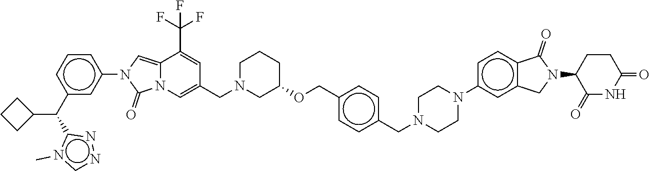 |
| 83 | 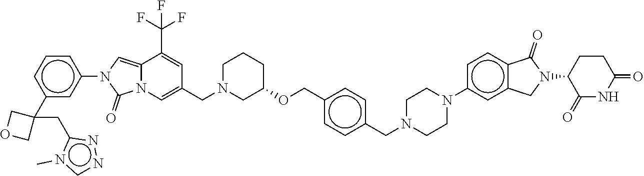 |
| 84 | 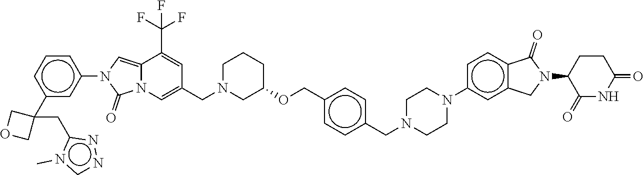 |
| 85 | 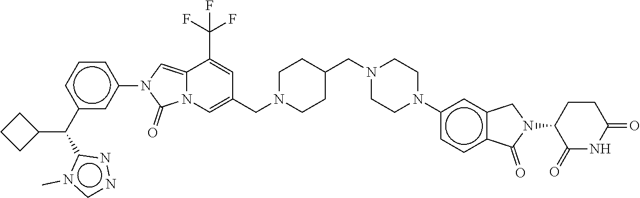 |
| 86 | 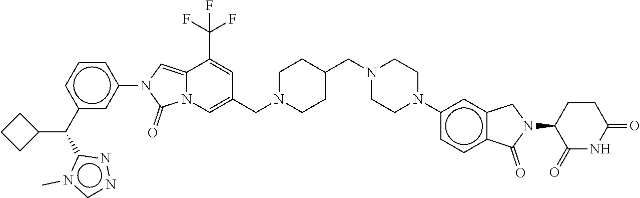 |
| 87 | 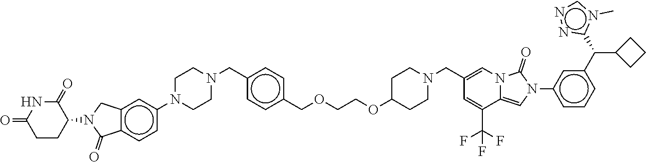 |
| 88 | 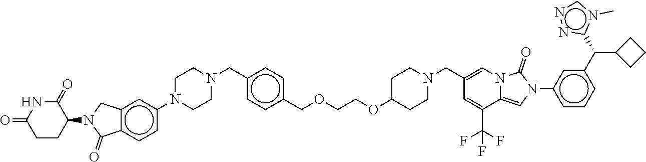 |
| 89 | 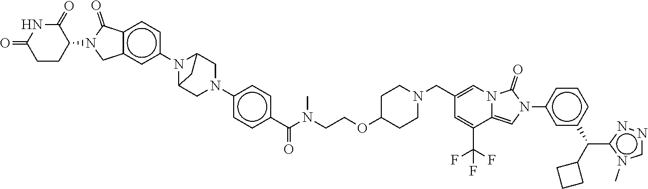 |
| 90 | 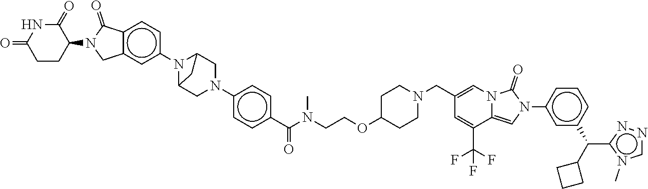 |
| 91 | 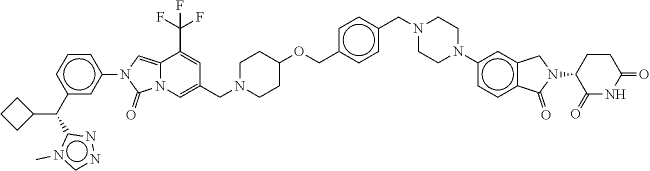 |
| 92 | 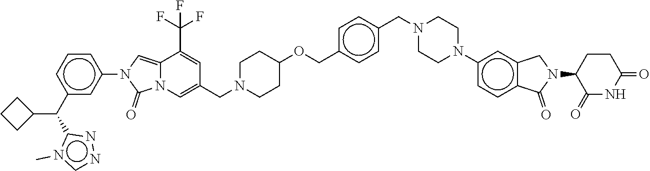 |
| 93 | 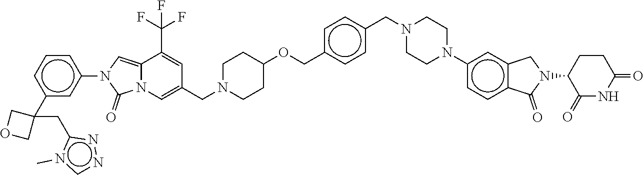 |
| 94 | 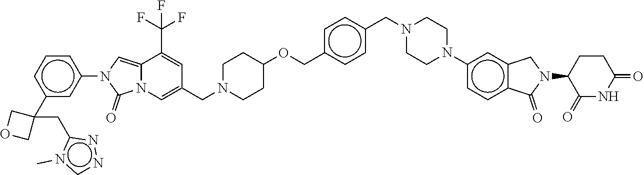 |
| 95 | 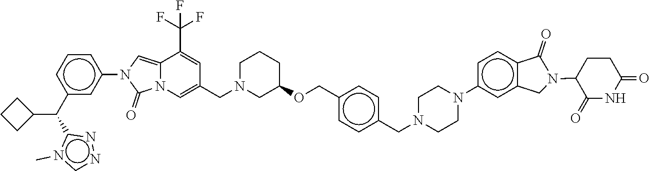 |
| 96 | 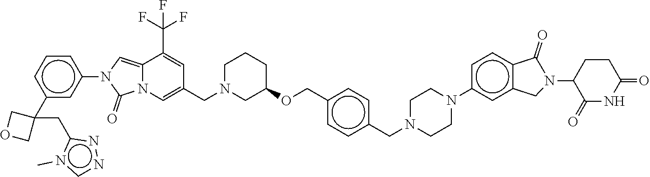 |
| 97 | 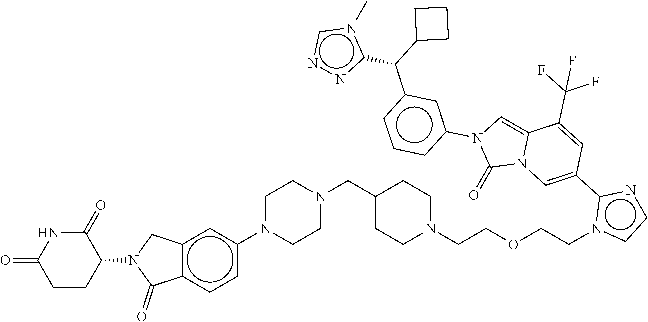 |
| 98 | 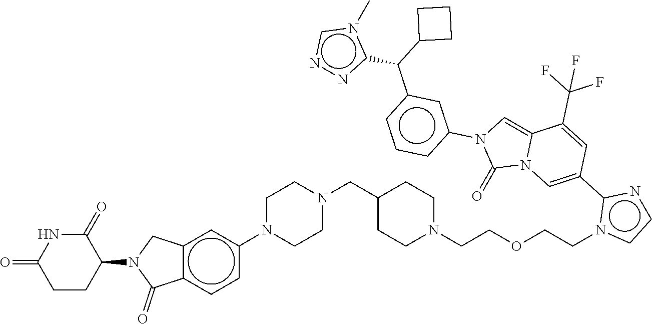 |
| 99 | 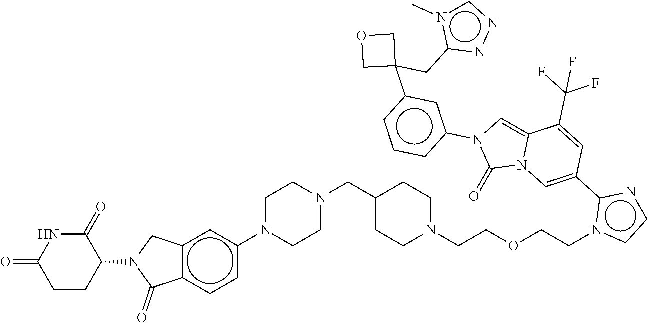 |
| 100 | 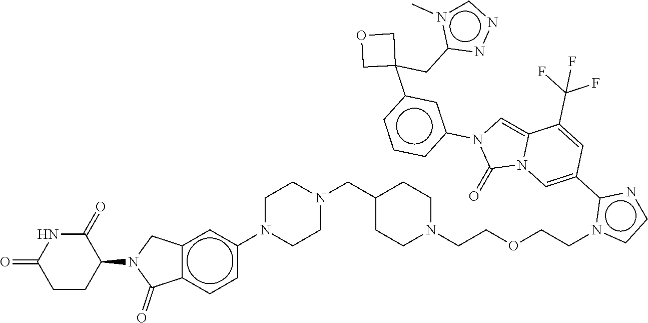 |
| 101 | 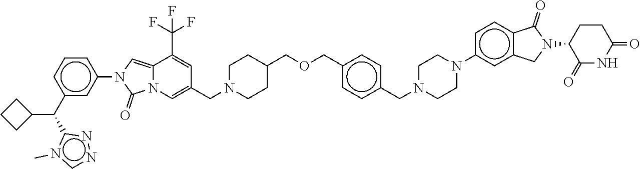 |
| 102 | 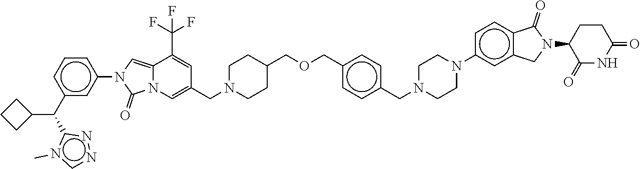 |
| 103 | 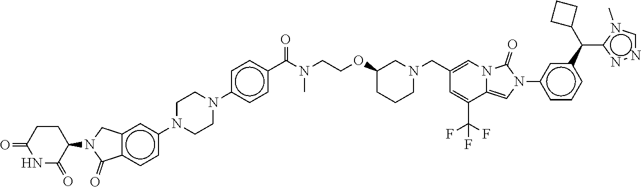 |
| 104 | 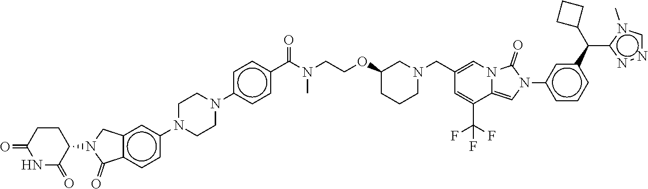 |
| 105 | 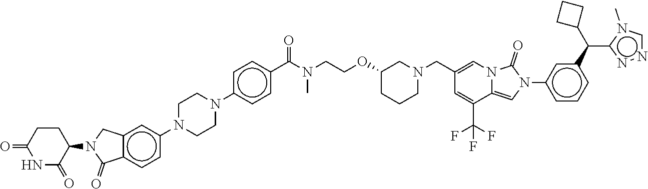 |
| 106 | 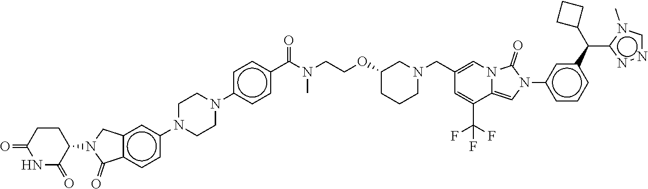 |
| 107 | 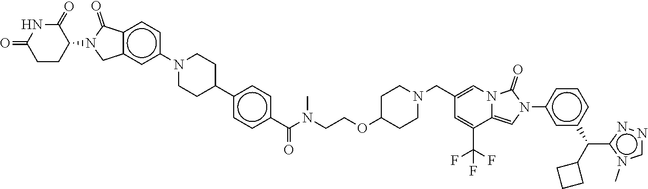 |
| 108 | 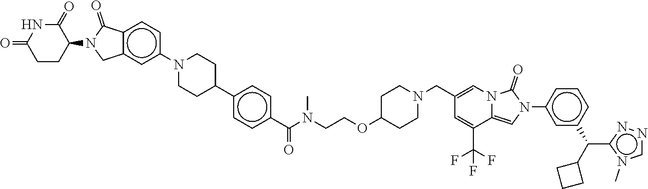 |
| 109 | 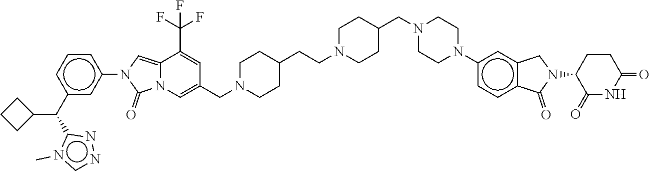 |
| 110 | 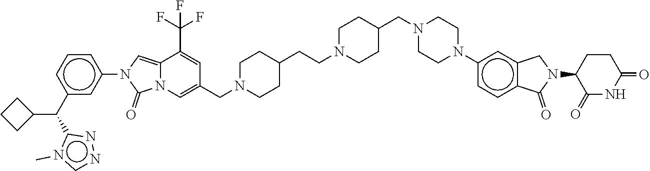 |
| 111 | 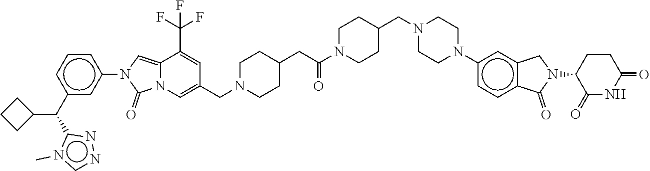 |
| 112 | 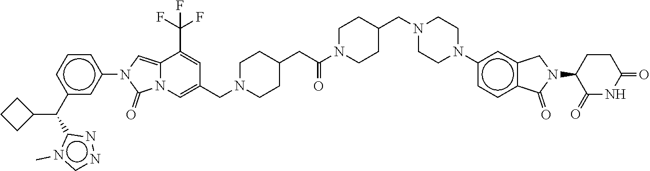 |
| 113 | 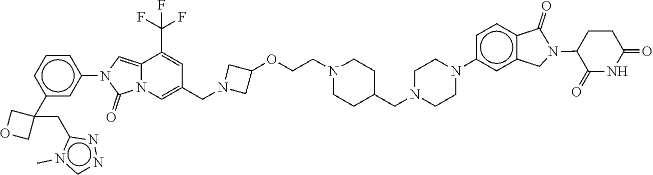 |
| 114 | 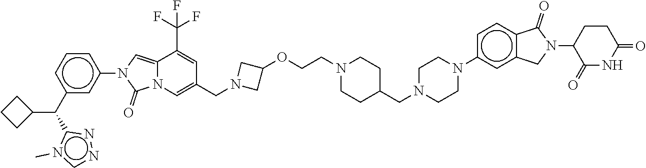 |
| 115 | 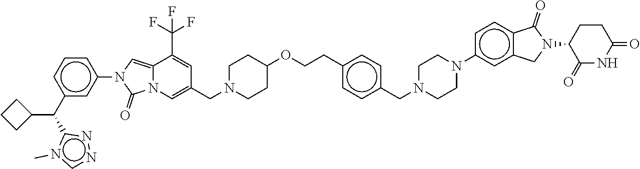 |
| 116 | 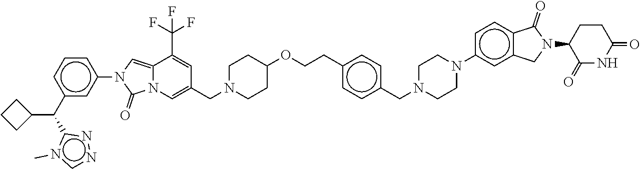 |
| 117 | 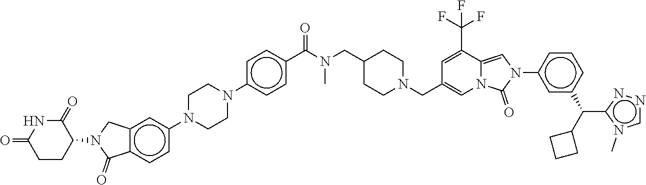 |
| 118 | 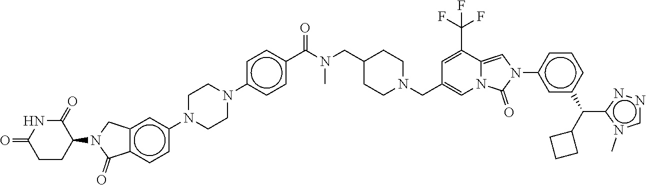 |
| 119 | 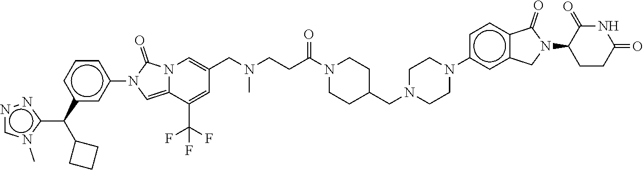 |
| 120 | 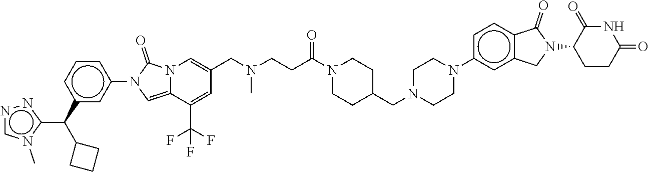 |
| 121 | 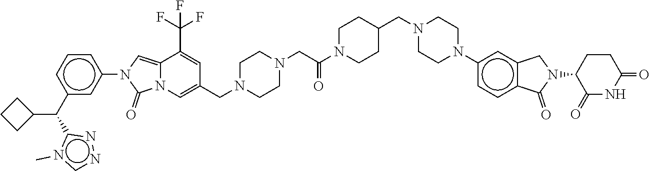 |
| 122 | 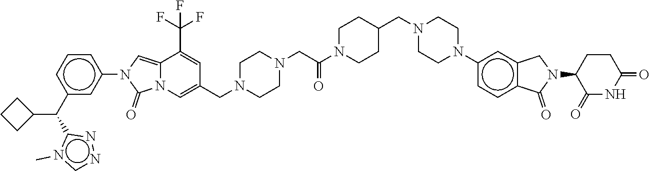 |
| 123 | 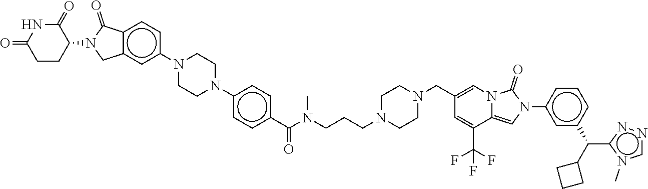 |
| 124 | 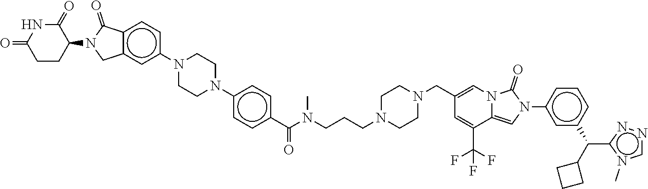 |
| 125 | 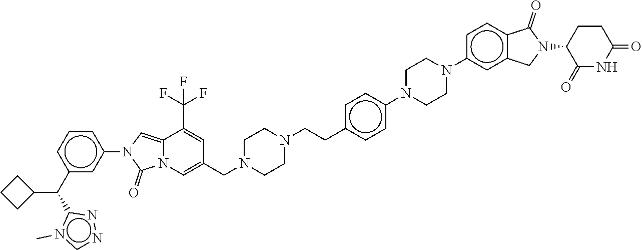 |
| 126 | 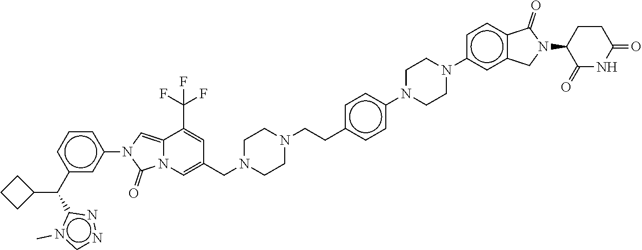 |
| 127 | 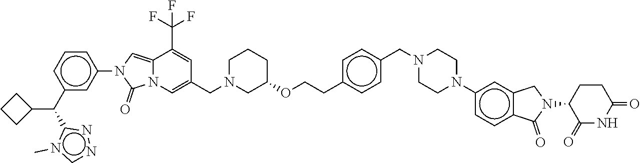 |
| 128 | 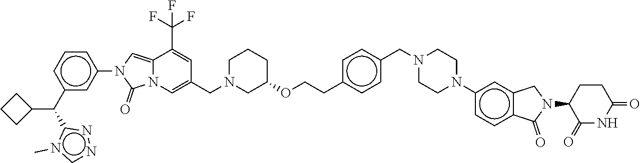 |
| 129 | 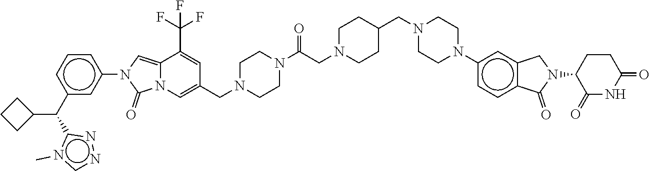 |
| 130 | 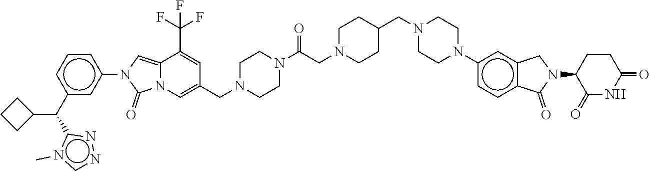 |
| 131 | 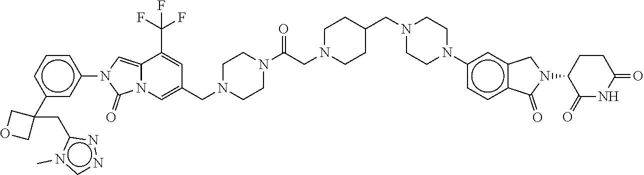 |
| 132 | 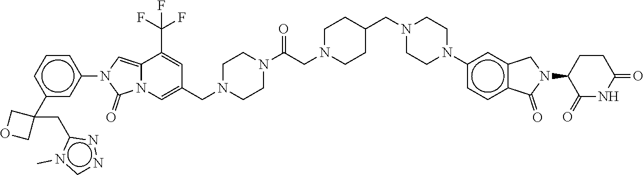 |
| 133 | 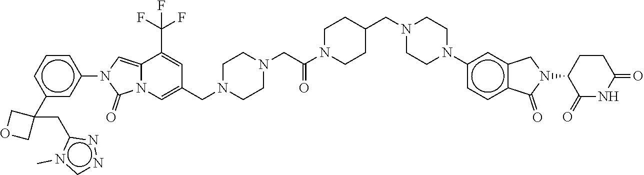 |
| 134 | 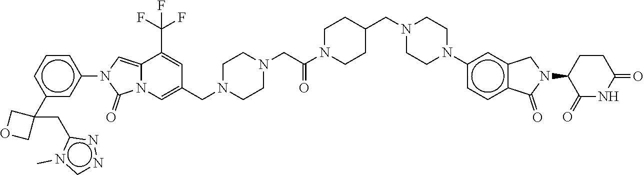 |
| 135 | 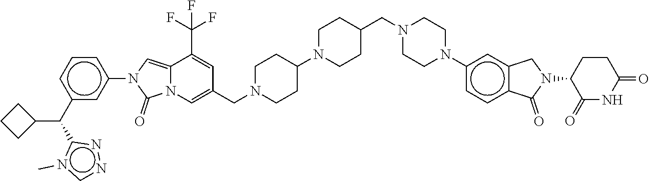 |
| 136 | 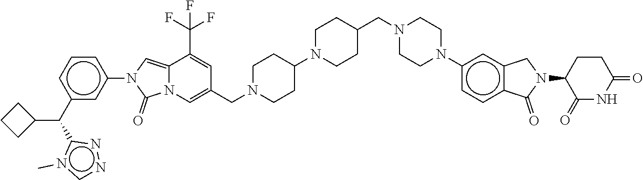 |
| 137 | 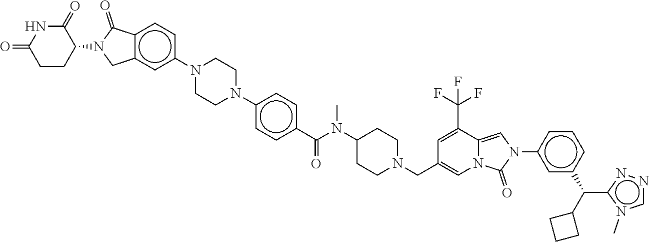 |
| 138 | 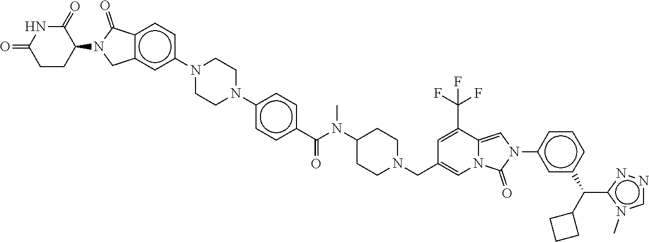 |
| 139 | 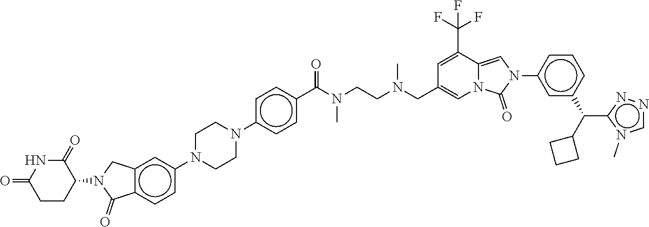 |
| 140 | 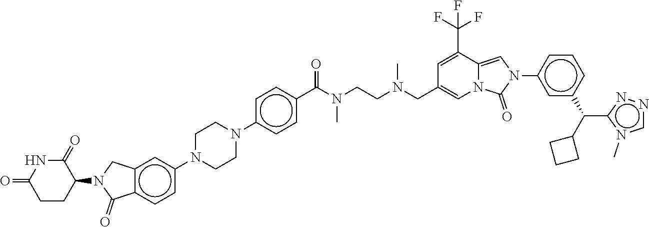 |
| 141 | 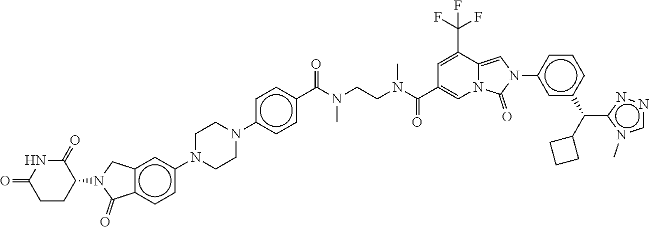 |
| 142 | 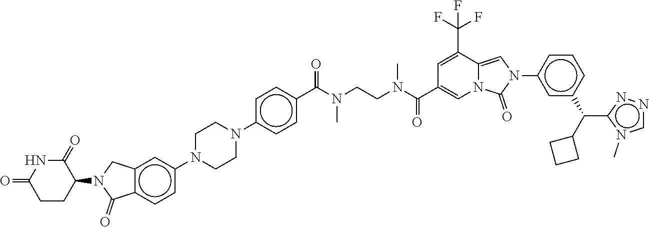 |
| 143 | 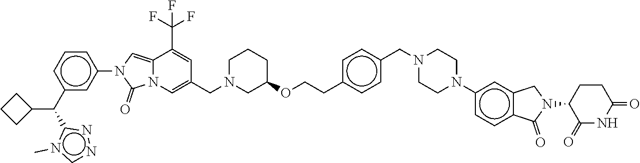 |
| 144 | 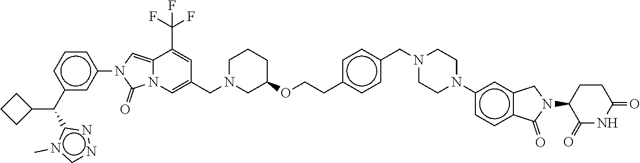 |
| 145 | 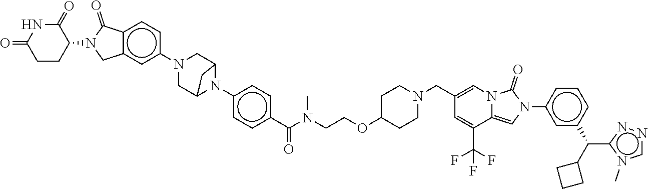 |
| 146 | 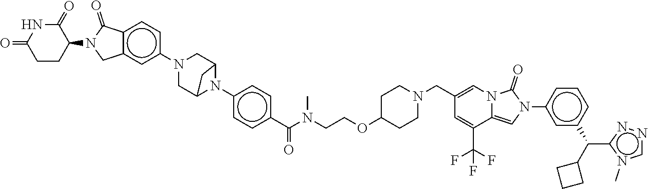 |
| 147 | 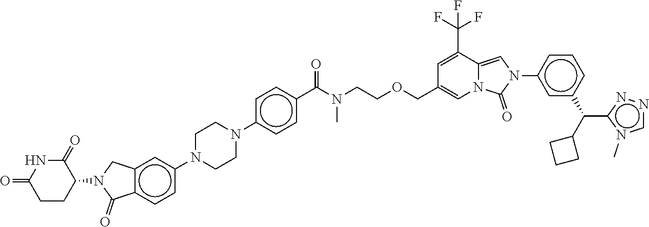 |
| 148 | 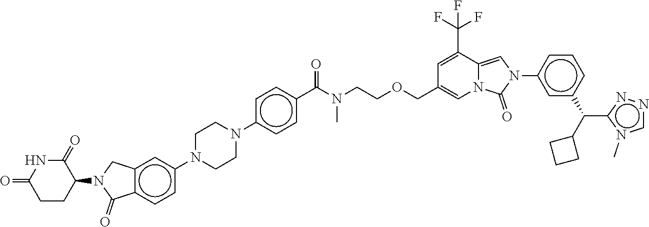 |
| 149 | 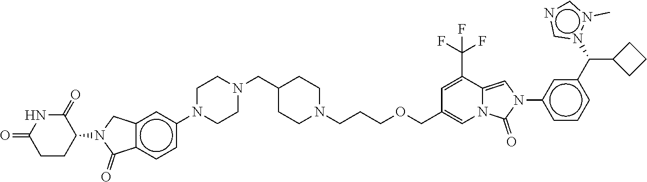 |
| 150 | 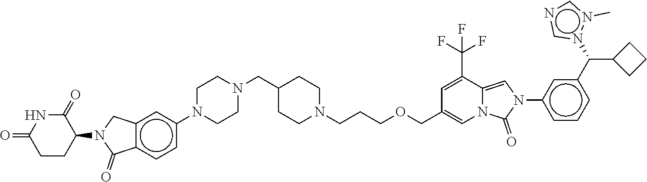 |
| 151 | 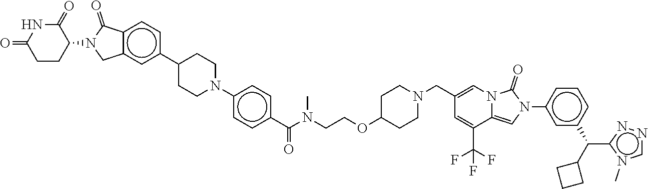 |
| 152 | 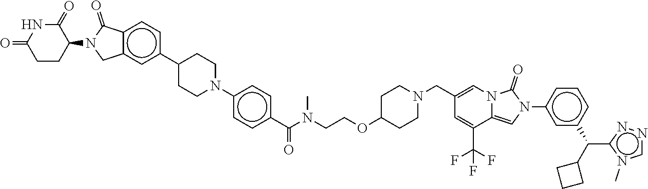 |
| 153 | 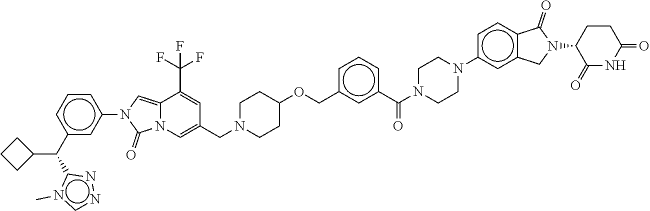 |
| 154 | 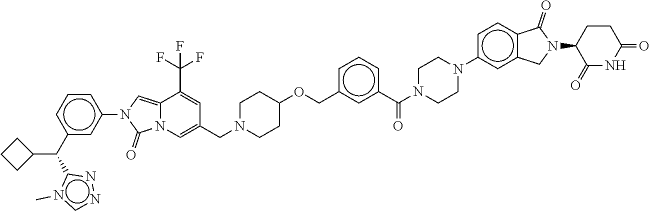 |
| 155 | 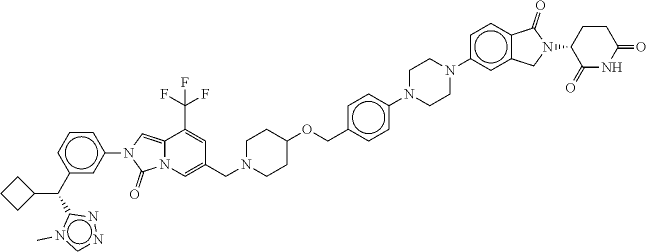 |
| 156 | 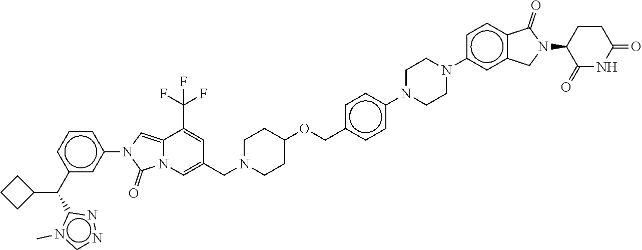 |
| 157 | 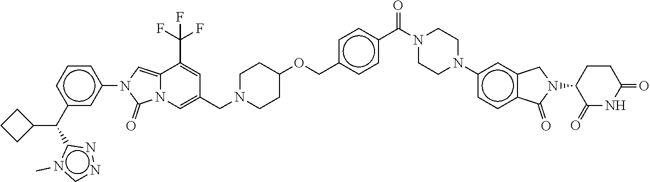 |
| 158 | 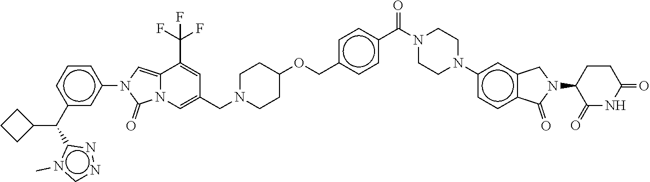 |
| 159 | 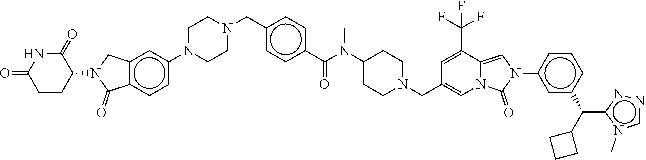 |
| 160 | 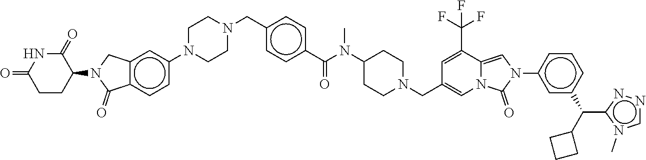 |
| 161 | 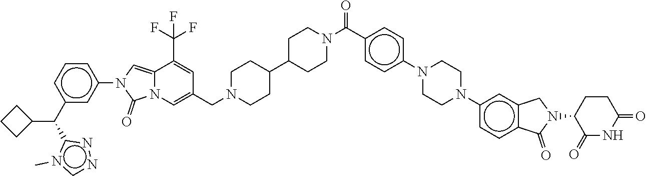 |
| 162 | 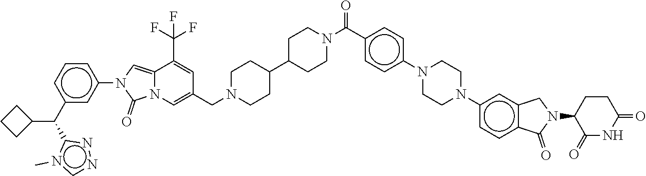 |
| 163 | 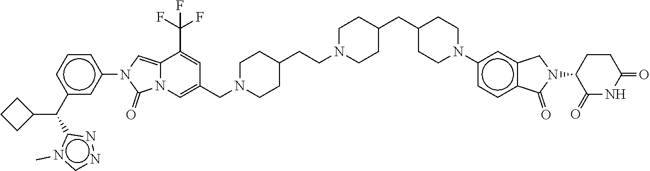 |
| 164 | 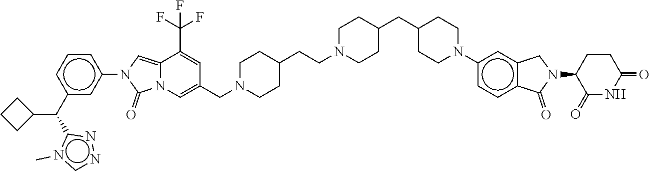 |
| 165 | 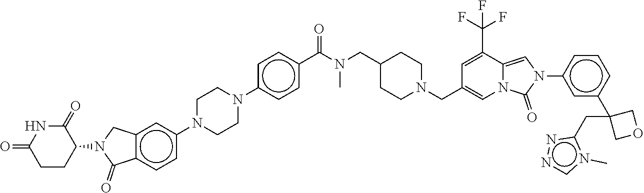 |
| 166 | 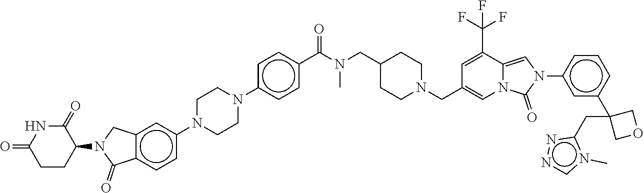 |
| 167 | 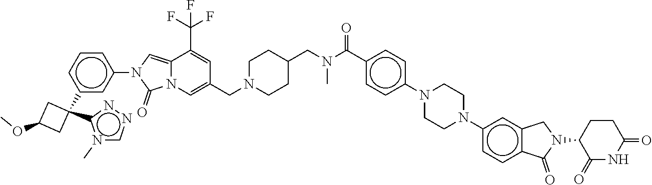 |
| 168 | 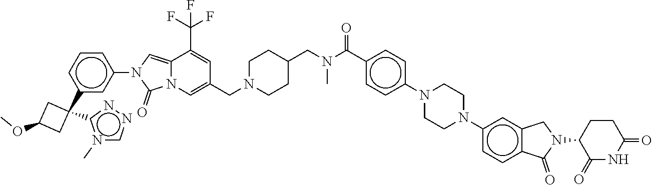 |
| 169 | 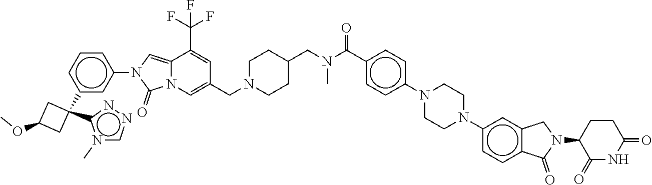 |
| 170 | 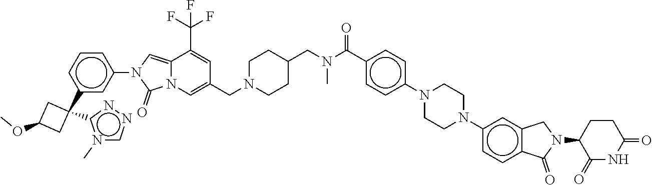 |
| 171 | 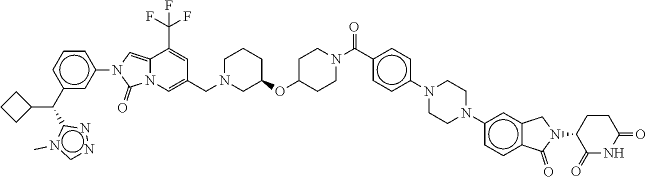 |
| 172 | 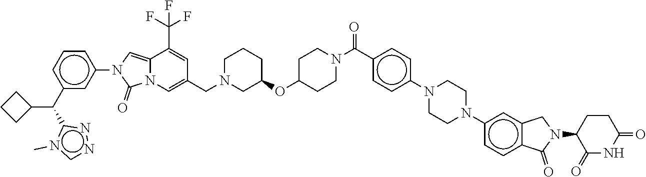 |
| 173 | 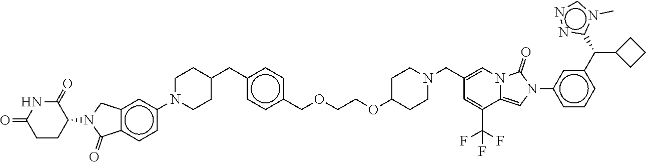 |
| 174 | 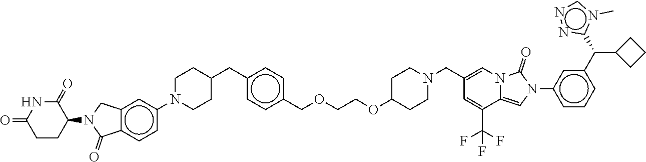 |
| 175 | 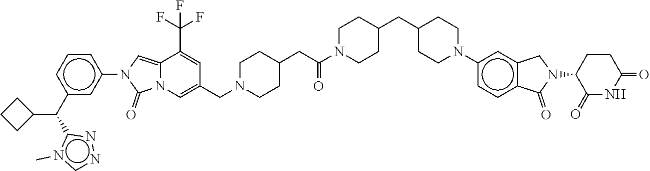 |
| 176 | 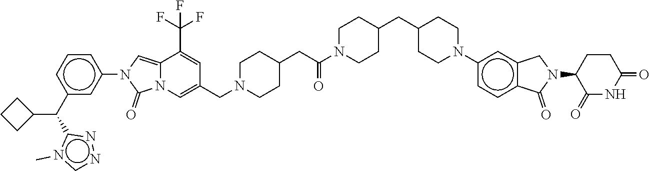 |
| 177 | 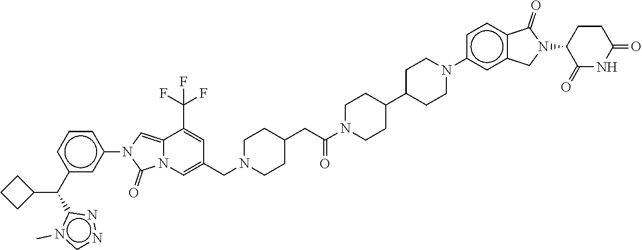 |
| 178 | 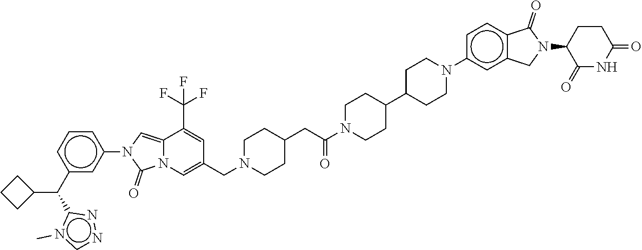 |
| 179 | 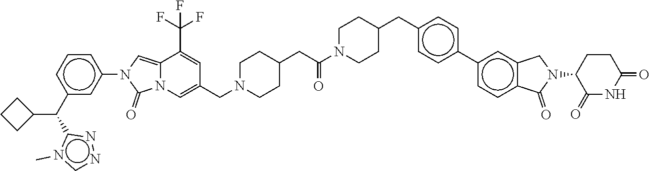 |
| 180 | 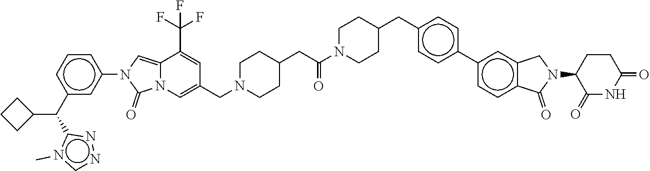 |
| 181 | 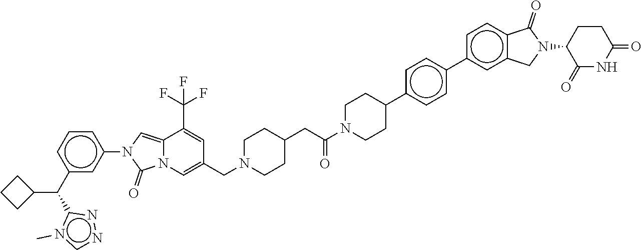 |
| 182 | 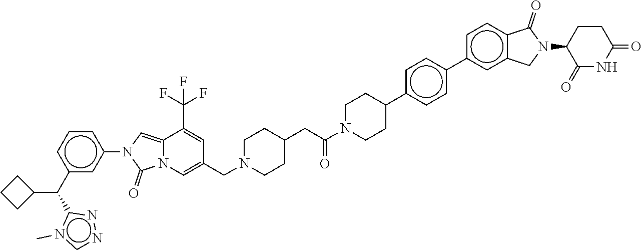 |
| 183 | 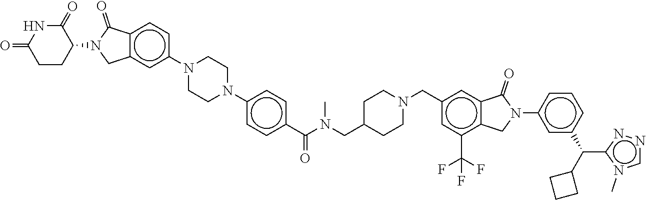 |
| 184 | 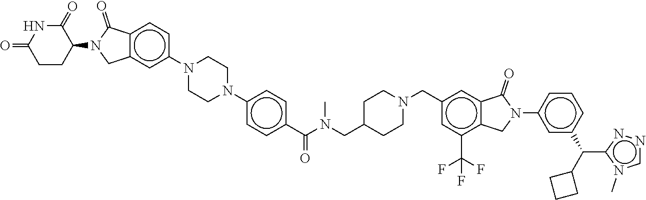 |
| 185 | 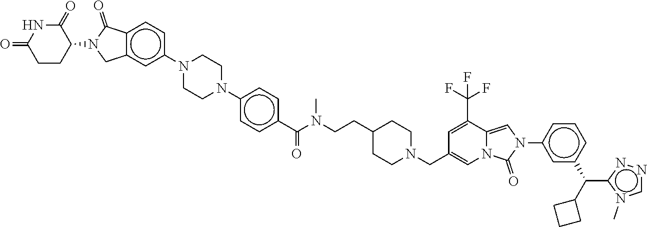 |
| 186 | 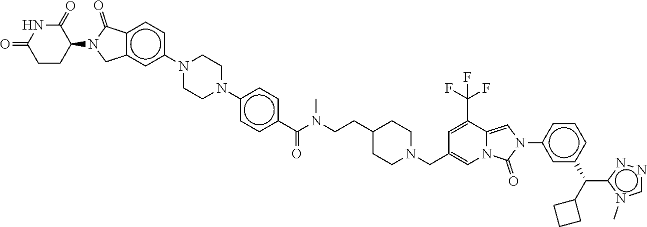 |
| 187 | 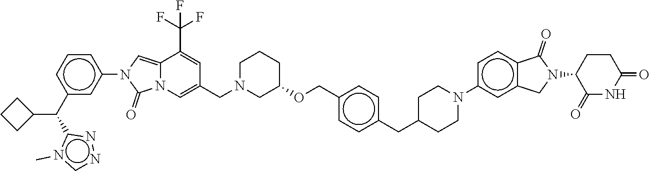 |
| 188 | 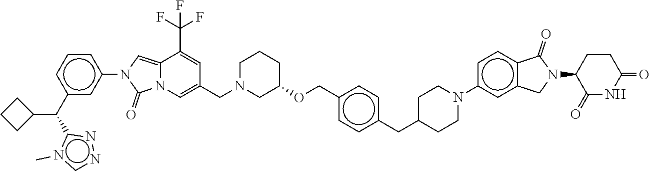 |
| 189 | 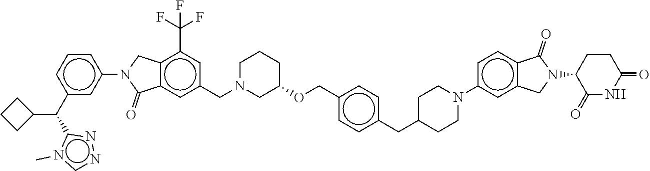 |
| 190 | 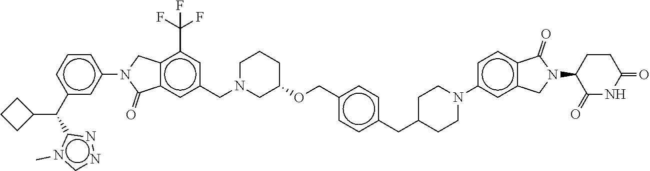 |
| 191 | 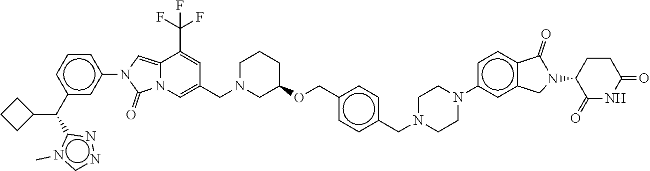 |
| 192 | 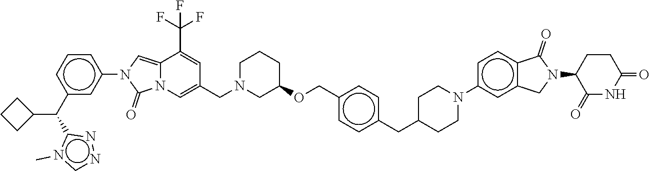 |
| 193 | 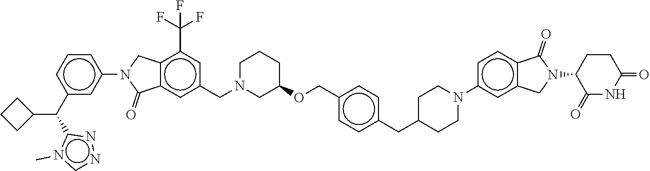 |
| 194 | 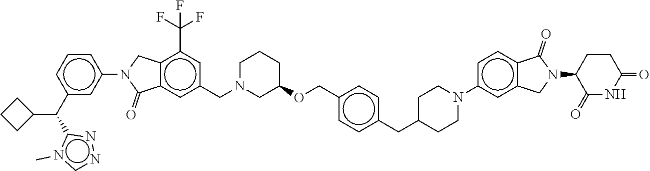 |
| 195 | 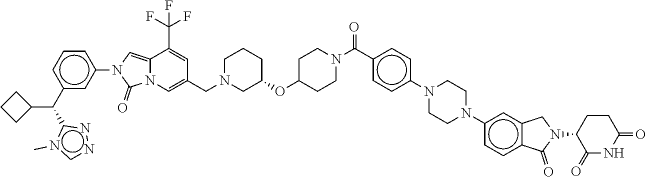 |
| 196 | 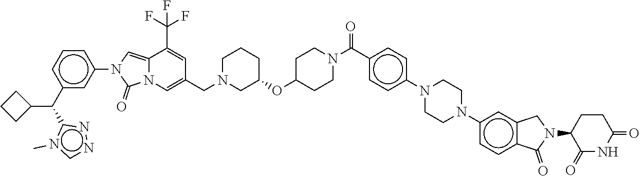 |
| 197 | 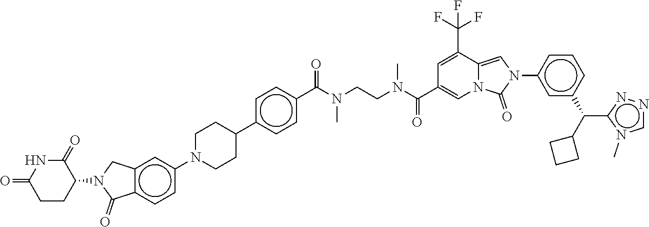 |
| 198 | 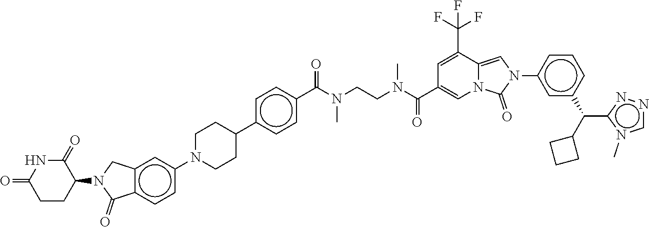 |
| 199 | 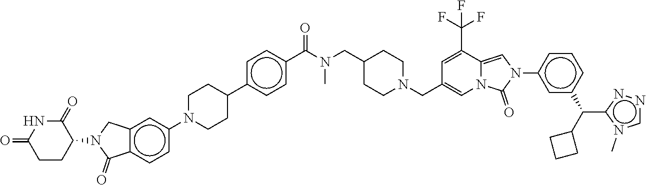 |
| 200 | 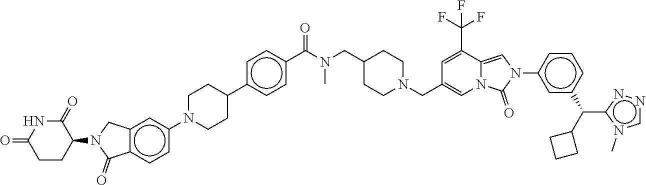 |
| 201 | 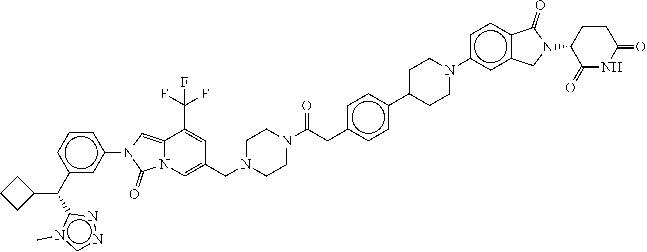 |
| 202 | 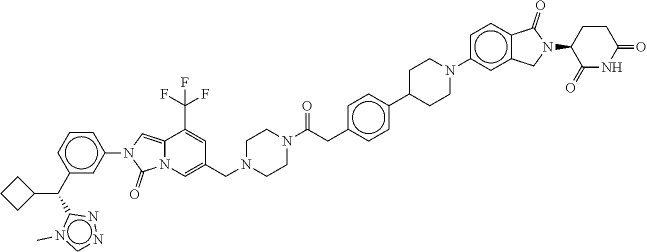 |
| 203 | 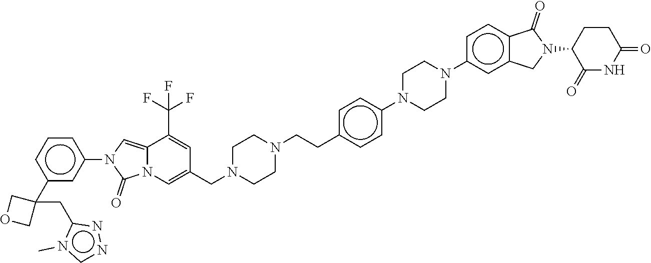 |
| 204 | 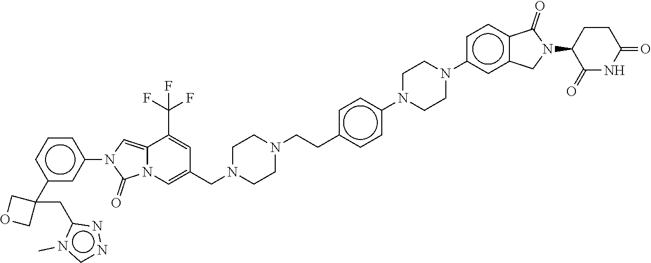 |
| 205 | 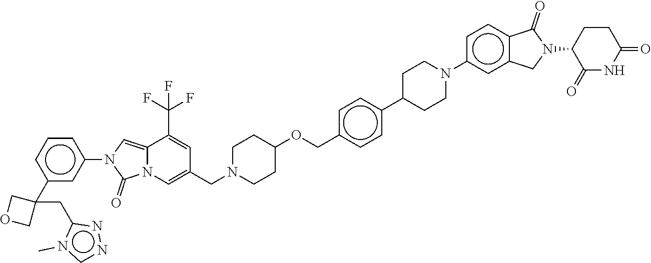 |
| 206 | 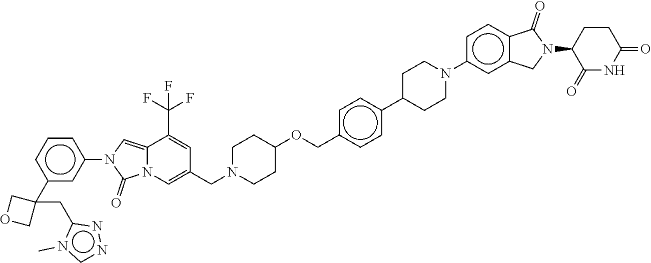 |
| 207 | 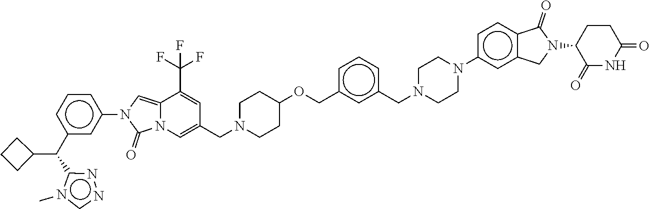 |
| 208 | 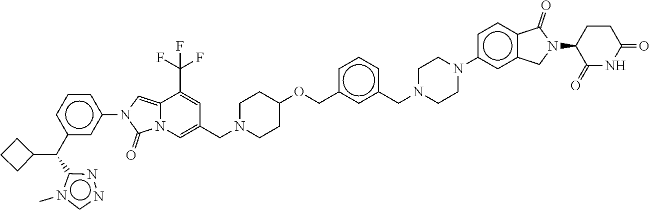 |
| 209 | 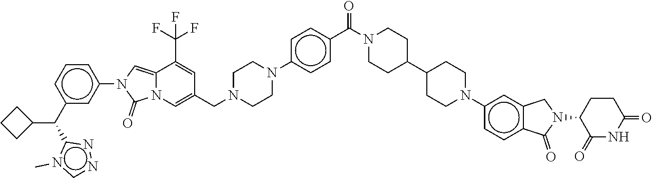 |
| 210 | 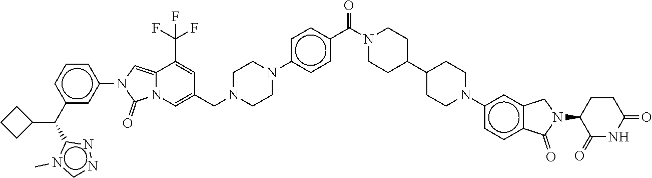 |
| 211 | 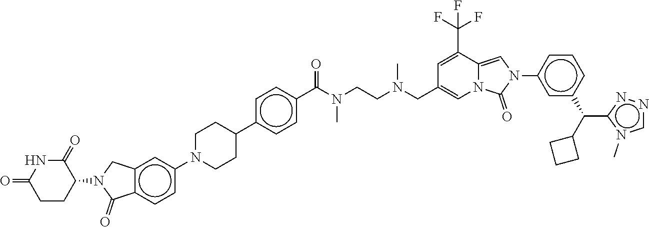 |
| 212 | 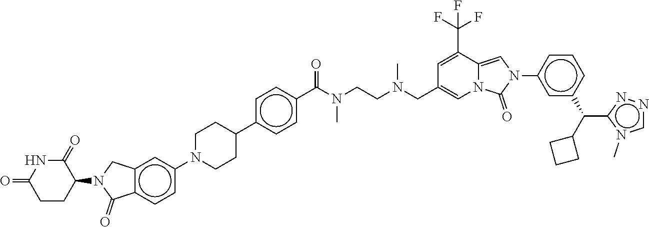 |
| 213 | 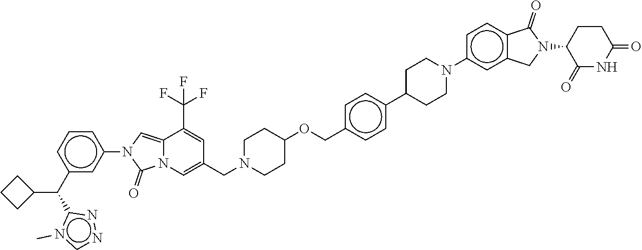 |
| 214 | 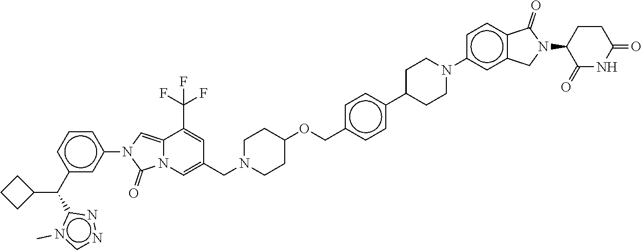 |
| 215 | 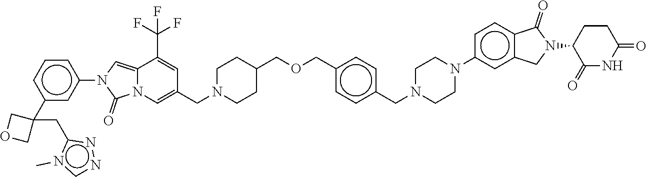 |
| 216 | 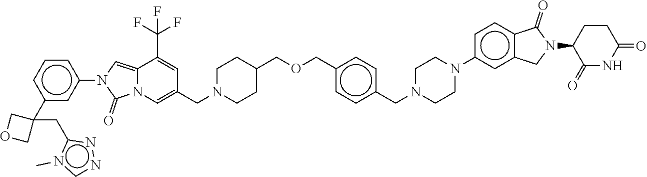 |
| 217 | 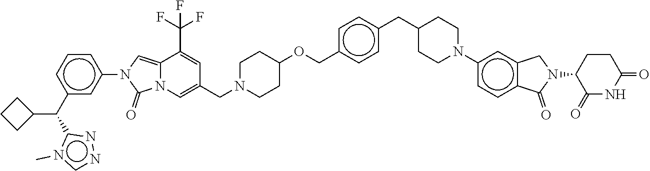 |
| 218 | 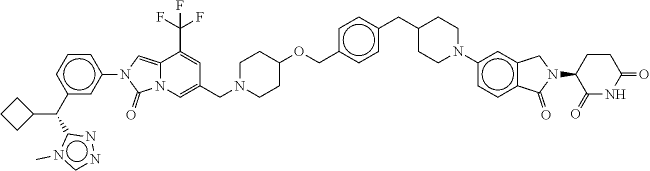 |
| 219 | 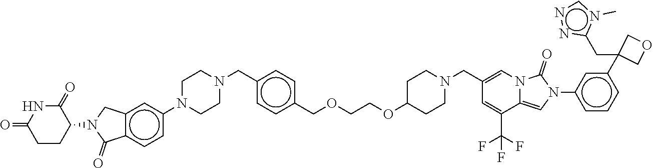 |
| 220 | 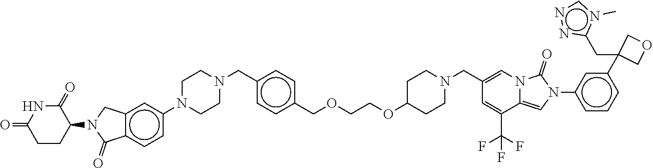 |
| 221 | 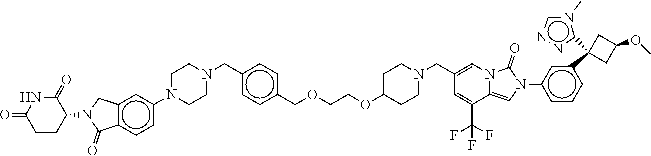 |
| 222 | 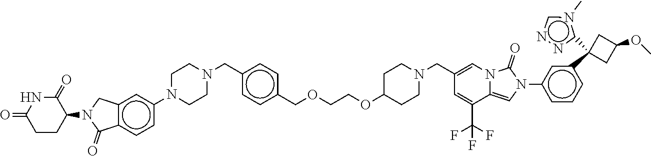 |
| 223 | 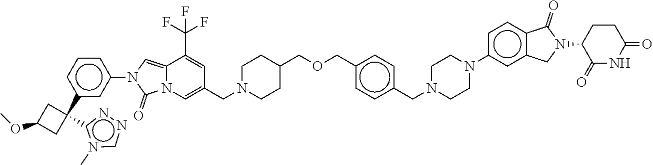 |
| 224 | 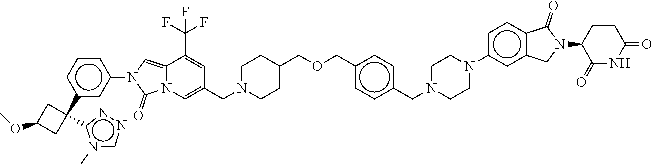 |
| 225 | 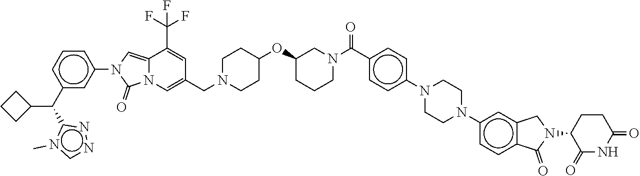 |
| 226 | 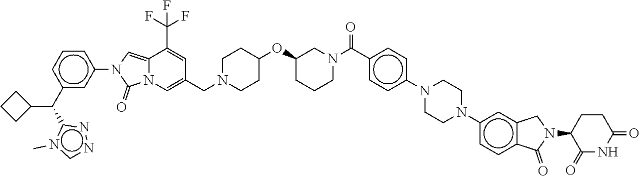 |
| 227 | 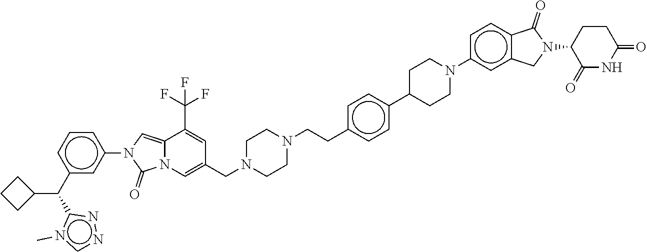 |
| 228 | 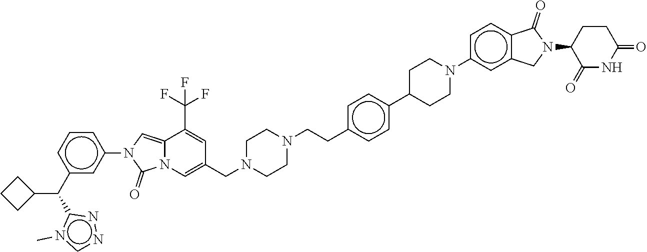 |
| 229 | 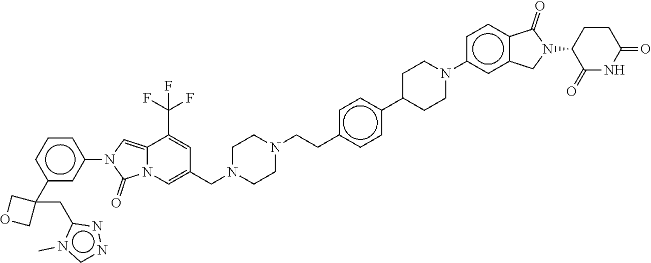 |
| 230 | 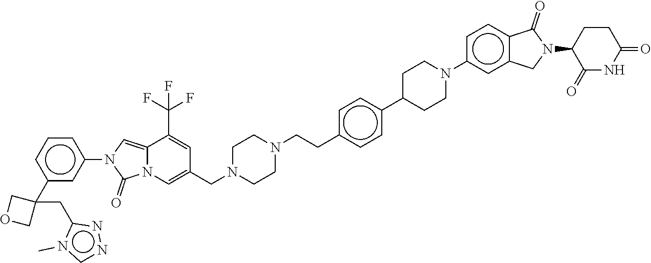 |
| 231 | 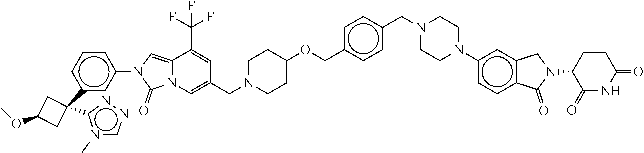 |
| 232 | 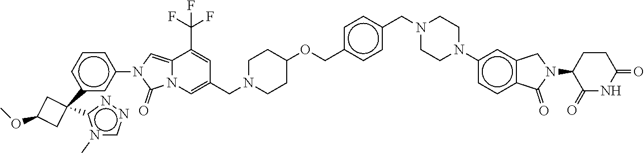 |
| 233 | 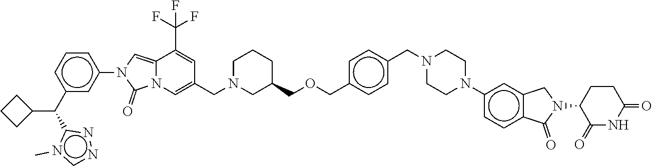 |
| 234 | 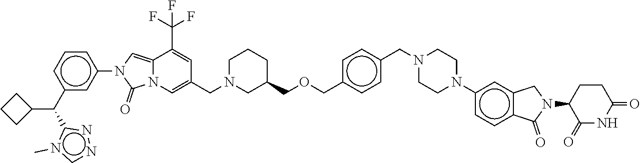 |
| 235 | 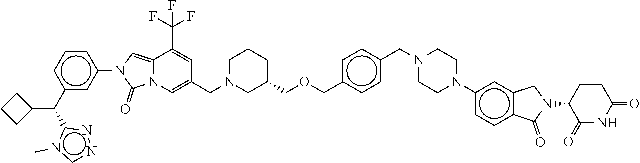 |
| 236 | 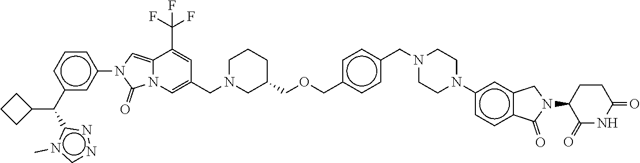 |
| 237 | 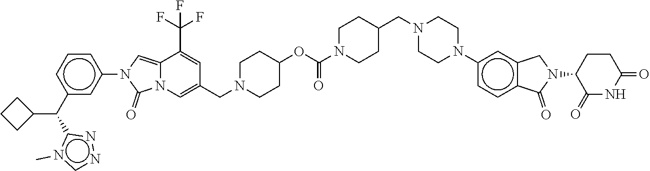 |
| 238 | 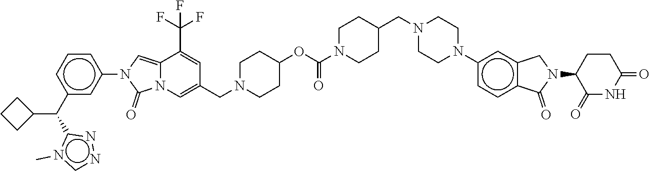 |
| 239 | 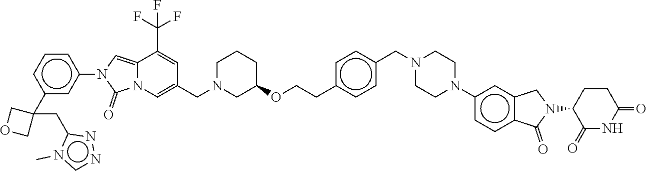 |
| 240 | 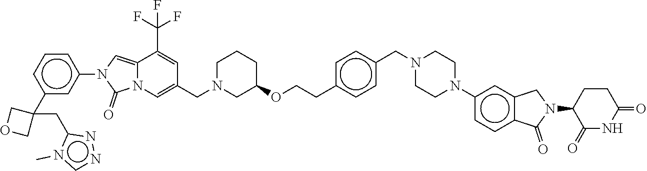 |
| 241 | 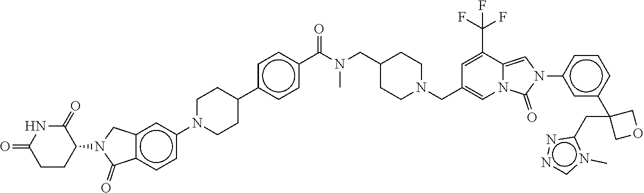 |
| 242 | 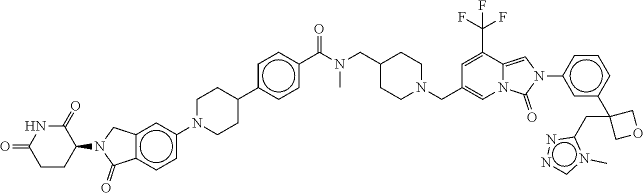 |
| 243 | 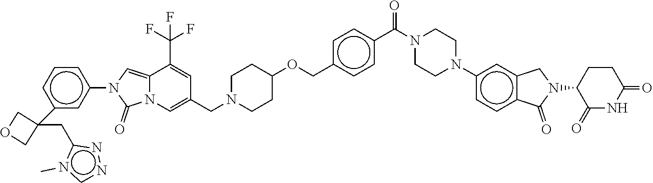 |
| 244 | 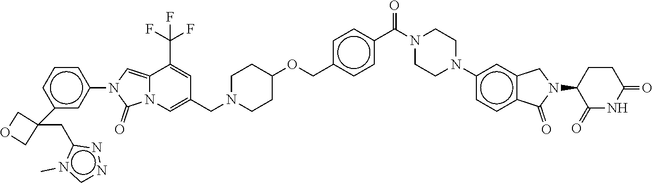 |
| 245 | 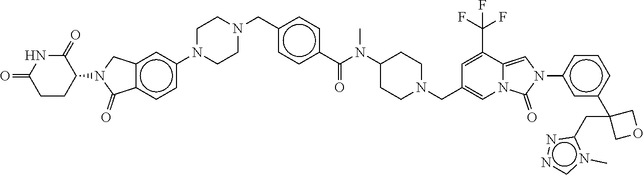 |
| 246 | 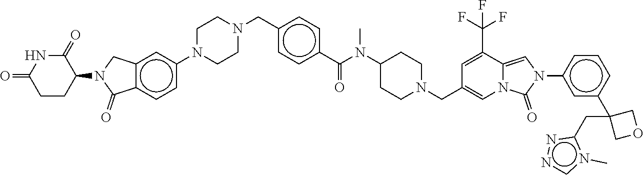 |
| 247 | 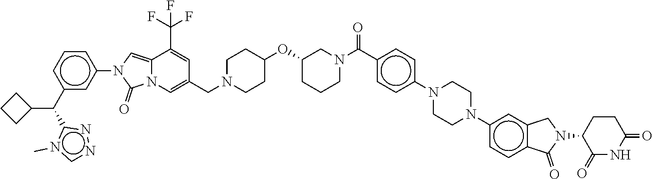 |
| 248 | 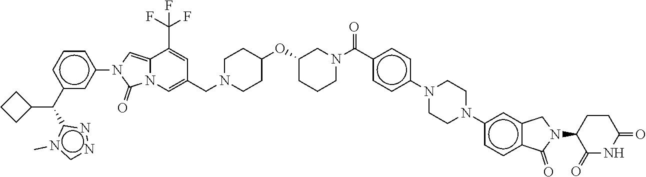 |
| 249 | 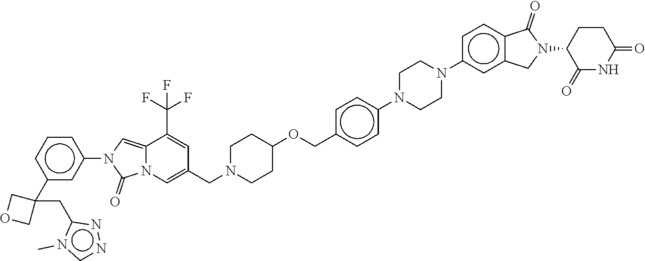 |
| 250 | 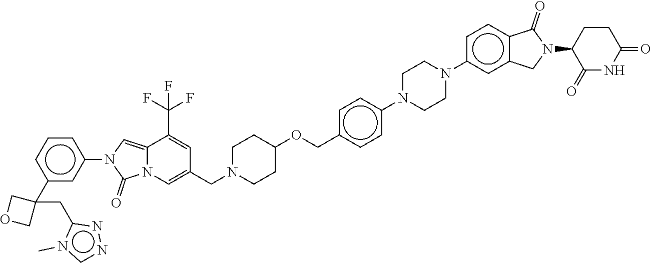 |
| 251 | 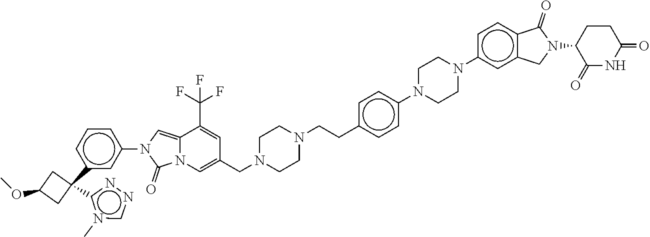 |
| 252 | 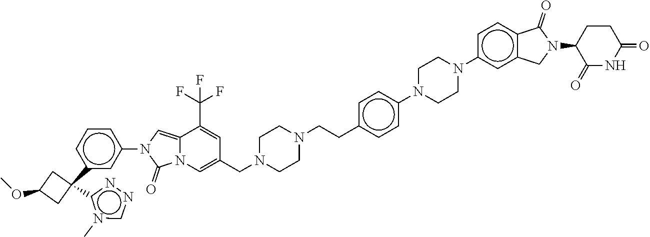 |
| 253 | 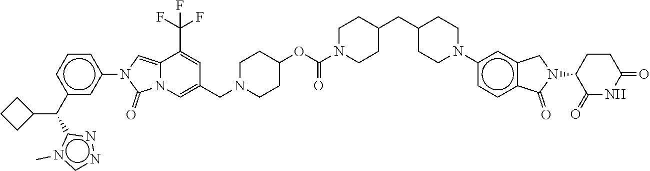 |
| 254 | 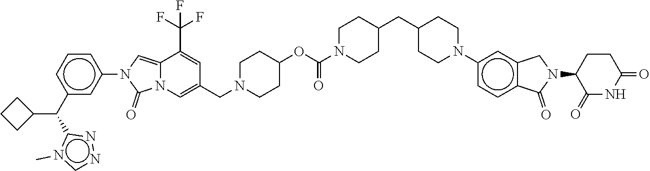 |
| 255 | 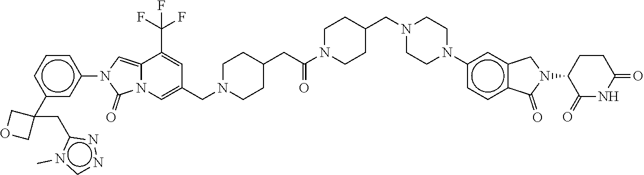 |
| 256 | 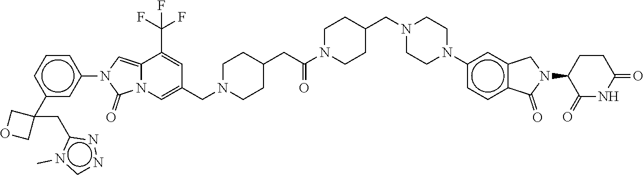 |
| 257 | 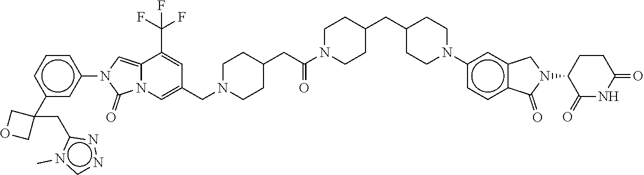 |
| 258 | 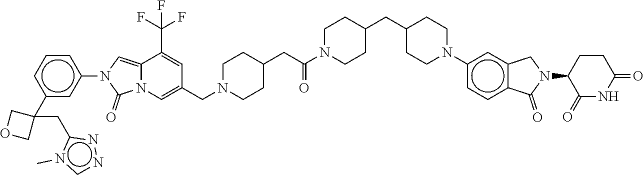 |
| 259 | 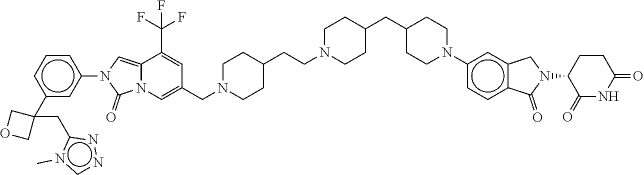 |
| 260 | 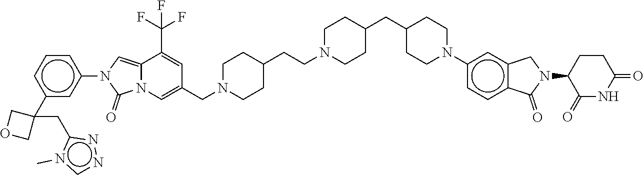 |
| 261 | 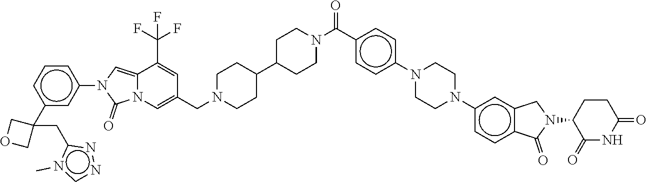 |
| 262 | 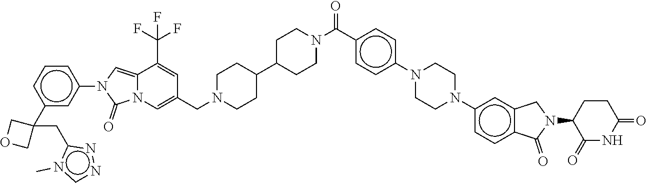 |
| 263 | 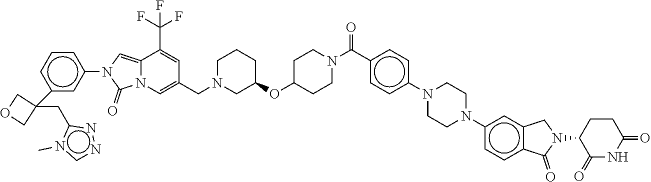 |
| 264 | 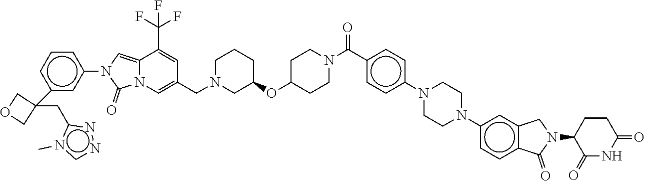 |

or a pharmaceutically acceptable salt thereof

A person of skill in the art will understand the present disclosure includes compounds with the stereochemistry which are the opposite of how they have been drawn. Additionally, the present disclosure contemplates tautomers of the compounds as drawn herein.

The present disclosure includes the racemate of any compound disclosed herein.

### Definitions

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “haloaliphatic” refers to an aliphatic group that is substituted with one or more halogen atoms.

The term “alkyl” refers to a straight or branched alkyl group. Exemplary alkyl groups are methyl, ethyl, propyl, isopropyl, butyl, isobutyl, and tert-butyl.

The term “haloalkyl” refers to a straight or branched alkyl group that is substituted with one or more halogen atoms.

The term “halogen” means F, Cl, Br, or I.

The term “aryl” used alone or as part of a larger moiety as in “aralkyl”, “aralkoxy”, or “aryloxyalkyl”, refers to monocyclic and bicyclic ring systems having a total of five to fourteen ring members, wherein at least one ring in the system is aromatic and wherein each ring in the system contains three to seven ring members. The term “aryl” may be used interchangeably with the term “aryl ring”. In certain embodiments of the present disclosure, “aryl” refers to an aromatic ring system which includes, but not limited to, phenyl, biphenyl, naphthyl, anthracyl and the like, which may bear one or more substituents. Also included within the scope of the term “aryl”, as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-”, used alone or as part of a larger moiety, e.g., “heteroaralkyl”, or “heteroaralkoxy”, refer to groups having 5 to 10 ring atoms, preferably 5, 6, or 9 ring atoms; having 6, 10, or 14π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring”, “heteroaryl group”, or “heteroaromatic”, any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle”, “heterocyclyl”, “heterocyclic radical”, and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in TV-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle”, “heterocyclyl”, “heterocyclyl ring”, “heterocyclic group”, “heterocyclic moiety”, and “heterocyclic radical”, are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)_{0- 4}N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Combinations of substituents and variables envisioned by this disclosure are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof. Examples of such purposes include, but are not limited to, blood transfusion, organ transplantation, biological specimen storage, and biological assays.

As used herein, a “therapeutically effective amount” means an amount of a substance (e.g., a therapeutic agent, composition, and/or formulation) that elicits a desired biological response. In some embodiments, a therapeutically effective amount of a substance is an amount that is sufficient, when administered as part of a dosing regimen to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of a provided compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. I

As used herein, the terms “treatment,” “treat,” and “treating” refer to partially or completely alleviating, inhibiting, delaying onset of, preventing, ameliorating and/or relieving a disorder or condition, or one or more symptoms of the disorder or condition, as described herein. In some embodiments, treatment may be administered after one or more symptoms have developed. In some embodiments, the term “treating” includes preventing or halting the progression of a disease or disorder. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence. Thus, in some embodiments, the term “treating” includes preventing relapse or recurrence of a disease or disorder.

The term “patient”, as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound(s) with which it is formulated. Pharmaceutically acceptable carriers, adjuvants or vehicles that may be used in the compositions of the compounds disclosed herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this disclosure that, upon administration to a recipient, is capable of providing, either directly or indirectly, a compound of this disclosure or an inhibitorily active metabolite or residue thereof.

The expression “dosage unit form” as used herein refers to a physically discrete unit of agent appropriate for the patient to be treated. It will be understood, however, that total daily usage of compounds and compositions of the present disclosure will be decided by the attending physician within the scope of sound medical judgment. Specific effective dose level for any particular patient or organism will depend upon a variety of factors including disorder being treated and severity of the disorder; activity of specific compound employed; specific composition employed; age, body weight, general health, sex and diet of the patient; time of administration, route of administration, and rate of excretion of a specific compound employed; duration of treatment; drugs used in combination or coincidental with a specific compound employed, and like factors well known in the medical arts.

### Alternative Embodiments

In an alternative embodiment, compounds described herein may also comprise one or more isotopic substitutions. For example, hydrogen may be ²H (D or deuterium) or ³H (T or tritium); carbon may be, for example, ¹³C or ¹⁴C; oxygen may be, for example, ¹⁸O; nitrogen may be, for example, ¹⁵N, and the like. In other embodiments, a particular isotope (e.g., ³H, ¹³C, ¹⁴C, ¹⁸O, or ¹⁵N) can represent at least 1%, at least 5%, at least 10%, at least 15%, at least 20%, at least 25%, at least 30%, at least 35%, at least 40%, at least 45%, at least 50%, at least 60%, at least 65%, at least 70%, at least 75%, at least 80%, at least 85%, at least 90%, at least 95%, at least 99%, or at least 99.9% of the total isotopic abundance of an element that occupies a specific site of the compound.

### Pharmaceutical Compositions

In some embodiments, the present disclosure provides a composition comprising a compound of Formula (I) and a pharmaceutically acceptable carrier, adjuvant, or vehicle. In some embodiments, the amount of compound in compositions contemplated herein is such that is effective to measurably treat a disease or disorder in a biological sample or in a patient. In certain embodiments, the amount of compound in compositions of this disclosure is such that is effective to measurably treat a disease or disorder in a biological sample or in a patient. In certain embodiments, a composition contemplated by this disclosure is formulated for administration to a patient in need of such composition. In some embodiments, a composition contemplated by this disclosure is formulated for oral administration to a patient.

In some embodiments, compositions of the present disclosure may be administered orally, parenterally, by inhalation spray, topically, rectally, nasally, buccally, vaginally or via an implanted reservoir. In some preferred embodiments, compositions are administered orally, intraperitoneally or intravenously. In some embodiments, sterile injectable forms of the compositions comprising one or more compounds of Formula (I) may be aqueous or oleaginous suspension. In some embodiments, suspensions may be formulated according to techniques known in the art using suitable dispersing or wetting agents and suspending agents. In some embodiments, sterile injectable preparation may also be a sterile injectable solution or suspension in a non-toxic parenterally acceptable diluent or solvent, for example as a solution in 1,3-butanediol. In some embodiments, among the acceptable vehicles and solvents that may be employed are water, Ringer's solution and isotonic sodium chloride solution. In some embodiments, additional examples include, but are not limited to, sterile, fixed oils are conventionally employed as a solvent or suspending medium.

The term “parenteral” as used herein includes subcutaneous, intravenous, intramuscular, intra-articular, intra-synovial, intrasternal, intrathecal, intrahepatic, intralesional and intracranial injection or infusion techniques.

Pharmaceutically acceptable compositions comprising one or more compounds of Formula (I) may be orally administered in any orally acceptable dosage form including, but not limited to, capsules, tablets, aqueous suspensions or solutions. In some embodiments, carriers used include lactose and corn starch. Lubricating agents, such as magnesium stearate, are also typically added. In some embodiments, useful diluents include lactose and dried cornstarch. In some embodiments, when aqueous suspensions are required for oral use, an active ingredient is combined with emulsifying and suspending agents. In some embodiments, certain sweetening, flavoring or coloring agents may also be added.

Alternatively, pharmaceutically acceptable compositions comprising a compound of Formula (I) may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions comprising a compound of Formula (I) may also be administered topically, especially when the target of treatment includes areas or organs readily accessible by topical application, including diseases of the eye, the skin, or the lower intestinal tract. Suitable topical formulations are readily prepared for each of these areas or organs. In some embodiments, pharmaceutically acceptable compositions may be formulated in a suitable ointment containing the active component suspended or dissolved in one or more carriers. Carriers for topical administration of compounds of this disclosure include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

Pharmaceutically acceptable compositions comprising a compound of Formula (I) may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

In some embodiments, an amount of a compound of the present disclosure that may be combined with the carrier materials to produce a composition in a single dosage form will vary depending upon the host treated, the particular mode of administration. Preferably, provided compositions should be formulated so that a dosage of between 0.01-100 mg/kg body weight/day of the inhibitor can be administered to a patient receiving these compositions.

### Methods of Using Compounds of the Present Disclosure

In some embodiments, the present disclosure provides a method for treating or lessening the severity of a disease or condition associated with cell proliferation in a patient comprising the step of administering to said patient a composition according to the present disclosure.

The term “disease or condition associated with cell proliferation”, as used herein means any disease or other deleterious condition in which cell proliferation is known to play a role. Accordingly, another embodiment of the present disclosure relates to treating or lessening the severity of one or more diseases in which cell proliferation is known to play a role. In some embodiments, a disease or condition associated with cell proliferation is hyperplasia or cancer. In some embodiments, a disease or condition associated with cell proliferation is cancer.

In some embodiments, administration of a compound of the present disclosure results in arrest of mitosis.

In some embodiments, administration of a compound of the present disclosure results in arrest of mitosis. In some embodiments, mitotic arrest is defined as a 10-100% reduction in mitosis. In some embodiments, mitotic arrest is defined as a 20-100% reduction in mitosis. In some embodiments, mitotic arrest is defined as a 30-100% reduction in mitosis. In some embodiments, mitotic arrest is defined as a 40-100% reduction in mitosis. In some embodiments, mitotic arrest is defined as a 50-100% reduction in mitosis. In some embodiments, mitotic arrest is defined as a 60-100% reduction in mitosis. In some embodiments, mitotic arrest is defined as a 70-100% reduction in mitosis. In some embodiments, mitotic arrest is defined as a 80-100% reduction in mitosis. In some embodiments, mitotic arrest is defined as a 90-100% reduction in mitosis. In some embodiments, mitotic arrest is defined as a 100% reduction in mitosis.

In some embodiments, compounds and compositions, according to a method of the present disclosure, may be administered using any amount and any route of administration effective for treating or lessening the severity of cancer. The exact amount required will vary from subject to subject, depending on the species, age, and general condition of the subject, severity of the infection, particular agent, its mode of administration, and the like. Compounds of the present disclosure are preferably formulated in dosage unit form for ease of administration and uniformity of dosage.

In some embodiments, cancer is a hematologic cancer. In some embodiments, a hematologic cancer is selected from a group consisting of lymphoma, leukemia, and myeloma. In some embodiments, a hematologic cancer is lymphoma. In some embodiments, a hematologic cancer is leukemia. In some embodiments, a hematologic cancer is myeloma.

In some embodiments, cancer is a non-hematologic cancer. In some embodiments, a non-hematologic cancer is a sarcoma or a carcinoma. In some embodiments, a non-hematologic cancer is a sarcoma. In some embodiments, a non-hematologic cancer is carcinoma.

In some embodiments, a subject has one or more of increased T-cell activation, increased T-cell proliferation, decreased T-cell exhaustion, decreased T-cell anergy and decreased T-cell tolerance after administration of compound of the present disclosure. In some embodiments, administration of a compound of the present disclosure to a subject in need there of results in one or more of increased T-cell activation, increased T-cell proliferation, decreased T-cell exhaustion, decreased T-cell anergy and decreased T-cell tolerance.

In some embodiments, a subject has increased NK-cell activation. In some embodiments, increased NK-cell activation comprises increased production of cytokines.

In some embodiments, pharmaceutically acceptable compositions of comprising compounds of the present disclosure can be administered to humans and other animals orally, rectally, parenterally, intracisternally, intravaginally, intraperitoneally, topically (as by powders, ointments, or drops), buccally, as an oral or nasal spray, or the like, depending on the severity of infection being treated. In certain embodiments, compounds of the present disclose may be administered orally or parenterally at dosage levels of about 0.01 mg/kg to about 50 mg/kg and preferably from about 1 mg/kg to about 25 mg/kg, of subject body weight per day, one or more times a day, to obtain desired therapeutic effect.

In some embodiments, one or more additional therapeutic agents, may also be administered in combination with compounds of the present disclosure. In some embodiments, a compound of the present disclosure and one or more additional therapeutic agents may be administered as part of a multiple dosage regime. In some embodiments, a compound of the present disclosure and one or more additional therapeutic agents may be administered may be administered simultaneously, sequentially or within a period of time. In some embodiments, a compound of the present disclosure and one or more additional therapeutic agents may be administered within five hours of one another. In some embodiments, a compound of the present disclosure and one or more additional therapeutic agents may be administered within 24 hours of one another. In some embodiments, a compound of the present disclosure and one or more additional therapeutic agents may be administered within one week of one another.

In some embodiments, a compound of the present disclosure and one or more additional therapeutic agents may be formulated into a single dosage form.

### EXEMPLIFICATION

### Preparation of Intermediate A

### Synthesis of Intermediate A1.

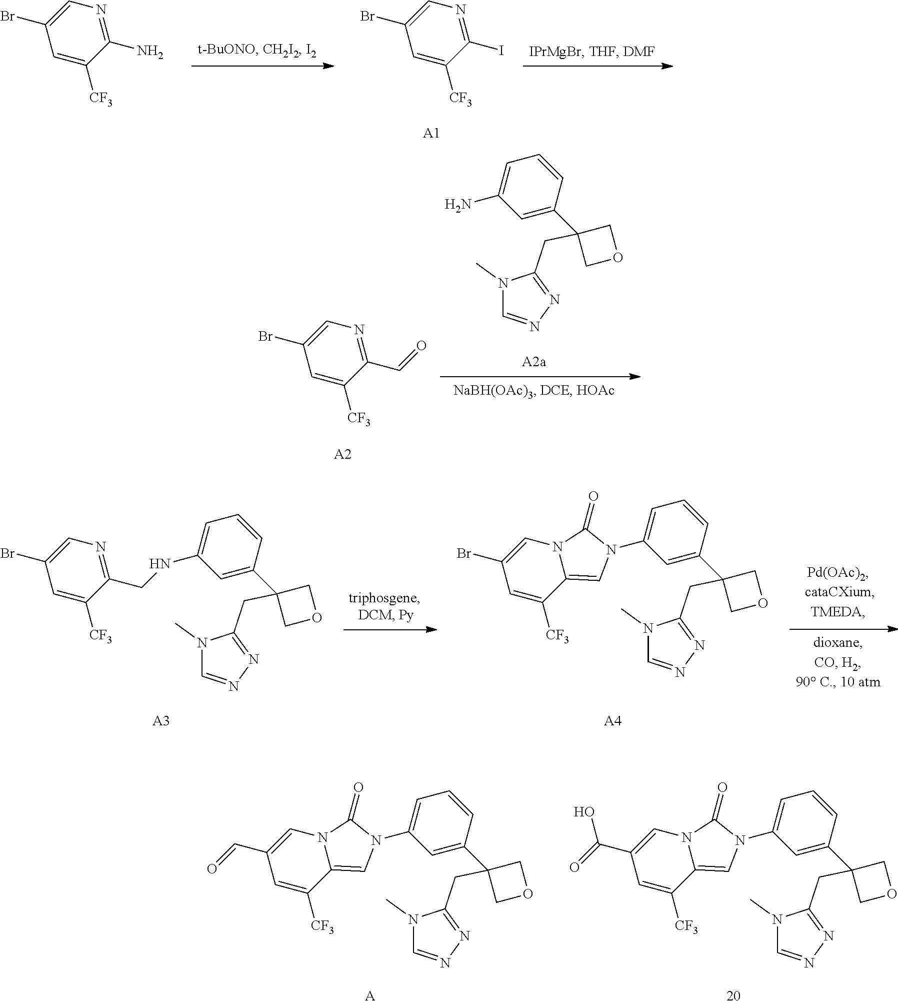

To a stirred mixture of 5-bromo-3-(trifluoromethyl)pyridin-2-amine (25.00 g, 103.730 mmol, 1.00 equiv) in CH₂I₂ (75.00 mL) was added t-BuNO₂ (12.84 g, 124.515 mmol, 1.20 equiv) dropwise. The resulting mixture was stirred for 1 h at room temperature. To the above mixture was added I₂ (28.96 g, 114.102 mmol, 1.10 equiv) in portions. The resulting mixture was stirred for additional 6 h at room temperature. The reaction was quenched by the addition of NaHCO₃ (aq.) (300 mL). The aqueous layer was extracted with EtOAc (2×200 mL). The resulting mixture was concentrated under reduced pressure. The residue was purified by silica gel column chromatography, eluted with PE/EtOAc (50:1) to afford Intermediate A1 (17 g, 46.57%) as a light yellow oil.

### Synthesis of Intermediate A2.

To a stirred solution of Intermediate A1 (17.00 g, 48.310 mmol, 1.00 equiv) in THF (300.00 mL) was added i-PrMgBr (18.32 mL, 53.141 mmol, 1.10 equiv) dropwise at −78° C. under argon atmosphere. The resulting mixture was stirred for 30 min at −78° C. under argon atmosphere. To the above mixture was added DMF (7.06 g, 96.588 mmol, 2.00 equiv) dropwise at −78° C. The resulting mixture was stirred for additional 2 hours at −78° C. The reaction was quenched by the addition of NH₄Cl (aq.) (800 mL) at room temperature. The aqueous layer was extracted with EtOAc (2×400 mL). The resulting mixture was concentrated under reduced pressure. The residue was purified by silica gel column chromatography, eluted with PE/EtOAc (20:1) to afford Intermediate A2 (4.5 g, 36.67%) as a white solid.

### Synthesis of Intermediate A3.

To a stirred solution of Intermediate A2 (3.50 g, 13.779 mmol, 1.00 equiv) and Compound A2a (3.37 g, 13.795 mmol, 1.00 equiv) in DCE (50.00 mL) were added HOAc (1.65 g, 27.558 mmol, 2.00 equiv) and NaBH(OAc)₃ (5.84 g, 27.558 mmol, 2.00 equiv). The resulting mixture was stirred overnight at room temperature. The resulting mixture was diluted with water (100 mL). The aqueous layer was extracted with CH₂Cl₂ (3×150 mL). The resulting mixture was concentrated under vacuum. The residue was purified by silica gel column chromatography, eluted with CH₂Cl₂/MeOH (50:1) to afford intermediate A3 (4.5 g, 67.71%) as a light yellow solid.

### Synthesis of Intermediate A4.

To a stirred solution of Intermediate A3 (4.40 g, 9.123 mmol, 1.00 equiv) and pyridine (4.33 g, 54.741 mmol, 6.00 equiv) in DCM (250.00 mL) was added triphosgene (0.95 g, 3.193 mmol, 0.35 equiv) at 0° C. The resulting mixture was stirred 1 h at room temperature. The reaction was quenched by the addition of NaHCO₃ (aq.) (100 mL). The aqueous layer was extracted with CH₂Cl₂/MeOH=10/1 (3×200 mL). The resulting mixture was concentrated under vacuum. The residue was purified by trituration with methyl tert-butyl ether (50 mL). This resulted in Intermediate A4 (4.2 g, 90.57%) as a yellow solid.

### Synthesis of Intermediate A.

To a solution of Intermediate A4 (2.20 g, 4.328 mmol, 1.00 equiv), TMEDA (0.50 g, 4.328 mmol, 1.00 equiv) in dioxane (180.00 mL) was added butyldi-1-adamantylphosphine (0.31 g, 0.866 mmol, 0.20 equiv) and Pd(OAc)₂ (0.10 g, 0.433 mmol, 0.10 equiv) in an autoclave. After flushing the autoclave three times with CO/H₂ (1:1), the mixture was pressurized to 10 atm with CO/H₂ (1:1) at 90° C. and stirred overnight. The resulting mixture was concentrated under vacuum. The residue was purified by silica gel column chromatography, eluted with CH₂Cl₂/MeOH (20:1) to afford Intermediate A (1.1 g, 55.56%) as a yellow solid.

### Preparation of Intermediate B

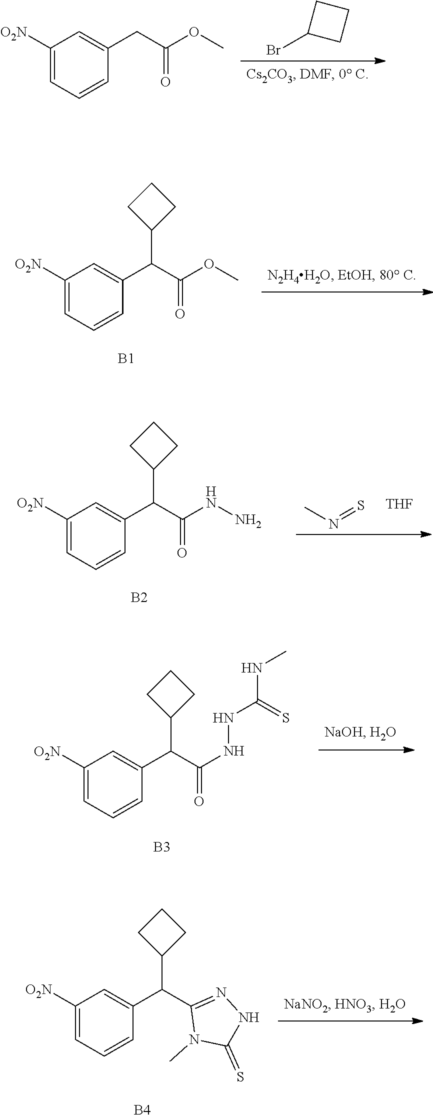

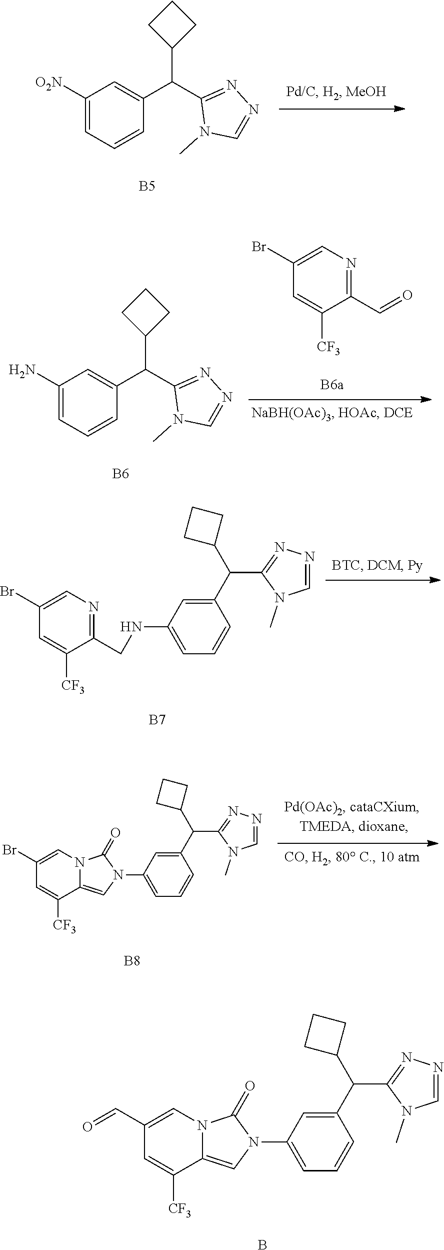

### Synthesis of Intermediate B1.

A mixture of methyl 2-(3-nitrophenyl)acetate (48.1 g, 246.447 mmol, 1 equiv) and Cs₂CO₃ (401.49 g, 1232.235 mmol, 5 equiv) in DMF (500 mL) was stirred for 3 h at 0° C. under nitrogen atmosphere. To the above mixture was added bromocyclobutane (99.81 g, 739.341 mmol, 3 equiv) at room temperature. The resulting mixture was stirred overnight at room temperature. The reaction was diluted with NH₄Cl (aq.) (3 L) at 0° C. The aqueous layer was extracted with EtOAc (3×500 mL). The resulting mixture was concentrated under reduced pressure. The residue was purified by silica gel column chromatography, eluted with PE/EA (150:1) to afford Intermediate B1 (49 g, 73.38%) as an off-white solid.

### Synthesis of Intermediate B2.

To a stirred solution of Intermediate B1 (49 g, 196.577 mmol, 1 equiv) in EtOH (500 mL) was added hydrazine hydrate (98%) (251.04 g, 4914.425 mmol, 25 equiv, 98%) at room temperature. The resulting mixture was stirred overnight at 80° C. The reaction was diluted by the addition of water (500 mL) at room temperature. The aqueous layer was extracted with CH₂Cl₂/MEOH (10:1) (3×500 mL). The resulting mixture was concentrated under reduced pressure. The residue was purified by silica gel column chromatography, eluted with CH₂Cl₂/MeOH (100:1) to afford Intermediate B2 (43 g, 79.86%) as a yellow oil.

### Synthesis of Intermediate B3.

To a stirred solution of Intermediate B2 (45 g, 180.527 mmol, 1 equiv) in THF (450 mL) was added methyl isothiocyanate (33.00 g, 451.317 mmol, 2.5 equiv) at room temperature under nitrogen atmosphere. The resulting mixture was stirred for 3 h at room temperature. The resulting mixture was diluted with water (280 mL). The resulting mixture was filtered, the filter cake was washed with water (3×50 mL). The resulting solid was dried under vacuum. This resulted in Intermediate B3 (55 g, 86.00%) as a white solid.

### Synthesis of Intermediate B4.

To a stirred solution of NaOH (66 g, 1650.120 mmol, 9.67 equiv) in H₂O (1.65 L) was added Intermediate B3 (55 g, 170.606 mmol, 1 equiv) at room temperature. The resulting mixture was stirred overnight at room temperature. The mixture was acidified to pH 5 with HCl (1 M). The resulting mixture was filtered, the filter cake was washed with water (3×50 mL). The resulting solid was dried under vacuum. This resulted in Intermediate B4 (50 g, 86.66%) as a off-white solid.

### Synthesis of Intermediate B5.

To a stirred mixture of Intermediate B4 (50 g, 164.274 mmol, 1 equiv) in EtOAc (190 mL) and H₂O (760 mL) was added NaNO₂ (113.3 g 1642.74 mmol, 10 equiv) at room temperature. To the above mixture was added HNO₃ (1642 mL, 1642.74 mmol, 10.00 equiv, 1 M) dropwise at 0 degrees C. The resulting mixture was stirred overnight at room temperature. The mixture was neutralized to pH 7 with saturated NaHCO₃ (aq.). The aqueous layer was extracted with CH₂Cl₂/MeOH (10:1) (3×500 mL). The resulting mixture was concentrated under reduced pressure. The residue was purified by silica gel column chromatography, eluted with CH₂Cl₂/MeOH (50:1) to afford Intermediate B5 (40 g, 85.84%) as a yellow solid.

### Synthesis of Intermediate B6.

To a solution of Intermediate B5 (40 g, 146.892 mmol, 1 equiv) in 1.2 L MeOH was added Pd/C (20%, 8 g) in a 2 L round-bottom flask. The mixture was hydrogenated at room temperature overnight under hydrogen atmosphere using a hydrogen balloon, filtered through a Celite pad, and concentrated under reduced pressure. This resulted in Intermediate B6 (35 g, 94.39%) as an off-white solid.

### Synthesis of Intermediate B7.

To a stirred solution of Intermediate B6 (31.45 g, 123.800 mmol, 1.2 equiv) and Compound B6a (31.45 g, 123.800 mmol, 1.2 equiv) in DCE (300 mL) were added NaBH(OAc)₃ (43.73 g, 206.334 mmol, 2 equiv) and HOAc (6.20 g, 103.167 mmol, 1 equiv) at room temperature under nitrogen atmosphere. The resulting mixture was stirred overnight at room temperature under nitrogen atmosphere. The reaction was quenched by the addition of water (500 mL) at room temperature. The aqueous layer was extracted with EtOAc (3×500 mL), dried over anhydrous Na₂SO₄. After filtration, the filtrate was concentrated under reduced pressure. The residue was purified by trituration with MTBE (2×50 mL). This resulted in Intermediate B7 (34 g, 65.18%) as a white solid.

### Synthesis of Intermediate B8.

To a stirred solution of Intermediate B7 (34 g, 70.784 mmol, 1 equiv) and pyridine (33.59 g, 424.704 mmol, 6 equiv) in DCM (400 mL) were added Triphosgene (7.35 g, 24.774 mmol, 0.35 equiv) dropwise at 0° C. under nitrogen atmosphere. The resulting mixture was stirred for 10 min at room temperature under nitrogen atmosphere. The reaction was quenched by the addition of water (500 mL) at room temperature. The resulting mixture was extracted with CH₂Cl₂ (3×500 mL) and dried over anhydrous CaCl₂. After filtration, the filtrate was concentrated under reduced pressure. The residue was purified by trituration with MTBE (2×100 mL) to give Intermediate B8 (33 g, 87.47%) as a yellow solid.

### Synthesis of Intermediate B.

To a solution of Intermediate B8 (33 g, 65.175 mmol, 1 equiv) and TMEDA (15.15 g, 130.350 mmol, 2 equiv) in dioxane (1000 mL) was added bis(adamantan-1-yl)(butyl)phosphane (4.67 g, 13.035 mmol, 0.2 equiv) and Pd(OAc)₂ (1.46 g, 6.518 mmol, 0.1 equiv) in an autoclave. After flushing the autoclave three times with CO/H₂ (1:1), the mixture was pressurized to 10 atm with CO/H₂ (1:1) and run overnight at 80 degrees C. The resulting mixture was concentrated under vacuum. The residue was purified by silica gel column chromatography, eluted with CH₂Cl₂/MeOH (20:1) to CH₂Cl₂/MeOH (5:1) to afford Intermediate B (20 g, 67.38%) as a yellow solid.

### Preparation of Intermediate C

### Synthesis of Intermediate C1.

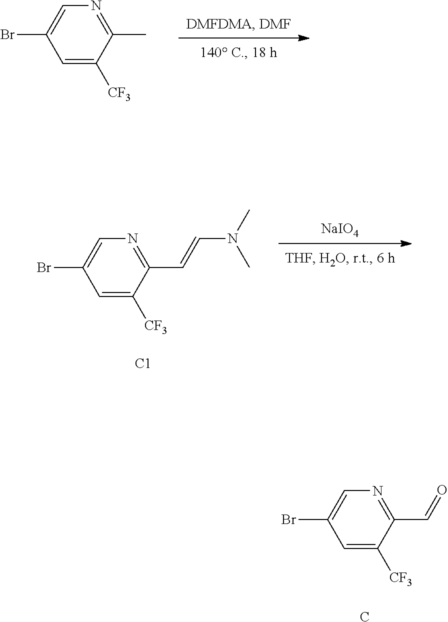

### Synthesis of Intermediate C1.

A mixture of 5-bromo-2-methyl-3-(trifluoromethyl)pyridine (25 g, 104.16 mmol, 1 eq) in DMF (300 mL) and DMF-DMA (269.10 g, 2.26 mol, 300 mL) was stirred at 140° C. for 18 hr. The reaction mixture was concentrated in vacuum to afford intermediate C1 (30 g, crude) as a brown oil, which was used directly without further purification.

### Synthesis of Intermediate C.

To a solution of Intermediate C1 (30 g, 101 mmol) in THF (150 mL) and water (150 mL) was added NaIO4 (65.2 g, 304 mmol). The mixture was stirred at 20° C. for 6 hr. The reaction mixture was filtered and the filter cake was washed with ethyl acetate (200 mL). The filtrate was washed with saturated aqueous sodium bicarbonate solution (200 mL) and saturated aqueous brine solution (200 mL), dried over anhydrous sodium sulfate, filtered, and concentrated in vacuum. The residue was purified by normal phase SiO2 chromatography (0-20% EtOAc/petroleum ether) to afford Intermediate C (8 g, 30.9% yield) as a brown oil.

### Preparation of Intermediate D

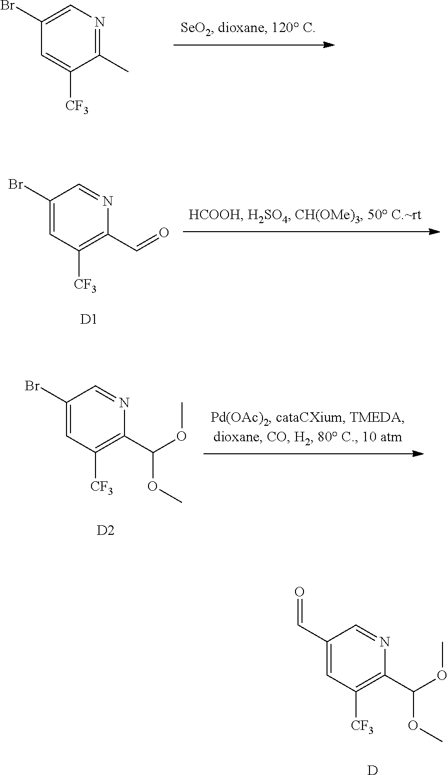

### Synthesis of Intermediate Intermediate D1.

A solution of 5-bromo-2-methyl-3-(trifluoromethyl)pyridine (60 g, 249.976 mmol, 1 equiv) in dioxane (350 mL) was added SeO2 (69.35 g, 624.940 mmol, 2.5 equiv). The resulting mixture was stirred overnight at 120 degrees C. The resulting mixture was filtered, the filter cake was washed with EtOAc (3×50 mL). The filtrate was diluted with water (300 mL). The aqueous layer was extracted with EtOAc (3×100 mL). The residue was purified by silica gel column chromatography, eluted with PE/EA (50:1) to afford Intermediate D1 (49 g, 69.45%) as a yellow oil.

### Synthesis of Intermediate D2.

Into a 250 mL round-bottom flask were added Intermediate D1 (10 g 40.48 mmol, 1.00 equiv) and CH(OMe)3 (100 mL) at room temperature. To the above mixture was added HCOOH (3 mL) and H2SO4 (1 mL) at room temperature. The resulting mixture was stirred overnight at 50 degrees C. The reaction was quenched by the addition of NaHCO3 (aq.) (300 mL) at room temperature. The aqueous layer was extracted with EtOAc (3×100 mL). The resulting mixture was concentrated under vacuum. The residue was purified by silica gel column chromatography, eluted with PE/EA (5:1) to afford Intermediate D2 (8 g 67.45%) as yellow oil.

### Synthesis of Intermediate D.

To a solution of Intermediate D2 (8 g, 26.660 mmol, 1 equiv) in 100 mL dioxane was added Pd(OAc)2 (0.60 g, 2.666 mmol, 0.1 equiv) in a pressure tank. The mixture was purged with nitrogen and then was pressurized to 10 atm with carbon monoxide/hydrogen (1:1) at 80° C. overnight. The reaction mixture was cooled to room temperature and diluted with water (600 mL). The aqueous layer was extracted with EtOAc (3×300 mL). The resulting mixture was concentrated under vacuum. The residue was purified by silica gel column chromatography, eluted with PE/EA (10:1) to afford Intermediate D (5 g, 60.21%) as a brown oil.

### Preparation of Intermediate E

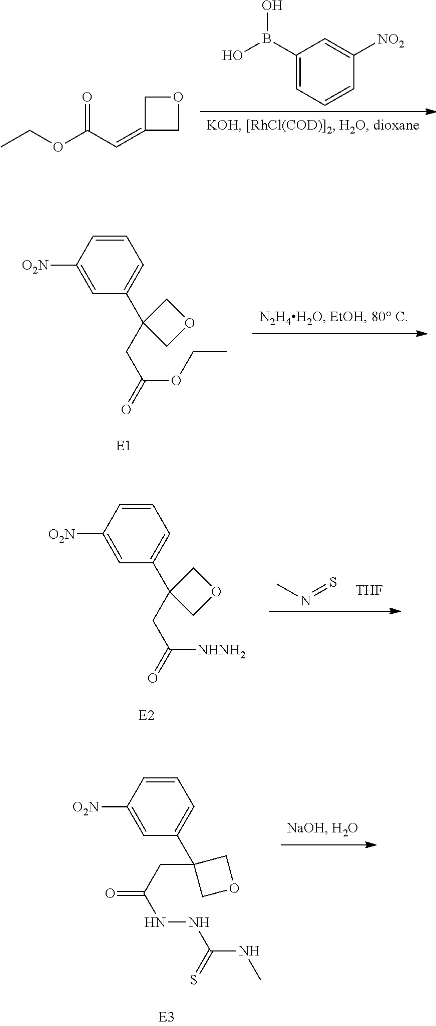

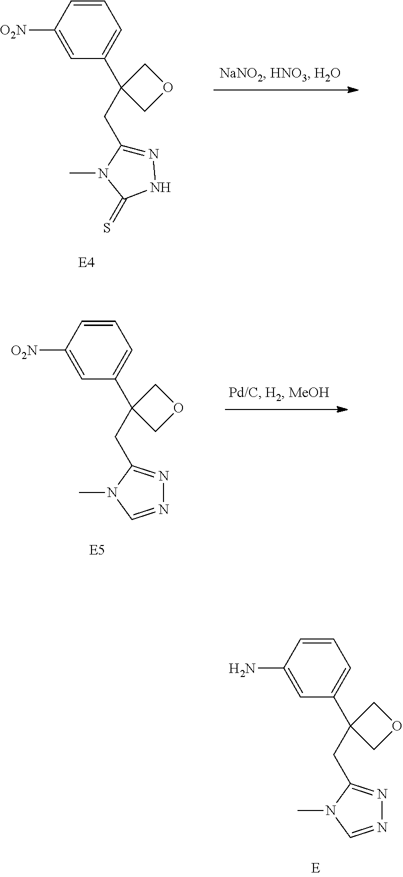 [SMILES: Cn1c(CC2(c3cccc([N+](=O)[O-])c3)COC2)n[nH]c1=S ; Cn1cnnc1CC1(c2cccc([N+](=O)[O-])c2)COC1 ; Cn1cnnc1CC1(c2cccc(N)c2)COC1]

### Synthesis of Intermediate E1.

To a stirred solution of KOH (23.68 g, 422.077 mmol, 1.2 equiv) in H₂O (285.00 mL) and dioxane (1000.00 mL) were added [Rh(COD)Cl₂ (4.00 g, 8.112 mmol, 0.02 equiv) in portions at room temperature under nitrogen atmosphere. The resulting mixture was stirred for 1 h at room temperature under nitrogen atmosphere. To the above mixture were added ethyl 2-(oxetan-3-ylidene)acetate (50.00 g, 351.731 mmol, 1.00 equiv) and 3-nitrophenylboronic acid (117.43 g, 703.462 mmol, 2 equiv) in portions at room temperature. The resulting mixture was stirred for an additional 16 h at room temperature under nitrogen atmosphere. The reaction was quenched by the addition of NH₄Cl (aq.) (3 L) at room temperature. The aqueous layer was extracted with EtOAc (3×5 L). The resulting mixture was concentrated under reduced pressure. The residue was purified by silica gel column chromatography, eluted with PE/EtOAc (10:1) to afford Intermediate E1 (41 g, 73.24%) as a yellow solid.

### Synthesis of Intermediate E2.

A mixture of Intermediate E1 (30.00 g, 113.094 mmol, 1.00 equiv) in EtOH (150 mL) and hydrazine hydrate (98%) (45.29 g, 904.756 mmol, 8 equiv) was stirred for 24 h at 80° C. The mixture was allowed to cool down to room temperature. The resulting mixture was diluted with water (750 mL). The aqueous layer was extracted with CH₂Cl₂/MeOH (10/1) (5×1 L). The resulting oil was dried with anhydrous sodium sulfate. The resulting mixture was filtered, and the filter cake was washed with MeOH (3×100 mL). The filtrate was concentrated under reduced pressure to afford Intermediate E2 (26.0 g, crude) as a yellow oil.

### Synthesis of Intermediate E3.

To a stirred solution of Intermediate E2 (26.00 g, 103.486 mmol, 1.00 equiv) in tetrahydrofuran (260.00 mL) was added methyl isothiocyanate (15.13 g, 206.972 mmol, 2.00 equiv) at room temperature. The resulting mixture was stirred for 4 h at room temperature. The resulting mixture was diluted with water (600 mL). The precipitated solids were collected by filtration and washed with water (3×50 mL) to afford Intermediate E3 (35.0 g) as a yellow solid.

### Synthesis of Intermediate E4.

To a stirred solution of Intermediate E3 (35 g, 107.905 mmol, 1.00 equiv) was added NaOH (864 mL, 863.240 mmol, 8.00 equiv, 1 M) at room temperature. The resulting mixture was stirred for 2 h at room temperature. The resulting mixture was diluted with water (1 L). The mixture was acidified to pH 5 with HCl (1 M). The aqueous layer was extracted with CH₂Cl₂/MeOH (10/1) (3×2 L). The resulting mixture was concentrated under reduced pressure to afford Intermediate E4 (24 g) as a yellow solid.

### Synthesis of Intermediate E5.

To a stirred solution of Intermediate E4 (24.00 g, 78.344 mmol, 1.00 equiv) and NaNO₂ (54.05 g, 783.443 mmol, 10.00 equiv) in H₂O (150.00 mL) and ethyl acetate (50.0 mL) was added HNO₃ (500 mL, 783.443 mmol, 10.00 equiv, 1 M) dropwise at 0° C. The resulting mixture was stirred overnight at 0° C. The reaction was quenched by the addition of NaHCO₃ (aq.) (1 L) at room temperature. The aqueous layer was extracted with CH₂Cl₂/MeOH (10/1) (3×2 L). The resulting mixture was concentrated under reduced pressure to afford Intermediate E5 (19 g) as a yellow solid.

### Synthesis of Intermediate E.

To a solution of Intermediate E5 (19.00 g) in 190 mL MeOH was added Pd/C (30%, 5.7 g) under nitrogen atmosphere in a 500 mL round-bottom flask. The mixture was hydrogenated at room temperature for 4 h under hydrogen atmosphere using a hydrogen balloon, filtered through a Celite pad and concentrated under reduced pressure. This resulted in Intermediate E (16 g) as a yellow solid.

### Preparation of Intermediate F

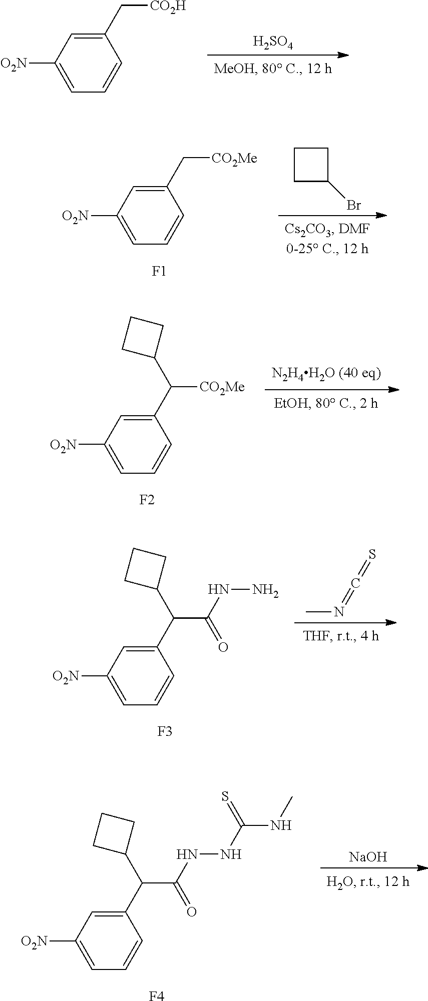

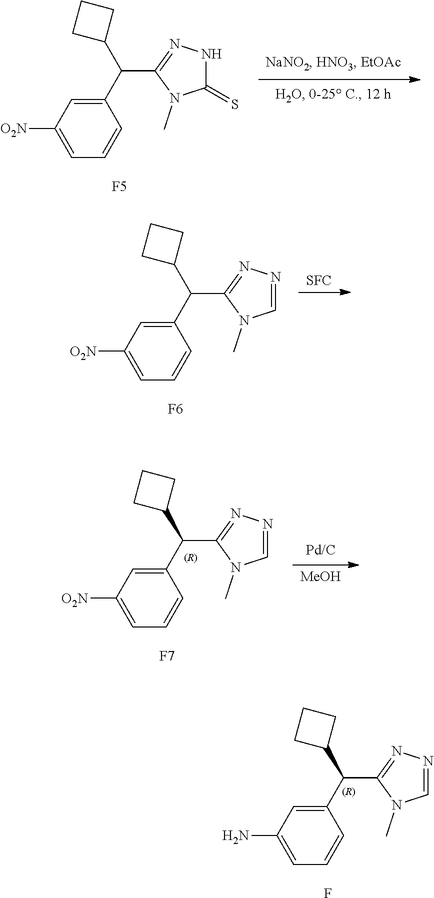

### Synthesis of Intermediate F1.

To a solution of 2-(3-nitrophenyl)acetic acid (100.0 g, 552.1 mmol, 1 equiv) in MeOH (1000.0 mL) was added H2SO4 (10.9 g, 110.4 mmol, 5.9 mL, 0.2 equiv). The mixture was stirred at 80° C. for 12 hours. The reaction was poured into water (1000.0 mL) and the resulting mixture was extracted with EtOAc (700.0 mL×2). The organic phase was washed with NaHCO₃ (300.0 ml) and brine (300.0 mL), dried over anhydrous Na2SO4, concentrated in vacuum to give a residue to afford Intermediate F1 (104.0 g, 96.53% yield) was as yellow oil.

H-NMR-Intermediate F1: (400 MHz, DMSO-d6) δ ppm 8.17-8.22 (m, 1H), 8.11-8.16 (m, 1H), 7.71-7.78 (m, 1H), 7.59-7.67 (m, 1H), 3.86-3.95 (s, 2H), 3.58-3.68 (s, 3H).

### Synthesis of Intermediate F2.

To a solution of Intermediate F1 (30.0 g, 153.7 mmol, 1.0 equiv) in DMF (300.0 mL) was added Cs2CO3 (250.4 g, 768.5 mmol, 5.0 equiv) at 0° C. The mixture was stirred at 0° C. for 3 hours. Then the mixture was added bromocyclobutane (62.3 g, 461.1 mmol, 43.5 mL, 3.0 equiv) at 25° C. The mixture was stirred at 25° C. for 9 hours. The reaction was poured into water (500.0 mL) and the resulting mixture was extracted with EtOAc (300.0 mL×2). The organic phase was washed with brine (300.0 mL), dried over anhydrous Na2SO4, concentrated in vacuum to give a residue. The residue was purified by column chromatography (SiO2, Petroleum ether/Ethyl acetate=0/1 to 3/1) to afford Intermediate F2 (35.0 g, 91.35% yield) as colorless oil.

### Synthesis of Intermediate F3.

To a solution of Intermediate F2 (76.0 g, 304.9 mmol, 1.0 equiv) in EtOH (800.0 mL) was added hydrazine; hydrate (622.9 g, 12.2 mol, 604.8 mL, 98% purity, 40.0 equiv). The mixture was stirred at 80° C. for 2 hours. The reaction mixture was concentrated under reduced pressure to remove solvent. The mixture was added H2O (800.0 mL) and EtOAc (800.0 mL). The organic phase was washed with NaHCO₃ (300.0 ml) and brine (300.0 mL), dried over anhydrous Na2SO4, concentrated in vacuum to give a residue. Intermediate F3 (38.0 g, 40.00% yield) was obtained as a yellow solid.

### Synthesis of Intermediate F4.

To a solution of Intermediate F3 (35.0 g, 140.4 mmol, 1.0 equiv) in THF (350.0 mL) was added methylimino(thioxo)methane (20.5 g, 280.8 mmol, 19.2 mL, 2.0 equiv). The mixture was stirred at 25° C. for 4 hours. The reaction was poured to water (600.0 mL) to give white solid. The solid was filtered and concentrated to afford Intermediate F4 (39.0 g, 86.16% yield) as a white solid.

### Synthesis of Intermediate F5.

To a solution of NaOH (36.7 g, 918.1 mmol, 8.0 equiv) in H2O (500.0 mL) was added Intermediate F4 (37.0 g, 114.8 mmol, 1.0 equiv). The mixture was stirred at 25° C. for 2 hours. The reaction mixture was acidified by 1M HCl to pH=3˜4 to form solid. The solid was filtered and the filter cake was concentrated under reduced pressure to afford Intermediate F5 (39.0 g, crude) as a white solid.

### Synthesis of Intermediate F6.

HNO3 (112.6 g, 1.2 mol, 80.5 mL, 68% purity, 10.0 equiv) was added to H2O (599.0 mL) to afford the diluted HNO3 solution (2M, 690.0 mL). To a solution of Intermediate F5 (37.0 g, 121.6 mmol, 1.0 equiv) and NaNO2 (83.8 g, 1.2 mol, 10.0 equiv) in H2O (420.0 mL) and EtOAc (42.0 mL) was added diluted HNO3 solution (1 M, 6.2 mL) dropwise at 0° C. Then the mixture was stirred at 25° C. for 12 hours. The reaction mixture was neutralized by a.q. NaHCO3 to pH=7-8, and the resulting mixture was extracted with EtOAc (800.0 mL×2). The organic phase was washed with brine (500.0 mL), dried over anhydrous Na2SO4, concentrated in vacuum to give Intermediate F6 (34.0 g, 98.19% yield) as a white solid.

H-NMR-Intermediate F6: (400 MHz, DMSO-d6) 8.36 (s, 1H), 8.14-8.20 (m, 1H), 8.06-8.13 (m, 1H), 7.76 (br d, J=7.6 Hz, 1H), 7.58-7.67 (m, 1H), 4.42-4.54 (m, 1H), 3.45 (s, 3H), 3.05-3.21 (m, 1H), 1.99-2.12 (m, 1H), 1.65-1.88 (m, 5H).

### Synthesis of Intermediate F7.

The racemate of Intermediate F6 was purified by SFC (column: DAICEL CHIRALPAK IC (250 mm*50 mm, 10 um); mobile phase: [0.1% NH3H2O MEOH]; B %: 55%-55%, 9.5 min) to afford Intermediate F7 (29 g, 48.00% yield) as a yellow solid.

H-NMR-Intermediate F7: (400 MHz, DMSO-d6) δ ppm 8.32-8.42 (m, 1H), 8.13-8.19 (m, 1H), 8.07-8.13 (m, 1H), 7.72-7.80 (m, 1H), 7.58-7.68 (m, 1H), 4.43-4.52 (m, 1H), 3.39-3.51 (m, 3H), 3.07-3.16 (m, 1H), 1.99-2.11 (m, 1H), 1.67-1.86 (m, 5H).

### Synthesis of Intermediate F.

A mixture of Intermediate F7 (2.0 g, 7.3 mmol, 1.0 equiv), Pd/C (1.0 g, 10% purity) in MeOH (15.0 mL) was degassed and purged with H2 for 3 times, and then the mixture was stirred at 30° C. for 12 hours under H2 atmosphere (50 psi). The reaction mixture was filtered and the filtrate was concentrated to afford Intermediate F (1.7 g, 95.52% yield) as a black solid.

H-NMR-Intermediate F: (400 MHz, DMSO-d6) δ ppm 8.22-8.33 (m, 1H), 6.86-6.96 (m, 1H), 6.28-6.43 (m, 3H), 4.90-5.10 (m, 2H), 3.85-3.95 (m, 1H), 3.31-3.35 (m, 3H), 3.01-3.16 (m, 1H), 1.95-2.16 (m, 1H), 1.71-1.80 (m, 4H), 1.58-1.68 (m, 1H).

### Preparation of Intermediate G

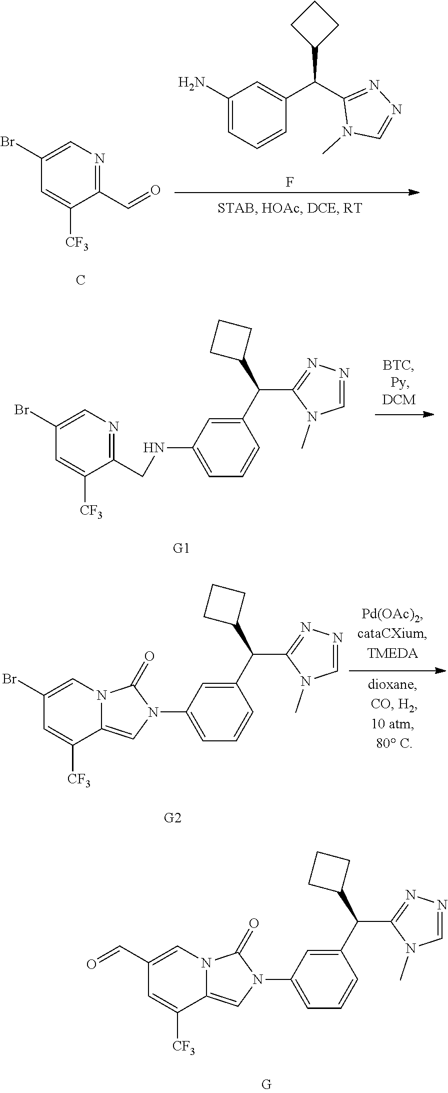

### Synthesis of Intermediate G1.

To a stirred solution of Intermediate C (10.38 g, 40.854 mmol, 1.1 equiv) and Intermediate F (9 g, 37.140 mmol, 1.00 equiv) in DCE (120 mL) were added STAB (15.74 g, 74.280 mmol, 2 equiv) and HOAc (2.23 g, 37.140 mmol, 1 equiv) at room temperature. The resulting mixture was stirred for overnight at room temperature. The reaction was quenched by the addition of Sat. NH4Cl(aq) (200 mL) at room temperature. The resulting mixture was extracted with CH2Cl2 (3×350 mL). The resulting mixture was concentrated under reduced pressure. The residue was purified by silica gel column chromatography, eluted with CH2Cl2/MeOH (50:1) to afford Intermediate G1 (14 g, 74.55%) as a light yellow oil.

### Synthesis of Intermediate G2.

To a stirred solution of Intermediate G1 (14 g, 29.146 mmol, 1 equiv) and Pyridine (13.83 g, 174.876 mmol, 6 equiv) in DCM (150 mL) were added Triphosgene (3.03 g, 10.201 mmol, 0.35 equiv) at 0° C. The resulting mixture was stirred for 20 min at room temperature. The reaction was quenched by the addition of Sat. NaHCO3 (aq) (200 mL) at room temperature. The resulting mixture was extracted with CH2Cl2 (3×300 mL). The resulting mixture was concentrated under vacuum. The residue was purified by trituration with MTBE (20 mL) to afford Intermediate G2 (12 g, 77.25%) as a yellow solid.

### Synthesis of Intermediate G.

To a solution of Intermediate G2 (14 g, 27.650 mmol, 1 equiv), TMEDA (6.43 g, 55.300 mmol, 2 equiv) in dioxane (400.00 mL) was added bis(adamantan-1-yl)(butyl)phosphane (1.98 g, 5.530 mmol, 0.2 equiv) and Pd(OAc)2 (0.62 g, 2.765 mmol, 0.1 equiv) in an autoclave. After flushing the autoclave three times with CO/H2 (1:1), the mixture was pressurized to 10 atm with CO/H2 (1:1) at 80° C. for overnight. The resulting mixture was concentrated under vacuum. The residue was purified by silica gel column chromatography eluted with CH2Cl2/MeOH (20:1) to afford Intermediate G (12 g, 90.53%) as a yellow solid.

### Preparation of Intermediate H

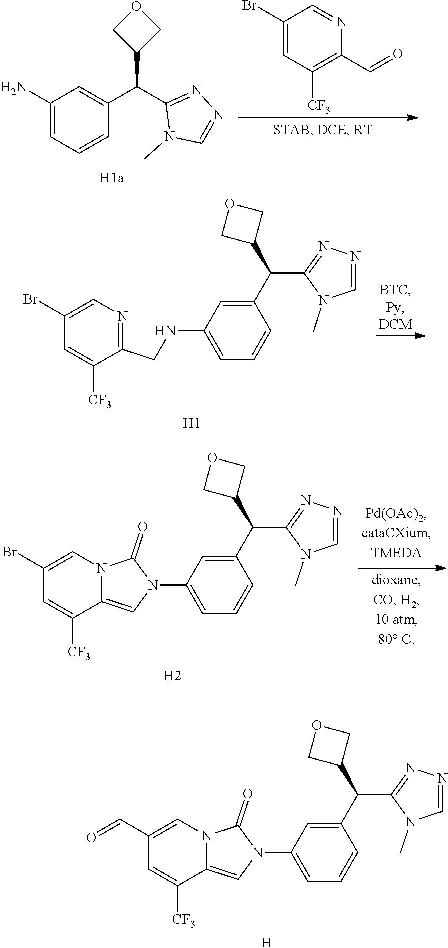

### Synthesis of Intermediate H1.

To a stirred solution of H1a (3.4 g, 13.917 mmol, 1 equiv) and 5-bromo-3-(trifluoromethyl)picolinaldehyde (4.24 g, 16.700 mmol, 1.2 equiv) in DCE (100 mL) was added STAB (5.90 g, 27.834 mmol, 2 equiv) at room temperature. The resulting mixture was stirred overnight at room temperature. The reaction was quenched with saturated NaHCO₃ (aq.) (300 mL) at room temperature. The aqueous layer was extracted with DCM/MeOH=10:1 (3×200 mL). The resulting mixture was concentrated under vacuum. The residue was purified by silica gel column chromatography, eluted with DCM/MeOH (20:1) to afford Intermediate H1 (4 g, 57.80%) as a Brown yellow solid.

### Synthesis of Intermediate H2.

To a stirred solution of Intermediate H1 (4 g, 8.294 mmol, 1 equiv) and Pyridine (6.56 g, 82.933 mmol, 10.00 equiv) in DCM (100 mL) was added Triphosgene (0.98 g, 3.318 mmol, 0.40 equiv) at 0° C. The resulting mixture was stirred for 10 min at room temperature. The reaction was quenched with saturated NaHCO₃ (aq.) (200 mL) at room temperature. The aqueous layer was extracted with DCM (2×200 mL). The resulting mixture was concentrated under vacuum. The residue was purified by trituration with MTBE (100 mL). This resulted in Intermediate H2 (3.5 g, 75.55%) as a reddish brown solid.

### Synthesis of Intermediate H.

To a solution of Intermediate H2 (3.4 g, 6.689 mmol, 1 equiv) in dioxane (100 mL) was added Pd(OAc)₂ (0.15 g, 0.669 mmol, 0.1 equiv) and bis(adamantan-1-yl)(butyl)phosphane (0.48 g, 1.338 mmol, 0.2 equiv) in a pressure tank. The mixture was purged with nitrogen for 3 min and then was pressurized to 15 atm with CO/H₂=1:1 at 80° C. for overnight. The reaction mixture was cooled to room temperature. The resulting mixture was concentrated under vacuum. The residue was purified by silica gel column chromatography, eluted with DCM/MeOH (20:1) to afford Intermediate H (1.1 g, 32.36%) as a reddish brown solid.

### Preparation of Intermediate I

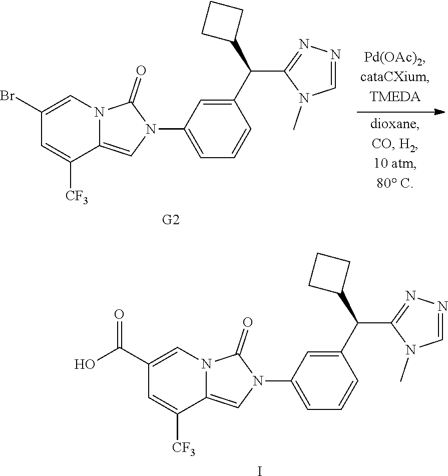

### Synthesis of Intermediate I.

Into a 1 L pressure tank reactor were added Intermediate G2 (20 g, 19.750 mmol, 1 equiv), dioxane (600 mL), TMEDA (6.89 g, 59.250 mmol, 3 equiv), bis(adamantan-1-yl)(butyl)phosphane (1.42 g, 3.950 mmol, 0.2 equiv) and Pd(OAc)2 (0.44 g, 1.975 mmol, 0.1 equiv) at room temperature. After flushing the autoclave three times with CO/H2 (1:1), the mixture was pressurized to 10 atm with CO/H2 (1:1) at 80° C. for overnight. The mixture was allowed to cool down to room temperature. The resulting mixture was concentrated under vacuum. The residue was purified by silica gel column chromatography, eluted with CH2Cl2/MeOH (10:1) to afford Intermediate I (3 g, 16.11%) as a yellow solid.

### Preparation of Intermediate J

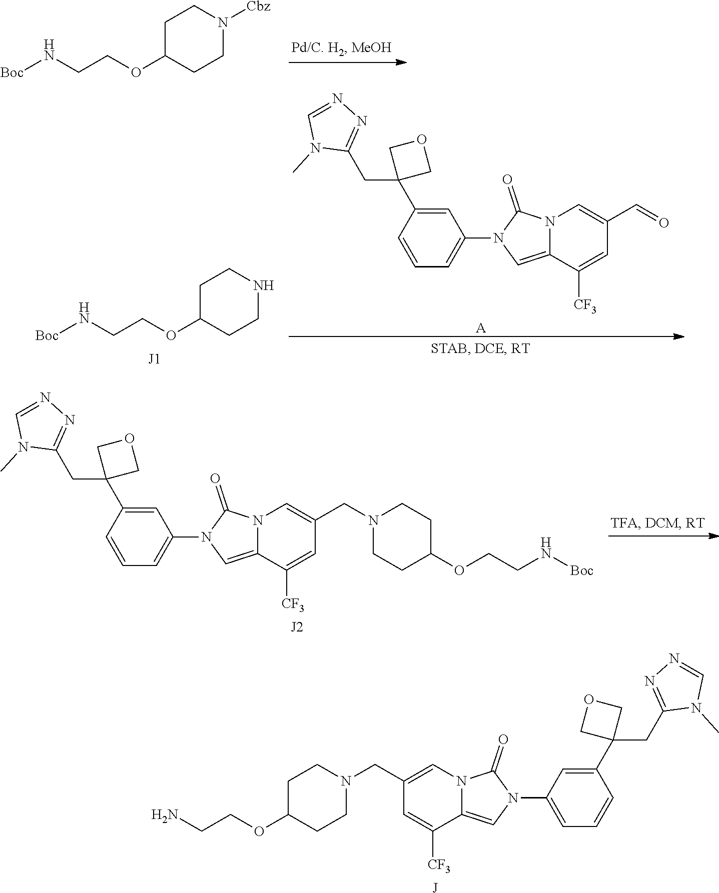

### Synthesis of Intermediate J1.

To a solution of benzyl 4-(2-((tert-butoxycarbonyl)amino)ethoxy)piperidine-1-carboxylate (12 g, 31.70 mmol, 1.0 equiv) in 360 mL MeOH was added Pd/C (10%, 2 g) under nitrogen atmosphere in a 500 mL round-bottom flask. The mixture was hydrogenated at room temperature for overnight under hydrogen atmosphere using a hydrogen balloon, filtered through a Celite pad and concentrated under reduced pressure. This resulted in Intermediate J1 (7 g, 90%) as a colorless oil.

### Synthesis of Intermediate J2.

To a stirred solution of Intermediate J1 (1 g, 4.09 mmol, 1.0 equiv) and Intermediate A (1.8 g, 4.09 mmol, 1.0 equiv) in DCE (10 mL) was added STAB (1.7 g, 8.18 mmol, 2.0 equiv) at room temperature. The resulting mixture was stirred for overnight at room temperature. The reaction was quenched by the addition of water (30 mL) at room temperature. The resulting mixture was extracted with CH₂Cl₂ (3×30 mL). The combined organic layers were washed with water (3×30 mL), dried over anhydrous Na₂SO₄. After filtration, the filtrate was concentrated under reduced pressure. The residue was purified by Prep-TLC (CH₂Cl₂/MeOH=10:1) to afford Intermediate J2 (900 mg, 32%) as a yellow solid.

### Synthesis of Intermediate J.

Intermediate J was prepared from Boc deprotection of Intermediate J2, which was performed in a manner similar to the procedure described in Example 2.

### Preparation of Intermediate K

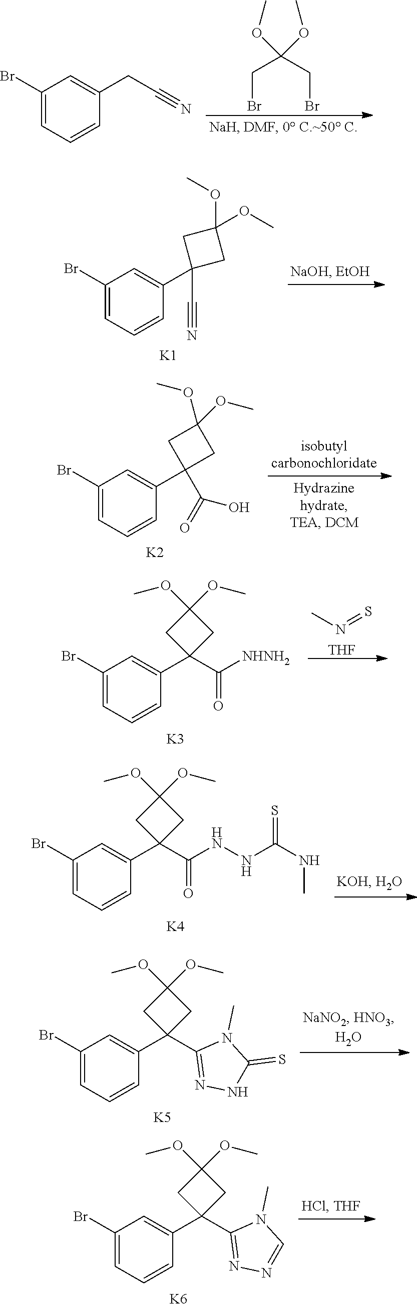

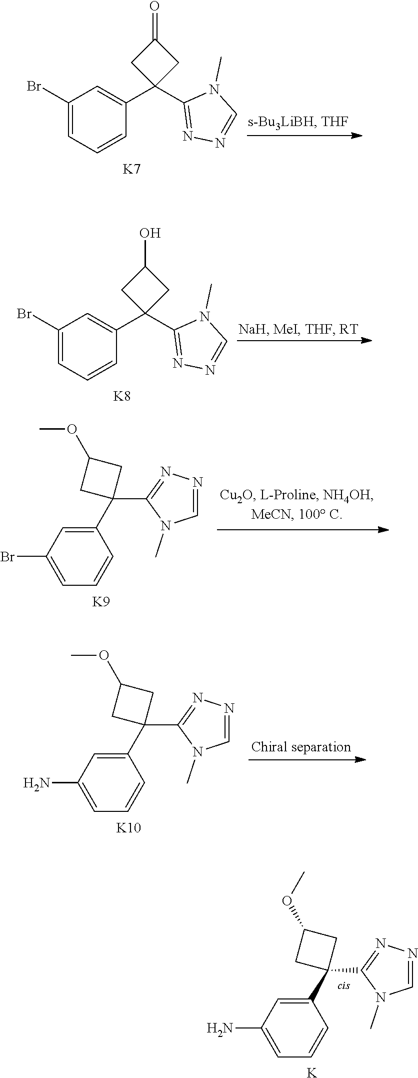

### Synthesis of Intermediate K1.

To a stirred solution of NaH (24.48 g, 1020.164 mmol, 2 equiv) in DMF (1000 mL) was added 2-(3-bromophenyl) acetonitrile (100 g, 510.082 mmol, 1 equiv) dropwise at 0° C. under nitrogen atmosphere, followed by 1,3-dibromo-2,2-dimethoxypropane (133.61 g, 510.082 mmol, 1 equiv) at 60° C. under nitrogen atmosphere. The resulting mixture was stirred overnight at 60° C. under nitrogen atmosphere. The reaction was quenched with NH4Cl (aq.) (2 L) at room temperature. The aqueous layer was extracted with EtOAc (2×1000 mL). The resulting mixture was concentrated under reduced pressure. The residue was purified by silica gel column chromatography, eluted with PE/EA (10:1) to afford Intermediate K1 (60 g, 39.72%) as off-white solid.

### Synthesis of Intermediate K2.

To a stirred solution of Intermediate K1 (60 g, 202.590 mmol, 1 equiv) in EtOH (600 mL) was added NaOH (28.36 g, 709.065 mmol, 3.5 equiv) in H2O (600 mL) at room temperature. The resulting mixture was stirred overnight at 80° C. The EtOH was concentrated under vacuum. The mixture was acidified to pH 2 with HCl (1M). The aqueous layer was extracted with EtOAc (4×300 mL). The combined organic layers were dried over anhydrous Na2SO4. After filtration, the filtrate was concentrated under reduced pressure. This resulted in Intermediate K2 (43 g, 67.35%) as off-white solid.

### Synthesis of Intermediate K3.

To a stirred solution of Intermediate K2 (43 g, 136.437 mmol, 1 equiv) and TEA (27.61 g, 272.874 mmol, 2 equiv) in DCM (1500 mL) was added isobutyl carbonochloridate (27.95 g, 204.656 mmol, 1.5 equiv) dropwise at 0° C. under nitrogen atmosphere. The resulting mixture was stirred for 30 min at 0° C. To the above mixture was added hydrazine hydrate (27.32 g, 545.748 mmol, 4 equiv) dropwise at −30° C. The resulting mixture was stirred for additional 40 min at room temperature. The reaction was quenched with water (2 L) at room temperature. The aqueous layer was extracted with CH2Cl2 (3×800 mL). The combined organic layers were dried over anhydrous Na2SO4. After filtration, the filtrate was concentrated under reduced pressure. This resulted in Intermediate K3 (50 g, 83.49%) as off-white solid. The crude product was used in the next step directly without further purification.

### Synthesis of Intermediate K4.

To a stirred solution of Intermediate K3 (50 g, 113.915 mmol, 1 equiv) in tetrahydrofuran (500 mL) was added methyl isothiocyanate (24.98 g, 341.745 mmol, 3 equiv) at room temperature. The resulting mixture was stirred for overnight at room temperature. The resulting mixture was diluted with water (400 mL). The resulting mixture was concentrated under vacuum. The precipitated solids were collected by filtration and washed with water (200 mL). This resulted in Intermediate K4 (65 g, 99.28%) as off-white solid. The crude product was used in the next step directly without further purification.

### Synthesis of Intermediate K5.

To a stirred solution of KOH (72.52 g, 1292.536 mmol, 8 equiv) in H2O (1.2 L) was added Intermediate K4 (65 g, 161.567 mmol, 1 equiv) at room temperature. The resulting mixture was stirred overnight at room temperature. The residue was neutralized to pH 7 with HCl (aq.) (1M). The precipitated solids were collected by filtration and washed with water (200 mL). This resulted in Intermediate K5 (45 g, 72.48%) as off-white solid.

### Synthesis of Intermediate K6.

To a stirred mixture of Intermediate K5 (45 g, 117.099 mmol, 1 equiv) and NaNO2 (80.79 g, 1170.990 mmol, 10.00 equiv) in EA (250 mL)/H2O (250 ml) was added HNO3 (1170 mL, 1170.990 mmol, 10.00 equiv, 1M) dropwise at room temperature. The resulting mixture was stirred overnight at room temperature. The mixture was neutralized to pH 7 with saturated Na2CO3 (aq.). The aqueous layer was extracted with EtOAc (5×500 L). The resulting mixture was concentrated under vacuum. This resulted in Intermediate K6 (35 g, 65.34%) as off-white solid.

### Synthesis of Intermediate K7.

To a stirred solution of Intermediate K6 (35 g, 78.747 mmol, 1 equiv, 77%) in THF (200 mL) was added HCl (150 mL, 4M) at room temperature. The resulting mixture was stirred overnight at 80° C. The mixture was neutralized to pH 8 with NaHCO₃ (aq.). The aqueous layer was extracted with EtOAc (3×100 mL). The resulting mixture was concentrated under vacuum. The residue was purified by reverse flash chromatography with the following conditions: column, C18 silica gel; mobile phase, MeCN in water, 0% to 40% gradient in 20 min; detector, UV 220 nm. This resulted in Intermediate K7 (18 g, 74.66%) as a white solid.

### Synthesis of Intermediate K8.

To a stirred solution of Intermediate K7 (34.5 g, 112.685 mmol, 1 equiv) in THF (400 mL) was added lithium triisobutylhydroborate (42.84 g, 225.370 mmol, 2 equiv) dropwise at 0° C. The resulting mixture was stirred for 4 h at room temperature. The reaction was quenched with NH4Cl (aq.) (500 mL) at room temperature. The aqueous layer was extracted with EtOAc (5×400 mL). The resulting mixture was concentrated under vacuum. The residue was purified by reverse flash chromatography with the following conditions: column, C18 silica gel; mobile phase, MeCN in Water (0.1% FA), 10% to 60% gradient in 30 min; detector, UV 220 nm. This resulted in Intermediate K8 (17 g, 48.95%) as a white solid.

### Synthesis of Intermediate K9.

To a stirred mixture of Intermediate K8 (17 g, 55.163 mmol, 1 equiv) in THF (200 mL) was added NaH (2.65 g, 110.326 mmol, 2 equiv) at 0° C. under nitrogen atmosphere. The resulting mixture was stirred for 1 h at 0° C. under nitrogen atmosphere. To the above mixture was added MeI (9.40 g, 66.196 mmol, 1.2 equiv) at 0° C. The resulting mixture was stirred overnight at room temperature. The reaction was quenched with NH4Cl (aq.) (500 mL) at room temperature. The aqueous layer was extracted with EtOAc (4×300 mL). The resulting mixture was concentrated under vacuum. The residue was purified by reverse flash chromatography with the following conditions: column, C18 silica gel; mobile phase, MeCN in Water (0.1% FA), 10% to 60% gradient in 30 min; detector, UV 220 nm. This resulted in Intermediate K9 as a white solid.

### Synthesis of Intermediate K10.

To a stirred mixture of Intermediate K9 (11 g, 34.140 mmol, 1 equiv) and Cu2O (4.89 g, 34.140 mmol, 1 equiv) in NH4OH (500 mL) and MeCN (500 mL) was added L-Proline (0.83 g, 3.414 mmol, 0.1 equiv) at room temperature. The resulting mixture was stirred overnight at 100° C. The mixture was allowed to cool down to room temperature. The resulting mixture was filtered; the filter cake was washed with MeOH (3×100 mL). The resulting mixture was concentrated under vacuum. The residue was purified by reverse flash chromatography with the following conditions: column, C18 silica gel; mobile phase, MeCN in Water (0.1% FA), 10% to 50% gradient in 30 min; detector, UV 254 nm. This resulted in Intermediate K10 (6.2 g, 70.30%) as off-white solid.

### Synthesis of Intermediate K.

The Intermediate K10 (6.2 g) was purified by Prep-SFC with the following conditions (Column: Lux 5 um Cellulose-3, 5*25 cm, 5 μm; Mobile Phase A: CO2, Mobile Phase B: MeOH (0.1% 2M NH3-MEOH); Flow rate: 150 mL/min; Gradient: isocratic 20% B; Column Temperature (° C.): 35; Back Pressure (bar): 100; Wave Length: 220 nm; RT1 (min): 4.78; RT2 (min): 5.94; the first peak was product) to afford Intermediate K (2.7 g) as an off-white solid.

### Preparation of Intermediate L

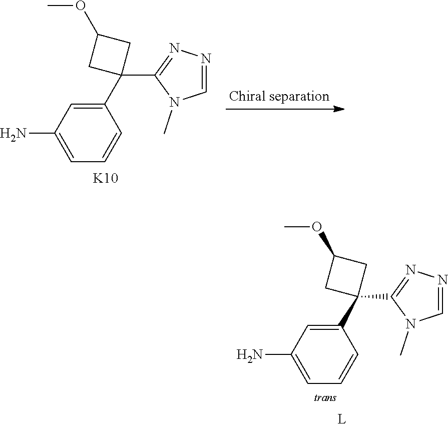

### Synthesis of Intermediate L.

Intermediate K10 (10 g) was purified by Prep-SFC with the following conditions (Column: Lux 5 um Cellulose-3, 3*25 cm, 5 μm; Mobile Phase A: CO₂, Mobile Phase B: MEOH (0.1% 2M NH₃-MEOH); Flow rate: 70 mL/min; Gradient: isocratic 25% B; Column Temperature (° C.): 35; Back Pressure (bar): 100; Wave Length: 220 nm; RT1 (min): 4.5; RT2 (min): 6.8; Sample Solvent: MeOH—Preparative; Injection Volume: 1.9 mL; Number Of Runs: 16, the second peak is product) to give Intermediate L (4.8 g, 48%) as a white solid.

### Preparation of Intermediate M

Intermediate M can be prepared using methods known in the art, for example, those methods described in Example Z on page 342 of WO2019148005.

### Example 1. Compound 1

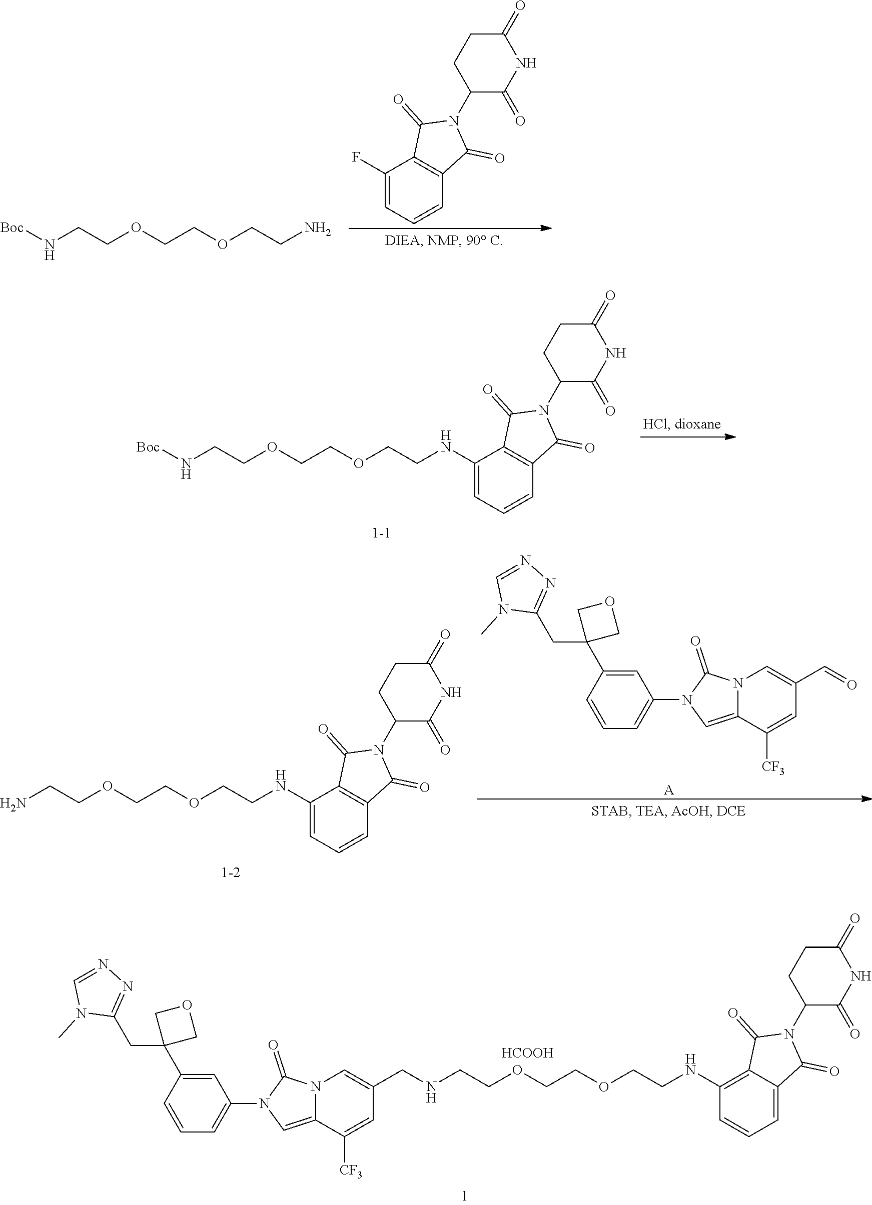

### Synthesis of Compound 1-1

To a stirred solution of tert-butyl N-{2-[2-(2-aminoethoxy) ethoxy] ethyl} carbamate (2 g, 8.054 mmol, 1 equiv) and 2-(2,6-dioxopiperidin-3-yl)-4-fluoroisoindole-1,3-dione (2.67 g, 9.665 mmol, 1.2 equiv) in NMP (20 mL) was added DIEA (3.12 g, 24.162 mmol, 3 equiv) at room temperature under nitrogen atmosphere. The resulting mixture was stirred for overnight at 90° C. under nitrogen atmosphere. The reaction was quenched by the addition of water (30 mL) at room temperature. The resulting mixture was extracted with EtOAc (3×50 mL). The resulting mixture was concentrated under reduced pressure. The residue was purified by silica gel column chromatography, eluted with CH₂Cl₂/MeOH (50:1) to afford Compound 1-1 (1.2 g, 28.05%) as a light yellow oil.

### Synthesis of Compound 1-2

To a stirred solution of Compound 1-1 (1.2 g, 2.378 mmol, 1 equiv) in HCl(g) in 1,4-dioxane (15 mL) at room temperature. The resulting mixture was stirred for 2 h at room temperature. The resulting mixture was concentrated under vacuum. This resulted in Compound 1-2 (800 mg, 74.85%) as a light yellow oil.

### Synthesis of Compound 1

To a stirred solution of Compound 1-2 (200 mg, 0.495 mmol, 1.00 equiv) and Intermediate A (226.21 mg, 0.495 mmol, 1 equiv) in DCE (3 mL) were added STAB (209.62 mg, 0.990 mmol, 2 equiv), TEA (100.09 mg, 0.990 mmol, 2 equiv) and AcOH (29.70 mg, 0.495 mmol, 1 equiv) at room temperature. The resulting mixture was stirred for overnight at room temperature. The reaction was quenched by the addition of water (3 mL) at room temperature. The resulting mixture was extracted with EtOAc (3×5 mL). The resulting mixture was concentrated under reduced pressure. The crude product (25 mg) was purified by Prep-HPLC with the following conditions (Column: XSelect CSH Prep C18 OBD Column, 19*250 mm, 5 μm; Mobile Phase A: Water (0.1% FA), Mobile Phase B: ACN; Flow rate: 30 mL/min; Gradient: 15% B to 26% B in 10 min, 26% B; Wave Length: 254; 220 nm; RT1 (min): 9.90) to afford Compound 1 (12.4 mg, 2.74%) as a yellow solid.

LC-MS-Compound 1: (ES, m/z): [M−HCOOH+H]⁺: 846

H-NMR-Compound 1: (400 MHz, DMSO-d6, ppm): δ 2.05-2.15 (m, 1H), δ 2.64-2.78 (m, 2H), δ 2.83-2.94 (m, 1H), δ 2.95-3.04 (m, 3H), δ 3.06-3.10 (m, 2H), δ 3.51-3.58 (m, 2H), δ 3.62-3.97 (m, 13H), δ 5.05-5.14 (m, 5H), δ 6.87-6.88 (d, 1H), δ 6.94-6.97 (m, 2H), δ 7.05-7.09 (m, 2H), δ 7.31-7.32 (d, 1H), δ 7.34-7.39 (m, 1H), δ 7.52-7.56 (m, 1H), δ 7.62-7.64 (d, 1H), δ 7.82 (s, 1H), δ 8.52 (s, 1H).

### Example 2. Compound 2

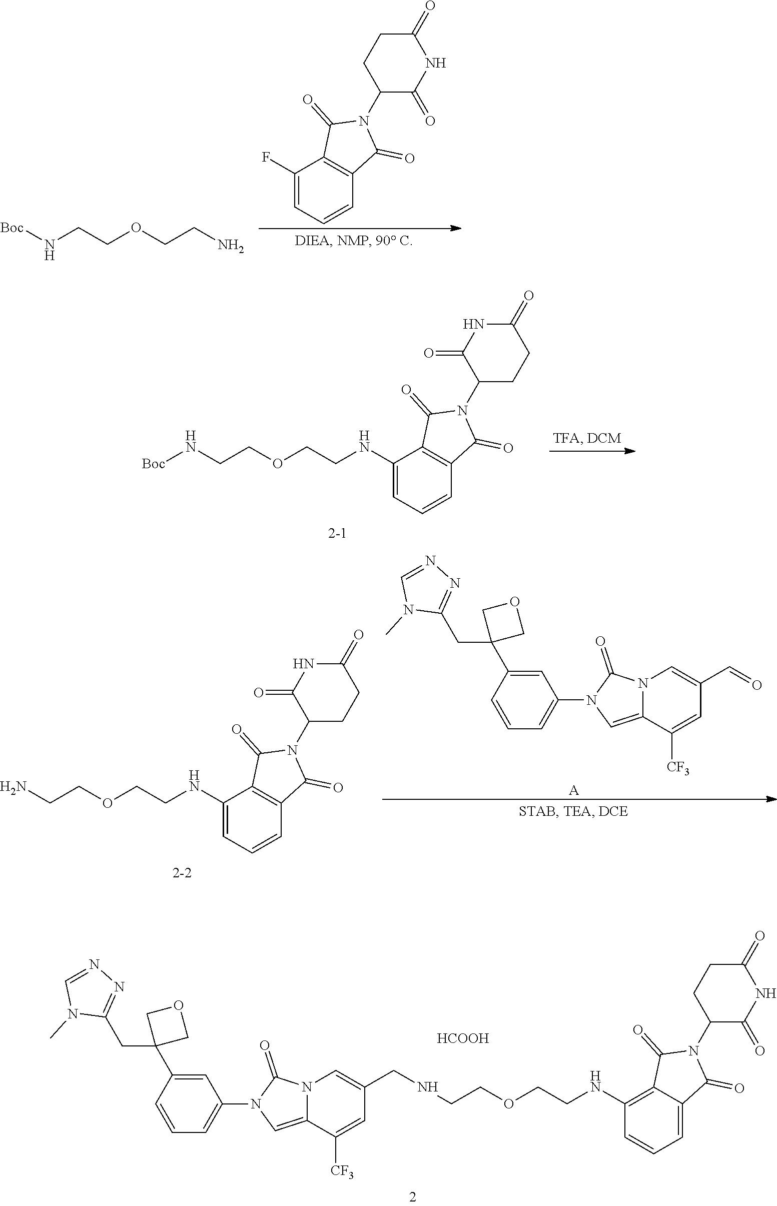

### Synthesis of Compound-2-1

To a stirred solution of tert-butyl N-[2-(2-aminoethoxy) ethyl] carbamate (1 g, 4.895 mmol, 1 equiv) in NMP (10 mL) were added 2-(2,6-dioxopiperidin-3-yl)-4-fluoroisoindole-1,3-dione (1.62 g, 5.874 mmol, 1.2 equiv) and DIEA (1.27 g, 9.790 mmol, 2 equiv) at room temperature under nitrogen atmosphere. The resulting mixture was stirred for 6 h at 90° C. under nitrogen atmosphere. The reaction was quenched by the addition of water (20 mL) at room temperature. The resulting mixture was extracted with EtOAc (3×40 mL). The resulting mixture was concentrated under reduced pressure. The residue was purified by silica gel column chromatography, eluted with PE/EA (10:1) to afford Compound 2-1 (550 mg, 23.42%) as a light yellow solid.

### Synthesis of Compound 2-2

To a stirred solution of Compound 2-1 (300 mg, 0.651 mmol, 1 equiv) in DCM (2 mL) were added TFA (2 mL) at room temperature under air atmosphere. The resulting mixture was stirred for 2 h at room temperature under air atmosphere. The resulting mixture was concentrated under reduced pressure. This resulted in Compound 2-2 (280 mg, 109.72%) as a brown yellow oil.

### Synthesis of Compound 2

To a stirred solution of Compound 2-2 (100 mg, 0.277 mmol, 1.00 equiv) and Intermediate A (139.62 mg, 0.305 mmol, 1.1 equiv) in DCE (2.00 mL) were added STAB (117.62 mg, 0.554 mmol, 2 equiv) and AcOH (16.66 mg, 0.277 mmol, 1 equiv) dropwise at room temperature under nitrogen atmosphere. The resulting mixture was stirred for overnight at room temperature under nitrogen atmosphere. The reaction was quenched by the addition of water (10 mL) at room temperature. The resulting mixture was extracted with EtOAc (3×30 mL). The resulting mixture was concentrated under reduced pressure. The crude product (34.00 mg) was purified by Prep-HPLC with the following conditions (Column: Sunfire prep C18 column, 30*150 mm, 5 μm; Mobile Phase A: Water (0.1% FA), Mobile Phase B: ACN; Flow rate: 60 mL/min; Gradient: 10% B to 25% B in 7 min, 25% B to 25% B in 9 min, 25% B; Wave Length: 220 nm; RT1 (min): 7.85) to afford Compound 2 (17.2 mg, 7.69%) as a yellow solid. LC-MS-Compound 2: (ES, m/z): [M−HCOOH+H]⁺: 802

H-NMR-Compound 2: (400 MHz, DMSO-d6, ppm): δ 2.01-2.12 (m, 1H), δ 2.62-2.71 (m, 2H), δ 2.80-2.88 (m, 1H), δ 2.96-3.02 (m, 5H), δ 3.55-3.56 (m, 2H), δ 3.64-3.82 (m, 8H), δ 5.02-5.13 (m, 5H), δ 5.91-5.93 (d, 1H), δ 6.01-6.03 (d, 1H), δ 7.12-7.16 (m, 3H), δ 7.30 (s, 1H), δ 7.51-7.52 (d, 1H), δ 7.56-7.58 (d, 1H), δ 7.62-7.65 (d, 1H), δ 7.72-7.74 (d, 1H), δ 8.52 (s, 1H).

### Example 3. Compound 3

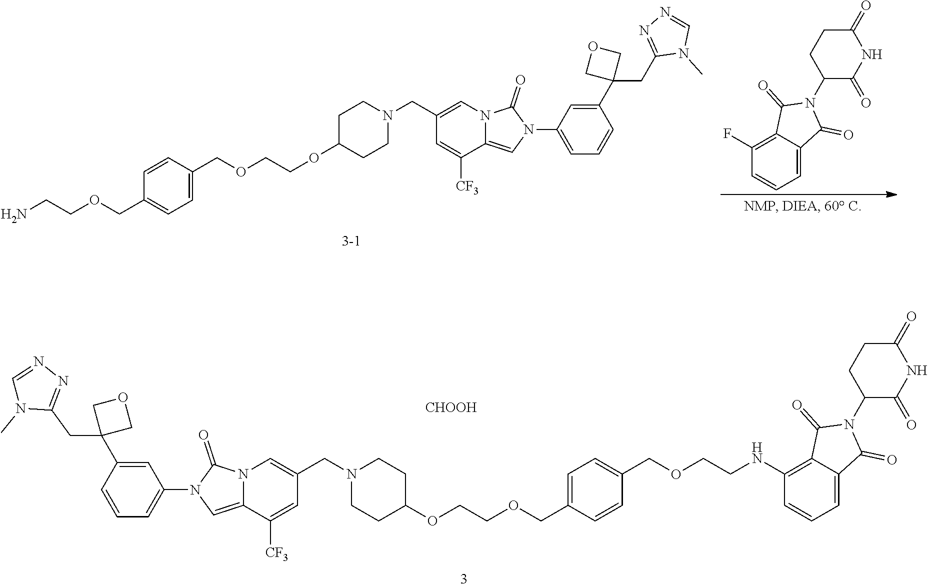

### Synthesis of Compound 3

To a stirred solution of 3-1 (prepared in a similar manner as 21-1) (100 mg, 0.133 mmol, 1 equiv) and 2-(2,6-dioxopiperidin-3-yl)-4-fluoroisoindole-1,3-dione (110.51 mg, 0.399 mmol, 3 equiv) in NMP (1 mL) was added DIEA (51.71 mg, 0.399 mmol, 3 equiv). The resulting mixture was stirred for overnight at 60° C. The reaction solution was purified by Prep-HPLC with the following conditions (Column: XSelect CSH Prep C18 OBD Column, 19*250 mm, 5 μm; Mobile Phase A: Water (0.1% FA), Mobile Phase B: ACN; Flow rate: 30 mL/min; Gradient: 28% B to 38% B in 7 min, 38% B; Wave Length: 254; 220 nm; RT1 (min): 6.05; Number Of Runs: 0) to afford Compound 3 (15.6 mg, 11.12%) as a yellow solid.

LCMS-Compound 3: (ES, m/z): [M−HCOOH-+H]⁺ 1006

NMR-Compound 3: (400 MHz, CD₃OD, δ ppm): δ1.63-1.71 (m, 2H), 1.82-1.96 (m, 2H), 2.07-2.13 (m, 1H), 2.74-2.88 (m, 5H), 2.96 (s, 3H), 3.42 (s, 2H), 3.50-3.53 (m, 3H), 3.62-3.73 (m, 8H), 4.55-4.58 (d, 4H), 5.06-5.10) (m, 5H), 6.90-6.92 (d, 1H), 7.01-7.03 (m, 2H), 7.08-7.10 (d, 2H), 7.32-7.38 (m, 5H), 7.44-7.51 (m, 2H), 7.61-7.63 (m, 1H), 7.68 (s, 1H), 8.20 (s, 1H).

### Example 4. Compound 4

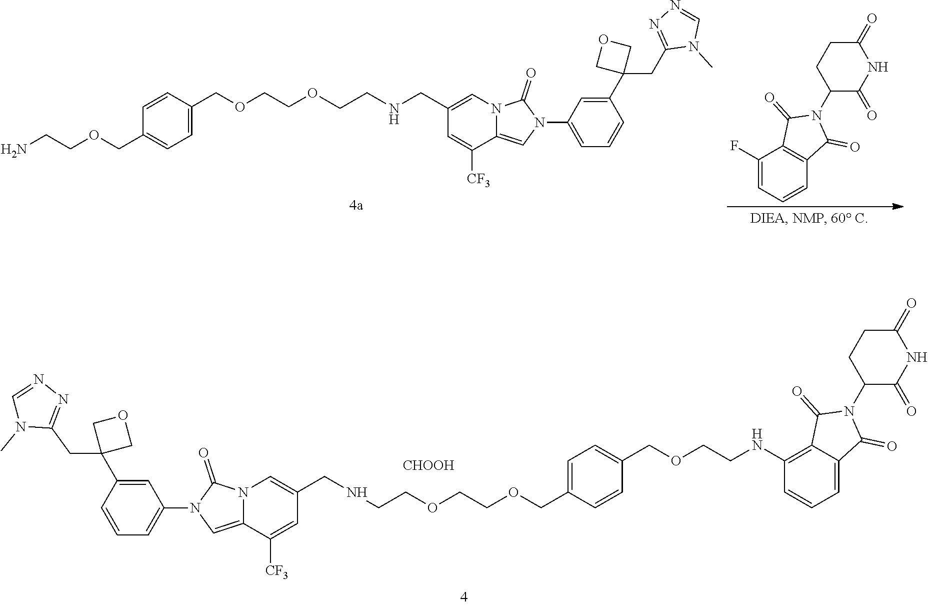

### Synthesis of Compound 4

To a stirred solution of Compound 4a (90 mg, 0.127 mmol, 1 equiv) and 2-(2,6-dioxopiperidin-3-yl)-4-fluoroisoindole-1,3-dione (105.08 mg, 0.381 mmol, 3 equiv) in NMP (0.5 mL) was added DIEA (49.16 mg, 0.381 mmol, 3 equiv). The resulting mixture was stirred for overnight at 60° C. The reaction solution was purified by Prep-HPLC with the following conditions (Column: XSelect CSH Prep C18 OBD Column, 19*250 mm, 5 μm; Mobile Phase A: Water (0.1% FA), Mobile Phase B: ACN; Flow rate: 30 mL/min; Gradient: 26% B to 36% B in 8 min, 36% B; Wave Length: 254; 220 nm; RT1 (min): 7.50; Number Of Runs: 0) to afford Compound 4 (18 mg, 14.03%) as a yellow solid.

LCMS-Compound 4: (ES, m/z): [M−HCOOH+H]⁺ 964

NMR-Compound 4: (400 MHz, CD₃OD, δ ppm): 62.13-2.16 (m, 1H), 2.72-2.79 (m, 2H), 2.80-2.88 (m, 1H), 3.09-3.15 (m, 2H), 3.48-3.50 (t, 2H), 3.67-3.75 (m, 10H), 3.84-3.87 (m, 2H), 4.53-4.55 (d, 4H), 5.06-5.10 (m, 5H), 6.92-6.94 (d, 1H), 3.99-7.02 (m, 2H), 7.06 (s, 1H), 7.14 (s, 1H), 7.31-7.32 (m, 5H), 7.44-7.50 (m, 2H), 7.61-7.63 (m, 1H), 7.81 (s, 1H), 8.20 (s, 1H), 8.50 (brs, 1H).

### Example 5. Compound 5

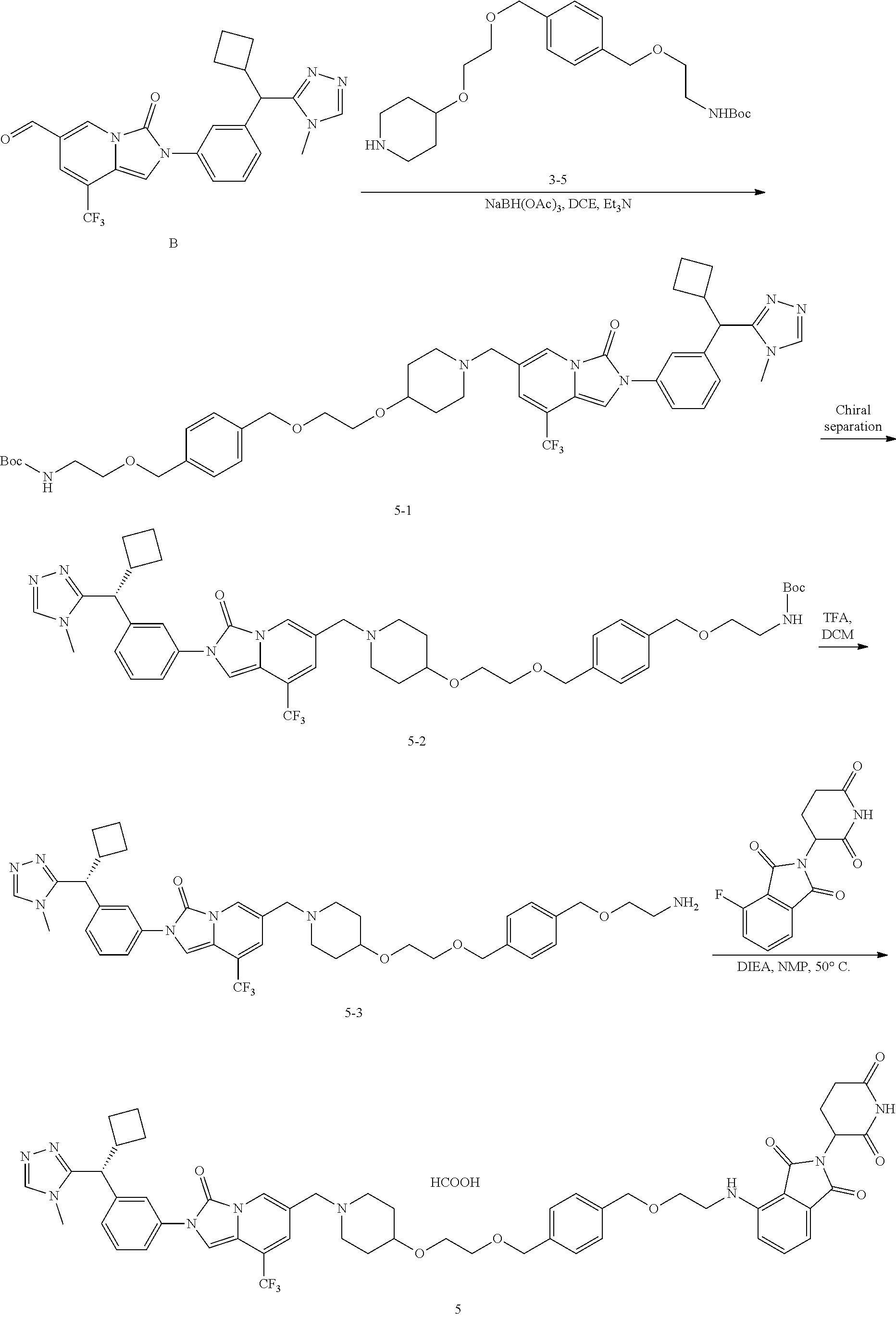

### Synthesis of Compound 5-1

To a stirred solution of Intermediate B (600 mg, 1.31 mmol, 1.0 equiv) and Compound 3-5 (1076 mg, 2.64 mmol, 2.0 equiv) in DCE were added Et3N (399 mg, 3.95 mmol, 3.0 equiv) and NaBH(OAc)3 (558 mg, 2.64 mmol, 2.0 equiv) at room temperature. The reaction solution mixture was stirred for overnight at room temperature. The residue was purified by Prep-TLC (CH2Cl2/MeOH=10:1) to afford Compound 5-1 (400 mg, 34%) as a yellow solid.

### Synthesis of Compound 5-2

The Compound 5-1 (400 mg) was purified by Prep-CHIRAL-HPLC with the following conditions (Column: CHIRALPAK IC, 2*25 cm, 5 μm; Mobile Phase A: Hex (0.5% 2M NH3-MeOH)—HPLC, Mobile Phase B: EtOH:DCM=1:1—HPLC; Flow rate: 20 mL/min; Gradient: 75% B to 75% B in 7 min; Wave Length: 220/254 nm; RT1 (min): 4.87) to afford Compound 5-2 (130 mg, 32.50%) as a yellow solid.

### Synthesis of Compound 5-3

A solution of Compound 5-2 (130 mg, 0.15 mmol, 1.0 equiv) and TFA (0.3 mL) in DCM (1 mL) was stirred for overnight at room temperature. The reaction solution was purified by Prep-TLC (CH2Cl2/MeOH=10:1) to afford Compound 5-3 (110 mg, 96%) as a yellow solid.

### Synthesis of Compound 5

To a stirred solution of Compound 5-3 (120 mg, 0.16 mmol, 1.0 equiv) and 2-(2,6-dioxopiperidin-3-yl)-4-fluoroisoindole-1,3-dione (132 mg, 0.48 mmol, 3.0 equiv) in NMP (1.5 mL) was added DIEA (621 mg, 0.48 mmol, 3.0 equiv) dropwise at room temperature. The resulting mixture was stirred for overnight at 50° C. The reaction solution (100 mg) was purified by Prep-HPLC with the following conditions (Column: XBridge BEH C18 OBD Prep Column, 19*250 mm, 5 μm; Mobile Phase A: Water (0.1% FA), Mobile Phase B: ACN; Flow rate: 30 mL/min; Gradient: 39% B to 50% B in 7 min, 50% B; Wave Length: 254; 220 nm; RT1 (min): 6.0) to afford Compound 5 (6.5 mg, 3.67%) as a yellow solid.

LC-MS-Compound 5: (ES, m/z): [M-COOH]+ 1004

H-NMR-Compound 5: (400 MHz, MeOD, δ ppm): 1.75-1.77 (m, 3H), 1.91-1.95 (m, 7H), 2.09-2.13 (m, 1H), 2.19-2.30 (m, 1H), 2.50 (s, 2H), 2.74-2.78 (m, 3H), 2.84-2.89 (m, 3H), 3.45-3.50 (m, 5H), 3.50-3.55 (m, 3H), 3.64-3.65 (m, 4H), 3.71-3.73 (m, 2H), 4.28-4.31 (d, 1H), 4.55-4.58 (m, 4H), 5.06-5.10 (m, 1H), 6.99-7.01 (d, 2H), 7.08-7.11 (d, 2H), 7.27-7.29 (m, 1H), 7.33-7.38 (m, 5H), 7.45-7.52 (m, 2H), 7.63-7.66 (m, 2H), 7.71 (s, 1H), 8.38 (s, 4H).

### Example 6. Compound 6

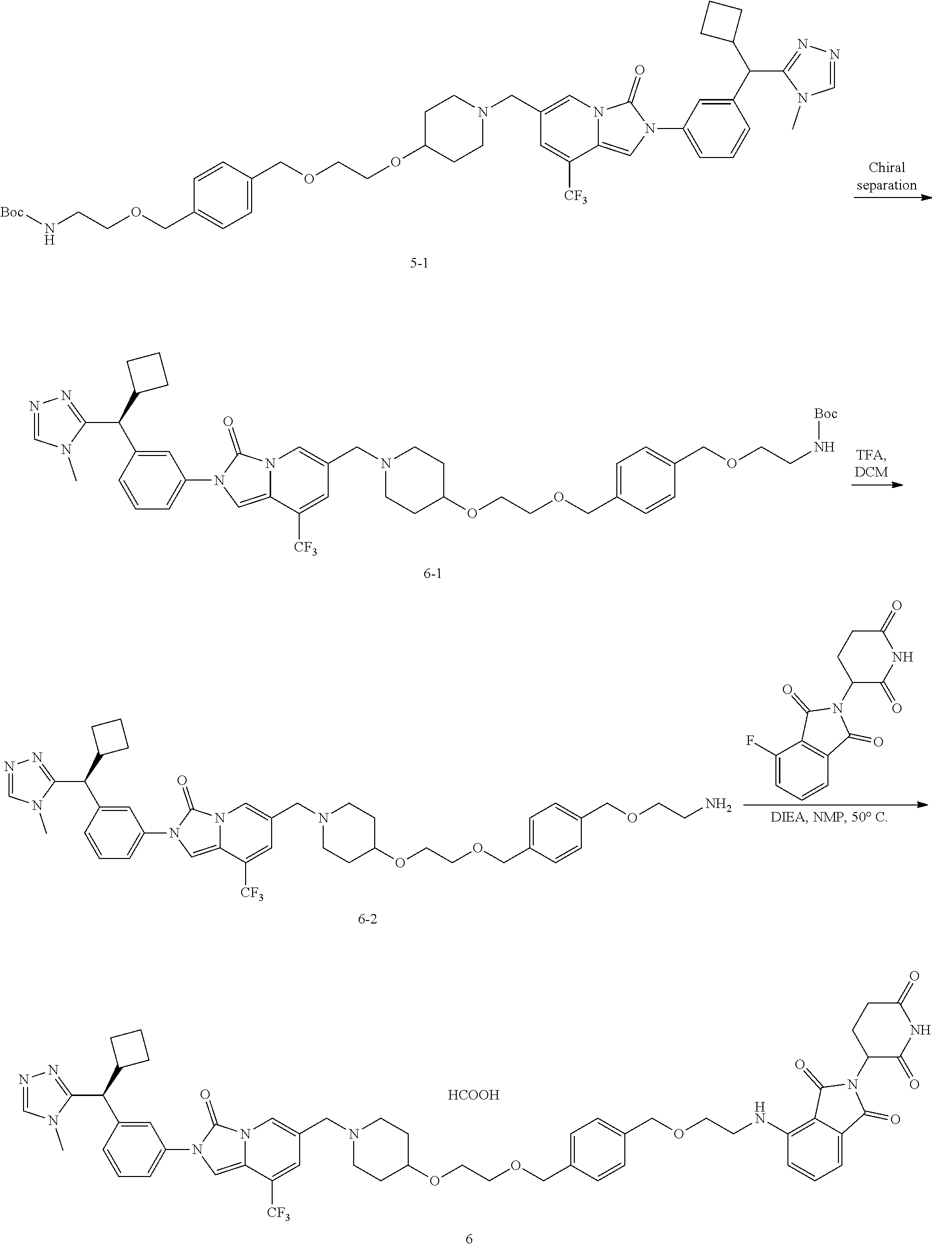 [SMILES: Cn1cnnc1C(c1cccc(-n2cc3c(C(F)(F)F)cc(CN4CCC(OCCOCc5ccc(COCCNC(=O)OC(C)(C)C)cc5)CC4)cn3c2=O)c1)C1CCC1 ; Cn1cnnc1[C@H](c1cccc(-n2cc3c(C(F)(F)F)cc(CN4CCC(OCCOCc5ccc(COCCNC(=O)OC(C)(C)C)cc5)CC4)cn3c2=O)c1)C1CCC1 ; Cn1cnnc1[C@H](c1cccc(-n2cc3c(C(F)(F)F)cc(CN4CCC(OCCOCc5ccc(COCCN)cc5)CC4)cn3c2=O)c1)C1CCC1 ; O=C1CCC(N2C(=O)c3cccc(F)c3C2=O)C(=O)N1 ; Cn1cnnc1[C@H](c1cccc(-n2cc3c(C(F)(F)F)cc(CN4CCC(OCCOCc5ccc(COCCNc6cccc7c6C(=O)N(C6CCC(=O)NC6=O)C7=O)cc5)CC4)cn3c2=O)c1)C1CCC1 ; "HCOOH"]

### Synthesis of Compound 6-1.

The Compound 5-1 (400 mg) was purified by Chiral-Prep-HPLC with the following conditions (Column: CHIRALPAK IC, 2*25 cm, 5 μm; Mobile Phase A: Hex (0.5% 2M NH₃-MeOH)—HPLC, Mobile Phase B: EtOH:DCM=1:1—HPLC; Flow rate: 20 mL/min; Gradient: 75% B to 75% B in 7 min; Wave Length: 220/254 nm; RT1 (min): 6.19) to afford Compound 6-1 (120 mg, 30%) as a yellow solid.

### Synthesis of Compound 6-2.

A solution of Compound 6-1 (120 mg, 0.14 mmol, 1.0 equiv) and TFA (0.3 mL) in DCM (1 mL) was stirred for overnight at room temperature under nitrogen atmosphere. The residue was purified by Prep-TLC (CH₂Cl₂/MeOH=10:1) to afford Compound 6-2 (90 mg, 85%) as a yellow solid.

### Synthesis of Compound 6.

To a stirred solution of Compound 6-2 (90 mg, 0.12 mmol, 1.0 equiv) and 2-(2,6-dioxopiperidin-3-yl)-4-fluoroisoindole-1,3-dione (99 mg, 0.36 mmol, 3.0 equiv) in NMP (1 mL) was added DIEA (46 mg, 0.36 mmol, 3.0 equiv) dropwise at room temperature. The resulting mixture was stirred for overnight at 50° C. The reaction solution (100 mg) was purified by Prep-HPLC with the following conditions (Column: XSelect CSH Prep C18 OBD Column, 19*250 mm, 5 μm; Mobile Phase A: Water (0.1% FA), Mobile Phase B: ACN; Flow rate: 30 mL/min; Gradient: 31% B to 41% B in 8 min, 41% B; Wave Length: 254; 220 nm; RT1 (min): 7.50) to afford Compound 6 (3 mg, 2%) as a yellow solid.

LC-MS-Compound 6: (ES, m/z): [M-COOH]⁺ 1004

H-NMR-Compound 6: (400 MHz, MeOD, δ ppm): 1.75-1.91 (m, 1OH), 2.12-2.26 (m, 2H), 2.54 (s, 2H), 2.70-2.89 (m, 5H), 3.50-3.55 (m, 8H), 3.64-3.73 (m, 6H), 4.28-4.31 (m, 1H), 4.55-4.58 (m, 4H), 5.06-5.09 (m, 1H), 6.99-7.01 (d, 2H), 7.07-7.11 (d, 2H), 7.27-7.52 (m, 8H), 7.63-7.71 (m, 3H), 8.38 (s, 4H).

### Example 7. Compound 7

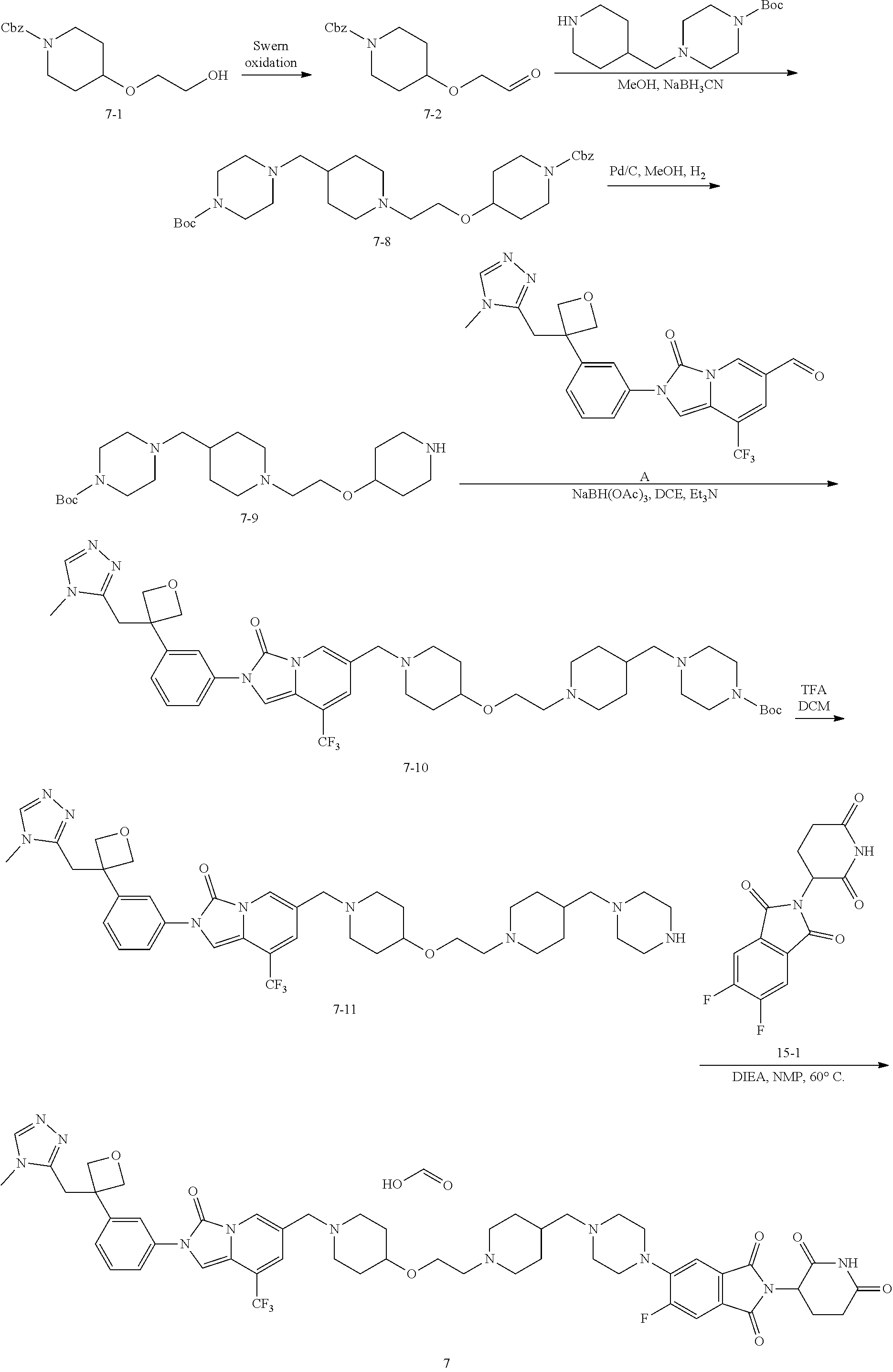

### Synthesis of Compound 7-2.

To a stirred solution of 7-1 (6.3 g, 22.553 mmol, 1 equiv) and Et3N (9.13 g, 90.212 mmol, 4 equiv) in DMSO (30 mL) were added pyridine; sulfonylideneoxidane (10.80 g, 67.885 mmol, 3.01 equiv) in DMSO (30 mL) dropwise at 0° C. The resulting mixture was stirred for 2 h at room temperature. The resulting mixture was diluted with water (200 mL). The resulting mixture was extracted with EtOAc (2×100 mL). The combined organic layers were washed with HCl (1 M, 50 mL) and brine (20 mL), dried over anhydrous Na2SO4. After filtration, the filtrate was concMentrated under reduced pressure. This resulted in Compound 7-2 (3 g, 47.97%) as a light yellow oil.

### Synthesis of Compound 7-8.

To a stirred solution of Compound 7-2 (3 g, 10.818 mmol, 1 equiv) and tert-butyl 4-(piperidin-4-ylmethyl)piperazine-1-carboxylate (3.07 g, 10.818 mmol, 1 equiv) in MeOH (50 mL) was added NaBH₃CN (0.82 g, 12.982 mmol, 1.2 equiv) under nitrogen atmosphere. The resulting mixture was stirred for overnight at room temperature under nitrogen atmosphere. The reaction was quenched by the addition of sat. NH₄Cl (aq.) (200 mL). The resulting mixture was extracted with CH₂Cl₂ (2×100 mL). The combined organic layers were concentrated under reduced pressure. The residue was purified by reverse flash chromatography with the following conditions: column, C18 silica gel; mobile phase, MeCN in Water (10 mmol/L NH₄HCO₃), 30% to 95% gradient in 30 min; detector, UV 220 nm. This resulted in Compound 7-8 (2 g, 33.94%) as a colorless oil.

### Synthesis of Compound 7-9.

To a solution of Compound 7-8 (2 g, 3.671 mmol, 1 equiv) in 50 mL MeOH was added Pd/C (400 mg, 10%) under nitrogen atmosphere in a 250 mL round-bottom flask. The mixture was hydrogenated at room temperature for overnight under hydrogen atmosphere using a hydrogen balloon, filtered through a Celite pad and concentrated under reduced pressure. This resulted in Compound 7-9 (1.8 g, crude) as a colorless oil.

### Synthesis of Compound 7-10.

To a stirred solution of Compound 7-9 (1.08 g, 2.623 mmol, 1.2 equiv) and Intermediate A (1 g, 2.186 mmol, 1.00 equiv) in DCE (20 mL) were added Et₃N (0.22 g, 2.186 mmol, 1 equiv) and NaBH(OAc)₃ (0.93 g, 4.372 mmol, 2 equiv). The resulting mixture was stirred for overnight at room temperature. The reaction was quenched by the addition of water (20 mL). The resulting mixture was extracted with CH₂Cl₂/MeOH=10/1 (2×30 mL). The combined organic layers were concentrated under reduced pressure. The residue was purified by reverse flash chromatography with the following conditions: column, C18 silica gel; mobile phase, MeCN in Water (10 mmol/L NH₄HCO₃), 20% to 85% gradient in 30 min; detector, UV 254 nm. This resulted in Compound 7-10 (500 mg, 26.84%) as a yellow solid.

### Synthesis of Compound 7-11.

To a stirred solution of Compound 7-10 (500 mg, 0.587 mmol, 1 equiv) in DCM (4 mL) were added TFA (1 mL). The resulting mixture was stirred for 3 h at room temperature. The resulting mixture was concentrated under reduced pressure. The residue was purified by Prep-TLC (CH₂Cl₂/MeOH 10:1) to afford Compound 7-11 (320 mg, 72.52%) as a yellow solid.

### Synthesis of Compound 7.

To a stirred solution of Compound 7-11 (200 mg, 0.266 mmol, 1 equiv) and Compound 15-1 (234.78 mg, 0.798 mmol, 3 equiv) in NMP (2 mL) was added DIEA (68.76 mg, 0.532 mmol, 2 equiv). The resulting mixture was stirred for overnight at room temperature. The crude product was purified by Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 6% B to 16% B in 7 min, 16% B; Wave Length: 254; 220 nm; RT1 (min): 6.3) to afford Compound 7 (114.9 mg, 40.29%) as a yellow solid.

LCMS-Compound 7: (ES, m/z): [M−HCOOH+H]⁺ 1026

NMR-Compound 7: (400 MHz, CD₃OD, δ ppm): 1.47-1.53 (m, 2H), 1.73-1.76 (m, 2H), 1.93-2.16 (m, 6H), 2.37-2.39 (d, 2H), 2.45-2.53 (m, 2H), 2.68-2.78 (m, 7H), 2.85-2.97 (m, 6H), 3.02-3.10 (m, 2H), 3.30-3.32 (m, 5H), 3.52-3.63 (m, 5H), 3.68 (s, 2H), 3.82-3.89 (m, 2H), 5.07-5.13 (m, 5H), 6.94-6.96 (m, 1H), 7.12-7.14 (m, 2H), 7.29 (s, 1H), 7.46-7.50 (m, 2H), 7.55-7.62 (m, 1H), 7.64-7.65 (d, 1H), 7.74 (s, 1H), 8.21 (s, 1H), 8.35 (s, 2H).

### Compound 8

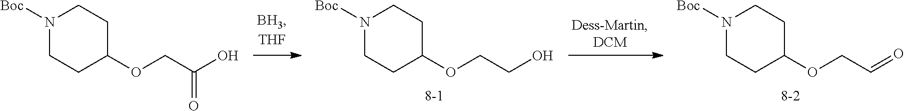 [SMILES: CC(C)(C)OC(=O)N1CCC(OCC(=O)O)CC1 ; CC(C)(C)OC(=O)N1CCC(OCCO)CC1 ; CC(C)(C)OC(=O)N1CCC(OCC=O)CC1]

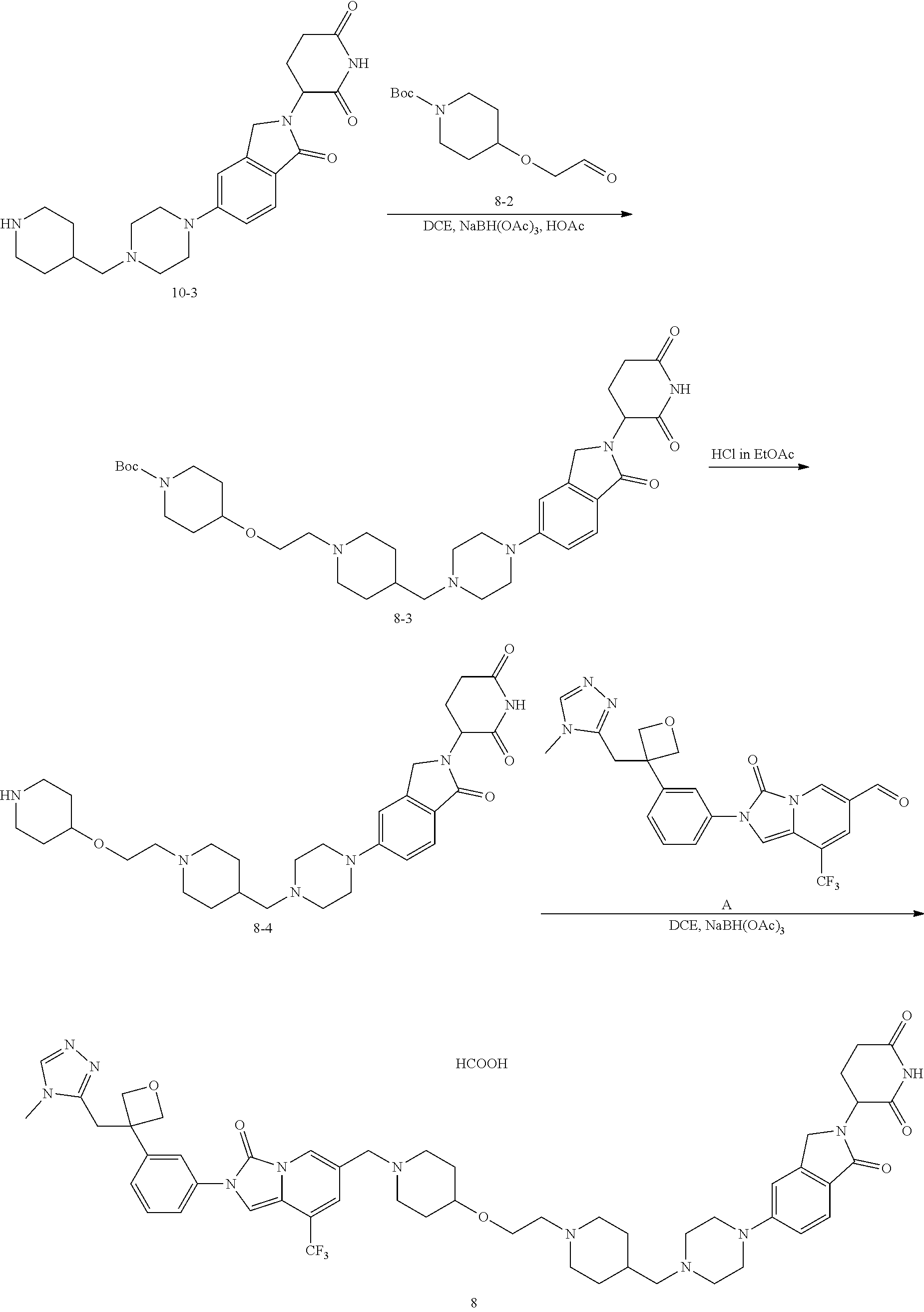

### Synthesis of Compound 8-1.

A solution of {[1-(tert-butoxycarbonyl)piperidin-4-yl]oxy}acetic acid (1 g, 3.857 mmol, 1 equiv) and BH3-THF (0.66 g, 7.714 mmol, 2 equiv) in THF (10 mL) was stirred for overnight at room temperature. The reaction was quenched with Water/Ice at 0° C. The resulting mixture was extracted with EtOAc (3×30 mL). The combined organic layers were washed with water (3×20 mL), dried over anhydrous Na2SO4. After filtration, the filtrate was concentrated under reduced pressure. This resulted in Compound 8-1 (900 mg, 95.13%) as a colorless oil.

### Synthesis of Compound 8-2.

A solution of Compound 8-1 (900 mg, 3.669 mmol, 1 equiv) and Dess-Martin (3112.10 mg, 7.338 mmol, 2 equiv) in DCM (10 mL) was stirred for 4 h at 0° C. The reaction was quenched with sat. NaHCO₃ (aq.) (20 mL) at room temperature. The resulting mixture was extracted with CH₂Cl₂ (3×20 mL). The combined organic layers were concentrated under reduced pressure. The residue was purified by Prep-TLC (PE/EA 5:1) to afford Compound 8-2 (580 mg, 64.98%) as a colorless oil.

### Synthesis of Compound 8-3.

To a stirred mixture of Compound 10-3 (800 mg, 1.732 mmol, 1 equiv) and Compound 8-2 (631.97 mg, 2.598 mmol, 1.5 equiv) in DCE (20 mL) was added Et₃N (350.46 mg, 3.464 mmol, 2 equiv) and NaBH(OAc)₃ (734.01 mg, 3.464 mmol, 2 equiv). The resulting mixture was stirred for 4 h at room temperature. The resulting mixture was diluted with H₂O/AcOH=10/1 (15 mL). The resulting mixture was extracted with CH₂Cl₂ (2×20 mL). The combined organic layers were concentrated under reduced pressure. The residue was purified by reverse flash chromatography with the following conditions: column, C18 silica gel; mobile phase, MeCN in water (0.1% HOAc), 5% to 90% gradient in 30 min; detector, UV 220 nm. This resulted in Compound 8-3 (420 mg, 37.15%) as a white solid.

### Synthesis of Compound 8-4.

To a stirred solution of Compound 8-3 (420 mg, 0.643 mmol, 1 equiv) in EtOAc (5 mL) was added HCl(gas) in EtOAc (0.64 mL, 2.572 mmol, 4 equiv) dropwise. The resulting mixture was stirred for overnight at room temperature. The precipitated solids were collected by filtration and washed with EtOAc (2 mL). The crude product (360 mg) was purified by reverse flash chromatography with the following conditions: column, C18 silica gel; mobile phase, MeCN in water (0.1% HOAc), 0% to 70% gradient in 30 min; detector, UV 220 nm. This resulted in Compound 8-4 (320 mg, 89.99%) as a white solid.

### Synthesis of Compound 8.

To a stirred solution of Compound 8-4 (100 mg, 0.181 mmol, 1 equiv) and Intermediate A (82.76 mg, 0.181 mmol, 1 equiv) in MeOH (2 mL) was added NaBH₃CN (11.37 mg, 0.181 mmol, 1 equiv) at room temperature. The resulting mixture was stirred for 2 h at room temperature. The reaction solution was purified by Prep-HPLC with the following conditions (Column: XSelect CSH Prep C18 OBD Column, 19*250 mm, 5 μm; Mobile Phase A: Water (0.1% FA), Mobile Phase B: ACN; Flow rate: 30 mL/min; Gradient: 3% B to 15% B in 9 min, 15% B; Wave Length: 254; 220 nm; RT1 (min): 8.45) to afford Compound 8 (26.4 mg, 14.03%) as a yellow solid.

LCMS-Compound 8: (ES, m/z): [M−HCOOH+H]⁺ 994

NMR-Compound 8: (400 MHz, CD₃OD, δ ppm): δ1.47-1.58 (m, 2H), 1.68-1.74 (m, 2H), 1.85-2.17 (m, 6H), 2.25-2.34 (m, 4H), 2.40-2.51 (m, 1H), 2.62-2.71 (m, 4H), 2.81-3.03 (m, 9H), 3.32 (s, 2H), 3.38-3.40 (m, 6H), 3.51-3.59 (m, 3H), 3.67 (s, 2H), 3.83 (s, 2H), 4.41-4.42 (m, 2H), 5.06-5.14 (m, 5H), 6.94-6.96 (d, 1H), 7.09-7.13 (m, 4H), 7.29 (s, 1H), 7.46-7.50 (t, 1H), 7.62-7.68 (m, 3H), 8.20 (s, 1H), 8.50 (brs, 1H).

### Example 8. Compound 9

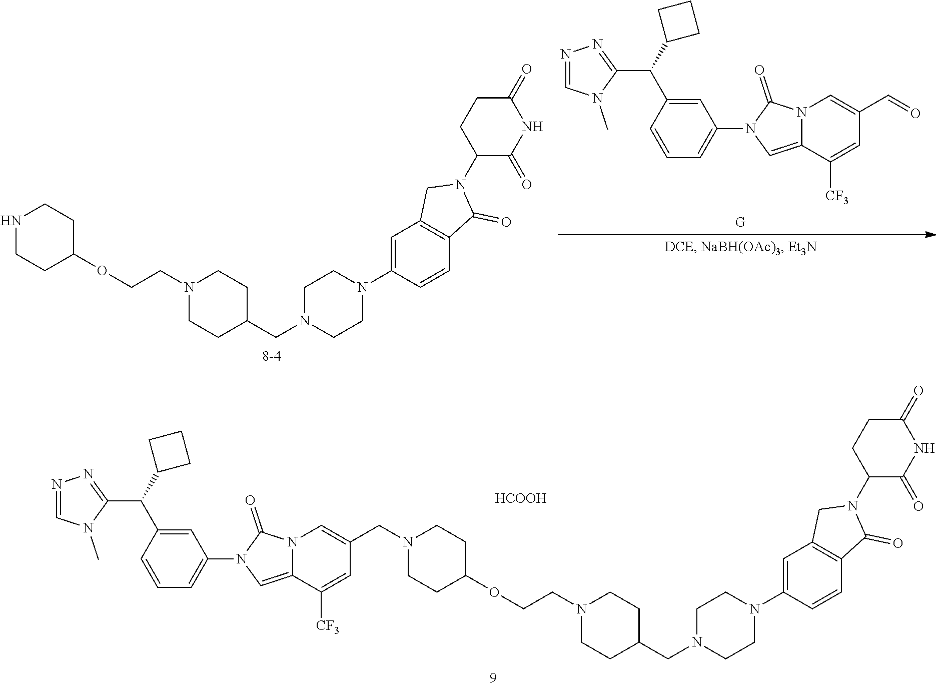

### Synthesis of Compound 9.

To a stirred solution of Compound 8-4 (120 mg, 0.204 mmol, 1 equiv) and Intermediate G (92.76 mg, 0.204 mmol, 1 equiv) in DCE (10 mL) was added Et₃N (20.61 mg, 0.204 mmol, 1 equiv) NaBH(OAc)₃ (86.33 mg, 0.408 mmol, 2 equiv). The resulting mixture was stirred for overnight at room temperature. The resulting mixture was diluted with water (5 mL). The resulting mixture was extracted with CH₂Cl₂/MeOH=10/1 (3×10 mL). The combined organic layers were concentrated under reduced pressure. The residue was purified by Prep-HPLC with the following conditions (Column XSelect CSH Prep C18 OBD Column, 19*250 mm, 5 μm; Mobile Phase A: Water (0.1% FA), Mobile Phase B: ACN; Flow rate: 30 mL/min; Gradient: 9% B to 19% B in 7 min, 19% B; Wave Length: 254; 220 nm; RT1 (min): 6.31) to afford Compound 9 (16 mg, 7.57%) as a yellow solid.

LCMS-Compound 9: (ES, m/z): [M−HCOOH+H]⁺ 993

NMR-Compound 9: (400 MHz, CD₃OD, δ ppm): δ1.45-1.53 (m, 2H), 1.55-1.74 (m, 3H), 1.89-2.29 (m, 12H), 2.31-2.55 (m, 5H), 2.63-2.69 (m, 4H), 2.75-2.82 (m, 1H), 2.83-2.96 (m, 3H), 3.00-3.12 (m, 2H), 3.40-3.42 (m, 2H), 3.42-3.44 (m, 4H), 3.47-3.59 (m, 8H), 3.84 (s, 2H), 4.29-4.31 (1, 2H), 4.35-4.42 (m, 2H), 5.09-5.13 (m, 1H), 7.09-7.14 (m, 4H), 7.30-7.32 (d, 1H), 7.49-7.53 (t, 1H), 7.64-7.67 (m, 3H), 7.71 (s, 1H), 8.25 (brs, 2H), 8.30 (s, 1H).

### Example 9. Compound 10

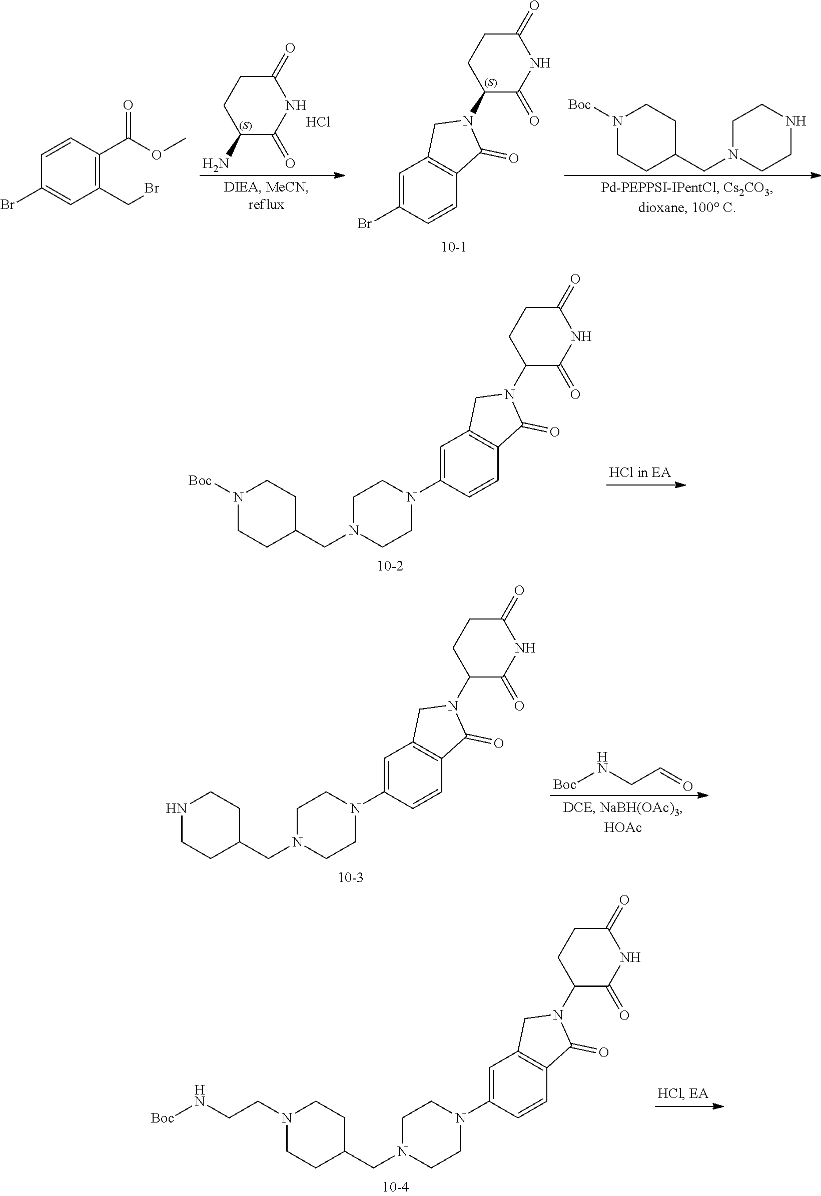

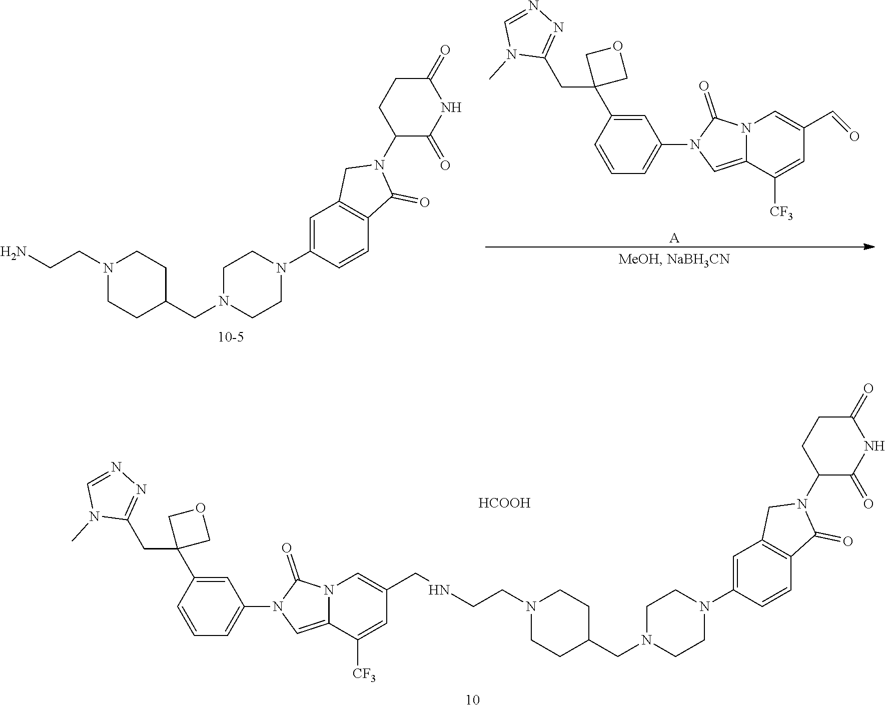

### Synthesis of Compound 10-1.

To a stirred mixture of methyl 4-bromo-2-(bromomethyl)benzoate (7 g, 22.730 mmol, 1 equiv) and (3S)-3-aminopiperidine-2,6-dione hydrochloride (3.74 g, 22.730 mmol, 1 equiv) in MeCN (150 mL) was added DIEA (6.46 g, 50.006 mmol, 2.2 equiv) dropwise. The resulting mixture was stirred for overnight at 80° C. under nitrogen atmosphere. The precipitated solids were collected by filtration and washed with MeCN (2×5 mL). The resulting solid was dried under vacuum. This resulted in Compound 10-1 (7 g, 95.30%) as a black solid.

### Synthesis of Compound 10-2.

To a stirred mixture of Compound 10-1 (1.87 g, 6.610 mmol, 1.2 equiv) in dioxane (40 mL) was added Cs₂CO₃ (5.38 g, 16.524 mmol, 3 equiv) and Pd PEPPSI IPentCl (0.47 g, 0.551 mmol, 0.1 equiv) under nitrogen atmosphere. The resulting mixture was stirred for 4 h at 100° C. under nitrogen atmosphere. The reaction was quenched by the addition of Water/HOAc=10/1 (120 mL). The resulting mixture was extracted with CH₂Cl₂ (4×100 mL). The combined organic layers were concentrated under reduced pressure. The residue was purified by silica gel column chromatography, eluted with CH₂Cl₂/MeOH (10:1) to afford Compound 10-2 (1.5 g, 51.81%) as a grey solid.

### Synthesis of Compound 10-3.

To a stirred solution of Compound 10-2 (1.48 g, 2.816 mmol, 1 equiv) in EtOAc (20 mL) was added HCl(g) in ETOAc (2.82 mL, 11.264 mmol, 4 equiv). The resulting mixture was stirred for overnight at room temperature. The precipitated solids were collected by filtration and washed with EtOAc (2×5 mL). The resulting solid was dried under vacuum. This resulted in Compound 10-3 (1.2 g, 92.25%) as a white solid.

### Synthesis of Compound 10-4.

To a stirred mixture of Compound 10-3 (1.2 g, 2.820 mmol, 1 equiv) and tert-butyl N-(2-oxoethyl)carbamate (0.90 g, 5.640 mmol, 2 equiv) in DCE (15 mL) was added Et₃N (0.29 g, 2.820 mmol, 1 equiv) and NaBH(OAc)₃ (1.20 g, 5.640 mmol, 2 equiv). The resulting mixture was stirred for overnight at room temperature under nitrogen atmosphere. The reaction was quenched by the addition of water (25 mL). The resulting mixture was extracted with CH₂Cl₂ (2×20 mL). The combined organic layers were concentrated under reduced pressure. The residue was purified by reverse flash chromatography with the following conditions: column, C18 silica gel; mobile phase, MeCN in water (10 mmoL/L NH₄HCO₃), 35% to 100% gradient in 40 min; detector, UV 220 nm. This resulted in Compound 10-4 (650 mg, 40.53%) as a white solid.

### Synthesis of Compound 10-5.

To a stirred solution of Compound 10-4 (650 mg, 1.143 mmol, 1 equiv) in DCM (1.5 mL) was added TFA (6 mL). The resulting mixture was stirred for 4 h at room temperature. The resulting mixture was concentrated under reduced pressure. The residue was purified by reverse flash chromatography with the following conditions: column, C18 silica gel; mobile phase, MeCN in water (10 mmoL/L NH₄HCO₃), 0% to 70% gradient in 25 min; detector, UV 220 nm. This resulted in Compound 10-5 (500 mg, 93.36%) as a light yellow solid.

### Synthesis of Compound 10.

To a stirred solution of Compound 10-5 (150 mg, 0.320 mmol, 1 equiv) and Intermediate A (146.42 mg, 0.320 mmol, 1 equiv) in MeOH (3 mL) was added NaBH₃CN (20.12 mg, 0.320 mmol, 1 equiv). The resulting mixture was stirred for 2 h at room temperature. The reaction solution was purified by reverse flash chromatography with the following conditions: column, (C18 silica gel; mobile phase, MeCN in water (0.1% HOAc), 5% to 70% gradient in 25 min; detector, UV 254 nm. The crude product (70 mg) was purified by Prep-HPLC with the following conditions (Column: XSelect CSH Prep C18 OBD Column, 19*250 mm, 5 μm; Mobile Phase A: Water (0.1% FA), Mobile Phase B: ACN; Flow rate: 30 mL/min; Gradient: 28% B to 42% B in 10 min, 42% B; Wave Length: 254; 220 min; RT1 (min): 9.40) to afford Compound 10 (4.5 mg, 1.47%) as a yellow solid.

LCMS-Compound 10: (ES, m/z): [M−HCOOH+H]⁺ 910

NMR-Compound 10: (400 MHz, CD₃OD, δ ppm): δ1.58-1.73 (m, 2H), 2.01-2.08 (m, 2H), 2.12-2.27 (m, 3H), 2.41-2.53 (m, 1H), 2.78-2.82 (m, 3H), 2.88-3.01 (m, 5H), 3.15-3.22 (m, 5H), 3.42-3.64 (m, 7H), 3.67-3.86 (m, 6H), 4.07 (s, 2H), 444-4.46 (m, 2H), 5.06-5.16 (m, 6H), 6.96-7.08 (m, 1H), 7.17-7.22 (m, 4H), 7.30 (s, 1H), 7.49-7.52 (t, 1H), 7.62-7.63 (d, 1H), 7.71-7.73 (d, 1H), 7.95 (s, 1H), 8.31 (s, 1H).

### Compound 11

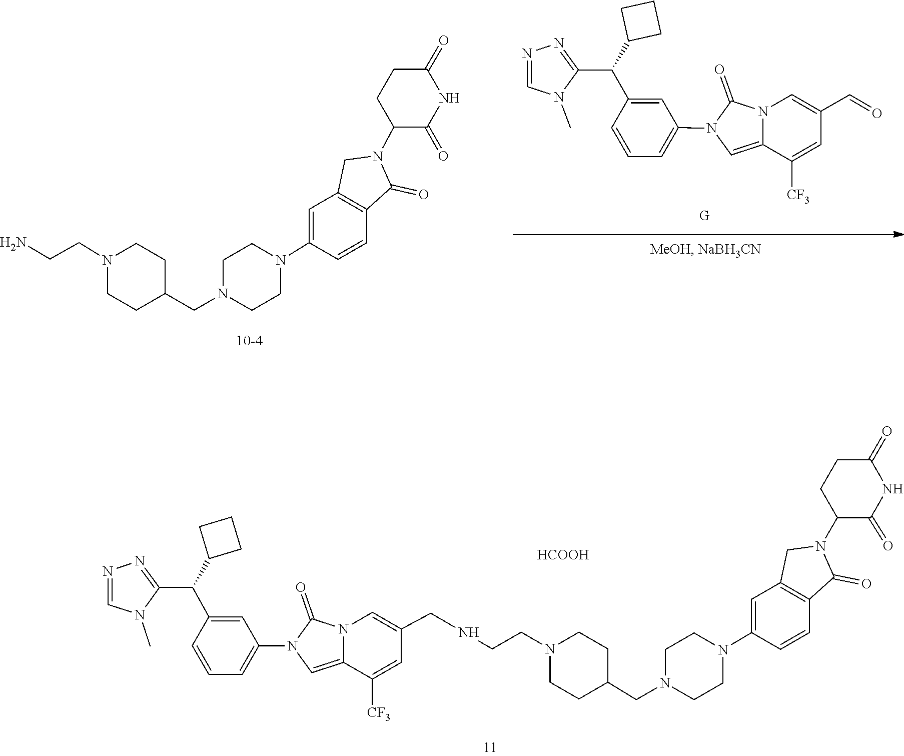 [SMILES: NCCN1CCC(CN2CCN(c3ccc4c(c3)CN(C3CCC(=O)NC3=O)C4=O)CC2)CC1 ; Cn1cnnc1[C@@H](c1cccc(-n2cc3c(C(F)(F)F)cc(C=O)cn3c2=O)c1)C1CCC1 ; Cn1cnnc1[C@@H](c1cccc(-n2cc3c(C(F)(F)F)cc(CNCCN4CCC(CN5CCN(c6ccc7c(c6)CN(C6CCC(=O)NC6=O)C7=O)CC5)CC4)cn3c2=O)c1)C1CCC1 ; "HCOOH"]

### Synthesis of Compound 11.

To a stirred solution of Compound 10-4 (100 mg, 0.213 mmol, 1 equiv) and Intermediate G (97.19 mg, 0.213 mmol, 1 equiv) in MeOH (2 mL) was added NaBH₃CN (13.41 mg, 0.213 mmol, 1 equiv). The resulting mixture was stirred for 2 h at room temperature. The reaction solution was purified by Prep-HPLC with the following conditions (Column: XSelect CSH Prep C18 OBD Column, 19*250 mm, 5 μm; Mobile Phase A: Water (0.1% FA), Mobile Phase B: ACN; Flow rate: 30 mL/min; Gradient: 28% B to 42% B in 8 min, 42% B; Wave Length: 254; 220 nm; RT1 (min): 7.38) to afford Compound 11 (11.2 mg, 5.50%) as a yellow solid.

LCMS-Compound 11: (ES, m/z): [M−HCOOH+H]⁺ 908

NMR-Compound 11: (400 MHz, CD₃OD, δ ppm): δ1.30-1.49 (m, 2H), 1.77-1.82 (m, 1H), 1.83-1.91 (m, 5H), 1.94-2.05 (m, 1H), 2.11-2.18 (m, 1H), 2.20-2.26 (m, 1H), 2.34-2.35 (d, 2H), 2.42-2.53 (m, 1H), 2.59 (s, 2H), 2.63-2.71 (m, 4H), 2.74-3.12 (m, 8H), 3.40-3.45 (m, 4H), 3.46-3.56 (m, 4H), 3.72 (s, 1H), 4.28-4.43 (m, 3H), 5.09-5.14 (m, 1H), 7.09-7.16 (m, 3H), 7.30-7.64 (m, 3H), 7.66-7.77 (m, 3H), 8.35 (s, 1H).

### Example 10. Compound 12

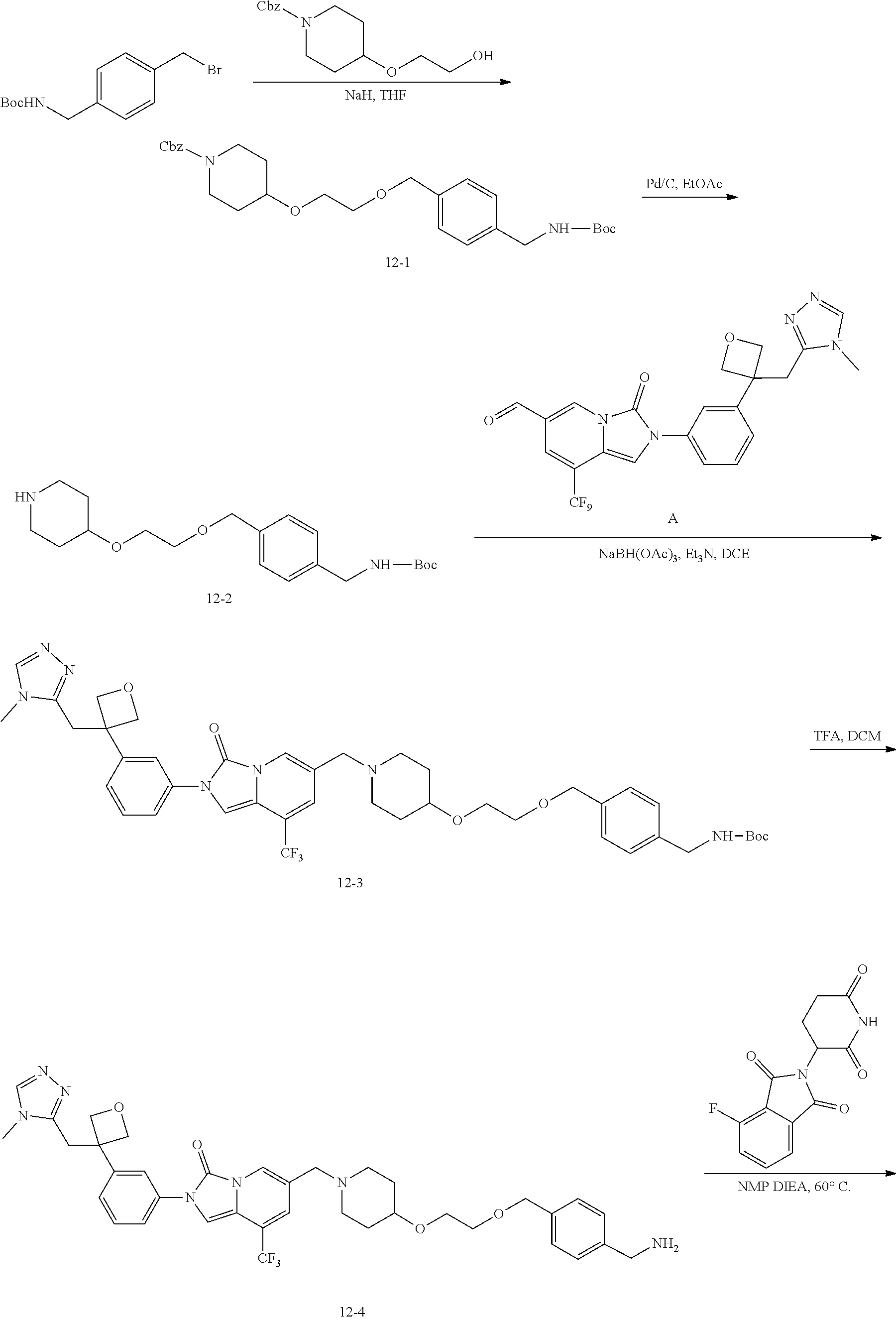

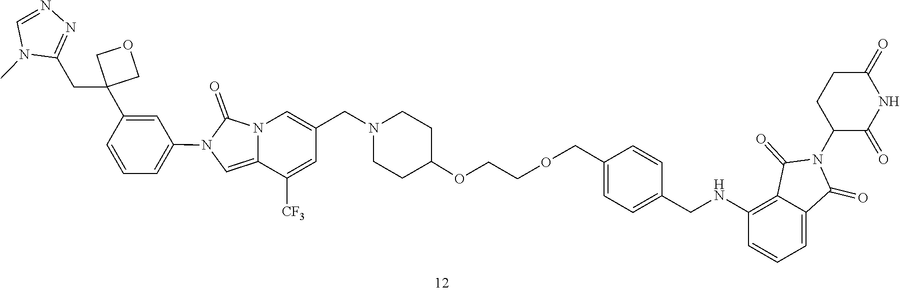 [SMILES: Cn1cnnc1CC1(c2cccc(-n3cc4c(C(F)(F)F)cc(CN5CCC(OCCOCc6ccc(CNc7cccc8c7C(=O)N(C7CCC(=O)NC7=O)C8=O)cc6)CC5)cn4c3=O)c2)COC1]

### Synthesis of Compound 12-1.

To a solution of benzyl 4-(2-hydroxyethoxy) piperidine-1-carboxylate (2.8 g, 9.99 mmol, 1.5 equiv) in THF (20 mL) was added sodium hydride (60% in oil, 190 mg) at 0 degrees C. The mixture was stirred for 30 min. tert-butyl N-{[4-(bromomethyl) phenyl] methyl} carbamate (2 g, 6.66 mmol, 1.0 equiv) was added and the mixture was allowed to warm to room temperature and stirred for overnight. The reaction mixture was quenched by water (50 mL) and extracted with DCM (3×25 mL). The residue was purified by silica gel column chromatography, eluted with PE/EA (1:1) to afford Compound 12-1 (1.5 g, 42%) as a colorless oil.

### Synthesis of Compound 12-2.

To a solution of Compound 12-1 (1.5 g, 3.00 mmol, 1 equiv) in 45 mL EtOAc was added Pd/C (10%, 300 mg) under nitrogen atmosphere in a 100 mL sealed tube. The mixture was hydrogenated at room temperature for 2 h under hydrogen atmosphere using a hydrogen balloon, filtered through a Celite pad and concentrated under reduced pressure. This resulted in Compound 12-2 (900 mg, 82%) as a colorless oil.

### Synthesis of Compound 12-3.

To a stirred solution of Intermediate A (880 mg, 1.92 mmol, 1.0 equiv) and Compound 12-1 (1402 mg, 3.84 mmol, 2.0 equiv) in DCE (8.8 mL) were added Et₃N (194 ng, 1.92 mmol, 1.0 equiv) and NaBH(OAc)₃ (815 mg, 3.84 mmol, 2.0 equiv) at room temperature. The resulting mixture was stirred for overnight at room temperature. The reaction was quenched with water (40 mL) at room temperature. The resulting mixture was extracted with CH₂Cl₂ (3×20 mL). The combined organic layers were washed with water (3×20 mL), dried over anhydrous Na₂SO₄. After filtration, the filtrate was concentrated under reduced pressure. The residue was purified by Prep-TLC (CH₂Cl₂/MeOH=10:1) to afford Compound 12-3 (430 mg, 28%) as a yellow solid.

### Synthesis of Compound 12-4.

A solution of Compound 12-3 (430 mg, 0.53 mmol, 1.0 equiv) and TFA (1 mL) in DCM (4 mL) was stirred for overnight at room temperature. The resulting mixture was concentrated under reduced pressure. The residue was purified by Prep-TLC (CH₂Cl₂/MeOH=10:1) to afford Compound 12-4 (260 mg, 69%) as a yellow solid.

### Synthesis of Compound 12.

To a stirred solution of Compound 12-4 (250 mg, 0.35 mmol, 1.0 equiv) and 2-(2,6-dioxopiperidin-3-yl)-4-fluoroisoindole-1,3-dione (293 mg, 1.06 mmol, 3.0 equiv) in NMP (2.5 mL) was added DIEA (137 mg, 1.06 mmol, 3.0 equiv) at room temperature. The resulting mixture was stirred for overnight at 60° C. The reaction solution was purified by Prep-HPLC with the following conditions (Column: Xselect CSH C18 OBD Column 30*150 mm 5 μm, n; Mobile Phase A: water (0.1% FA), Mobile Phase B: ACN; Flow rate: 60 mL/min; Gradient: 23% B to 33% B in 7 min, 33% B; Wave Length: 254; 220 nm; RT1 (min): 6.89) to afford Compound 12 (16 mg, 4%) as a yellow solid.

LC-MS-Compound 12: (ES, m/z): [M-COOH]⁺ 962

H-NMR-Compound 12: (400 MHz, DMSO-d6, δ ppm): 1.46 (s, 2H), 1.81 (s, 2H), 2.04-2.06 (m, 1H), 2.21 (s, 2H), 2.67 (s, 2H), 2.70-2.72 (m, 2H), 2.86-2.91 (m, 4H), 2.97 (s, 3H), 3.53 (s, 6H), 4.46-4.56 (m, 4H), 4.91-5.09 (m, 5H), 6.88-6.96 (m, 2H), 7.01-7.02 (m, 2H), 7.22-7.42 (m, 8H), 7.48-7.52 (m, 1H), 7.69-7.76 (m, 2H), 8.14-8.20 (m, 1H), 11.11 (s, 1H).

### Example 11. Compound 13

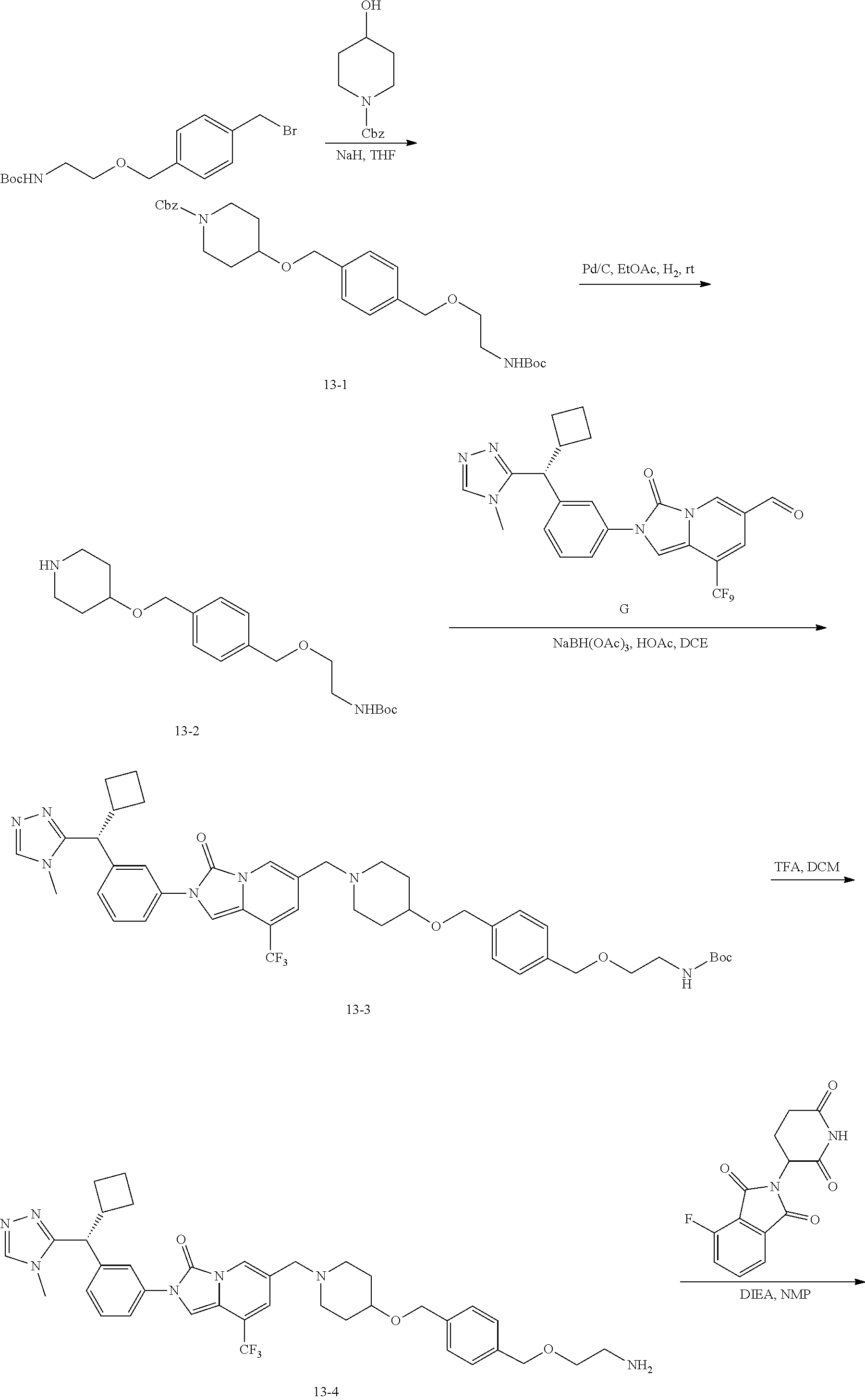

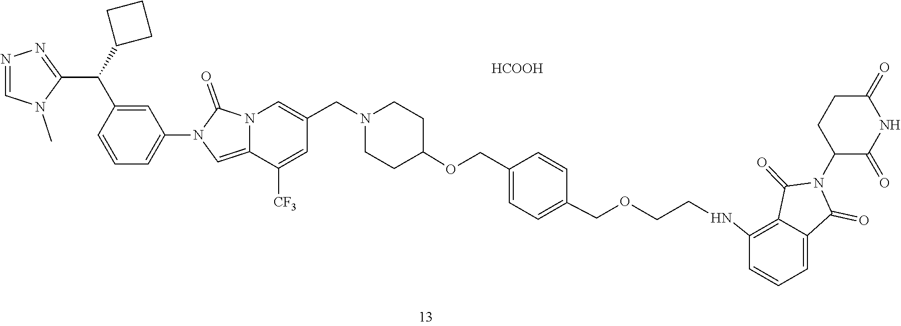 [SMILES: Cn1cnnc1[C@@H](c1cccc(-n2cc3c(C(F)(F)F)cc(CN4CCC(OCc5ccc(COCCNc6cccc7c6C(=O)N(C6CCC(=O)NC6=O)C7=O)cc5)CC4)cn3c2=O)c1)C1CCC1 ; "HCOOH"]

### Synthesis of Compound 13-1.

To a solution of benzyl 4-hydroxypiperidine-1-carboxylate (2 g, 8.71 mmol, 1.5 equiv) in THF (20 mL) was added sodium hydride (60% in oil, 278 mg) at 0 degrees C. The mixture was stirred for 1 h. tert-butyl N-(2-{[4-(bromomethyl) phenyl] methoxy} ethyl) carbamate (2 g, 5.81 mmol, 1.0 equiv) was added and the mixture was allowed to warm to room temperature and stirred for overnight. The reaction mixture was quenched by water (50 mL) and extracted with DCM (3*50 mL). The resulting mixture was concentrated under vacuum. The residue was purified by silica gel column chromatography, eluted with PE/EA (1:1) to afford Compound 13-1 (1.2 g, 39%) as a colorless oil.

### Synthesis of Compound 13-2.

To a solution of Compound 13-1 (1.2 g, 2.40 mmol, 1.0 equiv) in 36 mL EtOAc was added Pd/C (10%, 200 mg) under nitrogen atmosphere in a 100 mL round-bottom flask. The mixture was hydrogenated at room temperature for 2 h under hydrogen atmosphere using a hydrogen balloon, filtered through a Celite pad and concentrated under reduced pressure. The resulting mixture was concentrated under reduced pressure. This resulted in Compound 13-2 (400 mg, 46%) as a colorless oil.

### Synthesis of Compound 13-3.

To a stirred solution of Intermediate G (380 mg, 0.83 mmol, 1.0 equiv) and Compound 13-2 (608 mg, 1.66 mmol, 2.0 equiv) in DCE (4 mL) was added TEA (84 mg, 0.83 mmol, 1.0 equiv) at room temperature. The resulting mixture was stirred for 1 h at room temperature. To the above mixture was added NaBH(OAc)₃ (353.67 mg, 1.668 mmol, 2 equiv). The resulting mixture was stirred for additional overnight at room temperature. The reaction was quenched with water (20 mL) at room temperature. The resulting mixture was extracted with CH₂Cl₂ (3×20 mL). The combined organic layers were washed with water (3×20 mL), dried over anhydrous Na₂SO₄. After filtration, the filtrate was concentrated under reduced pressure. The residue was purified by Prep-TLC (CH₂Cl₂/MeOH=10:1) to afford Compound 13-3 (270 mg, 40%) as a yellow solid.

### Synthesis of Compound 13-4.

A solution of Compound 13-3 (270 mg, 0.33 mmol, 1.0 equiv) and TFA (0.5 mL) in DCM (2 mL) was stirred for overnight at room temperature. The resulting mixture was concentrated under reduced pressure. The residue was neutralized to pH 7 with NH₃ in MeOH. The residue was purified by Prep-TLC (CH₂Cl₂/MeOH=10:1) to afford Compound 13-4 (130 mg, 55%) as a yellow solid.

### Synthesis of Compound 13.

To a stirred solution of Compound 13-4 (120 mg, 0.17 mmol, 1.0 equiv) and 2-(2,6-dioxopiperidin-3-yl)-4-fluoroisoindole-1,3-dione (141 mg, 0.51 mmol, 3.0 equiv) in NMP (1.2 mL) was added DIEA (66 mg, 0.51 mmol, 3.0 equiv) at room temperature. The resulting mixture was stirred for overnight at 60° C. The reaction solution was purified by Prep-HPLC with the following conditions (Column: Xselect CSH C18 OBD Column 30*150 mm 5 μm, n; Mobile Phase A: water (0.1% FA), Mobile Phase B: ACN; Flow rate: 60 mL/min; Gradient: 28% B to 39% B in 7 min, 39% B; Wave Length: 254; 220 nm; RT1 (min): 6.97) to afford Compound 13 (15 mg, 8%) as a yellow solid.

LC-MS-Compound 13: (ES, m/z): [M-COOH]⁺ 960

H-NMR-Compound 13: (400 MHz, DMSO-d6, δ ppm): 1.49-1.52 (m, 2H), 1.75-1.84 (m, 8H), 2.05-2.14 (m, 4H), 2.67-2.68 (m, 1H), 2.81-2.82 (m, 1H), 3.21-3.27 (m, 5H), 3.39 (s, 3H), 3.41-3.43 (m, 2H), 3.61-3.64 (m, 2H), 4.25-4.27 (m, 1H), 4.47-4.52 (d, 4H), 5.06-5.10 (m, 1H), 6.65-6.67 (t, 1H), 7.02-7.05 (m, 2H), 7.13-7.20 (m, 2H), 7.29-7.31 (m, 5H), 7.42-7.46 (t, 1H), 7.55-7.59 (m, 1H), 7.66-7.74 (m, 3H), 8.33 (s, 1H), 11.01 (s, 1H).

### Example 12. Compound 14

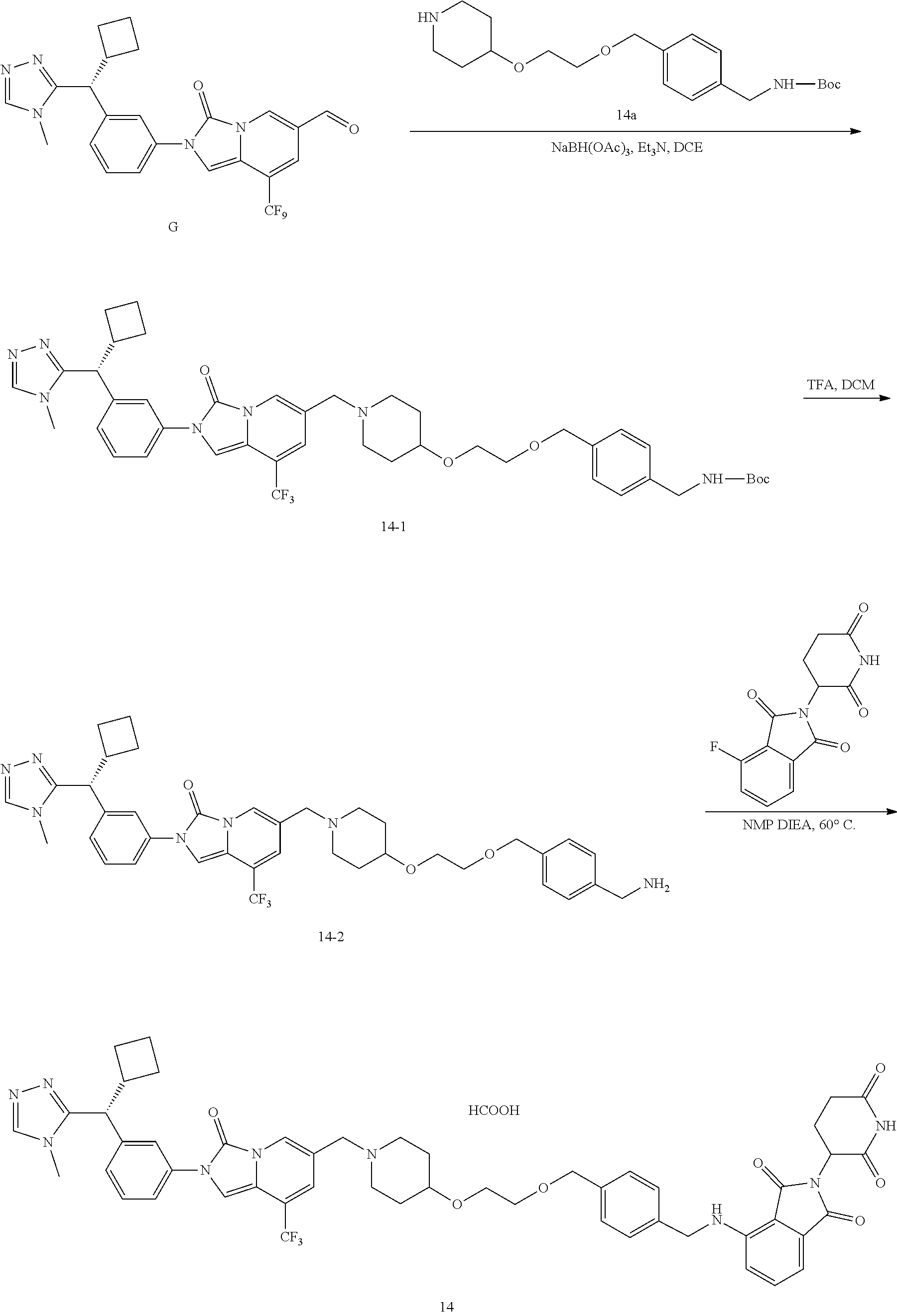

### Synthesis of Compound 14-1.

To a stirred solution of Intermediate G (500 mg, 1.09 mmol, 1.0 equiv) and Compound 14a (800 mg, 2.19 mmol, 2.0 equiv) in DCE (5 mL) were added Et₃N (111 mg, 1.09 mmol, 1.0 equiv) and NaBH(OAc)₃ (465 mg, 2.19 mmol, 2.0 equiv) at room temperature. The resulting mixture was stirred for overnight at room temperature. The reaction was quenched with water (20 mL) at room temperature. The resulting mixture was extracted with CH₂Cl₂ (3×20 mL). The combined organic layers were concentrated under reduced pressure. The residue was purified by Prep-TLC (CH₂Cl₂MeOH=10:1) to afford Compound 14-1 (380 mg, 43%) as a yellow solid.

### Synthesis of Compound 14-2.

A solution of Compound 14-2 (380 mg, 0.47 mmol, 1.0 equiv) and TFA (1 mL) in DCM (4 mL) was stirred for overnight at room temperature. The resulting mixture was concentrated under reduced pressure. The mixture was neutralized to pH 7 with NH₃ in MeOH. The residue was purified by Prep-TLC (CH₂Cl₂/MeOH=10:1) to afford Compound 14-2 (180 mg, 54%) as a yellow solid.

### Synthesis of Compound 14.

To a stirred solution of Compound 14-2 (160 mg, 0.22 mmol, 1.0 equiv) and 2-(2,6-dioxopiperidin-3-yl)-4-fluoroisoindole-1,3-dione (188 mg, 0.66 mmol, 3.0 equiv) in NMP (1.6 mL) was added DIEA (88 mg, 0.66 mmol, 3.0 equiv) at room temperature. The resulting mixture was stirred for overnight at 60° C. The reaction solution was purified by Prep-HPLC with the following conditions (Column: Xselect CSH C18 OBD Column 30*150 mm 5 μm, n; Mobile Phase A: water (0.1% FA), Mobile Phase B: ACN; Flow rate: 60 mL/min; Gradient: 28% B to 38% B in 7 min, 38% B; wave Length: 254; 220 nm; RT1 (min): 6.53) to afford Compound 14 (18 mg, 7%) as a yellow solid.

LC-MS-Compound 14: (ES, m/z): [(M−HCOOH)/2+H]⁺ 481

H-NMR-Compound 14: (400 MHz, CD₃OD, δ ppm): 1.70-1.79 (m, 3H), 1.91-1.95 (m, 7H), 2.07-2.11 (m, 1H), 2.20-2.26 (m, 1H), 2.43-2.51 (m, 2H), 2.70-2.77 (m, 2H), 2.81-2.90 (m, 3H), 3.41-3.55 (m, 6H), 3.65 (s, 4H), 4.28-4.30 (m, 1H), 4.56-4.59 (d, 4H), 5.04-5.09 (m, 1H), 6.93-7.14 (m, 4H), 7.27-7.41 (m, 6H), 7.443-7.71 (m, 5H), 8.30-8.42 (m, 1H).

### Example 13. Compound 15

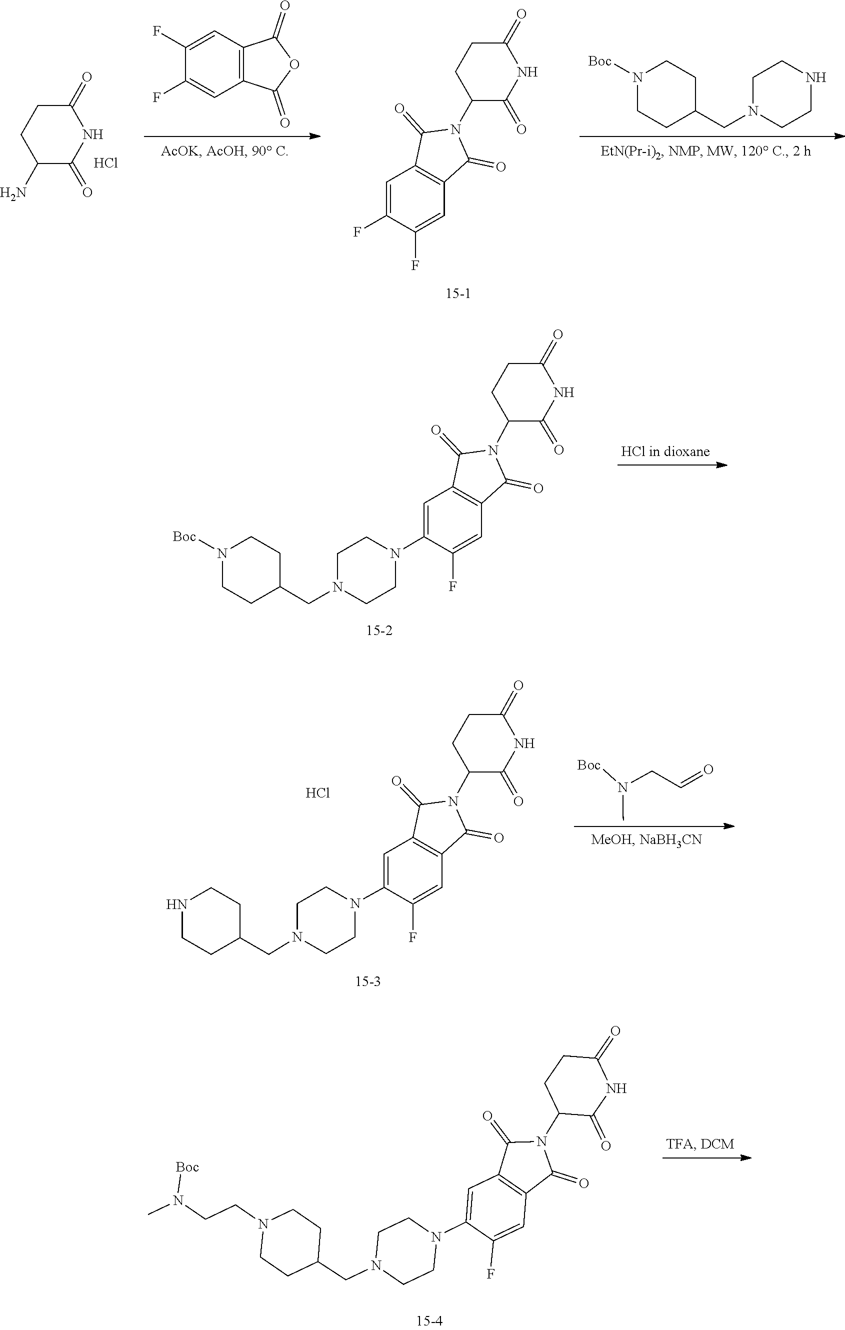

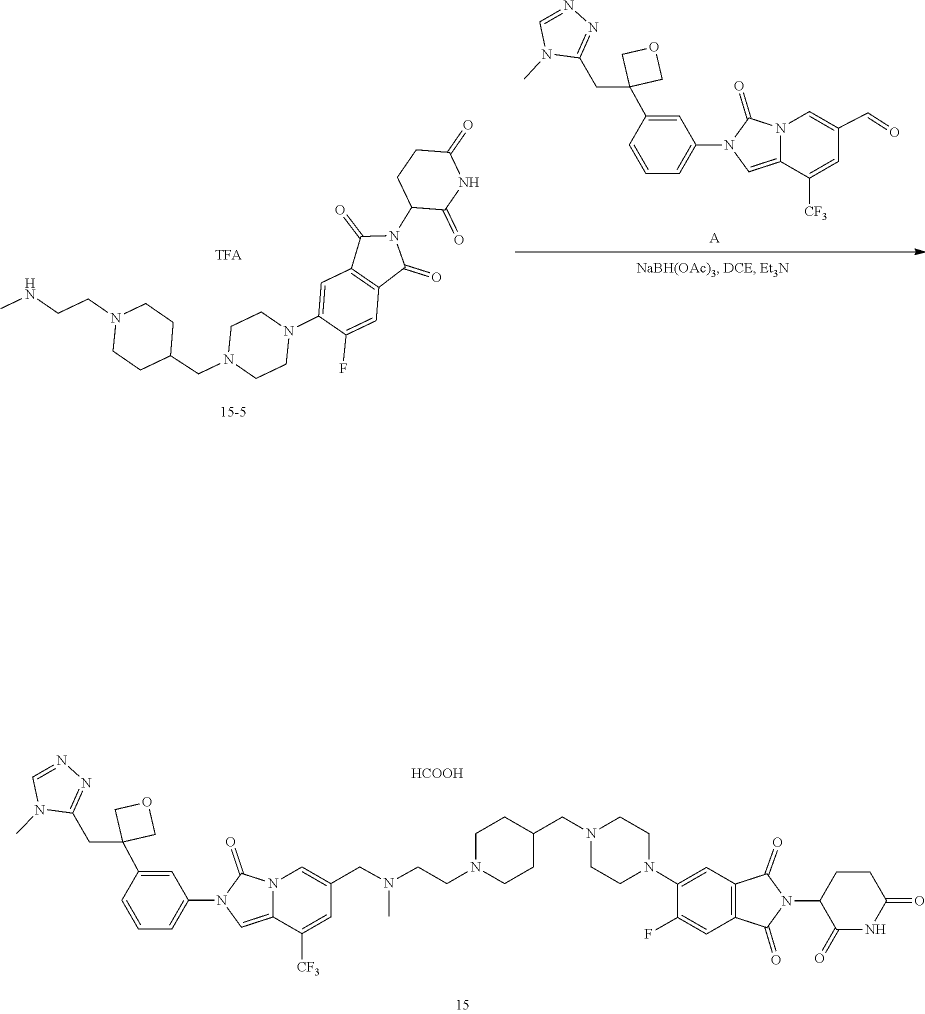

### Synthesis of Compound 15-1.

To a stirred solution of 3-aminopiperidine-2,6-dione hydrochloride (10 g, 60.757 mmol, 1 equiv) and 5,6-difluoro-2-benzofuran-1,3-dione (11.74 g, 63.795 mmol, 1.05 equiv) in AcOH (100 mL) was added AcOK (6.56 g, 66.833 mmol, 1.1 equiv) at room temperature under nitrogen atmosphere. The resulting mixture was stirred for overnight at 90° C. under nitrogen atmosphere. The resulting mixture was concentrated under reduced pressure. The resulting mixture was diluted with water (50 mL). The precipitated solids were collected by filtration and washed with water (3×10 mL). The residue was purified by trituration with MeCN (10 mL) to afford Compound 15-1 (13 g, 72.72%) as a dark green solid.

### Synthesis of Compound 15-2.

To a stirred solution of Compound 15-1 (1.8 g, 6.118 mmol, equiv) and tert-butyl 4-(piperazin-1-ylmethyl)piperidine-1-carboxylate (3.47 g, 12.236 mmol, 2 equiv) in NMP (18 mL) was added DIEA (3.95 g, 30.590 mmol, 5 equiv) dropwise at room temperature under nitrogen atmosphere. The final reaction mixture was irradiated with microwave radiation for 2 h at 120° C. The reaction solution was purified by reverse flash chromatography with the following conditions: column, C18 silica gel; mobile phase, MeCN in water (0.1% FA), 10% to 50% gradient in 40 min. This resulted in Compound 15-2 (2.2 g, 60.62%) as a dark green solid.

### Synthesis of Compound 15-3.

Into a 8 mL sealed tube were added Compound 15-2 (1.1 g, 1.973 mmol, 1 equiv) and HCl(gas) in 1,4-dioxane (11 mL) at room temperature. The resulting mixture was stirred for overnight at room temperature under nitrogen atmosphere. The resulting mixture was concentrated under reduced pressure. This resulted in Compound 15-3 (900 mg, 99.72%) as a dark green solid.

### Synthesis of Compound 15-4.

To a stirred solution of Compound 15-3 (400 mg, 0.874 mmol, 1 equiv) and tert-butyl N-methyl-N-(2-oxoethyl)carbamate (227.16 mg, 1.311 mmol, 1.5 equiv) in MeOH (10 mL) were added NaBH₃CN (109.88 mg, 1.748 mmol, 2 equiv). The resulting mixture was stirred for overnight at room temperature. The reaction was quenched by the addition of sat. NH₄Cl (aq.) (40 mL) at room temperature. The resulting mixture was extracted with CH₂Cl₂ (2×50 mL). The combined organic layers were concentrated under reduced pressure. The residue was purified by reverse flash chromatography with the following conditions: column, C18 silica gel; mobile phase, MeCN in Water (0.1% HOAc), 5% to 60% gradient in 30 min; detector, UV 220 nm. This resulted in Compound 15-4 (310 mg, 57.68%) as a light yellow solid.

### Synthesis of Compound 15-5.

To a stirred solution of Compound 15-4 (310 mg, 0.504 mmol, 1 equiv) in DCM (4 mL) were added TFA (1 mL). The resulting mixture was stirred for 4 h at room temperature. The resulting mixture was concentrated under reduced pressure. This resulted in Compound 15-5 (330 mg, crude) as a light yellow solid.

### Synthesis of Compound 15.

To a stirred solution of Compound 15-5 (170 mg, 0.330 mmol, 1 equiv) and Intermediate A (151.11 mg, 0.330 mmol, 1 equiv) in DCE (15 mL) were added TEA (33.43 mg, 0.330 mmol, 1 equiv) and STAB (140.03 mg, 0.660 mmol, 2 equiv). The resulting mixture was stirred for overnight at room temperature. The resulting mixture was diluted with H2O/AcOH=10/1 (20 mL). The resulting mixture was extracted with CH2Cl2/MeOH=10/1 (2×20 mL). The combined organic layers were concentrated under reduced pressure. The residue was purified by Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 10% B to 20% B in 7 min, 20% B; Wave Length: 254; 220 nm; RT1 (min): 6.92) to afford Compound 15 (48.7 mg, 14.71%) as a yellow solid.

LCMS-Compound 15: (ES, m/z): [M−HCOOH+H]⁺ 956

NMR-Compound 15: (400 MHz, CD₃OD, δ ppm): δ1.54-1.65 (m, 2H), 2.08-2.15 (m, 4H), 2.41 (s, 3H), 2.72-2.88 (m, 9H), 2.99 (s, 3H), 3.01-3.15 (m, 6H), 3.42-3.50 (m, 6H), 3.56-3.68 (m, 5H), 5.06-5.15 (m, 5H), 6.98-7.00 (d, 1H), 7.10 (s, 1H), 7.16 (s, 1H), 7.27 (s, 1H), 7.52-7.61 (m, 2H), 7.63-7.64 (m, 2H), 7.76 (s, 1H), 8.21 (s, 1H).

### Example 14. Compound 16

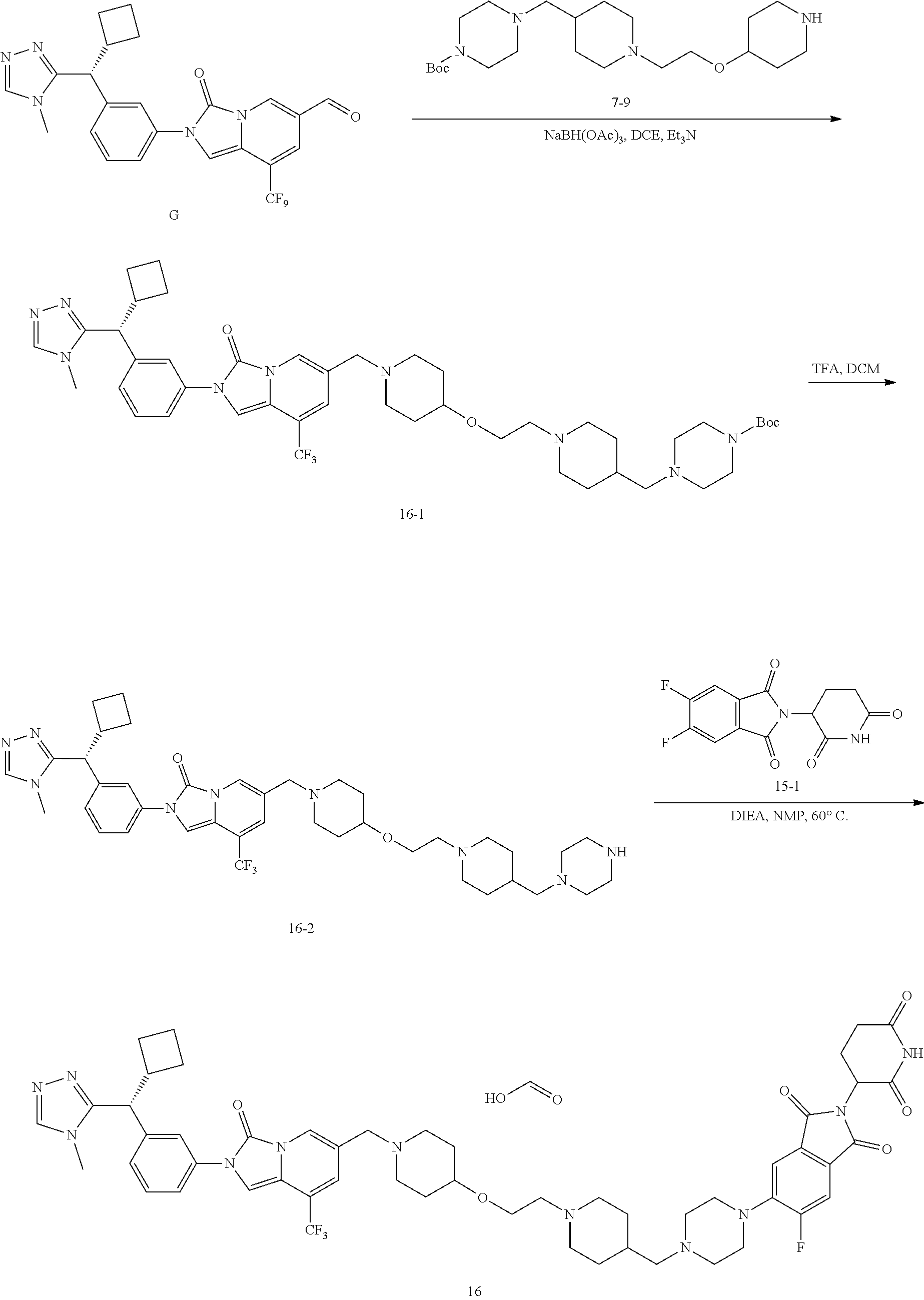

### Synthesis of Compound 16-1.

To a stirred solution of Intermediate G (400 mg, 0.878 mmol, 1 equiv) and Compound 7-9 (540.93 mg, 1.317 mmol, 1.5 equiv) in DCE (10 mL) was added Et₃N (88.87 mg, 0.878 mmol, 1 equiv) and NaBH(OAc)₃ (372.28 mg, 1.756 mmol, 2 equiv). The resulting mixture was stirred for overnight at room temperature. The reaction was quenched by the addition of sat. NaHCO₃ (aq.) (10 mL). The resulting mixture was extracted with CH₂Cl₂/MeOH=10/1 (2×15 mL). The combined organic layers were concentrated under reduced pressure. The residue was purified by reverse flash chromatography with the following conditions: column, C18 silica gel; mobile phase, MeCN in water (10 mmoL/L NH₄HCO₃), 20% to 80% gradient in 35 min; detector, UV 254 nm. This resulted in Compound 16-1 (305 mg, 40.85%) as a yellow solid.

### Synthesis of Compound 16-2.

To a stirred solution of Compound 16-1 (0.305 mg, 0.359 mmol, 1 equiv) in DCM (4 mL) was added TFA (1 mL). The resulting mixture was stirred for 2 h at room temperature. The resulting mixture was concentrated under reduced pressure. The residue was purified by Prep-TLC (CH₂Cl₂/MeOH 10:1) to afford Compound 16-2 (200 mg, 74.33%) as a yellow solid.

### Synthesis of Compound 16.

To a stirred solution of Compound 16-2 (200 mg, 0.267 mmol, 1 equiv) and Compound 15-1 (235.39 mg, 0.801 mmol, 3 equiv) in NMP (2 mL) was added DIEA (68.94 mg, 0.534 mmol, 2 equiv). The resulting mixture was stirred for overnight at 60° C. The crude product was purified by Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 10% B to 20% B in 7 min, 20% B; Wave Length: 254; 220 nm; RT1 (min): 6.20) to afford Compound 16 (40.4 mg, 14.16%) as a yellow solid.

LCMS-Compound 16: (ES, m/z): [M−HCOOH+H]+ 1024

NMR-Compound 16: (400 MHz, CD₃OD, δ ppm): δ1.42-1.77 (m, 5H), 1.82-2.15 (m, 11H), 2.20-2.55 (m, 6H), 2.64-2.76 (m, 7H), 2.80-3.15 (m, 5H), 3.35 (s, 3H), 3.53-3.63 (m, 9H), 3.82-3.84 (m, 2H), 4.29-4.31 (d, 1H), 5.09-5.13 (m, 1H), 7.12-7.16 (d, 2H), 7.29-7.32 (m, 2H), 7.48-7.52 (m, 2H), 7.55-7.58 (m, 1H), 7.64-7.65 (m, 2H), 7.74 (s, 1H), 8.22-8.31 (m, 3H).

### Example 15. Compound 17

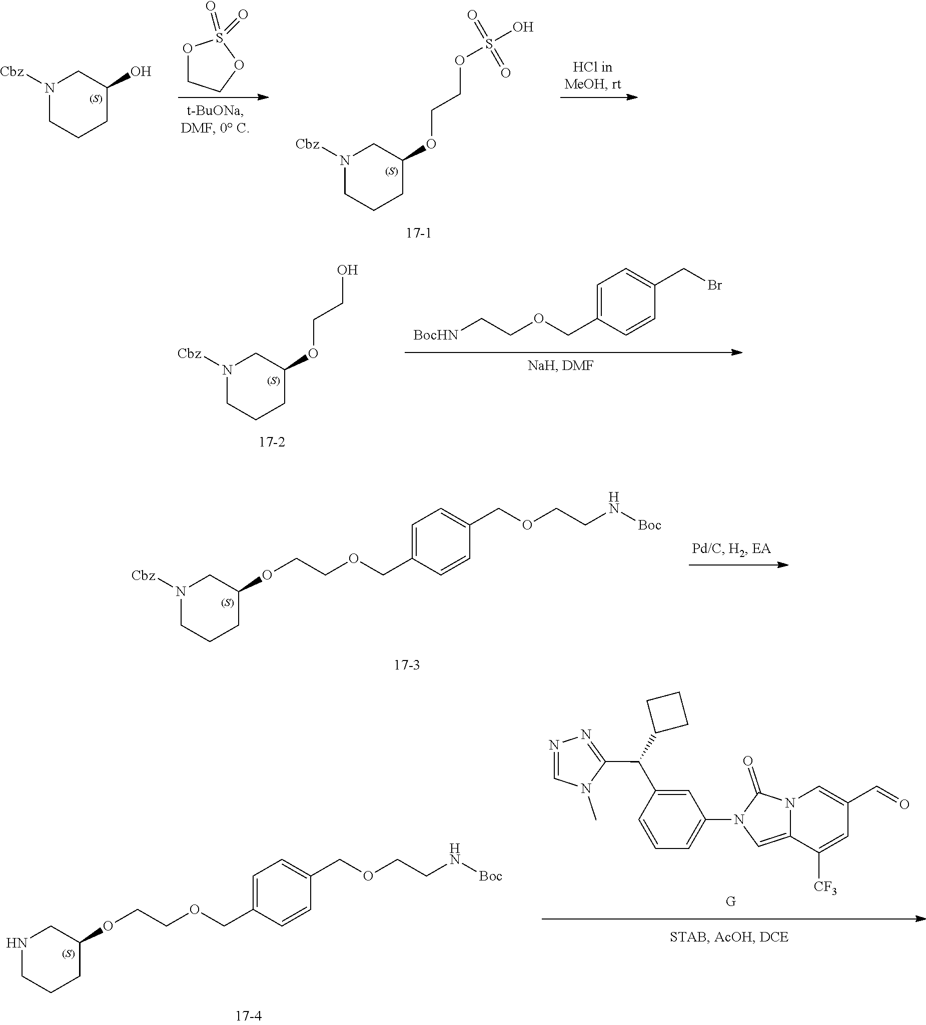

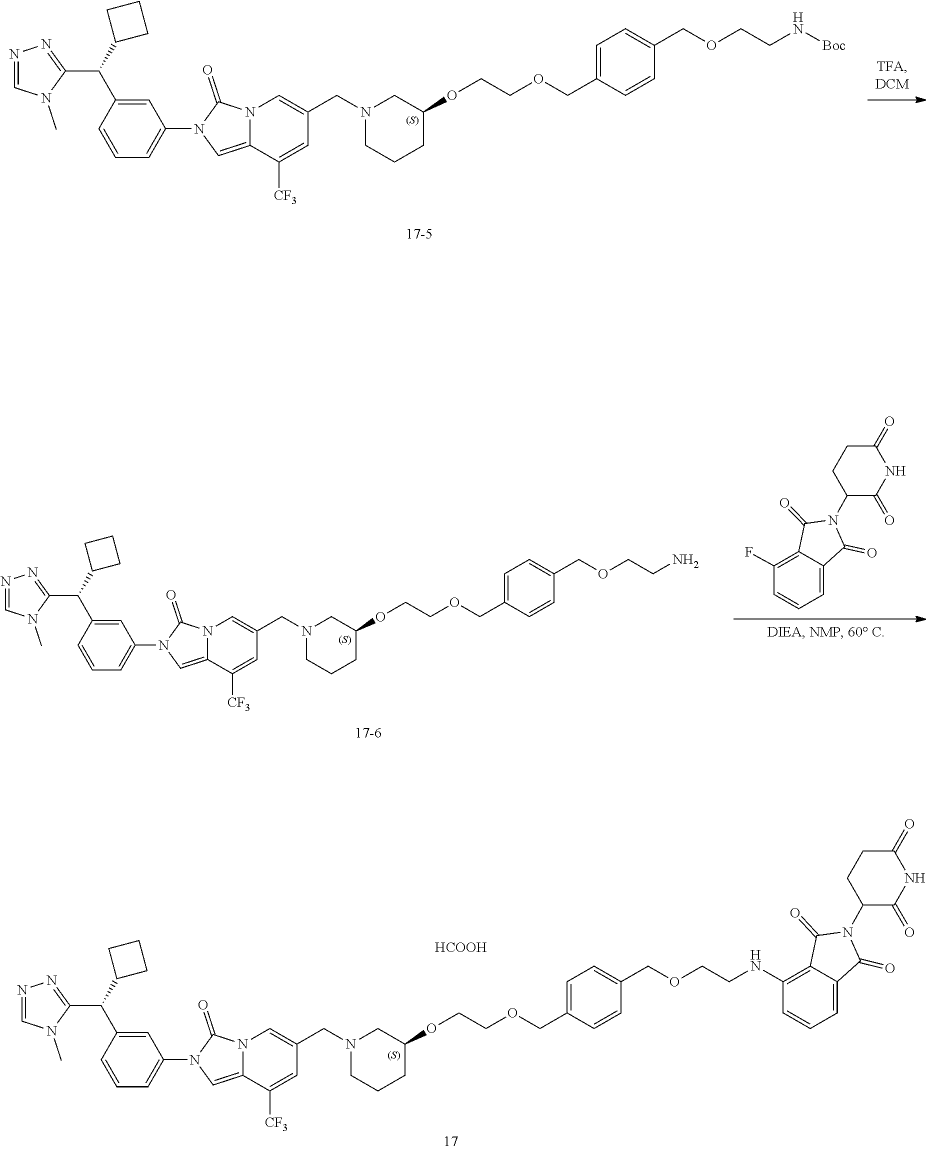

### Synthesis of Compound 17-1.

To a stirred solution of benzyl (3S)-3-hydroxypiperidine-1-carboxylate (10 g, 42.502 mmol, 1 equiv) and 1,3,2lambda6-dioxathiolane-2,2-dione (18.46 g, 148.757 mmol, 3.5 equiv) in DMF (100 mL) was added t-BuONa (14.30 g, 148.757 mmol, 3.5 equiv) in portions at −20° C. The resulting mixture was stirred for overnight at room temperature. The residue was dissolved in water (1000 mL). The aqueous layer was extracted with EtOAc (3×500 mL). The residue was acidified to pH 6 with conc. HCl. The resulting mixture was extracted with EtOAc (3×800 mL). The combined organic layers were washed with water (3×500 mL), dried over anhydrous Na2SO4. After filtration, the filtrate was concentrated under reduced pressure. This resulted in Compound 17-1 (15 g, 88.38%) as a brown oil.

### Synthesis of Compound 17-2.

A solution of Compound 17-1 (15 g, 41.737 mmol, 1 equiv) and acetyl chloride (22 mL) in methanol (150 mL) was stirred for 3 h at room temperature. The mixture was neutralized to pH 7 with saturated NaHCO₃ (aq.). The resulting mixture was extracted with EtOAc (3×200 mL). The combined organic layers were washed with water (3×100 mL), dried over anhydrous Na2SO4. After filtration, the filtrate was concentrated under reduced pressure. The residue was purified by silica gel column chromatography, eluted with PE/EA (1:1) to afford Compound 17-2 (5.4 g, 41.69%) as a colorless oil.

### Synthesis of Compound 17-3.

To a solution of Compound 17-2 (15 g, 80.535 mmol, 1.00 equiv) in THF was added sodium hydride (60% in oil, 3.87 g) at 0 degrees C. The mixture was stirred for 15 min. Tert-butyl (2-((4-(bromomethyl)benzyl)oxy)ethyl)carbamate (31.89 g, 120.802 mmol, 1.5 equiv) was added and the mixture was allowed to warm to room temperature and stirred for overnight. The reaction mixture was quenched by water and extracted with DCM (3*250 mL). The residue was purified by silica gel column chromatography, eluted with PE/EA (5:1) to afford Compound 17-3 (6 g, 18.76%) as a white oil.

### Synthesis of Compound 17-4.

To a solution of Compound 17-3 (1.8 g, 3.317 mmol, 1.00 equiv) in EtOAc (18 mL) was added Pd/C (10%, 0.18 g) under nitrogen atmosphere in a 25 mL round-bottom flask. The mixture was hydrogenated at room temperature for overnight under hydrogen atmosphere using a hydrogen balloon, filtered through a Celite pad and concentrated under reduced pressure. This resulted in Compound 17-4 (1.2 g, 80.59%) as a colorless oil.

### Synthesis of Compound 17-5.

To a stirred solution of 17-4 (493.36 mg, 1.208 mmol, 1.1 equiv) and Intermediate G (500 mg, 1.098 mmol, 1.00 equiv) in DCE (10 mL) were added Et₃N (111.09 mg, 1.098 mmol, 1 equiv) and STAB (465.35 mg, 2.196 mmol, 2 equiv). The resulting mixture was stirred for overnight at room temperature. The reaction was quenched by the addition of Water (20 mL). The resulting mixture was extracted with CH₂Cl₂/MeOH=10/1 (2×20 mL). The combined organic layers were concentrated under reduced pressure. The residue was purified by reverse flash chromatography with the following conditions: column, C18 silica gel; mobile phase, MeCN in Water (10 mmol/L NH₄HCO₃), 25% to 85% gradient in 30 min; detector, UV 254 nm. This resulted in Compound 17-5 (420 mg, 45.12%) as a yellow solid.

### Synthesis of Compound 17-6.

To a stirred solution of Compound 17-5 (0.420 mg, 0.495 mmol, 1 equiv) in DCM (4 mL) was added TFA (1 mL). The resulting mixture was stirred for 4 h at room temperature. The resulting mixture was concentrated under reduced pressure. The residue was purified by Prep-TLC (CH₂Cl₂/MeOH 10:1) to afford Compound 17-6 (280 mg, 75.59%) as a yellow solid.

### Synthesis of Compound 17.

To a stirred solution of Compound 17-6 (150 mg, 0.201 mmol, 1 equiv) and 2-(2,6-dioxopiperidin-3-yl)-4-fluoroisoindole-1,3-dione (166.21 mg, 0.603 mmol, 3 equiv) in NMP (1.5 mL) was added DIEA (51.85 mg, 0.402 mmol, 2 equiv). The resulting mixture was stirred for overnight at room temperature. The reaction solution was purified by Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 29% B to 40% B in 7 min, 40% B; Wave Length: 254; 220 nm; RT1 (min): 6.12) to afford Compound 17 (50 mg, 23.74%) as a yellow solid.

LCMS-Compound 17: (ES, m/z): [M−HCOOH+H]⁺ 1004

NMR-Compound 17: (400 MHz, CD₃OD, δ ppm): δ1.32-1.92 (m, 8H), 2.01-2.44 (m, 4H), 2.70-2.93 (m, 5H), 3.29-3.32 (m, 2H), 3.47-3.70 (m, 14H), 4.27-4.30 (d, 1H), 4.51-4.53 (d, 4H), 5.06-5.10 (m, 1H), 7.00-7.02 (m, 2H), 7.08-7.11 (m, 2H), 7.27-7.32 (m, 5H), 7.46-7.50 (m, 2H), 7.61-7.65 (m, 2H), 7.71 (s, 1H), 8.35 (s, 1H), 8.41 (s, 1H).

### Example 16. Compound 18

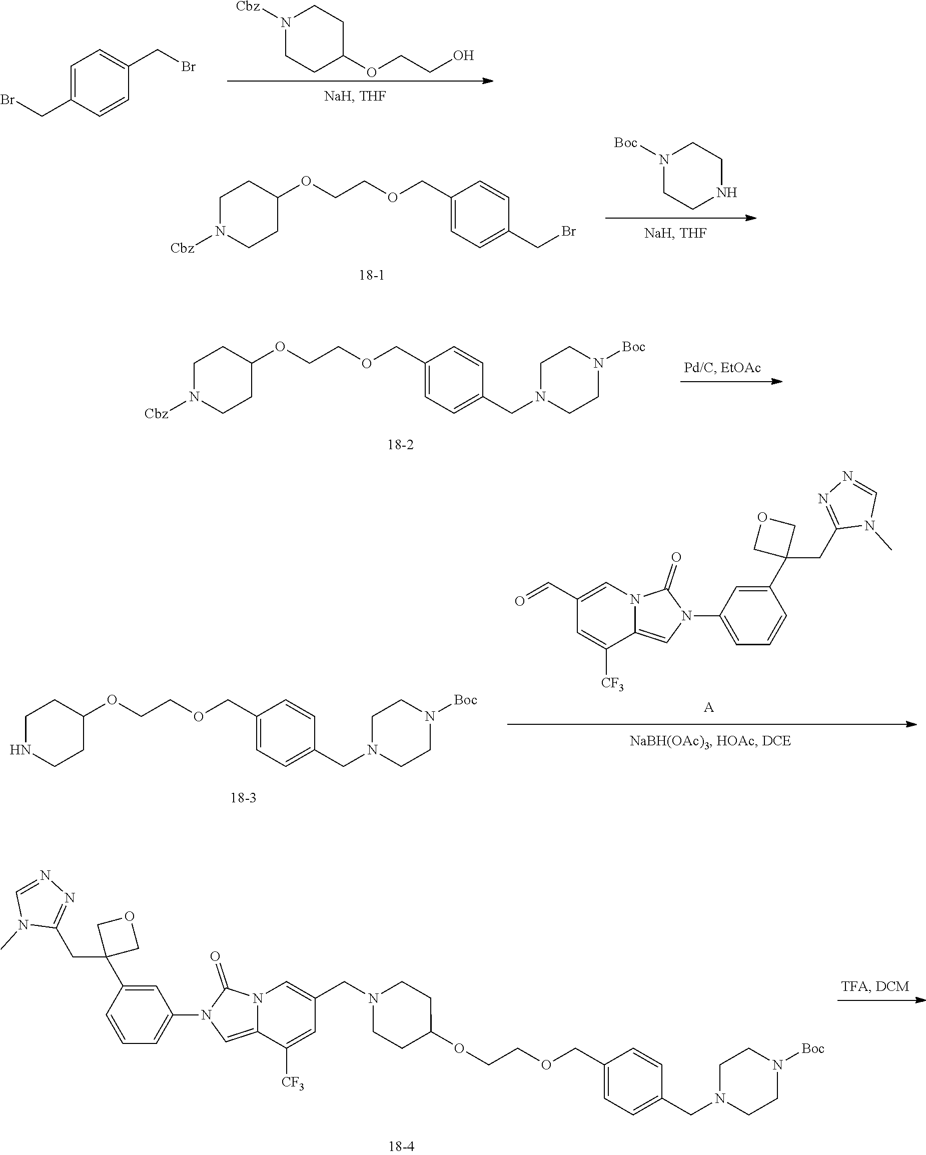

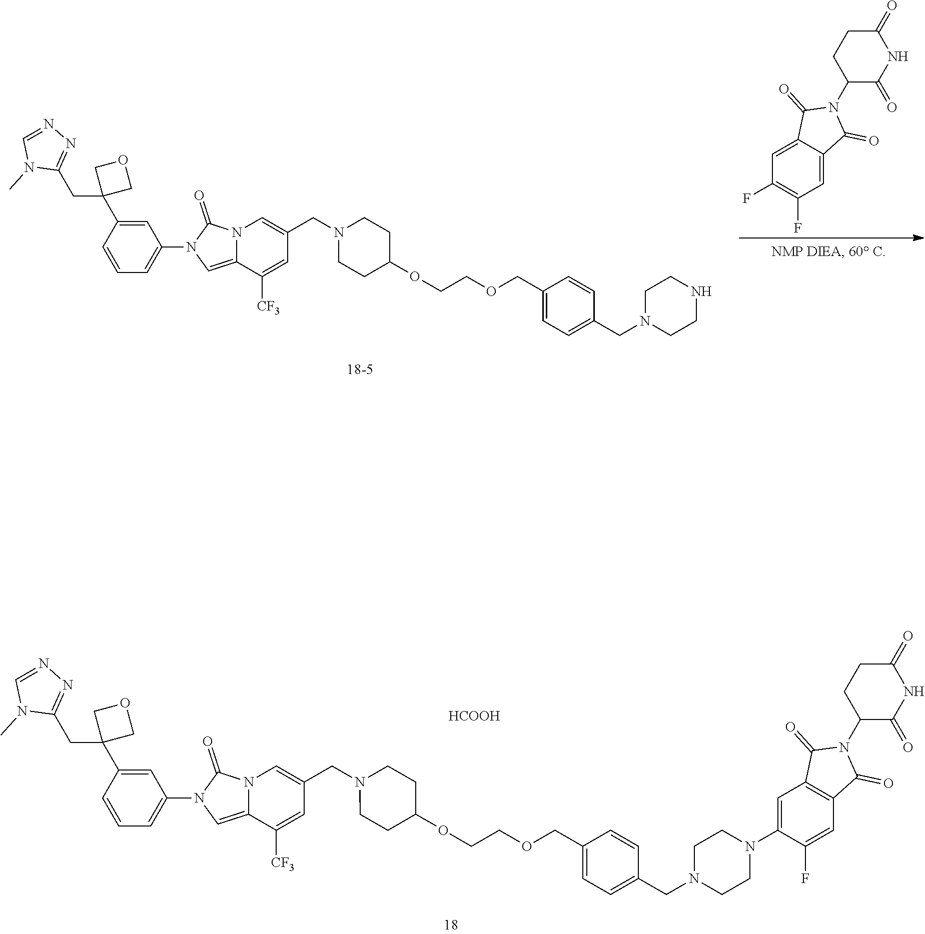 [SMILES: Cn1cnnc1CC1(c2cccc(-n3cc4c(C(F)(F)F)cc(CN5CCC(OCCOCc6ccc(CN7CCNCC7)cc6)CC5)cn4c3=O)c2)COC1 ; O=C1CCC(N2C(=O)c3cc(F)c(F)cc3C2=O)C(=O)N1 ; Cn1cnnc1CC1(c2cccc(-n3cc4c(C(F)(F)F)cc(CN5CCC(OCCOCc6ccc(CN7CCN(c8cc9c(cc8F)C(=O)N(C8CCC(=O)NC8=O)C9=O)CC7)cc6)CC5)cn4c3=O)c2)COC1 ; "HCOOH"]

### Synthesis of Compound 18-1.

To a solution of benzyl 4-(2-hydroxyethoxy) piperidine-1-carboxylate (14 g, 51.14 mmol, 1.5 equiv) in THF (90 mL) was added sodium hydride (60% in oil, 1.6 g) at 0 degrees C. The mixture was stirred for 30 min. 1,4-bis(bromomethyl)-benzene (9 g, 34.09 mmol, 1.0 equiv) was added and the mixture was allowed to warm to room temperature and stirred for overnight. The reaction mixture was quenched by water (200 mL) and extracted with DCM (3×250 mL). The residue was purified by silica gel column chromatography, eluted with PE/EA (1:1) to afford Compound 18-1 (8 g, 47%) as a colorless oil.

### Synthesis of Compound 18-2.

To a solution of tert-butyl piperazine-1-carboxylate (4.8 g, 25.95 mmol, 1.5 equiv) in THF (80 mL) was added sodium hydride (60% in oil, 0.83 g) at 0 degrees C. The mixture was stirred for 30 mini. Compound 18-1 (8 g, 17.30 mmol, 1.0 equiv) was added and the mixture was allowed to warm to room temperature and stirred for overnight. The reaction mixture was quenched by water (200 mL) and extracted with DCM (3×100 mL). The residue was purified by silica gel column chromatography, eluted with PE/EA (1:1) to afford Compound 18-2 (6 g, 61%) as a colorless oil.

### Synthesis of Compound 18-3.

To a solution of Compound 18-2 (6 g, 10.58 mmol, 1.0 equiv) in 180 mL MeOH was added Pd/C (10%, 1.2 g) under nitrogen atmosphere in a 250 mL round-bottom flask. The mixture was hydrogenated at room temperature for 2 h under hydrogen atmosphere using a hydrogen balloon, filtered through a Celite pad and concentrated under reduced pressure. This resulted in Compound 18-3 (4.5 g, 98%) as a colorless oil.

### Synthesis of Compound 18-4.

To a stirred solution of Intermediate A (1 g, 2.18 mmol, 1.0 equiv) and Compound 18-3 (1.4 g, 3.27 mmol, 1.5 equiv) in DCE (10 mL) were added TEA (0.22 g, 2.18 mmol, 1.0 equiv) and NaBH(OAc)₃ (0.93 g, 4.37 mmol, 2.0 equiv) at room temperature. The resulting mixture was stirred for overnight at room temperature. The reaction was quenched with water at room temperature. The resulting mixture was extracted with CH₂Cl₂ (3×20 mL). The combined organic layers were washed with water (3×10 mL), dried over anhydrous Na₂SO₄. After filtration, the filtrate was concentrated under reduced pressure. The residue was purified by Prep-TLC (CH₂Cl₂/MeOH=10:1) to afford Compound 18-4 (900 mg, 47%) as a yellow solid.

### Synthesis of Compound 18-5.

A solution of Compound 18-4 (900 mg, 1.02 mmol, 1.0 equiv) and TFA (2 mL) in DCM (8 mL) was stirred for overnight at room temperature. The resulting mixture was concentrated under reduced pressure. The residue was purified by Prep-TLC (CH₂Cl₂/MeOH=10:1) to afford Compound 18-5 (650 mg, 82%) as a yellow oil.

### Synthesis of Compound 18.

To a stirred solution of Compound 18-5 (250 mg, 0.32 mmol, 1.0 equiv) and 2-(2,6-dioxopiperidin-3-yl)-5,6-difluoroisoindole-1,3-dione (284 mg, 0.96 mmol, 3.0 equiv) in NMP (2.5 mL) was added DIEA (125 mg, 0.96 mmol, 3.0 equiv) at room temperature. The resulting mixture was stirred for overnight at 60° C. The reaction solution was purified by Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 12% B to 23% B in 8 min, 23% B; Wave Length: 254; 220 nm; RT1 (min): 7.22) to afford Compound 18 (123 mg, 34%) as a yellow solid.

LC-MS-Compound 18: (ES, m/z): [M-COOH]⁺ 1049

H-NMR-Compound 18: (400 MHz, CDCl₃, δ ppm): 1.76 (s, 2H), 2.00-2.09 (m, 2H), 2.13-2.17 (m, 1H), 2.45 (s, 2H), 2.69-2.87 (m, 4H), 2.90-2.93 (m, 8H), 3.32 (s, 4H), 3.41 (s, 2H), 3.50-3.54 (m, 1H), 3.60-3.64 (m, 8H), 4.59 (s, 2H), 4.92-4.96 (m, 1H), 5.06-5.08 (d, 2H), 5.14-5.15 (d, 2H), 6.72 (s, 1H), 6.77-6.79 (m, 1H), 7.09 (s, 1H), 7.27-7.28 (d, 1H), 7.35-7.40 (m, 6H), 7.44-7.49 (m, 2H), 7.63 (s, 1H), 7.90 (s, 1H), 8.29 (s, 1H).

### Example 17. Compound 19

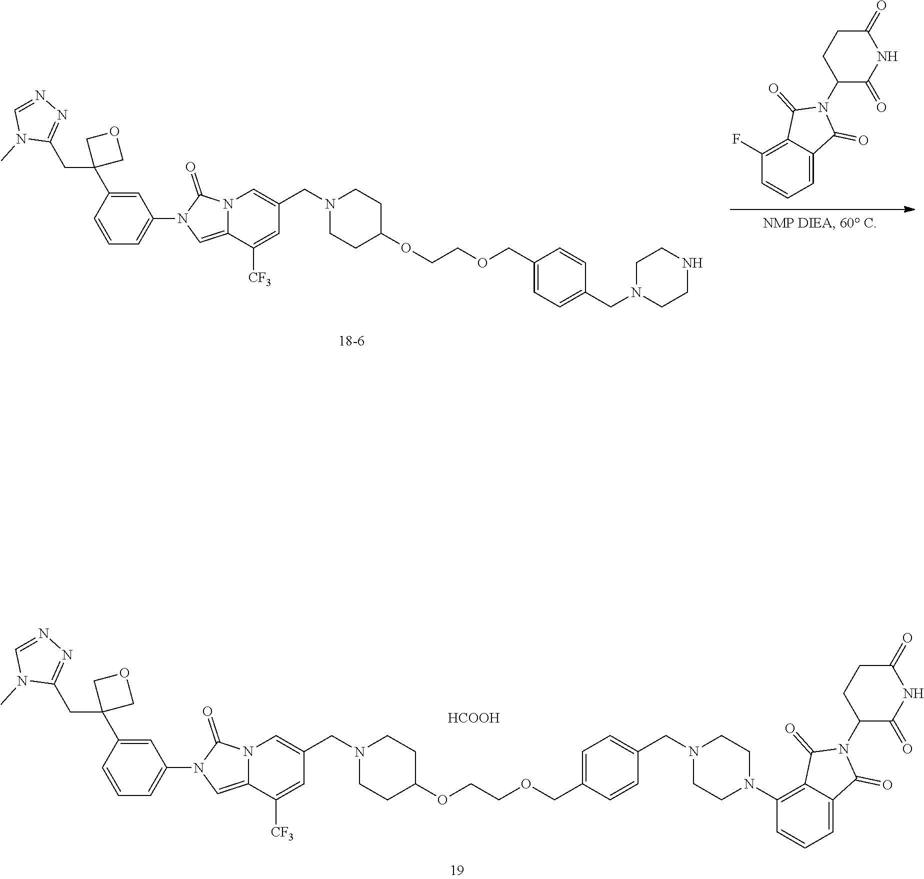 [SMILES: Cn1cnnc1CC1(c2cccc(-n3cc4c(C(F)(F)F)cc(CN5CCC(OCCOCc6ccc(CN7CCNCC7)cc6)CC5)cn4c3=O)c2)COC1 ; O=C1CCC(N2C(=O)c3cccc(F)c3C2=O)C(=O)N1 ; Cn1cnnc1CC1(c2cccc(-n3cc4c(C(F)(F)F)cc(CN5CCC(OCCOCc6ccc(CN7CCN(c8cccc9c8C(=O)N(C8CCC(=O)NC8=O)C9=O)CC7)cc6)CC5)cn4c3=O)c2)COC1 ; "HCOOH"]

### Synthesis of Compound 19.

To a stirred solution of Compound 18-6 (350 mg, 0.45 mmol, 1.0 equiv) and 2-(2,6-dioxopiperidin-3-yl)-4-fluoroisoindole-1,3-dione (374 mg, 1.35 mmol, 3.0 equiv) in NMP (3.5 mL) was added DIEA (175 mg, 1.36 mmol, 3.0 equiv) at room temperature. The resulting mixture was stirred for overnight at 60° C. The reaction solution was purified by Prep-HPLC with the following conditions (Column: Xselect CSH C18 OBD Column 30*150 mm 5 μm, n; Mobile Phase A: water (0.1% FA), Mobile Phase B: ACN; Flow rate: 60 mL/min; Gradient: 11% B to 23% B in 7 min, 23% B; Wave Length: 254; 220 nm; RT1 (min): 6.18) to afford Compound 19 (113.9 mg, 22.81%) as a yellow solid.

LC-MS-Compound 19: (ES, m/z): [M-COOH]⁺ 1031

H-NMR-Compound 19: (400 MHz, CD₃OD, δ ppm): 1.71-1.77 (m, 2H), 1.83-1.88 (m, 2H), 1.95-1.97 (m, 1H), 2.70-2.88 (m, 4H), 2.95-2.97 (m, 8H), 3.06-3.08 (m, 2H), 3.44 (s, 4H), 3.61-3.72 (m, 9H), 3.91 (s, 2H), 4.60 (s, 2H), 5.06-5.12 (m, 5H), 6.93-6.95 (d, 1H), 7.11-7.16 (m, 2H), 7.30-7.33 (m, 2H), 7.39-7.49 (m, 6H), 7.61-7.69 (m, 2H), 7.80 (s, 1H), 8.20 (s, 1H), 8.31 (s, 1H).

### Example 18. Compound 20

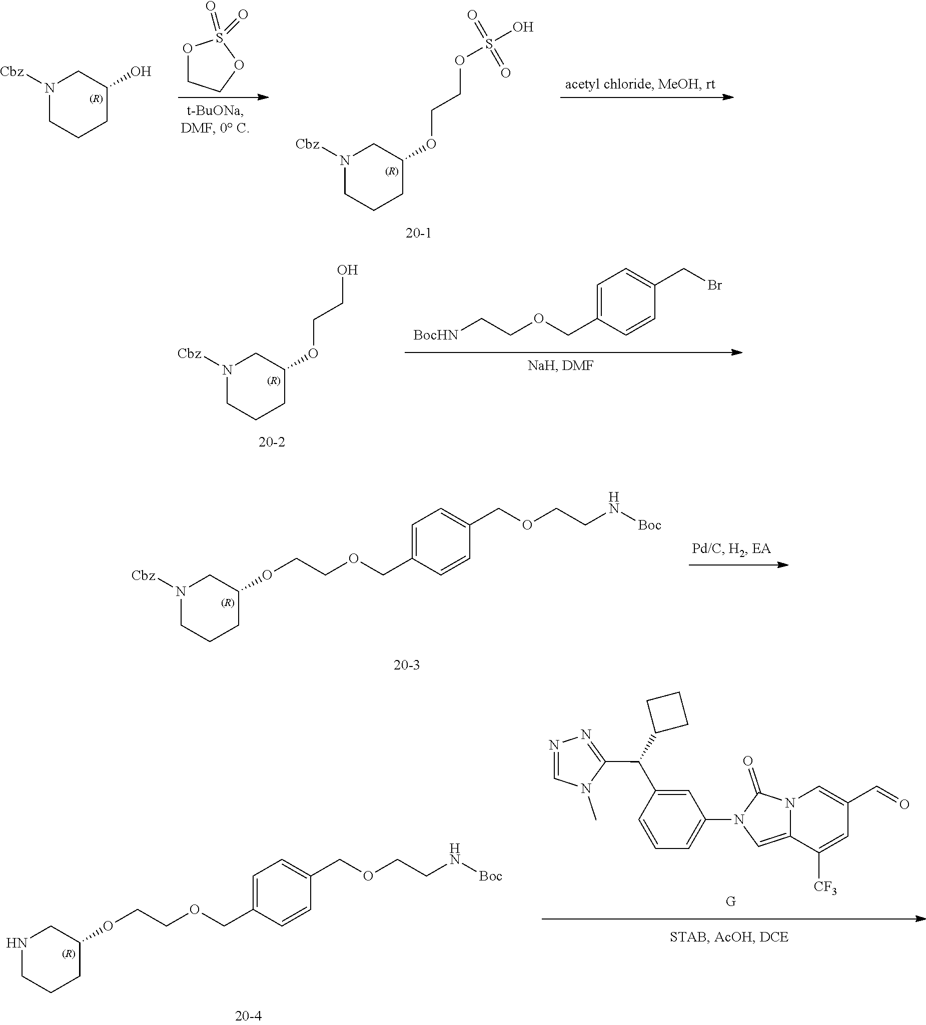

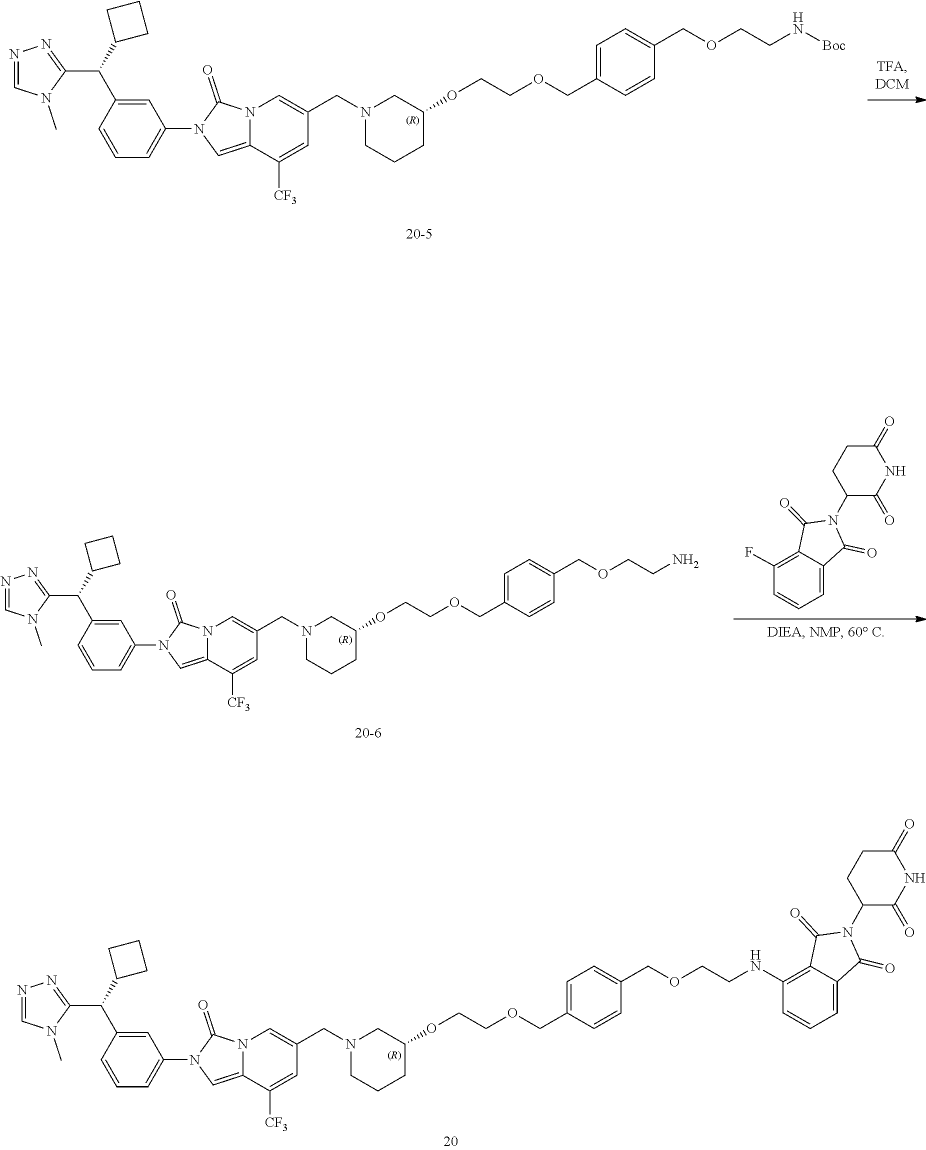

### Synthesis of Compound 20-1.

Into a 250 mL 3-necked round-bottom flask were added benzyl (3R)-3-hydroxypiperidine-1-carboxylate (10 g, 42.50 mmol, 1.0 equiv), DMF (100 mL), 1,3,2lambda6-dioxathiolane-2,2-dione (18.5 g, 148.75 mmol, 3.5 equiv) and t-BuONa (14.3 g, 148.75 mmol, 3.5 equiv) at 0° C. The resulting mixture was stirred for overnight at room temperature. The reaction was quenched by the addition of water (200 mL) at room temperature. The aqueous layer was extracted with EtOAc (3×100 mL). The resulting mixture was concentrated under reduced pressure. The crude product was used in the next step directly without further purification. This resulted in Compound 20-1 (20 g, 95%) as an off-white oil.

### Synthesis of Compound 20-2.

Into a 500 mL 3-necked round-bottom flask were added Compound 20-1 (20 g, 55.650 mmol, 1 equiv), MeOH (253 mL) and acetyl chloride (36 mL) at 0° C. The resulting mixture was stirred for 3 h at room temperature. The reaction was quenched with 5% NaHCO₃ (500 mL) at room temperature. The aqueous layer was extracted with methyl tert-buty ether (3×100 mL). The residue was purified by silica gel column chromatography, eluted with PE/EA (1:1) to afford benzyl Compound 20-2 (6 g, 39%) as an off-white oil.

### Synthesis of Compound 20-3.

To a solution of benzyl Compound 20-2 (6 g, 21.48 mmol, 1.5 equiv) in 60 ml DMF was added NaH (1.15 g, 17.184 mmol, 1.2 equiv, 60%) at 0° C. The mixture was stirred for 60 min. tert-butyl N-(2-{[4-(bromomethyl) phenyl] methoxy} ethyl) carbamate (4.9 g, 14.32 mmol, 1.0 equiv) was added and the mixture was allowed to warm to room temperature and stirred for 4 h. The reaction mixture was quenched by water and extracted with EA (3×25 mL). The residue was purified by silica gel column chromatography, eluted with PE/EA (5:1) to afford Compound 20-3 (3 g, 39%) as an off-white oil.

### Synthesis of Compound 20-4.

To a solution of Compound 20-3 (3 g, 5.52 mmol, 1.0 equiv) in 90 mL EA was added Pd/C (20%, 0.6 g) in a pressure tank. The mixture was hydrogenated at room temperature under hydrogen for 4 h, filtered through a Celite pad and concentrated under reduced pressure. This resulted in Compound 20-4 (2 g, 89%) as an off-white oil.

### Synthesis of Compound 20-5.

To a stirred solution of Intermediate G (700 mg, 1.53 mmol, 1.0 equiv) and Compound 20-4 (628 mg, 1.53 mmol, 1.0 equiv) in DCE (7 mL) was added TEA (155 mg, 1.53 mmol, 1.0 equiv) at room temperature. The resulting mixture was stirred for 30 min at room temperature. To the above mixture was added STAB (651 mg, 3.07 mmol, 2.0 equiv). The resulting mixture was stirred for additional overnight at room temperature. The reaction was quenched by the addition of water (30 mL) at room temperature. The aqueous layer was extracted with CH₂Cl₂ (3×20 mL). The resulting mixture was concentrated under reduced pressure. The residue was purified by Prep-TLC (CH₂Cl₂/MeOH=10:1) to afford Compound 20-5 (400 mg, 31%) as a yellow solid.

### Synthesis of Compound 20-6.

A solution of Compound 20-5 (380 mg, 0.44 mmol, 1.0 equiv) and TFA (1 mL) in DCM (3 mL) was stirred for overnight at room temperature. The resulting mixture was concentrated under reduced pressure. The residue was neutralized to pH 7 with NH₃ in MeOH. The residue was purified by Prep-TLC (CH₂Cl₂/MeOH=10:1) to afford Compound 20-6 (300 mg, 90%) as a yellow solid.

### Synthesis of Compound 20.

To a stirred solution of Compound 20-6 (260 mg, 0.34 mmol, 1.0 equiv) and 2-(2,6-dioxopiperidin-3-yl)-4-fluoroisoindole-1,3-dione (288 mg, 1.04 mmol, 3.0 equiv) in NMP was added DIEA (134 mg, 1.04 mmol, 3.0 equiv) at room temperature. The resulting mixture was stirred for overnight at 60° C. The reaction solution was purified by Prep-HPLC with the following conditions (Column: Xselect CSH (C18 OBD Column 30*150 mm 5 μm, n; Mobile Phase A: water (0.1% FA), Mobile Phase B: ACN; Flow rate: 60 mL/min; Gradient: 27% B to 38% B in 9 min, 38% B; Wave Length: 254; 220 nm; RT1 (min): 8.85) to afford Compound 20 (19 mg, 5%) as a yellow solid.

LC-MS-Compound 20: (ES, m/z): [M+H]⁺ 1004

H-NMR-Compound 20: (400 MHz, CD₃OD, δ ppm): 1.31-1.34 (m, 2H), 1.59-1.60 (m, 1H), 1.78-1.81 (m, 2H), 1.90-1.92 (m, 6H), 2.04-2.31 (m, 4H), 2.78-2.91 (m, 4H), 3.39 (s, 2H), 3.47-3.49 (m, 3H), 3.54-3.61 (m, 3H), 3.65-3.67 (m, 2H), 3.68-3.70 (m, 4H), 4.27-4.29 (d, 1H), 4.52 (s, 4H), 5.05-5.08 (m, 1H), 7.00-7.02 (d, 1H), 7.09 (s, 2H), 7.25-7.31 (m, 6H), 7.46-7.50 (t, 2H), 7.61-7.68 (m, 3H), 8.31 (s, 1H).

### Example 19. Compound 21

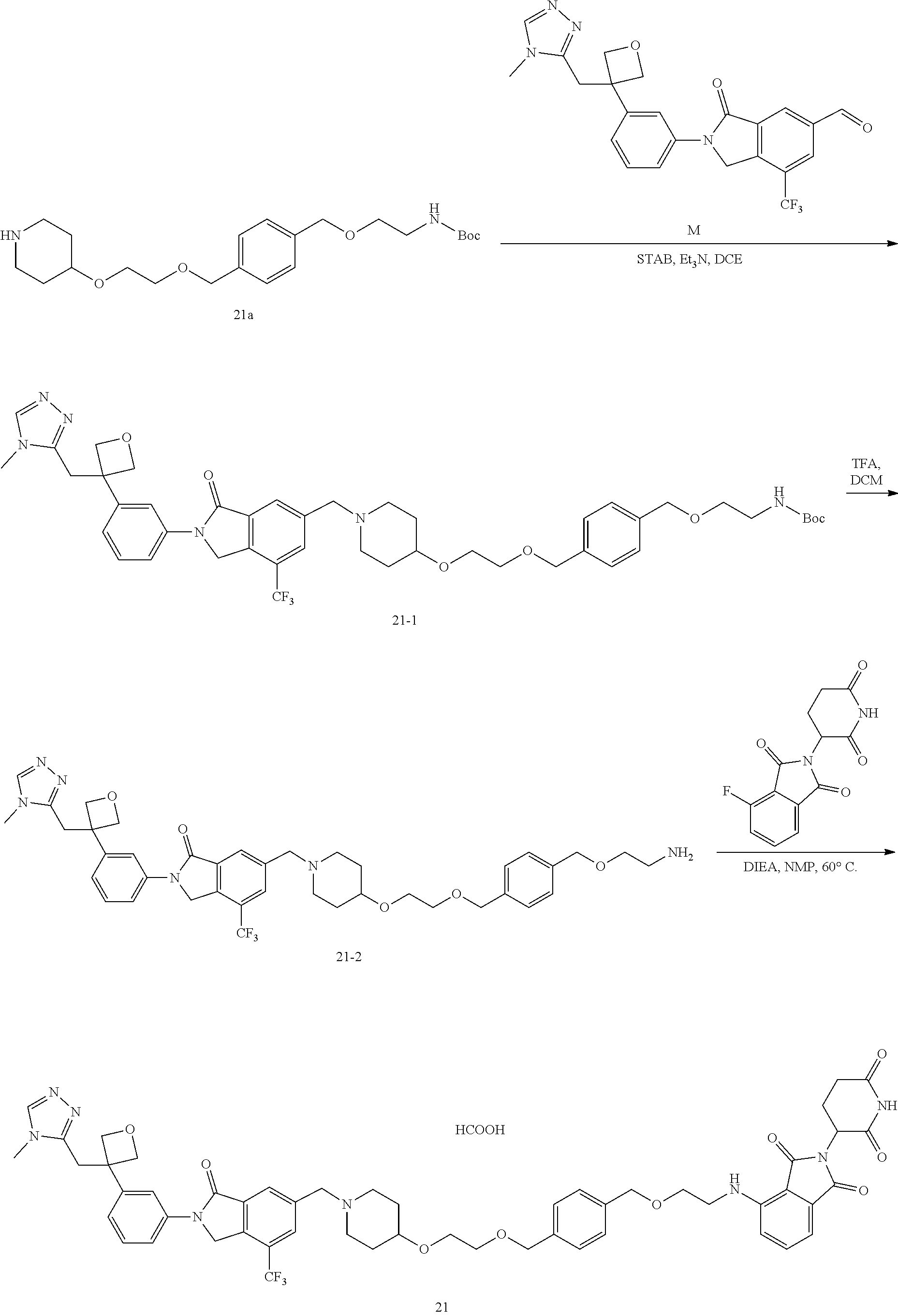

### Synthesis of Compound 21-1.

To a stirred solution of Intermediate M (700 mg, 1.534 mmol, 1 equiv) and Compound 21a (751.87 mg, 1.841 mmol, 1.2 equiv) in DCE (4 mL) were added Et₃N (155.20 mg, 1.534 mmol, 1 equiv) and STAB (650.09 mg, 3.068 mmol, 2 equiv). The resulting mixture was stirred for overnight at room temperature. The reaction was quenched by the addition of sat. NaHCO₃ (aq.) (15 mL). The resulting mixture was extracted with CH₂Cl₂/MeOH=10/1 (3×10 mL). The combined organic layers were concentrated under reduced pressure. The residue was purified by reverse flash chromatography with the following conditions: column, C18 silica gel; mobile phase, MeCN in water (10 mmol/L NH₄HCO₃), 15% to 75% gradient in 30 min; detector, UV 254 nm. This resulted in Compound 21-1 (420 mg, 32.26%) as a yellow solid.

### Synthesis of Compound 21-2.

To a stirred solution of Compound 21-1 (400 mg, 0.471 mmol, 1 equiv) in DCM (4 mL) was added TFA (1 mL) dropwise/in portions at room temperature. The resulting mixture was stirred for 3 h at room temperature. The resulting mixture was concentrated under reduced pressure. The residue was purified by Prep-TLC (CH2Cl2/MeOH 20:1) to afford Compound 21-2 (270 mg, 76.52%) as a yellow solid.

### Synthesis of Compound 21.

To a stirred solution of Compound 21-2 (260 mg, 0.347 mmol, 1 equiv) and 2-(2,6-dioxopiperidin-3-yl)-4-fluoroisoindole-1,3-dione (287.71 mg, 1.041 mmol, 3 equiv) in NMP (2 mL) was added DIEA (44.87 mg, 0.347 mmol, 1 equiv). The resulting mixture was stirred for overnight at 60° C. The reaction solution was purified by Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 25% B to 35% B in 7 min, 35% B; Wave Length: 254; 220 nm; RT1 (min): 6.38) to afford Compound 21 (66.0 mg, 18.09%) as a yellow solid.

LCMS-Compound 21: (ES, m/z): [M−HCOOH+H]⁺ 1005

NMR-Compound 21: (400 MHz, CD₃OD, δ ppm): δ1.70-1.81 (m, 2H), 1.88-1.94 (m, 5H), 2.08-2.14 (m, 1H), 2.50-2.52 (m, 2H), 2.74-2.78 (m, 1H), 2.80-2.93 (m, 6H), 3.49-3.52 (m, 3H), 3.63-3.73 (m, 8H), 3.92 (s, 21H), 4.54-4.58 (d, 4H), 5.06-5.11 (m, 7H), 6.77-6.79 (d, 1H), 7.00-7.03 (d, 1H), 7.32-7.37 (m, 4H), 7.40-7.42 (m, 2H), 7.44-7.51 (m, 1H), 7.75-7.77 (d, 1H), 7.99 (s, 1H), 8.07 (s, 1H), 8.20 (s, 1H), 8.35 (s, 1H).

### Example 20. Compound 22

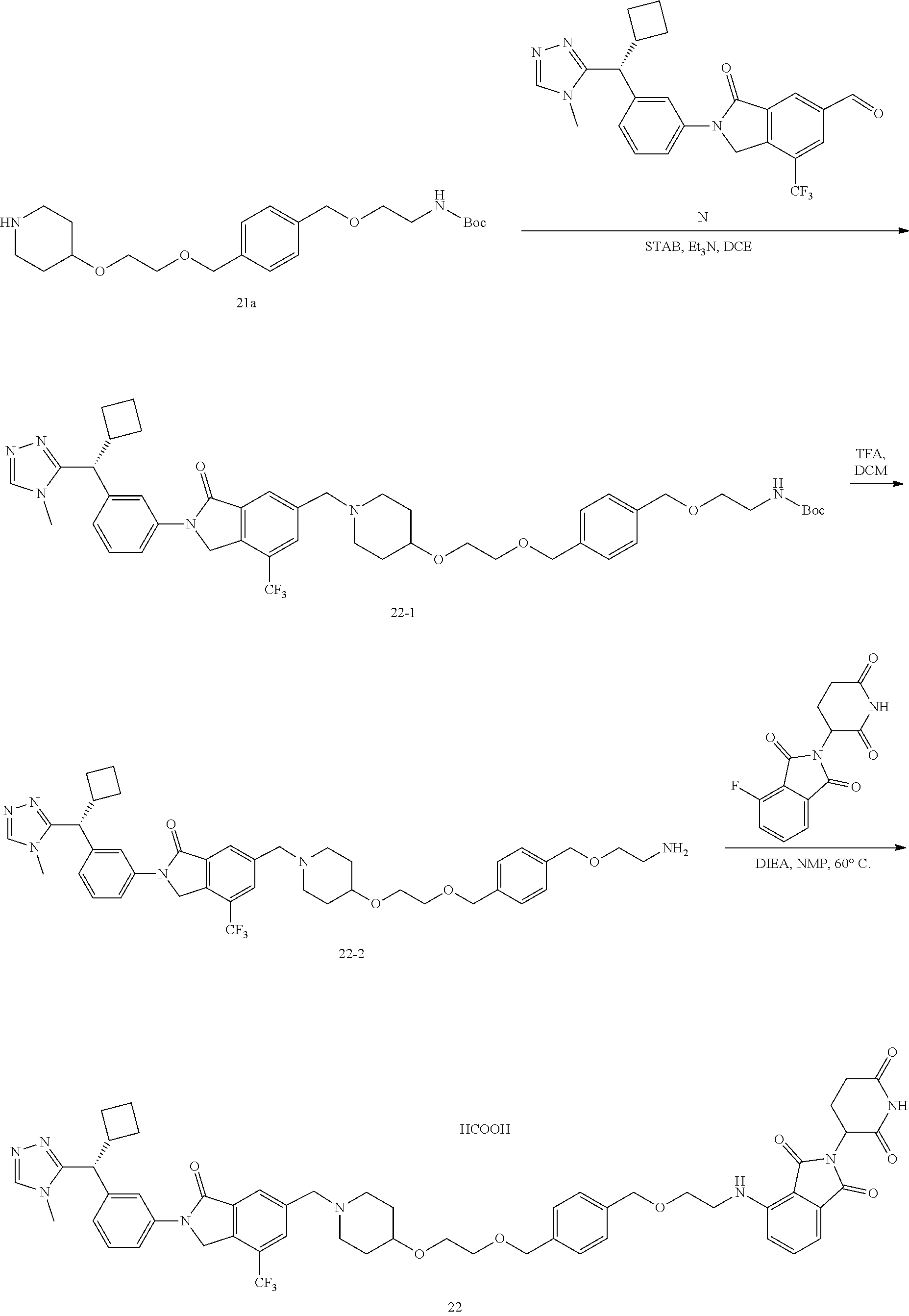

### Synthesis of Compound 22-1.

To a stirred mixture of Intermediate N (943.92 mg, 2.310 mmol, 1.5 equiv) and Compound 21a (700 mg, 1.540 mmol, 1.00 equiv) in DCE (15 mL) was added Et₃N (155.87 mg, 1.540 mmol, 1 equiv) and STAB (652.91 mg, 3.080 mmol, 2 equiv). The resulting mixture was stirred for overnight at room temperature. The reaction was quenched by the addition of sat. NaHCO₃ (aq.) (15 mL). The resulting mixture was extracted with CH₂Cl₂/MeOH=10/1 (2×20 mL). The combined organic layers were cowancentrated under reduced pressure. The residue was purified by reverse flash chromatography with the following conditions: column, C18 silica gel; mobile phase, MeCN in water (10 mmol/L NH₄HCO₃), 15% to 70% gradient in 30 min; detector, UV 254 nm. This resulted in Compound 22-1 (400 mg, 30.66%) as a yellow solid.

### Synthesis of Compound 22-2.

To a stirred solution of Compound 22-1 (400 mg, 0.472 mmol, 1 equiv) in DCM (4 mL) was added TFA (1 mL). The resulting mixture was stirred for 2 h at room temperature. The resulting mixture was concentrated under reduced pressure. The residue was purified by Prep-TLC (CH₂Cl₂/MeOH 12:1) to afford Compound 22-2 (240 mg, 68.04%) as a yellow solid.

### Synthesis of Compound 22.

To a stirred solution of Compound 22-2 (260 mg, 0.348 mmol, 1 equiv) and 2-(2,6-dioxopiperidin-3-yl)-4-fluoroisoindole-1,3-dione (288.47 mg, 1.044 mmol, 3 equiv) in NMP (2 mL) was added DIEA (44.99 mg, 0.348 mmol, 1 equiv). The resulting mixture was stirred for overnight at 60° C. The reaction solution was purified by Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 30% B to 40% B in 7 min, 40% B; Wave Length: 254; 220 nm; RT1 (min): 5.92) to afford Compound 22 (76.8 mg, 21.03%) as a yellow solid.

LCMS-Compound 22: (ES, m/z): [M−HCOOH+H]⁺ 1003

NMR-Compound 22: (400 MHz, CD₃OD, δ ppm): δ1.61-1.73 (m, 3H), 1.75-1.91 (m, 6H), 2.10-2.31 (m, 2H), 2.52-2.63 (m, 2H), 2.74-2.92 (m, 5H), 3.32-3.34 (m, 1H), 3.47-3.55 (m, 6H), 3.63-3.72 (m, 6H), 3.90 (s, 2H), 4.54-4.56 (d, 4H), 5.05-5.11 (m, 3H), 6.99-7.01 (d, 1H), 7.13-7.15 (d, 1H), 7.31-7.37 (m, 4H), 7.42-7.49 (m, 2H), 7.76-7.77 (m, 1H), 7.85 (s, 1H), 7.98 (s, 1H), 8.07 (s, 1H), 8.32 (s, 1H).

### Example 21. Compound 23

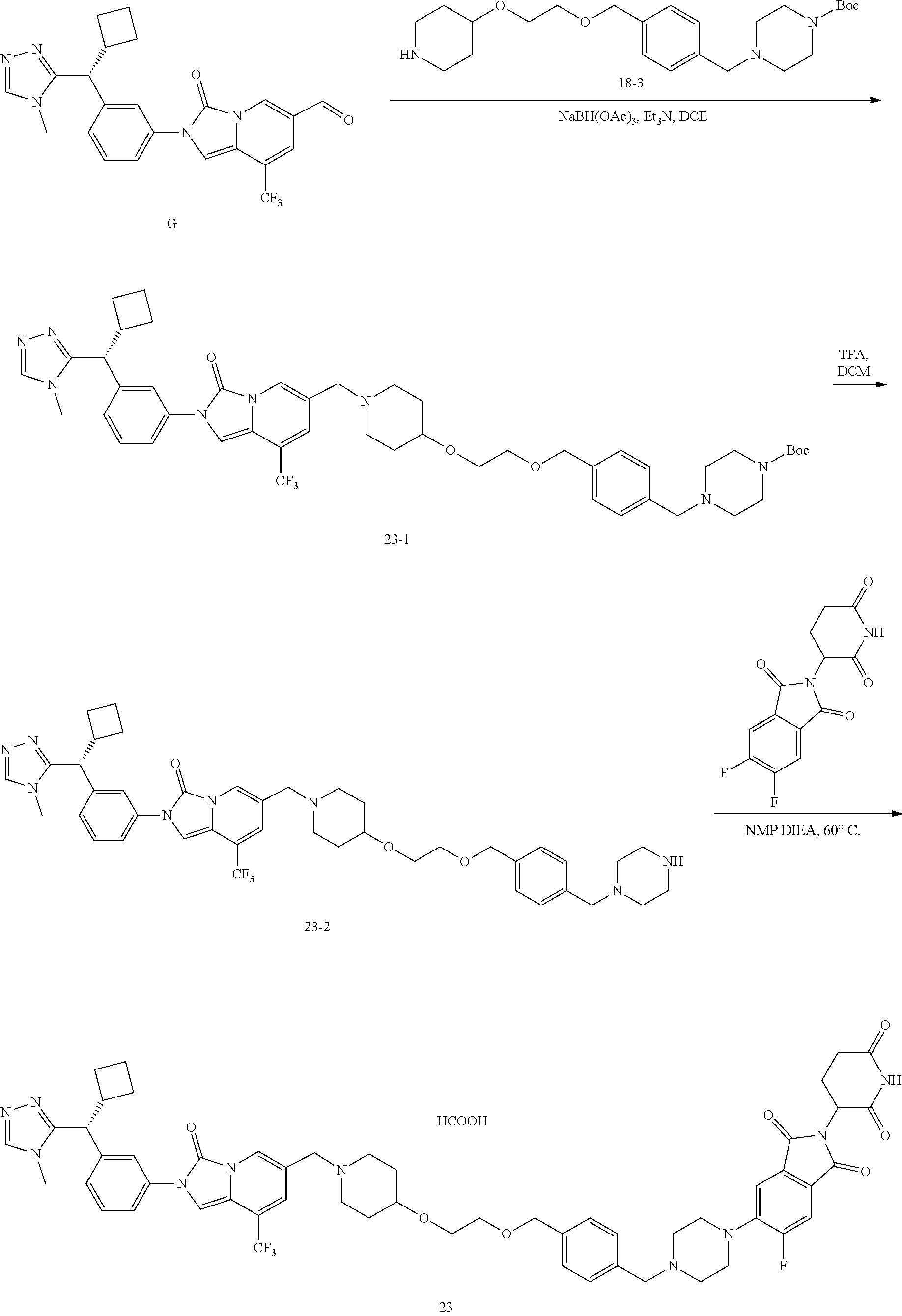

### Synthesis of Compound 23-1.

To a stirred solution of Intermediate G (1 g, 2.19 mmol, 1.0 equiv) and Compound 18-3 (0.95 g, 2.19 mmol, 1.0 equiv) in DCE (10 mL) was added Et₃N (0.22 g, 2.19 mmol, 1.0 equiv) at room temperature. The resulting mixture was stirred for 1 h at room temperature. To the above mixture was added NaBH(OAc)₃ (0.9 g, 4.39 mmol, 2.0 equiv) at room temperature. The resulting mixture was stirred for additional overnight at room temperature. The reaction was quenched by the addition of water (30 mL) at room temperature. The aqueous layer was extracted with CH₂Cl₂ (3×20 mL). The residue was purified by Prep-TLC (CH₂Cl₂/MeOH=10:1) to afford Compound 23-1 (850 mg, 44%) as a yellow solid.

### Synthesis of Compound 23-2.

A solution of Compound 23-1 (850 mg, 0.97 mmol, 1.0 equiv) and TFA (2 mL) in DCM (6 mL) was stirred for overnight at room temperature. The mixture was neutralized to pH 7 with NH₃ in MeOH. The resulting mixture was concentrated under reduced pressure. The residue was purified by Prep-TLC (CH₂Cl₂/MeOH=10:1) to afford Compound 23-2 (590 mg, 74%) as a yellow solid.

### Synthesis of Compound 23.

To a stirred solution of Compound 23-2 (200 mg, 0.25 mmol, 1.0 equiv) and 2-(2,6-dioxopiperidin-3-yl)-5,6-difluoroisoindole-1,3-dione (228 mg, 0.77 mmol, 3.0 equiv) in NMP (2 mL) was added DIEA (100 mg, 0.77 mmol, 3.0 equiv) at room temperature. The resulting mixture was stirred for overnight at 60° C. The reaction solution was purified by Prep-HPLC with the following conditions (Column: Xselec CSH C18 OBD Column 30*150 mm 5 μm, n; Mobile Phase A: water (0.1% FA), Mobile Phase B: ACN; Flow rate: 60 mL/min; Gradient: 14% B to 25% B in 10 min, 25% B; Wave Length: 254; 220 nm; RT1 (min): 9.77) to afford Compound 23 (74 mg, 26%) as a yellow solid.

LC-MS-Compound 23: (ES, m/z): [M-COOH]⁺ 1047

H-NMR-Compound 23: (400 MHz, CD₃OD, δ ppm): 1.74-1.96 (m, 1OH), 2.10-2.25 (m, 3H), 2.57-2.91 (m, 10H), 2.99-3.15 (m, 3H), 3.47-3.68 (m, 6H), 3.72 (s, 3H), 3.86 (s, 3H), 4.28-4.30 (d, 1H), 4.59 (s, 2H), 5.06-5.11 (m, 2H), 7.11-7.17 (m, 2H), 7.28-7.30 (m, 1H), 7.39 (s, 4H), 7.45-7.54 (m, 3H), 7.62-7.65 (m, 2H), 7.81 (s, 1H), 8.22-8.32 (m, 2H).

### Example 22. Compound 24

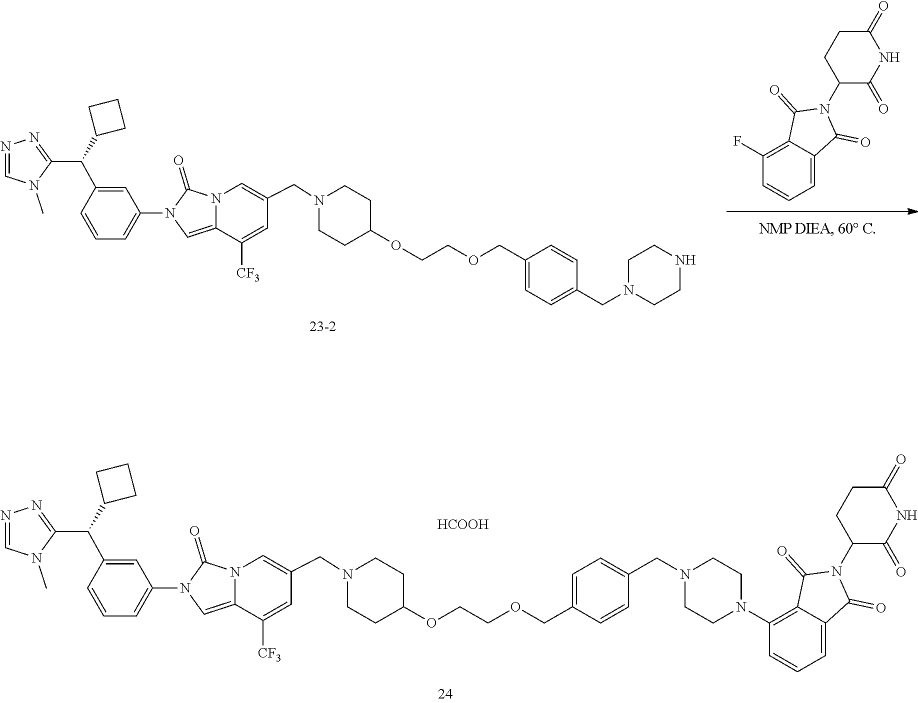 [SMILES: Cn1cnnc1[C@@H](c1cccc(-n2cc3c(C(F)(F)F)cc(CN4CCC(OCCOCc5ccc(CN6CCNCC6)cc5)CC4)cn3c2=O)c1)C1CCC1 ; O=C1CCC(N2C(=O)c3cccc(F)c3C2=O)C(=O)N1 ; Cn1cnnc1[C@@H](c1cccc(-n2cc3c(C(F)(F)F)cc(CN4CCC(OCCOCc5ccc(CN6CCN(c7cccc8c7C(=O)N(C7CCC(=O)NC7=O)C8=O)CC6)cc5)CC4)cn3c2=O)c1)C1CCC1 ; "HCOOH"]

### Synthesis of Compound 24.

To a stirred solution of Compound 23-2 (300 mg, 0.38 mmol, 1.0 equiv) and 2-(2,6-dioxopiperidin-3-yl)-4-fluoroisoindole-1,3-dione (321 mg, 1.16 mmol, 3.0 equiv) in NMP (3 mL) was added DIEA (150 mg, 1.16 mmol, 3.0 equiv) at room temperature. The resulting mixture was stirred for overnight at 60° C. The reaction solution was purified by Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 14% B to 25% B in 10 min, 25% B; Wave Length: 254; 220 min; RT1 (min): 9.27) to afford Compound 24 (56.9 mg, 13.19%) as a yellow solid.

LC-MS-Compound 24: (ES, m/z): [M-COOH]⁺ 1029

H-NMR-Compound 24: (400 MHz, CD₃OD, δ ppm): 1.77-1.97 (m, 1OH), 2.05-2.11 (m, 1H), 2.18-2.24 (m, 1H), 2.57-2.62 (m, 2H), 2.70-2.76 (m, 2H), 2.82-2.88 (m, 5H), 2.97-2.99 (m, 2H), 3.42 (s, 4H), 3.55 (s, 4H), 3.61 (s, 2H), 3.69 (s, 4H), 3.80 (s, 2H), 4.27-4.30 (d, 1H), 4.59 (s, 2H), 5.07-5.12 (m, 1H), 7.10-7.15 (d, 2H), 7.28-7.32 (t, 2H), 7.38-7.43 (m, 5H), 7.47-7.49 (t, 1H), 7.63-7.68 (m, 3H), 7.76 (s, 1H), 8.37 (s, 2H).

### Example 23. Compound 25

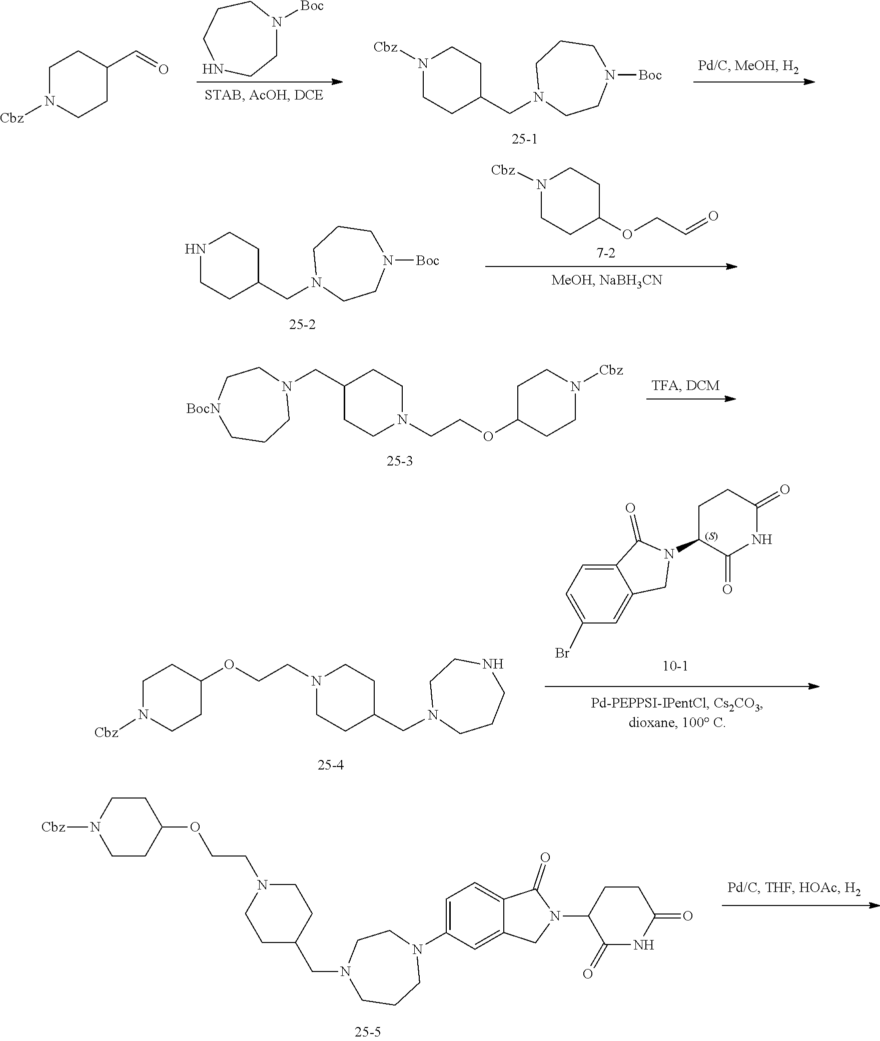

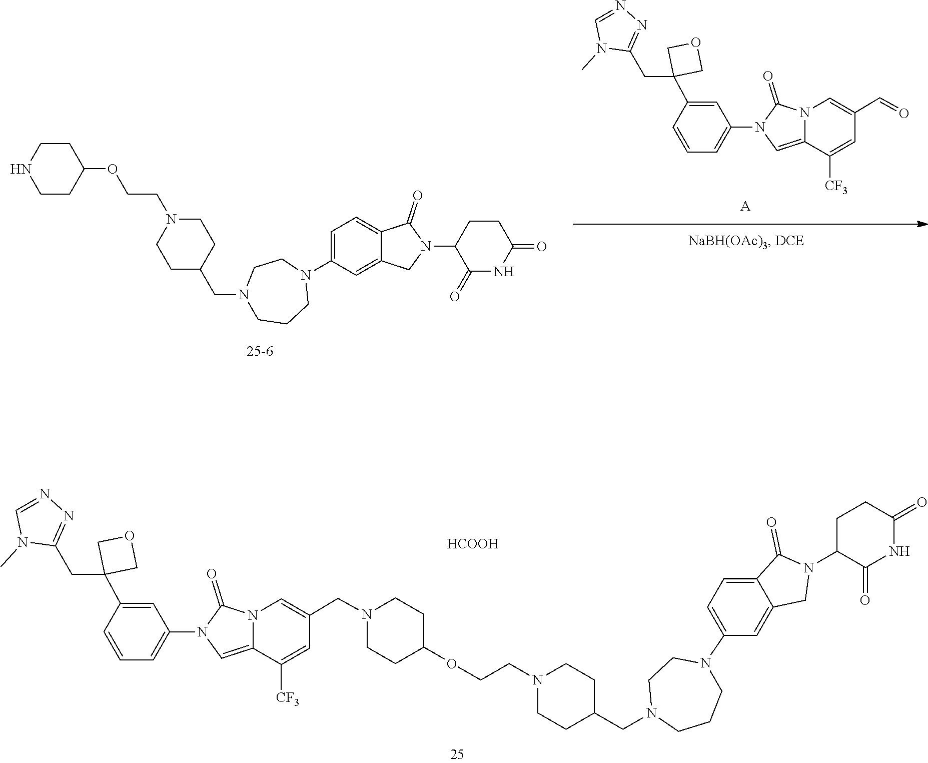

### Synthesis of Compound 25-1.

To a stirred solution of benzyl 4-formylpiperidine-1-carboxylate (10 g, 40.438 mmol, 1 equiv) and tert-butyl 1,4-diazepane-1-carboxylate (16.20 g, 80.876 mmol, 2 equiv) in DCM (100 mL) were added AcOH (2.43 g, 40.438 mmol, 1 equiv) and STAB (25.71 g, 121.314 mmol, 3 equiv) at room temperature. The resulting mixture was stirred for overnight at room temperature. The reaction was quenched by the addition of Water (200 mL) at room temperature. The resulting mixture was extracted with CH₂Cl₂ (3×200 mL). The combined organic layers were washed with water (3×200 mL), dried over anhydrous Na₂SO₄. After filtration, the filtrate was concentrated under reduced pressure. The residue was purified by silica gel column chromatography, eluted with PE/EA (5:1) to afford Compound 25-1 (17.5 g, 98.27%) as a white solid.

### Synthesis of Compound 25-2.

To a solution of Compound 25-1 (17.5 g, 40.549 mmol, 1 equiv) in 500 mL MeOH was added Pd/C (10%, 3.5 g) under nitrogen atmosphere in a 1 L round-bottom flask. The mixture was hydrogenated at room temperature for overnight under hydrogen atmosphere using a hydrogen balloon, filtered through a Celite pad and concentrated under reduced pressure. This resulted in Compound 25-2 (12 g, 89.54%) as a colorless oil.

### Synthesis of Compound 25-3.

To a stirred solution of Compound 7-2 (5 g, 18.030 mmol, 1 equiv) and Compound 25-2 (5.36 g, 18.030 mmol, 1 equiv) in MeOH (100 mL) was added NaBH₃CN (2.27 g, 36.060 mmol, 2 equiv). The resulting mixture was stirred for overnight at room temperature. The reaction was quenched by the addition of sat. NH₄Cl (aq.) (300 mL). The resulting mixture was extracted with EtOAc (3×200 mL). The combined organic layers were concentrated under reduced pressure. The residue was purified by reverse flash chromatography with the following conditions: column, C18 silica gel; mobile phase, MeCN in Water (10 mmol/L NH₄HFO₃), 30% to 100% gradient in 30 min; detector, UV 220 nm. This resulted in Compound 25-3 (4 g, 39.70%) as a white solid.

### Synthesis of Compound 25-4.

To a stirred solution of Compound 25-3 (4 g, 7.159 mmol, 1 equiv) in DCM (40 mL) was added TFA (5 mL). The resulting mixture was stirred for 4 h at room temperature. The resulting mixture was concentrated under reduced pressure. The residue was purified by reverse flash chromatography with the following conditions: column, C18 silica gel; mobile phase, MeCN in Water (10 mmol/L NH₄HCO₃), 5% to 60% gradient in 30 min; detector, UV 220 nm. This resulted in Compound 25-4 (2 g, 60.91%) as a white solid.

### Synthesis of Compound 25-5.

To a stirred mixture of benzyl Compound 25-4 (1 g, 2.180 mmol, 1 equiv) and Compound 10-1 (1.06 g, 3.270 mmol, 1.5 equiv) in dioxane (30 mL) were added Cs₂CO₃ (2.13 g, 6.540 mmol, 3 equiv) and Pd PEPPSI IPentCl (0.19 g, 0.218 mmol, 0.1 equiv) under argon atmosphere. The resulting mixture was stirred for overnight at 100° C. under argon atmosphere. The reaction was quenched by the addition of Water/HOAc=10/1 (100 mL). The resulting mixture was extracted with CH₂Cl₂/MeOH=8/1 (4×100 mL). The combined organic layers were concentrated under reduced pressure. The residue was purified by silica gel column chromatography, eluted with CH₂Cl₂/MeOH (8:1) to afford Compound 25-5 (1.08 g, 70.67%) as a light yellow solid.

### Synthesis of Compound 25-6.

To a solution of Compound 25-5 (1.08 g, 1.541 mmol, 1 equiv) in THF (20 mL) and HOAc (2 mL) was added Pd/C (150 mg, 20%) under nitrogen atmosphere in a 100 mL round-bottom flask. The mixture was hydrogenated at room temperature for overnight under hydrogen atmosphere using a hydrogen balloon, filtered through a Celite pad and concentrated under reduced pressure. This resulted in Compound 25-6 (420 mg, 48.09%) as a light yellow solid.

### Synthesis of Compound 25.

To a stirred solution of Compound 25-6 (400 mg, 0.706 mmol, 1 equiv) and Intermediate A (322.83 mg, 0.706 mmol, 1 equiv) in DCE (10 mL) were added ET₃N (71.42 mg, 0.706 mmol, 1 equiv) and NaBH(OAc)₃ (224.38 mg, 1.059 mmol, 1.5 equiv) at room temperature under nitrogen atmosphere. The resulting mixture was stirred for overnight at room temperature under nitrogen atmosphere. The reaction was quenched by the addition of Water/HOAc=10/1 (20 mL) at room temperature. The resulting mixture was extracted with CH₂Cl₂/MeOH=10/1 (3×mL). The aqueous layer was concentrated under reduced pressure. The residue was purified by Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 9% B to 22% B in 7 min, 22% B; Wave Length: 254; 220 nm; RT1 (min): 6.25) to afford Compound 25 (37.1 mg, 5.21%) as a yellow solid.

LCMS-Compound 25: (ES, m/z): [M−HCOOH+H]⁺ 1008

NMR-Compound 25: (400 MHz, CD₃OD, δ ppm): δ1.25-1.42 (m, 2H), 1.62-1.76 (m, 5H), 1.83-2.02 (m, 4H), 2.10-2.21 (m, 1H), 2.46-2.53 (m, 5H), 2.80-2.97 (m, 13H), 3.29-3.32 (m, 2H), 3.49-3.58 (m, 5H), 3.64-3.69 (m, 4H), 3.75-3.80 (m, 4H), 4.37-4.40 (m, 2H), 5.07-5.13 (m, 5H), 6.89-6.91 (m, 2H), 6.94-6.96 (d, 1H), 7.12-7.15 (m, 2H), 7.29 (s, 1H), 7.47-7.50 (t, 1H), 7.59-7.64 (m, 2H), 7.76 (s, 1H), 8.20 (s, 1H), 8.35 (s, 2H).

### Example 24. Compound 26

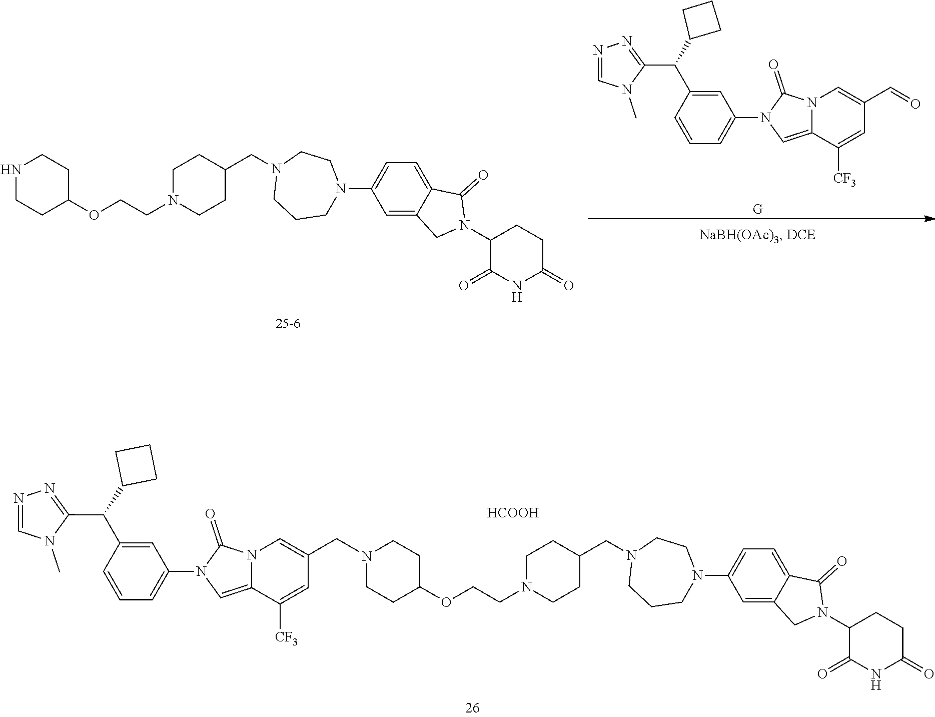

### Synthesis of Compound 26.

To a stirred solution of Compound 25-6 (300 mg, 0.529 mmol, 1 equiv) and Intermediate G (241.08 mg, 0.529 mmol, 1 equiv) in DCE (9 mL) were added Et₃N (53.57 mg, 0.529 mmol, 1 equiv) and NaBH(OAc)₃ (168.28 mg, 0.794 mmol, 1.5 equiv). The resulting mixture was stirred for overnight at room temperature under nitrogen atmosphere. The reaction was quenched by the addition of Water/HOAc=10/1 (30 mL). The resulting mixture was extracted with CH₂Cl₂/MeOH=10/1 (3/10×mL). The aqueous layer was concentrated under reduced pressure. The residue was purified by Prep-HPLC with the following conditions (Column: XSelect CSH Prep C18 OBD Column, 19*250 mm, 5 μm; Mobile Phase A: Water (0.1% FA), Mobile Phase B: ACN; Flow rate: 30 mL/min; Gradient: 4% B to 15% B in 8 min, 15% B; Wave Length: 254; 220 nm; RT1 (min): 7.40) to afford Compound 26 (69.1 mg, 12.41%) as a yellow solid.

LCMS-Compound 26: (ES, m/z): [M−HCOOH+H]⁺ 1007

NMR-Compound 26: (400 MHz, CD₃OD, δ ppm): δ1.25-1.42 (m, 2H), 1.71-1.98 (m, 14H), 2.11-2.20 (m, 1H), 2.22-2.29 (m, 1H), 2.43-2.52 (m, 5H), 2.75-2.81 (m, 3H), 2.83-2.92 (m, 7H), 3.29-3.32 (m, 4H), 3.49-3.55 (m, 5H), 3.56 (s, 3H), 3.64-3.66 (m, 4H), 3.73-3.80 (m, 4H), 4.28-4.32 (m, 1H), 4.38-4.40 (m, 2H), 5.07-5.13 (m, 1H), 6.89-6.90 (m, 2H), 7.11 (s, 1H), 7.15 (s, 1H), 7.29-7.31 (m, 1H), 7.51-7.53 (t, 1H), 7.59-7.61 (m, 1H), 7.64-7.66 (m, 2H), 7.73 (s, 1H), 8.30 (s, 2H), 8.32 (s, 1H).

### Example 25. Compound 27

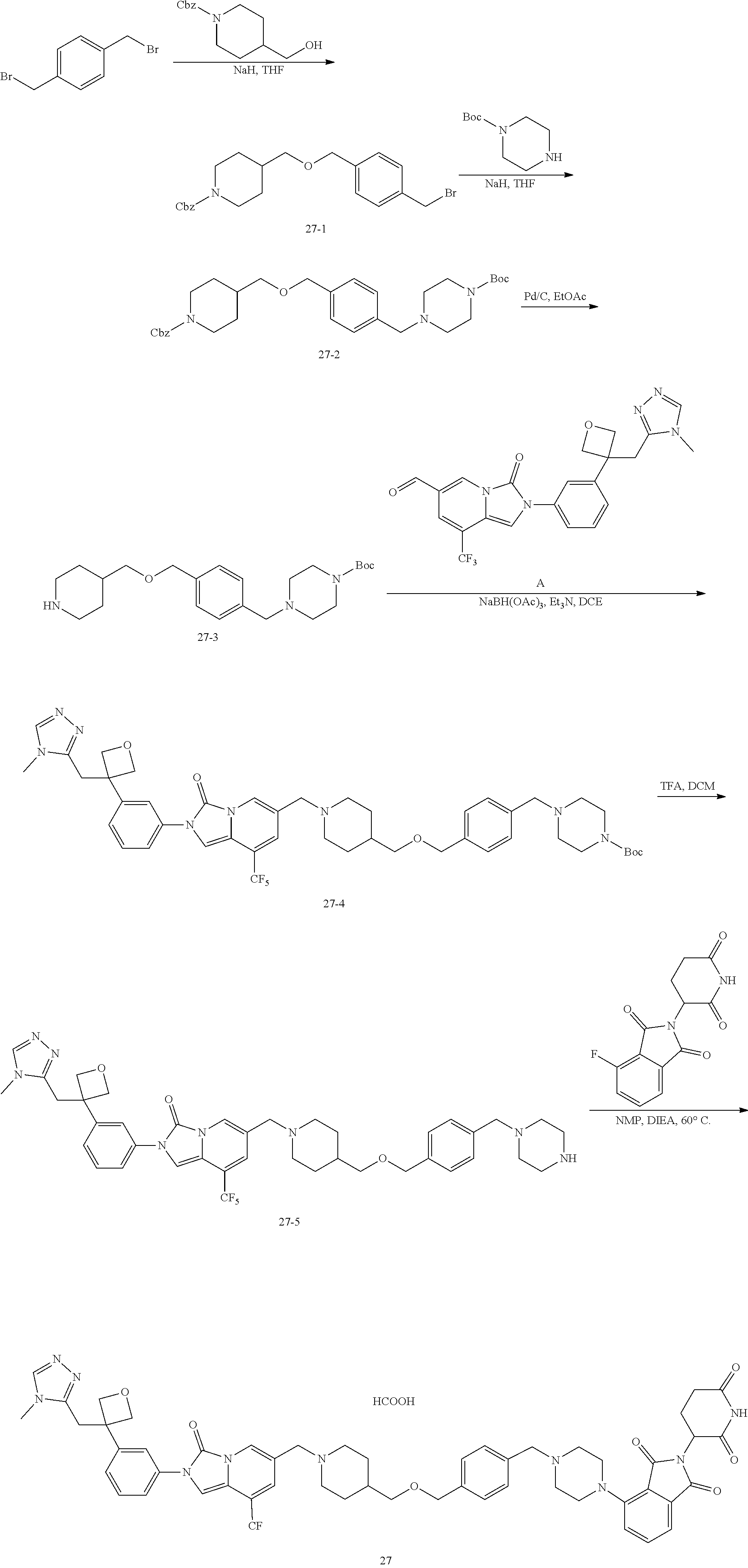

### Synthesis of Compound 27-1.

To a solution of benzyl 4-(hydroxymethyl)piperidine-1-carboxylate (10 g, 40.111 mmol, 1 equiv) in THF (100 mL) was added sodium hydride (60% in oil, 1.93 g) at 0 degrees C. The mixture was stirred for 30 min. To the above mixture 1,4-bis(bromomethyl)-benzene (15.88 g, 60.166 mmol, 1.5 equiv) was added. The resulting mixture was stirred for overnight at room temperature. The reaction mixture was quenched by water (300 mL) and extracted with DCM (3×250 mL). The resulting mixture was concentrated under reduced pressure. The residue was purified by silica gel column chromatography, eluted with PE/EA (0.5:1) to afford Compound 27-1 (9 g, 51.90%) as a colorless oil.

### Synthesis of Compound 27-2.

Into a 1000 mL 3-necked round-bottom flask were added tert-butyl piperazine-1-carboxylate (6.46 g, 34.694 mmol, 1.5 equiv), THF (150 mL) and NaH (0.67 g, 27.755 mmol, 1.2 equiv, 60% in oil) at 0° C. The resulting mixture was stirred for 1 h at 0° C. To the above mixture was added Compound 27-1 (10 g, 23.129 mmol, 1 equiv) at 0° C. The resulting mixture was stirred for additional 4 h at room temperature. The reaction was quenched with 300 mL NH₄Cl (a.q.) at room temperature. The resulting mixture was extracted with EtOAc (3×200 mL). The resulting mixture was concentrated under reduced pressure. The residue was purified by silica gel column chromatography, eluted with PE:EA (10:1) to afford Compound 27-2 (3.3 g, 26.53%) as a light yellow solid.

### Synthesis of Compound 27-3.

To a solution of Compound 27-2 (3 g, 5.579 mmol, 1 equiv) in 50 mL EtOAc was added Pd/C (10%, 0.6 g) under nitrogen atmosphere in a 250 mL round-bottom flask. The mixture was hydrogenated at room temperature for 20 min under hydrogen atmosphere using a hydrogen balloon, filtered through a Celite pad and concentrated under reduced pressure. This resulted in Compound 27-3 (1.8 g, 79.94%) as a light yellow solid.

### Synthesis of Compound 27-4.

To a stirred solution of Compound 27-3 (794.05 mg, 1.968 mmol, 1 equiv) and Intermediate A (900 mg, 1.968 mmol, 1.00 equiv) in DCE (20 mL) was added Et₃N (199.11 mg, 1.968 mmol, 1 equiv) and NaBH(OAc)3 (834.02 mg, 3.936 mmol, 2 equiv). The resulting mixture was stirred for overnight at room temperature. The reaction was quenched by the addition of sat. NaHCO₃ (aq.) (20 mL) at room temperature. The resulting mixture was extracted with CH₂Cl₂/MeOH=10/1 (2×30 mL). The combined organic layers were concentrated under reduced pressure. The residue was purified by reverse flash chromatography with the following conditions: column, C18 silica gel; mobile phase, MeCN in water (10 mmol/L NH₄HCO₃), 25% to 90% gradient in 30 min; detector, UV 254 nm. This resulted in Compound 27-4 (850 mg, 51.13%) as a yellow solid.

### Synthesis of Compound 27-5.

To a stirred solution of Compound 27-4 (850 mg, 1.006 mmol, 1 equiv) in DCM (10 mL) was added TFA (2 mL). The resulting mixture was stirred for 4 h at room temperature. The resulting mixture was concentrated under reduced pressure. The residue was purified by Prep-TLC (CH₂Cl₂/MeOH 10:1) to afford Compound 27-5 (680 mg, 90.75%) as a yellow solid.

### Synthesis of Compound 27.

To a stirred solution of Compound 27-5 (300 mg, 0.403 mmol, 1 equiv) and 2-(2,6-dioxopiperidin-3-yl)-4-fluoroisoindole-1,3-dione (333.75 mg, 1.209 mmol, 3 equiv) in NMP (3 mL) was added DIEA (52.06 mg, 0.403 mmol, 1 equiv). The resulting mixture was stirred for overnight at 60° C. The reaction solution was purified by Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 10% B to 22% B in 9 min, 22% B; Wave Length: 220 nm; RT1 (min): 9.08) to afford Compound 27 (129 mg, 30.59%) as a yellow solid.

LCMS-Compound 27: (ES, m/z): [M−HCOOH+H]⁺ 1001

NMR-Compound 27: (400 MHz, CD₃OD, δ ppm): δ1.50-1.54 (m, 2H), 1.86-1.95 (m, 3H), 2.10-2.13 (m, 1H), 2.63-2.97 (m, 12H), 3.29-3.32 (m, 2H), 3.43-3.56 (m, 6H), 3.68 (s, 2H), 3.71-3.88 (m, 4H), 4.54 (s, 2H), 5.08-5.13 (m, 5H), 6.94-6.96 (d, 1H), 7.12-7.17 (m, 2H), 7.29-7.50 (m, 8H), 7.62-7.71 (m, 2H), 7.85 (s, 1H), 8.20 (s, 1H), 8.36 (s, 2H).

### Example 26. Compound 28

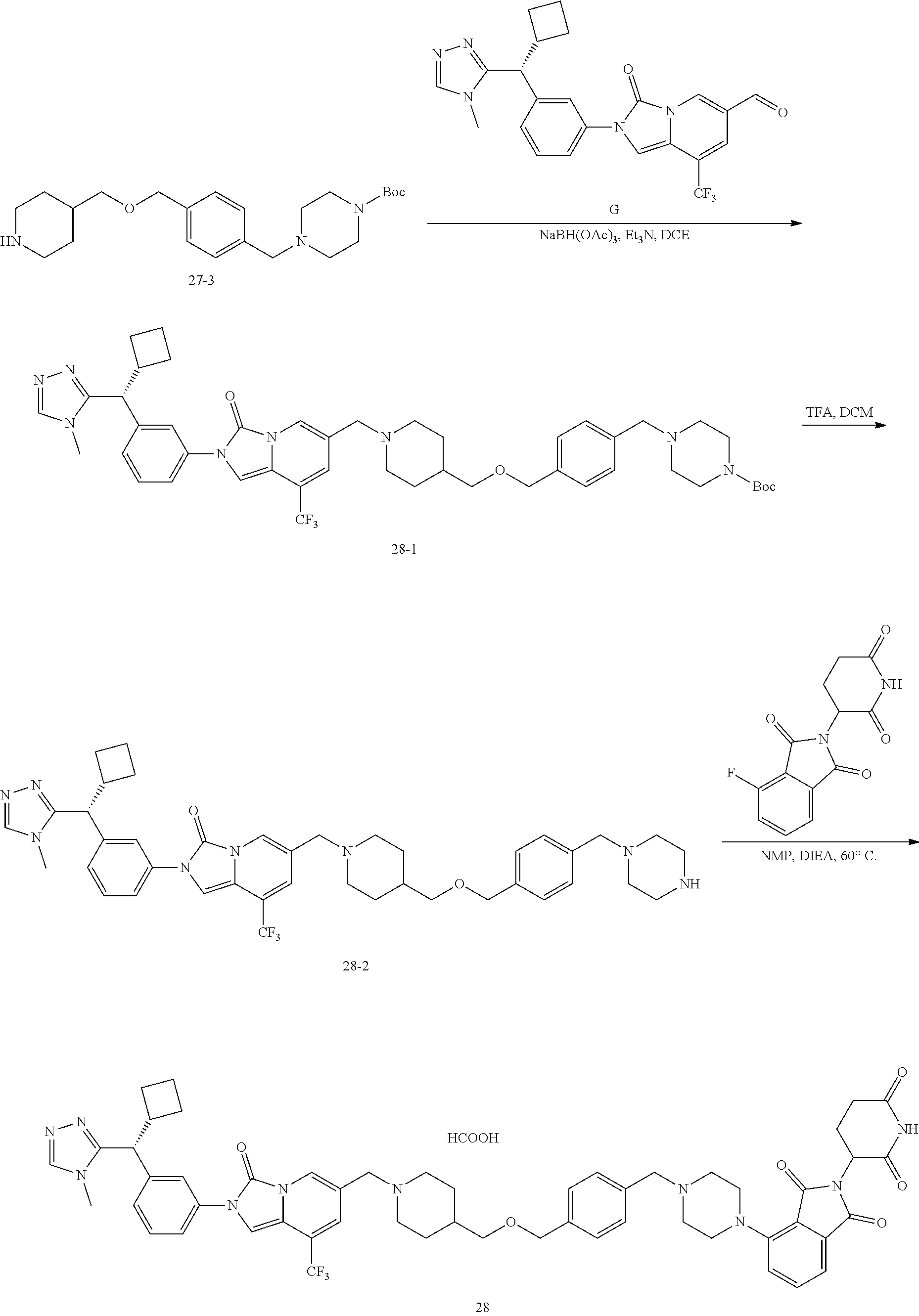

### Synthesis of Compound 28-1.

To a stirred solution of Compound 27-3 (708.88 mg, 1.757 mmol, 1 equiv) and Intermediate G (800 mg, 1.757 mmol, 1.00 equiv) in DCE (15 mL) was added Et₃N (177.75 mg, 1.757 mmol, 1 equiv) and NaBH(OAc)₃ (744.56 mg, 3.514 mmol, 2 equiv). The resulting mixture was stirred for overnight at room temperature. The reaction was quenched by the addition of sat. NaHCO₃ (aq.) (20 mL) at room temperature. The resulting mixture was extracted with CH₂Cl₂ (3×20 mL). The combined organic layers were concentrated under reduced pressure. The residue was purified by reverse flash chromatography with the following conditions: column, C18 silica gel; mobile phase, MeCN in water (10 mmol/L NH₄HCO₃), 25% to 90% gradient in 30 min; detector, UV 254 nm. This resulted in Compound 28-1 (800 mg, 54.03%) as a yellow solid.

### Synthesis of Compound 28-2.

To a stirred solution of Compound 28-1 (800 mg, 0.949 mmol, 1 equiv) in DCM (5 mL) was added TFA (1 mL). The resulting mixture was stirred for 3 h at room temperature. The resulting mixture was concentrated under reduced pressure. The residue was purified by Prep-TLC (CH₂Cl₂/MeOH 10:1) to afford Compound 28-2 (650 mg, 92.20%) as a yellow solid.

### Synthesis of Compound 28.

To a stirred solution of Compound 28-2 (300 mg, 0.404 mmol, 1 equiv) and 2-(2,6-dioxopiperidin-3-yl)-4-fluoroisoindole-1,3-dione (334.64 mg, 1.212 mmol, 3 equiv) in NMP (3 mL) was added DIEA (52.19 mg, 0.404 mmol, 1 equiv). The resulting mixture was stirred for overnight at 60° C. The reaction solution was purified by Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 20% B to 35% B in 7 min, 35% B; Wave Length: 220 nm; RT1 (min): 4.5) to afford Compound 28 (114 mg, 27.01%) as a yellow solid.

LCMS-Compound 28: (ES, m/z): [M−HCOOH+H]⁺ 999

NMR-Compound 28: (400 MHz, CD₃OD, δ ppm): δ1.38-1.47 (m, 2H), 1.76-1.91 (m, 8H), 2.10-2.13 (m, 1H), 2.26-2.36 (m, 3H), 2.70-2.87 (m, 6H), 3.10-3.10 (m, 2H), 3.29-3.32 (m, 1H), 3.39-3.41 (m, 6H), 3.51-3.59 (m, 5H), 3.72 (s, 2H), 4.28-4.30 (d, 1H), 4.53 (s, 2H), 5.08-5.13 (m, 1H), 7.11-7.14 (m, 2H), 7.27-7.40 (m, 7H), 7.48-7.52 (t, 1H), 7.63-7.69 (m, 3H), 7.75 (s, 1H), 8.35 (s, 1H), 8.44 (s, 1H).

### Example 27. Compound 29

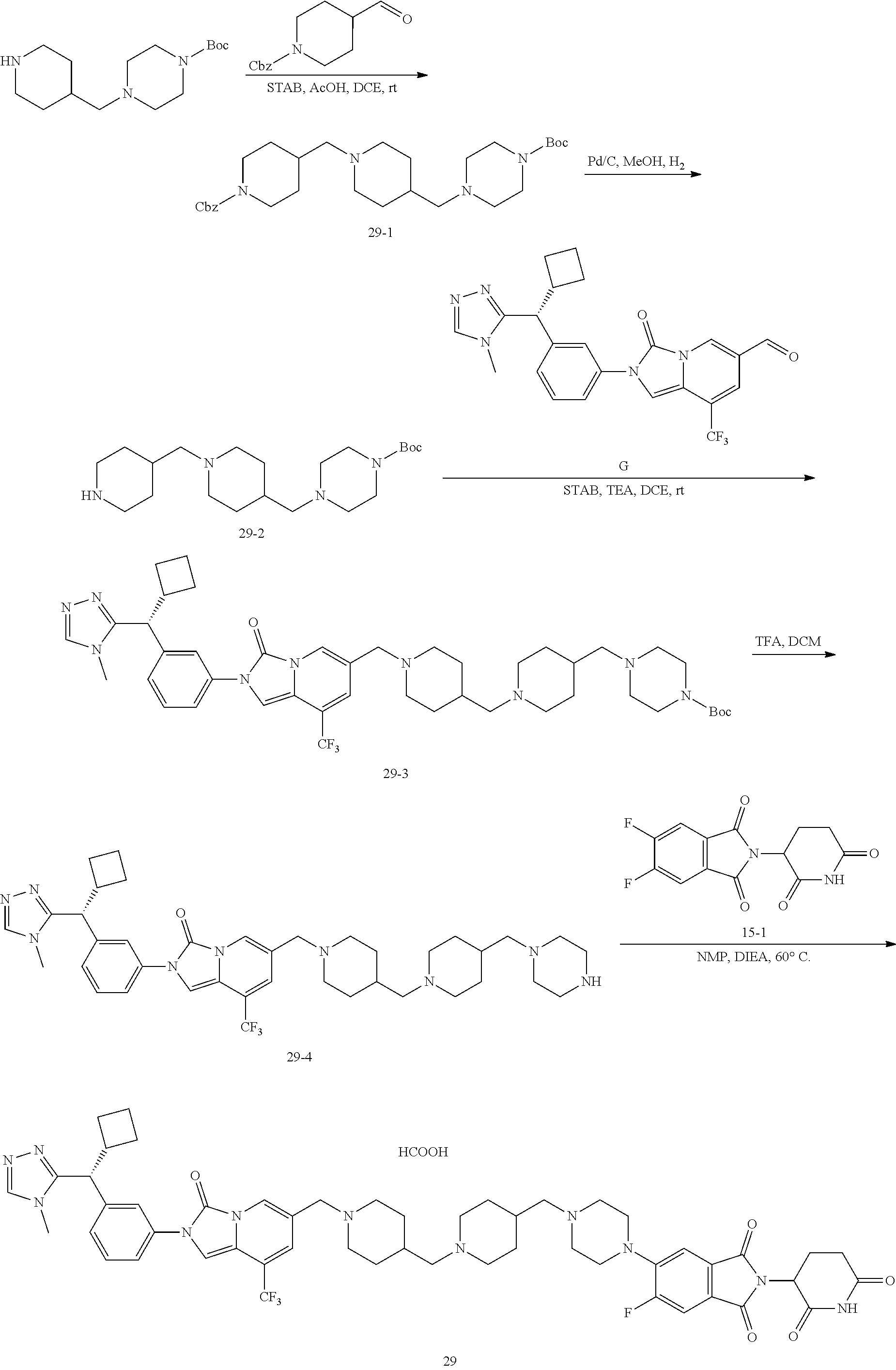

### Synthesis of Compound 29-1.

A solution of tert-butyl 4-(piperidin-4-ylmethyl)piperazine-1-carboxylate (3 g, 10.585 mmol, 1 equiv) and benzyl 4-formylpiperidine-1-carboxylate (2.62 g, 10.585 mmol, 1 equiv) in DCE (30 mL) was treated with AcOH (0.64 g, 10.585 mmol, 1 equiv) for 30 min at room temperature under nitrogen atmosphere followed by the addition of STAB (4.49 g, 21.170 mmol, 2 equiv) at room temperature. The resulting mixture was stirred for additional overnight at room temperature. The reaction was quenched with sat. NH₄Cl (aq.) (100 mL) at room temperature. The aqueous layer was extracted with CH₂Cl₂:MeOH (5:1) (3×30 mL). The resulting mixture was concentrated under reduced pressure. The residue was purified by silica gel column chromatography, eluted with CH₂Cl₂/MeOH (10:1) to afford Compound 29-1 (2.5 g, 41.76%) as a brown solid.

### Synthesis of Compound 29-2.

To a solution of Compound 29-1 (2.5 g, 4.857 mmol, 1 equiv) in 25 mL MeOH was added Pd/C (10%, 0.6 g) under nitrogen atmosphere in a 250 mL round-bottom flask. The mixture was hydrogenated at room temperature for 2 h under hydrogen atmosphere using a hydrogen balloon, filtered through a Celite pad and concentrated under reduced pressure. The resulting mixture was filtered, the filter cake was washed with CH₂Cl₂ (3×10 mL). The filtrate was concentrated under reduced pressure. This resulted in Compound 29-2 (1.6 g, 86.56%) as a off-white solid.

### Synthesis of Compound 29-3.

To a stirred solution of Compound 29-2 (600 mg, 1.577 mmol, 1 equiv) and Intermediate G (718.03 mg, 1.577 mmol, 1 equiv) in DCE (15 mL) was added TEA (159.54 mg, 1.577 mmol, 1 equiv). To the above mixture was added STAB (668.27 mg, 3.154 mmol, 2 equiv) at room temperature. The resulting mixture was stirred for overnight at room temperature. The reaction was quenched by the addition of sat. NaHCO₃ (aq.) (20 mL). The resulting mixture was extracted with CH₂Cl₂/MeOH=10/1 (3×20 mL). The combined organic layers were concentrated under reduced pressure. The residue was purified by reverse flash chromatography with the following conditions: column, C18 silica gel; mobile phase, MeCN in water (10 mmol/L NH₄HCO₃), 25% to 100% gradient in 30 min; detector, UV 254 nm. This resulted in Compound 29-3 (750 mg, 58.01%) as a yellow solid.

### Synthesis of Compound 29-4.

To a stirred solution of Compound 29-3 (740 mg, 0.902 mmol, 1 equiv) in DCM (5 mL) was added TFA (1 mL). The resulting mixture was stirred for 4 h at room temperature. The resulting mixture was concentrated under reduced pressure. The residue was purified by Prep-TLC (CH2Cl2/MeOH 10:1) to afford Compound 29-4 (550 mg, 84.66%) as a yellow solid.

### Synthesis of Compound 29.

To a stirred solution of Compound 29-4 (300 mg, 0.417 mmol, 1 equiv) and Compound 15-1 (367.82 mg, 1.251 mmol, 3 equiv) in NMP (3 mL) was added DIEA (53.86 mg, 0.417 mmol, 1 equiv). The resulting mixture was stirred for overnight at room temperature. The reaction solution was purified by Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 8% B to 20% B in 8 min, 20% B; Wave Length: 220 nm; RT1 (min): 7.12) to afford Compound 29 (194 mg, 44.76%) as a yellow solid.

LCMS-Compound 29: (ES, m/z): [M−HCOOH+H]⁺ 994

NMR-Compound 29: (400 MHz, CD₃OD, δ ppm): δ1.40-1.53 (m, 4H), 1.83-1.92 (m, 9H), 2.05-2.25 (m, 6H), 2.35-2.37 (m, 2H), 2.66-3.05 (m, 14H), 3.32-3.33 (m, 4H), 3.29-3.32 (m, 1H), 3.42 (s, 2H), 3.53-3.56 (m, 5H), 4.29-4.31 (d, 1H), 5.08-5.13 (m, 1H), 7.10-7.14 (d, 2H), 7.29-7.31 (m, 1H), 7.48-7.58 (m, 3H), 7.64-7.69 (m, 3H), 8.35 (s, 1H), 8.44 (s, 1H).

### Example 28. Compound 30

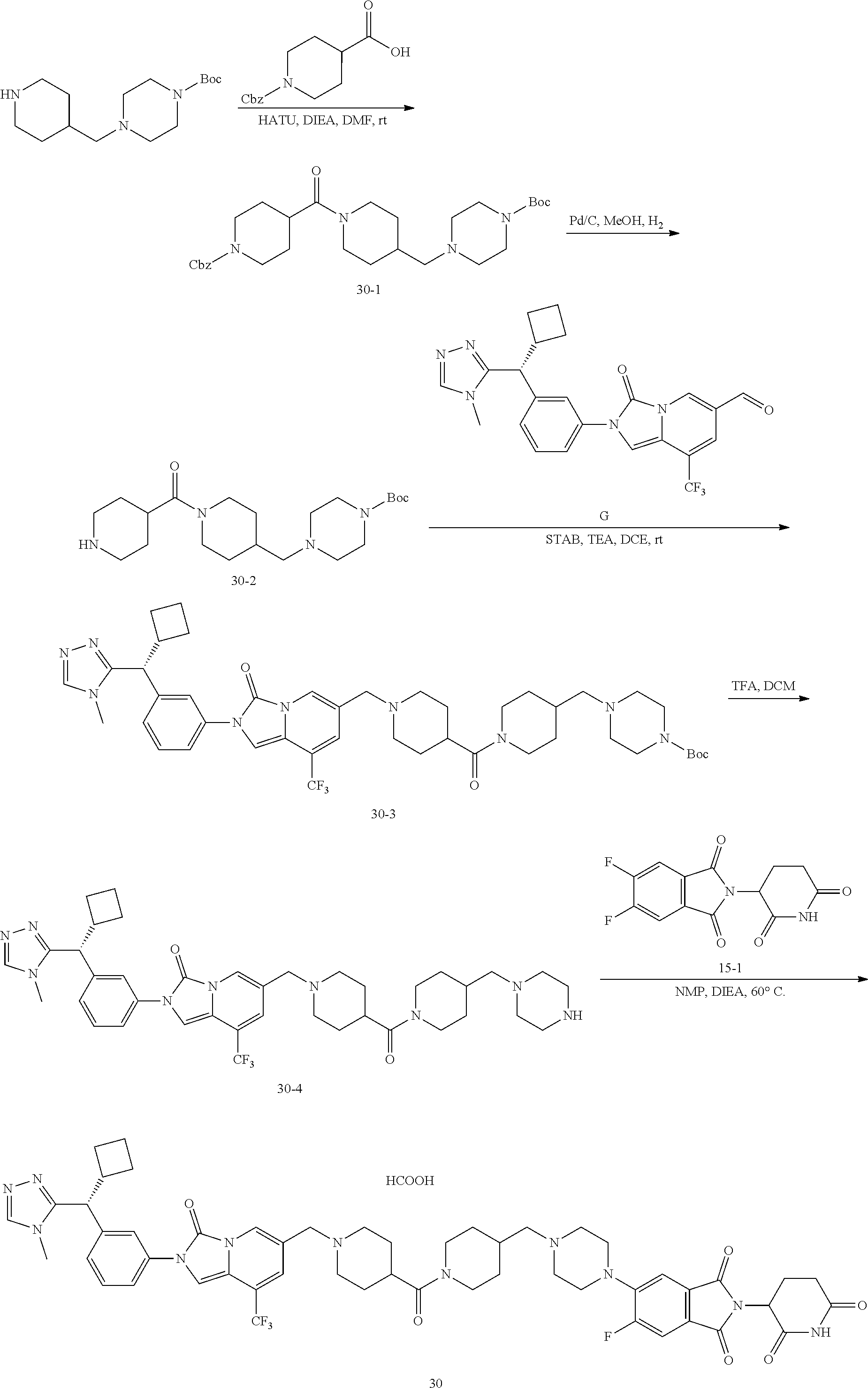

### Synthesis of Compound 30-1.

To a stirred solution of 1-[(benzyloxy)carbonyl]piperidine-4-carboxylic acid (3.68 g, 13.973 mmol, 1.2 equiv) and HATU (6.64 g, 17.466 mmol, 1.5 equiv) in DMF (35 mL) were added DIEA (3.01 g, 23.288 mmol, 2 equiv) and tert-butyl 4-(piperidin-4-ylmethyl)piperazine-1-carboxylate (3.3 g, 11.644 mmol, 1 equiv) at room temperature under nitrogen atmosphere. The resulting mixture was stirred for additional overnight at room temperature. The reaction was quenched with Water/Ice (100 mL) at room temperature. The resulting mixture was extracted with EtOAc (2×30 mL). The combined organic layers were washed with sat. NaCl (aq.) (2×30 mL). After filtration, the filtrate was concentrated under reduced pressure. The residue was purified by reverse flash chromatography with the following conditions: column, C18 silica gel; mobile phase, MeCN in Water (10 mmol/L NH4HCO₃), 10% to 85% gradient in 50 min; detector, UV 220 nm. This resulted in Compound 30-1 (2.7 g, 40.79%) as a brown solid.

### Synthesis of Compound 30-2.

To a solution of Compound 30-1 (2.7 g, 5.107 mmol, 1 equiv) in 100 mL MeOH was added Pd/C (10%, 0.6 g) under nitrogen atmosphere in a 250 mL round-bottom flask. The mixture was hydrogenated at room temperature for 2 h under hydrogen atmosphere using a hydrogen balloon, filtered through a Celite pad and concentrated under reduced pressure. The resulting mixture was filtered, the filter cake was washed with CH2Cl2 (3×30 mL). The filtrate was concentrated under reduced pressure. The crude product was used in the next step directly without further purification. This resulted in Compound 30-2 (1.56 g, 77.42%) as a grey solid.

### Synthesis of Compound 30-3.

To a stirred solution of Compound 30-2 (519.80 mg, 1.318 mmol, 1.2 equiv) and Intermediate G (500 mg, 1.098 mmol, 1.00 equiv) in DCE (10 mL) was added TEA (111.09 mg, 1.098 mmol, 1 equiv) and STAB (465.35 mg, 2.196 mmol, 2 equiv). The resulting mixture was stirred for overnight at room temperature. The reaction was quenched by the addition of sat. NaHCO₃ (aq.) (20 mL). The resulting mixture was extracted with CH₂Cl₂/MeOH=10/1 (2×20 mL). The combined organic layers were concentrated under reduced pressure. The residue was purified by reverse flash chromatography with the following conditions: column, C18 silica gel; mobile phase, MeCN in water (10 mmol/L NH₄HCO₃), 20% to 90% gradient in 30 min; detector, UV 254 nm. This resulted in Compound 30-3 (550 mg, 60.07%) as a yellow solid.

### Synthesis of Compound 30-4.

To a stirred solution of Compound 30-3 (550 mg, 0.659 mmol, 1 equiv) in DCM (5 mL) was added TFA (1 mL). The resulting mixture was stirred for 4 h at room temperature. The resulting mixture was concentrated under reduced pressure. The residue was purified by Prep-TLC (CH₂Cl₂/MeOH 10:1) to afford Compound 30-4 (450 mg, 92.98%) as a yellow solid.

### Synthesis of Compound 30.

To a stirred solution of Compound 30-4 (250 mg, 0.341 mmol, 1 equiv) and Compound 15-1 (300.67 mg, 1.023 mmol, 3 equiv) in NMP (3 mL) was added DIEA (44.03 mg, 0.341 mmol, 1 equiv). The resulting mixture was stirred for overnight at room temperature. The reaction solution was purified by Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 10% B to 27% B in 7 min, 27% B; Wave Length: 220 nm; RT1 (min): 6.53) to afford Compound 30 (183 mg, 50.96%) as a yellow solid.

LCMS-Compound 30: (ES, m/z): [M−HCOOH+H]⁺ 1008

NMR-Compound 30: (400 MHz, CD₃OD, δ ppm): δ1.40-1.53 (m, 4H), 1.83-1.92 (m, 9H), 2.05-2.25 (m, 6H), 2.35-2.37 (m, 2H), 2.66-3.05 (m, 14H), 3.32-3.33 (m, 4H), 3.29-3.32 (m, 1H), 3.42 (s, 2H), 3.53-3.56 (m, 5H), 4.29-4.31 (d, 1H), 5.08-5.13 (m, 1H), 7.10-7.14 (d, 2H), 7.29-7.31 (m, 1H), 7.48-7.58 (m, 3H), 7.64-7.69 (m, 3H), 8.35 (s, 1H), 8.44 (s, 1H).

### Example 29. Compound 31

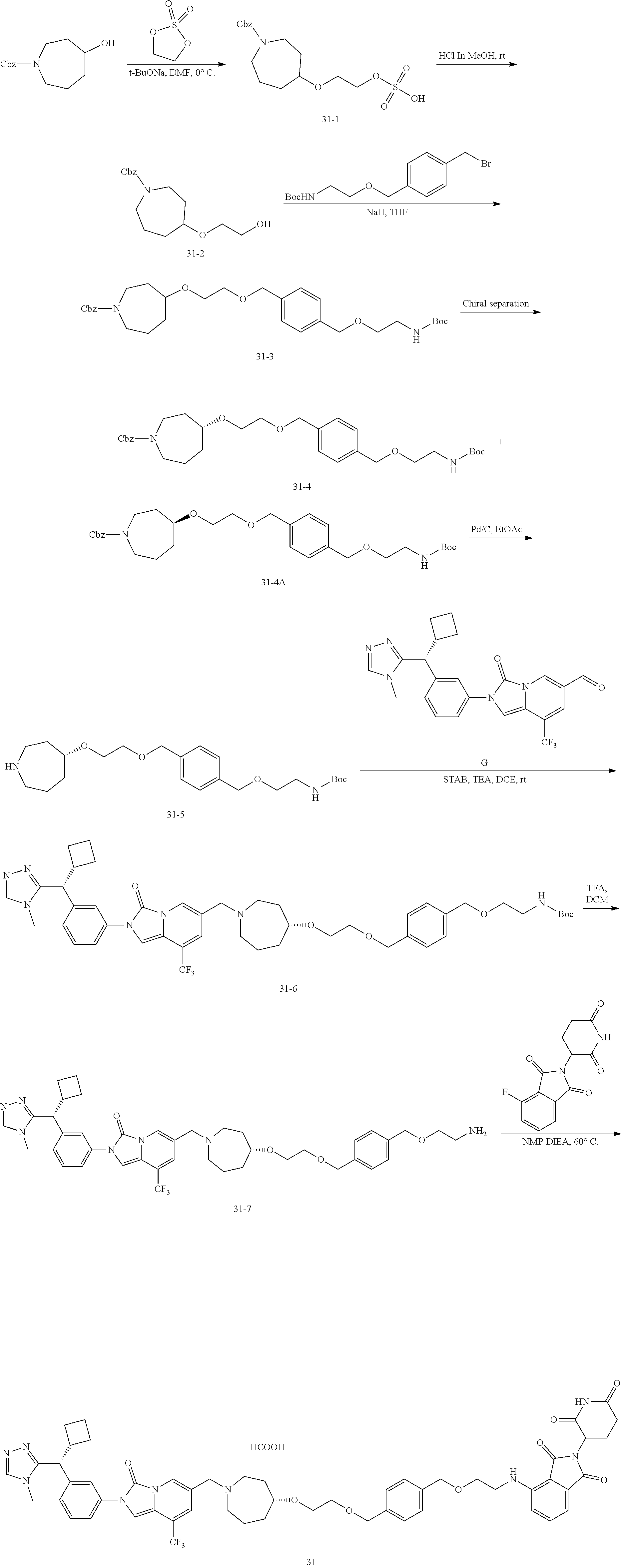

### Synthesis of Compound 31-1.

To a stirred mixture of benzyl 4-hydroxyazepane-1-carboxylate (10 g, 40.111 mmol, 1 equiv) and 1,3,2lambda6-dioxathiolane-2,2-dione (12.45 g, 100.277 mmol, 2.5 equiv) in DMF (100 mL) were added t-BuONa (9.64 g, 100.277 mmol, 2.5 equiv) in portions at 0° C. under air atmosphere. The resulting mixture was stirred for additional overnight at room temperature. The resulting mixture was diluted with water (400 mL). The aqueous layer was extracted with CH₂Cl₂ (3×150 mL). The resulting mixture was concentrated under reduced pressure. The crude product mixture was used in the next step directly without further purification. This resulted in Compound 31-1 (15 g, 15.02%) as a brown yellow oil.

### Synthesis of Compound 31-2.

To a stirred solution of Compound 31-1 (15 g, 40.169 mmol, 1 equiv) in MeOH (190 mL) was added CH₃COCl (27.00 mL, 378.344 mmol, 9.42 equiv) dropwise at 0° C. The resulting mixture was stirred for 3 h at room temperature. The mixture was acidified to pH 7 with saturated NaHCO₃ (aq.). The resulting mixture was extracted with 3×200 mL of MTBE. The resulting mixture was concentrated under reduced pressure. The residue was purified by silica gel column chromatography, eluted with PE/EA (1:1) to afford Compound 31-2 (600 mg, 5.09%) as a yellow oil.

### Synthesis of Compound 31-3.

To a solution of Compound 31-2 (13 g, 44.314 mmol, 1 equiv) in THF (40 mL) was added sodium hydride (2.13 g, 53.177 mmol, 1.2 equiv 60% in oil) at 0 degrees C. The mixture was stirred for 15 min. To the above mixture Compound 31-3 (22.88 g, 66.471 mmol, 1.5 equiv) was added. The resulting mixture was stirred for overnight at room temperature. The reaction mixture was quenched with sat. NH₄Cl (150 mL) and extracted with DCM (3×50 mL). The combined organic layers were concentrated under reduced pressure. The residue was purified by silica gel column chromatography, eluted with PE/EA (1:1). The crude product was purified by reverse flash chromatography with the following conditions: column, C18 silica gel; mobile phase, MeCN in Water (10 mmol/L NH₄HCO₃), 15% to 90% gradient in 30 min; UV 220 nm. This resulted in Compound 31-3 (7 g, 26.96%) as a colorless oil.

### Synthesis of Compound 31-4.

Compound 31-3 (3.5 g) was separated by Prep-CHIRAL-HPLC with the following conditions (Column: CHIRAL ART Amylose-C NEO, 5*25 cm, 5 μm; Mobile Phase A: CO₂, Mobile Phase B: MeOH (0.1% 2M NH₃-MeOH); Flow rate: 200 mL/min; Gradient: isocratic 60% B; Column Temperature (° C.): 35; Back Pressure (bar): 100; Wave Length: 220 nm; RT1 (min): 6.44; RT2 (min): 8.01; the first peak was product) to afford Compound 31-4 (1.1 g, 31.43%) (RT1 (min): 6.44) as a colorless oil and Compound 31-4A (1 g, 28.57%) (RT2 (min): 8.01) as a colorless oil.

### Synthesis of Compound 31-5.

To a solution of benzyl Compound 31-4 (1.15 g, 2.066 mmol, 1 equiv) in EtOAc (30 mL) was added Pd/C (10%, 0.3 g) under nitrogen atmosphere in a 100 mL round-bottom flask. The mixture was hydrogenated at room temperature for 2 h under hydrogen atmosphere using a hydrogen balloon, filtered through a Celite pad and concentrated under reduced pressure. This resulted in Compound 31-5 (850 mg, 94.82%) as a colorless oil.

### Synthesis of Compound 31-6.

To a stirred solution of Compound 31-5 (371.13 mg, 0.878 mmol, 1 equiv) and Intermediate G (400 mg, 0.878 mmol, 1.00 equiv) in DCE (10 mL) was added TEA (88.87 mg, 0.878 mmol, 1 equiv) and STAB (372.28 mg, 1.756 mmol, 2 equiv). The resulting mixture was stirred for overnight at room temperature under nitrogen atmosphere. The reaction was quenched by the addition of sat. NaHCO₃ (aq.) (15 mL). The resulting mixture was extracted with CH₂Cl₂/MeOH=10/1 (2×20 mL). The combined organic layers were concentrated under reduced pressure. The residue was purified by reverse flash chromatography with the following conditions: column, C18 silica gel; mobile phase, MeCN in water (10 mmol/L NH₄HCO₃), 30% to 100% gradient in 30 min; detector, UV 254 nm. This resulted in Compound 31-6 (450 mg, 59.44%) as a yellow solid.

### Synthesis of Compound 31-7.

To a stirred solution of Compound 31-6 (450 mg, 0.522 mmol, 1 equiv) in DCM (4 mL) was added TFA (1 mL). The resulting mixture was stirred for 2 h at room temperature. The resulting mixture was concentrated under reduced pressure. The residue was purified by Prep-TLC (CH₂Cl₂/MeOH 10:1) to afford Compound 31-7 (350 mg, 88.00%) as a yellow solid.

### Synthesis of Compound 31.

To a stirred solution of Compound 31-7 (170 mg, 0.223 mmol, 1 equiv) and 2-(2,6-dioxopiperidin-3-yl)-4-fluoroisoindole-1,3-dione (184.90 mg, 0.669 mmol, 3 equiv) in NMP (2 mL) was added DIEA (28.84 mg, 0.223 mmol, 1 equiv). The resulting mixture was stirred for overnight at 60° C. The reaction solution was purified by Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 28% B to 40% B in 7 min, 40% B; Wave Length: 254; 220 nm; RT1 (min): 6.62; Number Of Runs: 0) to afford Compound 31 (25 mg, 10.53%) as a yellow solid.

LCMS-Compound 31: (ES, m/z): [M−HCOOH+H]⁺ 1018

NMR-Compound 31: (400 MHz, CD₃OD, δ ppm): δ1.65-2.02 (m, 12H), 2.06-2.13 (m, 1H), 2.16-2.30 (m, 1H), 2.70-2.94 (m, 6H), 2.96-3.07 (m, 1H), 3.49-3.51 (m, 2H), 3.56 (s, 3H), 3.63-3.66 (m, 4H), 3.69-3.72 (m, 5H), 4.28-4.30 (d, 1H), 4.54-4.56 (d, 4H), 5.06-5.09 (m, 1H), 6.98-7.02 (m, 2H), 7.08 (s, 1H), 7.13 (s, 1H), 7.28-7.36 (m, 5H), 7.46-7.52 (m, 2H), 7.63-7.66 (m, 2H), 7.76 (s, 1H), 8.35 (s, 1H), 8.41 (s, 1H).

### Example 30. Compound 32

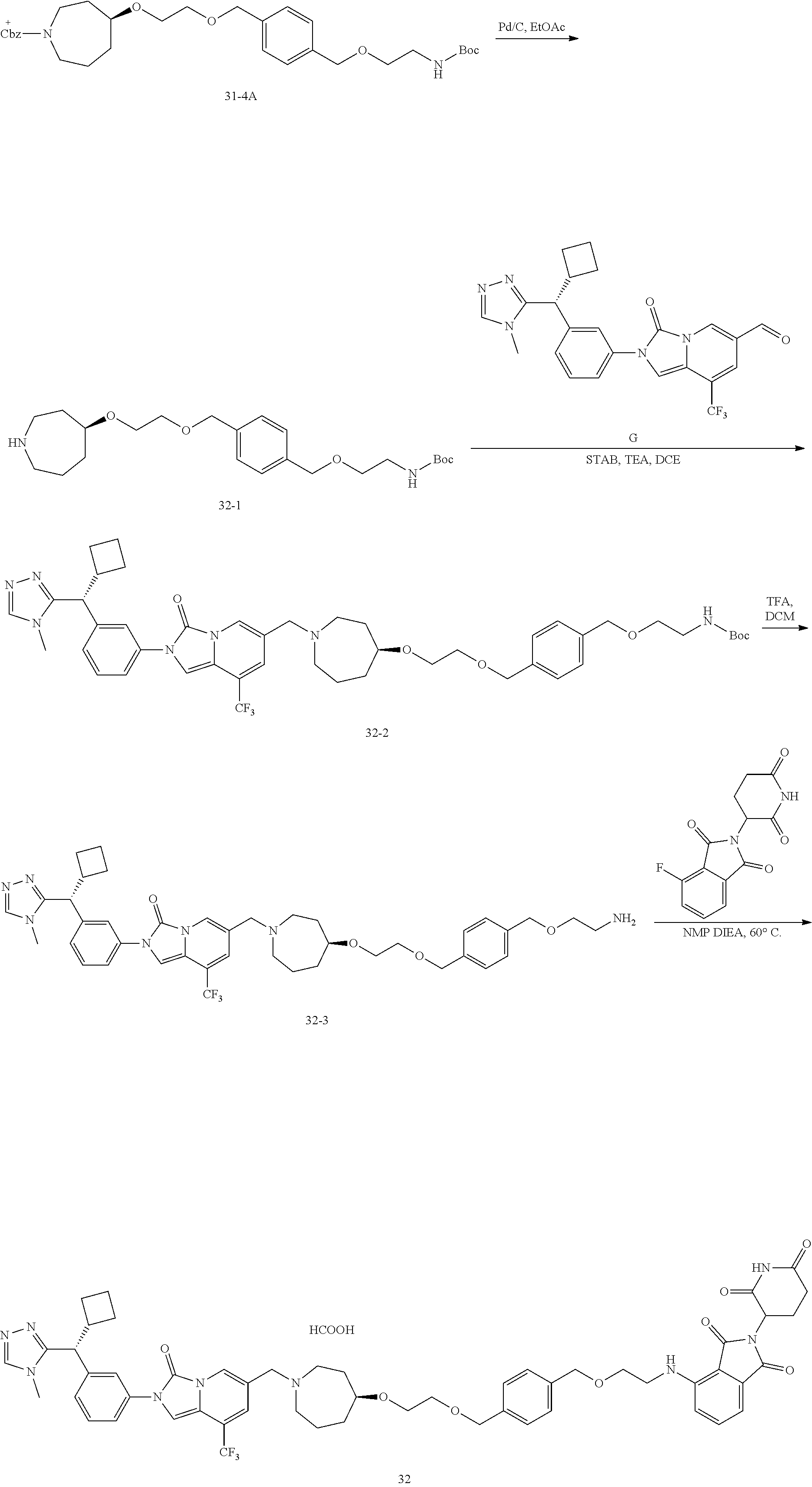

### Synthesis of Compound 32-1.

To a solution of Compound 31-4A (1 g, 1.796 mmol, 1 equiv) in EtOAc (30 mL) was added Pd/C (10%, 200 mg) under nitrogen atmosphere in a 100 mL round-bottom flask. The mixture was hydrogenated at room temperature for 2 h under hydrogen atmosphere using a hydrogen balloon, filtered through a Celite pad and concentrated under reduced pressure. This resulted in Compound 32-1 (750 mg, 98.81%) as a colorless oil.

### Synthesis of Compound 32-2.

To a stirred solution of Compound 32-1 (371.13 mg, 0.878 mmol, 1 equiv) and Intermediate G (400 mg, 0.878 mmol, 1.00 equiv) in DCE (10 mL) was added TEA (88.87 mg, 0.878 mmol, 1 equiv) and STAB (372.28 mg, 1.756 mmol, 2 equiv). The resulting mixture was stirred for overnight at room temperature under nitrogen atmosphere. The reaction was quenched by the addition of sat. NaHCO₃ (aq.) (15 mL). The resulting mixture was extracted with CH₂Cl₂/MeOH (2×20 mL). The combined organic layers were concentrated under reduced pressure. The residue was purified by reverse flash chromatography with the following conditions: column, C18 silica gel; mobile phase, MeCN in water (10 mmol/L NH₄HCO₃), 30% to 100% gradient in 30 min; detector, UV 254 nm. This resulted in Compound 32-2 (450 mg, 59.44%) as a yellow solid.

### Synthesis of Compound 32-3.

To a stirred solution of Compound 32-2 (420 mg, 0.487 mmol, 1 equiv) in DCM (4 mL) was added TFA (1 mL). The resulting mixture was stirred for 4 h at room temperature. The resulting mixture was concentrated under reduced pressure. The residue was purified by Prep-TLC (CH₂Cl₂/MeOH 10:1) to afford Compound 32-3 (280 mg, 75.43%) as a yellow solid.

### Synthesis of Compound 32.

To a stirred solution of Compound 32-3 (150 mg, 0.197 mmol, 1 equiv) and 2-(2,6-dioxopiperidin-3-yl)-4-fluoroisoindole-1,3-dione (163.15 mg, 0.591 mmol, 3 equiv) in NMP (2 mL) was added DIEA (25.45 mg, 0.197 mmol, 1 equiv). The resulting mixture was stirred for overnight at 60° C. The reaction solution was purified by Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 28% B to 40% B in 7 min, 40% B; Wave Length: 254; 220 nm; RT1 (min): 6.52) to afford Compound 32 (29.6 mg, 14.13%) as a yellow solid.

LCMS-Compound 32: (ES, m/z): [M−HCOOH+H]⁺ 1018

NMR-Compound 32: (400 MHz, CD₃OD, δ ppm): δ 1.65-2.02 (m, 12H), 2.06-2.13 (m, 1H, 2.16-2.30 (m, 1H), 2.70-2.86 (m, 6H), 2.87-2.96 (m, 6H), 2.98-3.07 (m, 1H), 3.49-3.51 (m, 2H), 3.56 (s, 3H), 3.60-3.66 (m, 4H), 3.69-3.72 (m, 5H), 4.29-4.32 (d, 1H), 4.54-4.56 (d, 4H), 5.06-5.11 (m, 1H), 6.99-7.02 (m, 2H), 7.08 (s, 1H), 7.13 (s, 1H), 7.28-7.37 (m, 5H), 7.46-7.52 (m, 2H), 7.63-7.65 (m, 2H), 7.76 (s, 1H), 8.36 (s, 1H).

### Example 31. Compound 33

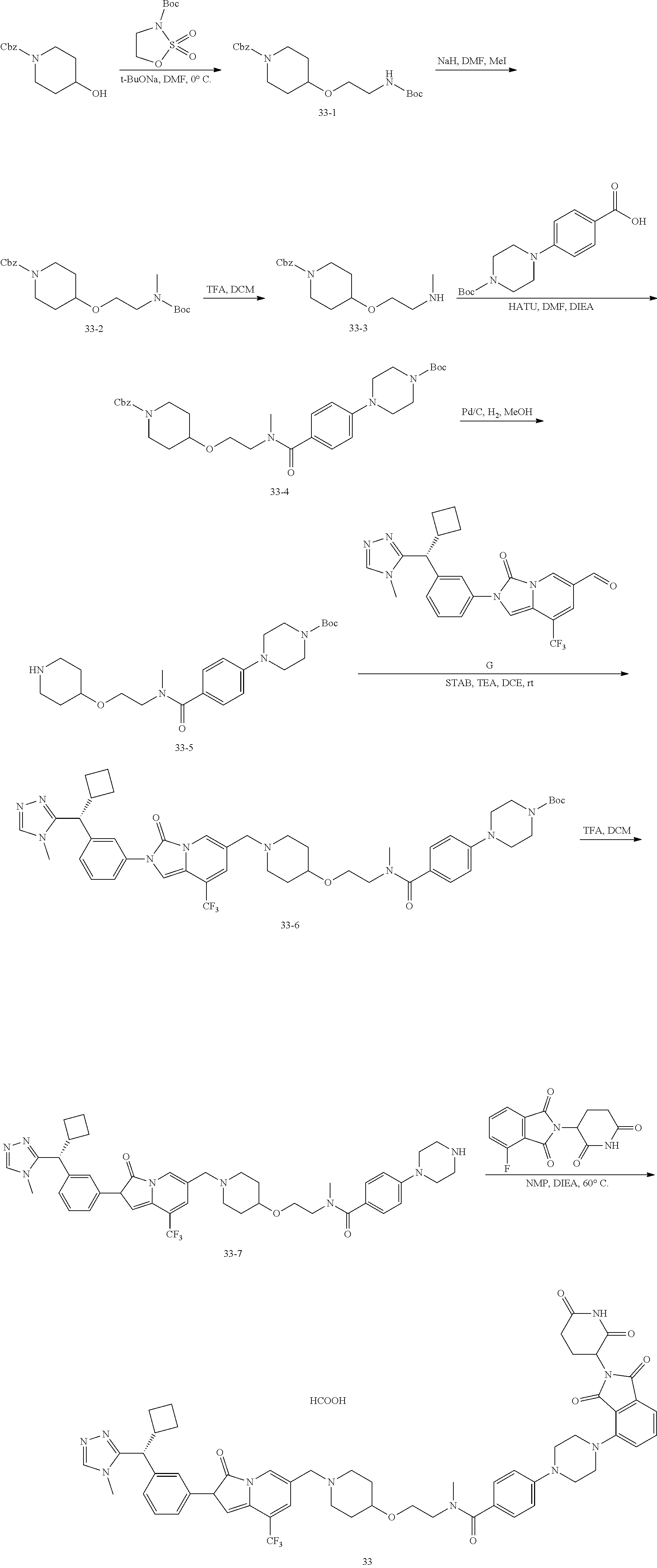

### Synthesis of Compound 33-1.

Into a 1000 mL 3-necked round-bottom flask were added benzyl 4-hydroxypiperidine-1-carboxylate (10 g, 42.502 mmol, 1 equiv), DMF (500 mL) and tert-butyl 2,2-dioxo-1,2lambda6,3-oxathiazolidine-3-carboxylate (18.03 g, 80.754 mmol, 1.9 equiv) at room temperature. To the above mixture was added t-BuONa (8.17 g, 85.004 mmol, 2 equiv) in portions over 10 min at 0° C. The resulting mixture was stirred for 4 h at 0 degrees under nitrogen atmosphere. The reaction was quenched by the addition of sat. NH₄Cl (aq.) (300 mL) at room temperature. The resulting mixture was extracted with EtOAc (3×200 mL). The combined organic layers were washed with water (200 mL), dried over anhydrous Na₂SO₄. After filtration, the filtrate was concentrated under reduced pressure. The resulting mixture was concentrated under vacuum. This resulted in Compound 33-1 (3.8 g, 22.44%) as a yellow oil.

### Synthesis of Compound 33-2.

A solution of Compound 33-1 (2.1 g, 5.549 mmol, 1 equiv) in DMF (21 mL) was treated with NaH (0.45 g, 11.098 mmol, 2 equiv, 60% in oil) for 1 h at 0° C. under nitrogen atmosphere followed by the addition of MeI (1.58 g, 11.098 mmol, 2 equiv) dropwise at room temperature in a 100 mL 3-necked round-bottom flask. The resulting mixture was stirred for overnight at room temperature under nitrogen atmosphere. The reaction was quenched by the addition of sat. NH₄Cl (aq.) (20 mL) at room temperature. The resulting mixture was extracted with EtOAc (3×20 mL). After filtration, the filtrate was concentrated under reduced pressure. The residue was purified by silica gel column chromatography, eluted with PE/EA (1:1) to afford Compound 33-2 (1.66 g, 72.41%) as a white solid.

### Synthesis of Compound 33-3.

Into a 50 mL round-bottom flask were added Compound 33-2 (1.64 g, 4.178 mmol, 1 equiv), TFA (0.95 g, 8.356 mmol, 2 equiv) and DCM (16.4 mL) at room temperature. The resulting mixture was stirred for overnight at room temperature. The resulting mixture was concentrated under vacuum. This resulted in Compound 33-3 (1.13 g, 85.10%) as a white solid.

### Synthesis of Compound 33-4.

Into a 25 mL 3-necked round-bottom flask were added Compound 33-3 (1.13 g, 3.865 mmol, 1 equiv), HATU (2.20 g, 5.798 mmol, 1.5 equiv), DMF (11.3 mL) and DIEA (2.00 g, 15.460 mmol, 4 equiv) at room temperature. The resulting mixture was stirred for 2 h at room temperature. To the above mixture was added 4-[4-(tert-butoxycarbonyl)piperazin-1-yl]benzoic acid (1.18 g, 3.865 mmol, 1 equiv) at room temperature. The resulting mixture was stirred for additional overnight at room temperature. The reaction was quenched with water at room temperature. The precipitated solids were collected by filtration and washed with water (3×10 mL). This resulted in Compound 33-4 (1.3 g, 52.13%) as a white solid.

### Synthesis of Compound 33-5.

To a solution of Compound 33-4 (480 mg, 0.827 mmol, 1 equiv) in 9.6 mL MeOH was added Pd/C (57.18 mg) under nitrogen atmosphere in a 25 mL round-bottom flask. The mixture was hydrogenated at room temperature for overnight under hydrogen atmosphere using a hydrogen balloon, filtered through a Celite pad and concentrated under reduced pressure. This resulted in Compound 33-5 (353 mg, 86.07%) as a white solid.

### Synthesis of Compound 33-6.

To a stirred solution of Compound 33-5 (353.01 mg, 0.790 mmol, 1 equiv) and Intermediate G (360 mg, 0.790 mmol, 1.00 equiv) in DCE (10 mL) was added TEA (79.99 mg, 0.790 mmol, 1 equiv) and STAB (335.05 mg, 1.580 mmol, 2 equiv). The resulting mixture was stirred for overnight at room temperature. The reaction was quenched by the addition of sat. NaHCO₃ (aq.) (15 mL). The resulting mixture was extracted with CH₂Cl₂/MeOH=10/1 (2×15 mL). The combined organic layers were concentrated under reduced pressure. The residue was purified by reverse flash chromatography with the following conditions: column, C18 silica gel; mobile phase, MeCN in water (10 mmol/L NH₄HCO₃), 15% to 85% gradient in 30 min; detector, UV 254 nm. This resulted in Compound 33-6 (380 mg, 54.26%) as a yellow solid.

### Synthesis of Compound 33-7.

To a stirred solution of Compound 33-6 (380 mg, 0.429 mmol, 1 equiv) in DCM (4 mL) was added TFA (1 mL) under nitrogen atmosphere. The resulting mixture was stirred for 4 h at room temperature under nitrogen atmosphere. The resulting mixture was concentrated under reduced pressure. The residue was purified by Prep-TLC (CH₂Cl₂/MeOH 10:1) to afford Compound 33-7 (210 mg, 62.30%) as a yellow solid.

### Synthesis of Compound 33.

To a stirred solution of Compound 33-7 (150 mg, 0.191 mmol, 1 equiv) and 2-(2,6-dioxopiperidin-3-yl)-4-fluoroisoindole-1,3-dione (158.16 mg, 0.573 mmol, 3 equiv) in NMP (2 mL) was added DIEA (24.67 mg, 0.191 mmol, 1 equiv). The resulting mixture was stirred for overnight at 60° C. The reaction solution was purified by Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 24% B to 35% B in 8 min, 35% B; Wave Length: 254; 220 nm; RT1 (min): 7.77) to afford Compound 33 (18.1 mg, 8.72%) as a yellow solid.

LCMS-Compound 33: (ES, m/z): [M−HCOOH+H]⁺ 1042

NMR-Compound 33: (400 MHz, CD₃OD, δ ppm): δ1.74-1.77 (m, 4H), 1.79-1.89 (m, 6H), 1.90-1.92 (m, 1H), 1.95-1.96 (m, 1H), 2.42-2.51 (m, 2H), 2.72-2.77 (m, 2H), 2.85-2.89 (m, 3H), 3.13-3.15 (m, 3H), 3.50-3.69 (m, 19H), 4.27-4.30 (d, 1H), 5.10-5.13 (m, 1H), 7.07-7.15 (m, 3H), 7.27-7.29 (m, 1H), 7.37-7.43 (m, 4H), 7.47-7.51 (m, 1H), 7.64-7.66 (m, 2H), 7.69-7.73 (m, 1H), 7.77 (s, 1H), 8.36 (s, 2H).

### Example 32. Compound 34

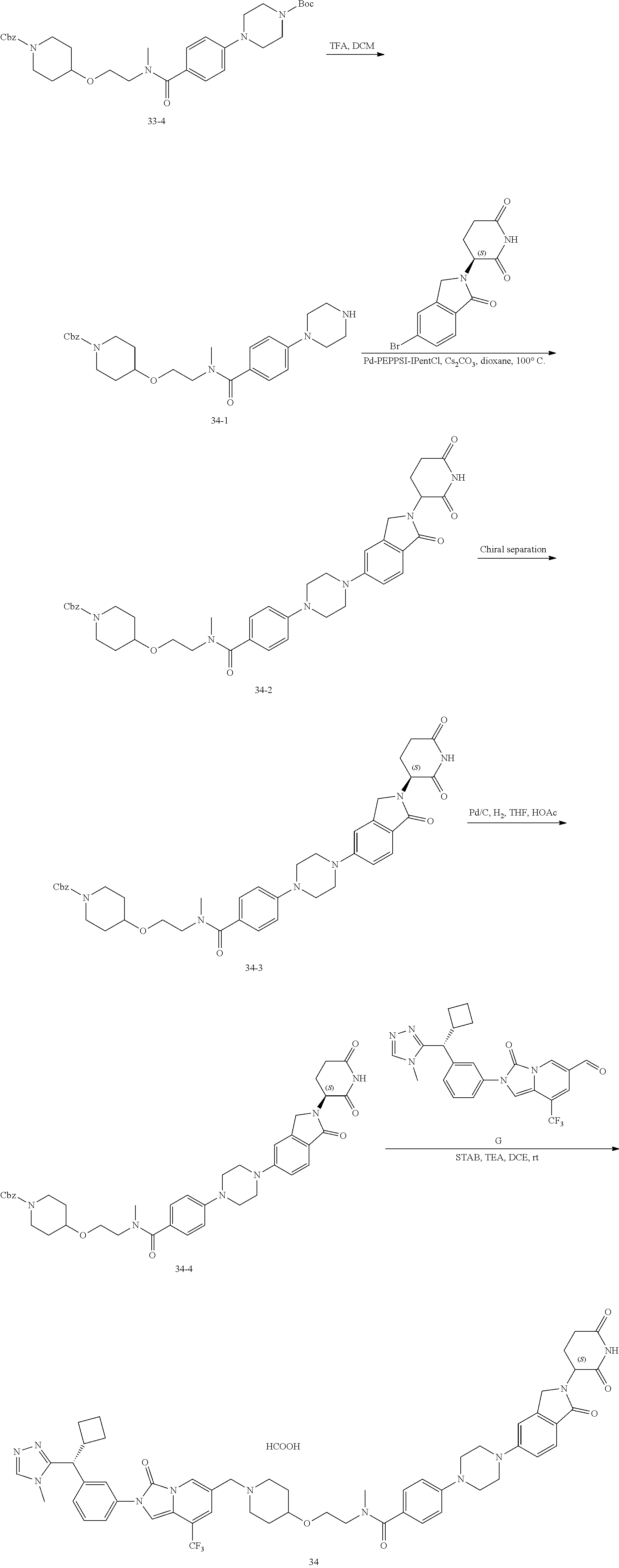

### Synthesis of Compound 34-1.

Into a 25 mL round-bottom flask were added Compound 33-4 (800 mg, 1.378 mmol, 1 equiv), TFA (314.15 mg, 2.756 mmol, 2 equiv) and DCM (8 mL) at room temperature. The resulting mixture was stirred for overnight at room temperature. The resulting mixture was concentrated under vacuum. This resulted in Compound 34-1 (540 mg, 79.11%) as a white solid.

### Synthesis of Compound 34-2.

To a stirred mixture of Compound 34-1 (510 mg, 1.061 mmol, 1 equiv) and (3S)-3-(5-bromo-1-oxo-3H-isoindol-2-yl)piperidine-2,6-dione (411.49 mg, 1.273 mmol, 1.2 equiv) in dioxane (10 mL) were added Cs₂CO₃ (1037.23 mg, 3.183 mmol, 3 equiv) and Pd-PEPPSI-IPentCl₂-methylpyridine (o-picoline (89.26 mg, 0.106 mmol, 0.1 equiv) at room temperature under nitrogen atmosphere. The resulting mixture was stirred for overnight at 100° C. under nitrogen atmosphere. The reaction was quenched by the addition of Water/HOAc=10/1 (30 mL). The resulting mixture was extracted with CH₂Cl₂/MeOH=5/1 (3×50 mL). The combined organic layers were concentrated under reduced pressure. The residue was purified by silica gel column chromatography, eluted with CH₂Cl₂/MeOH (10:1) to afford Compound 34-2 (500 mg, 65.19%) as a grey solid.

### Synthesis of Compound 34-3.

Compound 34-2 (500 mg) was purified by Prep-Chiral-SFC with the following conditions (Column: CHIRALPAK IH, 3*25 cm, 5 μm; Mobile Phase A: CO₂, Mobile Phase B: MeOH:DCM=1:1; Flow rate: 100 mL/min; Gradient: isocratic 50% B; Column Temperature (° C.): 35; Back Pressure (bar): 100; Wave Length: 220 nm; RT1 (min): 2.63; RT2 (min): 4.93; The second peak was product) to afford Compound 34-3 (160 mg, 32.00%) as a white solid.

### Synthesis of Compound 34-4.

To a solution of Compound 34-3 (160 mg, 0.221 mmol, 1 equiv) in EtOAc (10 mL) and HOAc (1 mL) was added Pd/C (30 mg, 10%) under nitrogen atmosphere in a 100 mL round-bottom flask. The mixture was hydrogenated at room temperature for overnight under hydrogen atmosphere using a hydrogen balloon, filtered through a Celite pad and concentrated under reduced pressure. This resulted in Compound 34-4 (130 mg, 99.76%) as a white solid.

### Synthesis of Compound 34.

To a stirred solution of Compound 34-4 (130 mg, 0.221 mmol, 1 equiv) and Intermediate G (150.86 mg, 0.332 mmol, 1.5 equiv) in DCE (10 mL) were added Et₃N (22.35 mg, 0.221 mmol, 1 equiv) and NaBH(OAc)₃ (93.60 mg, 0.442 mmol, 2 equiv). The resulting mixture was stirred for overnight at room temperature. The resulting mixture was diluted with water/HOAc=10/1 (20 mL). The resulting mixture was extracted with CH₂Cl₂/MeOH (4×20 mL). The combined organic layers were concentrated under reduced pressure. The residue was purified by Prep-HPLC with the following conditions (Column: XSelect CSH Fluoro Phenyl, 30*150 mm, 5 μm; Mobile Phase A: Water (0.1% FA), Mobile Phase B: ACN; Flow rate: 60 mL/min; Gradient: 22% B to 33% B in 7 min, 33% B; Wave Length: 254; 220 nm; RT1 (min): 6.27) to afford Compound 34 (22.1 mg, 9.32%) as a yellow solid.

LCMS-Compound 34: (ES, m/z): [M−HCOOH+H]⁺ 1028; [M−HCOOH+Na]⁺ 1050

NMR-Compound 34: (400 MHz, DMSO, δ ppm): δ1.74-1.92 (m, 10H), 2.10-2.17 (m, 1H), 2.18-2.25 (m, 1H), 2.42-2.51 (m, 1H), 2.53-2.64 (m, 2H), 2.80-2.82 (m, 1H), 2.87-2.95 (m, 2H), 3.11-3.15 (m, 3H), 3.46-3.69 (m, 20H), 4.28-4.30 (d, 1H), 4.41-4.43 (m, 2H), 5.09-5.14 (m, 1H), 7.06-7.15 (m, 6H), 7.28-7.30 (m, 1H), 7.40-7.43 (m, 2H), 7.50-7.52 (t, 1H), 7.64-7.68 (m, 3H), 7.77 (s, 1H), 8.36 (s, 2H).

### Example 33. Compound 35

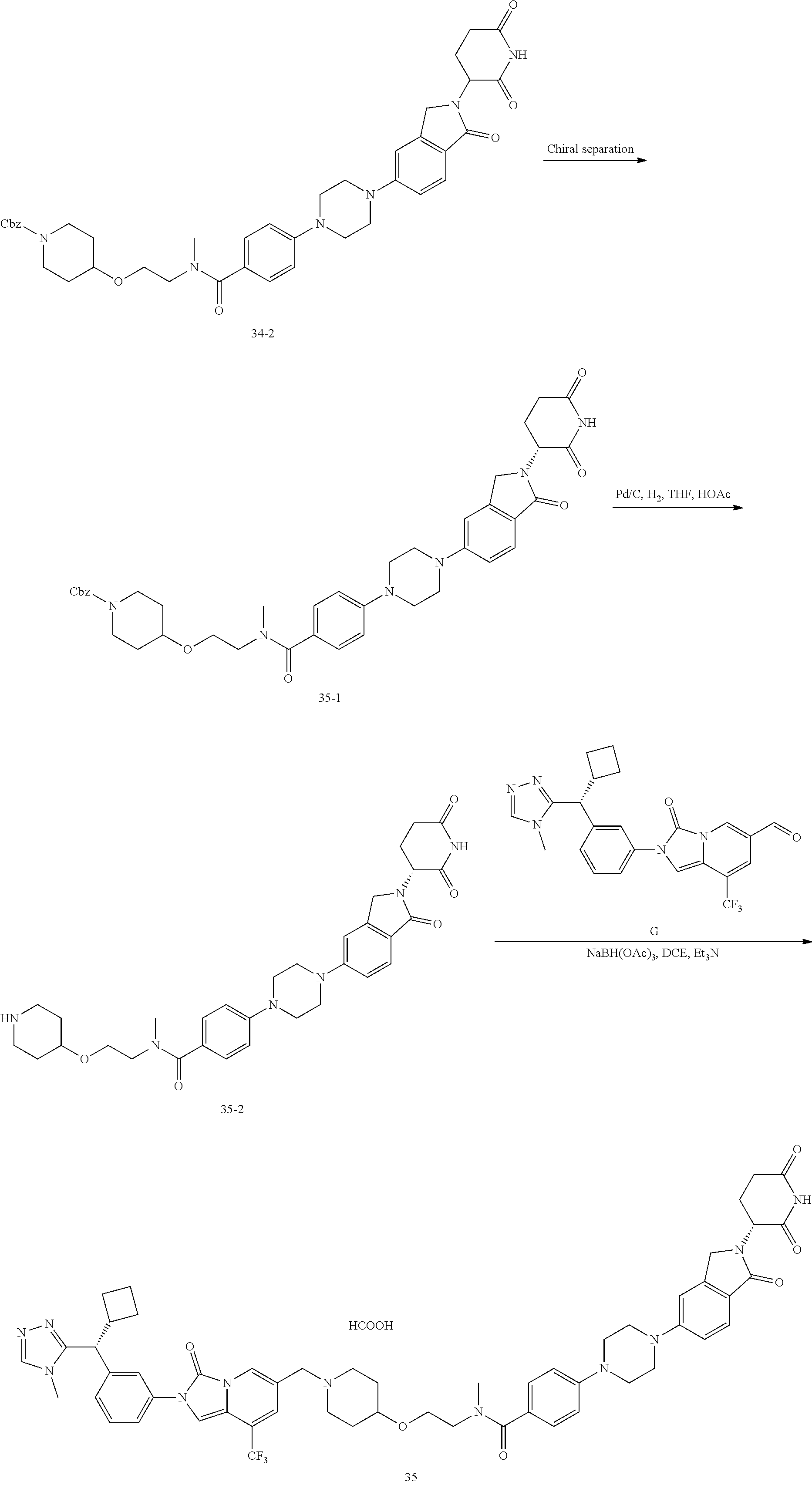 [SMILES: CN(CCOC1CCN(C(=O)OCc2ccccc2)CC1)C(=O)c1ccc(N2CCN(c3ccc4c(c3)CN(C3CCC(=O)NC3=O)C4=O)CC2)cc1 ; CN(CCOC1CCN(C(=O)OCc2ccccc2)CC1)C(=O)c1ccc(N2CCN(c3ccc4c(c3)CN([C@@H]3CCC(=O)NC3=O)C4=O)CC2)cc1 ; CN(CCOC1CCNCC1)C(=O)c1ccc(N2CCN(c3ccc4c(c3)CN([C@@H]3CCC(=O)NC3=O)C4=O)CC2)cc1 ; Cn1cnnc1[C@@H](c1cccc(-n2cc3c(C(F)(F)F)cc(C=O)cn3c2=O)c1)C1CCC1 ; CN(CCOC1CCN(Cc2cc(C(F)(F)F)c3cn(-c4cccc([C@H](c5nncn5C)C5CCC5)c4)c(=O)n3c2)CC1)C(=O)c1ccc(N2CCN(c3ccc4c(c3)CN([C@@H]3CCC(=O)NC3=O)C4=O)CC2)cc1 ; "HCOOH"]

### Synthesis of Compound 35-1.

Compound 34-2 (500 mg) was separated by Prep-Chiral-SFC with the following conditions (Column: CHIRALPAK IH, 3*25 cm, 5 μm; Mobile Phase A: CO₂, Mobile Phase B: MeOH:DCM=1:1; Flow rate: 100 mL/min; Gradient: isocratic 50% B; Column Temperature (° C.): 35; Back Pressure (bar): 100; Wave Length: 220 nm; RT1 (min): 2.63; RT2 (min): 4.93; The first peak was product) to afford Compound 35-1 (180 mg, 36.00%) as a grey solid.

### Synthesis of Compound 35-2.

To a solution of Compound 35-1 (180 mg, 0.249 mmol, 1 equiv) in EtOAc (10 mL) and HOAc (1 mL) was added Pd/C (30 mg, 10%) under nitrogen atmosphere in a 100 mL round-bottom flask. The mixture was hydrogenated at room temperature for overnight under hydrogen atmosphere using a hydrogen balloon, filtered through a Celite pad and concentrated under reduced pressure. This resulted in Compound 35-2 (120 mg, 81.86%) as a white solid.

### Synthesis of Compound 35.

To a stirred solution of Compound 35-2 (120 mg, 0.204 mmol, 1 equiv) and Compound Intermediate G (139.25 mg, 0.306 mmol, 1.5 equiv) in DCE (10 mL) were added Et₃N (20.63 mg, 0.204 mmol, 1 equiv) and NaBH(OAc)₃ (86.40 mg, 0.408 mmol, 2 equiv). The resulting mixture was stirred for overnight at room temperature. The resulting mixture was diluted with water/HOAc=10/1 (20 mL). The resulting mixture was extracted with CH₂Cl₂/MeOH=10/1 (3×30 mL). The combined organic layers were concentrated under reduced pressure. The residue was purified by Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 22% B to 33% B in 7 min, 33% B; Wave Length: 254; 220 nm; RT1 (min): 6.21) to afford Compound 35 (15.8 mg, 7.22%) as a yellow solid.

LCMS-Compound 34: (ES, m/z): [M−HCOOH+H]⁺ 1028: [M−HCOOH+Na]⁺ 1050

NMR-Compound 34: (400 MHz, CD₃OD, δ ppm): δ1.74-1.92 (m, 11H), 2.10-2.26 (m, 3H), 2.42-2.51 (m, 2H), 2.53-2.64 (m, 2H), 2.80-2.82 (m, 2H), 2.87-2.93 (m, 3H), 3.11-3.15 (m, 4H), 3.46-3.69 (m, 23H), 4.28-4.30 (d, 1H), 4.42-4.44 (m, 2H), 5.10-5.15 (m, 1H), 7.06-7.15 (m, 7H), 7.28-7.30 (m, 1H), 7.40-7.43 (m, 3H), 7.48-7.52 (t, 1H), 7.64-7.68 (m, 3H), 7.76 (s, 1H), 8.32 (s, 1H), 8.36 (s, 1H).

### Example 34. Compound 36

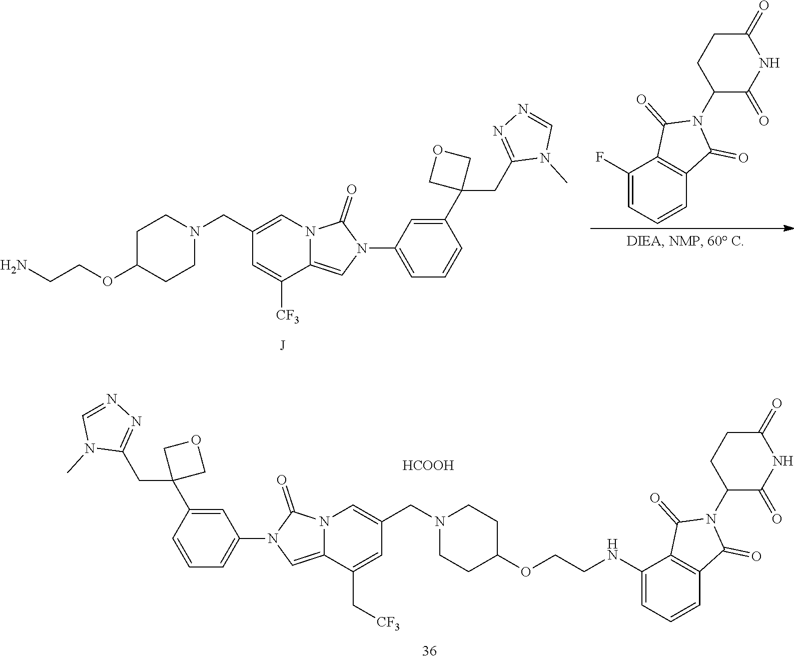

### Synthesis of Compound 36.

To a stirred solution of Intermediate J (300 mg, 0.51 mmol, 1.0 equiv) and 2-(2,6-dioxopiperidin-3-yl)-4-fluoroisoindole-1,3-dione (424 mg, 1.53 mmol, 3.0 equiv) in NMP (2 mL) was added DIEA (99 mg, 0.76 mmol, 1.5 equiv) at room temperature. The resulting mixture was stirred for overnight at 60° C. The reaction solution was purified by Prep-HPLC with the following conditions (Column: Xselect CSH C18 OBD Column 30*150 mm 5 μm, n; Mobile Phase A: water (0.1% FA), Mobile Phase B: ACN; Flow rate: 60 mL/min; Gradient: 16% B to 28% B in 9 min, 28% B; Wave Length: 254; 220 nm; RT1 (min): 8.26) to afford Compound 36 (28 mg, 6%) as a yellow solid.

LC-MS-Compound 36: (ES, m/z): [(M−HCOOH)/2+H]⁺ 421

H-NMR-Compound 36: (400 MHz, CD₃OD, δ ppm): 1.87-1.99 (m, 4H), 2.12-2.18 (m, 1H), 2.62 (s, 2H), 2.73-2.91 (4, 3H), 2.95 (s, 3H), 3.53-3.62 (m, 5H), 3.3.69 (s, 1H), 3.72-3.73 (d, 2H), 3.75 (s, 2H), 5.09-5.17 (m, 4H), 5.27-5.28 (m, 1H), 6.89-6.91 (d, 1H), 7.09-7.15 (m, 4H), 7.33 (s, 1H), 7.45-7.49 (t, 1H), 7.57-7.64 (m, 2H), 7.75 (s, 1H), 8.21-8.51 (m, 1H).

### Example 35. Compound 37

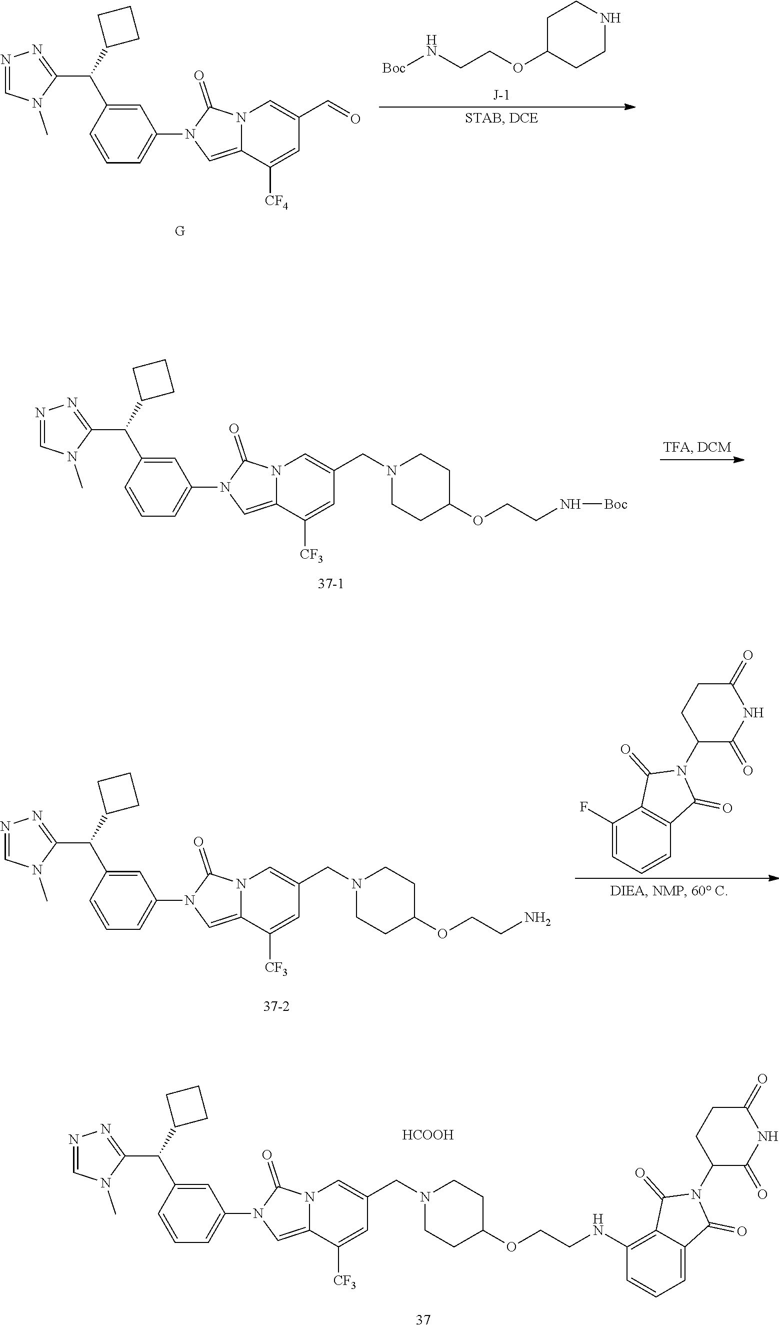

### Synthesis of Compound 37-1.

To a stirred solution of Intermediate G (1 g, 2.19 mmol, 1.0 equiv) and Intermediate J-1 (1.1 g, 4.39 mmol, 2.0 equiv) in DCE (10 mL) was added STAB (1.4 g, 6.58 mmol, 3.0 equiv) at room temperature. The resulting mixture was stirred for overnight at room temperature. The reaction was quenched by the addition of water (50 mL) at room temperature. The aqueous layer was extracted with CH₂Cl₂ (3×50 mL). The resulting mixture was concentrated under reduced pressure. The residue was purified by Prep-TLC (CH₂Cl₂/MeOH=10:1) to afford Compound 37-1 (1 g, 67%) as a yellow solid.

### Synthesis of Compound 37-2.

A solution of Compound 37-1 (1 g, 1.46 mmol, 1.0 equiv) and TFA (2.5 mL) in DCM (7.5 mL) was stirred for 3 h at room temperature. The mixture was neutralized to pH 7 with NH₃ in MeOH. The resulting mixture was concentrated under reduced pressure. The residue was purified by Prep-TLC (CH₂Cl₂/MeOH=10:1) to afford Compound 37-2 (600 mg, 70%) as a yellow solid.

### Synthesis of Compound 37.

To a stirred solution of Compound 37-2 (600 mg, 1.02 mmol, 1.0 equiv) and 2-(2,6-dioxopiperidin-3-yl)-4-fluoroisoindole-1,3-dione (851 mg, 3.08 mmol, 3.0 equiv) in NMP (4 mL) was added DIEA (199 mg, 1.54 mmol, 1.5 equiv) at room temperature. The resulting mixture was stirred for overnight at 60° C. under nitrogen atmosphere. The reaction solution was purified by Prep-HPLC with the following conditions (Column: XSelect CSH Fluoro Phenyl, 30*150 mm, 5 μm; Mobile Phase A: water (0.1% FA), Mobile Phase B: ACN; Flow rate: 60 mL/min; Gradient: 19% B to 29% B in 8 min, 29% B; Wave Length: 254; 220 nm; RT1 (min): 7.98) to afford Compound 37 (40.9 mg, 4.45%) as a yellow solid.

LC-MS-Compound 37: (ES, m/z): [(M−HCOOH)/2+H]⁺ 420

H-NMR-Compound 37: (400 MHz, CD₃OD, δ ppm): 1.79-1.93 (m, 9H), 2.15-2.27 (m, 3H), 2.67-2.90 (m, 7H), 3.49-3.90 (m, 8H), 3.72-3.75 (t, 2H), 4.29-4.32 (d, 1H), 5.15-5.16 (m, 1H), 7.09-7.15 (m, 4H), 7.27-7.29 (d, 1H), 7.48-7.52 (t, 1H), 7.59-7.61 (d, 1H), 7.64-7.69 (m, 2H), 7.77 (s, 1H), 8.37 (s, 1H), 8.42 (s, 1H).

### Example 36. Compound 38

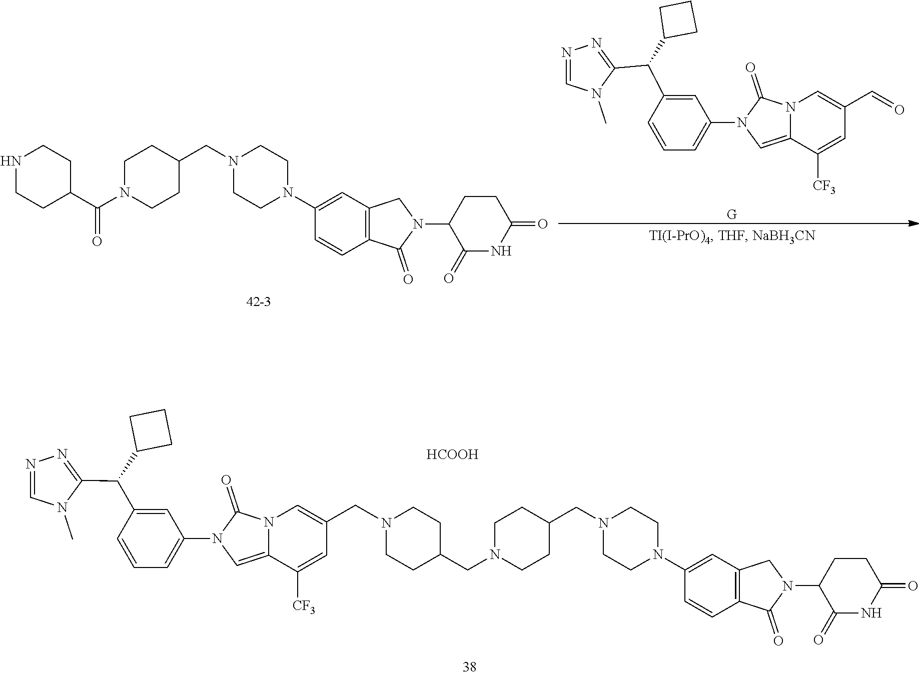

### Synthesis of Compound 38.

To a stirred solution of Compound 42-3 (300 mg, 0.57 mmol, 1.0 equiv) and Intermediate G (392 mg, 0.86 mmol, 1.5 equiv) in THF (3 mL) was added Ti(Oi-Pr)₄ (326 mg, 1.14 mmol, 2.0 equiv) at room temperature. The resulting mixture was stirred for overnight at room temperature. To the above mixture was added NaBH₃CN (54 mg, 0.86 mmol, 1.5 equiv) at room temperature. The resulting mixture was stirred for additional 2 h at room temperature. The reaction was quenched by the addition of 10% AcOH (aq.) (100 mL) at room temperature. The resulting mixture was concentrated under reduced pressure. The crude product (20 mg) was purified by Prep-HPLC with the following conditions (Column: Xselect CSH C18 OBD Column 30*150 mm 5 μm, n; Mobile Phase A: water (0.1% FA), Mobile Phase B: ACN; Flow rate: 60 mL/min; Gradient: 6% B to 18% B in 9 min, 18% B; Wave Length: 254; 220 nm; RT1 (min): 8.3; Number of Runs: 0) to afford Compound 38 (38 mg, 6%) as a yellow solid.

LC-MS-Compound 38: (ES, m/z): [(M−HCOOH)/2+H]⁺ 481

H-NMR-Compound 38: (400 MHz, CD₃OD, δ ppm): 1.35-1.44 (m, 5H), 1.84-1.87 (m, 1H), 1.91-1.94 (m, 8H), 2.07-2.10 (m, 2H), 2.18-2.26 (m, 4H), 2.37-2.38 (m, 2H), 2.51-2.56 (m, 2H), 2.66 (s, 4H), 2.80-2.81 (m, 1H), 2.92-3.07 (m, 8H), 3.39 (s, 3H), 3.44 (s, 1H), 3.51-3.59 (m, 5H), 4.28-4.31 (d, 1H), 4.41-4.42 (m, 2H), 5.09-5.14 (m, 1H), 7.09-7.14 (m, 4H), 7.29-7.53 (m, 3H), 7.64-7.70 (m, 3H), 8.29 (s, 1H), 8.37 (s, 1H).

### Example 37. Compound 39

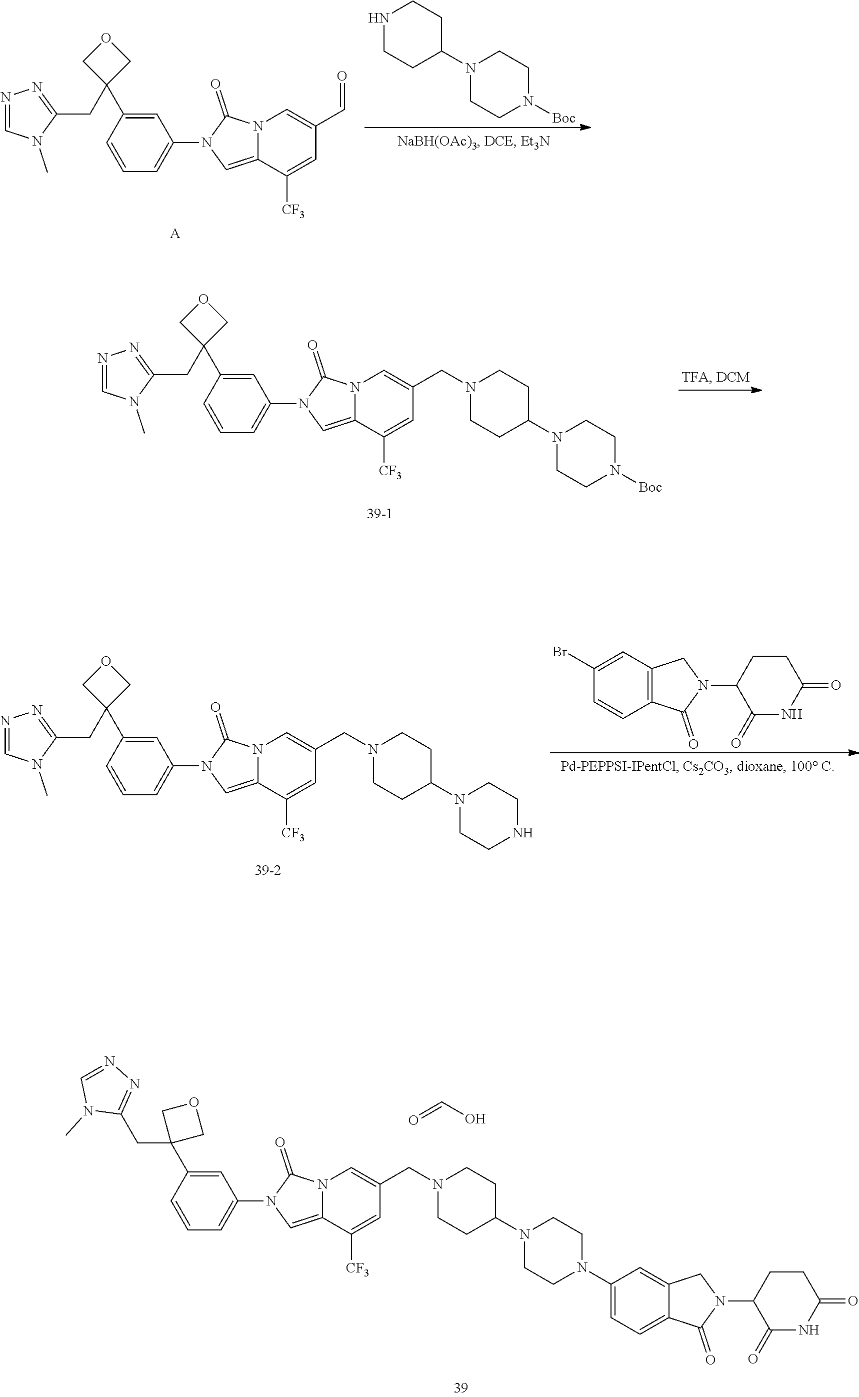

### Synthesis of Compound 39-1.

To a stirred solution of Intermediate A (1 g, 2.186 mmol, 1 equiv) and tert-butyl 4-(piperidin-4-yl)piperazine-1-carboxylate (1.18 g, 4.372 mmol, 2 equiv) in DCE (20 mL) were added Et₃N (0.22 g, 2.186 mmol, 1 equiv) and NaBH(OAc)₃ (0.93 g, 4.372 mmol, 2 equiv). The resulting mixture was stirred for overnight at room temperature under nitrogen atmosphere. The reaction was quenched by the addition of sat. NaHCO₃ (aq.) (30 mL). The resulting mixture was extracted with CH₂Cl₂/MeOH=10/1 (3×30 mL). The combined organic layers were concentrated under reduced pressure. The residue was purified by reverse lash chromatography with the following conditions: column, C18 silica gel; mobile phase, MeCN in Water (10 mmol/L NH₄HCO₃), 25% to 90% gradient in 30 min; detector, UV 254 nm. This resulted in Compound 39-1 (860 mg, 55.34%) as a yellow solid.

### Synthesis of Compound 39-2.

To a stirred solution of Compound 39-1 (850 mg, 1.196 mmol, 1 equiv) in DCM (10 mL) was added TFA (2 mL). The resulting mixture was stirred for 4 h at room temperature. The resulting mixture was concentrated under reduced pressure. The residue was purified by Prep-TLC (CH₂Cl₂/MeOH 10:1) to afford Compound 39-2 (610 mg, 83.53%) as a yellow solid.

### Synthesis of Compound 39.

To a stirred mixture of Compound 39-2 (624.40 mg, 1.932 mmol, 2 equiv) in dioxane (15 mL) were added Cs₂CO₃ (944.35 mg, 2.898 mmol, 3 equiv) and Pd-PEPPSI-IPentCl 2-methylpyridine (o-picoline) (81.27 mg, 0.097 mmol, 0.1 equiv) under nitrogen atmosphere. The resulting mixture was stirred for overnight at 100° C. under nitrogen atmosphere. The reaction was quenched by the addition of Water/HOAc=10/1 (20 mL). The resulting mixture was extracted with EtOAc (3×20 mL). The aqueous layer was concentrated under reduced pressure. The residue was purified by Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 6% B to 18% B in 7 min, 18% B; Wave Length: 254; 220 nm; RT1 (min): 6.98) to afford Compound 39 (65.9 mg, 7.59%) as a yellow solid.

LCMS-Compound 39: (ES, m/z): [M+H]⁺ 853;

NMR-Compound 39: (400 MHz, CD₃OD, δ ppm): δ1.78-1.84 (m, 2H), 2.13-2.18 (m, 4H), 2.33-2.38 (m, 2H), 2.46-2.50 (m, 1H), 2.77-2.81 (m, 1H), 2.87-2.95 (m, 2H), 2.98 (s, 3H), 3.17-3.23 (m, 6H), 3.53-3.55 (m, 6H), 3.68 (s, 2H), 4.38-4.48 (m, 2H), 5.07-5.15 (m, 5H), 6.94-6.96 (d, 2H), 7.11-7.16 (m, 4H), 7.30 (s, 1H), 7.47-7.50 (m, 1H), 7.63-7.65 (m, 1H), 7.68-7.70 (m, 1H), 7.76 (s, 1H), 8.21 (s, 1H), 8.36 (s, 2H).

### Example 38. Compound 40

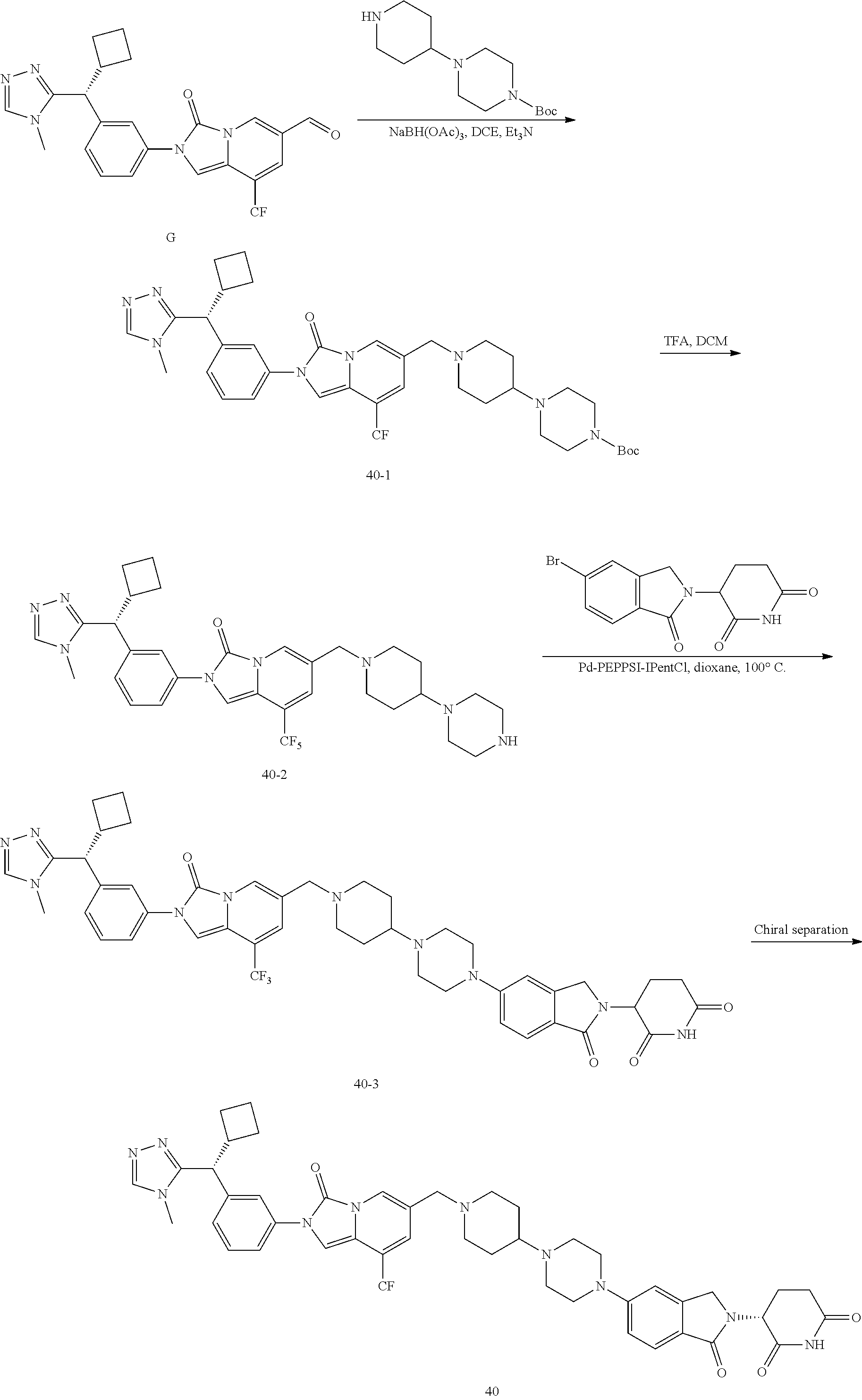

### Synthesis of Compound 40-1.

To a stirred solution of intermediate G (1.5 g, 3.294 mmol, 1 equiv) and tert-butyl 4-(piperidin-4-yl)piperazine-1-carboxylate (1.33 g, 4.941 mmol, 1.5 equiv) in DCE (30 mL) was added NaBH(OAc)₃ (1.40 g, 6.588 mmol, 2 equiv). The resulting mixture was stirred for overnight at room temperature under nitrogen atmosphere. The reaction was quenched by the addition of sat. NaHCO₃ (aq.) (20 mL). The resulting mixture was extracted with CH₂Cl₂ (2×30 mL). The combined organic layers were concentrated under reduced pressure. The residue was purified by reverse flash chromatography with the following conditions: column, C18 silica gel; mobile phase, MeCN in Water (0.10 mmol/L NH₄HCO₃), 25% to 90% gradient in 30 min; detector, UV 254 nm. This resulted in Compound 40-1 (1.4 g, 59.97%) as a yellow solid.

### Synthesis of Compound 40-2.

To a stirred solution of Compound 40-1 (1.4 g, 1.975 mmol, 1 equiv) in DCM (10 mL) was added TFA (3 mL). The resulting mixture was stirred for 3 h at room temperature. The resulting mixture was concentrated under reduced pressure. The residue was purified by Prep-TLC (CH₂Cl₂/MeOH 10:1) to afford Compound 40-2 (1 g, 83.18%) as a yellow solid.

### Synthesis of Compound 40-3.

To a stirred solution of Compound 40-2 (1.06 g, 3.286 mmol, 2 equiv) in dioxane (30 mL) were added Cs₂CO₃ (1.61 g, 4.929 mmol, 3 equiv) and Pd PEPPSI IPentCl (0.14 g, 0.164 mmol, 0.1 equiv) under nitrogen atmosphere. The resulting mixture was stirred for overnight at 100° C. under nitrogen atmosphere. The reaction was quenched by the addition of Water/HOAc=10/1 (50 mL). The aqueous layer was extracted with EtOAc (3×50 mL). The aqueous layer was concentrated under reduced pressure. The residue was purified by reverse flash chromatography with the following conditions: column, C18 silica gel; mobile phase, MeCN in Water (0.1% TFA), 5% to 45% gradient in 30 min; detector, UV 220 nm. The crude product (600 mg) was purified by Prep-HPLC with the following conditions (Column: Xselect CSH C18 OBD Column 30*150 mm 5 μm, n; Mobile Phase A: Water (0.05% TFA), Mobile Phase B: ACN; Flow rate: 60 mL/min; Gradient: 12% B to 23% B in 8 min, 23% B; Wave Length: 254; 220 nm; RT1 (min): 7.61) to afford Compound 40-3 (210 mg, 15.02%) as a yellow solid.

### Synthesis of Compound 40.

Compound 40-3 (210 mg) was separated by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 60% B to 60% B in 16 min; Wave Length: 220/254 nm; RT1 (min): 7.96; RT2 (min): 12.76; first peak was product) to afford Compound 40 (63.1 mg, 30.05%) as a yellow solid.

LCMS-Compound 40: (ES, m/z): [M+H]⁺ 851;

NMR-Compound 40: (400 MHz, DMSO δ ppm): δ1.43-1.46 (m, 2H), 1.68-1.80 (m, 7H), 1.96-1.98 (m, 3H), 2.08-2.10 (m, 2H), 2.21-2.28 (m, 1H), 2.34-2.35 (m, 2H), 2.56-2.65 (m, 5H), 2.87-2.90 (m, 3H), 3.28-3.31 (m, 6H), 3.43 (s, 3H), 4.23-4.35 (m, 3H), 5.03-5.07 (m, 1H), 7.02-7.07 (m, 3H), 7.19-7.21 (d, 1H), 7.33 (s, 1H), 7.43-7.46 (t, 1H), 7.51-7.53 (d, 1H), 7.69-7.74 (m, 3H), 8.34 (s, 1H), 10.97 (s, 1H).

### Example 39. Compound 41

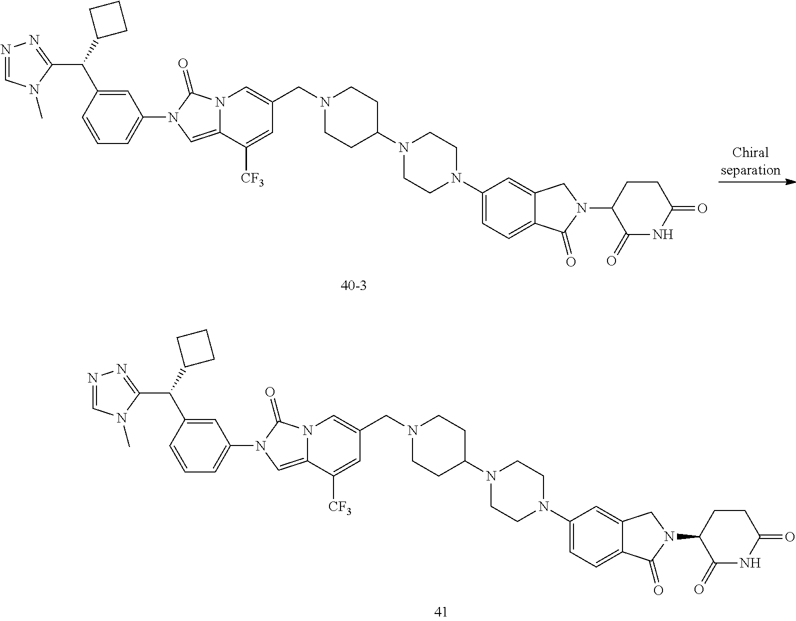 [SMILES: Cn1cnnc1[C@@H](c1cccc(-n2cc3c(C(F)(F)F)cc(CN4CCC(N5CCN(c6ccc7c(c6)CN(C6CCC(=O)NC6=O)C7=O)CC5)CC4)cn3c2=O)c1)C1CCC1 ; Cn1cnnc1[C@@H](c1cccc(-n2cc3c(C(F)(F)F)cc(CN4CCC(N5CCN(c6ccc7c(c6)CN([C@H]6CCC(=O)NC6=O)C7=O)CC5)CC4)cn3c2=O)c1)C1CCC1]

### Synthesis of Compound 41.

Compound 40-3 (210 mg) was separated by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 60% B to 60% B in 16 min; Wave Length: 220/254 nm; RT1 (min): 7.96; RT2 (min): 12.76; second peak was product) to afford (Compound 41 (58.9 mg, 28.05%) as a yellow solid.

LCMS-Compound 41: (ES, m/z): [M+H]⁺ 851:

NMR-Compound 41: (400 MHz, DMSO, δ ppm): δ1.43-1.46 (m, 2H), 1.77-1.81 (m, 7H), 1.95-2.09 (m, 4H), 2.21-2.35 (m, 3H), 2.56-2.68 (m, 5H), 2.87-2.93 (m, 3H), 3.28-3.31 (m, 6H), 3.43 (s, 3H), 4.23-4.35 (m, 3H), 5.03-5.07 (m, 1H), 7.02-7.07 (m, 3H), 7.19-7.21 (d, 1H), 7.32 (s, 1H), 7.43-7.46 (t, 1H), 7.51-7.53 (d, 1H), 7.68-7.74 (m, 3H), 8.33 (s, 1H), 10.97 (s, 1H).

### Example 40. Compound 42

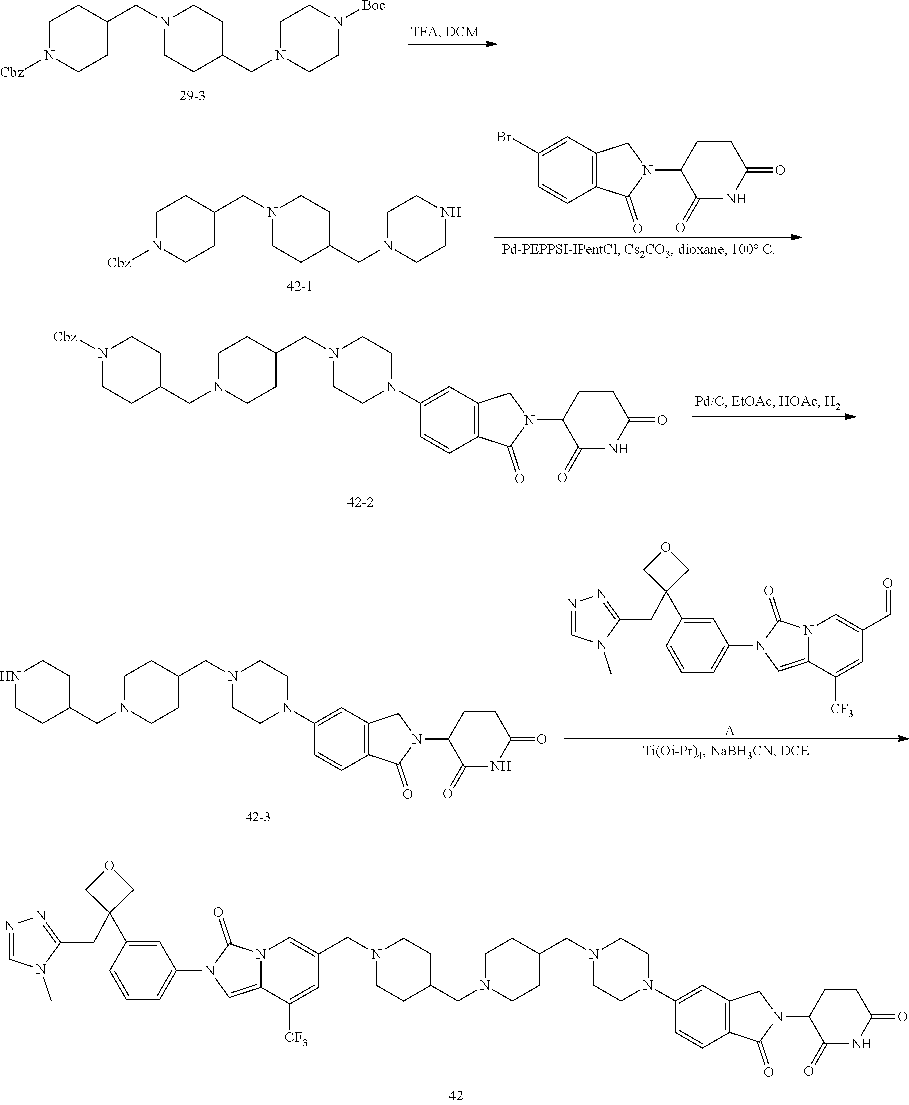

### Synthesis of Compound 42-1.

A solution of Compound 29-3 (4.5 g, 8.74 mmol, 1.0 equiv) and TFA (23 mL) in DCM (4.5 mL) was stirred for overnight at room temperature. The resulting mixture was concentrated under reduced pressure. The residue was basified to pH 8 with NH₃H₂O. The residue was purified by reverse flash chromatography with the following conditions: column, C18 silica gel; mobile phase, MeCN in Water (10 mmol/L NH₄HCO₃), 10% to 90% gradient in 50 min; detector, UV 254 nm. This resulted in Compound 42-1 (1.7 g, 44%) as a brown oil.

### Synthesis of Compound 42-2.

To a stirred solution of Compound 42-1 (1.7 g, 4.24 mmol, 1.0 equiv) and 3-(5-bromo-1-oxo-3H-isoindol-2-yl) piperidine-2,6-dione (2.7 g, 8.49 mmol, 2.0 equiv) in 1,4-dioxane (20 mL) were added Cs₂CO₃ (4.1 g, 12.73 mmol, 3.0 equiv) and Pd-PEPPSI-IPentCl 2-methylpyridine (o-picoline (0.4 g, 0.45 mmol, 0.1 equiv) at room temperature under nitrogen atmosphere. The resulting mixture was stirred for overnight at 100° C. under nitrogen atmosphere. The reaction was quenched by the addition of 10% AcOH (aq.) (100 mL) at room temperature. The resulting mixture was concentrated under reduced pressure. The residue was purified by silica gel column chromatography, eluted with CH₂Cl₂/MeOH (10:1) to afford Compound 42-2 (1.2 g, 43.04%) as a grey solid.

### Synthesis of Compound 42-3.

To a solution of Compound 42-2 (1.2 g, 1.82 mmol, 1.0 equiv) in 30 mL EtOAc and 6 mL AcOH was added Pd/C (10%, 250 mg) under nitrogen atmosphere in a 100 mL round-bottom flask. The mixture was hydrogenated at room temperature for 3 h under hydrogen atmosphere using a hydrogen balloon, filtered through a Celite pad and concentrated under reduced pressure. This resulted in Compound 42-3 (900 mg, 94%) as a grey solid.

### Synthesis of Compound 42.

To a stirred solution of Compound 42-3 (400 mg, 0.87 mmol, 1.0 equiv) and Intermediate A (457 mg, 0.87 mmol, 1.0 equiv) in THF (4 mL) was added Ti(Oi-Pr)₄ (994 mg, 3.49 mmol, 4.0 equiv) at room temperature. The resulting mixture was stirred for overnight at mom temperature. To the above mixture was added NaBH₃CN (164.86 mg, 2.622 mmol, 3 equiv). The resulting mixture was stirred for additional 2 h at room temperature. The reaction was quenched by the addition of 10% AcOH (aq.) (50 mL) at room temperature. The resulting mixture was extracted with CH₂Cl₂ (5×40 mL). The resulting mixture was concentrated under reduced pressure. The crude product (300 mg) was purified by Prep-HPLC with the following conditions (Column: XSelect CSH Prep C18 OBD Column, 19*250 mm, 5 μm; Flow rate: 25 mL/min; Gradient: 2% B to 14% B in 10 min, 14% B; Wave Length: 254/220 nm; RT1 (min): 9.12; Number of Runs: 0) to afford Compound 42 (20 mg, 2%) as a yellow solid.

LC-MS-Compound 42: (ES, m/z): [(M−HCOOH)/2+H]⁺ 482

H-NMR-Compound 42: (400 MHz, CD₃OD, δ ppm): 1.42-1.54 (m, 4H), 1.85-1.96 (m, 4H), 2.07-2.26 (m, 5H), 2.382.39 (m, 2H), 2.51-2.54 (m, 1H), 2.67 (s, 4H), 2.80-2.81 (m, 1H), 2.88-2.92 (m, 1H), 2.95-3.03 (m, 9H), 3.39 (s, 4H), 3.46-3.47 (m, 2H), 3.56-3.58 (m, 2H), 3.68 (s, 2H), 5.07-5.14 (m, 5H), 6.94-6.96 (d, 1H), 7.06-7.09 (m, 1H), 7.10 (s, 1H), 7.28 (s, 1H), 7.46-7.50 (m, 1H), 7.62-7.64 (d, 2H), 7.66 (s, 1H), 8.20 (s, 1H), 8.35 (s, 1H).

### Example 41. Compound 43

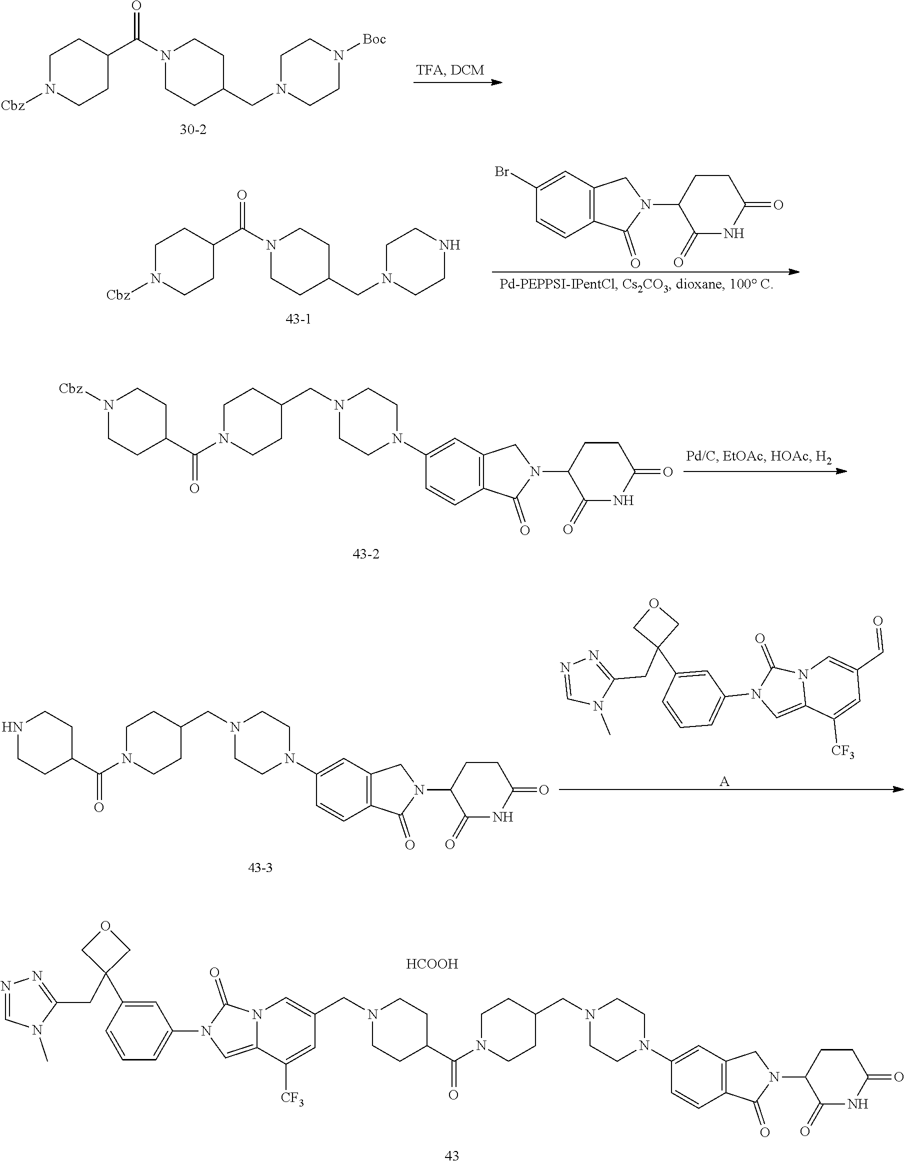

### Synthesis of Compound 43-1.

A solution of Compound 30-2 (2.7 g, 1 equiv) in DCM (30 mL) was treated with TFA (6 mL) for overnight at room temperature under nitrogen atmosphere. The mixture was basified to pH 8 with NH₃·H₂O. The crude product was purified by reverse phase flash with the following conditions (column, C18 silica gel; mobile phase, MeCN in water (10 mmol NH₄HCO₃), 15% to 85% gradient in 40 min; UV 220 nm) to afford Compound 43-1 (2 g, 91%) as a colorless oil.

### Synthesis of Compound 43-2.

To a stirred solution of Compound 43-1 (1.3 g, 3.03 mmol, 1.0 equiv) and 3-(5-bromo-1-oxo-3H-isoindol-2-yl) piperidine-2,6-dione (0.2 g, 6.06 mmol, 2.0 equiv) in 1,4-dioxane (13 mL) were added Cs₂CO₃ (3 g, 9.09 mmol, 3.0 equiv) and Pd PEPPSI IPentCl (0.3 g, 0.30 mmol, 0.1 equiv) at room temperature under nitrogen atmosphere. The resulting mixture was stirred for overnight at 100° C. under nitrogen atmosphere. The reaction was quenched by the addition of 10% AcOH (aq.) (100 mL) at room temperature. The resulting mixture was concentrated under reduced pressure. The residue was purified by silica gel column chromatography, eluted with CH₂Cl₂/MeOH (10:1) to afford Compound 43-2 (800 mg, 39%) as an off-white solid.

### Synthesis of Compound 43-3.

To a solution of Compound 43-2 (700 mg, 1.04 mmol, 1.0 equiv) in 30 mL EtOAc and 6 mL AcOH was added Pd/C (10%, 140 mg) under nitrogen atmosphere in a 100 mL round-bottom flask. The mixture was hydrogenated at room temperature for 3 h under hydrogen atmosphere using a hydrogen balloon, filtered through a Celite pad and concentrated under reduced pressure. This resulted in Compound 43-3 (550 mg, 98%) as a grey oil.

### Synthesis of Compound 43.

To a stirred solution of Compound 43-3 (400 mg, 0.87 mmol, 1.0 equiv) and Intermediate A (703 mg, 1.31 mmol, 1.5 equiv) in THF (4 mL) were added Ti(Oi-Pr)₄ (994 mg, 3.49 mmol, 4.0 equiv) and NaBH₃CN (164 mg, 2.62 mmol, 3.0 equiv) at room temperature. The resulting mixture was stirred for overnight at room temperature. The reaction was quenched by the addition of 10% AcOH (aq.) (30 mL) at room temperature. The aqueous layer was extracted with CH₂Cl₂ (5×30 mL). The resulting mixture was concentrated under reduced pressure. The crude product (200 mg) was purified by Prep-HPLC with the following conditions (Column: XBridge Shield RP18 OBD Column, 30*150 mm, 5 μm; Mobile Phase A: water (0.1% FA), Mobile Phase B: ACN; Flow rate: 60 mL/min; Gradient: 20% B to 45% B in 8 min, 45% B; Wave Length: 220 nm; RT1 (min): 7.78; Number of Runs: 0) to afford Compound 43 (15 mg, 2%) as a yellow solid.

LC-MS-Compound 43: (ES, m/z): [(M−HCOOH)/2+H]⁺ 489

H-NMR-Compound 43: (400 MHz, CD₃OD, δ ppm): 1.02-1.20 (m, 2H), 1.83-1.89 (m, 5H), 1.94-1.97 (m, 2H), 2.15-2.18 (m, 1H), 2.42-2.50 (m, 5H), 2.67-2.71 (m, 1H), 2.75-2.88 (m, 6H), 2.90-2.93 (m, 1H), 2.96 (s, 3H), 3.15-3.18 (m, 3H), 3.42 (s, 4H), 3.56-3.58 (m, 2H), 3.68 (s, 2H), 4.06-4.10 (m, 1H), 4.40-4.43 (m, 1H), 4.54-4.57 (m, 1H), 5.08-5.14 (m, 5H), 6.91-6.93 (d, 1H), 7.10-7.15 (m, 4H), 7.31 (s, 1H), 7.46-7.50 (t, 1H), 7.62-7.67 (t, 2H), 7.76 (s, 1H), 8.20 (s, 1H), 8.35 (s, 2H).

### Example 42. Compound 44

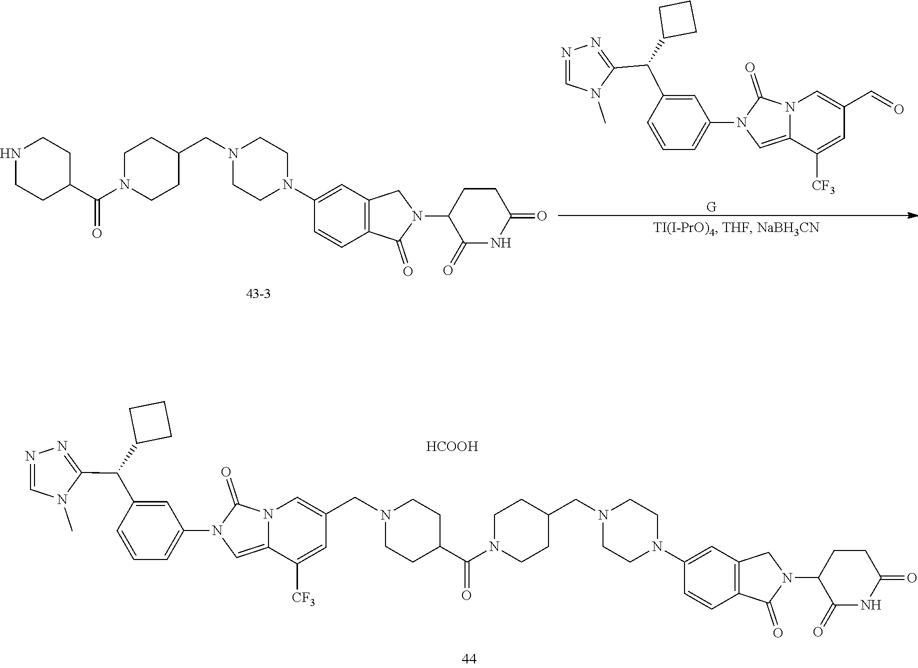

### Synthesis of Compound 44.

To a stirred solution of Compound 43-3 (650 mg, 1.42 mmol, 1.0 equiv) and Intermediate G (1148 mg, 2.14 mmol, 1.5 equiv) in THF (7 mL) was added Ti(Oi-Pr)₄ (1216.9 mg, 4.21 mmol, 3.0 equiv) at room temperature. The resulting mixture was stirred for overnight at room temperature. To the above mixture was added NaBH₃CN (179 mg, 2.85 mmol, 2.0 equiv) at room temperature. The resulting mixture was stirred for additional 2 h at room temperature. The reaction was quenched by the addition of 10% AcOH (aq.) (100 mL) at room temperature. The resulting mixture was concentrated under reduced pressure. The crude product (300 mg) was purified by Prep-HPLC with the following conditions (Column: Xselect CSH C18 OBD Column 30*150 mm 5 μm, n; Mobile Phase A: water (0.1% FA), Mobile Phase B: ACN; Flow rate: 60 mL/min; Gradient: 8% B to 18% B in 8 min, 18% B; Wave Length: 254; 220 nm; RT1 (min): 7.63; Number of Runs: 0) to afford Compound 44 (21 mg, 2%) as a yellow solid.

LC-MS-Compound 44: (ES, m/z): [(M−HCOOH)/2+H]⁺ 488

1H-NMR-Compound 44: (400 MHz, CD₃OD, δ ppm): 1.05-1.24 (m, 2H), 1.319 (s, 1H), 1.77-1.96 (m, 12H), 2.18-2.20 (m, 1H), 2.21-2.26 (m, 1H), 2.37-2.38 (m, 4H), 2.46-2.50 (m, 1H), 2.70-2.79 (m, 5H), 2.80-2.81 (m, 2H), 2.88-2.91 (m, 1H), 3.12-3.14 (m, 3H), 3.33 (s, 4H), 3.40 (s, 2H), 3.52-3.55 (m, 3H), 4.28-4.31 (m, 1H), 4.41-4.42 (m, 1H), 4.45-4.50 (m, 2H), 4.55-4.56 (m, 1H), 5.10-5.14 (m, 1H), 7.09-7.14 (m, 4H), 7.27-7.29 (d, 1H), 7.48-7.52 (t, 1H), 7.64-7.66 (m, 3H), 7.73 (s, 1H), 8.41 (s, 1H).

### Example 43. Compound 45

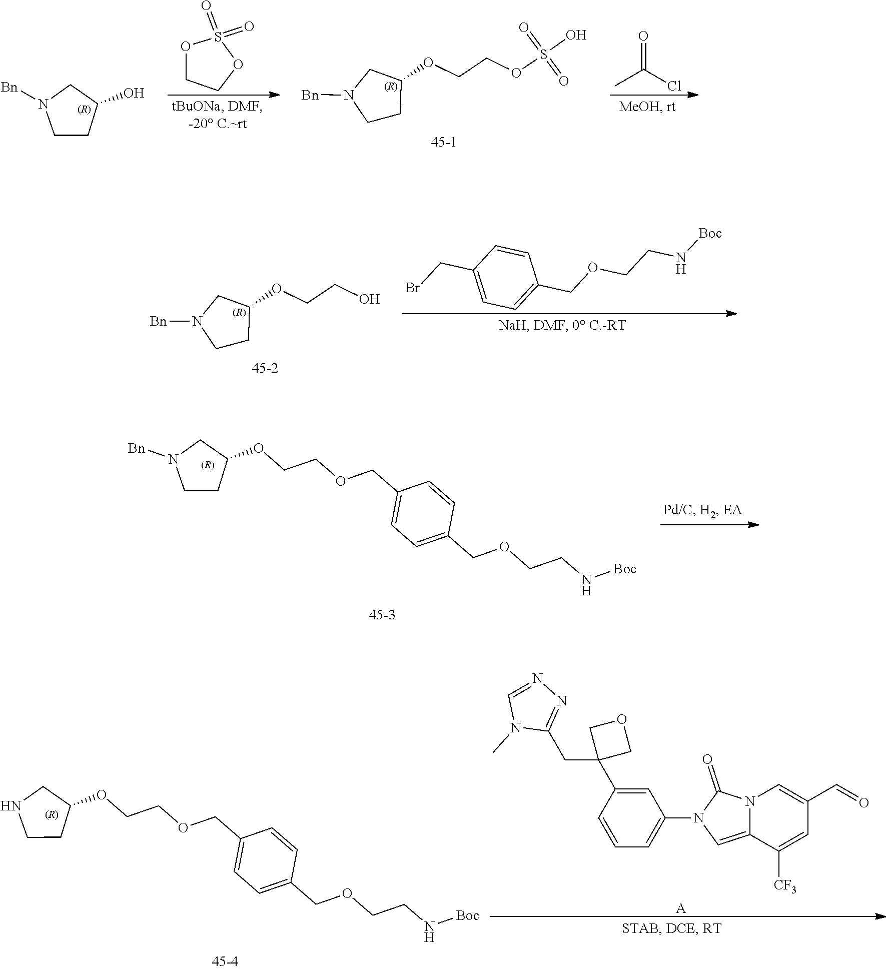

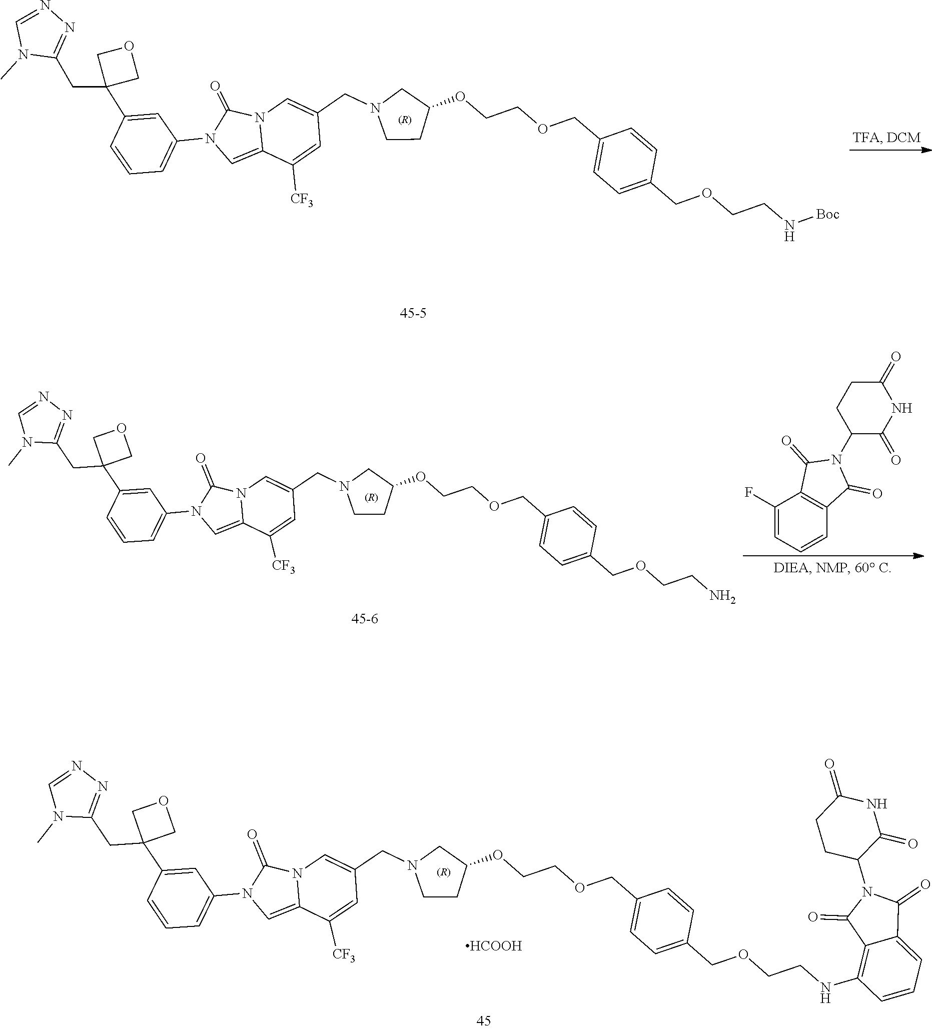

### Synthesis of Compound 45-1.

Into a 100 mL 3-necked round-bottom flask were added (3R)-1-benzylpyrrolidin-3-ol (10 g, 56.418 mmol, 1 equiv), DMF (15 mL), 1,3,2lambda6-dioxathiolane-2,2-dione (17.51 g, 141.045 mmol, 2.5 equiv) and t-BuONa (13.56 g, 141.045 mmol, 2.5 equiv) at −20° C. The resulting mixture was stirred for 4 h at room temperature under nitrogen atmosphere. The reaction was quenched by the addition of water/ice (100 mL) at room temperature. The aqueous layer was extracted with EtOAc (2×50 mL). The aqueous phase was concentrated under reduced pressure. The Compound 45-1 (15 g, 88.22%) as an off-white solid was used in the next step directly without further purification.

### Synthesis of Compound 45-2.

Into a 500 mL 3-necked round-bottom flask were added Compound 45-1 (15 g, 49.774 mmol, 1 equiv), MeOH (75 mL) and acetyl chloride (75 mL) at room temperature. The resulting mixture was stirred for 2 h at room temperature. The reaction was quenched by the addition of sat. NaHCO₃ (aq.) (100 mL) at room temperature. The aqueous layer was extracted with EtOAc (3×50 mL). The resulting mixture was concentrated under reduced pressure. The residue was purified by silica gel column chromatography, eluted with PE/EA (5:1) to afford Compound 45-2 (3 g, 27.24%) as an off-white solid.

### Synthesis of Compound 45-3.

Into a 250 mL 3-necked round-bottom flask were added Compound 45-2 (3 g, 13.556 mmol, 1 equiv). DMF (30 mL) and NaH (0.39 g, 16.267 mmol, 1.2 equiv) at 0° C. The resulting mixture was stirred for 1 h at 0° C. To the above mixture was added tert-butyl N-(2-{[4-(bromomethyl)phenyl]methoxy}ethyl)carbamate (4.67 g, 13.556 mmol, 1 equiv) at 0° C. The resulting mixture was stirred for additional overnight at room temperature. The reaction was quenched by the addition of sat. NH₄Cl (aq.) (100 mL) at room temperature. The aqueous layer was extracted with EtOAc (3×60 mL). The resulting mixture was concentrated under reduced pressure. The residue was purified by silica gel column chromatography, eluted with PE/EA (1:1) to afford Compound 45-3 (2 g, 30.44%) as an off-white solid.

### Synthesis of Compound 45-4.

To a solution of Compound 45-3 (2 g, 4.127 mmol, 1 equiv) in 25 mL EA was added Pd/C (10%, 0.44 g) under nitrogen atmosphere in a 100 mL round-bottom flask. The mixture was hydrogenated at room temperature for overnight under hydrogen atmosphere using a hydrogen balloon, filtered through a Celite pad and concentrated under reduced pressure to afford Compound 45-4 (1.1 g, 67.56%) as an off-white solid.

### Synthesis of Compound 45-5.

Into a 100 mL round-bottom flask were added Compound 45-4 (1.1 g, 2.788 mmol, 1 equiv), DCE (20 mL), Intermediate A (1.28 g, 2.788 mmol, 1 equiv), STAB (1.18 g, 5.576 mmol, 2 equiv) and AcOH (0.17 g, 2.788 mmol, 1 equiv) at room temperature. The resulting mixture was stirred for 2 h at room temperature. The reaction was quenched by the addition of sat. NH₄Cl (aq.) (100 mL) at room temperature. The aqueous layer was extracted with CH₂Cl₂ (3×60 mL). The resulting mixture was concentrated under reduced pressure. The residue was purified by silica gel column chromatography, eluted with CH₂Cl₂/MeOH (10:1) to afford Compound 45-5 (1 g, 42.90%) as a light yellow solid.

### Synthesis of Compound 45-6.

Into a 100 mL round-bottom flask were added Compound 45-5 (1 g, 1.196 mmol, 1 equiv), DCM (9 mL) and TFA (3 mL) at room temperature. The resulting mixture was stirred for 2 h at room temperature. The resulting mixture was concentrated under reduced pressure. The residue was purified by Prep-TLC (CH₂Cl₂/MeOH 10:1) to afford Compound 45-6 (500 mg, 56.80%) as a light yellow solid.

### Synthesis of Compound 45.

Into a 100 mL round-bottom flask were added Compound 45-6 (420 mg, 0.571 mmol, 1 equiv), NMP (5 mL), 2-(2,6-dioxopiperidin-3-yl)-4-fluoroisoindole-1,3-dione (315.34 mg, 1.142 mmol, 2 equiv) and DIEA (147.55 mg, 1.142 mmol, 2 equiv) at room temperature. The resulting mixture was stirred for overnight at 70° C. The crude product (5 mL) was purified by Prep-HPLC with the following conditions (Column: Xcelect CSH F-pheny OBD Column, 19*250 mm, 5 μm; Mobile Phase A: Water (0.1% FA), Mobile Phase B: ACN; Flow rate: 25 mL/min; Gradient: 28% B to 32% B in 10 min; Wave Length: 254/220 nm; RT1 (min): 10.5) to afford Compound 45-7 (5.3 mg, 0.94%) as a light yellow solid.

LC-MS-Compound 45: (ES, m/z): [M−HCOOH+H]⁺ 992

H-NMR-Compound 45: 1H NMR (300 MHz, CD₃OD-d4 ppm) δ 1.82-1.98 (s, 1H), 2.03-2.22 (d, 2H), 2.61-2.93 (m, 7H), 2.92-3.01 (m, 3H), 3.49-3.52 (m, 2H), 3.56-3.65 (m, 6H), 3.62-3.72 (m, 4H), 4.02-4.21 (s, 1H), 4.56-4.62 (m, 4H), 5.01-5.14 (m, 5H), 6.90-7.10 (m, 3H), 7.10-7.14 (d, 2H), 7.25-7.39 (m, 5H), 7.42-7.51 (t, 2H), 7.63-7.82 (m, 2H).

### Example 44. Compound 46

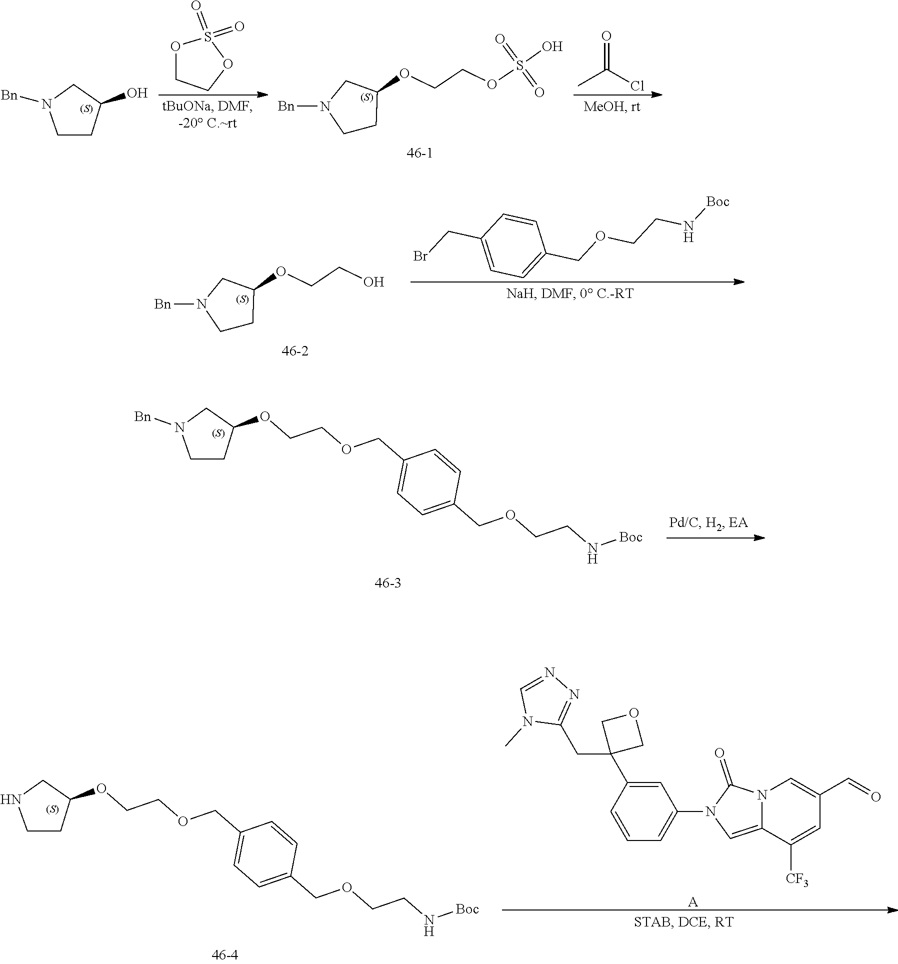

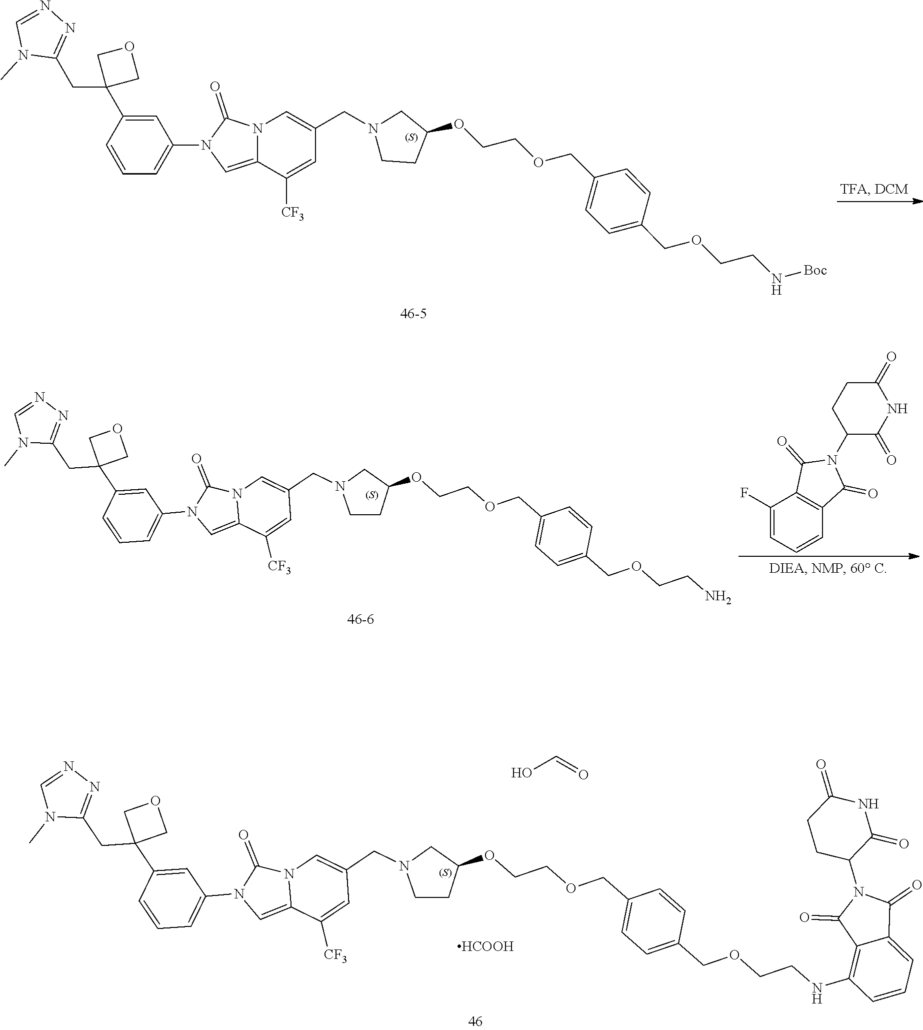

### Synthesis of Compound 46-1.

A solution of (3S)-1-benzylpyrrolidin-3-ol (10 g, 56.418 mmol, 1 equiv) in DMF (100 mL) was treated with 1,3,2-lambda6-dioxathiolane-2,2-dione (17.51 g, 141.045 mmol, 2.5 equiv) at −20° C. under nitrogen atmosphere followed by the addition of t-BuONa (13.56 g, 141.045 mmol, 2.5 equiv) at −20° C. The resulting mixture was stirred for 4 h at room temperature. The reaction was quenched by the addition of water (200 mL) at room temperature. The resulting mixture was washed with 3×100 mL of DCM. The resulting mixture was concentrated under reduced pressure. The crude product was used in the next step directly without further purification. This resulted in Compound 46-1 (15 g, 61.76%) as a Brown yellow oil.

### Synthesis of Compound 46-2.

To a stirred solution of acetyl chloride (20 mL) in MeOH (140 mL) was added Compound 46-1 (15 g, 49.774 mmol, 1 equiv) at room temperature. The resulting mixture was stirred for 3 h at room temperature. The mixture was acidified to pH 7 with saturated NaHCO₃ (aq.) 200 mL. The aqueous layer was extracted with MTBE (3×250 mL). The resulting mixture was concentrated under reduced pressure. The residue was purified by silica gel column chromatography, eluted with PE/EA (1:1) to afford Compound 46-2 (5.3 g, 43.30%) as a yellow oil.

### Synthesis of Compound 46-3.

A solution of Compound 46-2 (5.3 g, 23.949 mmol, 1 equiv) in THF (60 mL) was treated with NaH (0.86 g, 35.924 mmol, 1.5 equiv) for 1 h at 0° C. To the above mixture was added tert-butyl N-(2-{[4-(bromomethyl) phenyl] methoxy} ethyl) carbamate (9.89 g, 28.739 mmol, 1.2 equiv) at room temperature. The resulting mixture was stirred for overnight at room temperature. The reaction was quenched by the addition of sat. NH₄Cl (aq.) (100 mL) at room temperature. The aqueous layer was extracted with EtOAc (3×50 mL). The resulting mixture was concentrated under reduced pressure. The residue was purified by silica gel column chromatography, eluted with PE/EA (5:1) to afford Compound 46-3 (4 g, 31.71%) as a yellow oil.

### Synthesis of Compound 46-4.

To a solution of Compound 46-3 (2 g, 4.127 mmol, 1 equiv) in 40 mL MeOH was added Pd/C (10%, 0.2 g) under nitrogen atmosphere in a 250 mL round-bottom flask. The mixture was hydrogenated at room temperature for 4 h under hydrogen atmosphere using a hydrogen balloon, filtered through a Celite pad and concentrated under reduced pressure. This resulted in Compound 46-4 (1.3 g, 59.89%) as a light yellow oil.

### Synthesis of Compound 46-5.

A solution of Compound 46-4 (1.2 g, 3.042 mmol, 1 equiv) and Intermediate A (1.39 g, 3.042 mmol, 1 equiv) in DCE (15 mL) was stirred for 1 h at room temperature. To the above mixture was added STAB (1.29 g, 6.084 mmol, 2 equiv) at room temperature. The resulting mixture was stirred for overnight at room temperature. The residue was purified by Prep-TLC (CH₂Cl₂/MeOH 10:1) to afford Compound 46-5 (800 mg, 29.89%) as a yellow oil.

### Synthesis of Compound 46-6.

To a stirred mixture of TFA (3 mL) in DCM (9 mL) was added Compound 46-5 (800 mg, 0.957 mmol, 1 equiv) at room temperature. The resulting mixture was stirred for 1 h at room temperature. The resulting mixture was concentrated under reduced pressure. The residue was purified by Prep-TLC (CH₂Cl₂/MeOH 10:1) to afford Compound 46-6 (500 mg, 68.16%) as a yellow solid.

### Synthesis of Compound 46.

Into a 100 mL round-bottom flask were added Compound 46-6 and 2-(2,6-dioxopiperidin-3-yl)-4-fluoroisoindole-1,3-dione (540.58 mg, 1.956 mmol, 3 equiv) in NMP (5 mL) at room temperature. To the above mixture was added DIEA (126.47 mg, 0.978 mmol, 1.5 equiv) at room temperature. The resulting mixture was stirred for overnight at 60° C. The crude product was purified by Prep-HPLC with the following conditions (Column: Xcelect CSH F-pheny OBD Column, 19*250 mm, 5 μm; Mobile Phase A: Water (0.1% FA), Mobile Phase B: ACN; Flow rate: 25 mL/min; Gradient: 18% B to 32% B in 12 min, 32% B; Wave Length: 254/220 nm; RT1 (min): 11) to afford Compound 46 (6.4 mg, 0.95%) as a yellow solid.

LC-MS-Compound 46: (ES, m/z): [M−HCOOH+H]⁺ 992

H-NMR-Compound 46: (400 MHz, CD3OD-d6, ppm): δ1.82-1.98 (s, 1H), δ2.03-2.22 (d, 2H), δ2.61-2.70 (s, 1H), δ2.71-2.93 (m, 6H), δ2.92-3.01 (m, 3H), δ3.49-3.55 (m, 3H), δ3.56-3.60 (s, 1H), δ3.61-3.65 (m, 4H), δ3.62-3.72 (m, 4H), δ4.02-4.21 (s, 1H), δ4.56-4.62 (m, 4H), δ5.01-5.14 (m, 5H), δ6.90-7.10 (m, 3H), δ7.10-7.14 (d, 2H), δ7.25-7.39 (m, 5H), δ7.42-7.51 (t, 3H), δ7.63-7.71 (s, 1H), δ8.43 (s, 1H).

### Example 45. Compound 47

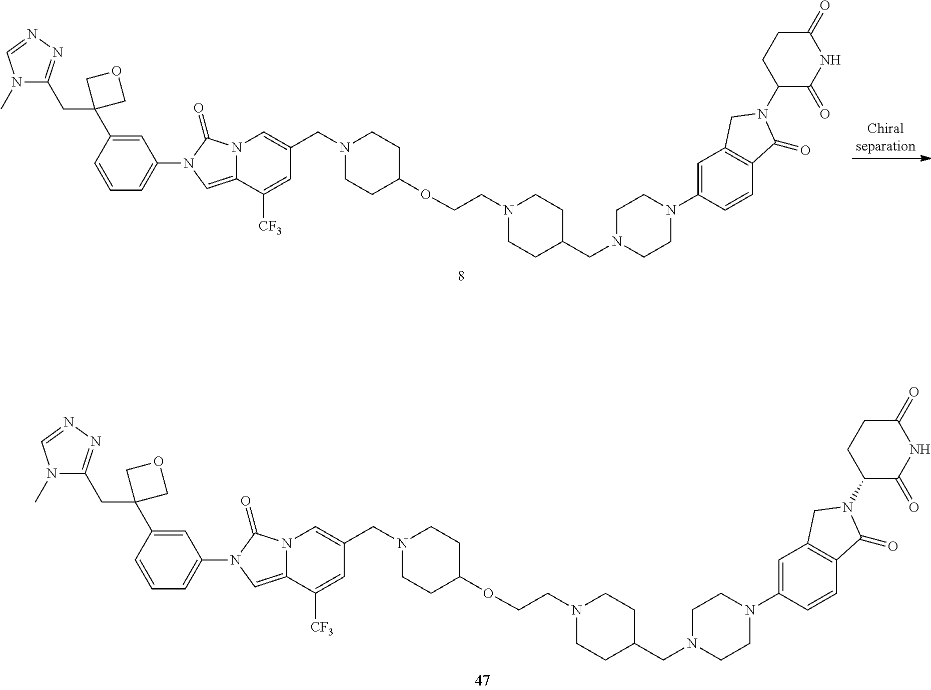 [SMILES: Cn1cnnc1CC1(c2cccc(-n3cc4c(C(F)(F)F)cc(CN5CCC(OCCN6CCC(CN7CCN(c8ccc9c(c8)CN(C8CCC(=O)NC8=O)C9=O)CC7)CC6)CC5)cn4c3=O)c2)COC1 ; Cn1cnnc1CC1(c2cccc(-n3cc4c(C(F)(F)F)cc(CN5CCC(OCCN6CCC(CN7CCN(c8ccc9c(c8)CN([C@@H]8CCC(=O)NC8=O)C9=O)CC7)CC6)CC5)cn4c3=O)c2)COC1]

### Synthesis of Compound 47.

Compound 8 (300 mg) was purified by Prep-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 60% B to 60% B in 25 min; Wave Length: 220/254 nm; RT1 (min): 11.09; RT2 (min): 20.33; the first peak was product) to afford Compound 47 (72.6 mg, 25.32%) as a yellow solid.

LCMS-Compound 47: (ES, m/z): [M+H]⁺ 994

NMR-Compound 47: (400 MHz, DMSO, δ ppm): δ1.02-1.11 (m, 2H), 1.42-1.45 (m, 3H), 1.65-1.68 (m, 2H), 1.75-1.85 (m, 2H), 1.95-1.98 (m, 3H), 2.08-2.16 (m, 4H), 2.32-2.40 (m, 1H), 2.46-2.50 (m, 6H), 2.52-2.62 (m, 1H), 2.63-2.67 (m, 2H), 2.85-2.90 (m, 3H), 2.97 (s, 3H), 3.27-3.30 (m, 7H), 3.48-3.53 (m, 4H), 4.18-4.22 (d, 1H), 4.31-4.35 (d, 1H), 4.91-4.96 (m, 4H), 5.06-5.08 (m, 1H), 6.88-6.90 (d, 1H), 7.01-7.06 (m, 3H), 7.32 (s, 1H), 7.38-7.42 (m, 2H), 7.50-7.53 (d, 1H), 7.66 (s, 1H), 7.75-7.70 (d, 1H), 8.20 (s, 1H), 10.95 (s, 1H).

### Example 46. Compound 48

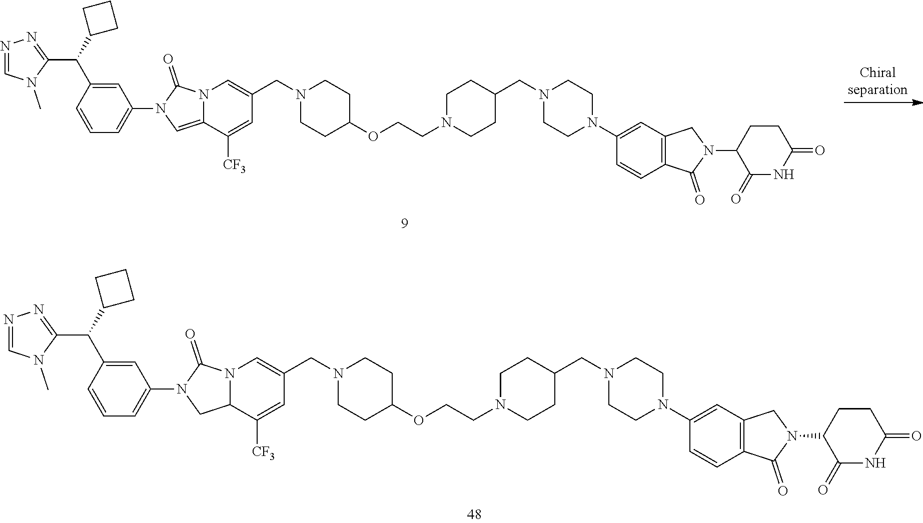

### Synthesis of Compound 48.

Compound 9 (300 mg, 0.302 mmol, 1 equiv)) was separated by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 60% B to 60% B in 12 min; Wave Length: 220/254 nm; RT1 (min): 5.90; RT2 (min): 9.67; the first peak was product) to afford Compound 48 (72.6 mg, 24.20%) as a yellow solid.

LCMS-Compound 48: (ES, m/z): [M+H]⁺ 993

NMR-Compound 48: (400 MHz, DMSO, δ ppm): δ 1.04-1.12 (m, 2H), 1.43-1.52 (m, 3H), 1.65-1.83 (m, 10H), 1.95-1.98 (m, 2H), 2.08-2.16 (m, 5H), 2.32-2.40 (m, 1H), 2.41-2.50 (m, 5H), 2.51-2.67 (m, 4H), 2.82-2.91 (m, 3H), 3.17-3.28 (m, 8H), 3.43 (s, 3H), 3.46-3.52 (m, 2H), 4.18-4.35 (m, 3H), 5.03-5.06 (m, 1H), 7.01-7.07 (m, 3H), 7.19-7.21 (d, 1H), 7.32 (s, 1H), 7.42-7.46 (t, 1H), 7.51-7.53 (m, 1H), 7.66-7.73 (m, 3H), 8.33 (s, 1H), 10.94 (s, 1H).

### Example 47. Compound 49

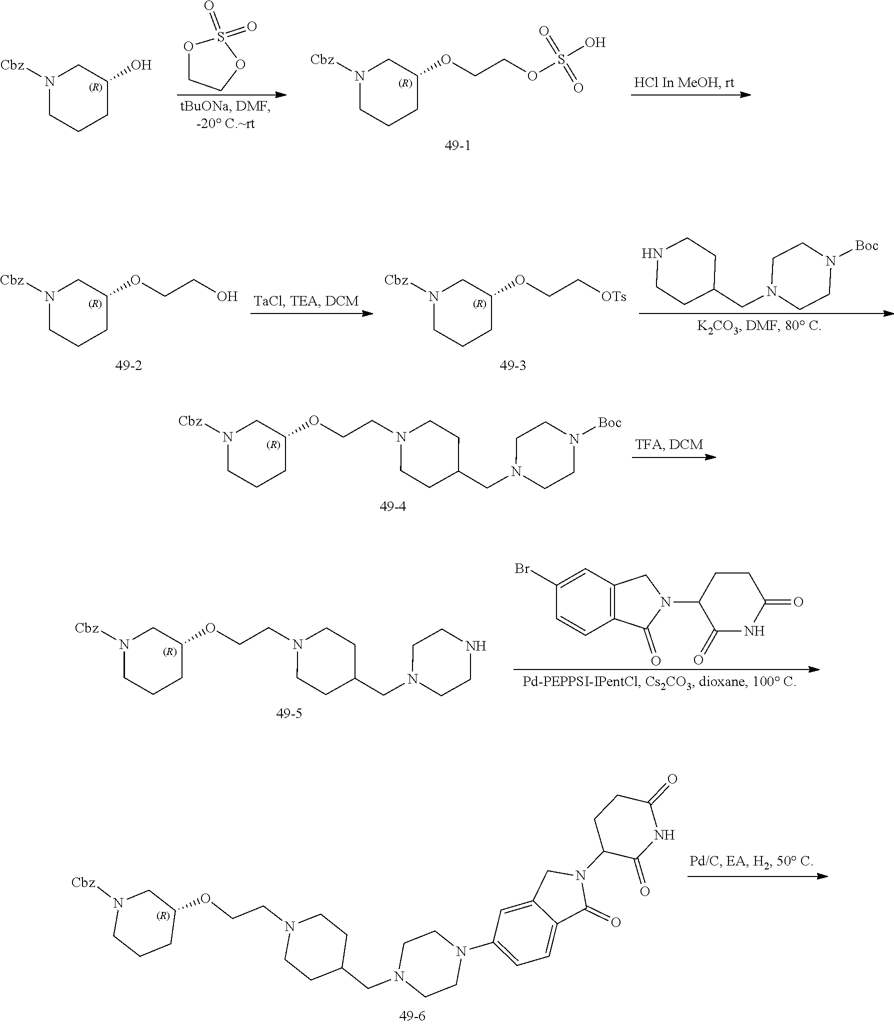

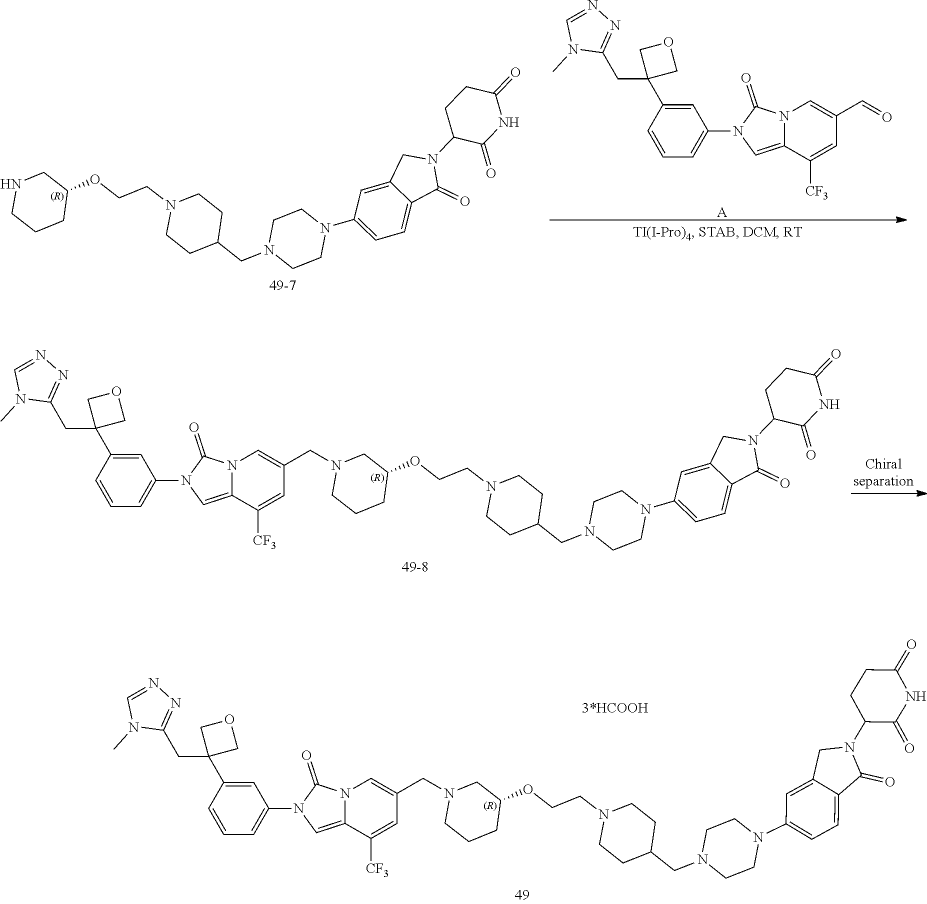

### Synthesis of Compound 49-1.

Into a 500 mL 3-necked round-bottom flask were added benzyl (3R)-3-hydroxypiperidine-1-carboxylate (20 g, 85.004 mmol, 1 equiv), DMF (250 mL), 1,3,2lambda6-dioxathiolane-2,2-dione (15.82 g, 127.506 mmol, 1.5 equiv) and t-BuONa (16.34 g, 170.008 mmol, 2 equiv) at −20° C. The resulting mixture was stirred for 2 h at room temperature. The reaction was quenched by the addition of water (250 mL) at room temperature. The aqueous layer was extracted with EtOAc (3×150 mL). The aqueous phase was concentrated under reduced pressure. The crude product Compound 49-1 (22 g, 72.01%) as an off-white solid was used in the next step directly without further purification.

### Synthesis of Compound 49-2.

Into a 500 mL 3-necked round-bottom flask were added Compound 49-1 (20 g, 55.650 mmol, 1 equiv) and HCl(g) in MeOH (200 mL) at room temperature. The resulting mixture was stirred for overnight at room temperature. The reaction was quenched by the addition of sat. NaHCO₃ (aq.) (150 mL) at room temperature. The aqueous layer was extracted with EtOAc (3×100 mL). The resulting mixture was concentrated under reduced pressure. The residue was purified by silica gel column chromatography, eluted with PE/EA (6:1) to afford Compound 49-2 (13 g, 83.63%) as an off-white solid.

### Synthesis of Compound 49-3.

Into a 500 mL 3-necked round-bottom flask were added Compound 49-2, DCM (150 mL), TEA (14.13 g, 139.617 mmol, 3 equiv) and TsCl (13.31 g, 69.809 mmol, 1.5 equiv) at room temperature. The resulting mixture was stirred for 1 h at room temperature. The reaction was quenched by the addition of sat. NaHCO₃ (aq.) (100 mL) at room temperature. The aqueous layer was extracted with EtOAc (3×80 mL). The resulting mixture was concentrated under reduced pressure. The crude product Compound 49-3 (19 g, 94.17%) off-white solid was used in the next step directly without further purification.

### Synthesis of Compound 49-4.

Into a 500 mL 3-necked round-bottom flask were added Compound 49-3 (19 g, 43.827 mmol, 1 equiv), DMF (200 mL), tert-butyl 4-(piperidin-4-ylmethyl)piperazine-1-carboxylate (12.42 g, 43.827 mmol, 1 equiv) and K₂CO₃ (18.17 g, 131.481 mmol, 3 equiv) at room temperature. The resulting mixture was stirred for overnight at 80° C. The reaction was quenched by the addition of water (500 mL) at room temperature. The aqueous layer was extracted with EtOAc (3×300 mL). The resulting mixture was concentrated under reduced pressure. The residue was purified by silica gel column chromatography, eluted with PE/EA (5:1) to afford Compound 49-4 (12 g, 50.26%) as an off-white solid.

### Synthesis of Compound 49-5.

Into a 500 mL round-bottom flask were added Compound 49-4 (12 g, 22.029 mmol, 1 equiv), DCM (90 mL) and TFA (30 mL) at room temperature. The resulting mixture was stirred for 2 h at room temperature. The resulting mixture was concentrated under reduced pressure. The residue was purified by Prep-TLC (CH₂Cl₂/MeOH 10:1) to afford Compound 49-5 (6 g, 61.26%) as an off-white solid.

### Synthesis of Compound 49-6.

Into a 500 mL 3-necked round-bottom flask were added Compound 49-5 (2 g, 4.498 mmol, 1 equiv), dioxane (20 mL), 3-(5-bromo-1-oxo-3H-isoindol-2-yl) piperidine-2,6-dione (1.45 g, 4.498 mmol, 1 equiv), Cs₂CO₃ (2.93 g, 8.996 mmol, 2 equiv) and Pd-PEPPSI-IPentCl (0.38 g, 0.450 mmol, 0.1 equiv) at room temperature. The resulting mixture was stirred for overnight at 100° C. under nitrogen atmosphere. The reaction was quenched by the addition of 10% AcOH (aq.) (150 mL) at room temperature. The aqueous layer was extracted with CH₂Cl₂/MeOH (10:1) (3×100 mL). The resulting mixture was concentrated under reduced pressure. The residue was purified by Prep-TLC (CH₂Cl₂/MeOH 10:1) to afford Compound 49-6 (1.4 g, 45.31%) as an off-white solid.

### Synthesis of Compound 49-7.

To a solution of Compound 49-6 (1.4 g, 2.038 mmol, 1 equiv) in 20 mL EA was added Pd/C (10%, 0.43 g) under nitrogen atmosphere in a 250 mL round-bottom flask. The mixture was hydrogenated at 50° C. for overnight under hydrogen atmosphere using a hydrogen balloon, filtered through a Celite pad and concentrated under reduced pressure to afford Compound 49-7 (900 mg, 79.89%) as an off-white solid.

### Synthesis of Compound 49-8.

Into a 100 mL round-bottom flask were added Compound 49-7 (880 mg, 1.592 mmol, 1 equiv), DCM (10 mL), Intermediate A (728.26 mg, 1.592 mmol, 1 equiv) and Ti(Oi-Pr)₄ (905.03 mg, 3.184 mmol, 2 equiv) at room temperature. The resulting mixture was stirred for overnight at room temperature. To the above mixture was added STAB (674.87 mg, 3.184 mmol, 2 equiv) at room temperature. The resulting mixture was stirred for additional 2 h at room temperature. The reaction was quenched by the addition of water (20 mL) at room temperature. The aqueous layer was extracted with EtOAc (3×20 mL). The resulting mixture was concentrated under reduced pressure. The residue was purified by Prep-TLC (CH₂Cl₂/MeOH 10:1) to afford Compound 49-8 (300 mg, 18.95%) as an off-white solid.

### Synthesis of Compound 49.

The Compound 49-8 (300 mg, 0.302 mmol, 1 equiv) was purified by Prep-Chiral-HPLC with the following conditions (Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 60% B to 60% B in 25 min; Wave Length: 220/254 nm; RT1 (min): 11.02; RT2 (min): 19.05; The first peak was the product. Sample Solvent: DCM:ACN=1:1—HPLC; Injection Volume: 1 mL; Number of Runs: 5) to afford crude product. The crude product was purified by reversed-phase flash chromatography with the following conditions: column, 40 g C18 silica gel; mobile phase, MeCN in water (0.1% FA), 20% to 55% gradient in 8 min; detector, UV 254 nm to afford Compound 49 (67.9 mg, 19.32%) as a yellow solid.

LC-MS-Compound 49: (ES, m/z): [M−3·HCOOH+H]⁺ 994

H-NMR-Compound 49: 1H NMR (400 MHz, DMSO-d6 ppm) δ 1.15-1.21 (m, 3H), 1.37-1.45 (m, 1H), 1.63-1.70 (m, 1H), 1.92-2.12 (m, 3H), 2.15-2.43 (m, 4H), 2.44-2.55 (m, 2H), 2.56-2.62 (m, 3H), 2.65-2.71 (m, 4H), 2.72-2.74 (m, 2H), 2.75-2.82 (m, 2H), 2.83-2.90 (m, 2H), 2.95-2.99 (m, 4H), 3.00-3.06 (d, 2H), 3.20-3.30 (m, 4H), 3.31-3.34 (m, 1H), 3.35-3.40 (m, 2H), 3.55-3.60 (m, 2H), 4.10-4.22 (d, 1H), 4.30-4.35 (d, 1H), 4.88-4.98 (m, 4H), 5.00-5.10 (m, 1H), 6.85-6.91 (m, 1H), 6.99-7.08 (m, 3H), 7.30-7.35 (m, 1H), 7.36-7.44 (m, 2H), 7.52-7.54 (d, 1H), 7.69 (s, 1H), 7.72-7.80 (m, 1H), 8.20 (s, 1H) 10.95 (s, 1H).

### Example 48. Compound 50

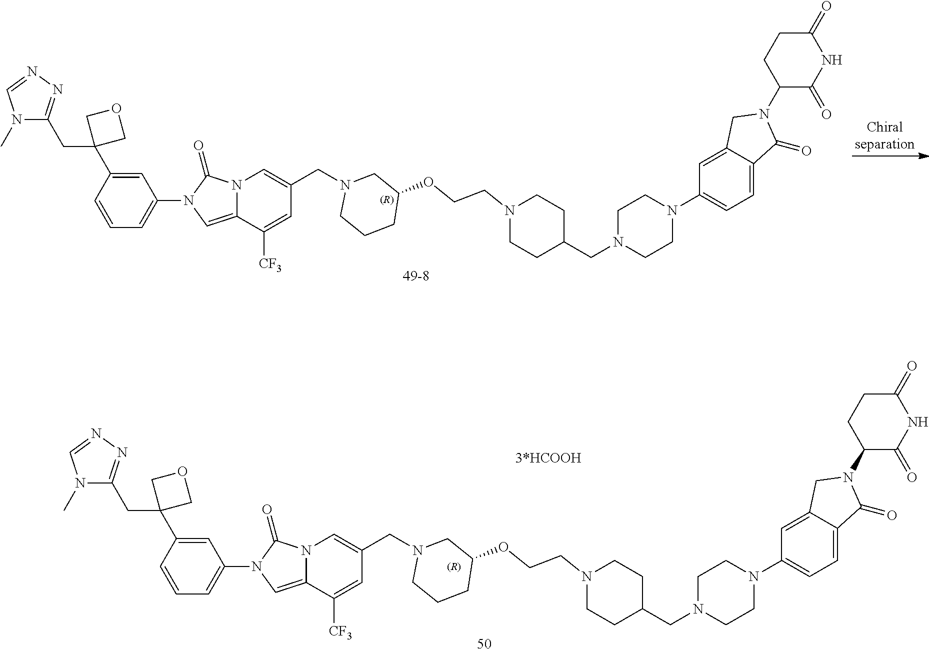

### Synthesis of Compound 50.

The Compound 49-8 (300 mg, 0.302 mmol, 1 equiv) was purified by Prep-Chiral-HPLC with the following conditions (Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 60% B to 60% B in 25 min; Wave Length: 220/254 nm; RT1 (min): 11.02; RT2 (min): 19.05; The second peak was the product. Sample Solvent: DCM:ACN=1:1—HPLC; Injection Volume: 1 mL; Number of Runs: 5 to afford crude product. The crude product was purified by reversed-phase flash chromatography with the following conditions: column, 40 g C18 silica gel; mobile phase, MeCN in water (0.1% FA), 20% to 55% gradient in 8 min; detector, UV 254 nm to afford Compound 50 (64.6 mg, 18.66%) as a yellow solid.

LC-MS-Compound 50: (ES, m/z): [M−3·HCOOH+H]⁺ 994

H-NMR-Compound 50: 1H NMR (400 MHz, DMSO-d6 ppm) δ 1.14-1.20 (m, 3H), 1.37-1.45 (m, 1H), 1.63-1.70 (m, 1H), 1.92-2.12 (m, 3H), 2.15-2.20 (m, 1H), 2.23-2.48 (m, 3H), 2.49-2.52 (m, 2H), 2.55-2.62 (m, 3H), 2.70-2.82 (m, 4H), 2.85-2.90 (m, 2H), 3.00-3.06 (d, 2H), 3.20-3.24 (m, 2H), 3.25-3.30 (m, 3H), 3.31-3.39 (m, 2H), 3.40-3.45 (m, 4H), 3.45-3.47 (m, 2H), 3.49-3.52 (m, 1H), 3.52-3.54 (m, 2H), 3.55-3.60 (m, 2H), 4.10-4.22 (d, 1H), 4.30-4.35 (d, 1H), 4.88-4.98 (m, 4H), 5.00-5.10 (m, 1H), 6.85-6.91 (m, 1H), 6.99-7.08 (m, 3H), 7.30-7.35 (m, 1H), 7.36-7.44 (m, 2H), 7.52-7.54 (d, 1H), 7.69 (s, 1H), 7.72-7.82 (m, 1H), 8.20 (s, 1H), 11.01 (s, 1H).

### Example 49. Compound 51

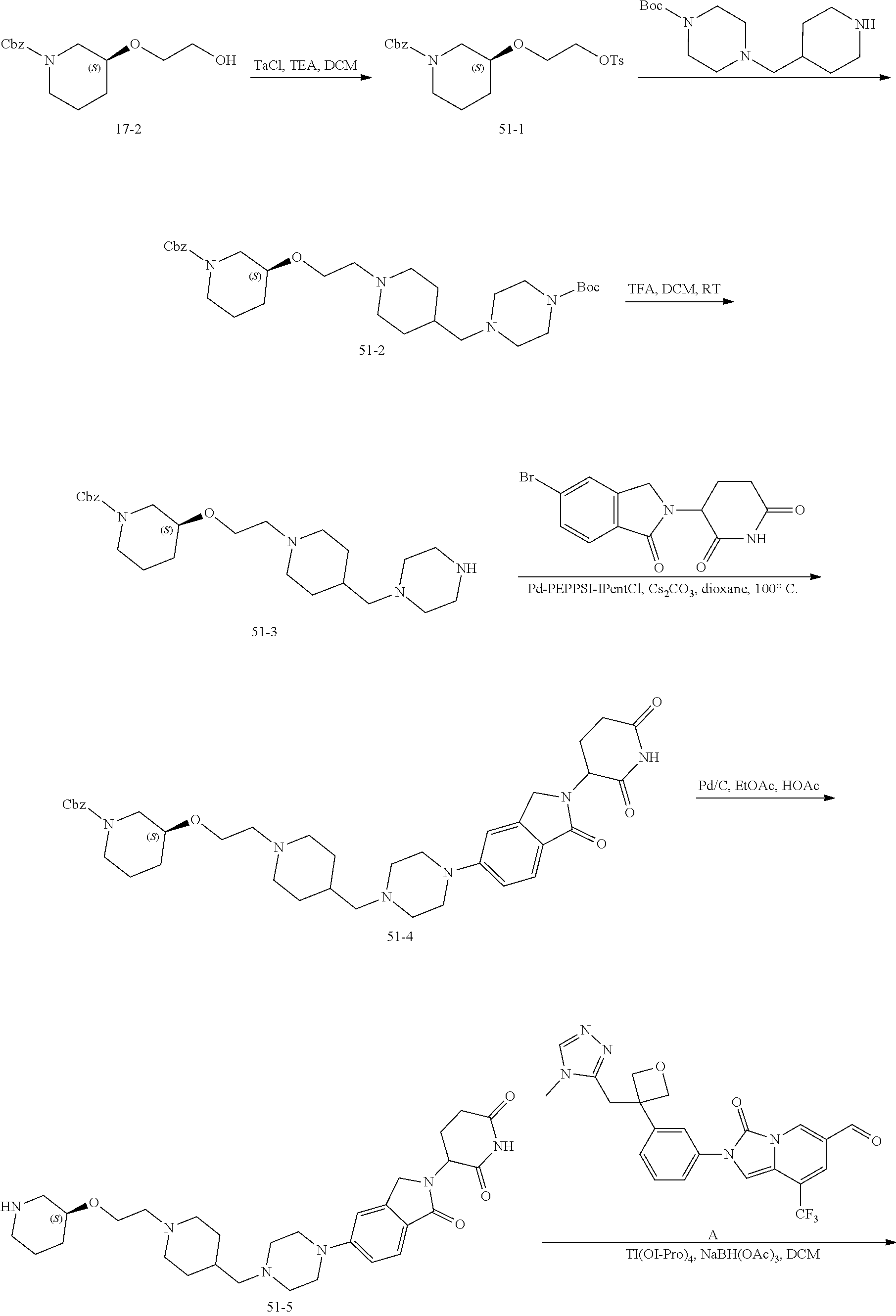

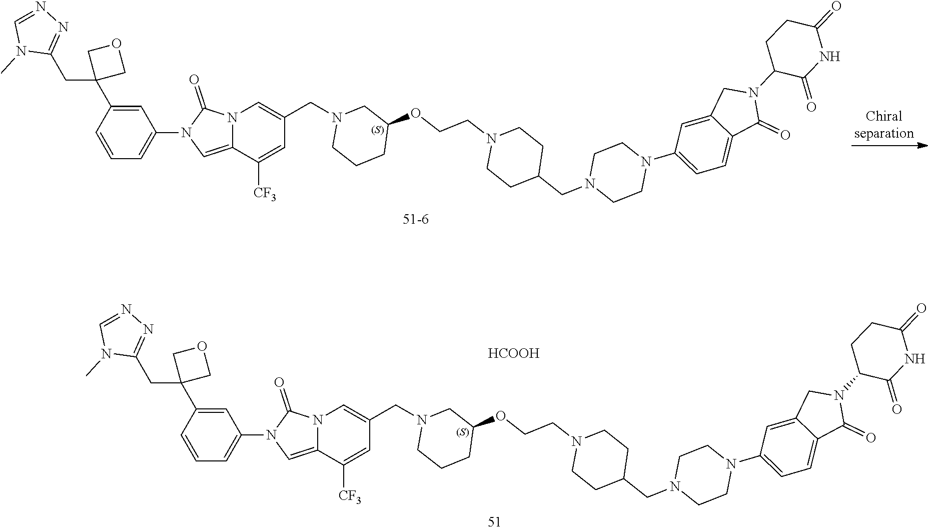

### Synthesis of Compound 51-1.

A solution of benzyl Compound 17-2 (20 g, 71.59 mmol, 10.0 equiv) and TEA (21.7 g, 214.79 mmol, 3.0 equiv), TsCl (20.5 g, 107.39 mmol, 1.5 equiv) in DCM (200 mL) was stirred for overnight at room temperature under nitrogen atmosphere. The reaction was quenched with sat. NH₄Cl (aq.) (400 mL) at room temperature. The aqueous layer was extracted with CH₂Cl₂ (6×200 mL). The resulting mixture was concentrated under vacuum. The residue was purified by silica gel column chromatography, eluted with PE/EA (5:1) to afford Compound 51-1 (15 g, 48%) as a yellow oil.

### Synthesis of Compound 51-2.

A solution of tert-butyl 4-(piperidin-4-ylmethyl) piperazine-1-carboxylate (9.8 g, 34.60 mmol, 1.0 equiv) in MeCN (150 mL) was treated with K₂CO₃ (19.1 g, 138.40 mmol, 4.0 equiv) for 30 min at room temperature under nitrogen atmosphere followed by the addition of Compound 51-1 (15 g, 34.60 mmol, 1 equiv) at 80° C. The resulting mixture was stirred for overnight at room temperature under nitrogen atmosphere. The reaction was quenched with water at room temperature. The aqueous layer was extracted with CH₂Cl₂ (6×100 mL). The resulting mixture was concentrated under vacuum. This resulted in Compound 51-2 (14 g, 74%) as a yellow oil.

### Synthesis of Compound 51-3.

A solution of Compound 51-2 (7.5 g, 13.78 mmol, 1.0 equiv) and TFA (25 mL) in DCM (75 mL) was stirred for 2 h at room temperature under nitrogen atmosphere. The resulting mixture was concentrated under vacuum. The residue was purified by reverse phase flash with the following conditions (Mobile Phase A: 10 mol/L NH₄CO₃ (aq.), Mobile Phase B: ACN; Flow rate: 100 mL/min; Gradient: 36% B to 64% B in 21 min; Wave Length: 220/254 nm) to afford Compound 51-3 (5 g, 82%) as a yellow solid.

### Synthesis of Compound 51-4.

To a stirred solution of Compound 51-3 (4 g, 8.99 mmol, 1.0 equiv) and 3-(5-bromo-1-oxo-3H-isoindol-2-yl) piperidine-2,6-dione (5.8 g, 17.99 mmol, 2.0 equiv) in 1,4-dioxane (40 mL) were added Cs₂CO₃ (8.8 g, 26.98 mmol, 3.0 equiv) and Pd-PEPPSI-IPentCl 2-methylpyridine (o-picoline) (378 mg, 0.45 mmol, 0.05 equiv) at room temperature under nitrogen atmosphere. The resulting mixture was stirred for overnight at 100° C. under nitrogen atmosphere. The reaction was quenched by the addition of 10% AcOH (300 mL) at room temperature. The resulting mixture was extracted with MeOH:CH₂Cl₂ (1:10) (5×200 mL). The resulting mixture was concentrated under reduced pressure. The residue was purified by silica gel column chromatography, eluted with CH₂Cl₂/MeOH (20:1) to afford Compound 51-4 (2.7 g, 37%) as an off-white solid.

### Synthesis of Compound 51-5.

To a solution of Compound 51-4 (2.7 g, 3.93 mmol, 1.0 equiv) in 80 mL EtOAc and 16 mL AcOH was added Pd/C (10%, 810 mg) under nitrogen atmosphere in a 250 mL round-bottom flask. The mixture was hydrogenated at room temperature for 2 h under hydrogen atmosphere using a hydrogen balloon, filtered through a Celite pad and concentrated under reduced pressure. This resulted in Compound 51-5 (2.1 g, 96%) as a colorless oil.

### Synthesis of Compound 51-6.

To a stirred solution of Compound 51-5 (800 mg, 1.44 mmol, 1.0 equiv) and Intermediate A (993 mg, 2.17 mmol, 1.5 equiv) in DCM (8 mL) was added Ti(Oi-Pr)₄ (1.6 g, 5.78 mmol, 4.0 equiv) at room temperature. The resulting mixture was stirred for overnight at room temperature. To the above mixture was added NaBH(OAc)₃ (920 mg, 4.34 mmol, 3.0 equiv) at room temperature. The resulting mixture was stirred for additional 2 h at room temperature. The reaction was quenched by the addition of 10% AcOH (100 mL) at room temperature. The aqueous layer was extracted with EtOAc (2×100 mL). The aqueous phase was concentrated under reduced pressure. The residue was purified by reversed-phase flash chromatography with the following conditions: column, C18 silica gel; mobile phase, MeCN in water (0.1% FA), 10% to 50% gradient in 30 min; UV 254 nm. This resulted in Compound 51-6 (240 mg, 17%) as a yellow solid.

### Synthesis of Compound 51.

The Compound 51-6 (240 mg) was purified by Prep-CHIRAL-HPLC with the following conditions (Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 60% B to 60% B in 25 min; Wave Length: 220/254 nm; RT1 (min): 12.32; RT2 (min): 19.03; Sample Solvent: DCM:ACN=1:1—HPLC; Injection Volume: 1.55 mL; Number of Runs: 3). The first peak was the product. The residue was purified by reversed-phase flash chromatography with the following conditions: column, (C18 silica gel; mobile phase, MeCN in water (0.1% FA), 10% to 50% gradient in 30 min; detector, UV 254 nm. This resulted in Compound 51 (76 mg, 30%) as a yellow solid.

LC-MS-Compound 51: (ES, m/z): [M-COOH]⁺ 994

H-NMR-Compound 51: (400 MHz, DMSO-d6, δ ppm): 1.07-1.13 (m, 3H), 1.31-1.41 (m, 2H), 1.63-1.66 (m, 3H), 1.90-2.01 (m, 6H), 2.12-2.14 (m, 3H), 2.31-2.33 (m, 1H), 2.45-2.51 (m, 5H), 2.56-2.61 (m, 3H), 2.87-2.90 (m, 4H), 2.97 (s, 3H), 3.25-3.31 (m, 4H), 3.36-3.38 (m, 2H), 3.49-3.53 (s, 5H), 4.18-4.22 (d, 1H), 4.30-4.35 (d, 1H), 4.90-4.94 (m, 4H), 4.96-5.04 (m, 1H), 6.68-6.90 (d, 1H), 7.01-7.05 (m, 3H), 7.32 (s, 1H), 7.38-7.41 (m, 2H), 7.50-7.53 (d, 1H), 7.68 (s, 1H), 7.52-7.77 (m, 1H), 8.17 (s, 1H), 8.20 (s, 1H), 10.95 (s, 1H).

### Example 50. Compound 52

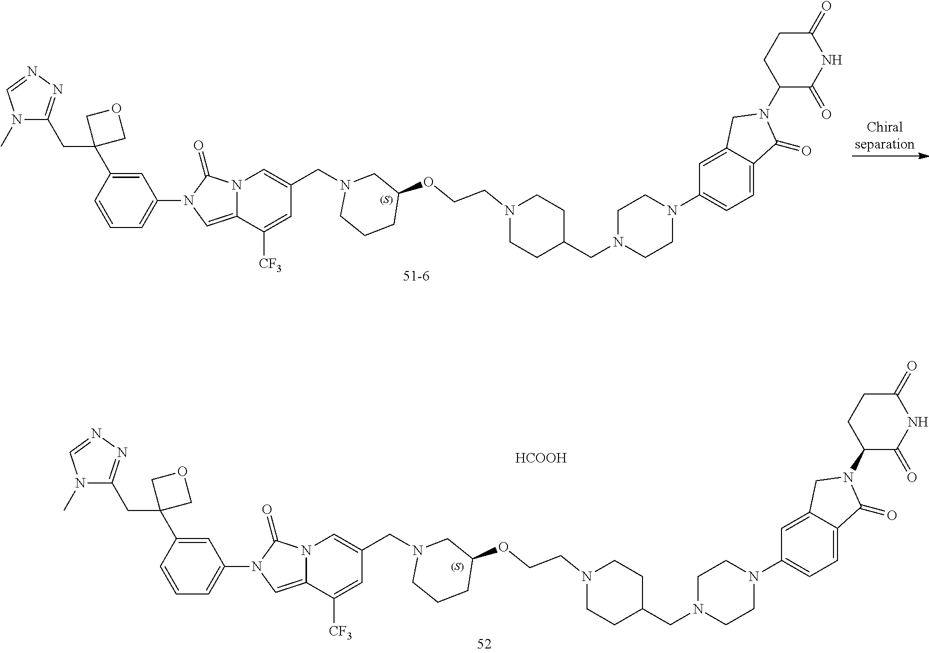

### Synthesis of Compound 52.

The Compound 51-6 (240 mg) was purified by Prep-CHIRAL-HPLC with the following conditions (Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 60% B to 60% B in 25 min; Wave Length: 220/254 nm; RT1 (min): 12.32; RT2 (min): 19.03; Sample Solvent: DCM:ACN=1:1—HPLC; Injection Volume: 1.55 mL; Number of Runs: 3). The second peak was the product. The residue was purified by reversed-phase flash chromatography with the following conditions: column, C18 silica gel; mobile phase, MeCN in water (0.1% FA), 10% to 50% gradient in 30 min; detector, UV 254 nm. This resulted in Compound 52 (75 mg, 30%) as a yellow solid.

LC-MS-Compound 52: (ES, m/z): [M-COOH]⁺ 994

H-NMR-Compound 52: (400 MHz, DMSO-d6, δ ppm): 1.07-1.13 (m, 3H), 1.31-1.41 (m, 2H), 1.63-1.66 (m, 3H), 1.90-2.01 (m, 6H), 2.12-2.14 (m, 3H), 2.31-2.33 (m, 1H), 2.45-2.51 (m, 5H), 2.56-2.61 (m, 3H), 2.87-2.90 (m, 4H), 2.97 (s, 3H), 3.25-3.31 (m, 4H), 3.36-3.38 (m, 2H), 3.49-3.53 (s, 5H), 4.18-4.22 (d, 1H), 4.30-4.35 (d, 1H), 4.90-4.94 (m, 4H), 4.96-5.04 (m, 1H), 6.68-6.90 (d, 1H), 7.01-7.05 (m, 3H), 7.32 (s, 1H), 7.38-7.41 (m, 2H), 7.50-7.53 (d, 1H), 7.68 (s, 1H), 7.52-7.77 (m, 1H), 8.17 (s, 1H), 8.20 (s, 1H), 10.95 (s, 1H).

### Example 51. Compound 53

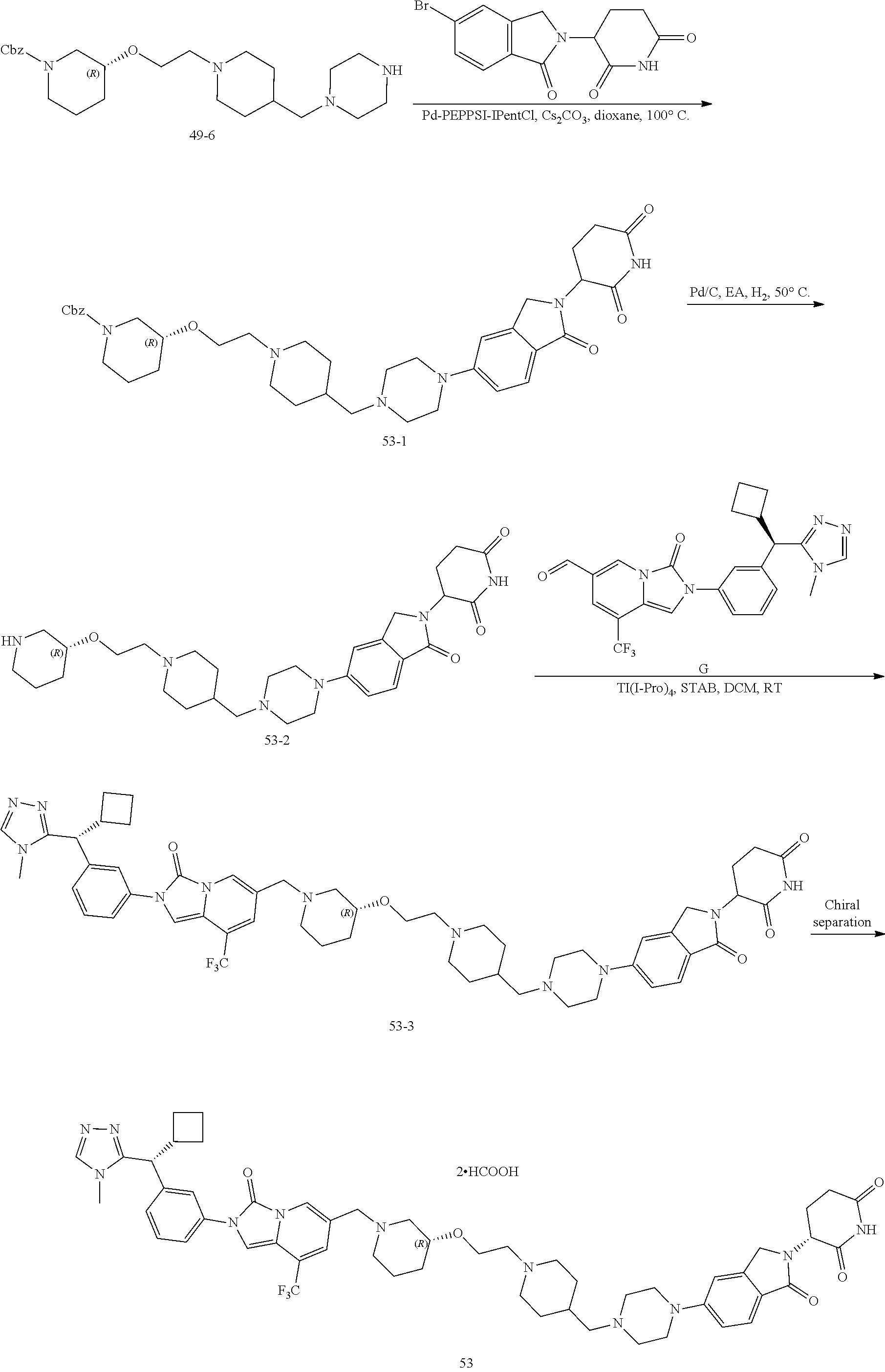

### Synthesis of Compound 53-1.

Into a 250 mL round-bottom flask were added Compound 49-6 (6 g, 13.495 mmol, 1 equiv), 3-(5-bromo-1-oxo-3H-isoindol-2-yl) piperidine-2,6-dione (4.36 g, 13.495 mmol, 1 equiv), Cs₂CO₃ (8.79 g, 26.990 mmol, 2 equiv), Pd-PEPPSI-IPentCl (0.57 g, 0.675 mmol, 0.05 equiv) and dioxane (60 mL) at room temperature. The resulting mixture was stirred for overnight at 100° C. under nitrogen atmosphere. The reaction was quenched by the addition of 10% AcOH (aq.) (450 mL) at room temperature. The aqueous layer was extracted with CH₂Cl₂ (3×300 mL). The resulting mixture was concentrated under reduced pressure. The residue was purified by silica gel column chromatography, eluted with CH₂Cl₂/MeOH (20:1) to afford Compound 53-1 (4 g, 41.00%) as an off-white solid.

### Synthesis of Compound 53-2.

To a solution of Compound 53-1 (1.6 g, 2.329 mmol, 1 equiv) in 30 mL EtOAc was added Pd/C (10%, 0.2 g) under nitrogen atmosphere in a 100 mL round-bottom flask. The mixture was hydrogenated at 50° C. for 2 h under hydrogen atmosphere using a hydrogen balloon, filtered through a Celite pad and concentrated under reduced pressure, to afford Compound 53-2 (1 g, 71.45%) as an off-white solid.

### Synthesis of Compound 53-3.

Into a 100 mL round-bottom flask were added Compound 53-2 (1 g, 1.809 mmol, 1 equiv), Intermediate G (1.24 g, 2.713 mmol, 1.50 equiv), titanium isopropylate (2.06 g, 7.236 mmol, 4.00 equiv) and DCM (10 mL) at room temperature. The resulting mixture was stirred for overnight at room temperature. To the above mixture was added STAB (0.77 g, 3.618 mmol, 2.00 equiv) at room temperature. The resulting mixture was stirred for 2 h at room temperature. The reaction was quenched by the addition of 10% AcOH (aq.) (10 mL) at room temperature. The aqueous layer was extracted with CH₂Cl₂ (3×20 mL). The resulting mixture was concentrated under reduced pressure. The residue was purified by Prep-TLC (CH₂Cl₂/MeOH 10:1) to afford Compound 53-3 (300 mg, 15.88%) as a yellow solid.

### Synthesis of Compound 53.

The Compound 53-3 (300 mg) was purified by Prep-Chiral-HPLC with the following conditions (Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 60% B to 60% B in 12 min; Wave Length: 220/254 nm; RT1 (min): 5.94; RT2 (min): 8.54; The first peak was product; Sample Solvent: DCM:ACN=1:1—HPLC; Injection Volume: 0.45 mL; Number of Runs: 11) to afford crude product. The crude product was purified by reversed-phase flash chromatography with the following conditions: column, 40 g C18 silica gel; mobile phase, MeCN in water (0.1% FA), 20% to 55% gradient in 8 min; detector, UV 254 nm to afford Compound 53 (65.2 mg, 20.78%) as a yellow solid.

LC-MS-Compound 53: (ES, m/z): [M−2HCOOH+H]⁺ 992

H-NMR-Compound 53: ((400 MHz, Methanol-d4, δ ppm): 1.43-1.59 (t, 2H), 1.60-1.84 (m, 4H), 1.85-2.00 (s, 6H), 2.01-2.23 (m, 4H), 2.31-2.41 (s, 2H), 2.41-2.53 (m, 1H), 2.54-2.68 (s, 5H), 2.70-2.84 (m, 3H), 2.86-3.08 (t, 3H), 3.20-3.32 (s, 3H), 3.35-3.45 (m, 4H), 3.46-3.58 (s, 4H), 3.59-3.74 (d, 4H), 3.74-3.91 (t, 2H), 4.27-4.33 (d, 1H), 4.33-4.49 (d, 1H), 5.08-5.16 (m, 1H), 7.04-7.13 (t, 3H), 7.15-7.19 (s, 1H), 7.28-7.34 (d, 1H), 7.46-7.53 (m, 1H), 7.66 (t, 2H), 7.76-7.82 (s, 1H), 8.37 (s, 1H).

### Example 52. Compound 54

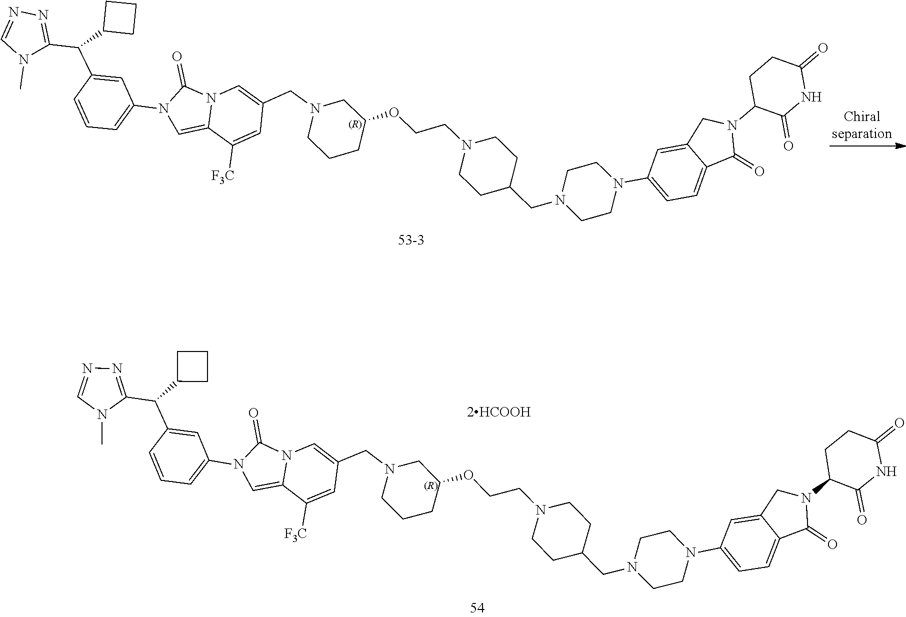

### Synthesis of Compound 54.

The Compound 53-3 (300 mg) was purified by Prep-Chiral-HPLC with the following conditions (Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 60% B to 60% B in 12 min; Wave Length: 220/254 nm; RT1 (min): 5.94; RT2 (min): 8.54; The second peak was the product; Sample Solvent: DCM:ACN=1:1—HPLC; Injection Volume: 0.45 mL; Number of Runs: 11) to afford crude product. The crude product was purified by reversed-phase flash chromatography with the following conditions: column, 40 g C18 silica gel; mobile phase, MeCN in water (0.1% FA), 20% to 55% gradient in 8 min; detector, UV 254 nm to afford Compound 54 (78.5 mg, 25.41%) as a yellow solid.

LC-MS-Compound 54: (ES, m/z): [M−2HCOOH+H]⁺ 992

H-NMR-Compound 54: ((400 MHz, CD₃OD-d4, δ ppm): 1.44-1.56 (t, 2H), 1.59-1.72 (s, 2H), 1.72-1.81 (t, 2H), 1.82-2.04 (s, 6H), 2.04-2.13 (t, 2H), 2.14-2.93 (t, 2H), 2.34 (d, 2H), 2.38-2.74 (s, 8H), 2.75-2.84 (d, 1H), 2.87-2.98 (t, 3H), 2.99-3.17 (s, 2H), 3.31 (s, 3H), 3.36 (d, 4H), 3.39-3.48 (d, 2H), 3.49-3.57 (s, 4H), 3.58-3.71 (d, 3H), 3.76-3.90 (t, 2H), 4.26-4.33 (d, 1H), 4.35-4.50 (d, 2H), 5.09-5.17 (m, 1H), 6.98-7.12 (d, 3H), 7.13-7.20 (s, 1H), 7.23-7.38 (d, 1H), 7.44-7.54 (m, 1H), 7.66 (t, 2H), 7.72-7.80 (s, 1H), 8.38 (s, 1H).

### Example 53. Compound 55

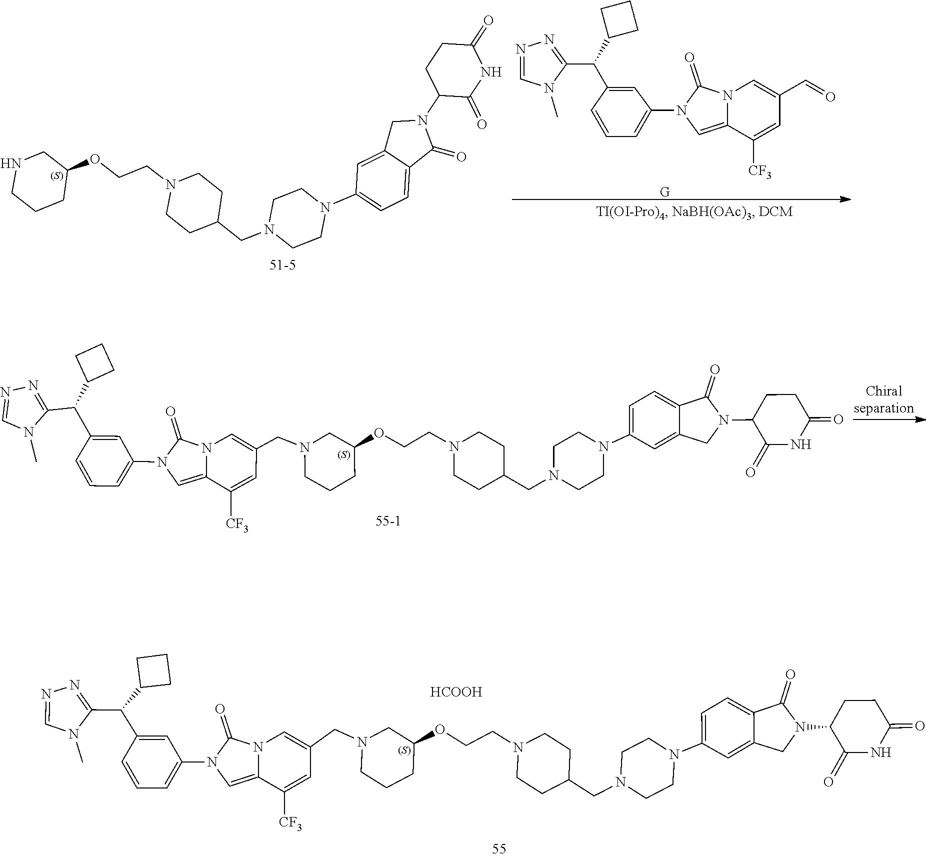

### Synthesis of Compound 55-1.

To a stirred solution of Compound 51-5 (0.800 mg, 0.144 mmol, 1.0 equiv) and Intermediate G (988 mg, 2.17 mmol, 1.5 equiv) in DCM (8 mL) was added Ti(Oi-Pr)₄ (1.6 g, 5.78 mmol, 4.0 equiv) at room temperature. The resulting mixture was stirred for overnight at room temperature. To the above mixture was added NaBH(OAc)₃ (920 mg, 4.34 mmol, 3.0 equiv) at room temperature. The resulting mixture was stirred for additional 2 h at room temperature. The reaction was quenched by the addition of 10% AcOH (100 mL) at room temperature. The aqueous layer was extracted with EtOAc (2×60 mL). The aqueous phase was concentrated under reduced pressure. The residue was purified by reversed-phase flash chromatography with the following conditions: column, C18 silica gel; mobile phase, MeCN in water (0.1% FA), 10% to 50% gradient in 30 min; detector, UV 254 nm. This resulted in Compound 55-1 (340 mg, 24%) as a yellow solid.

### Synthesis of Compound 55.

The Compound 55-1 (340 mg) was purified by Prep-CHIRAL-HPLC with the following conditions Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 60% B to 60% B in 11.5 min; Wave Length: 220/254 nm; RT1 (min): 5.06; RT2 (min): 6.96; Sample Solvent: DCM:ACN=1:1—HPLC; Injection Volume: 0.5 mL; Number of Runs: The first peak was the product. The residue was purified by reversed-phase flash chromatography with the following conditions: column, C18 silica gel; mobile phase, MeCN in water (0.1% FA), 10% to 50% gradient in 30 min. This resulted in Compound 55 (72 mg, 20%) as a yellow solid.

LC-MS-Compound 55: (ES, m/z): [(M−HCOOH)/2+H]⁺ 497

H-NMR-Compound 55: (400 MHz, CD₃OD δ ppm): 1.41-1.45 (m, 2H), 1.65-1.76 (m, 2H), 1.76-1.79 (m, 2H), 1.90-1.92 (m, 6H), 2.01-2.08 (m, 2H), 2.10-2.13 (m, 1H), 2.20-2.23 (m, 1H), 2.34-2.35 (m, 2H), 2.47-2.49 (m, 1H), 2.50-2.62 (m, 7H), 2.80-2.81 (m, 1H), 2.85-2.92 (m, 1H), 3.01-3.05 (m, 2H), 3.28-3.32 (m, 1H), 3.34-3.35 (m, 5H), 3.43-3.50 (m, 5H), 3.55-3.64 (m, 3H), 3.80-3.83 (m, 2H), 4.28-4.30 (m, 2H), 4.41-4.42 (m, 2H), 5.10-5.14 (m, 1H), 7.07-7.09 (m, 3H), 7.16 (s, 1H), 7.30-7.32 (m, 1H), 7.48-7.52 (m, 1H), 7.64-7.66 (m, 3H), 7.76 (s, 1H), 8.35-8.38 (m, 3H).

### Example 54. Compound 56

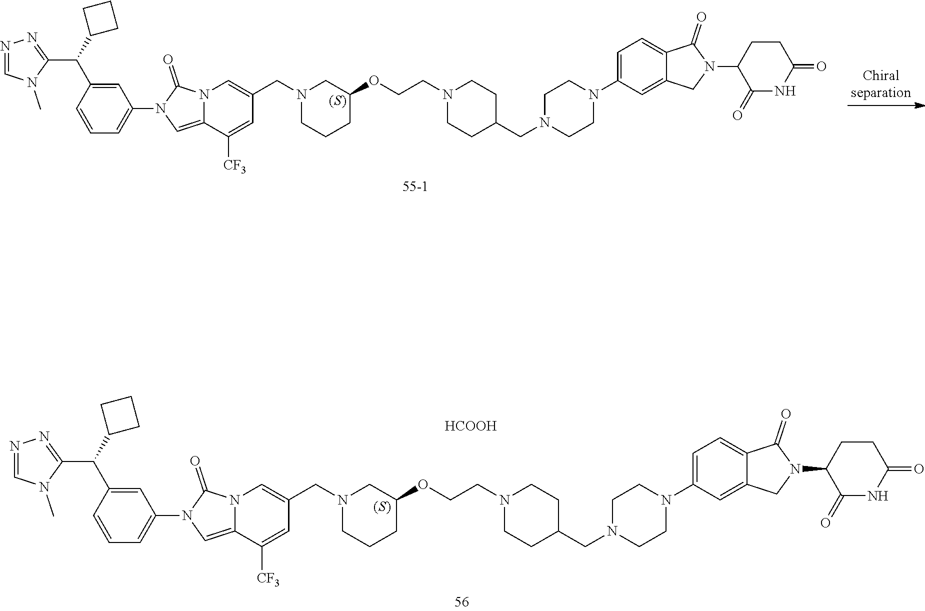

### Synthesis of Compound 56.

The Compound 55-1 (340 mg) was purified by Prep-CHIRAL-HPLC with the following conditions Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 60% B to 60% B in 11.5 min; Wave Length: 220/254 nm; RT1 (min): 5.06; RT2 (min): 6.96; Sample Solvent: DCM:ACN=1:1—HPLC; Injection Volume: 0.5 mL; Number of Runs: The second peak was the product. The residue was purified by reversed-phase flash chromatography with the following conditions: column, C18 silica gel; mobile phase, MeCN in water (0.1% FA), 10% to 50% gradient in 30 min. This resulted in Compound 56 (98 mg, 28%) as a yellow solid.

LC-MS-Compound 56: (ES, m/z): [(M−HCOOH)/2+H]⁺ 497

H-NMR-Compound 56: (400 MHz, CD₃OD δ ppm): 1.41-1.45 (m, 2H), 1.65-1.76 (m, 2H), 1.76-1.79 (m, 2H), 1.90-1.92 (m, 6H), 2.01-2.08 (m, 2H), 2.10-2.13 (m, 1H), 2.20-2.23 (m, 1H), 2.34-2.35 (m, 2H), 2.47-2.49 (m, 1H), 2.50-2.62 (m, 7H), 2.80-2.81 (m, 1H), 2.85-2.92 (m, 1H), 3.01-3.05 (m, 2H), 3.28-3.32 (m, 1H), 3.34-3.35 (m, 5H), 3.43-3.50 (m, 5H), 3.55-3.64 (m, 3H), 3.80-3.83 (m, 2H), 4.28-4.30 (m, 2H), 4.41-4.42 (m, 2H), 5.10-5.14 (m, 1H), 7.07-7.09 (m, 3H), 7.16 (s, 1H), 7.30-7.32 (m, 1H), 7.48-7.52 (m, 1H), 7.64-7.66 (m, 3H), 7.76 (s, 1H), 8.35-8.38 (m, 3H).

### Example 55. Compound 57

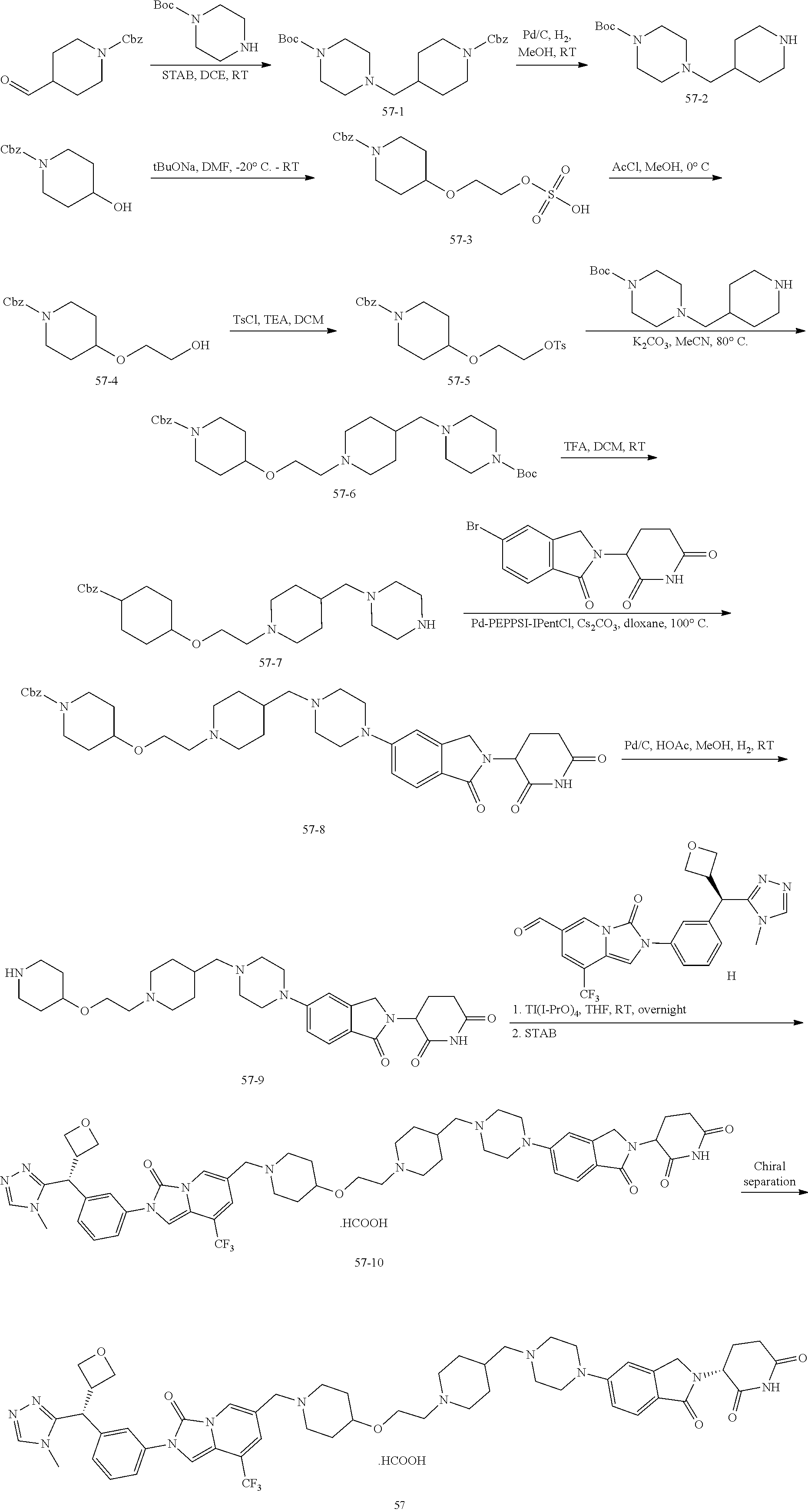

### Synthesis of Compound 57-1.

To a stirred solution of benzyl 4-formylpiperidine-1-carboxylate (50 g, 202.188 mmol, 1 equiv) and tert-butyl piperazine-1-carboxylate (37.66 g, 202.188 mmol, 1 equiv) in DCE (100 mL) were added STAB (85.70 g, 404.376 mmol, 2 equiv) at room temperature. The resulting mixture was stirred for overnight at room temperature. The reaction was quenched with water at room temperature. The aqueous layer was extracted with DCM (2×200 mL). The resulting mixture was concentrated under vacuum. The residue was purified by silica gel column chromatography, eluted with PE/EA (50:1) to afford Compound 57-1 (75 g, 79.95%) as an off-white solid.

### Synthesis of Compound 57-2.

To a solution of Compound 57-1 (25 g, 59.873 mmol, 1 equiv) in MeOH (150 mL) was added Pd/C (2.5 g, 10%) under nitrogen atmosphere in a 500 mL round-bottom flask. The mixture was hydrogenated at room temperature for 3 h under hydrogen atmosphere using a hydrogen balloon, filtered through a Celite pad and concentrated under reduced pressure. The crude product was used in the next step directly without further purification. This resulted in Compound 57-2 (16 g, 84.86%) as a light brown solid.

### Synthesis of Compound 57-3.

To a stirred solution of benzyl 4-hydroxypiperidine-1-carboxylate (50 g, 212.510 mmol, 1 equiv) and 1,3,2lambda6-dioxathiolane-2,2-dione (65.94 g, 531.275 mmol, 2.5 equiv) in DMF (500 mL) was added sodium 2-methylpropan-2-olate (50.04 g, 520.649 mmol, 2.45 equiv) in portions at −20° C. under nitrogen atmosphere. The resulting mixture was stirred overnight at room temperature under nitrogen atmosphere. The reaction was quenched by the addition of water (1.5 L) at room temperature. The aqueous layer was extracted with DCM (3×500 mL). The aqueous layer was concentrated under reduced pressure. This resulted in Compound 57-3 (60 g, 70.70%) as a yellow oil.

### Synthesis of Compound 57-4.

To a stirred solution of Compound 57-3 (60 g, 166.950 mmol, 1 equiv) in MeOH (700 mL) was added AcCl (100 mL, 1401.274 mmol, 8.39 equiv) dropwise at 0° C. The resulting mixture was stirred for overnight at room temperature. The reaction was basified to pH 7 with NaHCO₃ (aq.) at room temperature. The aqueous layer was extracted with MTBE (3×500 mL) and dried over anhydrous Na₂SO₄. After filtration, the filtrate was concentrated under reduced pressure. The crude product was used in the next step directly without further purification. This resulted in Compound 57-4 (30 g, 57.90%) as a light yellow oil.

### Synthesis of Compound 57-5.

To a stirred solution of Compound 57-4 (30 g, 107.398 mmol, 1 equiv) and TEA (32.60 g, 322.194 mmol, 3 equiv) in DCM (450 mL) was added TsCl (51.19 g, 268.495 mmol, 2.50 equiv) at room temperature. The resulting mixture was stirred overnight at room temperature. The reaction was quenched with water (500 mL) at room temperature. The aqueous layer was extracted with DCM (2×300 mL). The resulting mixture was concentrated under vacuum. The residue was purified by silica gel column chromatography, eluted with DCM/MeOH (100:1) to afford Compound 57-4 (30 g, 57.99%) as a light yellow oil.

### Synthesis of Compound 57-6.

To a stirred solution of Compound 57-5 (18 g, 41.521 mmol, 1 equiv) and Compound 57-2 (14.71 g, 51.903 mmol, 1.25 equiv) in MeCN (300 mL) was added K₂CO₃ (22.95 g, 166.058 mmol, 4.00 equiv) at room temperature. The resulting mixture was stirred overnight at 80° C. The resulting mixture was diluted with water (500 mL). The aqueous layer was extracted with DCM (2×200 mL). The resulting mixture was concentrated under vacuum. The residue was purified by silica gel column chromatography, eluted with DCM/MeOH (50:1) to afford Compound 57-6 (18 g, 71.63%) as a light yellow oil.

### Synthesis of Compound 57-7.

A solution of Compound 57-6 (18 g, 33.043 mmol, 1 equiv) and TFA (11.30 g, 99.129 mmol, 3 equiv) in DCM (180 mL) was stirred for 2 h at room temperature. The resulting mixture was concentrated under reduced pressure. The residue was purified by reverse flash chromatography with the following conditions: column, C18 silica gel; mobile phase, MeCN in Water (10 mmol/L NH₄HCO₃), 40% to 80% gradient in 30 min; detector, UV 220 nm. This resulted in Compound 57-7 (12 g, 73.51%) as a yellow solid.

### Synthesis of Compound 57-8.

To a stirred mixture of Compound 57-7 (10 g, 22.491 mmol, 1 equiv) and 3-(5-bromo-1-oxo-3H-isoindol-2-yl)piperidine-2,6-dione (14.54 g, 44.982 mmol, 2 equiv) in dioxane (200 mL) were added Cs₂CO₃ (21.98 g, 67.473 mmol, 3 equiv) and Pd-PEPPSI-IPentCl 2-methylpyridine (o-picoline) (1.89 g, 2.249 mmol, 0.1 equiv) at room temperature under nitrogen atmosphere. The resulting mixture was stirred overnight at 100° C. under nitrogen atmosphere. The mixture was allowed to cool down to room temperature and acidified to pH 6 with HOAc. The mixture was concentrated under reduced pressure. The residue was purified by silica gel column chromatography, eluted with DCM (0.1% HOAc)/MeOH (8:1) to afford Compound 57-7 (6.5 g, 38.29%) as a brown solid.

### Synthesis of Compound 57-9.

To a solution of Compound 57-8 (6.5 g, 9.463 mmol, 1 equiv) and HOAc (1 mL) in MeOH (150 mL) was added Pd/C (0.65 g, 10%) under nitrogen atmosphere in a 500 mL round-bottom flask. The mixture was hydrogenated at room temperature for 3 h under hydrogen atmosphere using a hydrogen balloon, filtered through a Celite pad and concentrated under reduced pressure. The crude product was used in the next step directly without further purification. This resulted in Compound 57-8 (5 g, 86.03%) as a light brown solid.

### Synthesis of Compound 57-10.

To a stirred solution of Intermediate H (500 mg, 1.093 mmol, 1 equiv) and Compound 57-9 (543.76 mg, 0.984 mmol, 0.9 equiv) in THF (30 mL) was added Ti(Oi-Pr)₄ (932.04 mg, 3.279 mmol, 3 equiv) at room temperature. The resulting mixture was stirred overnight at room temperature. To the above mixture was added STAB (695.02 mg, 3.279 mmol, 3 equiv) at room temperature. The resulting mixture was stirred for additional 4 h at room temperature. The reaction was quenched with 10% HOAc(aq.) (200 mL) at room temperature. The aqueous layer was extracted with EtOAc (80 mL). The aqueous layer was filtered, the filter cake was washed with water (10 mL). The filtrate was concentrated under reduced pressure. The crude product was purified by Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 4% B to 18% B in 7 min, Wave Length: 254; 220 nm; RT1 (min): 6.05) to afford Compound 57-10 (205 mg, 17.67%) as a yellow solid.

### Synthesis of Compound 57.

The Compound 57-10 (205 mg) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 60% B to 60% B in 15 min; Wave Length: 220/254 nm; RT1 (min): 7.33; RT2 (min): 11.00; the first peak is product) to afford the crude product. The residue was purified by reverse flash chromatography with the following conditions: column, C18 silica gel; mobile phase, MeCN in water (0.1% FA), 10% to 50% gradient in 10 min; detector, UV 254 nm. This resulted in Compound 57 (29.5 mg, 14.13%) as a yellow solid.

LC-MS-Compound 57: (ES, m/z): [M−HCOOH+H]⁺ 995

H-NMR-Compound 57: (400 MHz, DMSO-d6, δ ppm): 1.12-1.21 (m, 2H), 1.43-1.45 (m, 2H), 1.48-1.65 (m, 1H), 1.69-1.72 (m, 2H), 1.81-1.89 (m, 2H), 1.91-2.02 (m, 1H), 2.08-2.21 (m, 6H), 2.31-2.50 (m, 5H), 2.51-2.71 (m, 5H), 2.85-3.05 (m, 3H), 3.21-3.31 (m, 6H), 3.43 (s, 3H), 3.52-3.55 (m, 2H), 3.84-3.95 (m, 1H), 4.18-4.28 (m, 3H), 4.48-4.50 (m, 2H), 4.72-4.81 (m, 2H), 5.03-5.13 (d, 1H), 7.01-7.07 (m, 3H), 7.18-7.20 (d, 1H), 7.39 (s, 1H), 7.49-7.53 (m, 2H), 7.67 (s, 1H), 7.75-7.77 (m, 2H), 8.17 (s, 1H), 8.38 (s, 1H), 10.95 (s, 1H).

### Example 56. Compound 58

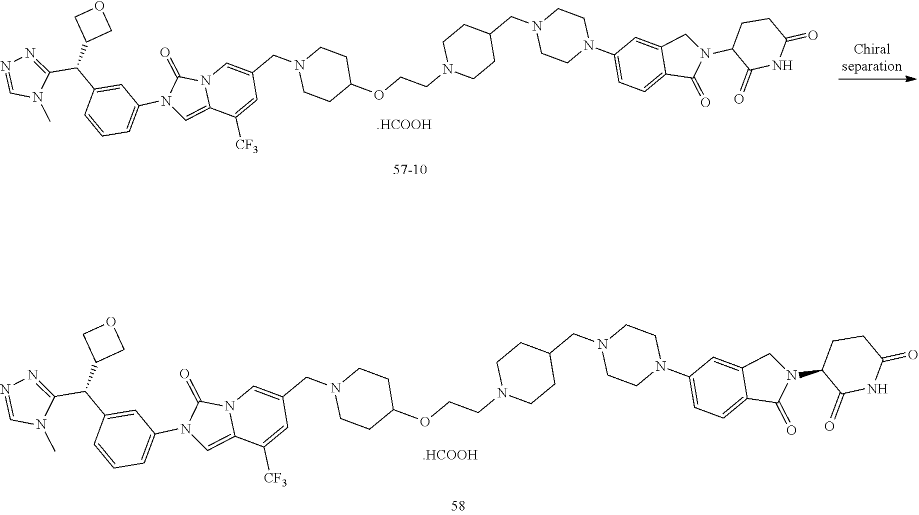 [SMILES: Cn1cnnc1[C@@H](c1cccc(-n2cc3c(C(F)(F)F)cc(CN4CCC(OCCN5CCC(CN6CCN(c7ccc8c(c7)CN(C7CCC(=O)NC7=O)C8=O)CC6)CC5)CC4)cn3c2=O)c1)C1COC1 ; ".HCOOH" ; Cn1cnnc1[C@@H](c1cccc(-n2cc3c(C(F)(F)F)cc(CN4CCC(OCCN5CCC(CN6CCN(c7ccc8c(c7)CN([C@H]7CCC(=O)NC7=O)C8=O)CC6)CC5)CC4)cn3c2=O)c1)C1COC1 ; ".HCOOH"]

### Synthesis of Compound 58.

The Compound 57-10 (205 mg) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 60% B to 60% B in 15 min; Wave Length: 220/254 nm; RT1 (min): 7.33; RT2 (min): 11.00; the second peak is product) to afford Compound 58 (42.7 mg, 20.73%) as a yellow solid.

LC-MS-Compound 58: (ES, m/z): [M−HCOOH+H]⁺ 995

H-NMR-Compound 58: (400 MHz, DMSO-d6, δ ppm): 1.12-1.21 (m, 2H), 1.43-1.45 (m, 2H), 1.48-1.65 (m, 1H), 1.69-1.72 (m, 2H), 1.81-1.89 (m, 2H), 1.91-2.02 (m, 1H), 2.08-2.21 (m, 6H), 2.31-2.39 (m, 1H), 2.41-2.50 (m, 4H), 2.51-2.71 (m, 5H), 2.85-3.05 (m, 3H), 3.21-3.31 (m, 6H), 3.43 (s, 3H), 3.52-3.55 (m, 2H), 3.84-3.95 (m, 1H), 4.18-4.35 (m, 3H), 4.48-4.50. (d, 2H), 4.72-4.81 (m, 2H), 5.03-5.13 (d, 1H), 7.01-7.07 (m, 3H), 7.18-7.20 (d, 1H), 7.39 (s, 1H), 7.49-7.53 (m, 2H), 7.67 (s, 1H), 7.75-7.77 (m, 2H), 8.18 (s, 2H), 8.38 (s, 1H), 10.95 (s, 1H).

### Example 57. Compound 59

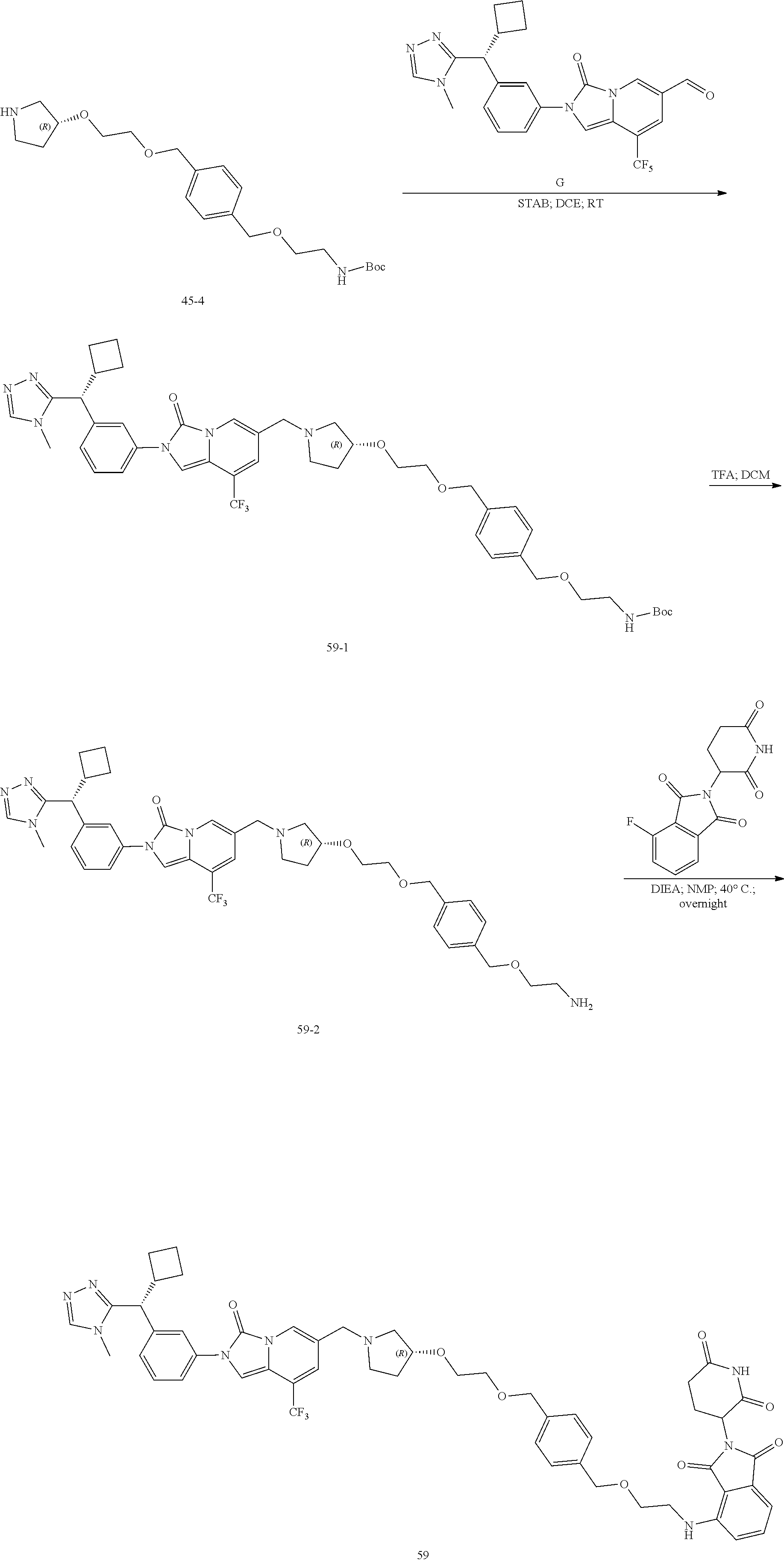

### Synthesis of Compound 59-1.

Into a 100 mL 3-necked round-bottom flask were added Compound 45-4 (2 g, 5.070 mmol, 1 equiv), DCE (25 mL), Intermediate G (2.31 g, 5.070 mmol, 1 equiv) and STAB (2.15 g, 10.140 mmol, 2 equiv) at room temperature. The resulting mixture was stirred for overnight at room temperature. The reaction was quenched by the addition of water (30 mL) at room temperature. The aqueous layer was extracted with EtOAc (3×20 mL). The resulting mixture was concentrated under reduced pressure. The residue was purified by reversed-phase flash chromatography with the following conditions: column, 120 g C18 silica gel; mobile phase, MeCN in Water (10 mmol/L NH₄HCO₃), 10% to 50% gradient in 12 min; detector, UV 254 nm to afford Compound 59-1 (800 mg, 18.92%) as a light yellow solid.

### Synthesis of Compound 59-2.

Into a 100 mL round-bottom flask were added Compound 59-1 (780 mg, 0.935 mmol, 1 equiv), DCM (9 mL) and TFA (3 mL) at room temperature. The resulting mixture was stirred for 2 h at room temperature. The resulting mixture was concentrated under reduced pressure. The residue was purified by Prep-TLC (CH₂Cl₂/MeOH 10:1) to afford Compound 59-2 (500 mg, 72.85%) as a light brown solid.

### Synthesis of Compound 59.

Into a 100 mL round-bottom flask were added Compound 59-2 (450 mg, 0.613 mmol, 1 equiv), NMP (5 mL), 2-(2,6-dioxopiperidin-3-yl)-4-fluoroisoindole-1,3-dione (508.15 mg, 1.839 mmol, 3 equiv) and DIEA (79.26 mg, 0.613 mmol, 1 equiv) at room temperature. The resulting mixture was stirred for overnight at 40° C. The crude product (5 mL) was purified by Prep-HPLC with the following conditions (Column: XBridge Prep OBD C18 Column, 30*150 mm, 5 μm; Mobile Phase A: water (10 mmol/L FA), Mobile Phase B: ACN; Flow rate: 60 mL/min; Gradient: 44% B to 64% B in 8 min, 64% B; Wave Length: 254 nm; RT1 (min): 7.28) to afford Compound 59 (28.9 mg, 4.55%) as a light yellow solid.

LC-MS-Compound 59: (ES, m/z): [M−HCOOH+H]⁺ 990

H-NMR-Compound 59: 1H NMR (300 MHz, CD₃OD-d4 ppm) δ 1.89-1.92 (m, 7H), 2.01-2.31 (m, 3H), 2.60-2.92 (m, 7H), 3.41-3.43 (m, 4H), 3.44-3.46 (m, 3H), 3.47-3.51 (m, 4H), 3.53-3.57 (m, 2H), 4.12 (s, 1H), 4.30-4.60 (m, 5H), 5.08-5.11 (m, 1H), 6.98-7.08 (d, 2H), 7.12 (s, 2H), 7.27-7.31 (m, 1H), 7.33-7.42 (m, 4H), 7.50-7.60 (m, 2H), 7.62-7.73 (m, 3H).

### Example 58. Compound 60

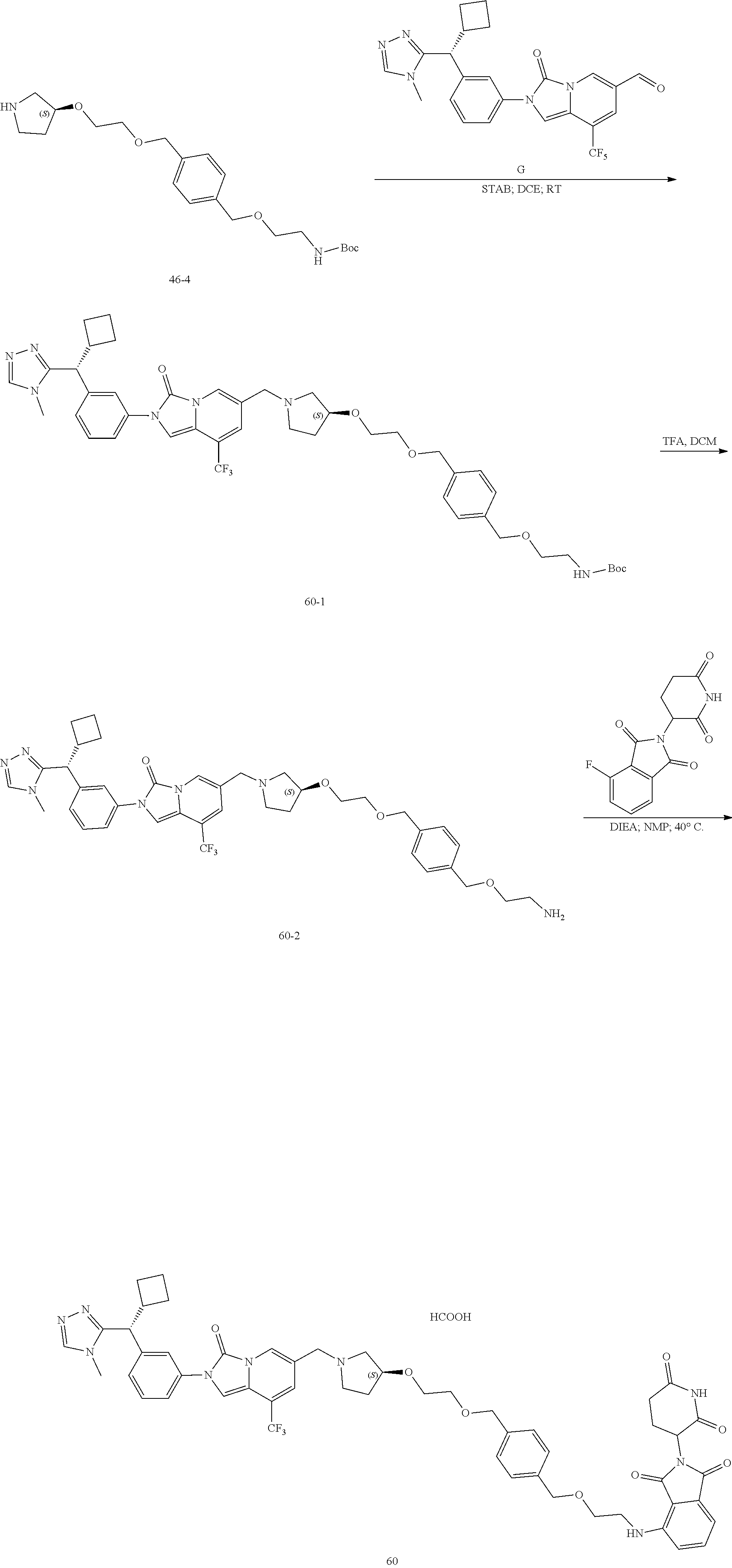

### Synthesis of Compound 60-1.

A solution of Compound 46-4 (2 g, 5.070 mmol, 1 equiv) and Intermediate G (2.31 g, 5.070 mmol, 1 equiv) in DCE (20 mL) was stirred for 1 h at room temperature. To the above mixture was added STAB (2.15 g, 10.140 mmol, 2 equiv) and AcOH (0.30 g, 5.070 mmol, 1 equiv) at room temperature. The resulting mixture was stirred for 2 h at room temperature. The resulting mixture was concentrated under reduced pressure. The residue was purified by Prep-TLC (CH₂Cl₂/MeOH 10:1) to afford Compound 60-1 (800 mg, 17.98%) as a yellow solid.

### Synthesis of Compound 60-2.

To a stirred mixture of TFA (3 mL) in DCM (9 mL) was added Compound 60-1 (800 mg, 0.957 mmol, 1 equiv) in portions at room temperature. The resulting mixture was stirred for 1 h at room temperature. The resulting mixture was concentrated under reduced pressure. The residue was purified by Prep-TLC (CH₂Cl₂/MeOH 10:1) to afford Compound 60-2 as a yellow solid.

### Synthesis of Compound 60.

Into a 100 mL round-bottom flask were added Compound 60-2 (400 mg, 0.545 mmol, 1 equiv), NMP (4.5 mL), 2-(2,6-dioxopiperidin-3-yl)-4-fluoroisoindole-1,3-dione (451.69 mg, 1.635 mmol, 3 equiv) and DIEA (70.45 mg, 0.545 mmol, 1 equiv) at room temperature. The resulting mixture was stirred for overnight at 40° C. The crude product (4.5 ml) was purified by Prep-HPLC with the following conditions (Column: Xselect CSH (C18 OBD Column 30*150 mm 5 μm, n; Mobile Phase A: Water (0.1% FA), Mobile Phase B: ACN; Flow rate: 60 mL/min; Gradient: 26% B to 37% B in 9 min, 37% B; Wave Length: 254; 220 nm; RT1 (min): 8.25) to afford Compound 60 (28.4 mg, 5.03%) as a light yellow solid.

LC-MS-Compound 60: (ES, m/z): [M−HCOOH+H]⁺ 990

H-NMR-Compound 60: (400 MHz, Methanol-d6, δ ppm): 1.75-2.28 (m, 1OH), 2.53-2.81 (m, 6H), 2.83-2.90 (t, 1H), 3.37-3.49 (m, 3H), 3.50 (t, 1H), 3.51-3.56 (m, 3H), 3.60-3.73 (t, 3H), 4.08-4.16 (s, 1H), 4.26-4.33 (d, 2H), 4.45-4.65 (m, 4H), 5.05-5.11 (m, 1H), 6.99-7.06 (d, 2H), 7.11 (s, 2H), 7.24-7.28 (d, 1H), 7.29-7.41 (m, 4H), 7.47-7.53 (t, 3H), 7.59-7.74 (m, 3H), 8.45-8.40 (s, 1H).

### Example 59. Compound 61

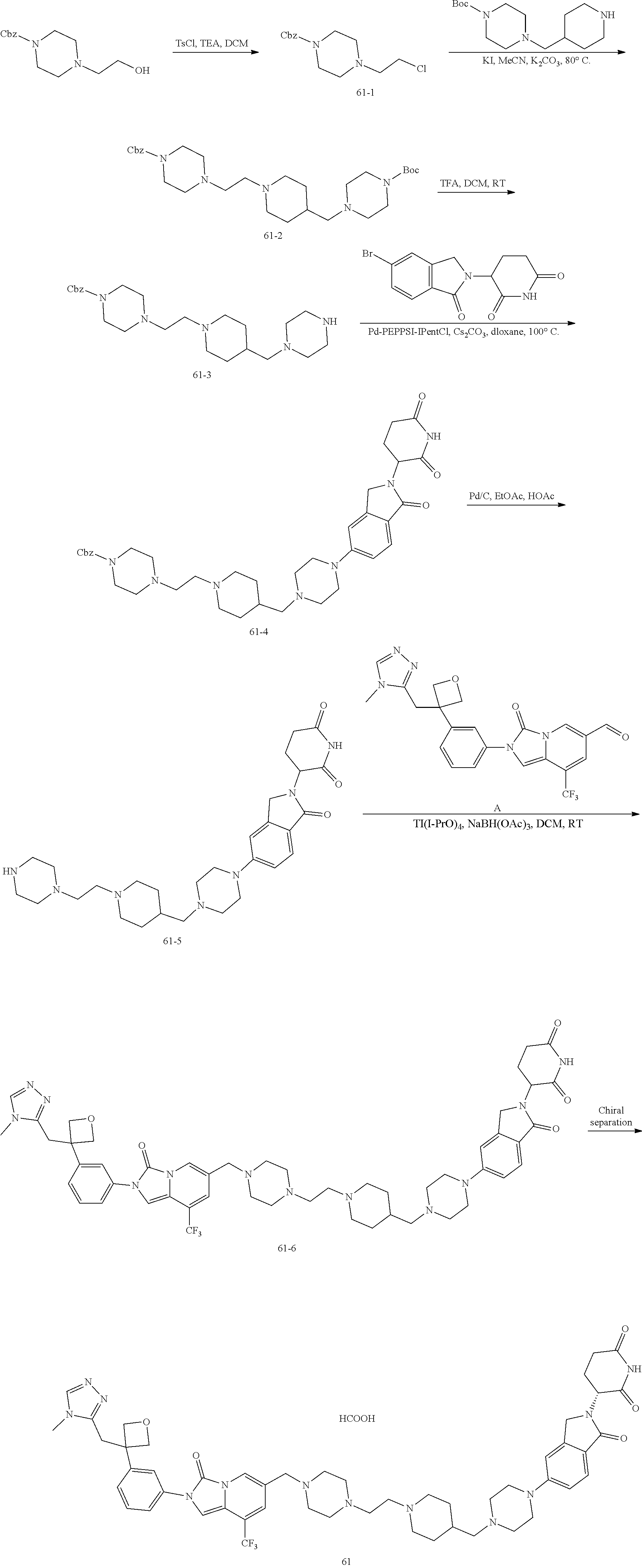

### Synthesis of Compound 61-1.

To a stirred mixture of benzyl 4-(2-hydroxyethyl) piperazine-1-carboxylate (20 g, 75.66 mmol, 1.0 equiv) and TEA (11.5 g, 113.49 mmol, 1.5 equiv) in DCM (200 mL) was added TsCl (28.8 g, 151.32 mmol, 2.0 equiv) at room temperature. The resulting mixture was stirred for overnight at room temperature. The reaction was quenched by the addition of water (200 mL) at room temperature. The aqueous layer was extracted with EtOAc (3×200 mL). The resulting mixture was concentrated under reduced pressure. The residue was purified by silica gel column chromatography, eluted with PE/EA (10:1) to afford Compound 61-1 (13.5 g, 41%) as an off-white oil.

### Synthesis of Compound 61-2.

To a stirred mixture of Compound 61-1 (13.5 g, 32.25 mmol, 1.0 equiv) and tert-butyl 4-(piperidin-4-ylmethyl)piperazine-1-carboxylate (14 g, 48.38 mmol, 1.5 equiv) in MeCN (150 mL) were added KI (5.4 g, 32.25 mmol, 1.0 equiv) and K₂CO₃ (8.9 g, 64.51 mmol, 2.0 equiv) at room temperature. The resulting mixture was stirred for overnight at 80° C. The reaction was quenched by the addition of water (150 mL) at room temperature. The aqueous layer was extracted with EtOAc (3×200 mL). The resulting mixture was concentrated under reduced pressure. The residue was purified by silica gel column chromatography, eluted with CH₂Cl₂/MeOH (100:1) to afford Compound 61-2 (18 g, 95%) as a yellow oil.

### Synthesis of Compound 61-3.

To a solution of Compound 61-2 (9.8 g, 18.50 mmol, 1.0 equiv) in 300 mL MeOH was added Pd/C (10%, 2 g) under nitrogen atmosphere in a 500 mL round-bottom flask. The mixture was hydrogenated at 50° C. for 2 h under hydrogen atmosphere using a hydrogen balloon, filtered through a Celite pad and concentrated under reduced pressure to afford Compound 61-3 (7.2 g, 93%) as a yellow oil.

### Synthesis of Compound 61-4.

To a stirred solution of Compound 61-3 (1 g, 2.32 mmol, 1.0 equiv) and 3-(5-bromo-1-oxo-3H-isoindol-2-yl) piperidine-2,6-dione (1.50 g, 4.65 mmol, 2.0 equiv) in 1,4-dioxane (10 mL) were added Cs₂CO₃ (2.28 g, 6.98 mmol, 3.0 equiv) and Pd-PEPPSI-IPentCl 2-methylpyridine (o-picoline) (0.1 g, 0.11 mmol, 0.05 equiv) at room temperature under nitrogen atmosphere. The resulting mixture was stirred for overnight at 100° C. under nitrogen atmosphere. The reaction was quenched by the addition of 10% AcOH (100 mL) at room temperature. The aqueous phase was concentrated under reduced pressure. The residue was purified by Prep-TLC (CH₂Cl₂/MeOH=10:1) to afford Compound 61-4 (600 mg, 38%) as an off-white solid.

### Synthesis of Compound 61-5.

To a solution of Compound 61-4 (600 mg, 0.89 mmol, 1.0 equiv) in 15 mL EtOAc and 3 mL AcOH was added Pd/C (10%, 200 mg) under nitrogen atmosphere in a 50 mL round-bottom flask. The mixture was hydrogenated at room temperature for 2 h under hydrogen atmosphere using a hydrogen balloon, filtered through a Celite pad and concentrated under reduced pressure. This resulted in Compound 61-5 (400 mg, 83%) as an off-white solid.

### Synthesis of Compound 61-6.

To a stirred solution of Compound 61-5 (1 g, 1.86 mmol, 1.0 equiv) and Intermediate A (1.3 g, 2.79 mmol, 1.5 equiv) in DCM (10 mL) was added Ti(Oi-Pr)₄ (2.1 g, 7.44 mmol, 4.0 equiv) at room temperature. The resulting mixture was stirred for overnight at room temperature. To the above mixture was added NaBH(OAc)₃ (1.2 g, 5.58 mmol, 3.0 equiv) at room temperature. The resulting mixture was stirred for additional 2 h at room temperature. The reaction was quenched by the addition of 10% AcOH (100 mL) at room temperature. The aqueous layer was extracted with EtOAc (2×60 mL). The aqueous phase was concentrated under reduced pressure. The crude product (500 mg) was purified by Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 3% B to 16% B in 7 min, 16% B; Wave Length: 254; 220 nm; RT1 (min): 6.5) to afford Compound 61-6 (280 mg, 15%) as a yellow solid.

### Synthesis of Compound 61.

The Compound 61-6 (330 mg) was purified by Prep-CHIRAL-HPLC with the following conditions Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 60% B to 60% B in 40 min; Wave Length: 220/254 nm; RT1 (min): 16.64; RT2 (min): 20.05; Sample Solvent: DCM:ACN=1:1—HPLC; Injection Volume: 1.5 mL; Number of Runs: 5. The first peak was the product. The residue was purified by reversed-phase flash chromatography with the following conditions: column, C18 silica gel; mobile phase, MeCN in water (0.1% FA), 10% to 50% gradient in 30 min; detector, UV 254 nm. This resulted in Compound 61 (109 mg, 30%) as a yellow solid.

LC-MS-Compound 61: (ES, m/z): [M-COOH]⁺ 979

H-NMR-Compound 61: (400 MHz, DMSO-d6, δ ppm): 1.09-1.17 (m, 2H), 1.49-1.54 (m, 1H), 1.68-1.71 (m, 2H), 1.94-1.97 (m, 1H), 2.02-2.04 (m, 2H), 2.15-2.17 (m, 2H), 2.36-2.47 (m, 16H), 2.51-2.60 (m, 2H), 2.86-2.97 (m, 7H), 3.28-3.36 (m, 5H), 3.53 (s, 2H), 4.18-4.22 (m, 1H), 4.31-4.35 (m, 1H), 4.91-5.07 (m, 5H), 6.89-6.91 (m, 1H), 7.00-7.06 (m, 3H), 7.32 (s, 1H), 7.38-7.42 (m, 2H), 7.51-7.53 (d, 1H), 7.67 (s, 1H), 7.74-7.76 (d, 1H), 8.19 (s, 1H), 8.20 (s, 1H), 10.95 (s, 1H).

### Example 60. Compound 62

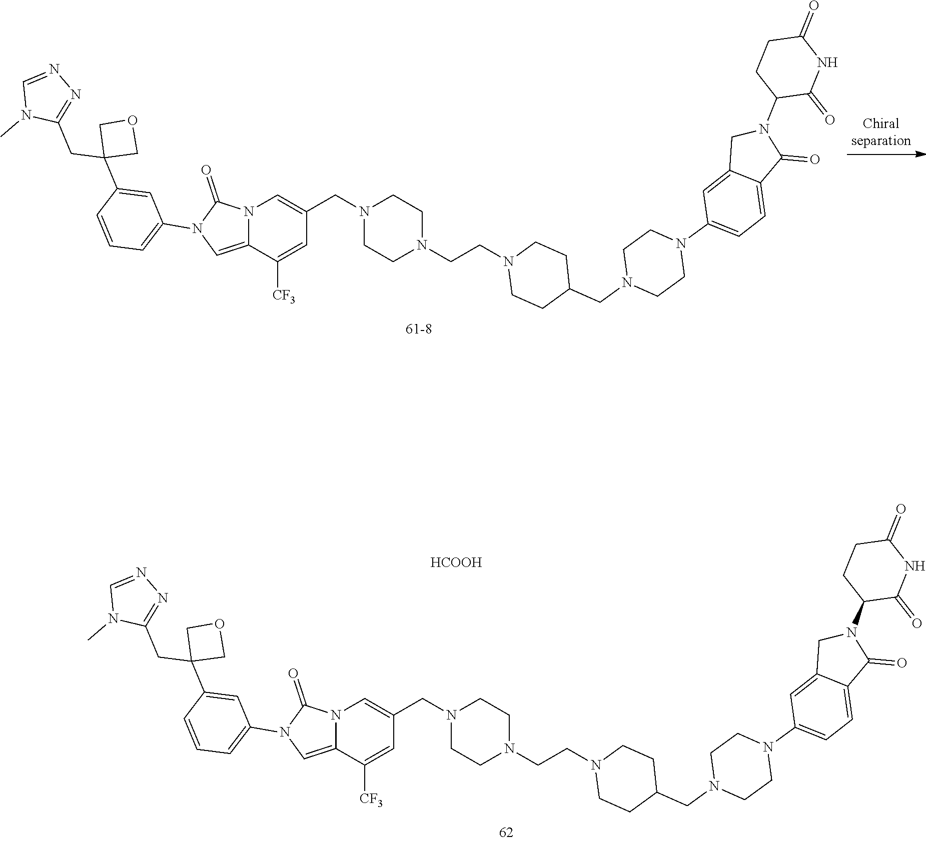 [SMILES: Cn1cnnc1CC1(c2cccc(-n3cc4c(C(F)(F)F)cc(CN5CCN(CCN6CCC(CN7CCN(c8ccc9c(c8)CN(C8CCC(=O)NC8=O)C9=O)CC7)CC6)CC5)cn4c3=O)c2)COC1 ; Cn1cnnc1CC1(c2cccc(-n3cc4c(C(F)(F)F)cc(CN5CCN(CCN6CCC(CN7CCN(c8ccc9c(c8)CN([C@H]8CCC(=O)NC8=O)C9=O)CC7)CC6)CC5)cn4c3=O)c2)COC1 ; "HCOOH"]

### Synthesis of Compound 62.

The Compound 61-6 (330 mg) was purified by Prep-CHIRAL-HPLC with the following conditions Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 60% B to 60% B in 40 min; Wave Length: 220/254 nm; RT1 (min): 16.64; RT2 (min): 20.05; Sample Solvent: DCM:ACN=1:1—HPLC; Injection Volume: 1.5 mL; Number of Runs: 5. The second peak was the product. The residue was purified by reversed-phase flash chromatography with the following conditions: column, C18 silica gel; mobile phase, MeCN in water (0.1% FA), 10% to 50% gradient in 30 min; detector, UV 254 nm. This resulted in Compound 62 (102 mg, 29%) as a yellow solid.

LC-MS-Compound 62: (ES, m/z): [(M−HCOOH)/2+H]⁺ 490

H-NMR-Compound 62: (400 MHz, DMSO-d6, δ ppm): 1.09-1.17 (m, 2H), 1.49-1.54 (m, 1H), 1.68-1.71 (m, 2H), 1.94-1.97 (m, 1H), 2.02-2.04 (m, 2H), 2.15-2.17 (m, 2H), 2.36-2.47 (m, 16H), 2.51-2.60 (m, 2H), 2.86-2.97 (m, 7H), 3.28-3.36 (m, 5H), 3.53 (s, 2H), 4.18-4.22 (m, 1H), 4.31-4.35 (m, 1H), 4.91-5.07 (m, 5H), 6.89-6.91 (m, 1H), 7.00-7.06 (m, 3H), 7.32 (s, 1H), 7.38-7.42 (m, 2H), 7.51-7.53 (d, 1H), 7.67 (s, 1H), 7.74-7.76 (d, 1H), 8.19 (s, 1H), 8.20 (s, 1H), 10.95 (s, 1H).

### Example 61. Compound 63

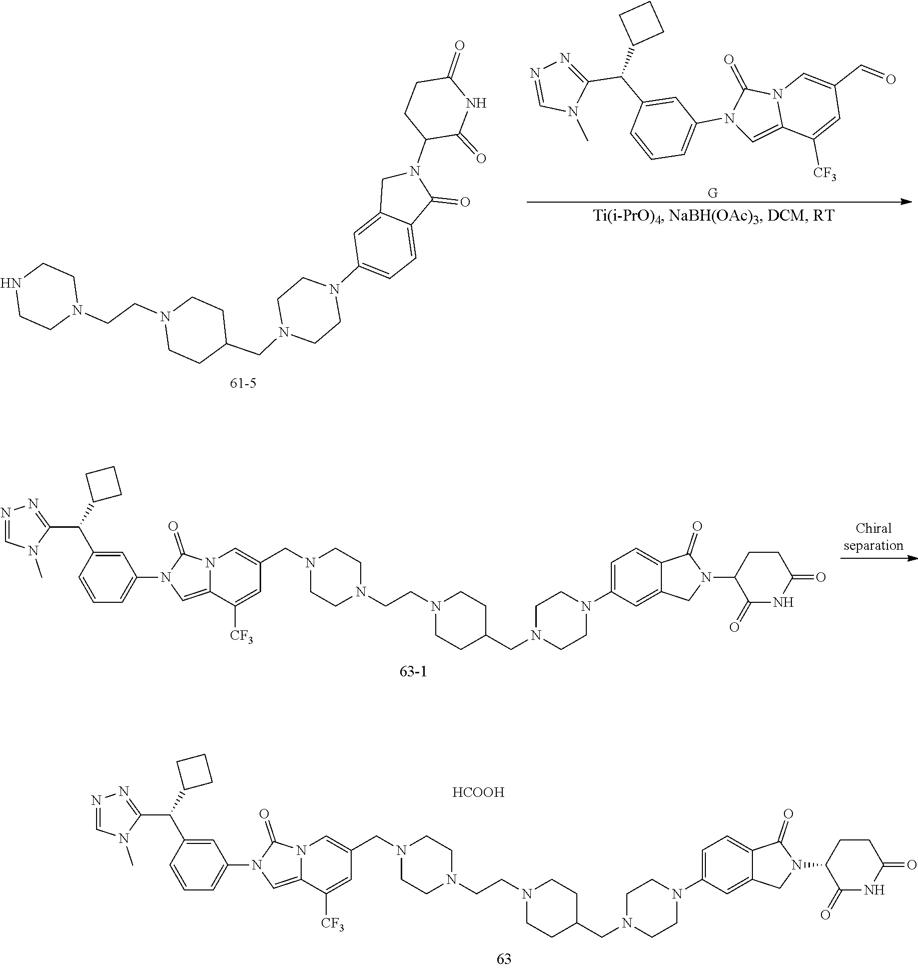

### Synthesis of Compound 63-1.

To a stirred solution of Compound 61-5 (1 g, 1.86 mmol, 1.0 equiv) and Intermediate G (1.3 g, 2.79 mmol, 1.5 equiv) in DCM (10 mL) was added Ti(Oi-Pr)₄ (2.1 g, 7.44 mmol, 4.0 equiv) at room temperature. The resulting mixture was stirred for overnight at room temperature. To the above mixture was added NaBH(OAc)₃ (1.2 g, 5.58 mmol, 3.0 equiv) at room temperature. The resulting mixture was stirred for additional 2 h at room temperature. The reaction was quenched by the addition of 10% AcOH (100 mL) at room temperature. The aqueous layer was extracted with EtOAc (2×100 mL). The aqueous phase was concentrated under reduced pressure. The residue was purified by reversed-phase flash chromatography with the following conditions: column, C18 silica gel; mobile phase, MeCN in water (0.1% FA), 10% to 50% gradient in 30 min; detector, UV 254 nm. This resulted in Compound 63-1 (290 mg, 16%) as a yellow solid.

### Synthesis of Compound 63.

The Compound 63-1 (100 mg) was purified by Prep-CHIRAL-HPLC with the following conditions Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 60% B to 60% B in 10 min; Wave Length: 220/254 nm; RT1 (min): 6.46; RT2 (min): 8.63; Sample Solvent: DCM:ACN=1:1—HPLC; Injection Volume: 1 mL; Number of Runs: 4. The first peak was the product. The residue was purified by reversed-phase flash chromatography with the following conditions: column, C18 silica gel; mobile phase, MeCN in water (0.1% FA), 10% to 50% gradient in 30 min; detector, UV 254 nm. This resulted in Compound 63 (35 mg, 33%) as a yellow solid.

LC-MS-Compound 63: (ES, m/z): [(M−HCOOH)/2+H]⁺ 489

H-NMR-Compound 63: (400 MHz, DMSO-d6 δ ppm): 1.08-1.18 (m, 2H), 1.19-1.25 (m, 2H), 1.41-1.51 (m, 1H), 1.61-1.69 (m, 3H), 1.79-1.81 (m, 4H), 1.92-1.97 (m, 3H), 2.05-2.10 (m, 2H), 2.15-2.17 (m, 2H), 2.34-2.41 (m, 11H), 2.46-2.50 (m, 5H), 2.56-2.60 (m, 2H), 2.86-2.89 (m, 3H), 3.12-3.15 (m, 2H), 3.34-3.36 (m, 2H), 3.39 (s, 3H), 4.18-4.35 (m, 3H), 5.01-5.10 (m, 1H), 6.98 (s, 1H), 7.00-7.06 (m, 2H), 7.18-7.20 (d, 1H), 7.31 (s, 1H), 7.42-7.46 (m, 1H), 7.66-7.68 (m, 2H), 7.70-7.73 (d, 1H), 8.25 (s, 1H), 8.33 (m, 3H), 7.76 (s, 1H), 10.95 (s, 1H).

### Example 62. Compound 64

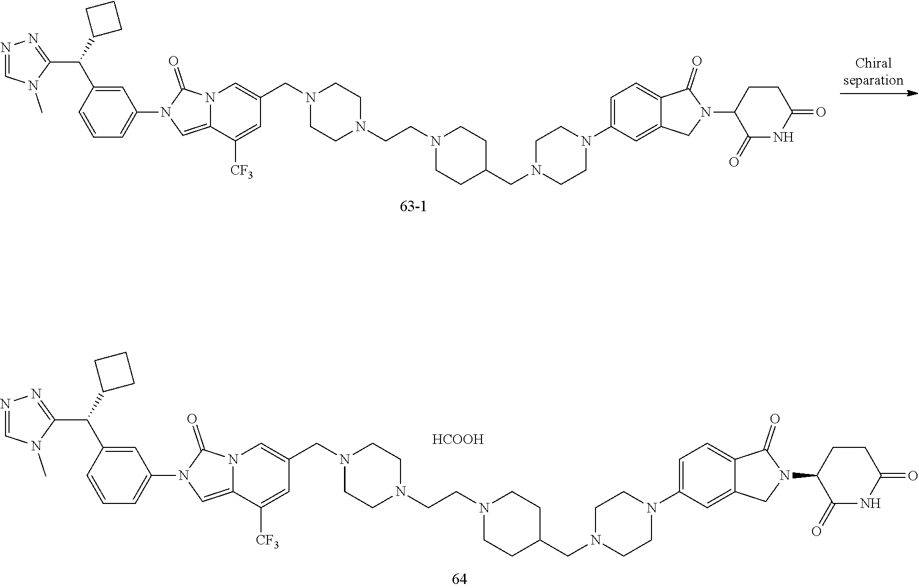 [SMILES: Cn1cnnc1[C@@H](c1cccc(-n2cc3c(C(F)(F)F)cc(CN4CCN(CCN5CCC(CN6CCN(c7ccc8c(c7)CN(C7CCC(=O)NC7=O)C8=O)CC6)CC5)CC4)cn3c2=O)c1)C1CCC1 ; Cn1cnnc1[C@@H](c1cccc(-n2cc3c(C(F)(F)F)cc(CN4CCN(CCN5CCC(CN6CCN(c7ccc8c(c7)CN([C@H]7CCC(=O)NC7=O)C8=O)CC6)CC5)CC4)cn3c2=O)c1)C1CCC1 ; "HCOOH"]

### Synthesis of Compound 64.

The Compound 63-1 (100 mg) was purified by Prep-CHIRAL-HPLC with the following conditions Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 60% B to 60% B in 10 min; Wave Length: 220/254 nm; RT1 (min): 6.46; RT2 (min): 8.63; Sample Solvent: DCM:ACN=1:1—HPLC; Injection Volume: 1 mL; Number of Runs: 4. The second peak was the product. The residue was purified by reversed-phase flash chromatography with the following conditions: column, C18 silica gel; mobile phase, MeCN in water (0.1% FA), 10% to 50% gradient in 30 min; detector, UV 254 nm. This resulted in Compound 64 (32 mg, 30%) as a yellow solid.

LC-MS-Compound 64: (ES, m/z): [M-COOH]⁺ 977

H-NMR-Compound 64: (400 MHz, DMSO-d6, δ ppm): 1.08-1.18 (m, 2H), 1.19-1.25 (m, 2H), 1.41-1.51 (m, 1H), 1.61-1.69 (m, 3H), 1.79-1.81 (m, 4H), 1.92-1.97 (m, 3H), 2.05-2.10 (m, 2H), 2.15-2.17 (m, 2H), 2.34-2.41 (m, 11H), 2.46-2.50 (m, 5H), 2.56-2.60 (m, 2H), 2.86-2.89 (m, 3H), 3.12-3.15 (m, 2H), 3.34-3.36 (m, 2H), 3.39 (s, 3H), 4.18-4.35 (m, 3H), 5.01-5.10 (m, 1H), 6.98 (s, 1H), 7.00-7.06 (m, 2H), 7.18-7.20 (d, 1H), 7.31 (s, 1H), 7.42-7.46 (m, 1H), 7.66-7.68 (m, 2H), 7.70-7.73 (d, 1H), 8.25 (s, 1H), 8.33 (m, 3H), 7.76 (s, 1H), 10.95 (s, 1H).

### Example 63. Compound 65

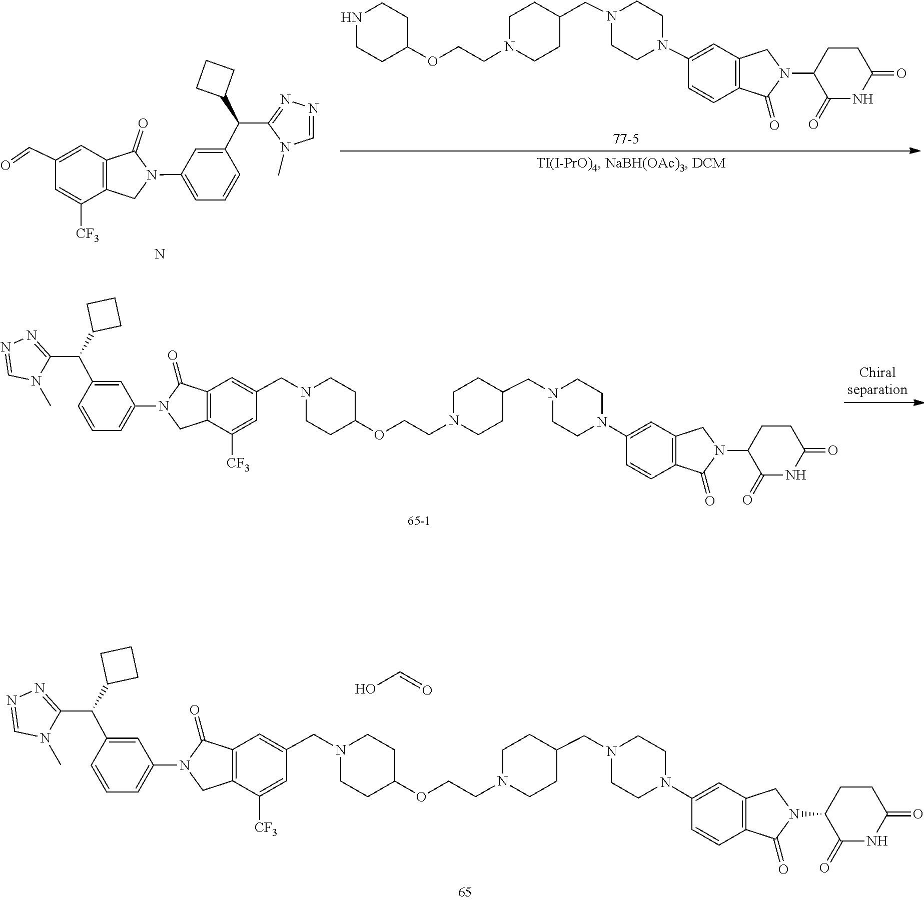

### Synthesis of Compound 65-1.

To a stirred solution of Intermediate N (600 mg, 1.320 mmol, 1 equiv) and Compound 77-5 (729.74 mg, 1.320 mmol, 1 equiv) in DCM (12 mL) was added Ti(Oi-Pr)₄ (1500.98 mg, 5.280 mmol, 4 equiv). The resulting mixture was stirred for overnight at room temperature. To the above mixture was added NaBH(OAc)₃ (839.45 mg, 3.960 mmol, 3 equiv). The resulting mixture was stirred for additional 4 h at room temperature. The reaction was quenched by the addition of Water/HOAc=10/1 (100 mL). The resulting mixture was extracted with CH₂Cl₂/MeOH=10/1 (3×100 mL). The combined organic layers were concentrated under reduced pressure. The residue was purified by reversed-phase flash chromatography with the following conditions: column, C18 silica gel; mobile phase, MeCN in Water (0.1% FA), 10% to 55% gradient in 30 min; detector, UV 254 nm. This resulted in Compound 65-1 (320 mg, 24.45%) as a yellow solid.

### Synthesis of Compound 65.

Compound 65-1 (500 mg) was separated by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 60% B to 60% B in 11.5 min; Wave Length: 220/254 nm; RT1 (min): 6.61; RT2 (min): 8.35; the first peak was product) to afford Compound 65 (224.9 mg, 42.98%) as a yellow solid.

LC-MS-Compound 65: (ES, m/z): [M−HCOOH+H]⁺ 991

H-NMR-Compound 65: (400 MHz, DMSO-d6, δ ppm): 1.17-1.19 (m, 3H), 1.46-1.48 (m, 2H), 1.52-1.61 (m, 1H), 1.71-1.90 (m, 9H), 1.92-1.99 (m, 1H), 2.11-2.18 (m, 5H), 2.23-2.40 (m, 3H), 2.48-2.50 (m, 4H), 2.61-2.68 (m, 5H), 2.82-2.91 (m, 1H), 3.01-3.04 (m, 2H), 3.27-3.33 (m, 6H), 3.43 (s, 3H), 3.55-3.58 (m, 2H), 3.69 (s, 2H), 4.18-4.24 (m, 2H), 4.31-4.35 (d, 1H), 5.03-5.07 (m, 1H), 5.17 (s, 2H), 7.04-7.06 (m, 3H), 7.36-7.40 (m, 1H), 7.51-7.53 (m, 1H), 7.75-7.77 (m, 1H), 7.91 (s, 2H), 7.97 (s, 1H), 8.19 (s, 2H), 8.33 (s, 1H), 10.95 (s, 1H).

### Example 64. Compound 66

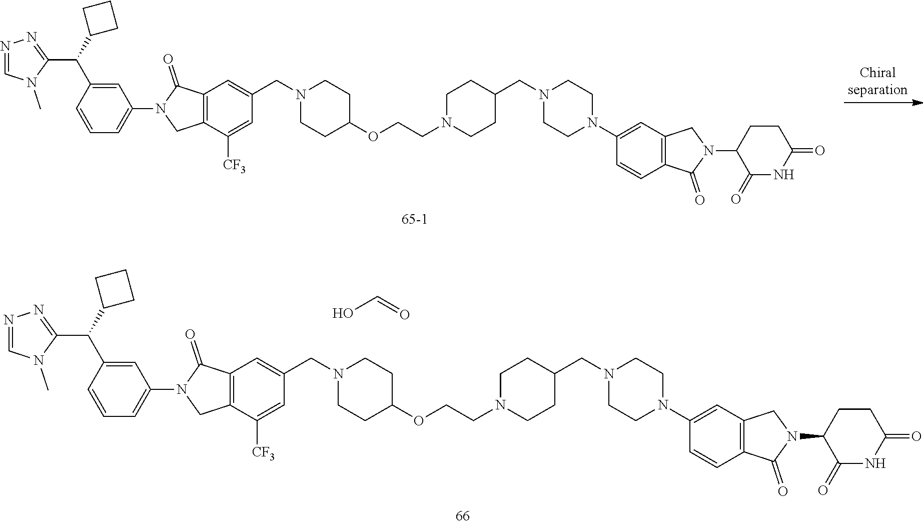 [SMILES: Cn1cnnc1[C@@H](c1cccc(N2Cc3c(cc(CN4CCC(OCCN5CCC(CN6CCN(c7ccc8c(c7)CN(C7CCC(=O)NC7=O)C8=O)CC6)CC5)CC4)cc3C(F)(F)F)C2=O)c1)C1CCC1 ; Cn1cnnc1[C@@H](c1cccc(N2Cc3c(cc(CN4CCC(OCCN5CCC(CN6CCN(c7ccc8c(c7)CN([C@H]7CCC(=O)NC7=O)C8=O)CC6)CC5)CC4)cc3C(F)(F)F)C2=O)c1)C1CCC1 ; O=CO]

### Synthesis of Compound 66.

Compound 65-1 (500 mg) was separated by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 60% B to 60% B in 11.5 min; Wave Length: 220/254 nm; RT1 (min): 6.61; RT2 (min): 8.35; the second peak was product) to afford Compound 66 (224.1 mg, 42.83%) as a yellow solid.

LC-MS-Compound 66: (ES, m/z): [M−HCOOH+H]⁺ 991

H-NMR-Compound 66: (400 MHz, DMSO-d6, δ ppm): 1.10-1.13 (m, 2H), 1.45-1.49 (m, 3H), 1.66-1.80 (m, 9H), 1.94-2.16 (m, 8H), 2.25-2.40 (m, 5H), 2.48-2.50 (m, 2H), 2.61-2.68 (m, 3H), 2.82-2.92 (m, 3H), 3.12-3.32 (m, 6H), 3.43 (s, 3H), 3.51-3.53 (m, 2H), 3.68 (s, 2H), 4.18-4.35 (m, 3H), 5.03-5.07 (m, 1H), 5.17 (s, 2H), 7.04-7.06 (m, 3H), 7.36-7.40 (m, 1H), 7.51-7.53 (m, 1H), 7.75-7.77 (m, 1H), 7.91 (s, 2H), 7.97 (s, 1H), 8.21 (s, 1H), 8.32 (s, 1H), 10.95 (s, 1H).

### Example 65. Compound 67

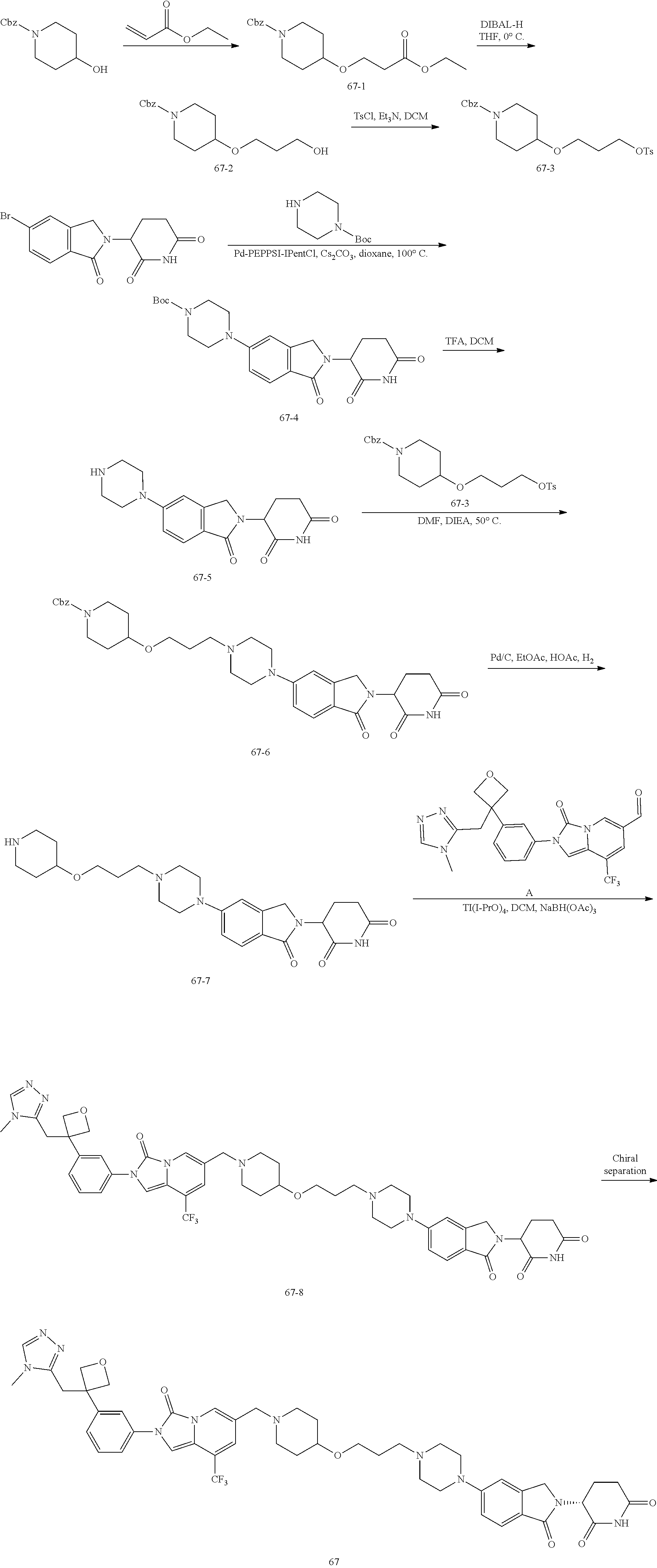

### Synthesis of Compound 67-1.

To a stirred solution of benzyl 4-hydroxypiperidine-1-carboxylate (30 g, 127.50 mmol, 1.0 equiv) and ethyl acrylate (25.5 g, 255.01 mmol, 2.0 equiv) in THF (300 mL) was added KOH (8.6 g, 153.00 mmol, 1.2 equiv) at room temperature. The resulting mixture was stirred for overnight at room temperature. The resulting mixture was diluted with water (300 mL). The mixture was acidified to pH 6 with 1 M HCl (aq.). The resulting mixture was extracted with CH₂Cl₂ (3×300 mL). The combined organic layers were washed with water (3×300 mL), dried over anhydrous Na₂SO₄. After filtration, the filtrate was concentrated under reduced pressure. The residue was purified by silica gel column chromatography, eluted with PE/EA (15:1) to afford Compound 67-1 (15.8 g, 36%) as a colorless oil.

### Synthesis of Compound 67-2.

To a stirred solution of Compound 67-1 (15.6 g, 46.51 mmol, 1.0 equiv) in THF (160 mL) was added DIBAL-H (26.5 g, 186.04 mmol, 4.0 equiv) at 0° C. under nitrogen atmosphere. The resulting mixture was stirred for 4 h at 0° C. under nitrogen atmosphere. The resulting mixture was diluted with water (6.4 mL). The mixture was diluted with with 15% NaOH (6.4 ml). The resulting mixture was diluted with water (16 mL). The mixture was allowed to room temperature. The resulting mixture was stirred for 15 min at room temperature. To the above mixture was added MgSO₄ (10 g) at room temperature. The resulting mixture was stirred for 15 min at room temperature. The resulting mixture was filtered; the filter cake was washed with EtOAc (3×50 mL). The filtrate was concentrated under reduced pressure. The residue was purified by silica gel column chromatography, eluted with PE/EA (10:1) to afford Compound 67-2 (4.6 g, 33%) as a colorless oil.

### Synthesis of Compound 67-3.

To a stirred solution of Compound 67-2 (2 g, 6.81 mmol, 1.0 equiv) and Et₃N (1.4 g, 13.63 mmol, 2.0 equiv) in DCM (20 mL) was added TsCl (2.6 g, 13.64 mmol, 2.0 equiv) at room temperature. The resulting mixture was stirred for overnight at room temperature. The reaction was quenched by the addition of sat. NH₄Cl (aq.) (50 mL) at room temperature. The resulting mixture was extracted with CH₂Cl₂ (3×100 mL). The combined organic layers were washed with water (3×100 mL), dried over anhydrous Na₂SO₄. After filtration, the filtrate was concentrated under reduced pressure. The residue was purified by silica gel column chromatography, eluted with PE/EA (1:1) to afford Compound 67-3 (2.8 g, 92%) as an off-white solid.

### Synthesis of Compound 67-4.

To a stirred solution of 3-(5-bromo-1-oxo-3H-isoindol-2-yl) piperidine-2,6-dione (1 g, 3.09 mmol, 1.0 equiv) and tert-butyl piperazine-1-carboxylate (0.6 g, 3.09 mmol, 1.0 equiv) in 1,4-dioxane (10 mL) were added Cs₂CO₃ (3 g, 9.28 mmol, 3.0 equiv) and Pd PEPPSI IPentCl (0.3 g, 0.31 mmol, 0.1 equiv) at room temperature under nitrogen atmosphere. The resulting mixture was stirred for overnight at 100° C. under nitrogen atmosphere. The reaction was quenched by the addition of 10% AcOH (aq.) (100 mL) at room temperature. The resulting mixture was concentrated under reduced pressure. The residue was purified by silica gel column chromatography, eluted with CH₂Cl₂/MeOH (10:1) to afford Compound 67-4 (630 mg, 48%) as an off-white solid.

### Synthesis of Compound 67-5.

A solution of Compound 67-4 (630 mg, 1.47 mmol, 1.0 equiv) and TFA (2 mL) in DCM (6 mL) was stirred for overnight at room temperature. The mixture was neutralized to pH 7 with NH₃ in MeOH. The resulting mixture was concentrated under reduced pressure. The residue was purified by Prep-TLC (CH₂Cl₂/MeOH=10:1) to afford Compound 67-5 (450 mg, 93%) as an off-white solid.

### Synthesis of Compound 67-6.

To a stirred solution of Compound 67-5 (3.2 g, 9.74 mmol, 1.0 equiv) and benzyl Compound 67-3 (6.5 g, 14.61 mmol, 1.5 equiv) in DMF (32 mL) was added DIEA (2.5 g, 19.49 mmol, 2.0 equiv) at room temperature. The resulting mixture was stirred for overnight at 50° C. The reaction was quenched by the addition of sat. NH₄Cl (aq.) (100 mL) at room temperature. The resulting mixture was extracted with EtOAc (3×300 mL). The combined organic layers were washed with water (3×150 mL), dried over anhydrous Na₂SO₄. After filtration, the filtrate was concentrated under reduced pressure. The residue was purified by silica gel column chromatography, eluted with PE/EA (1:1) to afford Compound 67-6 (3.9 g, 66%) as an off-white solid.

### Synthesis of Compound 67-7.

To a solution of Compound 67 (3.6 g, 5.96 mmol, 1.0 equiv) in 90 mL EtOAc and 18 mL AcOH was added Pd/C (10%, 1.08 g) under nitrogen atmosphere in a 25 mL round-bottom flask. The mixture was hydrogenated at room temperature for 2 h under hydrogen atmosphere using a hydrogen balloon, filtered through a Celite pad and concentrated under reduced pressure. This resulted in Compound 67-7 (2.7 g, 96%) as an off-white solid.

### Synthesis of Compound 67-8.

To a stirred solution of Compound 67-7 (1.3 g, 2.78 mmol, 1.0 equiv) and Intermediate A (1.9 g, 4.15 mmol, 1.5 equiv) in DCM (13 mL) was added Ti(Oi-Pr)₄ (3.1 g, 11.07 mmol, 4.0 equiv) at room temperature. The resulting mixture was stirred for overnight at room temperature. To the above mixture was added NaBH(OAc)₃ (1.7 g, 8.30 mmol, 3.0 equiv) at room temperature. The resulting mixture was stirred for additional 2 h at room temperature. The reaction was quenched by the addition of 10% AcOH (mL) at room temperature. The aqueous layer was extracted with EtOAc (3×100 mL). The aqueous phase was concentrated under reduced pressure. The residue was purified by reversed-phase flash chromatography with the following conditions: column, C18 silica gel; mobile phase, MeCN in water (0.1% FA), 10% to 50% gradient in 30 min; detector, UV 254 nm. This resulted in Compound 67-8 (220 mg, 9%) as a yellow solid.

### Synthesis of Compound 67.

The Compound 67-8 (220 mg) was purified by Prep-CHIRAL-HPLC with the following conditions Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 60% B to 60% B in 13 min; Wave Length: 220/254 nm; RT1 (min): 4.92; RT2 (min): 7.83; Sample Solvent: DCM:ACN=1:1—HPLC; Injection Volume: 0.8 mL; Number of Runs: 6. The first peak was the product. This resulted in Compound 67 (99 mg, 44%) as a yellow solid.

LC-MS-Compound 67: (ES, m/z): [M+H]⁺ 911

H-NMR-Compound 67: (400 MHz, DMSO-d6, δ ppm): 1.44-1.46 (m, 2H), 1.66-1.69 (m, 2H), 1.81-1.83 (m, 2H), 1.91-1.98 (m, 1H), 2.13-2.17 (m, 2H), 2.34-2.40 (m, 1H), 2.57-2.61 (m, 1H), 2.68-2.70 (m, 2H), 2.91-2.95 (m, 1H), 2.97 (s, 3H), 3.29-3.34 (m, 8H), 3.43-3.46 (m, 2H), 3.54 (s, 2H), 4.18-4.22 (d, 1H), 4.31-4.35 (d, 1H), 4.91-4.96 (m, 4H), 5.03-5.08 (m, 1H), 6.88-6.90 (d, 1H), 7.02-7.07 (m, 3H), 7.33 (s, 1H), 7.38-7.42 (m, 2H), 7.51-7.53 (d, 1H), 7.68 (s, 1H), 7.75-7.77 (d, 1H), 8.21 (s, 1H), 10.95 (s, 1H).

### Example 66. Compound 68

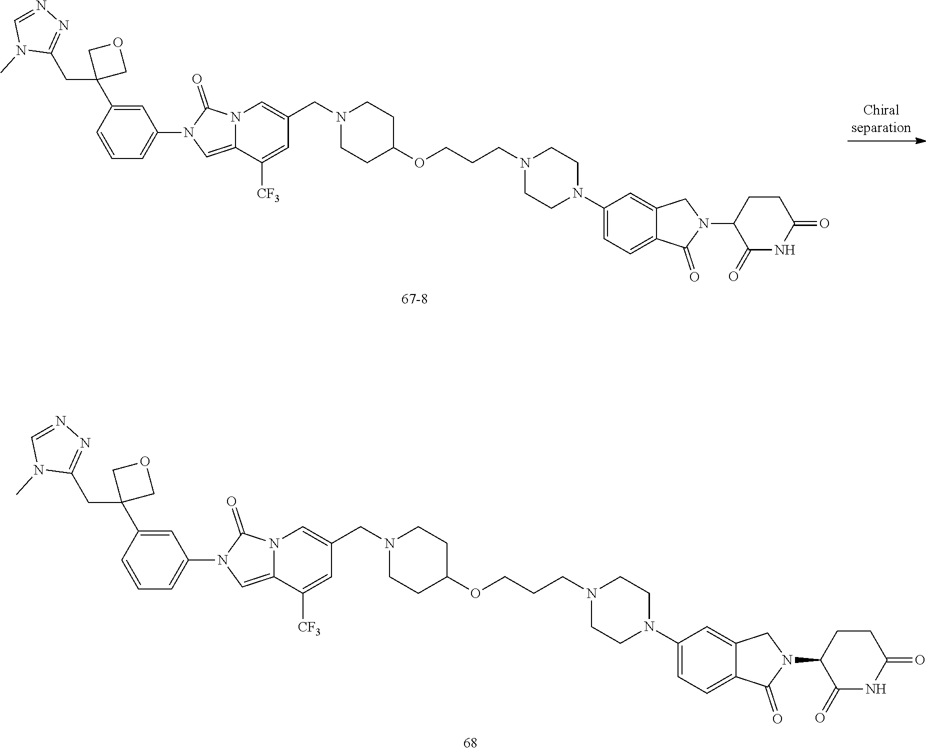 [SMILES: Cn1cnnc1CC1(c2cccc(-n3cc4c(C(F)(F)F)cc(CN5CCC(OCCCN6CCN(c7ccc8c(c7)CN(C7CCC(=O)NC7=O)C8=O)CC6)CC5)cn4c3=O)c2)COC1 ; Cn1cnnc1CC1(c2cccc(-n3cc4c(C(F)(F)F)cc(CN5CCC(OCCCN6CCN(c7ccc8c(c7)CN([C@H]7CCC(=O)NC7=O)C8=O)CC6)CC5)cn4c3=O)c2)COC1]

### Synthesis of Compound 68.

The Compound 67-8 (220 mg) was purified by Prep-CHIRAL-HPLC with the following conditions Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 60% B to 60% B in 13 min; Wave Length: 220/254 nm; RT1 (min): 4.92; RT2 (min): 7.83; Sample Solvent: DCM:ACN=1:1—HPLC; Injection Volume: 0.8 mL; Number of Runs: 6. The second peak was the product. This resulted in Compound 68 (95 mg, 43%) as a yellow solid.

LC-MS-Compound 68: (ES, m/z): [M+H]⁺ 911

H-NMR-Compound 68: (400 MHz, DMSO-d6, δ ppm): 1.44-1.46 (m, 2H), 1.66-1.69 (m, 2H), 1.81-1.83 (m, 2H), 1.91-1.98 (m, 1H), 2.13-2.17 (m, 2H), 2.34-2.40 (m, 1H), 2.57-2.61 (m, 1H), 2.68-2.70 (m, 2H), 2.91-2.95 (m, 1H), 2.97 (s, 3H), 3.29-3.34 (m, 8H), 3.43-3.46 (m, 2H), 3.54 (s, 2H), 4.18-4.22 (d, 1H), 4.31-4.35 (d, 1H), 4.91-4.96 (m, 4H), 5.03-5.08 (m, 1H), 6.88-6.90 (d, 1H), 7.02-7.07 (m, 3H), 7.33 (s, 1H), 7.38-7.42 (m, 2H), 7.51-7.53 (d, 1H), 7.68 (s, 1H), 7.75-7.77 (d, 1H), 8.21 (s, 1H), 10.95 (s, 1H).

### Example 67. Compound 69

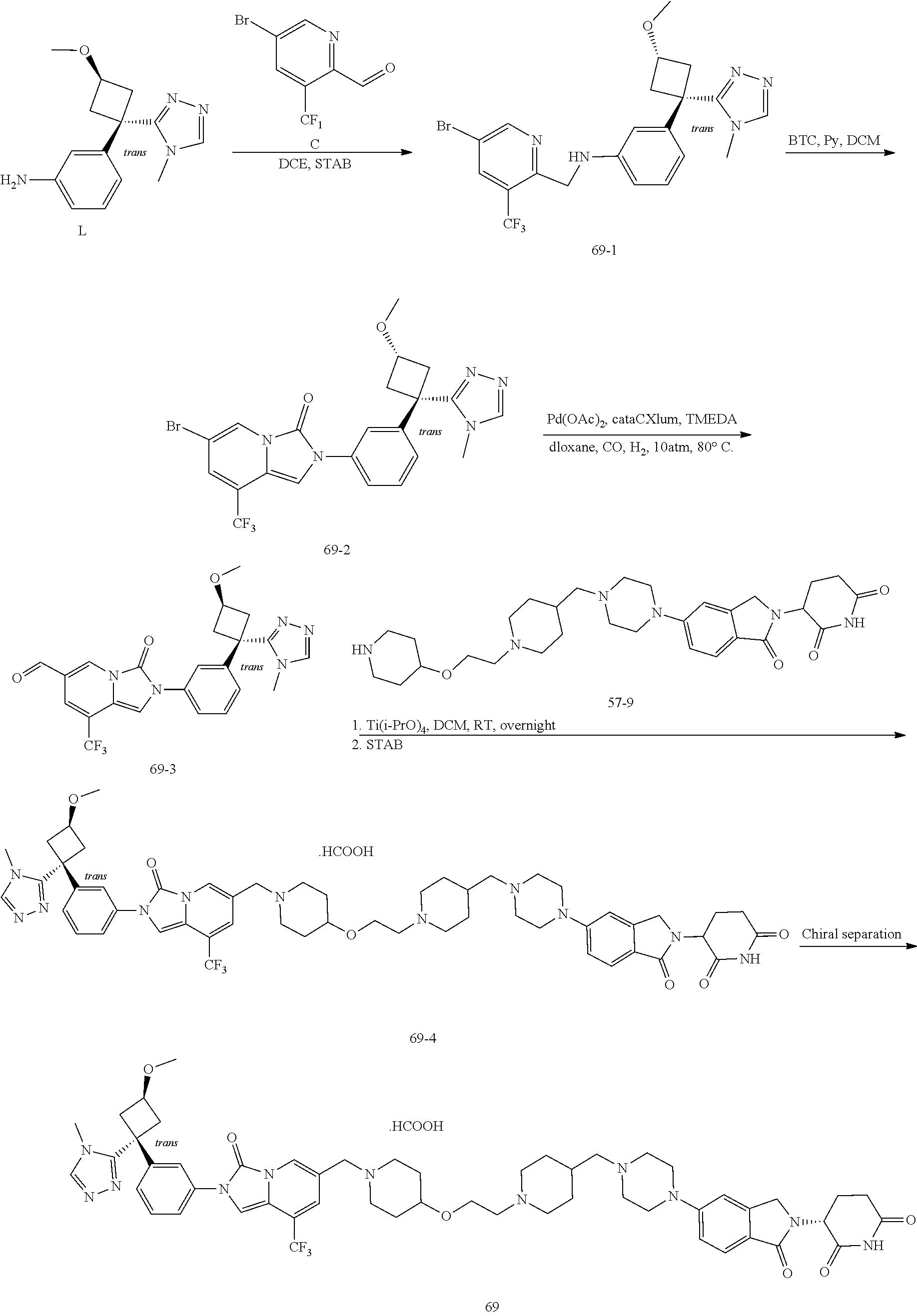

### Synthesis of Compound 69-1.

To a stirred solution of Intermediate L (1.6 g, 6.194 mmol, 1 equiv) in DCE (30 mL) were Intermediate C (2.05 g, 8.052 mmol, 1.3 equiv) at room temperature. The resulting mixture was stirred overnight at room temperature. To the above mixture was added STAB (2.63 g, 12.388 mmol, 2 equiv) at room temperature. The resulting mixture was stirred for additional 4 h at room temperature. The reaction was quenched with saturated NaHCO₃ (aq.) (150 mL) at room temperature. The aqueous layer was extracted with DCM (2×100 mL). The resulting mixture was concentrated under vacuum. The product was precipitated by the addition of MTBE (60 mL). This resulted in Compound 69-1 (2 g, 61.81%) as an off-white solid.

### Synthesis of Compound 69-2.

To a stirred solution of Compound 69-1 (0.2 g, 4.030 mmol, 1 equiv) and pyridine (3.19 g, 40.300 mmol, 10 equiv) in DCM (50 mL) was added triphosgene (0.48 g, 1.612 mmol, 0.4 equiv) at 0° C. The resulting mixture was stirred for 10 min at room temperature. The reaction was quenched with saturated NaHCO₃ (aq.) (100 mL) at room temperature. The resulting mixture was extracted with DCM (2×100 mL) and dried over anhydrous Na₂SO₄. After filtration, the filtrate was concentrated under reduced pressure. The product was precipitated by the addition of MTBE (50 mL). This resulted in Compound 69-2 (1.6 g, 72.22%) as a yellow solid.

### Synthesis of Compound 69-3.

To a solution of Compound 69-2 (1.6 g, 3.063 mmol, 1 equiv) in dioxane (30 mL) was added Pd(OAc)₂ (0.07 g, 0.306 mmol, 0.1 equiv), bis(adamantan-1-yl)(butyl)phosphane (0.22 g, 0.613 mmol, 0.2 equiv) and TMEDA (0.71 g, 6.126 mmol, 2 equiv) in a pressure tank. The mixture was purged with nitrogen for 2 min and then was pressurized to 10 atm with H₂/CO=1:1 at 80° C. for overnight. The reaction mixture was cooled to room temperature and concentrated under vacuum. The residue was purified by silica gel column chromatography, eluted with DCM/MeOH (20:1) to afford Compound 69-3 (1 g, 64.40%) as a yellow solid.

### Synthesis of Compound 69-4.

To a stirred solution of Compound 69-3 (600 mg, 1.273 mmol, 1 equiv) and Compound 57-9 (597.93 mg, 1.082 mmol, 0.85 equiv) in DCM (25 mL) was added Ti(Oi-Pr)₄ (1085.17 mg, 3.819 mmol, 3 equiv) at room temperature. The resulting mixture was stirred overnight at room temperature. To the above mixture was added STAB (809.21 mg, 3.819 mmol, 3 equiv) at room temperature. The resulting mixture was stirred for additional 6 h at room temperature. The reaction was quenched with 10% HOAc (aq.) (100 mL) at room temperature. The aqueous layer was extracted with EtOAc (50 mL). The aqueous layer was filtered, the filter cake was washed with water (10 mL). The filtrate was concentrated under reduced pressure. The residue was purified by reverse flash chromatography with the following conditions: column, C18 silica gel; mobile phase, MeCN in water (0.1% FA), 10% to 50% gradient in 20 min; detector, UV 254 nm. This resulted in Compound 69-4 (450 mg, 34.02%) as a yellow solid.

### Synthesis of Compound 69.

The Compound 69-4 (450 mg) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 60% B to 60% B in 11 min; Wave Length: 220/254 nm; RT1 (min): 4.83; RT2 (min): 6.66; the first peak is product) to afford the crude product. The crude product was purified by reverse flash chromatography with the following conditions: column, C18 silica gel; mobile phase, MeCN in water (0.1% FA), 10% to 50% gradient in 10 min; detector, UV 254 nm. This resulted in Compound 69 (165.7 mg, 34.62%) as a yellow solid.

LC-MS-Compound 69: (ES, m/z): [M−HCOOH+H]⁺ 1008

H-NMR-Compound 69: (400 MHz, DMSO-d6, δ ppm): 1.12-1.24 (m, 2H), 1.41-1.49 (m, 2H), 1.51-1.65 (m, 1H), 1.70-1.79 (m, 2H), 1.81-1.89 (m, 2H), 1.91-2.02 (m, 1H), 2.11-2.25 (m, 6H), 2.31-2.45 (m, 2H), 2.45-2.50 (m, 4H), 2.51-2.61 (m, 1H), 2.61-2.69 (m, 3H), 2.69-2.73 (m, 2H), 2.85-3.05 (m, 3H), 3.17 (s, 3H), 3.24-3.45 (m, 12H), 3.53-3.56 (m, 2H), 3.81-3.92 (m, 1H), 4.18-4.22 (m, 1H), 4.30-4.35 (m, 1H), 5.03-5.13 (m, 1H), 7.01-7.12 (m, 4H), 7.40-7.53 (m, 3H), 7.67-7.70 (m, 3H), 8.21 (s, 3H), 8.39 (s, 1H), 10.95 (s, 1H).

### Example 68. Compound 70

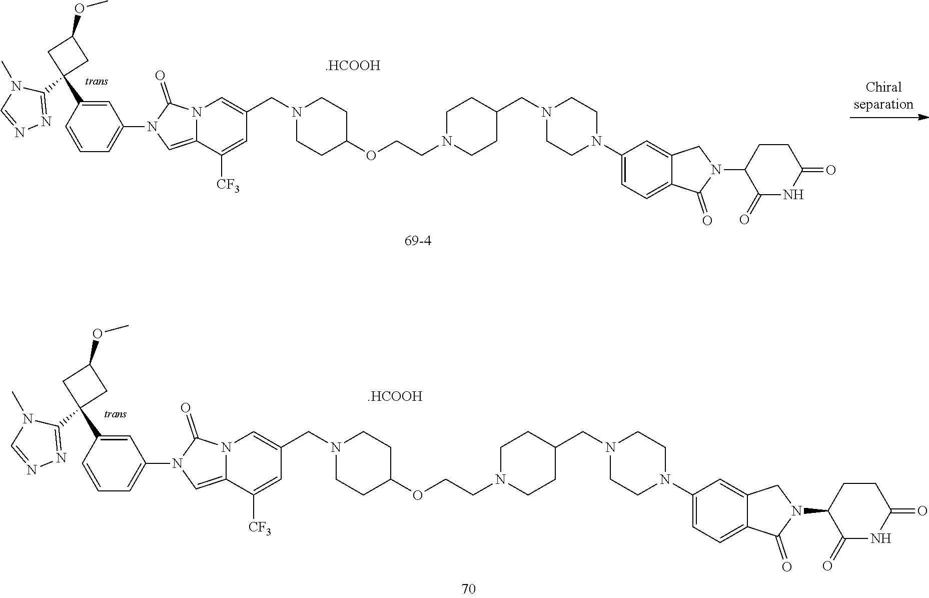 [SMILES: CO[C@H]1C[C@](c2cccc(-n3cc4c(C(F)(F)F)cc(CN5CCC(OCCN6CCC(CN7CCN(c8ccc9c(c8)CN(C8CCC(=O)NC8=O)C9=O)CC7)CC6)CC5)cn4c3=O)c2)(c2nncn2C)C1 ; ".HCOOH" ; CO[C@H]1C[C@](c2cccc(-n3cc4c(C(F)(F)F)cc(CN5CCC(OCCN6CCC(CN7CCN(c8ccc9c(c8)CN([C@H]8CCC(=O)NC8=O)C9=O)CC7)CC6)CC5)cn4c3=O)c2)(c2nncn2C)C1 ; ".HCOOH"]

### Synthesis of Compound 70.

The Compound 69-4 (450 mg) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 60% B to 60% B in 11 min; Wave Length: 220/254 nm; RT1 (min): 4.83; RT2 (min): 6.66; the second peak is product) to afford Compound 70 (201.8 mg, 42.50%) as a yellow solid.

LC-MS-Compound 70: (ES, m/z): [M−HCOOH+H]⁺ 1008

H-NMR-Compound 70: (400 MHz, DMSO-d6, δ ppm): 1.12-1.24 (m, 2H), 1.41-1.49 (m, 2H), 1.51-1.65 (m, 1H), 1.70-1.79 (m, 2H), 1.81-1.89 (m, 2H), 1.91-2.02 (m, 1H), 2.11-2.25 (m, 6H), 2.31-2.45 (m, 2H), 2.45-2.50 (m, 4H), 2.51-2.61 (m, 1H), 2.61-2.69 (m, 3H), 2.69-2.73 (m, 2H), 2.85-3.05 (m, 3H), 3.17 (s, 3H), 3.24-3.45 (m, 12H), 3.53-3.56 (m, 2H), 3.81-3.92 (m, 1H), 4.18-4.22 (m, 1H), 4.30-4.35 (m, 1H), 5.03-5.13 (m, 1H), 7.00 (s, 1H), 7.01-7.12 (m, 3H), 7.40 (s, 1H), 7.46-7.53 (m, 2H), 7.66-7.70 (m, 3H), 8.18 (s, 2H), 8.39 (s, 1H), 10.95 (s, 1H).

### Example 69. Compound 71

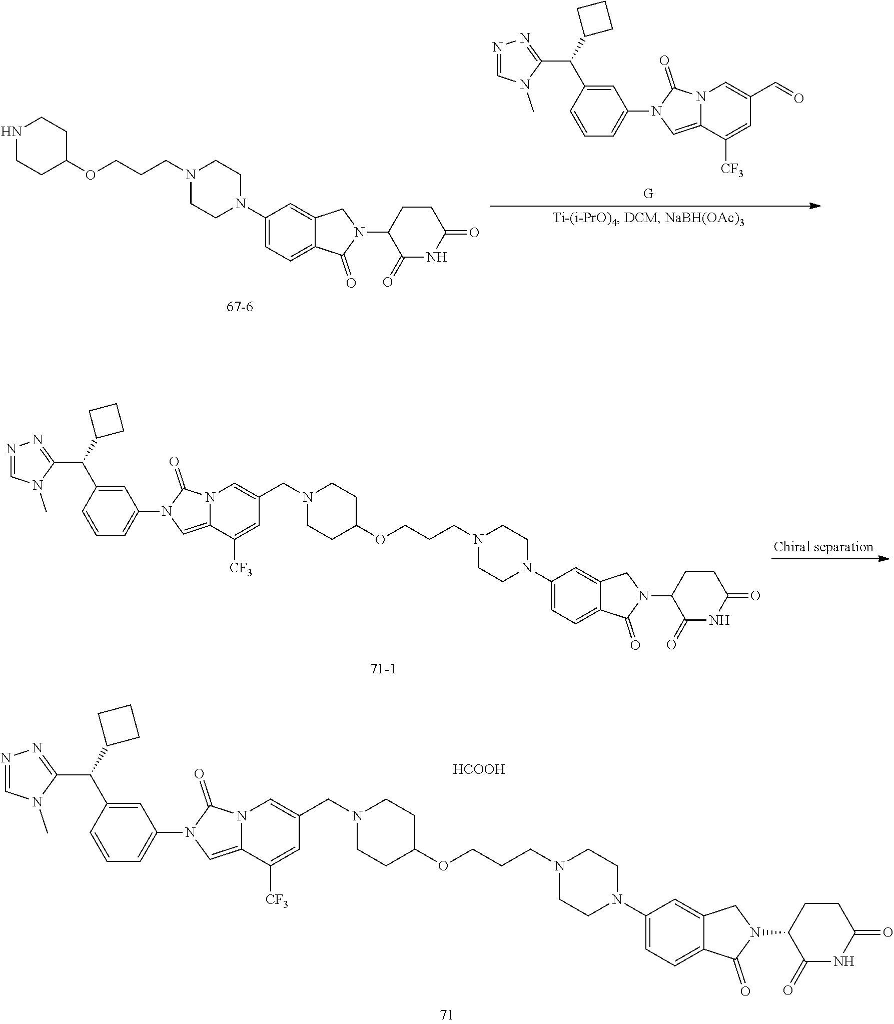 [SMILES: O=C1CCC(N2Cc3cc(N4CCN(CCCOC5CCNCC5)CC4)ccc3C2=O)C(=O)N1 ; Cn1cnnc1[C@@H](c1cccc(-n2cc3c(C(F)(F)F)cc(C=O)cn3c2=O)c1)C1CCC1 ; Cn1cnnc1[C@@H](c1cccc(-n2cc3c(C(F)(F)F)cc(CN4CCC(OCCCN5CCN(c6ccc7c(c6)CN(C6CCC(=O)NC6=O)C7=O)CC5)CC4)cn3c2=O)c1)C1CCC1 ; Cn1cnnc1[C@@H](c1cccc(-n2cc3c(C(F)(F)F)cc(CN4CCC(OCCCN5CCN(c6ccc7c(c6)CN([C@@H]6CCC(=O)NC6=O)C7=O)CC5)CC4)cn3c2=O)c1)C1CCC1 ; "HCOOH"]

### Synthesis of Compound 71-1.

To a stirred solution of Compound 67-6 (1 g, 2.13 mmol, 1.0 equiv) and Intermediate G (1.5 g, 3.19 mmol, 1.5 equiv) in DCM (10 mL) was added Ti(Oi-Pr)₄ (2.4 g, 8.52 nmol, 4.0 equiv) at room temperature. The resulting mixture was stirred for overnight at room temperature. To the above mixture was added NaBH(OAc)₃ (1.3 g, 6.39 mmol, 3.0 equiv) at room temperature. The resulting mixture was stirred for additional 2 h at room temperature. The reaction was quenched by the addition of 10% AcOH (100 mL) at room temperature. The aqueous layer was extracted with EtOAc (2×60 mL). The aqueous phase was concentrated under reduced pressure. The residue was purified by reversed-phase flash chromatography with the following conditions: column, C18 silica gel; mobile phase, MeCN in water (0.1% FA), 10% to 50% gradient in 30 min; detector, UV 254 nm. This resulted in Compound 71-1 (450 mg, 23%) as a yellow solid.

### Synthesis of Compound 71.

The Compound 71-1 (450 mg) was purified by Prep-CHIRAL-HPLC with the following conditions Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 60% B to 60% B in 13 min; Wave Length: 220/254 nm; RT1 (min): 4.92; RT2 (min): 7.83; Sample Solvent: DCM:ACN=1:1—HPLC; Injection Volume: 0.8 mL; Number of Runs: 6. The first peak was the product. The residue was purified by reversed-phase flash chromatography with the following conditions: column, C18 silica gel; mobile phase, MeCN in water (0.1% FA), 10% to 50% gradient in 30 min; detector, UV 254 nm. This resulted in Compound 71 (86 mg, 18%) as a yellow solid.

LC-MS-Compound 71: (ES, m/z): [M-COOH]⁺ 909

H-NMR-Compound 71: (400 MHz, DMSO-d6, δ ppm): 1.45-1.47 (m, 2H), 1.69-1.75 (m, 3H), 1.80-1.81 (m, 6H), 1.96-1.98 (m, 1H), 2.09-2.10 (m, 1H), 2.19 (s, 2H), 2.35-2.42 (m, 3H), 2.51-2.69 (m, 4H), 2.85-2.91 (m, 1H), 3.22-3.44 (m, 16H), 4.18-4.35 (m, 3H), 5.03-5.08 (m, 1H), 7.03-7.07 (m, 3H), 7.19-7.21 (d, 1H), 7.33 (s, 1H), 7.43-7.53 (m, 2H), 7.69-7.74 (m, 3H), 8.16 (s, 1H), 8.34 (s, 1H), 10.95 (s, 1H).

### Example 70. Compound 72

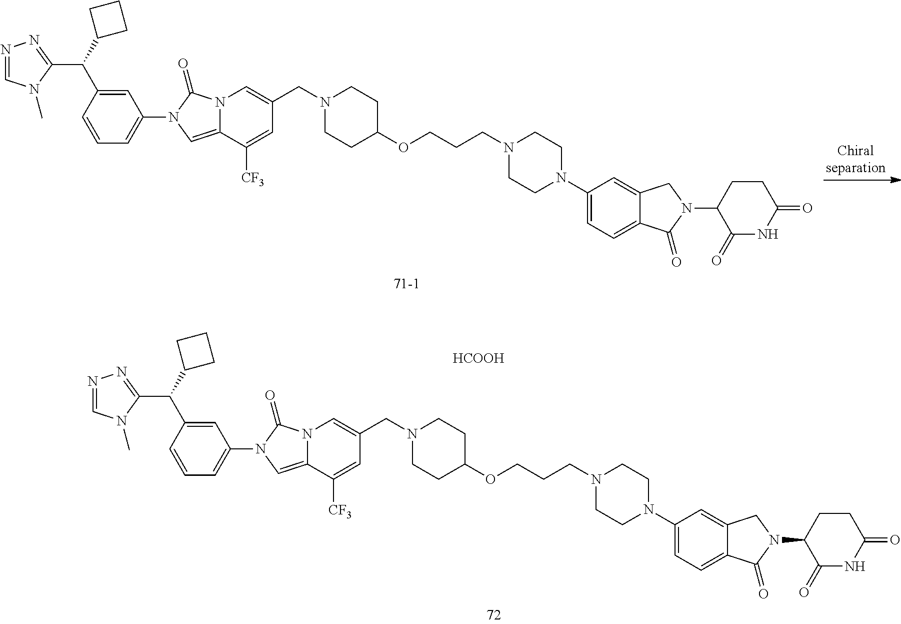 [SMILES: Cn1cnnc1[C@@H](c1cccc(-n2cc3c(C(F)(F)F)cc(CN4CCC(OCCCN5CCN(c6ccc7c(c6)CN(C6CCC(=O)NC6=O)C7=O)CC5)CC4)cn3c2=O)c1)C1CCC1 ; Cn1cnnc1[C@@H](c1cccc(-n2cc3c(C(F)(F)F)cc(CN4CCC(OCCCN5CCN(c6ccc7c(c6)CN([C@H]6CCC(=O)NC6=O)C7=O)CC5)CC4)cn3c2=O)c1)C1CCC1 ; "HCOOH"]

### Synthesis of Compound 72.

The Compound 71-1 (450 mg) was purified by Prep-CHIRAL-HPLC with the following conditions Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 60% B to 60% B in 13 min; Wave Length: 220/254 nm; RT1 (min): 4.92; RT2 (min): 7.83; Sample Solvent: DCM:ACN=1:1—HPLC; Injection Volume: 0.8 mL; Number of Runs: 6. The second peak was the product. The residue was purified by reversed-phase flash chromatography with the following conditions: column, C18 silica gel; mobile phase, MeCN in water (0.1% FA), 10% to 50% gradient in 30 min; detector, UV 254 nm. This resulted in Compound 72 (140 mg, 29%)) as a yellow solid.

LC-MS-Compound 72: (ES, m/z): [M-COOH]⁺ 909

H-NMR-Compound 72: (400 MHz, DMSO-d6, δ ppm): 1.45-1.47 (m, 2H), 1.69-1.75 (m, 3H), 1.80-1.81 (m, 6H), 1.96-1.98 (m, 1H), 2.09-2.10 (m, 1H), 2.19 (s, 2H), 2.35-2.42 (m, 3H), 2.51-2.69 (m, 4H), 2.85-2.91 (m, 1H), 3.22-3.44 (m, 16H), 4.18-4.35 (m, 3H), 5.03-5.08 (m, 1H), 7.03-7.07 (m, 3H), 7.19-7.21 (d, 1H), 7.33 (s, 1H), 7.43-7.53 (m, 2H), 7.69-7.74 (m, 3H), 8.16 (s, 1H), 8.34 (s, 1H), 10.95 (s, 1H).

### Example 71. Compound 73

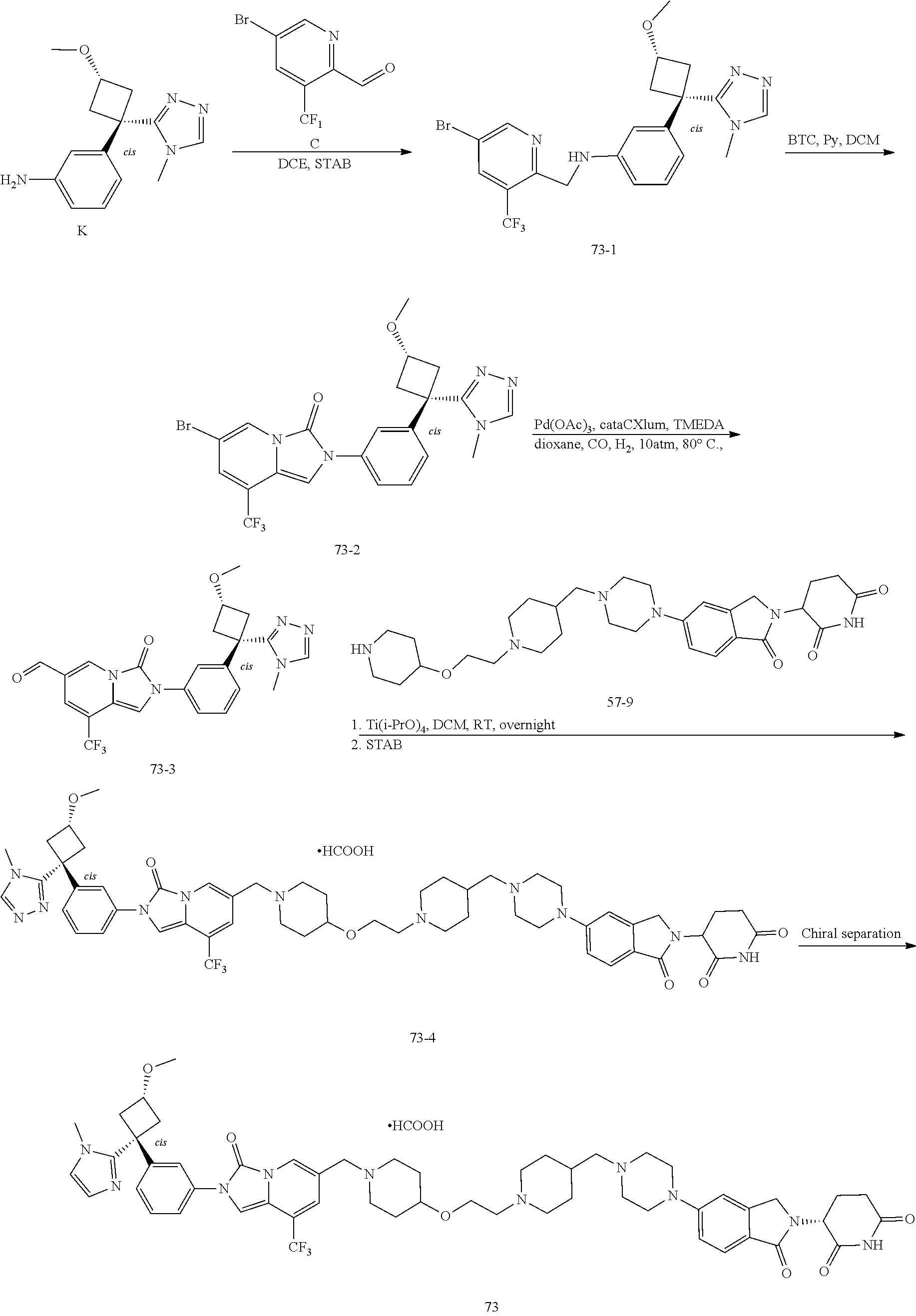

### Synthesis of Compound 73-1.

To a stirred solution of Intermediate K (1.1 g, 4.258 mmol, 1 equiv) in DCE (20 mL) was added Intermediate C (1.41 g, 5.535 mmol, 1.3 equiv) at room temperature. The resulting mixture was stirred overnight at room temperature. To the above mixture was added STAB (1.80 g, 8.516 mmol, 2 equiv) at room temperature. The resulting mixture was stirred for additional 4 h at room temperature. The reaction was quenched with saturated NaHCO₃ (aq.) (150 mL) at room temperature. The aqueous layer was extracted with DCM (2×100 mL). The resulting mixture was concentrated under vacuum. The product was precipitated by the addition of MTBE (60 mL). This resulted in Compound 73-1 (2.2 g, 96.81%) as an off-white solid.

### Synthesis of Compound 73-2.

To a stirred solution of Compound 73-1 (2.1 g, 4.231 mmol, 1 equiv) and pyridine (3.35 g, 42.310 mmol, 10 equiv) in DCM (50 mL) was added triphosgene (0.50 g, 1.692 mmol, 0.4 equiv) at 0° C. The resulting mixture was stirred for 10 min at room temperature. The reaction was quenched with saturated NaHCO₃ (aq.) (100 mL) at room temperature. The resulting mixture was extracted with DCM (2×100 mL) and dried over anhydrous Na₂SO₄. After filtration, the filtrate was concentrated under reduced pressure. The product was precipitated by the addition of MTBE (60 mL). This resulted in Compound 73-1 (2 g, 85.97%) as an orange solid.

### Synthesis of Compound 73-3.

To a solution of Compound 73-2 (2 g, 3.829 mmol, 1 equiv) in dioxane (30 mL) was added Pd(OAc)₂ (0.09 g, 0.383 mmol, 0.1 equiv), bis(adamantan-1-yl)(butyl)phosphane (0.27 g, 0.766 mmol, 0.2 equiv) and TMEDA (0.89 g, 7.658 mmol, 2 equiv) in a pressure tank. The mixture was purged with nitrogen for 2 min and then was pressurized to 10 atm with H₂/CO=1:1 at 80° C. for overnight. The reaction mixture was cooled to room temperature and concentrated under vacuum. The residue was purified by silica gel column chromatography, eluted with DCM/MeOH (20:1) to afford Compound 73-3 (1.2 g, 60.49%) as a yellow solid.

### Synthesis of Compound 73-4.

To a stirred solution of Compound 73-3 (600 mg, 1.273 mmol, 1 equiv) and Compound 57-9 (633.10 mg, 1.146 mmol, 0.9 equiv) in DCM (20 mL) was added Ti(Oi-Pr)₄ (1085.17 mg, 3.819 mmol, 3 equiv) at room temperature. The resulting mixture was stirred overnight at room temperature. To the above mixture was added STAB (809.21 mg, 3.819 mmol, 3 equiv) at room temperature. The resulting mixture was stirred for additional 6 h at room temperature. The reaction was quenched with 10% HOAc (aq.) (100 mL) at room temperature. The aqueous layer was extracted with EtOAc (50 mL). The aqueous layer was filtered, the filter cake was washed with water (10 mL). The filtrate was concentrated under reduced pressure. The residue was purified by reverse flash chromatography with the following conditions: column, C18 silica gel; mobile phase, MeCN in water (0.1% FA), 10% to 50% gradient in 20 min; detector, UV 254 nm. This resulted in Compound 73-4 (480 mg, 34.35%) as a yellow solid.

### Synthesis of Compound 73.

The Compound 73-4 (480 mg) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 60% B to 60% B in 11 min; Wave Length: 220/254 nm; RT1 (min): 5.19; RT2 (min): 6.88; the first peak is product) to afford the crude product. The crude product was purified by reverse flash chromatography with the following conditions: column, C18 silica gel; mobile phase, MeCN in water (0.1% FA), 10% to 30% gradient in 20 min; detector, UV 254 nm. This resulted in Compound 73 (137.2 mg, 28.24%) as a yellow solid.

LC-MS-Compound 73: (ES, m/z): [M−HCOOH+H]⁺ 1008

H-NMR-Compound 73: (400 MHz, DMSO-d6, δ ppm): 1.12-1.24 (m, 2H), 1.41-1.49 (m, 2H), 1.51-1.65 (m, 1H), 1.70-1.79 (m, 2H), 1.81-1.89 (m, 2H), 1.91-2.02 (m, 1H), 2.11-2.25 (m, 6H), 2.31-2.45 (m, 2H), 2.45-2.50 (m, 4H), 2.51-2.61 (m, 4H), 2.61-2.73 (m, 2H), 2.85-2.95 (m, 1H), 2.95-3.05 (m, 2H), 3.11-3.17 (m, 2H), 3.19 (s, 3H), 3.24 (s, 3H), 3.33-3.45 (m, 5H), 3.55-3.56 (m, 2H), 4.04-4.11 (m, 1H), 4.18-4.23 (m, 1H), 4.31-4.35 (m, 1H), 5.03-5.07 (m, 1H), 7.01-7.07 (m, 3H), 7.25-7.27 (d, 1H), 7.41 (s, 1H), 7.49-7.53 (m, 2H), 7.67-7.69 (m, 2H), 7.79 (s, 1H), 8.20 (s, 2H), 8.32 (s, 1H), 10.95 (s, 1H).

### Example 72. Compound 74

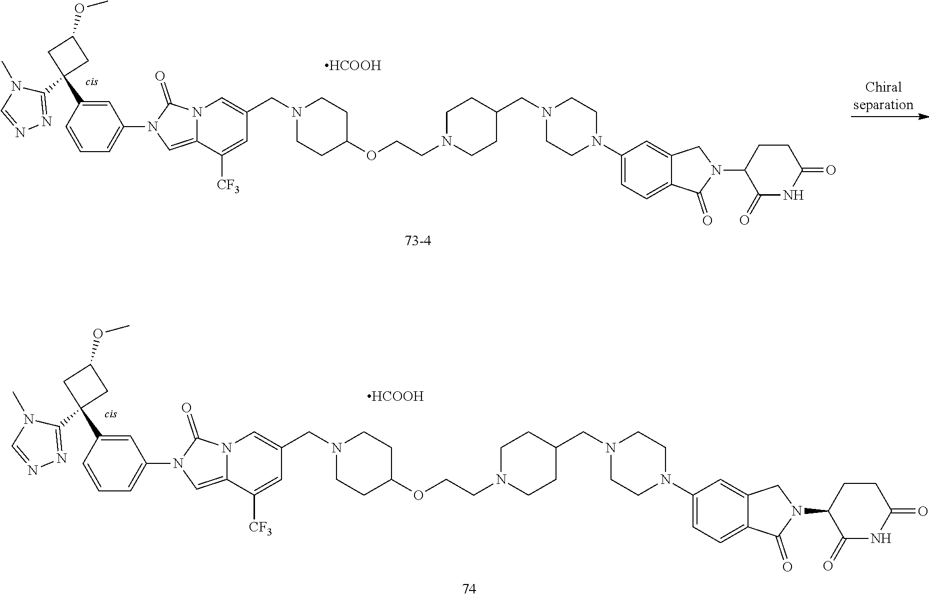 [SMILES: CO[C@H]1C[C@@](c2cccc(-n3cc4c(C(F)(F)F)cc(CN5CCC(OCCN6CCC(CN7CCN(c8ccc9c(c8)CN(C8CCC(=O)NC8=O)C9=O)CC7)CC6)CC5)cn4c3=O)c2)(c2nncn2C)C1 ; "·HCOOH" ; CO[C@H]1C[C@@](c2cccc(-n3cc4c(C(F)(F)F)cc(CN5CCC(OCCN6CCC(CN7CCN(c8ccc9c(c8)CN([C@H]8CCC(=O)NC8=O)C9=O)CC7)CC6)CC5)cn4c3=O)c2)(c2nncn2C)C1 ; "·HCOOH"]

### Synthesis of Compound 74.

The Compound 73-4 (480 mg) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 60% B to 60% B in 11 min; Wave Length: 220/254 nm; RT1 (min): 5.19; RT2 (min): 6.88; the second peak is product) to afford the crude product. The crude product was purified by reverse flash chromatography with the following conditions: column, C18 silica gel; mobile phase, MeCN in water (0.1% FA), 10% to 30% gradient in 20 min; detector, UV 254 nm. This resulted in Compound 74 (144.0 mg, 29.61%) as a yellow solid.

LC-MS-Compound 74: (ES, m/z): [M−HCOOH+H]⁺ 1008

H-NMR-Compound 74: (400 MHz, DMSO-d6, δ ppm): 1.12-1.24 (m, 2H), 1.41-1.49 (m, 2H), 1.51-1.65 (m, 1H), 1.70-1.79 (m, 2H), 1.81-1.89 (m, 2H), 1.91-2.02 (m, 1H), 2.11-2.25 (m, 6H), 2.31-2.45 (m, 2H), 2.45-2.50 (m, 4H), 2.51-2.73 (m, 6H), 2.85-2.95 (m, 1H), 2.95-3.05 (m, 2H), 3.11-3.17 (m, 2H), 3.19 (s, 3H), 3.24 (s, 3H), 3.33-3.45 (m, 5H), 3.55-3.56 (m, 2H), 4.04-4.11 (m, 1H), 4.18-4.23 (m, 1H), 4.31-4.35 (m, 1H), 5.03-5.07 (m, 1H), 7.01-7.07 (m, 3H), 7.25-7.27 (d, 1H), 7.41 (s, 1H), 7.49-7.53 (m, 2H), 7.67-7.69 (m, 2H), 7.79 (s, 1H), 8.20 (s, 2H), 8.32 (s, 1H), 10.95 (s, 1H).

### Example 73. Compound 75

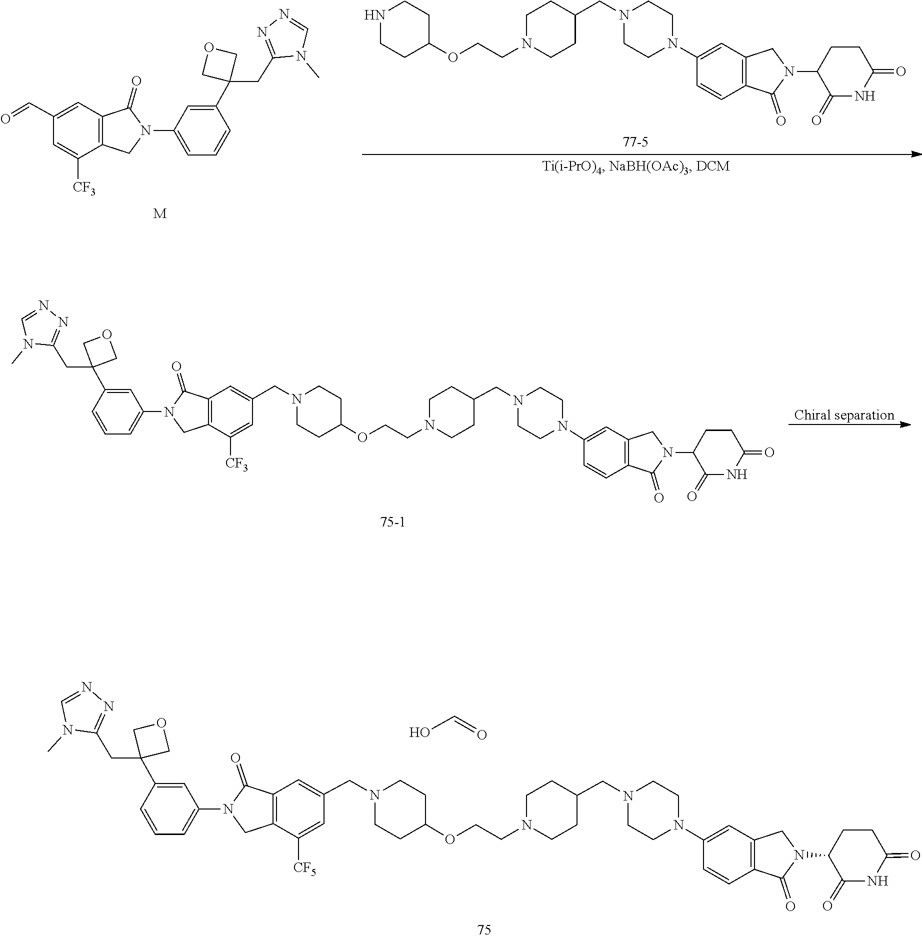

### Synthesis of Compound 75-1.

To a stirred solution of Intermediate M (880 mg, 1.928 mmol, 1 equiv) and Compound 77-5 (1065.66 mg, 1.928 mmol, 1 equiv) in DCM (20 mL) was added Ti(Oi-Pr)₄ (2191.93 mg, 7.712 mmol, 4 equiv). The resulting mixture was stirred for overnight at room temperature under nitrogen atmosphere. To the above mixture was added NaBH(OAc)₃ (1225.88 mg, 5.784 mmol, 3 equiv). The resulting mixture was stirred for additional 3 h at room temperature. The reaction was quenched by the addition of Water/HOAc=10/1 (150 mL) at room temperature. The aqueous layer was extracted with EtOAc (2×50 mL). The aqueous layer was concentrated under reduced pressure. The residue was purified by reverse flash chromatography with the following conditions: column, C18 silica gel; mobile phase, MeCN in Water (0.1% FA), 5% to 55% gradient in 30 min; detector, UV 254 nm. This resulted in Compound 75-1 (406 mg, 21.20%) as a yellow solid.

### Synthesis of Compound 75.

Compound 75-1 (406 mg) was separated by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 60% B to 60% B in 20 min; Wave Length: 220/254 nm; RT1 (min): 9.61; RT2 (min): 16.23; The first peak was product) to afford Compound 75 (175.2 mg, 41.24%) as a yellow solid.

LC-MS-Compound 75: (ES, m/z): [M−HCOOH+H]⁺ 993

H-NMR-Compound 75: (400 MHz, DMSO-d6, δ ppm): 1.16-1.24 (m, 3H), 1.45-1.48 (m, 2H), 1.52-1.61 (m, 1H), 1.70-1.74 (m, 2H), 1.83-1.85 (m, 2H), 1.95-1.98 (m, 1H), 2.11-2.22 (m, 6H), 2.31-2.40 (m, 1H), 2.53-2.50 (m, 5H), 2.51-2.66 (m, 5H), 2.82-2.92 (m, 4H), 2.97-3.03 (m, 2H), 3.27-3.30 (m, 5H), 3.51-3.57 (m, 2H), 3.68 (s, 2H), 4.18-4.22 (d, 1H), 4.31-4.35 (d, 1H), 4.88-4.90 (m, 2H), 4.96-4.98 (m, 2H), 5.03-5.07 (m, 1H), 5.10 (s, 2H), 6.77-6.78 (d, 1H), 7.05-7.07 (m, 2H), 7.33-7.37 (t, 1H), 7.40 (s, 1H), 7.51-7.53 (m, 2H), 7.87-7.89 (m, 1H), 7.93 (s, 1H), 7.98 (s, 1H), 8.19 (s, 3H), 10.95 (s, 1H).

### Example 74. Compound 76

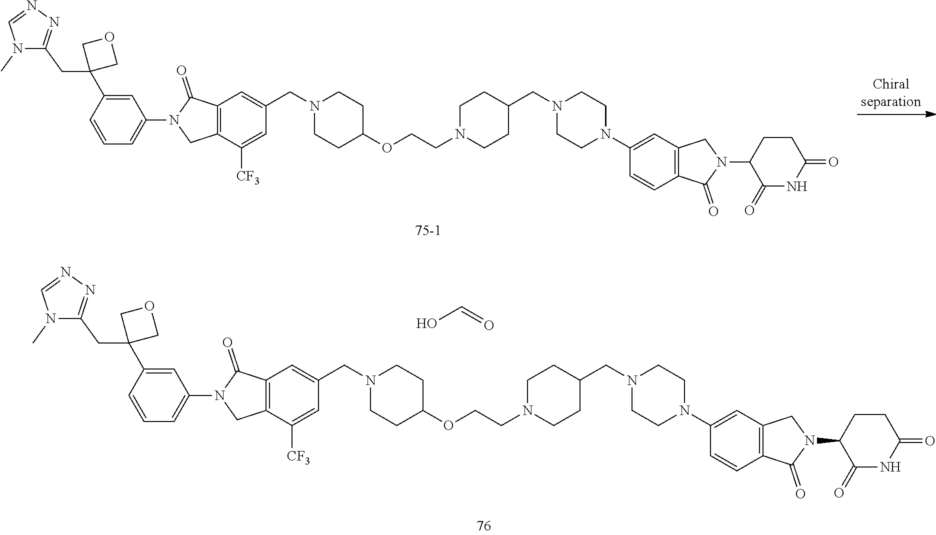 [SMILES: Cn1cnnc1CC1(c2cccc(N3Cc4c(cc(CN5CCC(OCCN6CCC(CN7CCN(c8ccc9c(c8)CN(C8CCC(=O)NC8=O)C9=O)CC7)CC6)CC5)cc4C(F)(F)F)C3=O)c2)COC1 ; Cn1cnnc1CC1(c2cccc(N3Cc4c(cc(CN5CCC(OCCN6CCC(CN7CCN(c8ccc9c(c8)CN([C@H]8CCC(=O)NC8=O)C9=O)CC7)CC6)CC5)cc4C(F)(F)F)C3=O)c2)COC1 ; O=CO]

### Synthesis of Compound 76.

Compound 75-1 (406 mg) was purified by Prep-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 60% B to 60% B in 20 min; Wave Length: 220/254 nm; RT1 (min): 9.61; RT2 (min): 16.23; the second peak was product) to afford Compound 76 (202.0 mg, 47.55%) as a yellow solid.

LC-MS-Compound 76: (ES, m/z): [M−HCOOH+H]⁺ 993

H-NMR-Compound 76: (400 MHz, DMSO-d6, δ ppm): 1.18-1.26 (m, 3H), 1.45-1.48 (m, 2H), 1.52-1.61 (m, 1H), 1.72-1.75 (m, 2H), 1.83-1.86 (m, 2H), 1.95-1.98 (m, 1H), 2.11-2.18 (m, 4H), 2.25-2.32 (m, 2H), 2.33-2.40 (m, 1H), 2.48-2.50 (m, 5H), 2.54-2.66 (m, 1H), 2.67-2.74 (m, 4H), 2.84-2.92 (m, 4H), 3.03-3.06 (m, 2H), 3.27-3.34 (m, 4H), 3.34-3.36 (m, 1H), 3.56-3.59 (m, 2H), 3.69 (s, 2H), 4.18-4.22 (d, 1H), 4.31-4.35 (d, 1H), 4.88-4.90 (m, 2H), 4.96-4.98 (m, 2H), 5.03-5.10 (m, 2H), 6.77-6.78 (d, 1H), 7.05-7.07 (m, 2H), 7.33-7.37 (t, 1H), 7.40 (s, 1H), 7.51-7.53 (m, 1H), 7.87-7.89 (m, 1H), 7.93 (s, 1H), 7.98 (s, 2H), 8.19 (s, 4H), 10.95 (s, 1H).

### Example 75. Compound 77

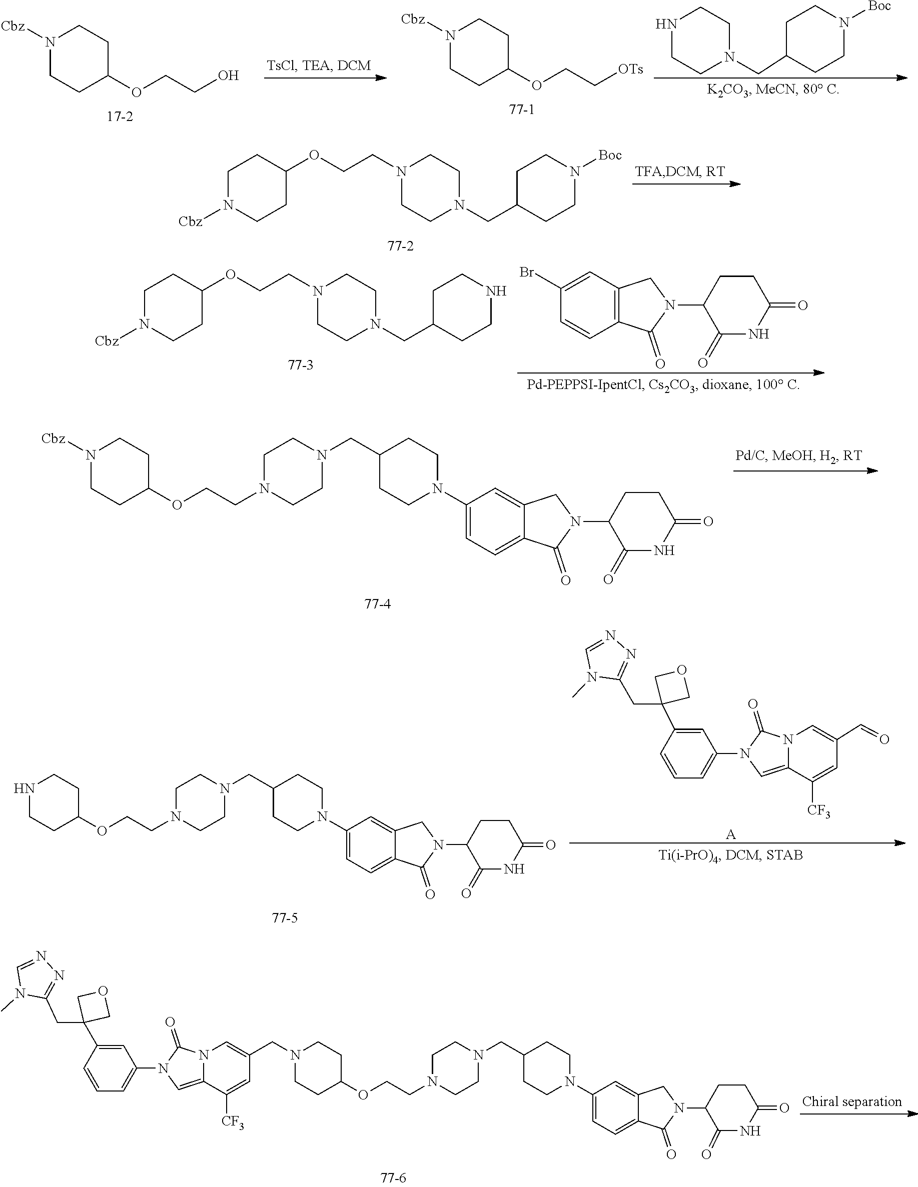

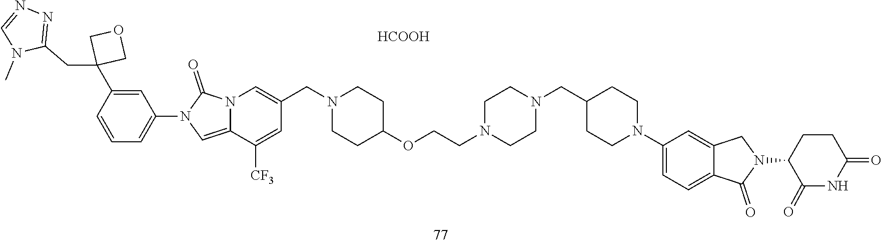 [SMILES: Cn1cnnc1CC1(c2cccc(-n3cc4c(C(F)(F)F)cc(CN5CCC(OCCN6CCN(CC7CCN(c8ccc9c(c8)CN([C@@H]8CCC(=O)NC8=O)C9=O)CC7)CC6)CC5)cn4c3=O)c2)COC1 ; "HCOOH"]

### Synthesis of Compound 77-1.

A solution of benzyl Compound 17-2 (20 g, 71.59 mmol, 10.0 equiv) and TEA (21.7 g, 214.79 mmol, 3.0 equiv), TsCl (20.5 g, 107.39 mmol, 1.5 equiv) in DCM (200 mL) was stirred for overnight at room temperature under nitrogen atmosphere. The reaction was quenched with sat. NH₄Cl (aq.) (500 mL) at room temperature. The aqueous layer was extracted with CH₂Cl₂ (6×300 mL). The resulting mixture was concentrated under vacuum. The residue was purified by silica gel column chromatography, eluted with PE/EA (5:1) to afford Compound 77-1 (15 g, 48%) as a yellow oil.

### 2. Synthesis of Compound 77-2.

To a stirred solution of Compound 51-1 (15 g, 34.60 mmol, 1.0 equiv) and tert-butyl 4-(piperazin-1-ylmethyl) piperidine-1-carboxylate (9.8 g, 34.60 mmol, 1.0 equiv) in MeCN (150 mL) were added K₂CO₃ (14.3 g, 103.80 mmol, 3.0 equiv) at room temperature under nitrogen atmosphere. The resulting mixture was stirred for overnight at 80° C. under nitrogen atmosphere. The reaction was quenched with water (500 mL) at room temperature. The aqueous layer was extracted with CH₂C₂ (3×200 mL). The resulting mixture was concentrated under reduced pressure. The residue was purified by silica gel column chromatography, eluted with CH₂Cl₂/MeOH (90:1) to afford Compound 51-2 (12 g, 59%) as a colorless oil.

### Synthesis of Compound 77-3.

To a stirred solution of Compound 77-2 (12 g, 22.02 mmol, 1.0 equiv) in DCM (120 mL) were added TFA (24 mL) at room temperature under nitrogen atmosphere. The resulting mixture was stirred for overnight at room temperature under nitrogen atmosphere. The mixture was basified to pH 8 with NH₃·H₂O. The residue was purified by silica gel column chromatography, eluted with CH₂Cl₂/MeOH (10:1) to afford Compound 77-3 (7.4 g, 70%) as a colorless oil.

### Synthesis of Compound 77-4.

To a stirred solution of Compound 77-3 (7.3 g, 16.41 mmol, 1.0 equiv) and 3-(5-bromo-1-oxo-3H-isoindol-2-yl) piperidine-2,6-dione (10.6 g, 32.83 mmol, 2.0 equiv) in 1,4-dioxane (73 mL) were added Cs₂CO₃ (16 g, 49.25 mmol, 3.0 equiv) and Pd-PEPPSI-IPentCl 2-methylpyridine (o-picoline) (1.5 g, 1.64 mmol, 0.1 equiv) at room temperature under nitrogen atmosphere. The resulting mixture was stirred for overnight at 100° C. under nitrogen atmosphere. The reaction was quenched by the addition of 10% AcOH (500 mL) at room temperature. The aqueous layer was extracted with MeOH:CH₂Cl₂ (1:10) (5×400 mL). The resulting mixture was concentrated under reduced pressure. The residue was purified by silica gel column chromatography, eluted with CH₂Cl₂/MeOH (20:1) to afford Compound 77-4 (2 g, 18%) as an off-white solid.

### Synthesis of Compound 77-5.

To a solution of Compound 77-4 (2 g, 2.91 mmol, 1 equiv) in 60 mL MeOH was added Pd/C (10%, 600 mg) under nitrogen atmosphere in a 10 mL round-bottom flask. The mixture was hydrogenated at room temperature for 2 h under hydrogen atmosphere using a hydrogen balloon, filtered through a Celite pad and concentrated under reduced pressure. This resulted in Compound 77-5 (1.5 g, 93%) as a colorless oil.

### Synthesis of Compound 77-6.

To a stirred solution of Compound 77-5 (700 mg, 1.26 mmol, 1.0 equiv) and Intermediate A (868 mg, 1.89 mmol, 1.5 equiv) in DCM (7 mL) was added Ti(Oi-Pr)₄ (1439 mg, 5.06 mmol, 4.0 equiv) at room temperature. The resulting mixture was stirred for overnight at room temperature. To the above mixture was added STAB (805 mg, 3.79 mmol, 3.0 equiv) at room temperature. The resulting mixture was stirred for additional 2 h at room temperature. The reaction was quenched by the addition of 10% AcOH (100 mL) at room temperature. The aqueous layer was extracted with EtOAc (2×60 mL). The aqueous phase was concentrated under reduced pressure. The residue was purified by reversed-phase flash chromatography with the following conditions: column, C18 silica gel; mobile phase, MeCN in water (0.1% FA), 10% to 50% gradient in 30 min; detector, UV 254 nm. The crude product (500 mg) was purified by Prep-HPLC with the following conditions (Column: Xselect CSH C18 OBD Column 30*150 mm 5 μm, n; Mobile Phase A: water (0.1% FA), Mobile Phase B: ACN; Flow rate: 60 mL/min; Gradient: 6% B to 17% B in 9 min, 17% B; Wave Length: 254; 220 nm; RT1 (min): 8.98) to afford Compound 77-6 (350 mg, 28%) as a yellow solid.

### Synthesis of Compound 77.

The Compound 77-6 (350 mg) was purified by Prep-CHIRAL-HPLC with the following conditions Column: CHIRALPAK IH, 2*25 cm, 5 μm; Mobile Phase A: Hex (0.2% TEA)—HPLC, Mobile Phase B: DCM:ACN=1:1—HPLC; Flow rate: 20 mL/min; Gradient: 60% B to 60% B in 28 min; Wave Length: 220/254 nm; RT1 (min): 11.91; RT2 (min): 16.96; Sample Solvent: DCM:ACN=1:1—HPLC; Injection Volume: 0.3 mL; Number of Runs: 4. The first peak was the product. The residue was purified by reversed-phase flash chromatography with the following conditions: column, C18 silica gel; mobile phase, MeCN in water (0.1% FA), 10% to 50% gradient in 30 min; detector, UV 254 nm. This resulted in Compound 77 (92 mg, 24%) as a yellow solid.

LC-MS-Compound 77: (ES, m/z): [M-COOH]⁺ 994

H-NMR-Compound 77: (400 MHz, DMSO-d6, δ ppm): 1.16-1.21 (m, 2H), 1.44-1.46 (m, 2H), 1.74-1.80 (m, 5H), 1.95-1.98 (m, 1H), 2.11-2.21 (m, 4H), 2.34-2.50 (m, 10H), 2.67-2.75 (m, 4H), 2.80-2.90 (m, 3H), 2.97 (s, 3H), 3.48-3.53 (m, 3H), 3.51-3.55 (m, 2H), 3.56-3.58 (m, 2H), 3.85-3.88 (m, 2H), 4.17-4.21 (d, 1H), 4.29-4.34 (d, 1H), 4.91-5.05 (m, 5H), 6.88-6.90 (d, 1H), 7.01-7.04 (m, 3H), 7.33 (s, 1H), 7.38-7.42 (m, 2H), 7.48-7.51 (d, 1H), 7.67 (s, 1H), 7.75-7.77 (m, 1H), 8.21 (s, 1H), 10.95 (s, 1H).

### Example 76. Compound 78

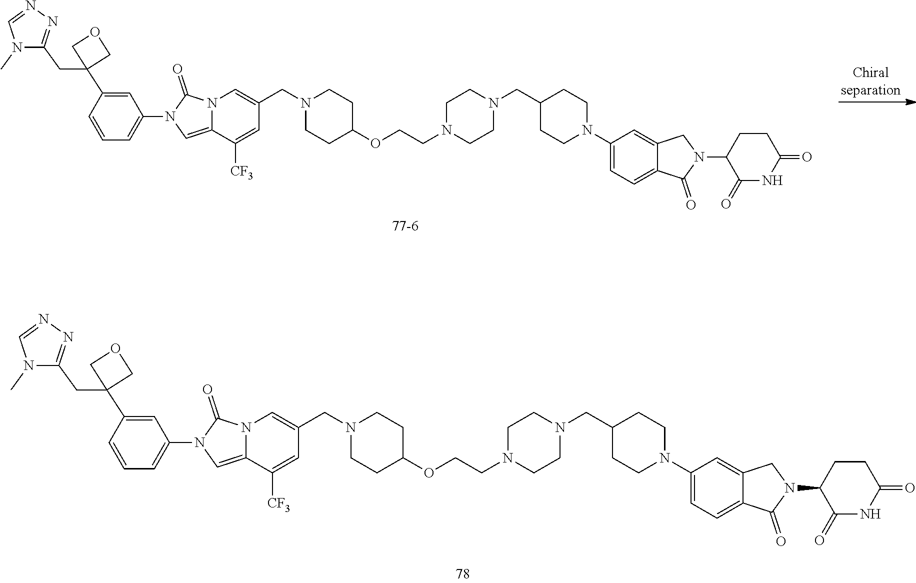 [SMILES: Cn1cnnc1CC1(c2cccc(-n3cc4c(C(F)(F)F)cc(CN5CCC(OCCN6CCN(CC7CCN(c8ccc9c(c8)CN(C8CCC(=O)NC8=O)C9=O)CC7)CC6)CC5)cn4c3=O)c2)COC1 ; Cn1cnnc1CC1(c2cccc(-n3cc4c(C(F)(F)F)cc(CN5CCC(OCCN6CCN(CC7CCN(c8ccc9c(c8)CN([C@H]8CCC(=O)NC8=O)C9=O)CC7)CC6)CC5)cn4c3=O)c2)COC1]

### Synthesis of Compound 78.

The Compound 77-6 (350 mg) was purified by Prep-CHIRAL-HPLC with the following conditions Column: CHIRALPAK IH, 2*25 cm, 5 μm; Mobile Phase A: Hex (0.2% TEA)—HPLC, Mobile Phase B: DCM:ACN=1:1—HPLC; Flow rate: 20 mL/min; Gradient: 60% B to 60% B in 28 min; Wave Length: 220/254 nm; RT1 (min): 11.91; RT2 (min): 16.96; Sample Solvent: DCM:ACN=1:1—HPLC; Injection Volume: 0.3 mL; Number of Runs: 4. The second peak was the product. This resulted in Compound 78 (68 mg, 19%) as a yellow solid.

LC-MS-Compound 78: (ES, m/z): [M+H]⁺ 994

H-NMR-Compound 78: (400 MHz, DMSO-d6, δ ppm): 1.08-1.16 (m, 2H), 1.42-1.46 (m, 2H), 1.74-1.81 (m, 5H), 1.91-1.95 (m, 1H), 2.11-2.16 (m, 4H), 2.34-2.51 (m, 10H), 2.61-2.67 (m, 4H), 2.80-2.90 (m, 3H), 2.97 (s, 3H), 3.48-3.53 (m, 3H), 3.51-3.55 (m, 2H), 3.56-3.58 (m, 2H), 3.85-3.88 (m, 2H), 4.17-4.21 (d, 1H), 4.29-4.34 (d, 1H), 4.91-5.05 (m, 5H), 6.88-6.90 (d, 1H), 7.01-7.04 (m, 3H), 7.33 (s, 1H), 7.38-7.42 (m, 2H), 7.48-7.51 (d, 1H), 7.67 (s, 1H), 7.75-7.77 (m, 1H), 8.21 (s, 1H), 10.95 (s, 1H).

### Example 77. Compound 79

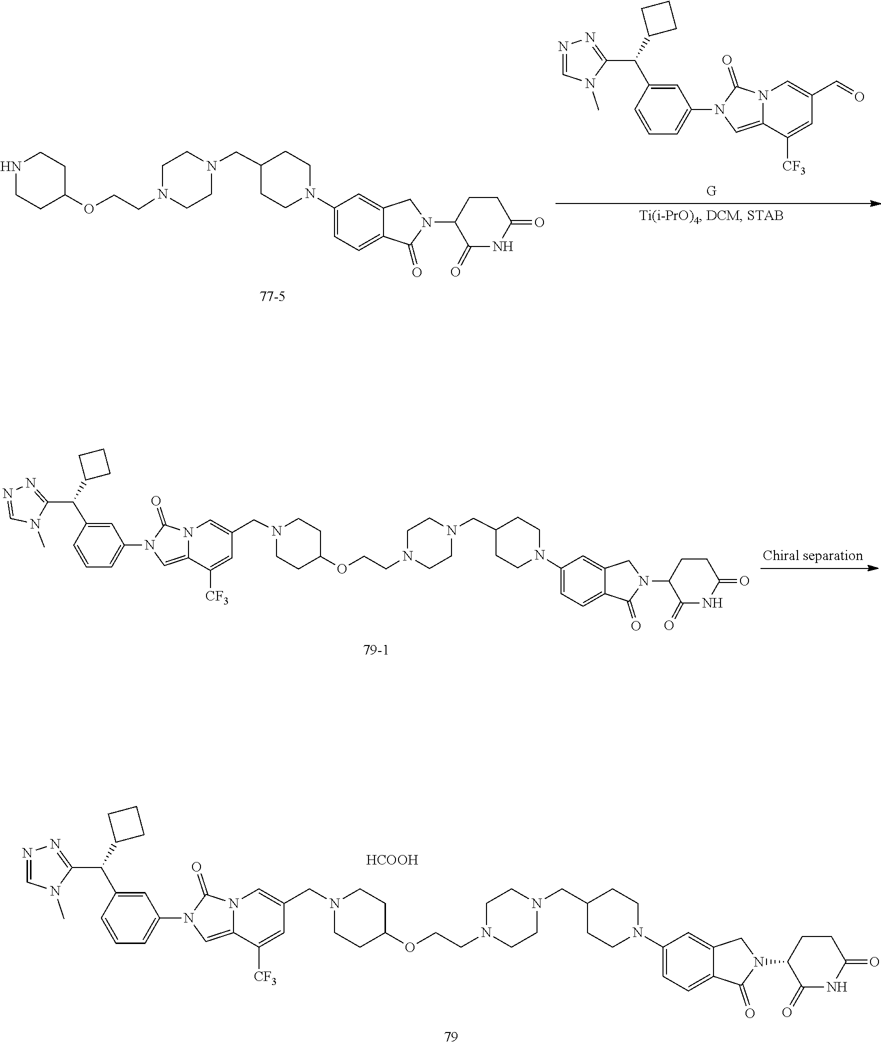 [SMILES: O=C1CCC(N2Cc3cc(N4CCC(CN5CCN(CCOC6CCNCC6)CC5)CC4)ccc3C2=O)C(=O)N1 ; Cn1cnnc1[C@@H](c1cccc(-n2cc3c(C(F)(F)F)cc(C=O)cn3c2=O)c1)C1CCC1 ; Cn1cnnc1[C@@H](c1cccc(-n2cc3c(C(F)(F)F)cc(CN4CCC(OCCN5CCN(CC6CCN(c7ccc8c(c7)CN(C7CCC(=O)NC7=O)C8=O)CC6)CC5)CC4)cn3c2=O)c1)C1CCC1 ; Cn1cnnc1[C@@H](c1cccc(-n2cc3c(C(F)(F)F)cc(CN4CCC(OCCN5CCN(CC6CCN(c7ccc8c(c7)CN([C@@H]7CCC(=O)NC7=O)C8=O)CC6)CC5)CC4)cn3c2=O)c1)C1CCC1 ; "HCOOH"]

### Synthesis of Compound 79-1.

To a stirred solution of Compound 77-5 (700 mg, 1.26 mmol, 1.0 equiv) and Intermediate G (865 mg, 1.89 mmol, 1.5 equiv) in DCM (7 mL) was added Ti(Oi-Pr)₄ (1439 mg, 5.06 mmol, 4.0 equiv) at room temperature. The resulting mixture was stirred for overnight at room temperature. To the above mixture was added STAB (805 mg, 3.79 mmol, 3.0 equiv) at room temperature. The resulting mixture was stirred for additional 2 h at room temperature. The reaction was quenched by the addition of 10% AcOH (100 mL) at room temperature. The aqueous layer was extracted with EtOAc (2×60 mL). The aqueous phase was concentrated under reduced pressure. The residue was purified by reversed-phase flash chromatography with the following conditions: column, C18 silica gel; mobile phase, MeCN in water (0.1% FA), 10% to 50% gradient in 30 min; detector, UV 254 nm. The crude product (250 mg) was purified by Prep-HPLC with the following conditions (Column: Sunfire prep C18 column, 30*150 mm, 5 μm; Mobile Phase A: Water (0.1% FA), Mobile Phase B: ACN; Flow rate: 60 mL/min; Gradient: 10% B to 21% B in 8 min, 21% B; Wave Length: 254; 220 nm; RT1 (min): 7.75) to afford Compound 79-1 (150 mg, 12%) as a yellow solid.

### Synthesis of Compound 79.

The Compound 79-1 (150 mg) was purified by Prep-CHIRAL-HPLC with the following conditions (Column: CHIRALPAK IH, 2*25 cm, 5 μm; Mobile Phase A: Hex (0.2% TEA)—HPLC, Mobile Phase B: DCM:ACN=1:1—HPLC; Flow rate: 20 mL/min; Gradient: 60% B to 60% B in 13 min; Wave Length: 220/254 nm; RT1 (min): 6.62; RT2 (min): 10.89; Sample Solvent: DCM:ACN=1:1—HPLC; Injection Volume: 1.5 mL; Number of Runs: 4) The first peak was the product. The residue was purified by reversed-phase flash chromatography with the following conditions: column, C18 silica gel; mobile phase, MeCN in water (0.1% FA), 10% to 50% gradient in 30 min; detector, UV 254 nm. This resulted in Compound 79 (67 mg, 42%) as a yellow solid.

LC-MS-Compound 79: (ES, m/z): [M-COOH]⁺ 992

H-NMR-Compound 79: (400 MHz, DMSO-d6, δ ppm): 1.06-1.09 (m, 2H), 1.10-1.16 (m, 1H), 1.42-1.44 (m, 2H), 1.70-1.81 (m, 1OH), 1.95-2.12 (m, 7H), 2.34-2.45 (m, 9H), 2.51-2.67 (m, 3H), 2.80-2.87 (m, 3H), 2.91-2.95 (m, 1H), 3.28-3.31 (m, 4H), 3.43 (s, 3H), 3.48-3.51 (m, 2H), 3.84-3.87 (m, 2H), 4.17-4.34 (m, 3H), 5.01-5.09 (m, 1H), 7.01-7.04 (d, 3H), 7.18-7.20 (d, 1H), 7.32 (s, 1H), 7.42-7.51 (m, 2H), 7.66-7.77 (m, 3H), 8.21 (s, 1H), 8.33 (s, 1H), 10.95 (s, 1H).

### Example 78. Compound 80

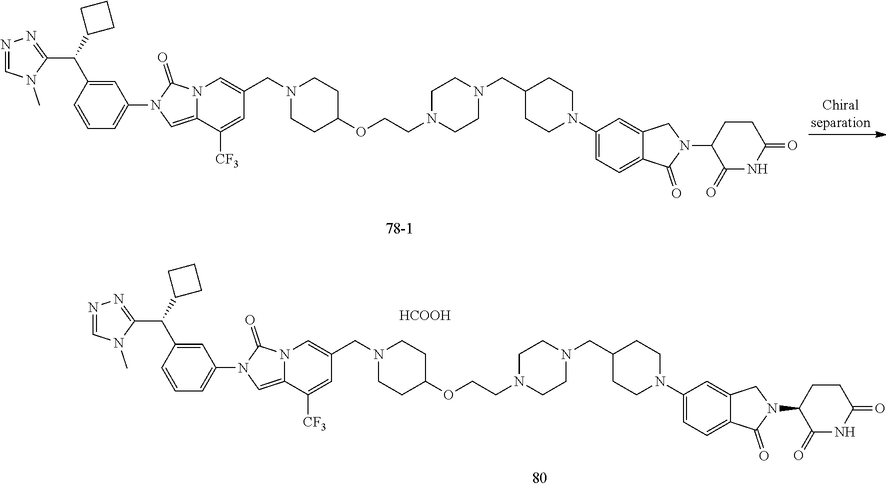 [SMILES: Cn1cnnc1[C@@H](c1cccc(-n2cc3c(C(F)(F)F)cc(CN4CCC(OCCN5CCN(CC6CCN(c7ccc8c(c7)CN(C7CCC(=O)NC7=O)C8=O)CC6)CC5)CC4)cn3c2=O)c1)C1CCC1 ; Cn1cnnc1[C@@H](c1cccc(-n2cc3c(C(F)(F)F)cc(CN4CCC(OCCN5CCN(CC6CCN(c7ccc8c(c7)CN([C@H]7CCC(=O)NC7=O)C8=O)CC6)CC5)CC4)cn3c2=O)c1)C1CCC1 ; "HCOOH"]

### Synthesis of Compound 80.

The Compound 79-1 (150 mg) was purified by Prep-CHIRAL-HPLC with the following conditions (Column: CHIRALPAK IH, 2*25 cm, 5 μm; Mobile Phase A: Hex (0.2% TEA)—HPLC, Mobile Phase B: DCM:ACN=1:1—HPLC; Flow rate: 20 mL/min; Gradient: 60% B to 60% B in 13 min; Wave Length: 220/254 nm; RT1 (min): 6.62; RT2 (min): 10.89; Sample Solvent: DCM:ACN=1:1—HPLC; Injection Volume: 1.5 mL; Number of Runs: 4). The second peak was the product. The residue was purified by reversed-phase flash chromatography with the following conditions: column, C18 silica gel; mobile phase, MeCN in water (0.1% FA), 10% to 50% gradient in 30 min; detector, UV 254 nm. This resulted in Compound 80 (57 mg, 38%) as a yellow solid.

LC-MS-Compound 80: (ES, m/z): [M-COOH]⁺ 992

H-NMR-Compound 80: (400 MHz, DMSO-d6, δ ppm): 1.06-1.09 (m, 2H), 1.10-1.16 (m, 1H), 1.42-1.44 (m, 2H), 1.70-1.81 (m, 10H), 1.95-2.12 (m, 7H), 2.34-2.45 (m, 9H), 2.51-2.67 (m, 3H), 2.80-2.87 (m, 3H), 2.91-2.95 (m, 1H), 3.28-3.31 (m, 4H), 3.43 (s, 3H), 3.48-3.51 (m, 2H), 3.84-3.87 (m, 2H), 4.17-4.34 (m, 3H), 5.01-5.09 (m, 1H), 7.01-7.04 (d, 3H), 7.18-7.20 (d, 1H), 7.32 (s, 1H), 7.42-7.51 (m, 2H), 7.66-7.77 (m, 3H), 8.21 (s, 1H), 8.33 (s, 1H), 10.95 (s, 1H).

### Example 79. Compound 81

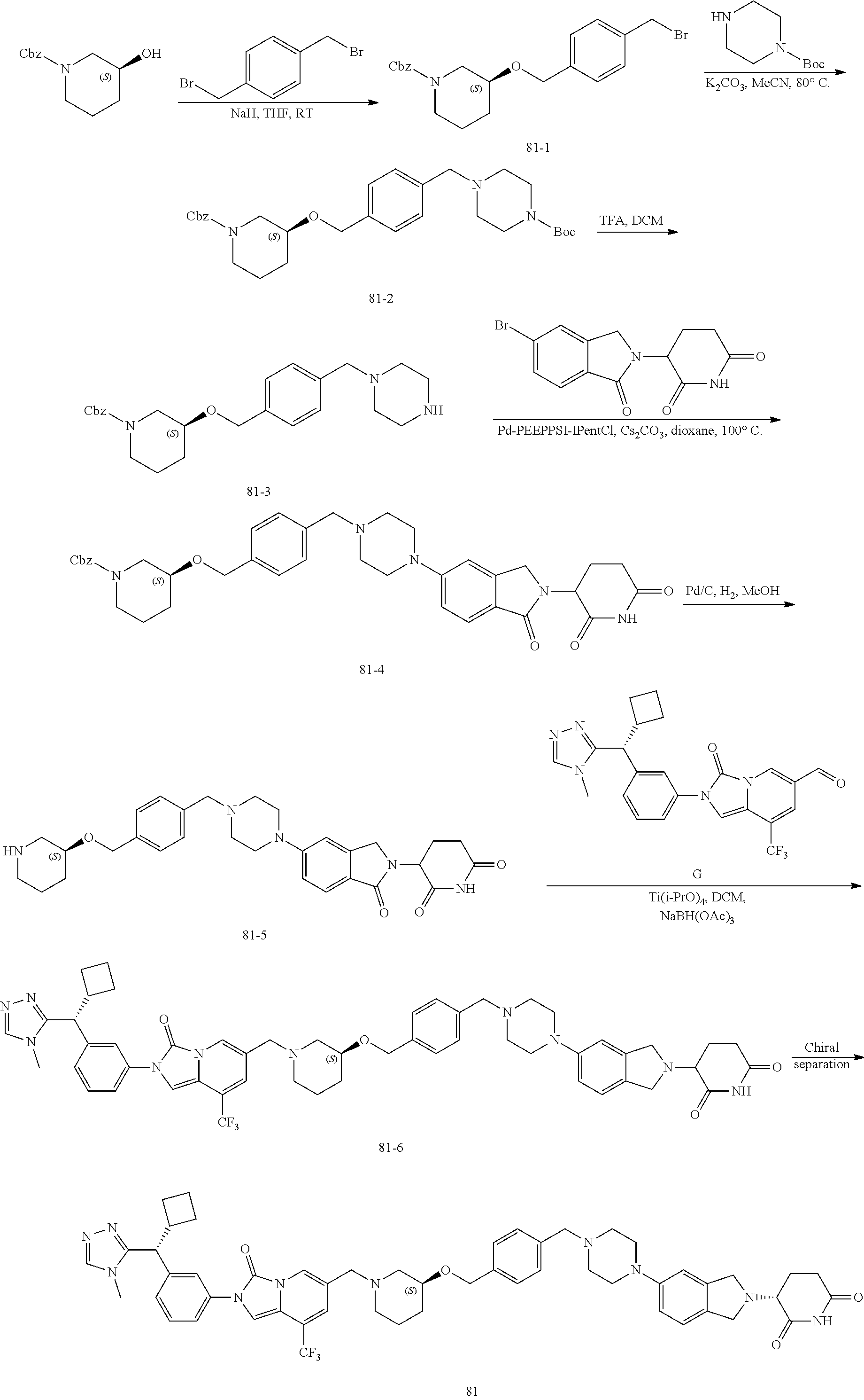

### Synthesis of Compound 81-1.

To a solution of benzyl (3S)-3-hydroxypiperidine-1-carboxylate (10.0 g, 42.50 mmol, 1.0 equiv) in THF (200 mL) was added sodium hydride (1.7 g, 42.50 mmol, 1.0 equiv, 60% in oil) at 0 degrees C. The mixture was stirred for 30 min. To the above mixture 1,4-bis(bromomethyl)benzene (13.46 g, 51.002 mmol, 1.2 equiv) was added. The resulting mixture was stirred for overnight at room temperature. The reaction was quenched with sat. NH₄Cl (aq.) (600 mL) at room temperature. The aqueous layer was extracted with EtOAc (2×200 mL). The resulting mixture was concentrated under reduced pressure. The residue was purified by silica gel column chromatography, eluted with PE/EA=1:1 to afford Compound 81-1 (9.0 g, 47%) as a green oil.

### Synthesis of Compound 81-2.

To a stirred solution of Compound 81-1 (10.0 g, 23.91 mmol, 1.0 equiv) and tert-butyl piperazine-1-carboxylate (6.7 g, 35.86 mmol, 1.5 equiv) in MeCN (120 mL) was added K₂CO₃ (3.9 g, 28.67 mmol, 1.2 equiv) at room temperature under nitrogen atmosphere. The resulting mixture was stirred for overnight at 80° C. under nitrogen atmosphere. The reaction was quenched by the addition of sat. NH₄Cl (aq.) (500 mL) at room temperature. The aqueous layer was extracted with CH₂Cl₂ (3×200 mL). The resulting mixture was concentrated under reduced pressure. The residue was purified by silica gel column chromatography, eluted with PE/EA=2:1 to afford Compound 81-2 (7.7 g, 58%) as a yellow green oil.

### Synthesis of Compound 81-3.

To a stirred solution of Compound 81-2 (7.7 g, 14.70 mmol, 1.0 equiv) in DCM (58 mL) was added TFA (19 mL) at room temperature under nitrogen atmosphere. The resulting mixture was stirred for overnight at room temperature under nitrogen atmosphere. The resulting mixture was concentrated under reduced pressure. The mixture was neutralized to pH 7 with saturated Na₂CO₃ (aq.). The aqueous layer was extracted with CH₂Cl₂ (3×200 mL). The resulting mixture was concentrated under reduced pressure. The crude product was used in the next step directly without further purification. This resulted in Compound 81-3 (5.2 g, 79%) as a brown oil.

### Synthesis of Compound 81-4.

To a stirred solution of Compound 81-3 (5.0 g, 11.80 mmol, 1.0 equiv) and 3-(5-bromo-1-oxo-3H-isoindol-2-yl)piperidine-22,6-dion (7.6 g, 23.61 mmol, 2.0 equiv) in 1,4-dioxane (50 mL) were added Cs₂CO₃ (11.5 g, 35.41 mmol, 3.0 equiv) and Pd-PEPPSI-IPentCl 2-methylpyridine (o-picoline (990 mg, 1.18 mmol, 0.1 equiv) at room temperature under nitrogen atmosphere. The resulting mixture was stirred for overnight at 100° C. under nitrogen atmosphere. The reaction was quenched by the addition of 10% Water/HOAc (300 mL) at room temperature. The aqueous layer was extracted with MeOH/CH₂Cl₂=1:10 (3×300 mL). The resulting mixture was concentrated under reduced pressure. The residue was purified by silica gel column chromatography, eluted with CH₂Cl₂/MeOH (10:1) to afford Compound 81-4 (3.0 g, 38%) as a off-white solid.

### Synthesis of Compound 81-5.

To a solution of Compound 81-4 (3.0 g, 4.50 mmol, 1.0 equiv) in 90 mL EtOAc and 18 mL AcOH was added Pd/C (10%, 900 mg) under nitrogen atmosphere in a 250 mL round-bottom flask. The mixture was hydrogenated at room temperature for 2 h under hydrogen atmosphere using a hydrogen balloon, filtered through a Celite pad and concentrated under reduced pressure. This resulted in Compound 81-5 (2.2 g, 91%) as a colorless oil.

### Synthesis of Compound 81-6.

To a stirred solution of Compound 81-5 (1.0 g, 1.88 mmol, 1.0 equiv) and Intermediate G (860 mg, 1.88 mmol, 1.0 equiv) in DCM (20 mL) was added Ti(Oi-Pr)₄ (2.1 g, 7.52 mmol, 4.0 equiv). The resulting mixture was stirred for overnight at room temperature under nitrogen atmosphere. To the above mixture was added NaBH(OAc)₃ (1.2 g, 5.64 mmol, 3.0 equiv). The resulting mixture was stirred for additional 4 h at room temperature. The reaction was quenched by the addition of water/HOAc=10:1 (100 mL). The resulting mixture was extracted with CH₂Cl₂/MeOH=10:1 (3×50 mL). The combined organic layers were concentrated under reduced pressure. The residue was purified by reversed-phase flash chromatography with the following conditions: column, C18 silica gel; mobile phase, MeCN in Water (0.1% FA), 15% to 65% gradient in 30 min; detector, UV 254 nm. This resulted in Compound 81-6 (400 mg, 21%) as a yellow solid.

### Synthesis of Compound 81.

The Compound 81-6 (400 mg, 0.41 mmol, 1.0 equiv) was purified by Prep-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 60% B to 60% B in 13 min; Wave Length: 220/254 nm; RT1 (min): 6.01; RT2 (min): 9.97; the first peak was product) to afford Compound 81 (129 mg, 32%) as a yellow solid.

LCMS-Compound 81: (ES, m/z): [M+H] 971

NMR-Compound 81: (400 MHz, DMSO-d6, δ ppm): 1.21-1.25 (m, 2H), 1.67-1.69 (m, 1H), 1.71-1.85 (m, 6H), 1.94-2.08 (m, 5H), 2.34-2.50 (m, 1H), 2.50-2.51 (m, 2H), 2.51-2.61 (m, 2H), 2.87-2.90 (m, 2H), 3.23-3.33 (m, 7H), 3.36-3.49 (m, 7H), 4.17-4.30 (m, 3H), 4.34-4.49 (m, 2H), 5.02-5.07 (m, 1H), 7.01-7.05 (m, 3H), 7.18-7.20 (d, 1H), 7.27-7.33 (m, 4H), 7.42-7.44 (t, 1H), 7.46-7.50 (d, 2H), 7.67-7.75 (m, 3H), 8.33 (s, 1H), 10.95 (s, 1H).

### Example 80. Compound 82

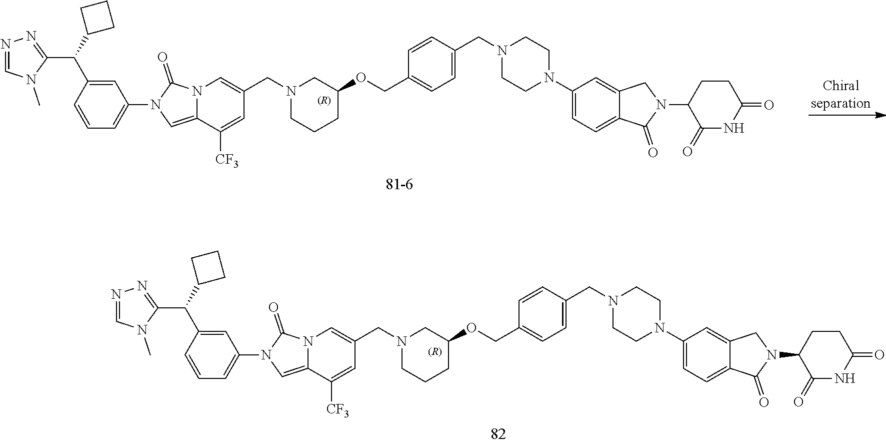

### Synthesis of Compound 82.

The Compound 81-6 (400 mg, 0.41 mmol, 1.0 equiv) was purified by Prep-Chiral-HPLC with the following conditions (Column: CHIRALPAK IH, 2*25 cm, 5 μm, Mobile Phase A: MTBE (MTBE (0.1% TEA), Mobile Phase B: DCM:ACN=1:1; Flow rate: 20 mL/min; Gradient: 60% B to 60% B in 13 min; Wave Length: 220/254 nm; RT1 (min): 6.01; RT2 (min): 9.97; the second peak was product) to afford Compound 82 (132 mg, 32%) as a yellow solid.

LCMS-Compound 82: (ES, m/z): [M+H]⁺ 973

NMR-Compound 82: 1.51-1.54 (m, 2H), 1.22-1.25 (m, 1H), 1.67-1.70 (d, 1H), 1.72-1.94 (m, 6H), 1.95-2.08 (m, 5H), 2.50-2.5 (m, 1H), 2.51-2.61 (m, 2H), 2.87-2.90 (m, 2H), 3.27-3.33 (m, 2H), 3.33-3.38 (m, 7H), 3.43-3.49 (m, 7H), 4.17-4.34 (m, 3H), 4.48-4.49 (m, 2H), 5.02-5.06 (d, 1H), 7.01-7.05 (m, 3H), 7.18-7.20 (d, 1H), 7.27-7.33 (m, 4H), 7.42-7.44 (t, 1H), 7.46-7.52 (d, 2H), 7.67-7.75 (m, 3H), 8.33 (s, 1H), 10.95 (s, 1H).

### Example 81. Compound 83

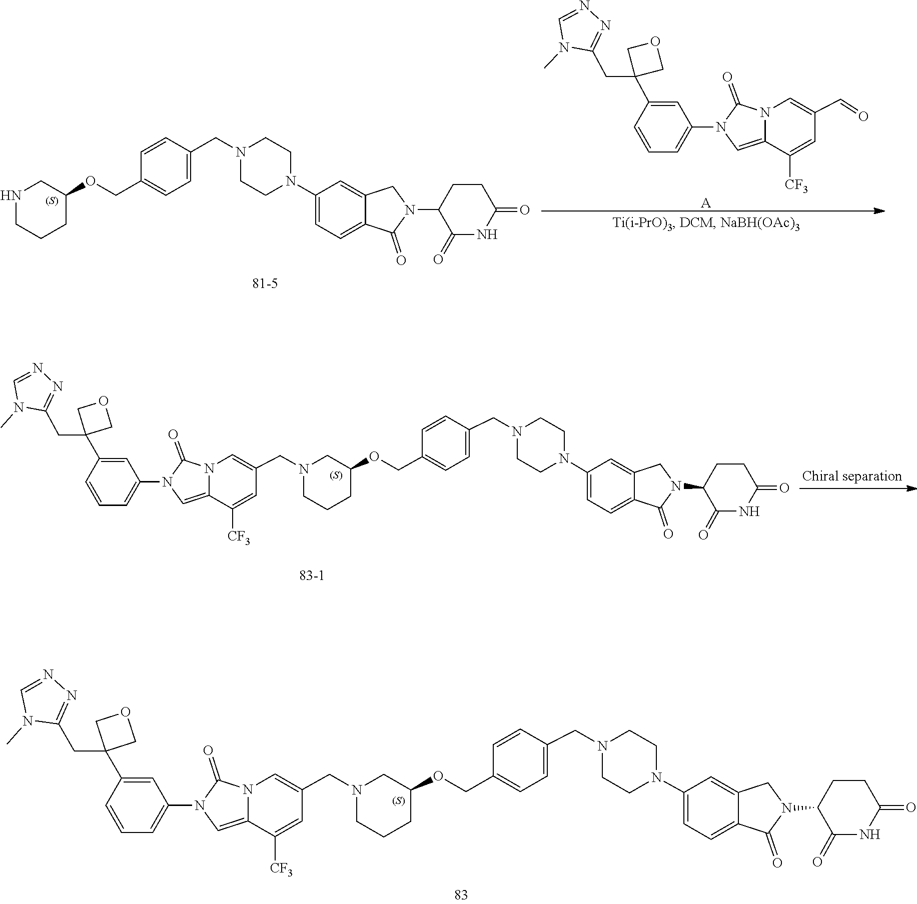

### Synthesis of Compound 83-1.

To a stirred solution of Compound 81-5 (1 g, 1.88 mmol, 1.0 equiv) and Intermediate A (1.3 g, 2.82 mmol, 1.5 equiv) in DCM (10 mL) was added Ti(Oi-Pr)₄ (2.14 g, 7.52 mmol, 4.0 equiv) at room temperature. The resulting mixture was stirred for overnight at room temperature. To the above mixture was added NaBH(OAc)₃ (1.2 g, 5.64 mmol, 3.0 equiv) at room temperature. The resulting mixture was stirred for additional 2 h at room temperature. The reaction was quenched by the addition of 10% AcOH (100 mL) at room temperature. The aqueous layer was extracted with EtOAc (2×60 mL). The aqueous phase was concentrated under reduced pressure. The residue was purified by reversed-phase flash chromatography with the following conditions: column, C18 silica gel; mobile phase, MeCN in water (0.1% FA), 10% to 50% gradient in 30 min; detector, UV 254 nm. This resulted in Compound 83 (220 mg, 12%) as a yellow solid.

### Synthesis of Compound 83.

The Compound 83-1 (220 mg) was purified by Prep-CHIRAL-HPLC with the following conditions Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 60% B to 60% B in 25 min; Wave Length: 220/254 nm; RT1 (min): 6.58; RT2 (min): 13.21; Sample Solvent: DCM:ACN=1:1—HPLC; Injection Volume: 1.2 mL; Number of Runs: 4. The first peak was the product. This resulted in Compound 83 (69 mg, 31%) as a yellow solid.

LC-MS-Compound 83: (ES, m/z): [M+H]⁺ 973

H-NMR-Compound 83: (400 MHz, DMSO-d6, δ ppm): 1.18-1.68 (m, 4H), 1.95-2.05 (m, 4H), 2.34-2.41 (m, 3H), 2.56-2.67 (m, 4H), 2.87-2.97 (m, 5H), 3.27-3.33 (m, 5H), 3.49-3.53 (m, 5H), 4.17-4.22 (d, 1H), 4.30-4.34 (d, 1H), 4.91-4.96 (m, 4H), 5.02-5.07 (m, 1H), 6.88-6.90 (m, 1H), 7.01-7.05 (m, 3H), 7.28-7.41 (m, 7H), 7.51-7.53 (d, 1H), 7.67 (s, 1H), 7.75-7.77 (d, 1H), 8.20 (s, 1H), 10.95 (s, 1H).

### Example 82. Compound 84

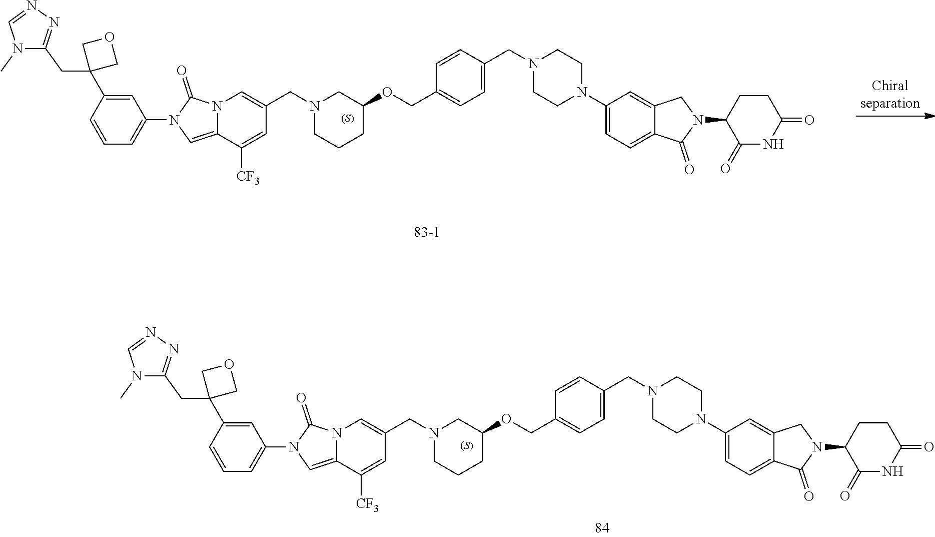

### Synthesis of Compound 84.

The Compound 83-1 (220 mg) was purified by Prep-CHIRAL-HPLC with the following conditions Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 60% B to 60% B in 25 min; Wave Length: 220/254 nm; RT1 (min): 6.58; RT2 (min): 13.21; Sample Solvent: DCM:ACN=1:1—HPLC; Injection Volume: 1.2 mL; Number of Runs: 4. The second peak was the product. This resulted in Compound 84 (65 mg, 31%) as a yellow solid.

LC-MS-Compound 84: (ES, m/z): [M+H]⁺ 973

H-NMR-Compound 84: (400 MHz, DMSO-d6, δ ppm): 1.18-1.68 (m, 4H), 1.95-2.05 (m, 4H), 2.34-2.41 (m, 3H), 2.56-2.67 (m, 4H), 2.87-2.97 (m, 5H), 3.27-3.33 (m, 5H), 3.49-3.53 (m, 5H), 4.17-4.22 (d, 1H), 4.30-4.34 (d, 1H), 4.91-4.96 (m, 4H), 5.02-5.07 (m, 1H), 6.88-6.90 (m, 1H), 7.01-7.05 (m, 3H), 7.28-7.41 (m, 7H), 7.51-7.53 (d, 1H), 7.67 (s, 1H), 7.75-7.77 (d, 1H), 8.20 (s, 1H), 10.95 (s, 1H).

### Example 83. Compound 85

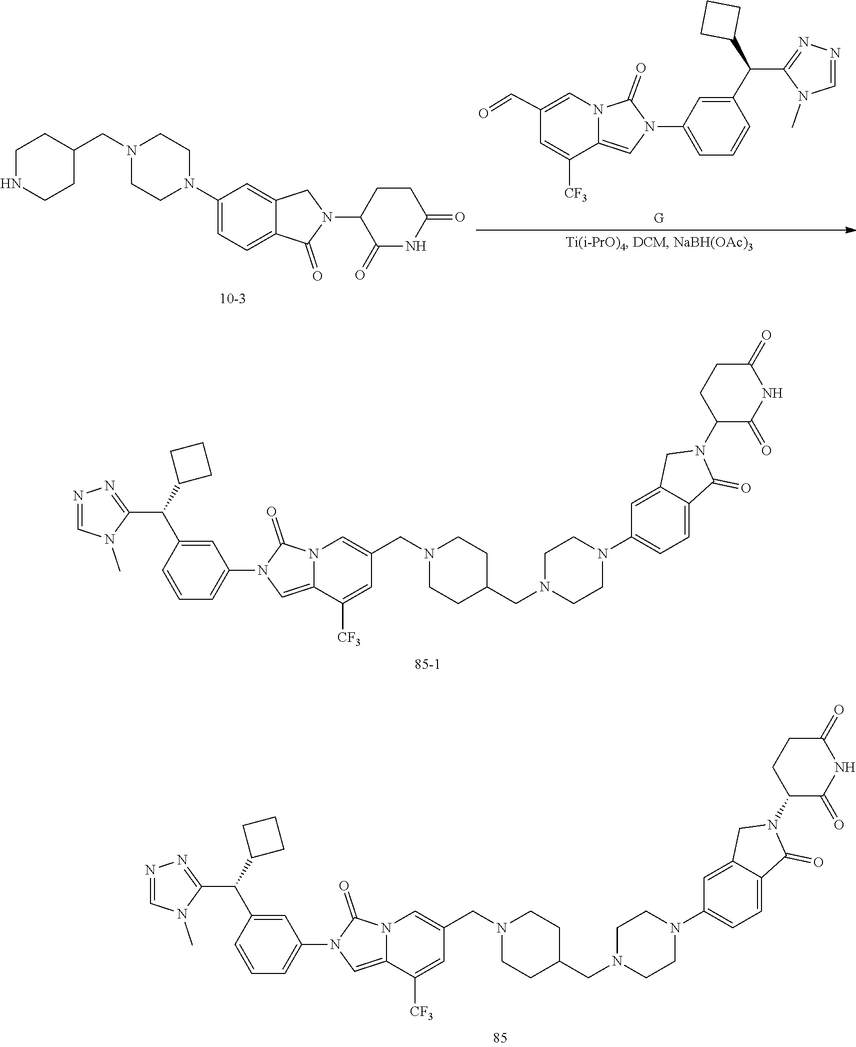 [SMILES: O=C1CCC(N2Cc3cc(N4CCN(CC5CCNCC5)CC4)ccc3C2=O)C(=O)N1 ; Cn1cnnc1[C@@H](c1cccc(-n2cc3c(C(F)(F)F)cc(C=O)cn3c2=O)c1)C1CCC1 ; Cn1cnnc1[C@@H](c1cccc(-n2cc3c(C(F)(F)F)cc(CN4CCC(CN5CCN(c6ccc7c(c6)CN(C6CCC(=O)NC6=O)C7=O)CC5)CC4)cn3c2=O)c1)C1CCC1 ; Cn1cnnc1[C@@H](c1cccc(-n2cc3c(C(F)(F)F)cc(CN4CCC(CN5CCN(c6ccc7c(c6)CN([C@@H]6CCC(=O)NC6=O)C7=O)CC5)CC4)cn3c2=O)c1)C1CCC1]

### Synthesis of Compound 85-1.

To a stirred solution of Compound 10-3 (1 g, 2.35 mmol, 1.0 equiv) and Intermediate G (1.6 g, 3.52 mmol, 1.5 equiv) in DCM (10 mL) was added Ti(Oi-Pr)₄ (2.7 g, 9.40 mmol, 4.0 equiv) at room temperature. The resulting mixture was stirred for overnight at room temperature. To the above mixture was added NaBH(OAc)₃ (1.5 g, 7.05 mmol, 3.0 equiv) at room temperature. The resulting mixture was stirred for additional 2 h at room temperature. The reaction was quenched by the addition of 10% AcOH (100 mL) at room temperature. The aqueous layer was extracted with EtOAc (2×60 mL). The aqueous phase was concentrated under reduced pressure. The residue was purified by reversed-phase flash chromatography with the following conditions: column, C18 silica gel; mobile phase, MeCN in water (0.1% FA), 10% to 50% gradient in 30 min; detector, UV 254 nm. The crude product (350 mg) was purified by Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 10% B to 22% B in 8 min, 22% B; Wave Length: 254; 220 nm; RT1 (min) 7.8) to afford Compound 85-1 (220 mg, 11%) as a yellow solid.

### Synthesis of Compound 85.

The Compound 85-1 (220 mg) was purified by Prep-CHIRAL-HPLC with the following conditions (Prep-HPLC-037): Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 60% B to 60% B in 20 min; Wave Length: 220/254 nm; RT1 (min): 8.35; RT2 (min): 15.02. The first peak was the product. This resulted in Compound 85 (81 mg, 37%) as a yellow solid.

LC-MS-Compound 85: (ES, m/z): [M+H]⁺ 865

H-NMR-Compound 85: (400 MHz, DMSO-d6, δ ppm): 1.11-1.14 (m, 2H), 1.51-1.55 (m, 1H), 1.70-1.80 (m, 7H), 1.94-2.19 (m, 6H), 2.35-2.39 (m, 2H), 2.50-2.60 (m, 1H), 2.84-2.90 (m, 3H), 3.21-3.33 (m, 10H), 3.43 (s, 3H), 4.18-4.35 (m, 3H), 5.02-5.07 (m, 1H), 7.02-7.06 (m, 3H), 7.18-7.20 (d, 1H), 7.32 (s, 1H), 7.42-7.46 (m, 1H), 7.50-7.53 (m, 1H), 7.66-7.73 (m, 3H), 8.33 (s, 1H), 10.95 (s, 1H).

### Example 84. Compound 86

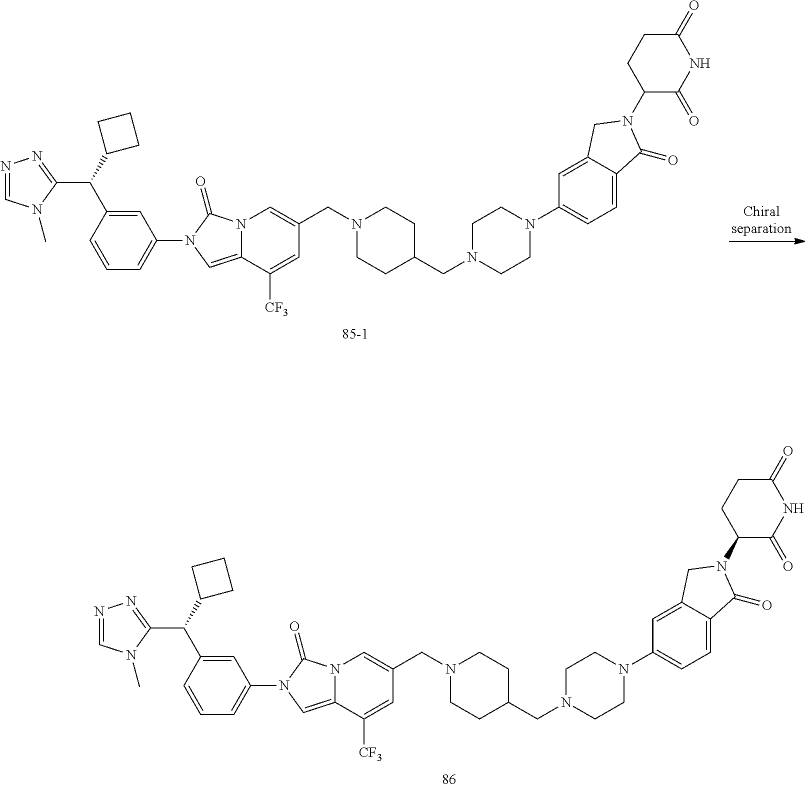 [SMILES: Cn1cnnc1[C@@H](c1cccc(-n2cc3c(C(F)(F)F)cc(CN4CCC(CN5CCN(c6ccc7c(c6)CN(C6CCC(=O)NC6=O)C7=O)CC5)CC4)cn3c2=O)c1)C1CCC1 ; Cn1cnnc1[C@@H](c1cccc(-n2cc3c(C(F)(F)F)cc(CN4CCC(CN5CCN(c6ccc7c(c6)CN([C@H]6CCC(=O)NC6=O)C7=O)CC5)CC4)cn3c2=O)c1)C1CCC1]

### Synthesis of Compound 86.

The Compound 85-1 (220 mg) was purified by Prep-CHIRAL-HPLC with the following conditions (Prep-HPLC-037): Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 60% B to 60% B in 20 min; Wave Length: 220/254 nm; RT1 (min): 8.35; RT2 (min): 15.02. The second peak was the product. This resulted in Compound 86 (70 mg, 31%) as a yellow solid.

LC-MS-Compound 86: (ES, m/z): [M+H]⁺ 865

H-NMR-Compound 86: (400 MHz, DMSO-d6, δ ppm): 1.11-1.14 (m, 2H), 1.51-1.55 (m, 1H), 1.70-1.80 (m, 7H), 1.94-2.19 (m, 6H), 2.35-2.39 (m, 2H), 2.50-2.60 (m, 1H), 2.84-2.90 (m, 3H), 3.21-3.33 (m, 10H), 3.43 (s, 3H), 4.18-4.35 (m, 3H), 5.02-5.07 (m, 1H), 7.02-7.06 (m, 3H), 7.18-7.20 (d, 1H), 7.32 (s, 1H), 7.42-7.46 (m, 1H), 7.50-7.53 (m, 1H), 7.66-7.73 (m, 3H), 8.33 (s, 1H), 10.95 (s, 1H).

### Example 85. Compound 87

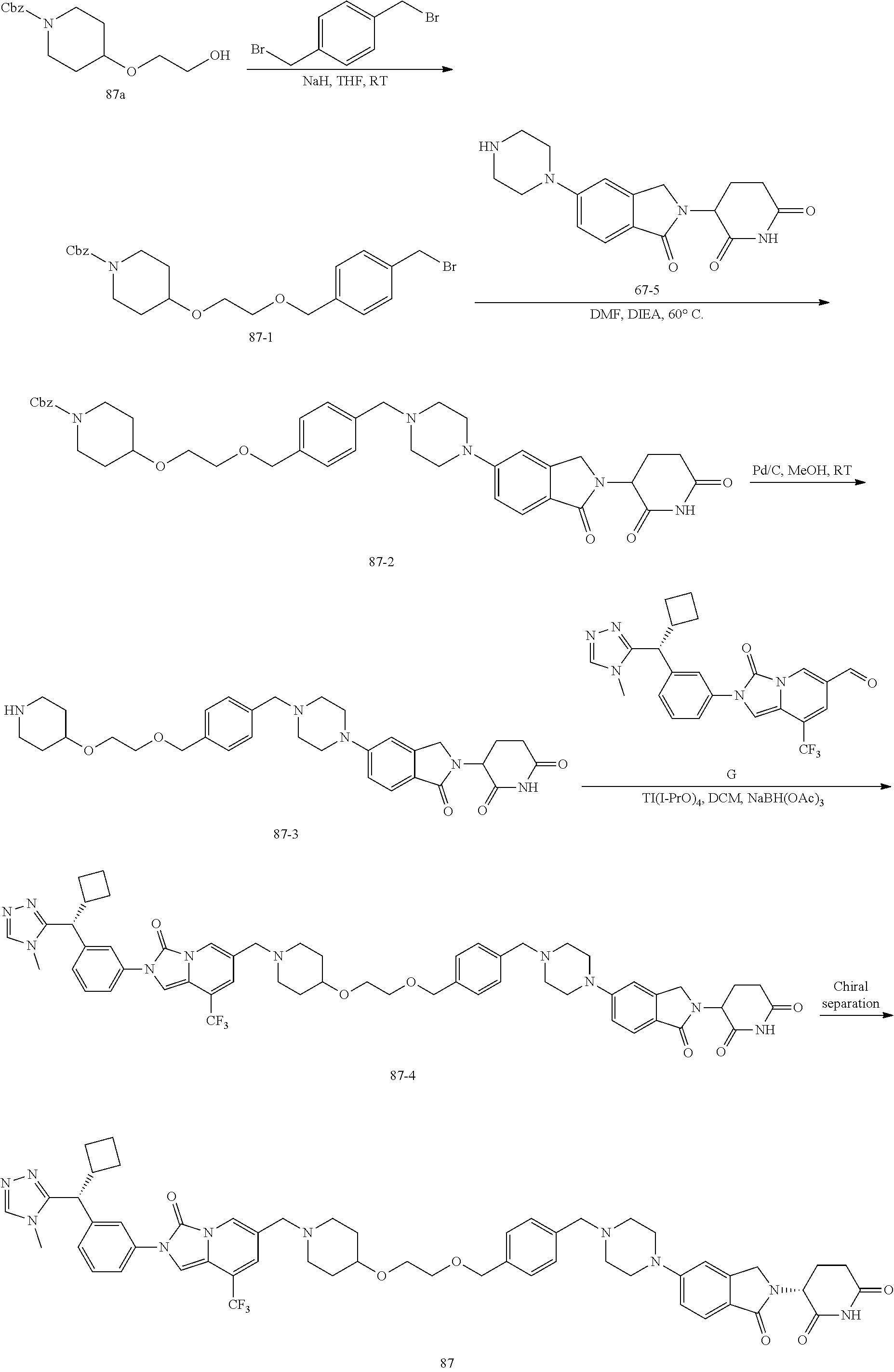

### Synthesis of Compound 87-1.

To a solution of Compound 87a (22.8 g, 81.622 mmol, 1 equiv) in THF (230 mL) was added NaH (4.90 g, 122.433 mmol, 1.5 equiv, 60%) at 0 degrees C. The mixture was stirred for 1 h, 1,4-bis(bromomethyl)benzene (32.32 g, 122.433 mmol, 1.5 equiv) was added and the mixture was allowed to warm to RT and stirred for overnight. The reaction was quenched by the addition of sat. NH₄Cl (aq.) (600 mL). The resulting mixture was extracted with EtOAc (3×200 mL). The combined organic layers were concentrated under reduced pressure. The residue was purified by silica gel column chromatography, eluted with PE/EA (10:1) to afford Compound 87-1 (33 g, 82.19%) as a yellow oil.

### Synthesis of Compound 87-2.

To a stirred solution of Compound 87-1 (1.30 g, 2.814 mmol, 1.2 equiv) and Compound 67-5 (1 g, 2.345 mmol, 1.00 equiv) in DMF (13 mL) was added DIEA (0.61 g, 4.690 mmol, 2 equiv). The resulting mixture was stirred for overnight at 60° C. under nitrogen atmosphere. The reaction was quenched with Water/HOAc=10/1 (40 ml) at room temperature. The resulting mixture was extracted with CH₂Cl₂/MeOH (10:1) (3×20 mL). The combined organic layers were concentrated under reduced pressure. The residue was purified by silica gel column chromatography, eluted with CH₂Cl₂/MeOH (50:1) to afford Compound 87-2 (1.53 g, 91.91%) as a green solid.

### Synthesis of Compound 87-3.

To a solution of Compound 87-2 (1.16 g, 1.634 mmol, 1 equiv) in MeOH (116 mL) was added Ammonium acetate (3.02 g, 39.216 mmol, 24 equiv) and Pd/C (10%, 0.116 g) under nitrogen atmosphere in a 500 mL round-bottom flask. The mixture was hydrogenated at room temperature for 40 min under hydrogen atmosphere using a hydrogen balloon. The resulting mixture was concentrated under vacuum. The residue was purified by reverse flash chromatography with the following conditions: column, C18 silica gel; mobile phase, MeCN in Water (0.1% HOAC), 10% to 40% gradient in 30 min; detector, UV 254 nm. This resulted in Compound 87-3 (300 mg, 31.89%) as a colorless oil.

### Synthesis of Compound 87-4.

To a stirred solution of Compound 87-3 (1 g, 1.737 mmol, 1 equiv) and Intermediate G (0.79 g, 1.737 mmol, 1 equiv) in DCM (20 mL) was added Ti(Oi-Pr)₄ (1.97 g, 6.948 mmol, 4 equiv). The resulting mixture was stirred for overnight at room temperature under nitrogen atmosphere. To the above mixture was added NaBH(OAc)₃ (1.10 g, 5.211 mmol, 3 equiv). The resulting mixture was stirred for additional 6 h at room temperature. The reaction was quenched by the addition of Water/HOAc=10/1 (100 mL). The resulting mixture was extracted with CH₂Cl₂/MeOH=10/1 (3×100 mL). The combined organic layers were concentrated under reduced pressure. The residue was purified by reversed-phase flash chromatography with the following conditions: column, C18 silica gel; mobile phase, MeCN in Water (0.1% FA), 10% to 60% gradient in 10 min; detector, UV 254 nm. This resulted in Compound 87-4350 mg, 19.85%) as a yellow solid.

### Synthesis of Compound 87.

Compound 87-4 (350 mg) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 60% B to 60% B in 15.5 min; Wave Length: 220/254 nm; RT1 (min): 6.61; RT2 (min): 11.74; the first peak was product) to afford Compound 87 (75.9 mg, 21.69%) as a yellow solid.

LCMS-Compound 87: (ES, m/z): [M+H]⁺ 1015

NMR-Compound 87: (400 MHz, DMSO, δ ppm): δ1.42-1.47 (m, 2H), 1.74-1.79 (m, 7H), 1.80-1.95 (m, 1H), 2.07-2.10 (m, 3H), 2.32-2.34 (m, 1H), 2.55-2.59 (m, 1H), 2.67-2.68 (m, 2H), 2.82-2.93 (m, 1H), 3.24-3.28 (m, 7H), 3.32-3.40 (m, 5H), 3.42 (s, 3H), 3.51-3.55 (m, 6H), 4.17-4.34 (m, 3H), 4.48 (s, 2H), 5.02-5.06 (m, 1H), 7.01-7.05 (m, 3H), 7.18-7.20 (d, 1H), 7.27-7.33 (m, 5H), 7.41-7.45 (t, 1H), 7.50-7.52 (d, 1H), 7.67-7.73 (m, 3H), 8.32 (s, 1H), 10.94 (s, 1H).

### Example 86. Compound 88

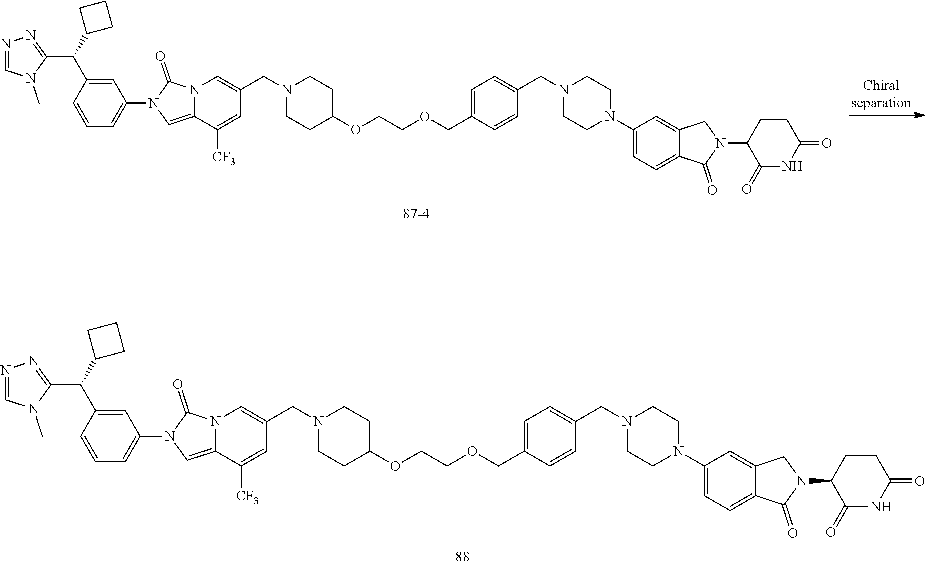 [SMILES: Cn1cnnc1[C@@H](c1cccc(-n2cc3c(C(F)(F)F)cc(CN4CCC(OCCOCc5ccc(CN6CCN(c7ccc8c(c7)CN(C7CCC(=O)NC7=O)C8=O)CC6)cc5)CC4)cn3c2=O)c1)C1CCC1 ; Cn1cnnc1[C@@H](c1cccc(-n2cc3c(C(F)(F)F)cc(CN4CCC(OCCOCc5ccc(CN6CCN(c7ccc8c(c7)CN([C@H]7CCC(=O)NC7=O)C8=O)CC6)cc5)CC4)cn3c2=O)c1)C1CCC1]

### Synthesis of Compound 88.

Compound 87-4 (350 mg) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 60% B to 60% B in 15.5 min; Wave Length: 220/254 nm; RT1 (min): 6.61; RT2 (min): 11.74; the second peak was product) to afford Compound 88 (90.2 mg, 25.77%) as a yellow solid.

LCMS-Compound 88: (ES, m/z): [M+H]⁺ 1015

NMR-Compound 88: (400 MHz, DMSO, δ ppm): 61.42-1.45 (m, 2H), 1.74-1.80 (m, 7H), 1.90-1.95 (m, 1H), 2.07-2.15 (m, 3H), 2.33-2.34 (m, 1H), 2.55-2.67 (m, 3H), 2.82-2.93 (m, 1H), 3.24-3.28 (m, 7H), 3.32-3.40 (m, 5H), 3.42 (s, 3H), 3.51-3.55 (m, 6H), 4.17-4.34 (m, 3H), 4.48 (s, 2H), 5.02-5.06 (m, 1H), 7.01-7.05 (m, 3H), 7.18-7.20 (d, 1H), 7.27-7.33 (m, 5H), 7.41-7.45 (t, 1H), 7.50-7.52 (d, 1H), 7.67-7.70 (m, 2H), 7.73 (s, 1H), 8.32 (s, 1H), 10.94 (s, 1H).

### Example 87. Compound 89

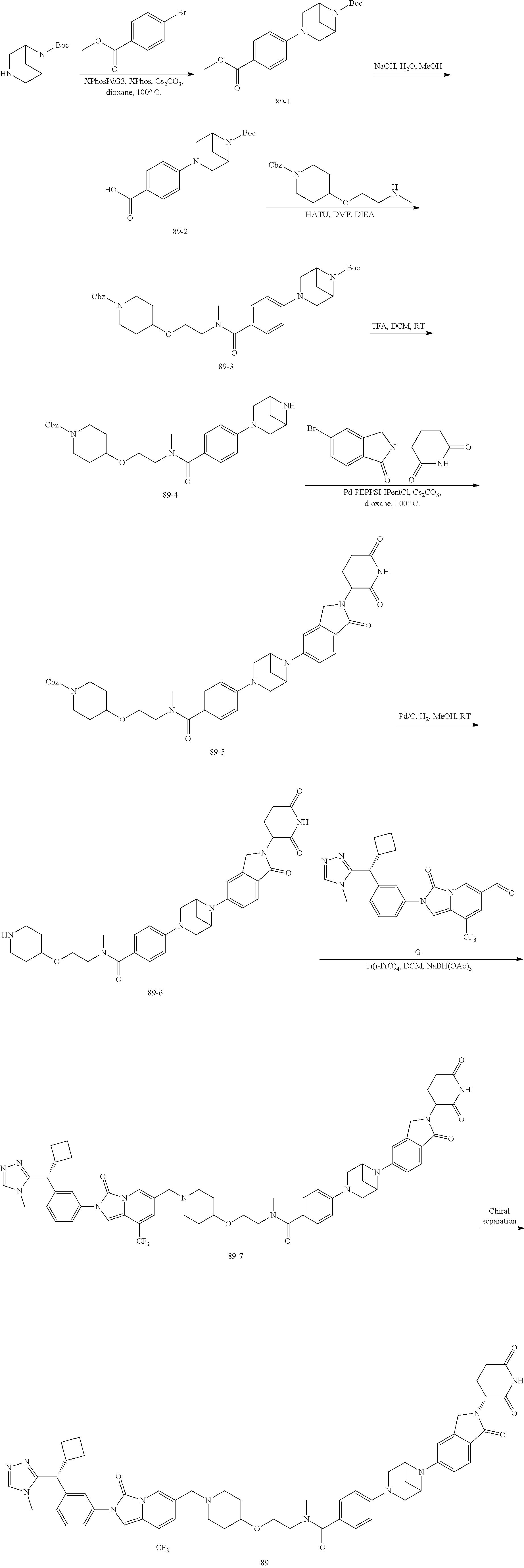

### Synthesis of Compound 89-1.

To a stirred solution of tert-butyl 3,6-diazabicyclo[3.1.1]heptane-6-carboxylate (20 g, 100.875 mmol, 1 equiv) and methyl 4-bromobenzoate (43.39 g, 201.750 mmol, 2 equiv) in dioxane (200 mL) were added XPhos (9.62 g, 20.175 mmol, 0.2 equiv) and Cs₂CO₃ (98.60 g, 302.625 mmol, 3 equiv)) and XPhos Pd G3 (8.54 g, 10.088 mmol, 0.1 equiv) in portions at 100° C. under nitrogen atmosphere. The resulting mixture was stirred for overnight at 100° C. under nitrogen atmosphere. The reaction was quenched with Water at room temperature. The aqueous layer was extracted with CH₂Cl₂ (2×200 mL). The residue was purified by silica gel column chromatography, eluted with CH₂Cl₂/MeOH (10:1) to afford Compound 89-1 (33 g, 49.21%) as a yellow solid.

### Synthesis of Compound 89-2.

A solution of tert-butyl 3-[4-(methoxycarbonyl)phenyl]-3,6-diazabicyclo[3.1.1]heptane-6-carboxylate Compound 89-1 (34.1 g, 102.587 mmol, 1 equiv) in MeOH (10 mL) was treated with NaOH (16.41 g, 410.348 mmol, 4 equiv) and H₂O (440 mL) for overnight at room temperature under nitrogen atmosphere. The mixture was acidified to pH 5 with HCl (aq.). The precipitated solids were collected by filtration and washed with H₂O (2×100 mL). This resulted in Compound 89-2 (28.4 g, 79.13%) as a white solid.

### Synthesis of Compound 89-3.

A solution of Compound 89-2 (6.1 g, 19.160 mmol, 1 equiv) in DMF (61 mL) was treated with HATU (10.93 g, 28.740 mmol, 1.5 equiv) and DIEA (9.91 g, 76.640 mmol, 4 equiv) for 1 h at 0° C. under nitrogen atmosphere followed by the addition of benzyl 4-[2-(methylamino)ethoxy]piperidine-1-carboxylate (5.60 g, 19.160 mmol, 1 equiv) at room temperature. The resulting mixture was stirred for overnight at room temperature under nitrogen atmosphere. The product was precipitated by the addition of H₂O (150 mL). This resulted in Compound 89-3 (11.5 g, 96.20%) as a white solid.

### Synthesis of Compound 89-4.

A solution of Compound 89-3 (11.5 g, 19.402 mmol, 1 equiv) in DCM (86 mL) was treated with TFA (28 mL, 376.966 mmol, 19.43 equiv) for 1 h at room temperature under nitrogen atmosphere. The mixture was basified to pH 7 with saturated NaHCO₃ (aq.). The aqueous layer was extracted with CH₂Cl₂/MeOH (10:1) (3×100 mL). The resulting mixture was concentrated under vacuum. This resulted in Compound 89-4 (3.77 g, 38.66%) as a yellow oil.

### Synthesis of Compound 89-5.

To a stirred mixture of Compound 89-4 (2 g, 4.060 mmol, 1 equiv) and 3-(5-bromo-1-oxo-3H-isoindol-2-yl)piperidine-2,6-dione (1.57 g, 4.872 mmol, 1.2 equiv) in dioxane (60 mL) were added Cs₂CO₃ (3.97 g, 12.180 mmol, 3 equiv) and Pd-PEPPSI-IPentCl 2-methylpyridine (o-picoline) (0.34 g, 0.406 mmol, 0.1 equiv) under nitrogen atmosphere. The resulting mixture was stirred for overnight at 100° C. under nitrogen atmosphere. The resulting mixture was diluted with water/HOAc=10/1 (240 mL). The resulting mixture was extracted with CH2Cl2/MeOH=10/1 (3×200 mL). The combined organic layers were concentrated under reduced pressure. The residue was purified by silica gel column chromatography, eluted with CH₂Cl2/MeOH (7:1) to afford Compound 89-5 (2.4 g, 80.44%) as a light yellow solid.

### Synthesis of Compound 89-6.

To a solution of Compound 89-5 (2.4 g, 3.266 mmol, 1 equiv) in MeOH (50 mL) was added Pd/C (400 mg, 10%) under nitrogen atmosphere in a 250 mL round-bottom flask. The mixture was hydrogenated at room temperature for 2 h under hydrogen atmosphere using a hydrogen balloon, filtered through a Celite pad and concentrated under reduced pressure. This resulted in Compound 89-6 (1.8 g, 91.75%) as a light yellow solid.

### Synthesis of Compound 89-7.

To a stirred solution of Compound 89-6 (900 mg, 1.498 mmol, 1 equiv) and Intermediate G (682.34 mg, 1.498 mmol, 1 equiv) in DCM (18 mL) was added Ti(Oi-Pr)₄ (1703.27 mg, 5.992 mmol, 4 equiv). The resulting mixture was stirred for 1 h at room temperature. To the above mixture was added NaBH(OAc)₃ (952.59 mg, 4.494 mmol, 3 equiv). The resulting mixture was stirred for additional 6 h at room temperature. The reaction was quenched by the addition of Water/HOAc=10/1 (100 mL). The resulting mixture was extracted with CH₂Cl₂/MeOH (3×100 mL). The combined organic layers were concentrated under reduced pressure. The residue was purified by reverse flash chromatography with the following conditions: column, C18 silica gel; mobile phase, MeCN in Water (0.1% FA), 5% to 55% gradient in 30 min; detector, UV 254 nm. This resulted in Compound 89-7 (840 mg, 53.90%) as a yellow solid.

### Synthesis of Compound 89.

Compound 89-7 (400 mg) was purified by Prep-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 90% B to 90% B in 22 min; Wave Length: 220/254 nm; RT1 (min): 5.74; RT2 (min): 14.90; the first peak was product) to afford Compound 89-0 (151.6 mg, 37.90%) as a yellow solid.

LCMS-Compound 89: (ES, m/z): [M+H]⁺ 1040

NMR-Compound 89: (400 MHz, DMSO, δ ppm): δ1.35-1.44 (m, 2H), 1.70-1.94 (m, 1OH), 2.07-2.14 (m, 3H), 2.23-2.34 (m, 1H), 2.51-2.66 (m, 3H), 2.72-2.79 (m, 1H), 2.81-2.94 (m, 1H), 2.97 (s, 3H), 3.15-3.29 (m, 5H), 3.42-3.53 (m, 7H), 3.76-3.80 (d, 2H), 4.14-4.18 (m, 1H), 4.24-4.37 (m, 2H), 4.59-4.60 (m, 2H), 4.96-5.03 (m, 1H), 6.58-6.60 (m, 2H), 6.72-6.80 (m, 2H), 7.01 (s, 1H), 7.18-7.31 (m, 3H), 7.42-7.48 (m, 2H), 7.65-7.74 (m, 3H), 8.33 (s, 1H), 10.91 (s, 1H).

### Example 88. Compound 90

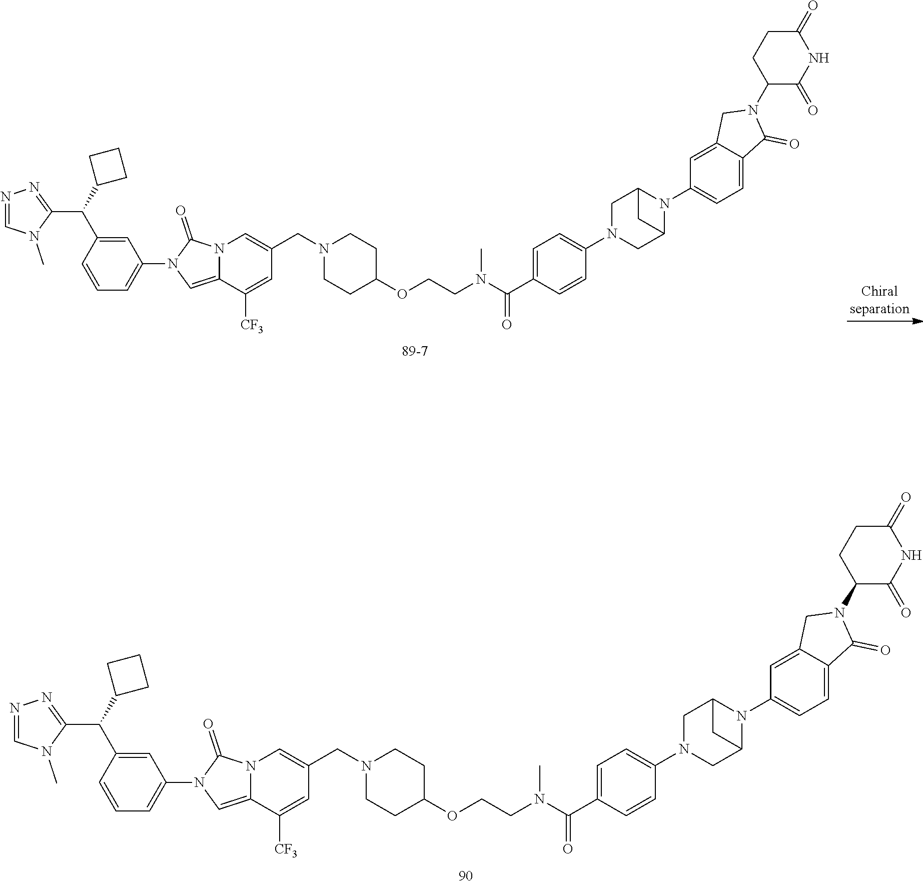 [SMILES: CN(CCOC1CCN(Cc2cc(C(F)(F)F)c3cn(-c4cccc([C@H](c5nncn5C)C5CCC5)c4)c(=O)n3c2)CC1)C(=O)c1ccc(N2CC3CC(C2)N3c2ccc3c(c2)CN(C2CCC(=O)NC2=O)C3=O)cc1 ; CN(CCOC1CCN(Cc2cc(C(F)(F)F)c3cn(-c4cccc([C@H](c5nncn5C)C5CCC5)c4)c(=O)n3c2)CC1)C(=O)c1ccc(N2CC3CC(C2)N3c2ccc3c(c2)CN([C@H]2CCC(=O)NC2=O)C3=O)cc1]

### Synthesis of Compound 90.

Compound 89-7 (400 mg) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 90% B to 90% B in 22 min; Wave Length: 220/254 nm; RT1 (min): 5.74; RT2 (min): 14.90; Sample Solvent: DCM:ACN=1:1; the second peak was product) to afford Compound 90 (91.9 mg, 22.98%) as a yellow solid.

LCMS-Compound 89: (ES, m/z): [M+H]⁺ 1040

NMR-Compound 89: (400 MHz, DMSO, δ ppm): 61.35-1.44 (m, 2H), 1.70-1.94 (m, 10H), 2.11-2.14 (m, 3H), 2.23-2.34 (m, 1H), 2.51-2.66 (m, 3H), 2.72-2.79 (m, 1H), 2.81-2.97 (m, 4H), 3.15-3.29 (m, 4H), 3.32-3.35 (m, 1H), 3.42-3.53 (m, 7H), 3.76-3.80 (d, 2H), 4.14-4.37 (m, 3H), 4.59-4.60 (m, 2H), 4.98-5.03 (m, 1H), 6.58-6.60 (m, 2H), 6.72-6.80 (m, 2H), 7.01 (s, 1H), 7.18-7.31 (m, 3H), 7.42-7.48 (m, 2H), 7.65-7.74 (m, 3H), 8.32 (s, 1H), 10.91 (s, 1H).

### Example 89. Compound 91

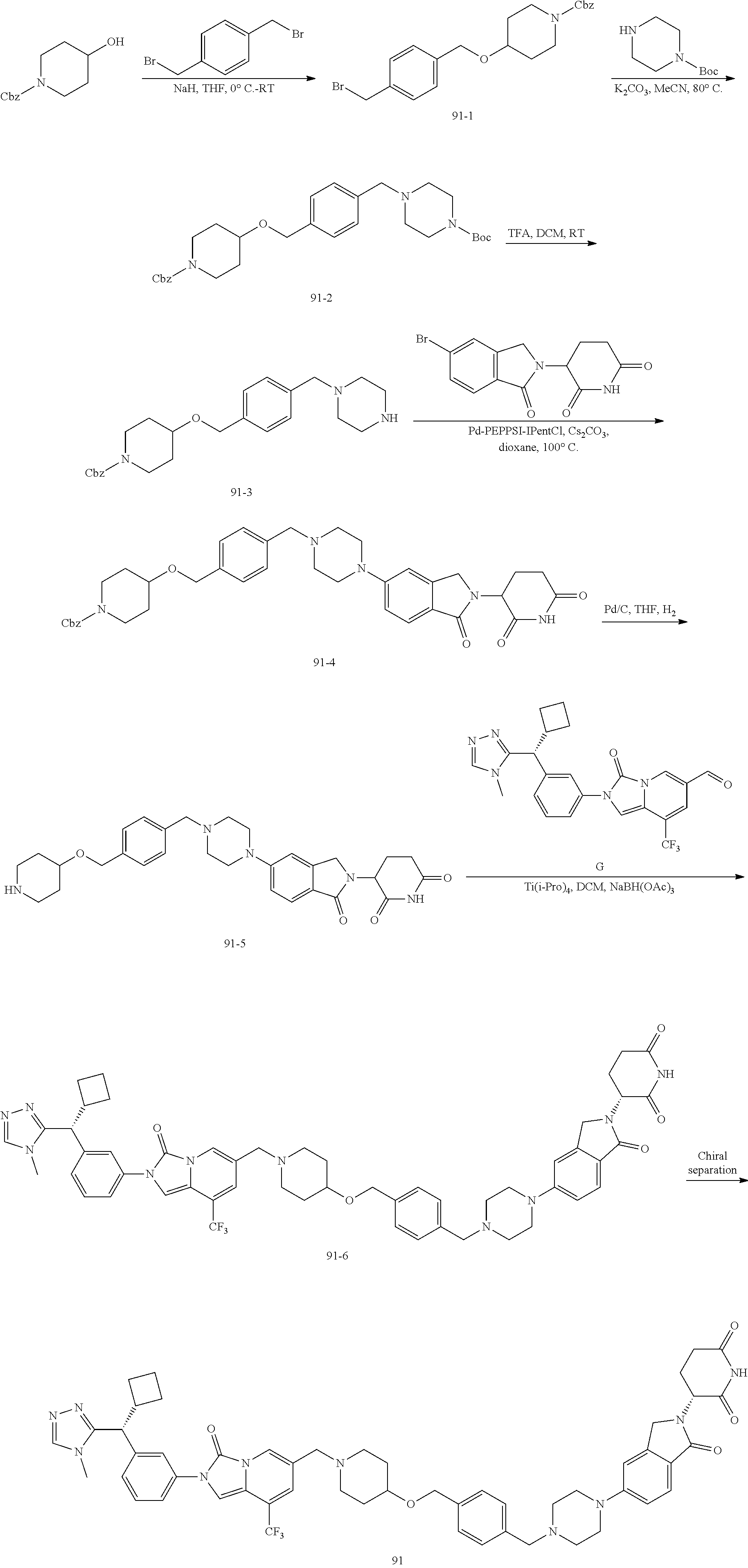

### Synthesis of Compound 91-1.

To a solution of benzyl 4-hydroxypiperidine-1-carboxylate (20.0 g, 85.00 mmol, 1.0 equiv) in THF (200 mL) was added sodium hydride (4.1 g, 10.25 mmol, 1.2 equiv, 60% in oil) at 0° C. The mixture was stirred for 30 min. To the above 1,4-bis(bromomethyl)benzene (33.6 g, 127.51 mmol, 1.5 equiv) was added and the mixture was allowed to warm to RT and stirred for overnight. The reaction was quenched with sat. NH₄Cl (aq.) (600 mL) at room temperature. The aqueous layer was extracted with EtOAc (2×300 mL). The resulting mixture was concentrated under reduced pressure. The residue was purified by silica gel column chromatography, eluted with PE/EA=1:1 to afford Compound 91-1 (17.0 g, 43%) as a green oil.

### Synthesis of Compound 91-2.

To a stirred solution of Compound 91-1 (17 g, 40.64 mmol, 1.0 equiv) and tert-butyl piperazine-1-carboxylate (11.3 g, 60.96 mmol, 1.5 equiv) in MeCN (170 mL) was added K₂CO₃ (16.8 g, 121.91 mmol, 3 equiv) at room temperature under nitrogen atmosphere. The resulting mixture was stirred for overnight at 80° C. under nitrogen atmosphere. The reaction was quenched by the addition of sat. NH₄Cl (aq.) (500 mL) at room temperature. The aqueous layer was extracted with CH₂Cl₂ (3×200 mL). The resulting mixture was concentrated under reduced pressure. The residue was purified by silica gel column chromatography, eluted with PE/EA=2:1 to afford Compound 91-2 (13.0 g, 55%) as a yellow green oil.

### Synthesis of Compound 91-3.

To a stirred solution of Compound 91-2 (13.0 g, 24.83 mmol, 1.0 equiv) in DCM (104 mL) was added TFA (26 mL) at room temperature under nitrogen atmosphere. The resulting mixture was stirred for overnight at room temperature under nitrogen atmosphere. The resulting mixture was concentrated under reduced pressure. The mixture was neutralized to pH 7 with saturated Na₂CO₃ (aq.). The aqueous layer was extracted with CH₂Cl₂ (3×300 mL). The resulting mixture was concentrated under reduced pressure. The crude product was used in the next step directly without further purification. This resulted in Compound 91-3 (10.0 g, 85%) as a brown oil.

### Synthesis of Compound 91-4.

To a stirred solution of Compound 91-3 (10.0 g, 23.61 mmol, 1.0 equiv) and 3-(5-bromo-1-oxo-3H-isoindol-2-yl)piperidine-22,6-dione (9.2 g, 28.33 mmol, 1.2 equiv) in dioxane (100 mL) were added Cs₂CO₃ (23.1 g, 70.83 mmol, 3.0 equiv) and Pd PEPPSI IPentCl (2.0 g, 2.36 mmol, 0.1 equiv) at room temperature under nitrogen atmosphere. The resulting mixture was stirred for overnight at 100° C. under nitrogen atmosphere. The reaction was quenched by the addition of Water/HOAc=10/1 (1000 mL) at room temperature. The aqueous layer was extracted with CH₂Cl₂ (3×400 mL). The resulting mixture was concentrated under reduced pressure. The residue was purified by silica gel column chromatography, eluted with CH₂Cl₂/MeOH=20:1 to afford Compound 91-4 (6.3 g, 36%) as a brown oil.

### Synthesis of Compound 91-5.

To a solution of Compound 91-4 (3.0 g, 4.51 mmol, 1 equiv) in 150 mL THF was added Pd/C (10%, 0.9 g) under nitrogen atmosphere in a 500 mL round-bottom flask. The mixture was hydrogenated at room temperature for 6 h under hydrogen atmosphere using a hydrogen balloon, filtered through a Celite pad and concentrated under reduced pressure. The resulting mixture was filtered, the filter cake was washed with CH₂Cl₂ (5×100 mL). The filtrate was concentrated under reduced pressure. The crude product was used in the next step directly without further purification. This resulted in Compound 91-5 (1.6 g, 60%) as a grey solid.

### Synthesis of Compound 91-6.

To a stirred solution of Compound 91-5 (780 mg, 1.47 mmol, 1.0 equiv) and Intermediate G (668 mg, 1.47 mmol, 1.0 equiv) in DCM (8 mL) was added Ti(Oi-Pr)₄ (1667 mg, 5.87 mmol, 4.0 equiv) at room temperature under nitrogen atmosphere. The resulting mixture was stirred for overnight at room temperature under nitrogen atmosphere. To the above mixture was added NaBH(OAc)₃ (933 mg, 4.40 mmol, 3.0 equiv) at room temperature. The resulting mixture was stirred for additional 2 h at room temperature. The reaction was quenched by the addition of Water/HOAc=10/1 (100 mL) at room temperature. The aqueous layer was extracted with EtOAc (2×100 mL). The resulting mixture was filtered, the filter cake was washed with CH₂Cl₂/MeOH=5:1 (3×30 mL). The filtrate was concentrated under reduced pressure. The residue was purified by reversed-phase flash chromatography with the following conditions: column, C18 silica gel; mobile phase, MeCN in water (0.1% FA), 10% to 50% gradient in 30 min; detector, UV 254 nm. This resulted in Compound 91-6 (420 mg, 29%) as a yellow solid.

### Synthesis of Compound 91.

The Compound 91-6 (420 mg, 0.43 mmol, 1.0 equiv) was purified by Prep-Chiral-HPLC with the following conditions (Column: CHIRALPAK IH, 2*25 cm, 5 μm, Mobile Phase A: MTBE (0.1% TEA), Mobile Phase B: DCM:ACN=1:1; Flow rate: 20 mL/min; Gradient: 60% B to 60% B in 17 min; Wave Length: 220/254 nm; RT1 (min): 6.76; RT2 (min): 12.23; the first peak was product) to afford Compound 91 (152 mg, 35%) as a yellow solid.

LCMS-Compound 91: (ES, m/z): [M+H]⁺ 971

NMR-Compound 91: (400 MHz, DMSO-d6, δ ppm): 1.51-1.54 (d, 2H), 1.79-1.86 (m, 8H), 2.08-2.18 (m, 3H), 2.34-2.50 (m, 1H), 2.51-2.70 (m, 8H), 2.80-2.99 (m, 1H), 3.23-3.34 (m, 5H), 3.40-3.52 (m, 7H), 4.17-4.35 (m, 3H), 4.489 (s, 2H), 5.03-5.07 (m, 1H), 7.02-7.06 (m, 3H), 7.18-7.21 (m, 1H), 7.30-7.44 (m, 5H), 7.46-7.53 (m, 2H), 7.67-7.74 (m, 3H), 8.33 (s, 1H), 10.99 (s, 1H).

### Example 90. Compound 92

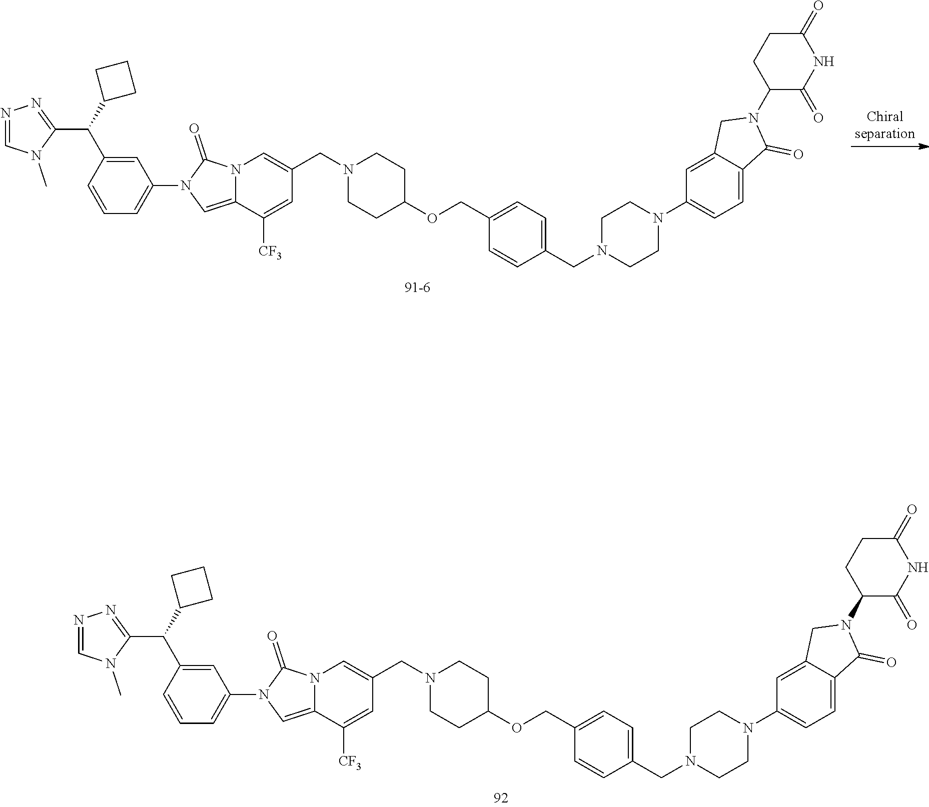 [SMILES: Cn1cnnc1[C@@H](c1cccc(-n2cc3c(C(F)(F)F)cc(CN4CCC(OCc5ccc(CN6CCN(c7ccc8c(c7)CN(C7CCC(=O)NC7=O)C8=O)CC6)cc5)CC4)cn3c2=O)c1)C1CCC1 ; Cn1cnnc1[C@@H](c1cccc(-n2cc3c(C(F)(F)F)cc(CN4CCC(OCc5ccc(CN6CCN(c7ccc8c(c7)CN([C@H]7CCC(=O)NC7=O)C8=O)CC6)cc5)CC4)cn3c2=O)c1)C1CCC1]

### Synthesis of Compound 92.

The Compound 91-6 (420 mg, 0.43 mmol, 1.0 equiv) was purified by Prep-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 60% B to 60% B in 17 min; Wave Length: 220/254 nm; RT1 (min): 6.76; RT2 (min): 12.23; the second peak was product) to afford Compound 92 (157 mg, 37%) as a yellow solid.

LCMS-Compound 92: (ES, m/z): [M+H]⁺ 971

NMR-Compound 92: (400 MHz, DMSO-d6, δ ppm): 1.51-1.54 (m, 2H), 1.75-1.97 (m, 8H), 2.08-2.19 (m, 3H), 2.32-2.38 (m, 1H), 2.51-2.70 (m, 8H), 2.85-2.99 (m, 1H), 3.29-3.34 (m, 5H), 3.43-3.52 (m, 7H), 4.17-4.35 (m, 3H), 4.49 (s, 2H), 5.03-5.07 (m, 1H), 7.02-7.20 (m, 4H), 7.28-7.32 (m, 5H), 7.42-7.53 (m, 2H), 7.67-7.74 (m, 3H), 8.33 (s, 1H), 10.99 (s, 1H).

### Example 91. Compound 93

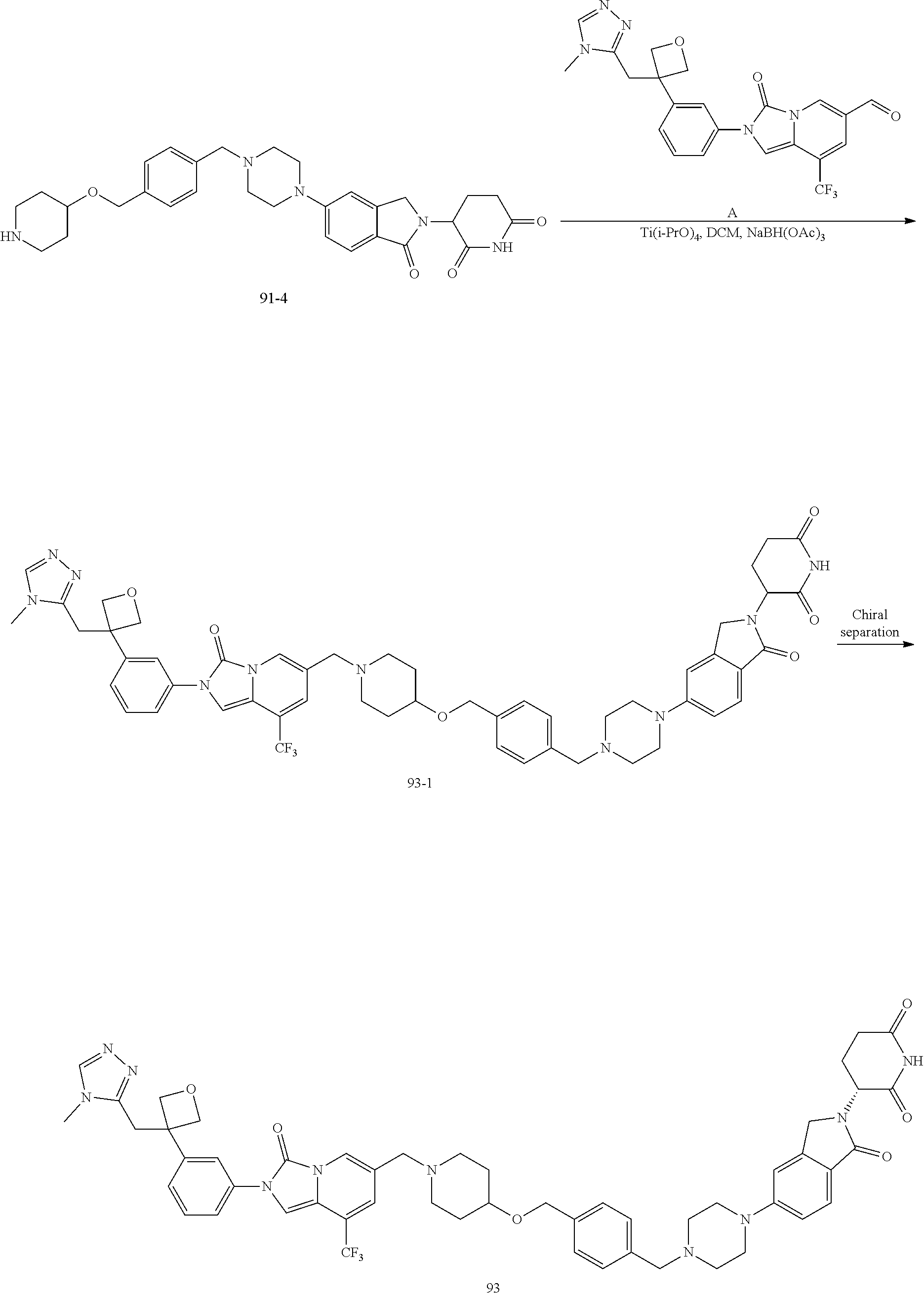

### Synthesis of Compound 93-1.

To a stirred solution of Compound 91-4 (780 mg, 1.47 mmol, 1.0 equiv) and Intermediate A (671 mg, 1.47 mmol, 1.0 equiv) in DCM (8 mL) was added Ti(Oi-Pr)₄ (1.7 g, 5.87 mmol, 4.0 equiv) at room temperature under nitrogen atmosphere. The resulting mixture was stirred for overnight at room temperature. To the above mixture was added NaBH(OAc)₃ (933 mg, 4.40 mmol, 3.0 equiv) at room temperature. The resulting mixture was stirred for additional 2 h at room temperature. The reaction was quenched by the addition of Water/HOAc=10/1 (100 mL) at room temperature. The aqueous layer was extracted with EtOAc (2×100 mL). The resulting mixture was filtered, the filter cake was washed with water (2×20 mL). The filtrate was concentrated under reduced pressure. The residue was purified by reverse flash chromatography with the following conditions: column, C18 silica gel; mobile phase, MeCN in water (0.1% FA), 5% to 50% gradient in 30 min; detector, UV 254 nm. This resulted in Compound 93-1 (400 mg, 27%) as a yellow solid.

### Synthesis of Compound 93.

The Compound 93-1 (400 mg, 0.41 mmol, 1.0 equiv) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 ml/min; Gradient: 60% B to 60% B in 30 min; Wave Length: 220/254 nm; RT1 (min): 12.71; RT2 (min); 23.35; the first peak was product) to afford Compound 93 (143 mg, 35%) as a yellow solid.

LCMS-Compound 93: (ES, m/z): [M+H]⁺ 973

NMR-Compound 93: (400 MHz, DMSO-d6, δ ppm): 1.51-1.54 (m, 2H), 1.86-1.96 (m, 3H), 2.13-2.19 (m, 2H), 2.33-2.38 (m, 1H), 2.49-2.51 (m, 3H), 2.56-2.70 (m, 3H), 2.87-2.96 (m, 4H), 3.29-3.33 (m, 5H), 3.33-3.42 (m, 2H), 3.51-3.53 (m, 5H), 4.17-4.22 (d, 1H), 4.30-4.34 (d, 1H), 4.48 (s, 2H), 4.90-4.96 (m, 4H), 5.02-5.07 (m, 1H), 6.88-6.90 (m, 1H), 7.02-7.05 (m, 3H), 7.29-7.32 (m, 5H), 7.50-7.52 (m, 2H), 7.67 (s, 1H), 7.74-7.76 (d, 2H), 8.20 (s, 1H), 10.95 (s, 1H).

### Example 92. Compound 94

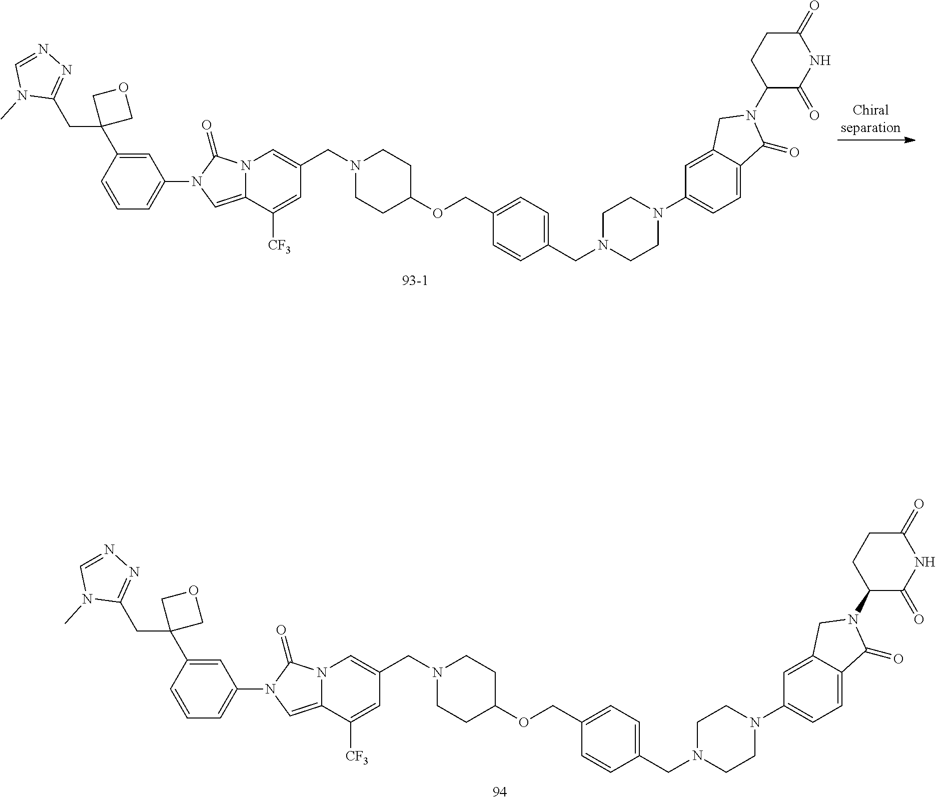

### Synthesis of Compound 94.

The Compound 93-1 (400 mg, 0.41 mmol, 1.0 equiv) was purified by Prep-Chiral-HPLC with the following conditions (Column: CHIRALPAK IH, 2*25 cm, 5 μm, Mobile Phase A: MTBE (0.1% TEA), Mobile Phase B: DCM:ACN=1:1; Flow rate: 20 mL/min; Gradient: 60% B to 60% B in 30 min; Wave Length: 220/254 nm; RT1 (min): 12.71; RT2 (min): 23.35; the second peak was product) to afford Compound 94 (117 mg, 29%) as a yellow solid.

LCMS-Compound 94: (ES, m/z): [M+H]⁺ 973

NMR-Compound 94: (400 MHz, DMSO-d6, δ ppm): 1.51-1.54 (m, 2H), 1.86-1.95 (m, 3H), 2.17 (s, 2H), 2.33-2.35 (m, 1H), 2.49-2.51 (m, 3H), 2.56-2.70 (m, 3H), 2.89-2.99 (m, 4H), 3.29-3.32 (m, 5H), 3.35-3.72 (m, 2H), 3.42-3.53 (m, 5H), 4.17-4.22 (d, 1H), 4.30-4.34 (d, 1H), 4.48 (s, 2H), 4.90-4.96 (m, 4H), 5.02-5.07 (d, 1H), 6.88-6.90 (d, 1H), 7.02-7.05 (m, 3H), 7.29-7.40 (m, 5H), 7.40-7.42 (t, 2H), 7.50-7.50 (d 1H), 7.67 (s, 1H), 7.74-7.76 (d, 1H), 8.20 (s, 1H), 10.95 (s, 1H).

### Example 93. Compound 95

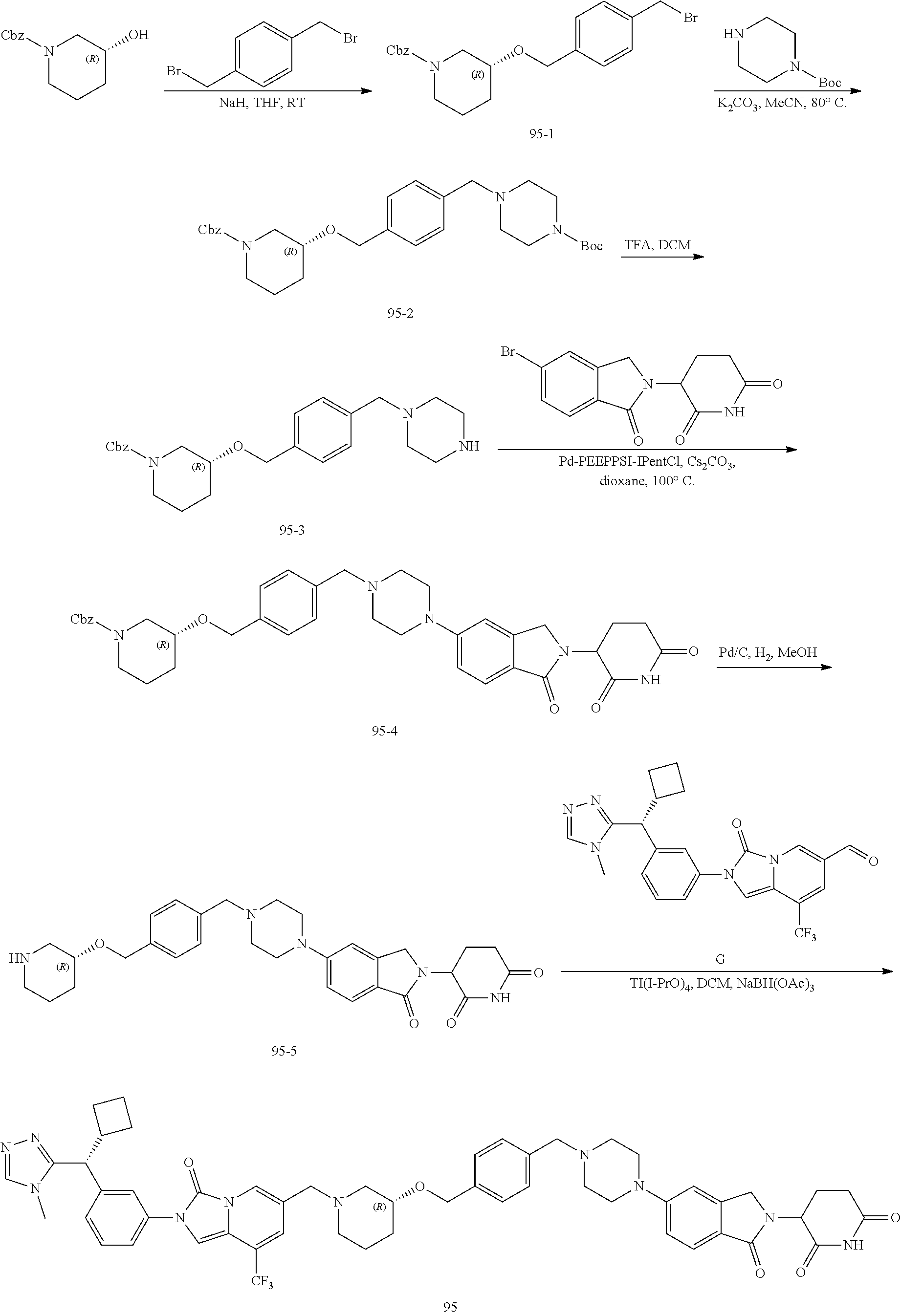

### Synthesis of Compound 95-1.

A mixture of benzyl (3R)-3-hydroxypiperidine-1-carboxylate (10 g, 42.5 mmol, 1 equiv) and NaH (2.55 g, 63.753 mmol, 1.5 equiv, 60% in oil) in THF (100 mL) was stirred for 1 h at 0° C. under nitrogen atmosphere. To the above mixture was added 1,4-bis(bromomethyl)benzene (16.83 g, 63.753 mmol, 1.5 equiv) at 0° C. The resulting mixture was stirred for additional overnight at room temperature. The mixture was acidified to pH 7 with saturated NH₄Cl (aq.). The aqueous layer was extracted with EtOAc (3×20 mL). The resulting mixture was concentrated under reduced pressure. The residue was purified by silica gel column chromatography, eluted with PE/EA (10:1) to afford b Compound 95-1 (10 g, 52.31%) as a colorless oil.

### Synthesis of Compound 95-2.

To a stirred mixture of Compound 95-1 (11 g, 26.295 mmol, 1 equiv) and tert-butyl piperazine-1-carboxylate (7.35 g, 39.443 mmol, 1.5 equiv) in MeCN (110 mL) was added K₂CO₃ (10.90 g, 78.885 mmol, 3 equiv) at room temperature under nitrogen atmosphere. The resulting mixture was stirred for overnight at 80° C. under nitrogen atmosphere. The reaction was quenched by the addition of water (200 mL) at room temperature. The aqueous layer was extracted with EtOAc (3×150 mL). The resulting mixture was concentrated under reduced pressure. The residue was purified by silica gel column chromatography, eluted with PE/EA (10:1) to afford Compound 95-2 (13 g, 86.86%) as a white solid.

### Synthesis of Compound 95-3.

A mixture of Compound 95-2 (13 g, 24.825 m mol, 1 equiv) and TFA (32.5 mL) in DCM (97.5 mL) was stirred for 2 h at room temperature under nitrogen atmosphere. The resulting mixture was concentrated under reduced pressure. The residue was purified by silica gel column chromatography, eluted with CH₂Cl₂/MeOH (20:1) to afford Compound 95-3 (10 g, 90.35%) as a yellow solid.

### Synthesis of Compound 95-4.

To a stirred mixture of benzyl Compound 95-3 (5 g, 11.805 mmol, 1.00 equiv) and 3-(5-bromo-1-oxo-3H-isoindol-2-yl)piperidine-2,6-dione (4.58 g, 14.166 mmol, 1.20 equiv) in were added Cs₂CO₃ (11.54 g, 35.415 mmol, 3 equiv) and Pd-PEPPSI-IPentCl 2-methylpyridine (o-picoline) (0.99 g, 1.181 mmol, 0.1 equiv) under argon atmosphere. The resulting mixture was stirred for overnight at 100° C. under argon atmosphere. The reaction was quenched by the addition of Water/Ice=10/1 (300 mL) at room temperature. The resulting mixture was extracted with CH₂Cl₂ (2×400 mL). The combined organic layers were concentrated under reduced pressure. The residue was purified by silica gel column chromatography, eluted with CH₂Cl₂/MeOH (80:1) to afford Compound 95-4 (2.5 g, 31.81%) as a grey solid.

### Synthesis of Compound 95-5.

To a solution of Compound 95-4 (2.5 g, 3.755 mmol, 1 equiv) in EtOAc (100 mL) was added Pd/C (400 mg, 10%) under nitrogen atmosphere in a 250 mL round-bottom flask. The mixture was hydrogenated at room temperature for 2 h under hydrogen atmosphere using a hydrogen balloon, filtered through a Celite pad and concentrated under reduced pressure. This resulted in Compound 95-5 (2 g, crude) as a white solid.

### Synthesis of Compound 95.

To a stirred solution of Compound 95 (1 g, 1.881 mmol, 1 equiv) and Intermediate G (0.86 g, 1.881 mmol, 1 equiv) in DCM (10 mL) were added Ti(Oi-Pr)₄ (2.14 g, 7.524 mmol) at room temperature under nitrogen atmosphere. The resulting mixture was stirred for overnight at room temperature under nitrogen atmosphere. To the above mixture was added NaBH(OAc)₃ (1.20 g, 5.643 mmol, 3 equiv) at room temperature. The resulting mixture was stirred for additional 2 h at room temperature. The reaction was quenched with 10% AcOH (aq.) at room temperature. The aqueous layer was extracted with EtOAc (1×100 mL). The resulting mixture was filtered, the filter cake was washed with water (20 mL). The filtrate was concentrated under reduced pressure. The residue was purified by reverse flash chromatography with the following conditions: column, C18 silica gel; mobile phase, MeCN in Water (0.1% FA), 10% to 50% gradient in 25 min; detector, UV 254 nm. The crude product (180 mg) was purified by Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 11% B to 22% B in 7 min, 22% B; Wave Length: 254; 220 nm; RT1 (min): 6.98) to afford Compound 95 (69.8 mg, 3.88%) as a yellow solid.

LCMS-Compound 95: (ES, m/z): [M+H] 971

NMR-Compound 95: (400 MHz, DMSO-d6, δ ppm): 1.21-1.25 (m, 2H), 1.67-1.69 (m, 1H), 1.71-1.85 (m, 6H), 1.94-2.08 (m, 5H), 2.34-2.50 (m, 2H), 2.54-2.60 (m, 3H), 2.87-2.90 (m, 2H), 3.23-3.33 (m, 7H), 3.36-3.49 (m, 7H), 4.17-4.30 (m, 3H), 4.34-4.49 (m, 2H), 5.02-5.07 (m, 1H), 7.01-7.05 (m, 3H), 7.18-7.20 (m, 1H), 7.27-7.33 (m, 5H), 7.42-7.44 (t, 1H), 7.46-7.50 (m, 1H), 7.67-7.75 (m, 3H), 8.32 (s, 1H), 10.95 (s, 1H).

### Example 94. Compound 96

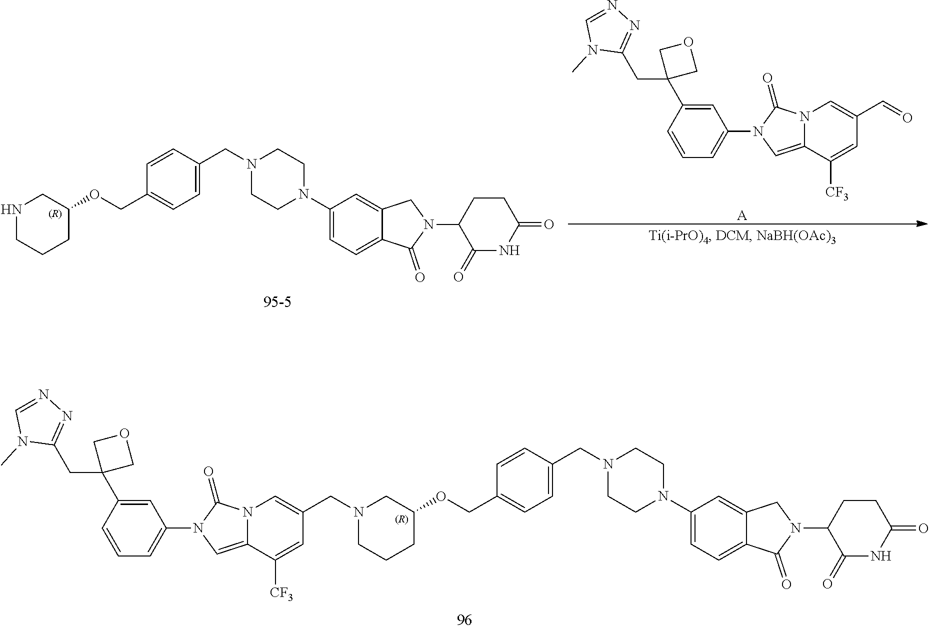

### Synthesis of Compound 96.

To a stirred solution of Compound 95-5 (1 g, 1.88 mmol, 1.0 equiv) and Intermediate A (1.3 g, 2.82 mmol, 1.5 equiv) in DCM (10 mL) was added Ti(Oi-Pr)₄ (2.1 g, 7.52 mmol, 4.0 equiv) at room temperature. The resulting mixture was stirred for overnight at room temperature. To the above mixture was added NaBH(OAc)₃ (1.2 g, 5.64 mmol, 3.0 equiv) at room temperature. The resulting mixture was stirred for additional 2 h at room temperature. The reaction was quenched by the addition of 10% AcOH (100 mL) at room temperature. The aqueous layer was extracted with EtOAc (2×50 mL). The aqueous phase was concentrated under reduced pressure. The residue was purified by reversed-phase flash chromatography with the following conditions: column, C18 silica gel; mobile phase, MeCN in water (0.1% FA), 10% to 50% gradient in 30 min. The crude product (400 mg) was purified by Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 8% B to 20% B in 8 min, 20% B; Wave Length: 254; 220 nm; RT1 (min): 7.5) to afford Compound 96 (100 mg, 5%) as a yellow solid.

LC-MS-Compound 96: (ES, m/z): [M+H]⁺ 973

H-NMR-Compound 96: (400 MHz, DMSO-d6, δ ppm): 1.23-1.71 (m, 4H), 1.94-2.03 (m, 4H), 2.33-2.37 (m, 3H), 2.51-2.60 (m, 4H), 2.87-2.90 (m, 2H), 2.96 (s, 3H), 3.27-3.31 (m, 3H), 3.42-3.44 (m, 2H), 3.49-3.53 (m, 5H), 4.17-4.21 (d, 1H), 4.30-4.34 (d, 1H), 4.48-4.52 (m, 2H), 4.90-4.95 (m, 4H), 5.02-5.07 (m, 1H), 6.87-6.89 (d, 1H), 7.01-7.05 (m, 3H), 7.27-7.33 (m, 4H), 7.37-7.39 (m, 1H), 7.41-7.42 (m, 2H), 7.50-7.52 (d, 1H), 7.67 (s, 1H), 7.75-7.78 (d, 1H), 8.20 (s, 1H), 10.95 (s, 1H).

### Example 95. Compound 97

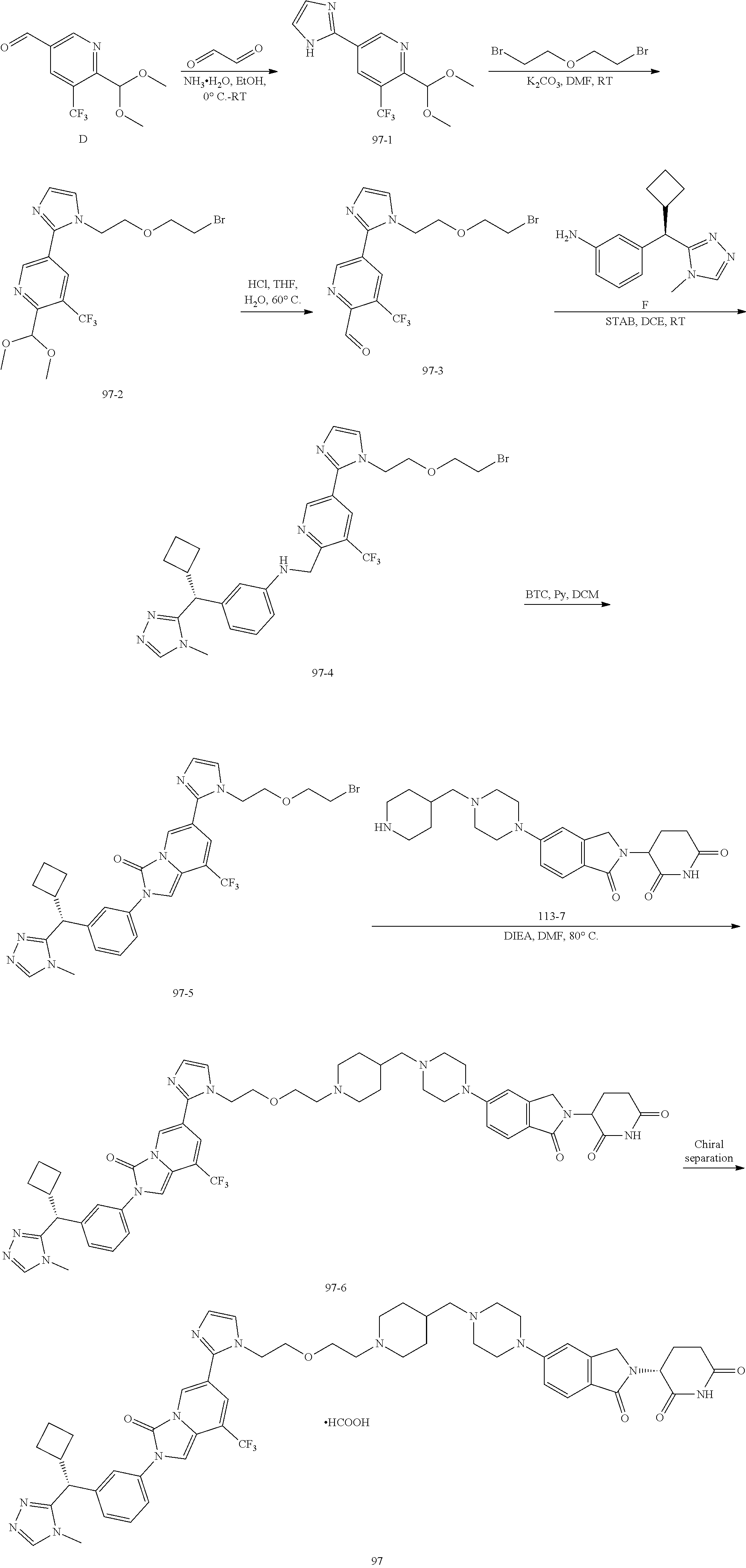

### Synthesis of Compound 97-1.

To a stirred solution of Intermediate D (20 g, 80.260 mmol, 1 equiv) and glyoxal (23.29 g, 401.300 mmol, 5 equiv) in EtOH (50 mL) was added NH₃·H₂O (74.02 g, 802.600 mmol, 10 equiv, 38%) at room temperature. The resulting mixture was stirred overnight at room temperature. The resulting mixture was concentrated under vacuum. The residue was purified by silica gel column chromatography, eluted with DCM/MeOH (30:1) to afford the crude product. The crude product was purified by reverse flash chromatography with the following conditions: column, C18 silica gel; mobile phase, MeCN in Water (10 mmol/L NH₄HCO₃), 0% to 50% gradient in 20 min; detector, UV 220 nm. This resulted in Compound 97-1 (3.8 g, 15.66%) as an off-white solid.

### Synthesis of Compound 97-2.

To a stirred solution of Compound 97-1 (3.8 g, 13.229 mmol, 1 equiv) and 1-bromo-2-(2-bromoethoxy)ethane (30.68 g, 132.290 mmol, 10.00 equiv) in DMF (100 mL) was added K₂CO₃ (14.63 g, 105.832 mmol, 8 equiv) at room temperature. The resulting mixture was stirred for two days at room temperature. The resulting mixture was diluted with water (300 mL). The aqueous layer was extracted with EtOAc (2×100 mL). The resulting mixture was concentrated under vacuum. The residue was purified by silica gel column chromatography, eluted with DCM/MeOH (100:1) to afford Compound 97-2 (5 g, 81.07%) as a light yellow oil.

### Synthesis of Compound 97-3.

To a stirred solution of Compound 97-2 (2.5 g, 5.705 mmol, 1 equiv) in THF (4 mL)/H₂O (10 mL) was added con. HCl (3 mL) at room temperature. The resulting mixture was stirred overnight at 60° C. The mixture was basified to pH 8 with saturated Na₂CO₃ (aq.). The aqueous layer was extracted with EtOAc (3×80 mL). The resulting mixture was concentrated under vacuum. The residue was purified by silica gel column chromatography, eluted with DCM/MeOH (50:1) to afford Compound 97-3 (1.52 g, 63.19%) as a light yellow oil.

### Synthesis of Compound 97-4.

A solution of Compound 97-3 (1.5 g, 3.825 mmol, 1 equiv) and Intermediate F (0.93 g, 3.825 mmol, 1 equiv) in DCE (20 mL) was stirred for 4 h at room temperature. To the above mixture was added STAB (1.62 g, 7.650 mmol, 2 equiv) at room temperature. The resulting mixture was stirred for additional 4 h at room temperature. The reaction was quenched with saturated NaHCO₃ (aq.) at room temperature. The aqueous layer was extracted with DCM (3×80 mL). The residue was purified by silica gel column chromatography, eluted with DCM/MeOH (30:1) to afford Compound 97-4 (1.3 g, 51.66%) as a light yellow solid.

### Synthesis of Compound 97-5.

To a stirred solution of Compound 97-4 (1.3 g, 2.102 mmol, 1 equiv) and pyridine (1.66 g, 21.020 mmol, 10 equiv) in DCM (30 mL) was added triphosgene (0.25 g, 0.841 mmol, 0.4 equiv) at room temperature. The resulting mixture was stirred for 10 min at room temperature. The reaction was quenched with saturated NaHCO₃ (aq.) (200 mL) at room temperature. The aqueous layer was extracted with DCM (3×50 mL). The resulting mixture was concentrated under vacuum. The residue was purified by Prep-TLC (DCM/MeOH 15:1) to afford Compound 97-5 (800 mg, 55.51%) as a yellow solid.

### Synthesis of Compound 97-6.

To a stirred solution of Compound 97-5 (400 mg, 0.621 mmol, 1 equiv) and Compound 113-7 (396.15 mg, 0.931 mmol, 1.5 equiv) in DMF (10 mL) were added DIEA (240.65 mg, 1.863 mmol, 3 equiv) room temperature. The resulting mixture was stirred overnight at 80° C. The residue was purified by reverse flash chromatography with the following conditions (column, C18 silica gel; mobile phase, MeCN in Water (0.1% FA), 10% to 50% gradient in 30 min; detector, UV 254 nm) to afford the crude product. The crude product was purified by Prep-HPLC with the following conditions (Column: Xselect CSH C18 OBD Column 30*150 mm 5 μm, n; Mobile Phase A: Water (0.1% FA) Water (0.1% FA), Mobile Phase B: ACN; Flow rate: 60 mL/min; Gradient: 5% B to 18% B in 9 min, Wave Length: 254; 220 nm; RT1 (min): 8.78) to afford Compound 97-6 (60 mg, 9.48%) as a yellow solid.

### Synthesis of Compound 97.

The Compound 97-6 (60 mg) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 60% B to 60% B in 25 min; Wave Length: 220/254 nm; RT1 (min): 8.51; RT2 (min): 16.99; the first peak is product) to afford the crude product. The crude product was purified by reverse flash chromatography with the following conditions: column, C18 silica gel; mobile phase, MeCN in water (0.1% FA), 10% to 50% gradient in 10 min; detector, UV 254 nm. This resulted in Compound 97 (22.8 mg, 34.49%) as a yellow solid.

LC-MS-Compound 97: (ES, m/z): [M−HCOOH+H]⁺ 989

H-NMR-Compound 97: (400 MHz, CD₃OD, δ ppm): 1.23-1.41 (m, 2H), 1.61-1.92 (m, 8H), 1.98-2.19 (m, 4H), 2.31-2.45 (m, 1H), 2.45-2.61 (m, 4H), 2.65-2.78 (m, 1H), 2.78-2.88 (m, 3H), 3.31-3.32 (m, 3H), 3.36-3.38 (m, 2H), 3.38-3.42 (m, 2H), 3.44 (s, 3H), 3.75-3.78 (m, 2H), 3.88-3.93 (m, 2H), 4.18-4.31 (m, 5H), 4.97-5.02 (m, 1H), 6.97-7.05 (m, 3H), 7.15-7.28 (m, 4H), 7.39-7.43 (m, 1H), 7.52-7.59 (m, 3H), 8.26 (s, 1H), 8.31 (s, 1H), 8.36 (s, 1H).

### Example 96. Compound 98

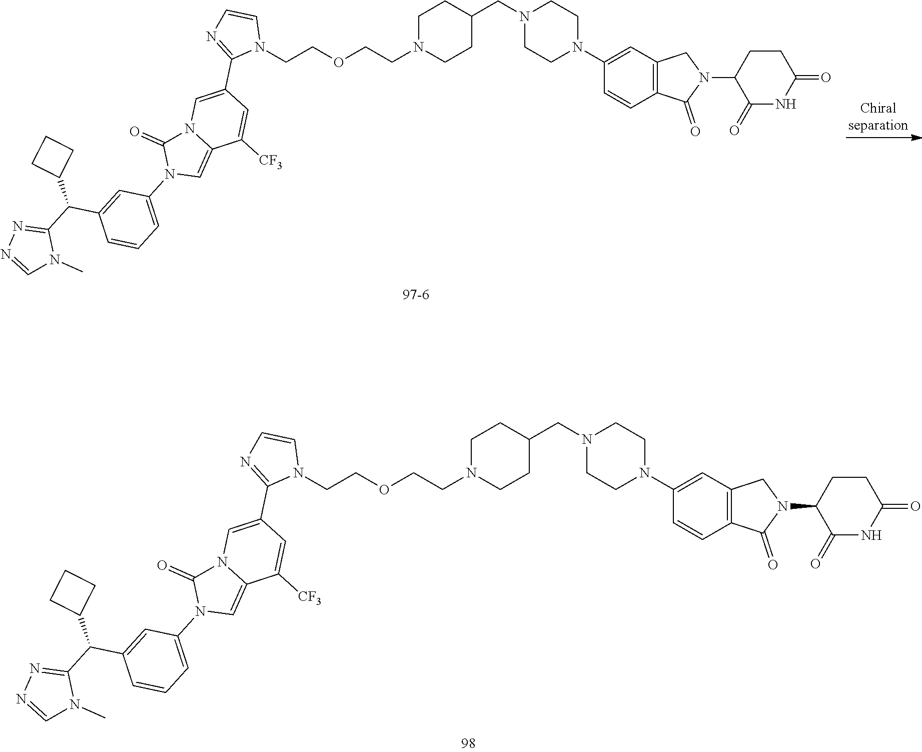 [SMILES: Cn1cnnc1[C@@H](c1cccc(-n2cc3c(C(F)(F)F)cc(-c4nccn4CCOCCN4CCC(CN5CCN(c6ccc7c(c6)CN(C6CCC(=O)NC6=O)C7=O)CC5)CC4)cn3c2=O)c1)C1CCC1 ; Cn1cnnc1[C@@H](c1cccc(-n2cc3c(C(F)(F)F)cc(-c4nccn4CCOCCN4CCC(CN5CCN(c6ccc7c(c6)CN([C@H]6CCC(=O)NC6=O)C7=O)CC5)CC4)cn3c2=O)c1)C1CCC1]

### Synthesis of Compound 98.

The Compound 97-6 (60 mg) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 60% B to 60% B in 25 min; Wave Length: 220/254 nm; RT1 (min): 8.51; RT2 (min): 16.99; the second peak is product) to afford the crude product. The crude product was purified by reverse flash chromatography with the following conditions: column, C18 silica gel; mobile phase, MeCN in water (0.1% FA), 10% to 50% gradient in 10 min; detector, UV 254 nm. This resulted in Compound 98 (16.1 mg, 26.03%) as a yellow solid.

LC-MS-Compound 98: (ES, m/z): [M+H]⁺ 989

H-NMR-Compound 98: (400 MHz, CD₃OD, δ ppm): 1.15-1.31 (m, 2H), 1.51-1.61 (m, 1H), 1.61-1.87 (m, 7H), 1.98-2.11 (m, 4H), 2.24-2.43 (m, 7H), 2.65-2.75 (m, 1H), 2.78-2.92 (m, 3H), 3.02-3.11 (m, 2H), 3.31-3.32 (m, 5H), 3.44 (s, 3H), 3.62-3.65 (m, 2H), 3.80-3.87 (m, 2H), 4.17-4.30 (m, 5H), 4.97-5.02 (m, 1H), 6.94-7.03 (m, 3H), 7.14-7.27 (m, 4H), 7.38-7.42 (m, 1H), 7.51-7.58 (m, 3H), 8.26 (s, 1H), 8.30 (s, 1H).

### Example 97. Compound 99

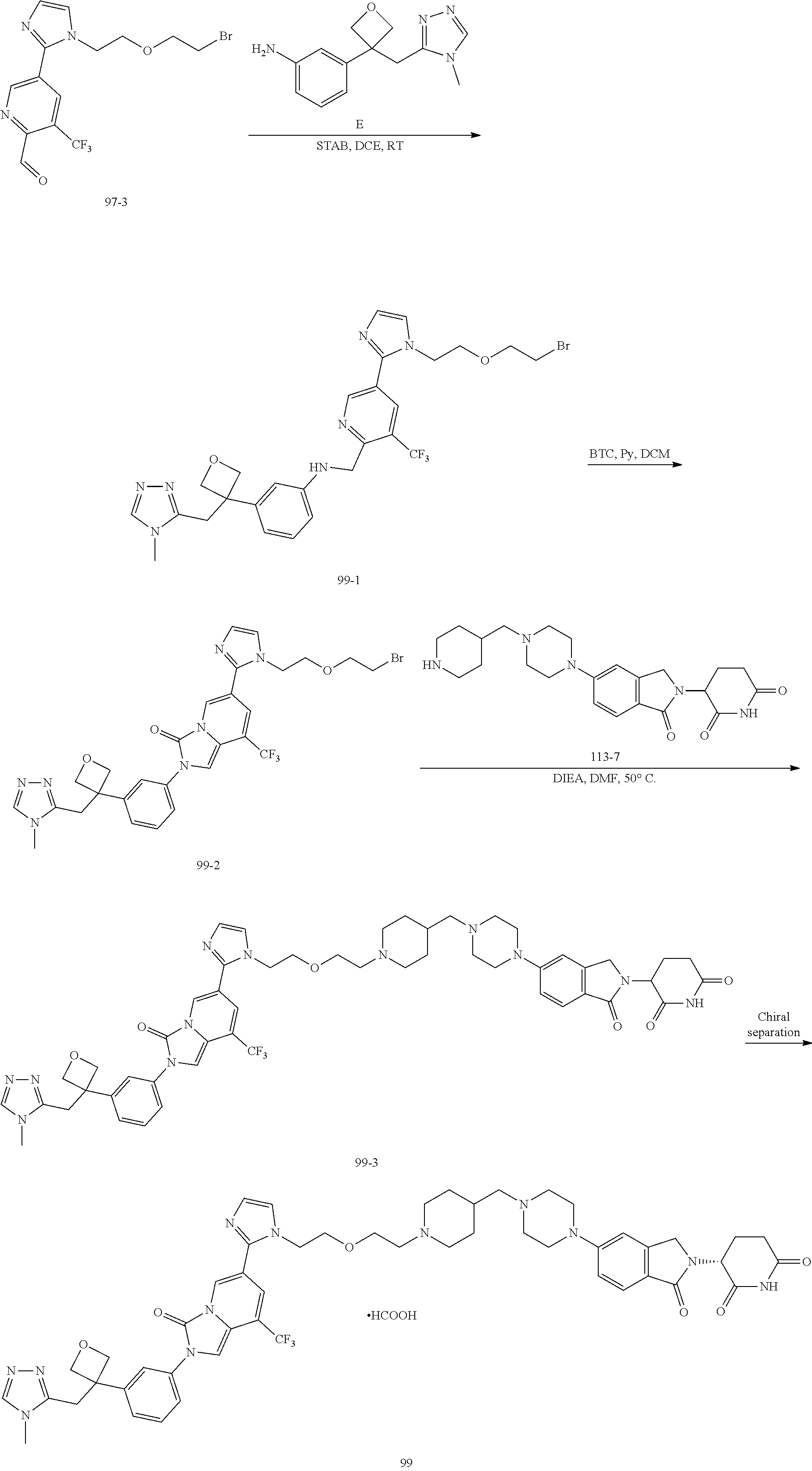

### Synthesis of Compound 99-1.

A solution of Compound 97-3 (1.5 g, 3.825 mmol, 1 equiv) and Intermediate E (0.93 g, 3.825 mmol, 1 equiv) in DCE (20 mL) was stirred for 4 h at room temperature. To the above mixture was added STAB (1.62 g, 7.650 mmol, 2 equiv) at room temperature. The resulting mixture was stirred for additional 4 h at room temperature. The reaction was quenched with saturated NaHCO₃ (aq.) (0.150 mL) at room temperature. The aqueous layer was extracted with DCM (3×80 mL). The residue was purified by silica gel column chromatography, eluted with DCM/MeOH (30:1) to afford Compound 99-1 (1.27 g, 50.30%) as a light yellow solid.

### Synthesis of Compound 99-2.

To a stirred solution of Compound 99-1 (1.25 g, 2.015 mmol, 1 equiv.) and pyridine (1.59 g, 20.150 mmol, 10 equiv) in DCM (25 mL) was added triphosgene (0.24 g, 0.806 mmol, 0.4 equiv) at room temperature. The resulting mixture was stirred for 10 min at room temperature. The reaction was quenched with saturated NaHCO₃ (aq.) (150 mL) at room temperature. The aqueous layer was extracted with DCM (3×50 mL). The resulting mixture was concentrated under vacuum. The residue was purified by Prep-TLC (DCM/MeOH 15:1) to afford Compound 99-2 (0.8 g, 57.74%) as a yellow solid.

### Synthesis of Compound 99-3.

To a stirred solution of Compound 99-2 (400 mg, 0.619 mmol, 1 equiv) and Compound 113-7 (394.95 mg, 0.928 mmol, 1.5 equiv) in DMF (10 mL) were added DIEA (239.91 mg, 1.857 mmol, 3 equiv) room temperature. The resulting mixture was stirred overnight at 60° C. The residue was purified by reverse flash chromatography with the following conditions (column, C18 silica gel; mobile phase, MeCN in Water (0.1% FA), 10% to 50% gradient in 30 min; detector, UV 254 nm) to afford the crude product. The crude product was purified by Prep-HPLC with the following conditions (Column: Xselect CSH C18 OBD Column 30*150 mm 5 μm, n; Mobile Phase A: Water (0.1% FA) Water (0.1% FA), Mobile Phase B: ACN; Flow rate: 60 mL/min; Gradient: 3% B to 13% B in 10 min, Wave Length: 254; 220 nm; RT1 (min): 9.98) to afford Compound 99-3 (60 mg, 9.39%) as a yellow solid.

### Synthesis of Compound 99.

The Compound 99-3 (60 mg) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 80% B to 80% B in 25 min; Wave Length: 220/254 nm; RT1 (min): 2.13; RT2 (min): 19.65; the first peak is product) to afford the crude product. The crude product was purified by reverse flash chromatography with the following conditions: column, C18 silica gel; mobile phase, MeCN in water (0.1% FA), 10% to 40% gradient in 10 min; detector, UV 254 nm. This resulted in Compound 99 (17.1 mg, 26.96%) as a yellow solid.

LC-MS-Compound 99: (ES, m/z): [M−HCOOH+H]⁺ 991

H-NMR-Compound 99: (400 MHz, CD₃OD, δ ppm): 1.31-1.41 (m, 2H), 1.69-1.92 (m, 3H), 1.98-2.10 (m, 1H), 2.13-2.15 (m, 2H), 2.31-2.42 (m, 1H), 2.45-2.52 (m, 4H), 2.65-2.73 (m, 1H), 2.75-2.85 (m, 3H), 2.87 (s, 3H), 3.21-3.32 (m, 6H), 3.31-3.42 (m, 2H), 3.55 (s, 2H), 3.75-3.95 (m, 4H), 4.24-4.30 (m, 4H), 4.94-5.02 (m, 5H), 6.91-6.97 (m, 3H), 7.05 (s, 1H), 7.14 (s, 1H), 7.20-7.22 (m, 2H), 7.28 (s, 1H), 7.38-7.42 (m, 1H), 7.51-7.56 (m, 2H), 8.11 (s, 1H), 8.33 (s, 1H), 8.40 (s, 1H).

### Example 98. Compound 100

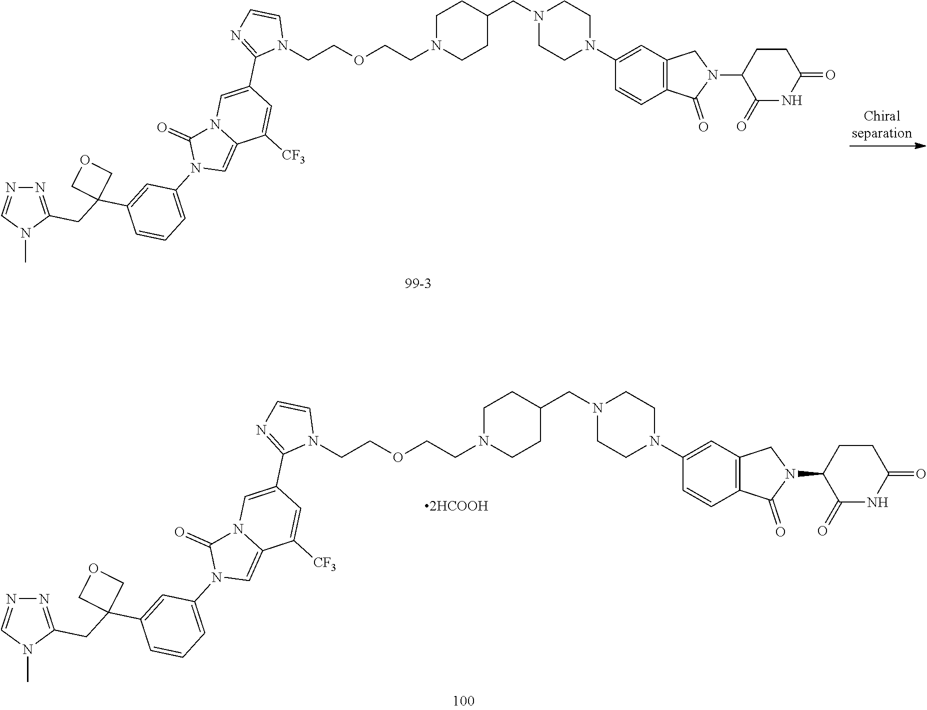 [SMILES: Cn1cnnc1CC1(c2cccc(-n3cc4c(C(F)(F)F)cc(-c5nccn5CCOCCN5CCC(CN6CCN(c7ccc8c(c7)CN(C7CCC(=O)NC7=O)C8=O)CC6)CC5)cn4c3=O)c2)COC1 ; Cn1cnnc1CC1(c2cccc(-n3cc4c(C(F)(F)F)cc(-c5nccn5CCOCCN5CCC(CN6CCN(c7ccc8c(c7)CN([C@H]7CCC(=O)NC7=O)C8=O)CC6)CC5)cn4c3=O)c2)COC1]

### Synthesis of Compound 100.

The Compound 99-3 (60 mg) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 80% B to 80% B in 25 min; Wave Length: 220/254 nm; RT1 (min): 2.13; RT2 (min): 19.65; the second peak is product) to afford the crude product. The crude product was purified by reverse flash chromatography with the following conditions: column, C18 silica gel; mobile phase, MeCN in water (0.1% FA), 10% to 40% gradient in 10 min; detector, UV 254 nm. This resulted in Compound 100 (20.1 mg, 30.35%) as a yellow solid.

LC-MS-Compound 100: (ES, m/z): [M−2HCOOH+H]⁺ 991

H-NMR-Compound 100: (400 MHz, CD₃OD, δ ppm): 1.31-1.45 (m, 2H), 1.73-1.93 (m, 3H), 1.98-2.10 (m, 1H), 2.15-2.23 (m, 2H), 2.29-2.42 (m, 1H), 2.45-2.54 (m, 4H), 2.66-2.75 (m, 1H), 2.75-2.95 (m, 6H), 3.21-3.45 (m, 8H), 3.55 (s, 2H), 3.77-3.95 (m, 4H), 4.24-4.30 (m, 4H), 4.94-5.01 (m, 5H), 6.91-6.97 (m, 3H), 7.05 (s, 1H), 7.14 (s, 1H), 7.19-7.22 (m, 2H), 7.29 (s, 1H), 7.38-7.42 (m, 1H), 7.51-7.56 (m, 2H), 8.11 (s, 1H), 8.26 (s, 2H), 8.33 (s, 1H).

### Example 99. Compound 101

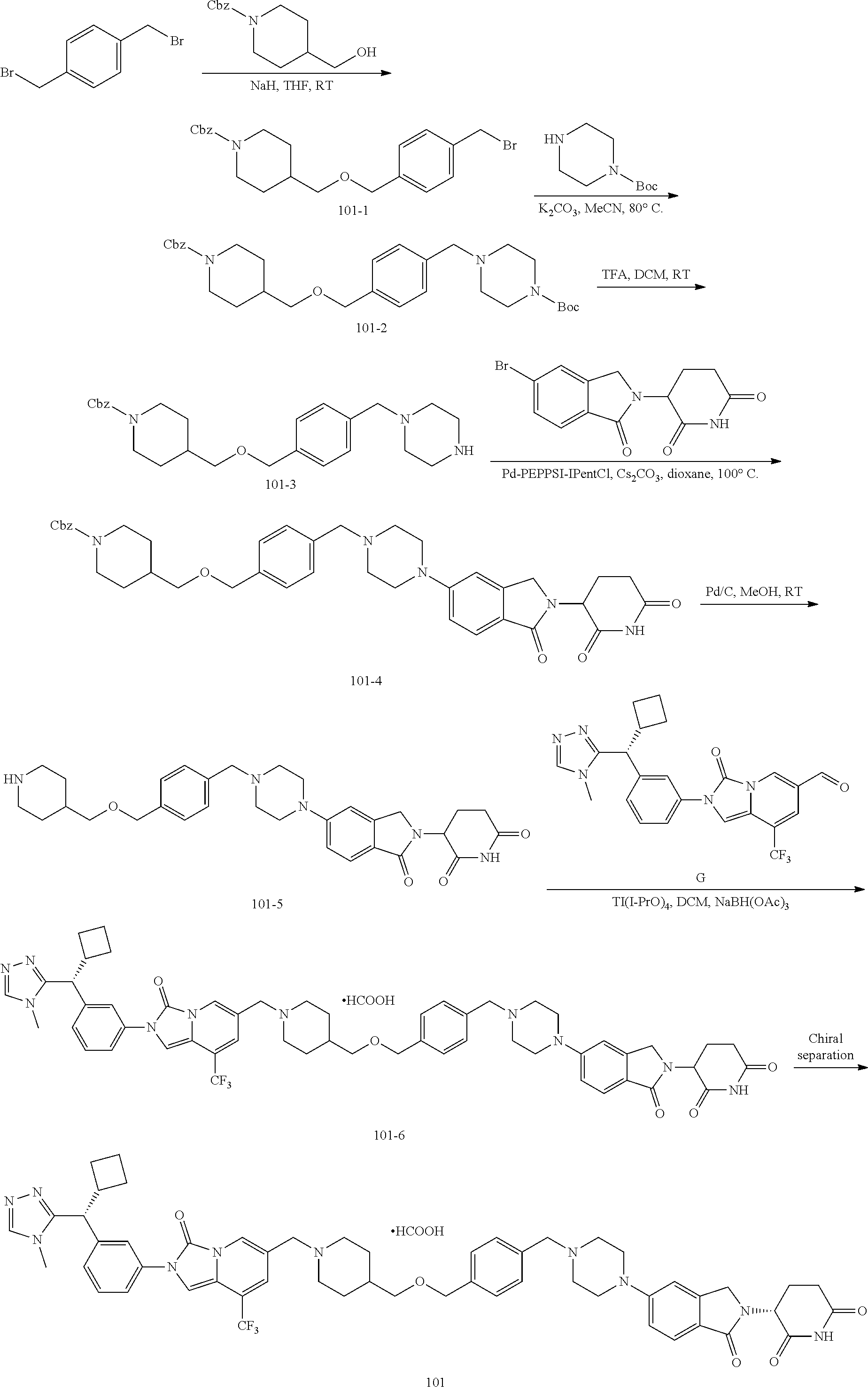

### Synthesis of Compound 101-1.

To a solution of benzyl 4-(hydroxymethyl)piperidine-1-carboxylate (20 g, 80.221 mmol, 1 equiv) in THF (250 mL) was added NaH (4.81 g, 120.332 mmol, 1.5 equiv, 60%) at 0 degrees C. The resulting mixture was stirred for 1 h at 0° C. under nitrogen atmosphere. To the above mixture was added benzene, 1,4-bis(bromomethyl)benzene (25.41 g, 96.265 mmol, 1.2 equiv) at 0° C. The resulting mixture was stirred overnight at room temperature. The reaction mixture was quenched with NH₄Cl (aq.) (300 mL) and extracted with DCM (3×250 mL). The resulting mixture was concentrated under reduced pressure. The residue was purified by silica gel column chromatography, eluted with PE/EA (5:1) to afford Compound 101-1 (17 g, 44.60%) as a light yellow oil.

### Synthesis of Compound 101-2.

To a stirred solution of Compound 101-1 (17 g, 39.319 mmol, 1 equiv) and tert-butyl piperazine-1-carboxylate (10.99 g, 59.005 mmol, 1.50 equiv) in MeCN (200 mL) was added K₂CO₃ (10.87 g, 78.638 mmol, 2 equiv) at room temperature. The resulting mixture was stirred for 6 h at 80° C. The resulting mixture was diluted with water (800 mL). The aqueous layer was extracted with EtOAc (3×300 mL). The resulting mixture was concentrated under vacuum. The residue was purified by silica gel column chromatography, eluted with PE/EA (2:1) to afford Compound 101-2 (19 g, 79.08%) as a light yellow oil.

### Synthesis of Compound 101-3.

A solution of Compound 101-2 (17 g, 31.616 mmol, 1 equiv) and TFA (40 mL) in DCM (200 mL) was stirred for 2 h at room temperature. The resulting mixture was concentrated under reduced pressure. The mixture was neutralized to pH 8 with saturated NaHCO₃ (aq.). The aqueous layer was extracted with DCM (5×300 mL). The resulting mixture was concentrated under vacuum. The residue was purified by reverse flash chromatography with the following conditions: column, C18 silica gel; mobile phase, MeCN in water (10 mmol/L NH₄HCO₃), 0% to 50% gradient in 20 min; detector, UV 220 nim. This resulted in Compound 101-3 (8 g, 54.93%) as a light yellow oil.

### Synthesis of Compound 101-4.

To a stirred solution of Compound 101-3 (5 g, 11.426 mmol, 1 equiv) and 3-(5-bromo-1-oxo-3H-isoindol-2-yl)piperidine-2,6-dione (7.38 g, 22.852 mmol, 2 equiv) in dioxane (150 mL) was added Cs₂CO₃ (11.17 g, 34.278 mmol, 3 equiv) and Pd-PEPPSI-IPentCl 2-methylpyridine (o-picoline) (0.96 g, 1.143 mmol, 0.1 equiv) at room temperature under nitrogen atmosphere. The resulting mixture was stirred overnight at 100° C. under nitrogen atmosphere. The mixture was allowed to cool down to room temperature. The mixture/residue was acidified to pH 6 with HOAc. The resulting mixture was concentrated under vacuum. The residue was purified by silica gel column chromatography, eluted with DCM (0.1% HOAc)/MeOH (20:1) to afford Compound 101-3 (3.5 g, 39.65%) as a light brown solid.

### Synthesis of Compound 101-5.

To a solution of Compound 101-4 (1.5 g, 2.206 mmol, 1 equiv) in MeOH (80 mL) was added Pd/C (10%, 0.15 g) under nitrogen atmosphere in a 250 mL round-bottom flask. The mixture was hydrogenated at room temperature for 3 h under hydrogen atmosphere using a hydrogen balloon, filtered through a Celite pad and concentrated under reduced pressure. This resulted in Compound 101-5 (1.2 g, 84.72%) as a light brown solid.

### Synthesis of Compound 101-6.

To a stirred solution of Compound 101-5 (1.2 g, 2.199 mmol, 1 equiv) and Intermediate G (1.00 g, 2.199 mmol, 1 equiv) in DCM (20 mL) was added Ti(Oi-Pr)₄ (1.88 g, 6.597 mmol, 3 equiv) at room temperature. The resulting mixture was stirred overnight at room temperature. To the above mixture was added NaBH(OAc)₃ (1.86 g, 8.796 mmol, 4 equiv) at room temperature. The resulting mixture was stirred for additional 6 h at room temperature. The reaction was quenched with 10% HOAc (aq.) (150 mL) at room temperature. The aqueous layer was extracted with EtOAc (50 mL). The aqueous layer was filtered, the filter cake was washed with water (10 mL). The filtrate was concentrated under reduced pressure. The residue was purified by reverse flash chromatography with the following conditions: column, C18 silica gel; mobile phase. MeCN in water (0.1% FA), 10% to 30% gradient in 20 min; detector, UV 254 nm. This resulted in Compound 101-6 (700 mg, 31.34%) as a yellow solid.

### Synthesis of Compound 101.

The Compound 101-6 (450 mg) was purified by Prep-Chiral-HPLC with the following conditions (Column: CHIRALPAK IH, 2*25 cm, 5 μm; Mobile Phase A: MTBE (0.1% TEA), Mobile Phase B: IPA:DCM=1:1; Flow rate: 20 mL/min; Gradient: 60% B to 60% B in 20 min; Wave Length: 220/254 nm; RT1 (min): 5.41; RT2 (min): 13.88; the first peak is product) to afford the crude product. The crude product was purified by reverse flash chromatography with the following conditions: column, C18 silica gel; mobile phase, MeCN in water (0.1% FA), 10% to 50% gradient in 20 min; detector, UV 254 nm. This resulted in Compound 101 (101.6 ng, 24.00%) as a yellow solid.

LC-MS-Compound 101: (ES, m/z): [M−HCOOH+H]⁺ 985

H-NMR-Compound 101: (400 MHz, DMSO-d6, δ ppm): 1.15-1.25 (m, 2H), 1.54-1.65 (m, 1H), 1.65-1.75 (m, 3H), 1.75-1.88 (m, 4H), 1.94-2.03 (m, 3H), 2.03-2.15 (m, 1H), 2.33-2.41 (m, 1H), 2.51-2.60 (m, 1H), 2.84-2.94 (m, 3H), 3.15-3.35 (m, 13H), 3.43 (s, 3H), 3.52 (s, 2H), 4.17-4.34 (m, 3H), 4.44 (s, 2H), 5.03-5.07 (m, 1H), 7.00-7.05 (m, 3H), 7.18-7.20 (d, 1H), 7.27-7.31 (m, 5H), 7.42-7.46 (m, 1H), 7.50-7.52 (d, 1H), 7.68-7.73 (m, 3H), 8.15 (s, 1H), 8.33 (s, 1H), 10.95 (s, 1H).

### Example 100. Compound 102

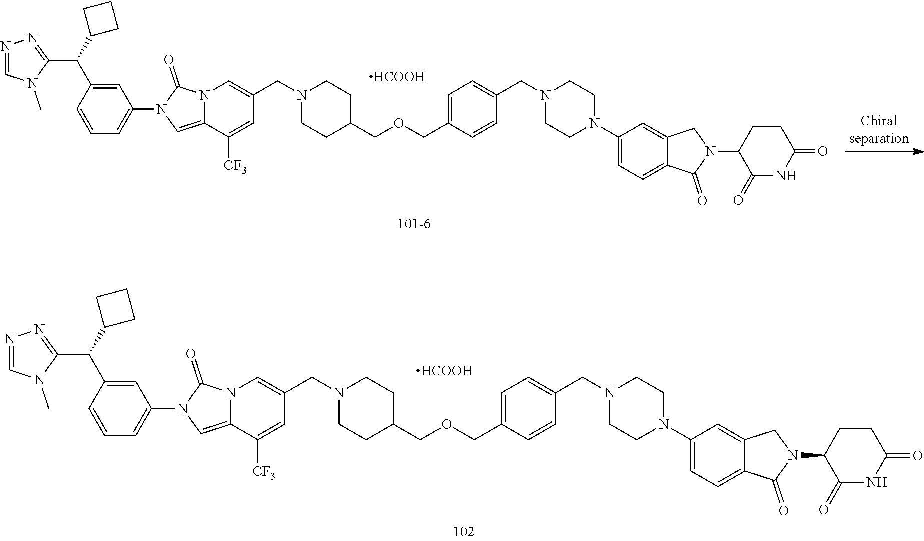 [SMILES: Cn1cnnc1[C@@H](c1cccc(-n2cc3c(C(F)(F)F)cc(CN4CCC(COCc5ccc(CN6CCN(c7ccc8c(c7)CN(C7CCC(=O)NC7=O)C8=O)CC6)cc5)CC4)cn3c2=O)c1)C1CCC1 ; "·HCOOH" ; Cn1cnnc1[C@@H](c1cccc(-n2cc3c(C(F)(F)F)cc(CN4CCC(COCc5ccc(CN6CCN(c7ccc8c(c7)CN([C@H]7CCC(=O)NC7=O)C8=O)CC6)cc5)CC4)cn3c2=O)c1)C1CCC1 ; "·HCOOH"]

### Synthesis of Compound 102.

The Compound 101-6 (450 mg) was purified by Prep-Chiral-HPLC with the following conditions (Column: CHIRALPAK IH, 2*25 cm, 5 μm; Mobile Phase A: MTBE (0.1% TEA), Mobile Phase B: IPA:DCM=1:1; Flow rate: 20 mL/min; Gradient: 60% B to 60% B in 20 min; Wave Length: 220/254 nm; RT1 (min): 5.41; RT2 (min): 13.88; the second peak is product) to afford the crude product. The crude product was purified by reverse flash chromatography with the following conditions: column, C18 silica gel; mobile phase, MeCN in water (0.1% FA), 10% to 50% gradient in 20 min; detector, UV 254 nm. This resulted in Compound 102 (77.1 mg, 18.27%) as a yellow solid.

LC-MS-Compound 102: (ES, m/z): [M−HCOOH+H]⁺ 985

H-NMR-Compound 102: (400 MHz, DMSO-d6, δ ppm): 1.15-1.25 (m, 2H), 1.54-1.65 (m, 1H), 1.65-1.75 (m, 3H), 1.75-1.88 (m, 4H), 1.94-2.03 (m, 3H), 2.03-2.15 (m, 1H), 2.33-2.41 (m, 1H), 2.51-2.60 (m, 1H), 2.84-2.94 (m, 3H), 3.15-3.35 (m, 13H), 3.43 (s, 3H), 3.52 (s, 2H), 4.17-4.34 (m, 3H), 4.44 (s, 2H), 5.03-5.07 (m, 1H), 7.00-7.05 (m, 3H), 7.18-7.20 (d, 1H), 7.27-7.31 (m, 5H), 7.42-7.46 (m, 1H), 7.50-7.52 (d, 1H), 7.68-7.73 (m, 3H), 8.15 (s, 1H), 8.33 (s, 1H), 10.95 (s, 1H).

### Example 101. Compound 103

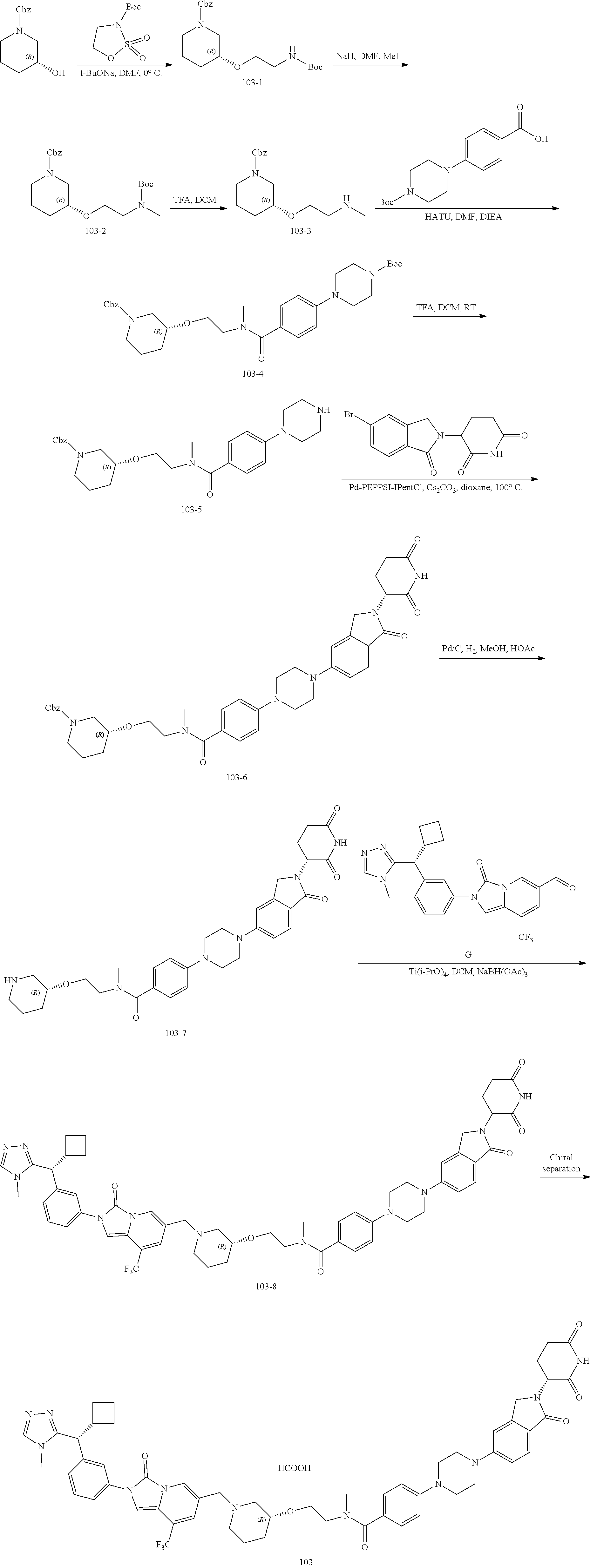

### Synthesis of Compound 103-1.

To a stirred mixture of benzyl (3R)-3-hydroxypiperidine-1-carboxylate (10 g, 42.502 mmol, 1 equiv) and tert-butyl 2,2-dioxo-1,2lambda6,3-oxathiazolidine-3-carboxylate (11.39 g, 51.002 mmol, 1.2 equiv) in DMF (100 mL) was added t-BuONa (8.17 g, 85.004 mmol, 2 equiv) in portions at 0° C. The resulting mixture was stirred overnight at room temperature. The mixture was neutralized to pH 7 with 4M HCl (aq). The aqueous layer was extracted with EtOAc (3×100 mL). The resulting mixture was concentrated under vacuum. The residue was purified by silica gel column chromatography, eluted with CH₂Cl₂/MeOH (100:1) to afford Compound 103-1 (16 g, 99.47%) as a light yellow oil.

### Synthesis of Compound 103-2.

To a stirred mixture of Compound 103-1 (16 g, 42.276 mmol, 1 equiv) in DMF (200 mL) was added NaH (3.38 g, 84.552 mmol, 2 equiv, 60%) at 0° C. under nitrogen atmosphere. The resulting mixture was stirred for 1 h at room temperature under nitrogen atmosphere. To the above mixture was added MeI (7.20 g, 50.731 mmol, 1.2 equiv) at room temperature. The resulting mixture was stirred overnight at room temperature. The reaction was quenched with NH₄Cl (aq.) (600 mL) at room temperature. The aqueous layer was extracted with EtOAc (3×200 mL). The resulting mixture was concentrated under vacuum. The residue was purified by silica gel column chromatography, eluted with PE/EA (5:1) to afford Compound 103-2 (13 g, 78.35%) as a brown oil.

### Synthesis of Compound 103-3.

To a stirred solution of Compound 103-2 (12.9 g, 32.867 mmol, 1 equiv) in DCM (100 mL) was added TFA (33 mL) at room temperature. The resulting mixture was stirred for 1 h at room temperature. The resulting mixture was concentrated under vacuum. This resulted in Compound 103-3 (13 g, 97.40%) as a light yellow oil.

### Synthesis of Compound 103-4.

To a stirred mixture of 4-[4-(tert-butoxycarbonyl)piperazin-1-yl]benzoic acid (9.81 g, 32.013 mmol, 1 equiv), DIEA (16.55 g, 128.052 mmol, 4 equiv) and HATU (21.91 g, 57.623 mmol, 1.8 equiv) in DMF (130 mg) at room temperature. The resulting mixture was stirred for 1 h at room temperature. To the above mixture was added Compound 103-3 (13 g, 32.013 mmol, 1 equiv) at room temperature. The resulting mixture was stirred for additional 5 h at room temperature. The reaction was quenched with NH₄Cl (aq.) (400 mL) at room temperature. The aqueous layer was extracted with EtOAc (5×100 mL). The resulting mixture was concentrated under vacuum. The residue was purified by silica gel column chromatography, eluted with CH₂Cl₂/MeOH (100:1) to afford Compound 103-4 (7 g, 37.65%) as a brown oil.

### Synthesis of Compound 103-5.

To a stirred mixture of Compound 103-4 (7 g, 12.054 mmol, 1 equiv) in DCM (52 mL) was added TFA (18 mL) at room temperature. The resulting mixture was stirred for 1 h at room temperature. The resulting mixture was concentrated under vacuum. The residue was purified by reversed-phase flash chromatography with the following conditions: column, C18 silica gel; mobile phase, MeCN in water (10 mmol/L NH₄HCO₃), 10% to 100% gradient in 35 min; detector, UV 220 nm. This resulted in Compound 103-5 (3.4 g, 58.69%) as a light yellow oil.

### Synthesis of Compound 103-6.

To a stirred solution of Compound 103-5 (3.35 g, 6.970 mmol, 1 equiv) and 3-(5-bromo-1-oxo-3H-isoindol-2-yl)piperidine-2,6-dione (4.50 g, 13.940 mmol, 2 equiv) in dioxane (50 mL) was added Cs₂CO₃ (6.81 g, 20.910 mmol, 3 equiv) and Pd-PEPPSI-IPentCl 2-methylpyridine (o-picoline (0.59 g, 0.697 mmol, 0.1 equiv) at room temperature under nitrogen atmosphere. The resulting mixture was stirred overnight at 100° C. under nitrogen atmosphere. The mixture was allowed to cool down to room temperature. The mixture/residue was acidified to pH 6 with HOAc. The resulting mixture was concentrated under vacuum. The residue was purified by silica gel column chromatography, eluted with DCM (0.5% HOAc)/MeOH (20:1) to afford Compound 103-6 (1.3 g, 23.22%) as a light brown solid.

### Synthesis of Compound 103-7.

To a solution of Compound 103-6 (1.28 g, 1.771 mmol, 1 equiv) and HOAc (2 mL) in MeOH (100 mL) was added Pd/C (10%, 0.12 g) under nitrogen atmosphere in a 250 mL round-bottom flask. The mixture was hydrogenated at room temperature for 2 h under hydrogen atmosphere using a hydrogen balloon, filtered through a Celite pad and concentrated under reduced pressure. This resulted in Compound 103-7 (1 g, 95.93%) as a brown solid.

### Synthesis of Compound 103-8.

To a stirred solution of Compound 103-7 (980 mg, 1.665 mmol, 1 equiv) and Intermediate G (1061.42 mg, 2.331 mmol, 1.4 equiv) in DCM (20 mL) was added Ti(Oi-Pr)₄ (946.24 mg, 3.330 mmol, 2 equiv) at room temperature. The resulting mixture was stirred overnight at room temperature. To the above mixture was added NaBH(OAc)₃ (705.62 mg, 3.330 mmol, 2 equiv) at room temperature. The resulting mixture was stirred for additional 4 h at room temperature. The reaction was quenched with 10% HOAc (aq.) (200 mL) at room temperature. The resulting mixture was filtered, the filter cake was washed with 10% HOAc (aq.) (20 mL). The aqueous layer was extracted with EtOAc (2×100 mL). The aqueous layer was concentrated under vacuum. The residue was purified by reversed-phase flash chromatography with the following conditions: column, C18 silica gel; mobile phase, MeCN in water (0.1% FA), 0% to 30% gradient in 30 min; detector, UV 254 nm. This resulted in Compound 103-8 (600 mg, 35.06%) as a yellow solid.

### Synthesis of Compound 103.

The Compound 103 (400 mg) was purified by Prep-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 90% B to 90% B in 25.5 min; Wave Length: 220/254 nm; RT1 (min): 13.02; RT2 (min): 17.49, the first peak is product) to afford the crude product. The crude product was purified by reversed-phase flash chromatography with the following conditions: column, C18 silica gel; mobile phase, MeCN in water (0.1% FA), 0% to 30% gradient in 20 min; detector, UV 254 nm. This resulted in Compound 103 (79.2 mg, 19.8%) as a yellow solid.

LC-MS-Compound 103: (ES, m/z): [M−HCOOH+H]⁺ 1028

H-NMR-Compound 103: (400 MHz, DMSO-d4, δ ppm): 1.33-1.36 (m, 1H), 1.43-1.46 (m, 1H), 1.66-1.98 (m, 7H), 2.03-2.07 (m, 4H), 2.30-2.42 (m, 1H), 2.50-2.69 (m, 2H), 2.80-2.97 (m, 5H), 3.20-3.32 (m, 3H), 3.41-3.55 (m, 14H), 3.56-3.59 (m, 2H), 4.21-4.24 (m, 2H), 4.32-4.36 (m, 1H), 5.04-5.08 (d, 1H), 6.95-7.01 (m, 2H), 7.06 (s, 1H), 7.08-7.10 (m, 2H), 7.18-7.20 (d, 1H), 7.28-7.30 (m, 3H), 7.41-7.45 (m, 1H) 7.54-7.56 (m, 1H), 7.67-7.70 (m, 2H), 7.70-7.74 (m, 1H), 8.14 (s, 1H), 8.32 (s, 1H), 10.95 (s, 1H).

### Example 102. Compound 104

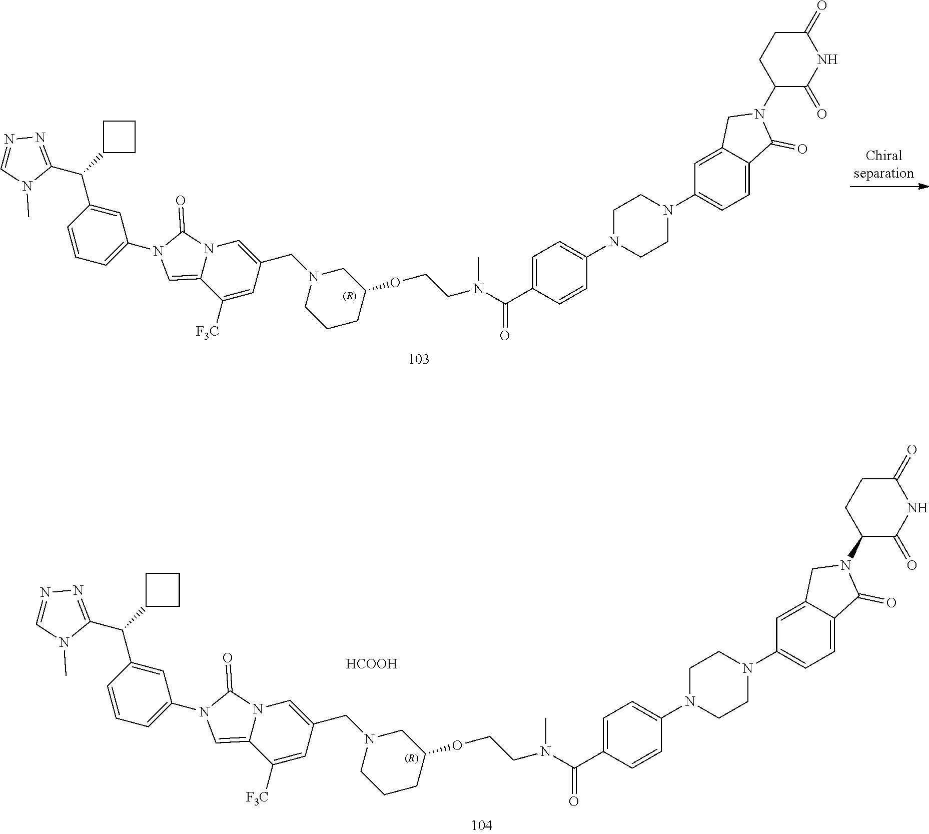

### Synthesis of Compound 104.

The Compound 103 (400 mg) was purified by Prep-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 90% B to 90% B in 25.5 min; Wave Length: 220/254 nm; RT1 (min): 13.02; RT2 (min): 17.49, the second peak is product) to afford the crude product. The crude product was purified by reversed-phase flash chromatography with the following conditions: column, C18 silica gel; mobile phase, MeCN in water (0.1% FA), 0% to 30% gradient in 20 min; detector, UV 254 nm. This resulted in Compound 103 (86.7 mg, 21.67%) as a yellow solid.

LC-MS-Compound 104: (ES, m/z): [M−HCOOH+H]⁺ 1028

H-NMR-Compound 104: (400 MHz, DMSO-d₆, δ ppm): 1.33-1.36 (m, 1H), 1.43-1.46 (m, 1H), 1.66-1.98 (m, 7H), 2.03-2.07 (m, 4H), 2.30-2.42 (m, 1H), 2.50-2.69 (m, 2H), 2.80-2.97 (m, 5H), 3.20-3.32 (m, 3H), 3.41-3.55 (m, 16H), 4.21-4.24 (m, 2H), 4.32-4.36 (d, 1H), 5.04-5.08 (d, 1H), 6.95-7.01 (m, 3H), 7.06-7.10 (m, 2H), 7.18-7.20 (d, 1H), 7.28-7.30 (m, 3H), 7.41-7.45 (m, 1H) 7.54-7.56 (m, 1H), 7.69-7.74 (m, 3H), 8.14 (s, 1H), 8.32 (s, 1H), 10.95 (s, 1H).

### Example 103. Compound 105

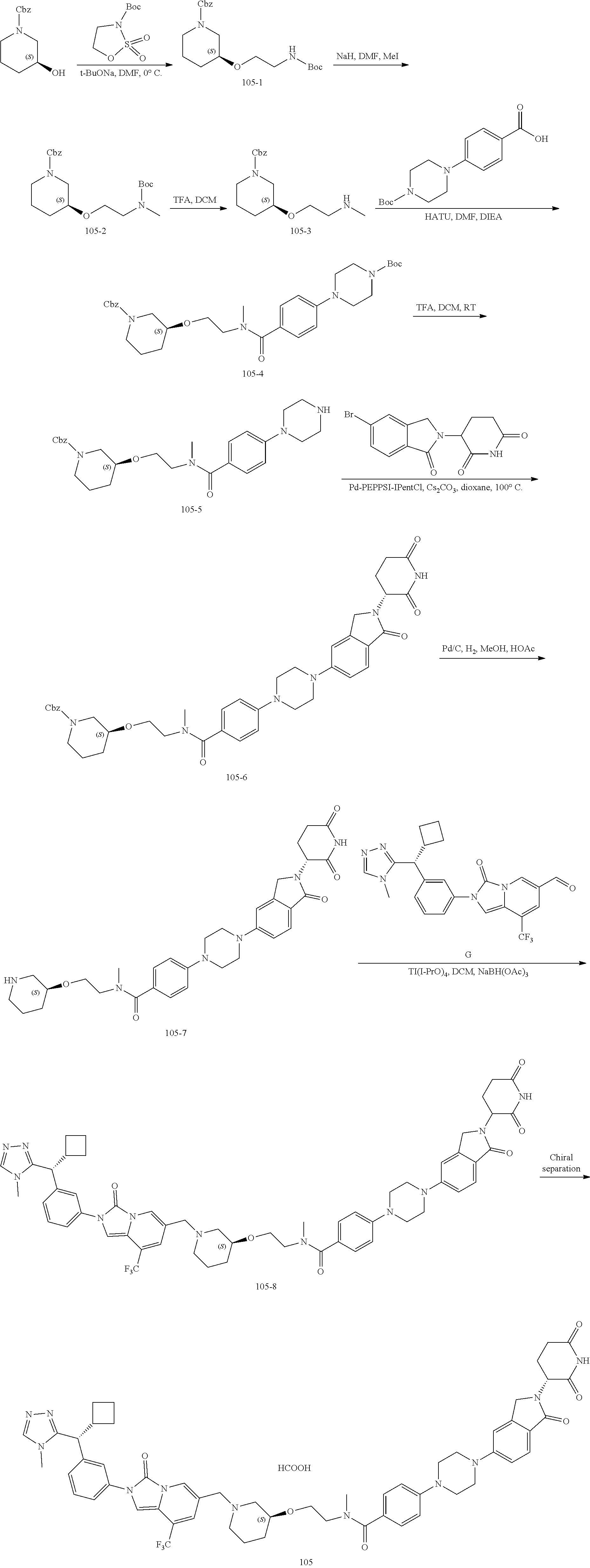

### Synthesis of Compound 105-1.

To a stirred mixture of benzyl (3S)-3-hydroxypiperidine-1-carboxylate (10 g, 42.502 mmol, 1 equiv) and tert-butyl 2,2-dioxo-1,2lambda6,3-oxathiazolidine-3-carboxylate (11.39 g, 51.002 mmol, 1.2 equiv) in DMF (100 mL) was added t-BuONa (8.17 g, 85.004 mmol, 2 equiv) in portions at 0° C. The resulting mixture was stirred overnight at room temperature. The mixture was neutralized to pH 7 with 4M HCl (aq). The aqueous layer was extracted with EtOAc (3×100 mL). The resulting mixture was concentrated under vacuum. The residue was purified by silica gel column chromatography, eluted with CH₂Cl₂/MeOH (100:1) to afford Compound 105-1 (15 g, 93.25%) as a white solid.

### Synthesis of Compound 105-2.

To a stirred mixture of Compound 105-1 (14.9 g, 39.369 mmol, 1 equiv) in DMF (200 mL) was added NaH (3.15 g, 78.738 mmol, 2 equiv, 60%) at 0° C. under nitrogen atmosphere. The resulting mixture was stirred for 1 h at room temperature under nitrogen atmosphere. To the above mixture was added MeI (6.71 g, 47.243 mmol, 1.2 equiv) at room temperature. The resulting mixture was stirred overnight at room temperature. The reaction was quenched with NH₄Cl (aq.) (600 mL) at room temperature. The aqueous layer was extracted with EtOAc (5×100 mL). The resulting mixture was concentrated under vacuum. The residue was purified by silica gel column chromatography, eluted with PE/EA (5:1) to afford Compound 105-2 (12 g, 77.66%) as a brown solid.

### Synthesis of Compound 105-3.

To a stirred solution of Compound 105-2 (11.9 g, 30.319 mmol, 1 equiv) and DCM (100 mL) was added TFA (33 mL) dropwise at room temperature. The resulting mixture was stirred for 1 h at room temperature. The resulting mixture was concentrated under vacuum. This resulted in Compound 105-3 (12 g, 97.47%) as a brown solid.

### Synthesis of Compound 105-4.

To a stirred mixture of 4-[4-(tert-butoxycarbonyl)piperazin-1-yl]benzoic acid (12.57 g, 41.043 mmol, 1 equiv), DIEA (21.22 g, 164.172 mmol, 4 equiv) and HATU (28.09 g, 73.877 mmol, 1.8 equiv) in DMF (130 mL) at room temperature. The resulting mixture was stirred for 1 h at room temperature. To the above mixture was added Compound 105-3 (12 g, 41.043 mmol, 1 equiv) at room temperature. The resulting mixture was stirred for additional 5 h at room temperature. The reaction was quenched with NH₄Cl (aq.) (400 mL) at room temperature. The aqueous layer was extracted with EtOAc (3×100 mL). The resulting mixture was concentrated under vacuum. The residue was purified by silica gel column chromatography, eluted with CH₂Cl₂/MeOH (100:1) to afford Compound 105-4 (7 g, 29.37%) as a brown solid.

### Synthesis of Compound 105-5.

To a stirred mixture of Compound 105-4 (7 g, 12.054 mmol, 1 equiv) in DCM (50 mL) was added TFA (15 mL) dropwise at room temperature. The resulting mixture was stirred for 1 h at room temperature. The resulting mixture was concentrated under vacuum. The residue was purified by reversed-phase flash chromatography with the following conditions: column, C18 silica gel; mobile phase, MeCN in water (10 mmol/L NH₄HCO₃), 10% to 50% gradient in 35 min; detector, UV 220 nm. This resulted in Compound 105-5 (2.75 g, 47.47%) as a brown solid.

### Synthesis of Compound 105-6.

To a stirred solution of Compound 105-5 (2.7 g, 5.618 mmol, 1 equiv) and 3-(5-bromo-1-oxo-3H-isoindol-2-yl)piperidine-2,6-dione (3.63 g, 11.236 mmol, 2 equiv) in dioxane (50 mL) was added Cs₂CO₃ (5.49 g, 16.854 mmol, 3 equiv) and Pd-PEPPSI-IPentCl 2-methylpyridine (o-picoline) (0.47 g, 0.562 mmol, 0.1 equiv) at room temperature under nitrogen atmosphere. The resulting mixture was stirred overnight at 100° C. under nitrogen atmosphere. The mixture was allowed to cool down to room temperature. The mixture was acidified to pH 6 with HOAc. The resulting mixture was concentrated under vacuum. The residue was purified by silica gel column chromatography, eluted with DCM (0.5% HOAc)/MeOH (20:1) to afford Compound 105-6 (1.2 g, 26.00%) as a light brown solid.

### Synthesis of Compound 105-7.

To a solution of Compound 105-6 (1.2 g, 1.660 mmol, 1 equiv) and HOAc (2 mL) in MeOH (50 mL) was added Pd/C (10%, 0.12 g) under nitrogen atmosphere in a 250 mL round-bottom flask. The mixture was hydrogenated at room temperature for 2 h under hydrogen atmosphere using a hydrogen balloon, filtered through a Celite pad and concentrated under reduced pressure. This resulted in Compound 105-7 (800 mg, 81.86%) as an off-white solid.

### Synthesis of Compound 105-8.

To a stirred solution of Compound 105-7 (780 mg, 1.325 mmol, 1 equiv) and Intermediate G (844.80 mg, 1.855 mmol, 1.4 equiv) in DCM (20 mL) was added Ti(Oi-Pr)₄ (753.13 mg, 2.650 mmol, 2 equiv) at room temperature. The resulting mixture was stirred overnight at room temperature. To the above mixture was added NaBH(OAc)₃ (561.61 mg, 2.650 mmol, 2 equiv) at room temperature. The resulting mixture was stirred for additional 4 h at room temperature. The reaction was quenched with 10% HOAc(aq.) (150 mL) at room temperature. The resulting mixture was filtered, the filter cake was washed with 10% HOAc(aq.) (20 mL). The aqueous layer was extracted with EtOAc (100 mL). The aqueous layer was concentrated under vacuum. The residue was purified by reversed-phase flash chromatography with the following conditions: column, C18 silica gel; mobile phase, MeCN in water (0.1% FA), 10% to 30% gradient in 30 min; detector, UV 254 nm. This resulted in Compound 105-8 (700 mg, 51.39%) as a yellow solid.

### Synthesis of Compound 105.

The Compound 105-8 (400 mg) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 90% B to 90% B in 25 min; Wave Length: 220/254 nm; RT1 (min): 6.59; RT2 (min): 15.18; the first peak is product) to afford the crude product. The crude product was purified by reversed-phase flash chromatography with the following conditions: column, C18 silica gel; mobile phase, MeCN in water (0.1% FA), 0% to 30% gradient in 20 min; detector, UV 254 nm. This resulted in Compound 105 (120.2 mg, 30.05%) as a yellow solid.

LC-MS-Compound 105: (ES, m/z): [M−HCOOH+H]⁺ 1028

H-NMR-Compound 105: (400 MHz, DMSO-d6, δ ppm): 1.33-1.36 (m, 1H), 1.43-1.46 (m, 1H), 1.66-1.98 (m, 7H), 2.03-2.07 (m, 4H), 2.30-2.42 (m, 1H), 2.51-2.57 (m, 2H), 2.80-2.97 (m, 5H), 3.15-3.32 (m, 3H), 3.35-3.55 (m, 16H), 4.21-4.24 (m, 2H), 4.32-4.36 (m, 1H), 5.05-5.08 (d, 1H), 6.94-7.01 (m, 2H), 7.01 (s, 1H), 7.08-7.10 (m, 2H), 7.18-7.20 (d, 1H), 7.30-7.32 (m, 3H), 7.41-7.45 (m, 1H) 7.53-7.56 (d, 1H), 7.68-7.74 (m, 3H), 8.13 (s, 1H), 8.31 (s, 1H), 10.95 (s, 1H).

### Example 104. Compound 106

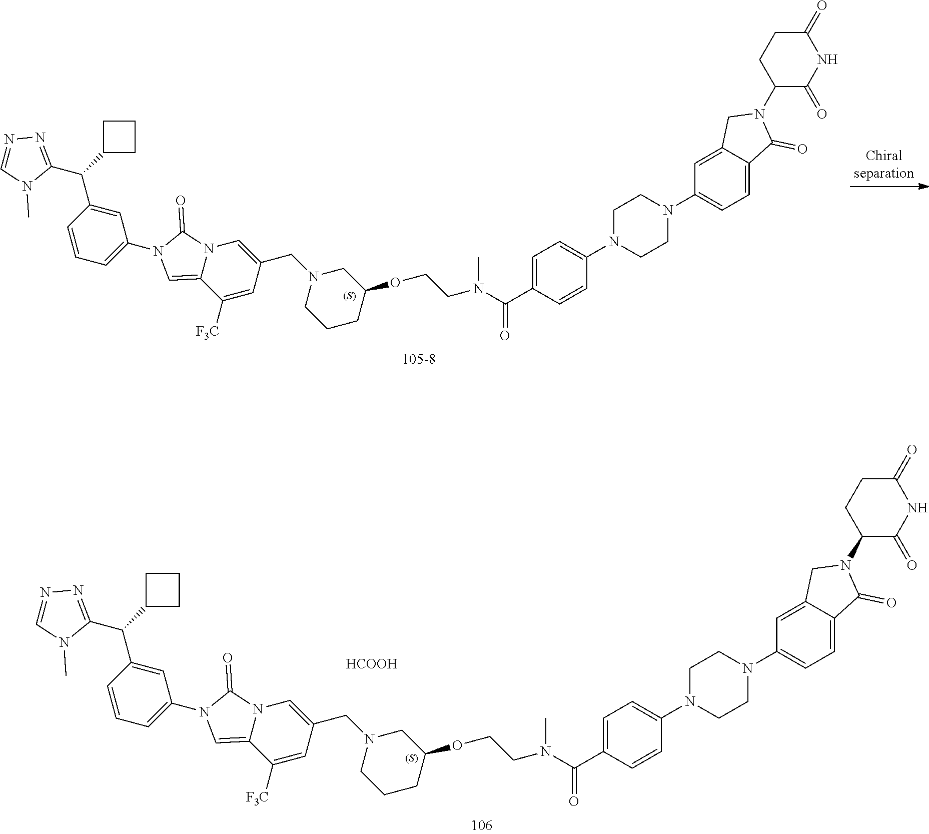

### Synthesis of Compound 106.

The Compound 105-8 (400 mg) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 90% B to 90% B in 25 min; Wave Length: 220/254 nm; RT1 (min): 6.59; RT2 (min): 15.18; the second peak is product) to afford the crude product. The crude product was purified by reversed-phase flash chromatography with the following conditions: column, C18 silica gel; mobile phase, MeCN in water (0.1% FA), 0% to 30% gradient in 20 min; detector, UV 254 nm. This resulted in Compound 105 (120.6 mg, 30.15%) as a yellow solid.

LC-MS-Compound 106: (ES, m/z): [M−HCOOH+H]⁺ 1028

H-NMR-Compound 106: (400 MHz, DMSO-d6, δ ppm): 1.33-1.36 (m, 1H), 1.43-1.46 (m, 1H), 1.66-1.98 (m, 7H), 2.03-2.07 (m, 4H), 2.30-2.42 (m, 1H), 2.51-2.57 (m, 2H), 2.80-2.97 (m, 5H), 3.15-3.32 (m, 3H), 3.35-3.55 (m, 16H), 4.21-4.24 (m, 2H), 4.32-4.36 (m, 1H), 5.05-5.08 (d, 1H), 6.94-7.01 (m, 3H), 7.08-7.20 (m, 3H), 7.30-7.32 (m, 3H), 7.41-7.45 (m, 1H) 7.53-7.56 (d, 1H), 7.68-7.74 (m, 3H), 8.13 (s, 1H), 8.31 (s, 1H), 10.95 (s, 1H).

### Example 105. Compound 107

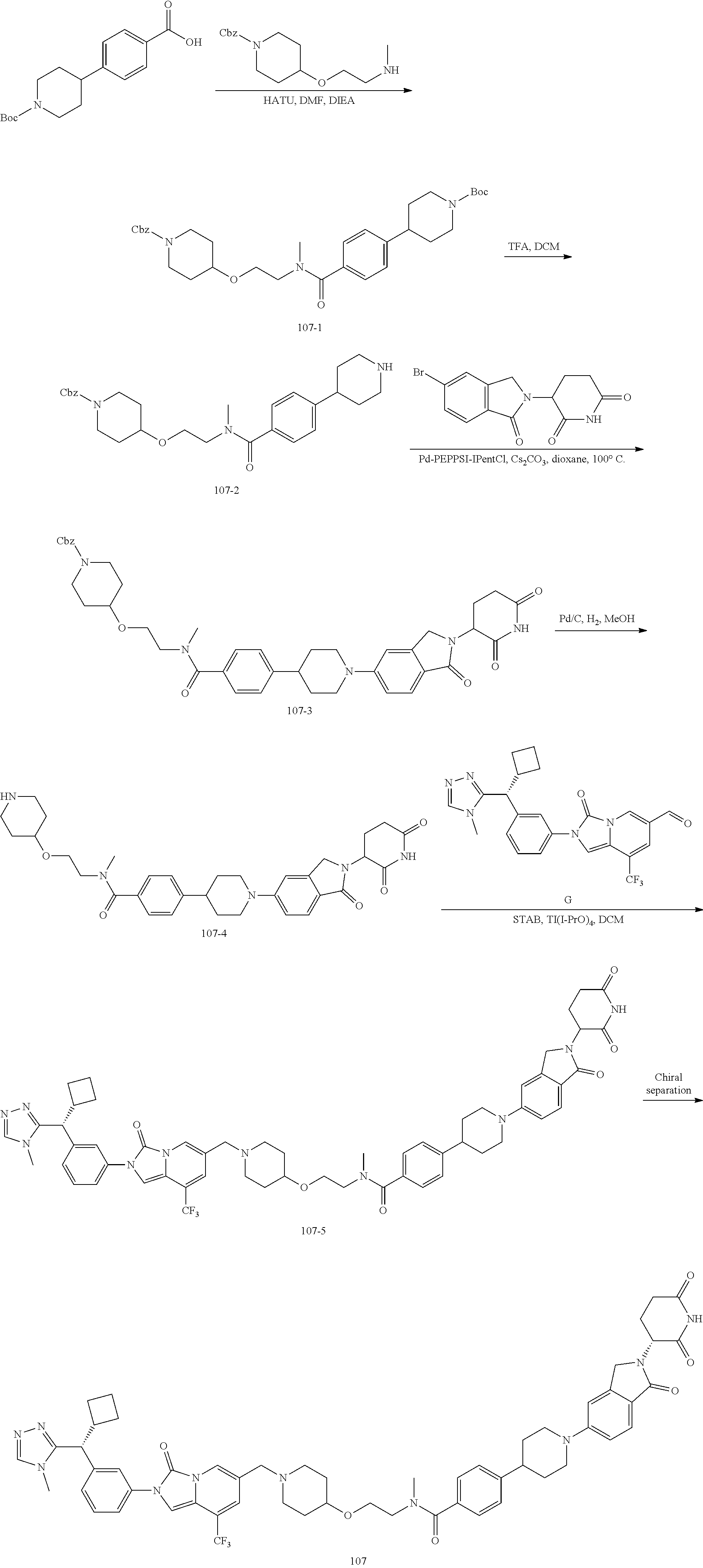

### Synthesis of Compound 107-1.

To a stirred solution of 4-[1-(tert-butoxycarbonyl) piperidin-4-yl] benzoic acid (10 g, 32.74 mmol, 1.0 equiv) and DIEA (8.4 g, 65.49 mmol, 2.0 equiv) in DMF (100 mL) were added HATU (18.6 g, 49.12 mmol, 1.5 equiv) at room temperature. The resulting mixture was stirred for 30 min at room temperature. To the above mixture was added benzyl 4-[2-(methylamino) ethoxy] piperidine-1-carboxylate (11.5 g, 39.29 mmol, 1.2 equiv) at room temperature. The resulting mixture was stirred for additional overnight at room temperature. The reaction was quenched by the addition of sat. NH₄Cl (aq.) (500 mL) at room temperature. The resulting mixture was extracted with EtOAc (3×300 mL). The combined organic layers were washed with water (3×300 mL), dried over anhydrous Na₂SO₄. After filtration, the filtrate was concentrated under reduced pressure. The residue was purified by silica gel column chromatography, eluted with PE/EA (5:1) to afford Compound 107-1 (5 g, 26%) as an off-white solid.

### Synthesis of Compound 107-2.

A solution of Compound 107-1 (5 g, 8.62 mmol, 1.0 equiv) and TFA (10 mL) in DCM (40 mL) was stirred for overnight at room temperature. The mixture was basified to pH 7 with NH₃ in MeOH. The residue was purified by silica gel column chromatography, eluted with CH₂Cl₂/MeOH (10:1) to afford Compound 107-2 (3.5 g, 85%) as a colorless oil.

### Synthesis of Compound 107-3.

To a stirred solution of Compound 107-2 (3.5 g, 7.29 mmol, 1.0 equiv) and 3-(5-bromo-1-oxo-3H-isoindol-2-yl) piperidine-2,6-dione (4.7 g, 14.59 mmol, 2.0 equiv) in 1,4-dioxane (35 mL) were added Cs₂CO₃ (7.1 g, 21.89 mmol, 3.0 equiv) and Pd-PEPPSI-IPentCl 2-methylpyridine (o-picoline) (875 g, 0.72 mmol, 0.1 equiv) at room temperature under nitrogen atmosphere. The resulting mixture was stirred for overnight at 100° C. under nitrogen atmosphere. The reaction was quenched by the addition of 10% AcOH (300 mL) at room temperature. The aqueous layer was extracted with MeOH:CH₂Cl₂ (1:10) (5×300 mL). The residue was purified by silica gel column chromatography, eluted with CH₂Cl₂/MeOH (20:1) to afford Compound 107-3 (600 mg, 11%) as an off-white solid.

### Synthesis of Compound 107-4.

To a solution of Compound 107-3 (600 mg, 0.83 mmol, 1.0 equiv) in 18 mL MeOH was added Pd/C (10%, 200 mg) under nitrogen atmosphere in a 50 mL round-bottom flask. The mixture was hydrogenated at room temperature for 2 h under hydrogen atmosphere using a hydrogen balloon, filtered through a Celite pad and concentrated under reduced pressure. This resulted in Compound 107-4 (400 mg, 82%) as a colorless oil.

### Synthesis of Compound 107-5.

To a stirred solution of Compound 107-4 (380 mg, 0.64 mmol, 1.0 equiv.) and Intermediate G (441 mg, 0.97 mmol, 1.5 equiv) in DCM (3.8 mL) was added Ti(Oi-Pr)₄ (735 mg, 2.58 mmol, 4.0 equiv) at room temperature. The resulting mixture was stirred for overnight at room temperature. To the above mixture was added STAB (411 mg, 1.94 mmol, 3.0 equiv) at room temperature. The resulting mixture was stirred for additional 2 h at room temperature. The reaction was quenched by the addition of 10% AcOH (100 mL) at room temperature. The aqueous layer was extracted with EtOAc (2×60 mL). The aqueous phase was concentrated under reduced pressure. The residue was purified by reversed-phase flash chromatography with the following conditions: column, C18 silica gel; mobile phase, MeCN in water (0.1% FA), 10% to 50% gradient in 30 min; detector, UV 254 nm. The crude product (200 mg) was purified by Prep-HPLC with the following conditions (Column: Xselect CSH C18 OBD Column 30*150 mm 5 μm, n; Mobile Phase A: Water (0.1% FA) Water (0.1% FA), Mobile Phase B: ACN; Flow rate: 60 mL/min; Gradient: 22% B to 34% B in 8 min, 34% B; Wave Length: 254; 220 nm; RT1 (min): 7.78) to afford Compound 107-5 (100 mg, 15%) as a yellow solid.

### Synthesis of Compound 107.

The Compound 107-5 (100 mg) was purified by Prep-CHIRAL-HPLC with the following conditions Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 90% B to 90% B in 20 min; Wave Length: 220/254 nm; RT1 (min): 9.02; RT2 (min): 15.58; Sample Solvent: DCM:ACN=1:1—HPLC; Injection Volume: 0.7 mL; Number of Runs: 4. The first peak was the product. This resulted in Compound 107 (34 mg, 34%) as a yellow solid.

LC-MS-Compound 107: (ES, m/z): [M+H]⁺ 1027

H-NMR-Compound 107: (400 MHz, CD₃OD, δ ppm): 1.31-1.35 (m, 1H), 1.59-1.99 (m, 14H), 2.10-2.25 (m, 2H), 2.37-2.52 (m, 3H), 2.71-2.87 (m, 5H), 2.88-3.10 (m, 5H), 3.33-3.54 (m, 8H), 3.72-3.81 (m, 2H), 4.03-4.09 (m, 2H), 4.27-4.30 (d, 1H), 4.41-4.43 (m, 2H), 5.11-5.18 (m, 1H), 7.11-7.14 (m, 4H), 7.26-7.28 (d, 1H), 7.39-7.41 (m, 4H), 7.47-7.51 (m, 1H), 7.64-7.66 (m, 3H), 7.71 (s, 1H), 8.37 (s, 1H).

### Example 106. Compound 108

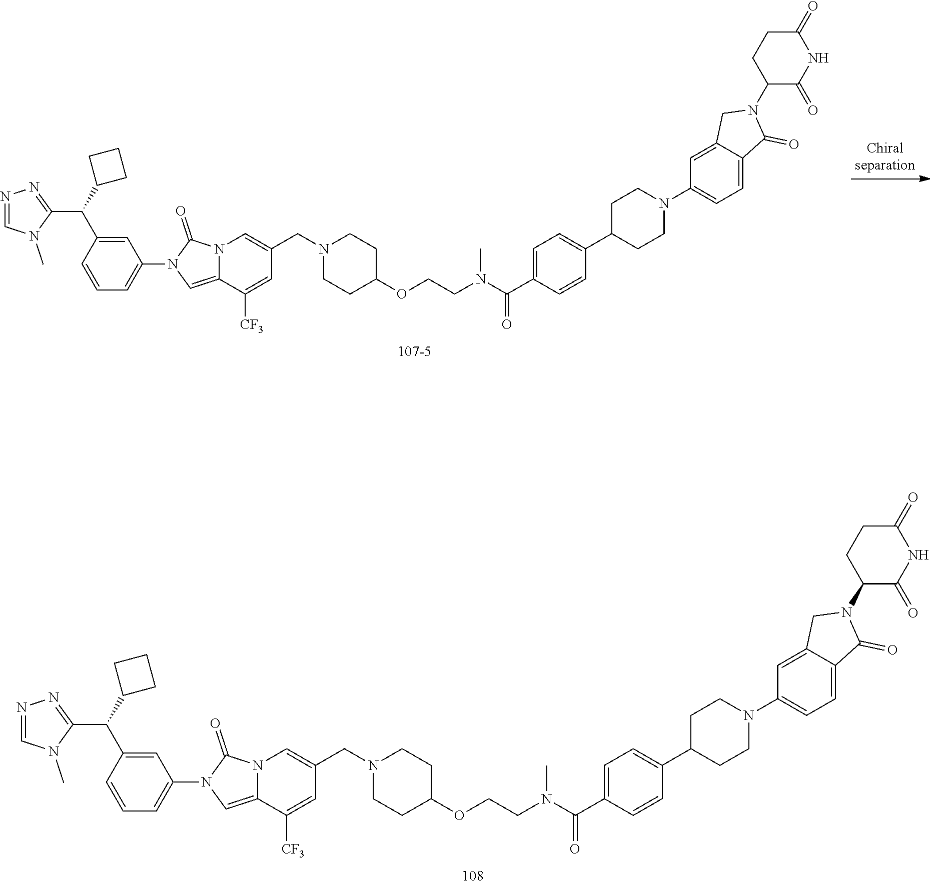 [SMILES: CN(CCOC1CCN(Cc2cc(C(F)(F)F)c3cn(-c4cccc([C@H](c5nncn5C)C5CCC5)c4)c(=O)n3c2)CC1)C(=O)c1ccc(C2CCN(c3ccc4c(c3)CN(C3CCC(=O)NC3=O)C4=O)CC2)cc1 ; CN(CCOC1CCN(Cc2cc(C(F)(F)F)c3cn(-c4cccc([C@H](c5nncn5C)C5CCC5)c4)c(=O)n3c2)CC1)C(=O)c1ccc(C2CCN(c3ccc4c(c3)CN([C@H]3CCC(=O)NC3=O)C4=O)CC2)cc1]

### Synthesis of Compound 108.

The Compound 107-5 (100 mg) was purified by Prep-CHIRAL-HPLC with the following conditions Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 90% B to 90% B in 20 min; Wave Length: 220/254 nm; RT1 (min): 9.02; RT2 (min): 15.58; Sample Solvent: DCM:ACN=1:1—HPLC; Injection Volume: 0.7 mL; Number of Runs: 4. The second peak was the product. This resulted in Compound 108 (35 mg, 35%) as a yellow solid.

LC-MS-Compound 108: (ES, m/z): [M+H]⁺ 1027

H-NMR-Compound 108: (400 MHz, CD₃OD, δ ppm): 1.31-1.35 (m, 1H), 1.59-1.99 (m, 14H), 2.10-2.25 (m, 2H), 2.37-2.52 (m, 3H), 2.71-2.87 (m, 5H), 2.88-3.10 (m, 5H), 3.33-3.54 (m, 8H), 3.72-3.81 (m, 2H), 4.03-4.09 (m, 2H), 4.27-4.30 (d, 1H), 4.41-4.43 (m, 2H), 5.11-5.18 (m, 1H), 7.11-7.14 (m, 4H), 7.26-7.28 (d, 1H), 7.39-7.41 (m, 4H), 7.47-7.51 (m, 1H), 7.64-7.66 (m, 3H), 7.71 (s, 1H), 8.37 (s, 1H).

### Example 107. Compound 109

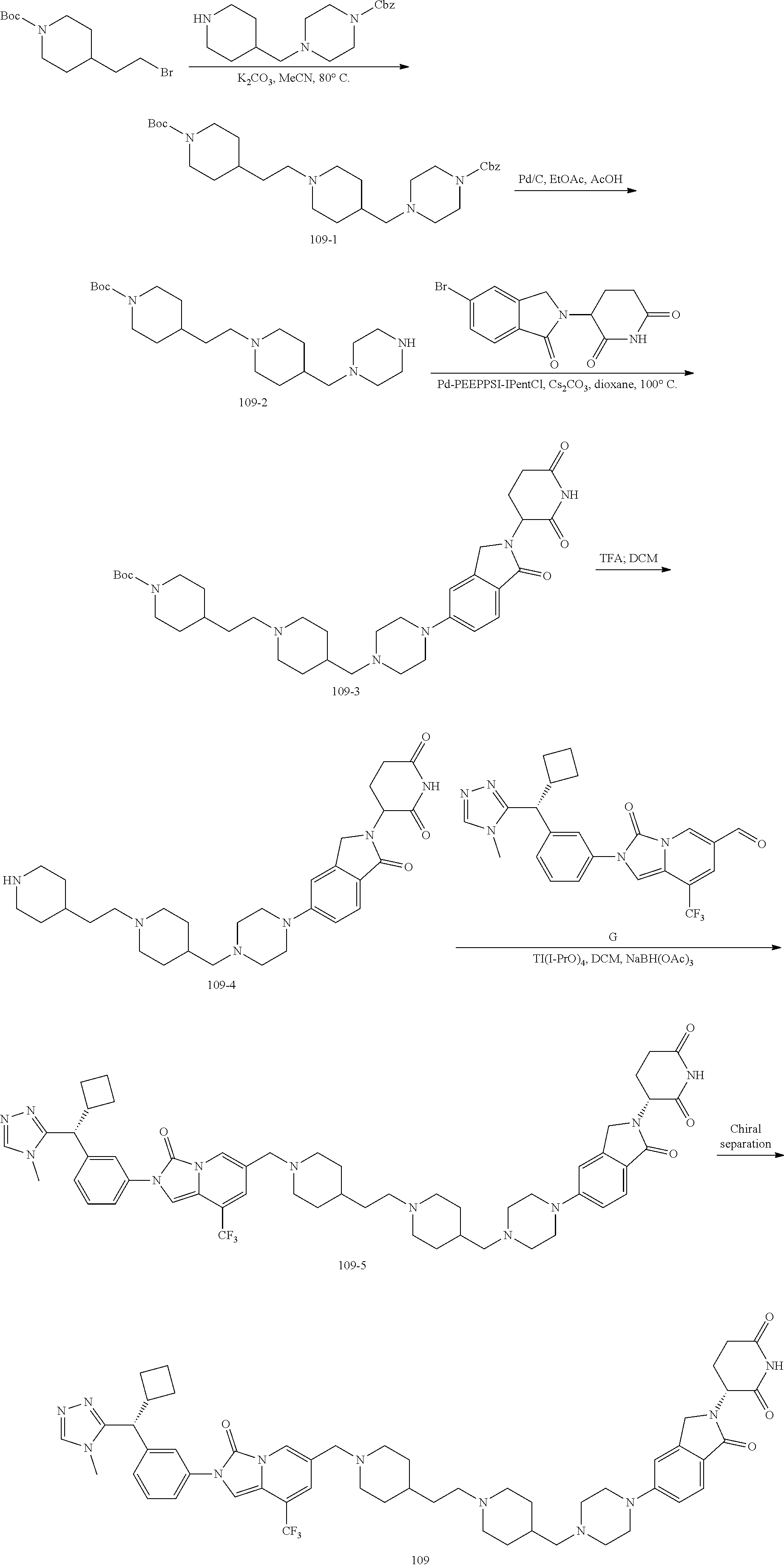

### Synthesis of Compound 109-1.

To a stirred solution of tert-butyl 4-(2-bromomethyl)piperidine-1-carboxylate (5 g, 17.111 mmol, 1.00 equiv) and benzyl 4-(piperidin-4-ylmethyl)piperazine-1-carboxylate (5.43 g, 17.111 mmol, 1.00 equiv) in MeCN (50 mL) was added K₂CO₃ (4.73 g, 34.222 mmol, 2 equiv). The resulting mixture was stirred for overnight at room temperature under nitrogen atmosphere. The reaction was quenched with sat. NH₄Cl (aq.) (100 mL) at room temperature. The resulting mixture was extracted with CH₂Cl₂/MeOH (10:1) (3×50 mL). The combined organic layers were concentrated under reduced pressure. The residue was purified by silica gel column chromatography, eluted with CH₂Cl₂/MeOH (30:1) to afford Compound 109-1 (5 g, 53.61%) as a yellow solid.

### Synthesis of Compound 109-2.

To a solution of Compound 109-1 (5 g, 9.456 mmol, 1 equiv) in EtOAc (150 mL) was added Pd/C (10%, 1 g) under nitrogen atmosphere in a 500 mL round-bottom flask. The mixture was hydrogenated at room temperature for 1 h under hydrogen atmosphere using a hydrogen balloon. The resulting mixture was concentrated under vacuum. This resulted in Compound 109-2 as a off-white solid.

### Synthesis of Compound 109-3.

To a stirred mixture of Compound 109-2 (2.5 g, 6.335 mmol, 1 equiv) and 3-(5-bromo-1-oxo-3H-isoindol-2-yl)piperidine-2,6-dione (2.46 g, 7.602 mmol, 1.2 equiv) in dioxane (60 mL) were added Cs₂CO₃ (6.19 g, 19.005 mmol, 3 equiv) and Pd-PEPPSI-IPentCl 2-methylpyridine (o-picoline (0.53 g, 0.634 mmol, 0.1 equiv) under nitrogen atmosphere. The resulting mixture was stirred for overnight at 100° C. under nitrogen atmosphere. The resulting mixture was diluted with water/HOAc=10/1 (200 mL). The resulting mixture was extracted with CH₂Cl₂/MeOH=10/1 (4×150 mL). The combined organic layers were concentrated under reduced pressure. The residue was purified by silica gel column chromatography, eluted with CH₂Cl₂/MeOH (8:1) to afford Compound 109-3 (2 g, 49.57%) as a brown solid.

### Synthesis of Compound 109-4.

To a stirred solution of Compound 109-3 (2.1 g, 3.298 mmol, 1 equiv) in DCM (20 mL) was added TFA (5 mL). The resulting mixture was stirred for 3 h at room temperature. The resulting mixture was concentrated under reduced pressure. This resulted in Compound 109-4 (1.76 g, 99.44%) as a brown solid.

### Synthesis of Compound 109-5.

To a stirred solution of Compound 109-4 (850 mg, 1.584 mmol, 1 equiv) and Intermediate G (721.28 mg, 1.584 mmol, 1 equiv) in DCM (15 mL) was added Ti(Oi-Pr)₄ (1800.46 mg, 6.336 mmol, 4 equiv). The resulting mixture was stirred for overnight at room temperature. To the above mixture was added NaBH(OAc)₃ (1006.94 mg, 4.752 mmol, 3 equiv). The resulting mixture was stirred for additional 4 h at room temperature. The reaction was quenched by the addition of Water/HOAc (100 mL). The resulting mixture was extracted with CH₂Cl₂/MeOH=8/1 (3×100 mL). The combined organic layers were concentrated under reduced pressure. The residue was purified by reverse flash chromatography with the following conditions: column, C18 silica gel; mobile phase, MeCN in Water (0.1% FA), 5% to 50% gradient in 30 min; detector, UV 254 nm. This resulted in Compound 109-5 (820 mg, 53.04%) as a yellow solid.

### Synthesis of Compound 109.

Compound 109-5 (400 mg) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 60% B to 60% B in 14.5 min; Wave Length: 220/254 nm; RT1 (min): 6.80; RT2 (min): 10.79; the first peak was product) to afford Compound 109 (123 mg, 30.75%) as a yellow solid.

LCMS-Compound 109: (ES, m/z): [M+H]⁺ 977

NMR-Compound 109: (400 MHz, DMSO, δ ppm): δ1.18-1.48 (m, 8H), 1.62-1.81 (m, 11H), 194-1.98 (m, 3H), 2.02-2.11 (m, 1H), 2.15-2.21 (m, 2H), 2.31-2.40 (m, 2H), 2.41-2.43 (m, 3H), 2.52-2.65 (m, 1H), 2.68-2.80 (m, 2H), 2.82-2.93 (m, 3H), 3.12-3.27 (m, 4H), 3.36-3.48 (m, 8H), 4.18-4.34 (m, 3H), 5.04-5.08 (m, 1H), 7.00-7.06 (m, 3H), 7.19-7.21 (m, 1H), 7.29-7.35 (m, 2H), 7.42-7.46 (t, 1H), 7.51-7.53 (m, 1H), 7.65-7.73 (m, 2H), 8.33 (s, 1H), 10.95 (s, 1H).

### Example 108. Compound 110

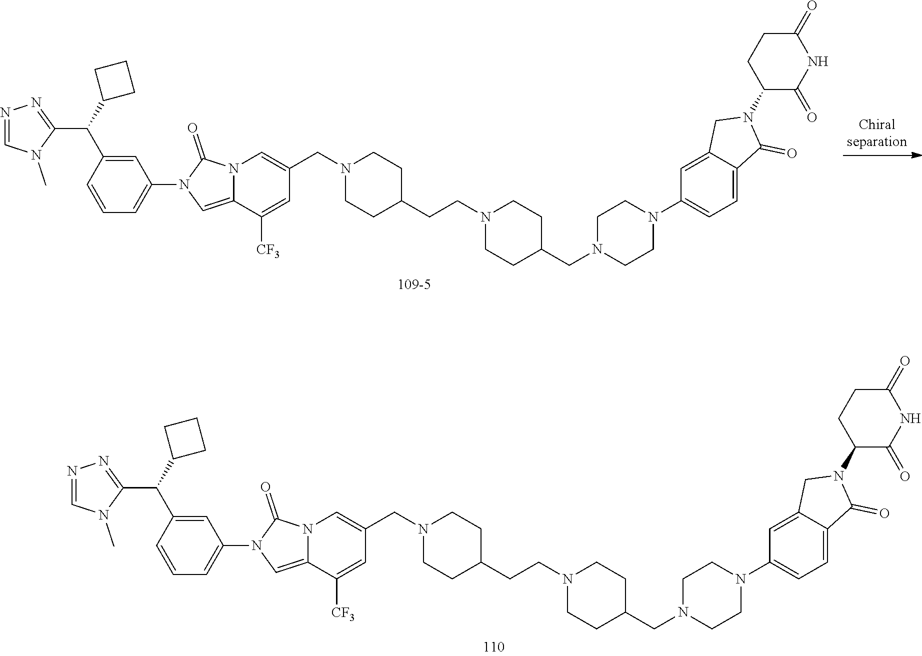 [SMILES: Cn1cnnc1[C@@H](c1cccc(-n2cc3c(C(F)(F)F)cc(CN4CCC(CCN5CCC(CN6CCN(c7ccc8c(c7)CN([C@@H]7CCC(=O)NC7=O)C8=O)CC6)CC5)CC4)cn3c2=O)c1)C1CCC1 ; Cn1cnnc1[C@@H](c1cccc(-n2cc3c(C(F)(F)F)cc(CN4CCC(CCN5CCC(CN6CCN(c7ccc8c(c7)CN([C@H]7CCC(=O)NC7=O)C8=O)CC6)CC5)CC4)cn3c2=O)c1)C1CCC1]

### Synthesis of Compound 110.

Compound 109-5 (400 mg) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 60% B to 60% B in 14.5 min; Wave Length: 220/254 nm; RT1 (min): 6.80; RT2 (min): 10.79; the second peak was product) to afford Compound 110 (83.4 mg, 20.85%) as a yellow solid.

LCMS-Compound 110: (ES, m/z): [M+H]⁺ 977

NMR-Compound 110: (400 MHz, DMSO, δ ppm): δ1.08-1.15 (m, 4H), 1.16-1.24 (m, 1H), 1.31-1.39 (m, 2H), 1.43-1.52 (m, 1H), 1.56-1.72 (m, 5H), 1.74-1.81 (m, 4H), 1.85-1.96 (m, 5H), 2.11-2.16 (m, 2H), 2.30-2.39 (m, 3H), 2.42-2.46 (m, 4H), 2.55-2.64 (m, 1H), 2.68-2.80 (m, 5H), 3.12-3.27 (m, 8H), 3.36-3.48 (m, 4H), 4.18-4.34 (m, 3H), 5.04-5.08 (m, 1H), 7.00-7.06 (m, 3H), 7.19-7.21 (m, 1H), 7.31 (s, 1H), 7.42-7.46 (t, 1H), 7.51-7.53 (m, 1H), 7.64-7.73 (m, 3H), 8.33 (s, 1H), 10.95 (s, 1H).

### Example 109. Compound 111

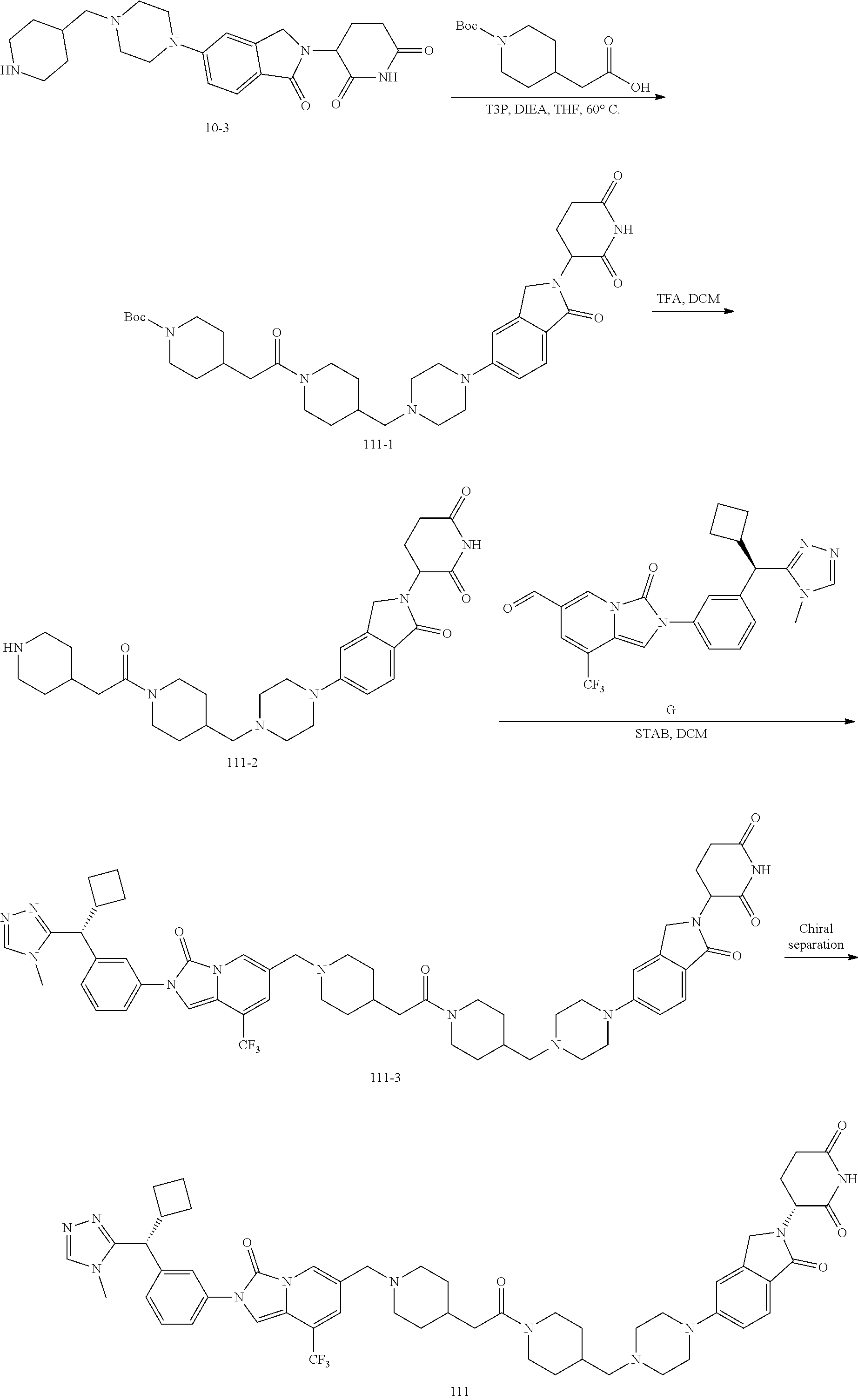

### Synthesis of Compound 111-1.

To a stirred solution of Compound 10-3 (3 g, 7.05 mmol, 1.0 equiv) and [1-(tert-butoxycarbonyl) piperidin-4-yl] acetic acid (2.1 g, 8.46 mmol, 1.2 equiv) in THF (30 mL) were added DIEA (1.8 g, 14.10 mmol, 2.0 equiv) and T3P (3.4 g, 10.57 mmol, 1.5 equiv) at room temperature. The resulting mixture was stirred for 3 h at 60° C. The reaction was quenched by the addition of sat. NH₄Cl (aq.) (100 mL) at room temperature. The resulting mixture was extracted with EtOAc (3×100 mL). The combined organic layers were washed with water (3×100 mL), dried over anhydrous Na₂SO₄. After filtration, the filtrate was concentrated under reduced pressure. The residue was purified by silica gel column chromatography, eluted with PE/EA (1:1) to afford Compound 111-1 (1.4 g, 30%) as an off-white solid.

### Synthesis of Compound 111-2.

A solution of Compound 111-1 (1.4 g, 2.15 mmol, 1.0 equiv) and TFA (4 mL) in DCM (12 mL) was stirred for overnight at room temperature. The mixture was basified to pH 7 with NH₃ in MeOH. The resulting mixture was concentrated under reduced pressure. The residue was purified by Prep-TLC (CH₂Cl₂/MeOH=10:1) to afford Compound 111-2 (900 mg, 76%) as an off-white solid.

### Synthesis of Compound 111-3.

To a stirred solution of Compound 111-2 (900 mg, 1.63 mmol, 1.0 equiv) and Intermediate G (1116 mg, 2.45 mmol, 1.5 equiv) in DCM (9 mL) was added Ti(Oi-Pr)₄ (1857 mg, 6.53 mmol, 4.0 equiv) at room temperature. The resulting mixture was stirred for overnight at room temperature. To the above mixture was added STAB (1039 mg, 4.90 mmol, 3.0 equiv) at room temperature. The resulting mixture was stirred for additional 2 h at room temperature. The reaction was quenched by the addition of 10% AcOH (100 mL) at room temperature. The aqueous layer was extracted with EtOAc (2×100 mL). The aqueous phase was concentrated under reduced pressure. The residue was purified by reversed-phase flash chromatography with the following conditions: column, C18 silica gel; mobile phase, MeCN in water (0.1% FA), 10% to 50% gradient in 30 min; detector, UV 254 nm. The crude product (600 mg) was purified by Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 12% B to 24% B in 7 min, 24% B; Wave Length: 254; 220 nm; RT1 (min): 6.5) to afford Compound 111-3 (260 mg, 16%) as a yellow solid.

### Synthesis of Compound 111.

The Compound 111-3 (260 mg) was purified by Prep-CHIRAL-HPLC with the following conditions Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 90% B to 90% B in 17 min; Wave Length: 220/254 min; RT1 (min): 7.96; RT2 (min): 12.73; Sample Solvent: DCM:ACN=1:1—HPLC; Injection Volume: 0.6 mL; Number of Runs: 8. The first peak was the product. This resulted in Compound 111 (91 mg, 35%) as a yellow solid.

LC-MS-Compound 111: (ES, m/z): [M+H]⁺ 990

H-NMR-Compound 111: (400 MHz, DMSO-d6, δ ppm): 0.78.91 (m, 2H), 1.21-1.24 (m, 3H), 1.73-1.80 (m, 12H), 1.94-2.09 (m, 4H), 2.18-2.23 (m, 4H), 2.35-2.38 (m, 2H), 2.50-2.55 (m, 2H), 2.87-2.97 (m, 4H), 3.21-3.37 (m, 8H), 3.42 (s, 3H), 3.85-3.90 (m, 1H), 4.17-4.27 (m, 3H), 4.30-4.34 (m, 1H), 5.02-5.07 (m, 1H), 7.04-7.06 (m, 3H), 7.19-7.21 (d, 1H), 7.32 (s, 1H), 7.42-7.46 (m, 1H), 7.50-7.53 (d, 1H), 7.68-7.77 (m, 3H), 8.32 (s, 1H), 10.95 (s, 1H).

### Example 110. Compound 112

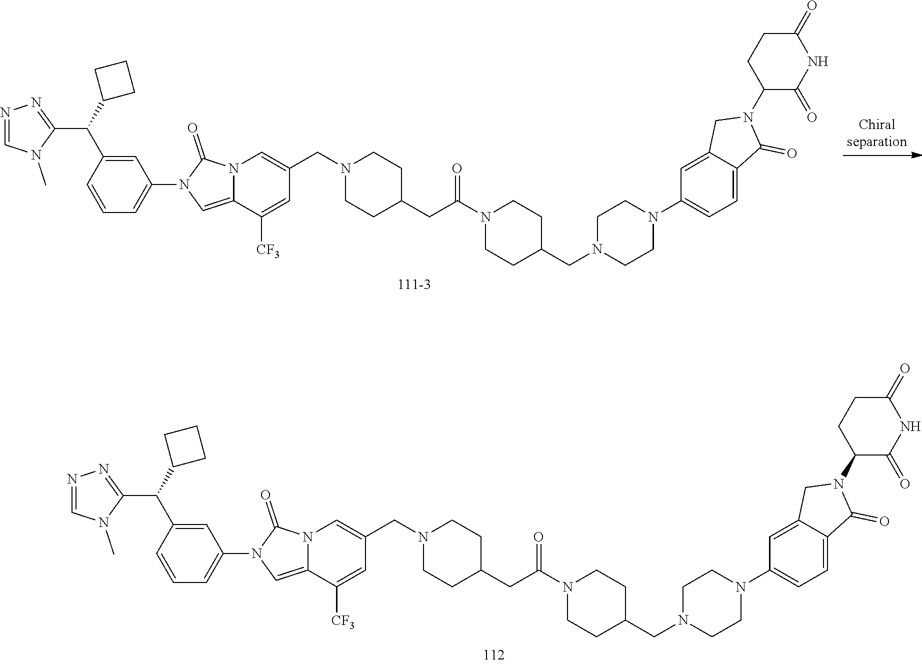 [SMILES: Cn1cnnc1[C@@H](c1cccc(-n2cc3c(C(F)(F)F)cc(CN4CCC(CC(=O)N5CCC(CN6CCN(c7ccc8c(c7)CN(C7CCC(=O)NC7=O)C8=O)CC6)CC5)CC4)cn3c2=O)c1)C1CCC1 ; Cn1cnnc1[C@@H](c1cccc(-n2cc3c(C(F)(F)F)cc(CN4CCC(CC(=O)N5CCC(CN6CCN(c7ccc8c(c7)CN([C@H]7CCC(=O)NC7=O)C8=O)CC6)CC5)CC4)cn3c2=O)c1)C1CCC1]

### Synthesis of Compound 112.

The Compound 111-3 (260 mg) was purified by Prep-CHIRAL-HPLC with the following conditions Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 90% B to 90% B in 17 min; Wave Length: 220/254 nm; RT1 (min): 7.96; RT2 (min): 12.73; Sample Solvent: DCM:ACN=1:1—HPLC; Injection Volume: 0.6 mL; Number of Runs: 8. The second peak was the product. This resulted in Compound 112 (74 mg, 28%) as a yellow solid.

LC-MS-Compound 112: (ES, m/z): [M+H]⁺ 990

H-NMR-Compound 112: (400 MHz, DMSO-d6, δ ppm): 0.78.91 (m, 2H), 1.21-1.24 (m, 3H), 1.73-1.80 (m, 12H), 1.94-2.09 (m, 4H), 2.18-2.23 (m, 4H), 2.35-2.38 (m, 2H), 2.50-2.55 (m, 2H), 2.87-2.97 (m, 4H), 3.21-3.37 (m, 8H), 3.42 (s, 3H), 3.85-3.90 (m, 1H), 4.17-4.27 (m, 3H), 4.30-4.34 (m, 1H), 5.02-5.07 (m, 1H), 7.04-7.06 (m, 3H), 7.19-7.21 (d, 1H), 7.32 (s, 1H), 7.42-7.46 (m, 1H), 7.50-7.53 (d, 1H), 7.68-7.77 (m, 3H), 8.32 (s, 1H), 10.95 (s, 1H).

### Example 111. Compound 113

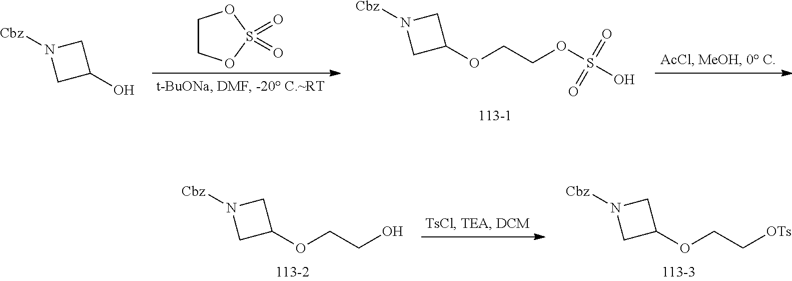 [SMILES: O=C(OCc1ccccc1)N1CC(O)C1 ; O=S1(=O)OCCO1 ; O=C(OCc1ccccc1)N1CC(OCCOS(=O)(=O)O)C1 ; O=C(OCc1ccccc1)N1CC(OCCO)C1 ; Cc1ccc(S(=O)(=O)OCCOC2CN(C(=O)OCc3ccccc3)C2)cc1]

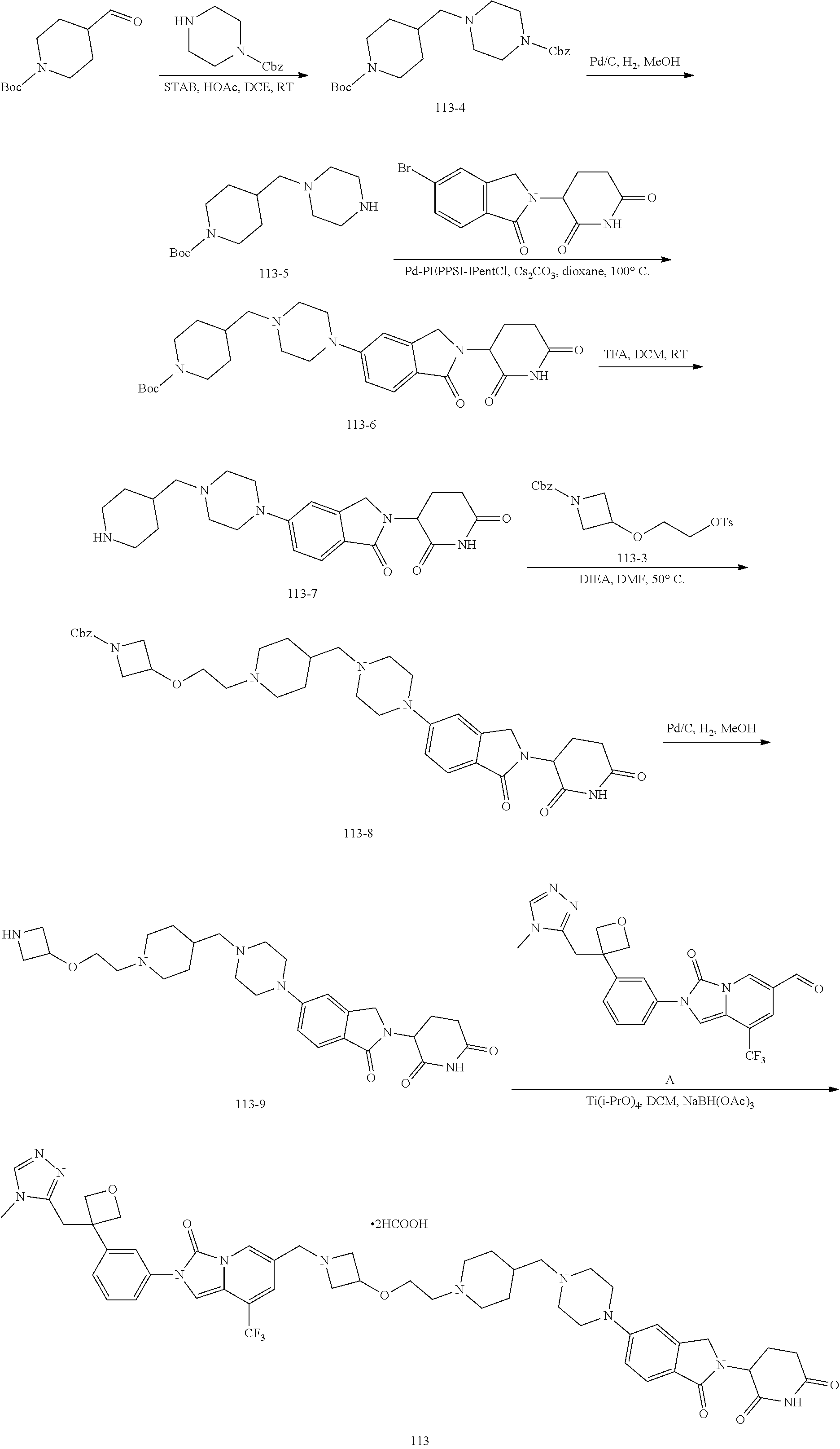

### Synthesis of Compound 113-1.

To a stirred solution of benzyl 3-hydroxyazetidine-1-carboxylate (25 g, 120.639 mmol, 1 equiv) and 1,3,2lambda6-dioxathiolane-2,2-dione (37.43 g, 301.597 mmol, 2.5 equiv) in DMF (250 mL) was added sodium 2-methylpropan-2-olate (28.99 g, 301.597 mmol, 2.5 equiv) at −20° C. under nitrogen atmosphere. The resulting mixture was stirred overnight at room temperature under nitrogen atmosphere. The reaction was quenched with NH₄Cl(aq.) at room temperature. The aqueous layer was extracted with DCM (3×200 mL). The aqueous layer was concentrated under reduced pressure. This resulted in Compound 113-1 (30 g, 67.55%) as a yellow oil. The crude product was used in the next step directly without further purification.

### Synthesis of Compound 113-2.

To a stirred solution of Compound 113-1 (25 g, 75.451 mmol, 1 equiv) in MeOH (250 mL, 24.699 mmol) was added AcCl (17.77 g, 226.353 mmol, 3 equiv) dropwise at 0° C. under. The resulting mixture was stirred overnight at room temperature. The mixture was neutralized with saturated NaHCO₃ (aq.). The aqueous layer was extracted with MTBE (3×150 mL). The aqueous layer was concentrated under reduced pressure. The crude product was purified by HP-FLASH with the following conditions (Mobile Phase A: Water (0.1% FA), Mobile Phase B: ACN; Flow rate: 60 mL/min; Gradient: 10% B to 30% B in 20 min, Wave Length: 220 nm) to afford Compound 13-2 (2.1 g, 9.97%) as a light yellow oil.

### Synthesis of Compound 113-3.

To a stirred solution of Compound 113-2 (2.1 g, 8.357 mmol, 1 equiv) and TEA (2.11 g, 20.892 mmol, 2.5 equiv) in DCM (30 mL) was added TsCl (3.98 g, 20.892 mmol, 2.5 equiv) at room temperature. The resulting mixture was stirred overnight at room temperature. The reaction was quenched with water (80 mL) at room temperature. The aqueous layer was extracted with DCM (3×50 mL). The resulting mixture was concentrated under reduced pressure. The residue was purified by silica gel column chromatography, eluted with DCM/MeOH (50:1) to afford Compound 113-3 (3.4 g, 95.32%) as a light yellow oil.

### Synthesis of Compound 113-4.

To a stirred solution of tert-butyl 4-formylpiperidine-1-carboxylate (100 g, 468.874 mmol, 1 equiv) and benzyl piperazine-1-carboxylate (103.28 g, 468.874 mmol, 1 equiv) in DCE (500 mL) was added STAB (198.75 g, 937.748 mmol, 2 equiv) and HOAc (2.82 g, 46.887 mmol, 0.1 equiv) at room temperature. The resulting mixture was stirred overnight at room temperature. The reaction was quenched with NH₄Cl (aq.) at room temperature. The aqueous layer was extracted with DCM (2×800 mL). The resulting mixture was concentrated under vacuum. The residue was purified by silica gel column chromatography, eluted with DCM to afford Compound 113-4 (170 g, 82.49%) as an off-white solid.

### Synthesis of Compound 113-5.

To a solution of Compound 113-4 (90 g, 215.543 mmol, 1 equiv) in MeOH (300 mL) was added Pd/C (9 g, 10%) under nitrogen atmosphere in a 1000 mL round-bottom flask. The mixture was hydrogenated at room temperature for overnight under hydrogen atmosphere using a hydrogen balloon, filtered through a Celite pad and concentrated under reduced pressure. This resulted in Compound 113-5 (60 g, 88.40%) as an off-white solid.

### Synthesis of Compound 113-6.

To a stirred solution of Compound 113-5 (6 g, 21.170 mmol, 1 equiv) and 3-(5-bromo-1-oxo-3H-isoindol-2-yl)piperidine-2,6-dione (13.68 g, 42.340 mmol, 2 equiv) in dioxane (200 mL) was added Cs₂CO₃ (20.69 g, 63.510 mmol, 3 equiv) and Pd-PEPPSI-IPentCl 2-methylpyridine (o-picoline) (1.78 g, 2.117 mmol, 0.1 equiv) at room temperature under nitrogen atmosphere. The resulting mixture was stirred overnight at 100° C. under nitrogen atmosphere. The mixture was acidified to pH 6 with HOAc. The resulting mixture was concentrated under vacuum. The residue was purified by silica gel column chromatography, eluted with DCM/MeOH (30:1) to afford Compound 113-6 (5 g, 35.94%) as a light brown solid.

### Synthesis of Compound 113-7.

A solution of Compound 113-6 (5 g, 9.512 mmol, 1 equiv) and TFA (5 g) in DCM (20 mL) was stirred overnight at room temperature. The resulting mixture was concentrated under vacuum. The residue was purified by reverse flash chromatography with the following conditions: column, C18 silica gel; mobile phase, MeCN in water (0.1% FA), 0% to 40% gradient in 20 min; detector, UV 220 nm. This resulted in Compound 113-7 (4 g, 84.00%) as a light brown solid.

### Synthesis of Compound 113-8.

To a stirred solution of Compound 113-7 (3.15 g, 7.399 mmol, 1.00 equiv) and Compound 113-3 (3 g, 7.399 mmol, 1.00 equiv) in DMF (30 mL) was added DIEA (2.87 g, 22.197 mmol, 3 equiv) at room temperature. The resulting mixture was stirred overnight at 50° C. The mixture was allowed to cool down to room temperature. The residue was purified by reverse flash chromatography with the following conditions: column, C18 silica gel; mobile phase, MeCN in water (0.1% FA), 0% to 50% gradient in 20 min; detector, UV 220 nm. This resulted in Compound 113-8 (2.5 g, 46.67%) as a light brown solid.

### Synthesis of Compound 113-9.

To a solution of Compound 113-8 (2.5 g, 3.795 mmol, 1 equiv) in MeOH (75 mL) was added Pd/C (0.25 g, 2.349 mmol, 0.62 equiv) under nitrogen atmosphere in a 250 mL round-bottom flask. The mixture was hydrogenated at room temperature for 3 h under hydrogen atmosphere using a hydrogen balloon, filtered through a Celite pad and concentrated under reduced pressure. This resulted in Compound 113-9 (1.5 g, 67.81%) as an off-white solid.

### Synthesis of Compound 113.

To a stirred solution of Compound 113-9 (700 mg, 1.334 mmol, 1 equiv) and Intermediate A (610.27 mg, 1.334 mmol, 1 equiv) in DCM (20 ml) was added Ti(Oi-Pr)₄ (1137.60 mg, 4.002 mmol, 3 equiv) at room temperature. The resulting mixture was stirred overnight at room temperature. To the above mixture was added NaBH(OAc)₃ (1131.07 mg, 5.336 mmol, 4 equiv) at room temperature. The resulting mixture was stirred for additional 6 h at room temperature. The reaction was quenched with 10% HOAc (aq.) at room temperature. The aqueous layer was extracted with EtOAc (50 mL). The aqueous layer was filtered, the filter cake was washed with water (10 mL). The filtrate was concentrated under reduced pressure. The residue was purified by reverse flash chromatography with the following conditions (column, C18 silica gel; mobile phase, MeCN in water (0.1% FA), 10% to 50% gradient in 20 min; detector, UV 254 nm) to afford the crude product. The crude product was purified by Prep-HPLC with the following conditions (Column: XSelect CSH Prep C18 OBD Column, 19*250 mm, 5 μm; Mobile Phase A: Water (0.1% FA) Water (0.1% FA), Mobile Phase B: ACN; Flow rate: 25 mL/min; Gradient: 2% B to 10% B in 15 min, Wave Length: 254; 220 nm; RT1 (min): 13.77) to afford Compound 113 (4.5 mg, 0.30%) as a yellow solid.

LC-MS-Compound 113: (ES, m/z): [M−2HCOOH+H]⁺ 966

H-NMR-Compound 113: (400 MHz, DMSO-d6, δ ppm): 1.51-1.72 (m, 2H), 1.78-2.02 (m, 4H), 2.29-2.42 (m, 3H), 2.53-2.63 (m, 3H), 2.85-2.99 (m, 4H), 3.16-3.32 (m, 7H), 3.45-3.57 (m, 7H), 3.85-4.05 (m, 5H), 4.05-4.35 (m, 6H), 4.91-5.08 (m, 5H), 6.92-6.94 (d, 1H), 7.06-7.13 (m, 3H), 7.33-7.54 (m, 4H), 7.72-7.77 (m, 2H), 8.21 (s, 1H), 8.35 (s, 2H), 10.95 (s, 1H).

### Example 112. Compound 114

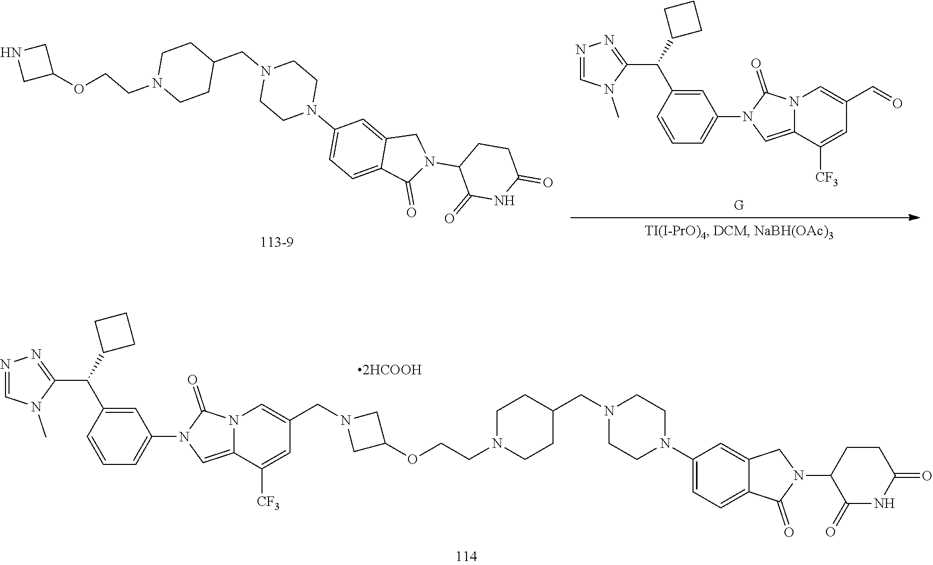 [SMILES: O=C1CCC(N2Cc3cc(N4CCN(CC5CCN(CCOC6CNC6)CC5)CC4)ccc3C2=O)C(=O)N1 ; Cn1cnnc1[C@@H](c1cccc(-n2cc3c(C(F)(F)F)cc(C=O)cn3c2=O)c1)C1CCC1 ; Cn1cnnc1[C@@H](c1cccc(-n2cc3c(C(F)(F)F)cc(CN4CC(OCCN5CCC(CN6CCN(c7ccc8c(c7)CN(C7CCC(=O)NC7=O)C8=O)CC6)CC5)C4)cn3c2=O)c1)C1CCC1 ; "·2HCOOH"]

### Synthesis of Compound 114.

To a stirred solution of Compound 113-9 (700 mg, 1.334 mmol, 1 equiv) and Intermediate G (607.64 mg, 1.334 mmol, 1 equiv) in DCM (20 mL) was added Ti(Oi-Pr)₄ (1137.60 mg, 4.002 mmol, 3 equiv) at room temperature. The resulting mixture was stirred overnight at room temperature. To the above mixture was added NaBH(OAc)₃ (1131.07 mg, 5.336 mmol, 4 equiv) at room temperature. The resulting mixture was stirred for additional 6 h at room temperature. The reaction was quenched with 10% HOAc (aq.) at room temperature. The aqueous layer was extracted with EtOAc (50 mL). The aqueous layer was filtered, the filter cake was washed with water (10 mL). The filtrate was concentrated under reduced pressure. The residue was purified by reverse flash chromatography with the following conditions (column, C18 silica gel; mobile phase, MeCN in water (0.1% FA), 10% to 50% gradient in 20 min; detector, UV 254 nm) to afford the crude product. The crude product was purified by Prep-HPLC with the following conditions (Column: XSelect CSH Prep C18 OBD Column, 19*250 mm, 5 μm; Mobile Phase A: Water (0.1% FA) Water (0.1% FA), Mobile Phase B: ACN; Flow rate: 25 mL/min; Gradient: 3% B to 17% B in 15 min; Wave Length: 254; 220 nm; RT1 (min): 12.58) to afford Compound 114 (7 mg, 0.49%) as a yellow solid.

LC-MS-Compound 114: (ES, m/z): [M−2HCOOH+H]⁺ 964

H-NMR-Compound 114: (400 MHz, DMSO-d6, δ ppm): 1.51-1.62 (m, 2H), 1.62-1.89 (m, 7H), 1.90-2.01 (m, 2H), 2.04-2.17 (m, 1H), 2.29-2.42 (m, 3H), 2.53-2.63 (m, 3H), 2.85-2.95 (m, 1H), 3.16-3.32 (m, 8H), 3.45-3.50 (m, 4H), 3.51-3.57 (m, 5H), 3.89-4.15 (m, 6H), 4.15-4.25 (m, 5H), 5.03-5.11 (d, 1H), 7.06-7.13 (m, 3H), 7.21-7.23 (d, 1H), 7.33 (s, 1H), 7.43-7.47 (m, 1H), 7.52-7.54 (d, 1H), 7.69-7.72 (m, 3H), 8.33-8.37 (m, 3H), 10.95 (s, 1H).

### Example 113. Compound 115

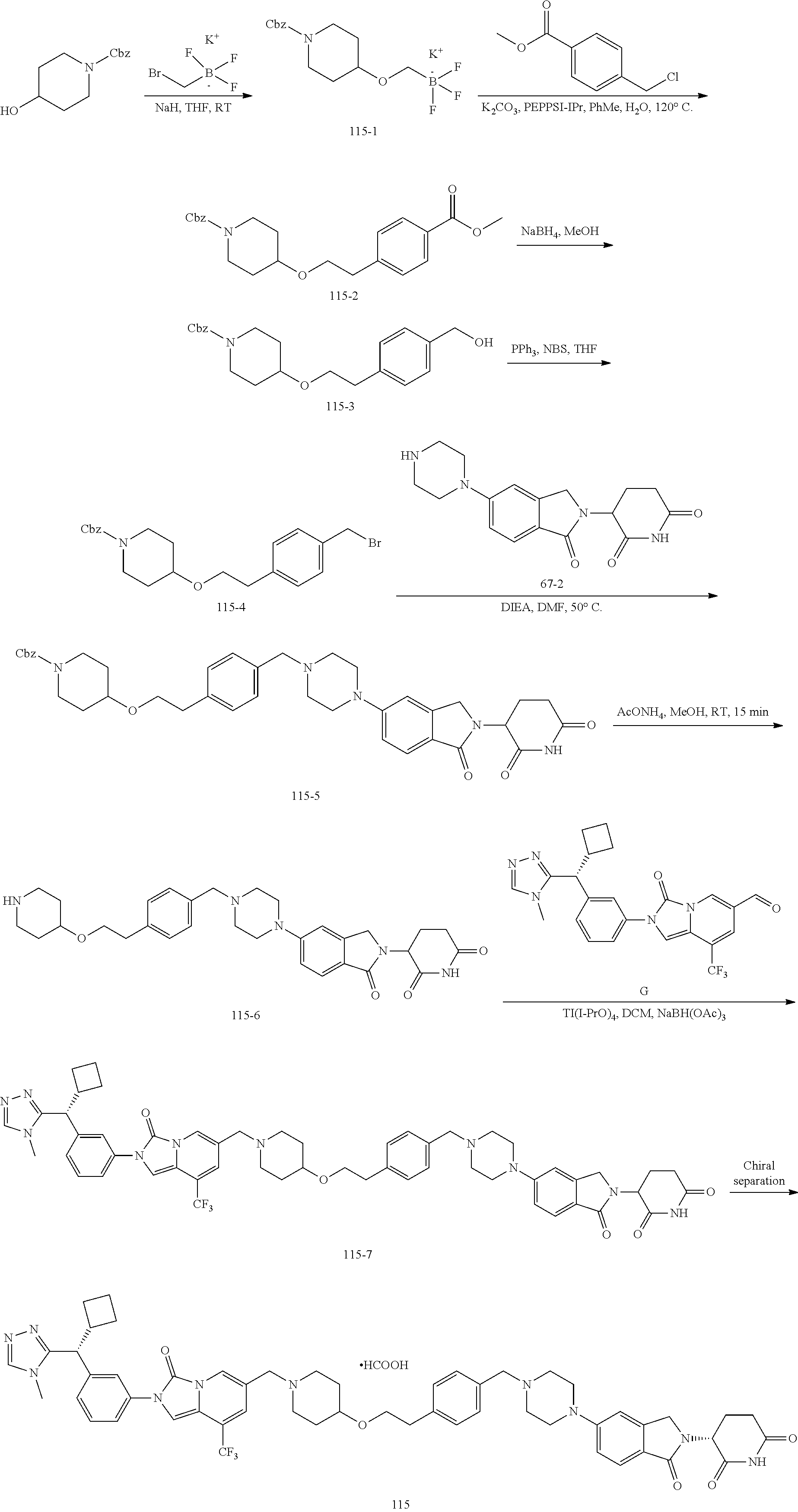

### Synthesis of Compound 115-1.

To a stirred mixture of NaH (13.6 g, 340.016 mmol, 2 equiv, 60%) in THF (400 mL) was added benzyl 4-hydroxypiperidine-1-carboxylate (40 g, 170.008 mmol, 1 equiv) at 0° C. The resulting mixture was stirred for 30 min at 0° C. To the above mixture was added potassium(bromomethyl)trifluoroboranuide (51.22 g, 255.012 mmol, 1.5 equiv) at 0° C. The resulting mixture was stirred overnight at room temperature. The resulting mixture was concentrated under vacuum. The residue was dissolved in acetone (500 mL). The resulting mixture was stirred for 3 h at 60° C. The mixture was allowed to cool down to room temperature. The resulting mixture was filtered, the filter cake was washed with acetone (2×25 ml). The filtrate was concentrated under reduced pressure. This resulted in Compound 115-1 (50 g, 82.80%) as a brown oil.

### Synthesis of Compound 115-2.

To a stirred mixture of Compound 115-1 (20 g, 56.305 mmol, 1 equiv) and methyl 4-(chloromethyl)benzoate (20.79 g, 112.610 mmol, 2 equiv) in Toluene (190 mL)/H₂O (10 mL) was added K₂CO₃ (23.34 g, 168.915 mmol, 3 equiv) and PEPPSI-IPr (4.46 g, 5.631 mmol, 0.1 equiv) at room temperature. The resulting mixture was stirred overnight at 120° C. The mixture was allowed to cool down to room temperature. The resulting mixture was diluted with water (200 mL). The aqueous layer was extracted with EtOAc (3×100 mL). The resulting mixture was concentrated under vacuum. The residue was purified by silica gel column chromatography, eluted with PE/EA (5:1) to afford Compound 115-2 (11 g, 49.15%) as a yellow oil.

### Synthesis of Compound 115-3.

To a stirred solution of Compound 15-2 (10 g, 25.159 mmol, 1 equiv) in MeOH (100 mL) was added NaBH₄ (0.95 g, 25.159 mmol, 1 equiv) in portions at 0° C. The resulting mixture was stirred overnight at room temperature. The reaction was quenched with NH₄Cl (200 mL) at room temperature. The aqueous layer was extracted with EtOAc (3×200 mL). The resulting mixture was concentrated under vacuum. The residue was purified by silica gel column chromatography, eluted with PE/EA (5:1) to afford Compound 115-3 (10 g, 95.75%) as a white solid.

### Synthesis of Compound 115-4.

To a stirred mixture of Compound 115-3 (10 g, 27.066 mmol, 1 equiv) and PPh₃ (10.65 g, 40.599 mmol, 1.5 equiv) in THF (100 mL) was added NBS (7.23 g, 40.599 mmol, 1.5 equiv) in portions at room temperature. The resulting mixture was stirred for 4 h at room temperature. The reaction was quenched with water (400 mL) at room temperature. The aqueous layer was extracted with EtOAc (3×200 mL). The resulting mixture was concentrated under vacuum. The residue was purified by silica gel column chromatography, eluted with PE/EA (10:1) to afford Compound 115-4 (7 g, 59.82%) as a yellow solid.

### Synthesis of Compound 115-5.

To a stirred mixture of Compound 115-4 (4 g, 9.252 mmol, 1 equiv) and Compound 67-2 (3.34 g, 10.177 mmol, 1.1 equiv) in DMF (40 mL) was added DIEA (2.39 g, 18.504 mmol, 2 equiv) at room temperature. The resulting mixture was stirred for 4 h at 50° C. The mixture was allowed to cool down to room temperature. The residue was purified by reversed-phase flash chromatography with the following conditions: column, C18 silica gel; mobile phase, MeCN in water (0.1% FA), 10% to 60% gradient in 35 min; detector, UV 254 nm. This resulted in Compound 115-5 (3 g, 47.70%) as a yellow solid.

### Synthesis of Compound 115-6.

To a stirred mixture of Compound 115-5 (1.5 g, 2.206 mmol, 1 equiv) and AcONH₄ (1.70 g, 22.060 mmol, 10 equiv) in MeOH (300 mL) was added Pd/C (0.15 g, 10%) at room temperature under nitrogen atmosphere. The resulting mixture was stirred for 15 min at room temperature under hydrogen atmosphere. The resulting mixture was filtered, the filter cake was washed with MeOH (3×30 mL). The filtrate was concentrated under reduced pressure. The residue was purified by reversed-phase flash chromatography with the following conditions: column, C18 silica gel; mobile phase, MeCN in water (0.1% FA), 10% to 40% gradient in 30 min; detector, UV 220 nm. This resulted in Compound 115-6 (1 g, 83.05%) as a white solid.

### Synthesis of Compound 115-7.

To a stirred mixture of Compound 115-6 (1 g, 1.833 mmol, 1 equiv) and Intermediate G (1.09 g, 2.383 mmol, 1.3 equiv) in DCM (20 mL) was added Ti(Oi-Pr)₄ (1.04 g, 3.666 mmol, 2 equiv) at room temperature. The resulting mixture was stirred overnight at room temperature. To the above mixture was added NaBH(OAc)₃ (0.78 g, 3.666 mmol, 2 equiv) at room temperature. The resulting mixture was stirred for additional 4 h at room temperature. The reaction was quenched with 10% HOAc(aq.) (150 ml) at room temperature. The resulting mixture was filtered, the filter cake was washed with 10% HOAc(aq.) (2×25 mL). The filtrate was concentrated under reduced pressure. The residue was purified by reversed-phase flash chromatography with the following conditions: column, C18 silica gel; mobile phase, MeCN in water (0.1% FA), 10% to 40% gradient in 20 min; detector, UV 254 nm. This resulted in Compound 115-7 (350 mg, 19.39%) as a yellow solid.

### Synthesis of Compound 115-8.

The Compound 115-7 (350 mg) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 70% B to 70% B in 10.5 min; Wave Length: 220/254 nm; RT1 (min): 5.42; RT2 (min): 8.90; the first peak is product) to afford the crude product. The crude product was purified by reversed-phase flash chromatography with the following conditions: column, C18 silica gel; mobile phase, MeCN in water (0.1% FA), 10% to 40% gradient in 20 min; detector, UV 254 nm. This resulted in Compound 115 (97.1, 27.74%) as a yellow solid.

LC-MS-Compound 115: (ES, m/z): [M−HCOOH+H]⁺ 985

H-NMR-Compound 115: (400 MHz, DMSO-d6, δ ppm): 1.41-1.43 (m, 2H), 1.79-1.80 (m, 7H), 1.91-1.97 (m, 1H), 2.09-2.11 (m, 3H), 2.33-2.42 (m, 2H), 2.51-2.60 (m, 2H), 2.76-2.79 (m, 2H), 2.91-2.93 (m, 1H), 3.25-3.28 (m, 10H), 3.42-3.49 (m, 6H), 3.58-3.61 (m, 3H), 4.16-4.33 (m, 3H), 5.03-5.05 (d, 1H), 7.00-7.05 (m, 3H), 7.20-7.32 (m, 6H), 7.42-7.51 (m, 2H), 7.64-7.73 (m, 3H), 8.23 (s, 1H), 8.32 (s, 1H), 10.95 (s, 1H).

### Example 114. Compound 116

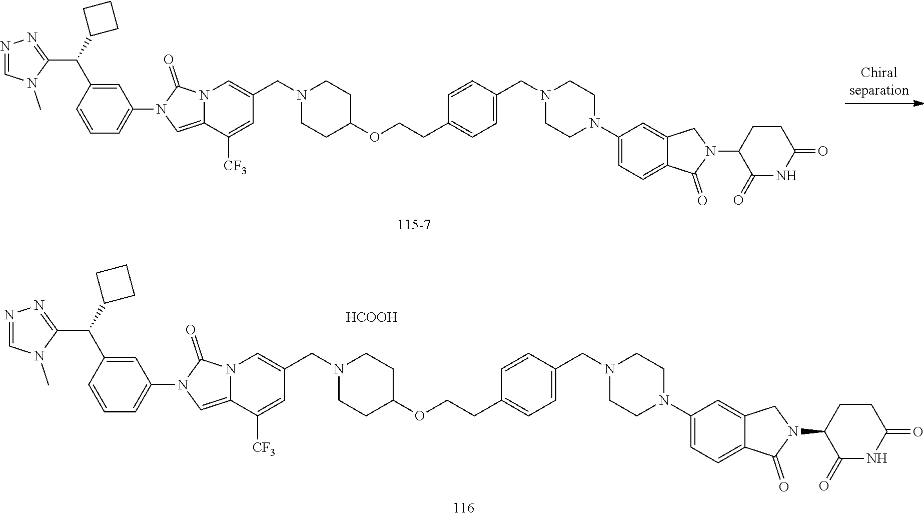 [SMILES: Cn1cnnc1[C@@H](c1cccc(-n2cc3c(C(F)(F)F)cc(CN4CCC(OCCc5ccc(CN6CCN(c7ccc8c(c7)CN(C7CCC(=O)NC7=O)C8=O)CC6)cc5)CC4)cn3c2=O)c1)C1CCC1 ; Cn1cnnc1[C@@H](c1cccc(-n2cc3c(C(F)(F)F)cc(CN4CCC(OCCc5ccc(CN6CCN(c7ccc8c(c7)CN([C@H]7CCC(=O)NC7=O)C8=O)CC6)cc5)CC4)cn3c2=O)c1)C1CCC1 ; "HCOOH"]

### Synthesis of Compound 116.

The Compound 115-7 (350 mg) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 70% B to 70% B in 10.5 min; Wave Length: 220/254 nm; RT1 (min): 5.42; RT2 (min): 8.90; the second peak is product) to afford the crude product. The crude product was purified by reversed-phase flash chromatography with the following conditions: column, C18 silica gel; mobile phase, MeCN in water (0.1% FA), 10% to 40% gradient in 20 min; detector, UV 254 nm. This resulted in Compound 116 (94.9 mg, 27.11%) as a yellow solid.

LC-MS-Compound 116: (ES, m/z): [M−HCOOH+H]⁺ 985

H-NMR-Compound 116: (400 MHz, DMSO-d6, δ ppm): 1.41-1.43 (m, 2H), 1.79-1.80 (m, 7H), 1.91-1.97 (m, 1H), 2.09-2.11 (m, 3H), 2.33-2.34 (m, 2H), 2.51-2.60 (m, 2H), 2.76-2.93 (m, 3H), 3.25-3.28 (m, 10H), 3.42-3.49 (m, 6H), 3.58-3.61 (m, 3H), 4.16-4.33 (m, 3H), 5.03-5.05 (d, 1H), 7.00-7.05 (m, 3H), 7.20-7.25 (m, 5H), 7.31 (s, 1H), 7.42-7.51 (m, 2H), 7.64-7.73 (m, 3H), 8.23 (s, 1H), 8.32 (s, 1H), 10.95 (s, 1H).

### Example 115. Compound 117

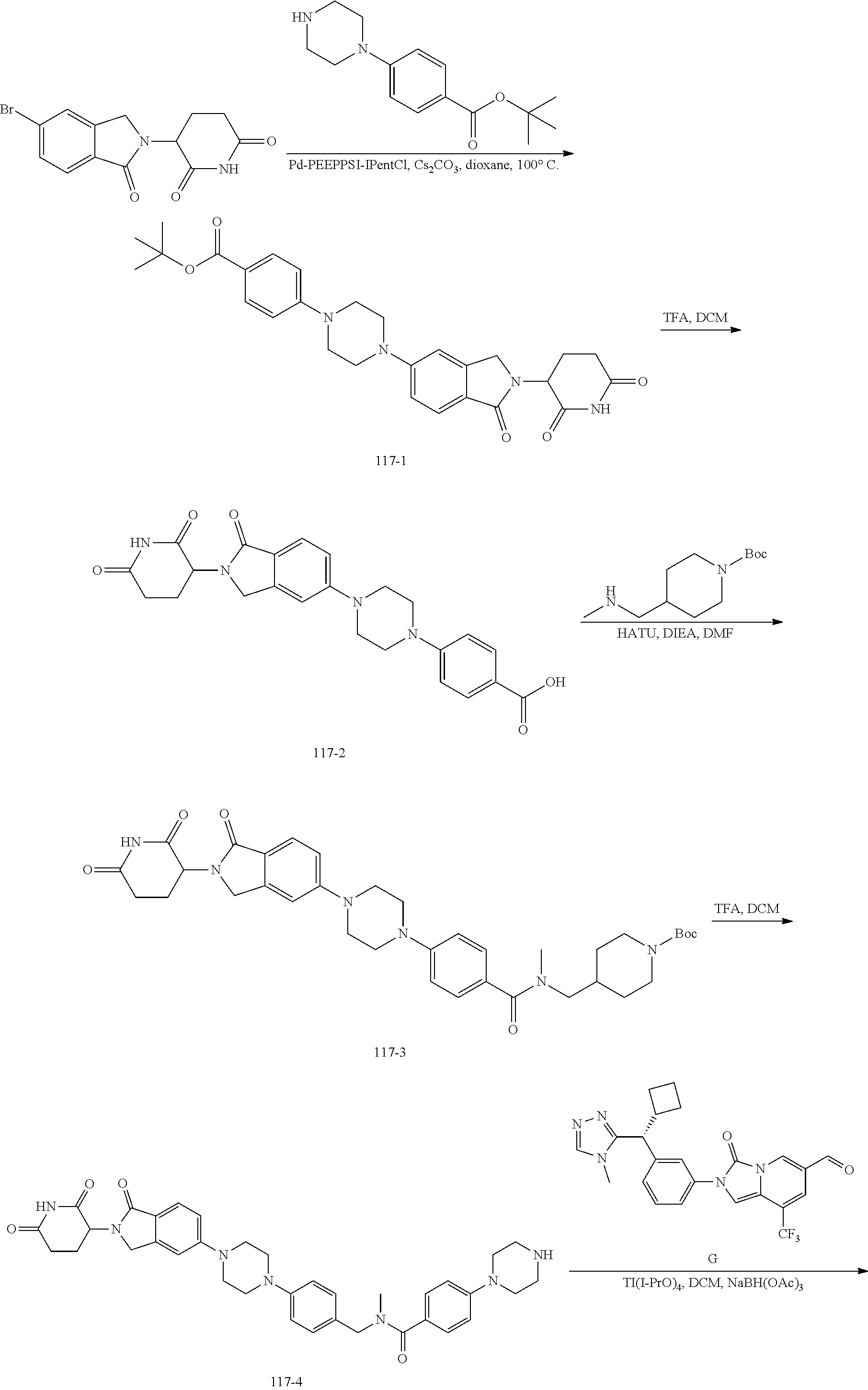

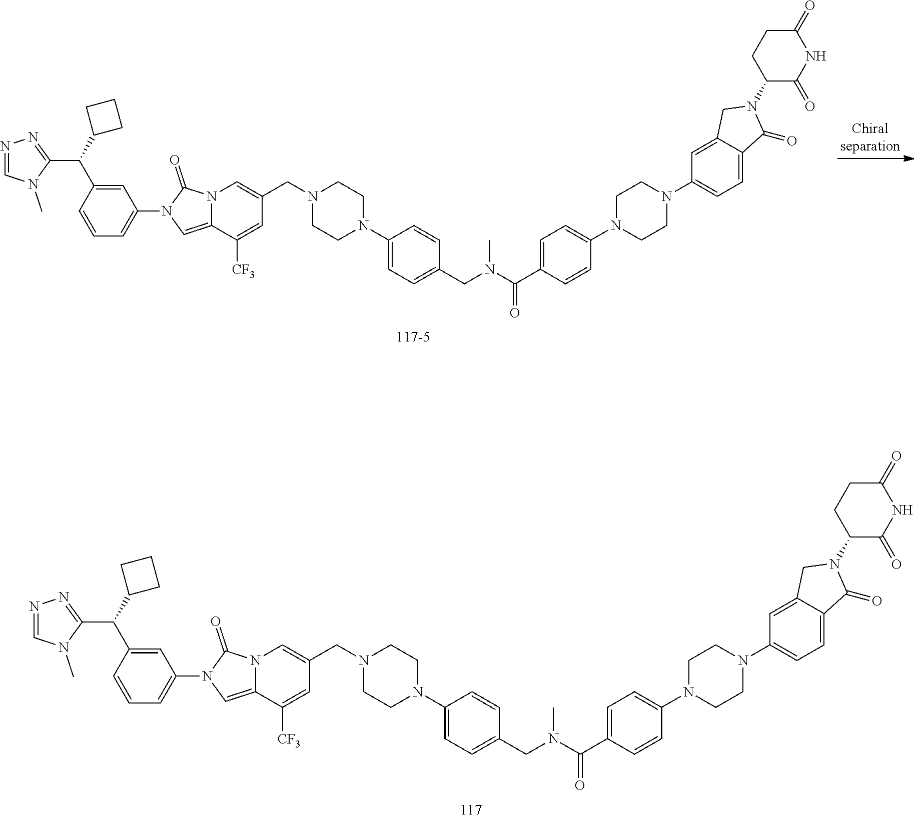 [SMILES: CN(Cc1ccc(N2CCN(Cc3cc(C(F)(F)F)c4cn(-c5cccc([C@H](c6nncn6C)C6CCC6)c5)c(=O)n4c3)CC2)cc1)C(=O)c1ccc(N2CCN(c3ccc4c(c3)CN([C@@H]3CCC(=O)NC3=O)C4=O)CC2)cc1 ; CN(Cc1ccc(N2CCN(Cc3cc(C(F)(F)F)c4cn(-c5cccc([C@H](c6nncn6C)C6CCC6)c5)c(=O)n4c3)CC2)cc1)C(=O)c1ccc(N2CCN(c3ccc4c(c3)CN([C@@H]3CCC(=O)NC3=O)C4=O)CC2)cc1]

### Synthesis of Compound 117-1.

Into a 500 mL 3-necked round-bottom flask were added 3-(5-bromo-1-oxo-3H-isoindol-2-yl) piperidine-2,6-dione (20 g, 61.892 mmol, 1 equiv), tert-butyl 4-(piperazin-1-yl) benzoate (19.48 g, 74.270 mmol, 1.2 equiv), Pd-PEPPSI-IPentCl (2.60 g, 3.095 mmol, 0.05 equiv), Cs₂CO₃ (40.33 g, 123.784 mmol, 2 equiv) and dioxane (300 mL) at room temperature. The resulting mixture was stirred for overnight at 100° C. under nitrogen atmosphere. The resulting mixture was diluted with AcOH/H₂O (1:10) (1000 mL). The aqueous layer was extracted with DCM (3×400 mL). The resulting mixture was concentrated under vacuum. The residue was purified by silica gel column chromatography, eluted with DCM/MeOH (80:1) to afford Compound 117-1 (22 g, 66.22%) as a brown solid.

### Synthesis of Compound 117-2.

Into a 250 mL round-bottom flask were added Compound 117-1 (9 g, 17.836 mmol, 1 equiv), TFA (20 mL) and DCM (80 mL) at room temperature. The resulting mixture was stirred for 3 h at room temperature. The resulting mixture was diluted with DCM (300 mL). The resulting mixture was concentrated under vacuum. The residue was purified by trituration with MeCN (50 mL). This resulted in Compound 117-2 (6.5 g, 76.38%) as a brown solid.

### Synthesis of Compound 117-3.

Into a 250 mL round-bottom flask were added Compound 117-2 (3.5 g, 7.804 mmol, 1 equiv), tert-butyl 4-[(methylamino)methyl] piperidine-1-carboxylate (2.14 g, 9.365 mmol, 1.2 equiv), HATU (5.93 g, 15.608 mmol, 2 equiv), DIEA (3.03 g, 23.412 mmol, 3 equiv) and DMF (50 mL) at room temperature. The resulting mixture was stirred for overnight at room temperature. The mixture/residue was acidified to pH 5 with AcOH (2 ml). The resulting mixture was concentrated under vacuum. The residue was purified by trituration with EtOAc (30 mL). This resulted in Compound 117-3 (3 g, 54.85%) as a brown solid.

### Synthesis of Compound 117-4.

Into a 100 mL round-bottom flask were added Compound 117-3 (3 g, 4.554 mmol, 1 equiv), TFA (8 mL) and DCM (25 mL) at room temperature. The resulting mixture was stirred for 2 h at room temperature. The resulting mixture was concentrated under vacuum. This resulted in Compound 117-4 (2.5 g, 91.39%) as a brown oil.

### Synthesis of Compound 117-5.

Into a 100 mL round-bottom flask were added Compound 117-4 (1.5 g, 2.685 mmol, 1 equiv), Intermediate G (1.83 g, 4.027 mmol, 1.5 equiv), Ti(Oi-Pr)₄ (3.82 g, 13.425 mmol, 5 equiv) and DCM (30 mL) at room temperature. The mixture was stirred for overnight at room temperature. To the above mixture was added STAB (1.71 g, 8.055 mmol, 3 equiv) at room temperature. The resulting mixture was stirred for 3 h at room temperature. The resulting mixture was diluted with 10% AcOH in water (200 mL). The aqueous layer was extracted with DCM (3×70 ml). The resulting mixture was concentrated under vacuum. The crude product was purified by Prep-HPLC with the following conditions (Column: X select CSH C18 OBD Column 30*150 mm 5 μm, n; Mobile Phase A: water (0.1% FA), Mobile Phase B: ACN; Flow rate: 60 mL/min; Gradient: 22% 13 to 33% B in 7 min, 33% B: Wave Length: 254; 220 nm; RT1 (min): 6.5) to afford Compound 117-5 (130 mg, 4.75%) as a yellow solid.

### Synthesis of Compound 117.

The Compound 117-5 (130 mg, 0.130 mmol, 1 equiv) was purified by Prep-Chiral-HPLC with the following conditions (Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 90% B to 90% B in 30 min; Wave Length: 220/254 nm; RT1 (min): 8.81; RT2 (min): 22.23. The first peak was the product. Sample Solvent: DCM:ACN=1:1—HPLC; Injection Volume: 1.55 mL; Number of Runs: 2) to afford Compound 117 (41.9 mg, 31.46%) as a yellow solid.

LC-MS-Compound 117: (ES, m/z): [M+H]⁺ 999

H-NMR-Compound 117: 1H NMR (400 MHz, DMSO-d6) δ1.67-1.79 (m, 8H), δ1.95-2.15 (m, 4H), δ2.32-2.39 (m, 1H), δ2.41-2.43 (m, 1H), δ2.75-2.97 (m, 6H), δ3.12-3.24 (m, 6H), δ3.42-3.59 (m, 12H), δ4.21-4.37 (m, 3H), δ5.04-5.08 (m, 1H), δ6.99-7.02 (m, 3H), δ7.12-7.18 (m, 3H), δ7.20-7.31 (d, 3H), δ7.42-7.45 (t, 1H), δ7.55-7.57 (d, 1H), δ7.66-7.73 (m, 3H), δ8.33 (s, 1H), 10.97 (s, 1H).

### Example 116. Compound 118

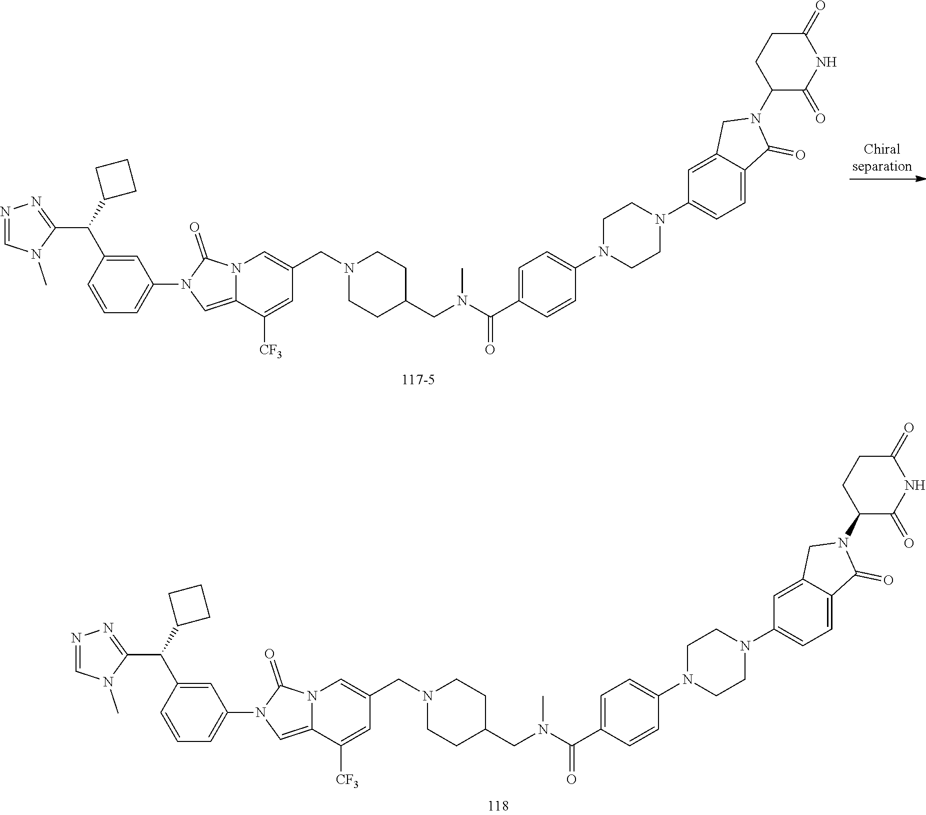 [SMILES: CN(CC1CCN(Cc2cc(C(F)(F)F)c3cn(-c4cccc([C@H](c5nncn5C)C5CCC5)c4)c(=O)n3c2)CC1)C(=O)c1ccc(N2CCN(c3ccc4c(c3)CN(C3CCC(=O)NC3=O)C4=O)CC2)cc1 ; CN(CC1CCN(Cc2cc(C(F)(F)F)c3cn(-c4cccc([C@H](c5nncn5C)C5CCC5)c4)c(=O)n3c2)CC1)C(=O)c1ccc(N2CCN(c3ccc4c(c3)CN([C@H]3CCC(=O)NC3=O)C4=O)CC2)cc1]

### Synthesis of Compound 118.

The Compound 117-5 (130 mg, 0.130 mmol, 1 equiv) was purified by Prep-Chiral-HPLC with the following conditions (Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 90% B to 90% B in 30 min; Wave Length: 220/254 nm; RT1 (min): 8.81; RT2 (min): 22.23. The second peak was the product. Sample Solvent: DCM:ACN=1:1—HPLC; Injection Volume: 1.55 mL; Number of Runs: 2) to afford Compound 118 (35.8 mg, 27.37%) as a yellow solid.

LC-MS-Compound 118: (ES, m/z): [M+H]⁺ 999

H-NMR-Compound 118: 1H NMR (400 MHz, DMSO-d6) δ1.67-1.79 (m, 8H), δ1.95-2.15 (m, 4H), δ2.32-2.39 (m, 1H), δ2.41-2.43 (m, 1H), δ2.75-2.97 (m, 6H), δ3.12-3.24 (m, 5H), δ3.42-3.59 (m, 13H), δ4.21-4.37 (m, 3H), δ5.04-5.08 (m, 1H), δ6.99-7.02 (m, 3H), δ7.12-7.18 (m, 3H), δ7.20-7.31 (d, 3H), δ7.42-7.45 (t, 1H), δ7.55-7.57 (d, 1H), δ7.66-7.73 (m, 3H), δ8.33 (s, 1H), 10.97 (s, 1H).

### Example 117. Compound 119

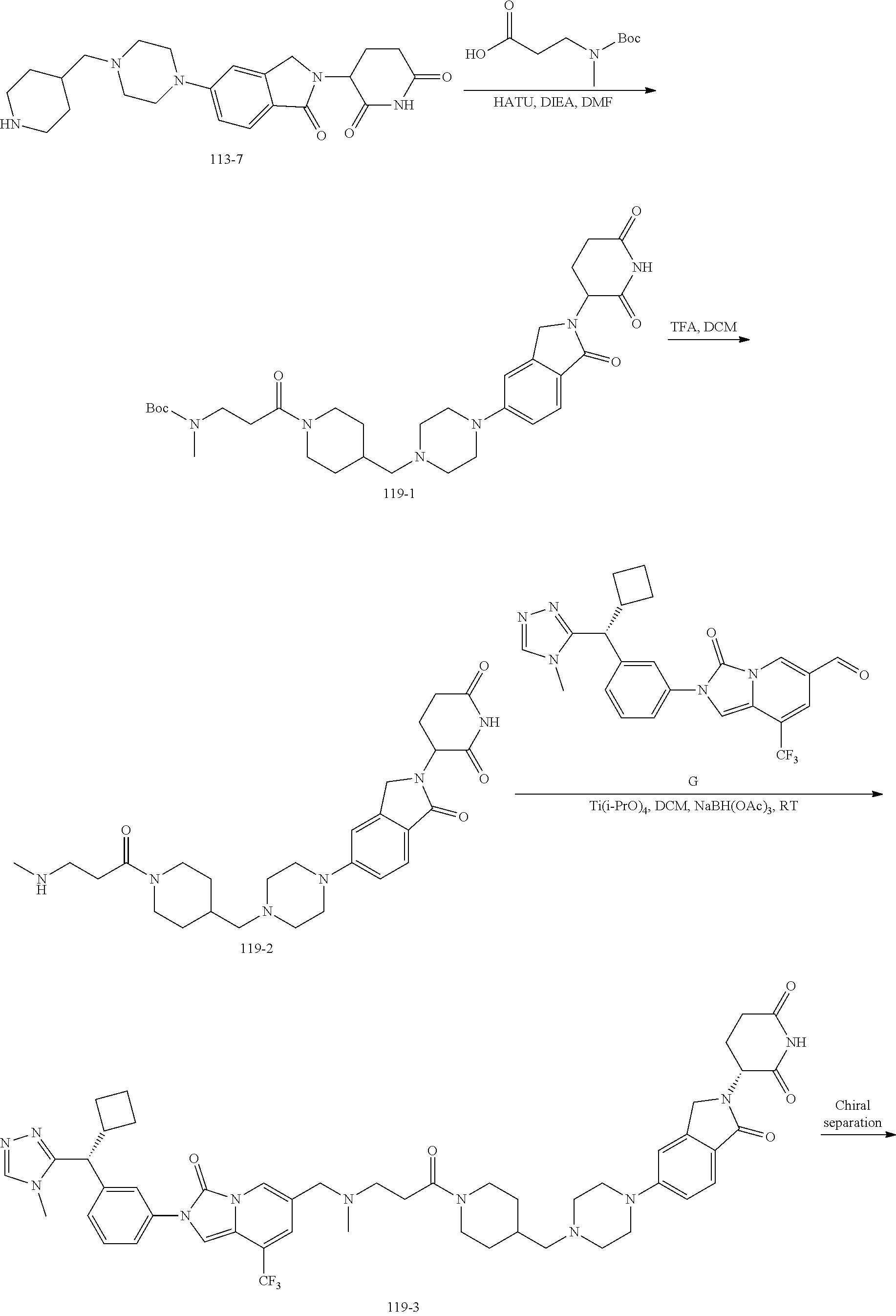

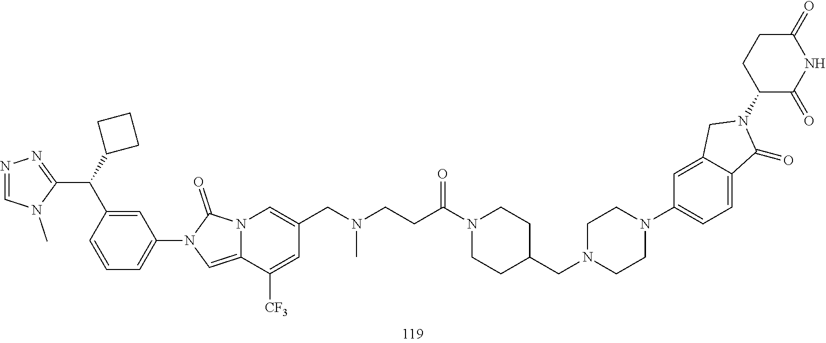 [SMILES: CN(CCC(=O)N1CCC(CN2CCN(c3ccc4c(c3)CN([C@@H]3CCC(=O)NC3=O)C4=O)CC2)CC1)Cc1cc(C(F)(F)F)c2cn(-c3cccc([C@H](c4nncn4C)C4CCC4)c3)c(=O)n2c1]

### Synthesis of Compound 119-1.

To a stirred solution of Compound 113-7 (1.5 g, 3.525 mmol, 1 equiv) and 3-[(tert-butoxycarbonyl)(methyl)amino]propanoic acid (0.86 g, 4.230 mmol, 1.2 equiv) in DMF (20 mL) was added HATU (1.74 g, 4.583 mmol, 1.3 equiv) and DIEA (0.91 g, 7.050 mmol, 2 equiv) at room temperature. The resulting mixture was stirred for 6 h at room temperature. The residue was purified by reverse flash chromatography with the following conditions: column, C18 silica gel; mobile phase, MeCN in water (0.1% FA), 0% to 60% gradient in 20 min; detector, UV 220 nm. This resulted in Compound 119-1 (1 g, 41.80%) as a brown solid.

### Synthesis of Compound 119-2.

A solution of Compound 119-1 (1 g, 1.637 mmol, 1 equiv) and TFA (5 mL) in DCM (15 mL) was stirred for 3 h at room temperature. The resulting mixture was concentrated under vacuum. The residue was purified by reverse flash chromatography with the following conditions: column, C18 silica gel; mobile phase, MeCN in water (0.1% FA), 0% to 50% gradient in 20 min; detector, UV 220 nm. This resulted in Compound 119-2 (800 mg, 88.99%) as a light brown solid.

### Synthesis of Compound 119-3.

To a stirred solution of Compound 119-2 (800 mg, 1.567 mmol, 1 equiv) and Intermediate G (713.52 mg, 1.567 mmol, 1 equiv) in DCM (25 mL) was added Ti(Oi-Pr)₄ (1335.83 mg, 4.701 mmol, 3 equiv) at room temperature. The resulting mixture was stirred overnight at room temperature. To the above mixture was added NaBH(OAc)₃ (1328.16 mg, 6.268 mmol, 4 equiv) at room temperature. The resulting mixture was stirred for additional 6 h at room temperature. The reaction was quenched with 10% HOAc (aq.) at room temperature. The aqueous layer was extracted with EtOAc (50 mL). The aqueous layer was filtered, the filter cake was washed with 10% HOAc (aq.) (10 mL). The filtrate was concentrated under reduced pressure. The residue was purified by reverse flash chromatography with the following conditions: column, C18 silica gel; mobile phase, MeCN in Water (0.1% FA), 10% to 50% gradient in 20 min; detector, UV 254 nm to afford the crude product. The crude product was purified by Prep-HPLC with the following conditions (Column: Xselect CSH C18 OBD Column 30*150 mm 5 μm; Mobile Phase A: Water (0.1% FA), Mobile Phase B: ACN; Flow rate: 60 mL/min; Gradient: 12% B to 23% B in 7 min, Wave Length: 254; 220 nm; RT1 (min): 6.03) to afford Compound 119-3 (280 mg, 18.25%) as a yellow solid.

### Synthesis of Compound 119.

The Compound 119-3 (280 mg) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 90% B to 90% B in 13 min; Wave Length: 220/254 nm; RT1 (min): 6.64; RT2 (min): 10.07; the first peak is product) to afford Compound 119 (92.5 mg, 31.55%) as a yellow solid.

LC-MS-Compound 119: (ES, m/z): [M+H]⁺ 950

H-NMR-Compound 119: (400 MHz, DMSO-d₆, δ ppm): 0.85-1.15 (m, 2H), 1.75-1.88 (m, 7H), 1.91-2.31 (m, 7H), 2.35-2.50 (m, 5H), 2.55-2.69 (m, 3H), 2.85-3.05 (m, 2H), 3.21-3.31 (m, 5H), 3.31-3.32 (m, 6H), 3.42 (s, 3H), 3.85-3.95 (m, 1H), 4.18-4.41 (m, 4H), 5.03-5.07 (m, 1H), 7.03-7.05 (m, 3H), 7.19-7.21 (d, 1H), 7.33 (s, 1H), 7.42-7.46 (m, 1H), 7.51-7.53 (d, 1H), 7.70-7.74 (m, 3H), 8.33 (s, 1H), 10.95 (s, 1H).

### Example 118. Compound 120

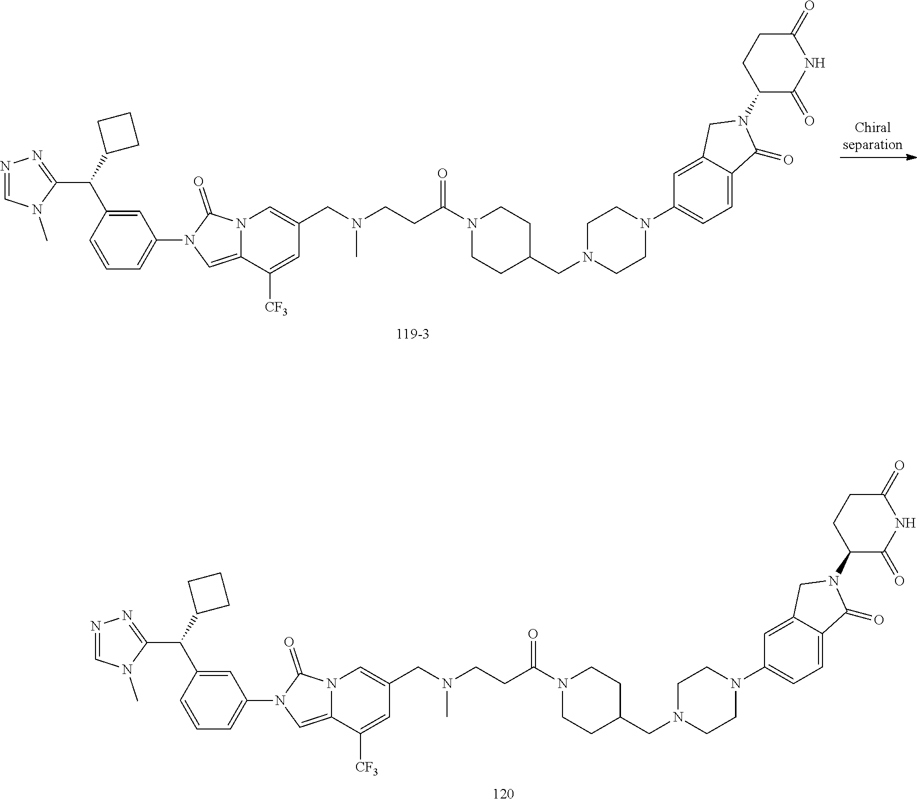 [SMILES: CN(CCC(=O)N1CCC(CN2CCN(c3ccc4c(c3)CN([C@@H]3CCC(=O)NC3=O)C4=O)CC2)CC1)Cc1cc(C(F)(F)F)c2cn(-c3cccc([C@H](c4nncn4C)C4CCC4)c3)c(=O)n2c1 ; CN(CCC(=O)N1CCC(CN2CCN(c3ccc4c(c3)CN([C@H]3CCC(=O)NC3=O)C4=O)CC2)CC1)Cc1cc(C(F)(F)F)c2cn(-c3cccc([C@H](c4nncn4C)C4CCC4)c3)c(=O)n2c1]

### Synthesis of Compound 120.

The Compound 119-3 (280 mg) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 90% B to 90% B in 13 min; Wave Length: 2201254 nm; RT1 (min): 6.64; RT2 (min): 10.07; the second peak is product) to afford Compound 120 (95.7 mg, 33.50%) as a yellow solid.

LC-MS-Compound 120: (ES, m/z): [M+H]⁺ 950

H-NMR-Compound 120: (400 MHz, DMSO-d₆, δ ppm): 0.85-1.15 (m, 2H), 1.75-1.88 (m, 7H), 1.91-2.08 (m, 1H), 2.08-2.19 (m, 3H), 2.20 (s, 3H), 2.35-2.45 (m, 1H), 2.45-2.50 (m, 4H), 2.55-2.69 (m, 3H), 2.85-3.05 (m, 2H), 3.21-3.31 (m, 5H), 3.31-3.32 (m, 6H), 3.42 (s, 3H), 3.85-3.95 (m, 1H), 4.18-4.41 (m, 4H), 5.03-5.07 (m, 1H), 7.03-7.05 (m, 3H), 7.19-7.21 (d, 1H), 7.33 (s, 1H), 7.42-7.46 (m, 1H), 7.51-7.53 (d, 1H), 7.70-7.74 (m, 3H), 8.33 (s, 1H), 10.95 (s, 1H).

### Example 119. Compound 121

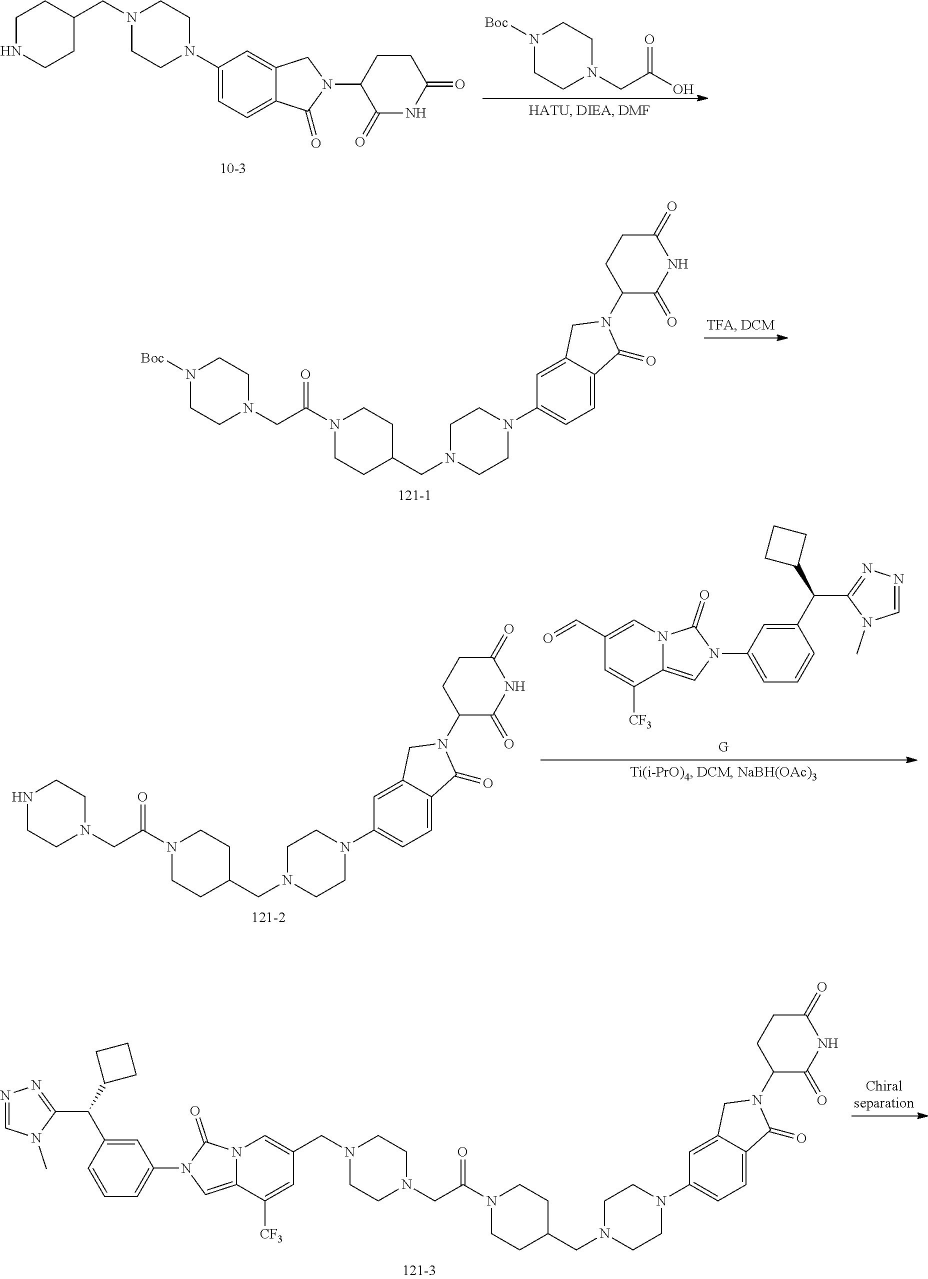

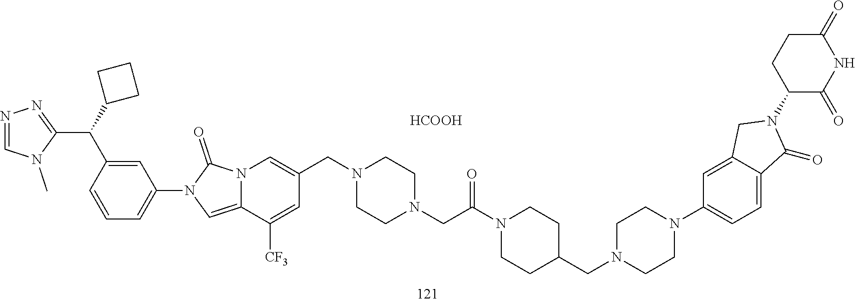 [SMILES: Cn1cnnc1[C@@H](c1cccc(-n2cc3c(C(F)(F)F)cc(CN4CCN(CC(=O)N5CCC(CN6CCN(c7ccc8c(c7)CN([C@@H]7CCC(=O)NC7=O)C8=O)CC6)CC5)CC4)cn3c2=O)c1)C1CCC1 ; "HCOOH"]

### Synthesis of Compound 121-1.

Into a 100 mL 3-necked round bottom flask were added Compound 10-3 (2.3 g, 9.40 mmol, 1.0 equiv), DMF (40 mL), HATU (5.3 g, 14.10 mmol, 1.5 equiv) and DIEA (3.6 g, 28.20 mmol, 3.0 equiv) at room temperature. The resulting mixture was stirred for 1 h at room temperature under air atmosphere. Into the mixture were added 3-{1-oxo-5-[4-(piperidin-4-ylmethyl) piperazin-1-yl]-3H-isoindol-2-yl} piperidine-2,6-dione (4 g, 9.40 mmol, 1.0 equiv) at room temperature. The resulting mixture was stirred for overnight at room temperature under air atmosphere. The reaction was quenched by the addition of water (100 mL) at room temperature. The aqueous layer was extracted with DCM/MeOH=10:1 (3×100 mL). The resulting mixture was concentrated under reduced pressure. The residue was purified by silica gel column chromatography, eluted with DCM/MeOH (20:1) to afford Compound 121-1 (1.6 g, 26%) as a yellow solid.

### Synthesis of Compound 121-2.

Into a 50 mL round bottom flask were added Compound 121-1 (1.6 g, 2.45 mmol, 1.0 equiv), TFA (4 mL) and DCM (12 mL) at room temperature. The resulting mixture was stirred for overnight at room temperature under N₂ atmosphere. The reaction was quenched by the addition of water (20 mL) at room temperature. The aqueous layer was extracted with DCM (2×5 mL). The combined water layers. The resulting mixture was concentrated under reduced pressure. This resulted in Compound 121-2 (800 mg, 59%) as a yellow solid.

### Synthesis of Compound 121-3.

To a stirred solution of Compound 121-2 (480 mg, 0.87 mmol, 1.0 equiv) and Intermediate G (475 mg, 1.04 mmol, 1.2 equiv) in DCM (5 mL) was added Ti(Oi-Pr)₄ (989 mg, 3.48 mmol, 4.0 equiv) at room temperature. The resulting mixture was stirred for overnight at room temperature. To the above mixture was added NaBH(OAc)₃ (553 mg, 2.61 mmol, 3.0 equiv) at room temperature. The resulting mixture was stirred for additional 2 h at room temperature. The reaction was quenched by the addition of 10% AcOH (100 mL) at room temperature. The aqueous layer was extracted with EtOAc (3×60 mL). The aqueous phase was concentrated under reduced pressure. The residue was purified by reversed-phase flash chromatography with the following conditions: column, C18 silica gel; mobile phase, MeCN in water (0.1% FA), 10% to 50% gradient in 30 min; detector, UV 254 nm. The crude product (500 mg) was purified by Prep-HPLC with the following conditions (Column: Sunfire prep C18 column, 30*150 mm, 5 μm; Mobile Phase A: Water (0.1% FA), Mobile Phase B: ACN; Flow rate: 60 mL/min; Gradient: 12% B to 24% B in 7 min, 24% B; Wave Length: 254; 220 nm; RT1 (min): 6.3; Number of Runs: 0) to afford Compound 121-3 (150 mg, 17%) as a yellow solid.

### Synthesis of Compound 121.

The Compound 121-3 (150 mg) was purified by Prep-CHIRAL-HPLC with the following conditions Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 90% B to 90% B in 20 min; Wave Length: 220/254 nm; RT1 (min): 9.52; RT2 (min): 15.15; Sample Solvent: DCM:ACN=1:1—HPLC; Injection Volume: 1 mL; Number Of Runs: 4. The first peak was the product. The residue was purified by reversed-phase flash chromatography with the following conditions: column, C18 silica gel; mobile phase, MeCN in water (0.1% FA), 10% to 50% gradient in 30 min; detector, UV 254 nm. This resulted in Compound 121 (51 mg, 33%) as a yellow solid.

LC-MS-Compound 121: (ES, m/z): [M-COOH]⁺ 991

H-NMR-Compound 121: (400 MHz, DMSO-d6, δ ppm): 0.85.95 (m, 1H), 1.02-1.10 (m, 1H), 1.76-1.80 (m, 8H), 1.90-1.92 (m, 1H), 2.05-2.08 (m, 1H), 2.16-2.18 (m, 2H), 2.33-2.39 (m, 7H), 2.50-2.60 (m, 4H), 2.90-3.01 (m, 3H), 3.21-3.28 (m, 12H), 3.39-3.42 (m, 3H), 3.99-4.03 (m, 1H), 4.18-4.24 (m, 2H), 4.27-4.35 (m, 2H), 5.03-5.07 (m, 1H), 7.00-7.06 (m, 3H), 7.18-7.20 (m, 1H), 7.31-7.73 (m, 6H), 8.19 (s, 1H), 8.32 (s, 1H), 10.94 (s, 1H).

### Example 120. Compound 122

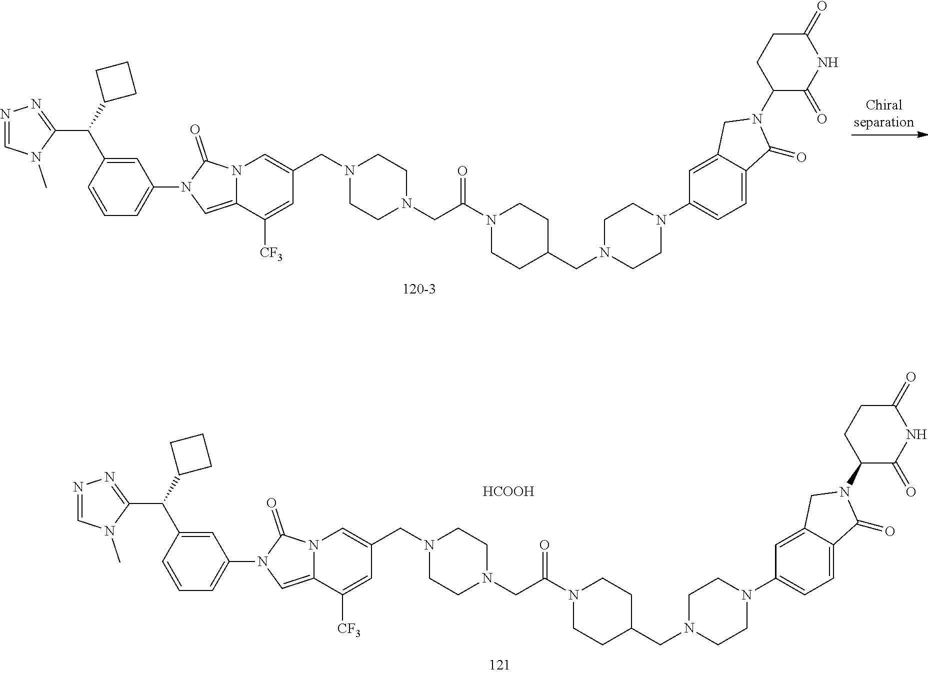 [SMILES: Cn1cnnc1[C@@H](c1cccc(-n2cc3c(C(F)(F)F)cc(CN4CCN(CC(=O)N5CCC(CN6CCN(c7ccc8c(c7)CN(C7CCC(=O)NC7=O)C8=O)CC6)CC5)CC4)cn3c2=O)c1)C1CCC1 ; Cn1cnnc1[C@@H](c1cccc(-n2cc3c(C(F)(F)F)cc(CN4CCN(CC(=O)N5CCC(CN6CCN(c7ccc8c(c7)CN([C@H]7CCC(=O)NC7=O)C8=O)CC6)CC5)CC4)cn3c2=O)c1)C1CCC1 ; "HCOOH"]

### Synthesis of Compound 121.

The Compound 120-3 (150 mg) was purified by Prep-CHIRAL-HPLC with the following conditions Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 90% B to 90% B in 20 min; Wave Length: 220/254 nm; RT1 (min): 9.52; RT2 (min): 15.15; Sample Solvent: DCM:ACN=1:1—HPLC; Injection Volume: 1 mL; Number Of Runs: 4. The second peak was the product. The residue was purified by reversed-phase flash chromatography with the following conditions: column, C18 silica gel; mobile phase, MeCN in water (0.1% FA), 10% to 50% gradient in 30 min; detector, UV 254 nm. This resulted in Compound 122 (46 mg, 33%) as a yellow solid.

LC-MS-Compound 122: (ES, m/z): [M-COOH]⁺ 991

H-NMR-Compound 122: (400 MHz, DMSO-d6, δ ppm): 0.85.95 (m, 1H), 1.02-1.10 (m, 1H), 1.76-1.80 (m, 8H), 1.90-1.92 (m, 1H), 2.05-2.08 (m, 1H), 2.16-2.18 (m, 2H), 2.33-2.39 (m, 7H), 2.50-2.60 (m, 4H), 2.90-3.01 (m, 3H), 3.21-3.28 (m, 12H), 3.39-3.42 (m, 3H), 3.99-4.03 (m, 1H), 4.18-4.24 (m, 2H), 4.27-4.35 (m, 2H), 5.03-5.07 (m, 1H), 7.00-7.06 (m, 3H), 7.18-7.20 (m, 1H), 7.31-7.73 (m, 6H), 8.19 (s, 1H), 8.32 (s, 1H), 10.94 (s, 1H).

### Example 121. Compound 123

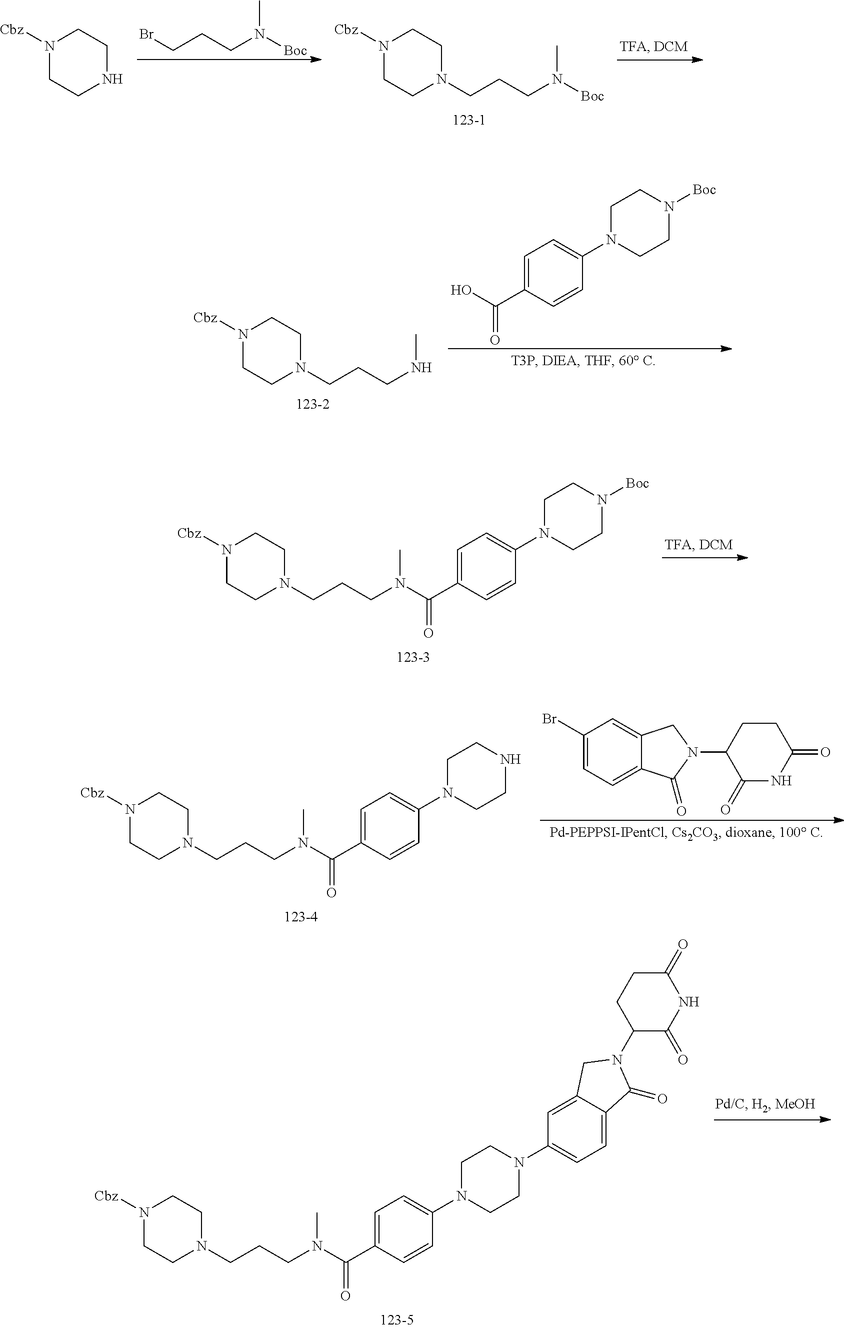

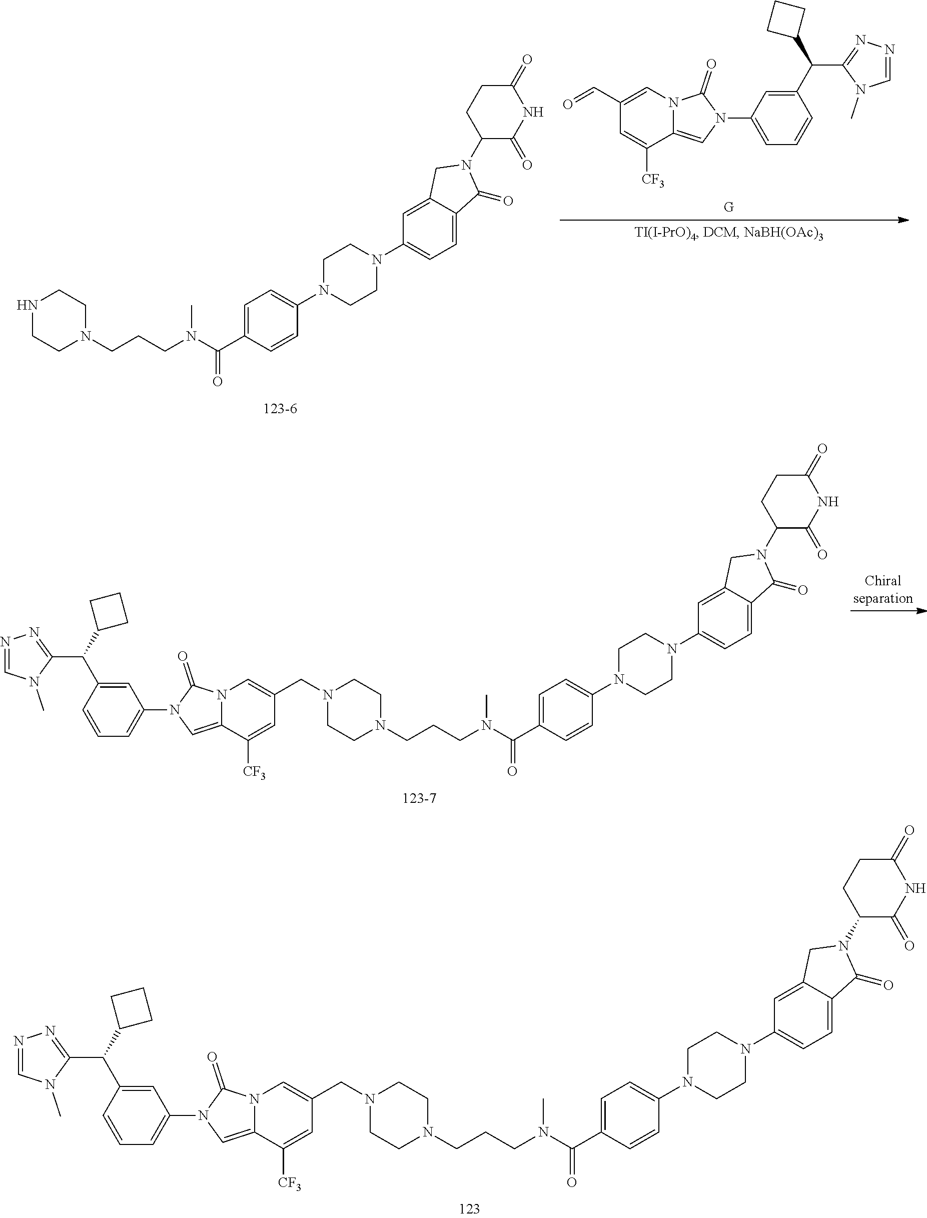

### Synthesis of Compound 123-1.

To a stirred solution of benzyl piperazine-1-carboxylate (5 g, 22.69 mmol, 1.0 equiv) and tert-butyl N-(3-bromopropyl)-N-methylcarbamate (6.8 g, 27.23 mmol, 1.2 equiv) in MeCN (50 mL) was added TEA (4.6 g, 45.39 mmol, 2.0 equiv) at room temperature. The resulting mixture was stirred for overnight at room temperature. The reaction was quenched by the addition of sat. NH₄Cl (aq.) (200 mL) at room temperature. The resulting mixture was extracted with EtOAc (3×200 mL). The combined organic layers were washed with water (3×200 mL), dried over anhydrous Na₂SO₄. After filtration, the filtrate was concentrated under reduced pressure. The residue was purified by silica gel column chromatography, eluted with PE/EA (5:1) to afford Compound 123-1 (3.2 g, 36%) as an off-white solid.

### Synthesis of Compound 123-2.

A solution of Compound 123-1 (3.2 g, 8.17 mmol, 1.0 equiv) and TFA (10 mL) in DCM (30 mL) was stirred for overnight at room temperature. The resulting mixture was concentrated under reduced pressure. The residue was purified by reversed-phase flash chromatography with the following conditions: column, C18 silica gel; mobile phase, MeCN in water (0.1% FA), 0% to 50% gradient in 30 min; detector, UV 254 nm. This resulted in Compound 123-2 (2.5 g, 94%) as an off-white solid.

### Synthesis of Compound 123-3.

To a stirred solution of Compound 123-2 (2.8 g, 9.60 mmol, 1.0 equiv) and 4-[4-(tert-butoxycarbonyl) piperazin-1-yl] benzoic acid (3.5 g, 11.53 mmol, 1.2 equiv) in THF (28 mL) were added T3P (4.6 g, 14.41 mmol, 1.5 equiv) and DIEA (3.7 g, 28.82 mmol, 3.0 equiv) at room temperature. The resulting mixture was stirred for 3 h at 60° C. The reaction was quenched by the addition of sat. NH₄Cl (aq.) (100 mL) at room temperature. The resulting mixture was extracted with EtOAc (3×100 mL). The combined organic layers were washed with water (3×100 mL), dried over anhydrous Na₂SO₄. After filtration, the filtrate was concentrated under reduced pressure. The residue was purified by silica gel column chromatography, eluted with PE/EA (1:1) to afford Compound 123-3 (2 g, 36%) as an off-white solid.

### Synthesis of Compound 123-4.

A solution of Compound 123-3 (1.2 g, 2.07 mmol, 1.0 equiv) and TFA (4 mL) in DCM (12 mL) was stirred for overnight at room temperature. The mixture was basified to pH 7 with NH₃ in MeOH. The resulting mixture was concentrated under reduced pressure. The residue was purified by Prep-TLC (CH₂Cl₂/MeOH=20:1) to afford Compound 123-4 (650 mg, 65%) as an off-white solid.

### Synthesis of Compound 123-5.

To a stirred solution of Compound 123-4 (1.2 g, 2.50 mmol, 1.0 equiv) and 3-(5-bromo-1-oxo-3H-isoindol-2-yl) piperidine-2,6-dione (1.6 g, 5.00 mmol, 2.0 equiv) in 1,4-dioxane (12 mL) were added Cs₂CO₃ (2.5 g, 7.50 mmol, 3.0 equiv) and Pd-PEPPSI-IPentCl 2-methylpyridine (o-picoline) (0.2 g, 0.25 mmol, 0.1 equiv) at room temperature under nitrogen atmosphere. The resulting mixture was stirred for overnight at 100° C. under nitrogen atmosphere. The reaction was quenched by the addition of 10% AcOH (100 mL) at room temperature. The aqueous layer was extracted with MeOH:CH₂Cl₂ (1:10) (3×100 mL). The resulting mixture was concentrated under reduced pressure. The residue was purified by Prep-TLC (CH₂Cl₂/MeOH=20:1) to afford Compound 123-5 (650 mg, 36%) as an off-white solid.

### Synthesis of Compound 123-6.

To a solution of Compound 123-6 (780 mg, 1.08 mmol, 1.0 equiv) in 24 mL MeOH was added Pd/C (10%, 230 mg) under nitrogen atmosphere in a 50 mL round-bottom flask. The mixture was hydrogenated at room temperature for 2 h under hydrogen atmosphere using a hydrogen balloon, filtered through a Celite pad and concentrated under reduced pressure. This resulted in Compound 123-6 (430 mg, 68%) as an off-white solid.

### Synthesis of Compound 123-7.

To a stirred solution of Compound 123-6 (410 mg, 0.69 mmol, 1.0 equiv) and Intermediate G (476 mg, 1.04 mmol, 1.5 equiv) in DCM (4 mL) was added Ti(Oi-Pr)₄ (793 mg, 2.79 mmol, 4.0 equiv) at room temperature. The resulting mixture was stirred for overnight at room temperature. To the above mixture was added NaBH(OAc)₃ (443 mg, 2.09 mmol, 3.0 equiv) at room temperature. The resulting mixture was stirred for additional 2 h at room temperature. The reaction was quenched by the addition of 10% AcOH (60 mL) at room temperature. The aqueous layer was extracted with EtOAc (2×60 mL). The aqueous phase was concentrated under reduced pressure. The residue was purified by reversed-phase flash chromatography with the following conditions: column, C18 silica gel; mobile phase, MeCN in water (0.1% FA), 10% to 50% gradient in 30 min; detector, UV 254 nm. This resulted in Compound 123-7 (130 mg, 18%) as a yellow solid.

### Synthesis of Compound 123.

The Compound 123-7 (120 mg) was purified by Prep-CHIRAL-HPLC with the following conditions Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 90% B to 90% B in 30 min; Wave Length: 220/254 nm; RT1 (min): 8.23; RT2 (min): 17.17; Sample Solvent: DCM:ACN=1:1—HPLC; Injection Volume: 1.5 mL; Number Of Runs: 2. The first peak was the product. This resulted in Compound 123 (30 mg, 25%) as a yellow solid.

LC-MS-Compound 123: (ES, m/z): [M+H]⁺ 1027

H-NMR-Compound 123: (400 MHz, DMSO-d6, δ ppm): 1.60-1.95 (m, 7H), 1.98-2.55 (m, 13H), 2.60-2.75 (m, 2H), 2.86-2.93 (m, 4H), 3.23-3.38 (m, 3H), 3.42-3.45 (m, 12H), 4.19-4.35 (m, 3H), 5.03-5.07 (m, 1H), 6.98-7.02 (m, 3H), 7.08-7.19 (m, 3H), 7.28-7.73 (m, 3H), 7.41-7.44 (m, 1H), 7.52-7.54 (m, 1H), 7.63-7.72 (m, 3H), 8.32 (s, 1H), 10.95 (s, 1H).

### Example 122. Compound 124

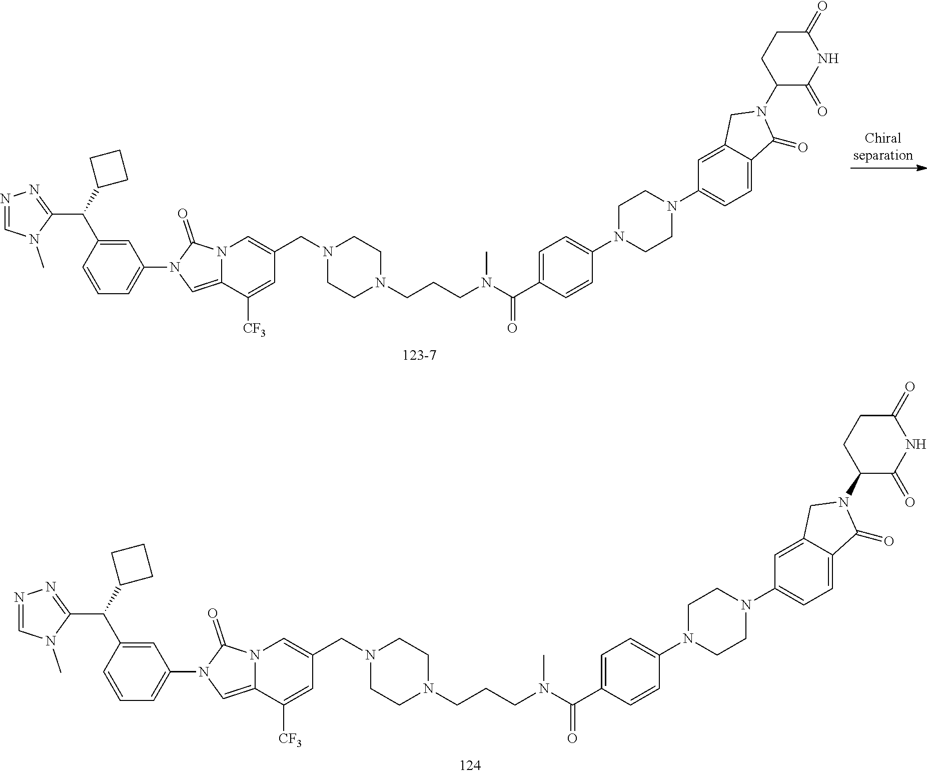 [SMILES: CN(CCCN1CCN(Cc2cc(C(F)(F)F)c3cn(-c4cccc([C@H](c5nncn5C)C5CCC5)c4)c(=O)n3c2)CC1)C(=O)c1ccc(N2CCN(c3ccc4c(c3)CN(C3CCC(=O)NC3=O)C4=O)CC2)cc1 ; CN(CCCN1CCN(Cc2cc(C(F)(F)F)c3cn(-c4cccc([C@H](c5nncn5C)C5CCC5)c4)c(=O)n3c2)CC1)C(=O)c1ccc(N2CCN(c3ccc4c(c3)CN([C@H]3CCC(=O)NC3=O)C4=O)CC2)cc1]

### Synthesis of Compound 124.

The Compound 123-7 (120 mg) was purified by Prep-CHIRAL-HPLC with the following conditions Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 90% B to 90% B in 30 min; Wave Length: 220/254 nm; RT1 (min): 8.23; RT2 (min): 17.17; Sample Solvent: DCM:ACN=1:1—HPLC; Injection Volume: 1.5 mL; Number Of Runs: 2. The second peak was the product. This resulted in Compound 124 (32 mg, 27%) as a yellow solid.

LC-MS-Compound 124: (ES, m/z): [M+H]⁺ 1027

H-NMR-Compound 124: (400 MHz, DMSO-d6, δ ppm): 1.60-1.95 (m, 7H), 1.98-2.55 (m, 13H), 2.60-2.75 (m, 2H), 2.86-2.93 (m, 4H), 3.23-3.38 (m, 3H), 3.42-3.45 (m, 12H), 4.19-4.35 (m, 3H), 5.03-5.07 (m, 1H), 6.98-7.02 (m, 3H), 7.08-7.19 (m, 3H), 7.28-7.73 (m, 3H), 7.41-7.44 (m, 1H), 7.52-7.54 (m, 1H), 7.63-7.72 (m, 3H), 8.32 (s, 1H), 10.95 (s, 1H).

### Example 123. Compound 125

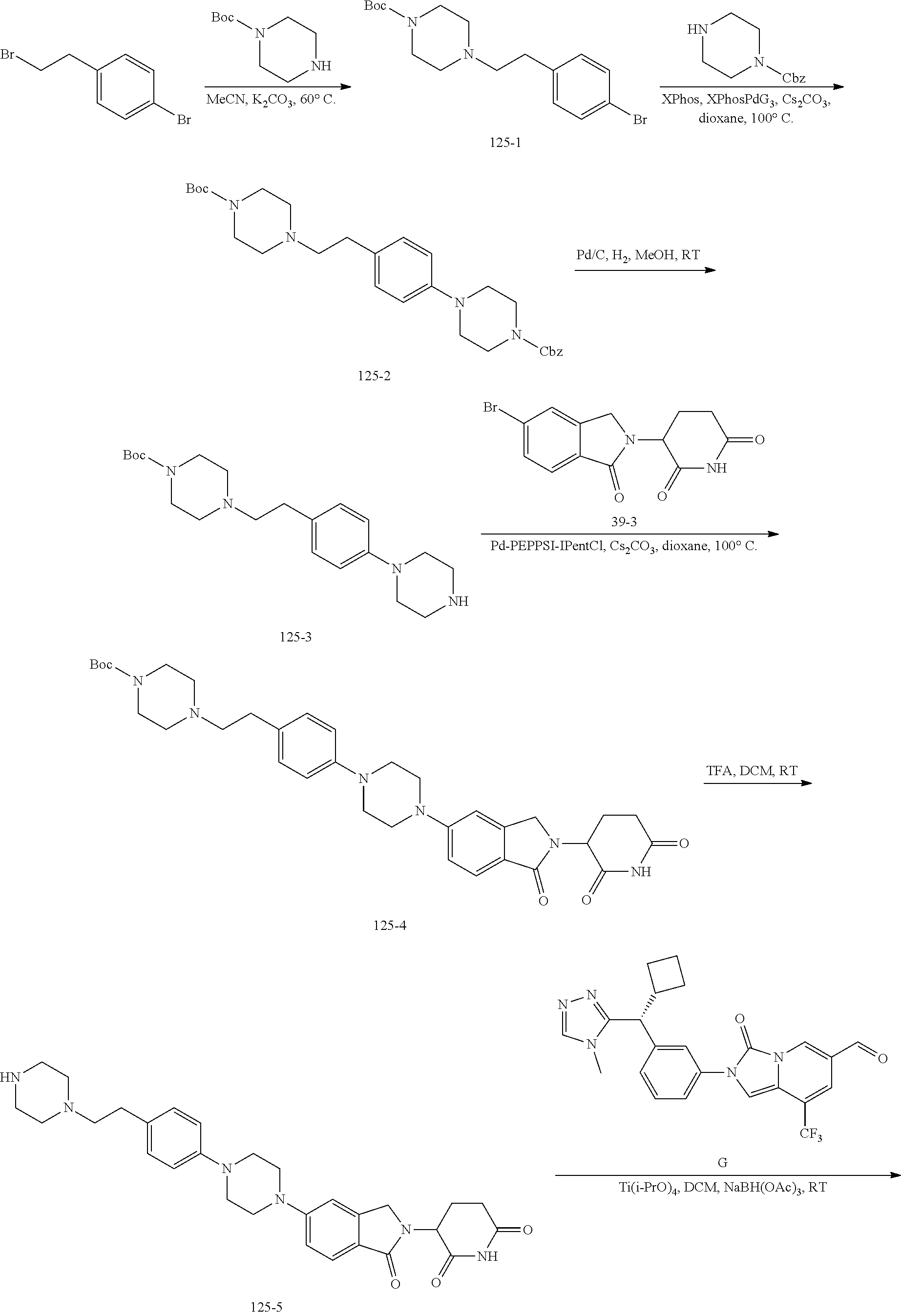

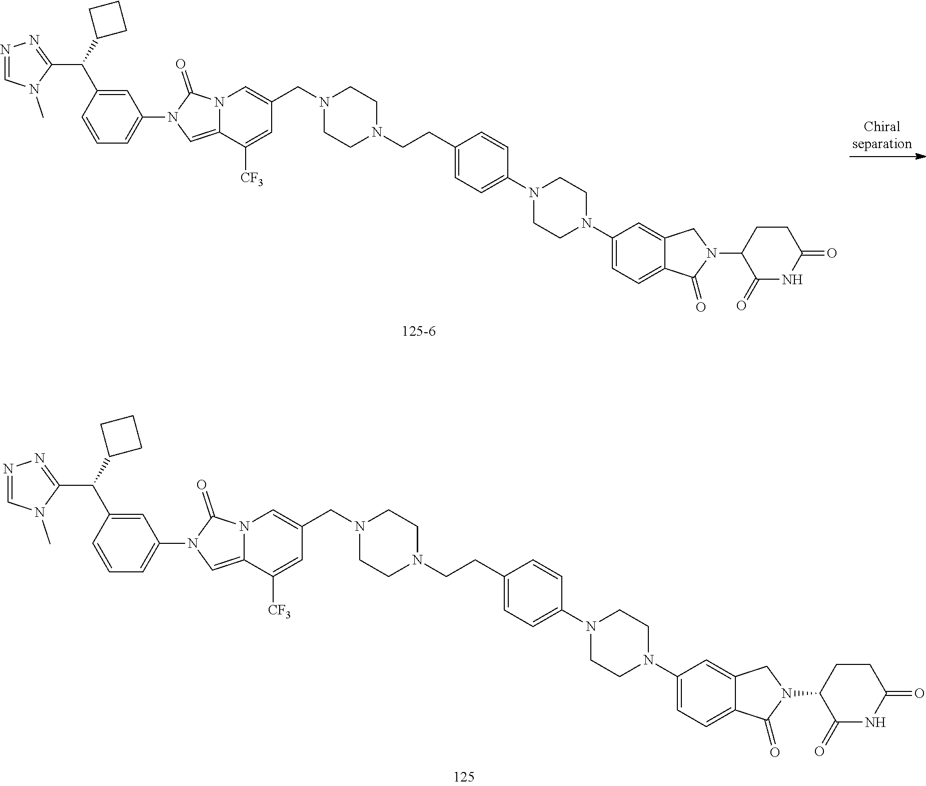 [SMILES: Cn1cnnc1[C@@H](c1cccc(-n2cc3c(C(F)(F)F)cc(CN4CCN(CCc5ccc(N6CCN(c7ccc8c(c7)CN(C7CCC(=O)NC7=O)C8=O)CC6)cc5)CC4)cn3c2=O)c1)C1CCC1 ; Cn1cnnc1[C@@H](c1cccc(-n2cc3c(C(F)(F)F)cc(CN4CCN(CCc5ccc(N6CCN(c7ccc8c(c7)CN([C@@H]7CCC(=O)NC7=O)C8=O)CC6)cc5)CC4)cn3c2=O)c1)C1CCC1]

### Synthesis of Compound 125-1.

To a stirred solution of 1-bromo-4-(2-bromoethyl) benzene (15 g, 56.827 mmol, 1 equiv) and tert-butyl piperazine-1-carboxylate (15.88 g, 85.240 mmol, 1.5 equiv) in MeCN (150 mL) was added K₂CO₃ (19.63 g, 142.067 mmol, 2.5 equiv) at room temperature. The resulting mixture was stirred for overnight at 60° C. under nitrogen atmosphere. The reaction was quenched with Water (400 mL) at room temperature. The aqueous layer was extracted with DCM (3×100 mL). The resulting mixture was concentrated under reduced pressure. The residue was purified by silica gel column chromatography, eluted with PE/EA (10:1) to afford Compound 125-1 (20 g, 85.77%) as a grey oil.

### Synthesis of Compound 125-2.

To a stirred solution of Compound 125-1 (20 g, 54.156 mmol, 1 equiv) and benzyl piperazine-1-carboxylate (14.31 g, 64.987 mmol, 1.2 equiv) in dioxane (200 mL) were added Cs₂CO₃ (52.94 g, 162.468 mmol, 3 equiv) and XPhos (2.58 g, 5.416 mmol, 0.1 equiv), XPhos Pd G3 (3.64 g, 5.420 mmol, 0.08 equiv) at room temperature. The resulting mixture was stirred for overnight at 100° C. under nitrogen atmosphere. The mixture was acidified to pH 5 with AcOH. The resulting mixture was concentrated under reduced pressure. The residue was purified by silica gel column chromatography, eluted with PE/EA (5:1) to afford Compound 125-2 (10.5 g, 34.30%) as a brown solid.

### Synthesis of Compound 125-3.

To a solution of Compound 125-2 (10.4 g, 20.446 mmol, 1 equiv) in 200 mL MeOH was added Pd/C (10%, 1 g) under nitrogen atmosphere. The mixture was hydrogenated at room temperature for overnight under hydrogen atmosphere using a hydrogen balloon, filtered through a Celite pad and concentrated under reduced pressure. This resulted in Compound 125-3 (7 g, 73.13%) as a brown solid.

### Synthesis of Compound 125-4.

To a stirred solution of Compound 125-3 (4 g, 10.680 mmol, 1 equiv) and Compound 39-3 (4.14 g, 12.816 mmol, 1.2 equiv) in dioxane (40 mL) were added Cs₂CO₃ (10.44 g, 32.040 mmol, 3 equiv) and {1,3-bis[2,6-bis(pentan-3-yl) phenyl]-4,5-dichloro-2,3-dihydro-1H-imidazol-2-yl} dichloro(2-methyl-1lambda4-pyridin-1-yl) palladium (0.45 g, 0.534 mmol, 0.05 equiv) at room temperature. The resulting mixture was stirred for overnight at 100° C. under nitrogen atmosphere. The mixture was acidified to pH 5 with AcOH. The resulting mixture was concentrated under reduced pressure. The residue was purified by silica gel column chromatography, eluted with DCM/MeOH (20:1) to afford Compound 125-4 (4 g, 54.65%) as a brown solid.

### Synthesis of Compound 125-5.

To a stirred solution of Compound 125-4 (4 g, 6.485 mmol, 1 equiv) and TFA (7 mL) in DCM (40 mL) for overnight at room temperature. The resulting mixture was concentrated under reduced pressure. The residue was purified by reversed-phase flash chromatography with the following conditions: column, C18 silica gel; mobile phase, MeCN in Water (0.1% FA), 30% to 50% gradient in 20 min; detector, UV 254 nm. This resulted in Compound 125-5 (3.4 g, 91.32%) as a brown solid.

### Synthesis of Compound 125-6.

To a stirred solution of Compound 125-5 (1.3 g, 2.516 mmol, 1 equiv) and Intermediate G (1.26 g, 2.768 mmol, 1.1 equiv) in DCM (13 mL) were added tetrakis(propan-2-yloxy) titanium (2.15 g, 7.548 mmol, 3 equiv) at room temperature. The resulting mixture was stirred for overnight at room temperature. To the above mixture was added NaBH(OAc)₃ (1.07 g, 5.032 mmol, 2 equiv) at room temperature. The resulting mixture was stirred for additional 3 h at room temperature. The mixture was acidified to pH 5 with water (10% AcOH). The resulting mixture was filtered; the filter cake was washed with water (3×10 mL). The filtrate was concentrated under reduced pressure. The residue was purified by reversed-phase flash chromatography with the following conditions: column, C18 silica gel; mobile phase, MeCN in Water (0.1% FA), 30% to 45% gradient in 20 min; detector, UV 254 nm. This resulted in Compound 125-6 (260 mg, 10.27%) as a yellow solid.

### Synthesis of Compound 125.

The PH-B-1935-6 (260 mg, 0.258 mmol, 1 equiv, 95%) was purified by Chiral Separation with the following conditions (Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 70% B to 70% B in 10 min; Wave Length: 220/254 nm; RT1 (min): 5.39; RT2 (min): 7.68; the first peak was product) to afford crude product (97.2 mg). The crude product (97.2 mg) was purified by reversed-phase flash chromatography with the following conditions: column, 40 g C18 silica gel; mobile phase, MeCN in Water (0.1% FA), 30% to 40% gradient in 10 min; detector, UV 254 nm to afford Compound 125-0 (70 mg, 27.21%) as a light yellow solid.

LC-MS-Compound 125: (ES, m/z): [M+H]⁺ 956

H-NMR-Compound 125: (400 MHz, DMSO-d6, δ ppm): 1.69-1.80 (m, 5H), 1.96-2.13 (m, 2H), 2.33-2.49 (m, 11H), 2.50-2.65 (m, 3H), 2.90-2.99 (m, 1H), 3.23-3.28 (m, 6H), 3.34-3.42 (m, 8H), 4.20-4.37 (m, 3H), 5.03-5.08 (m, 1H), 6.91-6.93 (m, 2H), 7.01 (s, 1H), 7.08-7.12 (m, 4H), 7.14-7.20 (m, 1H), 7.32 (s, 1H), 7.42-7.46 (m, 1H), 7.54-7.56 (m, 1H), 7.67-7.68 (d, 1H), 7.70-7.73 (m, 1H), 8.32 (s, 1H), 10.92 (s, 1H).

### Example 124. Compound 126

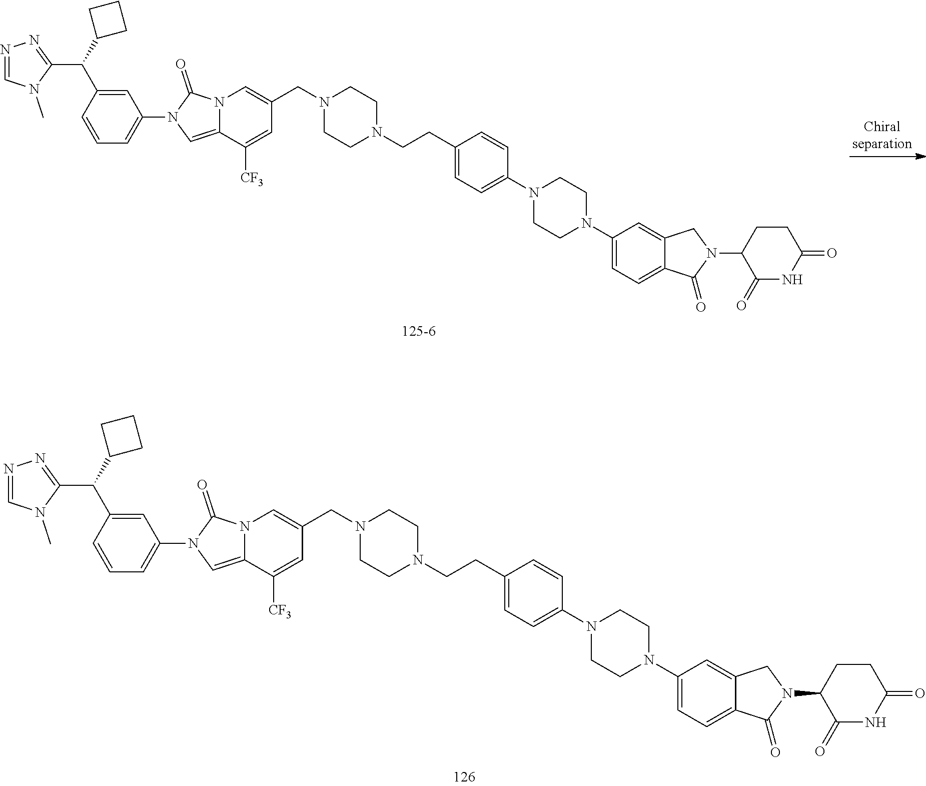 [SMILES: Cn1cnnc1[C@@H](c1cccc(-n2cc3c(C(F)(F)F)cc(CN4CCN(CCc5ccc(N6CCN(c7ccc8c(c7)CN(C7CCC(=O)NC7=O)C8=O)CC6)cc5)CC4)cn3c2=O)c1)C1CCC1 ; Cn1cnnc1[C@@H](c1cccc(-n2cc3c(C(F)(F)F)cc(CN4CCN(CCc5ccc(N6CCN(c7ccc8c(c7)CN([C@H]7CCC(=O)NC7=O)C8=O)CC6)cc5)CC4)cn3c2=O)c1)C1CCC1]

### Synthesis of Compound 126.

The Compound 125-6 (260 mg, 0.258 mmol, 1 equiv, 95%) purified by Chiral Separation with the following conditions (Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 70% B to 70% B in 10 min; Wave Length: 220/254 nm; RT1 (min): 5.39; RT2 (min): 7.68; the second peak was product) to afford crude product (94.3 mg). The crude product (94.3 mg) was purified by reversed-phase flash chromatography with the following conditions: column, 40 g C18 silica gel; mobile phase, MeCN in Water (0.1% FA), 30% to 40% gradient in 10 min; detector, UV 254 nm to afford Compound 126-0 (70.4 mg, 27.21%) as a light yellow solid.

LC-MS-Compound 126: (ES, m/z): [M+H]⁺ 956

H-NMR-Compound 126: (400 MHz, DMSO-d6, δ ppm): 1.69-1.83 (m, 5H), 1.95-2.09 (m, 2H), 2.33-2.45 (m, 11H), 2.50-2.67 (m, 3H), 2.90-2.99 (m, 1H), 3.24-3.28 (m, 6H), 3.33-3.42 (m, 8H), 4.20-4.37 (m, 3H), 5.03-5.08 (m, 1H), 6.91-6.93 (m, 2H), 7.01 (s, 1H), 7.08-7.12 (m, 4H), 7.14-7.20 (m, 1H), 7.32 (s, 1H), 7.42-7.46 (m, 1H), 7.54-7.56 (m, 1H), 7.67-7.68 (d, 1H), 7.70-7.73 (m, 1H), 8.32 (s, 1H), 10.92 (s, 1H).

### Example 125. Compound 127

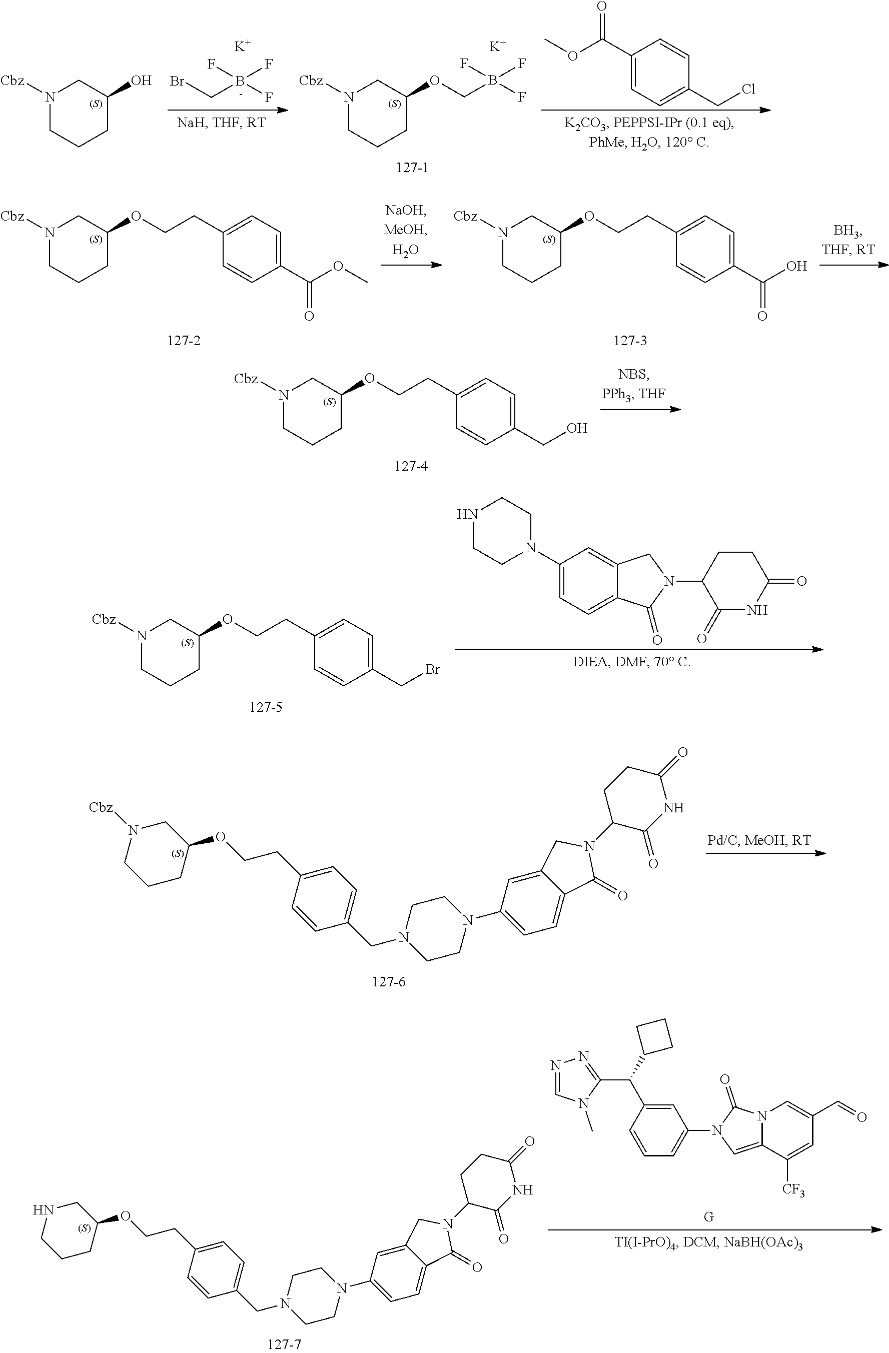

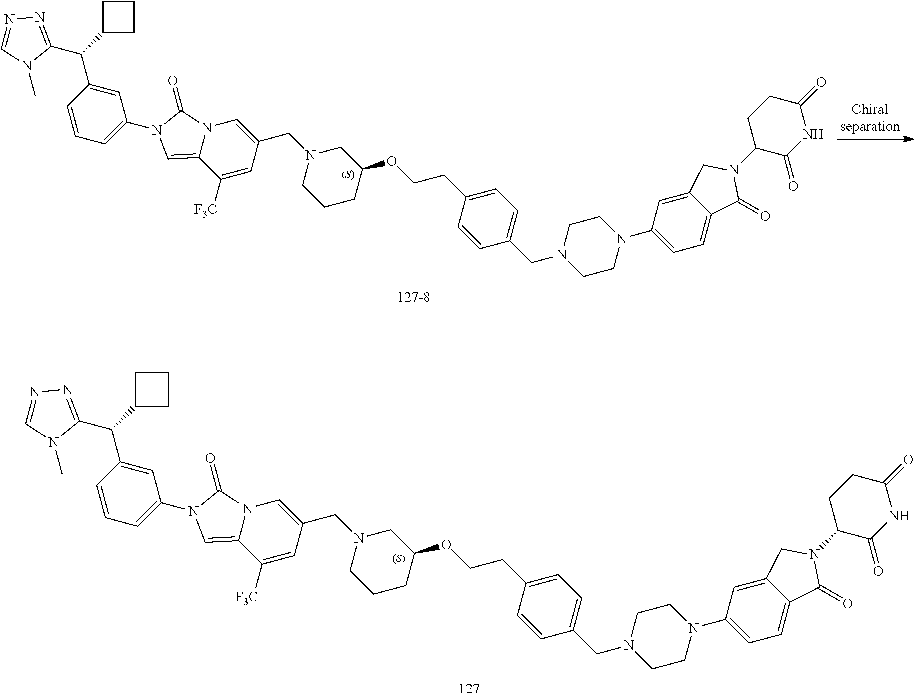

### Synthesis of Compound 127-1.

To a stirred mixture of NaH (2 g, 85.00 mmol, 2.0 equiv) and THF (100 mL) was added benzyl (3S)-3-hydroxypiperidine-1-carboxylate (10 g, 42.50 mmol, 1.0 equiv) at 0° C. under nitrogen atmosphere. The resulting mixture was stirred for 1 h at 0° C. under nitrogen atmosphere. To the above mixture was added potassium (bromomethyl) trifluoroboranuide (10 g, 50.98 mmol, 1.2 equiv) at 0° C. The resulting mixture was stirred for additional overnight at room temperature. The resulting mixture was concentrated under vacuum. The residue was purified by trituration with acetone (200 mL). The resulting mixture was filtered; the filter cake was washed with acetone (2×50 nmL). The filtrate was concentrated under reduced pressure. This resulted in Compound 127-1 (13 g, 97%) as a yellow oil.

### Synthesis of Compound 127-2.

Into a 500 mL 3-necked round-bottom flask were added Compound 127-1 (13 g, 41.12 mmol, 1.0 equiv), toluene (130 mL), H₂O (6 mL), methyl 4-(chloromethyl) benzoate (8.4 g, 45.64 mmol, 1.1 equiv) and K₂CO₃ (11.3 g, 82.25 mmol, 2.0 equiv) at room temperature. To the above mixture was added Pd-PEPPSI-IPentCl₂-methylpyridine (o-picoline (3.4 g, 4.18 mmol, 0.1 equiv) at room temperature. The resulting mixture was stirred for overnight at 120° C. under nitrogen atmosphere. The reaction was quenched by the addition of water (200 mL) at room temperature. The resulting mixture was extracted with EtOAc (3×200 mL). The combined organic layers were concentrated under reduced pressure. The residue was purified by silica gel column chromatography, eluted with CH₂Cl₂/MeOH (100:1) to afford Compound 127-2 (2.3 g, 13%) as a light yellow oil.

### Synthesis of Compound 127-3.

Into a 250 mL 3-necked round-bottom flask were added Compound 127-2 (4 g, 10.81 mmol, 1.0 equiv), MeOH (40 mL), H₂O (40 mL) and NaOH (0.6 g, 15.15 mmol, 1.4 equiv) at room temperature. The resulting mixture was stirred for overnight at room temperature under nitrogen atmosphere. The mixture was neutralized to pH 7 with saturated 1M HCl (aq.). The resulting mixture was extracted with CH₂Cl₂ (3×200 mL). The combined organic layers were concentrated under reduced pressure. The residue was purified by silica gel column chromatography, eluted with CH₂Cl₂/MeOH (40:1) to afford Compound 127-3 (3.5 g, 80%) as a light yellow oil.

### Synthesis of Compound 127-4.

Into a 100 mL 3-necked round-bottom flask were added Compound 127-3 (3.4 g, 8.86 mmol, 1.0 equiv) and THF (18 mL) at room temperature. To the above mixture was added BH₃-THF (178 mL, 17.74 mmol, 2.0 equiv) dropwise over 30 min at 0° C. The resulting mixture was stirred for additional 6 h at room temperature. The reaction was quenched by the addition of MeOH (100 mL) at room temperature. The mixture was basified to pH 7 with saturated NaHCO₃ (aq.). The resulting mixture was extracted with EtOAc (3×150 mL). The combined organic layers were concentrated under reduced pressure. The residue was purified by silica gel column chromatography, eluted with CH₂Cl₂/MeOH (50:1) to afford Compound 127-4 (2.5 g, 73%) as an off-white oil.

### Synthesis of Compound 127-5.

Into a 100 mL 3-necked round-bottom flask were added Compound 127-4 (2.5 g, 6.76 mmol, 1.0 equiv), THF (30 mL), PPh₃ (3.5 g, 13.53 mmol, 2.0 equiv) and NBS (2.4 g, 13.53 mmol, 2.0 equiv) at 0° C. The resulting mixture was stirred for 8 h at room temperature under nitrogen atmosphere. The reaction was quenched by the addition of sat. NH₄Cl (aq.) (100 mL) at room temperature. The resulting mixture was extracted with EtOAc (3×100 mL). The combined organic layers were concentrated under reduced pressure. The residue was purified by silica gel column chromatography, eluted with CH₂Cl₂/MeOH (80:1) to afford Compound 127-5 (2 g, 65%) as a light yellow oil.

### Synthesis of Compound 127-6.

Into a 100 mL 3-necked round-bottom flask were added Compound 127-5 (2 g, 4.62 mmol, 1.0 equiv), DMF (20 mL), Compound 67-6 (1.5 g, 4.62 mmol, 1.0 equiv) and DIEA (1.8 g, 13.88 mmol, 3.0 equiv) at room temperature. The resulting mixture was stirred for 3 h at 70° C. The reaction was quenched by the addition of water (20 mL) at room temperature. The resulting mixture was extracted with EtOAc (3×30 mL). The combined organic layers were concentrated under reduced pressure. The residue was purified by reversed-phase flash chromatography with the following conditions: column, C18 silica gel; mobile phase, MeCN in water (0.1% FA), 10% to 50% gradient in 30 min; detector, UV 254 nm. To afford Compound 127-6 (1.2 g, 36%) as a light yellow oil.

### Synthesis of Compound 127-7.

To a stirred solution of Compound 127-6 (1.2 g, 1.76 mmol, 1.0 equiv) in MeOH (30 mL) was added Pd/C (0.15 g) at room temperature under nitrogen atmosphere. The resulting mixture was stirred for 15 min at room temperature under hydrogen atmosphere. The resulting mixture was filtered; the filter cake was washed with MeOH (3×10 mL). The filtrate was concentrated under reduced pressure. The residue was purified by reversed-phase flash chromatography with the following conditions: column, C18 silica gel; mobile phase, MeCN in water (0.1% FA), 10% to 50% gradient in 10 min; detector, UV 254 nm. To afford Compound 127-7 (500 mg, 49%) as a light yellow oil.

### Synthesis of Compound 127-8.

To a stirred solution of Compound 127-7 (480 mg, 0.88 mmol, 1.0 equiv) and Intermediate G (481 mg, 1.06 mmol, 1.2 equiv) in DCM (5 mL) was added Ti(Oi-Pr)₄ (1 g, 3.52 mmol, 4.0 equiv) at room temperature. The resulting mixture was stirred for overnight at room temperature. To the above mixture was added NaBH(OAc)₃ (559 mg, 2.64 mmol, 3.0 equiv) at room temperature. The resulting mixture was stirred for additional 2 h at room temperature. The reaction was quenched by the addition of 10% AcOH (100 mL) at room temperature. The aqueous layer was extracted with EtOAc (3×60 mL). The aqueous phase was concentrated under reduced pressure. The residue was purified by reversed-phase flash chromatography with the following conditions: column, C18 silica gel; mobile phase, MeCN in water (0.1% FA), 10% to 50% gradient in 30 min; detector, UV 254 nm. The crude product (400 mg) was purified by Prep-HPLC with the following conditions (Column: Sunfire prep C18 column, 30*150 mm, 5 μm; Mobile Phase A: Water (0.1% FA), Mobile Phase B: ACN; Flow rate: 60 mL/min; Gradient: 12% B to 23% B in 8 min, 23% B; Wave Length: 254; 220 nm; RT1 (min): 7.98; Number of Runs: 0) to afford Compound 127-8 (220 mg, 25%) as a yellow solid.

### Synthesis of Compound 127.

The Compound 127-8 (220 mg) was purified by Prep-CHIRAL-HPLC with the following conditions Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 60% B to 60% B in 11 min; Wave Length: 220/254 nm; RT1 (min): 5.36; RT2 (min): 7.96; Sample Solvent: DCM:ACN=1:1—HPLC; Injection Volume: 0.8 mL; Number Of Runs: 4. The first peak was the product. This resulted in Compound 127 (74 mg, 34%) as a yellow solid.

LC-MS-Compound 127: (ES, m/z): [M+H]⁺ 985

H-NMR-Compound 127: (400 MHz, DMSO-d6, δ ppm): 1.08-1.12 (m, 1H), 1.31-1.40 (m, 1H), 1.78-2.07 (m, 12H), 2.47-2.55 (m, 4H), 2.63-2.73 (m, 3H), 2.75-2.77 (m, 2H), 2.85-2.89 (m, 3H), 3.20-3.26 (m, 6H), 3.42-3.45 (m, 5H), 3.60-3.62 (m, 2H), 4.17-4.33 (m, 3H), 5.01-5.06 (m, 1H), 7.00-7.03 (m, 3H), 7.18-7.21 (m, 5H), 7.31 (s, 1H), 7.41-7.45 (m, 1H), 7.49-7.51 (m, 1H), 7.66-7.74 (m, 3H), 8.32 (s, 1H), 10.94 (s, 1H).

### Example 126. Compound 128

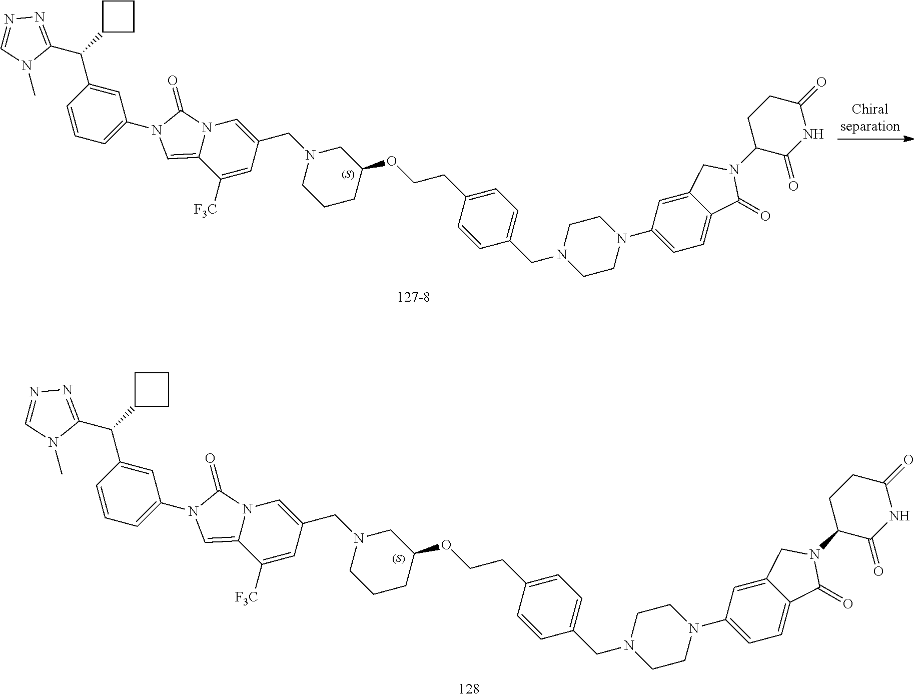

### Synthesis of Compound 128.

The Compound 127-8 (220 mg) was purified by Prep-CHIRAL-HPLC with the following conditions Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 60% B to 60% B in 11 min; Wave Length: 220/254 nm; RT1 (min): 5.36; RT2 (min): 7.96; Sample Solvent: DCM:ACN=1:1—HPLC; Injection Volume: 0.8 mL; Number of Runs: 4. The second peak was the product. This resulted in Compound 128 (73 mg, 33%) as a yellow solid.

LC-MS-Compound 128: (ES, m/z): [M+H]⁺ 985

H-NMR-Compound 128: (400 MHz, DMSO-d6, δ ppm): 1.08-1.12 (m, 1H), 1.31-1.40 (m, 1H), 1.78-2.07 (m, 12H), 2.47-2.55 (m, 4H), 2.63-2.73 (m, 3H), 2.75-2.77 (m, 2H), 2.85-2.89 (m, 3H), 3.20-3.26 (m, 6H), 3.42-3.45 (m, 5H), 3.60-3.62 (m, 2H), 4.17-4.33 (m, 3H), 5.01-5.06 (m, 1H), 7.00-7.03 (m, 3H), 7.18-7.21 (m, 5H), 7.31 (s, 1H), 7.41-7.45 (m, 1H), 7.49-7.51 (m, 1H), 7.66-7.74 (m, 3H), 8.32 (s, 1H), 10.94 (s, 1H).

### Example 127. Compound 129

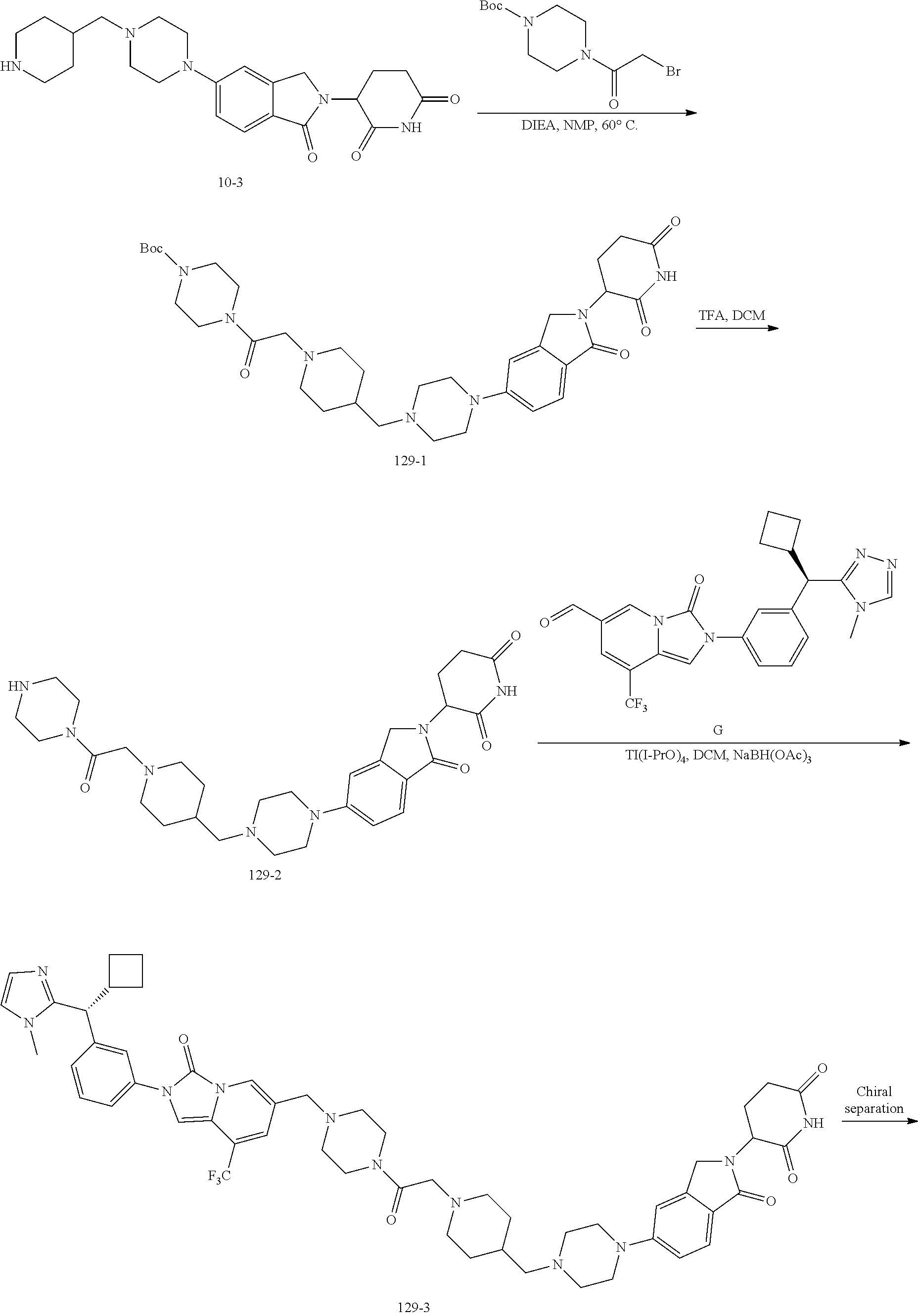

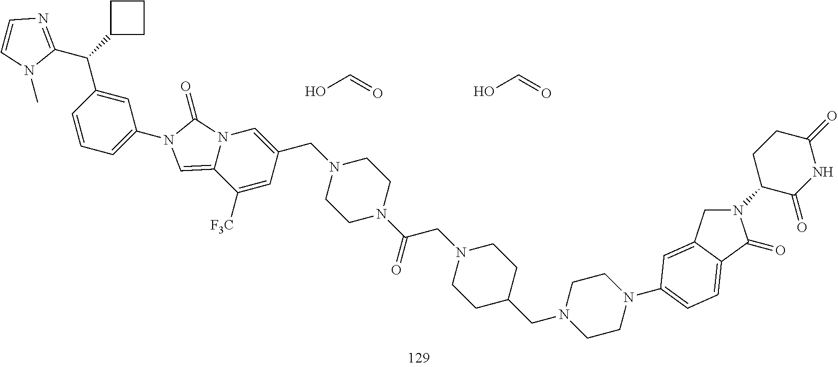 [SMILES: Cn1ccnc1[C@@H](c1cccc(-n2cc3c(C(F)(F)F)cc(CN4CCN(C(=O)CN5CCC(CN6CCN(c7ccc8c(c7)CN([C@@H]7CCC(=O)NC7=O)C8=O)CC6)CC5)CC4)cn3c2=O)c1)C1CCC1 ; O=CO ; O=CO]

### Synthesis of Compound 129-1.

To a stirred solution of Compound 10-3 (3.0 g, 7.05 mmol, 1.0 equiv) and tert-butyl 4-(2-bromoacetyl)piperazine-1-carboxylate (2.1 g, 7.05 mmol, 1.0 equiv) in NMP (30 mL) were added DIEA (2.7 g, 21.15 mmol, 3.0 equiv) at room temperature under nitrogen atmosphere. The resulting mixture was stirred for overnight at 60° C. under nitrogen atmosphere. The residue was purified by reversed-phase flash chromatography with the following conditions: column, C18 silica gel; mobile phase, MeCN in water (0.1% FA), 10% to 50% gradient in 30 min; detector, UV 254 nm. This resulted in Compound 129-1 (2.0 g, 43%) as a white solid.

### Synthesis of Compound 129-2.

To a stirred solution of Compound 129-1 (1.9 g, 3.04 mmol, 1.0 equiv) in DCM (5 mL) was added TFA (5 mL) at room temperature under nitrogen atmosphere. The resulting mixture was stirred for overnight at room temperature under nitrogen atmosphere. The reaction was quenched by the addition of water (60 mL) at room temperature. The aqueous layer was extracted with CH₂Cl₂ (3×30 mL). The resulting mixture was concentrated under reduced pressure. This resulted in Compound 129-2 (1.6 g, 94%) as a white solid.

### Synthesis of Compound 129-3.

To a stirred solution of Compound 129-2 (780 mg, 1.41 mmol, 1.0 equiv) and Intermediate G (773 mg, 1.69 mmol, 1.2 equiv) in DCM (8 mL) were added TEA (143 Ing, 1.41 mmol, 1.0 equiv) and Ti(Oi-Pr)₄ (1607 mg, 5.66 mmol, 4.0 equiv) at room temperature under nitrogen atmosphere. The resulting mixture was stirred for overnight at room temperature under nitrogen atmosphere. To the above mixture was added NaBH(OAc)₃ (899 mg, 4.24 mmol, 3.0 equiv) at room temperature. The resulting mixture was stirred for additional 4 h at room temperature. The reaction was quenched by the addition of water/HOAc=10:1 (80 mL) at room temperature. The aqueous layer was extracted with CH₂Cl₂/MeOH 5:1 (3×50 mL). The resulting mixture was concentrated under reduced pressure. The residue was purified by reversed-phase flash chromatography with the following conditions: column, C18 silica gel; mobile phase, MeCN in water (0.1% FA), 10% to 50% gradient in 30 min; detector, UV 254 nm. This resulted in Compound 129-3 (440 mg, 30%) as a yellow solid.

### Synthesis of Compound 129.

The Compound 129 (490 mg, 0.49 mmol, 1.0 equiv) was purified by Prep-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 25% B to 25% B in 25 min; Wave Length: 220/254 nm; RT1 (min): 11.34; RT2 (min): 16.57; the first peak was product) to afford crude product. The residue was purified by reversed-phase flash chromatography with the following conditions: column, C18 silica gel; mobile phase, MeCN in water (0.1% FA), 10% to 50% gradient in 30 min; detector, UV 254 nm. This resulted in Compound 129 (183 mg, 36%) as a yellow solid.

LCMS-Compound 129: (ES, m/z): [M−2×HCOOH+H]⁺ 991

NMR-Compound 129: (400 MHz, DMSO-d6, δ ppm): 1.09-1.11 (m, 2H), 1.48-1.53 (m, 1H), 1.67-1.79 (m, 7H), 1.96-2.16 (m, 6H), 2.34-2.45 (m, 10H), 2.50-2.51 (m, 1H), 2.79-2.90 (m, 3H), 3.15-3.18 (m, 2H), 3.26-3.40 (m, 9H), 3.42-3.55 (m, 4H), 4.17-4.35 (m, 3H), 5.03-5.07 (m, 1H), 7.03-7.06 (d, 3H), 7.18-7.21 (d, 1H), 7.33 (s, 1H), 7.42-7.46 (t, 1H), 7.51-7.53 (d, 1H), 7.69-7.74 (m, 3H), 8.16 (s, 1H), 8.33 (s, 1H), 10.98 (s, 1H).

### Example 128. Compound 130

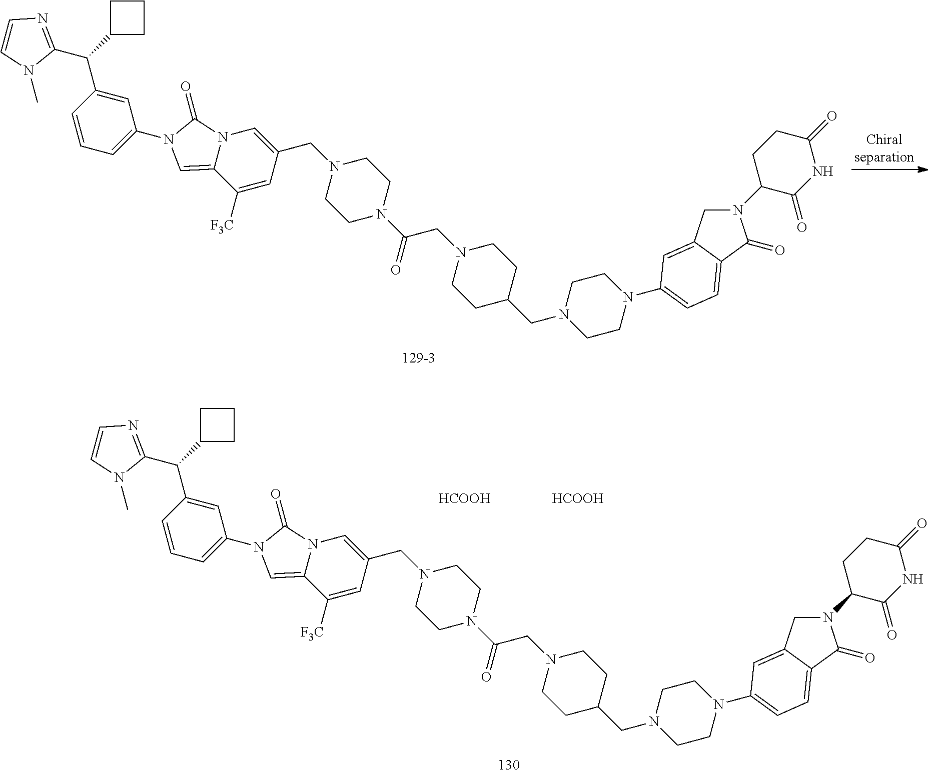 [SMILES: Cn1ccnc1[C@@H](c1cccc(-n2cc3c(C(F)(F)F)cc(CN4CCN(C(=O)CN5CCC(CN6CCN(c7ccc8c(c7)CN(C7CCC(=O)NC7=O)C8=O)CC6)CC5)CC4)cn3c2=O)c1)C1CCC1 ; Cn1ccnc1[C@@H](c1cccc(-n2cc3c(C(F)(F)F)cc(CN4CCN(C(=O)CN5CCC(CN6CCN(c7ccc8c(c7)CN([C@H]7CCC(=O)NC7=O)C8=O)CC6)CC5)CC4)cn3c2=O)c1)C1CCC1 ; "HCOOH" ; "HCOOH"]

### Synthesis of Compound 130.

The Compound 129-3 (490 mg, 0.49 mmol, 1.0 equiv) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 25% B to 25% B in 25 min; Wave Length: 220/254 nm; RT1 (min): 11.34; RT2 (min): 16.57; Sample Solvent: DCM:ACN=1:1; the second peak was product) to afford crude product. The residue was purified by reversed-phase flash chromatography with the following conditions: column, C18 silica gel; mobile phase, MeCN in water (0.1% FA), 10% to 50% gradient in 30 min; detector, UV 254 nm. This resulted in Compound 130 (176.8 mg, 32.45%) as a yellow solid.

LCMS-Compound 130: (ES, m/z): [M−2×HCOOH+H]⁺ 991

NMR-Compound 130: (400 MHz, DMSO-d6, δ ppm): 1.09-1.12 (m, 2H), 1.49-1.53 (m, 1H), 1.67-1.80 (m, 7H), 1.96-2.16 (m, 6H), 2.35-2.50 (m, 10H), 2.50-2.51 (m, 1H), 2.81-2.90 (m, 3H), 3.17-3.26 (m, 2H), 3.34-3.40 (m, 9H), 3.42-3.55 (m, 4H), 4.18-4.35 (m, 3H), 5.03-5.07 (m, 1H), 7.03-7.06 (d, 3H), 7.18-7.21 (d, 1H), 7.33 (s, 1H), 7.42-7.46 (t, 1H), 7.53-7.69 (d, 1H), 7.71-7.74 (m, 3H), 8.15 (s, 1H), 8.33 (s, 1H), 10.99 (s, 1H).

### Example 129. Compound 131

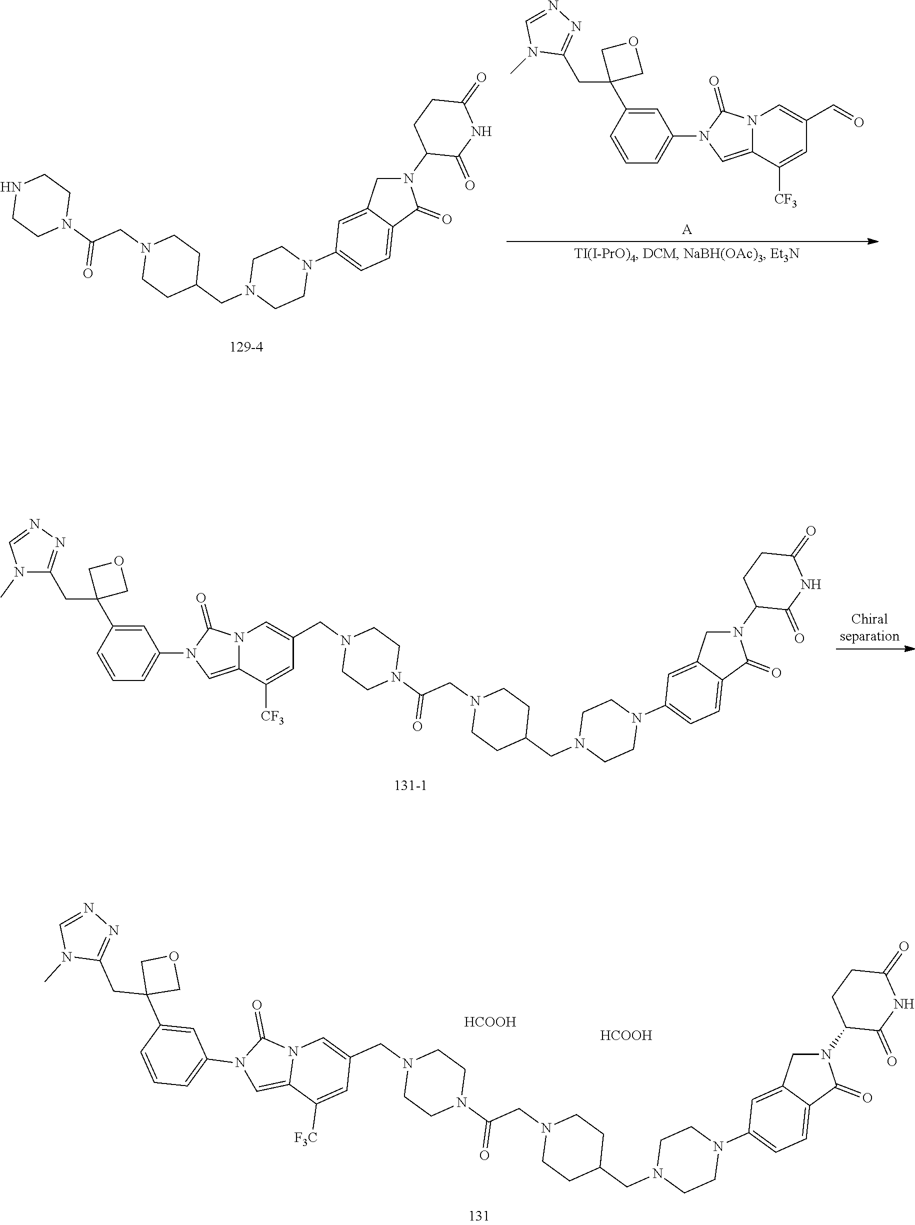

### Synthesis of Compound 131-1.

To a stirred solution of Compound 129-4 (780 mg, 1.41 mmol, 1.0 equiv) and Intermediate A (646 mg, 1.41 mmol, 1.0 equiv) in DCM (8 mL) were added Et₃N (143 mg, 1.41 mmol, 1.0 equiv) and Ti(Oi-Pr)₄ (1607 mg, 5.66 mmol, 4.0 equiv) at room temperature under nitrogen atmosphere. The resulting mixture was stirred for overnight at room temperature. To the above mixture was added NaBH(OAc)₃ (899 mg, 4.24 mmol, 3.0 equiv) at room temperature under nitrogen atmosphere. The resulting mixture was stirred for additional 4 h at room temperature. The reaction was quenched by the addition of water/AcOH=10:1 (80 mL) at room temperature. The aqueous layer was extracted with CH₂Cl₂/MeOH=5:1 (3×50 mL). The resulting mixture was concentrated under reduced pressure. The residue was purified by reversed-phase flash chromatography with the following conditions: column, C18 silica gel; mobile phase, MeCN in water (0.1% FA), 10% to 50% gradient in 30 min; detector, UV 254 nm. This resulted in Compound 131-1 (430 mg, 29%) as a yellow solid.

### Synthesis of Compound 131.

The Compound 131-1 (490 mg, 0.49 mmol, 1.0 equiv) was purified by Prep-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 90% B to 90% B in 38 min; Wave Length: 220/254 nm; RT1 (min): 15.10; RT2 (min): 24.94; Sample Solvent: DCM:ACN=1:1; Injection Volume: 0.9 mL; the first peak was product) to afford crude product. The residue was purified by reversed-phase flash chromatography with the following conditions: column, C18 silica gel; mobile phase, MeCN in water (0.1% FA), 10% to 50% gradient in 30 min; detector, UV 254 nm. This resulted in Compound 131 (127.6 mg, 24%) as a yellow solid.

LCMS-Compound 131: (ES, m/z): [M−2×HCOOH+H]⁺ 497

NMR-Compound 131: (400 MHz, DMSO-d6, δ ppm): 1.09-1.12 (m, 2H), 1.49-1.59 (m, 1H), 1.68-1.71 (m, 3H), 1.98-2.05 (m, 2H), 2.15-2.17 (m, 3H), 2.35-2.46 (m, 5H), 2.50-2.60 (m, 1H), 2.81-2.87 (m, 3H), 2.90-2.95 (m, 1H), 2.97 (s, 3H), 3.17-3.27 (m, 1H), 3.33-3.45 (m, 6H), 3.49-3.53 (m, 7H), 4.17-4.22 (d, 1H), 4.30-4.34 (d, 1H), 4.92-5.02 (m, 4H), 5.03-5.07 (m, 1H), 6.89-6.91 (d, 1H), 7.03-7.06 (m, 3H), 7.32-7.41 (m, 3H), 7.50-7.53 (d, 1H), 7.74-7.77 (m, 2H), 8.15-8.20 (m, 3H), 10.94 (s, 1H).

### Example 130. Compound 132

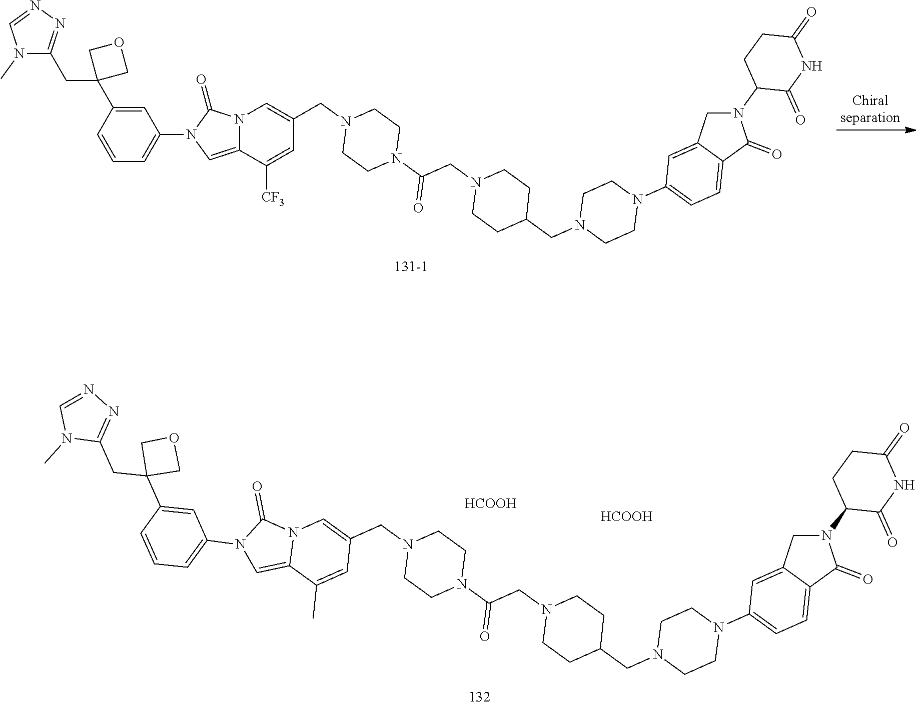 [SMILES: Cn1cnnc1CC1(c2cccc(-n3cc4c(C(F)(F)F)cc(CN5CCN(C(=O)CN6CCC(CN7CCN(c8ccc9c(c8)CN(C8CCC(=O)NC8=O)C9=O)CC7)CC6)CC5)cn4c3=O)c2)COC1 ; Cc1cc(CN2CCN(C(=O)CN3CCC(CN4CCN(c5ccc6c(c5)CN([C@H]5CCC(=O)NC5=O)C6=O)CC4)CC3)CC2)cn2c(=O)n(-c3cccc(C4(Cc5nncn5C)COC4)c3)cc12 ; "HCOOH" ; "HCOOH"]

### Synthesis of Compound 132.

The Compound 131-1 (490 mg, 0.49 mmol, 1.0 equiv)) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 90% B to 90% B in 38 min; Wave Length: 220/254 nm; RT1 (min): 15.10; RT2 (min): 24.94; the second peak was product) to afford crude product. The residue was purified by reversed-phase flash chromatography with the following conditions: column, C18 silica gel; mobile phase, MeCN in water (0.1% FA), 10% to 50% gradient in 30 min; detector, UV 254 nm. This resulted in Compound 132 (102.3 mg, 20%) as a yellow solid.

LCMS-Compound 132: (ES, m/z): [M−2×HCOOH+H]⁺ 497

NMR-Compound 132: (400 MHz, DMSO-d6, δ ppm): 1.09-1.12 (m, 2H), 1.49-1.59 (m, 1H), 1.68-1.71 (m, 2H), 1.97-2.04 (m, 3H), 2.15-2.17 (m, 2H), 2.35-2.38 (m, 3H), 2.46-2.50 (m, 5H), 2.56-2.60 (m, 1H), 2.81-2.97 (m, 7H), 3.15-3.18 (m, 2H), 3.26-3.33 (m, 5H), 3.35-3.37 (m, 3H), 3.45-3.53 (m, 4H), 4.18-4.22 (d, 1H), 4.30-4.35 (d, 1H), 4.90-5.07 (m, 5H), 6.89-6.91 (d, 1H), 7.03-7.06 (m, 3H), 7.32-7.38 (t, 1H), 7.40-7.41 (m, 2H), 7.50-7.53 (d, 1H), 7.71-7.77 (m, 2H), 8.15-8.20 (m, 3H), 10.94 (s, 1H).

### Example 131. Compound 133

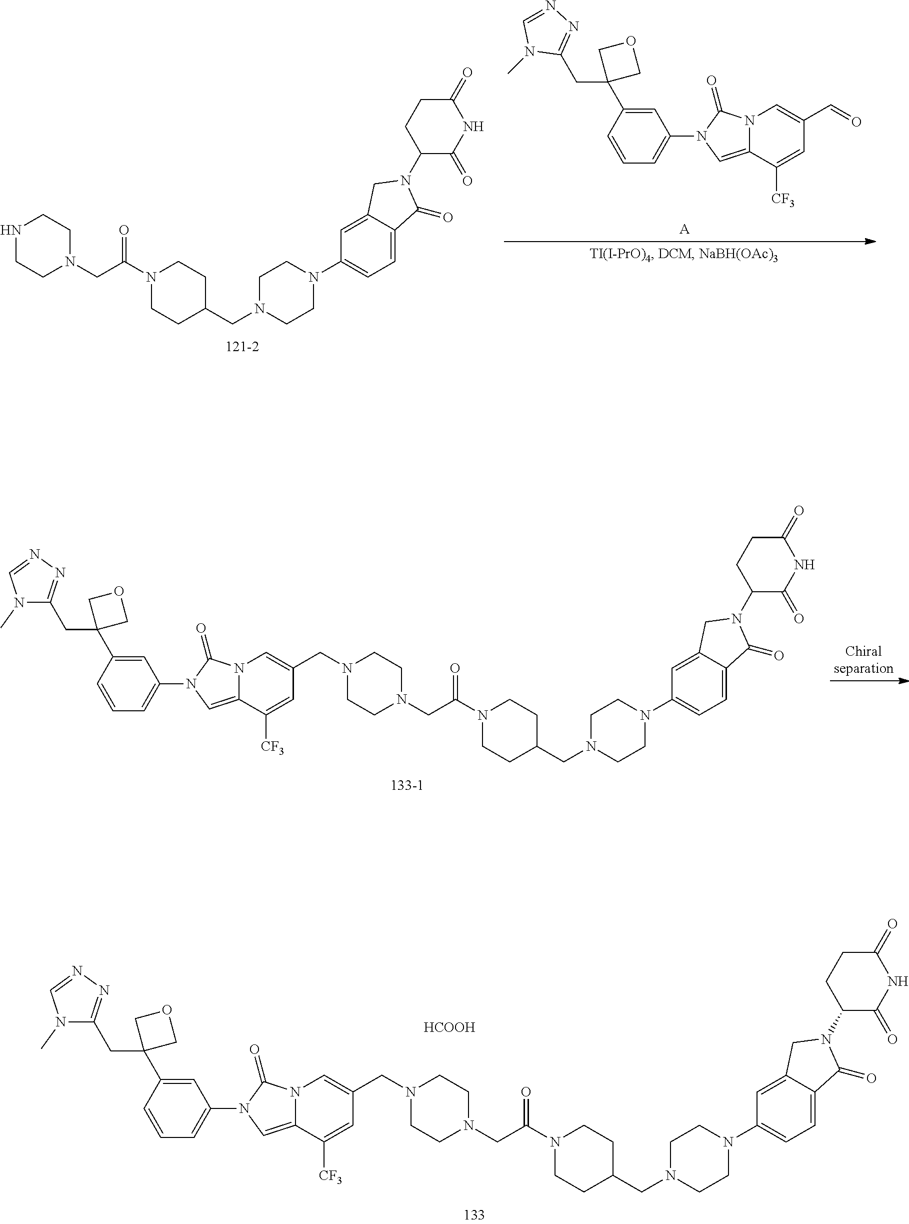

### Synthesis of Compound 133-1.

To a stirred solution of Compound 121-2 (680 mg, 1.23 mmol, 1.0 equiv) and Intermediate A (676 mg, 1.48 mmol, 1.2 equiv) in DCM (7 mL) was added Ti(Oi-Pr)₄ (1401 mg, 4.93 mmol, 4.0 equiv) at room temperature. The resulting mixture was stirred for overnight at room temperature. To the above mixture was added NaBH(OAc)₃ (783 mg, 3.69 mmol, 3.0 equiv) over 2 h at room temperature. The resulting mixture was stirred for additional 2 h at room temperature. The reaction was quenched by the addition of 10% AcOH (100 mL) at room temperature. The aqueous layer was extracted with EtOAc (3×60 mL). The aqueous phase was concentrated under reduced pressure. The residue was purified by reversed-phase flash chromatography with the following conditions: column, C18 silica gel; mobile phase, MeCN in water (0.1% FA), 10% to 50% gradient in 30 min; detector, UV 254 nm. The crude product (400 mg) was purified by Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 6% B to 20% B in 7 min, 20% B; Wave Length: 254; 220 nm; RT1 (min): 6.3; Number of Runs: 0) to afford Compound 133-1 (0.200 mg, 16%) as a yellow solid.

### Synthesis of Compound 133.

The Compound 133-1 (200 mg) was purified by Prep-CHIRAL-HPLC with the following conditions Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 90% B to 90% B in 30 min; Wave Length: 220/254 nm; RT1 (min): 16.50; RT2 (min): 23.82; Sample Solvent: DCM:ACN=1:1—HPLC; Injection Volume: 0.6 mL; Number Of Runs: 6. The first peak was the product. The residue was purified by reversed-phase flash chromatography with the following conditions: column, C18 silica gel; mobile phase, MeCN in water (0.1% FA), 10% to 50% gradient in 30 min; detector, UV 254 nm. This resulted in Compound 133 (96 mg, 46%) as a yellow solid.

LC-MS-Compound 133: (ES, m/z): [M-COOH]⁺ 993

H-NMR-Compound 133: (400 MHz, DMSO-d6, δ ppm): 0.85.95 (m, 1H), 1.02-1.10 (m, 1H), 1.72-1.80 (m, 3H), 1.90-1.95 (m, 1H), 2.17-2.18 (m, 2H), 2.32-2.43 (m, 9H), 2.54-2.60 (m, 2H), 2.89-2.96 (m, 7H), 3.29-3.37 (m, 9H), 3.53 (s, 3H), 3.95-4.00 (m, 1H), 4.18-4.22 (m, 1H), 4.30-4.35 (m, 1H), 4.90-4.95 (m, 4H), 5.03-5.07 (m, 1H), 6.87-6.89 (m, 1H), 7.00 (s, 1H), 7.04-7.06 (m, 2H), 7.32 (s, 1H), 7.37-7.41 (m, 2H), 7.51-7.53 (m, 1H), 7.68 (s, 1H), 7.74-7.75 (d, 1H), 8.14 (s, 1H), 8.20 (s, 1H), 10.95 (s, 1H).

### Example 132. Compound 134

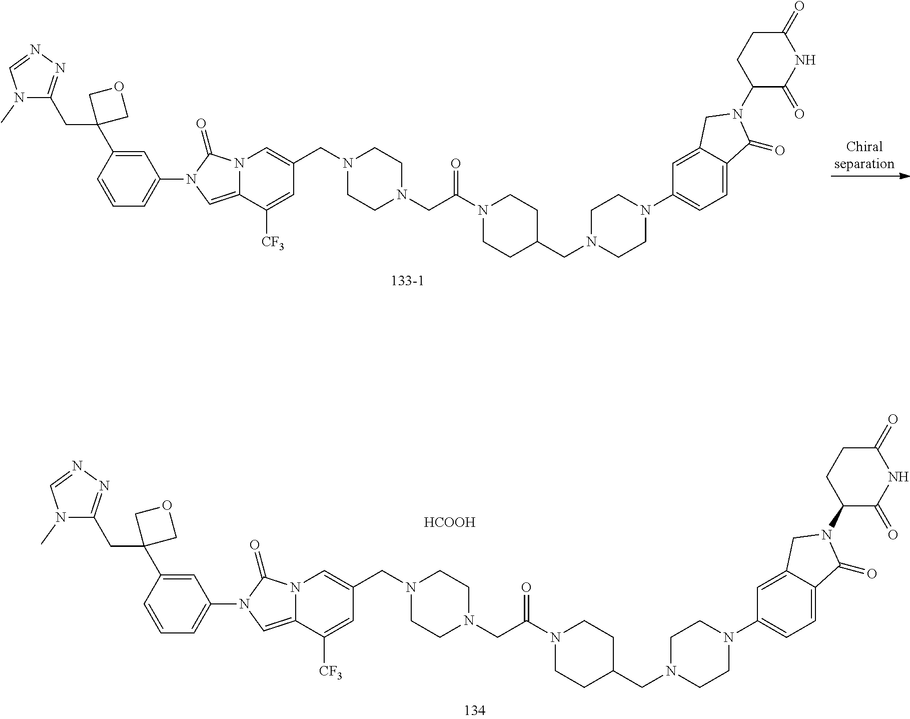 [SMILES: Cn1cnnc1CC1(c2cccc(-n3cc4c(C(F)(F)F)cc(CN5CCN(CC(=O)N6CCC(CN7CCN(c8ccc9c(c8)CN(C8CCC(=O)NC8=O)C9=O)CC7)CC6)CC5)cn4c3=O)c2)COC1 ; Cn1cnnc1CC1(c2cccc(-n3cc4c(C(F)(F)F)cc(CN5CCN(CC(=O)N6CCC(CN7CCN(c8ccc9c(c8)CN([C@H]8CCC(=O)NC8=O)C9=O)CC7)CC6)CC5)cn4c3=O)c2)COC1 ; "HCOOH"]

### Synthesis of Compound 134.

The Compound 133-1 (200 Ing) was purified by Prep-CHIRAL-HPLC with the following conditions Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 90% B to 90% B in 30 min; Wave Length: 220/254 nm; RT1 (min): 16.50; RT2 (min): 23.82; Sample Solvent: DCM:ACN=1:1—HPLC; Injection Volume: 0.6 mL; Number Of Runs: 6. The second peak was the product. The residue was purified by reversed-phase flash chromatography with the following conditions: column, C18 silica gel; mobile phase, MeCN in water (0.1% FA), 10% to 50% gradient in 30 min; detector, UV 254 nm. This resulted in Compound 134 (90 mg, 43%) as a yellow solid.

LC-MS-Compound 134: (ES, m/z): [M-COOH]⁺ 993

H-NMR-Compound 134: (400 MHz, DMSO-d6, δ ppm): 0.85.95 (m, 1H), 1.02-1.10 (m, 1H), 1.72-1.80 (m, 3H), 1.90-1.95 (m, 1H), 2.17-2.18 (m, 2H), 2.32-2.43 (m, 9H), 2.54-2.60 (m, 2H), 2.89-2.96 (m, 7H), 3.29-3.37 (m, 9H), 3.53 (s, 3H), 3.95-4.00 (m, 1H), 4.18-4.22 (m, 1H), 4.30-4.35 (m, 1H), 4.90-4.95 (m, 4H), 5.03-5.07 (m, 1H), 6.87-6.89 (m, 1H), 7.00 (s, 1H), 7.04-7.06 (m, 2H), 7.32 (s, 1H), 7.37-7.41 (m, 2H), 7.51-7.53 (m, 1H), 7.68 (s, 1H), 7.74-7.75 (d, 1H), 8.14 (s, 1H), 8.20 (s, 1H), 10.95 (s, 1H).

### Example 133. Compound 135

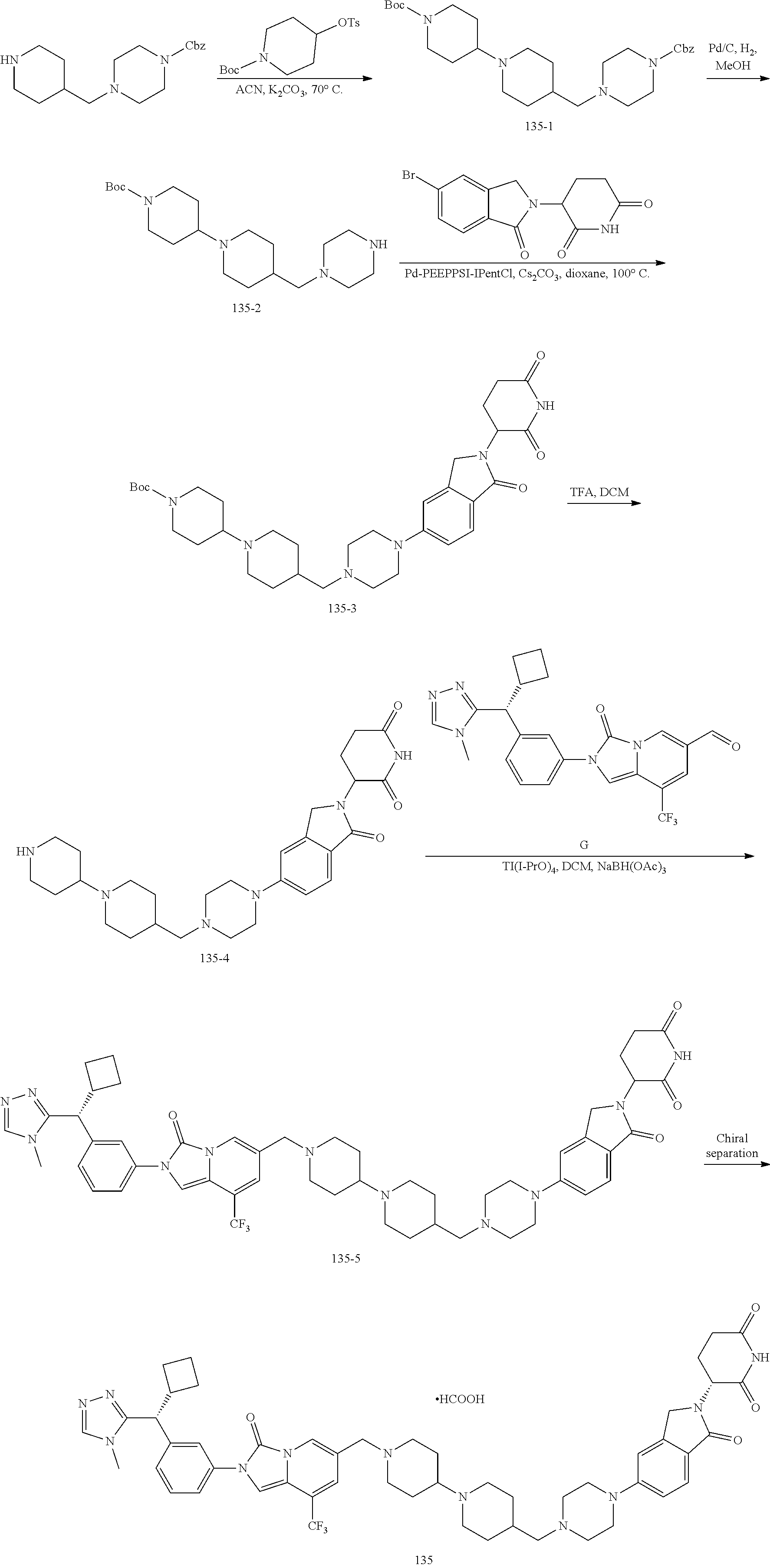

### Synthesis of Compound 135-1.

To a stirred solution of benzyl 4-(piperidin-4-ylmethyl) piperazine-1-carboxylate (20 g, 63.005 mmol, 1 equiv) and tert-butyl 4-[(4-methylbenzenesulfonyl oxy]piperidine-1-carboxylate (33.59 g, 94.508 mmol, 1.5 equiv) in ACN (200 mL) was added K₂CO₃ (17.42 g, 126.010 mmol, 2 equiv). The resulting mixture was stirred for overnight at 70° C. The reaction was quenched with sat. NH₄Cl (aq.) (500 mL) at room temperature. The resulting mixture was extracted with EtOAc (3×200 mL). The combined organic layers were concentrated under reduced pressure. The residue was purified by silica gel column chromatography, eluted with PE/EA (10:1) to afford Compound 135-1 (15 g, 43.75%) as a yellow solid.

### Synthesis of Compound 135-2.

To a stirred solution of Compound 135-1 (15 g, 29.959 mmol, 1 equiv) in MeOH (450 mL) was added Pd/C (1.5 g, 1.410 mmol, 0.05 equiv, 10%) in portions at room temperature under hydrogen atmosphere. The resulting mixture was stirred for additional 3 h at room temperature. The resulting mixture was filtered; the filter cake was washed with MeOH (3×20 ml). The filtrate was concentrated under reduced pressure. This resulted in Compound 135-2 (10 g, 85.60%) as a brown solid.

### Synthesis of Compound 135-3.

To a stirred solution of 3-(5-bromo-1-oxo-3H-isoindol-2-yl)piperidine-2,6-dione (7.05 g, 21.824 mmol, 1 equiv), Compound 135-2 (9.17 g, 28.372 mmol, 1.3 equiv) in 1,4-dioxane 100 ml were added Cs₂CO₃ (21.33 g, 65.475 mmol, 3 equiv) and Pd-PEPPSI-IPentCl (1.84 g, 2.183 mmol, 0.1 equiv) at room temperature under nitrogen atmosphere. The resulting mixture was stirred for overnight at 100° C. under nitrogen atmosphere. The reaction was quenched by the addition of 10% AcOH (aq.) (500 mL) at room temperature. The resulting mixture was concentrated under reduced pressure. The residue was purified by silica gel column chromatography, eluted with DCM/MeOH (20:1) to afford Compound 135-3 (5 g, 3.39%) as a grey solid.

### Synthesis of Compound 135-4.

A solution of Compound 135-3 (5 g, 8.213 mmol, 1 equiv) and TFA (12.5 mL) in DCM (37.5 mL) was stirred for overnight at mom temperature. The resulting mixture was concentrated under reduced pressure. The residue was purified by reverse flash chromatography with the following conditions: column, C18 silica gel; mobile phase, MeCN in water (FA 0.1%), 40% to 60% gradient in 18 min; detector, UV 220 nm. This resulted in Compound 135-4 (3 g, 64.63%) as a brown oil.

### Synthesis of Compound 135-5.

To a stirred solution of Compound 135-4 (3 g, 5.898 mmol, 1 equiv) and Intermediate G (2.69 g, 5.898 mmol, 1 equiv) in DCE (30 mL) was added Ti(Oi-Pr)₄ (8.38 g, 29.490 mmol, 5 equiv). The resulting mixture was stirred for overnight at room temperature. To the above mixture was added NaBH(OAc)₃ (3.75 g, 17.694 mmol, 3 equiv). The resulting mixture was stirred for additional 4 h at room temperature. The reaction was quenched by the addition of 10% HOAc (200 mL). The resulting mixture was extracted with DCM/MeOH=8/1 (3×100 mL). The combined organic layers were concentrated under reduced pressure. The crude product was purified by Prep-HPLC with the following conditions (Column: SunFire C18 OBD Prep Column, 19*250 mm, 5 μm; Mobile Phase A: water (0.1% FA), Mobile Phase B: ACN; Flow rate: 25 mL/min; Gradient: 8% B to 23% B in 8 min, 23% B; Wave Length: 254; 220 nm; RT1 (min): 7.08) to afford Compound 135-5 (160 mg, 2.75%) as a yellow solid.

### Synthesis of Compound 135.

The Compound 135-5 (160 mg) was purified by Prep-Chiral-HPLC with the following conditions (Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 70% B to 70% B in 13 min; Wave Length: 220/254 nm; RT1 (min): 5.77; RT2 (min): 10.11. The first peak was the product. Sample Solvent: DCM:ACN=1:1—HPLC; Injection Volume: 1 mL; Number of Runs: 4) to afford crude product. The crude product was purified by reversed-phase flash chromatography with the following conditions: column, 40 g C18 silica gel; mobile phase, MeCN in water (0.1% FA), 40% to 65% gradient in 20 min; detector, UV 254 nm to afford Compound 135 (9.7 mg, 5.71%) as a yellow solid.

LC-MS-Compound 135: (ES, m/z): [[M−HCOOH]+H]⁺ 949

H-NMR-Compound 135: 1H NMR (400 MHz, DMSO-d6) δ1.08-1.11 (m, 2H), δ1.44-1.47 (m, 3H), δ1.70-1.80 (m, 9H), δ1.93-1.97 (m, 3H), δ2.07-2.23 (m, 5H), δ2.30-2.34 (m, 2H), δ2.47-2.49 (m, 4H), δ2.50-2.60 (m, 2H), δ2.85-2.94 (m, 5H), δ3.21-3.26 (m, 6H), 63.43 (s, 3H), δ4.17-4.34 (m, 3H), 65.03-5.05 (m, 1H), δ7.00-7.06 (m, 3H), δ7.18-7.20 (m, 1H), 67.31 (s, 1H), δ7.42-7.45 (t, 1H), δ7.50-7.52 (d, 1H), δ7.65-7.73 (m, 3H), δ8.32 (s, 1H), δ10.94 (s, 1H).

### Example 134. Compound 136

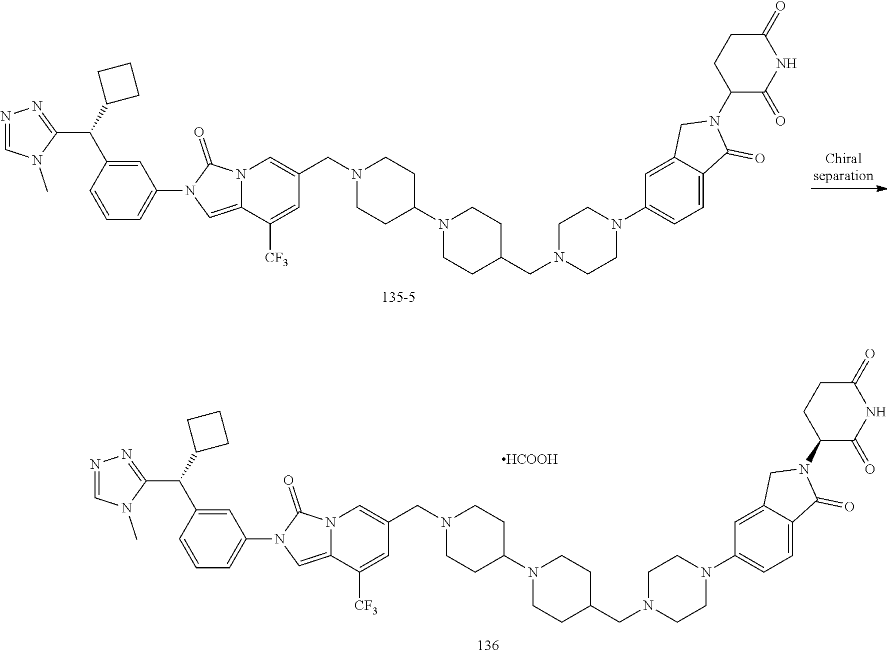 [SMILES: Cn1cnnc1[C@@H](c1cccc(-n2cc3c(C(F)(F)F)cc(CN4CCC(N5CCC(CN6CCN(c7ccc8c(c7)CN(C7CCC(=O)NC7=O)C8=O)CC6)CC5)CC4)cn3c2=O)c1)C1CCC1 ; Cn1cnnc1[C@@H](c1cccc(-n2cc3c(C(F)(F)F)cc(CN4CCC(N5CCC(CN6CCN(c7ccc8c(c7)CN([C@H]7CCC(=O)NC7=O)C8=O)CC6)CC5)CC4)cn3c2=O)c1)C1CCC1 ; "·HCOOH"]

### Synthesis of Compound 136.

The Compound 135-5 (160 mg) was purified by Prep-Chiral-HPLC with the following conditions (Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 70% B to 70% B in 13 min; Wave Length: 220/254 nm; RT1 (min): 5.77; RT2 (min): 10.11. The second peak was the product. Sample Solvent: DCM:ACN=1:1—HPLC; Injection Volume: 1 mL; Number of Runs: 4) to afford crude product. The crude product was purified by reversed-phase flash chromatography with the following conditions: column, 40 g C18 silica gel; mobile phase, MeCN in water (0.1% FA), 40% to 60% gradient in 20 min; detector, UV 254 nm to afford Compound 118 (18.0 mg, 10.59%) as a yellow solid.

LC-MS-Compound 136: (ES, m/z): [[M−HCOOH]+H]⁺ 949

H-NMR-Compound 136: 1H NMR (400 MHz, DMSO-d6) δ1.08-1.11 (m, 2H), δ1.44-1.47 (m, 3H), δ1.70-1.80 (m, 10H), δ1.93-1.97 (m, 3H), δ2.07-2.15 (m, 1H), δ2.17-2.23 (m, 2H), δ2.30-2.34 (m, 2H), δ2.47-2.49 (m, 2H), δ2.50-2.54 (m, 4H), δ2.56-2.60 (m, 1H), δ2.85-2.93 (m, 5H), δ3.21-3.26 (m, 6H), δ3.43 (s, 3H), δ4.17-4.34 (m, 3H), δ5.05-5.07 (m, 1H), δ7.00-7.06 (m, 3H), δ7.18-7.20 (m, 1H), δ7.31-7.35 (m, 2H), δ7.42-7.44 (m, 1H), δ7.46-7.52 (m, 1H), δ7.65-7.73 (m, 3H), δ8.32 (s, 1H), δ10.94 (s, 1H).

### Example 135. Compound 137

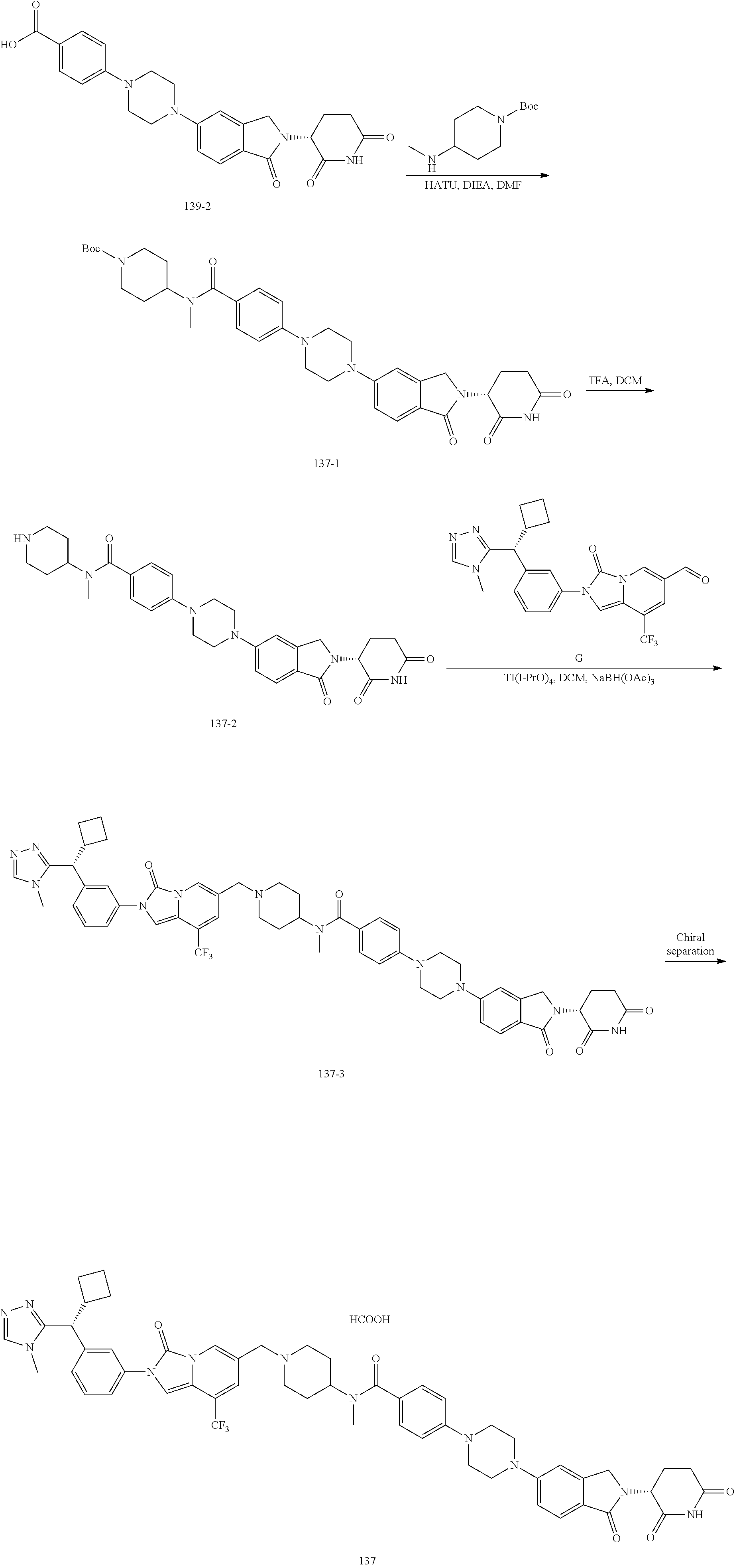

### Synthesis of Compound 137-1.

To a stirred solution of Compound 139-2 (2.2 g, 4.905 mmol, 1 equiv) and HATU (3.36 g, 8.829 mmol, 1.8 equiv) in DMF (25 mL) was added DIEA (2.54 g, 19.620 mmol, 4 equiv) at room temperature. The resulting mixture was stirred for 2 h at room temperature. To the above mixture was added tert-butyl 4-(methylamino)piperidine-1-carboxylate (1.05 g, 4.905 mmol, 1 equiv) at room temperature. The resulting mixture was stirred overnight at room temperature. The resulting mixture was diluted with water (50 mL) at room temperature. The precipitated solids were collected by filtration and washed with EtOAc (3×20 mL). This resulted in Compound 137-1 (3 g, 94.85%) as a brown solid.

### Synthesis of Compound 137-2.

To a stirred solution of Compound 137-1 (2.98 g, 4.622 mmol, 1 equiv) and DCM (40 mL) was added TFA (8 mL) at room temperature. The resulting mixture was stirred for 1 h at room temperature. The resulting mixture was concentrated under vacuum. The residue was purified by trituration with EtOAc (50 mL). This resulted in Compound 137-2 (2.5 g, 99.31%) as off-white solid.

### Synthesis of Compound 137-3.

To a stirred solution of Compound 137-2 (1.5 g, 2.754 mmol, 1 equiv) and Intermediate G (1.76 g, 3.856 mmol, 1.4 equiv) in DCM (25 mL) was added Ti(Oi-Pr)₄ (1335.83 mg, 4.701 mmol, 3 equiv) at room temperature. The resulting mixture was stirred overnight at room temperature. To the above mixture was added NaBH(OAc)₃ (1.17 g, 5.508 mmol, 2 equiv) at room temperature. The resulting mixture was stirred for additional 6 h at room temperature. The reaction was quenched with 10% HOAc (aq.) (150 mL) at room temperature. The aqueous layer was extracted with EtOAc (50 mL). The aqueous layer was filtered, the filter cake was washed with water (10 mL). The filtrate was concentrated under reduced pressure. The residue was purified by reverse flash chromatography with the following conditions: column, C18 silica gel; mobile phase, MeCN in water (0.1% FA), 10% to 50% gradient in 35 min; detector, UV 254 nm. This resulted in Compound 137-3 (450 mg, 16.60%) as a yellow solid.

### Synthesis of Compound 137.

The Compound 137-3 (450 mg) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 90% B to 90% B in 19 min; Wave Length: 220/254 nm; RT1 (min): 7.21; RT2 (min): 14.44; the first peak is product) to afford the crude product. The crude product was purified by Prep-HPLC with the following conditions (Column: Sunfire prep C18 column, 30*150 mm, 5 μm; Mobile Phase A: water (0.1% FA), Mobile Phase B: ACN; Flow rate: 60 mL/min; Gradient: 20% B to 30% B in 9 min, Wave Length: 254; 220 nm; RT1 (min): 8.03) to afford Compound 137 (104.4 mg, 23.2%) as a yellow solid.

LC-MS-Compound 137: (ES, m/z): [M−HCOOH+H]⁺ 984

H-NMR-Compound 137: (400 MHz, DMSO-d6, δ ppm): 1.61-1.69 (m, 2H), 1.70-1.85 (m, 8H), 1.90-1.98 (m, 2H), 2.07-2.12 (m, 1H), 2.30-2.42 (m, 1H), 2.55-2.65 (m, 1H), 2.87 (s, 3H), 2.87-2.94 (m, 3H), 3.15-3.27 (m, 4H), 3.34-3.46 (m, 7H), 3.50-3.52 (m, 4H), 4.21-4.36 (m, 3H), 5.05-5.08 (d, 1H), 7.00-7.03 (m, 3H), 7.13-7.20 (m, 3H), 7.27-7.32 (m 3H), 7.41-7.45 (m, 1H), 7.55-7.57 (d, 1H), 7.6-7.72 (m, 3H), 8.14 (s, 1H), 8.32 (s, 1H), 10.95 (s, 1H).

### Example 136. Compound 138

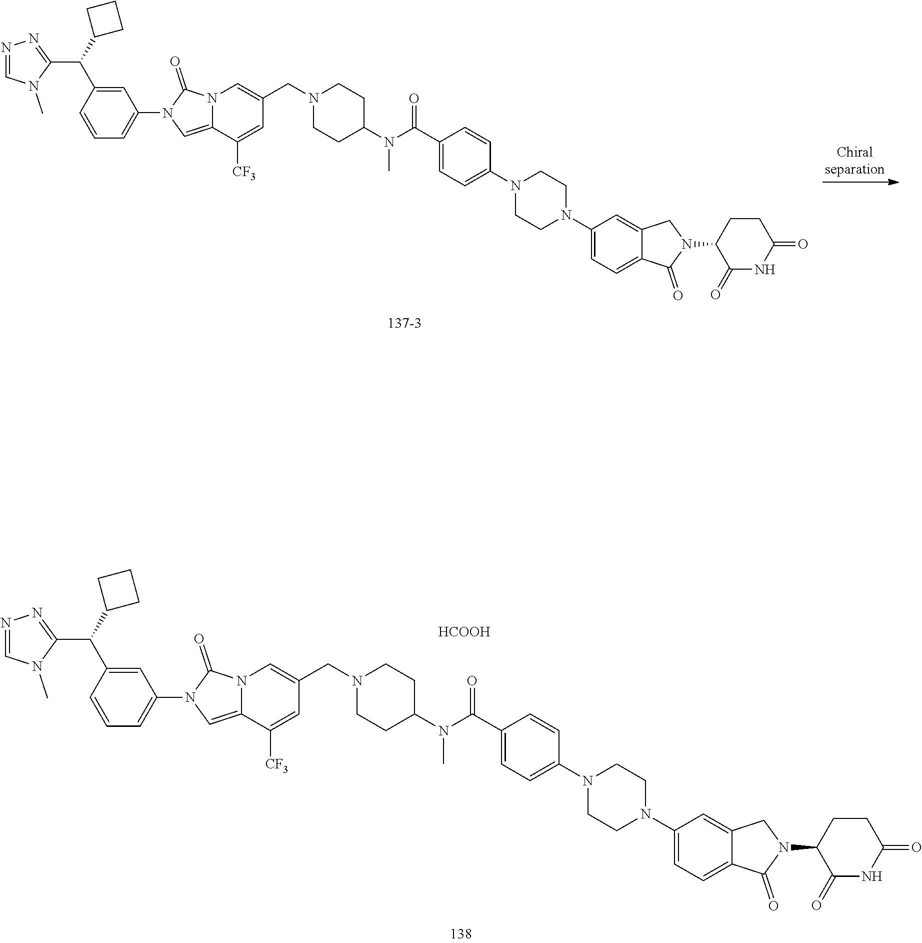 [SMILES: CN(C(=O)c1ccc(N2CCN(c3ccc4c(c3)CN([C@@H]3CCC(=O)NC3=O)C4=O)CC2)cc1)C1CCN(Cc2cc(C(F)(F)F)c3cn(-c4cccc([C@H](c5nncn5C)C5CCC5)c4)c(=O)n3c2)CC1 ; CN(C(=O)c1ccc(N2CCN(c3ccc4c(c3)CN([C@H]3CCC(=O)NC3=O)C4=O)CC2)cc1)C1CCN(Cc2cc(C(F)(F)F)c3cn(-c4cccc([C@H](c5nncn5C)C5CCC5)c4)c(=O)n3c2)CC1 ; "HCOOH"]

### Synthesis of Compound 138.

The Compound 137-3 (450 mg) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 90% B to 90% B in 19 min; Wave Length: 220/254 nm; RT1 (min): 7.21; RT2 (min): 14.44; the second peak is product) to afford the crude product. The crude product was purified by Prep-HPLC with the following conditions (Column: Sunfire prep C18 column, 30*150 mm, 5 μm; Mobile Phase A: water (0.1% FA), Mobile Phase B: ACN; Flow rate: 60 mL/min; Gradient: 20% B to 32% B in 8 min, Wave Length: 254; 220 nm; RT1 (min): 7.57) to afford Compound 137 (105.5 mg, 23.4%) as a yellow solid.

LC-MS-Compound 138: (ES, m/z): [M−HCOOH+H]⁺ 984

H-NMR-Compound 138: (400 MHz, DMSO-d₆, δ ppm): 1.61-1.85 (m, 10H), 1.90-1.98 (m, 2H), 2.07-2.12 (m, 1H), 2.30-2.42 (m, 1H), 2.55-2.65 (m, 1H), 2.87 (s, 3H), 2.87-2.94 (m, 3H), 3.15-3.27 (m, 4H), 3.34-3.46 (m, 7H), 3.50-3.52 (m, 4H), 4.21-4.27 (m, 2H), 4.33-4.35 (m, 2H), 5.05-5.08 (d, 1H), 7.00-7.03 (m, 3H), 7.13-7.20 (m, 3H), 7.27-7.31 (m 3H), 7.41-7.45 (m, 1H), 7.55-7.57 (d, 1H), 7.6-7.72 (m, 3H), 8.14 (s, 1H), 8.32 (s, 1H), 10.95 (s, 1H).

### Example 137. Compound 139

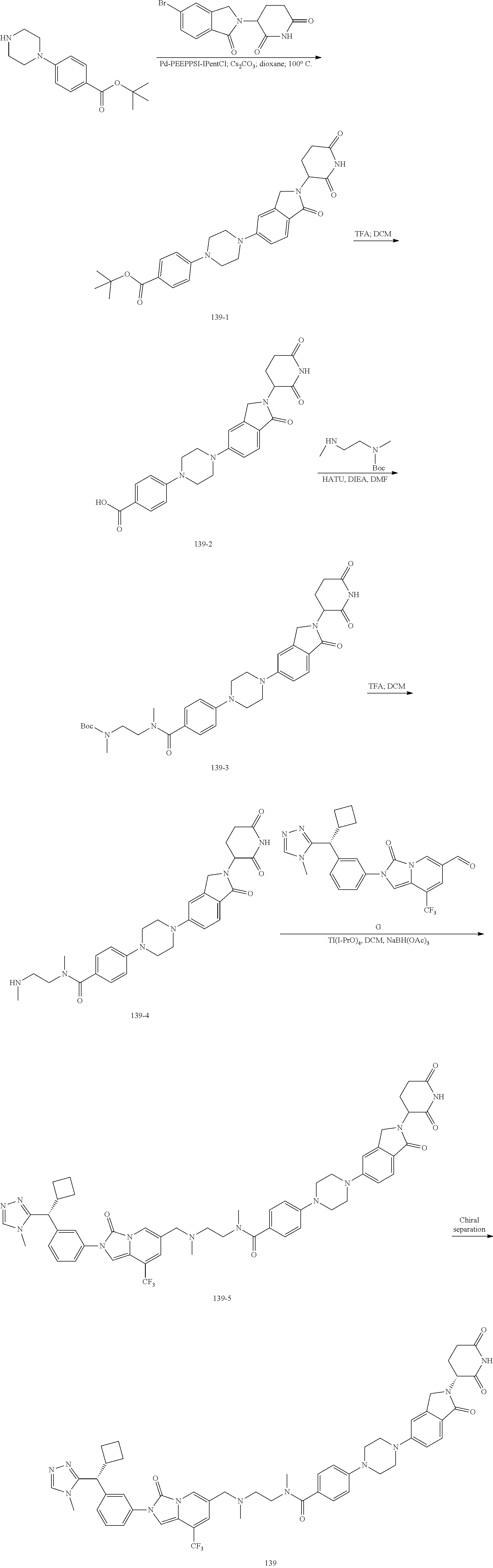

### Synthesis of Compound 139-1.

To a stirred solution of tert-butyl 4-(piperazin-1-yl)benzoate (20 g, 76.233 mmol, 1.00 equiv) and 3-(5-bromo-1-oxo-3H-isoindol-2-yl)piperidine-2,6-dione (36.95 g, 114.350 mmol, 1.5 equiv) in dioxane (500 mL) was added Cs₂CO₃ (74.51 g, 228.699 mmol, 3 equiv) and Pd-PEPPSI-IPcntCl 2-methylpyridine (o-picoline (4.49 g, 5.336 mmol, 0.07 equiv) at room temperature under nitrogen atmosphere. The resulting mixture was stirred overnight at 100° C. under nitrogen atmosphere. The mixture was allowed to cool down to room temperature. The mixture was acidified to pH 6 with HOAc. The resulting mixture was concentrated under vacuum. The residue was purified by silica gel column chromatography, eluted with DCM (0.1% HOAc)/MeOH (30:1) to afford Compound 139-1 (30 g, 70.19%) as an off-white solid.

### Synthesis of Compound 139-2.

To a stirred solution of Compound 139-1 (30 g, 59.455 mmol, 1 equiv) in DCM (300 mL) was added TFA (50 mL) at room temperature. The resulting mixture was stirred for 4 h at room temperature. The resulting mixture was concentrated under vacuum. The product was precipitated by the addition of MeCN. This resulted in Compound 139-2 (25 g, 90.95%) as an off-white solid.

### Synthesis of Compound 139-3.

To a stirred solution of Compound 139-2 (9 g, 20.068 mmol, 1 equiv) and tert-butyl N-methyl-N-[2-(methylamino)ethyl]carbamate (4.53 g, 24.061 mmol, 1.20 equiv) in DMF (100 mL) was added HATU (9.92 g, 26.088 mmol, 1.3 equiv) and DIEA (5.19 g, 40.136 mmol, 2 equiv) at room temperature. The resulting mixture was stirred overnight at room temperature. The residue was purified by reverse flash chromatography with the following conditions: column, C18 silica gel; mobile phase, MeCN in water (0.1% FA), 0% to 50% gradient in 30 min; detector, UV 254 nm. This resulted in Compound 139-3 (5 g, 38.26%) as an off-white solid.

### Synthesis of Compound 139-4.

A solution of Compound 139-3 (5 g, 8.081 mmol, 1 equiv) and TFA (5 mL) in DCM (20 mL) was stirred for 3 h at room temperature. The resulting mixture was concentrated under vacuum. This resulted in 4-Compound 139-4 (4 g, 85.90%) as an off-white solid.

### Synthesis of Compound 139-5.

To a stirred solution of Compound 139-4 (2.5 g, 4.821 mmol, 1 equiv) and Intermediate G (1.76 g, 3.857 mmol, 0.8 equiv) in DCM (30 mL) was added Ti(Oi-Pr)₄ (4.11 g, 14.463 mmol, 3 equiv) at room temperature. The resulting mixture was stirred overnight at room temperature. To the above mixture was added NaBH(OAc)₃ (4.09 g, 19.284 mmol, 4 equiv) at room temperature. The resulting mixture was stirred for additional 6 h at room temperature. The reaction was quenched with 10% HOAc (aq.) (150 mL) at room temperature. The aqueous layer was extracted with EtOAc (50 mL). The aqueous layer was filtered, the filter cake was washed with water 10% HOAc (aq.) (10 mL). The filtrate was concentrated under reduced pressure. The residue was purified by reverse flash chromatography with the following conditions (column, C18 silica gel; mobile phase, MeCN in Water (0.1% FA), 10% to 50% gradient in 20 min; detector, UV 254 nm) to afford the crude product. The crude product (700 mg) was purified by Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 22% B to 33% B in 7 min, Wave Length: 254; 220 nm; RT1 (min): 6.52) to afford Compound 139-5 (0.350 mg, 7.35%) as a yellow solid.

### Synthesis of Compound 139.

The Compound 139-5 (350 mg) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 90% B to 90% B in 8 min; Wave Length: 220/254 min; RT1 (min): 3.7; RT2 (min): 5.70; the first peak is product) to afford Compound 139 (121.9 mg, 34.03%) as a yellow solid.

LC-MS-Compound 139: (ES, m/z): [M+H]⁺ 958

H-NMR-Compound 139: (400 MHz, DMSO-d₆, δ ppm): 1.69-1.85 (m, 5H), 1.95-2.31 (m, 5H), 2.31-2.40 (m, 1H), 2.50-2.67 (m, 2H), 2.83-3.05 (m, 4H), 3.20-3.25 (m, 2H), 3.25-3.32 (m, 5H), 3.35-3.42 (m, 10H), 4.20-4.37 (m, 3H), 5.04-5.08 (d, 1H), 6.95-7.01 (m, 3H), 7.10-7.12 (m, 2H), 7.12-7.18 (m, 1H), 7.26-7.28 (m, 2H), 7.33 (s, 1H), 7.42-7.46 (m, 1H), 7.54-7.56 (d, 1H), 7.68-7.75 (m, 3H), 8.32 (s, 1H), 10.95 (s, 1H).

### Example 138. Compound 140

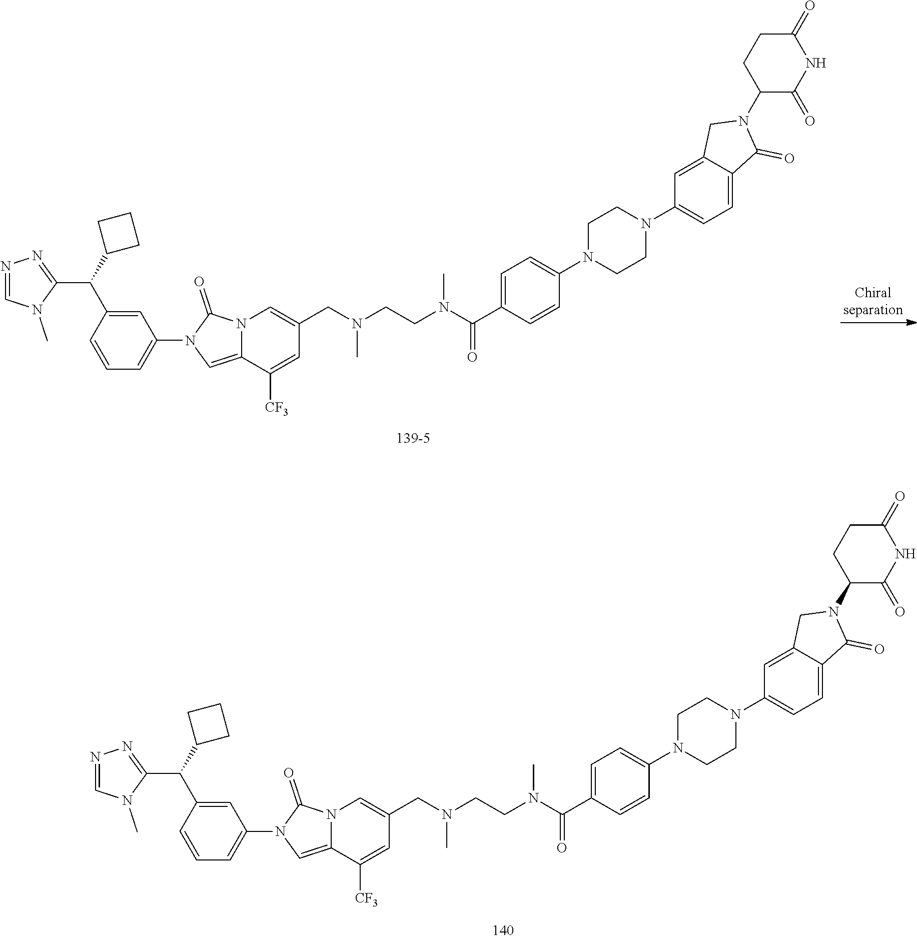 [SMILES: CN(CCN(C)C(=O)c1ccc(N2CCN(c3ccc4c(c3)CN(C3CCC(=O)NC3=O)C4=O)CC2)cc1)Cc1cc(C(F)(F)F)c2cn(-c3cccc([C@H](c4nncn4C)C4CCC4)c3)c(=O)n2c1 ; CN(CCN(C)C(=O)c1ccc(N2CCN(c3ccc4c(c3)CN([C@H]3CCC(=O)NC3=O)C4=O)CC2)cc1)Cc1cc(C(F)(F)F)c2cn(-c3cccc([C@H](c4nncn4C)C4CCC4)c3)c(=O)n2c1]

### Synthesis of Compound 140.

The Compound 139-5 (350 mg) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 90% B to 90% B in 8 min; Wave Length: 220/254 nm; RT1 (min): 3.7; RT2 (min): 5.70; the second peak is product) to afford Compound 140 (129.5 mg, 36.59%) as a yellow solid.

LC-MS-Compound 140: (ES, m/z): [M+H]⁺ 958

H-NMR-Compound 140: (400 MHz, DMSO-d₆, δ ppm): 1.69-1.85 (m, 5H), 1.95-2.31 (m, 5H), 2.31-2.40 (m, 1H), 2.50-2.67 (m, 2H), 2.83-3.05 (m, 4H), 3.20-3.25 (m, 1H), 3.25-3.32 (m, 11H), 3.35-3.42 (m, 4H), 4.20-4.37 (m, 3H), 5.04-5.08 (d, 1H), 6.95-7.01 (m, 3H), 7.10-7.12 (m, 2H), 7.12-7.18 (m, 1H), 7.26-7.28 (m, 2H), 7.33 (s, 1H), 7.42-7.46 (m, 1H), 7.54-7.56 (d, 1H), 7.68-7.75 (m, 3H), 8.32 (s, 1H), 10.95 (s, 1H).

### Example 139. Compound 141

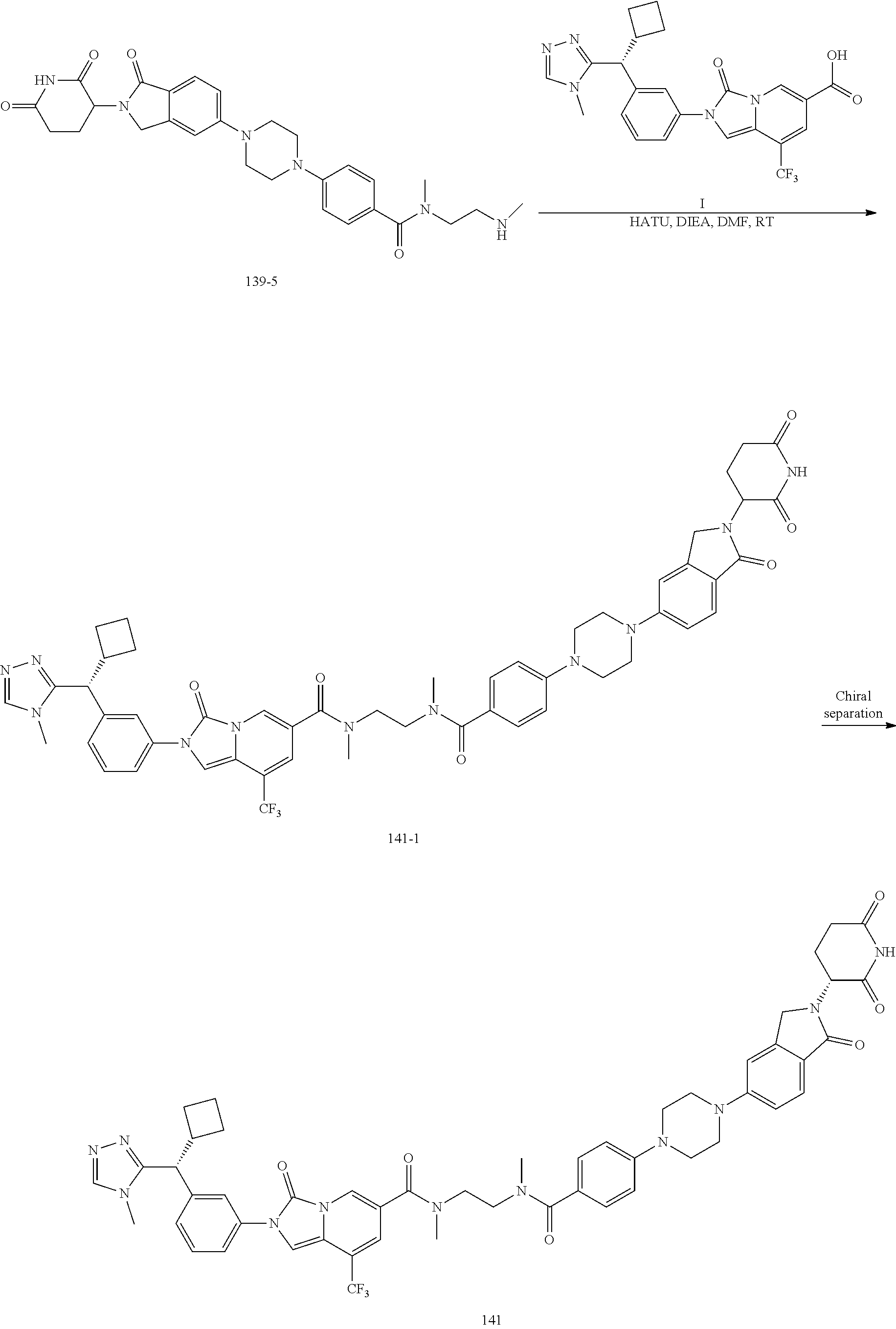

### Synthesis of Compound 141-1.

To a stirred solution of Compound 139-5 (1.5 g, 2.892 mmol, 1 equiv) and Intermediate I (1.36 g, 2.892 mmol, 1 equiv) in DMF (20 mL) was added HATU (1.32 g, 3.470 mmol, 1.2 equiv) and DIEA (0.75 g, 5.784 mmol, 2 equiv) at room temperature. The resulting mixture was stirred for 4 h at room temperature. The residue was purified by reverse flash chromatography with the following conditions (column, C18 silica gel; mobile phase, MeCN in Water (0.1% FA), 0% to 50% gradient in 20 min; detector, UV 254 nm) to afford the crude product. The crude product (500 mg) was purified by Prep-HPLC with the following conditions (Column: Xselect CSH (18 OBD Column 30*150 mm 5 μm, n; Mobile Phase A: Water (0.1% FA), Mobile Phase B: ACN; Flow rate: 60 mL/min; Gradient: 30% B to 43% B in 8 min, Wave Length: 254; 220 nm; RT1 (min): 7.63) to afford Compound 141-1 (200 mg, 6.90%) as a yellow solid.

### Synthesis of Compound 141.

The Compound 141-1 (200 mg) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 90% B to 90% B in 9.5 min; Wave Length: 220/254 nm; RT1 (min): 4.52; RT2 (min): 6.92; the first peak is product) to afford Compound 141 (80.9 mg, 40.21%) as a yellow solid.

LC-MS-Compound 141: (ES, m/z): [M+H]⁺ 972

H-NMR-Compound 141: (400 MHz, DMSO-d₆, δ ppm): 1.69-1.85 (m, 5H), 1.91-2.03 (m, 1H), 2.07-2.15 (m, 1H), 2.31-2.42 (m, 1H), 2.50-2.67 (m, 1H), 2.71-3.29 (m, 8H), 3.20-3.25 (m, 4H), 3.32-3.49 (m, 7H), 3.59-3.82 (m, 4H), 4.20-4.37 (m, 3H), 5.04-5.08 (d, 1H), 6.95-7.01 (m, 3H), 7.10-7.13 (m, 2H), 7.20-7.22 (d, 1H), 7.27-7.29 (m, 2H), 7.42-7.47 (m, 2H), 7.54-7.56 (d, 1H), 7.67-7.69 (m, 1H), 7.76-7.81 (m, 2H), 8.32 (s, 1H), 10.95 (s, 1H).

### Example 140. Compound 142

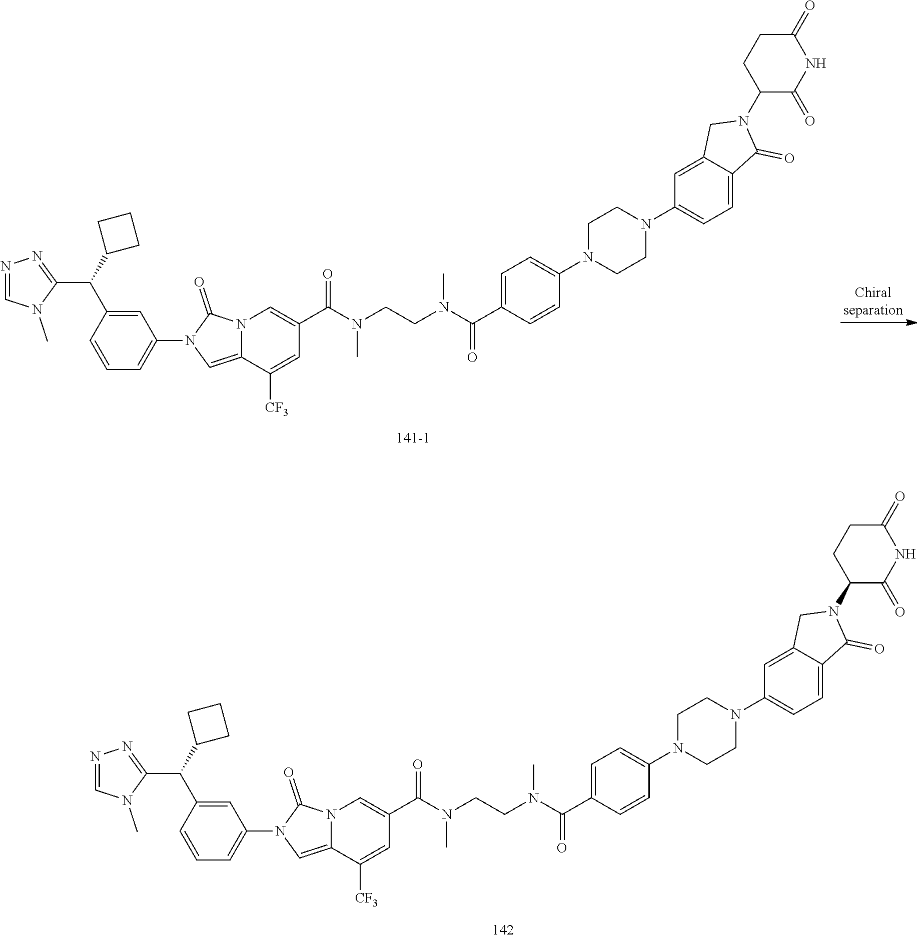 [SMILES: CN(CCN(C)C(=O)c1cc(C(F)(F)F)c2cn(-c3cccc([C@H](c4nncn4C)C4CCC4)c3)c(=O)n2c1)C(=O)c1ccc(N2CCN(c3ccc4c(c3)CN(C3CCC(=O)NC3=O)C4=O)CC2)cc1 ; CN(CCN(C)C(=O)c1cc(C(F)(F)F)c2cn(-c3cccc([C@H](c4nncn4C)C4CCC4)c3)c(=O)n2c1)C(=O)c1ccc(N2CCN(c3ccc4c(c3)CN([C@H]3CCC(=O)NC3=O)C4=O)CC2)cc1]

### Synthesis of Compound 142.

The Compound 141-1 (200 mg) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 90% B to 90% B in 9.5 min; Wave Length: 220/254 nm; RT1 (min): 4.52; RT2 (min): 6.92; the second peak is product) to afford Compound 142 (80.1 mg, 39.81%) as a yellow solid.

LC-MS-Compound 142: (ES, m/z): [M+H]⁺ 972

H-NMR-Compound 142: (400 MHz, DMSO-d₆, δ ppm): 1.69-1.85 (m, 5H), 1.91-2.03 (m, 1H), 2.07-2.15 (m, 1H), 2.31-2.42 (m, 1H), 2.50-2.67 (m, 1H), 2.71-3.29 (m, 8H), 3.20-3.25 (m, 4H), 3.32-3.49 (m, 7H), 3.59-3.82 (m, 4H), 4.20-4.37 (m, 3H), 5.04-5.08 (d, 1H), 6.95-7.01 (m, 3H), 7.10-7.13 (m, 2H), 7.20-7.22 (d, 1H), 7.27-7.29 (m, 2H), 7.42-7.47 (m, 2H), 7.54-7.56 (d, 1H), 7.67-7.69 (m, 1H), 7.76-7.81 (m, 2H), 8.32 (s, 1H), 10.95 (s, 1H).

### Example 141. Compound 143

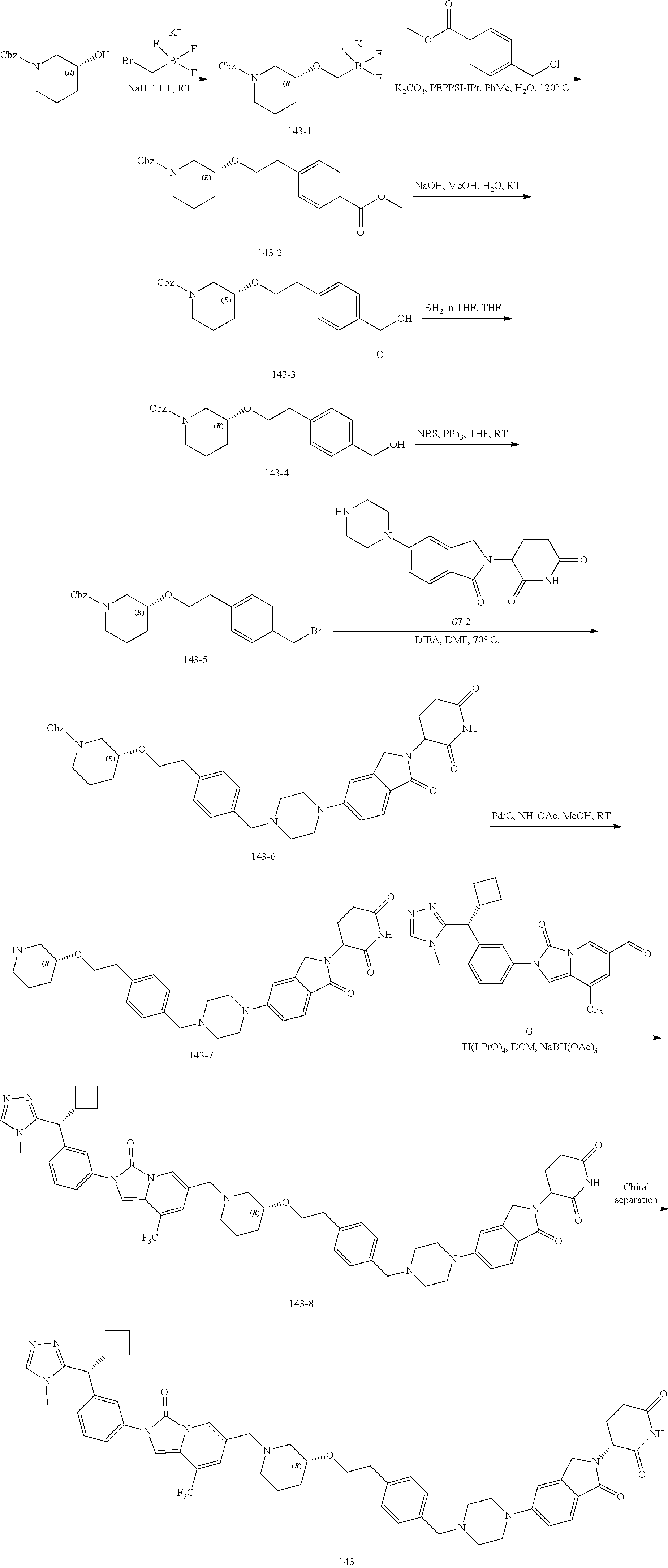

### Synthesis of Compound 143-1.

To a stirred mixture of NaH (6.8 g, 170.008 mmol, 2 equiv, 60%) in THF (200 mL) was added benzyl (3R)-3-hydroxypiperidine-1-carboxylate (20 g, 85.004 mmol, 1 equiv) at 0° C. under nitrogen atmosphere. The resulting mixture was stirred for 1 h at 0° C. under nitrogen atmosphere. To the above mixture was added potassium (bromomethyl)trifluoroboranuide (17.07 g, 85.004 mmol, 1 equiv) at 0° C. The resulting mixture was stirred overnight at room temperature. The resulting mixture was concentrated under vacuum. The residue was dissolved in acetone (200 mL). The resulting mixture was stirred for 2 h at 60° C. The resulting mixture was filtered, the filter cake was washed with acetone (2×50 mL). The filtrate was concentrated under reduced pressure. This resulted in Compound 143-1 (20 g, 74.43%) as a yellow oil.

### Synthesis of Compound 143-2.

To a stirred mixture of Compound 143-1 (20 g, 63.269 mmol, 1 equiv) and methyl 4-(chloromethyl)benzoate (17.52 g, 94.903 mmol, 1.5 equiv) in Toluene (180 mL)/H₂O (20 mL) was added PEPPSI-IPr (2.66 g, 3.163 mmol, 0.05 equiv) and K₂CO₃ (26.23 g, 189.807 mmol, 3 equiv) at room temperature. The resulting mixture was stirred overnight at 120° C. The mixture was allowed to cool down to room temperature. The residue was diluted with water (400 mL). The aqueous layer was extracted with EtOAc (3×200 mL). The resulting mixture was concentrated under vacuum. The residue was purified by silica gel column chromatography, eluted with PE/EA (5:1) to afford benzyl Compound 143-2 (2.7 g, 10.74%) as a yellow green oil.

### Synthesis of Compound 143-3.

To a stirred mixture of Compound 143-2 (2.7 g, 6.793 mmol, 1 equiv) in MeOH (15 mL) was added NaOH (1M) (13.5 mL) at room temperature. The resulting mixture was stirred for 2 h at room temperature. The resulting mixture was concentrated under vacuum. The mixture was acidified to pH 2 with 4M HCl (aq.). The precipitated solids were collected by filtration and washed with water (10 mL). This resulted in Compound 143-3 (2.1 g, 80.62%) as a yellow solid.

### Synthesis of Compound 143-4.

To a stirred mixture of Compound 143-3 (2.1 g, 5.477 mmol, 1 equiv) and THF (20 mL) was added BH₃-THF (11 mL, 10.954 mmol, 2 equiv) dropwise at 0° C. The resulting mixture was stirred for 2 h at 0° C. The reaction was quenched with NH₄Cl(aq.) at 0° C. The aqueous layer was extracted with EtOAc (3×100 mL). The resulting mixture was concentrated under vacuum. The residue was purified by silica gel column chromatography, eluted with PE/EA (1:1) to afford Compound 143-4 (2 g, 98.84%) as a yellow oil.

### Synthesis of Compound 143-5.

To a stirred mixture of Compound 143-4 (3.68 g, 9.960 mmol, 1 equiv) and NBS (2.66 g, 14.940 mmol, 1.5 equiv) in THF (40 mL) was added PPh₃ (3.92 g, 14.940 mmol, 1.5 equiv) at room temperature. The resulting mixture was stirred for 2 h at room temperature. The reaction was quenched with water at room temperature. The aqueous layer was extracted with EtOAc (3×100 mL). The resulting mixture was concentrated under vacuum. The residue was purified by silica gel column chromatography, eluted with PE/EA (5:1) to afford Compound 143-5 (2.7 g, 62.70%) as a brown solid.

### Synthesis of Compound 143-6.

To a stirred mixture of Compound 143-5 (2.7 g, 6.245 mmol, 1 equiv) and Compound 67-2 (2.26 g, 6.870 mmol, 1.1 equiv) in DMF (30 mL) was added DIEA (2.42 g, 18.735 mmol, 3 equiv) at room temperature. The resulting mixture was stirred for 4 h at 70° C. The mixture was allowed to cool down to room temperature. The residue was purified by reversed-phase flash chromatography with the following conditions: column, C18 silica gel; mobile phase, MeCN in water (0.1% FA), 10% to 50% gradient in 40 min; detector, UV 220 nm. This resulted in Compound 143-6 (1.7 g, 40.04%) as a yellow solid.

### Synthesis of Compound 143-7.

To a solution of Compound 143-6 (1.68 g, 2.471 mmol, 1 equiv) in MeOH (200 mL) was added Pd/C (10%, 0.16 g) and NH₄OAc (3.81 g, 49.42 mmol, 20 equiv) under nitrogen atmosphere in a 500 mL round-bottom flask. The mixture was hydrogenated at room temperature for 15 min under hydrogen atmosphere using a hydrogen balloon. The resulting mixture was filtered, the filter cake was washed with MeOH (3×30 mL). The filtrate was concentrated under reduced pressure. The residue was purified by reversed-phase flash chromatography with the following conditions: column, C18 silica gel; mobile phase, MeCN in water (0.1% FA), 10% to 40% gradient in 20 min; detector, UV 254 nm. This resulted in Compound 143-7 (1.2 g, 88.99%) as a white solid.

### Synthesis of Compound 143-8.

To a stirred solution of Compound 143-7 (1.2 g, 2.199 mmol, 1 equiv) and Intermediate G (1.30 g, 2.859 mmol, 1.3 equiv) in DCM (20 mL) was added Ti(Oi-Pr)₄ (1.25 g, 4.398 mmol, 2 equiv) at room temperature. The resulting mixture was stirred overnight at room temperature. To the above mixture was added NaBH(OAc)₃ (0.93 g, 4.398 mmol, 2 equiv) at room temperature. The resulting mixture was stirred for additional 4 h at room temperature. The reaction was quenched with 10% HOAc (aq.) (200 mL) at room temperature. The resulting mixture was filtered, the Filter cake was washed with 10% HOAc (aq.) (2×10 mL). The aqueous layer was extracted with EtOAc (100 mL). The aqueous layer was concentrated under vacuum. The residue was purified by reversed-phase flash chromatography with the following conditions: column, C18 silica gel; mobile phase, MeCN in water (0.1% FA), 10% to 100% gradient in 40 min; detector, UV 254 nm. This resulted in Compound 143-8 (200 mg, 9.23%) as a yellow solid.

### Synthesis of Compound 143.

The Compound 143-8 (200 mg) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 60% B to 60% B in 16 min; Wave Length: 220/254 nm; RT1 (min): 6.72; RT2 (min): 11.93; the first peak is product) to afford the crude product. The crude product was purified by reversed-phase flash chromatography with the following conditions: column, C18 silica gel; mobile phase, MeCN in water (0.1% FA), 10% to 35% gradient in 20 min; detector, UV 254 nm. This resulted in Compound 143 (44.1 mg, 21.83%) as a yellow solid.

LC-MS-Compound 143: (ES, m/z): [M+H]⁺ 985

H-NMR-Compound 143: (400 MHz, CD₃OD, δ ppm): 1.33-1.36 (m, 1H), 1.43-1.46 (m, 1H), 1.73-1.75 (m, 2H), 1.87-1.88 (m, 5H), 2.10-2.20 (m, 2H), 2.20-2.32 (m, 2H), 2.35-2.55 (m, 1H), 2.71-2.79 (m, 5H), 2.80-2.94 (m, 5H), 3.25-3.31 (m, 21H), 3.32-3.35 (m, 4H), 3.42-3.45 (m, 2H), 3.51 (s, 3H), 3.57 (s, 2H), 3.72-3.74 (m, 2H), 4.24-4.27 (d, 1H), 4.35-4.37 (m, 2H), 5.05-5.09 (m, 1H), 7.00-7.37 (m, 9H), 7.43-7.47 (m, 1H), 7.58-7.65 (m, 4H), 8.33 (s, 1H).

### Example 142. Compound 144

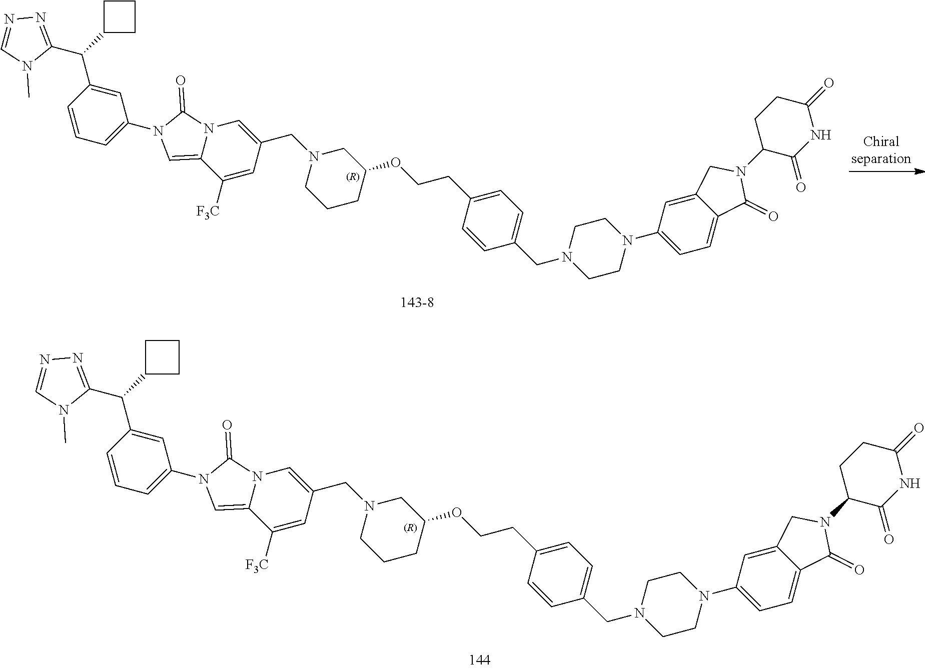

### Synthesis of Compound 144.

The Compound 143-8 (200 mg) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 60% B to 60% B in 16 min; Wave Length: 220/254 nm; RT1 (min): 6.72; RT2 (min): 11.93; the second peak is product) to afford the crude product. The crude product was purified by reversed-phase flash chromatography with the following conditions: column, C18 silica gel; mobile phase, MeCN in water (0.1% FA), 10% to 35% gradient in 20 min; detector, UV 254 nm. This resulted in Compound 144 (120.8 mg, 40.27%) as a yellow solid.

LC-MS-Compound 144: (ES, m/z): [M+H]⁺ 985

H-NMR-Compound 144: (400 MHz, CD₃OD, δ ppm): 1.33-1.36 (m, 1H), 1.43-1.46 (m, 1H), 1.73-1.75 (m, 2H), 1.87-1.88 (m, 5H), 2.10-2.32 (m, 2H), 2.35-2.55 (m, 1H), 2.71-2.79 (m, 5H), 2.80-2.94 (m, 5H), 3.25-3.31 (m, 2H), 3.32-3.35 (m, 4H), 3.42-3.45 (m, 2H), 3.51 (s, 3H), 3.57 (s, 2H), 3.72-3.74 (m, 2H), 4.24-4.27 (d, 1H), 4.35-4.37 (m, 2H), 5.05-5.09 (d, 1H), 7.00-7.37 (m, 9H), 7.43-7.47 (m, 1H), 7.58-7.65 (m, 4H), 8.33 (s, 1H).

### Example 143. Compound 145

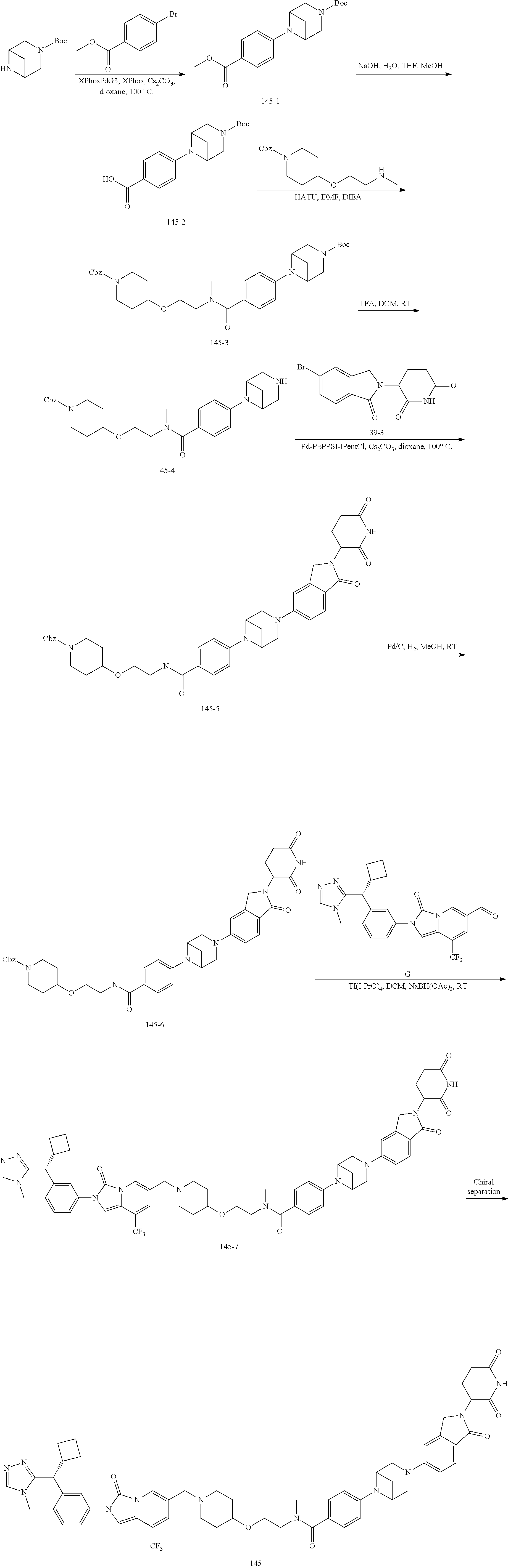

### Synthesis of Compound 145-1.

To a stirred solution of tert-butyl 3,6-diazabicyclo [3.1.1] heptane-3-carboxylate (9 g, 45.394 mmol, 1 equiv) and methyl 4-bromobenzoate (11.71 g, 54.473 mmol, 1.2 equiv) in dioxane (100 mL) was added Cs₂CO₃ (44.37 g, 136.182 mmol, 3 equiv), XPhos (2.16 g, 4.539 mmol, 0.1 equiv) and XPhos Pd G₃ (3.84 g, 9.079 mmol, 0.1 equiv) at room temperature under nitrogen atmosphere. The resulting mixture was stirred for overnight at 100° C. under nitrogen atmosphere. The mixture was acidified to pH 5 with AcOH. The resulting mixture was concentrated under reduced pressure. The residue was purified by silica gel column chromatography, eluted with PE/EA (10:1) to afford Compound 145-1 (10.25 g, 61.14%) as a white solid.

### Synthesis of Compound 145-2.

A solution of Compound 145-1 (10.25 g, 30.836 mmol, 1 equiv) and NaOH (12.33 g, 308.360 mmol, 10 equiv) in 1-20 (100 mL) and THF (100 mL) was stirred for 5 h at room temperature. The mixture was acidified to pH 4 with 1 M HCl(aq.). The precipitated solids were collected by filtration and washed with water (3×50 mL). This resulted in Compound 145-2 (9.4 g, 86.17%) as a white solid.

### Synthesis of Compound 145-3.

To a stirred solution of Compound 145-2 (8 g, 25.128 mmol, 1 equiv) and HATU (23.89 g, 62.820 mmol, 2.5 equiv) in DMF (80 mL) was added DIEA (8.12 g, 62.820 mmol, 2.5 equiv) at room temperature. The resulting mixture was stirred for 1 h at room temperature. To the above mixture was added benzyl 4-[2-(methylamino) ethoxy] piperidine-1-carboxylate (8.82 g, 30.154 mmol, 1.2 equiv) at room temperature. The resulting mixture was stirred for additional overnight at room temperature. The reaction was quenched by the addition of Water (250 mL) at room temperature. The aqueous layer was extracted with EtOAc (3×200 mL). The residue was purified by silica gel column chromatography, eluted with DCM/MeOH (80:1) to afford Compound 145-3 (8 g, 48.34%) as an orange solid.

### Synthesis of Compound 145-4.

To a stirred solution of Compound 145-3 (8 g, 13.497 mmol, 1 equiv) and TFA (10 mL, 134.631 mmol) in DCM (80 mL, 941.956 mmol, 69.79 equiv) at room temperature. The resulting mixture was stirred for 2 h at room temperature. The resulting mixture was concentrated under reduced pressure. The residue was purified by reversed-phase flash chromatography with the following conditions: column, C18 silica gel; mobile phase, MeCN in Water (0.1% FA), 30% to 50% gradient in 20 min; detector, UV 254 nm. This resulted in Compound 145-4 (1.2 g, 16.24%) as a brown solid.

### Synthesis of Compound 145-5.

To a stirred solution of Compound 145-4 (1.2 g, 2.436 mmol, 1 equiv) and Compound 39-3 (1.18 g, 3.654 mmol, 1.5 equiv) in dioxane (20 mL) was added Cs₂CO₃ (2.38 g, 7.308 mmol, 3 equiv) and {1,3-bis[2,6-bis(pentan-3-yl) phenyl]-4,5-dichloro-2,3-dihydro-1H-imidazol-2-yl} dichloro(2-methyl-1lambda4-pyridin-1-yl) palladium (0.10 g, 0.122 mmol, 0.05 equiv) at room temperature. The resulting mixture was stirred for overnight at 100° C. under nitrogen atmosphere. The mixture was acidified to pH 5 with AcOH. The resulting mixture was concentrated under reduced pressure. The residue was purified by silica gel column chromatography, eluted with DCM/MeOH (20:1) to afford Compound 145-5 (1 g, 50.28%) as a brown solid.

### Synthesis of Compound 145-6.

To a solution of Compound 145-5 (1 g, 1.361 mmol, 1 equiv) in 10 mL MeOH was added Pd/C (10%, 0.2 g) under nitrogen atmosphere. The mixture was hydrogenated at room temperature for overnight under hydrogen atmosphere using a hydrogen balloon, filtered through a Celite pad and concentrated under reduced pressure.to afford Compound 145-6 (400 mg, 44.04%) as a brown solid.

### Synthesis of Compound 145-7.

To a stirred solution of Compound 145-6 (1.4 g, 2.331 mmol, 1 equiv) and Intermediate G (1.06 g, 2.331 mmol, 1 equiv) in DCM (14 mL) was added tetrakis(propan-2-yloxy) titanium (1.99 g, 6.993 mmol, 3 equiv) at room temperature. The resulting mixture was stirred for overnight at room temperature. To the above mixture was added NaBH(OAc)₃ (0.99 g, 4.662 mmol, 2 equiv) at room temperature. The resulting mixture was stirred for additional 3 h at room temperature. The mixture was acidified to pH 5 with water (10% AcOH). The resulting mixture was concentrated under reduced pressure. The residue was purified by reversed-phase flash chromatography with the following conditions: column, C18 silica gel; mobile phase, MeCN in Water (0.1% FA), 30% to 50% gradient in 20 min; detector, UV 254 nm. This resulted in Compound 145-7 (350 mg, 13.72%) as a yellow solid.

### Synthesis of Compound 145.

The Compound 145-7 (360 mg, 0.329 mmol, 1 equiv, 95%) was purified by Chiral Separation with the following conditions (Column: CHIRAL ART Cellulose-SB, 2*25 cm, 5 μm; Mobile Phase A: MTBE (0.1% TEA)—HPLC, Mobile Phase B: DCM:ACN=1:1—HPLC; Flow rate: 20 mL/min; Gradient: 90% B to 90% B in 14 min; Wave Length: 220/254 nm; RT1 (min): 17.21; RT2 (min): 21.46, the first peak was product) to afford crude product (160 mg). The crude product (160 mg) was purified by reversed-phase flash chromatography with the following conditions: column, 40 g C18 silica gel; mobile phase, MeCN in Water (0.1% FA), 30% to 50% gradient in 12 min; detector, UV 254 nm to afford Compound 145 (108.6 mg, 31.12%) as a yellow solid.

LC-MS-Compound 145: (ES, m/z): [M+H]⁺ 1040

H-NMR-Compound 145: (400 MHz, DMSO-d6, δ ppm): 1.31-1.49 (s, 2H), 1.68-1.80 (m, 8H), 1.82-1.89 (m, 1H), 2.02-2.24 (m, 3H), 2.30-2.49 (m, 1H), 2.51-2.57 (m, 2H), 2.71-2.75 (m, 2H), 2.91-2.96 (m, 4H), 3.23-3.28 (m, 4H), 3.33-3.37 (m, 2H), 3.42-3.49 (m, 8H), 3.79-3.82 (m, 2H), 4.11-4.15 (m, 1H), 4.24-4.28 (m, 2H), 4.53-4.54 (m, 2H), 4.98-5.02 (m, 1H), 6.64-6.72 (m, 4H), 7.01 (s, 1H), 7.08-7.24 (m, 3H), 7.32 (s, 1H), 7.42-7.46 (m, 2H), 7.67-7.73 (m, 3H), 8.32 (s, 1H), 10.92 (s, 1H).

### Example 144. Compound 146

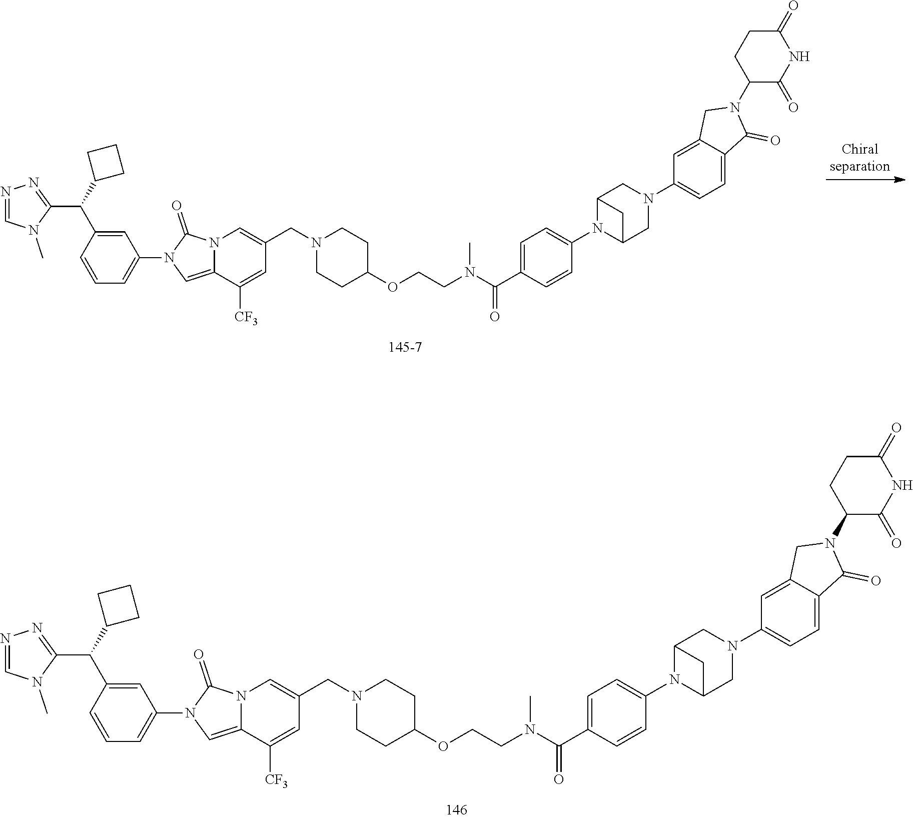 [SMILES: CN(CCOC1CCN(Cc2cc(C(F)(F)F)c3cn(-c4cccc([C@H](c5nncn5C)C5CCC5)c4)c(=O)n3c2)CC1)C(=O)c1ccc(N2C3CC2CN(c2ccc4c(c2)CN(C2CCC(=O)NC2=O)C4=O)C3)cc1 ; CN(CCOC1CCN(Cc2cc(C(F)(F)F)c3cn(-c4cccc([C@H](c5nncn5C)C5CCC5)c4)c(=O)n3c2)CC1)C(=O)c1ccc(N2C3CC2CN(c2ccc4c(c2)CN([C@H]2CCC(=O)NC2=O)C4=O)C3)cc1]

### Synthesis of Compound 146.

The Compound 145-7 (360 mg, 0.329 mmol, 1 equiv, 95%) was purified by Chiral Separation with the following conditions (Column: CHIRAL ART Cellulose-SB, 2*25 cm, 5 μm; Mobile Phase A: MTBE (0.1% TEA)—HPLC, Mobile Phase B: DCM:ACN=1:1—HPLC; Flow rate: 20 mL/min; Gradient: 90% B to 90% B in 14 min; Wave Length: 220/254 nm; RT1 (min): 17.21; RT2 (min): 21.46, the second peak was product) to afford crude product (150 mg). The crude product (150 mg) was purified by reversed-phase flash chromatography with the following conditions: column, 40 g C18 silica gel; mobile phase, MeCN in Water (0.1% FA), 30% to 45% gradient in 12 min; detector, UV 254 nm to afford Compound 146 (97.7 ng, 28.00%) as a yellow solid.

LC-MS-Compound 146: (ES, m/z): [M+H]⁺ 1040

H-NMR-Compound 146: (400 MHz, DMSO-d6, δ ppm): 1.29-1.58 (m, 2H), 1.69-1.98 (m, 10H), 2.09-2.18 (m, 2H), 2.32-2.49 (m, 1H), 2.51-2.57 (m, 1H), 2.72-2.73 (m, 1H), 2.87-2.91 (m, 4H), 3.09-3.21 (m, 3H), 3.22-3.24 (m, 2H), 3.33-3.49 (m, 9H), 3.79-3.82 (m, 2H), 4.12-4.16 (m, 1H), 4.24-4.28 (m, 2H), 4.53-4.54 (d, 2H), 5.01-5.02 (m, 1H), 6.64-6.66 (d, 2H), 6.70-6.72 (m, 2H), 7.01-7.07 (m, 1H), 7.19-7.21 (m, 3H), 7.24 (s, 1H), 7.42-7.46 (m, 2H), 7.68-7.73 (m, 3H), 8.32 (s, 1H), 10.92 (s, 1H).

### Example 145. Compound 147

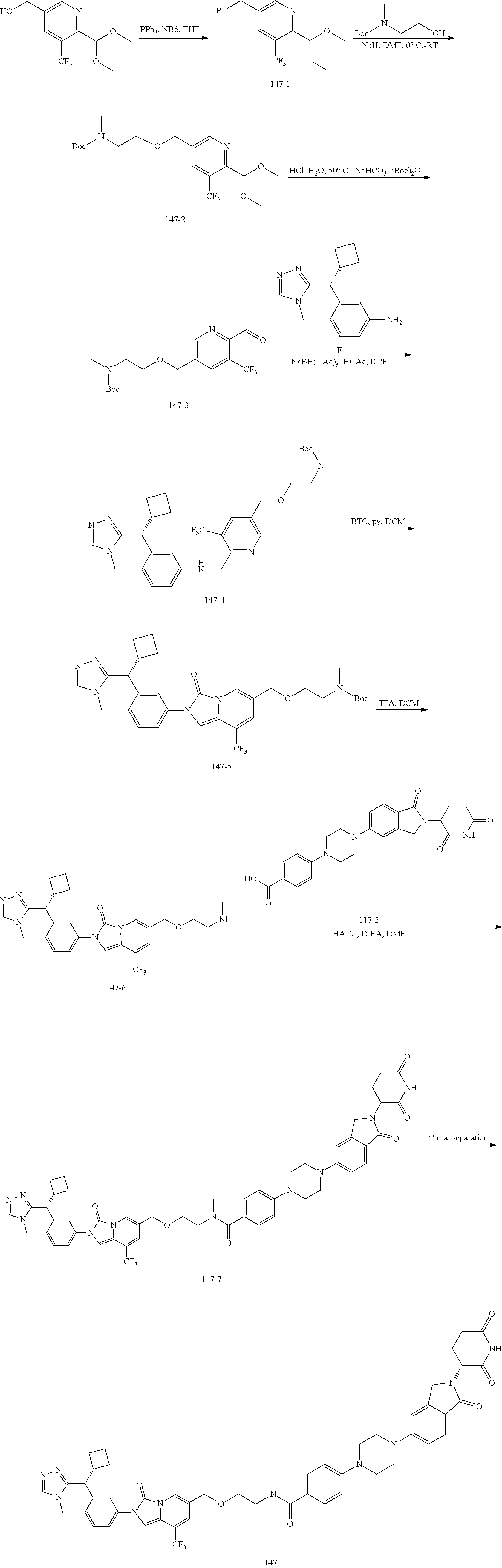

### Synthesis of Compound 147-1.

To a stirred mixture of [6-(dimethoxymethyl)-5-(trifluoromethyl)pyridin-3-yl]methanol (10 g, 39.808 mmol, 1 equiv) and NBS (10.63 g, 59.712 mmol, 1.5 equiv) in THF (200 mL) was added PPh₃ (15.66 g, 59.712 mmol, 1.5 equiv) at 0° C. The resulting mixture was stirred for 4 h at room temperature under nitrogen atmosphere. The resulting mixture was concentrated under reduced pressure. The residue was purified by silica gel column chromatography, eluted with PE/EA (5:1) to afford Compound 147-1 (9.5 g, 75.98%) as a yellow oil.

### Synthesis of Compound 147-2.

To a stirred solution of tert-butyl (2-hydroxyethyl)(methyl)carbamate (6.36 g, 36.294 mmol, 1.2 equiv) in DMF (100 mL) was added NaH (1.45 g, 36.294 mmol, 1.2 equiv, 60%) in portions at 0° C. under nitrogen atmosphere. The resulting mixture was stirred for 0.5 h at 0° C. under nitrogen atmosphere. To the above mixture was added Compound 147-1 (9.5 g, 30.245 mmol, 1 equiv) at 0° C. The resulting mixture was stirred for additional overnight at room temperature. The reaction was quenched by the addition of sat. NH₄Cl (aq.) (300 mL). The aqueous layer was extracted with EtOAc (2×150 mL). The residue was purified by silica gel column chromatography, eluted with CH₂Cl₂/MeOH (3:1) to afford Compound 147-2 (3.8 g, 30.76%) as a light yellow oil.

### Synthesis of Compound 147-3.

To a stirred solution of Compound 147-2 (3.78 g, 9.255 mmol, 1 equiv) in THF (40 mL) was added HCl (20 mL, 20.000 mmol, 2.16 equiv, 1M). The resulting mixture was stirred for overnight at 50° C. To the above mixture was added NaHCO₃ (3.89 g, 46.275 mmol, 5 equiv) and (Boc)₂O (4.04 g, 18.510 mmol, 2 equiv). The resulting mixture was stirred for additional 2 h at room temperature. The resulting mixture was diluted with water (40 mL). The resulting mixture was extracted with EtOAc (2×50 mL). The combined organic layers were concentrated under reduced pressure. The residue was purified by silica gel column chromatography, eluted with PE/EA (2:1) to afford Compound 147-3 (3 g, 89.46%) as a light yellow oil.

### Synthesis of Compound 147-4.

A solution of tert-butyl Compound 147-3 (1.5 g, 4.140 mmol, 1 equiv) in DCE (30 mL) was treated with Intermediate F (1.00 g, 4.140 mmol, 1 equiv) for overnight at room temperature under nitrogen atmosphere followed by the addition of NaBH(OAc)₃ (1.75 g, 8.280 mmol, 2 equiv) at room temperature. The resulting mixture was stirred for 5 h at mom temperature under nitrogen atmosphere. The reaction was quenched by the addition of sat. NaHCO₃(aq.) (50 mL). The resulting mixture was extracted with CH₂Cl₂/MeOOH=10/1 (2×60 mL). The combined organic layers were concentrated under reduced pressure. The residue was purified by silica gel column chromatography, eluted with CH₂Cl₂/MeOH (9:1) to afford Compound 147-4 (1.2 g, 49.24%) as a white solid.

### Synthesis of Compound 147-5.

To a stirred solution of Compound 147-4 (1.2 g, 2.038 mmol, 1 equiv) and pyridine (0.97 g, 12.228 mmol, 6 equiv) in DCM (36 mL) was added triphosgene (0.21 g, 0.713 mmol, 0.35 equiv) at 0° C. under nitrogen atmosphere. The resulting mixture was stirred for 0.5 h at 0° C. under nitrogen atmosphere. The reaction was quenched by the addition of sat. NaHCO₃(aq.) (100 mL). The resulting mixture was extracted with CH₂Cl₂/MeOH=10/1 (3×100 mL). The combined organic layers were concentrated under reduced pressure. The residue was purified by silica gel column chromatography, eluted with CH₂Cl₂/MeOH (7:1) to afford Compound 147-5 (1.1 g, 87.79%) as a yellow solid.

### Synthesis of Compound 147-6.

To a stirred solution of Compound 147-5 (1.1 g, 1.790 mmol, 1 equiv) in DCM (10 mL) was added TFA (2 mL). The resulting mixture was stirred for 3 h at room temperature. The resulting mixture was concentrated under reduced pressure. The residue was purified by Prep-TLC (CH₂Cl₂/MeOH 8:1) to afford Compound 147-6 (700 mg, 76.02%) as a yellow solid.

### Synthesis of Compound 147-7.

To a stirred solution of Compound 147-6 (680 mg, 1.322 mmol, 1 equiv) and Compound 117-2 (592.68 mg, 1.322 mmol, 1 equiv) in DMF (10 mL) was added DIEA (341.61 mg, 2.644 mmol, 2 equiv) and HATU (753.74 mg, 1.983 mmol, 1.5 equiv). The resulting mixture was stirred for overnight at room temperature. The reaction solution was purified by reverse flash chromatography with the following conditions: column, C18 silica gel; mobile phase, MeCN in Water (0.1% FA), 10% to 60% gradient in 10 min; detector, UV 254 nm. This resulted in Compound 147-7 (400 mg, 32.03%) as a yellow solid.

### Synthesis of Compound 147.

Compound 147-7 (400 mg) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 90% B to 90% B in 20 min; Wave Length: 220/254 nm; RT1 (min): 7.50; RT2 (min): 14.28; the first peak was product) to afford Compound 147 (140.7 mg, 35.17%) as a yellow solid.

LCMS-Compound 147: (ES, m/z): [M+H]⁺ 945

NMR-Compound 147: (400 MHz, DMSO, δ ppm): 61.69-1.80 (m, 5H), 1.90-1.99 (m, 1H), 2.02-2.11 (m, 1H), 2.33-2.40 (m, 1H), 2.50-2.64 (m, 1H), 2.82-3.01 (m, 4H), 3.15-3.24 (m, 1H), 3.35-3.48 (m, 11H), 3.57-3.62 (m, 4H), 4.20-4.37 (m, 5H), 5.03-5.08 (m, 1H), 6.97-7.03 (m, 3H), 7.10-7.21 (m, 2H), 7.29-7.35 (m, 4H), 7.42-7.46 (t, 1H), 7.54-7.56 (m, 1H), 7.70-7.72 (m, 1H), 7.76-7.80 (m, 2H), 8.31 (s, 1H), 10.96 (s, 1H).

### Example 146. Compound 148

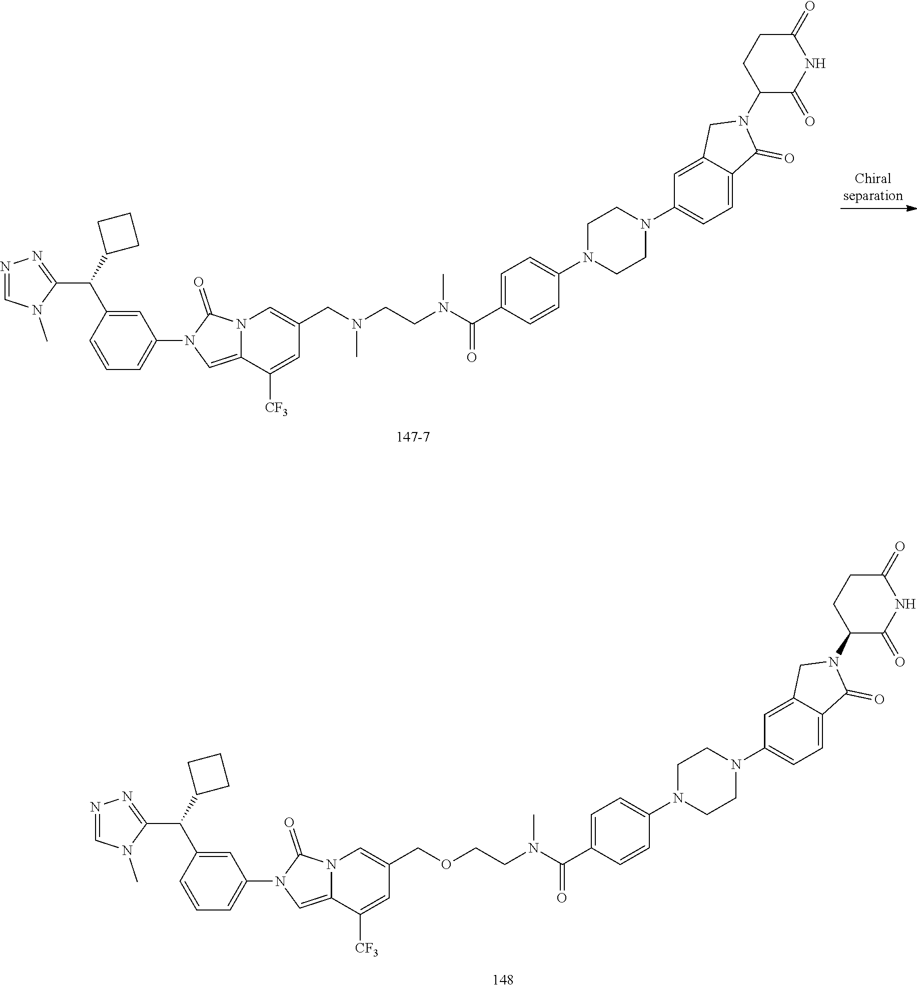 [SMILES: CN(CCN(C)C(=O)c1ccc(N2CCN(c3ccc4c(c3)CN(C3CCC(=O)NC3=O)C4=O)CC2)cc1)Cc1cc(C(F)(F)F)c2cn(-c3cccc([C@H](c4nncn4C)C4CCC4)c3)c(=O)n2c1 ; CN(CCOCc1cc(C(F)(F)F)c2cn(-c3cccc([C@H](c4nncn4C)C4CCC4)c3)c(=O)n2c1)C(=O)c1ccc(N2CCN(c3ccc4c(c3)CN([C@H]3CCC(=O)NC3=O)C4=O)CC2)cc1]

### Synthesis of Compound 148.

Compound 147-7 (400 mg) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 90% B to 90% B in 20 min; Wave Length: 220/254 nm; RT1 (min): 7.50; RT2 (min): 14.28; the second peak was product) to afford Compound 148 (121.0 mg, 30.25%) as a yellow solid.

LCMS-Compound 148: (ES, m/z): [M+H]⁺ 945

NMR-Compound 148: (400 MHz, DMSO, δ ppm): 61.69-1.81 (m, 5H), 1.90-1.98 (m, 1H), 2.02-2.11 (m, 1H), 2.33-2.40 (m, 1H), 2.50-2.64 (m, 1H), 2.82-3.01 (m, 4H), 3.15-3.24 (m, 1H), 3.35-3.48 (m, 11H), 3.57-3.62 (m, 4H), 4.20-4.37 (m, 5H), 5.04-5.08 (m, 1H), 6.97-7.03 (m, 3H), 7.10-7.21 (m, 3H), 7.29-7.35 (m, 3H), 7.42-7.46 (t, 1H), 7.54-7.56 (m, 1H), 7.70-7.80 (m, 3H), 8.32 (s, 1H), 10.96 (s, 1H).

### Example 147. Compound 149

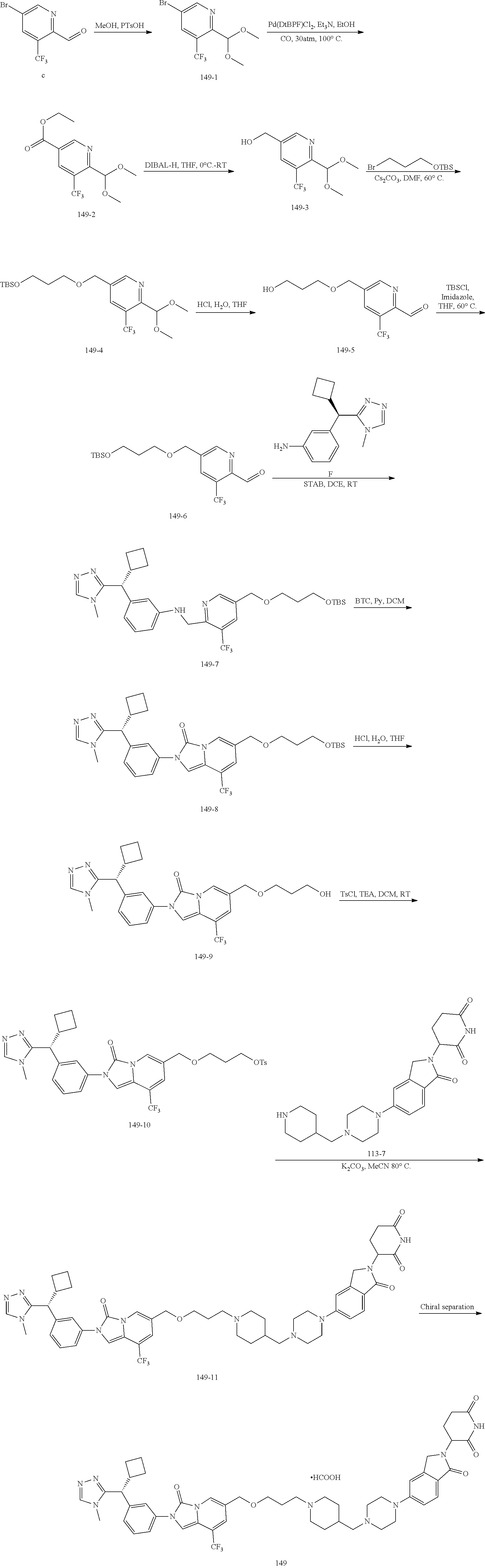

### Synthesis of Compound 149-1.

To a stirred solution of Intermediate C (60 g, 236.215 mmol, 1 equiv) in MEOH (600 mL) was added PTsOH (12.20 g, 70.864 mmol, 0.3 equiv) at room temperature. The resulting mixture was stirred overnight at 60° C. The resulting mixture was concentrated under vacuum. The reaction was quenched with NaHCO₃ (aq.) at room temperature. The aqueous layer was extracted with EtOAc (2×500 ml). The combined organic layers were washed with brine (500 mL), dried over anhydrous Na₂SO₄. After filtration, the filtrate was concentrated under reduced pressure. This resulted in Compound 149-1 (65 g, 85.28%) as a yellow solid.

### Synthesis of Compound 149-2.

To a solution of Compound 149-1 (65 g, 216.613 mmol, 1 equiv) and Et₃N (65.76 g, 649.839 mmol, 3 equiv) in EtOH (1000 mL) was added Pd(DtBPF)Cl₂ (14.12 g, 21.661 mmol, 0.1 equiv) in a pressure tank. The mixture was purged with nitrogen for 3 min and then was pressurized to 30 atm with carbon monoxide at 100° C. for overnight. The reaction mixture was cooled to room temperature and concentrated under vacuum. The resulting mixture was diluted with water (1 L). The aqueous layer was extracted with EtOAc (2×500 mL). The resulting mixture was concentrated under vacuum. The residue was purified by silica gel column chromatography, eluted with PE/EA (20:1) to afford Compound 149-2 (45 g, 67.30%) as a brown solid.

### Synthesis of Compound 149-3.

To a stirred solution of Compound 149-2 (45 g, 153.457 mmol, 1 equiv) in THF (450 mL) was added DIBAL-H (383.64 mL, 383.642 mmol, 2.5 equiv) dropwise at 0° C. under nitrogen atmosphere. The resulting mixture was stirred for 3 h at room temperature under nitrogen atmosphere. The reaction was quenched with NH₄Cl (aq.) (800 mL) at room temperature. The aqueous layer was extracted with EtOAc (2×300 mL). The resulting mixture was concentrated under vacuum. The residue was purified by silica gel column chromatography, eluted with DCM/MeOH (50:1) to afford Compound 149-3 (30 g, 74.71%) as a yellow oil.

### Synthesis of Compound 149-4.

To a stirred solution of Compound 149-3 (6 g, 23.885 mmol, 1 equiv) and (3-bromopropoxy)(tert-butyl)dimethylsilane (18.15 g, 71.655 mmol, 3 equiv) in DMF (100 mL) was added Cs₂CO₃ (23.35 g, 71.655 mmol, 3 equiv) at room temperature under nitrogen atmosphere. The resulting mixture was stirred overnight at 60° C. The resulting mixture was diluted with water (500 mL). The aqueous layer was extracted with EtOAc (2×200 mL). The resulting mixture was concentrated under vacuum. The residue was purified by silica gel column chromatography, eluted with PE/EA (10:1) to afford Compound 149-4 (8 g, 71.17%) as a light yellow oil.

### Synthesis of Compound 149-5.

To a stirred solution of Compound 149-4 (8 g, 18.888 mmol, 1 equiv) in THF (20 mL)/H₂O (50 mL) was added HCl (10 mL) at room temperature. The resulting mixture was stirred for overnight at 60° C. The mixture was basified to pH 8 with saturated NaHCO₃ (aq.). The aqueous layer was extracted with EtOAc (3×200 mL). The residue was purified by silica gel column chromatography, eluted with DCM/MeOH (100:1) to afford Compound 149-5 (5 g, 80.46%) as a light yellow oil.

### Synthesis of Compound 149-6.

To a stirred solution of Compound 149-5 (5 g, 18.996 mmol, 1 equiv) and TBSCl (5.73 g, 37.992 mmol, 2 equiv) in THF (100 mL) was added Imidazole (3.88 g, 56.988 mmol, 3 equiv) at room temperature under nitrogen atmosphere. The resulting mixture was stirred for 4 h at 60° C. The resulting mixture was diluted with water (200 mL). The aqueous layer was extracted with EtOAc (2×100 mL). The resulting mixture was concentrated under vacuum. The residue was purified by silica gel column chromatography, eluted with PE/EA (10:1) to afford Compound 149-6 (5 g, 64.85%) as a light yellow oil.

### Synthesis of Compound 149-7.

A solution of Compound 149-6 (5 g, 13.246 mmol, 1 equiv) and intermediate F (3.21 g, 13.246 mmol, 1 equiv) in DCE (100 mL) was stirred overnight at room temperature. To the above mixture was added STAB (5.61 g, 26.492 mmol, 2 equiv) at room temperature. The resulting mixture was stirred for additional 4 h at room temperature. The reaction was quenched with NaHCO₃ (aq.) (300 mL) at room temperature. The aqueous layer was extracted with DCM (2×100 mL). The resulting mixture was concentrated under vacuum. The residue was purified by silica gel column chromatography, eluted with DCM/MeOH (30:1) to afford Compound 149-7 (5 g, 60.64%) as a light yellow solid.

### Synthesis of Compound 149-8.

To a stirred solution of Compound 149-7 (5 g, 8.281 mmol, 1 equiv) and pyridine (6.55 g, 82.810 mmol, 10 equiv) in DCM (100 mL) was added triphosgene (0.98 g, 3.312 mmol, 0.4 equiv) at 0° C. The resulting mixture was stirred for 10 min at 0° C. The reaction was quenched with NaHCO₃ (aq.) (300 mL) at room temperature. The aqueous layer was extracted with DCM (3×100 mL). The resulting mixture was concentrated under vacuum. The crude product was used in the next step directly without further purification. This resulted in Compound 149-8 (4.5 g, 77.66%) as a yellow solid.

### Synthesis of Compound 149-9.

To a stirred solution of Compound 149-8 (4.5 g, 7.145 mmol, 1 equiv) in THF (10 mL)/H₂O (20 mL) was added conc. HCl (5 mL) at room temperature. The resulting mixture was stirred for 3 h at room temperature. The mixture was basified to pH 8 with saturated Na₂CO₃ (aq.). The aqueous layer was extracted with DCM (5×100 mL). The resulting mixture was concentrated under vacuum. The residue was purified by silica gel column chromatography, eluted with DCM/MeOH (20:1) to afford Compound 149-9 (3.5 g, 91.22%) as a yellow solid.

### Synthesis of Compound 149-10.

To a stirred solution of Compound 149-9 (3.5 g, 6.789 mmol, 1 equiv) and TsCl (2.59 g, 13.578 mmol, 2 equiv) in DCM (50 mL) was added TEA (2.06 g, 20.367 mmol, 3 equiv) at room temperature. The resulting mixture was stirred overnight at room temperature. The resulting mixture was concentrated under vacuum. The residue was purified by silica gel column chromatography, eluted with DCM/MeOH (30:1) to afford Compound 149-10 (2.5 g, 49.49%) as a yellow solid.

### Synthesis of Compound 149-11.

To a stirred solution of Compound 149-10 (1.5 g, 2.240 mmol, 1 equiv) and Compound 113-7 (0.95 g, 2.240 mmol, 1 equiv) in MeCN (30 ml) was added K₂CO₃ (0.62 g, 4.480 mmol, 2 equiv) at room temperature. The resulting mixture was stirred overnight at 80° C. The mixture was allowed to cool down to room temperature. The reaction was quenched with AcOH at room temperature. The resulting mixture was concentrated under vacuum. The residue was purified by reverse flash chromatography with the following conditions: column, C18 silica gel; mobile phase, MeCN in Water (0.1% FA), 0% to 40% gradient in 20 min; detector, UV 254 nm. The crude product was purified by Prep-HPLC with the following conditions (Column: Sunfire prep C18 column, 30*150 mm, 5 μm; Mobile Phase A: Water (0.1% FA), Mobile Phase B: ACN; Flow rate: 60 mL/min; Gradient: 14% B to 25% B in 7 min, Wave Length: 254; 220 nm; RT1 (min): 6.5) to afford Compound 149-11 (280 mg, 13.14%) as a yellow solid.

### Synthesis of Compound 149.

The Compound 149-11 (280 mg) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 60% B to 60% B in 15 min; Wave Length: 220/254 nm; RT1 (min): 7.75; RT2 (min): 11.73; the first peak is product) to afford the crude product. The crude product was purified by reverse flash chromatography with the following conditions: column, C18 silica gel; mobile phase, MeCN in water (0.1% FA), 10% to 50% gradient in 20 min; detector, UV 254 nm. This resulted in Compound 149 (82.5 mg, 27.19%) as a yellow solid.

LC-MS-Compound 149: (ES, m/z): [M−HCOOH+H]⁺ 923

H-NMR-Compound 149: (400 MHz, DMSO-d₆, δ ppm): 1.05-1.15 (m, 2H), 1.51-1.65 (m, 1H), 1.69-1.85 (m, 8H), 1.91-2.05 (m, 1H), 2.06-2.25 (m, 5H), 2.28-2.45 (m, 2H), 2.45-2.49 (m, 5H), 2.60-2.71 (m, 2H), 2.83-3.01 (m, 3H), 3.19-3.25 (m, 5H), 3.42-3.47 (m, 5H), 4.17-4.34 (m, 5H), 5.02-5.07 (d, 1H), 7.02-7.09 (m, 3H), 7.19-7.21 (d, 1H), 7.35 (s, 1H), 7.42-7.44 (m, 1H), 7.44-7.50 (m, 1H), 7.50-7.52 (d, 1H), 7.69-7.79 (m, 3H), 8.17 (s, 1H), 8.32 (s, 1H), 10.95 (s, 1H).

### Example 148. Compound 150

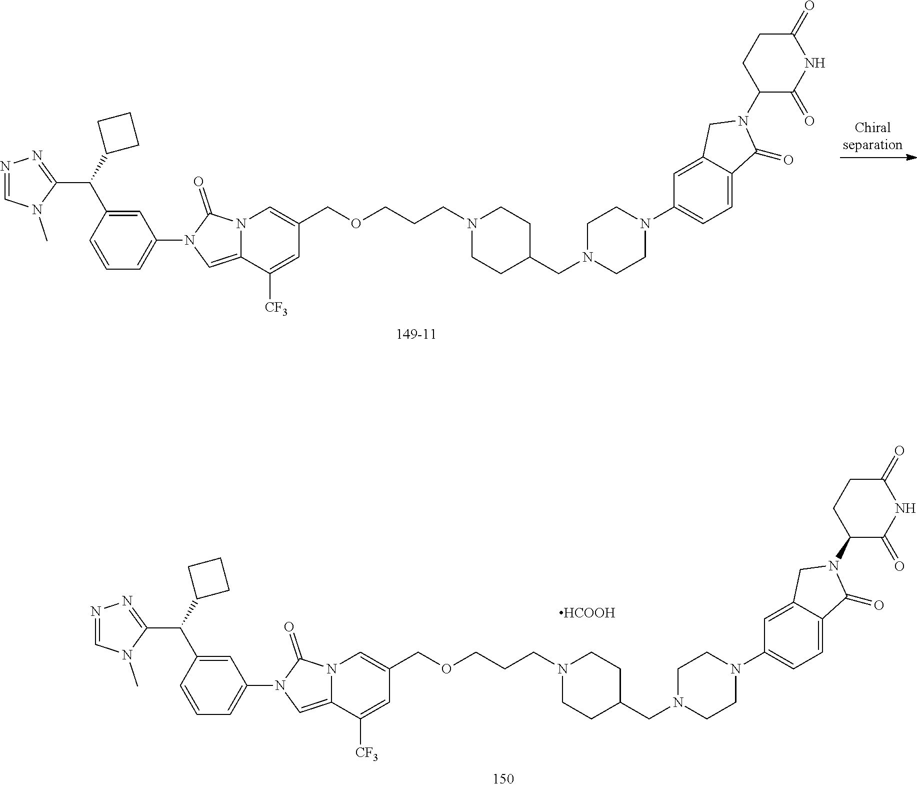 [SMILES: Cn1cnnc1[C@@H](c1cccc(-n2cc3c(C(F)(F)F)cc(COCCCN4CCC(CN5CCN(c6ccc7c(c6)CN(C6CCC(=O)NC6=O)C7=O)CC5)CC4)cn3c2=O)c1)C1CCC1 ; Cn1cnnc1[C@@H](c1cccc(-n2cc3c(C(F)(F)F)cc(COCCCN4CCC(CN5CCN(c6ccc7c(c6)CN([C@H]6CCC(=O)NC6=O)C7=O)CC5)CC4)cn3c2=O)c1)C1CCC1 ; "·HCOOH"]

### Synthesis of Compound 150.

The Compound 149-11 (280 mg, 0.303 mmol, 1 equiv) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 60% B to 60% B in 15 min; Wave Length: 220/254 nm; RT1 (min): 7.75; RT2 (min): 11.73; the second peak is product) to afford the crude product. The crude product was purified by reverse flash chromatography with the following conditions: column, C18 silica gel; mobile phase, MeCN in water (0.1% FA), 10% to 50% gradient in 20 min; detector, UV 254 nm. This resulted in Compound 150 (82.5 mg, 27.19%) as a yellow solid.

LC-MS-Compound 150: (ES, m/z): [M−HCOOH+H]⁺ 923

H-NMR-Compound 150: (400 MHz, DMSO-d₆, δ ppm): 1.05-1.15 (m, 2H), 1.51-1.65 (m, 1H), 1.69-1.85 (m, 8H), 1.91-2.13 (m, 4H), 2.13-2.15 (m, 2H), 2.28-2.49 (m, 7H), 2.60-2.71 (m, 2H), 2.83-2.98 (m, 3H), 3.15-3.32 (m, 5H), 3.42-3.47 (m, 5H), 4.17-4.34 (m, 1H), 5.02-5.07 (d, 1H), 7.02-7.09 (m, 3H), 7.18-7.20 (d, 1H), 7.35 (s, 1H), 7.42-7.44 (m, 1H), 7.44-7.50 (m, 1H), 7.50-7.52 (d, 1H), 7.69-7.79 (m, 3H), 8.18 (s, 1H), 8.32 (s, 1H), 10.95 (s, 1H).

### Example 149. Compound 151

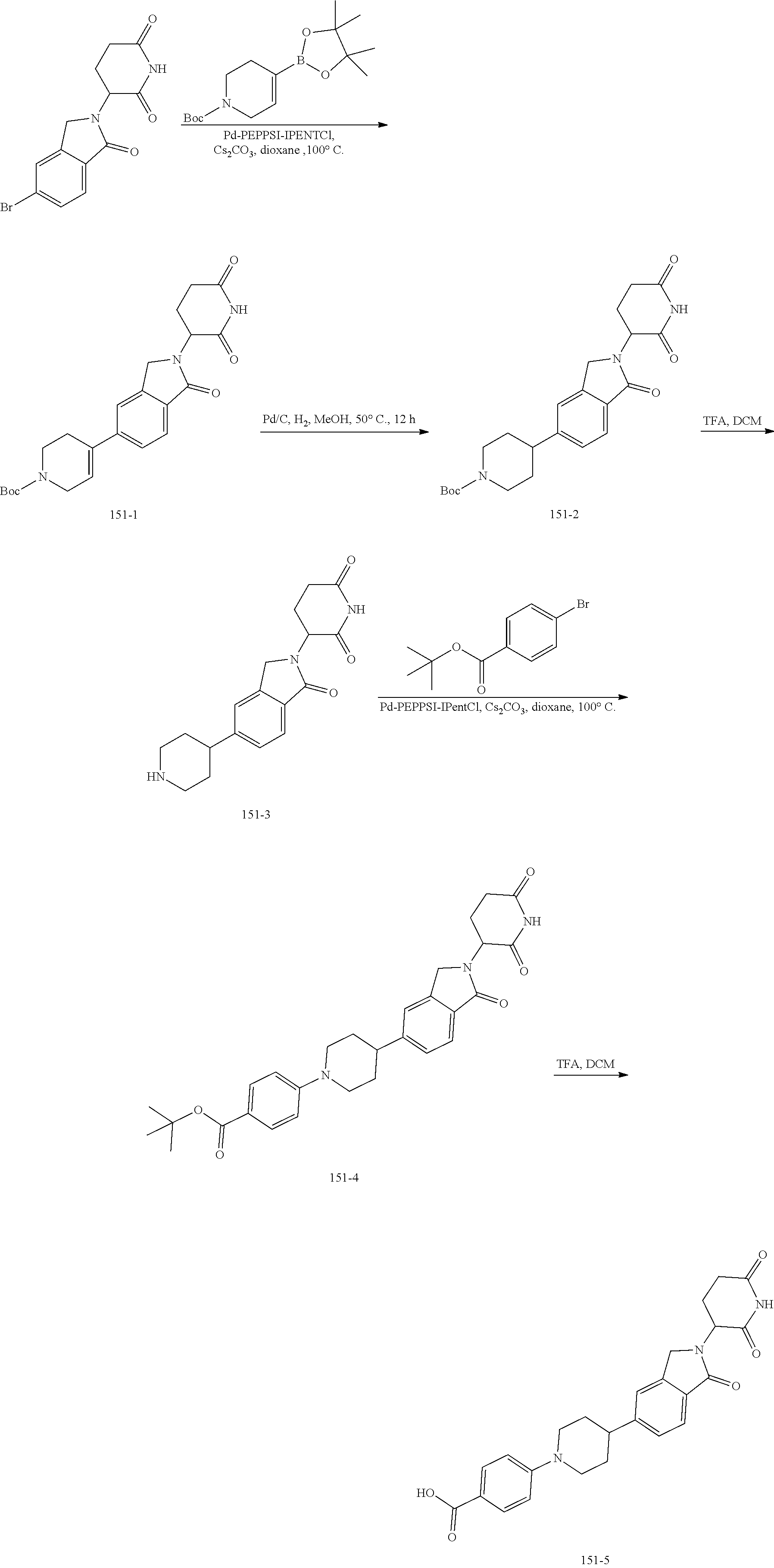

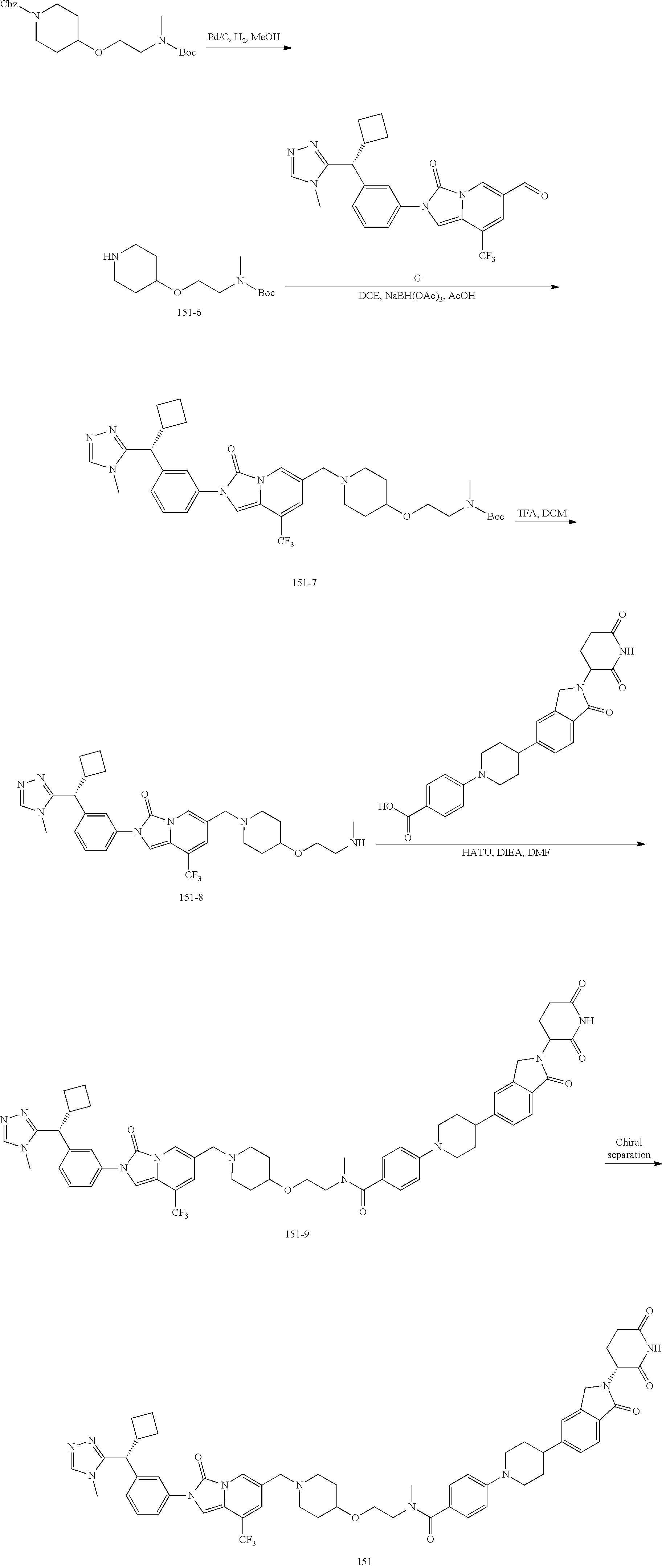

### Synthesis of Compound 151-1.

Into a 250 mL 3-necked round-bottom flask were added 3-(5-bromo-1-oxo-3H-isoindol-2-yl) piperidine-2,6-dione (10 g, 30.946 mmol, 1 equiv), dioxane (150 mL) tert-butyl 4-(4,4,5,5-tetramethyl-1,3,2-dioxaborolan-2-yl)-3,6-dihydro-2H-pyridine-1-carboxylate (19.14 g, 61.892 mmol, 2 equiv), Cs₂CO₃ (30.25 g, 92.838 mmol, 3 equiv) and Pd-PEPPSI-IPentCl (0.1.30 g, 1.547 mmol, 0.05 equiv) at room temperature. The resulting mixture was stirred for overnight at 100° C. under nitrogen atmosphere. The reaction was quenched by the addition of AcOH (10%) (150 mL) at room temperature. The aqueous layer was extracted with EtOAc (3×100 mL). The resulting mixture was concentrated under reduced pressure. The residue was purified by silica gel column chromatography, eluted with CH₂Cl₂/MeOH (12:1) to afford Compound 151-1 (3 g, 22.78%) as a red solid.

### Synthesis of Compound 151-2.

To a solution of Compound 151-1 (3 g, 7.051 mmol, 1 equiv) in 30 mL MeOH was added Pd/C (10%, 0.75 g) under nitrogen atmosphere in a 100 mL round-bottom flask. The mixture was hydrogenated at 50° C. for overnight under hydrogen atmosphere using a hydrogen balloon, filtered through a Celite pad and concentrated under reduced pressure to afford Compound 151-2 (2.3 g, 76.31%) as a light yellow solid.

### Synthesis of Compound 151-3.

Into a 100 mL round-bottom flask were added Compound 151-2 (2.3 g, 5.380 mmol, 1 equiv), DCM (21 mL) and TFA (7 mL) at room temperature. The resulting mixture was stirred for 1 h at room temperature. The resulting mixture was concentrated under reduced pressure. The residue was purified by Prep-TLC (CH₂Cl₂/MeOH 12:1) to afford Compound 151-3 (1.5 g, 85.16%) as a brown solid.

### Synthesis of Compound 151-4.

Into a 100 mL round-bottom flask were added Compound 151-3 (1.5 g, 4.582 mmol, 1 equiv), dioxane (20 mL), tert-butyl 4-bromobenzoate (1.18 g, 4.582 mmol, 1 equiv), Cs₂CO₃ (4.48 g, 13.746 mmol, 3 equiv) and Pd-PEPPSI-IPentCl (0.19 g, 0.229 mmol, 0.05 equiv) at room temperature. The resulting mixture was stirred for overnight at 100° C. under nitrogen atmosphere. The reaction was quenched by the addition of 10% AcOH in water (30 mL) at room temperature. The aqueous layer was extracted with EtOAc (3×30 mL). The resulting mixture was concentrated under reduced pressure. The residue was purified by silica gel column chromatography, eluted with CH₂Cl₂/MeOH (50:1) to afford Compound 151-4 (1 g, 43.34%) as a light yellow solid.

### Synthesis of Compound 151-5.

Into a 100 mL round-bottom flask were added Compound 151-4 (1 g, 1.986 mmol, 1 equiv), DCM (9 mL) and TFA (3 mL) at room temperature. The resulting mixture was stirred for 1 h at room temperature. The resulting mixture was concentrated under reduced pressure. The residue was purified by silica gel column chromatography, eluted with Cl₂C₂/MeOH (10:1) to afford Compound 151-5 (600 mg, 67.52%) as a brown solid.

### Synthesis of Compound 151-6.

To a solution of benzyl 4-{2-[tert-butoxycarbonyl) (methyl)amino] ethoxy} piperidine-1-carboxylate (5 g, 12.739 mmol, 1 equiv) in MeOH (150 mL) was added Pd/C (0.54 g, 5.096 mmol, 0.4 equiv) under nitrogen atmosphere in a 25 mL round-bottom flask. The mixture was hydrogenated at room temperature for 20 min under hydrogen atmosphere using a hydrogen balloon, filtered through a Celite pad and concentrated under reduced pressure to afford Compound 151-6 (3 g, 84.77%) as a yellow oil. The crude product was used in the next step directly without further purification.

### Synthesis of Compound 151-7.

Into an 8 mL sealed tube were added Compound 151-6 (1.5 g, 5.806 mmol, 1 equiv) DCM (20 mL), Intermediate G (2.64 g, 5.806 mmol, 1 equiv), NaBH(OAc)₃ (3.69 g, 17.418 mmol, 3 equiv) and AcOH (69.73 mg, 1.161 mmol, 0.2 equiv) at room temperature. A mixture was stirred for overnight at room temperature. The reaction was quenched with water (50 ml) at room temperature. The aqueous layer was extracted with EtOAc (3×50 mL). The resulting mixture was concentrated under reduced pressure. The residue was purified by silica gel column chromatography, eluted with CH₂Cl₂/MeOH (20:1) to afford Compound 151-7 (1.2 g, 27.84%) as a yellow solid.

### Synthesis of Compound 151-8.

Into a 50 mL 3-necked round-bottom flask were added Compound 151-7 (1.2 g, 1.720 mmol, 1 equiv), DCM (12 ml) and TFA (4 mL) at room temperature. A mixture was stirred for 4 h at room temperature. The reaction was quenched with sat. NaHCO₃ (aq.) (20 ml) at room temperature. The aqueous layer was extracted with EtOAc (3×10 mL). The resulting mixture was concentrated under reduced pressure. The residue was purified by Prep-TLC (CH₂Cl₂/MeOH 15:1) to afford Compound 151-8 (500 mg, 45.24%) as a yellow solid.

### Synthesis of Compound 151-9.

Into a 100 mL round-bottom flask were added Compound 151-8 (450 mg, 0.753 mmol, 1 equiv), DMF (10 mL), Compound 151-5 (336.92 mg, 0.753 mmol, 1 equiv), DIEA (291.93 mg, 2.259 mmol, 3 equiv) and HATU (429.42 mg, 1.129 mmol, 1.5 equiv) at room temperature. The resulting mixture was stirred for overnight at room temperature. The reaction was quenched by the addition of 10% AcOH in water (20 mL) at room temperature. The aqueous layer was extracted with EtOAc (3×20 mL). The resulting mixture was concentrated under reduced pressure. The residue was purified by Prep-TLC (CH₂Cl₂/MeOH 10:1) to afford Compound 151-9 (40 mg, 5.17%) as a white solid.

### Synthesis of Compound 151.

The Compound 151-9 (40 mg) was purified by Prep-Chiral-HPLC with the following conditions (Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 90% B to 90% B in 14 min; RT1 (min): 7.14; RT2 (min): 12.11; The first peak was the product. Sample Solvent: DCM:ACN=1:1—HPLC; Injection Volume: 0.8 mL; Number of Runs: 3) to afford crude product. The crude product was purified by reversed-phase flash chromatography with the following conditions: column, 40 g C18 silica gel; mobile phase, MeCN in water (0.1% FA), 20% to 50% gradient in 9 min; detector, UV 254 nm to afford Compound 151 (14.7 mg, 36.75%) as a light yellow solid.

LC-MS-Compound 151: (ES, m/z): [M−HCOOH+H]⁺ 1027

H-NMR-Compound 151: 1H NMR (400 MHz, DMSO-d6) δ 1.30-1.45 (m, 2H), 1.74-1.91 (m, 11H), 1.93-2.33 (m, 4H), 2.34-2.50 (m, 2H), 2.62-2.67 (m, 3H), 2.83-2.88 (m, 4H), 2.98 (s, 3H), 3.27 (s, 3H), 3.55 (s, 3H), 3.56-3.63 (m, 4H), 3.94-3.96 (d, 2H), 4.24-4.31 (m, 2H), 4.40-4.45 (m, 1H), 5.05-5.19 (m, 1H), 6.98-7.01 (t, 3H), 7.18-7.19 (d, 1H), 7.29-7.31 (d, 3H), 7.41-7.43 (d, 2H), 7.45 (s, 1H), 7.52-7.73 (m, 4H), 8.33 (s, 1H), 11.00 (s, 1H).

### Example 150. Compound 152

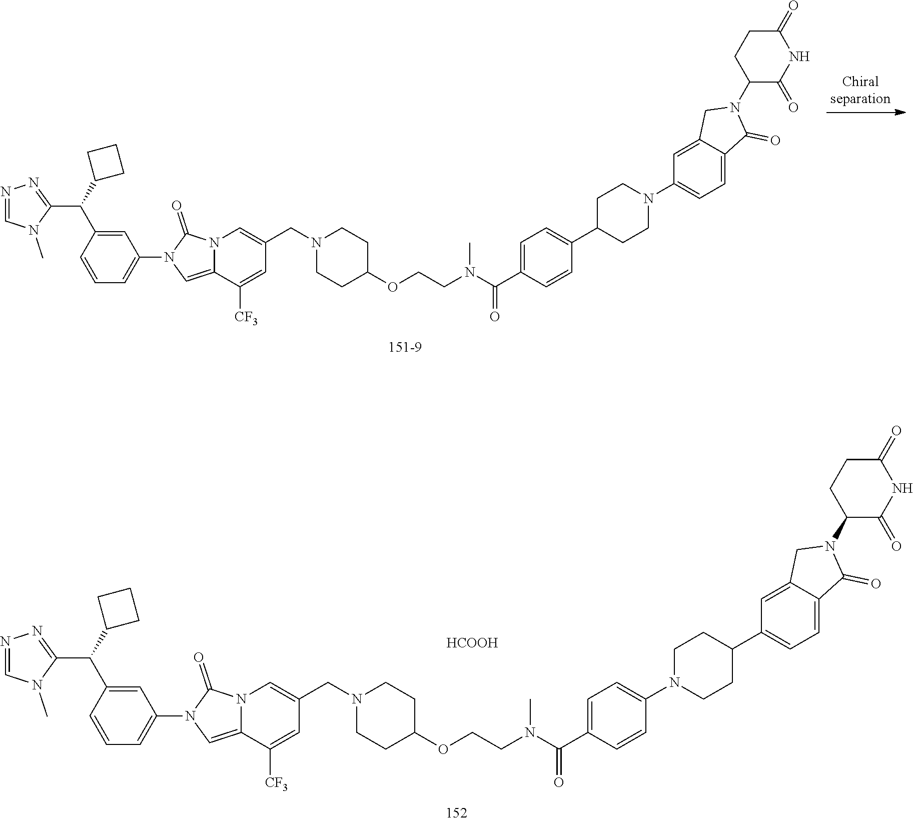 [SMILES: CN(CCOC1CCN(Cc2cc(C(F)(F)F)c3cn(-c4cccc([C@H](c5nncn5C)C5CCC5)c4)c(=O)n3c2)CC1)C(=O)c1ccc(C2CCN(c3ccc4c(c3)CN(C3CCC(=O)NC3=O)C4=O)CC2)cc1 ; CN(CCOC1CCN(Cc2cc(C(F)(F)F)c3cn(-c4cccc([C@H](c5nncn5C)C5CCC5)c4)c(=O)n3c2)CC1)C(=O)c1ccc(N2CCC(c3ccc4c(c3)CN([C@H]3CCC(=O)NC3=O)C4=O)CC2)cc1 ; "HCOOH"]

### Synthesis of Compound 152.

The Compound 151-9 (40 mg) was purified by Prep-Chiral-HPLC with the following conditions (Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 90% B to 90% B in 14 min; RT1 (min): 7.14; RT2 (min): 12.11; The second peak was the product. Sample Solvent: DCM:ACN=1:1—HPLC; Injection Volume: 0.8 mL; Number of Runs: 3 to afford crude product. The crude product was purified by reversed-phase flash chromatography with the following conditions: column, 40 g C18 silica gel; mobile phase, MeCN in water (0.1% FA), 20% to 55% gradient in 8 min; detector, UV 254 nm to afford Compound 152 (12.8 mg, 32.00%) as a light yellow solid.

LC-MS-Compound 152: (ES, m/z): [M−HCOOH+H]⁺ 1027

H-NMR-Compound 152: 1H NMR (400 MHz, DMSO-d6) δ 1.30-1.45 (m, 2H), 1.74-1.91 (m, 11H), 1.93-2.33 (m, 4H), 2.34-2.45 (m, 1H), 2.62-2.67 (m, 3H), 2.83-2.88 (m, 4H), 2.98 (s, 3H), 3.27 (s, 3H), 3.54-3.57 (m, 4H), 3.56-3.63 (m, 4H), 3.94-3.96 (d, 2H), 4.24-4.31 (m, 2H), 4.40-4.45 (m, 1H), 5.05-5.19 (m, 1H), 6.98-7.01 (t, 3H), 7.18-7.19 (d, 1H), 7.29-7.31 (d, 3H), 7.41-7.43 (d, 2H), 7.45 (s, 1H), 7.52-7.73 (m, 4H), 8.33 (s, 1H), 11.00 (s, 1H).

### Example 151. Compound 153

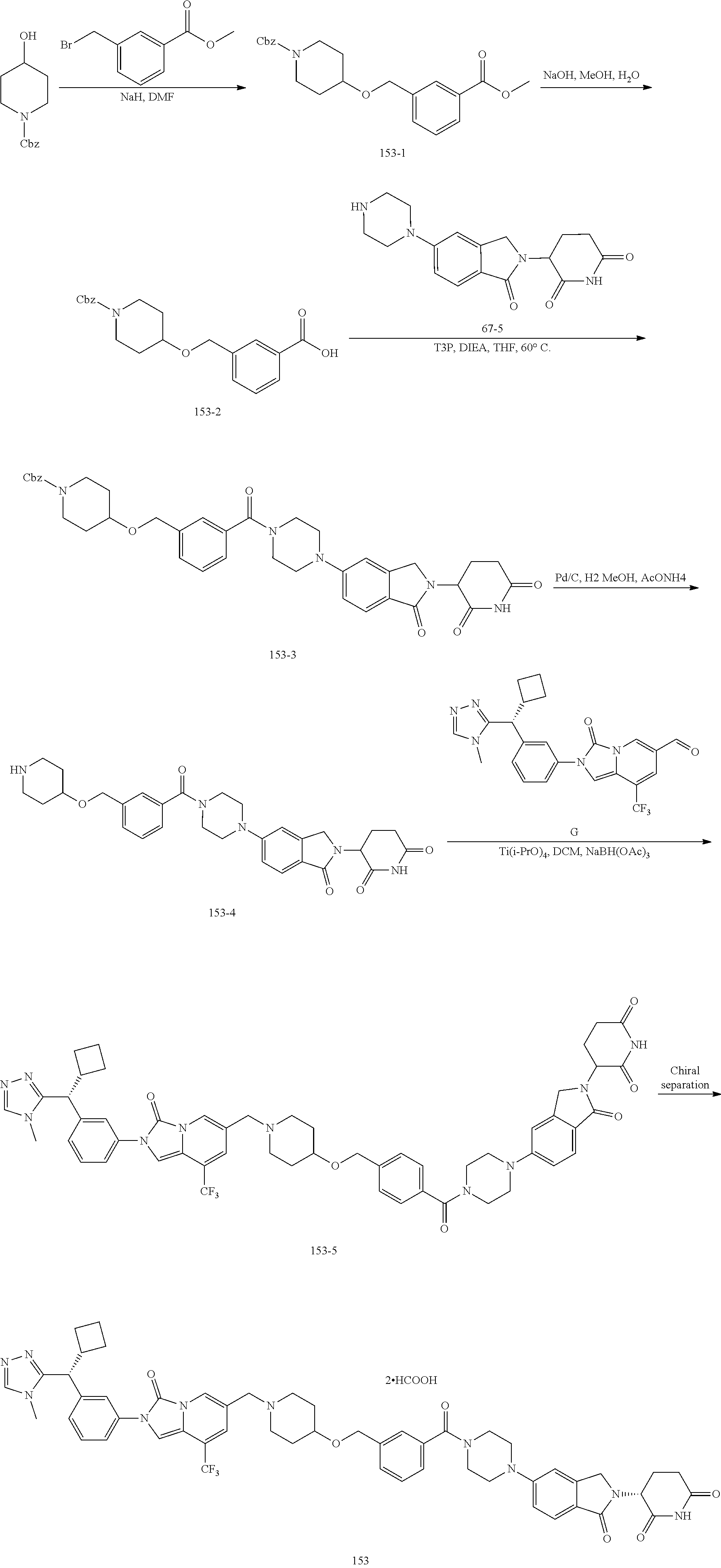

### Synthesis of Compound 153-1.

Into a 500 mL 3-necked round bottom flask were added benzyl 4-hydroxypiperidine-1-carboxylate (20 g, 85.004 mmol, 1 equiv), DMF (200 mL) and NaH (2.45 g, 102.005 mmol, 1.2 equiv) at 0° C. The resulting mixture was stirred for 1 h at 0° C. To the above mixture was added methyl 3-(bromomethyl)benzoate (38.94 g, 170.008 mmol, 2 equiv) at room temperature. The resulting mixture was stirred for overnight at room temperature. The reaction was quenched by the addition of sat. NH₄Cl (400 mL) at room temperature. The aqueous layer was extracted with EA (3×300 mL). The resulting mixture was concentrated under reduced pressure. The residue was purified by silica gel column chromatography, eluted with PE/EA (5:1) to afford Compound 153-1 (15 g, 46.02%) as a yellow oil.

### Synthesis of Compound 153-2.

Into a 500 mL 3-necked round bottom flask were added Compound 153-1 (15 g, 39.119 mmol, 1 equiv), MeOH (90 mL), H₂O (30 mL), THF (30 mL) and NaOH (4.69 g, 117.357 mmol, 3 equiv) at room temperature. The resulting mixture was stirred for 1 h at 50° C. The mixture was acidified to pH=5 with 1M HCl (100 mL). The aqueous layer was extracted with EA (3×100 mL). The resulting mixture was concentrated under reduced pressure. The residue was purified by silica gel column chromatography, eluted with PE/EA (5:1) to afford Compound 153-2 (12 g, 83.04%) as a white oil.

### Synthesis of Compound 153-3.

Into a 100 mL 3-necked round bottom flask were added Compound 153-2 (3 g, 5.612 mmol, 1 equiv), THF (30 mL), Compound 67-5 (2.21 g, 6.734 mmol, 1.2 equiv), DIEA (2.18 g, 16.836 mmol, 3 equiv) and T3P (5.36 g, 16.836 mmol, 3 equiv) at room temperature. The resulting mixture was stirred for overnight at 60° C. The reaction was quenched by the addition of H₂O (50 mL) at room temperature. The aqueous layer was extracted with DCM/MeOH=10:1 (3×50 mL). The resulting mixture was concentrated under reduced pressure. The residue was purified by silica gel column chromatography, eluted with DCM/MeOH (30:1) to afford Compound 153-3 (2 g, 52.43%) as a light yellow solid.

### Synthesis of Compound 153-4.

To a solution of Compound 153-3 (2 g, 2.942 mmol, 1 equiv) in 50 mL MeOH was added AcONH₄ (2.27 g, 29.420 mmol, 10 equiv). Pd/C (10%, 0.2 g) under nitrogen atmosphere in a 250 mL round bottom flask. The mixture was hydrogenated at room temperature for 2 h under hydrogen atmosphere using a hydrogen balloon, filtered through a Celite pad and concentrated under reduced pressure. This resulted in Compound 153-4 (1 g, 62.29%) as a light yellow solid.

### Synthesis of Compound 153-5.

Into a 50 mL 3-necked round bottom flask were added Compound 153-4 (1 g, 1.833 mmol, 1 equiv), DCM (10 mL), Intermediate G (0.83 g, 1.833 mmol, 1 equiv), TEA (0.19 g, 1.833 mmol, 1 equiv) and Ti(Oi-Pr)₄ (2.08 g, 7.332 mmol, 4 equiv) at room temperature. The resulting mixture was stirred for overnight at room temperature. To the above mixture was added NaBH(OAc)₃ (1.17 g, 5.499 mmol, 3 equiv) at room temperature. The resulting mixture was stirred for additional 2 h at room temperature. The reaction was quenched by the addition of water (100 mL) at room temperature. The aqueous layer was extracted with DCM/MeOH=10:1 (3×50 mL). The resulting mixture was concentrated under reduced pressure. The residue was purified by Prep-TLC (DCM/MeOH=10:1) to afford Compound 153-5 (400 mg, 22.16%) as a yellow solid.

### Synthesis of Compound 153.

The Compound 153-5 (400 mg) was purified by Prep-Chiral-HPLC with the following conditions (Column: CHIRALPAK IH, 2*25 cm, 5 μm; Mobile Phase A: Hex (0.2% TEA)—HPLC, Mobile Phase B: IPA:DCM=1:1—HPLC; Flow rate: 20 mL/min; Gradient: 70% B to 70% B in 12.5 min; Wave Length: 220/254 nm; RT1 (min): 7.17; RT2 (min): 10.25; The first peak was the product. Sample Solvent: IPA:DCM=1:1—HPLC; Injection Volume: 0.6 mL; Number of Runs: 12) to afford crude product. The crude product was purified by reversed-phase flash chromatography with the following conditions: column, 40 g C18 silica gel; mobile phase, MeCN in water (0.1% FA), 25% to 50% gradient in 8 min; detector, UV 254 nm to afford Compound 153 (69.2 mg, 16.78%) as a yellow solid.

LC-MS-Compound 153: (ES, m/z): [M−2HCOOH+H]⁺ 985

H-NMR-Compound 153: 1H NMR (300 MHz, DMSO-d₆) δ 1.54-1.57 (m, 2H), 1.74-1.81 (m, 5H), 1.85-1.88 (m, 3H), 2.05-2.10 (m, 1H), 2.21-2.23 (m, 2H), 2.50-2.52 (m, 1H), 2.60-2.62 (m, 1H), 2.70-2.73 (m, 2H), 2.90-2.91 (m, 1H), 3.17-3.19 (m, 2H), 3.22-3.26 (m, 4H), 3.32-3.43 (m, 6H), 3.46-3.50 (m, 1H), 3.75-3.78 (m, 2H), 4.20-4.37 (m, 3H), 4.56 (s, 2H), 5.04-5.08 (m, 1H), 7.03-7.05 (m, 1H), 7.08-7.10 (m, 2H), 7.18-7.20 (m, 1H), 7.33-7.37 (m, 3H), 7.40-7.45 (m, 3H), 7.47-7.55 (m, 1H), 7.57-7.74 (m, 3H), 8.34 (s, 1H), 10.98 (s, 1H).

### Example 152. Compound 154

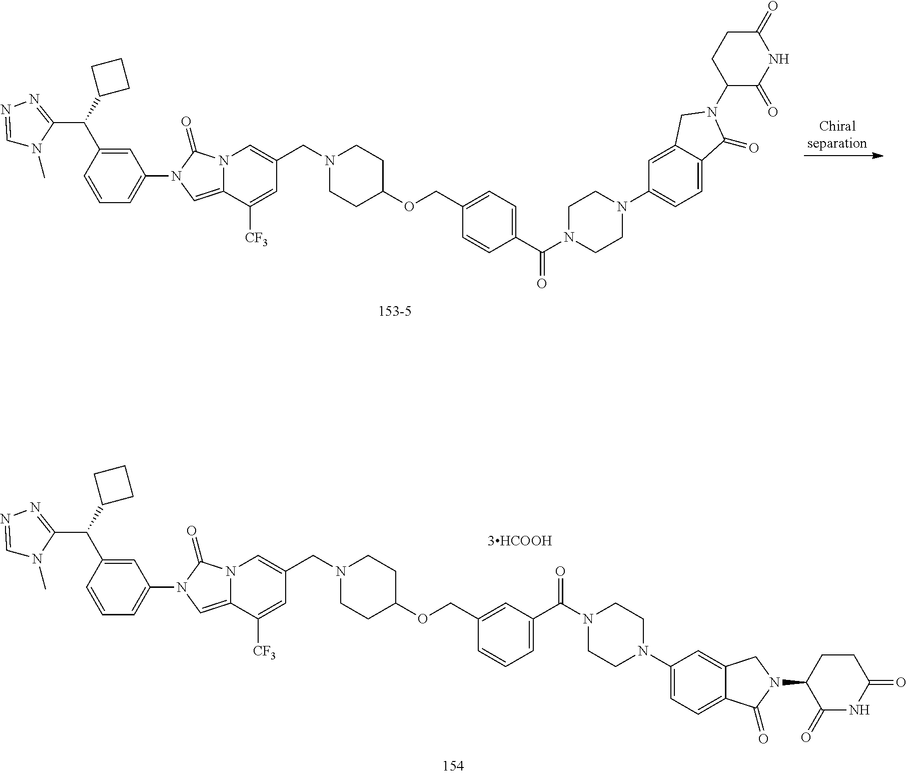 [SMILES: Cn1cnnc1[C@@H](c1cccc(-n2cc3c(C(F)(F)F)cc(CN4CCC(OCc5ccc(C(=O)N6CCN(c7ccc8c(c7)CN(C7CCC(=O)NC7=O)C8=O)CC6)cc5)CC4)cn3c2=O)c1)C1CCC1 ; Cn1cnnc1[C@@H](c1cccc(-n2cc3c(C(F)(F)F)cc(CN4CCC(OCc5cccc(C(=O)N6CCN(c7ccc8c(c7)CN([C@H]7CCC(=O)NC7=O)C8=O)CC6)c5)CC4)cn3c2=O)c1)C1CCC1]

### Synthesis of Compound 154.

The Compound 153-5 (400 mg) was purified by Prep-Chiral-HPLC with the following conditions (Column: CHIRALPAK IH, 2*25 cm, 5 μm; Mobile Phase A: Hex (0.2% TEA)—HPLC, Mobile Phase B: IPA:DCM=1:1—HPLC; Flow rate: 20 mL/min; Gradient: 70% B to 70% B in 12.5 min; Wave Length: 220/254 nm; RT1 (min): 7.17; RT2 (min): 10.25; The second peak was the product. Sample Solvent: IPA:DCM=1:1—HPLC; Injection Volume: 0.6 mL; Number of Runs: 12) to afford crude product. The crude product was purified by reversed-phase flash chromatography with the following conditions: column, 40 g C18 silica gel; mobile phase, MeCN in water (0.1% FA), 25% to 50% gradient in 8 min; detector, UV 254 nm to afford Compound 154 (103.4 mg, 25.18%) as a yellow solid.

LC-MS-Compound 154: (ES, m/z): [M−3HCOOH+H]⁺ 985

H-NMR-Compound 154: 1H NMR (300 MHz, DMSO-d₆) δ 1.54-1.58 (m, 2H), 1.78-1.81 (m, 5H), 1.82-1.88 (m, 3H), 2.04-2.09 (m, 1H), 2.21-2.23 (m, 2H), 2.50-2.52 (m, 2H), 2.60-2.62 (m, 1H), 2.72-2.74 (m, 2H), 2.90-2.92 (m, 1H), 3.19-3.23 (m, 2H), 3.25-3.27 (m, 3H), 3.32-3.47 (m, 6H), 3.60-3.64 (m, 1H), 3.75-3.77 (m, 2H), 4.19-4.36 (m, 3H), 4.56 (s, 2H), 5.04-5.07 (m, 1H), 7.03-7.05 (m, 1H), 7.08-7.10 (m, 2H), 7.19-7.21 (m, 1H), 7.33-7.37 (m, 2H), 7.40-7.45 (m, 4H), 7.46-7.55 (m, 1H), 7.57-7.74 (m, 3H), 8.34 (s, 1H), 10.98 (s, 1H).

### Example 153. Compound 155

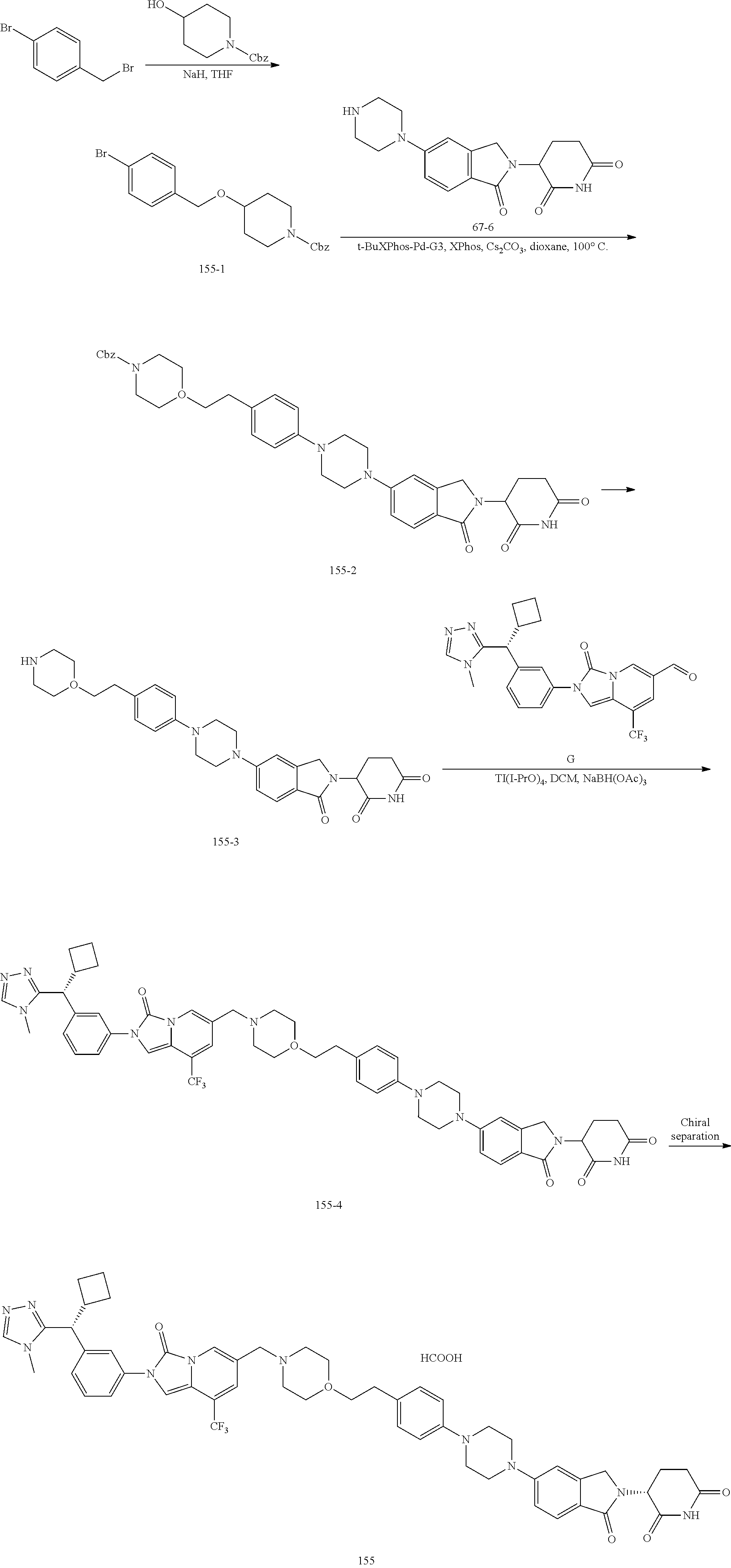

### Synthesis of Compound 155-1.

A solution of benzyl 4-hydroxypiperidine-1-carboxylate (15 g, 63.75 mmol, 1.0 equiv) in THF (150 mL) was treated with NaH (3, g, 127.50 mmol, 2.0 equiv) for 1 h at 0° C. under nitrogen atmosphere followed by the addition of 1-bromo-4-(bromomethyl)benzene (16 g, 63.75 mmol, 1.0 equiv) at room temperature. The resulting mixture was stirred for overnight at room temperature under nitrogen atmosphere. The reaction was quenched by the addition of sat. NH₄Cl (aq.) (300 mL) at room temperature. The aqueous layer was extracted with EtOAc (3×200 mL). The residue was purified by silica gel column chromatography, eluted with PE/EA (10:1) to afford Compound 155-1 (22 g, 78%) as a yellow oil.

### Synthesis of Compound 155-2.

To a stirred solution of benzyl Compound 155-1 (5 g, 12.37 mmol, 1.0 equiv) and Compound 67-6 (4.5 g, 13.60 mmol, 1.1 equiv) in 1,4-dioxane (50 mL) were added Pd PEPPSI IPentC (0.4 g, 1.237 mmol, 0.1 equiv) and Cs₂CO₃ (31.93 g, 37.101 mmol, 3 equiv) at room temperature under nitrogen atmosphere. The resulting mixture was stirred for overnight at 100° C. under nitrogen atmosphere. The reaction was quenched by the addition of 10% AcOH (500 mL) at room temperature. The aqueous layer was extracted with CH₂Cl₂:MeOH (5:1) (3×400 mL). The resulting mixture was concentrated under reduced pressure. The residue was purified by silica gel column chromatography, eluted with CH₂Cl₂/MeOH (20:1) to afford Compound 155-2 (2 g, 25%) as an off-white solid.

### Synthesis of Compound 155-3.

To a solution of benzyl Compound 155-2 (1.2 g, 1.78 mmol, 1.0 equiv) and CH₃COONH₄ (1.4 g, 17.80 mmol, 10.0 equiv) in 34 mL MeOH was added Pd/C (10%, 340 mg) under nitrogen atmosphere in a 100 mL round-bottom flask. The mixture was hydrogenated at room temperature for 2 h under hydrogen atmosphere using a hydrogen balloon, filtered through a Celite pad and concentrated under reduced pressure. The residue was purified by reversed-phase flash chromatography with the following conditions: column, C18 silica gel; mobile phase, MeCN in water (0.1% FA), 10% to 50% gradient in 25 min; detector, UV 254 nm. This resulted in Compound 155-3 (600 mg, 65%) as an off-white solid.

### Synthesis of Compound 155-4.

To a stirred solution of Compound 155-3 (580 mg, 1.12 mmol, 1.0 equiv) and Intermediate G (612 mg, 1.34 mmol, 1.2 equiv) in DCM (6 mL) was added Ti(Oi-Pr)₄ (1273 mg, 4.48 mmol, 4.0 equiv) at room temperature. The resulting mixture was stirred for overnight at room temperature. To the above mixture was added NaBH(OAc)₃ (712 mg, 3.36 mmol, 3.0 equiv) at room temperature. The resulting mixture was stirred for additional 2 h at room temperature. The reaction was quenched by the addition of 10% AcOH (100 mL) at room temperature. The aqueous layer was extracted with EtOAc (2×60 mL). The aqueous phase was concentrated under reduced pressure. The residue was purified by reversed-phase flash chromatography with the following conditions: column, C18 silica gel; mobile phase, MeCN in water (0.1% FA), 10% to 50% gradient in 30 min; detector, UV 254 nm. The crude product (600 mg) was purified by Prep-HPLC with the following conditions (Column: Xselect CSH C18 OBD Column 30*150 mm 5 μm, n; Mobile Phase A: water (0.1% FA), Mobile Phase B: ACN; Flow rate: 60 mL/min; Gradient: 23% B to 35% B in 7 min, 35% B; Wave Length: 254/220 nm; RT1 (min): 6.23; Number of Runs: 0) to afford Compound 155-4 (580 mg, 54%) as a yellow solid.

### Synthesis of Compound 155.

The Compound 155-4 (260 mg) was purified by Prep-CHIRAL-HPLC with the following conditions (Prep-HPLC-037): Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 60% B to 60% B in 14 min; Wave Length: 220/254 nm; RT1 (min): 7.52; RT2 (min): 11.59; Sample Solvent: DCM:ACN=1:1—HPLC; Injection Volume: 1.3 mL; Number Of Runs: 5. The first peak was the product. The residue was purified by reversed-phase flash chromatography with the following conditions: column, C18 silica gel; mobile phase, MeCN in water (0.1% FA), 10% to 50% gradient in 30 min; detector, UV 254 nm. This resulted in Compound 155 (86 mg, 31%) as a yellow solid.

LC-MS-Compound 155: (ES, m/z): [M-COOH]⁺ 957

H-NMR-Compound 155: (400 MHz, DMSO-d6, δ ppm): 1.50-1.58 (m, 2H), 1.76-1.81 (m, 7H), 1.92-1.96 (m, 1H), 2.05-2.14 (m, 3H), 2.31-2.41 (m, 1H), 2.50-2.60 (m, 2H), 2.67-2.69 (m, 2H), 2.85-2.92 (m, 1H), 3.22-3.29 (m, 7H), 3.36-3.44 (m, 7H), 4.24-4.39 (m, 5H), 5.03-5.06 (m, 1H), 6.96-7.01 (m, 3H), 7.12-7.18 (m, 2H), 7.19-7.21 (m, 3H), 7.30 (s, 1H), 7.41-7.45 (m, 1H), 7.54-7.56 (d, 1H), 7.65-7.73 (m, 3H), 8.18 (s, 1H), 8.32 (s, 1H), 10.95 (s, 1H).

### Example 154. Compound 156

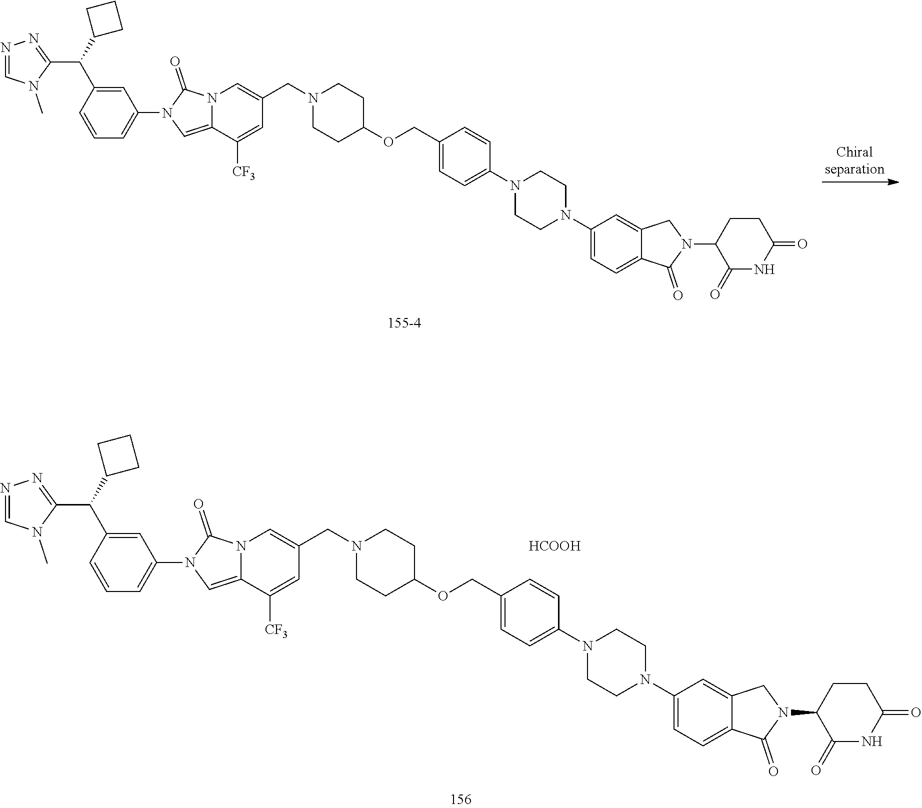 [SMILES: Cn1cnnc1[C@@H](c1cccc(-n2cc3c(C(F)(F)F)cc(CN4CCC(OCc5ccc(N6CCN(c7ccc8c(c7)CN(C7CCC(=O)NC7=O)C8=O)CC6)cc5)CC4)cn3c2=O)c1)C1CCC1 ; Cn1cnnc1[C@@H](c1cccc(-n2cc3c(C(F)(F)F)cc(CN4CCC(OCc5ccc(N6CCN(c7ccc8c(c7)CN([C@H]7CCC(=O)NC7=O)C8=O)CC6)cc5)CC4)cn3c2=O)c1)C1CCC1 ; "HCOOH"]

### Synthesis of Compound 156.

The Compound 155-4 (260 mg) was purified by Prep-CHIRAL-HPLC with the following conditions (Prep-HPLC-037): Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 60% B to 60% B in 14 min; Wave Length: 220/254 nm; RT1 (min): 7.52; RT2 (min): 11.59; Sample Solvent: DCM:ACN=1:1—HPLC; Injection Volume: 1.3 mL; Number Of Runs: 5. The second peak was the product. The residue was purified by reversed-phase flash chromatography with the following conditions: column, C18 silica gel; mobile phase, MeCN in water (0.1% FA), 10% to 50% gradient in 30 min; detector, UV 254 nm. This resulted in Compound 156 (85 mg, 31%) as a yellow solid.

LC-MS-Compound 156: (ES, m/z): [M-COOH]⁺ 957

H-NMR-Compound 156: (400 MHz, DMSO-d6, δ ppm): 1.50-1.58 (m, 2H), 1.76-1.81 (m, 7H), 1.92-1.96 (m, 1H), 2.05-2.14 (m, 3H), 2.31-2.41 (m, 1H), 2.50-2.60 (m, 2H), 2.67-2.69 (m, 2H), 2.85-2.92 (m, 1H), 3.22-3.29 (m, 7H), 3.36-3.44 (m, 7H), 4.24-4.39 (m, 5H), 5.03-5.06 (m, 1H), 6.96-7.01 (m, 3H), 7.12-7.18 (m, 2H), 7.19-7.21 (m, 3H), 7.30 (s, 1H), 7.41-7.45 (m, 1H), 7.54-7.56 (d, 1H), 7.65-7.73 (m, 3H), 8.18 (s, 1H), 8.32 (s, 1H), 10.95 (s, 1H).

### Example 155. Compound 157

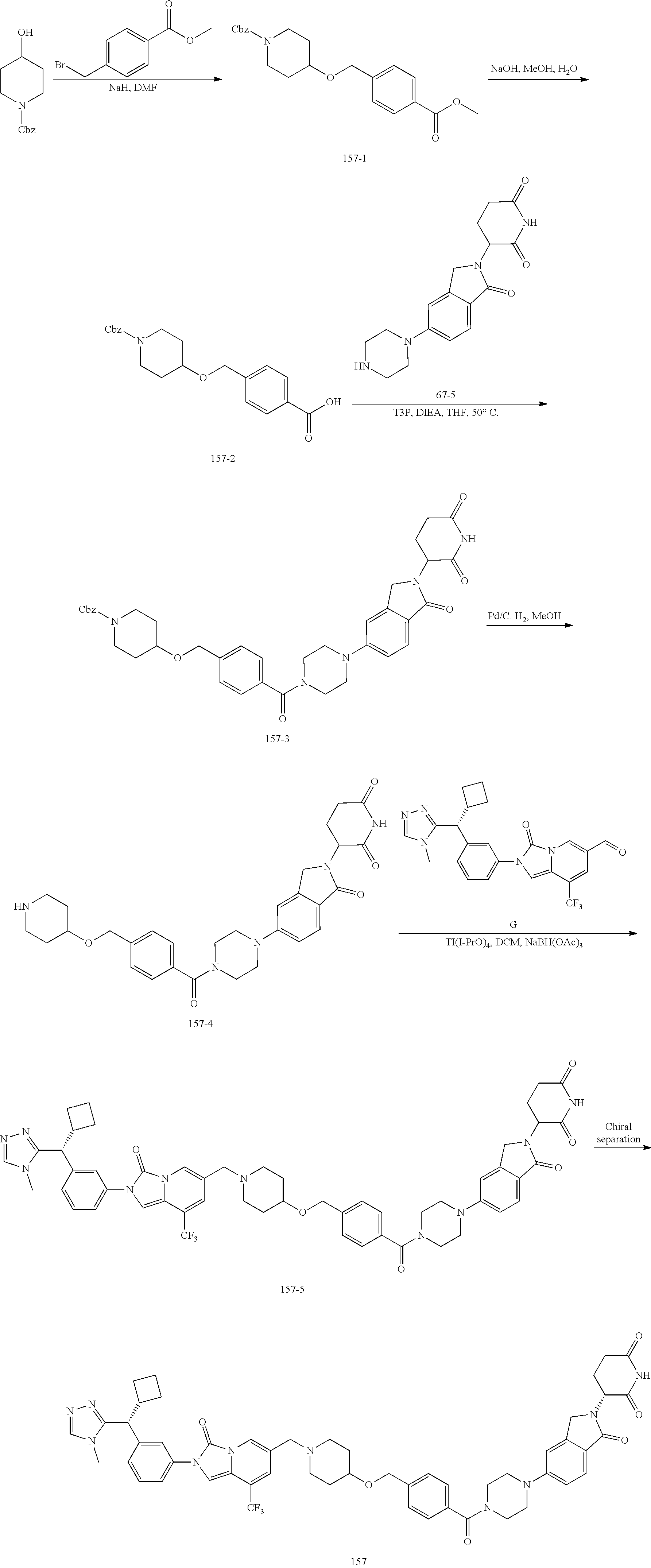

### Synthesis of Compound 157-1.

Into a 250 mL 3-necked round-bottom flask were added benzyl 4-hydroxypiperidine-1-carboxylate (12 g, 51.002 mmol, 1 equiv) and DMF (150 mL) at room temperature. To the above mixture was added NaH (1.47 g, 61.202 mmol, 1.2 equiv) at 0° C. The resulting mixture was stirred for additional 30 min at 0° C. To the above mixture was added methyl 4-(bromomethyl) benzoate (23.37 g, 102.004 mmol, 2 equiv) at 0° C. The resulting mixture was stirred for additional overnight at room temperature. The reaction was quenched by the addition of sat. NH₄Cl (aq.) (150 mL) at room temperature. The resulting mixture was extracted with EtOAc (3×200 mL). The combined organic layers were concentrated under reduced pressure. The residue was purified by silica gel column chromatography, eluted with CH₂Cl₂/MeOH (80:1) to afford Compound 157-1 (6 g, 29.15%) as a light yellow oil.

### Synthesis of Compound 157-2.

Into a 250 mL 3-necked round-bottom flask were added NaOH (1.88 g, 46.944 mmol, 3 equiv), MeOH (36 mL) and H₂O (12 mL) at room temperature. To the above mixture was added Compound 157-1 (6 g, 15.648 mmol, 1 equiv) at room temperature. The resulting mixture was stirred for 5 h at room temperature. The reaction was quenched by the addition of sat. NH₄Cl (aq.) (100 mL) at room temperature. The resulting mixture was extracted with CH₂Cl₂ (3×150 mL). The combined organic layers were concentrated under reduced pressure. The residue was purified by silica gel column chromatography, eluted with CH₂Cl₂/MeOH (30:1) to afford Compound 157-2 (4.5 g, 73.96%) as a light yellow oil.

### Synthesis of Compound 157-3.

Into a 100 mL 3-necked round-bottom flask were added Compound 157-2 (2.5 g, 6.767 mmol, 1 equiv), THF (25 mL), Compound 67-5 (2.22 g, 6.767 mmol, 1 equiv), DIEA (2.62 g, 20.301 mmol, 3 equiv) and T₃P (6.46 g, 20.301 mmol, 3 equiv) at room temperature. The resulting mixture was stirred for 4 h at 60° C. under nitrogen atmosphere. The reaction was quenched by the addition of sat. NH₄Cl (aq.) (50 mL) at room temperature. The resulting mixture was extracted with CH₂Cl₂ (3×60 mL). The combined organic layers were concentrated under reduced pressure. The residue was purified by reversed-phase flash chromatography with the following conditions: column, C18 silica gel; mobile phase, MeCN in water (0.1% FA), 10% to 50% gradient in 10 min; detector, UV 254 min. This resulted in Compound 157-3 (4 g, 82.60%) as a light yellow oil.

### Synthesis of Compound 157-4.

To a solution of Compound 157-3 (3 g, 4.413 mmol, 1 equiv) in 60 mL MeOH was added Pd/C (10%, 0.3 g) under nitrogen atmosphere in a 250 mL round bottom flask. The mixture was hydrogenated at room temperature for 2 h under hydrogen atmosphere using a hydrogen balloon, filtered through a Celite pad and concentrated under reduced pressure. The residue was purified by reversed-phase flash chromatography with the following conditions: column, C18 silica gel; mobile phase, MeCN in water (0.1% FA), 10% to 50% gradient in 10 min; detector, UV 254 nm. This resulted in Compound 157-4 (1.5 g, 59.18%) as a light yellow oil.

### Synthesis of Compound 157-5.

Into a 100 mL 3-necked round-bottom flask were added Compound 157-4 (1 g, 1.833 mmol, 1 equiv), DCM (10 mL), Intermediate G (0.83 g, 1.833 mmol, 1 equiv) and Ti(Oi-Pr)₄ (2.08 g, 7.332 mmol, 4 equiv) at room temperature. The resulting mixture was stirred for overnight at room temperature under nitrogen atmosphere. To the above mixture was added NaBH(OAc)₃ (1.17 g, 5.499 mmol, 3 equiv) at room temperature. The resulting mixture was stirred for additional 3 h at room temperature. The reaction was quenched by the addition of AcOH (aq.) (10%) (20 mL) at room temperature. The resulting mixture was extracted with CH₂Cl₂ (3×30 mL). The combined organic layers were concentrated under reduced pressure. The residue was purified by reversed-phase flash chromatography with the following conditions: column, C18 silica gel; mobile phase, MeCN in water (0.1% FA), 10% to 50% gradient in 10 min; detector, UV 254 nm. This resulted in Compound 157-5 (500 mg, 26.03%) as a light yellow oil.

### Synthesis of Compound 157.

The Compound 157-5 (240 mg) was purified by Prep-Chiral-HPLC with the following conditions (Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 90% B to 90% B in 30 min; Wave Length: 220/254 nm; RT1 (min): 8.81; RT2 (min): 22.23; The first peak was the product. Sample Solvent: DCM:ACN=1:1—HPLC; Injection Volume: 1.55 mL; Number of Runs: 2) to afford Compound 157 (70.5 mg, 22.32%) as a yellow solid.

LCMS-Compound 157: (ES, m/z): [M+H]⁺ 985

H-NMR-Compound 157: (300 MHz, DMSO-d6, ppm): δ1.54-1.56 (m, 2H), δ1.74-1.80 (m, 5H), δ1.88-2.00 (m, 3H), δ2.00-2.18 (m, 3H), δ2.36-2.50 (m, 2H), δ2.51-2.60 (m, 1H), δ2.60-2.71 (m, 2H), δ2.71-2.90 (m, 1H), δ3.00-3.25 (m, 3H), δ3.34-3.43 (m, 4H), δ3.46-3.50 (m, 4H), δ3.69-3.82 (m, 2H), δ4.19-4.25 (m, 2H), δ4.27-4.36 (m, 2H), δ4.56 (m, 2H), δ5.03-5.08 (m, 1H), δ7.02-7.07 (m, 1H), δ7.10-7.18 (m, 2H), δ7.18-7.20 (m, 1H), δ7.20-7.32 (m, 1H), δ7.40-7.54 (m, 5H), δ7.54-7.56 (m, 1H) δ7.67-7.73 (m, 3H), δ8.33 (s, 1H), δ10.96 (s, 1H).

### Example 156. Compound 158

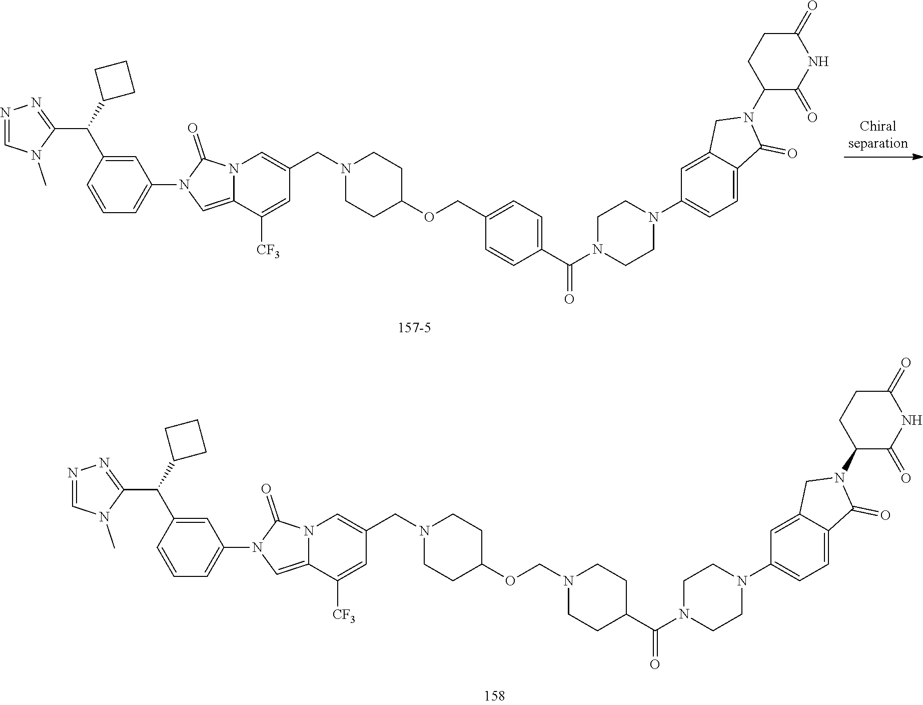 [SMILES: Cn1cnnc1[C@@H](c1cccc(-n2cc3c(C(F)(F)F)cc(CN4CCC(OCc5ccc(C(=O)N6CCN(c7ccc8c(c7)CN(C7CCC(=O)NC7=O)C8=O)CC6)cc5)CC4)cn3c2=O)c1)C1CCC1 ; Cn1cnnc1[C@@H](c1cccc(-n2cc3c(C(F)(F)F)cc(CN4CCC(OCN5CCC(C(=O)N6CCN(c7ccc8c(c7)CN([C@H]7CCC(=O)NC7=O)C8=O)CC6)CC5)CC4)cn3c2=O)c1)C1CCC1]

### Synthesis of Compound 158.

The Compound 157-5 (240 mg) was purified by Prep-Chiral-HPLC with the following conditions (Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 90% B to 90% B in 30 min; Wave Length: 220/254 nm; RT1 (min): 8.81; RT2 (min): 22.23; The second peak was the product. Sample Solvent: DCM:ACN=1:1—HPLC; Injection Volume: 1.55 mL; Number of Runs: 2) to afford Compound 158 (34.5 mg, 22.32%) as a yellow solid.

LCMS-Compound 158: (ES, m/z): [M+H]⁺ 985

H-NMR-Compound 158: (300 MHz, DMSO-d6, ppm): δ1.53-1.56 (m, 2H), δ1.74-1.79 (m, 5H), δ1.80-2.00 (m, 3H), δ2.00-2.25 (m, 3H), δ2.36-2.50 (m, 2H), δ2.51-2.60 (m, 1H), δ2.60-2.71 (m, 2H), δ2.84-2.95 (m, 1H), δ3.05-3.29 (m, 2H), δ3.40-3.43 (m, 3H), δ3.45-3.65 (m, 6H), δ3.75-3.85 (m, 2H), δ4.19-4.25 (m, 2H), δ4.27-4.36 (m, 2H), δ4.56 (m, 2H), δ5.03-5.08 (m, 1H), δ7.02-7.07 (m, 1H), δ7.10-7.20 (m, 2H), δ7.20-7.32 (m, 1H), δ7.32-7.42 (m, 1H), δ7.42-7.56 (m, 6H), δ7.67-7.68 (m, 1H), δ7.68-7.70 (m, 1H), δ7.70-7.73 (m, 1H), δ8.33 (s, 1H), δ10.96 (s, 1H).

### Example 157. Compound 159

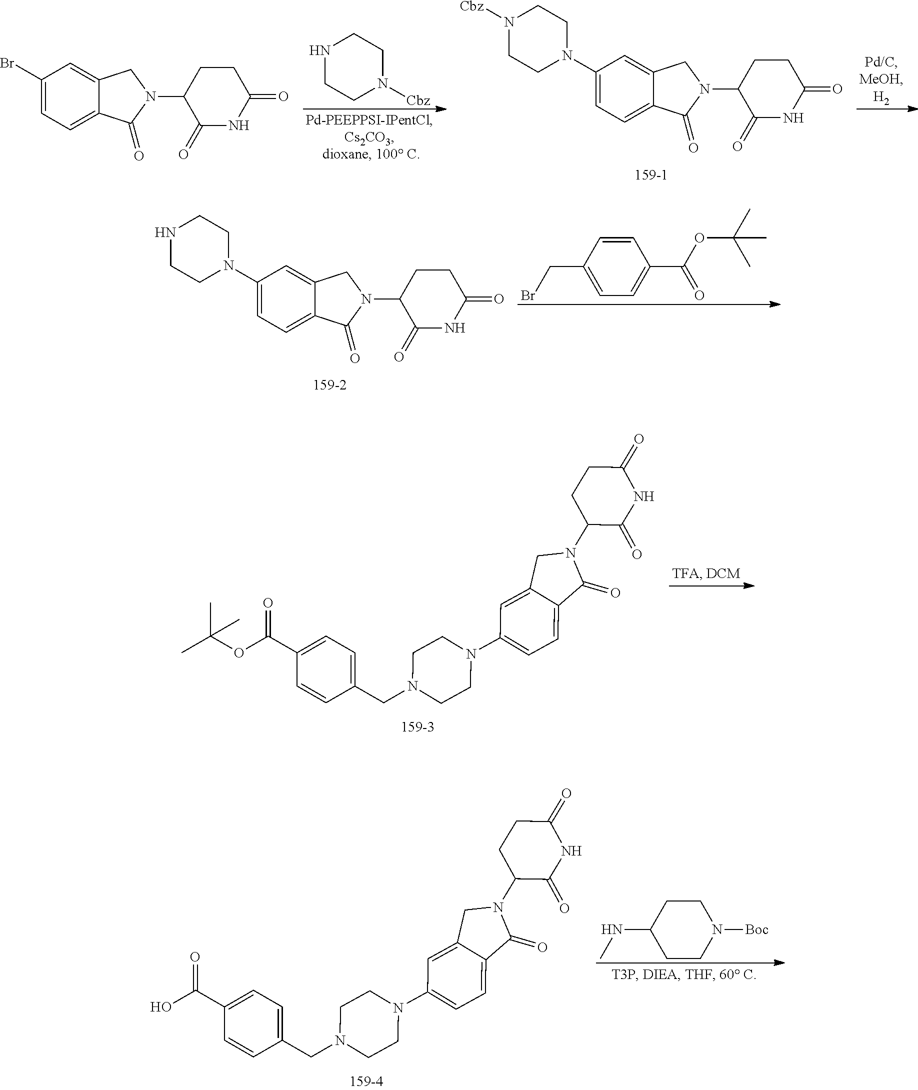

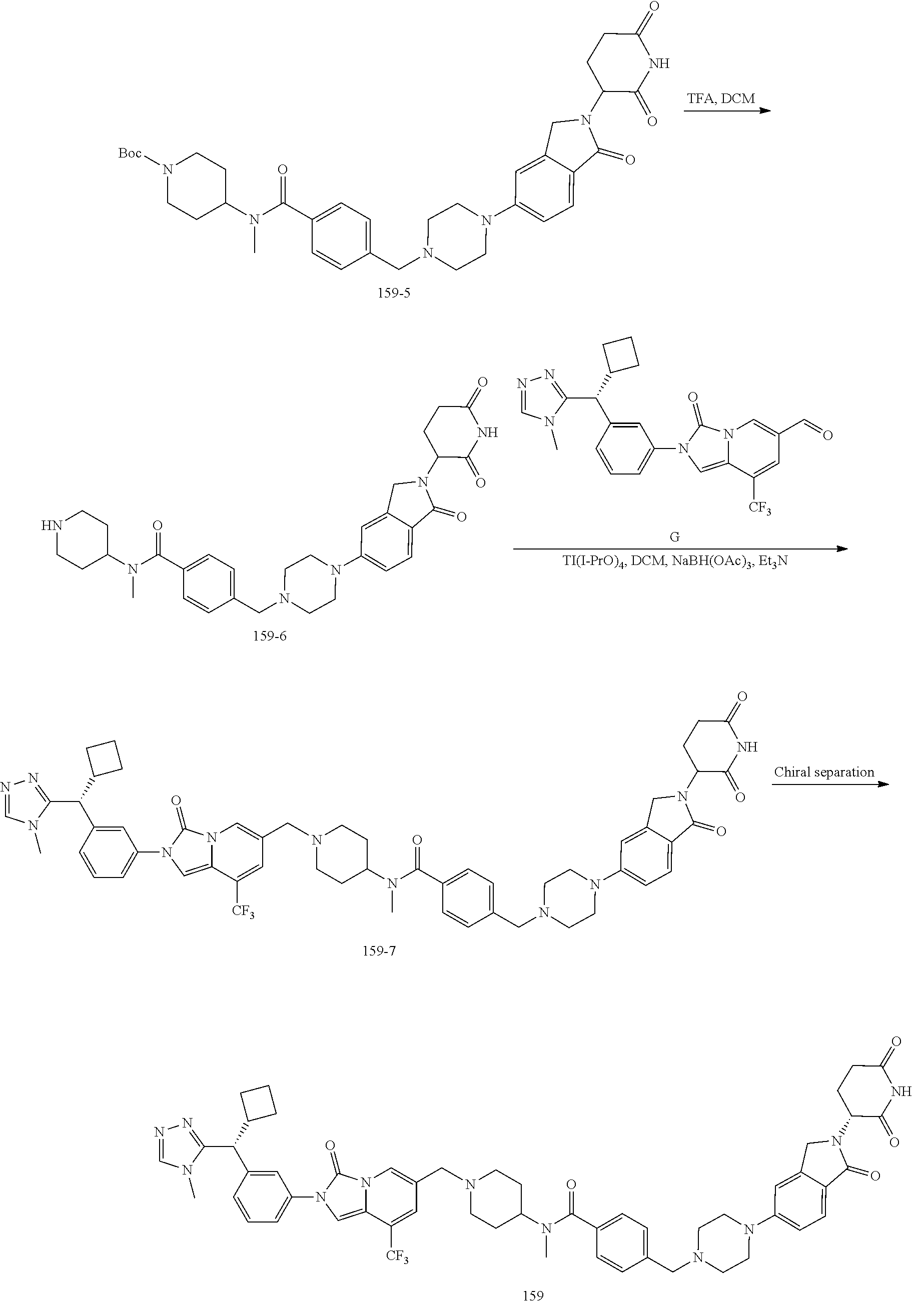

### Synthesis of Compound 159-1.

To a stirred solution of 3-(5-bromo-1-oxo-3H-isoindol-2-yl)piperidine-2,6-dione (20 g, 61.892 mmol, 1 equiv) and benzyl piperazine-1-carboxylate (16.36 g, 74.270 mmol, 1.2 equiv) in 1,4-dioxane (200 mL) were added Cs₂CO₃ (60.50 g, 185.676 mmol, 3 equiv) and Pd-PEPPSI-IPentCl 2-methylpyridine (o-picoline) (5.21 g, 6.189 mmol, 0.1 equiv) at room temperature under nitrogen atmosphere. The resulting mixture was stirred for overnight at 100° C. under nitrogen atmosphere. The reaction was quenched by the addition of 10% AcOH(aq) (500 mL) at room temperature. The aqueous layer was extracted with MeOH/CH₂Cl₂ (1:10) (3×300 mL). The resulting mixture was concentrated under reduced pressure. The residue was purified by silica gel column chromatography, eluted with CH₂Cl₂/MeOH (30:1) to afford Compound 159-1 (13 g, 45.41%) as a off-white solid.

### Synthesis of Compound 159-2.

To a solution of Compound 159-1 (13 g, 28.108 mmol, 1 equiv) in 390 mL MeOH was added Pd/C (10%, 3.9 g) under nitrogen atmosphere in a 1000 mL round-bottom flask. The mixture was hydrogenated at room temperature for 2 h under hydrogen atmosphere using a hydrogen balloon, filtered through a Celite pad and concentrated under reduced pressure. This resulted in Compound 159-2 (9 g, 97.51%) as a off-white solid.

### Synthesis of Compound 159-3.

To a stirred solution of Compound 159-2 (4 g, 12.181 mmol, 1 equiv) and DIEA (3.15 g, 24.362 mmol, 2 equiv) in DMF (40 mL) was added tert-butyl 4-(bromomethyl)benzoate (3.96 g, 14.617 mmol, 1.2 equiv) in portions at room temperature under nitrogen atmosphere. The resulting mixture was stirred for overnight at room temperature under nitrogen atmosphere. The reaction was quenched by the addition of sat. NH₄Cl (aq.) (200 mL) at room temperature. The resulting mixture was extracted with EtOAc (3×150 mL). The combined organic layers were washed with water (3×200 mL), dried over anhydrous Na₂SO₄. After filtration, the filtrate was concentrated under reduced pressure. The residue was purified by silica gel column chromatography, eluted with CH₂Cl₂/MeOH (30:1) to afford Compound 159-3 (5.3 g, 83.90%) as a off-white solid.

### Synthesis of Compound 159-4.

To a stirred solution of Compound 159-3 (6 g, 11.569 mmol, 1 equiv) in DCM (50 mL) was added TFA (10 mL) in portions at room temperature under nitrogen atmosphere. The resulting mixture was stirred for additional 3 h at room temperature. The resulting mixture was concentrated under reduced pressure. The residue was washed with MeCN (3×50 mL). This resulted in Compound 159-4 (8 g, crude) as a brown oil.

### Synthesis of Compound 159-5.

To a stirred solution of Compound 159-4 (4.2 g, 9.081 mmol, 1 equiv) and DIEA (3.52 g, 27.243 mmol, 3 equiv) in THF (42 mL) were added tert-butyl 4-(methylamino)piperidine-1-carboxylate (4.87 g, 22.703 mmol, 2.5 equiv) and T3P (17.34 g, 27.243 mmol, 3 equiv, 50% in EtOAc) in portions at room temperature under nitrogen atmosphere. The resulting mixture was stirred for additional 3 h at 60° C. The reaction was quenched by the addition of Water/Ice (100 mL) at room temperature. The aqueous layer was extracted with DCM/MeOH (5:1) (6×250 mL). The resulting mixture was concentrated under reduced pressure. The residue was purified by silica gel column chromatography, eluted with CH₂Cl₂/MeOH (20:1) to afford Compound 159-5 (4 g, 66.86%) as a brown oil.

### Synthesis of Compound 159-6.

To a stirred solution of Compound 159-5 (4 g, 6.072 mmol, 1 equiv) in DCM (12 mL) were added TFA (3 mL) in portions at room temperature under nitrogen atmosphere. The resulting mixture was stirred for additional 4 h at room temperature. The resulting mixture was concentrated under reduced pressure. This resulted in Compound 159-6 (4 g, crude) as a light yellow oil.

### Synthesis of Compound 159-7.

To a stirred solution of Compound 159-6 (1.2 g, 2.148 mmol, 1 equiv) and Intermediate G (0.98 g, 2.148 mmol, 1 equiv) in DCM (20 mL) were added Ti(Oi-Pr)₄ (3.05 g, 10.740 mmol, 5 equiv) and Et₃N (0.43 g, 4.296 mmol, 2 equiv). The resulting mixture was stirred for 2 h at room temperature under nitrogen atmosphere. To the above mixture was added NaBH(OAc)₃ (1.82 g, 8.592 mmol, 4 equiv). The resulting mixture was stirred for additional overnight at room temperature. The reaction was quenched by the addition of Water/HOAc=10/1 (150 mL) at room temperature. The resulting mixture was extracted with CH₂Cl₂/MeOH=10/1 (3×100 mL). The combined organic layers were concentrated under reduced pressure. The residue was purified by reversed-phase flash chromatography with the following conditions: column, C18 silica gel; mobile phase, MeCN in Water (0.1% FA), 10% to 80% gradient in 30 min; detector, UV 254 nm. The crude product (500 mg) was purified by Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 10% B to 26% B in 7 min, 26% B; Wave Length: 220 nm; RT1 (min): 6.37) to afford Compound 159-7 (150 mg, 7.00%) as a yellow solid.

### Synthesis of Compound 159.

Compound 159-7 (150 mg) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 90% B to 90% B in 14 min; Wave Length: 220/254 nm; RT1 (min): 7.68; RT2 (min): 11.39; The first peak was product) to afford Compound 159 (37.4 mg, 24.93%) as a yellow solid.

LCMS-Compound 159: (ES, m/z): [M+H]⁺ 987

NMR-Compound 159: (400 MHz, CD₃OD, δ ppm): δ1.63-1.89 (m, 10H), 2.00-2.06 (m, 1H), 2.10-2.31 (m, 2H), 2.31-2.39 (m, 1H), 2.53-2.69 (m, 5H), 2.72-2.84 (m, 4H), 3.02-3.08 (m, 1H), 3.16-3.22 (m, 3H), 3.27-3.38 (m, 5H), 3.43 (s, 3H), 3.60 (s, 2H), 4.16-4.19 (d, 1H), 4.28-4.31 (m, 2H), 4.98-5.02 (m, 1H), 6.98-7.04 (m, 4H), 7.15-7.17 (d, 1H), 7.26-7.31 (m, 2H), 7.36-7.41 (m, 3H), 7.52-7.54 (m, 4H), 8.25 (s, 1H).

### Example 158. Compound 160

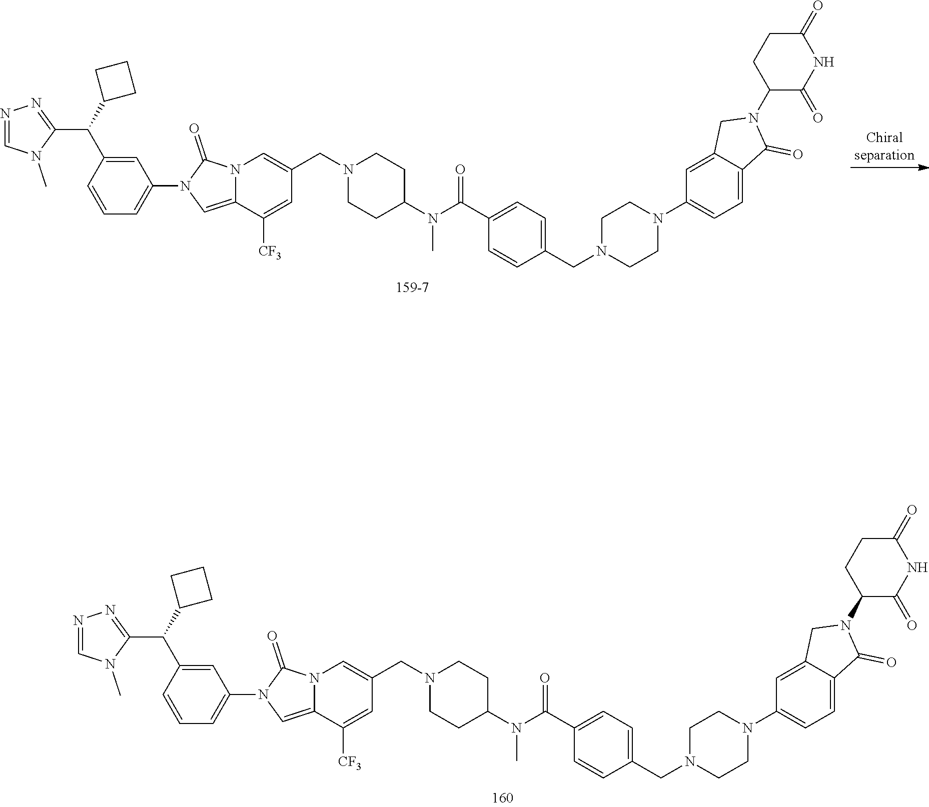 [SMILES: CN(C(=O)c1ccc(CN2CCN(c3ccc4c(c3)CN(C3CCC(=O)NC3=O)C4=O)CC2)cc1)C1CCN(Cc2cc(C(F)(F)F)c3cn(-c4cccc([C@H](c5nncn5C)C5CCC5)c4)c(=O)n3c2)CC1 ; CN(C(=O)c1ccc(CN2CCN(c3ccc4c(c3)CN([C@H]3CCC(=O)NC3=O)C4=O)CC2)cc1)C1CCN(Cc2cc(C(F)(F)F)c3cn(-c4cccc([C@H](c5nncn5C)C5CCC5)c4)c(=O)n3c2)CC1]

### Synthesis of Compound 160.

Compound 159-7 (150 mg, 0.150 mmol, 1 equiv)) was purified by Prep-HPLC with the following conditions (Column: CHIRALPAK IH, 2*25 cm, 5 μm; Mobile Phase A: MTBE (0.1% TEA)—HPLC, Mobile Phase B: DCM:ACN=1:1; Flow rate: 20 mL/min; Gradient: 90% B to 90% B in 14 min; Wave Length: 220/254 nm; RT1 (min): 7.68; RT2 (min): 11.39; The second peak was product) to afford Compound 160 (42.9 mg, 28.60%) as a yellow solid.

LCMS-Compound 160: (ES, m/z): [M/2+H]⁺ 499

NMR-Compound 160: (400 MHz, CD₃OD, δ ppm): δ1.63-1.89 (m, 10H), 2.00-2.06 (m, 1H), 2.10-2.16 (m, 1H), 2.18-2.24 (m, 2H), 2.28-2.31 (m, 1H), 2.56-2.62 (m, 4H), 2.63-2.66 (m, 1H), 2.70-2.91 (m, 5H), 3.02-3.08 (m, 1H), 3.16-3.22 (m, 3H), 3.27-3.38 (m, 5H), 3.44 (s, 3H), 3.62 (s, 2H), 4.16-4.19 (d, 1H), 4.28-4.31 (m, 2H), 4.98-5.02 (m, 1H), 6.98-7.04 (m, 4H), 7.15-7.17 (d, 1H), 7.26-7.31 (m, 2H), 7.36-7.41 (m, 3H), 7.52-7.54 (m, 4H), 8.25 (s, 1H).

### Example 159. Compound 161

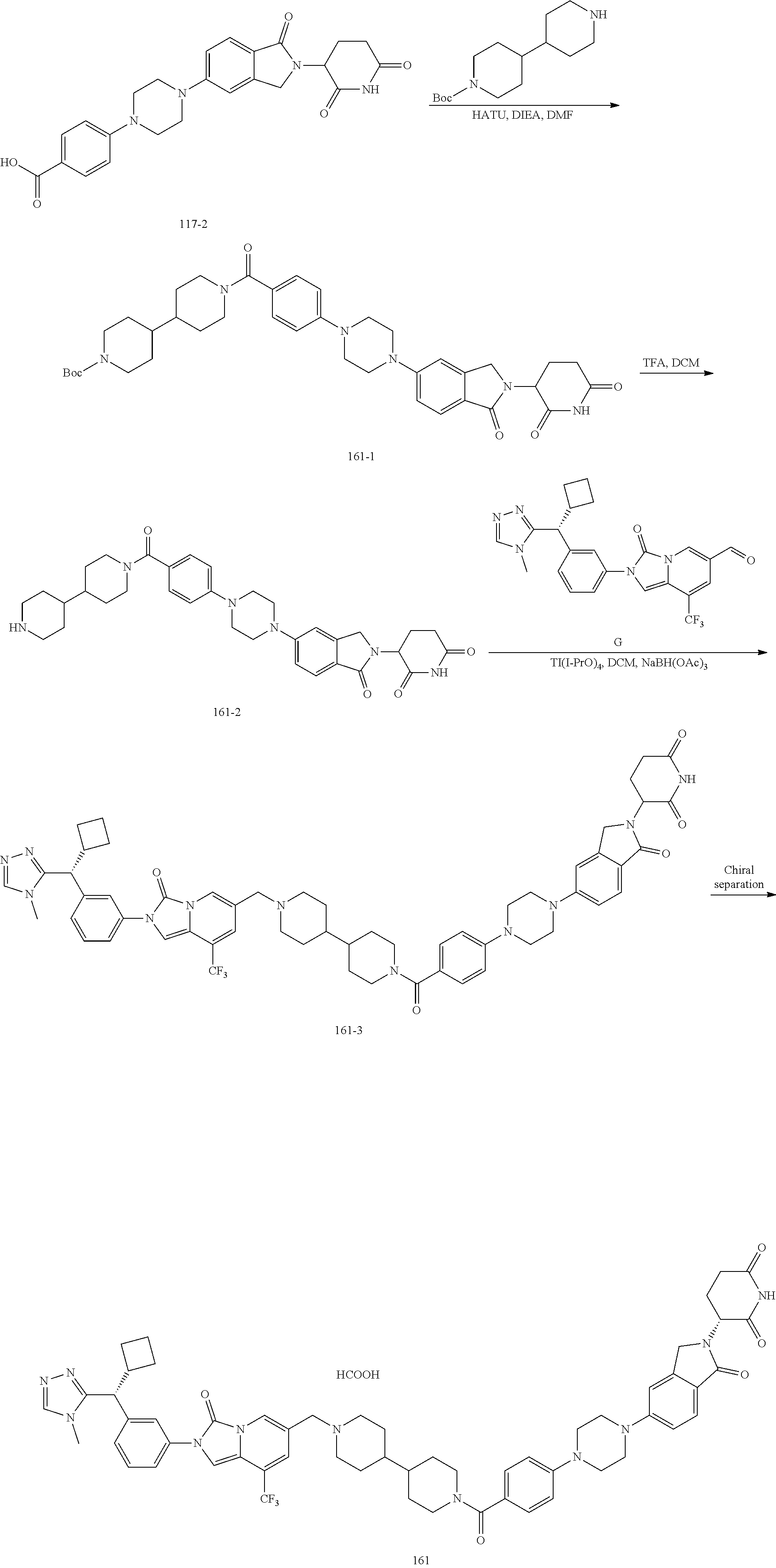

### Synthesis of Compound 161-1.

A solution of Compound 117-2 (3 g, 6.70 mmol, 1.0 equiv) in DMF (18 mL) was treated with HATU (5 g, 13.41 mmol, 2.0 equiv) and DIEA (1.7 g, 13.41 mmol, 2.0 equiv) for 1 h at room temperature under nitrogen atmosphere followed by the addition of tert-butyl [4,4′-bipiperidine]-1-carboxylate (1.8 g, 6.70 mmol, 1.0 equiv) at room temperature. The resulting mixture was stirred for 4 h at room temperature. The reaction was quenched by the addition of AcOH (10% in water) (60 mL) at room temperature. The residue was purified by silica gel column chromatography, eluted with CH₂Cl₂/MeOH (30:1) to afford Compound 162-1 (2.2 g, 47%) as a grey solid.

### Synthesis of Compound 161-2.

A solution of Compound 162-1 (1 g, 1.43 mmol, 1.0 equiv) in TFA (2 mL) and DCM (10 mL) was stirred for 4 h at room temperature. The resulting mixture was concentrated under vacuum. The residue was purified by Prep-TLC (CH₂Cl₂/MeOH=10:1) to afford Compound 162-2 (620 mg, 72%) as a grey solid.

### Synthesis of Compound 161-3.

To a stirred solution of Compound 162-2 (680 mg, 1.13 mmol, 1.0 equiv) and Intermediate G (620 mg, 1.36 mmol, 1.2 equiv) in DCM (7 mL) was added Ti(Oi-Pr)₄ (1.3 g, 4.54 mmol, 4.0 equiv) at room temperature. The resulting mixture was stirred for overnight at room temperature. To the above mixture was added NaBH(OAc)₃ (722 mg, 3.40 mmol, 3.0 equiv) at room temperature. The resulting mixture was stirred for additional 2 h at room temperature. The reaction was quenched by the addition of 10% AcOH (100 mL) at room temperature. The aqueous layer was extracted with EtOAc (2×60 mL). The aqueous phase was concentrated under reduced pressure. The residue was purified by reversed-phase flash chromatography with the following conditions: column, C18 silica gel; mobile phase, MeCN in water (0.1% FA), 10% to 50% gradient in 20 min; detector, UV 254 nm. The crude product (400 mg) was purified by Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 22% B to 33% B in 7 min, 33% B: Wave Length: 220; 254 nm; RT1 (min): 6.98; Number of Runs: 0) to afford Compound 162-3 (170 mg, 14%) as a yellow solid.

### Synthesis of Compound 161.

The Compound 161-3 (170 mg) was purified by Prep-CHIRAL-HPLC with the following conditions Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 60% B to 60% B in 11 min; Wave Length: 220/254 nm; RT1 (min): 5.36; RT2 (min): 7.96; Sample Solvent: DCM:ACN=1:1—HPLC; Injection Volume: 0.8 mL; Number Of Runs: 4. The first peak was the product. The residue was purified by reversed-phase flash chromatography with the following conditions: column, C18 silica gel; mobile phase, MeCN in water (0.1% FA), 10% to 50% gradient in 30 min; detector, UV 254 nm. This resulted in Compound 161 (54 mg, 34%) as a yellow solid.

LC-MS-Compound 161: (ES, m/z): [M-COOH]⁺ 1038

H-NMR-Compound 161: (400 MHz, DMSO-d6, δ ppm): 1.44-1.46 (m, 2H), 1.66-1.69 (m, 2H), 1.81-1.83 (m, 2H), 1.91-1.98 (m, 1H), 2.13-2.17 (m, 2H), 2.34-2.40 (m, 1H), 2.57-2.61 (m, 1H), 2.68-2.70 (m, 2H), 2.91-2.95 (m, 1H), 2.97 (s, 3H), 3.29-3.34 (m, 8H), 3.43-3.45 (m, 3H), 3.46-3.51 (m, 4H), 4.21-4.38 (m, 4H), 5.04-5.08 (m, 1H), 7.00-7.02 (m, 3H), 7.12-7.20 (m, 3H), 7.27-7.31 (m, 3H), 7.42-7.46 (m, 1H), 7.55-7.57 (d, 1H), 7.65-7.73 (m, 3H), 8.16 (s, 1H), 8.32 (s, 1H), 10.95 (s, 1H).

### Example 160. Compound 162

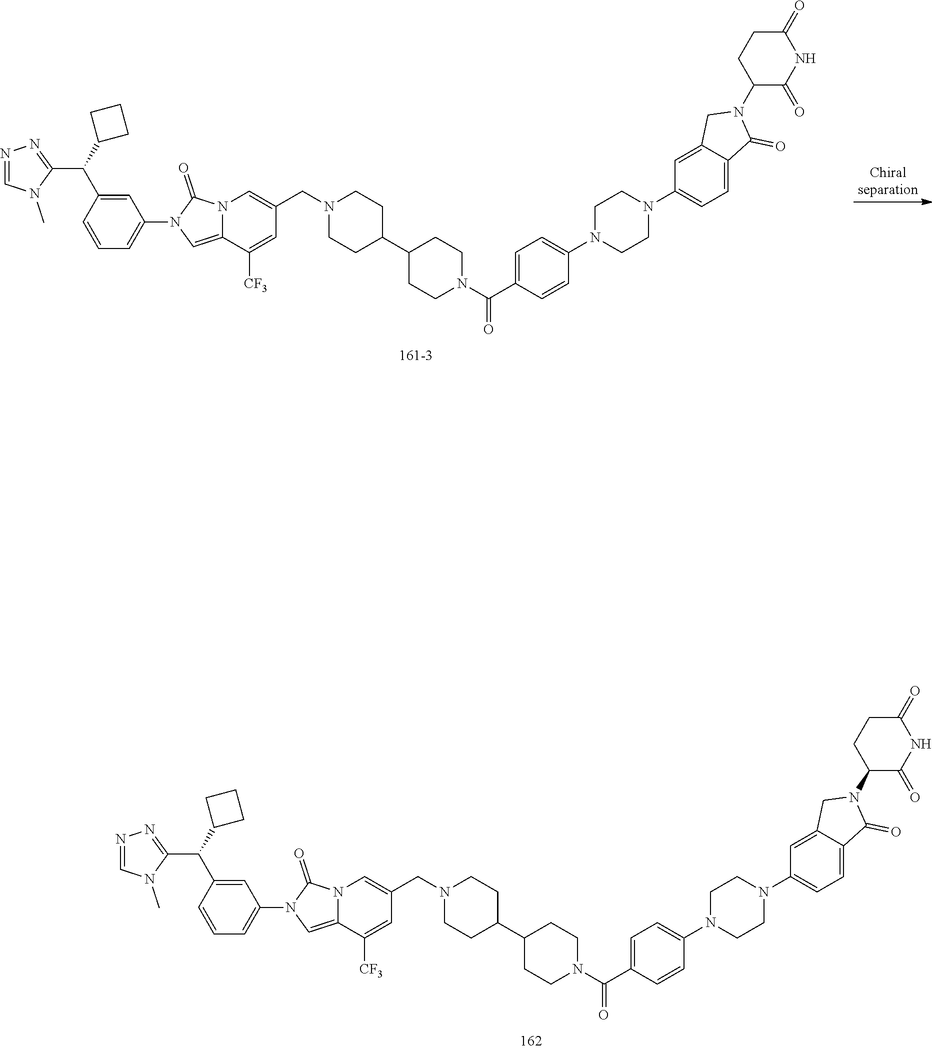 [SMILES: Cn1cnnc1[C@@H](c1cccc(-n2cc3c(C(F)(F)F)cc(CN4CCC(C5CCN(C(=O)c6ccc(N7CCN(c8ccc9c(c8)CN(C8CCC(=O)NC8=O)C9=O)CC7)cc6)CC5)CC4)cn3c2=O)c1)C1CCC1 ; Cn1cnnc1[C@@H](c1cccc(-n2cc3c(C(F)(F)F)cc(CN4CCC(C5CCN(C(=O)c6ccc(N7CCN(c8ccc9c(c8)CN([C@H]8CCC(=O)NC8=O)C9=O)CC7)cc6)CC5)CC4)cn3c2=O)c1)C1CCC1]

### Synthesis of Compound 162.

The Compound 161-3 (170 mg) was purified by Prep-CHIRAL-HPLC with the following conditions Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 60% B to 60% B in 11 min; Wave Length: 220/254 nm; RT1 (min): 5.36; RT2 (min): 7.96; Sample Solvent: DCM:ACN=1:1—HPLC; Injection Volume: 0.8 mL; Number Of Runs: 4. The second peak was the product. The residue was purified by reversed-phase flash chromatography with the following conditions: column, C18 silica gel; mobile phase, MeCN in water (0.1% FA), 10% to 50% gradient in 30 min; detector, UV 254 nm. This resulted in Compound 162 (37 mg, 24%) as a yellow solid.

LC-MS-Compound 162: (ES, m/z): [M-COOH]⁺ 1038

H-NMR-Compound 162: (400 MHz, DMSO-d6, δ ppm): 1.44-1.46 (m, 2H), 1.66-1.69 (m, 2H), 1.81-1.83 (m, 2H), 1.91-1.98 (m, 1H), 2.13-2.17 (m, 2H), 2.34-2.40 (m, 1H), 2.57-2.61 (m, 1H), 2.68-2.70 (m, 2H), 2.91-2.95 (m, 1H), 2.97 (s, 3H), 3.29-3.34 (m, 8H), 3.43-3.45 (m, 3H), 3.46-3.51 (m, 4H), 4.21-4.38 (m, 4H), 5.04-5.08 (m, 1H), 7.00-7.02 (m, 3H), 7.12-7.20 (m, 3H), 7.27-7.31 (m, 3H), 7.42-7.46 (m, 1H), 7.55-7.57 (d, 1H), 7.65-7.73 (m, 3H), 8.16 (s, 1H), 8.32 (s, 1H), 10.95 (s, 1H).

### Example 161. Compound 163

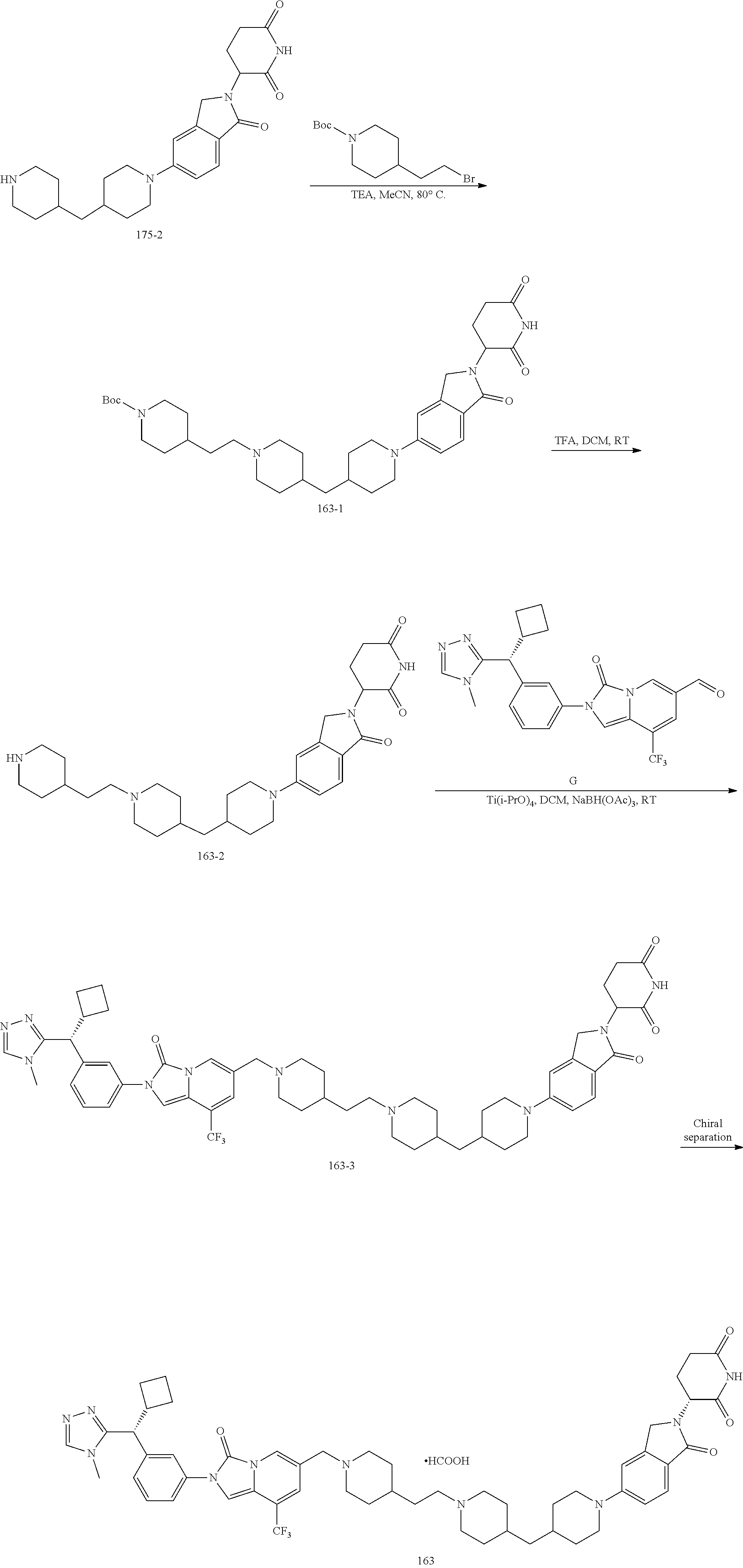

### Synthesis of Compound 163-1.

To a stirred solution of Compound 175-2 (2 g, 4.711 mmol, 1 equiv) and tert-butyl 4-(2-bromoethyl)piperidine-1-carboxylate (2.06 g, 7.067 mmol, 1.5 equiv) in MeCN (30 mL) was added TEA (1.43 g, 14.133 mmol, 3 equiv) at room temperature. The resulting mixture was stirred overnight at 80° C. The resulting mixture was concentrated under vacuum. The residue was purified by silica gel column chromatography, eluted with DCM (0.1% HOAc)/MeOH (20:1) to afford Compound 163-1 (2 g, 60.09%) as a light brown solid.

### Synthesis of Compound 163-2.

A solution of Compound 163-2 (2 g, 3.145 mmol, 1 equiv) and TFA (5 mL) in DCM (30 mL) was stirred for 3 h at room temperature. The resulting mixture was concentrated under vacuum. The residue was purified by reversed-phase flash chromatography with the following conditions: column, C18 silica gel; mobile phase, MeCN in water (0.1% FA), 0% to 40% gradient in 10 min; detector, UV 220 nm. This resulted in Compound 163-2 (1.5 g, 82.78%) as a light brown solid.

### Synthesis of Compound 163-3.

To a stirred solution of Compound 163-2 (1.5 g, 2.800 mmol, 1 equiv) and Intermediate G (1.28 g, 2.800 mmol, 1.00 equiv) in DCM (30 mL) was added Ti(Oi-Pr)₄ (2.39 g, 8.400 mmol, 3 equiv) at room temperature. The resulting mixture was stirred overnight at room temperature. To the above mixture was added NaBH(OAc)₃ (1.78 g, 8.400 mmol, 3 equiv) at room temperature. The resulting mixture was stirred for additional 3 h at room temperature. The reaction was quenched with 10% HOAc(aq.) (200 mL) at room temperature. The resulting mixture was filtered, the filter cake was washed with 10% HOAc(aq.) (2×10 mL). The aqueous layer was extracted with EtOAc (100 mL). The aqueous layer was concentrated under vacuum. The residue was purified by reversed-phase flash chromatography with the following conditions: column (C18 silica gel; mobile phase, MeCN in water (0.1% FA), 10% to 50% gradient in 20 min; detector, UV 254 nm) to afford the crude product. The crude product was purified by Prep-HPLC with the following conditions (Column: XBridge Prep OBD C18 Column, 30*150 mm, 5 μm; Mobile Phase A: Water (0.1% FA), Mobile Phase B: ACN; Flow rate: 60 mL/min; Gradient: 13% B to 24% B in 10 min, Wave Length: 254; 220 nm; RT1 (min): 9.05) to afford Compound 163-3 (330 mg, 11.72%) as a yellow solid.

### Synthesis of Compound 163.

The Compound 163-3 (330 mg) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 90% B to 90% B in 8.5 min; Wave Length: 220/254 nm; RT1 (min): 4.64; RT2 (min): 7.59; the first peak is product) to afford the crude product. The residue was purified by reverse flash chromatography with the following conditions (column, C18 silica gel; mobile phase, MeCN in water (0.1% FA), 0% to 40% gradient in 10 min; detector, UV 254 nm) to afford Compound 163 (120.1 mg, 33.71%) as a yellow solid.

LC-MS-Compound 163: (ES, m/z): [M−HCOOH+H]⁺ 975

H-NMR-Compound 163: (400 MHz, DMSO-d₆, δ ppm): 1.04-1.24 (m, 9H), 1.35-1.49 (m, 3H), 1.52-1.85 (m, 12H), 1.91-1.98 (m, 3H), 2.31-2.45 (m, 3H), 2.50-2.67 (m, 1H), 2.75-2.88 (m, 4H), 2.90-3.09 (m, 3H), 3.21-3.28 (m, 3H), 3.43 (s, 3H), 3.85-3.93 (m, 2H), 4.17-4.34 (m, 3H), 5.02-5.07 (d, 1H), 7.00-7.04 (m, 3H), 7.19-7.21 (d, 1H), 7.32 (s, 1H), 7.42-7.51 (m, 2H), 7.65-7.74 (m, 3H), 8.21 (s, 1H), 8.33 (s, 1H), 10.95 (s, 1H).

### Example 162. Compound 164

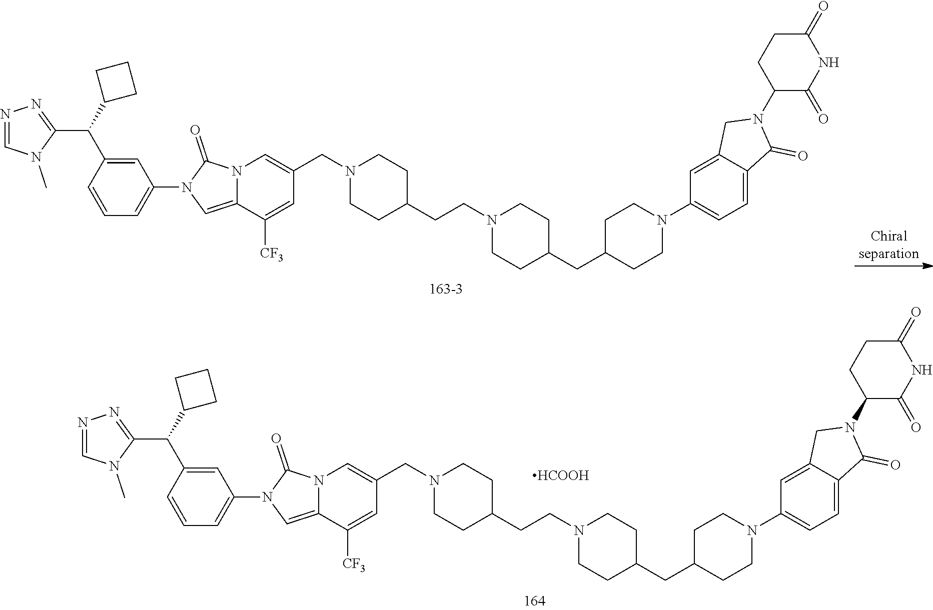 [SMILES: Cn1cnnc1[C@@H](c1cccc(-n2cc3c(C(F)(F)F)cc(CN4CCC(CCN5CCC(CC6CCN(c7ccc8c(c7)CN(C7CCC(=O)NC7=O)C8=O)CC6)CC5)CC4)cn3c2=O)c1)C1CCC1 ; Cn1cnnc1[C@@H](c1cccc(-n2cc3c(C(F)(F)F)cc(CN4CCC(CCN5CCC(CC6CCN(c7ccc8c(c7)CN([C@H]7CCC(=O)NC7=O)C8=O)CC6)CC5)CC4)cn3c2=O)c1)C1CCC1 ; "·HCOOH"]

### Synthesis of Compound 164.

The Compound 163-3 (330 mg) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 90% B to 90% B in 8.5 min; Wave Length: 220/254 nm; RT1 (min): 4.64; RT2 (min): 7.59; the second peak is product) to afford the crude product. The residue was purified by reverse flash chromatography with the following conditions (column, C18 silica gel; mobile phase, MeCN in water (0.1% FA), 0% to 40% gradient in 10 min; detector, UV 254 nm) to afford Compound 164 (123.0 mg, 35.34%) as a yellow solid.

LC-MS-Compound 164: (ES, m/z): [M−HCOOH+H]⁺ 975

H-NMR-Compound 164: (400 MHz, DMSO-d₆, δ ppm): 1.04-1.24 (m, 9H), 1.35-1.49 (m, 3H), 1.52-1.85 (m, 12H), 1.91-1.98 (m, 3H), 2.31-2.42 (m, 1H), 2.42-2.50 (m, 2H), 2.55-2.67 (m, 1H), 2.75-2.88 (m, 5H), 2.90-3.09 (m, 2H), 3.21-3.28 (m, 3H), 3.43 (s, 3H), 3.85-3.93 (m, 2H), 4.17-4.34 (m, 3H), 5.02-5.07 (d, 1H), 7.01-7.05 (m, 3H), 7.19-7.21 (d, 1H), 7.32 (s, 1H), 7.43-7.51 (m, 2H), 7.65-7.74 (m, 3H), 8.21 (s, 1H), 8.33 (s, 1H), 10.95 (s, 1H).

### Example 163. Compound 165

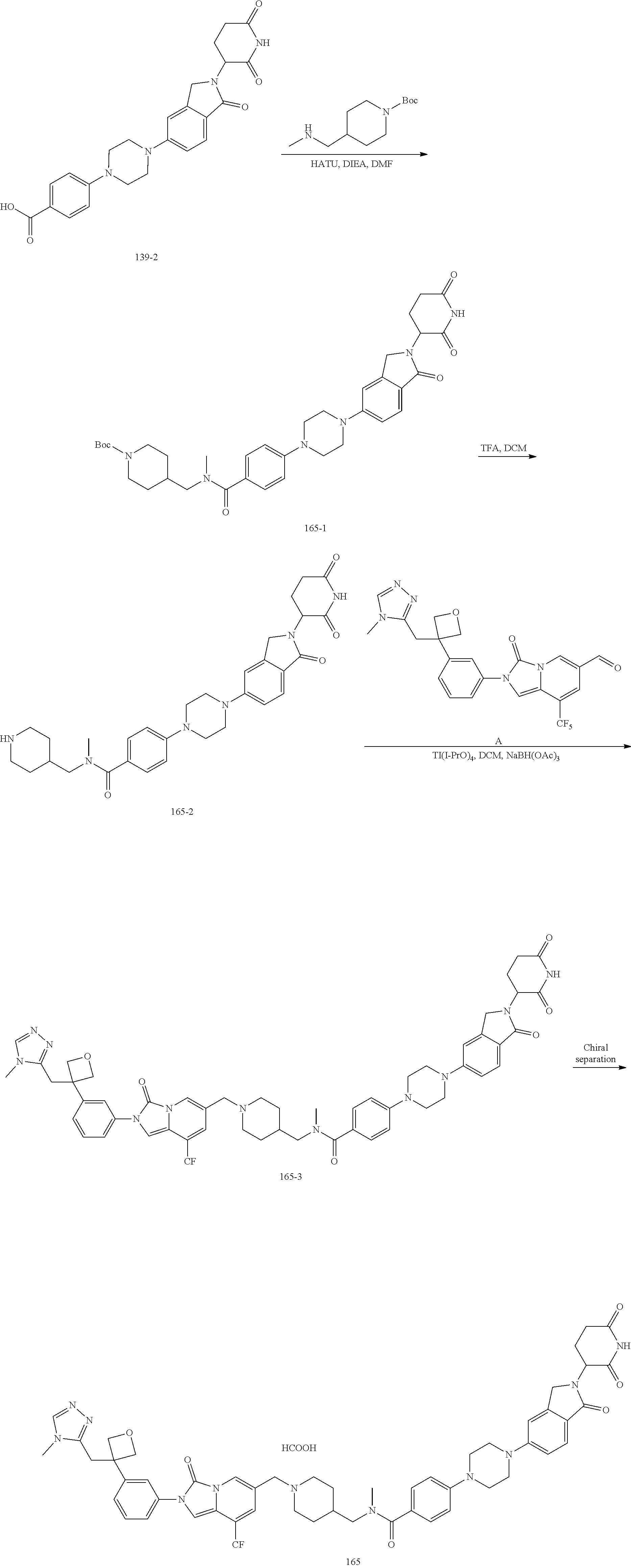

### Synthesis of Compound 165-1.

To a stirred solution of Compound 139-2 (6 g, 13.379 mmol, 1 equiv) and tert-butyl 4-[(methylamino)methyl]piperidine-1-carboxylate (3.36 g, 14.717 mmol, 1.1 equiv) in DMF (100 mL) was added HATU (6.61 g, 17.393 mmol, 1.3 equiv) and DIEA (3.46 g, 26.758 mmol, 2 equiv) at room temperature. The resulting mixture was stirred for 4 h at room temperature. The resulting mixture was diluted with water (300 mL). The precipitated solids were collected by filtration and washed with water (20 mL). This resulted in Compound 165-1 (6 g, 64.67%) as an off-white solid.

### Synthesis of Compound 165-2.

A solution of Compound 165-1 (6 g, 9.107 mmol, 1 equiv) in DCM (50 mL) and TFA (10 mL) was stirred for 3 h at room temperature. The resulting mixture was concentrated under vacuum. The product was precipitated by EtOAc (100 mL). This resulted in Compound 165-2 (5 g, 93.35%) as a brown solid.

### Synthesis of Compound 165-3.

To a stirred solution of Compound 165-2 (1 g, 1.884 mmol, 1 equiv) and Intermediate A (1.12 g, 2.449 mmol, 1.3 equiv) in DCM (20 mL) was added Ti(i-PrO)₄ (1.07 g, 3.768 mmol, 2 equiv) at room temperature. The resulting mixture was stirred for overnight at room temperature. To the above mixture was added NaBH(OAc)₃ (0.80 g, 3.768 mmol, 2 equiv) at room temperature. The resulting mixture was stirred for additional 4 h at room temperature. The reaction was quenched with 10% AcOH(aq.) (200 mL) at room temperature. The resulting mixture was filtered, the filter cake was washed with 10% AcOH (aq.) (2×20 mL). The aqueous layer was extracted with EtOAc (100 mL). The aqueous layer was concentrated under vacuum. The crude product was purified by Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 15% B to 35% B in 7.5 min, Wave Length: 220 nm; RT1 (min): 5.58) to afford Compound 165-3 (750 mg, 32.88%) as a yellow solid.

### Synthesis of Compound 165.

The Compound 165-3 (500 mg) was purified by Prep-Chiral-HPLC with the following conditions (Column: CHIRALPAK IH, 2*25 cm, 5 μm; Mobile Phase A: DCM:ACN=1:1 (0.2% TEA), Mobile Phase B: DCM:ACN=1:1 (0.2% TEA); Flow rate: 20 mL/min; Gradient: 50% B to 50% B in 45 min; Wave Length: 220/254 nm; RT1 (min): 10.87; RT2 (min): 33.78; the first peak is product) to afford the crude product. The crude product was purified by Prep-HPLC with the following conditions (Column: Sunfire prep C18 column, 30*150 mm, 5 μm; Mobile Phase A: Water (0.1% FA), Mobile Phase B: ACN; Flow rate: 60 mL/min; Gradient: 18% B to 28% B in 7 min, Wave Length: 254; 220 nm; RT1 (min): 6.22) to afford Compound 190 (157.5 mg, 31.5%) as a yellow solid.

LC-MS-Compound 165: (ES, m/z): [M−HCOOH+H]⁺ 1000

H-NMR-Compound 165: (400 MHz, DMSO-d₆, δ ppm): 0.61-1.43 (m, 2H), 1.45-1.83 (m, 3H), 1.92-2.08 (m, 3H), 2.26-2.38 (m, 1H), 2.61-2.70 (m, 1H), 2.82-2.85 (m, 2H), 2.92-2.97 (m, 7H), 3.19-3.25 (m, 6H), 3.39-3.46 (m, 5H), 3.51-3.52 (m, 3H), 4.20-4.25 (d, 1H) 4.33-4.37 (d, 1H), 4.90-4.95 (m, 4H), 5.06-5.12 (d, 1H), 6.88-6.90 (d, 1H), 6.99-7.01 (m, 3H), 7.12-7.14 (m, 2H), 7.27-7.30 (m, 3H), 7.37-7.41 (m, 2H), 7.54-7.56 (d, 1H), 7.73-7.76 (d, 1H), 7.79-7.83 (d, 1H) 8.14 (s, 1H), 8.19 (s, 1H), 10.95 (s, 1H).

### Example 164. Compound 166

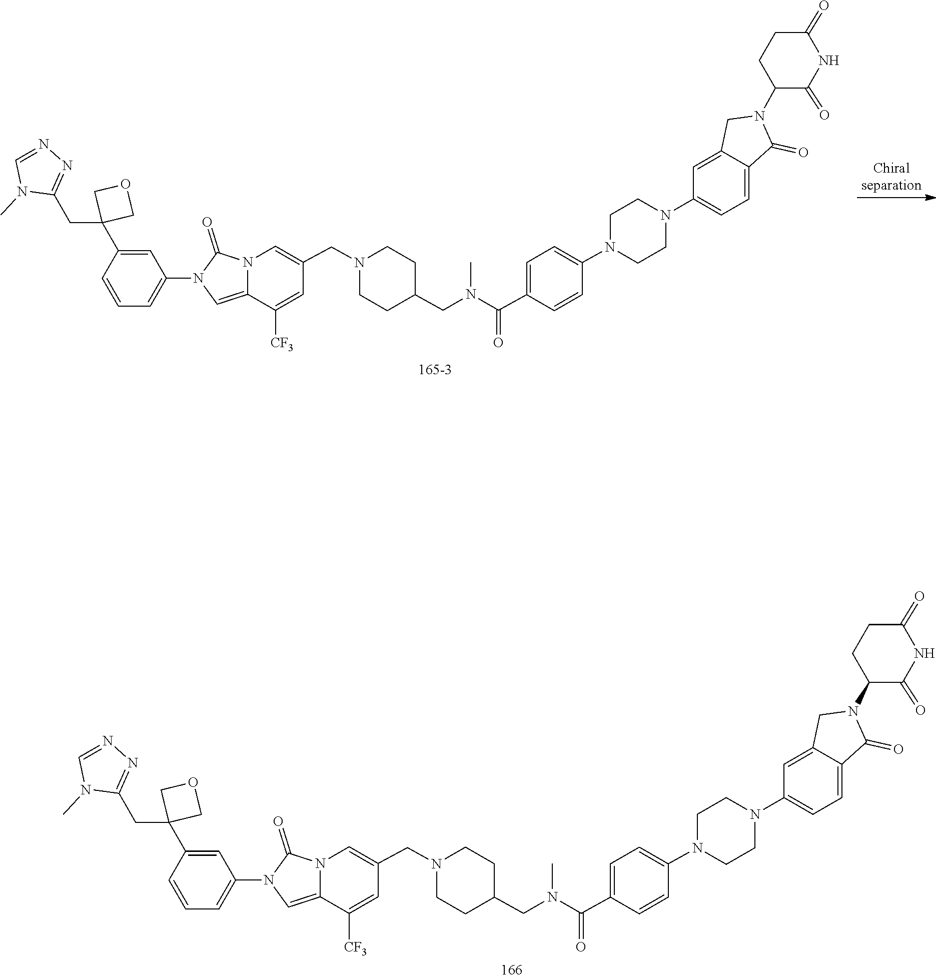 [SMILES: CN(CC1CCN(Cc2cc(C(F)(F)F)c3cn(-c4cccc(C5(Cc6nncn6C)COC5)c4)c(=O)n3c2)CC1)C(=O)c1ccc(N2CCN(c3ccc4c(c3)CN(C3CCC(=O)NC3=O)C4=O)CC2)cc1 ; CN(CC1CCN(Cc2cc(C(F)(F)F)c3cn(-c4cccc(C5(Cc6nncn6C)COC5)c4)c(=O)n3c2)CC1)C(=O)c1ccc(N2CCN(c3ccc4c(c3)CN([C@H]3CCC(=O)NC3=O)C4=O)CC2)cc1]

### Synthesis of Compound 166.

The Compound 165-3 (500 mg) was purified by Prep-Chiral-HPLC with the following conditions (Column: CHIRALPAK IH, 2*25 cm, 5 μm; Mobile Phase A: DCM:ACN=1:1 (0.2% TEA), Mobile Phase B: DCM:ACN=1:1 (0.2% TEA); Flow rate: 20 mL/min; Gradient: 50% B to 50% B in 45 min; Wave Length: 220/254 nm; RT1 (min): 10.87; RT2 (min): 33.78; the second peak is product) to afford the crude product. The crude product was purified by Prep-HPLC with the following conditions (Column: Sunfire prep C18 column, 30*150 mm, 5 μm; Mobile Phase A: Water (0.1% FA), Mobile Phase B: ACN; Flow rate: 60 mL/min; Gradient: 18% B to 28% B in 7 min, Wave Length: 254; 220 nm; RT1 (min): 6.17) to afford Compound 190 (148.3 mg, 29.66%) as a yellow solid

LC-MS-Compound 166: (ES, m/z): [M−HCOOH+H]⁺ 1000

H-NMR-Compound 166: (400 MHz, DMSO-d₆, δ ppm): 0.61-1.43 (m, 2H), 1.45-1.83 (m, 3H), 1.92-2.08 (m, 3H), 2.26-2.38 (m, 1H), 2.61-2.70 (m, 1H), 2.82-2.97 (m, 9H), 3.21-3.28 (m, 3H), 3.39-3.46 (m, 5H), 3.51-3.52 (m, 4H), 3.53 (s, 2H), 4.20-4.25 (d, 1H) 4.33-4.37 (d, 1H), 4.90-4.95 (m, 4H), 5.06-5.12 (d, 1H), 6.88-6.90 (d, 1H), 6.99-7.01 (m, 3H), 7.12-7.14 (m, 2H), 7.27-7.30 (m, 3H), 7.37-7.41 (m, 2H), 7.54-7.56 (d, 1H), 7.73-7.76 (d, 1H), 7.79-7.83 (d, 1H) 8.14 (s, 1H), 8.19 (s, 1H), 10.95 (s, 1H).

### Example 165. Compound 167

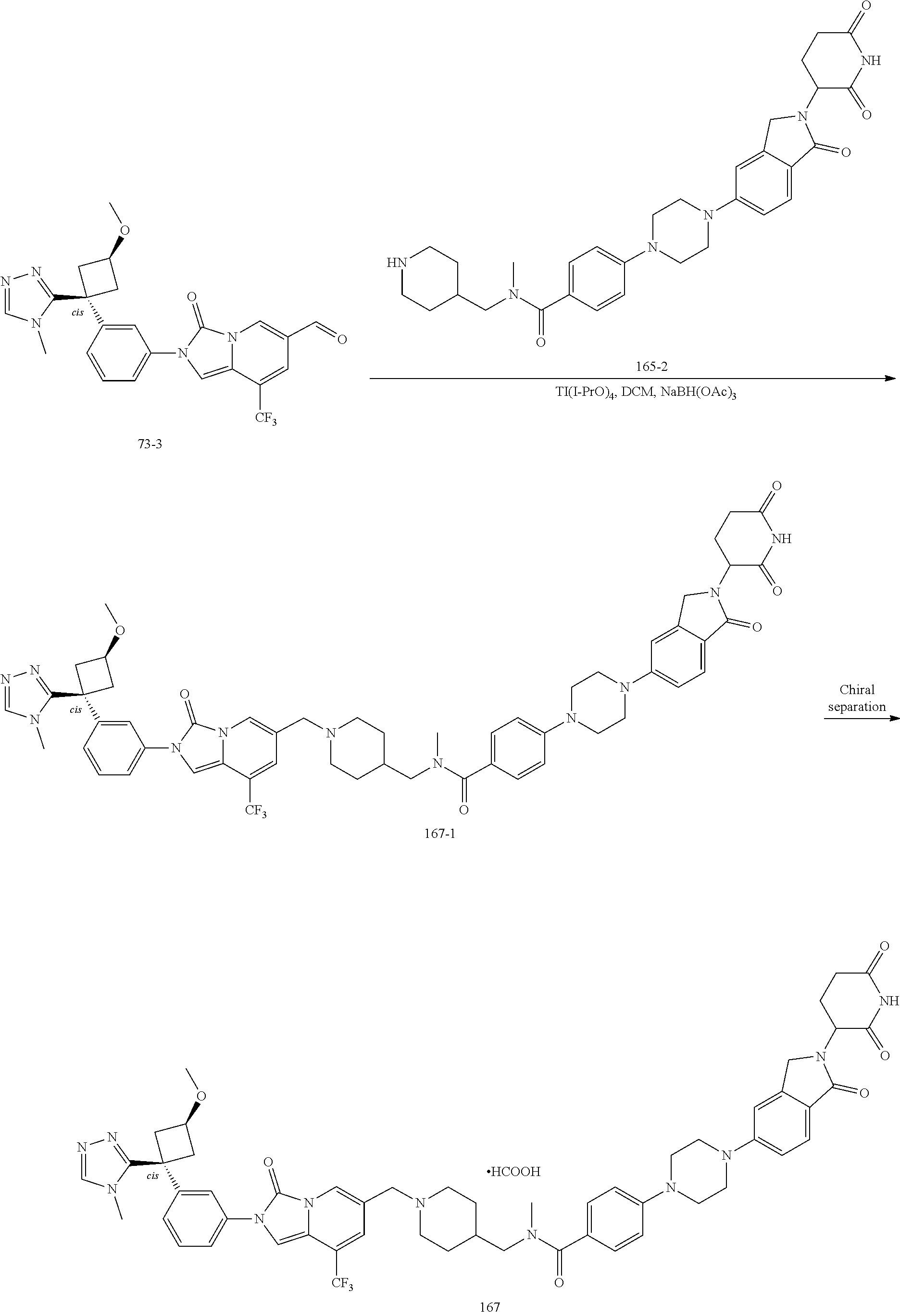 [SMILES: CO[C@H]1C[C@@](c2cccc(-n3cc4c(C(F)(F)F)cc(C=O)cn4c3=O)c2)(c2nncn2C)C1 ; CN(CC1CCNCC1)C(=O)c1ccc(N2CCN(c3ccc4c(c3)CN(C3CCC(=O)NC3=O)C4=O)CC2)cc1 ; CO[C@H]1C[C@@](c2cccc(-n3cc4c(C(F)(F)F)cc(CN5CCC(CN(C)C(=O)c6ccc(N7CCN(c8ccc9c(c8)CN(C8CCC(=O)NC8=O)C9=O)CC7)cc6)CC5)cn4c3=O)c2)(c2nncn2C)C1 ; CO[C@H]1C[C@@](c2cccc(-n3cc4c(C(F)(F)F)cc(CN5CCC(CN(C)C(=O)c6ccc(N7CCN(c8ccc9c(c8)CN(C8CCC(=O)NC8=O)C9=O)CC7)cc6)CC5)cn4c3=O)c2)(c2nncn2C)C1 ; "·HCOOH"]

### Synthesis of Compound 167-1.

To a stirred solution of Compound 73-3 (600 mg, 1.273 mmol, 1 equiv) and Compound 165-2 (711.03 mg, 1.273 mmol, 1 equiv) in DCM (20 mL) were added Ti(Oi-Pr)₄ (1085.17 mg, 3.819 mmol, 3 equiv) at room temperature. The resulting mixture was stirred overnight at room temperature. To the above mixture was added NaBH(OAc₃ (1078.94 mg, 5.092 mmol, 4 equiv) at room temperature. The resulting mixture was stirred for additional 4 h at room temperature. The reaction was quenched with HOAc at room temperature. The resulting mixture was concentrated under vacuum. The residue was purified by reverse flash chromatography with the following conditions: column, C18 silica gel; mobile phase, MeCN in water (0.1% FA), 0% to 60% gradient in 30 min; detector, UV 254 nm. This resulted in Compound 167-1 (480 ng, 34.96%) as a yellow solid.

### Synthesis of Compound 167.

The Compound 167-1 (480 mg) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 90% B to 90% B in 30 min; Wave Length: 220/254 nm; RT1 (min): 9.56; RT2 (min): 25.49; the first peak is product) to afford the crude product. The crude product was purified by reverse flash chromatography with the following conditions (column, C18 silica gel; mobile phase, MeCN in water (0.1% FA), 0% to 40% gradient in 10 min; detector, UV 254 nm) to afford Compound 167 (174.7 mg, 33.49%) as a yellow solid.

LC-MS-Compound 167: (ES, m/z): [M−HCOOH+H]⁺ 1014

H-NMR-Compound 167: (400 MHz, DMSO-d₆, δ ppm): 0.80-1.29 (m, 2H), 1.41-1.79 (m, 3H), 1.95-2.15 (m, 3H), 2.31-2.45 (m, 1H), 2.52-2.62 (m, 1H), 2.78-2.90 (m, 4H), 2.90-3.01 (m, 4H), 3.09-3.12 (m, 1H), 3.17 (s, 3H), 3.22-3.24 (m, 4H), 3.30-3.40 (m, 4H), 3.40-3.46 (m, 4H), 3.46-3.51 (m, 4H), 4.06-4.08 (m, 1H), 4.21-4.26 (m, 1H), 4.34-4.38 (m, 1H), 5.06-5.08 (d, 1H), 6.95-7.02 (m, 3H), 7.13-7.15 (m, 2H), 7.25-7.30 (m, 3H), 7.40 (s, 1H), 7.49-7.53 (m, 1H), 7.53-7.57 (d, 1H), 7.66-7.68 (m, 2H), 7.78 (s, 1H), 8.14 (s, 1H), 8.31 (s, 1H), 10.95 (s, 1H).

### Example 166. Compound 168

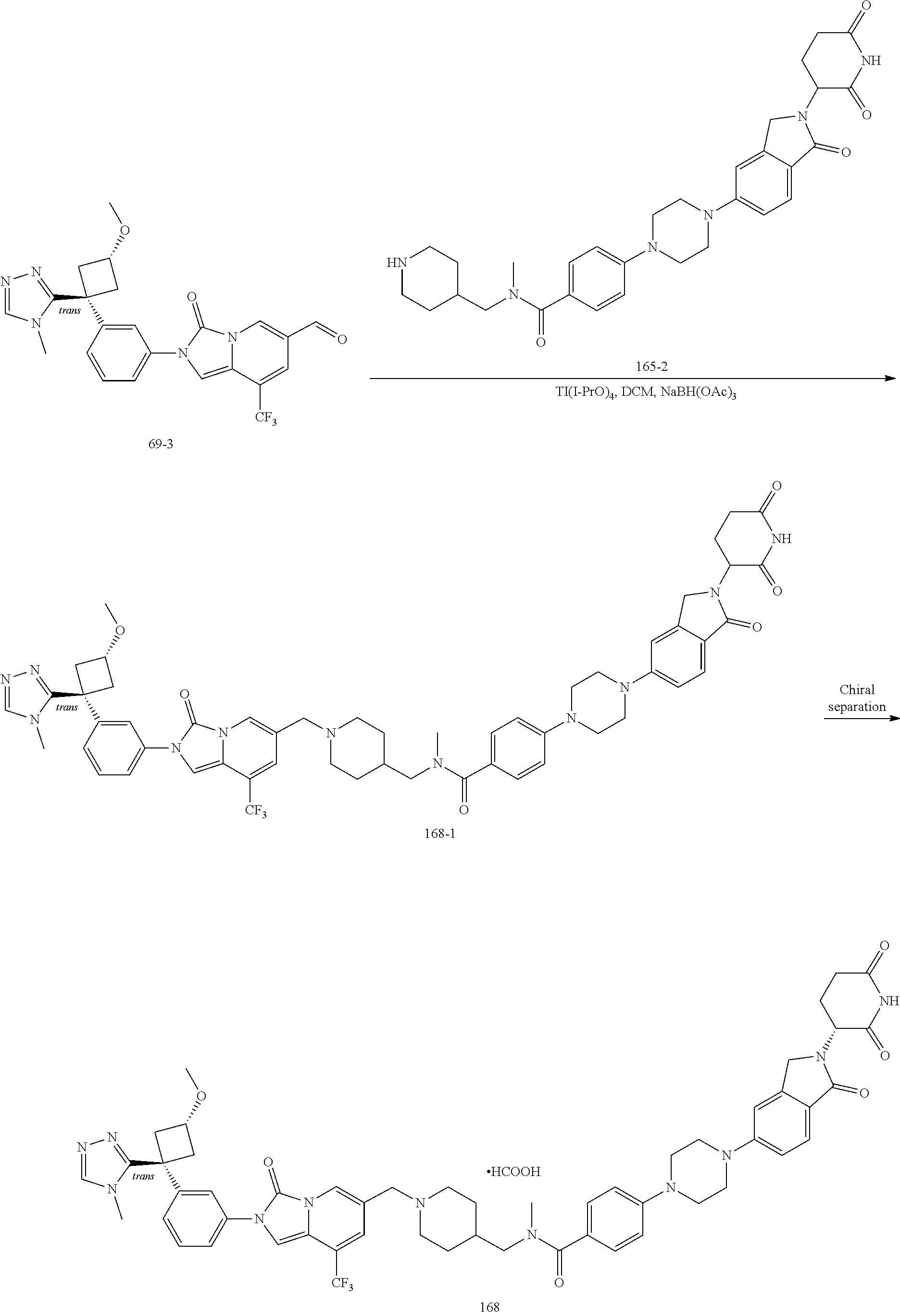 [SMILES: CO[C@H]1C[C@](c2cccc(-n3cc4c(C(F)(F)F)cc(C=O)cn4c3=O)c2)(c2nncn2C)C1 ; CN(CC1CCNCC1)C(=O)c1ccc(N2CCN(c3ccc4c(c3)CN(C3CCC(=O)NC3=O)C4=O)CC2)cc1 ; CO[C@H]1C[C@](c2cccc(-n3cc4c(C(F)(F)F)cc(CN5CCC(CN(C)C(=O)c6ccc(N7CCN(c8ccc9c(c8)CN(C8CCC(=O)NC8=O)C9=O)CC7)cc6)CC5)cn4c3=O)c2)(c2nncn2C)C1 ; CO[C@H]1C[C@](c2cccc(-n3cc4c(C(F)(F)F)cc(CN5CCC(CN(C)C(=O)c6ccc(N7CCN(c8ccc9c(c8)CN([C@@H]8CCC(=O)NC8=O)C9=O)CC7)cc6)CC5)cn4c3=O)c2)(c2nncn2C)C1 ; "·HCOOH"]

### Synthesis of Compound 168-1.

To a stirred solution of Compound 69-3 (300 mg, 0.636 mmol, 1 equiv) and Compound 165-2 (355.52 mg, 0.636 mmol, 1 equiv) in DCM (15 mL) were added Ti(Oi-Pr)₄ (542.59 mg, 1.908 mmol, 3 equiv) at room temperature. The resulting mixture was stirred for overnight at room temperature. To the above mixture was added NaBH(OAc)₃ (539.47 mg, 2.544 mmol, 4 equiv) at room temperature. The resulting mixture was stirred for additional 4 h at room temperature. The reaction was quenched with HOAc at room temperature. The resulting mixture was concentrated under vacuum. The residue was purified by reverse flash chromatography with the following conditions: column, C18 silica gel; mobile phase, MeCN in water (0.1% FA), 0% to 60% gradient in 20 min; detector, UV 254 nm. This resulted in Compound 168-1 (180 mg, 25.94%) as a yellow solid.

### Synthesis of Compound 168.

The Compound 168-1 (180 mg, 0.177 mmol, 1 equiv) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 90% B to 90% B in 30 min; Wave Length: 220/254 nm; RT1 (min): 7.76; RT2 (min): 20.41; the first peak is product) to afford the crude product. The crude product was purified by reverse flash chromatography with the following conditions (column, C18 silica gel; mobile phase, MeCN in water (0.1% FA), 0% to 40% gradient in 10 min; detector, UV 254 nm) to afford Compound 168 (57.3 mg, 29.51%) as a yellow solid.

LC-MS-Compound 168: (ES, m/z): [M−HCOOH+H]⁺ 1014

H-NMR-Compound 168: (400 MHz, DMSO-d₆, δ ppm): 0.80-1.29 (m, 2H), 1.41-1.79 (m, 3H), 1.95-2.05 (m, 3H), 2.31-2.45 (m, 1H), 2.52-2.62 (m, 1H), 2.78-2.90 (m, 2H), 3.18 (s, 3H), 3.24 (s, 3H), 3.30-3.34 (m, 6H), 3.38-3.40 (m, 2H), 3.40-3.46 (m, 4H), 3.46-3.51 (m, 4H), 3.84-3.88 (m, 1H), 4.21-4.26 (m, 1H), 4.34-4.38 (m, 1H), 5.04-5.08 (d, 1H), 6.95-7.03 (m, 3H), 7.07-7.09 (d, 1H), 7.13-7.15 (m, 2H), 7.28-7.30 (m, 2H), 7.40 (s, 1H), 7.46-7.50 (m, 1H), 7.55-7.57 (d, 1H), 7.65-7.70 (m, 3H), 8.16 (s, 1H), 8.39 (s, 1H), 10.95 (s, 1H).

### Example 167. Compound 169

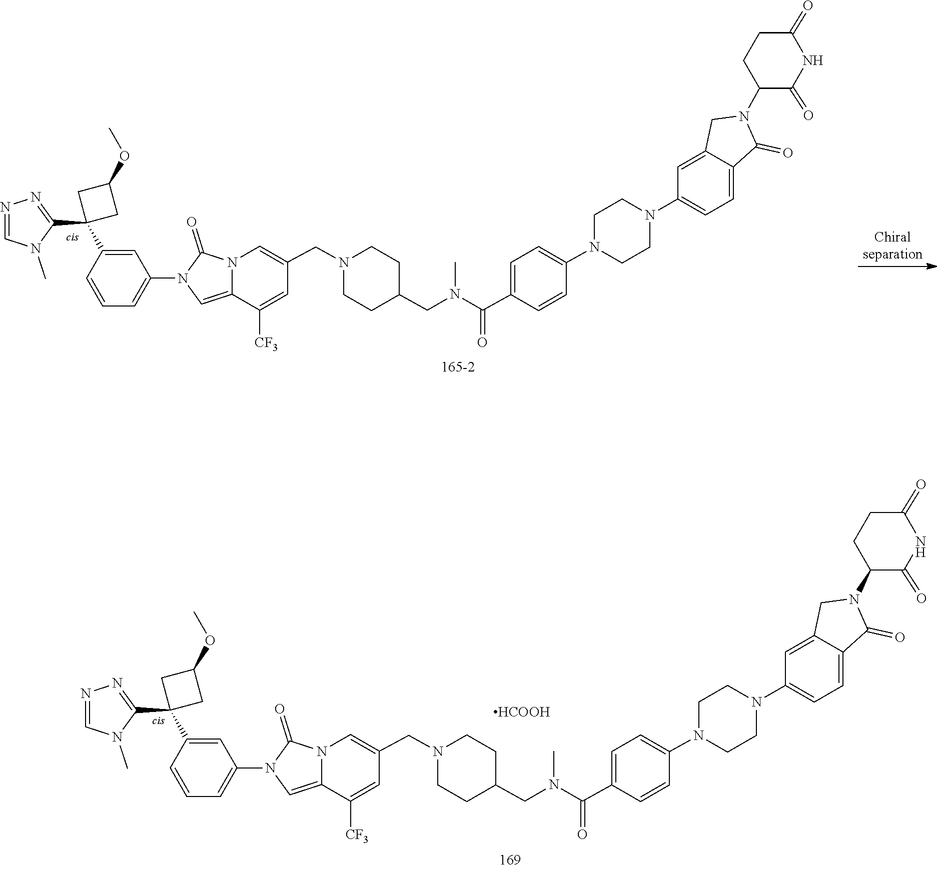 [SMILES: CO[C@H]1C[C@@](c2cccc(-n3cc4c(C(F)(F)F)cc(CN5CCC(CN(C)C(=O)c6ccc(N7CCN(c8ccc9c(c8)CN(C8CCC(=O)NC8=O)C9=O)CC7)cc6)CC5)cn4c3=O)c2)(c2nncn2C)C1 ; CO[C@H]1C[C@@](c2cccc(-n3cc4c(C(F)(F)F)cc(CN5CCC(CN(C)C(=O)c6ccc(N7CCN(c8ccc9c(c8)CN([C@H]8CCC(=O)NC8=O)C9=O)CC7)cc6)CC5)cn4c3=O)c2)(c2nncn2C)C1 ; "·HCOOH"]

### Synthesis of Compound 169.

The Compound 167-1 (480 mg, 0.473 mmol, 1 equiv) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 90% B to 90% B in 30 min; Wave Length: 220/254 nm; RT1 (min): 9.56; RT2 (min): 25.49; the second peak is product) to afford the crude product. The crude product was purified by reverse flash chromatography with the following conditions (column, C18 silica gel; mobile phase, MeCN in water (0.1% FA), 0% to 40% gradient in 10 min; detector, UV 254 nm) to afford Compound 169 (174.7 mg, 33.49%) as a yellow solid.

LC-MS-Compound 169: (ES, m/z): [M−HCOOH+H]⁺ 1014

H-NMR-Compound 169: (400 MHz, DMSO-d₆, δ ppm): 0.80-1.29 (m, 2H), 1.41-1.79 (m, 3H), 1.95-2.15 (m, 3H), 2.31-2.45 (m, 1H), 2.52-2.62 (m, 1H), 2.78-2.90 (m, 4H), 2.90-3.01 (m, 4H), 3.09-3.12 (m, 2H), 3.17 (s, 3H), 3.22-3.24 (m, 3H), 3.30-3.40 (m, 2H), 3.40-3.46 (m, 5H), 3.46-3.51 (m, 5H), 4.06-4.08 (m, 1H), 4.21-4.26 (m, 1H), 4.34-4.38 (m, 1H), 5.06-5.08 (d, 1H), 6.95-7.02 (m, 3H), 7.13-7.15 (m, 2H), 7.25-7.30 (m, 3H), 7.40 (s, 1H), 7.49-7.53 (m, 1H), 7.53-7.57 (d, 1H), 7.66-7.68 (m, 2H), 7.78 (s, 1H), 8.14 (s, 1H), 8.31 (s, 1H), 10.95 (s, 1H).

### Example 168. Compound 170

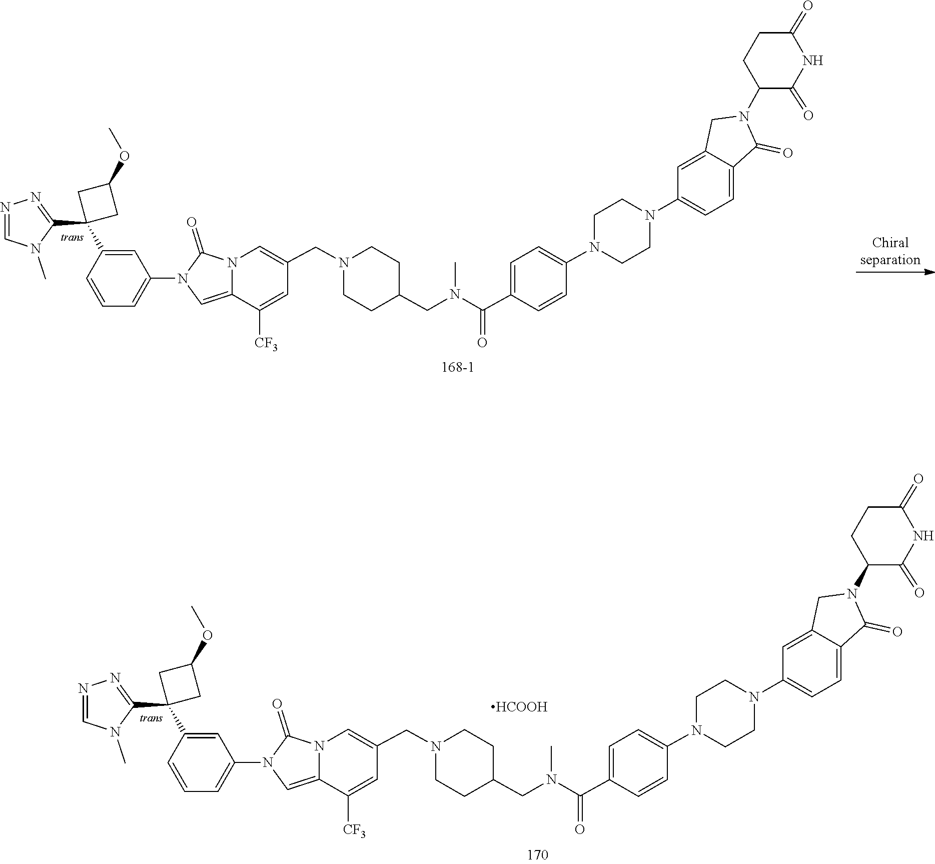 [SMILES: CO[C@H]1C[C@@](c2cccc(-n3cc4c(C(F)(F)F)cc(CN5CCC(CN(C)C(=O)c6ccc(N7CCN(c8ccc9c(c8)CN(C8CCC(=O)NC8=O)C9=O)CC7)cc6)CC5)cn4c3=O)c2)(c2nncn2C)C1 ; CO[C@H]1C[C@@](c2cccc(-n3cc4c(C(F)(F)F)cc(CN5CCC(CN(C)C(=O)c6ccc(N7CCN(c8ccc9c(c8)CN([C@H]8CCC(=O)NC8=O)C9=O)CC7)cc6)CC5)cn4c3=O)c2)(c2nncn2C)C1 ; "·HCOOH"]

### Synthesis of Compound 170.

The Compound 168-1 (180 mg) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 90% B to 90% B in 30 min; Wave Length: 220/254 nm; RT1 (min): 7.76; RT2 (min): 20.41; the first peak is product) to afford the crude product. The crude product was purified by reverse flash chromatography with the following conditions (column, C18 silica gel; mobile phase, MeCN in water (0.1% FA), 0% to 40% gradient in 10 min; detector, UV 254 nm) to afford Compound 170 (58.6 mg, 30.68%) as a yellow solid.

LC-MS-Compound 170: (ES, m/z): [M−HCOOH+H]⁺ 1014

H-NMR-Compound 170: (400 MHz, DMSO-d₆, δ ppm): 0.80-1.29 (m, 2H), 1.41-1.79 (m, 3H), 1.95-2.05 (m, 3H), 2.31-2.45 (m, 1H), 2.52-2.62 (m, 1H), 2.78-2.90 (m, 2H), 3.18 (s, 3H), 3.24-3.30 (m, 5H), 3.30-3.34 (m, 3H), 3.38-3.40 (m, 2H), 3.40-3.46 (m, 5H), 3.46-3.51 (m, 4H), 3.84-3.88 (m, 1H), 4.21-4.26 (m, 1H), 4.34-4.38 (m, 1H), 5.04-5.08 (d, 1H), 6.95-7.03 (m, 3H), 7.07-7.09 (d, 1H), 7.13-7.15 (m, 2H), 7.28-7.30 (m, 2H), 7.40 (s, 1H), 7.46-7.50 (m, 1H), 7.55-7.57 (d, 1H), 7.65-7.70 (m, 3H), 8.16 (s, 1H), 8.39 (s, 1H), 10.95 (s, 1H).

### Example 169. Compound 171

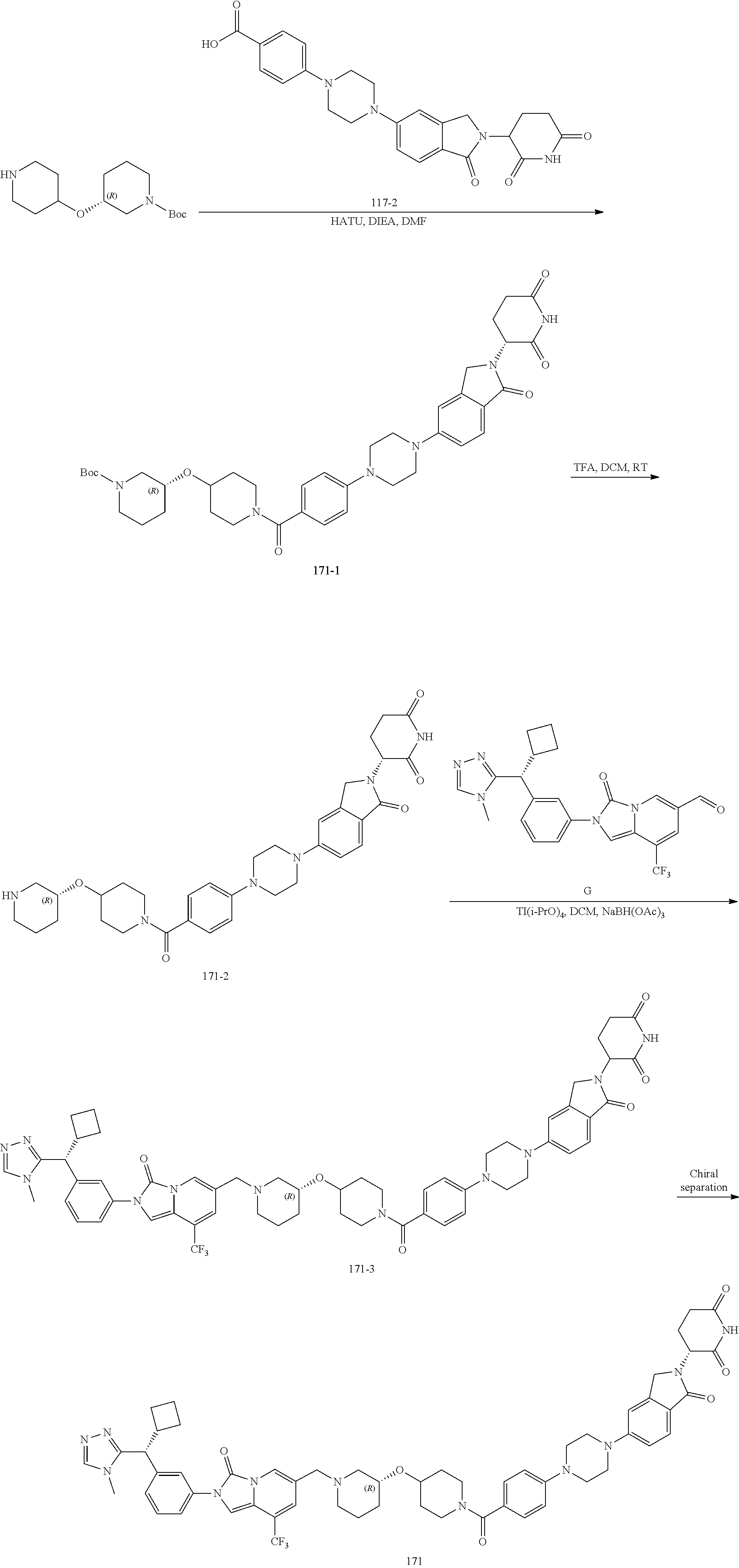

### Synthesis of Compound 171-1.

To a stirred solution of Compound 117-2 (6 g, 14.06 mmol, 1.0 equiv) and DIEA (4.5 g, 35.16 mmol, 2.5 equiv) in DMF (50 mL) was added HATU (13.4 g, 35.16 mmol, 2.5 equiv) at room temperature. The resulting mixture was stirred for 1 h at room temperature. To the above mixture was added tert-butyl (3R)-3-(piperidin-4-yloxy) piperidine-1-carboxylate (4 g, 14.06 mmol, 1 equiv) at room temperature. The resulting mixture was stirred for additional overnight at room temperature. The reaction was quenched by the addition of water (150 mL) at room temperature. The aqueous layer was extracted with EtOAc (3×50 mL). The resulting mixture was concentrated under reduced pressure. The residue was purified by silica gel column chromatography, eluted with CH₂Cl₂/MeOH (20:1) to afford Compound 171-1 (3 g, 21%) as a brown solid.

### Synthesis of Compound 171-2.

A solution of tert-butyl Compound 171-1 (3.5 g, 4.16 mmol, 1.0 equiv) and TFA (14 mL) in DCM (42 mL) was stirred for 1 h at room temperature under nitrogen atmosphere. The resulting mixture was concentrated under reduced pressure. The residue was purified by reversed-phase flash chromatography with the following conditions: column, C18 silica gel; mobile phase, MeCN in water (0.1% FA), 10% to 50% gradient in 30 min; detector, UV 254 nm. The resulting mixture was concentrated under vacuum. This resulted in Compound 171-2 (1.3 g, 51%) as a yellow solid.

### Synthesis of Compound 171-3.

To a stirred solution of Compound 171-2 (1.2 g, 2.08 mmol, 1.0 equiv) and Intermediate G (1.4 g, 3.13 mmol, 1.5 equiv) in DCM (13 mL) was added Ti(Oi-Pr)₄ (2.3 g, 8.32 mmol, 4.0 equiv) at room temperature. The resulting mixture was stirred for overnight at room temperature. To the above mixture was added NaBH(OAc)₃ (1.3 g, 6.24 mmol, 3.0 equiv) at room temperature. The resulting mixture was stirred for additional 2 h at room temperature. The reaction was quenched by the addition of 10% AcOH (150 mL) at room temperature. The aqueous layer was extracted with DCM:MeOH (5:1) (3×120 mL). The residue was purified by reversed-phase flash chromatography with the following conditions: column, C18 silica gel; mobile phase, MeCN in water (0.1% FA), 10% to 50% gradient in 30 min; detector, UV 254 nm. The crude product (550 mg) was purified by Prep-HPLC with the following conditions (Column: Sunfire prep C18 column, 30*150 mm, 5 μm; Mobile Phase A: Water (0.1% FA), Mobile Phase B: ACN; Flow rate: 60 mL/min; Gradient: 21% B to 32% B in 8 min, 32% B: Wave Length: 254; 220 nm; RT1 (min): 7.5; Number of Runs: 0) to afford Compound 171-3 (350 mg, 16%) as a yellow solid.

### Synthesis of Compound 171.

The Compound 171-3 (350 mg) was purified by Prep-CHIRAL-HPLC with the following conditions Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 90% B to 90% B in 20 min; Wave Length: 220/254 nm; RT1 (min): 9.82; RT2 (min): 17.01; Sample Solvent: DCM:ACN=1:1—HPLC; Injection Volume: 0.85 mL; Number of Runs: 5. The first peak was the product. This resulted in Compound 171 (103 mg, 29%) as a yellow solid.

LC-MS-Compound 171: (ES, m/z): [M+H]⁺ 1054

H-NMR-Compound 171: (400 MHz, DMSO-d6, δ ppm): 1.05-1.14 (m, 1H), 1.39-1.42 (m, 3H), 1.69-1.83 (m, 9H), 1.90-1.98 (m, 3H), 2.02-2.08 (m, 1H), 2.35-2.41 (m, 1H), 2.49-2.51 (m, 3H), 2.86-2.91 (m, 2H), 3.17-3.20 (m, 3H), 3.24-3.28 (m, 1H), 3.42-3.45 (m, 4H), 3.49-3.66 (m, 8H), 3.70-3.85 (m, 3H), 4.20-4.37 (m, 3H), 5.02-5.07 (m, 1H), 6.98-7.01 (m, 3H), 7.11-7.20 (m, 3H), 7.27-7.31 (m, 3H), 7.41-7.45 (m, 1H), 7.54-7.56 (m, 1H), 7.67-7.73 (m, 3H), 8.32 (s, 1H), 10.95 (s, 1H).

### Example 170. Compound 172

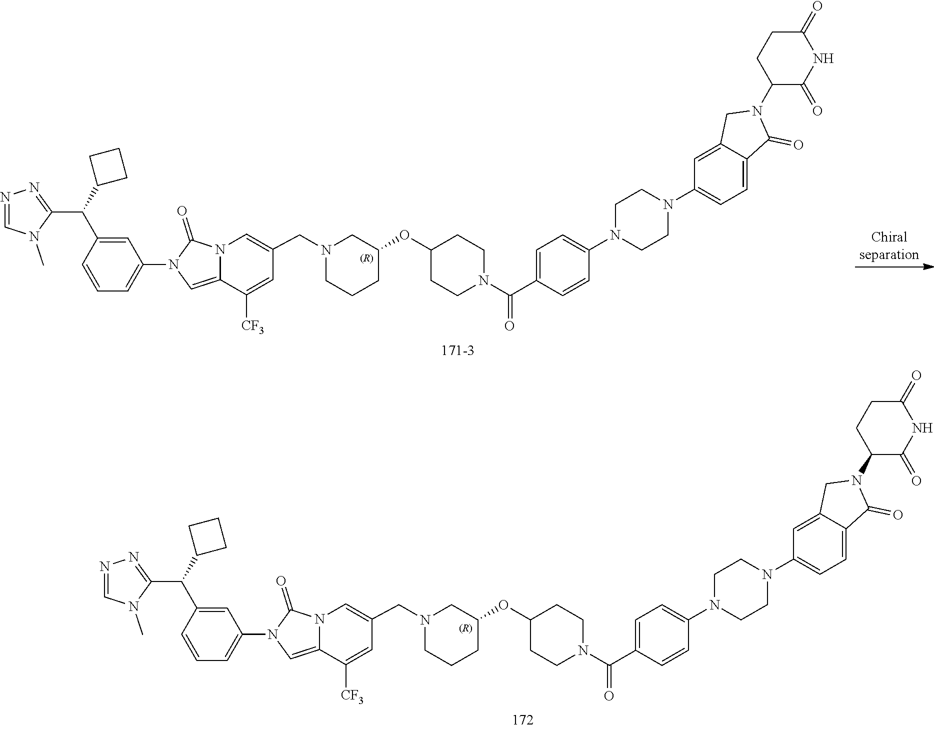

### Synthesis of Compound 172.

The Compound 171-3 (350 mg) was purified by Prep-CHIRAL-HPLC with the following conditions Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 90% B to 90% B in 20 min; Wave Length: 220/254 nm; RT1 (min): 9.82; RT2 (min): 17.01; Sample Solvent: DCM:ACN=1:1—HPLC; Injection Volume: 0.85 mL; Number of Runs: 5. The second peak was the product. This resulted in Compound 172 (100 mg, 27%) as a yellow solid.

LC-MS-Compound 172: (ES, m/z): [M+H]⁺ 1054

H-NMR-Compound 172: (400 MHz, DMSO-d6, δ ppm): 1.05-1.14 (m, 1H), 1.39-1.42 (m, 3H), 1.69-1.83 (m, 9H), 1.90-1.98 (m, 3H), 2.02-2.08 (m, 1H), 2.35-2.41 (m, 1H), 2.49-2.51 (m, 3H), 2.86-2.91 (m, 2H), 3.17-3.20 (m, 3H), 3.24-3.28 (m, 1H), 3.42-3.45 (m, 4H), 3.49-3.66 (m, 8H), 3.70-3.85 (m, 3H), 4.20-4.37 (m, 3H), 5.02-5.07 (m, 1H), 6.98-7.01 (m, 3H), 7.11-7.20 (m, 3H), 7.27-7.31 (m, 3H), 7.41-7.45 (m, 1H), 7.54-7.56 (m, 1H), 7.67-7.73 (m, 3H), 8.32 (s, 1H), 10.95 (s, 1H).

### Example 171. Compound 173

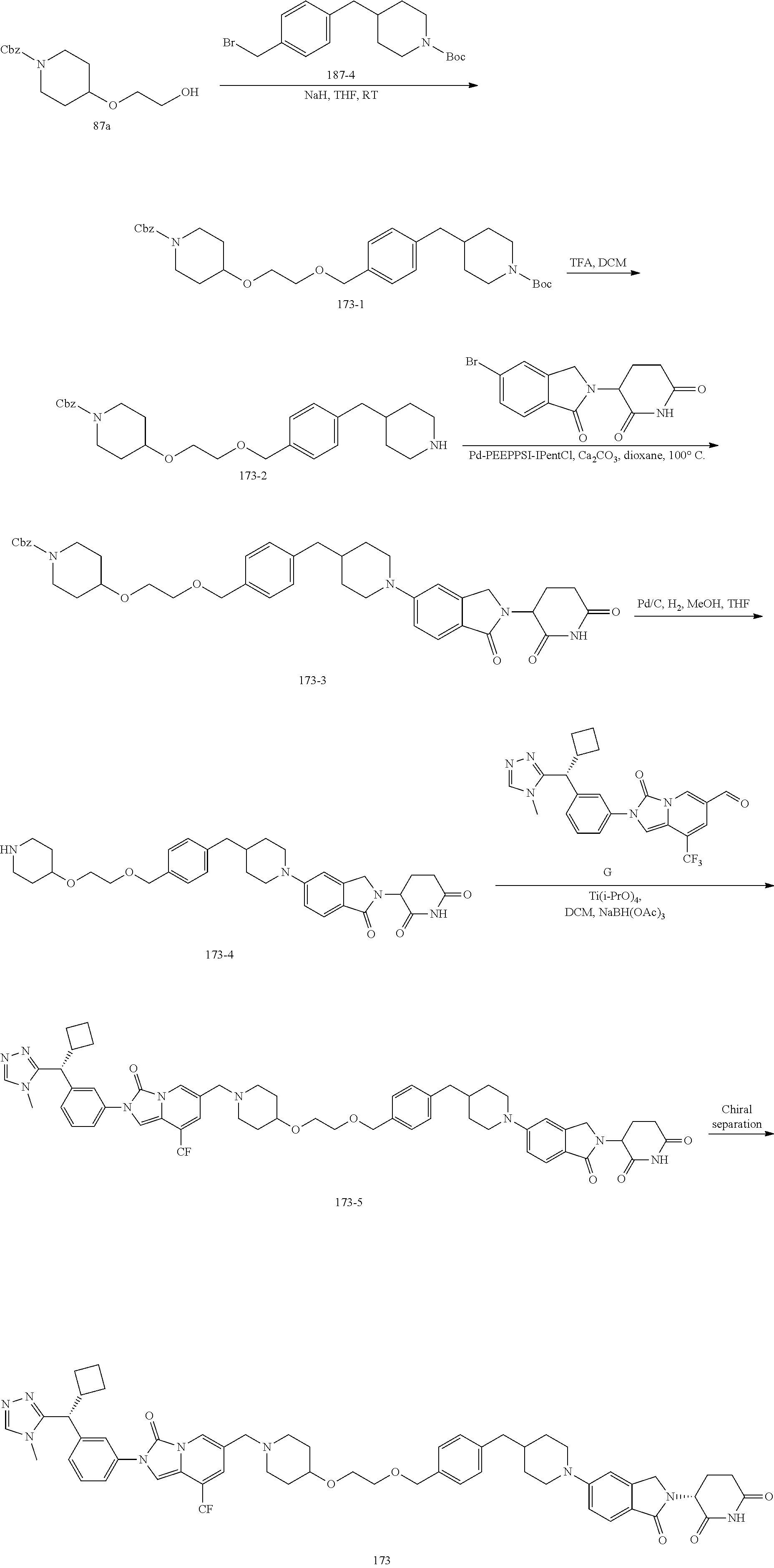

### Synthesis of Compound 173-1.

A solution of Compound 87a (3.4 g, 12.172 mmol, 1 equiv) in THF (34 mL) was treated with NaH (0.583 g, 14.606 mmol, 1.2 equiv, 60% in oil) for 1 h at 0° C. under nitrogen atmosphere followed by the addition of Compound 187-4 (6.72 g, 18.258 mmol, 1.5 equiv) in portions at room temperature. The resulting mixture was stirred for additional overnight at room temperature. The reaction was quenched by the addition of sat. NH₄Cl (aq.) (120 mL) at room temperature. The aqueous layer was extracted with EtOAc (6×250 mL). The resulting mixture was concentrated under reduced pressure. The residue was purified by silica gel column chromatography, eluted with PE/EA (1:1) to afford Compound 173-1 (4.5 g, 58.71%) as a light yellow oil.

### Synthesis of Compound 173-2.

To a stirred solution of Compound 173-1 (5.6 g, 9.881 mmol, 1 equiv) in DCM (46 mL) was added TFA (10 mL) in portions at room temperature under nitrogen atmosphere. The resulting mixture was stirred for additional 4 h at room temperature. The reaction was quenched by the addition of Na₂CO₃ (150 mL) at room temperature. The aqueous layer was extracted with DCM/MeOH=5/1) (3×250 mL). The resulting mixture was concentrated under reduced pressure, to afford Compound 173-2 (4.9 g, crude) as a light brown oil.

### Synthesis of Compound 173-3.

To a stirred mixture of Compound 173-2 (4.1 g, 8.787 mmol, 1 equiv) and 3-(5-bromo-1-oxo-3H-isoindol-2-yl)piperidine-2,6-dione (4.26 g, 13.181 mmol, 1.5 equiv) in dioxane (120 mL) were added Cs₂CO₃ (8.59 g, 26.361 mmol, 3 equiv) and Pd-PEPPSI-IPentCl 2-methylpyridine (o-picoline (0.74 g, 0.879 mmol, 0.1 equiv) under argon atmosphere. The resulting mixture was stirred for overnight at 100° C. under argon atmosphere. The reaction was quenched by the addition of Water/HOAc=10/1 (400 mL) at room temperature. The resulting mixture was extracted with CH₂Cl₂ (2×200 mL). The combined organic layers were concentrated under reduced pressure. The residue was purified by silica gel column chromatography, eluted with CH₂Cl₂/MeOH (50:1) to afford crude product (3.6 g). The crude product (3.6 g) was purified by reversed-phase flash chromatography with the following conditions: (column, C18 silica gel; mobile phase, MeCN in Water (0.1% FA), 5% to 65% gradient in 30 min; detector, UV 254 nm) to afford Compound 173-3 (1.6 g, 25.69%) as a white solid.

### Synthesis of Compound 173-4.

To a solution of Compound 173-3 (1.6 g, 2.257 mmol, 1 equiv) in MeOH (20 mL) and THF (60 mL) was added Pd/C (0.32 g, 10%) under nitrogen atmosphere in a 250 mL round-bottom flask. The mixture was hydrogenated at room temperature for 4 h under hydrogen atmosphere using a hydrogen balloon, filtered through a Celite pad and concentrated under reduced pressure. This resulted in Compound 173-4 (1.2 g, 92.50%) as a white solid.

### Synthesis of Compound 173-5.

To a stirred solution of Compound 173-4 (600 mg, 1.044 mmol, 1 equiv) and Intermediate G (475.47 mg, 1.044 mmol, 1 equiv) in DCM (12 mL) was added Ti(Oi-Pr)₄ (1186.88 mg, 4.176 mmol, 4 equiv) at room temperature under nitrogen atmosphere. The resulting mixture was stirred for 30 min at room temperature under nitrogen atmosphere. To the above mixture was added NaBH(OAc)₃ (663.79 mg, 3.132 mmol, 3 equiv). The resulting mixture was stirred for additional 5 h at room temperature. The reaction was quenched by the addition of Water/HOAc=10/1 (30 mL) at room temperature. The resulting mixture was extracted with CH₂Cl₂/MeOH=10/1 (3×40 mL). The combined organic layers were concentrated under reduced pressure. The residue was purified by reversed-phase flash chromatography with the following conditions: column, C18 silica gel; mobile phase, MeCN in Water (0.1% FA), 5% to 60% gradient in 10 min; detector, UV 254 nm. This resulted in Compound 173-5 (350 mg, 33.06%) as a yellow solid.

### Synthesis of Compound 173.

Compound 173-5 (350 mg, 0.345 mmol, 1 equiv) was purified by Prep-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 70% B to 70% B in 8 min; Wave Length: 220/254 nm; RT1 (min): 4.87; RT2 (min): 6.63; The first peak was product) to afford Compound 173 (89.9 mg, 25.69%) as a yellow solid.

LCMS-Compound 173: (ES, m/z): [M+H]⁺ 1014

NMR-Compound 173: (400 MHz, CD₃OD, δ ppm): δ1.26-1.40 (m, 2H), 1.70-1.76 (m, 6H), 1.88-1.90 (m, 6H), 2.04-2.12 (m, 1H), 2.16-2.24 (m, 1H), 2.25-2.32 (m, 2H), 2.34-2.49 (m, 1H), 2.56-2.58 (d, 2H), 2.77-2.84 (m, 6H), 3.38 (s, 2H), 3.46-3.48 (m, 2H), 3.52 (s, 3H), 3.62-3.63 (m, 4H), 3.85-3.88 (m, 2H), 4.24-4.35 (m, 3H), 4.53 (s, 2H), 5.05-5.09 (m, 1H), 7.00-7.02 (m, 2H), 7.09-7.10 (m, 2H), 7.15-7.17 (m, 2H), 7.23-7.28 (m, 3H), 7.44-7.48 (t, 1H), 7.56-7.66 (m, 4H), 8.31 (s, 1H).

### Example 172. Compound 174

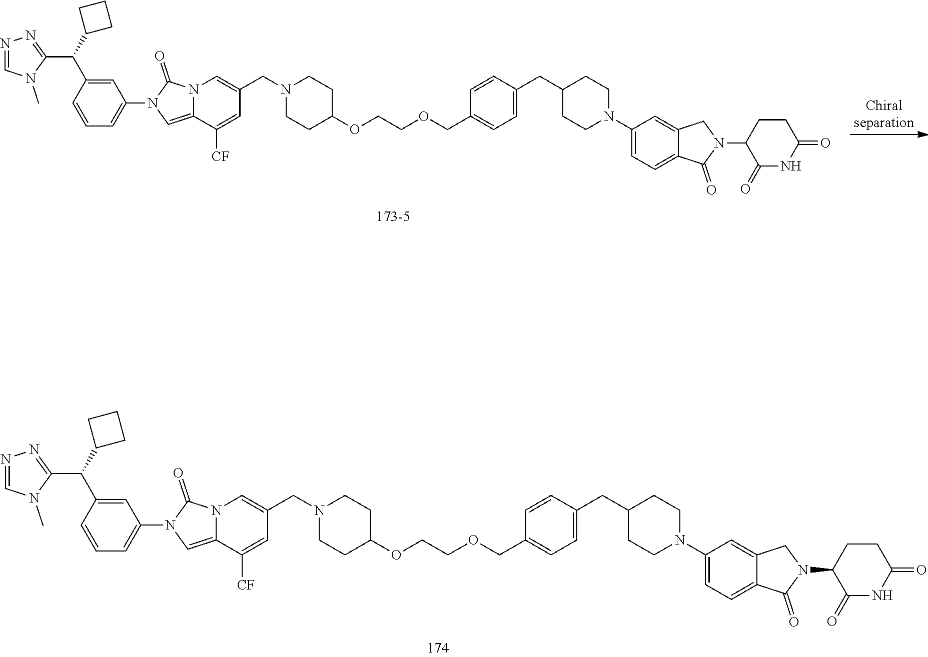

### Synthesis of Compound 174.

Compound 173-5 (350 mg) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 70% B to 70% B in 8 min; Wave Length: 220/254 nm; RT1 (min): 4.87; RT2 (min): 6.63; The second peak was product) to afford Compound 174 (96.7 mg, 27.63%) as a yellow solid.

LCMS-Compound 174: (ES, m/z): [M+H]⁺ 1014

NMR-Compound 174: (400 MHz, CD₃OD, δ ppm): δ1.29-1.35 (m, 2H), 1.70-1.74 (m, 6H), 1.87-1.90 (m, 6H), 2.04-2.12 (m, 1H), 2.16-2.24 (m, 1H), 2.25-2.49 (m, 3H), 2.56-2.58 (d, 2H), 2.77-2.89 (m, 6H), 3.19-3.21 (m, 1H), 3.48-3.52 (m, 7H), 3.62-3.65 (m, 4H), 3.85-3.88 (m, 2H), 4.25-4.28 (d, 1H), 4.34-4.36 (m, 2H), 4.53 (s, 2H), 5.05-5.09 (m, 1H), 7.00-7.03 (m, 2H), 7.09-7.12 (m, 2H), 7.15-7.17 (m, 2H), 7.24-7.29 (m, 3H), 7.44-7.48 (t, 1H), 7.56-7.64 (m, 3H), 7.71 (s, 1H), 8.31 (s, 1H).

### Example 173. Compound 175

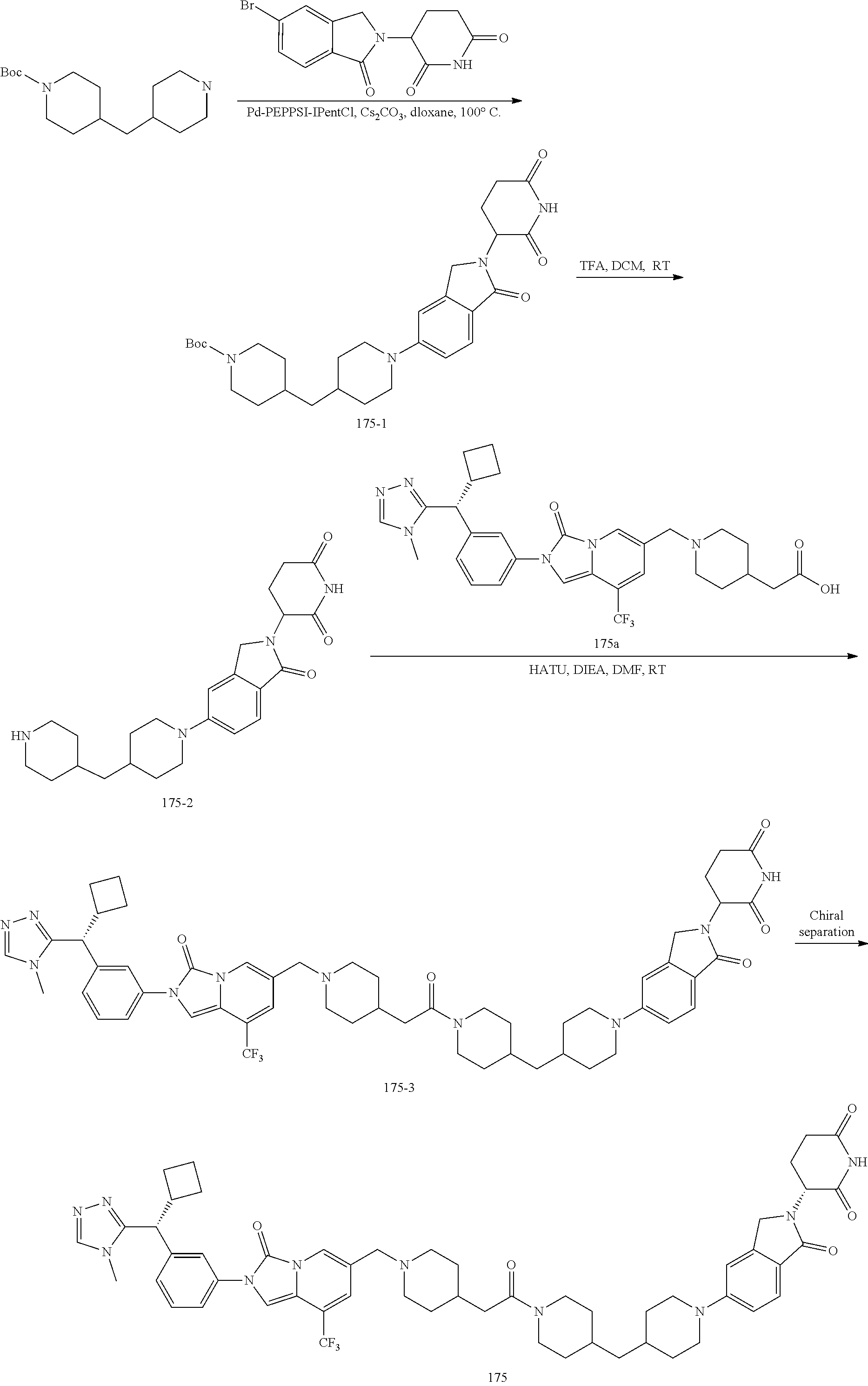

### Synthesis of Compound 175-1.

To a stirred solution of tert-butyl 4-(piperidin-4-ylmethyl)piperidine-1-carboxylate (2 g, 7.081 mmol, 1 equiv) and 3-(5-bromo-1-oxo-31H-isoindol-2-yl)piperidine-2,6-dione (3.43 g, 10.622 mmol, 1.5 equiv) in 1,4-dioxane (100 mL) was added Cs₂CO₃ (6.92 g, 21.243 mmol, 3 equiv) and Pd-PEPPSI-IPentCl 2-methylpyridine (o-picoline (0.60 g, 0.708 mmol, 0.1 equiv) at room temperature under nitrogen atmosphere. The resulting mixture was stirred for overnight at 100° C. under nitrogen atmosphere. The reaction was quenched with HOAc (20 mL) at room temperature. The resulting mixture was concentrated under vacuum. The residue was purified by silica gel column chromatography, eluted with DCM/MeOH (30:1) to afford Compound 175-1 (2.3 g, 55.71%) as a yellow solid.

### Synthesis of Compound 175-2.

A solution of Compound 175-1 (2.3 g, 4.384 mmol, 1 equiv) and TFA (5 mL) in DCM (30 mL) was stirred for 2 h at room temperature. The resulting mixture was concentrated under vacuum. The crude product was used in the next step directly without further purification. This resulted in Compound 175-2 (2.3 g, 98.87%) as a yellow solid.

### Synthesis of Compound 175-3.

To a stirred solution of Compound 175-2 (800 mg, 1.507 mmol, 1.76 equiv, 80%) and Compound 175a (500 mg, 0.858 mmol, 1.00 equiv) in DMF (15 mL) were added HATU (391.57 mg, 1.030 mmol, 1.20 equiv) and DIEA (332.75 mg, 2.574 mmol, 3 equiv) at room temperature. The resulting mixture was stirred for 3 h at room temperature. The residue was purified by reversed-phase flash chromatography with the following conditions (column, C18 silica gel; mobile phase, MeCN in Water (0.1% FA), 5% to 60% gradient in 40 min; detector, UV 254 nm) to afford the crude product. The crude product was purified by Prep-HPLC with the following conditions (Column: Sunfire prep C18 column, 30*150 mm, 5 μm; Mobile Phase A: Water (0.1% FA), Mobile Phase B: ACN; Flow rate: 60 mL/min; Gradient: 26% B to 36% B in 7 min, Wave Length: 254; 220 nm; RT1 (min): 5.3) to afford Compound 175-2 (140 mg, 16.00%) as a yellow solid.

### Synthesis of Compound 175.

The Compound 175-3 (140 mg) was purified by Prep-Chiral-HPLC with the following conditions (Column: CHIRALPAK IH, 2*25 cm, 5 μm; Mobile Phase A: Hex (0.2% TEA), Mobile Phase B: DCM:ACN=1:1; Flow rate: 20 mL/min; Gradient: 90% B to 90% B in 13 min; Wave Length: 220/254 nm; RT1 (min): 7.34; RT2 (min): 11.71; the first peak is product) to afford the crude product. The crude product was purified by reverse flash chromatography with the following conditions (column, C18 silica gel; mobile phase, MeCN in water (0.1% FA), 0% to 40% gradient in 10 min; detector, UV 254 nm) to afford Compound 175 (55.1 mg, 38.53%) as a yellow solid.

LC-MS-Compound 175: (ES, m/z): [M+H]⁺ 989

H-NMR-Compound 175: (400 MHz, DMSO-d₆, δ ppm): 0.85.95 (m, 1H), 0.95-1.05 (m, 1H), 1.13-1.29 (m, 6H), 1.55-1.88 (m, 15H), 1.91-2.29 (m, 6H), 2.41-2.50 (m, 1H), 2.50-2.67 (m, 1H), 2.77-3.02 (m, 6H), 3.21-3.25 (m, 3H), 3.43 (s, 3H), 3.85-3.93 (m, 3H), 4.17-4.45 (m, 4H), 5.02-5.07 (d, 1H), 6.95-7.04 (m, 3H), 7.19-7.21 (d, 1H), 7.32 (s, 1H), 7.42-7.50 (m, 2H), 7.68-7.73 (m, 3H), 8.33 (s, 1H), 10.95 (s, 1H).

### Example 174. Compound 176

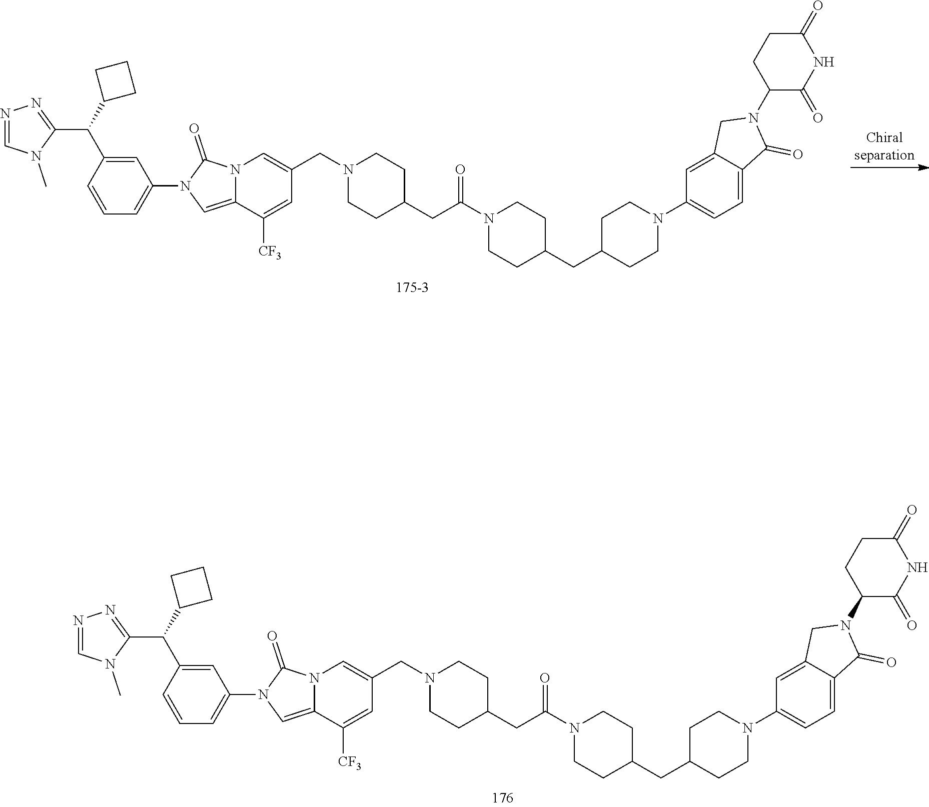 [SMILES: Cn1cnnc1[C@@H](c1cccc(-n2cc3c(C(F)(F)F)cc(CN4CCC(CC(=O)N5CCC(CC6CCN(c7ccc8c(c7)CN(C7CCC(=O)NC7=O)C8=O)CC6)CC5)CC4)cn3c2=O)c1)C1CCC1 ; Cn1cnnc1[C@@H](c1cccc(-n2cc3c(C(F)(F)F)cc(CN4CCC(CC(=O)N5CCC(CC6CCN(c7ccc8c(c7)CN([C@H]7CCC(=O)NC7=O)C8=O)CC6)CC5)CC4)cn3c2=O)c1)C1CCC1]

### Synthesis of Compound 176.

The Compound 175-3 (140 mg, 0.142 mmol, 1 equiv) was purified by Prep-Chiral-HPLC with the following conditions (Column: CHIRALPAK IH, 2*25 cm, 5 μm; Mobile Phase A: Hex (0.2% TEA), Mobile Phase B: DCM:ACN=1:1; Flow rate: 20 mL/min; Gradient: 90% B to 90% B in 13 min; Wave Length: 220/254 nm; RT1 (min): 7.34; RT2 (min): 11.71; the second peak is product) to afford the crude product. The crude product was purified by reverse flash chromatography with the following conditions (column, C18 silica gel; mobile phase, MeCN in water (0.1% FA), 0% to 40% gradient in 10 min; detector, UV 254 nm) to afford Compound 176 (52.5 mg, 36.86%) as a yellow solid.

LC-MS-Compound 176: (ES, m/z): [M+H]⁺ 989

H-NMR-Compound 176: (400 MHz, DMSO-d₆, δ ppm): 0.85-1.05 (m, 2H), 1.13-1.29 (m, 6H), 1.55-1.88 (m, 15H), 1.91-2.13 (m, 4H), 2.21-2.29 (m, 2H), 2.41-2.50 (m, 1H), 2.50-2.67 (m, 1H), 2.77-3.02 (m, 6H), 3.21-3.25 (m, 3H), 3.43 (s, 3H), 3.85-3.93 (m, 3H), 4.17-4.45 (m, 4H), 5.02-5.07 (d, 1H), 6.98-7.04 (m, 3H), 7.19-7.21 (d, 1H), 7.32 (s, 1H), 7.42-7.50 (m, 2H), 7.68-7.73 (m, 3H), 8.33 (s, 1H), 10.95 (s, 1H).

### Example 175. Compound 177

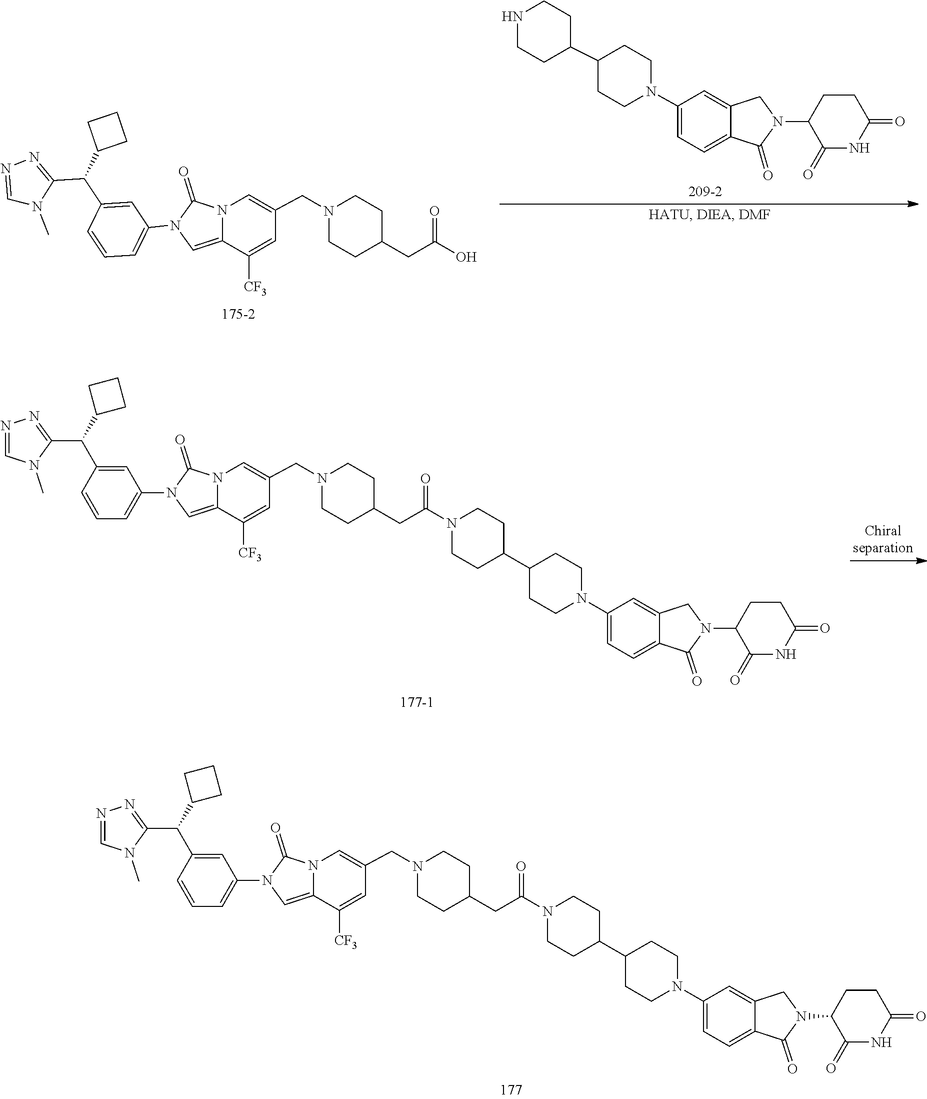 [SMILES: Cn1cnnc1[C@@H](c1cccc(-n2cc3c(C(F)(F)F)cc(CN4CCC(CC(=O)O)CC4)cn3c2=O)c1)C1CCC1 ; O=C1CCC(N2Cc3cc(N4CCC(C5CCNCC5)CC4)ccc3C2=O)C(=O)N1 ; Cn1cnnc1[C@@H](c1cccc(-n2cc3c(C(F)(F)F)cc(CN4CCC(CC(=O)N5CCC(C6CCN(c7ccc8c(c7)CN(C7CCC(=O)NC7=O)C8=O)CC6)CC5)CC4)cn3c2=O)c1)C1CCC1 ; Cn1cnnc1[C@@H](c1cccc(-n2cc3c(C(F)(F)F)cc(CN4CCC(CC(=O)N5CCC(C6CCN(c7ccc8c(c7)CN([C@@H]7CCC(=O)NC7=O)C8=O)CC6)CC5)CC4)cn3c2=O)c1)C1CCC1]

### Synthesis of Compound 177-1.

To a stirred solution of Compound 175-2 (500 mg, 0.85 mmol, 1.0 equiv) and DIEA (221 mg, 1.71 mmol, 2.0 equiv) in DMF (5 mL) was added HATU (489 mg, 1.28 mmol, 1.5 equiv) at room temperature. The resulting mixture was stirred for 30 min at room temperature. To the above mixture was added Compound 209-2 (422 mg, 1.03 mmol, 1.2 equiv) at room temperature. The resulting mixture was stirred for additional overnight at room temperature. The reaction was quenched by the addition of sat. NH₄Cl (aq.) (20 mL) at room temperature. The aqueous layer was extracted with DCM:MeOH (5:1) (3×30 ml). The residue was purified by reversed-phase flash chromatography with the following conditions: column, C18 silica gel; mobile phase, MeCN in water (0.1% FA), 10% to 50% gradient in 30 min; detector, UV 254 nm. This resulted in Compound 177-1 (450 mg, 54%) as a yellow solid.

### Synthesis of Compound 177.

The Compound 177-1 (460 mg) was purified by Prep-CHIRAL-HPLC with the following conditions Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 90% B to 90% B in 12 min; Wave Length: 220/254 nm; RT1 (min): 6.78; RT2 (min): 9.22; Sample Solvent: DCM:ACN=1:1—HPLC; Injection Volume: 1.2 mL; Number Of Runs: 10. The first peak was the product. This resulted in Compound 177 (141 mg, 31%) as a yellow solid.

LC-MS-Compound 177: (ES, m/z): [M+H]⁺ 975

H-NMR-Compound 177: (400 MHz, DMSO-d6, δ ppm): 0.95-1.06 (m, 2H), 1.21-1.27 (m, 7H), 1.67-1.81 (m, 12H), 1.95-1.97 (m, 2H), 2.00-2.05 (m, 1H), 2.22-2.23 (m, 2H), 2.49-2.50 (m, 2H), 2.51-2.55 (m, 2H), 2.56-2.58 (m, 1H), 2.70-2.74 (m, 2H), 2.85-2.91 (m, 4H), 3.32-3.35 (m, 2H), 3.36-3.37 (m, 1H), 3.42 (s, 3H), 3.88-3.91 (m, 3H), 4.19-4.27 (m, 3H), 4.29-4.33 (m, 1H), 4.98-5.02 (m, 1H), 7.01-7.04 (m, 3H), 7.18-7.20 (d, 1H), 7.31 (s, 1H), 7.41-7.50 (m, 2H), 7.67-7.72 (m, 3H), 8.32 (s, 1H), 10.95 (s, 1H).

### Example 176. Compound 178

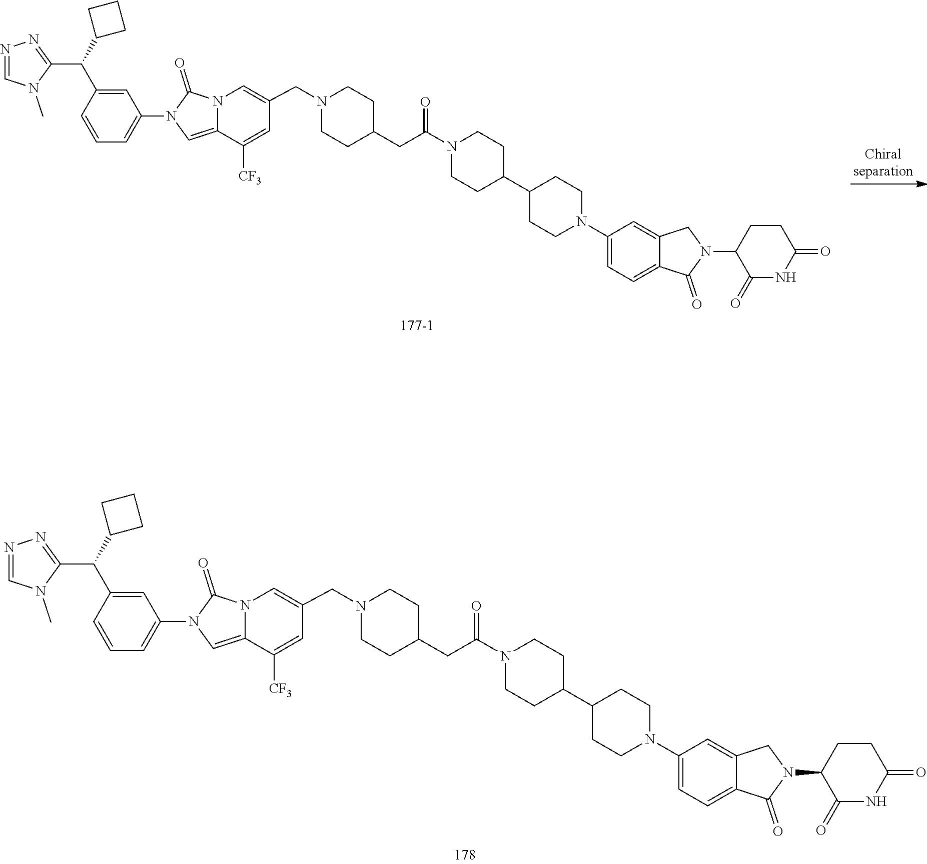 [SMILES: Cn1cnnc1[C@@H](c1cccc(-n2cc3c(C(F)(F)F)cc(CN4CCC(CC(=O)N5CCC(C6CCN(c7ccc8c(c7)CN(C7CCC(=O)NC7=O)C8=O)CC6)CC5)CC4)cn3c2=O)c1)C1CCC1 ; Cn1cnnc1[C@@H](c1cccc(-n2cc3c(C(F)(F)F)cc(CN4CCC(CC(=O)N5CCC(C6CCN(c7ccc8c(c7)CN([C@H]7CCC(=O)NC7=O)C8=O)CC6)CC5)CC4)cn3c2=O)c1)C1CCC1]

### Synthesis of Compound 178.

The Compound 177-1 (460 mg) was purified by Prep-CHIRAL-HPLC with the following conditions Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 90% B to 90% B in 12 min; Wave Length: 220/254 nm; RT1 (min): 6.78; RT2 (min): 9.22; Sample Solvent: DCM:ACN=1:1—HPLC; Injection Volume: 1.2 mL; Number Of Runs: 10. The second peak was the product. This resulted in Compound 178 (164 mg, 36%) as a yellow solid.

LC-MS-Compound 178: (ES, m/z): [M+H]⁺ 975

H-NMR-Compound 178: (400 MHz, DMSO-d6, δ ppm): 0.95-1.06 (m, 2H), 1.21-1.27 (m, 7H), 1.67-1.81 (m, 12H), 1.95-1.97 (m, 2H), 2.00-2.05 (m, 1H), 2.22-2.23 (m, 2H), 2.49-2.50 (m, 2H), 2.51-2.55 (m, 2H), 2.56-2.58 (m, 1H), 2.70-2.74 (m, 2H), 2.85-2.91 (m, 4H), 3.32-3.35 (m, 2H), 3.36-3.37 (m, 1H), 3.42 (s, 3H), 3.88-3.91 (m, 3H), 4.19-4.27 (m, 3H), 4.29-4.33 (m, 1H), 4.98-5.02 (m, 1H), 7.01-7.04 (m, 3H), 7.18-7.20 (d, 1H), 7.31 (s, 1H), 7.41-7.50 (m, 2H), 7.67-7.72 (m, 3H), 8.32 (s, 1H), 10.95 (s, 1H).

### Example 177. Compound 179

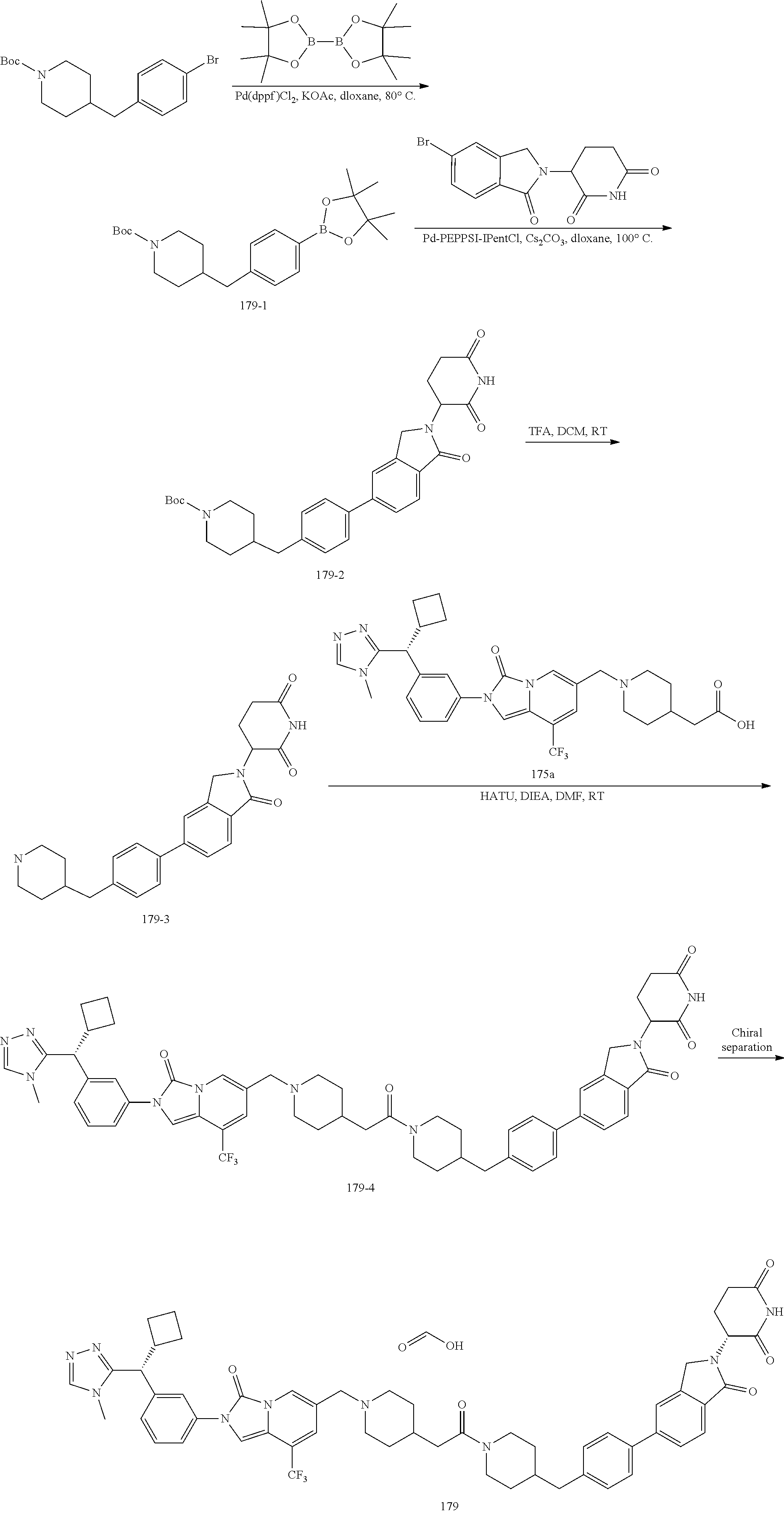

### Synthesis of Compound 179-1.

To a stirred solution of tert-butyl 4-[(4-bromophenyl)methyl]piperidine-1-carboxylate (5.5 g, 15.524 mmol, 1 equiv) and bis(pinacolato)diboron (5.91 g, 23.286 mmol, 1.5 equiv) in dioxane (100 mL) was added KOAc (4.57 g, 46.572 mmol, 3 equiv) and Pd(dppf)Cl₂ (1.14 g, 1.552 mmol, 0.1 equiv) at room temperature under nitrogen atmosphere. The resulting mixture was stirred overnight at 80° C. under nitrogen atmosphere. The resulting mixture was diluted with water (500 mL). The aqueous layer was extracted with EtOAc (2×200 mL). The resulting mixture was concentrated under vacuum. The residue was purified by silica gel column chromatography, eluted with PE/EA (10:1) to afford Compound 179-1 (5.5 g, 79.45%) as an off-white solid.

### Synthesis of Compound 179-2.

To a stirred solution of Compound 179-1 (5.5 g, 13.704 mmol, 1 equiv) and 3-(5-bromo-1-oxo-3H-isoindol-2-yl)piperidine-2,6-dione (6.64 g, 20.556 mmol, 1.5 equiv) in dioxane (100 mL) was added Cs₂CO₃ (13.39 g, 41.112 mmol, 3 equiv) and Pd-PEPPSI-IPentCl 2-methylpyridine (o-picoline (1.15 g, 1.370 mmol, 0.1 equiv) at room temperature under nitrogen atmosphere. The resulting mixture was stirred overnight at 100° C. under nitrogen atmosphere. The reaction was quenched with HOAc (40 mL) at room temperature. The resulting mixture was concentrated under vacuum. The residue was purified by silica gel column chromatography, eluted with DCM (0.1% HOAc)/MeOH (30:1) to afford Compound 179-2 (5 g, 42.29%) as a yellow solid.

### Synthesis of Compound 179-3.

A solution of Compound 179-2 (5 g, 5.796 mmol, 1 equiv, 60%) and TFA (10 mL) in DCM (50 mL) was stirred for 3 h at room temperature. The resulting mixture was concentrated under vacuum. The residue was purified by silica gel column chromatography, eluted with DCM/MeOH (15:1) to afford Compound 179-3 (1 g, 37.19%) as a light yellow solid.

### Synthesis of Compound 179-4.

To a stirred solution of Compound 179-3 (500 mg, 1.198 mmol, 1 equiv) and Compound 175a (697.74 mg, 1.198 mmol, 1 equiv) in DMF (15 mL) were added HATU (546.43 mg, 1.438 mmol, 1.2 equiv) and DIEA (464.35 mg, 3.594 mmol, 3 equiv) at room temperature. The resulting mixture was stirred for 3 h at room temperature. The residue was purified by reversed-phase flash chromatography with the following conditions (column, C18 silica gel; mobile phase, MeCN in Water (0.1% FA), 10% to 70% gradient in 40 min; detector, UV 254 nm) to afford the crude product. The crude product was purified by Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 25% B to 39% B in 8 min, Wave Length: 254; 220 nm; RT1 (min): 7.2) to afford Compound 179-4 (280 mg, 23.09%) as a yellow solid.

### Synthesis of Compound 179.

The Compound 179-4 (280 mg) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 90% B to 90% B in 10.5 min; Wave Length: 220/254 nm; RT1 (min): 6.55; RT2 (min): 8.77; the first peak is product) to afford the crude product. The crude product was purified by reverse flash chromatography with the following conditions (column, C18 silica gel; mobile phase, MeCN in water (0.1% FA), 0% to 40% gradient in 10 min; detector, UV 254 nm) to afford Compound 179 (92.4 mg, 29.98%) as a yellow solid.

LC-MS-Compound 179: (ES, m/z): [M−HCOOH+H]⁺ 982

H-NMR-Compound 179: (400 MHz, DMSO-d₆, δ ppm): 0.95-1.29 (m, 4H), 1.55-1.79 (m, 6H), 1.79-1.85 (m, 5H), 1.91-2.13 (m, 4H), 2.21-2.25 (m, 2H), 2.41-2.50 (m, 2H), 2.50-2.67 (m, 3H), 2.78-2.85 (m, 2H), 2.91-2.95 (m, 2H), 3.21-3.25 (m, 3H), 3.43 (s, 3H), 3.85-3.93 (m, 1H), 4.36-4.41 (m, 2H), 4.49-4.53 (m, 1H), 5.12-5.17 (d, 1H), 7.00 (s, 1H), 7.18-7.20 (d, 1H), 7.30-7.32 (m, 3H), 7.42-7.46 (m, 1H), 7.65-7.73 (m, 4H), 7.73-7.79 (m, 3H), 7.88 (s, 1H), 8.15 (s, 1H), 8.33 (s, 1H), 11.05 (s, 1H).

### Example 178. Compound 180

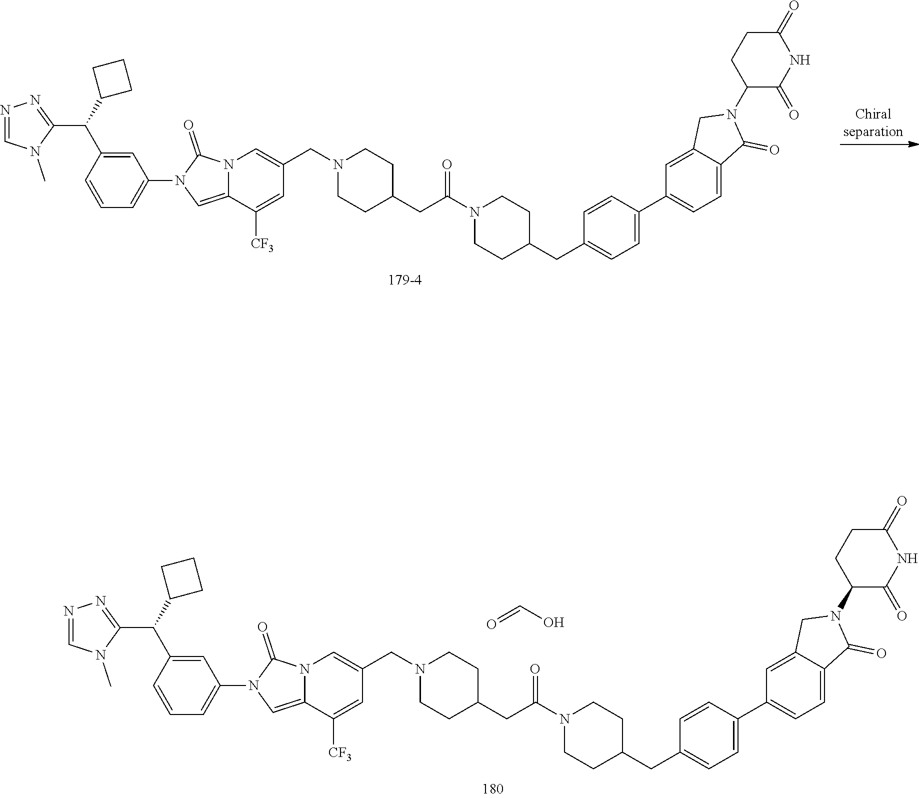 [SMILES: Cn1cnnc1[C@@H](c1cccc(-n2cc3c(C(F)(F)F)cc(CN4CCC(CC(=O)N5CCC(Cc6ccc(-c7ccc8c(c7)CN(C7CCC(=O)NC7=O)C8=O)cc6)CC5)CC4)cn3c2=O)c1)C1CCC1 ; Cn1cnnc1[C@@H](c1cccc(-n2cc3c(C(F)(F)F)cc(CN4CCC(CC(=O)N5CCC(Cc6ccc(-c7ccc8c(c7)CN([C@H]7CCC(=O)NC7=O)C8=O)cc6)CC5)CC4)cn3c2=O)c1)C1CCC1 ; O=CO]

### Synthesis of Compound 180.

The Compound 179-4 (280 mg) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 90% B to 90% B in 10.5 min; Wave Length: 220/254 nm; RT1 (min): 6.55; RT2 (min): 8.77; the second peak is product) to afford the crude product. The crude product was purified by reverse flash chromatography with the following conditions (column, C18 silica gel; mobile phase, MeCN in water (0.1% FA), 0% to 40% gradient in 10 min; detector, UV 254 nm) to afford Compound 180 (86.0 mg, 28.84%) as a yellow solid.

LC-MS-Compound 180: (ES, m/z): [M−HCOOH+H]⁺ 982

H-NMR-Compound 180: (400 MHz, DMSO-d₆, δ ppm): 0.95-1.29 (m, 4H), 1.55-1.79 (m, 6H), 1.79-1.85 (m, 5H), 1.91-2.13 (m, 4H), 2.21-2.25 (m, 2H), 2.41-2.50 (m, 2H), 2.50-2.67 (m, 3H), 2.78-2.85 (m, 2H), 2.91-2.95 (m, 2H), 3.21-3.25 (m, 3H), 3.43 (s, 3H), 3.85-3.93 (m, 1H), 4.36-4.41 (m, 2H), 4.49-4.53 (m, 1H), 5.12-5.17 (d, 1H), 7.00 (s, 1H), 7.18-7.20 (d, 1H), 7.30-7.32 (m, 3H), 7.42-7.46 (m, 1H), 7.65-7.79 (m, 7H), 7.88 (s, 1H), 8.15 (s, 1H), 8.33 (s, 1H), 11.05 (s, 1H).

### Example 179. Compound 181

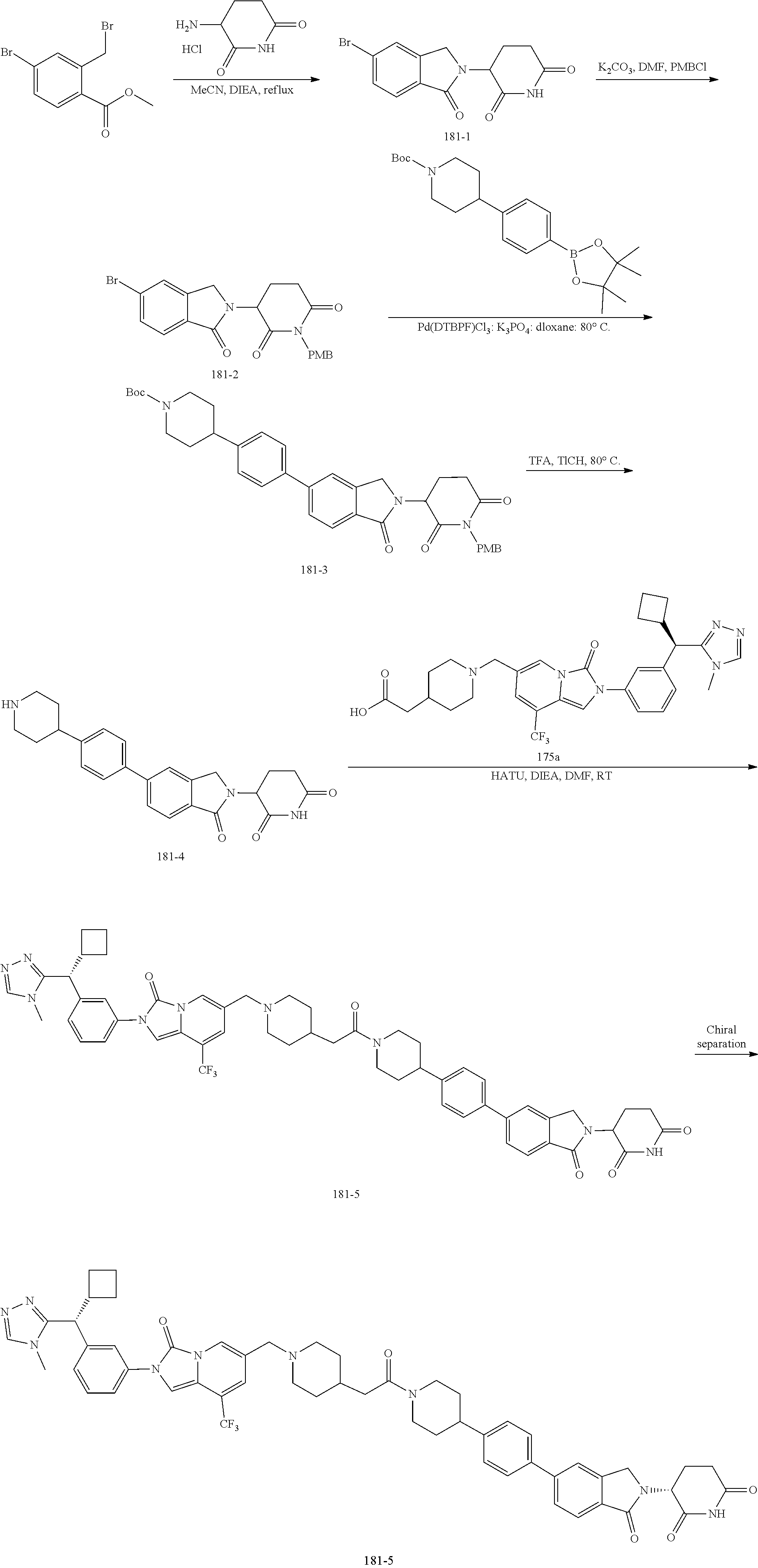

### Synthesis of Compound 181-1.

To a stirred solution of methyl 4-bromo-2-(bromomethyl)benzoate (187.1 g, 607.57 mmol, 1.0 equiv) and DIEA (235.6 g, 1822.71 mmol, 3.0 equiv) in MeCN (1000 mL) was added 3-aminopiperidine-2,6-dione hydrochloride (100 g, 607.57 mmol, 1.0 equiv) at room temperature. The resulting mixture was stirred for overnight at 80° C. under nitrogen atmosphere. The mixture was allowed to cool down to 0° C. The precipitated solids were collected by filtration and washed with MeCN (3×100 mL). This resulted in Compound 181-1 (162 g, 80%) as a purple solid.

### Synthesis of Compound 181-2.

To a stirred solution of Compound 181-1 (20.5 g, 63.44 mmol, 1.0 equiv) and K₂CO₃ (17.5 g, 126.88 mmol, 2.0 equiv) in DMF (200 mL) was added PMBCl (14.9 g, 95.16 mmol, 1.5 equiv) at room temperature under nitrogen atmosphere. The resulting mixture was stirred for overnight at 40° C. under nitrogen atmosphere. The resulting mixture was diluted with water (600 mL). The precipitated solids were collected by filtration and washed with water (2×100 mL). This resulted in Compound 181-2 (28.0 g, 96%) as a purple solid.

### Synthesis of Compound 181-3.

To a stirred solution of Compound 181-2 (2.0 g, 4.51 mmol, 1.0 equiv) and tert-butyl 4-[4-(4,4,5,5-tetramethyl-1,3,2-dioxaborolan-2-yl)phenyl]piperidine-1-carboxylate (1.7 g, 4.51 mmol, 1.0 equiv) in 1,4-dioxane (16 mL) and water (4 mL) were added Pd(DTBPF)Cl₂ (290 mg, 0.45 mmol, 0.1 equiv) and K₃PO₄ (1.9 g, 9.02 mmol, 2.0 equiv) at room temperature under nitrogen atmosphere. The resulting mixture was stirred for 2 h at 80° C. under nitrogen atmosphere. The reaction was quenched by the addition of water (60 mL) at room temperature. The aqueous layer was extracted with CH₂Cl₂ (3×30 mL). The resulting mixture was concentrated under reduced pressure. The residue was purified by silica gel column chromatography, eluted with CH₂Cl₂/MeOH (80:1) to afford Compound 181-3 (2.5 g, 84%) as a brown oil.

### Synthesis of Compound 181-4.

To a stirred solution of Compound 181-3 (2.4 g, 3.91 mmol, 1.0 equiv) in TFA (20 mL) were added CF₃SO₃H (5.9 g, 39.12 mmol, 10.0 equiv) at room temperature. The resulting mixture was stirred for overnight at 60° C. under nitrogen atmosphere. The resulting mixture was diluted with water (20 mL). The resulting mixture was concentrated under reduced pressure. The mixture was acidified to pH 5 with saturated Na₂CO₃ (aq.). The resulting mixture was concentrated under reduced pressure. The residue was purified by reversed-phase flash chromatography with the following conditions: column, C18 silica gel; mobile phase, MeCN in water (0.1% FA), 10% to 50% gradient in 30 min; detector, UV 254 nm. This resulted in Compound 181-4 (1.1 g, 69%) as a white solid.

### Synthesis of Compound 181-5.

To a stirred solution of Compound 175a (500 mg, 0.86 mmol, 1.0 equiv) in DMF (5 mL) were added DIEA (332.8 mg, 2.574 mmol, 3.0 equiv), HATU (652.6 mg, 1.72 mmol, 2.0 equiv) and Compound 181-4 (346.3 mg, 0.86 mmol, 1.0 equiv) at room temperature under nitrogen atmosphere. The resulting mixture was stirred for overnight at room temperature under nitrogen atmosphere. The reaction was quenched by the addition of water/AcOH=10:1 (60 mL) at room temperature. The aqueous layer was extracted with CH₂Cl₂/MeOH=5:1 (3×50 mL). The resulting mixture was concentrated under reduced pressure. The residue was purified by reversed-phase flash chromatography with the following conditions: column, C18 silica gel; mobile phase, MeCN in water (0.1% FA), 10% to 50% gradient in 30 min; detector, UV 254 nim. This resulted in Compound 181-5 (350 mg, 41%) as a yellow solid.

### Synthesis of Compound 181.

The Compound 181-5 (350 mg, 0.36 mmol, 1.0 equiv) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 90% B to 90% B in 15 min; Wave Length: 220/254 nm; RT1 (min): 7.23; RT2 (min): 11.24; Sample Solvent: DCM:ACN=1:1; the first peak was product) to afford crude product (170 mg). The residue was purified by reversed-phase flash chromatography with the following conditions: column, C18 silica gel; mobile phase, MeCN in water (0.1% FA), 10% to 50% gradient in 25 min; detector, UV 254 nm. This resulted in Compound 181 (105 mg, 28%) as a yellow solid.

LCMS-Compound 181: (ES, m/z): [M−HCOOH+H]⁺ 968

NMR-Compound 181: (400 MHz, DMSO-d6, δ ppm): 1.11-1.22 (m, 2H), 1.42-1.67 (m, 2H), 1.69-1.74 (m, 3H), 1.79-1.98 (m, 6H), 2.00-2.28 (m, 4H), 2.29-2.30 (m, 2H), 2.49-2.50 (m, 1H), 2.51-2.63 (m, 2H), 2.83-2.85 (m, 4H), 2.86-29.3 (m, 1H), 3.21-3.23 (m, 1H), 3.27-3.32 (m, 1H), 3.33-3.42 (m, 2H), 3.43 (s, 3H), 4.24-4.27 (d, 1H), 4.36-4.40 (d, 1H), 4.48 (s, 1H), 4.49-4.53 (m, 2H), 5.13-5.15 (m, 1H), 7.01 (s, 1H), 7.18-7.20 (d, 1H), 7.31 (s, 1H), 7.37-7.39 (d, 2H), 7.42-7.46 (t, 1H), 7.65-7.66 (m, 4H), 7.67 (s, 1H), 7.70-7.73 (d, 2H), 7.87 (s, 1H), 8.16 (s, 1H), 8.32 (s, 1H), 11.00 (s, 1H).

### Example 180. Compound 182

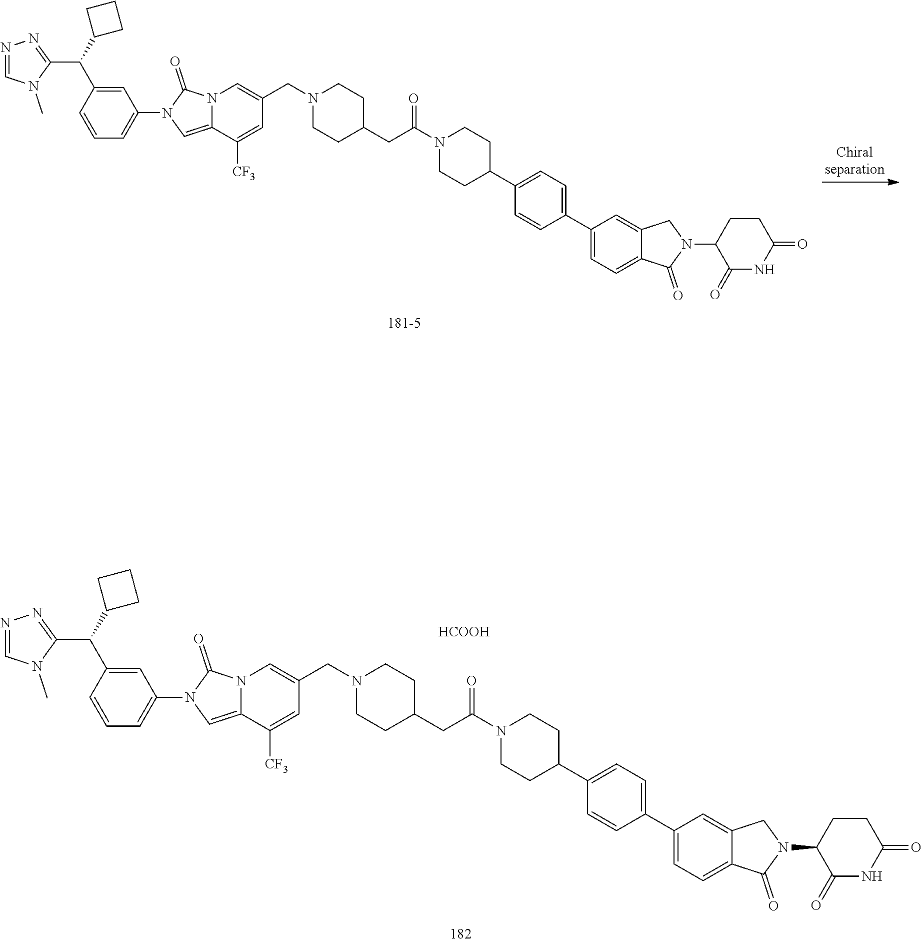 [SMILES: Cn1cnnc1[C@@H](c1cccc(-n2cc3c(C(F)(F)F)cc(CN4CCC(CC(=O)N5CCC(c6ccc(-c7ccc8c(c7)CN(C7CCC(=O)NC7=O)C8=O)cc6)CC5)CC4)cn3c2=O)c1)C1CCC1 ; Cn1cnnc1[C@@H](c1cccc(-n2cc3c(C(F)(F)F)cc(CN4CCC(CC(=O)N5CCC(c6ccc(-c7ccc8c(c7)CN([C@H]7CCC(=O)NC7=O)C8=O)cc6)CC5)CC4)cn3c2=O)c1)C1CCC1 ; "HCOOH"]

### Synthesis of Compound 182.

The Compound 181-5 (350 mg, 0.36 mmol, 1.0 equiv) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 90% B to 90% B in 15 min; Wave Length: 220/254 nm; RT1 (min): 7.23; RT2 (min): 11.24; the second peak was product) to afford crude product. The residue was purified by reversed-phase flash chromatography with the following conditions: column, C18 silica gel; mobile phase, MeCN in water (0.1% FA), 10% to 50% gradient in 25 min; detector, UV 254 nm. This resulted in Compound 182 (102 mg, 27%) as a yellow solid.

LCMS-Compound 182: (ES, m/z): [M−HCOOH+H]⁺ 968

NMR-Compound 182: (400 MHz, DMSO-d6, δ ppm): 1.22-1.23 (m, 2H), 1.42-1.49 (m, 1H), 1.51-1.59 (m, 1H), 1.67-1.70, (m, 3H), 1.74-1.83 (m, 4H), 1.96-2.01 (m, 2H), 2.29-2.30 (m, 4H), 2.49-2.50 (d, 2H), 2.50-2.51 (m, 1H), 2.60-2.63 (m, 2H), 2.84-2.89 (m, 4H), 2.91-3.01 (m, 1H), 3.11-3.21 (m, 1H), 3.23-3.25 (m, 1H), 3.28-3.32 (m, 2H), 3.43 (s, 3H), 4.02-4.08 (m, 1H), 4.24-4.27 (d, 1H), 4.36-4.40 (m, 1H), 4.49-4.53 (m, 2H), 5.13-5.15 (m, 1H), 7.01 (s, 1H), 7.18-7.20 (m, 1H), 7.31 (s, 1H), 7.37-7.39 (d, 2H), 7.42-7.44 (t, 1H), 7.66-7.67 (m, 4H), 7.68-7.69 (d, 1H), 7.70-7.73 (d, 2H), 7.88 (s, 1H), 8.15 (s, 1H), 8.32 (s, 1H), 11.00 (s, 1H).

### Example 181. Compound 183

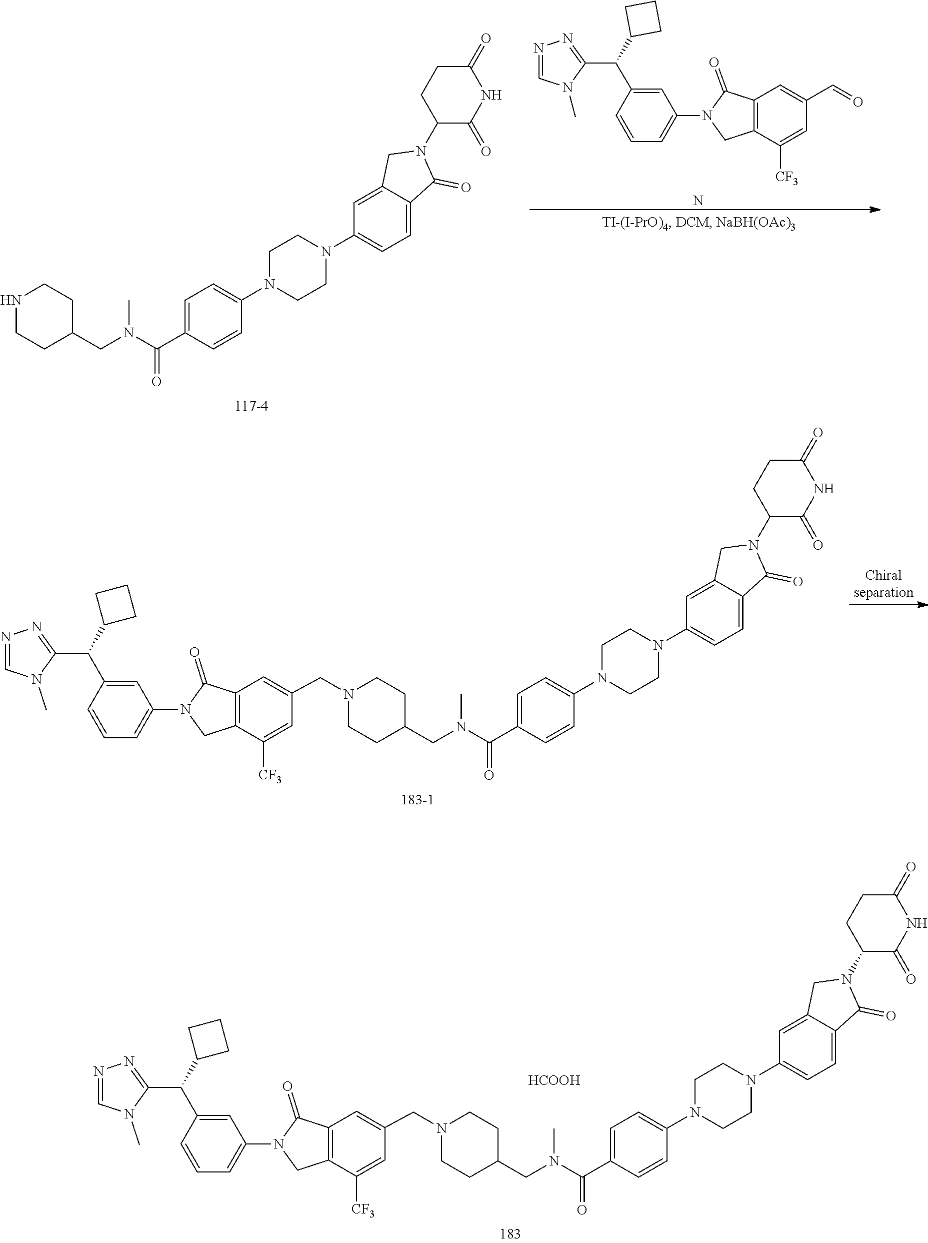

### Synthesis of Compound 183-1.

To a stirred solution of Compound 117-4 (1.2 g, 2.148 mmol, 1 equiv) and Intermediate N (1.07 g, 2.363 mmol, 1.1 equiv) in DCM (20 mL) were added Ti(Oi-Pr)₄ (1.83 g, 6.444 mmol, 3 equiv) at room temperature. The resulting mixture was stirred for overnight at room temperature. To the above mixture was added STAB (0.91 g, 4.296 mmol, 2 equiv) at room temperature. The resulting mixture was stirred for 2 h at room temperature. The mixture was acidified to pH 5 with HOAc (2 ml). The resulting mixture was concentrated under reduced pressure. The crude product (700 mg) was purified by Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 20% B to 33% B in 8 min, 33% B; Wave Length: 220; 254 nm; RT1 (min): 7.85) to afford Compound 183-1 (300 mg, 13.45%) as a white solid.

### Synthesis of Compound 183.

The Compound 183-1 (300 mg) was purified by Prep-Chiral-HPLC with the following conditions (Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 90% B to 90% B in 25 min; Wave Length: 220/254 nm; RT1 (min): 9.28; RT2 (min): 14.73; the first peak was the product. Sample Solvent: DCM:ACN=1:1—HPLC; Injection Volume: 2.75 mL; Number of Runs: 1) to afford the crude product. The crude product was purified by reversed-phase flash chromatography with the following conditions: column, 40 g C18 silica gel; mobile phase, MeCN in water (0.1% FA), 25% to 60% gradient in 10 min; detector, UV 254 nm to afford Compound 183 (79.0 mg, 25.10%) as a white solid.

LC-MS-Compound 183: (ES, m/z): [M−HCOOH+H]⁺ 997

H-NMR-Compound 183: (400 MHz, DMSO-d6, ppm): δ 0.78-1.35 (m, 2H), δ 1.37-1.86 (m, 8H), δ 1.91-2.22 (m, 4H), δ 2.31-2.46 (m, 1H), δ 2.51-2.68 (m, 1H), δ 2.75-3.01 (m, 6H), δ 3.15-3.26 (m, 2H), δ 3.35-3.51 (m, 12H), δ 3.71-3.82 (m, 2H), δ 4.21-4.23 (m, 2H), δ 4.30-4.37 (m, 1H), δ 5.02-5.10 (m, 1H), δ 5.19-5.22 (m, 2H), δ 7.00-7.11 (m, 3H), δ 7.14-7.18 (m, 2H), δ 7.23-7.26 (m, 2H), δ 7.38-7.47 (m, 1H), δ 7.53-7.56 (d, 1H), δ 7.63-7.67 (m, 1H), δ 7.84-8.06 (m, 3H), δ 8.35 (s, 1H), δ 10.98 (s, 1H).

### Example 182. Compound 184

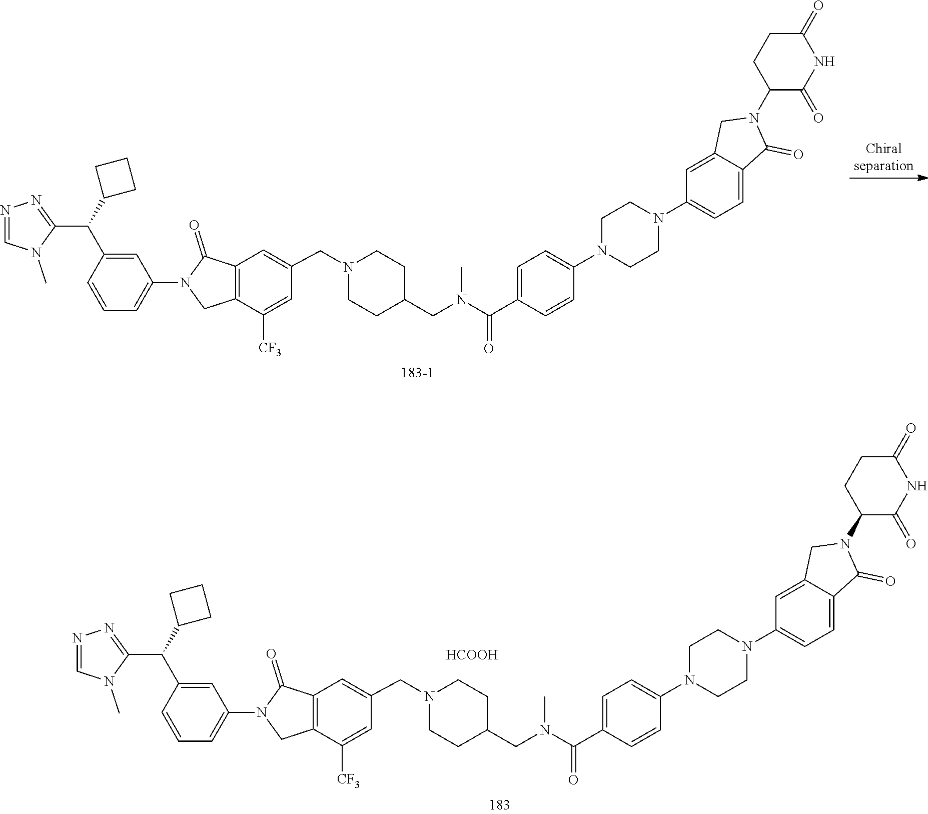 [SMILES: CN(CC1CCN(Cc2cc3c(c(C(F)(F)F)c2)CN(c2cccc([C@H](c4nncn4C)C4CCC4)c2)C3=O)CC1)C(=O)c1ccc(N2CCN(c3ccc4c(c3)CN(C3CCC(=O)NC3=O)C4=O)CC2)cc1 ; CN(CC1CCN(Cc2cc3c(c(C(F)(F)F)c2)CN(c2cccc([C@H](c4nncn4C)C4CCC4)c2)C3=O)CC1)C(=O)c1ccc(N2CCN(c3ccc4c(c3)CN([C@H]3CCC(=O)NC3=O)C4=O)CC2)cc1 ; "HCOOH"]

### Synthesis of Compound 184.

The Compound 183-1 (300 mg) was purified by Prep-Chiral-HPLC with the following conditions (Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 90% B to 90% B in 25 min; Wave Length: 220/254 nm; RT1 (min): 9.28; RT2 (min): 14.73; the second product was the product. Sample Solvent: DCM:ACN=1:1—HPLC; Injection Volume: 2.75 mL; Number of Runs: 1) to afford the crude product. The crude product was purified by reversed-phase flash chromatography with the following conditions: column, 40 g C18 silica gel; mobile phase, MeCN in water (0.1% FA), 25% to 60% gradient in 10 min; detector, UV 254 nm to afford Compound 184 (59.1 mg, 18.62%) as a white solid.

LC-MS-Compound 184: (ES, m/z): [M−HCOOH+H]⁺ 997

H-NMR-Compound 184: (400 MHz, DMSO-d6, ppm): δ 0.78-1.35 (m, 2H), δ 1.37-1.86 (m, 8H), δ 1.91-2.01 (m, 2H), δ 2.02-2.10 (m, 2H), δ 2.31-2.46 (m, 1H), δ 2.51-2.68 (m, 1H), δ 2.75-3.01 (m, 6H), δ 3.15-3.26 (m, 2H), δ 3.35-3.51 (m, 12H), δ 3.71-3.82 (m, 2H), δ 4.21-4.23 (m, 2H), δ 4.30-4.37 (m, 1H), δ 5.02-5.10 (m, 1H), δ 5.19-5.22 (m, 2H), δ 7.00-7.11 (m, 3H), δ 7.14-7.18 (m, 2H), δ 7.23-7.26 (m, 2H), δ 7.38-7.47 (m, 1H), δ 7.53-7.56 (d, 1H), δ 7.63-7.67 (m, 1H), δ 7.84-8.06 (m, 3H), δ 8.35 (s, 1H), δ 10.98 (s, 1H).

### Example 183. Compound 185

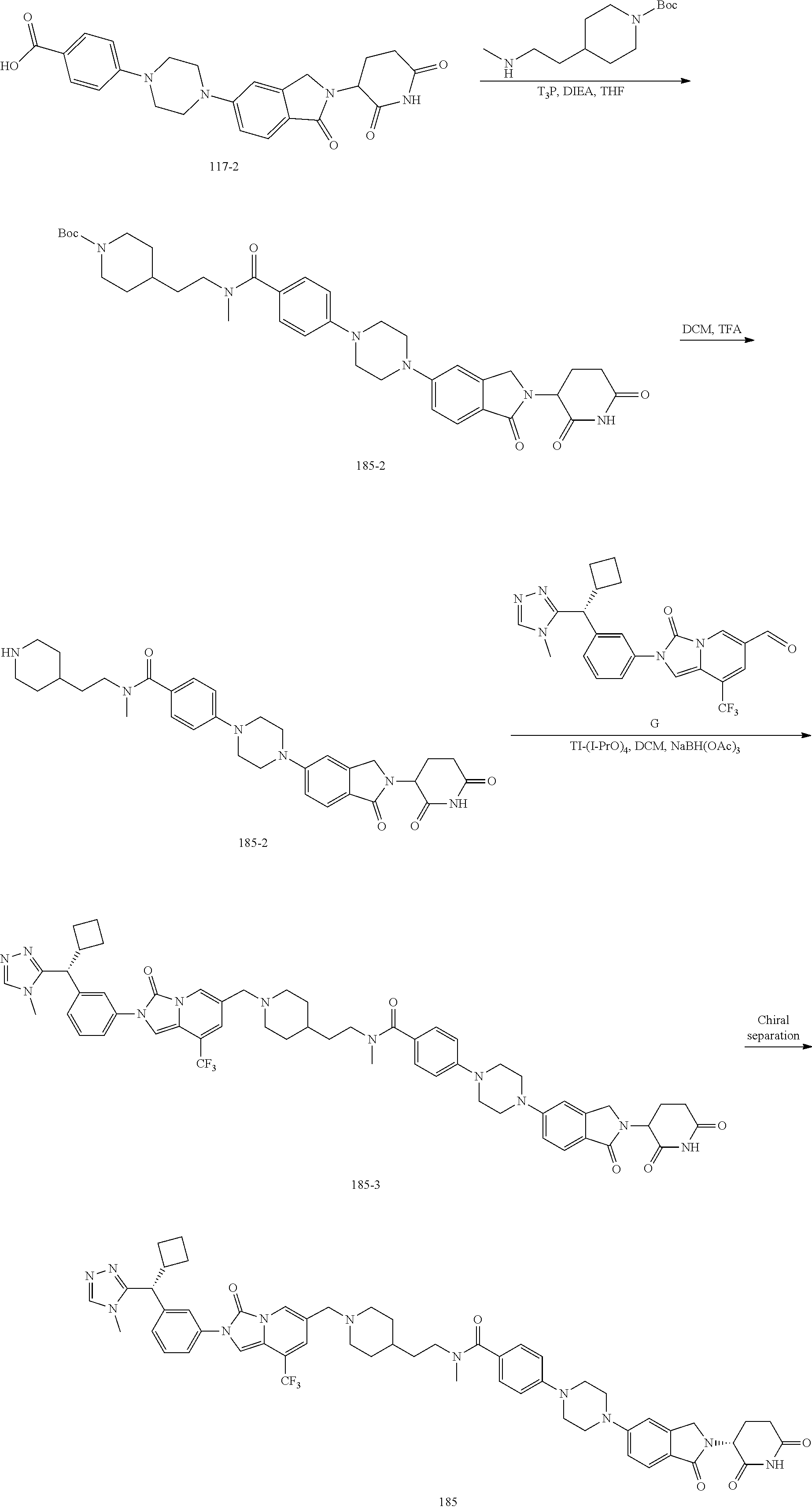

### Synthesis of Compound 185-1.

Into a 100 mL round-bottom flask were added Compound 117-2 (2.5 g, 5.57 mmol, 1 equiv), tert-butyl 4-[2-(methylamino)ethyl]piperidine-1-carboxylate (1.6 g, 6.69 mmol, 1.2 equiv), T3P (7.0 g, 11.15 mmol, 2.0 equiv, 50% in EtOAc), DIEA (1.4 g, 11.15 mmol, 2.0 equiv) and THF (30 mL) at room temperature. The resulting mixture was stirred for overnight at 70° C. The resulting mixture was diluted with H₂O/HOAc=10:1 (100 mL). The aqueous layer was extracted with CH₂Cl₂ (3×50 mL). The resulting mixture was concentrated under vacuum. The residue was purified by silica gel column chromatography, eluted with CH₂Cl₂MeOH=40:1 to afford Compound 185-1 (2.5 g, 63%) as a brown solid.

### Synthesis of Compound 185-2.

Into a 100 mL round-bottom flask were added Compound 185-1 (2.5 g, 3.72 mmol, 1.0 equiv) and DCM (23 mL) at room temperature. To the above mixture was added TFA (7 mL) at room temperature. The resulting mixture was stirred for 2 h at room temperature. The resulting mixture was concentrated under vacuum. The residue was purified by reversed-phase flash chromatography with the following conditions: column, C18 silica gel; mobile phase, MeCN in water (0.1% TFA), 0% to 100% gradient in 30 min; detector, UV 220 nm. This resulted in Compound 185-2 (1.5 g, 66%) as a brown solid.

### Synthesis of Compound 185-3.

To a stirred solution of Compound 185-2 (600 mg, 1.05 mmol, 1.0 equiv) and Intermediate G (620 mg, 1.36 mmol, 1.3 equiv) in DCM (6 mL) were added Ti(Oi-Pr)₄ (1191 mg, 4.19 mmol, 4.0 equiv) at room temperature under nitrogen atmosphere. The resulting mixture was stirred for overnight at room temperature under nitrogen atmosphere. To the above mixture was added NaBH(OAc)₃ (666 mg, 3.14 mmol, 3.0 equiv) at room temperature. The resulting mixture was stirred for additional 4 h at room temperature. The reaction was quenched by the addition of water/HOAc=10:1 (80 mL) at room temperature. The aqueous layer was extracted with CH₂Cl₂MeOH (3×50 mL). The resulting mixture was concentrated under reduced pressure. The residue was purified by reversed-phase flash chromatography with the following conditions: column, C18 silica gel; mobile phase, MeCN in water (0.1% FA), 10% to 50% gradient in 30 min; detector, UV 254 nm. The crude product (300 mg) was purified by Prep-Achiral-SFC with the following conditions (Column: YMC-Actus Triart Diol-HILIC, 3*25 cm, 5 μm; Mobile Phase A: CO₂, Mobile Phase B: MeOH (0.1% FA); Flow rate: 75 mL/min; Gradient: isocratic 15% B; Column Temperature (° C.) 35; Back Pressure (bar): 100; Wave Length: 254 nm; RT1 (min): 6.87) to afford Compound 185-3 (130 mg, 11%) as a yellow solid.

### Synthesis of Compound 185.

The Compound 185-3 (130 mg, 0.13 mmol, 1.0 equiv) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 90% B to 90% B in 30 min; Wave Length: 220/254 nm; RT1 (min): 10.02; RT2 (min): 23.92; the first peak was product) to afford crude product. The residue was purified by reversed-phase flash chromatography with the following conditions: column, C18 silica gel; mobile phase, MeCN in water (0.1% FA), 10% to 50% gradient in 25 min; detector, UV 254 nm. This resulted in Compound 185 (27 mg, 20%) as a yellow solid.

LCMS-Compound 185: (ES, m/z): [M+H]⁺ 1012

NMR-Compound 185: (400 MHz, DMSO-d6, δ ppm): 1.12-1.68 (m, 7H), 1.69-1.98 (m, 9H), 1.99-2.09 (m, 1H), 2.31-2.45 (m, 1H), 2.51-2.60 (m, 1H), 2.77-2.81 (m, 2H), 2.82-2.93 (m, 4H), 3.15-3.26 (m, 4H), 3.32-3.38 (m, 4H), 3.42-3.44 (m, 3H), 3.44-3.45 (m, 4H), 4.20-4.36 (m, 3H), 5.03-5.08 (m, 1H), 6.99-7.02 (m, 3H), 7.11-7.18 (m, 2H), 7.19-7.27 (m, 1H), 7.28-7.30 (m, 3H), 7.41-7.53 (m, 1H), 7.55-7.64 (d, 1H), 7.67-7.73 (m, 3H), 8.32 (s, 1H), 10.95 (s, 1H).

### Example 184. Compound 186

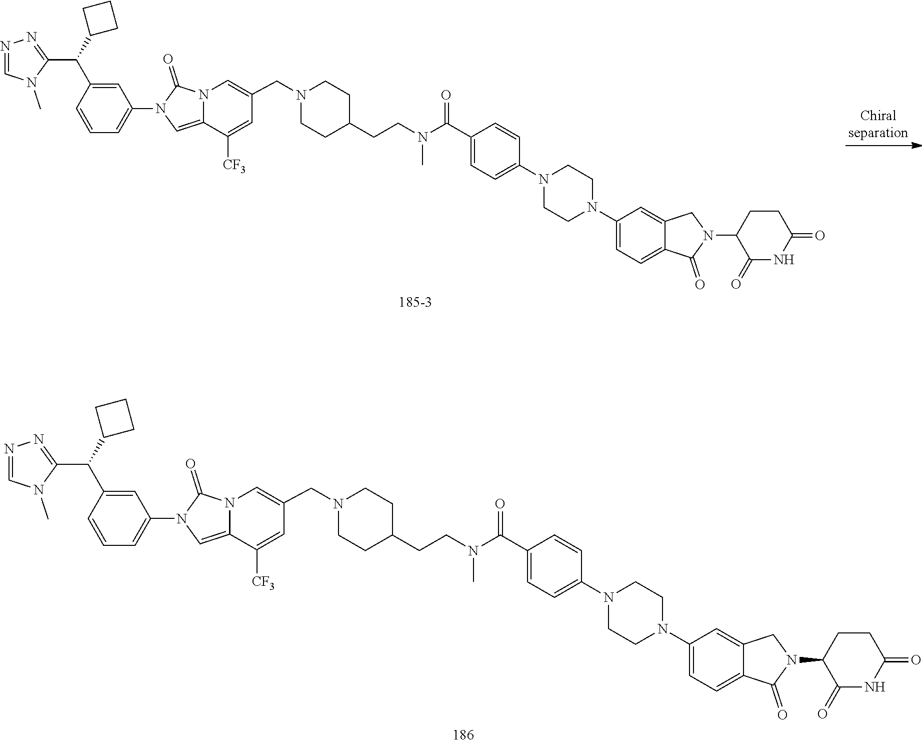 [SMILES: CN(CCC1CCN(Cc2cc(C(F)(F)F)c3cn(-c4cccc([C@H](c5nncn5C)C5CCC5)c4)c(=O)n3c2)CC1)C(=O)c1ccc(N2CCN(c3ccc4c(c3)CN(C3CCC(=O)NC3=O)C4=O)CC2)cc1 ; CN(CCC1CCN(Cc2cc(C(F)(F)F)c3cn(-c4cccc([C@H](c5nncn5C)C5CCC5)c4)c(=O)n3c2)CC1)C(=O)c1ccc(N2CCN(c3ccc4c(c3)CN([C@H]3CCC(=O)NC3=O)C4=O)CC2)cc1]

### Synthesis of Compound 186.

The Compound 185-3 (130 mg, 0.13 mmol, 1.0 equiv) was purified by Prep-Chiral-HPLC with the following conditions (Column: CHIRALPAK IH, 2*25 cm, 5 μm, Mobile Phase A: MTBE (0.1% TEA), Mobile Phase B: DCM:ACN=1:1; Flow rate: 20 mL/min; Gradient: 90% B to 90% B in 30 min; Wave Length: 220/254 min; RT1 (min): 10.02; RT2 (min): 23.92; the second peak was product) to afford crude product. The residue was purified by reversed-phase flash chromatography with the following conditions: column, C18 silica gel; mobile phase, MeCN in water (0.1% FA), 10% to 50% gradient in 25 min; detector, UV 254 nm. This resulted in Compound 186 (25.1 mg, 19%) as a yellow solid.

LCMS-Compound 186: (ES, m/z): [M+H]⁺ 1012

NMR-Compound 186: (400 MHz, DMSO-d6, δ ppm): 1.12-1.68 (m, 7H), 1.69-1.98 (m, 9H), 1.99-2.14 (m, 1H), 2.34-2.41 (m, 1H), 2.55-2.61 (m, 1H), 2.78-2.81 (m, 2H), 2.82-2.95 (m, 4H), 3.13-3.33 (m, 4H), 3.37-3.38 (m, 4H), 3.42-3.44 (m, 3H), 3.45-3.46 (m, 4H), 4.20-4.36 (m, 3H), 5.03-5.08 (m, 1H), 6.99-7.00 (m, 3H), 7.02-7.11 (m, 2H), 7.13-7.20 (m, 1H), 7.27-7.30 (m, 3H), 7.41-7.53 (m, 1H), 7.55-7.64 (d, 1H), 7.67-7.73 (m, 3H), 8.32 (s, 1H), 10.98 (s, 1H).

### Example 185. Compound 187

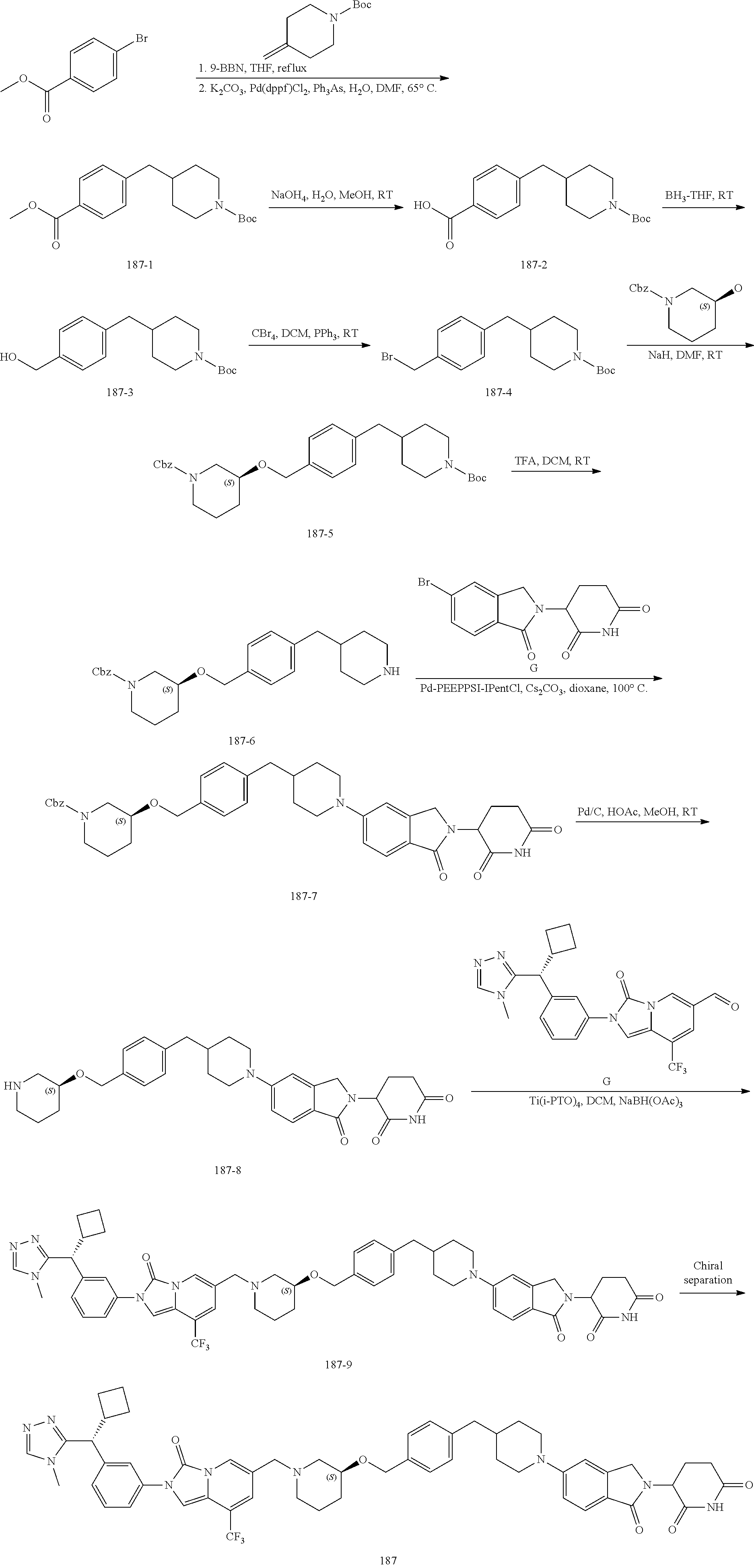

### Synthesis of Compound 187-1.

To a stirred mixture of tert-butyl 4-methylidenepiperidine-1-carboxylate (44.03 g, 223.208 mmol, 1.5 equiv) in THF (400 mL) was added 9-borabicyclo[3.3.1]nonane (297 mL, 297.610 mmol, 2 equiv, 1M) at room temperature under nitrogen atmosphere. The resulting mixture was stirred for 4 h at 65° C. under nitrogen atmosphere. To the above mixture was added methyl 4-bromobenzoate (32 g, 148.805 mmol, 1 equiv), AsPh₃ (9.11 g, 29.761 mmol, 0.2 equiv) and Pd(dppf)Cl₂ (10.89 g, 14.881 mmol, 0.1 equiv) in DMF (300 mL)/H₂O (15 mL) at room temperature. The resulting mixture was stirred overnight at 65° C. The mixture was allowed to cool down to room temperature. The reaction was quenched with NH₄Cl (aq.) (500 mL) at room temperature. The aqueous layer was extracted with EtOAc (3×300 mL). The resulting mixture was concentrated under vacuum. The residue was purified by silica gel column chromatography, eluted with PE/EA (5:1) to afford Compound 187-1 (20 g, 40.31%) as a yellow solid.

### Synthesis of Compound 187-2.

To a stirred mixture of Compound 187-1 (20 g, 59.983 mmol, 1 equiv) in MeOH (100 mL) was added NaOH(aq.)(1M) (120 mL) at room temperature. The resulting mixture was stirred for 2 h at room temperature. The resulting mixture was concentrated under vacuum. The mixture was acidified to pH 2 with HCl (aq.). The precipitated solids were collected by filtration and washed with water (2×30 mL). This resulted in Compound 187-2 (16 g, 83.51%) as a white solid.

### Synthesis of Compound 187-3.

To a stirred mixture of Compound 187-2 (16 g, 50.094 mmol, 1 equiv) in THF (150 mL) was added BH₃-THF (100 mL, 100.188 mmol, 2 equiv) (1M) at 0° C. The resulting mixture was stirred for 2 h at 0° C. under nitrogen atmosphere. The reaction was quenched with MeOH at 0° C. The resulting mixture was concentrated under vacuum. The residue was purified by silica gel column chromatography, eluted with PE/EA (1:1) to afford Compound 187-3 (13 g, 84.97%) as an off-white solid.

### Synthesis of Compound 187-4.

To a stirred mixture of Compound 187-3 (12.5 g, 40.928 mmol, 1 equiv) and CBr₄ (20.36 g, 61.392 mmol, 1.5 equiv) in DCM (150 mL) was added PPh₃ (16.10 g, 61.392 mmol, 1.5 equiv) at room temperature. The resulting mixture was stirred for 6 h at room temperature. The reaction was quenched with water (100 mL) at room temperature. The aqueous layer was extracted with CH₂Cl₂ (3×100 mL). The resulting mixture was concentrated under vacuum. The residue was purified by silica gel column chromatography, eluted with PE/EA (5:1) to afford Compound 187-4 (15 g, 99.51%) as a brown solid.

### Synthesis of Compound 187-5.

To a stirred mixture of NaH (3.15 g, 78.736 mmol, 2 equiv, 60%) in DMF (150 mL) was added benzyl (3S)-3-hydroxypiperidine-1-carboxylate (11.12 g, 47.242 mmol, 1.2 equiv) at ° 0 C under nitrogen atmosphere. To the above mixture was added Compound 187-4 (14.5 g, 39.368 mmol, 1 equiv) at 0° C. The resulting mixture was stirred overnight at room temperature. The reaction was quenched with NH₄Cl (aq.) (300 mL) at room temperature. The aqueous layer was extracted with EtOAc (3×200 mL). The resulting mixture was concentrated under vacuum. The residue was purified by silica gel column chromatography, eluted with PE/EA (1:1) to afford Compound 187-5 (15 g, 72.90%) as a white solid.

### Synthesis of Compound 187-6.

To a stirred mixture of Compound 187-5 (15 g, 28.698 mmol, 1 equiv) in DCM (120 mL) was added TFA (30 mL) at room temperature. The resulting mixture was stirred for 1 h at room temperature. The resulting mixture was concentrated under vacuum. The residue was purified by silica gel column chromatography, eluted with CH₂Cl₂/MeOH (50:1) to afford Compound 187-6 (10 g, 82.46%) as a brown solid.

### Synthesis of Compound 187-7.

To a stirred mixture of benzyl Compound 187-6 (10 g, 23.665 mmol, 1 equiv) and 3-(5-bromo-1-oxo-3H-isoindol-2-yl)piperidine-2,6-dione (8.41 g, 26.032 mmol, 1.1 equiv) in dioxane (100 mL) was added Pd-PEPPSI-IPentCl 2-methylpyridine (o-picoline) (1.99 g, 2.366 mmol, 0.1 equiv) and Cs₂CO₃ (23.13 g, 70.995 mmol, 3 equiv) at room temperature. The resulting mixture was stirred overnight at 100° C. The mixture was allowed to cool down to room temperature. The mixture was acidified to pH 6 with HOAc. The resulting mixture was concentrated under vacuum. The residue was purified by silica gel column chromatography, eluted with CH₂Cl₂/MeOH (50:1) to afford benzyl Compound 187-7 (6 g, 38.14%) as a brown solid.

### Synthesis of Compound 187-8.

To a solution of Compound 187-7 (6 g, 9.025 mmol, 1 equiv) and HOAc (3 mL) in MeOH (200 mL) was added Pd/C (10%, 0.6 g) under nitrogen atmosphere in a 500 mL round-bottom flask. The mixture was hydrogenated at room temperature for 2 h under hydrogen atmosphere using a hydrogen balloon, filtered through a Celite pad and concentrated under reduced pressure. This resulted in Compound 187-8 (4 g, 83.52%) as a light yellow oil.

### Synthesis of Compound 187-9.

To a stirred solution of Compound 187-8 (1 g, 1.884 mmol, 1 equiv) and Intermediate G (1.12 g, 2.449 mmol, 1.3 equiv) in DCM (20 mL) was added Ti(i-PrO)₄ (1.07 g, 3.768 mmol, 2 equiv) at room temperature. The resulting mixture was stirred overnight at room temperature. To the above mixture was added NaBH(OAc)₃ (0.80 g, 3.768 mmol, 2 equiv) at room temperature. The resulting mixture was stirred for additional 4 h at room temperature. The reaction was quenched with 10% AcOH (aq.) (200 mL) at room temperature. The resulting mixture was filtered, the filter cake was washed with water 10% AcOH (aq.) (2×50 mL). The aqueous layer was extracted with EtOAc (100 mL). The aqueous layer was concentrated under vacuum. The residue was purified by reversed-phase flash chromatography with the following conditions: column, C18 silica gel; mobile phase, MeCN in Water (0.1% FA), 5% to 35% gradient in 30 min; detector, UV 254 nm. This resulted in Compound 187-9 (400 mg, 21.88%) as a yellow solid.

### Synthesis of Compound 187.

The Compound 187-9 (400 mg) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 80% B to 80% B in 8.5 min; Wave Length: 220/254 nm; RT1 (min): 4.68; RT2 (min): 6.70, the first peak is product) to afford the crude product. The crude product was purified by reversed-phase flash chromatography with the following conditions: column, C18 silica gel; mobile phase, MeCN in water (0.1% FA), 5% to 35% gradient in 20 min; detector, UV 254 nm. This resulted in Compound 187 (105.0 mg, 26.25%) as a yellow solid.

LC-MS-Compound 187: (ES, m/z): [M+H]⁺ 970

H-NMR-Compound 187: (400 MHz, DMSO-d6, δ ppm): 1.20-1.32 (m, 2H), 1.34-1.38 (m, 1H), 1.58-1.61 (m, 2H), 1.78-1.80 (m, 7H), 1.92-2.05 (m, 3H), 2.06-2.18 (m, 2H), 2.37-2.49 (m, 1H), 2.50-2.51 (m, 1H), 2.60-2.63 (m, 2H), 2.73-2.76 (m, 2H), 2.86-2.88 (m, 2H), 3.14-3.27 (m, 1H), 3.30-3.39 (m, 4H), 3.42-3.44 (m, 4H), 3.81-3.84 (m, 2H), 4.15-4.32 (m, 3H) 4.44-4.46 (m, 2H), 5.02-5.04 (d, 1H), 7.00-7.04 (m, 3H), 7.10-7.12 (m, 2H), 7.17-7.21 (m, 3H), 7.32 (s, 1H), 7.41-7.49 (m, 2H), 7.65-7.75 (m, 3H), 8.31 (s, 1H), 10.97 (s, 1H).

### Example 186. Compound 188

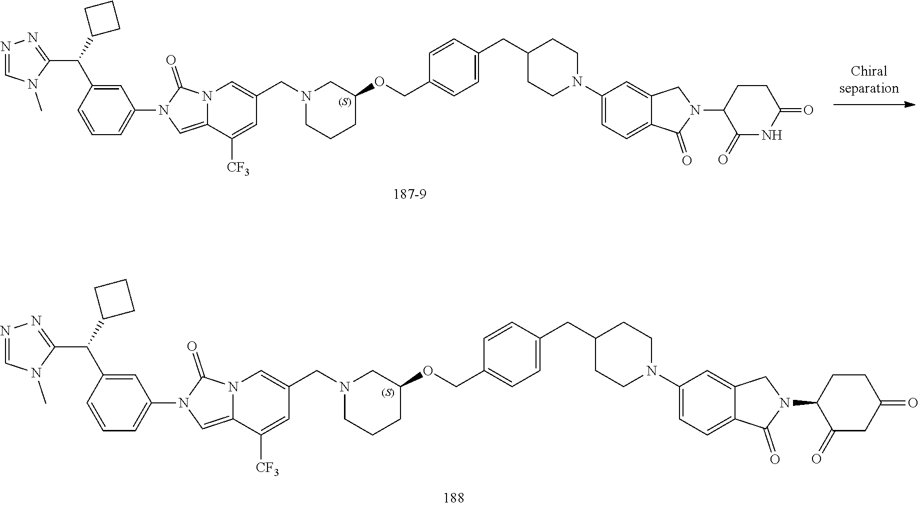

### Synthesis of Compound 188.

The Compound 187-9 (400 mg) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 80% B to 80% B in 8.5 min; Wave Length: 220/254 nm; RT1 (min): 4.68; RT2 (min): 6.70, the second peak is product) to afford the crude product. The crude product was purified by Prep-HPLC with the following conditions (Column. Sunfire prep C18 column, 30*150 mm, 5 μm, Mobile Phase A: Water (0.1% FA), Mobile Phase B: ACN; Flow rate: 60 mL/min; Gradient: 27% B to 38% B in 7 min, Wave Length: 254; 220 nm; RT1 (min): 6.37) to afford Compound 188 (55.0 mg, 13.7%) as a yellow solid

LC-MS-Compound 188: (ES, m/z): [M+H]⁺ 970

H-NMR-Compound 188: (400 MHz, DMSO-d₆, δ ppm): 1.20-1.32 (m, 2H), 1.34-1.38 (m, 1H), 1.58-1.61 (m, 2H), 1.78-1.80 (m, 7H), 1.92-2.18 (m, 5H), 2.37-2.49 (m, 1H), 2.50-2.51 (m, 1H), 2.60-2.63 (m, 2H), 2.73-2.76 (m, 2H), 2.86-2.88 (m, 2H), 3.14-3.27 (m, 1H), 3.30-3.39 (m, 4H), 3.42-3.44 (m, 4H), 3.81-3.84 (m, 2H), 4.15-4.32 (m, 3H) 4.44-4.46 (m, 2H), 5.02-5.04 (d, 1H), 7.00-7.04 (m, 3H), 7.10-7.12 (m, 2H), 7.17-7.21 (m, 3H), 7.32 (s, 1H), 7.41-7.49 (m, 2H), 7.65-7.75 (m, 3H), 8.31 (s, 1H), 10.97 (s, 1H).

### Example 187. Compound 189

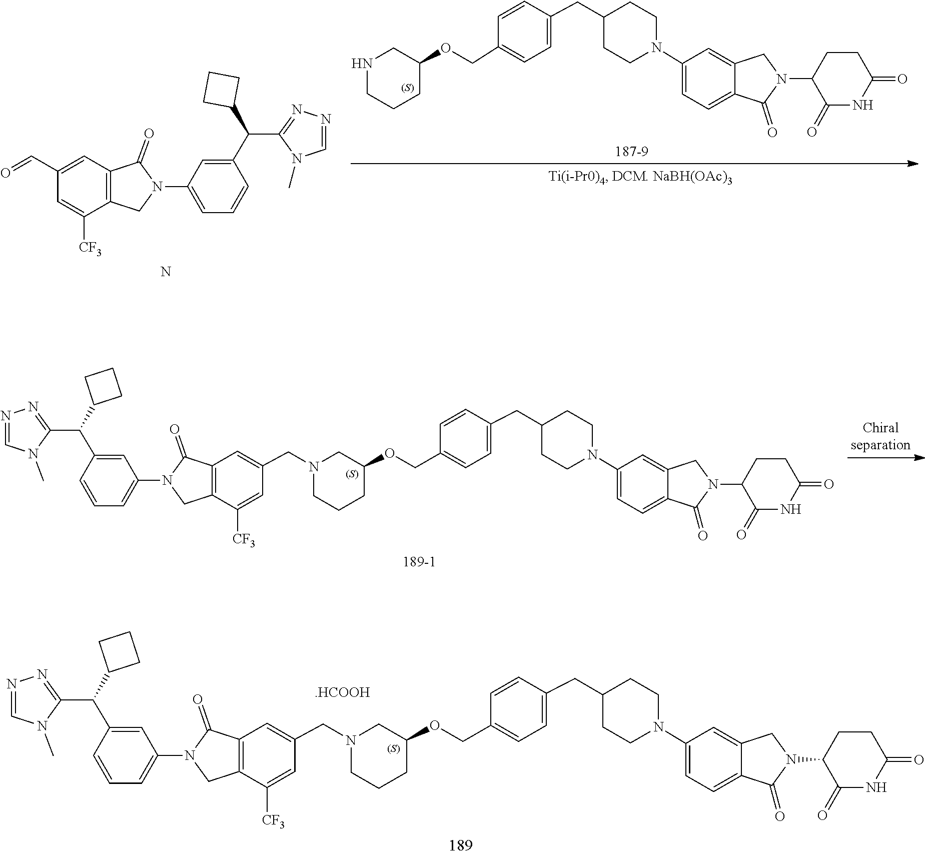

### Synthesis of Compound 189-1.

To a stirred solution of Intermediate N (800 mg, 1.760 mmol, 1.3 equiv) and Compound 187-9 (718.59 mg, 1.354 mmol, 1 equiv) in DCM (20 mL) was added Ti(i-PrO)₄ (769.72 mg, 2.708 mmol, 2 equiv) at room temperature. The resulting mixture was stirred overnight at room temperature. To the above mixture was added NaBH(OAc)₃ (573.99 mg, 2.708 mmol, 2 equiv) at room temperature. The resulting mixture was stirred for additional 4 h at room temperature. The reaction was quenched with 10% HOAc (aq.) (200 mL) at room temperature. The resulting mixture was filtered, the filter cake was washed with 10% HOAc (aq.) (2×20 mL). The aqueous layer was extracted with EtOAc (100 mL). The aqueous layer was concentrated under vacuum. The residue was purified by reversed-phase flash chromatography with the following conditions: column, C18 silica gel; mobile phase, MeCN in water (0.1% FA), 0% to 30% gradient in 35 min; detector, UV 254 nm. This resulted in Compound 189-1 (500 mg, 34.22%) as a yellow solid.

### Synthesis of Compound 189-2.

The Compound 189-1 (500 mg) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 90% B to 90% B in 9 min; Wave Length: 220/254 nm; RT1 (min): 4.95; RT2 (min): 7.13, the first peak is product) to afford the crude product. The crude product was purified by reversed-phase flash chromatography with the following conditions: column, C18 silica gel; mobile phase, MeCN in water (0.1% FA), 0% to 28% gradient in 20 min; detector, UV 254 nm. This resulted in Compound 187 (113.4 mg, 22.68%) as a yellow solid.

LC-MS-Compound 189: (ES, m/z): [M−HCOOH+H]⁺ 969

H-NMR-Compound 189: (400 MHz, DMSO-d₆, δ ppm): 1.12-1.28 (m, 3H), 1.42-1.41 (m, 1H), 1.59-1.68 (m, 9H), 1.92-2.12 (m, 5H), 2.26-2.38 (m, 1H), 2.61-2.70 (m, 2H), 2.71-2.78 (m, 2H), 2.89-2.92 (m, 2H), 3.15-3.25 (m, 2H), 3.46-3.57 (m, 5H), 3.70 (s, 2H), 3.87-3.85 (d, 2H), 4.15-4.32 (m, 3H) 4.44-4.46 (m, 2H), 5.02-5.04 (d, 1H), 5.16 (s, 2H), 6.99-7.04 (m, 3H), 7.11-7.21 (m, 4H), 7.35-7.39 (m, 1H), 7.47-7.49 (d, 1H), 7.75-7.77 (d, 1H), 7.91-7.96 (m, 3H), 8.15 (s, 1H), 8.31 (s, 1H), 10.95 (s, 1H).

### Example 188. Compound 190

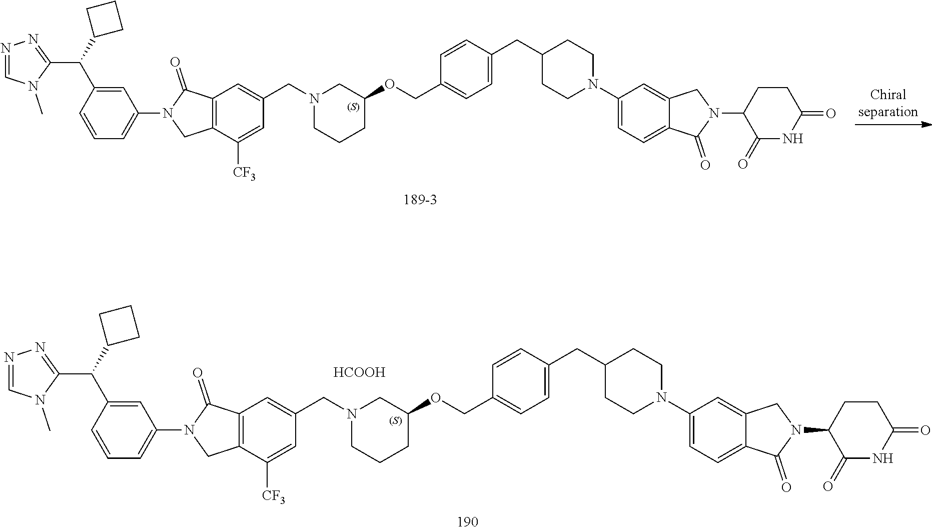

### Synthesis of Compound 190.

The Compound 189-3 (400 mg) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 90% B to 90% B in 9 min; Wave Length: 220/254 nm; RT1 (min): 4.95; RT2 (min): 7.13, the second peak is product) to afford the crude product. The crude product was purified by Prep-HPLC with the following conditions (Column. Sunfire prep C18 column, 30*150 mm, 5 μm, Mobile Phase A: Water (0.1% FA), Mobile Phase B: ACN; Flow rate: 60 mL/min; Gradient: 26% B to 40% B in 7 min, Wave Length: 254; 220 nm; RT1 (min): 6.52) to afford Compound 190 (107.3 mg, 26.82%) as a yellow solid

LC-MS-Compound 190: (ES, m/z): [M−HCOOH+H]⁺ 969

H-NMR-Compound 190: (400 MHz, DMSO-d₆, δ ppm): 1.12-1.28 (m, 3H), 1.42-1.41 (m, 1H), 1.59-1.68 (m, 9H), 1.92-2.12 (m, 5H), 2.26-2.38 (m, 1H), 2.61-2.70 (m, 2H), 2.71-2.78 (m, 2H), 2.89-2.92 (m, 2H), 3.15-3.25 (m, 2H), 3.46-3.57 (m, 5H), 3.70 (s, 2H), 3.87-3.85 (d, 2H), 4.15-4.32 (m, 3H) 4.44-4.46 (m, 2H), 5.02-5.04 (d, 1H), 5.16 (s, 2H), 6.99-7.04 (m, 3H), 7.11-7.21 (m, 4H), 7.35-7.39 (m, 1H), 7.47-7.49 (d, 1H), 7.75-7.78 (d, 1H), 7.91 (s, 2H), 7.96 (s, 1H), 8.15 (s, 1H), 8.31 (s, 1H), 10.95 (s, 1H).

### Example 189. Compound 191

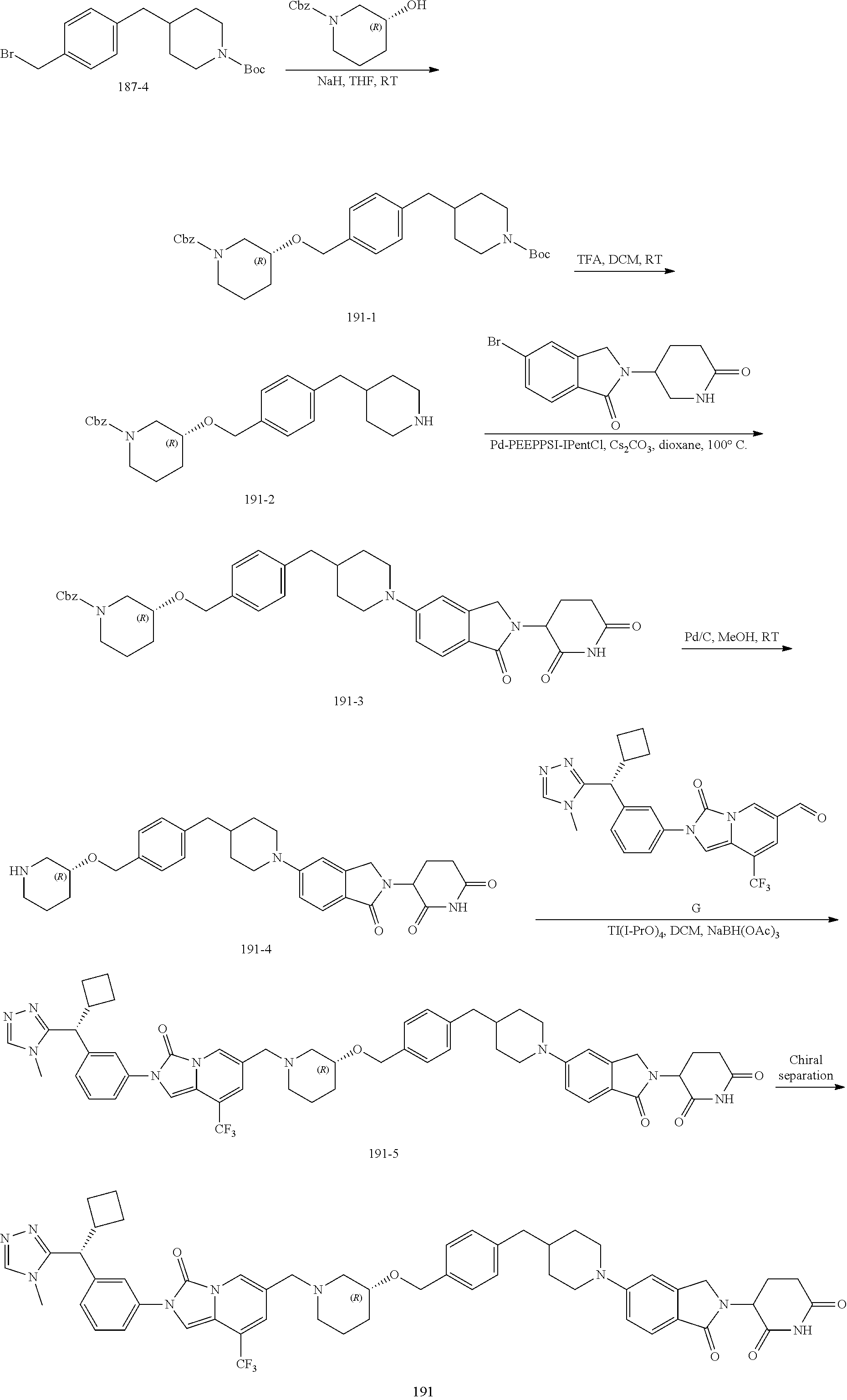

### Synthesis of Compound 191-1.

To a solution of benzyl (R)-3-hydroxypiperidine-1-carboxylate (12.8 g, 54.30 mmol, 1.0 equiv) in THF (200 mL) was added NaH (3.26 g, 81.45 mmol, 1.5 equiv, 60% in oil) at 0 degrees C. The mixture was stirred for 30 min. Compound 187-4 (20.0 g, 54.30 mmol, 1.0 equiv) was added and the mixture was allowed to warm to RT and stirred for overnight. The reaction was quenched with sat. NH₄Cl (aq.) at room temperature. The aqueous layer was extracted with CH₂Cl₂ (3×500 mL). The resulting mixture was concentrated under reduced pressure. The residue was purified by silica gel column chromatography, eluted with CH₂Cl₂/MeOH=50:1 to afford Compound 191-1 (20.0 g, 64%) as a yellow solid.

### Synthesis of Compound 191-2.

Into a 800 mL sealed tube were added Compound 191-1 (20.0 g, 38.26 mmol, 1.0 equiv), DCM (300 mL) and TFA (100 mL) at room temperature. The mixture was stirred for 2 h at room temperature. The reaction was quenched with sat. NaHCO₃ (aq.) at room temperature. The aqueous layer was extracted with CH₂Cl₂ (3×500 mL). The resulting mixture was concentrated under reduced pressure. The resulting product was used in the next step directly without further purification.

### Synthesis of Compound 191-3.

Into a 250 mL sealed tube were added Compound 191-2 (10.0 g, 23.67 mmol, 1.0 equiv), 3-(5-bromo-1-oxo-3H-isoindol-2-yl)piperidine-2,6-dione (9.94 g, 30.76 mmol, 1.3 equiv), Cs₂CO₃ (23.1 g, 70.99 mmol, 3 equiv), dioxane (150 mL) and {1,3-bis[2,6-bis(pentan-3-yl)phenyl]-4,5-dichloro-2,3-dihydro-1H-imidazol-2-yl}dichloro(2-methyl-1lambda4-pyridin-1-yl)palladium (1.0 g, 1.18 mmol, 0.05 equiv) at room temperature. The mixture was stirred for overnight at 100° C. under nitrogen atmosphere. The reaction was quenched with sat. NH₄Cl (aq.) at room temperature. The aqueous layer was extracted with EtOAc (3×200 mL). The resulting mixture was concentrated under reduced pressure. The residue was purified by silica gel column chromatography, eluted with CH₂Cl₂/MeOH (30:1) to afford Compound 191-3 (5.0 g, 29%) as a yellow solid.

### Synthesis of Compound 191-4.

To a solution of Compound 191-3 (4.0 g, 6.02 mmol, 1.0 equiv) in MeOH (120 mL) was added AcOH (180 mg, 3.01 mmol, 0.5 equiv) and Pd/C (640 mg) under nitrogen atmosphere in a 500 mL round-bottom flask. The mixture was hydrogenated at room temperature for 1 h under hydrogen atmosphere using a hydrogen balloon, filtered through a Celite pad and concentrated under reduced pressure. This resulted in Compound 191-4 (3.0 g, 87%) as a white solid.

### Synthesis of Compound 191-5.

To a stirred solution of Compound 191-4 (1.0 g, 1.88 mmol, 1.0 equiv) and Intermediate G (1.3 g, 2.83 mmol, 1.5 equiv) in DCM (10 mL) were added Ti(Oi-Pr)₄ (2.1 g, 7.54 mmol, 4.0 equiv) at room temperature. The resulting mixture was stirred for overnight at room temperature. To the above mixture was added NaBH(OAc)₃ (1.2 g, 5.65 mmol, 3.0 equiv) at room temperature. The resulting mixture was stirred for additional 4 h at room temperature. The reaction was quenched by the addition of water/HOAc=10:1 (100 mL) at room temperature. The aqueous layer was extracted with CH₂Cl₂/MeOH=5:1 (3×80 mL). The resulting mixture was concentrated under reduced pressure. The residue was purified by reversed-phase flash chromatography with the following conditions: column, C18 silica gel; mobile phase, MeCN in water (0.1% TFA), 10% to 50% gradient in 30 min; detector, UV 254 nm. The crude product (350 mg) was purified by Prep-HPLC with the following conditions (Column: XSelect CSH Fluoro Phenyl, 30*150 mm, 5 μm; Mobile Phase A: Water (0.1% FA), Mobile Phase B: ACN; Flow rate: 60 mL/min; Gradient: 26% B to 37% B in 8 min, 37% B; Wave Length: 254; 220 nm; RT1 (min): 7.4) to Compound 191-5 (140 mg, 7%) as a yellow solid.

### Synthesis of Compound 191.

Compound 191-5 (140 mg) was purified by Prep-Chiral-HPLC with the following conditions (Column: (R, R)-WHELK-O1-Kromasi, 5*25 cm, 5 μm; Mobile Phase A: Hex (0.2% TEA), Mobile Phase B: IPA:DCM=1:1; Flow rate: 20 mL/min; Gradient: 80% B to 80% B in 39 min; Wave Length: 220/254 nm; RT1 (min): 19.83; RT2 (min): 30.71; the first peak was product) to afford crude product. The residue was purified by reversed-phase flash chromatography with the following conditions: column, C18 silica gel; mobile phase, MeCN in water (0.1% FA), 10% to 50% gradient in 25 min; detector, UV 254 nm. This resulted in Compound 191 (28 mg, 20%) as a yellow solid.

LCMS-Compound 191: (ES, m/z): [M+H]⁺ 970

NMR-Compound 191: (400 MHz, DMSO-d6, δ ppm): 1.19-1.29 (m, 3H), 1.30-1.51 (m, 1H), 1.58-1.95 (m, 9H), 1.96-2.21 (m, 5H), 2.32-2.45 (m, 1H), 2.47-2.49 (m, 1H), 2.50-2.60 (m, 2H), 2.73-2.85 (m, 2H), 2.86-2.94 (m, 2H), 3.18-3.29 (m, 1H), 3.30-3.33 (m, 3H), 3.34-3.42 (m, 4H), 3.82-3.85 (m, 2H), 4.16-4.32 (m, 3H), 4.45-4.46 (m, 2H), 5.02-5.05 (m, 1H), 7.00-7.01 (d, 3H), 7.10-7.18 (m, 2H), 7.20-7.22 (m, 3H), 7.32 (s, 1H), 7.41-7.49 (m, 2H), 7.65-7.75 (m, 3H), 8.32 (s, 1H), 11.96 (s, 1H).

### Example 190. Compound 192

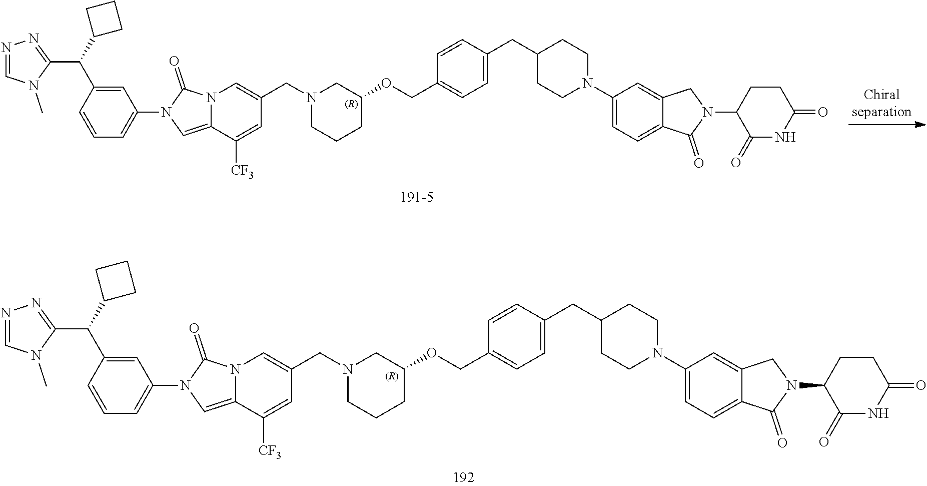

### Synthesis of Compound 192.

The Compound 191-5 (140 mg, 0.14 mmol, 1.0 equiv) was purified by Prep-Chiral-HPLC with the following conditions (Column: (R, R)-WHELK-O1-Kromasi, 5*25 cm, 5 μm; Mobile Phase A: Hex (0.2% TEA), Mobile Phase B: IPA:DCM=1:1; Flow rate: 20 mL/min; Gradient: 80% B to 80% B in 39 min; Wave Length: 220/254 nm; RT1 (min): 19.83; RT2 (min): 30.71; Sample Solvent: IPA:DCM=1:1; Injection Volume: 0.8 mL; the second peak was product) to afford crude product. The residue was purified by reversed-phase flash chromatography with the following conditions: column, (C18 silica gel; mobile phase, MeCN in water (0.1% FA), 10% to 50% gradient in 25 min; detector, UV 254 nm. This resulted in Compound 192 (27 mg, 19%) as a yellow solid.

LCMS-Compound 192: (ES, m/z): [M+H]⁺ 970

NMR-Compound 192: (400 MHz, DMSO-d6, δ ppm): 1.19-1.29 (m, 3H), 1.30-1.51 (m, 1H), 1.58-1.95 (m, 9H), 1.96-2.21 (m, 5H), 2.32-2.45 (m, 1H), 2.47-2.49 (m, 1H), 2.50-2.60 (m, 2H), 2.73-2.85 (m, 2H), 2.86-2.94 (m, 2H), 3.18-3.29 (m, 1H), 3.30-3.33 (m, 3H), 3.34-3.42 (m, 4H), 3.82-3.85 (m, 2H), 4.16-4.33 (m, 3H), 4.45-4.46 (m, 2H), 5.02-5.05 (m, 1H), 7.00-7.01 (d, 3H), 7.10-7.18 (m, 2H), 7.20-7.22 (m, 3H), 7.32 (s, 1H), 7.41-7.49 (m, 2H), 7.65-7.75 (m, 3H), 8.32 (s, 1H), 11.95 (s, 1H).

### Example 191. Compound 193

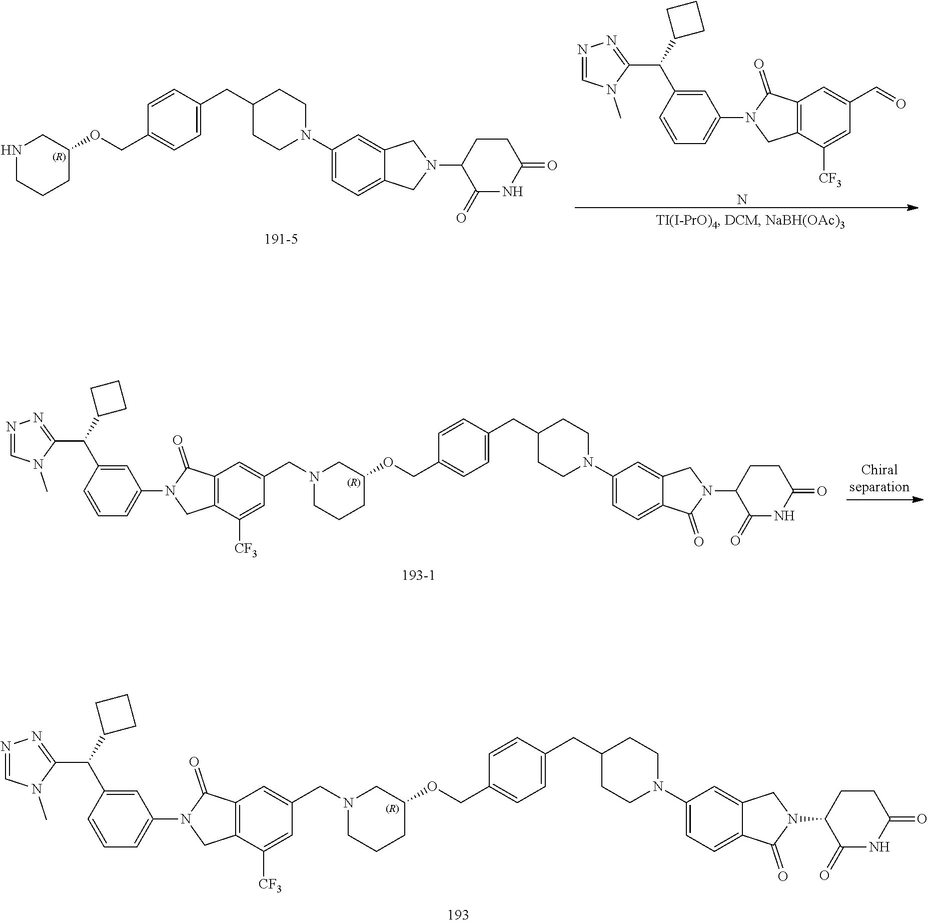

### Synthesis of Compound 193-1.

To a stirred solution of Compound 191-5 (1.0 g, 1.88 mmol, 1.0 equiv) and Intermediate N (860 mg, 1.88 mmol, 1.0 equiv) in DCM (10 mL) was added Ti(Oi-Pr)₄ (2.1 g, 7.54 mmol, 4.0 equiv) at room temperature under nitrogen atmosphere. The resulting mixture was stirred for overnight at room temperature under nitrogen atmosphere. To the above mixture was added NaBH(OAc)₃ (1.2 g, 5.65 mmol, 3.0 equiv) at room temperature. The resulting mixture was stirred for additional 4 h at room temperature. The reaction was quenched by the addition of water/AcOH=10:1 (100 mL) at room temperature. The aqueous layer was extracted with CH₂Cl₂/MeOH=5:1 (3×100 mL). The resulting mixture was concentrated under reduced pressure. The residue was purified by reversed-phase flash chromatography with the following conditions: column, C18 silica gel; mobile phase, MeCN in Water (0.1% FA), 10% to 50% gradient in 30 min; detector, UV 254 nm. This resulted in Compound 193-1 (460 mg, 24%) as a yellow solid.

### Synthesis of Compound 193.

Compound 193-1 (460 mg) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 75% B to 75% B in 13 min; Wave Length: 220/254 nm; RT1 (min): 6.31; RT2 (min): 9.44; the first peak was product) to afford crude product (190 mg). The crude product (190 mg) was purified by reversed-phase flash chromatography with the following conditions: column, C18 silica gel; mobile phase, MeCN in water (0.1% FA), 10% to 50% gradient in 20 min; detector, UV 254 nm. This resulted in Compound 193 (127 mg, 26%) as a white solid.

LCMS-Compound 193: (ES, m/z): [M/2+H]⁺ 485; [M+H]⁺ 969

NMR-Compound 193: (400 MHz, DMSO-d6, δ ppm): 1.21-1.24 (m, 3H), 1.32-1.51 (m, 1H), 1.60-2.08 (m, 14H), 2.32-2.50 (m, 1H), 2.60-2.74 (m, 2H), 2.71-2.77 (m, 2H), 2.86-2.89 (m, 2H), 3.23-3.30 (m, 2H), 3.33-3.42 (m, 3H), 3.43-3.46 (m, 1H), 3.71 (s, 2H), 3.82-3.85 (m, 2H), 4.16-4.32 (m, 3H), 4.44-4.45 (m, 2H), 5.03-5.05 (m, 1H), 5.16 (s, 2H), 6.99-7.11 (m, 3H), 7.13-7.19 (t, 2H), 7.21-7.35 (m, 2H), 7.37-7.39 (t, 1H), 7.47-7.49 (d, 1H), 7.76-7.78 (d, 1H), 7.91 (s, 2H), 7.97 (s, 1H), 8.32 (s, 1H), 10.95 (s, 1H).

### Example 192. Compound 194

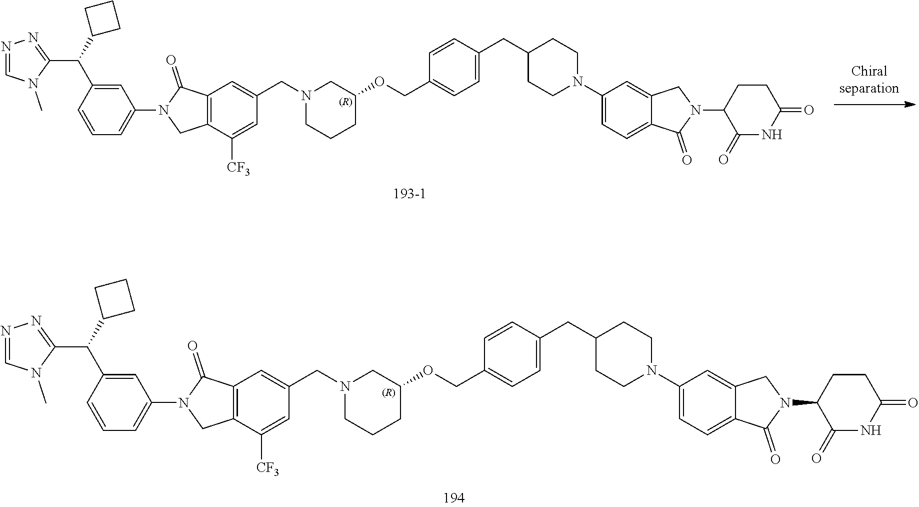

### Synthesis of Compound 194.

The Compound 193-1 (460 mg, 0.47 mmol, 1.0 equiv) was purified by Prep-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 75% B to 75% B in 13 min; Wave Length: 220/254 nm; RT1 (min): 6.31; RT2 (min): 9.44; Sample Solvent: DCM:ACN=1:1; the second peak was product) to afford crude product (190 mg). The crude product was purified by reversed-phase flash chromatography with the following conditions: column, C18 silica gel; mobile phase, MeCN in water (0.1% FA), 10% to 50% gradient in 20 min; detector, UV 254 nm. This resulted in Compound 194 (140 mg, 30%) as a white solid.

LCMS-Compound 194: (ES, m/z): [M/2+H]⁺ 485; [M+H]⁺ 969

NMR-Compound 194: (400 MHz, DMSO-d6, δ ppm): 1.21-1.23 (m, 4H), 1.24-1.25 (m, 1H), 1.26-1.96 (m, 10H), 2.05-2.08 (m, 5H), 2.32-2.49 (m, 1H), 2.50-2.59 (m, 2H), 2.60-2.77 (m, 2H), 2.86-2.89 (m, 2H), 3.20-3.31 (m, 1H), 3.35-3.3.40 (m, 1H), 3.42 (s, 1H), 3.43-3.44 (m, 1H), 3.71 (s, 2H), 3.82-3.85 (m, 2H), 4.16-4.32 (m, 3H), 4.44-4.45 (m, 2H), 5.03-5.05 (m, 1H), 5.16 (s, 2H), 6.99-7.11 (m, 3H), 7.13-7.19 (t, 2H), 7.21-7.35 (m, 2H), 7.37-7.39 (t, 1H), 7.47-7.49 (d, 1H), 7.76-7.78 (d, 1H), 7.91 (s, 2H), 7.97 (s, 1H), 8.32 (s, 1H), 10.95 (s, 1H).

### Example 193. Compound 195

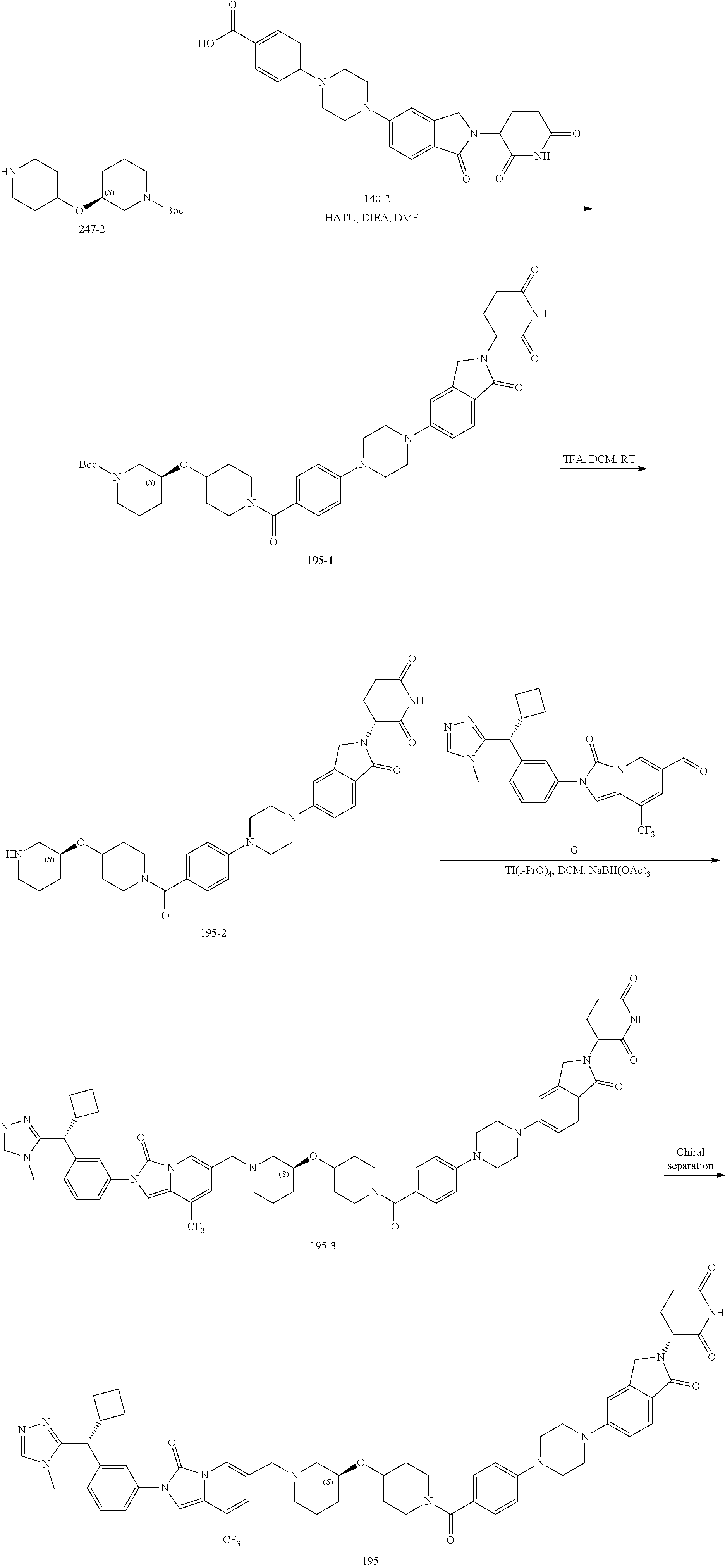

### Synthesis of Compound 195-1.

To a stirred solution of Compound 140-2 (3.47 g, 7.735 mmol, 1.1 equiv) and HATU (6.68 g, 17.580 mmol, 2.5 equiv) in DMF (20 mL) was added DIEA (2.27 g, 17.580 mmol, 2.5 equiv) at room temperature. The resulting mixture was stirred for 1 h at room temperature. To the above mixture was added Compound 247-2 (2 g, 7.032 mmol, 1 equiv) at room temperature. The resulting mixture was stirred for additional overnight at room temperature. The mixture was acidified to pH 5 with water (10% AcOH) (60 mL). The aqueous layer was extracted with EtOAc (3×50 mL). The resulting mixture was concentrated under reduced pressure. The residue was purified by silica gel column chromatography, eluted with DCM/MeOH (20:1) to afford Compound 195-2 (1.2 g, 16.71%) as a brown solid.

### Synthesis of Compound 195-2.

A solution of Compound 195-1 (1.2 g, 1.679 mmol, 1 equiv) and TFA (2 mL) in DCM (12 mL) was stirred for overnight at room temperature. The resulting mixture was concentrated under reduced pressure. The residue was purified by reversed-phase flash chromatography with the following conditions: column, C18 silica gel; mobile phase, MeCN in Water (0.1% FA), 30% to 50% gradient in 20 min; detector, UV 254 nm. This resulted in Compound 195-2 (680 mg, 62.60%) as a brown solid.

### Synthesis of Compound 195-3.

To a stirred solution of Compound 195-2 (537.86 mg, 1.181 mmol, 1.1 equiv) and Intermediate G (537.86 mg, 1.181 mmol, 1.1 equiv) in DCM (8 mL) were added tetrakis(propan-2-yloxy) titanium (915.41 mg, 3.222 mmol, 3 equiv) at room temperature. The resulting mixture was stirred for overnight at room temperature. To the above mixture was added NaBH(OAc)₃ (455.08 mg, 2.148 mmol, 2 equiv) at room temperature. The resulting mixture was stirred for additional 3 h at room temperature. The mixture was acidified to pH 5 with water (10% AcOH) (30 mL). The resulting mixture was filtered; the filter cake was washed with water (10% AcOH) (3×20 mL). The filtrate was concentrated under reduced pressure. The residue was purified by reversed-phase flash chromatography with the following conditions: column, C18 silica gel; mobile phase, MeCN in Water (0.1% FA), 30% to 45% gradient in 20 min; detector, UV 254 nm. This resulted in Compound 195-3 (250 mg, 20.98%) as a yellow solid.

### Synthesis of Compound 195.

The Compound 195-3 (250 mg, 0.237 mmol, 1 equiv) was purified by Chiral Separation with the following conditions (Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 90% B to 90% B in 22 min; Wave Length: 220/254 nm; RT1 (min): 8.95; RT2 (min): 16.87; the first peak was product) to afford crude product (100 mg). The crude product (100 mg) was purified by reversed-phase flash chromatography with the following conditions: column, 40 g C18 silica gel; mobile phase, MeCN in Water (0.1% FA), 30% to 45% gradient in 10 min; detector, UV 254 nm to afford Compound 195-0 (73.0 mg, 29.20%) as a yellow solid.

LC-MS-Compound 195: (ES, m/z): [M+H]⁺ 1054

H-NMR-Compound 195: (400 MHz, DMSO-d6, δ ppm): 1.13-1.28 (m, 1H), 1.39-1.40 (m, 3H), 1.65-1.78 (m, 2H), 1.94-1.96 (m, 7H), 2.32-2.38 (m, 2H), 2.49-2.60 (m, 2H), 2.86-2.87 (m, 2H), 3.17-3.28 (m, 8H), 3.33-3.38 (m, 3H), 3.42-3.45 (m, 2H), 3.67-3.69 (m, 3H), 3.72-3.89 (m, 3H), 4.20-4.37 (m, 3H), 5.07-5.10 (m, 1H), 6.99-7.01 (m, 3H), 7.12-7.18 (m, 3H), 7.20-7.27 (m, 3H), 7.29-7.31 (m, 1H), 7.41-7.45 (m, 1H), 7.54-7.56 (m, 2H), 7.67-7.73 (m, 1H), 8.32 (s, 1H), 10.95 (s, 1H).

### Example 194. Compound 196

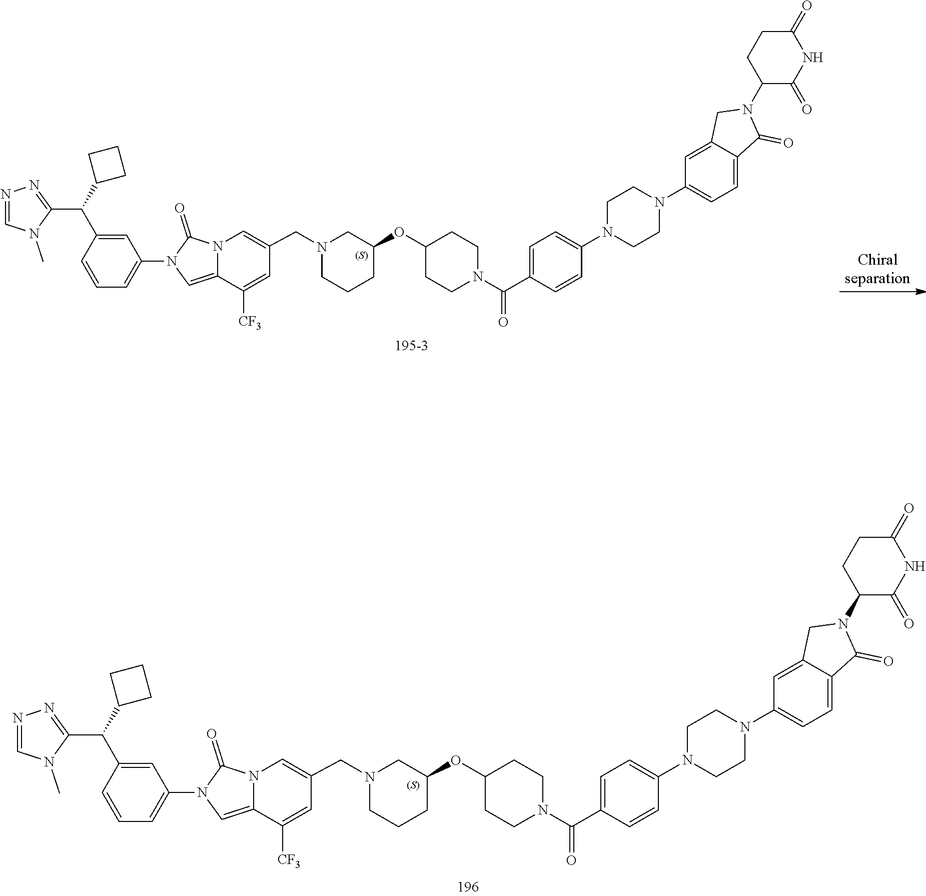

### Synthesis of Compound 196.

The Compound 195-3 (250 mg, 0.237 mmol, 1 equiv) was purified by Chiral Separation with the following conditions (Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 90% B to 90% B in 22 min; Wave Length: 220/254 nm; RT1 (min): 8.95; RT2 (min): 16.87; the second peak was product) to afford crude product (100 mg). The crude product (100 mg) was purified by reversed-phase flash chromatography with the following conditions: column, 40 g C18 silica gel; mobile phase, MeCN in Water (0.1% FA), 30% to 45% gradient in 10 min; detector, UV 254 nm to afford Compound 196-0 (69.8 mg, 27.92%) as a yellow solid.

LC-MS-Compound 196: (ES, m/z): [M+H]⁺ 1054

H-NMR-Compound 196: (400 MHz, DMSO-d6, δ ppm): 1.19-1.22 (m, 1H), 1.27-1.39 (m, 3H), 1.67-1.78 (m, 9H), 1.96-1.98 (m, 3H), 2.03-2.07 (m, 1H), 2.49-2.50 (m, 1H), 2.51-2.67 (m, 3H), 2.86-2.87 (m, 2H), 3.17-3.22 (m, 7H), 3.24-3.25 (m, 2H), 3.33 (s, 3H), 3.38-3.42 (m, 4H), 3.44-3.67 (m, 3H), 4.20-4.37 (m, 3H), 5.03-5.08 (m, 1H), 6.99-7.01 (m, 3H), 7.12-7.19 (m, 3H), 7.27-7.31 (m, 3H), 7.41-7.45 (m, 1H), 7.54-7.56 (m, 1H), 7.67-7.73 (m, 3H), 8.25-8.32 (s, 1H), 10.96 (s, 1H).

### Example 195. Compound 197

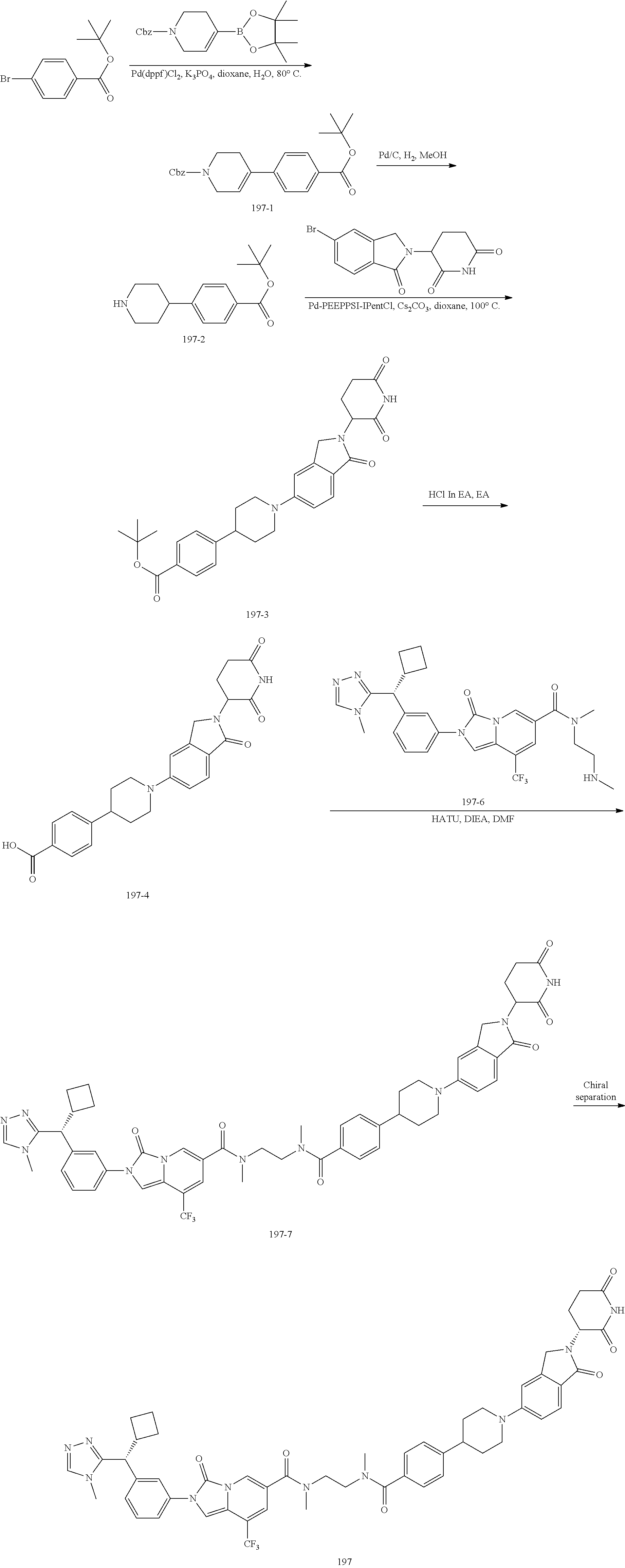

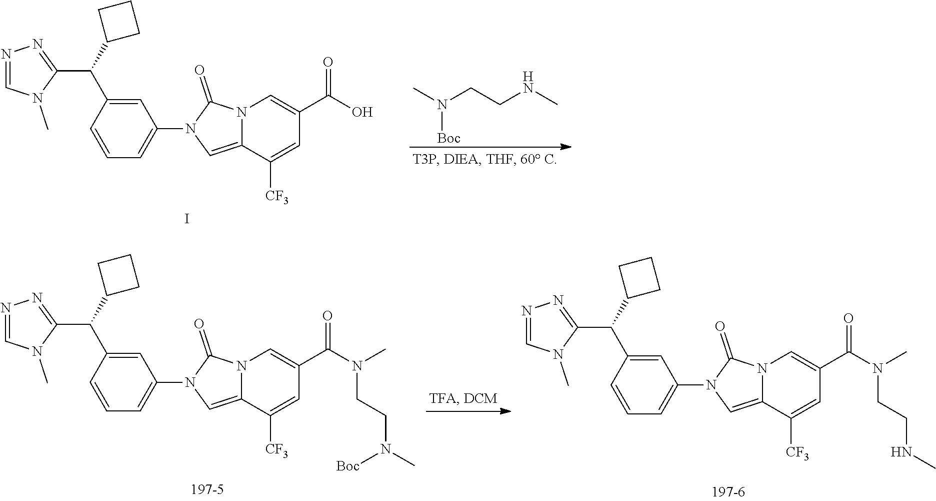

### Synthesis of Compound 197-1.

To a stirred solution of tert-butyl 4-bromobenzoate (20 g, 77.783 mmol, 1 equiv) and benzyl 4-(4,4,5,5-tetramethyl-1,3,2-dioxaborolan-2-yl)-3,6-dihydro-2H-pyridine-1-carboxylate (40.05 g, 116.674 mmol, 1.5 equiv) in dioxane (160 mL) and H₂O (40 mL) were added K₃PO₄ (49.53 g, 233.349 mmol, 3 equiv) and Pd(dppf)Cl₂ (5.69 g, 7.778 mmol, 0.1 equiv) at room temperature under nitrogen atmosphere. The resulting mixture was stirred for overnight at 80° C. under nitrogen atmosphere. The resulting mixture was diluted with water (400 mL). The resulting mixture was extracted with EtOAc (3×500 mL). The combined organic layers were concentrated under reduced pressure. The residue was purified by silica gel column chromatography, eluted with PE/EA (25:1) to afford Compound 197-1 (32 g, 88.87%) as a colorless oil.

### Synthesis of Compound 197-2.

To a solution of Compound 197-1 (23 g, 58.452 mmol, 1 equiv) in MeOH (700 mL) was added Pd/C (4.5 g, 10%) under nitrogen atmosphere in a 2000 mL round-bottom flask. The mixture was hydrogenated at room temperature for 2 h under hydrogen atmosphere using a hydrogen balloon. The resulting mixture was filtered; the filter cake was washed with MeOH (3×200 mL). The filtrate was concentrated under reduced pressure. This resulted in Compound 197-2 (14.1 g, 92.29%) as a light yellow oil.

### Synthesis of Compound 197-3.

To a stirred solution of Compound 197-2 (14 g, 53.565 mmol, 1 equiv) and 3-(5-bromo-1-oxo-3H-isoindol-2-yl) piperidine-2,6-dione (25.96 g, 80.347 mmol, 1.5 equiv) in dioxane (450 mL) were added Cs₂CO₃ (52.36 g, 160.695 mmol, 3 equiv) and {1,3-bis[2,6-bis(pentan-3-yl) phenyl]-4,5-dichloro-2,3-dihydro-1H-imidazol-2-yl} dichloro(2-methyl-1lambda4-pyridin-1-yl) palladium (4.51 g, 5.357 mmol, 0.1 equiv) at room temperature under nitrogen atmosphere. The resulting mixture was stirred for overnight at 100° C. under nitrogen atmosphere. The reaction was quenched by the addition of Acetic acid (10% in water) (1500 mL) at room temperature. The resulting mixture was extracted with CH₂Cl₂/MeOH (10:1) (3×1 L). The combined organic layers were concentrated under reduced pressure. The residue was purified by silica gel column chromatography, eluted with CH₂Cl₂/MeOH (60:1) to afford Compound 197-3 (18 g, 66.73%) as a yellow solid.

### Synthesis of Compound 197-4.

To a stirred solution of Compound 197-3 (14.5 g, 28.793 mmol, 1 equiv) in EA (70 mL) was added HCl(gas) in EA (70 mL, 280 mmol, 10 equiv) dropwise at room temperature. The resulting mixture was stirred for 1 h at room temperature under nitrogen atmosphere. The precipitated solids were collected by filtration and washed with EtOAc (2×30 mL). This resulted in Compound 197-4 (11.6 g, 90.03%) as a white solid.

### Synthesis of Compound 197-5.

To a stirred solution of Intermediate I (2 g, 4.242 mmol, 1 equiv) and DIEA (1.64 g, 12.726 mmol, 3 equiv) in THF (20 mL) were added tert-butyl N-methyl-N-[2-(methylamino)ethyl]carbamate (1.60 g, 8.484 mmol, 2 equiv) and T3P (2.02 g, 6.363 mmol, 1.5 equiv) at room temperature under nitrogen atmosphere. The resulting mixture was stirred for 3 h at 60° C. The reaction was quenched by the addition of water (60 mL) at room temperature. The aqueous layer was extracted with EtOAc (3×60 mL). The resulting mixture was concentrated under reduced pressure. The residue was purified by silica gel column chromatography, eluted with CH₂Cl₂/MeOH (10:1) to afford Compound 197-5 (2.2 g, 80.81%) as a brown solid.

### Synthesis of Compound 197-6.

To a stirred solution of Compound 197-5 (2.2 g, 3.428 mmol, 1 equiv) in DCM (18 mL) was added TFA (4 mL) in portions at room temperature under nitrogen atmosphere. The resulting mixture was stirred for 3 h at room temperature. The resulting mixture was concentrated under reduced pressure. This resulted in Compound 197-6 (1.8 g, 96.94%) as a brown solid.

### Synthesis of Compound 197-7.

A solution of Compound 197-4 (413 mg, 0.923 mmol, 1 equiv) in DMF (6 mL) were treated with HATU (701.86 mg, 1.846 mmol, 2 equiv) and DIEA (477.14 mg, 3.692 mmol, 4 equiv) for 1 h at room temperature under nitrogen atmosphere followed by the addition of Compound 197-6 (599.80 mg, 1.108 mmol, 1.20 equiv) at room temperature. The resulting mixture was stirred for 3 h at room temperature under nitrogen atmosphere. The residue was purified by reversed-phase flash chromatography with the following conditions: column, C18 silica gel; mobile phase, MeCN in water (0.1% FA), 20% to 50% gradient in 30 min; detector, UV 254 nm. This resulted in Compound 197-7 (390 mg, 43.52%) as a yellow solid.

### Synthesis of Compound 197.

The Compound 197-7 (390 mg) was purified by Prep-CHIRAL-HPLC with the following conditions (Column: CHIRALPAK IH, 2*25 cm, 5 μm; Mobile Phase A: Hex (0.2% TEA)—HPLC, Mobile Phase B: IPA:DCM=1:1—HPLC; Flow rate: 20 mL/min; Gradient: 65% B to 65% B in 14 min; Wave Length: 220/254 nm; RT1 (min): 5.35; RT2 (min): 10.03; Sample Solvent: IPA:DCM=1:1, The first peak was the product) to afford Compound 197 (42 mg, 10.50%) as a yellow solid.

LCMS-Compound 197: (ES, m/z): [M+H]⁺ 972

NMR-Compound 197: (400 MHz, DMSO-d6, δ ppm): 1.71-1.80 (m, 9H), 1.84-1.87 (m, 1H), 2.00-2.07 (m, 1H), 2.32-2.33 (m, 1H), 2.50-2.56 (m, 1H), 2.60-2.87 (m, 2H), 2.89-3.11 (m, 5H), 3.09-3.33 (m, 3H), 3.34-3.41 (m, 3H), 3.42-3.72 (m, 3H), 4.01-4.18 (m, 2H), 4.22-4.35 (m, 3H), 5.02-5.07 (m, 1H), 6.69-7.08 (m, 1H), 7.10-7.20 (m, 2H), 7.20-7.21 (m, 1H), 7.29-7.40 (m, 4H), 7.42-7.44 (m, 2H), 7.45-7.47 (m, 1H), 7.52-7.60 (m, 1H), 7.60-7.66 (m, 1H), 7.66-7.75 (m, 1H), 8.20-8.30 (m, 1H), 10.94 (s, 1H).

### Example 196. Compound 198

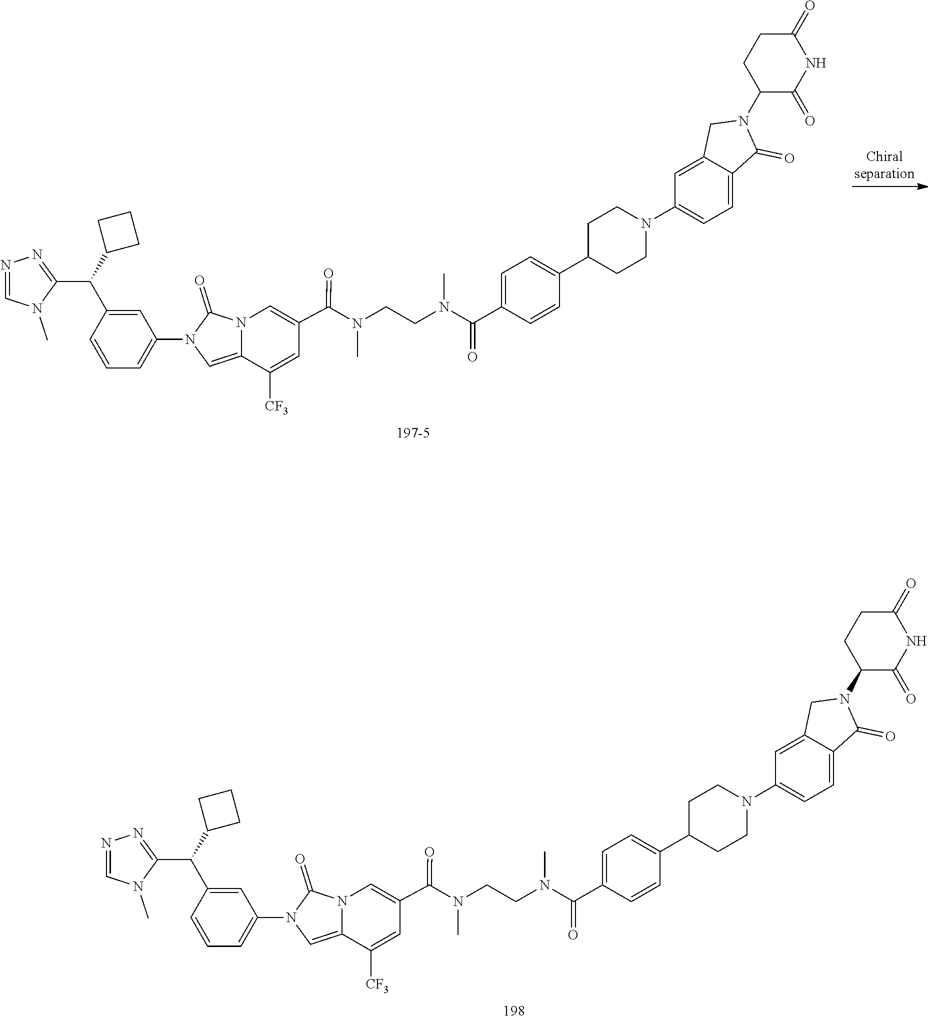 [SMILES: CN(CCN(C)C(=O)c1cc(C(F)(F)F)c2cn(-c3cccc([C@H](c4nncn4C)C4CCC4)c3)c(=O)n2c1)C(=O)c1ccc(C2CCN(c3ccc4c(c3)CN(C3CCC(=O)NC3=O)C4=O)CC2)cc1 ; CN(CCN(C)C(=O)c1cc(C(F)(F)F)c2cn(-c3cccc([C@H](c4nncn4C)C4CCC4)c3)c(=O)n2c1)C(=O)c1ccc(C2CCN(c3ccc4c(c3)CN([C@H]3CCC(=O)NC3=O)C4=O)CC2)cc1]

### Synthesis of Compound 198.

The Compound 197-5 (390 mg) was purified by Prep-Chiral-HPLC with the following conditions (Column: CHIRALPAK IH, 2*25 cm, 5 μm; Mobile Phase A: Hex (0.2% TEA)—HPLC, Mobile Phase B: IPA:DCM=1:1—HPLC; Flow rate: 20 mL/min; Gradient: 65% B to 65% B in 14 min; Wave Length: 220/254 nm; RT1 (min): 5.35; RT2 (min): 10.03; Sample Solvent: IPA:DCM=1:1 The second peak was the product) to afford Compound 198-0 (48 mg, 12.00%) as a yellow solid.

LCMS-Compound 198: (ES, m/z): [M+H]⁺ 972

NMR-Compound 198: (400 MHz, DMSO-d6, δ ppm): 1.71-1.80 (m, 9H), 1.84-1.87 (m, 1H), 2.00-2.07 (m, 1H), 2.32-2.33 (m, 1H), 2.50-2.56 (m, 1H), 2.60-2.87 (m, 2H), 2.89-3.11 (m, 5H), 3.09-3.33 (m, 3H), 3.34-3.41 (m, 3H), 3.42-3.72 (m, 3H), 4.01-4.18 (m, 2H), 4.22-4.35 (m, 3H), 5.02-5.07 (m, 1H), 6.99-7.08 (m, 1H), 7.10-7.20 (m, 2H), 7.20-7.21 (m, 1H), 7.29-7.40 (m, 4H), 7.42-7.44 (m, 2H), 7.45-7.49 (m, 1H), 7.52-7.62 (m, 1H), 7.64-7.67 (m, 1H), 7.68-7.75 (m, 1H), 8.20-8.30 (m, 1H), 10.95 (s, 1H).

### Example 197. Compound 199

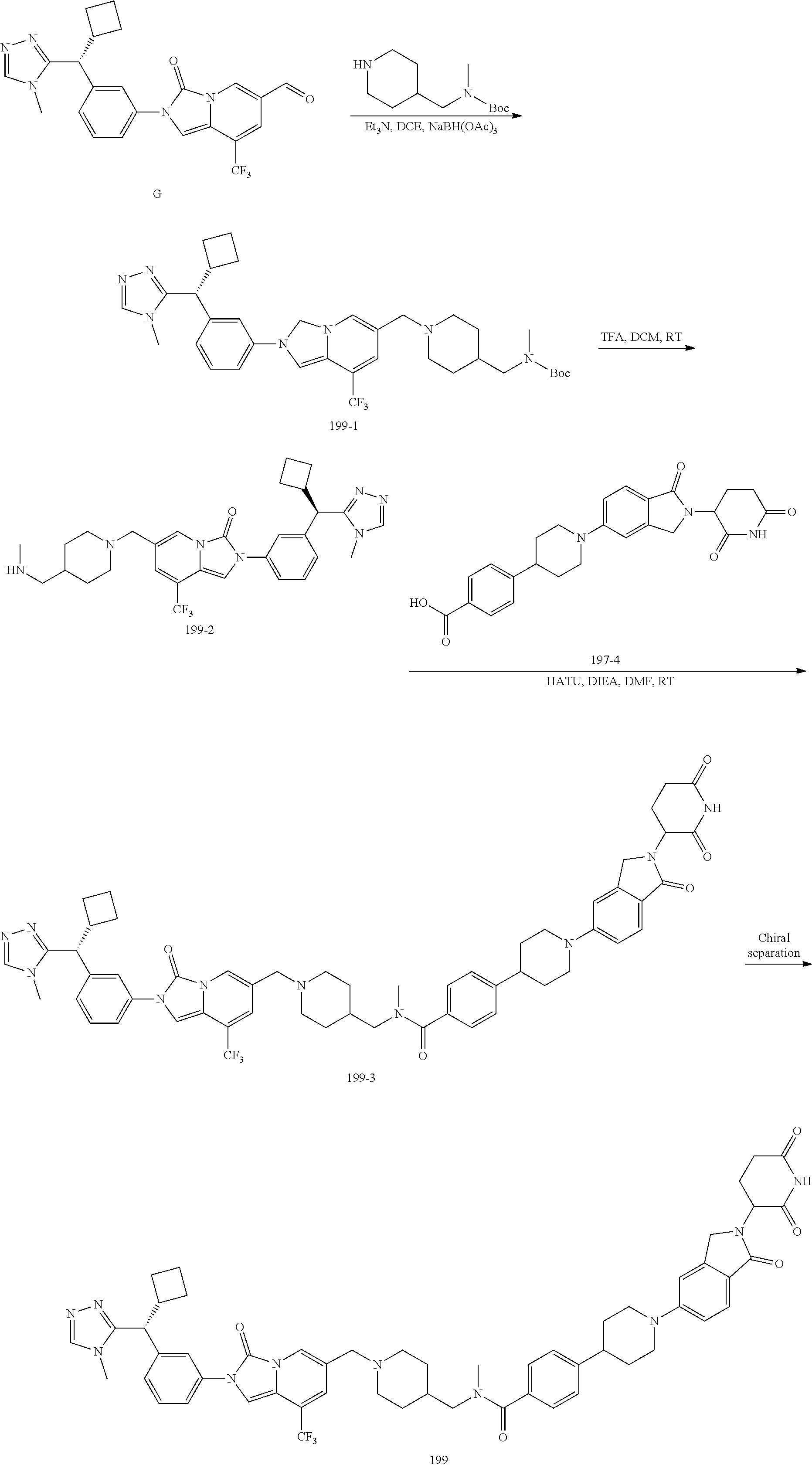

### Synthesis of Compound 199-1.

A solution of tert-butyl N-methyl-N-(piperidin-4-ylmethyl) carbamate (1.50 g, 6.587 mmol, 1.50 equiv) in DCE (20 mL) was treated with Intermediate G (2 g, 4.391 mmol, 1.0) equiv) for overnight at room temperature under nitrogen atmosphere followed by the addition of NaBH(OAc)₃ (1.86 g, 8.782 mmol, 2 equiv) at room temperature. The resulting mixture was stirred for 4 h at room temperature. The reaction was quenched with sat. NH₄Cl (aq.) (50 ml) at room temperature. The resulting mixture was extracted with CH₂Cl₂/MeOH (10:1) (3×100 mL). The combined organic layers were concentrated under reduced pressure. The residue was purified by silica gel column chromatography, eluted with CH₂Cl₂/MeOH (80:1) to afford Compound 199-1 (1.3 g, 44.33%) as a light yellow solid.

### Synthesis of Compound 199-2.

A solution of Compound 199-1 (1.1 g, 1.647 mmol, 1 equiv) in TFA (2 mL) and DCM (10 mL) was stirred for overnight at room temperature. The resulting mixture was concentrated under reduced pressure. The mixture was basified to pH 8 with saturated NaHCO₃ (aq.) (10 mL). The resulting mixture was extracted with CH₂Cl₂/MeOH (10:1) (3×10 mL). The combined organic layers were concentrated under reduced pressure. The residue was purified by Prep-TLC (CH₂Cl₂/MeOH 18:1) to afford Compound 199-2 (500 mg, 53.47%) as a yellow solid.

### Synthesis of Compound 199-3.

A solution of Compound 197-4 (500 mg, 1.117 mmol, 1.00 equiv) in DMF (0.5 mL) were treated with HATU (849.70 mg, 2.234 mmol, 2 equiv) and DIEA (577.65 mg, 4.468 mmol, 4 equiv) for 1 h at room temperature under nitrogen atmosphere followed by the addition of Compound 199-2 (761.13 mg, 1.340 mmol, 1.20 equiv) at room temperature. The resulting mixture was stirred for 4 h at room temperature under nitrogen atmosphere. The resulting mixture was purified by Prep-HPLC with the following conditions (Column: XSelect CSH Fluoro Phenyl, 30*150 mm, 5 μm; Mobile Phase A: Water (0.1% FA), Mobile Phase B: ACN; Flow rate: 60 mL/min; Gradient: 25% B to 37% B in 7 min, 37% B: Wave Length: 254; 220 nm; RT1 (min): 6.98; Number of Runs: 0) to afford Compound 199-3 (420 mg, 37.70%) as a yellow solid.

### Synthesis of Compound 199.

The Compound 199-3 (390 mg) was purified by Prep-CHIRAL-HPLC with the following conditions (Column: CHIRALPAK IH, 2*25 cm, 5 μm; Mobile Phase A: Hex (0.2% TEA)—HPLC, Mobile Phase B: IPA:DCM=1:1—HPLC; Flow rate: 20 mL/min; Gradient: 70% B to 70% B in 26 min; Wave Length: 220/254 nm; RT1 (min): 6.07; RT2 (min): 17.80; Sample Solvent: IPA:DCM=1:1. The first peak was the product) to afford Compound 199 (55.5 mg, 14.23%) as a yellow solid.

LCMS-Compound 199: (ES, m/z): [M+H]⁺ 998

NMR-Compound 199: (400 MHz, DMSO-d6, δ ppm): 0.84.87 (m, 1H), 1.24-1.27 (m, 1H), 1.44-1.47 (m, 1H), 1.73-1.79 (m, 4H), 1.78-1.87 (m, 5H), 1.99-2.04 (m, 5H), 2.04-2.95 (m, 1H), 2.49-2.50 (m, 1H), 2.55-2.61 (m, 2H), 2.56-2.91 (m, 9H), 3.22-3.33 (m, 3H), 3.34-3.42 (m, 3H), 3.42-3.72 (m, 4H), 4.01-4.0 (m, 2H), 4.22-4.35 (m, 3H), 5.02-5.07 (m, 1H), 6.89-7.08 (m, 1H), 7.10-7.20 (m, 2H), 7.20-7.21 (m, 1H), 7.29-7.40 (m, 5H), 7.42-7.47 (m, 2H), 7.47-7.51 (m, 1H), 7.52-7.60 (m, 3H), 8.14 (m, 1H), 8.32 (m, 1H), 10.94 (s, 1H).

### Example 198. Compound 200

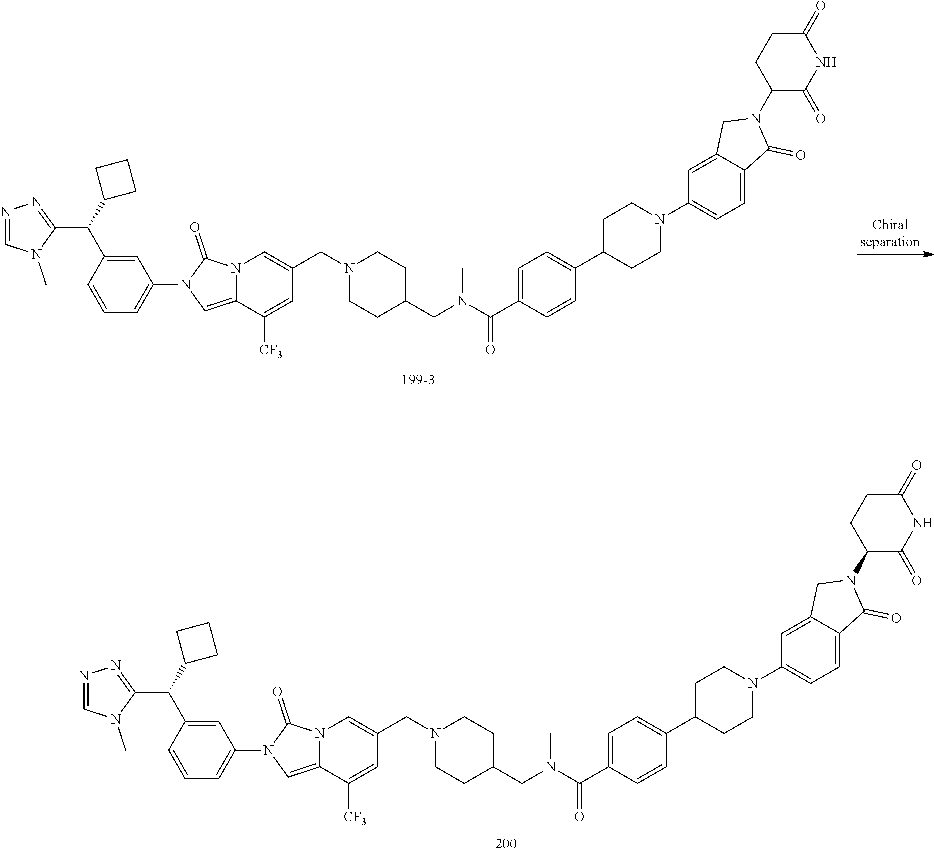 [SMILES: CN(CC1CCN(Cc2cc(C(F)(F)F)c3cn(-c4cccc([C@H](c5nncn5C)C5CCC5)c4)c(=O)n3c2)CC1)C(=O)c1ccc(C2CCN(c3ccc4c(c3)CN(C3CCC(=O)NC3=O)C4=O)CC2)cc1 ; CN(CC1CCN(Cc2cc(C(F)(F)F)c3cn(-c4cccc([C@H](c5nncn5C)C5CCC5)c4)c(=O)n3c2)CC1)C(=O)c1ccc(C2CCN(c3ccc4c(c3)CN([C@H]3CCC(=O)NC3=O)C4=O)CC2)cc1]

### Synthesis of Compound 200.

The Compound 199-3 (390 mg, 0.391 mmol, 1 equiv) was purified by Prep-CHIRAL-HPLC with the following conditions (Column: CHIRALPAK IH, 2*25 cm, 5 μm, Mobile Phase A: Hex (0.2% TEA)—HPLC, Mobile Phase B: IPA:DCM=1:1; Flow rate: 20 mL/min; Gradient: 70% B to 70% B in 26 min; Wave Length: 220/254 nm; RT₁ (min): 6.07; RT₂ (min): 17.80; Sample Solvent: IPA:DCM=1:1, The second peak was the product) to afford Compound 200 (77.6 mg, 19.90%) as a yellow solid.

LCMS-Compound 200: (ES, m/z): [M+H]⁺ 998

NMR-Compound 200: (400 MHz, DMSO-d6, δ ppm): 0.84.87 (m, 1H), 1.24-1.27 (m, 1H), 1.44-1.47 (m, 1H), 1.73-1.79 (m, 4H), 1.78-1.87 (m, 5H), 1.99-2.04 (m, 5H), 2.04-2.95 (m, 1H), 2.49-2.50 (m, 1H), 2.55-2.61 (m, 2H), 2.56-2.91 (m, 9H), 3.22-3.33 (m, 3H), 3.34-3.42 (m, 3H), 3.42-3.72 (m, 4H), 4.01-4.0 (m, 2H), 4.22-4.35 (m, 3H), 5.02-5.07 (m, 1H), 6.89-7.08 (m, 1H), 7.10-7.20 (m, 2H), 7.20-7.21 (m, 1H), 7.29-7.40 (m, 5H), 7.42-7.47 (m, 2H), 7.47-7.51 (m, 1H), 7.52-7.60 (m, 3H), 8.14 (s, 1H), 8.32 (s, 1H), 10.94 (s, 1H).

### Example 199. Compound 201

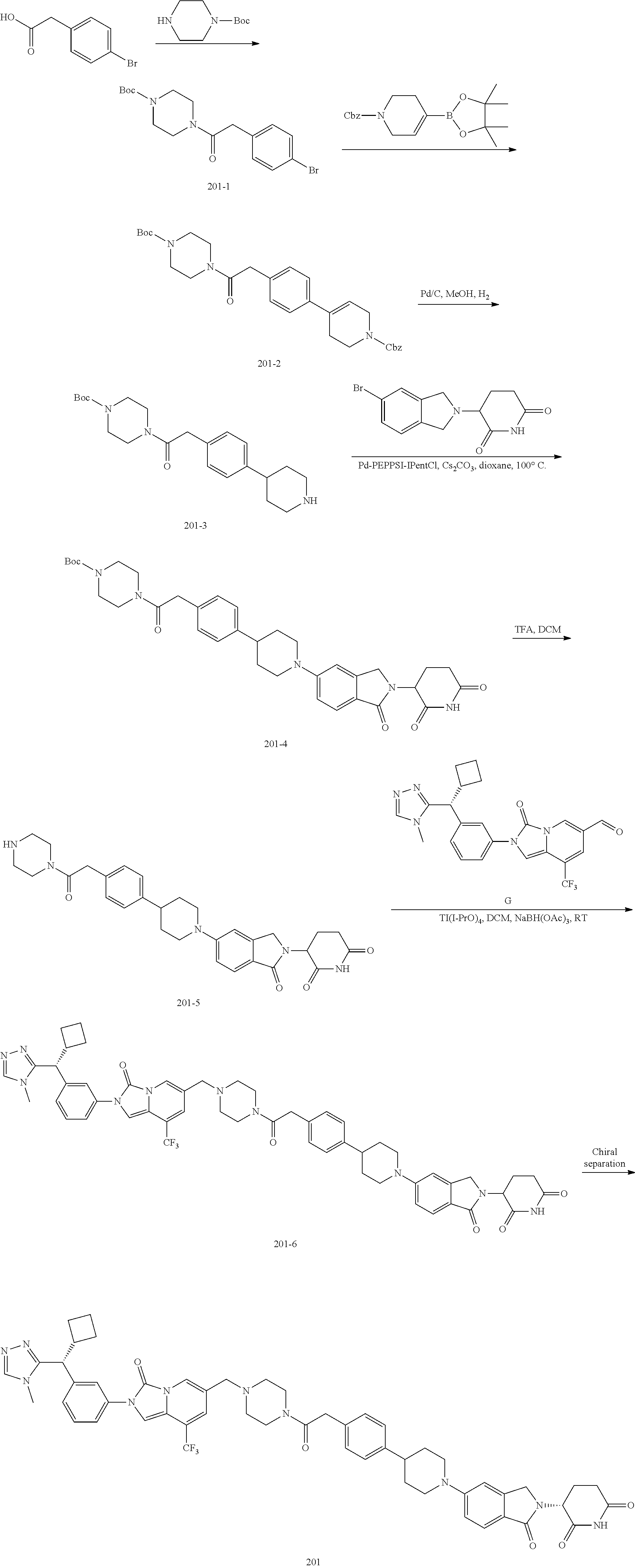

### Synthesis of Compound 201-1.

To a stirred mixture of C₈H₇BrO₂ (19 g, 88.35 mmol, 1.0 equiv) and tert-butyl piperazine-1-carboxylate (20 g, 106.02 mmol, 1.2 equiv) in THF (190 mL) were added T3P (84 g, 265.05 mmol, 3.0 equiv) and DIEA (34 g, 265.05 mmol, 3.0 equiv) at room temperature under nitrogen atmosphere. The resulting mixture was stirred for 2 h at 60° C. under nitrogen atmosphere. The reaction was quenched by the addition of water (500 mL) at room temperature. The aqueous layer was extracted with EtOAc (3×200 mL). The resulting mixture was concentrated under reduced pressure. The residue was purified by silica gel column chromatography, eluted with PE/EA (50:1) to afford Compound 201-1 (30 g, 82%) as an off-white solid.

### Synthesis of Compound 201-2.

To a stirred mixture of Compound 201-1 (30 g, 78.27 mmol, 1.0 equiv) and benzyl 4-(4,4,5,5-tetramethyl-1,3,2-dioxaborolan-2-yl)-3,6-dihydro-2H-pyridine-1-carboxylate (54 g, 156.54 mmol 2.0 equiv) in dioxane (240 mL) H₂O (60 mL) were added Pd(dppf)Cl₂ (11.5 g, 15.65 mmol, 0.2 equiv) and K₃PO₄ (49.8 g, 234.81 mmol, 3.0 equiv) at room temperature under nitrogen atmosphere. The resulting mixture was stirred for overnight at 100° C. under nitrogen atmosphere. The reaction was quenched by the addition of water (500 mL) at room temperature. The aqueous layer was extracted with EtOAc (3×200 mL). The resulting mixture was concentrated under reduced pressure. The residue was purified by silica gel column chromatography, eluted with CH₂Cl₂/MeOH (100:1) to afford Compound 201-2 (32 g, 75%) as a yellow solid.

### Synthesis of Compound 201-3.

To a solution of Compound 201-2 (32 g, 61.58 mmol, 1.0 equiv) in 900 mL MeOH was added Pd/C (10%, 7 g) under nitrogen atmosphere in a 2 L round-bottom flask. The mixture was hydrogenated at room temperature for 2 h under hydrogen atmosphere using a hydrogen balloon, filtered through a Celite pad and concentrated under reduced pressure to afford Compound 201-3 (20 g, 77%) as a yellow solid.

### Synthesis of Compound 201-4.

To a stirred mixture of Compound 201-3 (8 g, 20.64 mmol, 1.0 equiv) in dioxane (80 mL) were added Cs₂CO₃ (20 g, 61.93 mmol, 3.0 equiv) and Pd-PEPPSI-IPentCl (0.35 g, 0.413 mmol, 0.02 equiv) and 3-(5-bromo-1-oxo-3H-isoindol-2-yl) piperidine-2,6-dione (8 g, 24.77 mmol, 1.2 equiv) at room temperature under nitrogen atmosphere. The resulting mixture was stirred for overnight at 100° C. under nitrogen atmosphere. The reaction was quenched by the addition of HOAc (10%) (500 mL) at room temperature. The aqueous layer was extracted with CH₂Cl₂/MeOH (10:1) (3×500 mL). The resulting mixture was concentrated under reduced pressure. The residue was purified by silica gel column chromatography, eluted with CH₂Cl₂/MeOH (50:1) to afford Compound 201-4 (3.5 g, 26%) as a yellow solid.

### Synthesis of Compound 201-5.

A mixture of Compound 201-4 (4 g, 6.35 mmol, 1.0 equiv) in TFA (10 mL) and DCM (30 mL) was stirred for 2 h at room temperature under nitrogen atmosphere. The resulting mixture was concentrated under reduced pressure. This resulted in Compound 201-5 (3 g, 85%) as a yellow solid.

### Synthesis of Compound 201-6.

To a stirred solution of Compound 201-5 (1.2 g, 2.26 mmol, 1.0 equiv) and Intermediate G (1.2 g, 2.71 mmol, 1.2 equiv) in DCM (12 mL) were added Ti(Oi-Pr)₄ (2.5 g, 9.06 mmol, 4.0 equiv) at room temperature. The resulting mixture was stirred for overnight at room temperature. To the above mixture was added NaBH(OAc)₃ (1.4 g, 6.79 mmol, 3.0 equiv) at room temperature. The resulting mixture was stirred for additional 2 h at room temperature. The reaction was quenched by the addition of 10% AcOH (100 mL) at room temperature. The aqueous layer was extracted with DCM:MeOH (10:1) (3×100 mL). The resulting mixture was concentrated under reduced pressure. The residue was purified by reversed-phase flash chromatography with the following conditions: column, C18 silica gel; mobile phase, MeCN in water (0.1% FA), 10% to 50% gradient in 30 min; detector, UV 254 nm. The crude product (700 mg) was purified by Prep-HPLC with the following conditions (Column: Sunfire prep C18 column, 30*150 mm, 5 μm; Mobile Phase A: Water (0.1% FA), Mobile Phase B: ACN; Flow rate: 60 mL/min; Gradient: 27% B to 38% B in 8 min, 38% B; Wave Length: 254; 220 nm; RT1 (min): 7.58; Number of Runs: 0) to afford Compound 201-6 (350 mg, 16%) as a yellow solid.

### Synthesis of Compound 201.

The Compound 201-6 (350 mg) was purified by Prep-CHIRAL-HPLC with the following conditions Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 90% B to 90% B in 13 min; Wave Length: 220/254 nm; RT1 (min): 6.72; RT2 (min): 10.17; Sample Solvent: DCM:ACN=1:1—HPLC; Injection Volume: 0.45 mL; Number Of Runs: 7. The first peak was the product. This resulted in Compound 201 (96 mg, 28%) as a yellow solid.

LC-MS-Compound 201: (ES, m/z): [M+H]⁺ 969

H-NMR-Compound 201: (400 MHz, DMSO-d6, δ ppm): 1.69-1.86 (m, 9H), 1.90-1.95 (m, 1H), 2.03-2.05 (m, 1H), 2.34-2.37 (m, 5H), 2.49-2.51 (m, 1H), 2.60-2.63 (m, 1H), 2.89-2.95 (m, 3H), 3.20-3.25 (m, 1H), 3.29-3.32 (m, 2H), 3.39-3.42 (m, 2H), 3.47-3.50 (m, 3H), 3.66-3.70 (m, 4H), 3.75 (s, 2H), 4.02-4.10 (m, 2H), 4.18-4.35 (m, 3H), 5.02-5.07 (m, 1H), 7.01 (s, 1H), 7.08-7.20 (m, 7H), 7.31 (s, 1H), 7.41-7.45 (m, 1H), 7.50-7.53 (m, 1H), 7.67-7.72 (m, 3H), 8.32 (s, 1H), 10.95 (s, 1H).

### Example 200. Compound 202

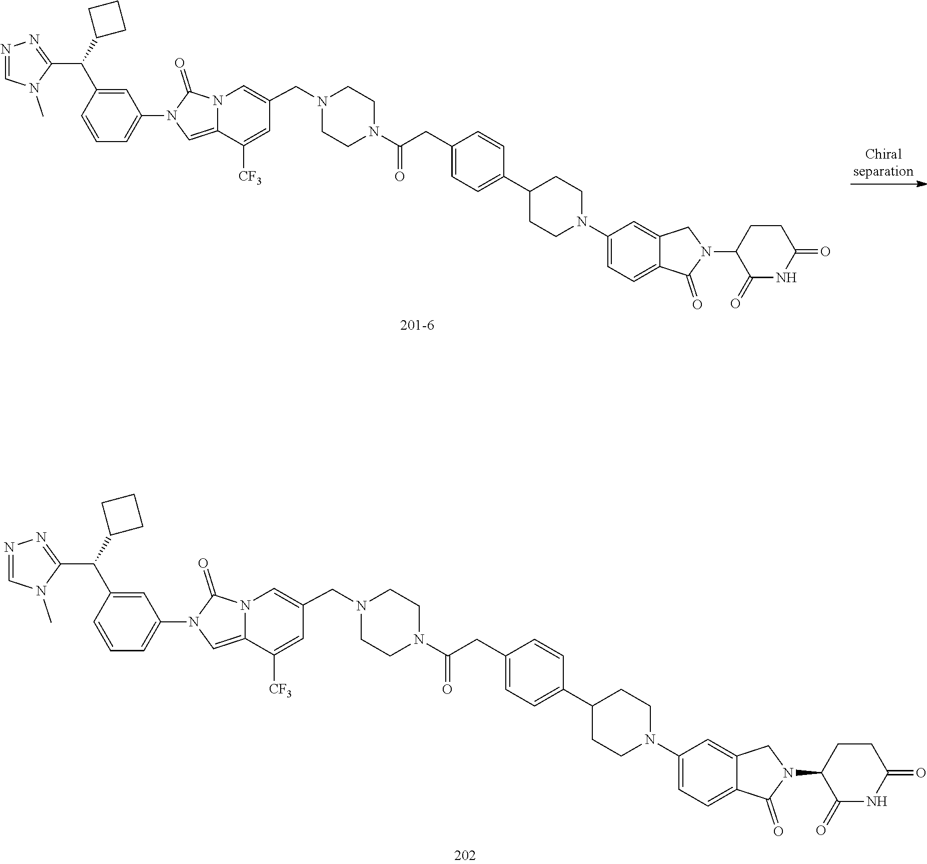 [SMILES: Cn1cnnc1[C@@H](c1cccc(-n2cc3c(C(F)(F)F)cc(CN4CCN(C(=O)Cc5ccc(C6CCN(c7ccc8c(c7)CN(C7CCC(=O)NC7=O)C8=O)CC6)cc5)CC4)cn3c2=O)c1)C1CCC1 ; Cn1cnnc1[C@@H](c1cccc(-n2cc3c(C(F)(F)F)cc(CN4CCN(C(=O)Cc5ccc(C6CCN(c7ccc8c(c7)CN([C@H]7CCC(=O)NC7=O)C8=O)CC6)cc5)CC4)cn3c2=O)c1)C1CCC1]

### Synthesis of Compound 202.

The Compound 201-6 (350 mg) was purified by Prep-CHIRAL-HPLC with the following conditions Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 90% B to 90% B in 13 min; Wave Length: 220/254 nm; RT1 (min): 6.72; RT2 (min): 10.17; Sample Solvent: DCM:ACN=1:1—HPLC; Injection Volume: 0.45 mL; Number Of Runs: 7. The second peak was the product. This resulted in Compound 202 (86 mg, 28%) as a yellow solid.

LC-MS-Compound 202: (ES, m/z): [M+H]⁺ 969

H-NMR-Compound 202: (400 MHz, DMSO-d6, δ ppm): 1.69-1.86 (m, 9H), 1.90-1.95 (m, 1H), 2.03-2.05 (m, 1H), 2.34-2.37 (m, 5H), 2.49-2.51 (m, 1H), 2.60-2.63 (m, 1H), 2.89-2.95 (m, 3H), 3.20-3.25 (m, 1H), 3.29-3.32 (m, 2H), 3.39-3.42 (m, 2H), 3.47-3.50 (m, 3H), 3.66-3.70 (m, 4H), 3.75 (s, 2H), 4.02-4.10 (m, 2H), 4.18-4.35 (m, 3H), 5.02-5.07 (m, 1H), 7.01 (s, 1H), 7.08-7.20 (m, 7H), 7.31 (s, 1H), 7.41-7.45 (m, 1H), 7.50-7.53 (m, 1H), 7.67-7.72 (m, 3H), 8.32 (s, 1H), 10.95 (s, 1H).

### Example 201. Compound 203

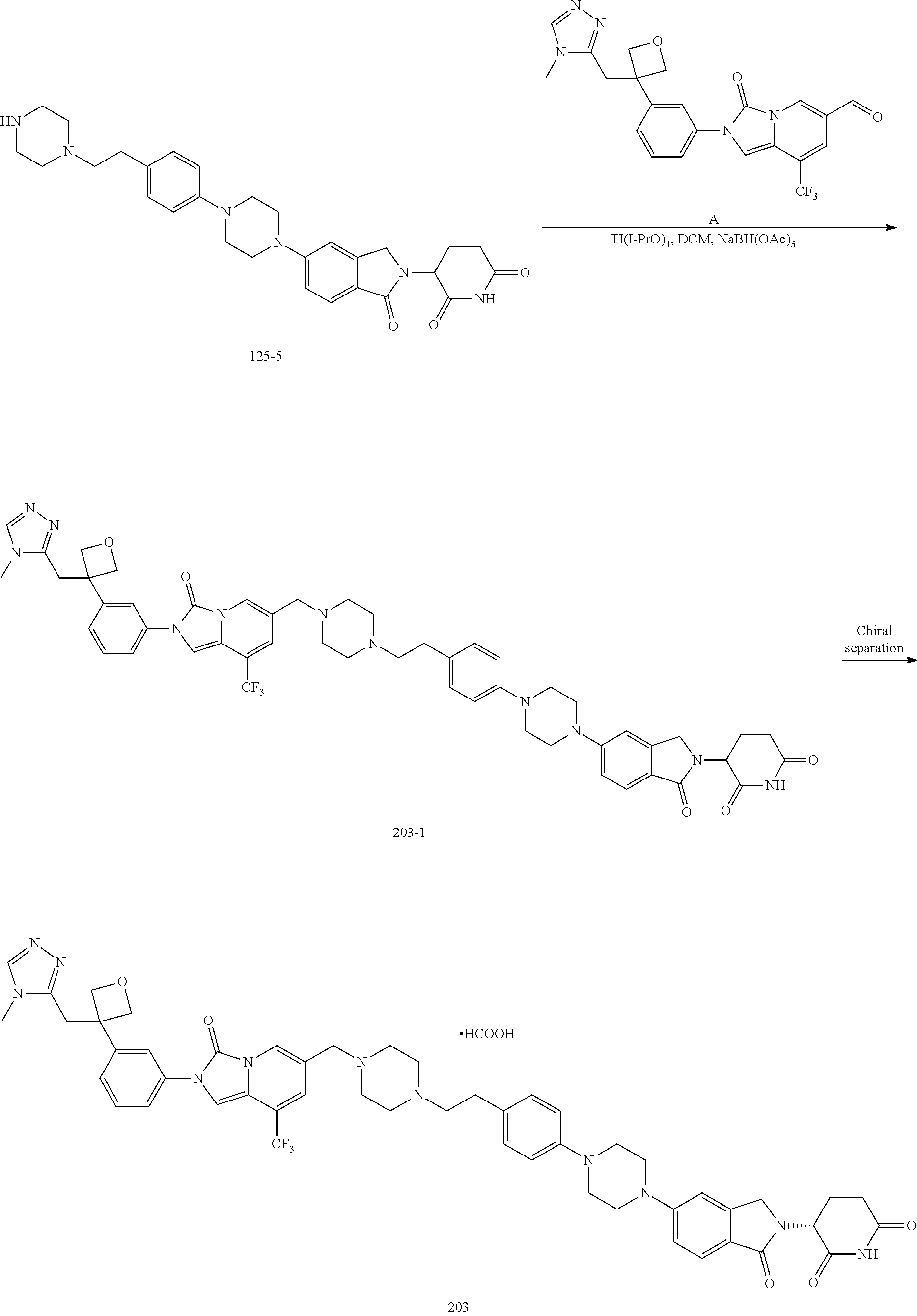

### Synthesis of Compound 203-1.

To a stirred solution of Compound 125-5 (400 mg, 0.774 mmol, 1 equiv) and Intermediate A (389.55 mg, 0.851 mmol, 1.1 equiv) in DCM (4 mL) was added tetrakis(propan-2-yloxy) titanium (660.14 mg, 2.322 mmol, 3 equiv) at room temperature. The resulting mixture was stirred for overnight at room temperature. To the above mixture was added NaBH(OAc)₃ (328.18 mg, 1.548 mmol, 2 equiv) at room temperature. The resulting mixture was stirred for additional 3 h at room temperature. The mixture was acidified to pH 5 with water (10% AcOH) (25 mL). The resulting mixture was filtered; the filter cake was washed with water (10% AcOH) (3×10 mL). The filtrate was concentrated under reduced pressure. The crude product (280 mg) was purified by Prep-HPLC with the following conditions (Column: XSelect CSH Fluoro Phenyl, 30*150 mm, 5 μm; Mobile Phase A: Water (0.1% FA), Mobile Phase B: ACN; Flow rate: 60 mL/min; Gradient: 20% B to 30% B in 8 min, 30% B; Wave Length: 254; 220 nm; RT1 (min): 7.58) to afford Compound 203-1 (120 mg, 15.37%) as a yellow solid.

### Synthesis of Compound 203.

The Compound 203-1 (120 mg, 0.119 mmol, 1 equiv, 95%) was purified by Chiral Separation with the following conditions (Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 80% B to 80% B in 15 min; Wave Length: 220/254 nm; RT1 (min): 8.08; RT2 (min): 12.14; the first peak was product) to afford crude product (55 mg). The crude product (55 mg) was purified by reversed-phase flash chromatography with the following conditions: column, 40 g C18 silica gel; mobile phase, MeCN in Water (0.1% FA), 20% to 45% gradient in 8 min; detector, UV 254 nm to afford Compound 203 (33.3 mg, 28.83%) as a yellow solid.

LC-MS-Compound 203: (ES, m/z): [M+H]⁺ 1004

H-NMR-Compound 203: (400 MHz, DMSO-d6, δ ppm): 1.95-1.99 (m, 1H), 2.36-2.56 (m, 8H), 2.61-2.65 (m, 3H), 2.83-2.96 (m, 5H), 3.24-3.29 (m, 4H), 3.32-3.42 (m, 4H), 3.43-3.45 (m, 4H), 3.53 (s, 2H), 4.20-4.24 (d, 1H), 4.32-4.37 (d, 1H), 4.94-4.95 (m, 4H), 5.03-5.06 (m, 1H), 6.88-6.93 (m, 3H), 7.01 (s, 1H), 7.08-7.14 (m, 4H), 7.31 (s, 1H), 7.37-7.39 (m, 2H), 7.40-7.41 (m, 1H), 7.54-7.56 (m, 1H), 7.67 (s, 1H), 7.73-7.76 (s, 1H), 8.15 (s, 1H), 8.19 (s, 1H), 10.96 (s, 1H).

### Example 202. Compound 204

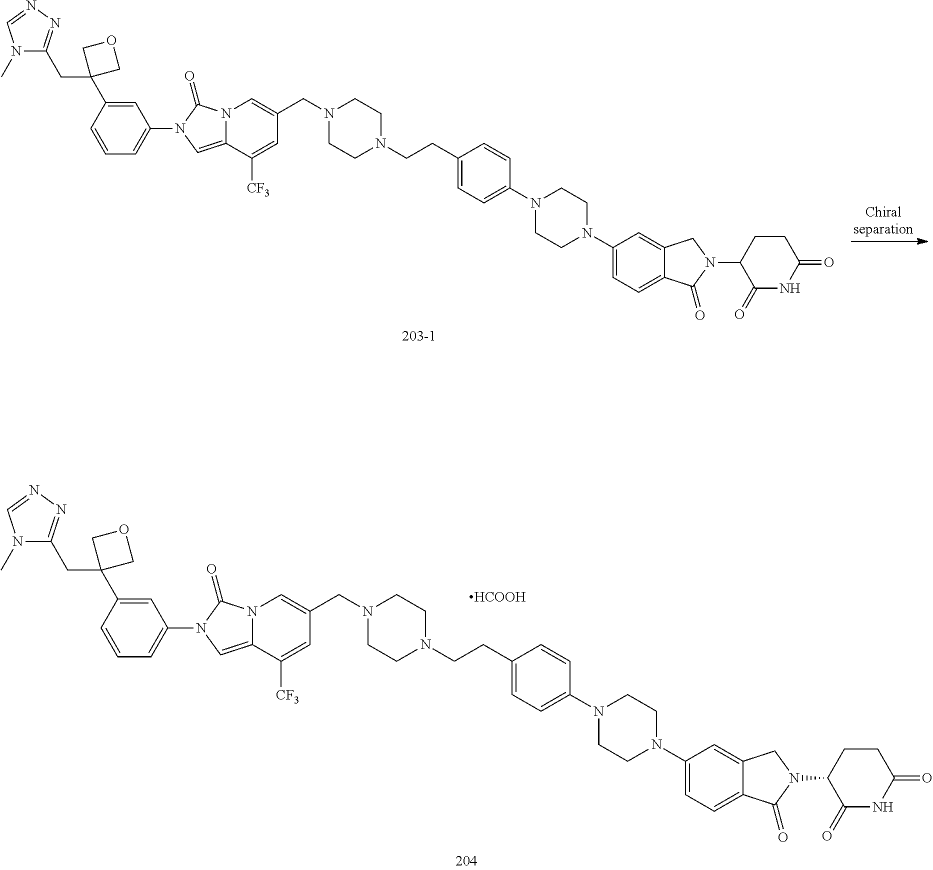 [SMILES: Cn1cnnc1CC1(c2cccc(-n3cc4c(C(F)(F)F)cc(CN5CCN(CCc6ccc(N7CCN(c8ccc9c(c8)CN(C8CCC(=O)NC8=O)C9=O)CC7)cc6)CC5)cn4c3=O)c2)COC1 ; Cn1cnnc1CC1(c2cccc(-n3cc4c(C(F)(F)F)cc(CN5CCN(CCc6ccc(N7CCN(c8ccc9c(c8)CN([C@@H]8CCC(=O)NC8=O)C9=O)CC7)cc6)CC5)cn4c3=O)c2)COC1 ; "·HCOOH"]

### Synthesis of Compound 204.

The Compound 203-1 (120 mg, 0.119 mmol, 1 equiv, 95%) was purified by Chiral Separation with the following conditions (Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 80% B to 80% B in 15 min; Wave Length: 220/254 nm; RT1 (min): 8.08; RT2 (min): 12.14; the first second was product) to afford crude product (50 mg). The crude product (50 mg) was purified by reversed-phase flash chromatography with the following conditions: column, 40 g C18 silica gel; mobile phase, MeCN in Water (0.1% FA), 20% to 45% gradient in 8 min; detector, UV 254 nm to afford Compound 204 (29.7 mg, 25.27%) as a yellow solid.

LC-MS-Compound 204: (ES, m/z): [M+H]⁺ 1003

H-NMR-Compound 204: (400 MHz, DMSO-d6, δ ppm): 1.95-1.99 (m, 1H), 2.32-2.56 (m, 7H), 2.61-2.67 (m, 4H), 2.83-2.96 (m, 5H), 3.24-3.37 (m, 4H), 3.32-3.42 (m, 4H), 3.43-3.45 (m, 4H), 3.53 (s, 2H), 4.20-4.37 (m, 2H), 4.94-4.95 (m, 4H), 5.04-5.06 (m, 1H), 6.88-6.93 (m, 3H), 7.01 (s, 1H), 7.08-7.14 (m, 4H), 7.31 (s, 1H), 7.37-7.39 (m, 2H), 7.40-7.41 (m, 1H), 7.54-7.56 (m, 1H), 7.67 (s, 1H), 7.73-7.76 (s, 1H), 8.15 (s, 1H), 8.19 (s, 1H), 10.96 (s, 1H).

### Example 203. Compound 205

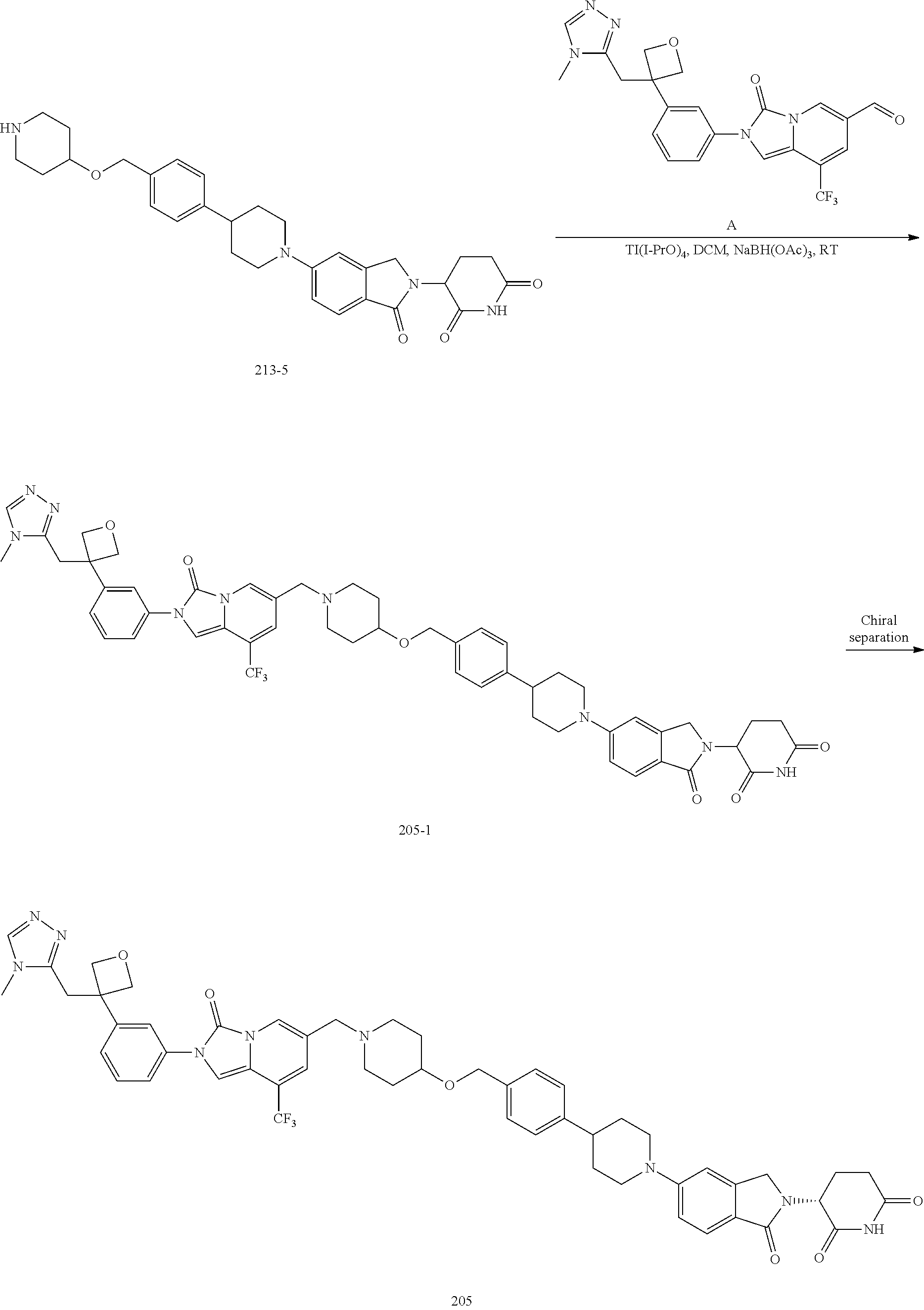

### Synthesis of Compound 205-1.

To a stirred solution of Compound 213-5 (1.2 g, 2.323 mmol, 1 equiv) and Intermediate A (1.06 g, 2.323 mmol, 1 equiv) in DCM (30 mL) was added Ti(Oi-Pr)₄ (1.98 g, 6.969 mmol, 3 equiv) at room temperature. The resulting mixture was stirred overnight at room temperature. To the above mixture was added STAB (1.97 g, 9.292 mmol, 4 equiv) at room temperature. The resulting mixture was stirred for additional 4 h at room temperature. The reaction was quenched with 10% HOAc (aq.) at room temperature. The aqueous layer was extracted with DCM (3×100 mL). The resulting mixture was concentrated under vacuum. The residue was purified by reversed-phase flash chromatography with the following conditions (column, C18 silica gel; mobile phase, MeCN in Water (0.1% FA), 10% to 50% gradient in 30 min; detector, UV 254 nm) to afford crude product. The crude product was purified by Prep-HPLC with the following conditions (Column: Sunfire prep C18 column, 30*150 mm, 5 μm; Mobile Phase A: Water (0.1% FA), Mobile Phase B: ACN; Flow rate: 60 mL/min; Gradient: 23% B to 35% B in 10 min; Wave Length: 254; 220 nm; RT1 (min): 9.02) to afford Compound 205-1 (200 mg, 8.63%) as a yellow solid.

### Synthesis of Compound 205.

The Compound 205-1 (200 mg) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 90% B to 90% B in 11 min; Wave Length: 220/254 nm; RT1 (min): 6.33; RT2 (min): 8.74; the first peak is product) to afford the crude product. The crude product was purified by reverse flash chromatography with the following conditions (column, C18 silica gel; mobile phase, MeCN in water (0.1% FA), 0% to 30% gradient in 10 min; detector, UV 254 nm): 6.03) to afford Compound 205 (66.7 mg, 32.42%) as a yellow solid.

LC-MS-Compound 205: (ES, m/z): [M+H]⁺ 958

H-NMR-Compound 205: (400 MHz, DMSO-d₆, δ ppm): 1.41-1.61 (m, 2H), 1.68-2.01 (m, 7H), 2.11-2.23 (m, 2H), 2.31-2.45 (m, 1H), 2.53-2.83 (m, 4H), 2.87-2.96 (m, 6H), 3.28 (s, 2H), 3.33-3.40 (m, 1H), 3.53 (s, 2H), 4.00-4.03 (m, 1H), 4.18-4.35 (m, 2H), 4.45 (s, 2H), 4.90-4.95 (m, 4H), 5.02-5.07 (d, 1H), 6.87-6.89 (d, 1H), 7.01 (s, 1H), 7.09-7.11 (m, 2H), 7.21-7.32 (m, 5H), 7.37-7.41 (m, 2H), 7.51-7.66 (m, 2H), 7.74-7.76 (d, 1H), 8.20 (s, 1H), 10.95 (s, 1H).

### Example 204. Compound 206

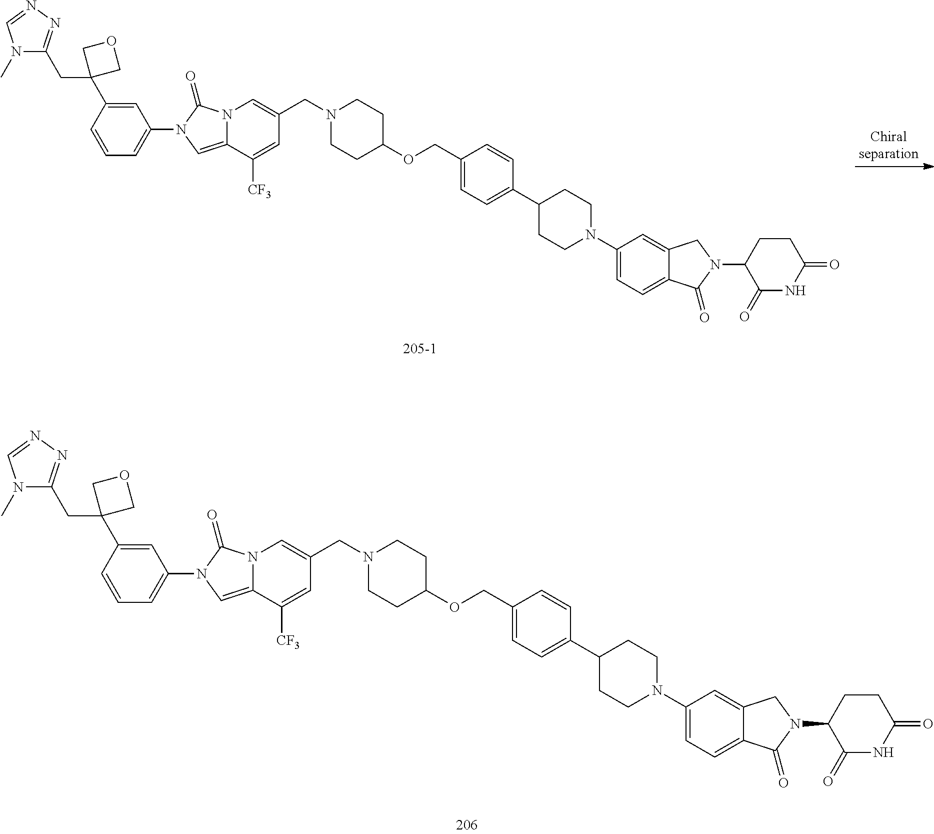 [SMILES: Cn1cnnc1CC1(c2cccc(-n3cc4c(C(F)(F)F)cc(CN5CCC(OCc6ccc(C7CCN(c8ccc9c(c8)CN(C8CCC(=O)NC8=O)C9=O)CC7)cc6)CC5)cn4c3=O)c2)COC1 ; Cn1cnnc1CC1(c2cccc(-n3cc4c(C(F)(F)F)cc(CN5CCC(OCc6ccc(C7CCN(c8ccc9c(c8)CN([C@H]8CCC(=O)NC8=O)C9=O)CC7)cc6)CC5)cn4c3=O)c2)COC1]

### Synthesis of Compound 206.

The Compound 205-1 (200 mg) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 90% B to 90% B in 11 min; Wave Length: 220/254 nm; RT1 (min): 6.33; RT2 (min): 8.74; the second peak is product) to afford the crude product. The crude product was purified by reverse flash chromatography with the following conditions (column, C18 silica gel; mobile phase, MeCN in water (0.1% FA), 0% to 30% gradient in 10 min; detector, UV 254 nm): 6.03) to afford Compound 206 (58.0 mg, 28.91%) as a yellow solid.

LC-MS-Compound 206: (ES, m/z): [M+H]⁺ 958

H-NMR-Compound 206: (400 MHz, DMSO-d₆, δ ppm): 1.41-1.61 (m, 2H), 1.68-2.01 (m, 7H), 2.11-2.23 (m, 2H), 2.31-2.45 (m, 1H), 2.53-2.83 (m, 4H), 2.87-2.96 (m, 6H), 3.28 (s, 2H), 3.33-3.40 (m, 1H), 3.53 (s, 2H), 4.00-4.03 (m, 1H), 4.18-4.23 (m, 1H), 4.28-4.31 (m, 1H), 4.45 (s, 2H), 4.90-4.95 (m, 4H), 5.02-5.07 (d, 1H), 6.87-6.89 (d, 1H), 7.01 (s, 1H), 7.09-7.11 (m, 2H), 7.21-7.26 (m, 4H), 7.32 (s, 1H), 7.37-7.41 (m, 2H), 7.51-7.53 (d, 1H), 7.66 (s, 1H), 7.74-7.76 (d, 1H), 8.20 (s, 1H), 10.95 (s, 1H).

### Example 205. Compound 207

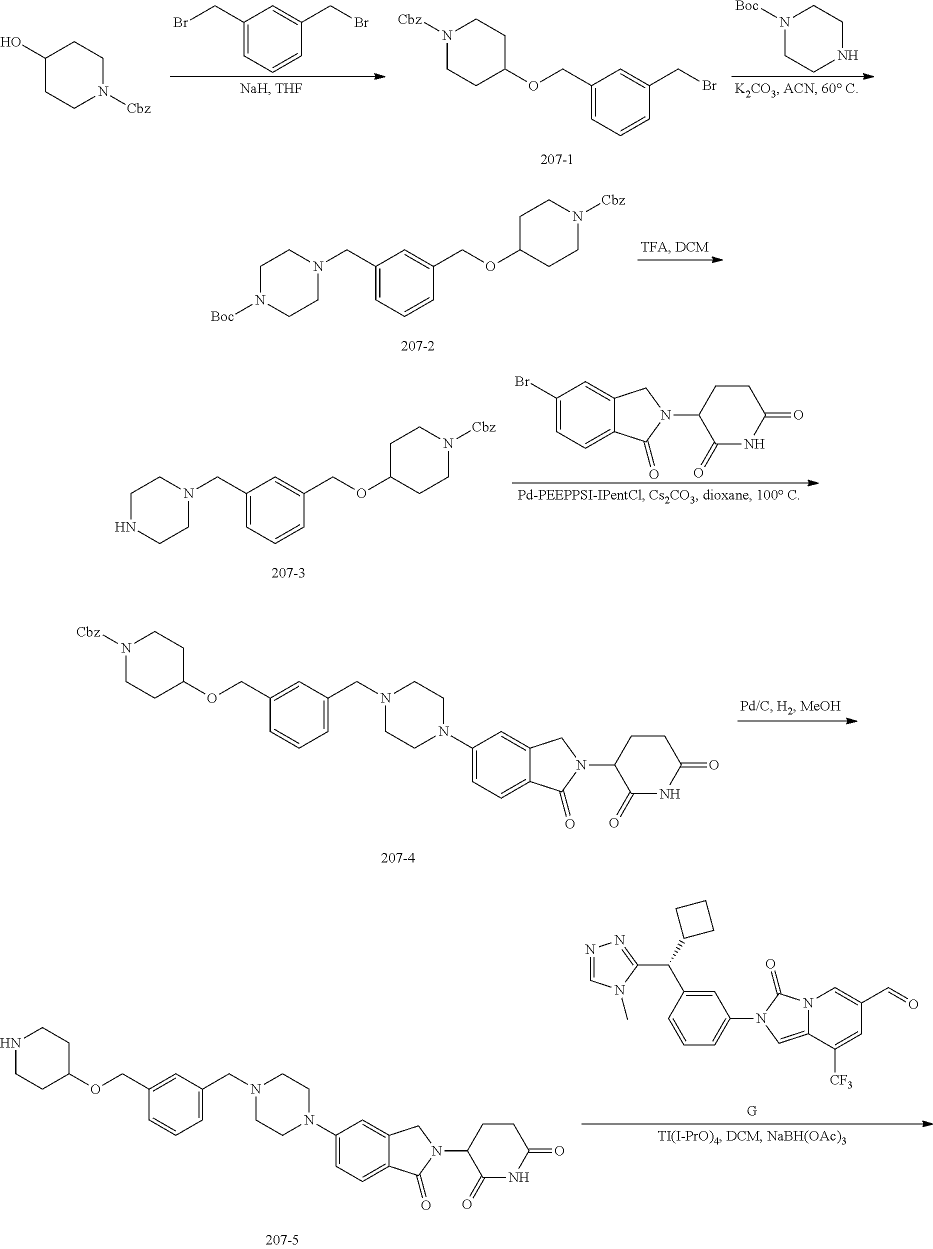

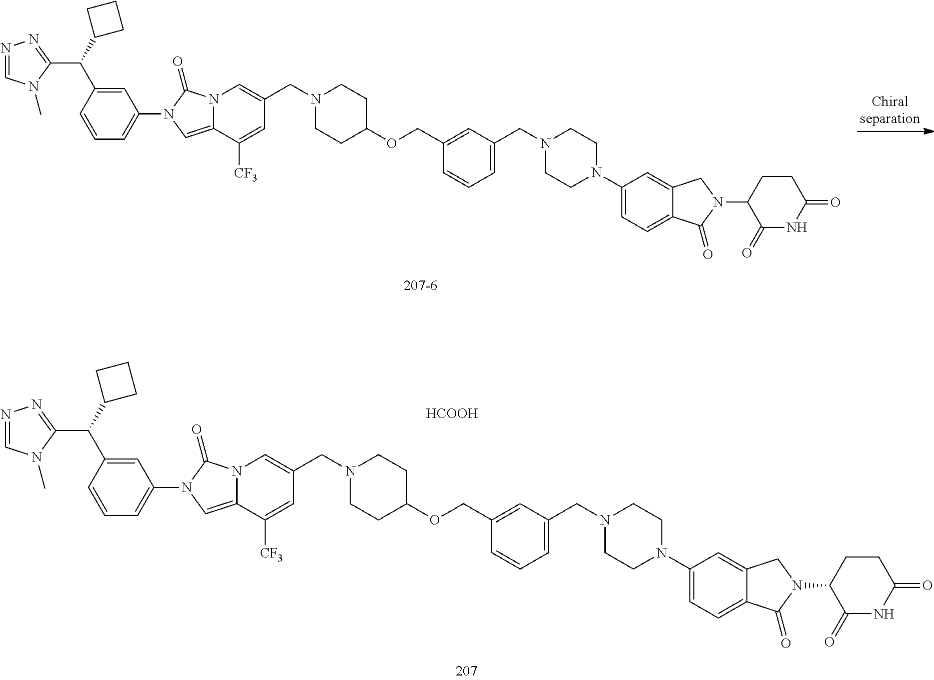 [SMILES: Cn1cnnc1[C@@H](c1cccc(-n2cc3c(C(F)(F)F)cc(CN4CCC(OCc5cccc(CN6CCN(c7ccc8c(c7)CN(C7CCC(=O)NC7=O)C8=O)CC6)c5)CC4)cn3c2=O)c1)C1CCC1 ; Cn1cnnc1[C@@H](c1cccc(-n2cc3c(C(F)(F)F)cc(CN4CCC(OCc5cccc(CN6CCN(c7ccc8c(c7)CN([C@@H]7CCC(=O)NC7=O)C8=O)CC6)c5)CC4)cn3c2=O)c1)C1CCC1 ; "HCOOH"]

### Synthesis of Compound 207-1.

To a solution of benzyl 4-hydroxypiperidine-1-carboxylate (20 g, 85.00 mmol, 1.0 equiv) in THF (200 mL) was added sodium hydride (60% in oil, 2.5 g) at 0 degrees C. The mixture was stirred for 1 h. benzene, 1,3-bis(bromomethyl)-(26.9 g, 102.00 mmol, 1.2 equiv) was added and the mixture was allowed to warm to room temperature and stirred for 3 h. The reaction mixture was quenched by water (400 mL) and extracted with DCM (3×200 mL). The residue was purified by silica gel column chromatography, eluted with CH₂Cl₂/MeOH (20:1) to afford Compound 207-1 (20 g, 51%) as a white solid.

### Synthesis of Compound 207-2.

To a stirred solution of Compound 207-1 (20 g, 47.80 mmol, 1.0 equiv) and Compound 207-2 (10.7 g, 57.37 mmol, 1.2 equiv) in MeCN (250 ml) was added K₂CO₃ (13 g, 95.61 mmol, 2.0 equiv) at room temperature. The resulting mixture was stirred for overnight at 60° C. The reaction was quenched by the addition of sat. NH₄Cl (aq.) (900 mL) at room temperature. The aqueous layer was extracted with EtOAc (3×300 mL). The resulting mixture was concentrated under vacuum. The residue was purified by silica gel column chromatography, eluted with PE/EA (1:1) to afford Compound 207-2 (14 g, 54%) as a light yellow oil.

### Synthesis of Compound 207-3.

Into a 500 mL 3-necked round-bottom flask were added TFA (40 mL) and DCM (120 mL) at room temperature. To the above mixture was added Compound 207-2 (13 g, 24.82 mmol, 1.0 equiv) at room temperature. The resulting mixture was stirred for additional overnight at room temperature. The resulting mixture was concentrated under reduced pressure. The resulting mixture was filtered; the filter cake was washed with EtOAc (3×60 mL). The filtrate was concentrated under reduced pressure. The residue was purified by trituration with MeCN (200 mL). This resulted in Compound 207-3 (11 g, 96%) as a pink solid.

### Synthesis of Compound 207-4.

To a stirred solution of Compound 207-3 (14 g, 33.05 mmol, 1.0 equiv) and 3-(5-bromo-1-oxo-3H-isoindol-2-yl) piperidine-2,6-dione (13.9 g, 42.96 mmol, 1.3 equiv) in dioxane (120 mL) was added Cs₂CO₃ (21.5 g, 66.10 mmol, 2.0 equiv) and Pd-PEPPSI-IPentCl 2-methylpyridine (o-picoline (1.4 g, 1.65 mmol, 0.05 equiv) under nitrogen atmosphere. The resulting mixture was stirred overnight at 100° C. under nitrogen atmosphere. The mixture was allowed to cool down to room temperature. The mixture was acidified to pH 6 with 10% HOAc. The resulting mixture was concentrated under vacuum. The residue was purified by silica gel column chromatography, eluted with CH₂Cl₂ (0.1% HOAc)/MeOH (20:1) to afford Compound 207-4 (1.4 g, 6%) as a brown solid.

### Synthesis of Compound 207-5.

Into a 50 mL round-bottom flask were added Compound 207-4 (1 g, 1.50 mmol, 1.0 equiv), Pd/C (100 mg) and MeOH (30 mL) at room temperature under hydrogen atmosphere. The resulting mixture was stirred for additional 2 h at room temperature. The resulting mixture was filtered; the filter cake was washed with DCM (3×30 mL). The filtrate was concentrated under reduced pressure. The residue was purified by reversed-phase flash chromatography with the following conditions: column, C18 silica gel; mobile phase, MeCN in water (0.1% FA), 10% to 50% gradient in 30 min; detector, UV 254 nm. This resulted in Compound 207-5 (500 mg, 63%) as an off-white solid.

### Synthesis of Compound 207-6.

To a stirred solution of Compound 207-5 (680 mg, 1.27 mmol, 1.0 equiv) and Intermediate G (699 mg, 1.53 mmol, 1.2 equiv) in DCM (6 mL) were added Ti(Oi-Pr)₄ (1.4 g, 5.11 mmol, 4.0 equiv) and NaBH(OAc)₃ (813 mg, 3.83 mmol, 3.0 equiv) at room temperature. The resulting mixture was stirred for overnight at room temperature. To the above mixture was added NaBH(OAc) (813 mg, 3.83 mmol, 3.0 equiv) at room temperature. The resulting mixture was stirred for additional 2 h at room temperature. The reaction was quenched by the addition of 10% AcOH (100 mL) at room temperature. The aqueous layer was extracted with CH₂Cl₂:MeOH (5:1) (3×100 mL). The residue was purified by reversed-phase flash chromatography with the following conditions: column, C18 silica gel; mobile phase, MeCN in water (0.1% FA), 10% to 50% gradient in 30 min; detector, UV 254 nm. The crude product (400 mg) was purified by Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 10% B to 22% B in 11 min, 22% B; Wave Length: 254; 220 nm; RT1 (min): 10.5; Number of Runs: 0) to afford Compound 207-6 (110 mg, 9%) as a yellow solid.

### Synthesis of Compound 207.

The Compound 207-6 (110 mg) was purified by Prep-CHIRAL-HPLC with the following conditions Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 90% B to 90% B in 8 min; Wave Length: 220/254 nm; RT1 (min): 4.36; RT2 (min): 6.44; Sample Solvent: DCM:ACN=1:1—HPLC; Injection Volume: 1 mL; Number Of Runs: 3. The first peak was the product. The residue was purified by reversed-phase flash chromatography with the following conditions: column, C18 silica gel; mobile phase, MeCN in water (0.1% FA), 10% to 50% gradient in 30 min; detector, UV 254 nm. This resulted in Compound 207 (30 mg, 26%) as a yellow solid.

LC-MS-Compound 207: (ES, m/z): [M-COOH]⁺ 971

H-NMR-Compound 207: (400 MHz, DMSO-d6, δ ppm): 1.51-1.53 (m, 2H), 1.74-1.85 (m, 8H), 2.07-2.17 (m, 3H), 2.30-2.33 (m, 1H), 2.48-2.50 (m, 1H), 2.59-2.69 (m, 2H), 2.89-2.95 (m, 1H), 3.22-3.27 (m, 2H), 3.33-3.35 (m, 3H), 3.37-3.39 (m, 4H), 3.42-3.44 (m, 5H), 3.52-3.55 (m, 3H), 4.17-4.29 (m, 3H), 4.50 (s, 2H), 5.01-5.06 (m, 1H), 7.01-7.05 (m, 3H), 7.18-7.24 (m, 3H), 7.29-7.32 (m, 3H), 7.41-7.45 (m, 1H), 7.50-7.52 (d, 1H), 7.65-7.73 (m, 3H), 8.17 (s, 1H), 8.32 (s, 1H), 10.95 (s, 1H).

### Example 206. Compound 208

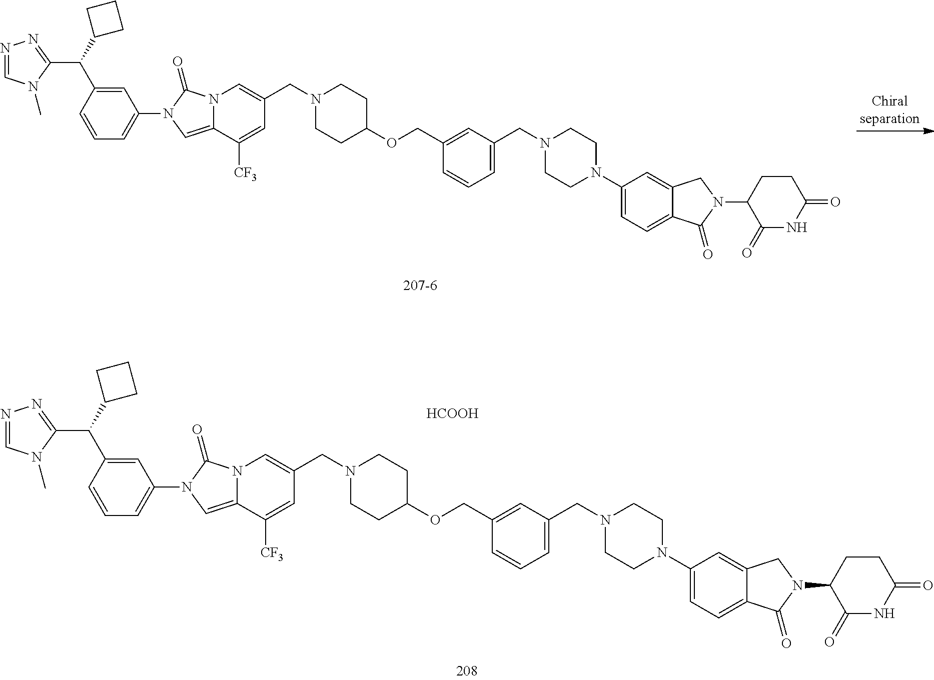 [SMILES: Cn1cnnc1[C@@H](c1cccc(-n2cc3c(C(F)(F)F)cc(CN4CCC(OCc5cccc(CN6CCN(c7ccc8c(c7)CN(C7CCC(=O)NC7=O)C8=O)CC6)c5)CC4)cn3c2=O)c1)C1CCC1 ; Cn1cnnc1[C@@H](c1cccc(-n2cc3c(C(F)(F)F)cc(CN4CCC(OCc5cccc(CN6CCN(c7ccc8c(c7)CN([C@H]7CCC(=O)NC7=O)C8=O)CC6)c5)CC4)cn3c2=O)c1)C1CCC1 ; "HCOOH"]

### Synthesis of Compound 208.

The Compound 207-6 (110 mg) was purified by Prep-CHIRAL-HPLC with the following conditions Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 90% B to 90% B in 8 min; Wave Length: 220/254 nm; RT1 (min): 4.36; RT2 (min): 6.44; Sample Solvent: DCM:ACN=1:1—HPLC; Injection Volume: 1 mL; Number Of Runs: 3. The second peak was the product. The residue was purified by reversed-phase flash chromatography with the following conditions: column, C18 silica gel; mobile phase, MeCN in water (0.1% FA), 10% to 50% gradient in 30 min; detector, UV 254 nm. This resulted in Compound 208 (33 mg, 28%) as a yellow solid.

LC-MS-Compound 208: (ES, m/z): [M-COOH]⁺ 971

H-NMR-Compound 208: (400 MHz, DMSO-d6, δ ppm): 1.51-1.53 (m, 2H), 1.74-1.85 (m, 8H), 2.07-2.17 (m, 3H), 2.30-2.33 (m, 1H), 2.48-2.50 (m, 1H), 2.59-2.69 (m, 2H), 2.89-2.95 (m, 1H), 3.22-3.27 (m, 2H), 3.33-3.35 (m, 3H), 3.37-3.39 (m, 4H), 3.42-3.44 (m, 5H), 3.52-3.55 (m, 3H), 4.17-4.29 (m, 3H), 4.50 (s, 2H), 5.01-5.06 (m, 1H), 7.01-7.05 (m, 3H), 7.18-7.24 (m, 3H), 7.29-7.32 (m, 3H), 7.41-7.45 (m, 1H), 7.50-7.52 (d, 1H), 7.65-7.73 (m, 3H), 8.17 (s, 1H), 8.32 (s, 1H), 10.95 (s, 1H).

### Example 207. Compound 209

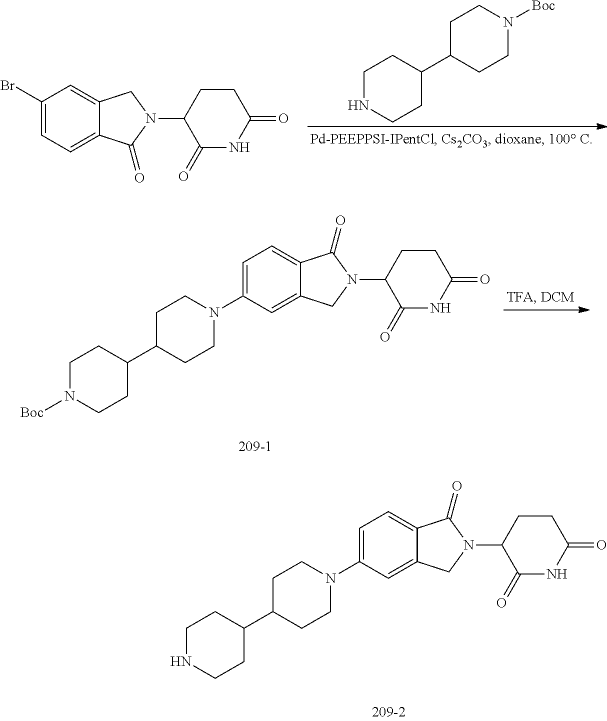 [SMILES: O=C1CCC(N2Cc3cc(Br)ccc3C2=O)C(=O)N1 ; CC(C)(C)OC(=O)N1CCC(C2CCNCC2)CC1 ; CC(C)(C)OC(=O)N1CCC(C2CCN(c3ccc4c(c3)CN(C3CCC(=O)NC3=O)C4=O)CC2)CC1 ; O=C1CCC(N2Cc3cc(N4CCC(C5CCNCC5)CC4)ccc3C2=O)C(=O)N1]

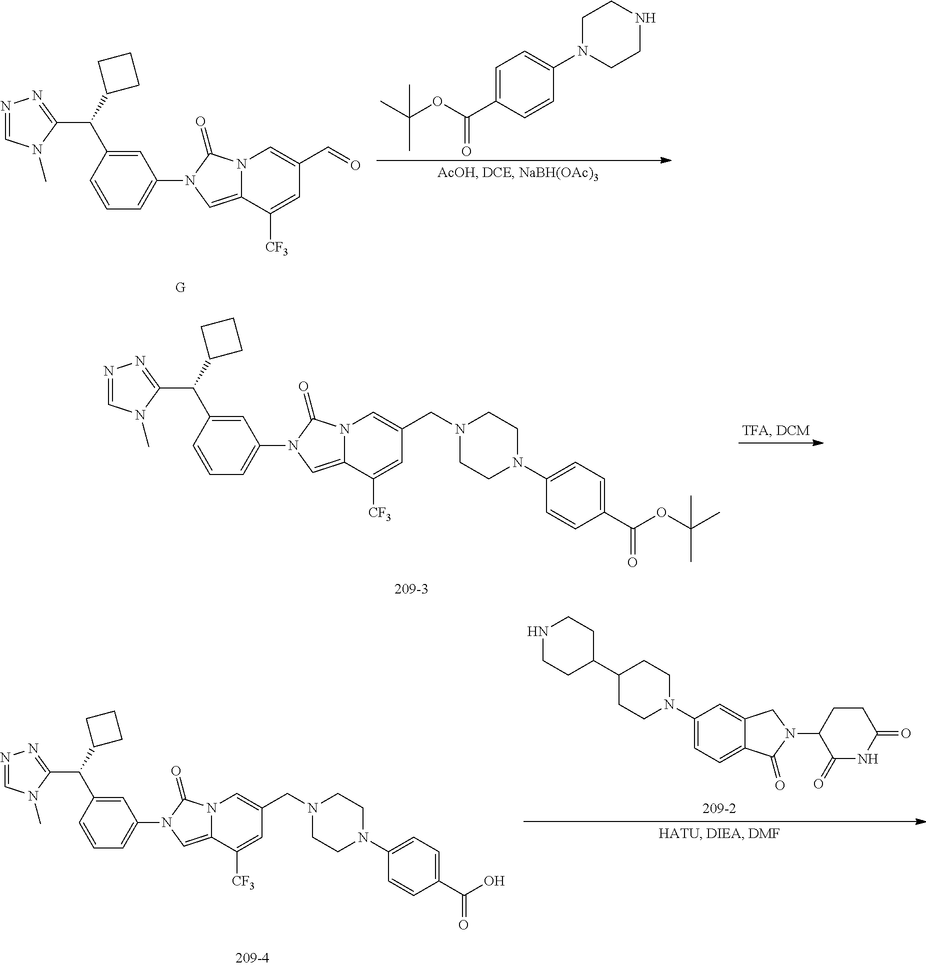 [SMILES: Cn1cnnc1[C@@H](c1cccc(-n2cc3c(C(F)(F)F)cc(C=O)cn3c2=O)c1)C1CCC1 ; CC(C)(C)OC(=O)c1ccc(N2CCNCC2)cc1 ; Cn1cnnc1[C@@H](c1cccc(-n2cc3c(C(F)(F)F)cc(CN4CCN(c5ccc(C(=O)OC(C)(C)C)cc5)CC4)cn3c2=O)c1)C1CCC1 ; Cn1cnnc1[C@@H](c1cccc(-n2cc3c(C(F)(F)F)cc(CN4CCN(c5ccc(C(=O)O)cc5)CC4)cn3c2=O)c1)C1CCC1 ; O=C1CCC(N2Cc3cc(N4CCC(C5CCNCC5)CC4)ccc3C2=O)C(=O)N1]

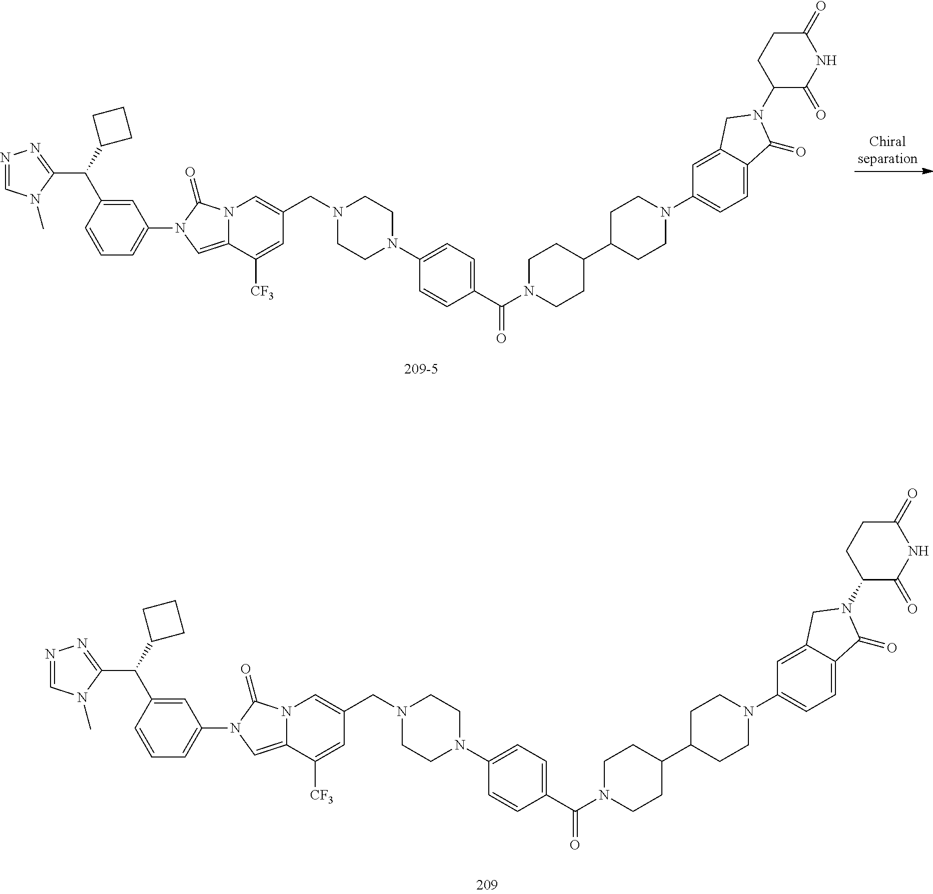 [SMILES: Cn1cnnc1[C@@H](c1cccc(-n2cc3c(C(F)(F)F)cc(CN4CCN(c5ccc(C(=O)N6CCC(C7CCN(c8ccc9c(c8)CN(C8CCC(=O)NC8=O)C9=O)CC7)CC6)cc5)CC4)cn3c2=O)c1)C1CCC1 ; Cn1cnnc1[C@@H](c1cccc(-n2cc3c(C(F)(F)F)cc(CN4CCN(c5ccc(C(=O)N6CCC(C7CCN(c8ccc9c(c8)CN([C@@H]8CCC(=O)NC8=O)C9=O)CC7)CC6)cc5)CC4)cn3c2=O)c1)C1CCC1]

### Synthesis of Compound 209-1.

To a stirred solution of 3-(5-bromo-1-oxo-3H-isoindol-2-yl)piperidine-2,6-dione (5.0 g, 15.47 mmol, 1.0 equiv) and tert-butyl [4,4′-bipiperidine]-1-carboxylate (4.1 g, 15.47 mmol, 1.0 equiv) in 1,4-dioxane (50 mL) were added Cs₂CO₃ (15.1 g, 46.42 mmol, 3.0 equiv) and Pd-PEPPSI-IPentCl 2-methylpyridine (o-picoline) (5.2 mg, 0.006 mmol, 0.1 equiv) at room temperature under nitrogen atmosphere. The resulting mixture was stirred for overnight at 100° C. under nitrogen atmosphere. The reaction was quenched by the addition of 10% AcOH (500 mL) at room temperature. The aqueous layer was extracted with CH₂Cl₂/MeOH=10:1 (3×300 mL). The resulting mixture was concentrated under reduced pressure. The residue was purified by silica gel column chromatography, eluted with CH₂Cl₂/MeOH=20:1 to afford Compound 209-1 (4.0 g, 50%) as a off-white solid.

### Synthesis of Compound 209-2.

A solution of Compound 209-1 (4.0 g, 7.83 mmol, 1.0 equiv) and TFA (10 mL) in DCM (30 mL) was stirred for overnight at room temperature. The resulting mixture was concentrated under reduced pressure. The residue was purified by reversed-phase flash chromatography with the following conditions: column, C18 silica gel; mobile phase, MeCN in water (0.1% FA), 10% to 50% gradient in 30 min; detector, UV 254 nm. This resulted in Compound 209-2 (3.0 g, 93%) as a off-white solid.

### Synthesis of Compound 209-3.

To a stirred solution of Intermediate G (3.0 g, 6.59 mmol, 1.0 equiv) and tert-butyl 4-(piperazin-1-yl)benzoate (1.7 g, 6.59 mmol, 1.0 equiv) in DCE (30 mL) were added HOAc (400 mg, 6.59 mmol, 1.0 equiv) and NaBH(OAc)₃ (2.8 g, 13.17 mmol, 2.0 equiv) at room temperature. The resulting mixture was stirred for overnight at room temperature. The reaction was quenched by the addition of sat. NH₄Cl (aq.) (100 mL) at room temperature. The resulting mixture was extracted with CH₂Cl₂ (3×100 mL). The combined organic layers were washed with water (3×100 mL), dried over anhydrous Na₂SO₄. After filtration, the filtrate was concentrated under reduced pressure. The residue was purified by silica gel column chromatography, eluted with CH₂Cl₂/MeOH=30:1 to afford Compound 209-3 (2.0 g, 43%) as a yellow solid.

### Synthesis of Compound 209-4.

To a stirred solution of Compound 209-3 (2.0 g, 2.82 mmol, 1.0 equiv) in DCM (15 mL) was added TFA (5 mL) at room temperature under nitrogen atmosphere. The resulting mixture was stirred for overnight at room temperature under nitrogen atmosphere. The resulting mixture was concentrated under reduced pressure. The residue was purified by reversed-phase flash chromatography with the following conditions: column, C18 silica gel; mobile phase, MeCN in water (0.1% FA), 10% to 70% gradient in 30 min; detector, UV 254 nm. This resulted in Compound 209-4 (1.1 g, 57.36%) as a yellow solid.

### Synthesis of Compound 209-5.

To a stirred solution of Compound 209-4 (500 mg, 0.77 mmol, 1.0 equiv) in DMF (5 mL) were added DIEA (300 mg, 2.32 mmol, 3.0 equiv), HATU (589 mg, 1.548 mmol, 2.0 equiv) and Compound 209-2 (381 mg, 0.93 mmol, 1.2 equiv) at room temperature under nitrogen atmosphere. The resulting mixture was stirred for overnight at room temperature under nitrogen atmosphere. The reaction was quenched by the addition of water/AcOH=10:1=(60 mL) at room temperature. The aqueous layer was extracted with CH₂Cl₂/MeOH=5:1 (3×50 mL). The resulting mixture was concentrated under reduced pressure. The residue was purified by reversed-phase flash chromatography with the following conditions: column, C18 silica gel; mobile phase, MeCN in water (0.1% FA), 10% to 60% gradient in 30 min; detector, UV 254 nm. This resulted in Compound 209-5 (490 mg, 54%) as a yellow solid.

### Synthesis of Compound 209.

The Compound 209-5 (490 mg, 0.47 mmol, 1.0 equiv) was purified by Prep-Chiral-HPLC with the following conditions (Column: CHIRALPAK IH, 2*25 cm, 5 μm, Mobile Phase A: Hex (0.2% TEA), Mobile Phase B: IPA:DCM=1:1; Flow rate: 20 mL/min; Gradient: 70% B to 70% B in 27 min; Wave Length: 220/254 nm; RT1 (min): 11.02; RT2 (min): 19.02; Sample Solvent: DCM:ACN=1:1; the first peak was product) to afford crude product. The crude product (160 mg) was purified by Prep-Achiral-SFC with the following conditions (Column: GreenSep Basic, 3*15 cm, 5 μm; Mobile Phase A: CO₂, Mobile Phase B: MeOH (0.1% FA); Flow rate: 75 mL/min; Gradient: isocratic 42% B; Column Temperature (° C.): 35; Back Pressure (bar): 100; Wave Length: 254 nm; RT1 (min): 4.62; Sample Solvent: MeOH (0.1% FA); to afford Compound 209 (56 mg, 11%) as a yellow solid.

LCMS-Compound 209: (ES, m/z): [M+H]⁺ 1038

NMR-Compound 209: (400 MHz, DMSO-d6, δ ppm): 1.11-1.33 (m, 7H), 1.70-1.81 (m, 9H), 1.91-1.99 (m, 1H), 2.11-2.19 (m, 1H), 2.32-2.49 (m, 1H), 2.50-2.60 (m, 5H), 2.72-2.89 (m, 5H), 3.23-3.32 (m, 5H), 3.36-3.42 (m, 2H), 3.43-3.45 (m, 3H), 3.89-3.93 (m, 2H), 4.17-4.33 (m, 4H), 5.02-5.06 (m, 1H), 6.93-6.95 (d, 2H), 7.03-7.05 (t, 3H), 7.19-7.26 (m, 3H), 7.32 (s, 1H), 7.42-7.50 (m, 2H), 7.69-7.74 (m, 3H), 8.33 (s, 1H), 10.96 (s, 1H).

### Example 208. Compound 210

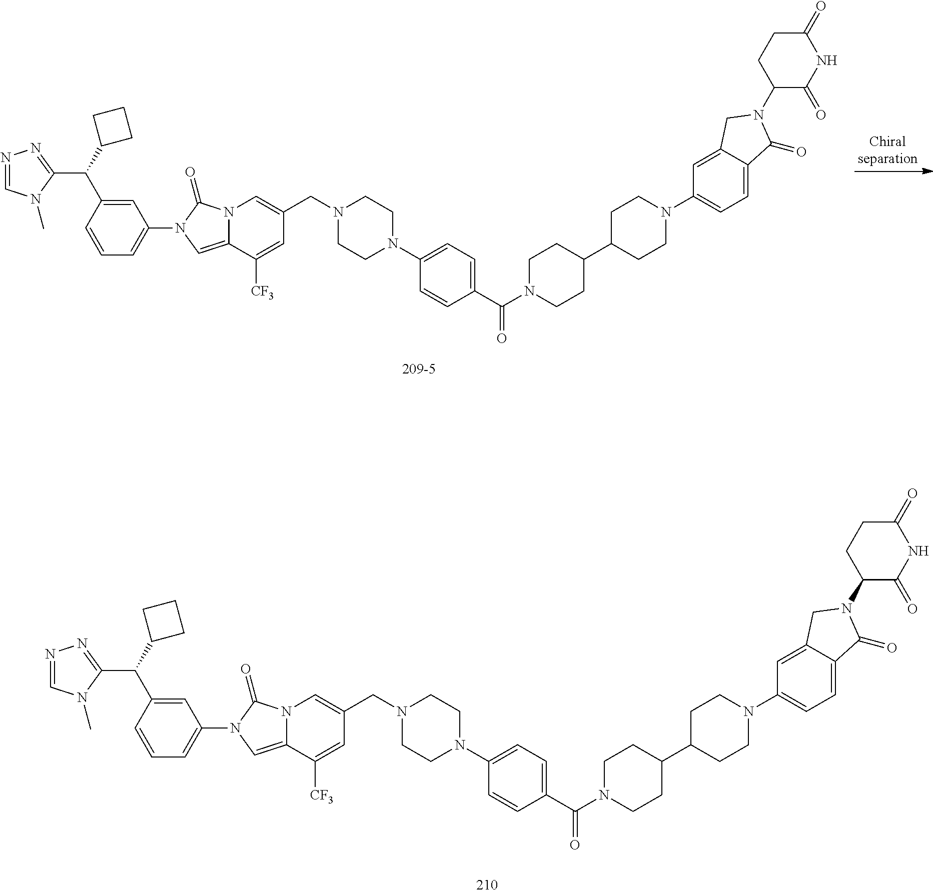 [SMILES: Cn1cnnc1[C@@H](c1cccc(-n2cc3c(C(F)(F)F)cc(CN4CCN(c5ccc(C(=O)N6CCC(C7CCN(c8ccc9c(c8)CN(C8CCC(=O)NC8=O)C9=O)CC7)CC6)cc5)CC4)cn3c2=O)c1)C1CCC1 ; Cn1cnnc1[C@@H](c1cccc(-n2cc3c(C(F)(F)F)cc(CN4CCN(c5ccc(C(=O)N6CCC(C7CCN(c8ccc9c(c8)CN([C@H]8CCC(=O)NC8=O)C9=O)CC7)CC6)cc5)CC4)cn3c2=O)c1)C1CCC1]

### Synthesis of Compound 210.

The Compound 209-5 (490 mg, 0.47 mmol, 1.0 equiv) was purified by Prep-Chiral-HPLC with the following conditions (Column: CHIRALPAK IH, 2*25 cm, 5 μm; Mobile Phase A: Hex (0.2% TEA), Mobile Phase B: IPA:DCM=1:1; Flow rate: 20 mL/min; Gradient: 70% B to 70% B in 27 min; Wave Length: 220/254 nm; RT1 (min): 11.02; RT2 (min): 19.02; the second peak was product) to afford Compound 210 (148 mg, 28%) as a yellow solid.

LCMS-Compound 210: (ES, m/z): [M+H]⁺ 1038

NMR-Compound 210: (400 MHz, DMSO-d6, δ ppm): 1.03-1.29 (m, 7H), 1.70-1.81 (m, 9H), 1.91-1.99 (m, 1H), 2.01-2.09 (m, 1H), 2.32-2.49 (m, 1H), 2.49-2.51 (m, 4H), 2.51-2.78 (m, 5H), 3.23-3.31 (m, 6H), 3.32-3.36 (m, 4H), 3.89-4.33 (m, 7H), 5.03-5.05 (m, 1H), 6.93-6.95 (d, 2H), 7.03-7.05 (t, 3H), 7.19-7.21 (d, 1H), 7.24-7.26 (d, 2H), 7.33 (s, 1H), 7.42-7.44 (t, 1H), 7.48-7.51 (d, 1H), 7.71-7.74 (m, 3H), 8.33 (s, 1H), 10.97 (s, 1H).

### Example 209. Compound 211

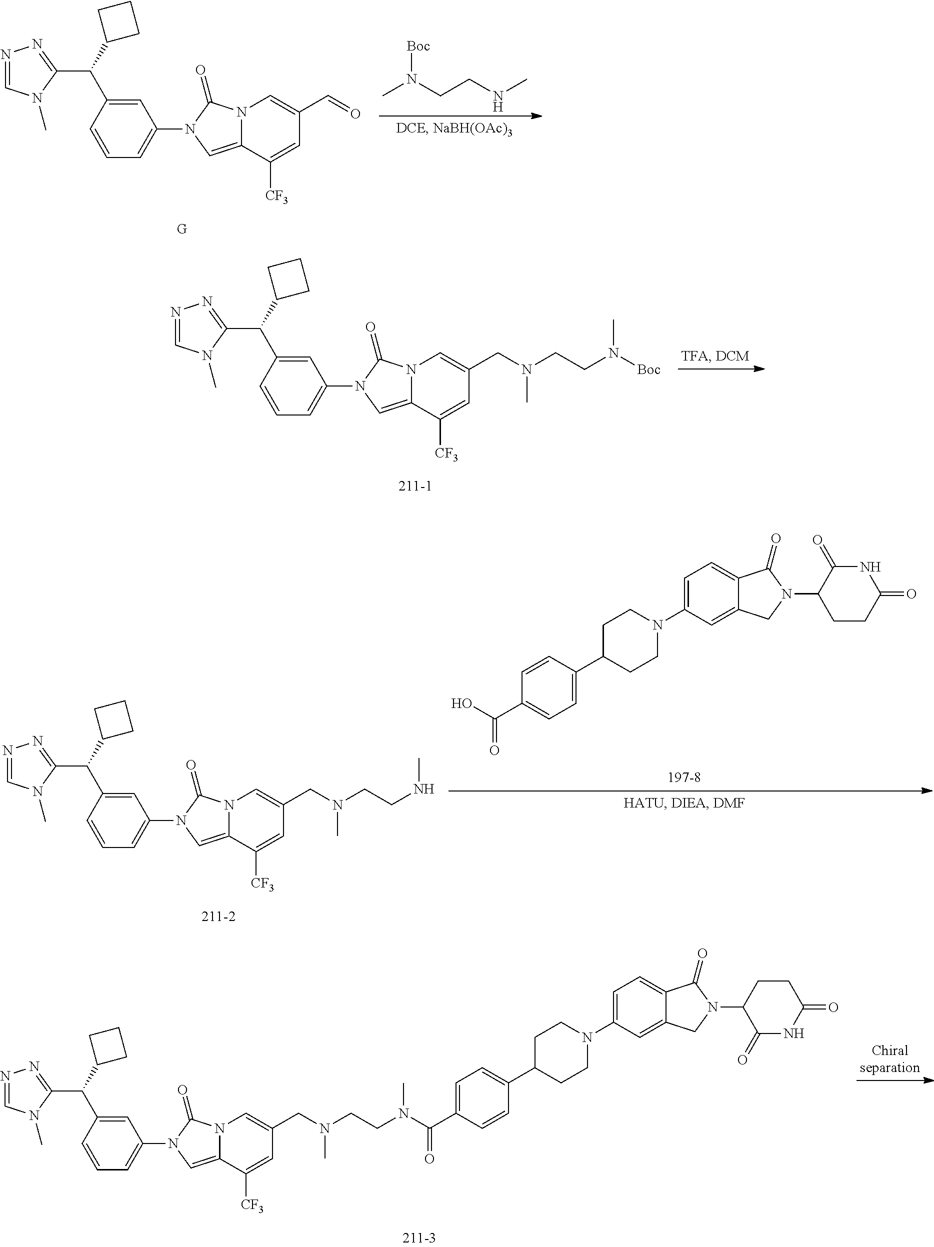

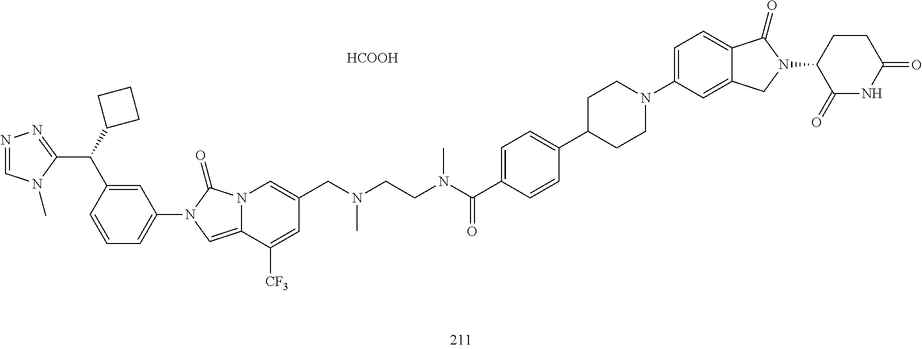 [SMILES: CN(CCN(C)C(=O)c1ccc(C2CCN(c3ccc4c(c3)CN([C@@H]3CCC(=O)NC3=O)C4=O)CC2)cc1)Cc1cc(C(F)(F)F)c2cn(-c3cccc([C@H](c4nncn4C)C4CCC4)c3)c(=O)n2c1 ; "HCOOH"]

### Synthesis of Compound 211-1.

A solution of Intermediate G (2 g, 4.391 mmol, 1 equiv) in DCE (20 mL) was treated with tert-butyl N-methyl-N-[2-(methylamino) ethyl] carbamate (0.99 g, 5.269 mmol, 1.2 equiv) for 0.5 h at room temperature under nitrogen atmosphere followed by the addition of NaBH(OAc)₃ (1.40 g, 6.587 mmol, 1.5 equiv) at room temperature. The resulting mixture was stirred for overnight at room temperature under nitrogen atmosphere. The mixture was acidified to pH 7 with AcOH (10%) (aq.). The aqueous layer was extracted with CH₂Cl₂/MeOH (10:1) (3×50 mL). The resulting mixture was concentrated under vacuum. The residue was purified by silica gel column chromatography, eluted with CH₂Cl₂/MeOH (50:1) to afford Compound 211-1 (1.48 g, 52.08%) as a yellow solid.

### Synthesis of Compound 211-2.

A solution of Compound 211-1 (1.48 g, 2.358 mmol, 1 equiv) in DCM (12 mL) was treated with TFA (4 mL) for 1 h at room temperature under nitrogen atmosphere. The mixture was basified to pH 7 with saturated Na₂CO₃ (aq.). The aqueous layer was extracted with CH₂Cl₂/MeOH (10:1) (3×100 ml). The resulting mixture was concentrated under vacuum. This resulted in Compound 211-2 (927 mg, 71.54%) as a yellow oil.

### Synthesis of Compound 211-3.

A solution of Compound 197-8 (769.29 mg, 1.719 mmol, 1 equiv) in DMF (9 mL) was treated with HATU (980.50 mg, 2.579 mmol, 1.5 equiv) and DIEA (444.38 mg, 3.438 mmol, 2 equiv) for 1 h at 0° C. under nitrogen atmosphere followed by the addition of Compound 211-2 (907 mg, 1.719 mmol, 1 equiv) at room temperature. The resulting mixture was stirred for overnight at room temperature under nitrogen atmosphere. The reaction was quenched with water (10 mL) at room temperature. The crude product (527 mg) was purified by Prep-HPLC with the following conditions (Column: CHIRALPAK IG, 2*25 cm, 5 μm; Mobile Phase A: Hex (0.5% 2M NH3-MeOH)—HPLC, Mobile Phase B: EtOH:DCM=1:1—HPLC; Flow rate: 20 mL/min; Gradient: 40% B to 40% B in 25 min; Wave Length: 220/254 nm; RT1 (min): 11.71; RT2 (min): 17.07; Sample Solvent: EtOH:DCM=1:1—HPLC; Injection Volume: 0.7 mL; Number Of Runs: 4) to afford Compound 211-3 (247 mg, 30.43%) as a white solid.

### Synthesis of Compound 211.

The Compound 211-3 (247 mg) was purified by Prep-CHIRAL-HPLC with the following conditions (Column: CHIRALPAK IH, 2*25 cm, 5 μm; Mobile Phase A: Hex (0.2% TEA)—HPLC, Mobile Phase B: IPA:DCM=1:1—HPLC; Flow rate: 20 mL/min; Gradient: 70% B to 70% B in 16 min; Wave Length: 220/254 nm; RT1 (min): 11.51; RT2 (min): 12.75; Sample Solvent: IPA:DCM=1:1—HPLC; Injection Volume: 0.85 mL; Number of Runs: 10; Number of Runs: 0). The first peak was the product. The residue was purified by reversed-phase flash chromatography with the following conditions: column, C18 silica gel; mobile phase, MeCN in water (0.1% FA), 10% to 50% gradient in 30 min; detector, UV 254 nm. This resulted in Compound 211 (14.7 mg, 2.65%) as a yellow solid.

LCMS-Compound 211: (ES, m/z): [M-COOH]⁺ 957

NMR-Compound 211: (400 MHz, DMSO-d6, ppm): δ 1.41-1.80 (m, 10H), δ1.90-2.01 (m, 3H), δ2.07-2.08 (d, 1H), δ2.25 (s, 2H), δ2.32-2.39 (m, 2H), δ2.50-2.61 (m, 2H), δ2.66-2.67 (m, 1H), δ2.85-2.95 (m, 6H), δ3.12-3.20 (m, 3H), δ3.42 (s, 4H), δ3.59 (s, 1H), δ3.99 (s, 2H), δ4.19-4.35 (m, 3H), δ5.03-5.07 (m, 1H), δ7.03-7.11 (m, 3H), δ7.17-7.19 (d, 1H), δ7.27-7.32 (m, 5H), δ7.41-7.45 (m, 1H), δ7.51-7.53 (d, 1H), δ7.63-7.75 (m, 2H), δ8.14 (s, 1H), δ8.32 (s, 1H), δ10.95 (s, 1H).

### Example 210. Compound 212

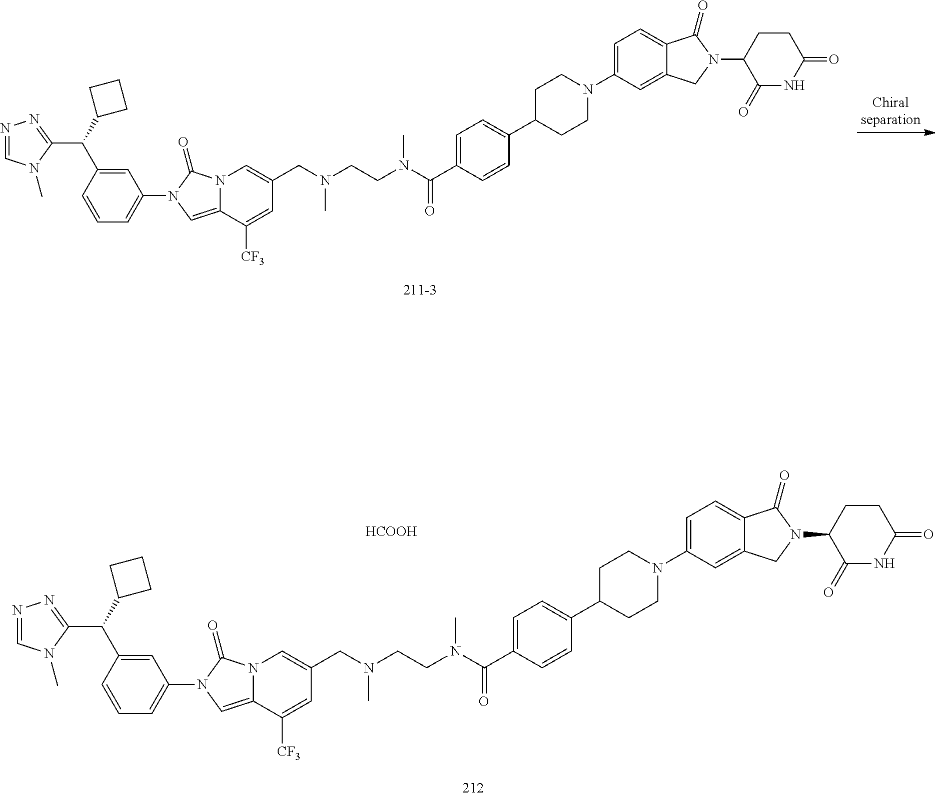 [SMILES: CN(CCN(C)C(=O)c1ccc(C2CCN(c3ccc4c(c3)CN(C3CCC(=O)NC3=O)C4=O)CC2)cc1)Cc1cc(C(F)(F)F)c2cn(-c3cccc([C@H](c4nncn4C)C4CCC4)c3)c(=O)n2c1 ; CN(CCN(C)C(=O)c1ccc(C2CCN(c3ccc4c(c3)CN([C@H]3CCC(=O)NC3=O)C4=O)CC2)cc1)Cc1cc(C(F)(F)F)c2cn(-c3cccc([C@H](c4nncn4C)C4CCC4)c3)c(=O)n2c1 ; "HCOOH"]

### Synthesis of Compound 212.

The Compound 211-3 (247 mg) was purified by Prep-CHIRAL-HPLC with the following conditions (Column: CHIRALPAK IH, 2*25 cm, 5 μm; Mobile Phase A: Hex (0.2% TEA)—HPLC, Mobile Phase B: IPA:DCM=1:1—HPLC; Flow rate: 20 mL/min; Gradient: 70% B to 70% B in 16 min; Wave Length: 220/254 nm; RT1 (min): 11.51; RT2 (min): 12.75; Sample Solvent: IPA:DCM=1:1—HPLC; Injection Volume: 0.85 mL; Number Of Runs: 10; Number Of Runs: 0) The second peak was the product. The residue was purified by reversed-phase flash chromatography with the following conditions: column, C18 silica gel; mobile phase, MeCN in water (0.1% FA), 10% to 50% gradient in 30 min; detector, UV 254 nm. This resulted in Compound 212 (25.3 mg, 4.45%) as a yellow solid.

LCMS-Compound 212: (ES, m/z): [M-COOH]⁺ 957

NMR-Compound 212: (400 MHz, DMSO-d6, ppm): δ 1.70-1.80 (m, 10H), δ1.90-2.01 (m, 3H), δ2.07-2.08 (d, 1H), δ2.25 (s, 2H), δ2.32-2.39 (m, 2H), δ2.50-2.61 (m, 2H), δ2.66-2.67 (m, 1H), δ2.85-2.95 (m, 6H), δ3.12-3.20 (m, 3H), δ3.42 (s, 4H), δ3.59 (s, 1H), δ3.99 (s, 2H), δ4.19-4.35 (m, 3H), δ5.03-5.07 (m, 1H), δ7.03-7.11 (m, 3H), δ7.17-7.19 (d, 1H), δ7.27-7.32 (m, 5H), δ7.41-7.45 (m, 1H), δ7.51-7.53 (d, 1H), δ7.63-7.75 (m, 2H), 88.14 (s, 1H), 68.32 (s, 1H), δ10.95 (s, 1H).

### Example 211. Compound 213

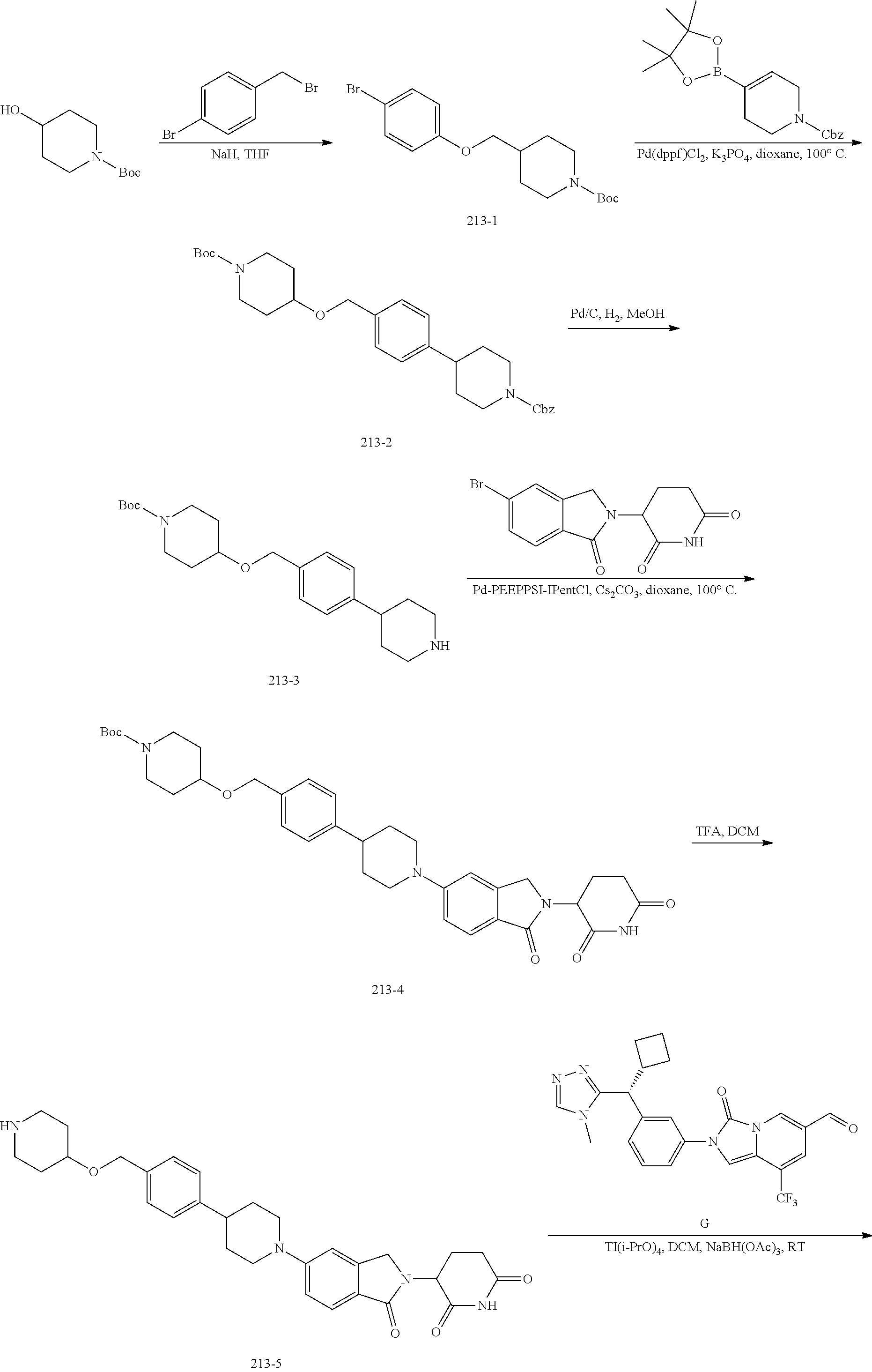

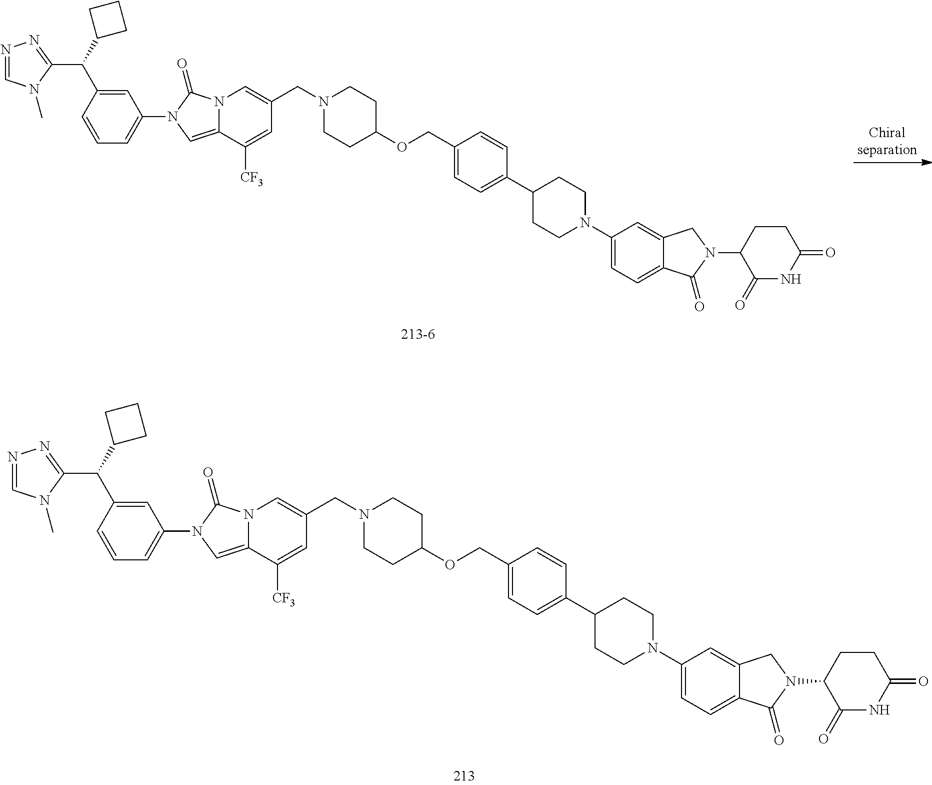 [SMILES: Cn1cnnc1[C@@H](c1cccc(-n2cc3c(C(F)(F)F)cc(CN4CCC(OCc5ccc(C6CCN(c7ccc8c(c7)CN(C7CCC(=O)NC7=O)C8=O)CC6)cc5)CC4)cn3c2=O)c1)C1CCC1 ; Cn1cnnc1[C@@H](c1cccc(-n2cc3c(C(F)(F)F)cc(CN4CCC(OCc5ccc(C6CCN(c7ccc8c(c7)CN([C@@H]7CCC(=O)NC7=O)C8=O)CC6)cc5)CC4)cn3c2=O)c1)C1CCC1]

### Synthesis of Compound 213-1.

To a stirred solution of tert-butyl 4-hydroxypiperidine-1-carboxylate (25 g, 124.214 mmol, 1 equiv) in THF (300 mL) was added NaH (9.94 g, 248.428 mmol, 2 equiv, 60%) at room temperature. The mixture was stirred for 1 h at room temperature. To the above mixture was added 1-bromo-4-(bromomethyl)benzene (34.15 g, 136.635 mmol, 1.1 equiv) at room temperature. The resulting mixture was stirred for 3 h at room temperature. The reaction was quenched with NH₄Cl (aq.) (1 L) at room temperature. The aqueous layer was extracted with EtOAc (3×400 mL). The resulting mixture was concentrated under vacuum. The residue was purified by silica gel column chromatography, eluted with PE/EA (20:1) to afford Compound 213-1 (40 g, 82.62%) as a white solid.

### Synthesis of Compound 213-2.

Into a 1 L 3-necked round-bottom flask were added Compound 213-1 (20 g, 54.012 mmol, 1 equiv), benzyl 4-(4,4,5,5-tetramethyl-1,3,2-dioxaborolan-2-yl)-3,6-dihydro-2H-pyridine-1-carboxylate (22.25 g, 64.814 mmol, 1.2 equiv), Pd(dppf)Cl₂ (1.98 g, 2.701 mmol, 0.05 equiv), K₃PO₄ (22.93 g, 108.024 mmol, 2 equiv) and dioxane (300 mL) at room temperature. The resulting mixture was stirred overnight at 100° C. under nitrogen atmosphere. The resulting mixture was concentrated under vacuum. The residue was purified by silica gel column chromatography, eluted with PE/EA (10:1) to afford Compound 213-2 (25 g, 91.36%) as a yellow oil.

### Synthesis of Compound 213-3.

To a solution of Compound 213-2 (12 g, 23.685 mmol, 1 equiv) in MeOH (250 mL) was added Pd/C (10%, 1.2 g) under nitrogen atmosphere in a 500 mL round-bottom flask. The mixture was hydrogenated at room temperature for 3 h under hydrogen atmosphere using a hydrogen balloon, filtered through a Celite pad and concentrated under reduced pressure. The resulting mixture was concentrated under vacuum. This resulted in Compound 213-3 (8.5 g, 95.82%) as a white oil.

### Synthesis of Compound 213-4.

Into a 250 mL 3-necked round-bottom flask were added Compound 213-3 (8.5 g, 22.695 mmol, 1 equiv), 3-(5-bromo-1-oxo-3H-isoindol-2-yl)piperidine-2,6-dione (11.00 g, 34.043 mmol, 1.5 equiv), Pd-PEPPSI-IPentCl 2-methylpyridine (o-picoline) (0.95 g, 1.135 mmol, 0.05 equiv), Cs₂CO₃ (14.79 g, 45.390 mmol, 2 equiv) and dioxane (100 mL) at room temperature. The resulting mixture was stirred overnight at 100° C. under nitrogen atmosphere. The reaction was quenched by the addition of H₂O/AcOH (10:1) (400 mL) at room temperature. The aqueous layer was extracted with EtOAc (3×200 mL). The resulting mixture was concentrated under vacuum. The residue was purified by silica gel column chromatography, eluted with DCM/MeOH (50:1) to afford Compound 213-4 (5 g, 33.93%) as a brown solid.

### Synthesis of Compound 213-5.

To a stirred solution of Compound 213-4 (5 g, 8.107 mmol, 1 equiv) in DCM (40 mL) was added TFA (10 mL) at room temperature. The resulting mixture was stirred for 2 h at room temperature. The resulting mixture was diluted with DCM (100 mL). The resulting mixture was concentrated under vacuum. The residue was purified by reversed-phase flash chromatography with the following conditions: column, C18 silica gel; mobile phase, MeCN in water (0.1% TFA), 0% to 100% gradient in 30 min; detector, UV 220 nm. This resulted in Compound 213-5 (2.5 g, 59.69%) as a brown solid.

### Synthesis of Compound 213-6.

To a stirred solution of Compound 213-5 (1.2 g, 2.323 mmol, 1 equiv) and Intermediate G (1.06 g, 2.323 mmol, 1 equiv) in DCM (30 mL) was added Ti(Oi-Pr)₄ (1.98 g, 6.969 mmol, 3 equiv) at room temperature. The resulting mixture was stirred overnight at room temperature. To the above mixture was added STAB (1.97 g, 9.292 mmol, 4 equiv) at room temperature. The resulting mixture was stirred for additional 4 h at room temperature. The reaction was quenched with 10% HOAc (aq.) at room temperature. The aqueous layer was extracted with DCM (3×50 mL). The resulting mixture was concentrated under vacuum. The residue was purified by reversed-phase flash chromatography with the following conditions (column, C18 silica gel; mobile phase, MeCN in water (0.1% FA), 10% to 50% gradient in 30 min; detector, UV 254 nm) to afford crude product. The crude product was purified by Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 20% B to 30% B in 10 min; Wave Length: 254; 220 nm; RT1 (min): 9.65) to afford Compound 213-6 (200 mg, 8.56%) as a yellow solid.

### Synthesis of Compound 213.

The Compound 213-6 (200 mg) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 60% B to 60% B in 12 min; Wave Length: 220/254 nm; RT1 (min): 6.92; RT2 (min): 9.83; the first peak is product) to afford the crude product. The residue was purified by reverse flash chromatography with the following conditions (column, C18 silica gel; mobile phase, MeCN in water (0.1% FA), 0% to 40% gradient in 10 min; detector, UV 254 nm) to afford Compound 213 (61.8 mg, 29.60%) as a yellow solid.

LC-MS-Compound 213: (ES, m/z): [M+H]⁺ 956

H-NMR-Compound 213: (400 MHz, DMSO-d₆, δ ppm): 1.44-1.61 (m, 2H), 1.65-1.91 (m, 11H), 1.92-2.22 (m, 4H), 2.31-2.45 (m, 1H), 2.53-2.83 (m, 4H), 2.85-2.95 (m, 3H), 3.15-3.28 (m, 3H), 3.39-3.42 (m, 4H), 4.00-4.03 (m, 2H), 4.18-4.35 (m, 3H), 4.45 (s, 2H), 5.03-5.07 (d, 1H), 7.01-7.15 (m, 3H), 7.18-7.31 (m, 6H), 7.41-7.45 (m, 1H), 7.51-7.53 (d, 1H), 7.66-7.73 (m, 3H), 8.33 (s, 1H), 10.95 (s, 1H).

### Example 212. Compound 214

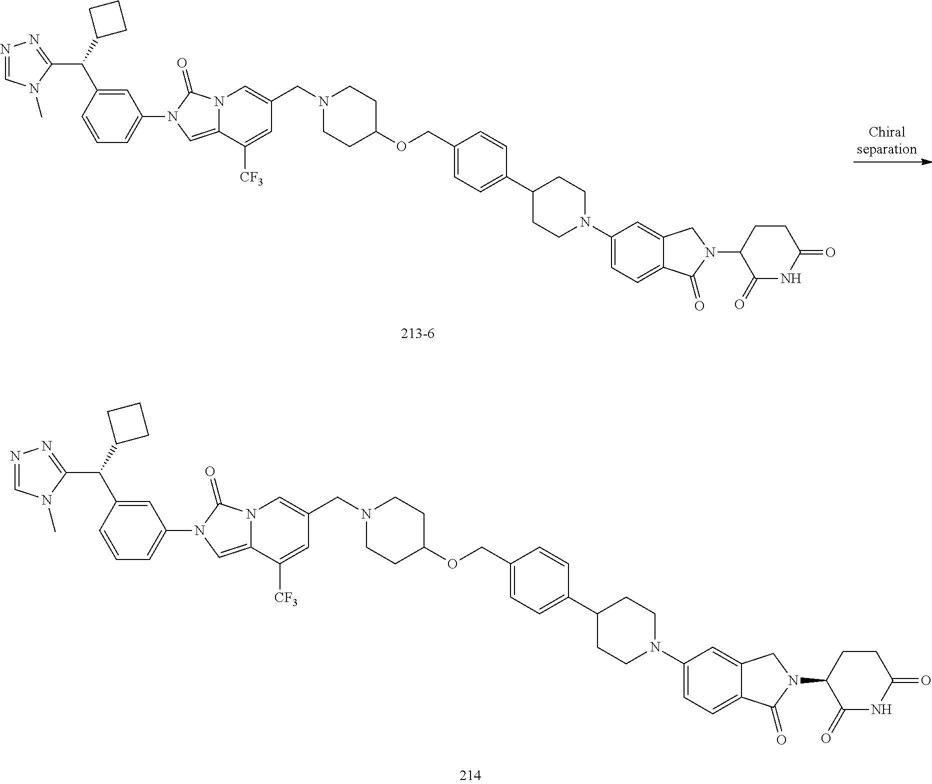 [SMILES: Cn1cnnc1[C@@H](c1cccc(-n2cc3c(C(F)(F)F)cc(CN4CCC(OCc5ccc(C6CCN(c7ccc8c(c7)CN(C7CCC(=O)NC7=O)C8=O)CC6)cc5)CC4)cn3c2=O)c1)C1CCC1 ; Cn1cnnc1[C@@H](c1cccc(-n2cc3c(C(F)(F)F)cc(CN4CCC(OCc5ccc(C6CCN(c7ccc8c(c7)CN([C@H]7CCC(=O)NC7=O)C8=O)CC6)cc5)CC4)cn3c2=O)c1)C1CCC1]

### Synthesis of Compound 214.

The Compound 213-6 (200 mg) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 60% B to 60% B in 12 min; Wave Length: 220/254 nm; RT1 (min): 6.92; RT2 (min): 9.83; the second peak is product) to afford the crude product. The crude product was purified by Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 25% B to 39% B in 7 min, 39% B; Wave Length: 254; 220 nm; RT1 (min): 6.5) to afford Compound 214 (44.0 mg, 21.56%) as a yellow solid.

LC-MS-Compound 214: (ES, m/z): [M+H]⁺ 956

H-NMR-Compound 214: (400 MHz, DMSO-d₆, δ ppm): 1.44-1.61 (m, 2H), 1.65-1.91 (m, 11H), 1.92-2.02 (m, 1H), 2.02-2.22 (m, 3H), 2.31-2.45 (m, 1H), 2.53-2.83 (m, 4H), 2.85-2.95 (m, 3H), 3.15-3.28 (m, 3H), 3.39-3.42 (m, 4H), 4.00-4.03 (m, 2H), 4.18-4.35 (m, 3H), 4.45 (s, 2H), 5.03-5.07 (d, 1H), 7.01 (s, 1H), 7.09-7.31 (m, 8H), 7.41-7.45 (m, 1H), 7.51-7.53 (d, 1H), 7.66-7.73 (m, 3H), 8.33 (s, 1H), 10.95 (s, 1H).

### Example 213. Compound 215

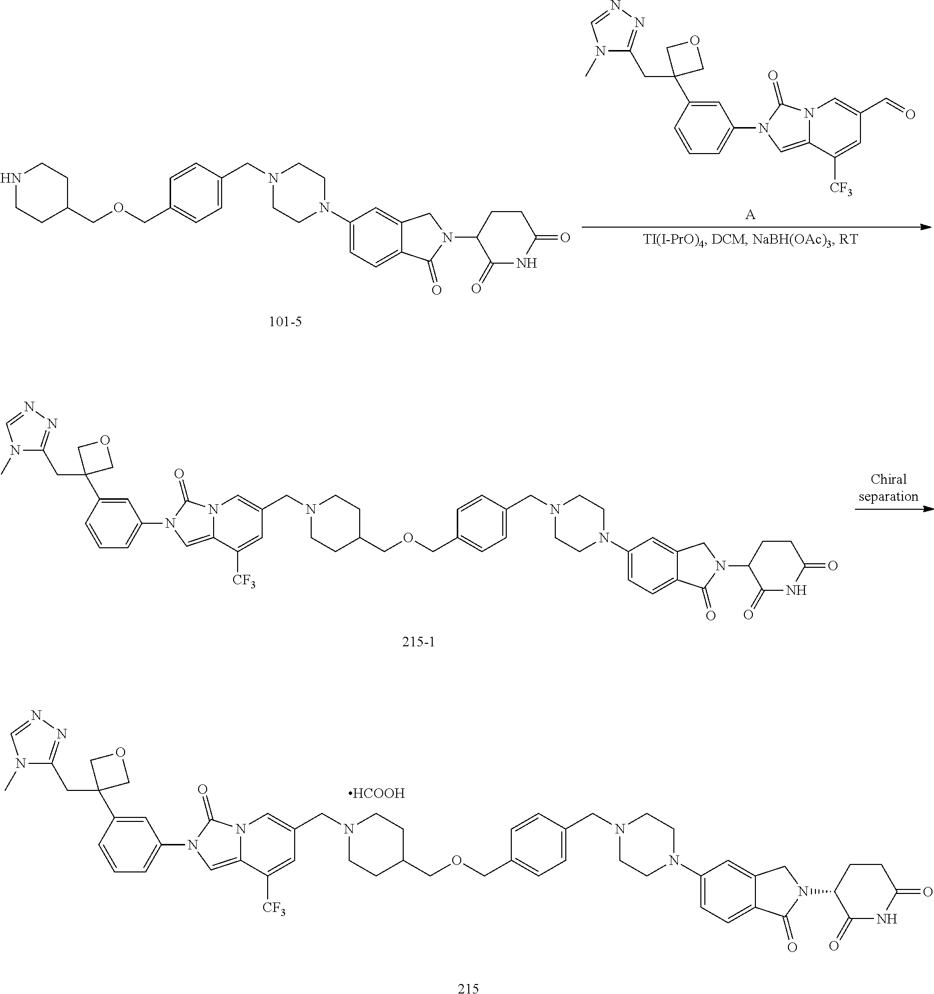

### Synthesis of Compound 215-1.

To a stirred solution of Compound 101-5 (1.2 g, 2.199 mmol, 1 equiv) and Intermediate A (1.01 g, 2.208 mmol, 1.00 equiv) in DCM (30 mL) was added Ti(Oi-Pr)₄ (1.88 g, 6.597 mmol, 3 equiv) at room temperature. The resulting mixture was stirred overnight at room temperature. To the above mixture was added NaBH(OAc)₃ (1.40 g, 6.597 mmol, 3 equiv) at room temperature. The resulting mixture was stirred for additional 6 h at mom temperature. The reaction was quenched with 10% HOAc (aq.) at room temperature. The aqueous layer was extracted with EtOAc (50 mL). The aqueous layer was filtered, the filter cake was washed with water (10 mL). The filtrate was concentrated under reduced pressure. The residue was purified by reverse flash chromatography with the following conditions (column, C18 silica gel; mobile phase, MeCN in Water (0.1% FA), 10% to 50% gradient in 20 min; detector, UV 254 nm) to afford the crude product. The crude product was purified by Prep-HPLC with the following conditions (Column. XSelect CSH Fluoro Phenyl, 30*150 mm, 5 μm; Mobile Phase A: Water (0.1% FA), Mobile Phase B: ACN; Flow rate: 60 mL/min; Gradient: 8% B to 20% B in 8 min, Wave Length: 254; 220 nm; RT1 (min): 7.98) to afford Compound 215-1 (150 mg, 6.70%) as a yellow solid.

### Synthesis of Compound 215.

The Compound 215-1 (150 mg) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 90% B to 90% B in 12 min; Wave Length: 220/254 nm; RT1 (min): 6.03; RT2 (min): 9.72, the first peak is product) to afford the crude product. The crude product was purified by Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 10% B to 21% B in 7 min, Wave Length: 254; 220 nm; RT1 (min): 6.03) to afford Compound 215 (37.9 mg, 23.37%) as a yellow solid.

LC-MS-Compound 215: (ES, m/z): [M−HCOOH+H]⁺ 987

H-NMR-Compound 215: (400 MHz, DMSO-d₆, δ ppm): 1.21-1.44 (m, 2H), 1.61-1.83 (m, 3H), 1.91-2.01 (m, 1H), 2.31-2.45 (m, 2H), 2.50-2.67 (m, 6H), 2.88-2.95 (m, 1H), 2.98 (s, 3H), 3.21-3.28 (m, 10H), 3.53-3.56 (m, 4H), 4.17-4.21 (m, 1H), 4.30-4.34 (m, 1H), 4.45 (s, 2H), 4.90-4.95 (m, 4H), 5.02-5.07 (d, 1H), 6.91-6.93 (d, 1H), 7.03-7.09 (m, 3H), 7.28-7.42 (m, 7H), 7.50-7.52 (d, 1H), 7.73-7.76 (d, 1H), 7.85 (s, 1H), 8.14 (s, 1H), 8.20 (s, 1H), 10.95 (s, 1H).

### Example 214. Compound 216

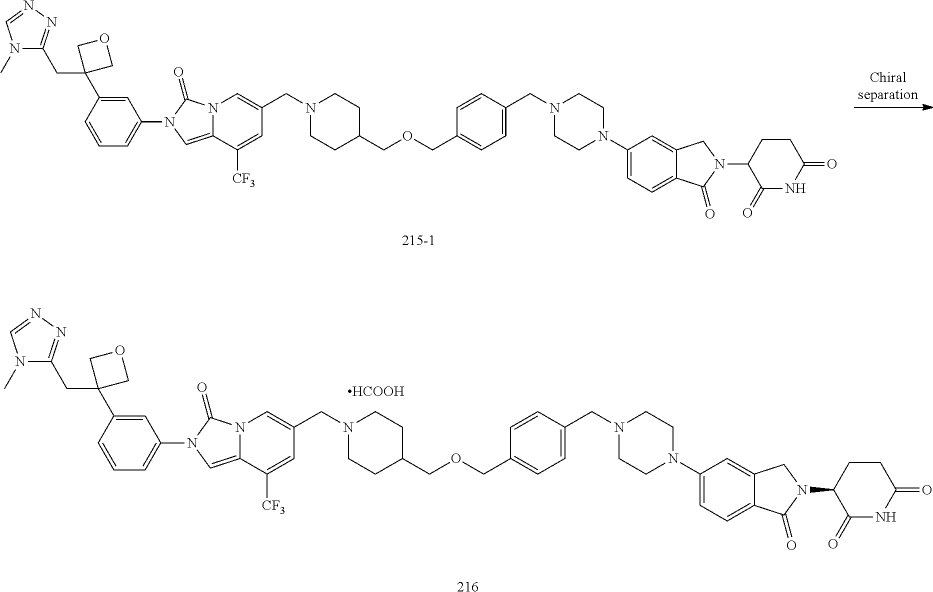 [SMILES: Cn1cnnc1CC1(c2cccc(-n3cc4c(C(F)(F)F)cc(CN5CCC(COCc6ccc(CN7CCN(c8ccc9c(c8)CN(C8CCC(=O)NC8=O)C9=O)CC7)cc6)CC5)cn4c3=O)c2)COC1 ; "215-1" ; Cn1cnnc1CC1(c2cccc(-n3cc4c(C(F)(F)F)cc(CN5CCC(COCc6ccc(CN7CCN(c8ccc9c(c8)CN([C@H]8CCC(=O)NC8=O)C9=O)CC7)cc6)CC5)cn4c3=O)c2)COC1 ; "·HCOOH" ; "216"]

### Synthesis of Compound 216.

The Compound 215-1 (150 mg) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 90% B to 90% B in 12 min; Wave Length: 220/254 nm; RT1 (min): 6.03; RT2 (min): 9.72, the second peak is product) to afford the crude product. The crude product was purified by Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 10% B to 21% B in 7 min, Wave Length: 254; 220 nm; RT1 (min): 6.03) to afford Compound 216 (36.3 mg, 22.59%) as a yellow solid.

LC-MS-Compound 216: (ES, m/z): [M−HCOOH+H]⁺ 987

H-NMR-Compound 216: (400 MHz, DMSO-d₆, δ ppm): 1.21-1.44 (m, 2H), 1.61-1.83 (m, 3H), 1.91-2.01 (m, 1H), 2.31-2.45 (m, 2H), 2.50-2.67 (m, 6H), 2.88-2.95 (m, 1H), 2.98 (s, 3H), 3.21-3.28 (m, 10H), 3.53-3.56 (m, 4H), 4.17-4.21 (m, 1H), 4.30-4.34 (m, 1H), 4.45 (s, 2H), 4.90-4.95 (m, 4H), 5.02-5.07 (d, 1H), 6.91-6.93 (d, 1H), 7.03-7.09 (m, 3H), 7.28-7.42 (m, 7H), 7.50-7.52 (d, 1H), 7.73-7.76 (d, 1H), 7.85 (s, 1H), 8.14 (s, 1H), 8.20 (s, 1H), 10.95 (s, 1H).

### Example 215. Compound 217

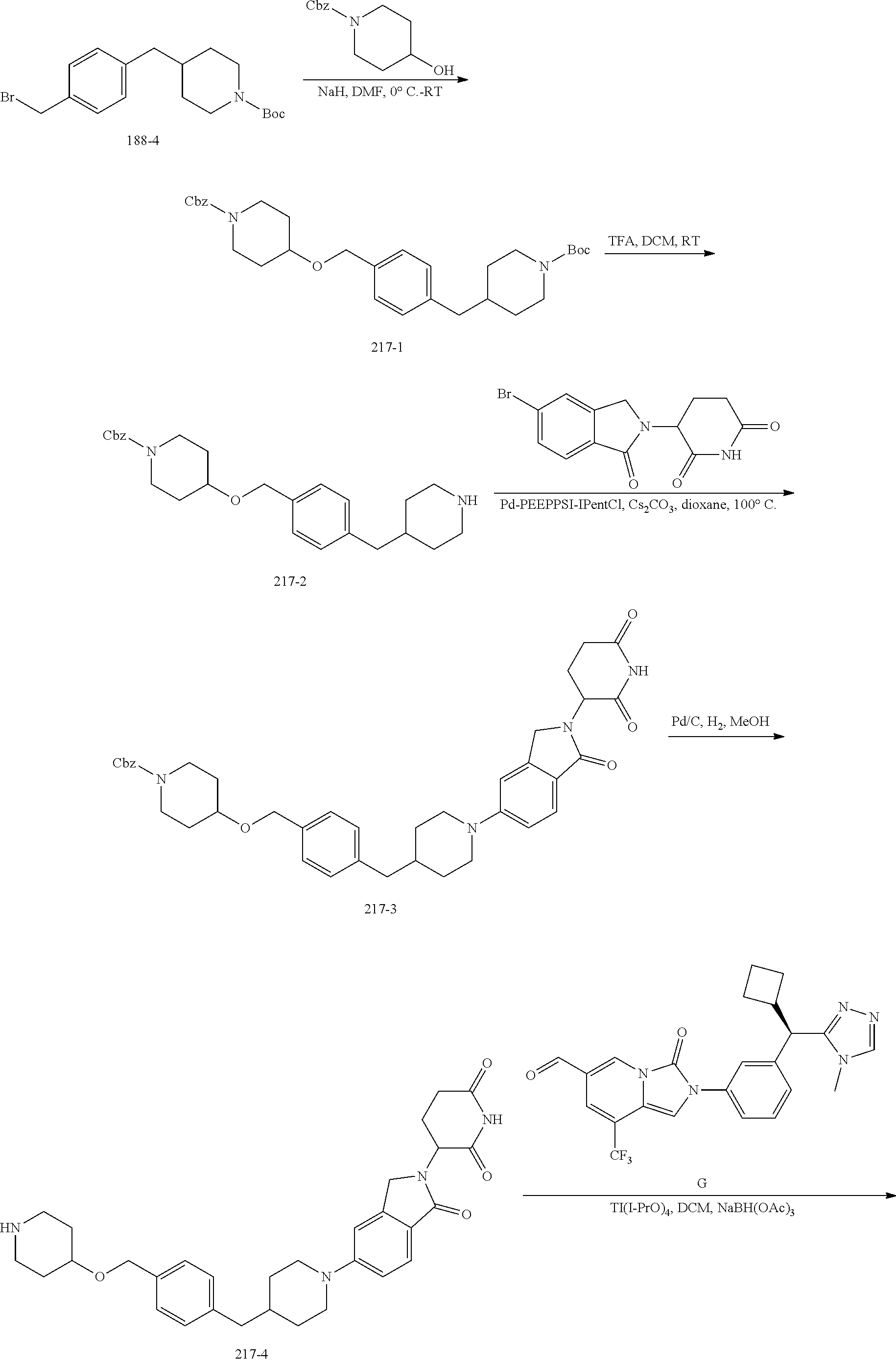

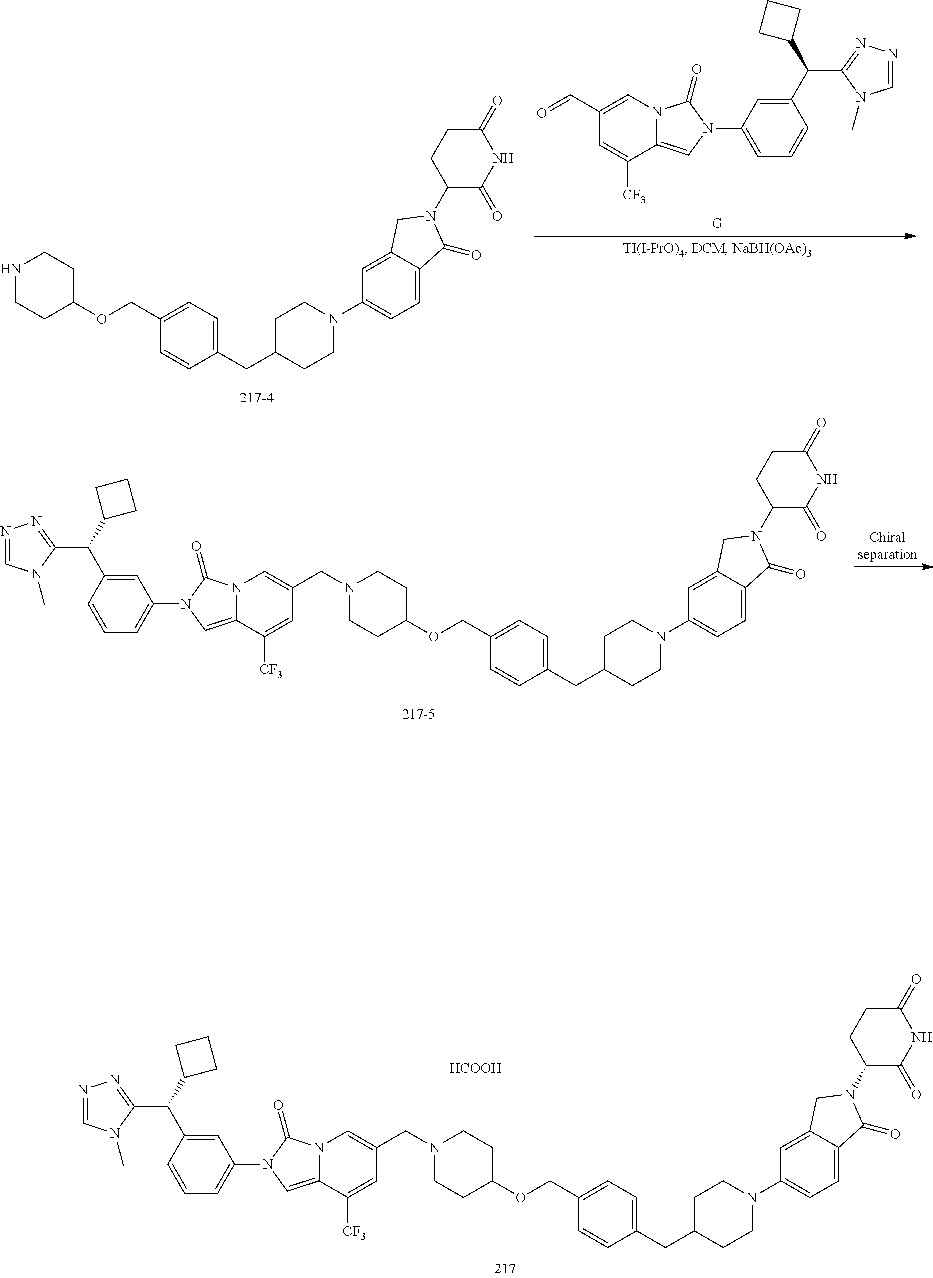

### Synthesis of Compound 217-1.

A solution of benzyl 4-hydroxypiperidine-1-carboxylate (3.8 g, 16.29 mmol, 1 equiv) in THF (60 mL) was treated with NaH (0.6 g, 24.43 mmol, 1.5 equiv) at 0° C. under nitrogen atmosphere followed by the addition of Compound 188-4 (6 g, 16.29 mmol, 1.0 equiv) at room temperature. The resulting mixture was stirred for overnight at room temperature. The reaction was quenched by the addition of water (200 mL) at room temperature. The aqueous layer was extracted with EtOAc (3×60 mL). The resulting mixture was concentrated under reduced pressure. The residue was purified by silica gel column chromatography, eluted with PE/EA (5:1) to afford Compound 217-1 (6 g, 65%) as a colorless oil.

### Synthesis of Compound 217-2.

Into a 100 mL, round-bottom flask were added TFA (10 mL) and DCM (30 mL) at room temperature. To the above mixture was added Compound 217-1 (4 g, 7.65 mmol, 1.0 equiv) at room temperature. The resulting mixture was stirred for additional 3 h at room temperature. The resulting mixture was diluted with CH₂Cl₂ (20 mL). The resulting mixture was concentrated under reduced pressure. The mixture was neutralized to pH 7 with saturated NaHCO₃ (aq.). The residue was purified by silica gel column chromatography, eluted with CH₂Cl₂/MeOH (20:1) to afford Compound 217-2 (3 g, 88%) as a light yellow oil.

### Synthesis of Compound 217-3.

Into a 50 mL round-bottom flask were added Compound 217-2 (3 g, 7.09 mmol, 1.0 equiv) and 3-(5-bromo-1-oxo-3H-isoindol-2-yl) piperidine-2,6-dione (3 g, 9.22 mmol, 1.3 equiv) at room temperature. To the above mixture was added Cs₂CO₃ (6.9 g, 21.29 mmol, 3.0 equiv) and Pd-PEPPSI-IPentCl 2-methylpyridine (o-picoline (298 mg, 0.35 mmol, 0.05 equiv) in dioxane (30 mL) at room temperature. The resulting mixture was stirred for overnight at 100° C. under nitrogen atmosphere. The reaction was quenched by the addition of 10% AcOH (aq.) (200 mL) at room temperature. The aqueous layer was extracted with CH₂Cl₂/MeOH (10/1) (3×50 mL). The resulting mixture was concentrated under reduced pressure. The residue was purified by Prep-TLC (CH₂Cl₂/MeOH=30:1) to afford Compound 217-3 (1.6 g, 31%) as a light yellow solid.

### Synthesis of Compound 217-4.

To a stirred solution of Compound 217-3 (1.2 g) in MeOH (30 mL) was added Pd/C (0.15 g) at room temperature under nitrogen atmosphere. The resulting mixture was stirred for 15 min at room temperature under hydrogen atmosphere. The resulting mixture was filtered; the filter cake was washed with MeOH (3×10 mL). The filtrate was concentrated under reduced pressure. The residue was purified by reversed-phase flash chromatography with the following conditions: column, C18 silica gel; mobile phase, MeCN in water (0.1% FA), 10% to 50% gradient in 10 min; detector, UV 254 nm. To afford Compound 217-4 (500 mg, 49%) as a light yellow oil.

### Synthesis of Compound 217-5.

To a stirred solution of Compound 217-4 (980 mg, 1.84 mmol, 1.0 equiv) and Intermediate G (1 g, 2.21 mmol, 1.2 equiv) in DCM (10 mL) was added Ti(Oi-Pr)₄ (2.1 g, 7.38 mmol, 4.0 equiv) at room temperature. The resulting mixture was stirred for overnight at room temperature. To the above mixture was added NaBH(OAc)₃ (1.1 g, 5.54 mmol, 3.0 equiv) at room temperature. The resulting mixture was stirred for additional 2 h at room temperature. The reaction was quenched by the addition of 10% AcOH (100 mL) at room temperature. The aqueous layer was extracted with CH₂Cl₂:MeOH (3×100 mL). The resulting mixture was concentrated under reduced pressure. The residue was purified by reversed-phase flash chromatography with the following conditions: column, C18 silica gel; mobile phase, MeCN in water (0.1% FA), 10% to 50% gradient in 30 min; detector, UV 254 nm. The crude product (650 mg) was purified by Prep-HPLC with the following conditions (Column: Sunfire prep C18 column, 30*150 mm, 5 μm; Mobile Phase A: Water (0.1% FA), Mobile Phase B: ACN; Flow rate: 60 mL/min; Gradient: 27% B to 38% B in 8 min, 38% B; Wave Length: 254; 220 nm; RT1 (min): 7.18; Number of Runs: 0) to afford Compound 217-5 (350 mg, 20%) as a yellow solid.

### Synthesis of Compound 217.

The Compound 217-5 (380 mg, 0.39 mmol, 1.0 equiv) was purified by Prep-CHIRAL-HPLC with the following conditions (Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 70% B to 70% B in 10 min; Wave Length: 220/254 nm; RT1 (min): 5.29; RT2 (min): 8.01; Sample Solvent: DCM:ACN=1:1—HPLC; Injection Volume: 0.7 mL; Number Of Runs: 7), The first peak was the product. The residue was purified by reversed-phase flash chromatography with the following conditions: column, C18 silica gel; mobile phase, MeCN in water (0.1% FA), 10% to 50% gradient in 30 min; detector, UV 254 nm. This resulted in Compound 217 (142 mg, 36%) as a yellow solid.

LC-MS-Compound 217: (ES, m/z): [M-COOH]⁺ 970

H-NMR-Compound 217: (400 MHz, DMSO-d6, δ ppm): 1.23-1.26 (m, 2H), 1.52-1.55 (m, 2H), 1.61-1.65 (m, 2H), 1.72-1.90 (m, 8H), 2.03-2.15 (m, 3H), 2.38-2.49 (m, 1H), 2.55-2.59 (m, 2H), 2.68-2.75 (m, 4H), 2.83-2.90 (m, 1H), 3.15-3.20 (m, 4H), 3.28-3.32 (m, 4H), 3.84-3.87 (m, 2H), 4.16-4.32 (m, 3H), 4.45 (s, 2H), 5.02-5.06 (m, 1H), 7.01-7.02 (m, 3H), 7.14-7.24 (m, 5H), 7.30 (s, 1H), 7.41-7.49 (m, 2H), 7.66-7.73 (m, 3H), 8.14 (s, 1H), 8.32 (s, 1H), 10.95 (s, 1H).

### Example 216. Compound 218

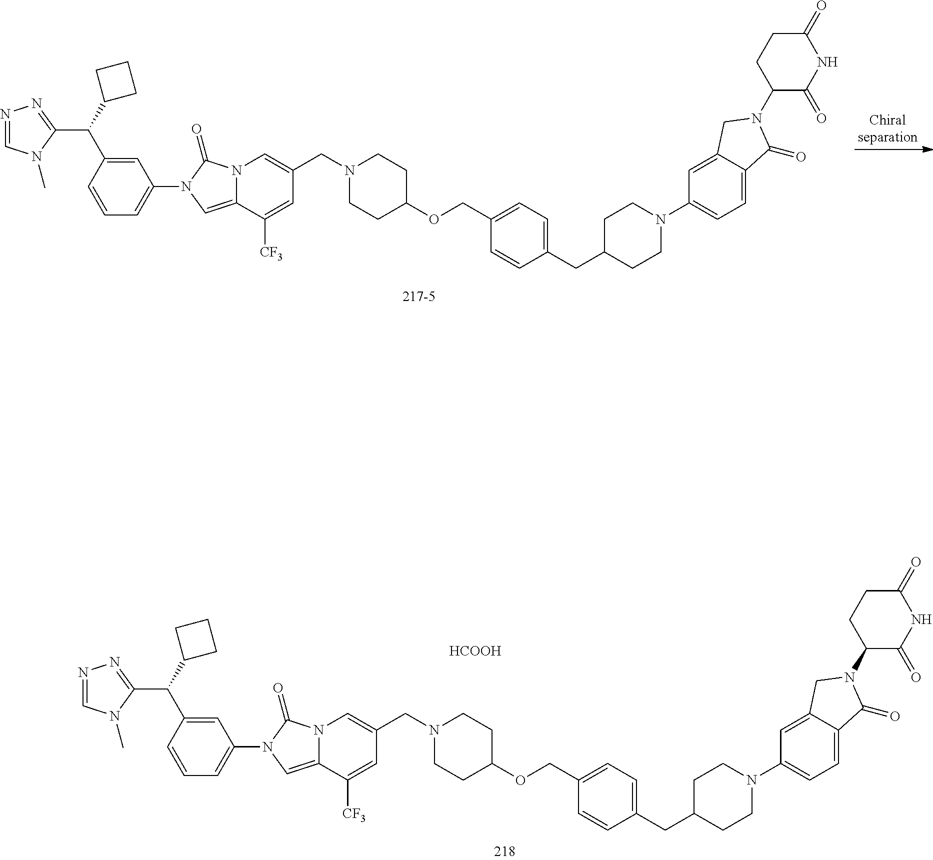 [SMILES: Cn1cnnc1[C@@H](c1cccc(-n2cc3c(C(F)(F)F)cc(CN4CCC(OCc5ccc(CC6CCN(c7ccc8c(c7)CN(C7CCC(=O)NC7=O)C8=O)CC6)cc5)CC4)cn3c2=O)c1)C1CCC1 ; Cn1cnnc1[C@@H](c1cccc(-n2cc3c(C(F)(F)F)cc(CN4CCC(OCc5ccc(CC6CCN(c7ccc8c(c7)CN([C@H]7CCC(=O)NC7=O)C8=O)CC6)cc5)CC4)cn3c2=O)c1)C1CCC1 ; "HCOOH"]

### Synthesis of Compound 218.

The Compound 217-5 (380 mg) was purified by Prep-CHIRAL-HPLC with the following conditions (Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 70% 13 to 70% B in 10 min; Wave Length: 220/254 nm; RT1 (min): 5.29; RT2 (min): 8.01; Sample Solvent: DCM:ACN=1:1—HPLC; Injection Volume: 0.7 mL; Number Of Runs: 7), The second peak was the product. The residue was purified by reversed-phase flash chromatography with the following conditions: column, (C18 silica gel; mobile phase, MeCN in water (0.1% FA), 10% to 50% gradient in 30 min; detector, UV 254 nm. This resulted in Compound 218 (122 mg, 31%) as a yellow solid.

LC-MS-Compound 218: (ES, m/z): [M-COOH]⁺ 970

H-NMR-Compound 218: (400 MHz, DMSO-d6, δ ppm): 1.23-1.26 (m, 2H), 1.52-1.55 (m, 2H), 1.61-1.65 (m, 2H), 1.72-1.90 (m, 8H), 2.03-2.15 (m, 3H), 2.38-2.49 (m, 1H), 2.55-2.59 (m, 2H), 2.68-2.75 (m, 4H), 2.83-2.90 (m, 1H), 3.15-3.20 (m, 4H), 3.28-3.32 (m, 4H), 3.84-3.87 (m, 2H), 4.16-4.32 (m, 3H), 4.45 (s, 2H), 5.02-5.06 (m, 1H), 7.01-7.02 (m, 3H), 7.14-7.24 (m, 5H), 7.30 (s, 1H), 7.41-7.49 (m, 2H), 7.66-7.73 (m, 3H), 8.14 (s, 1H), 8.32 (s, 1H), 10.95 (s, 1H).

### Example 217. Compound 219

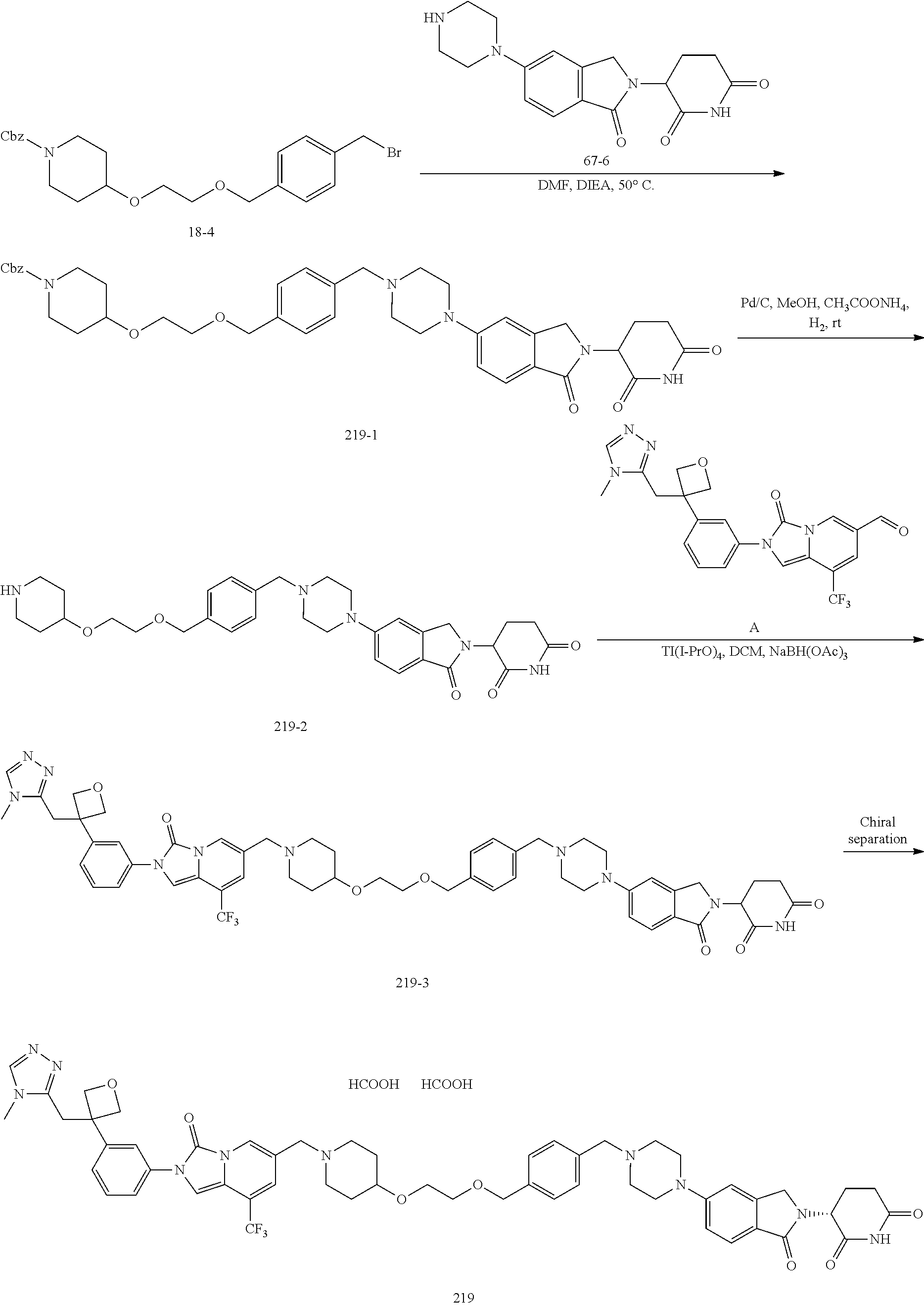

### Synthesis of Compound 219-1.

To a stirred solution of benzyl Compound 18-4 (5 g, 10.81 mmol, 1.0 equiv) and Compound 67-6 (5.3 g, 16.22 mmol, 1.5 equiv) in DMF (50 mL) was added DIEA (2.8 g, 21.62 mmol, 2.0 equiv) at room temperature. The resulting mixture was stirred for overnight at room temperature. The reaction was quenched by the addition of 10% AcOH (500 mL) at room temperature. The aqueous layer was extracted with CH₂Cl₂ (3×400 mL). The residue was purified by silica gel column chromatography, eluted with CH₂Cl₂/MeOH (20:1) to afford Compound 219-1 (5 g, 65%) as an off-white solid.

### Synthesis of Compound 219-2.

To a solution of Compound 219-1 (5 g, 7.04 mmol, 1.0 equiv) and CH₃COONH₄ (5.4 g, 70.44 mmol, 10.0 equiv) in 150 mL MeOH was added Pd/C (10%, 1.5 g) under nitrogen atmosphere in a 250 mL round-bottom flask. The mixture was hydrogenated at room temperature for 2 h under hydrogen atmosphere using a hydrogen balloon, filtered through a Celite pad and concentrated under reduced pressure. The resulting mixture was filtered; the filter cake was washed with DCM (3×100 mL). The filtrate was concentrated under reduced pressure. The residue was purified by reversed-phase flash chromatography with the following conditions: column, C18 silica gel; mobile phase, MeCN in water (0.1% FA), 10% to 50%, gradient in 30 min; detector, UV 254 nm. This resulted in Compound 219-2 (2.8 g, 69%) as an off-white solid.

### Synthesis of Compound 219-3.

To a stirred solution of Compound 219-2 (1 g, 1.73 mmol, 1.0 equiv) and Intermediate A (1 g, 2.08 mmol, 1.2 equiv) in DCM (10 mL) was added Ti(Oi-Pr)₄ (2 g, 6.94 mmol, 4.0 equiv) at room temperature. The resulting mixture was stirred for overnight at room temperature. To the above mixture was added NaBH(OAc)₃ (1.1 g, 5.21 mmol, 3.0 equiv) at room temperature. The resulting mixture was stirred for additional 2 h at room temperature. The reaction was quenched by the addition of 10% AcOH (100 mL) at room temperature. The aqueous layer was extracted with CH₂Cl₂:MeOH (10:1) (3×100 mL). The residue was purified by reversed-phase flash chromatography with the following conditions: column, C18 silica gel; mobile phase, MeCN in water (0.1% FA), 10% to 50% gradient in 30 min; detector, UV 254 nm. The crude product (600 mg) was purified by Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 5% B to 19% B in 11 min, 19% B; Wave Length: 254; 220 nm; RT1 (min): 10.65; Number of Runs: 0) to afford Compound 219-3 (420 mg, 22%) as a yellow solid.

### Synthesis of Compound 219.

The Compound 219-3 (420 mg) was purified by Prep-CHIRAL-HPLC with the following conditions Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 90% B to 90% B in 10 min; Wave Length: 220/254 nm; RT1 (min): 5.40; RT2 (min): 8.01; Sample Solvent: DCM:ACN=1:1—HPLC; Injection Volume: 0.9 mL; Number Of Runs: 6. The first peak was the product. The residue was purified by reversed-phase flash chromatography with the following conditions: column, C18 silica gel; mobile phase, MeCN in water (0.1% FA), 10% to 50% gradient in 30 min; detector, UV 254 nm. This resulted in Compound 219 (158 mg, 35%) as a yellow solid.

LC-MS-Compound 219: (ES, m/z): [M−2HCOOH]⁺ 1017

H-NMR-Compound 219: (400 MHz, DMSO-d6, δ ppm): 1.44-1.46 (m, 2H), 1.81-1.83 (m, 2H), 1.90-1.92 (m, 1H), 2.14-2.16 (m, 2H), 2.31-2.35 (m, 1H), 2.55-2.71 (m, 4H), 2.90-2.93 (m, 1H), 2.96 (s, 3H), 3.28-3.35 (m, 8H), 3.52-3.55 (m, 9H), 4.17-4.21 (d, 1H), 4.29-4.33 (d, 1H), 4.48 (s, 2H), 4.90-4.95 (m, 4H), 5.01-5.06 (m, 1H), 6.88-6.90 (d, 1H), 7.01-7.04 (m, 3H), 7.28-7.32 (m, 5H), 7.37-7.41 (m, 2H), 7.49-7.51 (d, 1H), 7.67 (s, 1H), 7.73-7.75 (d, 1H), 8.14 (s, 2H), 8.19 (s, 1H), 10.95 (s, 1H).

### Example 218. Compound 220

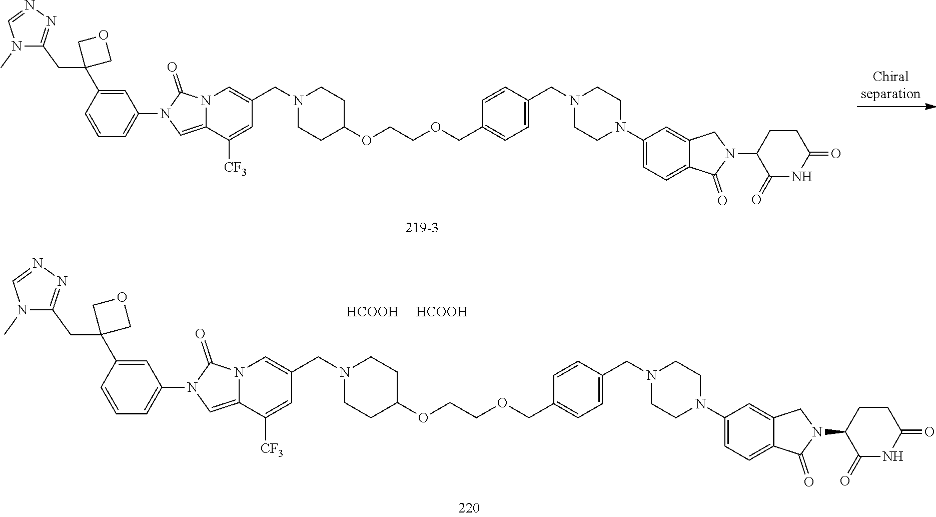 [SMILES: Cn1cnnc1CC1(c2cccc(-n3cc4c(C(F)(F)F)cc(CN5CCC(OCCOCc6ccc(CN7CCN(c8ccc9c(c8)CN(C8CCC(=O)NC8=O)C9=O)CC7)cc6)CC5)cn4c3=O)c2)COC1 ; Cn1cnnc1CC1(c2cccc(-n3cc4c(C(F)(F)F)cc(CN5CCC(OCCOCc6ccc(CN7CCN(c8ccc9c(c8)CN([C@H]8CCC(=O)NC8=O)C9=O)CC7)cc6)CC5)cn4c3=O)c2)COC1 ; "HCOOH" ; "HCOOH"]

### Synthesis of Compound 220.

The Compound 219-3 (420 mg) was purified by Prep-CHIRAL-HPLC with the following conditions Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 90% B to 90% B in 10 min; Wave Length: 220/254 nm; RT1 (min): 5.40; RT2 (min): 8.01; Sample Solvent: DCM:ACN=1:1—HPLC; Injection Volume: 0.9 mL; Number Of Runs: 6. The second peak was the product. The residue was purified by reversed-phase flash chromatography with the following conditions: column, C18 silica gel; mobile phase, MeCN in water (0.1% FA), 10% to 50% gradient in 30 min; detector, UV 254 nm. This resulted in Compound 220 (125 mg, 28%) as a yellow solid.

LC-MS-Compound 220: (ES, m/z): [M−2HCOOH]⁺ 1017

H-NMR-Compound 220: (400 MHz, DMSO-d6, δ ppm): 1.44-1.46 (m, 2H), 1.81-1.83 (m, 2H), 1.90-1.92 (m, 1H), 2.14-2.16 (m, 2H), 2.31-2.35 (m, 1H), 2.55-2.71 (m, 4H), 2.90-2.93 (m, 1H), 2.96 (s, 3H), 3.28-3.35 (m, 8H), 3.52-3.55 (m, 9H), 4.17-4.21 (d, 1H), 4.29-4.33 (d, 1H), 4.48 (s, 2H), 4.90-4.95 (m, 4H), 5.01-5.06 (m, 1H), 6.88-6.90 (d, 1H), 7.01-7.04 (m, 3H), 7.28-7.32 (m, 5H), 7.37-7.41 (m, 2H), 7.49-7.51 (d, 1H), 7.67 (s, 1H), 7.73-7.75 (d, 1H), 8.14 (s, 2H), 8.19 (s, 1H), 10.95 (s, 1H).

### Example 219. Compound 221

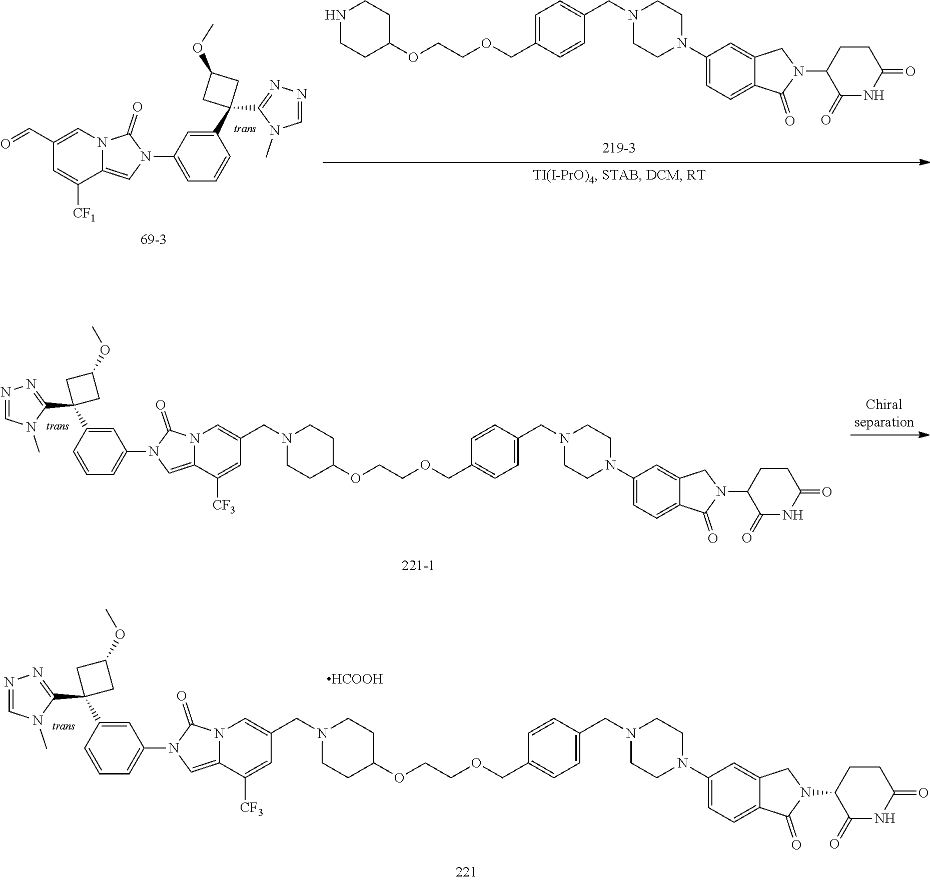

### Synthesis of Compound 221-1.

To a stirred solution of Compound 69-3 (1 g, 2.121 mmol, 1 equiv) and Compound 219-3 (1.22 g, 2.121 mmol, 1 equiv) in DCM (30 mL) was added Ti(Oi-Pr)₄ (1.81 g, 6.363 mmol, 3 equiv) at room temperature. The resulting mixture was stirred overnight at room temperature. To the above mixture was added STAB (1.35 g, 6.363 mmol, 3 equiv) at room temperature. The resulting mixture was stirred for additional 6 h at room temperature. The reaction was quenched with 10% HOAc (aq.) at room temperature. The aqueous layer was extracted with EtOAc (80 mL). The aqueous layer was filtered, the filter cake was washed with water (10 mL). The filtrate was concentrated under reduced pressure. The residue was purified by reverse flash chromatography with the following conditions: column, (C18 silica gel; mobile phase, MeCN in water (0.1% FA), 10% to 50% gradient in 20 min; detector, UV 254 nm. This resulted in Compound 221-1 (500 mg, 21.95%) as a yellow solid.

### Synthesis of Compound 221.

The Compound 221-1 (500 mg) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 75% B to 75% B in 8 min; Wave Length: 220/254 nm; RT1 (min): 4.09; RT2 (min): 5.73; the first peak is product) to afford the crude product. The residue was purified by reverse flash chromatography with the following conditions (column, C18 silica gel; mobile phase, MeCN in water (0.1% FA), 0% to 40% gradient in 10 min; detector, UV 254 nm) to afford Compound 221 (180.9 mg, 34.45%) as a yellow solid.

LC-MS-Compound 221: (ES, m/z): [M−HCOOH+H]⁺ 1031

H-NMR-Compound 221: (400 MHz, DMSO-d₆, δ ppm): 1.43-1.51 (m, 2H), 1.81-1.89 (m, 2H), 1.91-2.01 (m, 1H), 2.25-2.45 (m, 3H), 2.50-2.60 (m, 4H), 2.60-2.67 (m, 3H), 2.75-2.85 (m, 2H), 2.85-2.95 (m, 1H), 3.17 (s, 3H), 3.23 (s, 3H), 3.33-3.43 (m, 4H), 3.43-3.48 (m, 4H), 3.53-3.58 (m, 7H), 3.83-3.87 (m, 1H), 4.16-4.33 (m, 2H), 4.48 (s, 2H), 5.02-5.06 (d, 1H), 7.03-7.09 (m, 4H), 7.28-7.33 (m, 4H), 7.41-7.52 (m, 3H), 7.64-7.74 (m, 3H), 8.14 (s, 1H), 8.39 (s, 1H), 10.95 (s, 1H).

### Example 220. Compound 222

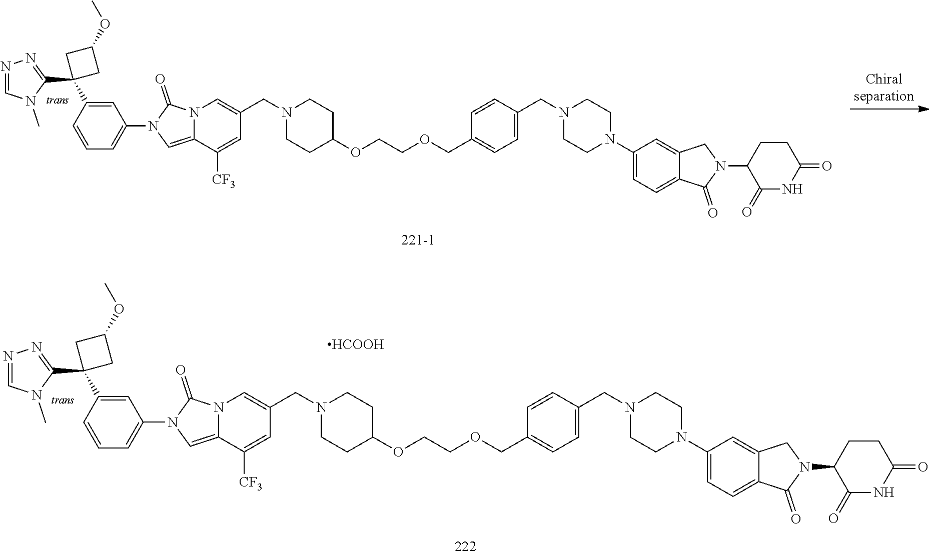 [SMILES: CO[C@H]1C[C@](c2cccc(-n3cc4c(C(F)(F)F)cc(CN5CCC(OCCOCc6ccc(CN7CCN(c8ccc9c(c8)CN(C8CCC(=O)NC8=O)C9=O)CC7)cc6)CC5)cn4c3=O)c2)(c2nncn2C)C1 ; CO[C@H]1C[C@](c2cccc(-n3cc4c(C(F)(F)F)cc(CN5CCC(OCCOCc6ccc(CN7CCN(c8ccc9c(c8)CN([C@H]8CCC(=O)NC8=O)C9=O)CC7)cc6)CC5)cn4c3=O)c2)(c2nncn2C)C1 ; "·HCOOH"]

### Synthesis of Compound 222.

The Compound 221-1 (500 mg) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 75% B to 75% B in 8 min; Wave Length: 220/254 nm; RT1 (min): 4.09; RT2 (min): 5.73; the second peak is product) to afford the crude product. The residue was purified by reverse flash chromatography with the following conditions (column, C18 silica gel; mobile phase, MeCN in water (0.1% FA), 0% to 40% gradient in 10 min; detector, UV 254 min) to afford Compound 222 (179.0 mg, 35.11%) as a yellow solid.

LC-MS-Compound 222: (ES, m/z): [M−HCOOH+H]⁺ 1031

H-NMR-Compound 222: (400 MHz, DMSO-d₆, δ ppm): 1.43-1.51 (m, 2H), 1.81-2.01 (m, 3H), 2.25-2.45 (m, 3H), 2.50-2.60 (m, 4H), 2.60-2.67 (m, 3H), 2.75-2.95 (m, 3H), 3.17 (s, 3H), 3.23 (s, 3H), 3.33-3.48 (m, 8H), 3.53-3.58 (m, 7H), 3.83-3.87 (m, 1H), 4.16-4.21 (m, 1H), 4.29-4.33 (m, 1H), 4.48 (s, 2H), 5.02-5.06 (d, 1H), 7.03-7.09 (m, 4H), 7.28-7.33 (m, 4H), 7.41-7.52 (m, 3H), 7.64-7.67 (m, 2H), 7.67-7.74 (m, 1H), 8.14 (s, 1H), 8.39 (s, 1H), 10.95 (s, 1H).

### Example 221. Compound 223

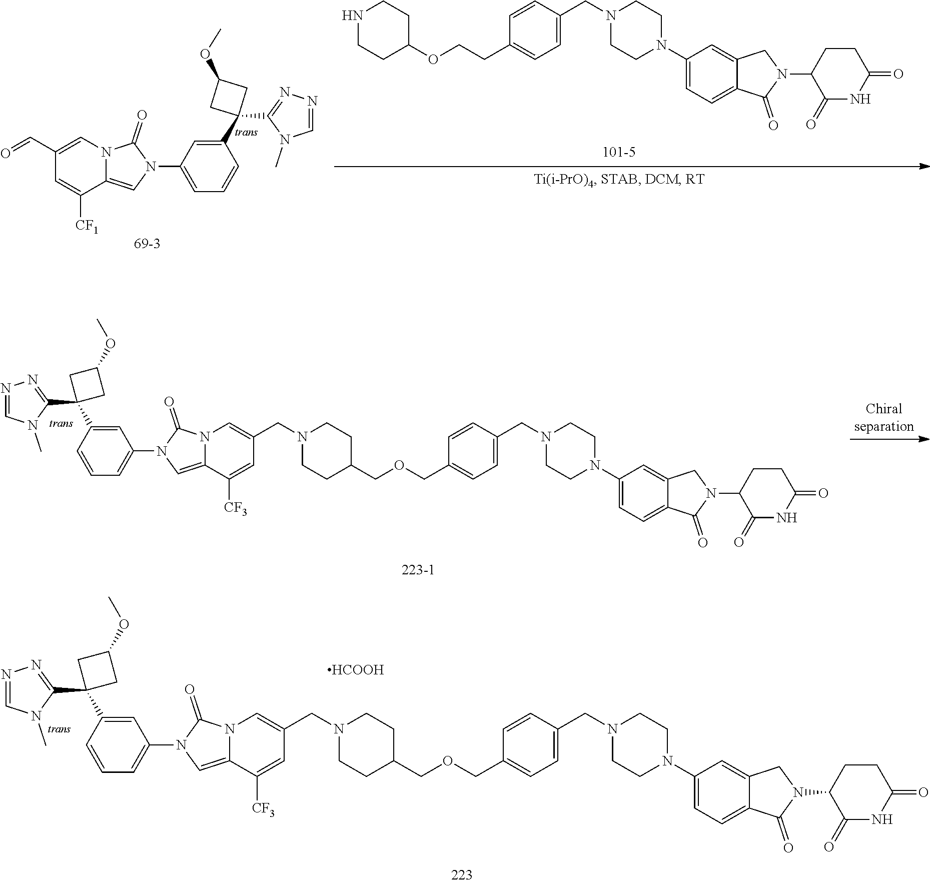

### Synthesis of Compound 223-1.

To a stirred solution of Compound 69-3 (1 g, 2.121 mmol, 1 equiv) and Compound 101-5 (1.16 g, 2.121 mmol, 1 equiv) in DCM (30 mL) was added Ti(Oi-Pr)₄ (1.81 g, 6.363 mmol, 3 equiv) at room temperature. The resulting mixture was stirred overnight at mom temperature. To the above mixture was added STAB (1.35 g, 6.363 mmol, 3 equiv) at room temperature. The resulting mixture was stirred for additional 6 h at room temperature. The reaction was quenched with 10% HOAc (aq.) at room temperature. The aqueous layer was extracted with EtOAc (80 mL). The aqueous layer was filtered, the filter cake was washed with water (10 mL). The filtrate was concentrated under reduced pressure. The residue was purified by reverse flash chromatography with the following conditions: column, C18 silica gel; mobile phase, MeCN in water (0.1% FA), 10% to 50% gradient in 20 min; detector, UV 254 nm. This resulted in Compound 223-1 (600 mg, 27.41%) as a yellow solid.

### Synthesis of Compound 223.

The Compound 223-1 (500 mg) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 90% B to 90% B in 8 min; Wave Length: 220/254 nm; RT1 (min): 4.41; RT2 (min): 5.54; the first peak is product) to afford the crude product. The residue was purified by reverse flash chromatography with the following conditions (column, C18 silica gel; mobile phase, MeCN in water (0.1% FA), 0% to 40% gradient in 10 min; detector, UV 254 nm) to afford Compound 223 (175.5 mg, 32.79%) as a yellow solid.

LC-MS-Compound 223: (ES, m/z): [M−HCOOH+H]⁺ 1001

H-NMR-Compound 223: (400 MHz, DMSO-d₆, δ ppm): 1.15-1.28 (m, 2H), 1.51-1.72 (m, 3H), 1.91-2.07 (m, 3H), 2.31-2.42 (m, 1H), 2.50-2.60 (m, 4H), 2.60-2.65 (m, 1H), 2.85-2.95 (m, 3H), 3.17 (s, 3H), 3.23 (s, 3H), 3.33-3.48 (m, 12H), 3.52 (s, 2H), 3.83-3.87 (m, 1H), 4.17-4.21 (m, 1H), 4.29-4.34 (m, 1H), 4.44 (s, 2H), 5.02-5.07 (d, 1H), 7.01-7.08 (m, 4H), 7.26-7.32 (m, 4H), 7.41-7.52 (m, 3H), 7.65-7.69 (m, 3H), 8.14 (s, 1H), 8.39 (s, 1H), 10.95 (s, 1H).

### Example 222. Compound 224

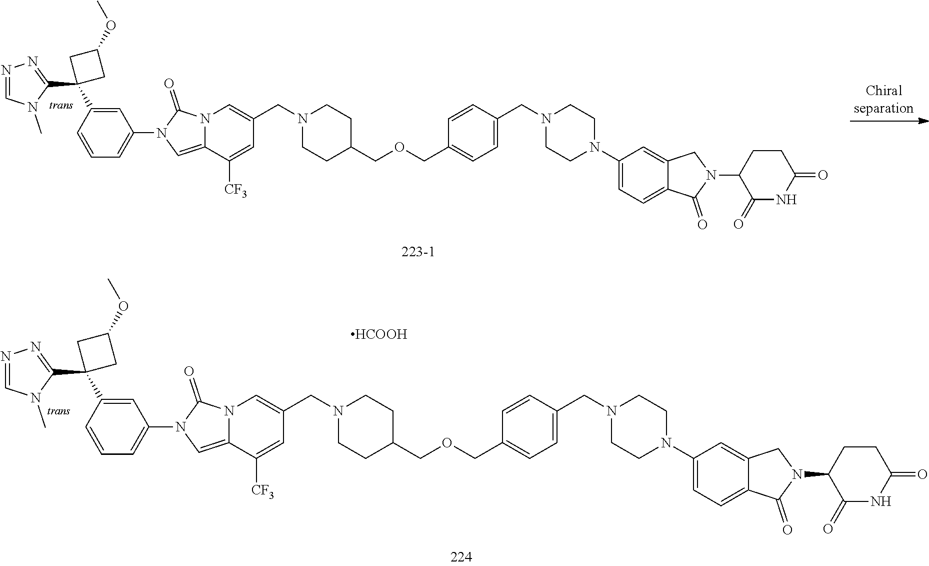 [SMILES: CO[C@H]1C[C@](c2cccc(-n3cc4c(C(F)(F)F)cc(CN5CCC(COCc6ccc(CN7CCN(c8ccc9c(c8)CN(C8CCC(=O)NC8=O)C9=O)CC7)cc6)CC5)cn4c3=O)c2)(c2nncn2C)C1 ; CO[C@H]1C[C@](c2cccc(-n3cc4c(C(F)(F)F)cc(CN5CCC(COCc6ccc(CN7CCN(c8ccc9c(c8)CN([C@H]8CCC(=O)NC8=O)C9=O)CC7)cc6)CC5)cn4c3=O)c2)(c2nncn2C)C1 ; "·HCOOH"]

### Synthesis of Compound 224.

The Compound 223-1 (500 mg) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 90% B to 90% B in 8 min; Wave Length: 220/254 nm; RT1 (min): 4.41; RT2 (min): 5.54; the second peak is product) to afford the crude product. The residue was purified by reverse flash chromatography with the following conditions (column, C18 silica gel; mobile phase, MeCN in water (0.1% FA), 0% to 40% gradient in 10 min; detector, UV 254 nm) to afford Compound 224 (173.6 mg, 32.60%) as a yellow solid.

LC-MS-Compound 224: (ES, m/z): [M−HCOOH+H]⁺ 1001

H-NMR-Compound 224: (400 MHz, DMSO-d₆, δ ppm): 1.15-1.28 (m, 2H), 1.51-1.72 (m, 3H), 1.91-2.07 (m, 3H), 2.31-2.42 (m, 1H), 2.50-2.60 (m, 4H), 2.60-2.65 (m, 1H), 2.85-2.95 (m, 3H), 3.17 (s, 3H), 3.23 (s, 3H), 3.33-3.48 (m, 12H), 3.52 (s, 2H), 3.83-3.87 (m, 1H), 4.17-4.21 (m, 1H), 4.29-4.34 (m, 1H), 4.44 (s, 2H), 5.02-5.07 (d, 1H), 7.01-7.08 (m, 4H), 7.26-7.32 (m, 4H), 7.41-7.52 (m, 3H), 7.65-7.69 (m, 3H), 8.14 (s, 1H), 8.39 (s, 1H), 10.95 (s, 1H).

### Example 223. Compound 225

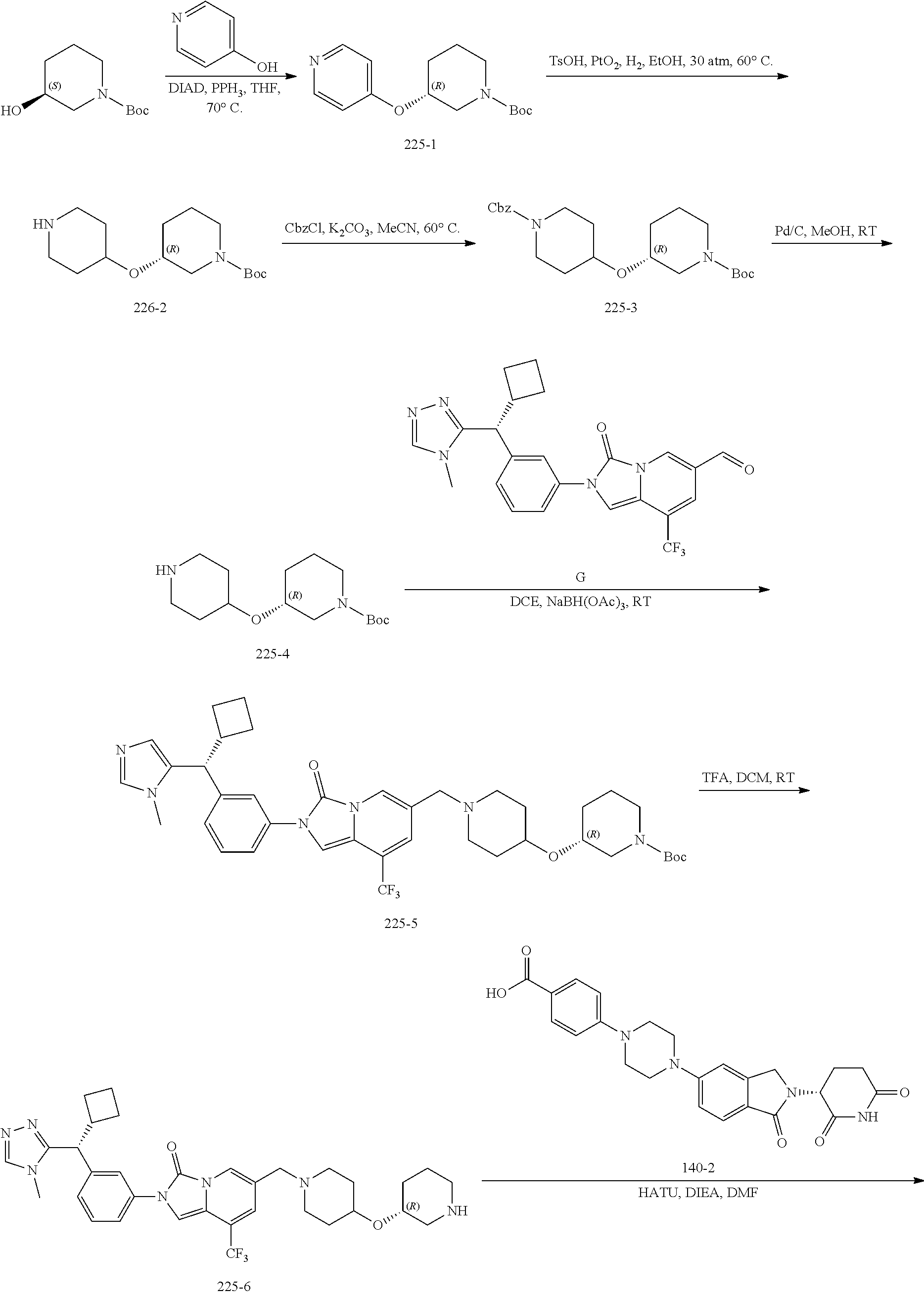

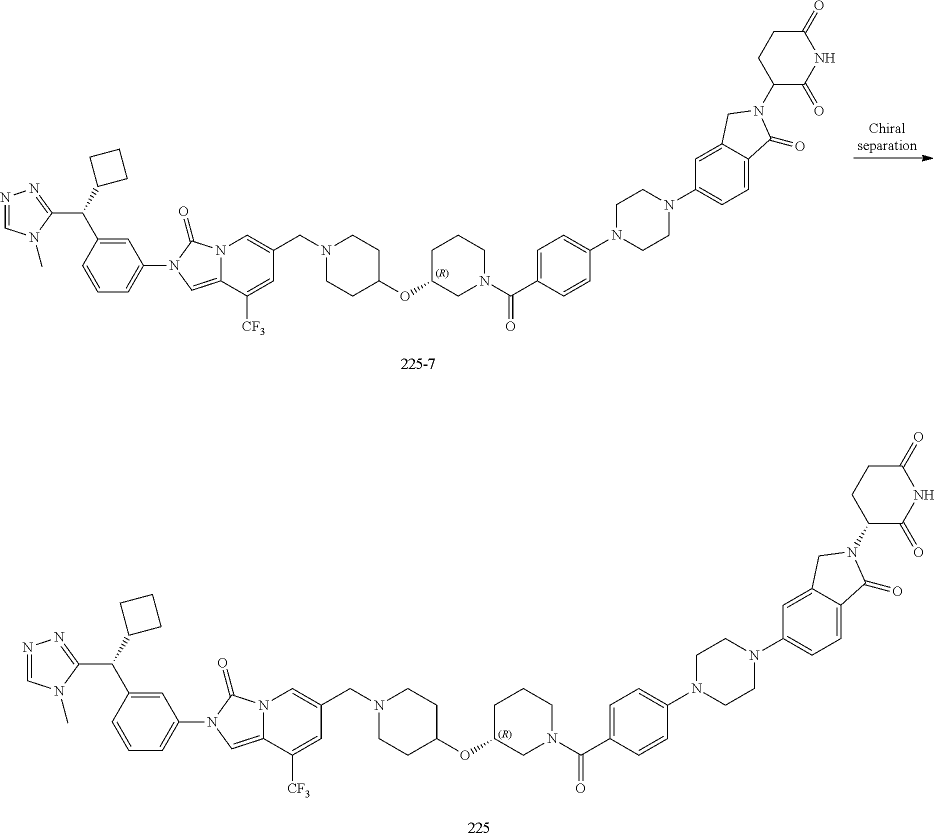

### Synthesis of Compound 225-1.

To a stirred solution of tert-butyl (3S)-3-hydroxypiperidine-1-carboxylate (50 g, 248.427 mmol, 1.5 equiv) and 4-hydroxypyridine (15.75 g, 165.618 mmol, 1 equiv) in THF (500 mL) were added DIAD (66.98 g, 331.236 mmol, 2 equiv) and PPH₃ (86.88 g, 331.236 mmol, 2 equiv) at room temperature under nitrogen atmosphere. The resulting mixture was stirred for overnight at 70° C. under nitrogen atmosphere. The reaction was quenched by the addition of Water (1000 mL) at room temperature. The aqueous layer was extracted with DCM (3×500 mL). The resulting mixture was concentrated under reduced pressure. The residue was purified by silica gel column chromatography, eluted with DCM/MeOH (30:1) to afford Compound 225-1 (6 g, 11.71%) as a white solid.

### Synthesis of Compound 225-2.

To a solution of Compound 225-1 (6 g, 21.555 mmol, 1 equiv) in 180 mL EtOH was added PtO₂ (0.98 g, 4.311 mmol, 0.2 equiv) and TsOH (0.74 g, 4.311 mmol, 0.2 equiv) in a pressure tank. The mixture was hydrogenated at 70° C. under 30 atm of hydrogen pressure for overnight, filtered through a Celite pad and concentrated under reduced pressure. This resulted in Compound 225-2 (5 g, 40.78%) as a brown solid.

### Synthesis of Compound 225-3.

To a stirred solution of Compound 225-2 (5 g, 8.790 mmol, 1 equiv, 50%) and K₂CO₃ (2.43 g, 17.580 mmol, 2 equiv) in MeCN (50 ml) were added CbzCl (3.00 g, 17.580 mmol, 2 equiv) at room temperature. The resulting mixture was stirred for overnight at 60° C. The reaction was quenched by the addition of Water (70 mL) at room temperature. The aqueous layer was extracted with DCM (3×50 mL). The resulting mixture was concentrated under reduced pressure. The residue was purified by silica gel column chromatography, eluted with PE/EA (5:1) to afford Compound 225-3 (2.1 g, 51.37%) as a brown solid.

### Synthesis of Compound 225-4.

To a solution of Compound 225-3 (2.1 g, 5.018 mmol, 1 equiv) in 30 mL MeOH was added Pd/C (10%, 197.57 mg) under nitrogen atmosphere. The mixture was hydrogenated at room temperature for overnight under hydrogen atmosphere using a hydrogen balloon, filtered through a Celite pad and concentrated under reduced pressure. This resulted in Compound 225-(1.6 g, 78.49%) as a brown solid.

### Synthesis of Compound 225-5.

To a stirred solution of Compound 225-4 (1.55 g, 5.450 mmol, 1 equiv) and Intermediate G (2.48 g, 5.450 mmol, 1 equiv) in DCE (20 ml) were added NaBH(OAc)₃ (2.31 g, 10.900 mmol, 2 equiv) at room temperature. The resulting mixture was stirred for overnight at room temperature. The reaction was quenched with Water (40 ml) at room temperature. The aqueous layer was extracted with DCM (3×15 mL). The resulting mixture was concentrated under reduced pressure. The residue was purified by silica gel column chromatography, eluted with DCM/MeOH (30:1) to afford Compound 225-5 (1.4 g, 30.16%) as a yellow solid.

### Synthesis of Compound 225-6.

To a stirred solution of Compound 225-5 (1.4 g, 1.934 mmol, 1 equiv) in DCM (15 mL) was added TFA (2.5 mL) at room temperature. The resulting mixture was stirred for 4 h at room temperature. The resulting mixture was concentrated under reduced pressure. The residue was purified by reversed-phase flash chromatography with the following conditions: column, C18 silica gel; mobile phase, MeCN in Water (0.1% FA), 30% to 45% gradient in 20 min; detector, UV 254 nm. This resulted in Compound 225-6 (1.1 g, 75.68%) as a yellow solid.

### Synthesis of Compound 225-7.

To a stirred solution of Compound 140-2 (0.75 g, 1.683 mmol, 1 equiv) and DIEA (0.54 g, 4.208 mmol, 2.5 equiv) in DMF (100 mL) was added HATU (1.60 g, 4.208 mmol, 2.5 equiv) at room temperature. The resulting mixture was stirred for 1 h at room temperature. To the above mixture was added Compound 225-6 (1.05 g, 1.683 mmol, 1 equiv) at room temperature. The resulting mixture was stirred for additional overnight at room temperature. The reaction was quenched with Water (100 mL) at room temperature. The aqueous layer was extracted with EtOAc (3×50 mL). The resulting mixture was concentrated under reduced pressure. The crude product (600 mg) was purified by Prep-HPLC with the following conditions (Column: Sunfire prep C18 column, 30*150 mm, 5 μm; Mobile Phase A: Water (0.1% FA), Mobile Phase B: ACN; Flow rate: 60 mL/min; Gradient: 22% B to 33% B in 7 min, 33% B: Wave Length: 254; 220 nm; RT1 (min): 6.98; to afford Compound 225-7 (250 mg, 13.66%) as a yellow solid.

### Synthesis of Compound 225-8.

The Compound 225-7 (300 mg, 0.285 mmol, 1 equiv) was purified by Chiral Separation with the following conditions (Column: CHIRALPAK IH, 2*25 cm, 5 μm, Mobile Phase A: MTBE (0.1% TEA)—HPLC, Mobile Phase B: DCM:ACN=1:1—HPLC; Flow rate: 20 mL/min; Gradient: 80% B to 80% B in 23 min; Wave Length: 220/254 nm; RT1 (min): 9.54; RT2 (min): 17.92; the first peak was product) to afford crude product (120 mg). The crude product (120 mg) was purified by reversed-phase flash chromatography with the following conditions: column, 40 g C18 silica gel; mobile phase, MeCN in Water (0.1% FA), 25% to 50% gradient in 10 min; detector, UV 254 nm to afford Compound 225 (76.4 mg, 24.55%) as a yellow solid.

LC-MS-Compound 225: (ES, m/z): [M+H]⁺ 1054

H-NMR-Compound 225: (400 MHz, DMSO-d6, δ ppm): 1.24-1.59 (m, 4H), 1.67-1.79 (m, 8H), 1.94-2.09 (m, 1H), 2.11-2.22 (m, 3H), 2.35-2.49 (m, 1H), 2.50-2.67 (m, 3H), 2.90-2.95 (m, 2H), 3.20-3.25 (m, 3H), 3.34-3.39 (m, 6H), 3.40-3.42 (m, 4H), 3.44-3.45 (m, 7H), 4.19-4.26 (m, 2H), 4.32-4.36 (d, 1H), 5.03-5.08 (m, 1H), 7.00-7.02 (m, 3H), 7.11-7.13 (m, 2H), 7.18-7.20 (m, 1H), 7.30-7.35 (m, 3H), 7.41-7.41 (m, 1H), 7.43-7.45 (m, 1H), 7.53-7.67 (m, 1H), 7.68-7.73 (s, 1H), 8.14 (s, 1H), 8.32 (s, 1H), 10.96 (s, 1H).

### Example 224. Compound 226

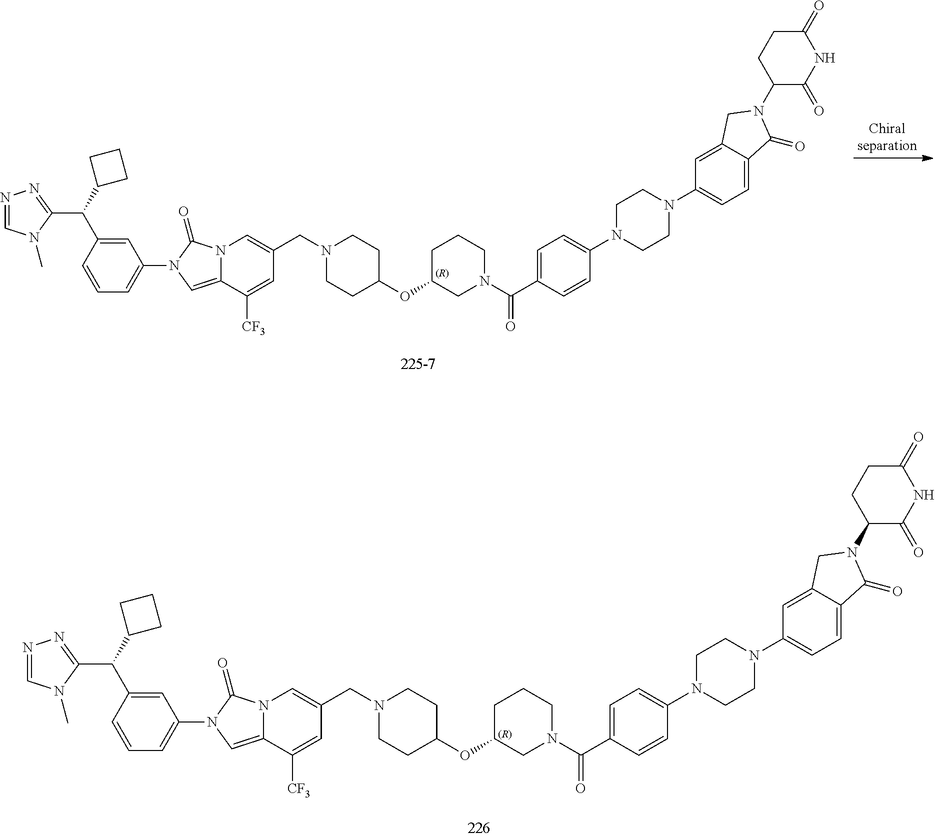

### Synthesis of Compound 226.

The Compound 225-7 (300 mg, 0.285 mmol, 1 equiv) was purified by Chiral Separation with the following conditions (Column: CHIRALPAK IH, 2*25 cm, 5 μm, Mobile Phase A: MTBE (0.1% TEA)—HPLC, Mobile Phase B: DCM:ACN=1:1—HPLC; Flow rate: 20 mL/min; Gradient: 80% B to 80% B in 23 min; Wave Length: 220/254 nm; RT1 (min): 9.54; RT2 (min): 17.92; the second peak was product) to afford crude product (140 mg). The crude product (140 mg) was purified by reversed-phase flash chromatography with the following conditions: column, 40 g C18 silica gel; mobile phase, MeCN in Water (0.1% FA), 25% to 50% gradient in 10 min; detector, UV 254 nm to afford Compound 226 (109.7 mg, 34.96%) as a yellow solid.

LC-MS-Compound 226: (ES, m/z): [M+H]⁺ 1054

H-NMR-Compound 226: (400 MHz, DMSO-d6, δ ppm): 1.28-1.67 (m, 4H), 1.69-1.79 (m, 8H), 1.95-1.97 (m, 1H), 2.07-2.09 (m, 3H), 2.33-2.49 (m, 1H), 2.50-2.67 (m, 3H), 2.90-2.95 (m, 2H), 3.25-3.34 (m, 3H), 3.38-3.42 (m, 7H), 3.44-3.45 (m, 4H), 3.46-4.47 (m, 6H), 4.19-4.26 (m, 2H), 4.31-4.35 (d, 1H), 5.03-5.08 (d, 1H), 7.00-7.02 (m, 3H), 7.11-7.13 (m, 3H), 7.18-7.30 (m, 3H), 7.41-7.45 (m, 1H), 7.53-7.67 (m, 1H), 7.68-7.73 (m, 1H), 8.32 (s, 1H), 10.96 (s, 1H).

### Example 225. Compound 227

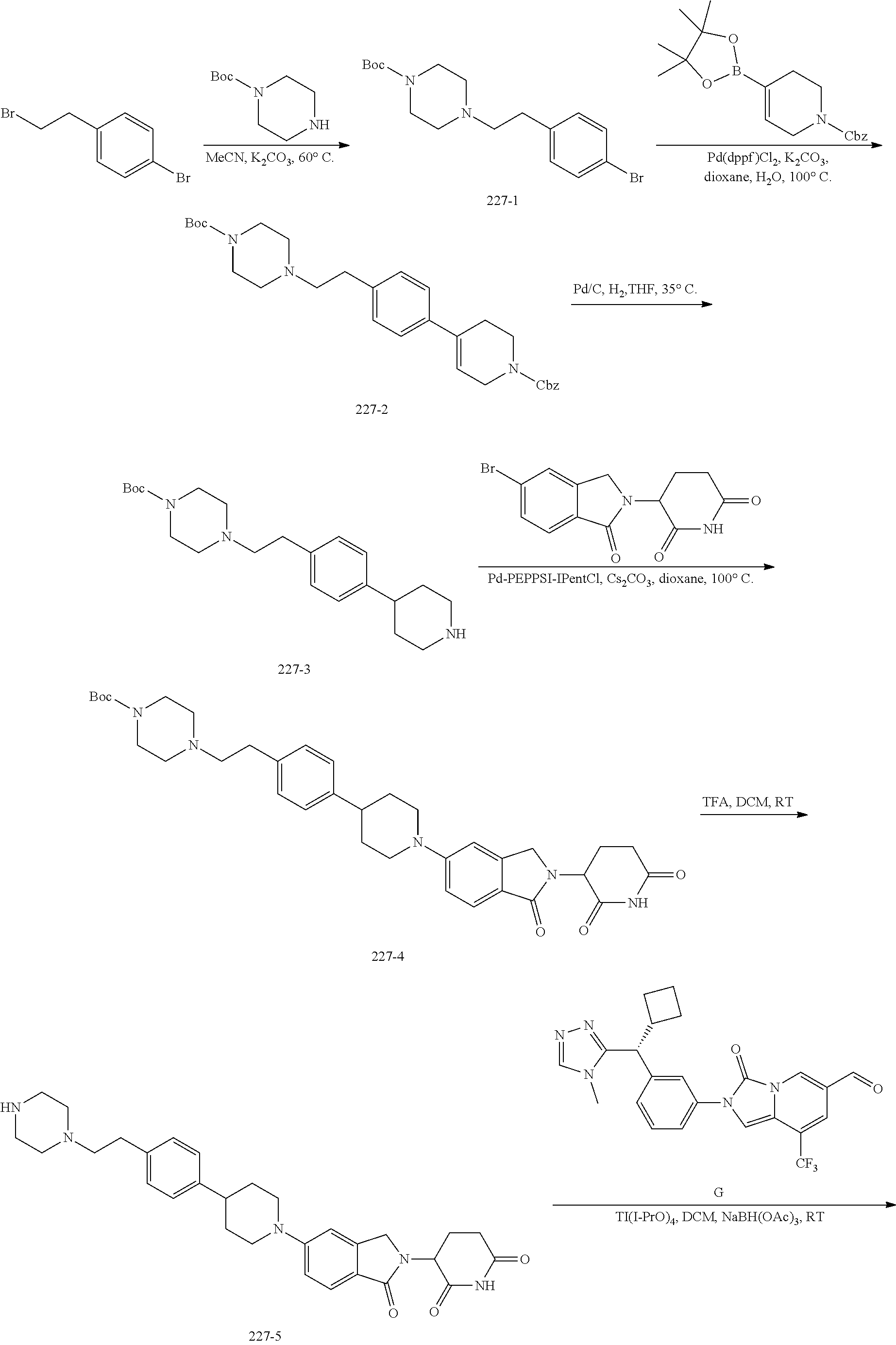

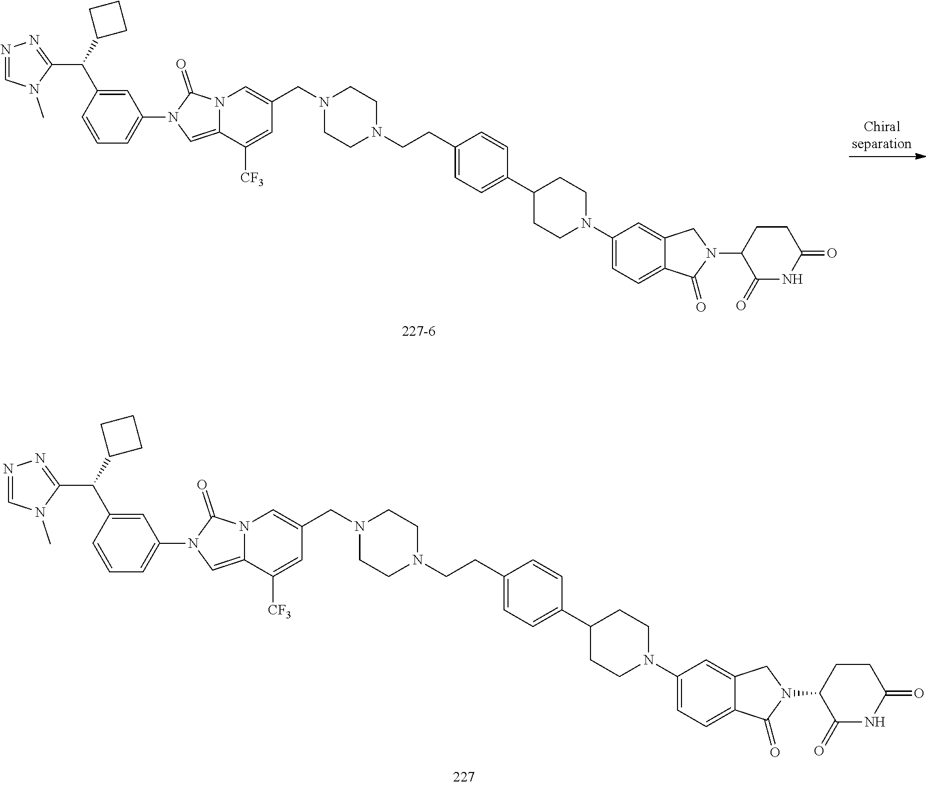 [SMILES: Cn1cnnc1[C@@H](c1cccc(-n2cc3c(C(F)(F)F)cc(CN4CCN(CCc5ccc(C6CCN(c7ccc8c(c7)CN(C7CCC(=O)NC7=O)C8=O)CC6)cc5)CC4)cn3c2=O)c1)C1CCC1 ; Cn1cnnc1[C@@H](c1cccc(-n2cc3c(C(F)(F)F)cc(CN4CCN(CCc5ccc(C6CCN(c7ccc8c(c7)CN([C@@H]7CCC(=O)NC7=O)C8=O)CC6)cc5)CC4)cn3c2=O)c1)C1CCC1]

### Synthesis of Compound 227-1.

To a stirred solution of 1-bromo-4-(2-bromoethyl)benzene (15.0 g, 56.83 mmol, 1.0 equiv) and tert-butyl piperazine-1-carboxylate (10.6 g, 56.83 mmol, 1.0 equiv) in MeCN (150 mL) was added K₂CO₃ (11.8 g, 85.24 mmol, 1.5 equiv) at room temperature. The resulting mixture was stirred for overnight at 60° C. The reaction was quenched with water at room temperature. The aqueous layer was extracted with CH₂Cl₂ (3×200 mL). The residue was purified by silica gel column chromatography, eluted with PE/EA (10:1) to afford Compound 227-1 (14.0 g, 60%) as a white oil.

### Synthesis of Compound 227-2.

To a stirred solution of Compound 227-1 (19.5 g, 56.86 mmol, 1.5 equiv) and tert-butyl 4-[2-(4-bromophenyl)ethyl]piperazine-1-carboxylate (14.0 g, 37.91 mmol, 1.0 equiv) in 1,4-dioxane (1.2 mL) were added H₂O (28 mL) and K₂CO₃ (15.7 g, 113.73 mmol, 3.0 equiv) at room temperature. The resulting mixture was stirred for overnight at 100° C. under nitrogen atmosphere. The reaction was quenched by the addition of sat. NH₄Cl (aq.) (300 mL) at room temperature. The resulting mixture was extracted with EtOAc (3×100 mL), dried over anhydrous Na₂SO₄. After filtration, the filtrate was concentrated under reduced pressure. The residue was purified by silica gel column chromatography, eluted with PE/EA (8:1) to afford Compound 227-2 (14.0 g, 69%) as a yellow solid.

### Synthesis of Compound 227-3.

To a solution of Compound 227-2 (5.0 g, 9.89 mmol, 1.0 equiv) in 100 mL THF was added Pd/C (10%, 510 mg) under nitrogen atmosphere in a 500 mL round bottom flask. The mixture was hydrogenated at 50° C. for 2 h under hydrogen atmosphere using a hydrogen balloon, filtered through a Celite pad and concentrated under reduced pressure. This resulted in Compound 227-3 (3.0 g, 81%) as a yellow oil.

### Synthesis of Compound 227-4.

Into a 100 mL 3-necked round bottom flask were added Compound 227-3 (3.0 g, 8.03 mmol, 1.0 equiv), dioxane (30 mL), 3-(5-bromo-1-oxo-3H-isoindol-2-yl)piperidine-2,6-dione (3.1 g, 9.64 mmol, 1.2 equiv), Cs₂CO₃ (7.8 g, 24.09 mmol, 3.0 equiv) and {1,3-bis[2,6-bis(pentan-3-yl)phenyl]-4,5-dichloro-2,3-dihydro-1H-imidazol-2-yl}dichloro(2-methyl-1lambda4-pyridin-1-yl)palladium (200 mg, 0.24 mmol, 0.03 equiv) at room temperature. The resulting mixture was stirred for overnight at 100° C. under N₂ atmosphere. The reaction was quenched by the addition of 10% CH₃COOH in H₂O (200 ml) at room temperature. The aqueous layer was extracted with DCM/MeOH=10:1 (3×100 mL). The resulting mixture was concentrated under reduced pressure. The residue was purified by silica gel column chromatography, eluted with DCM/MeOH (30:1) to afford Compound 227-4 (1.5 g, 30%) as a yellow solid.

### Synthesis of Compound 227-5.

To a stirred solution of Compound 227-4 (1.0 g, 1.62 mmol, 1.0 equiv) in DCM (9 mL) was added TFA (3 mL) at room temperature under nitrogen atmosphere. The resulting mixture was stirred for 6 h at room temperature under nitrogen atmosphere. The resulting mixture was diluted with CH₂Cl₂ (50 mL). The resulting mixture was concentrated under reduced pressure. The crude product was used in the next step directly without further purification. This resulted in Compound 227-5 (800 mg, 90%) as a brown oil.

### Synthesis of Compound 227-6.

To a stirred solution of Compound 227-5 (400 mg, 0.77 mmol, 1.0 equiv) and Intermediate G (423 mg, 0.93 mmol, 1.2 equiv) in DCM (4 mL) were added Ti(Oi-Pr)₄ (882 mg, 3.10 mmol, 4.0 equiv) at room temperature under nitrogen atmosphere. The resulting mixture was stirred for overnight at room temperature under nitrogen atmosphere. To the above mixture was added NaBH(OAc)₃ (493 mg, 2.33 mmol, 3.0 equiv) at room temperature. The resulting mixture was stirred for additional 4 h at room temperature. The reaction was quenched by the addition of water/HOAc=10:1 (50 mL) at room temperature. The aqueous layer was extracted with CH₂Cl₂/MeOH=5:1 (3×30 mL). The resulting mixture was concentrated under reduced pressure. The residue was purified by reversed-phase flash chromatography with the following conditions: column, C18 silica gel; mobile phase, MeCN in water (0.1% FA), 10% to 50% gradient in 30 min; detector, UV 254 nm. The crude product (200 mg) was purified by Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 25% B to 38% B in 8 min, 38% B: Wave Length: 254; 220 nm; RT1 (min): 7.8; Number of Runs: 0) to afford Compound 227-6 (80 mg, 10%) as a yellow solid.

### Synthesis of Compound 227.

The Compound 227-6 (80 mg, 0.08 mmol, 1.0 equiv) was purified by Prep-Chiral-HPLC with the following conditions (Column: CHIRALPAK IH, 2*25 cm, 5 μm, Mobile Phase A; MTBE (0.1% TEA), Mobile Phase B: DCM:ACN=1:1; Flow rate: 20 mL/min; Gradient: 80% B to 80% B in 10 min; Wave Length: 220/254 nm; RT1 (min): 5.12; RT2 (min): 7.04; the first peak was product) to afford crude product. The crude product (25 mg) was purified by Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 25% B to 38% B in 8 min, 38% B: Wave Length: 254; 220 nm; RT1 (min): 7.8; Number Of Runs: 0) to afford Compound 227 (11 mg, 14%) as a yellow solid.

LCMS-Compound 228: (ES, m/z): [M+H]⁺ 478

NMR-Compound 228: (400 MHz, DMSO-d6, δ ppm): 1.69-1.86 (m, 10H), 1.87-1.99 (m, 1H), 2.01-2.09 (m, 1H), 2.38-2.39 (m, 4H), 2.49-2.67 (m, 5H), 2.70-2.73 (m, 4H), 2.89-2.95 (m, 4H), 3.32-3.35 (m, 3H), 3.43 (s, 3H), 4.02-4.19 (m, 2H), 4.23-4.36 (m, 3H), 5.04-5.09 (m, 1H), 7.01 (s, 1H), 7.08-7.11 (t, 2H), 7.14-7.21 (m, 5H), 7.32 (s, 1H), 7.42-7.46 (m, 1H), 7.51-7.73 (m, 4H), 8.32 (s, 1H), 10.95 (s, 1H).

### Example 226. Compound 228

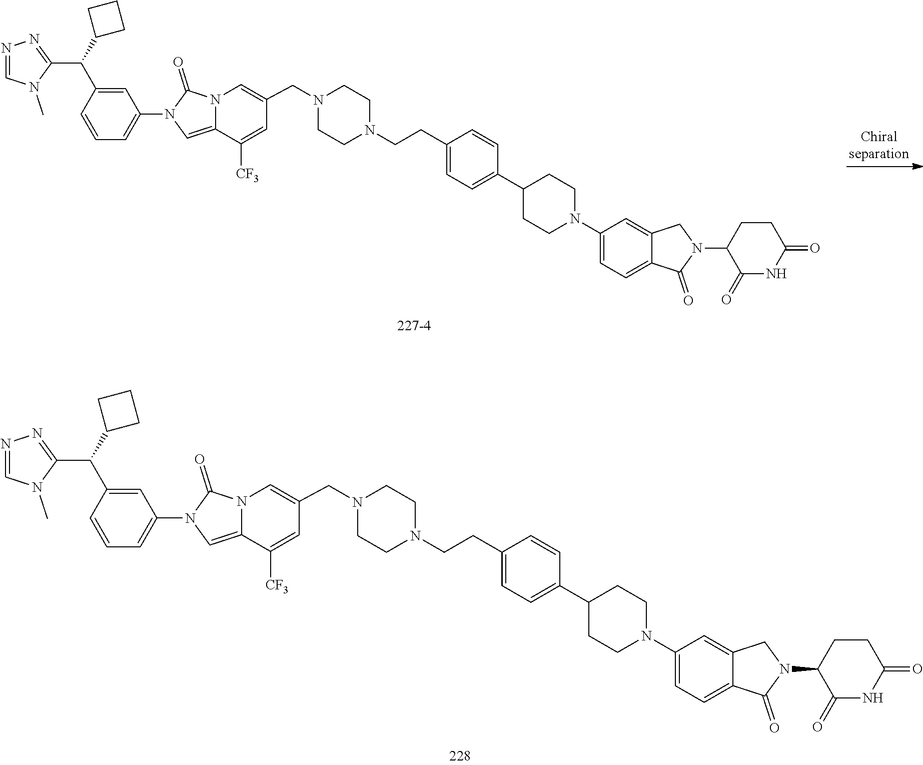 [SMILES: Cn1cnnc1[C@@H](c1cccc(-n2cc3c(C(F)(F)F)cc(CN4CCN(CCc5ccc(C6CCN(c7ccc8c(c7)CN(C7CCC(=O)NC7=O)C8=O)CC6)cc5)CC4)cn3c2=O)c1)C1CCC1 ; Cn1cnnc1[C@@H](c1cccc(-n2cc3c(C(F)(F)F)cc(CN4CCN(CCc5ccc(C6CCN(c7ccc8c(c7)CN([C@H]7CCC(=O)NC7=O)C8=O)CC6)cc5)CC4)cn3c2=O)c1)C1CCC1]

### Synthesis of Compound 228.

The Compound 227-4 (80 mg, 0.08 mmol, 1.0 equiv) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 80% B to 80% B in 10 min; Wave Length: 220/254 nm; RT1 (min): 5.12; RT2 (min): 7.04; the second peak was product) to afford crude product. The residue was purified by reversed-phase flash chromatography with the following conditions: column, C18 silica gel; mobile phase, MeCN in water (0.1% FA), 10% to 50% gradient in 20 min; detector, UV 254 nm. This resulted in Compound 227 (16 mg, 21%) as a yellow solid.

LCMS-Compound 228: (ES, m/z): [M+H]⁺ 955

NMR-Compound 228: (400 MHz, DMSO-d6, δ ppm): 1.69-2.13 (m, 12H), 2.36-2.44 (m, 7H), 2.49-2.50 (m, 2H), 2.56-2.61 (m, 3H), 2.67-2.69 (m, 3H), 2.71-2.89 (m, 2H), 2.92-2.95 (m, 3H), 3.43 (s, 3H), 3.99-4.02 (m, 2H), 4.19-4.36 (m, 3H), 5.04-5.06 (m, 1H), 7.01 (s, 1H), 7.08-7.20 (m, 7H), 7.31 (s, 1H), 7.42-7.46 (t, 1H), 7.51-7.53 (d, 1H), 7.67-7.73 (m, 3H), 8.32 (s, 1H), 10.95 (s, 1H).

### Example 227. Compound 229

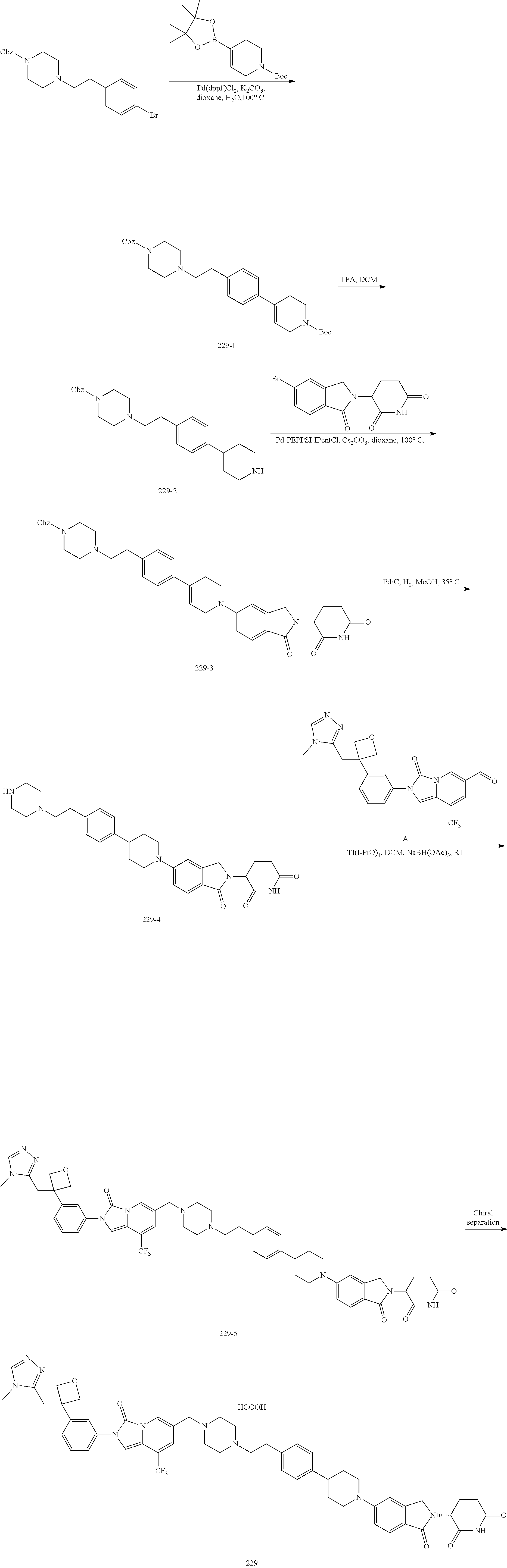

### Synthesis of Compound 229-1.

Into a 250 mL 3-necked round bottom flask were added benzyl 4-[2-(4-bromophenyl) ethyl] piperazine-1-carboxylate (10 g, 24.794 mmol, 1 equiv), dioxane (80 mL), H₂O (20 mL), tert-butyl 4-(4,4,5,5-tetramethyl-1,3,2-dioxaborolan-2-yl)-3,6-dihydro-2H-pyridine-1-carboxylate (11.50 g, 37.191 mmol, 1.5 equiv), K₂CO₃ (10.28 g, 74.382 mmol, 3 equiv) and Pd(dppf)Cl₂ (0.36 g, 0.496 mmol, 0.02 equiv) at room temperature. The resulting mixture was stirred for overnight at 100° C. under nitrogen. The reaction was quenched by the addition of sat. NH₄Cl (100 mL) at room temperature. The aqueous layer was extracted with EA (3×100 mL). The resulting mixture was concentrated under reduced pressure. The residue was purified by silica gel column chromatography, eluted with PE/EA (10:1) to afford Compound 229-1 (12 g, 95.71%) as a yellow oil.

### Synthesis of Compound 229-2.

Into a 250 mL round bottom flask were added Compound 229-1 (12 g, 23.731 mmol, 1 equiv), DCM (100 mL) and TFA (20 mL) at room temperature. The resulting mixture was stirred for 2 h at room temperature. The resulting mixture was concentrated under reduced pressure. The residue was purified by silica gel column chromatography, eluted with DCM/MeOH (10:1) to afford Compound 229-2 (9 g, 93.52%) as a yellow oil.

### Synthesis of Compound 229-3.

Into a 250 mL round bottom flask were added Compound 229-2 (4.5 g, 11.096 mmol, 1 equiv), dioxane (50 mL), 3-(5-bromo-1-oxo-3H-isoindol-2-yl) piperidine-2,6-dione (4.30 g, 13.315 mmol, 1.2 equiv), Cs₂CO₃ (10.85 g, 33.288 mmol, 3 equiv) and Pd-PEPPSI-IPentCl (0.28 g, 0.333 mmol, 0.03 equiv) at room temperature. The resulting mixture was stirred for overnight at 100° C. under nitrogen. The reaction was quenched by the addition of 10% AcOH in water (200 mL) at room temperature. The aqueous layer was extracted with DCM/MeOH=10:1 (3×100 mL). The resulting mixture was concentrated under reduced pressure. The residue was purified by silica gel column chromatography, eluted with DCM/MeOH (30:1) to afford Compound 229-3 (2 g, 27.82%) as a yellow solid.

### Synthesis of Compound 229-4.

To a solution of Compound 229-3 (2 g, 3.087 mmol, 1 equiv) in 100 mL MeOH was added Pd/C (10%, 0.2 g) under nitrogen atmosphere in a 250 mL round bottom flask. The mixture was hydrogenated at room temperature for 2 h under hydrogen atmosphere using a hydrogen balloon, filtered through a Celite pad and concentrated under reduced pressure. This resulted in Compound 229-4 (1 g, 62.81%) as a light yellow solid.

### Synthesis of Compound 229-5.

Into a 30 mL sealed tube were added Compound 229-4 (1 g, 1.939 mmol, 1 equiv), DCM (10 mL), Intermediate A (0.98 g, 2.133 mmol, 1.1 equiv) and Ti(Oi-Pr)₄ (2.20 g, 7.756 mmol, 4 equiv) at room temperature. The resulting mixture was stirred for overnight at room temperature. To the above mixture was added NaBH(OAc)₃ (1.23 g, 5.817 mmol, 3 equiv) at mom temperature. The resulting mixture was stirred for additional 2 h at room temperature. The reaction was quenched by the addition of 10% AcOH in water (100 mL) at room temperature. The aqueous layer was extracted with DCM/MeOH=10:1 (3×50 mL). The resulting mixture was concentrated under reduced pressure. The crude product was purified by Prep-HPLC with the following conditions (Column: Sunfire prep C18 column, 30*150 mm, 5 μm; Mobile Phase A: Water (0.1% FA), Mobile Phase B: ACN; Flow rate: 60 mL/min; Gradient: 40% B to 55% B in 10 min, 55% B; Wave Length: 254; 220 nm; RT1 (min): 9.25) to afford Compound 229-5 (150 mg, 8.08%) as a yellow solid.

### Synthesis of Compound 229.

The Compound 229-5 (150 mg) was purified by Prep-Chiral-HPLC with the following conditions (Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 80% B to 80% B in 14 min; Wave Length: 220/254 nm; RT1 (min): 7.10; RT2 (min): 10.61; The first peak was the product. Sample Solvent: DCM:ACN=1:1—HPLC; Injection Volume: 0.85 mL; Number of Runs: 4) to afford crude product. The crude product was purified by Prep-HPLC with the following conditions (Column: Sunfire prep C18 column, 30*150 mm, 5 μm; Mobile Phase A: Water (0.1% FA), Mobile Phase B: ACN; Flow rate: 60 mL/min; Gradient: 20% B to 32% B in 8 min, 32% B; Wave Length: 254; 220 nm; RT1 (min): 7.87) to afford Compound 229 (22.5 mg, 14.92%) as a yellow solid.

LC-MS-Compound 229: (ES, m/z): [M−HCOOH+H]⁺ 957

H-NMR-Compound 229: 1H NMR (300 MHz, DMSO-d₆) δ 1.68-1.72 (m, 2H), 1.82-1.86 (m, 2H), 1.92-1.96 (m, 1H), 2.32-2.45 (m, 8H), 2.50-2.70 (m, 5H), 2.81-2.89 (m, 3H), 2.90-2.96 (m, 4H), 3.29-3.33 (m, 3H), 3.51-3.53 (m, 2H), 3.99-4.02 (m, 2H), 4.18-4.23 (m, 1H), 4.31-4.35 (m, 1H), 4.90-4.95 (m, 4H), 5.03-5.07 (m, 1H), 6.87-6.89 (m, 1H), 7.01-7.03 (m, 1H), 7.07-7.09 (m, 2H), 7.11-7.18 (m, 4H), 7.32-7.35 (m, 1H), 7.37-7.42 (m, 2H), 7.51-7.53 (m, 1H), 7.67-7.70 (m, 1H), 7.74-7.76 (m, 1H), 8.18-8.20 (m, 1H), 10.98 (s, 1H).

### Example 228. Compound 230

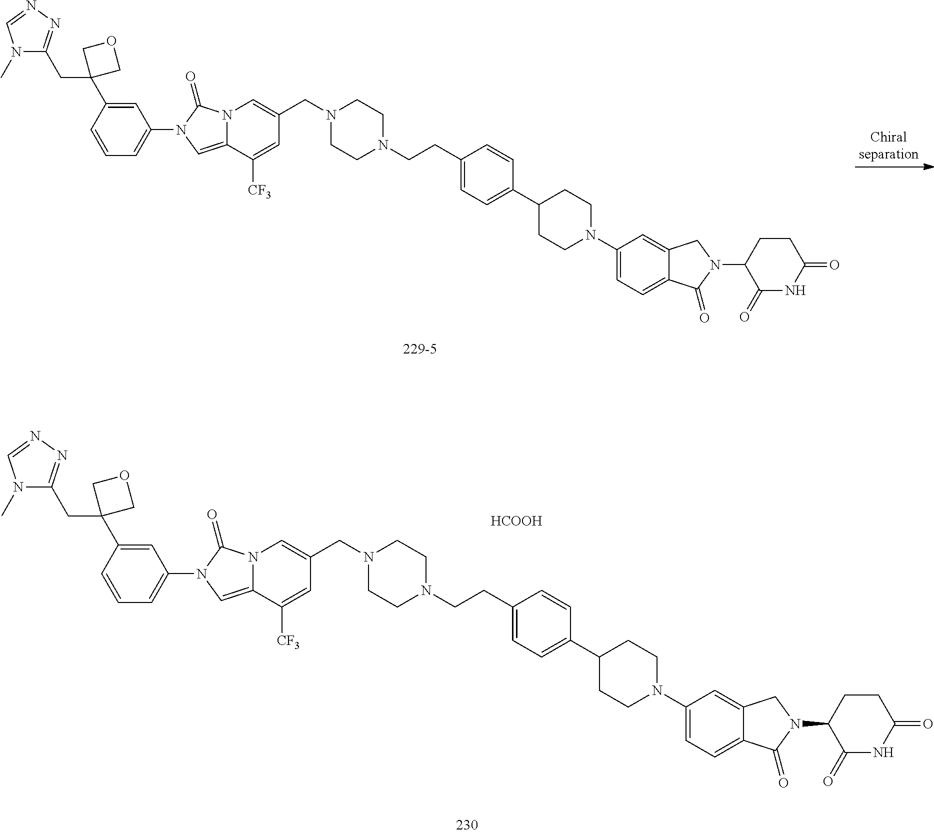 [SMILES: Cn1cnnc1CC1(c2cccc(-n3cc4c(C(F)(F)F)cc(CN5CCN(CCc6ccc(C7CCN(c8ccc9c(c8)CN(C8CCC(=O)NC8=O)C9=O)CC7)cc6)CC5)cn4c3=O)c2)COC1 ; Cn1cnnc1CC1(c2cccc(-n3cc4c(C(F)(F)F)cc(CN5CCN(CCc6ccc(C7CCN(c8ccc9c(c8)CN([C@H]8CCC(=O)NC8=O)C9=O)CC7)cc6)CC5)cn4c3=O)c2)COC1 ; "HCOOH"]

### Synthesis of Compound 230.

The Compound 229-5 (150 mg) was purified by Prep-Chiral-HPLC with the following conditions (Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 80% B to 80% B in 14 min; Wave Length: 220/254 nm; RT1 (min): 7.10; RT2 (min): 10.61; The second peak was the product. Sample Solvent: DCM:ACN=1:1—HPLC; Injection Volume: 0.85 mL; Number of Runs: 4) to afford crude product. The crude product was purified by Prep-HPLC with the following conditions (Column: Sunfire prep C18 column, 30*150 mm, 5 μm; Mobile Phase A: Water (0.1% FA), Mobile Phase B: ACN; Flow rate: 60 mL/min; Gradient: 20% B to 32% B in 8 min, 32% B; Wave Length: 254; 220 nm; RT1 (min): 7.87) to afford Compound 230 (34.8 mg, 22.30%) as a yellow solid.

LC-MS-Compound 230: (ES, m/z): [M−HCOOH+H]⁺ 957

H-NMR-Compound 230: 1H NMR (300 MHz, DMSO-d₆) δ 1.69-1.72 (m, 2H), 1.83-1.86 (m, 2H), 1.91-1.93 (m, 1H), 2.32-2.39 (m, 9H), 2.49-2.51 (m, 1H), 2.66-2.70 (m, 4H), 2.89-2.96 (m, 6H), 3.29-3.35 (m, 3H), 3.51-3.53 (m, 2H), 3.99-4.02 (m, 2H), 4.19-4.23 (m, 1H), 4.31-4.36 (m, 1H), 4.91-4.96 (m, 4H), 5.03-5.06 (m, 1H), 6.87-6.89 (m, 1H), 7.01-7.03 (m, 1H), 7.00-7.15 (m, 6H), 7.32-7.35 (m, 1H), 7.37-7.42 (m, 2H), 7.51-7.53 (m, 1H), 7.67-7.70 (m, 1H), 7.74-7.76 (m, 1H), 8.18-8.20 (m, 1H), 10.98 (s, 1H).

### Example 229. Compound 231

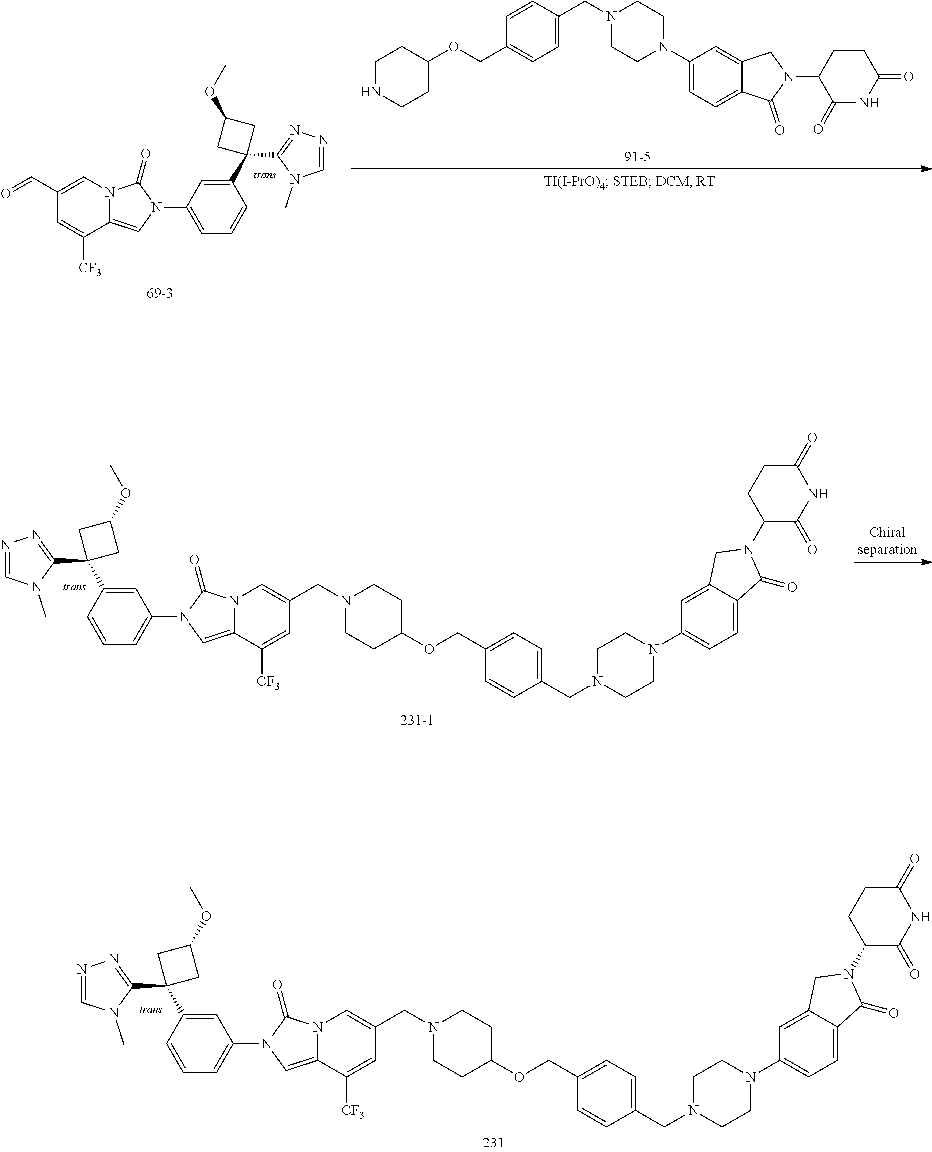 [SMILES: CO[C@H]1C[C@](c2cccc(-n3cc4c(C(F)(F)F)cc(C=O)cn4c3=O)c2)(c2nncn2C)C1 ; O=C1CCC(N2Cc3cc(N4CCN(Cc5ccc(COC6CCNCC6)cc5)CC4)ccc3C2=O)C(=O)N1 ; "91-5" ; CO[C@H]1C[C@](c2cccc(-n3cc4c(C(F)(F)F)cc(CN5CCC(OCc6ccc(CN7CCN(c8ccc9c(c8)CN(C8CCC(=O)NC8=O)C9=O)CC7)cc6)CC5)cn4c3=O)c2)(c2nncn2C)C1 ; CO[C@H]1C[C@](c2cccc(-n3cc4c(C(F)(F)F)cc(CN5CCC(OCc6ccc(CN7CCN(c8ccc9c(c8)CN([C@@H]8CCC(=O)NC8=O)C9=O)CC7)cc6)CC5)cn4c3=O)c2)(c2nncn2C)C1]

### Synthesis of Compound 231-1.

A solution of Compound 69-3 (709.39 mg, 1.505 mmol, 1 equiv) and Compound 91-5 (800 mg, 1.505 mmol, 1.00 equiv) in DCM (24 mL) was treated with Ti(Oi-Pr)₄ (1710.69 mg, 6.020 mmol, 4 equiv) for 1 h at room temperature under nitrogen atmosphere followed by the addition of STAB (956.74 mg, 4.515 mmol, 3 equiv) at room temperature. The resulting mixture was stirred for 4 h at room temperature under nitrogen atmosphere. The reaction was quenched by the addition of Water/HOAc=10/1 (100 mL) at room temperature. The resulting mixture was extracted with CH2Cl₂/MeOH=6/1 (3×150 mL). The combined organic layers were concentrated under reduced pressure. The residue was purified by reversed-phase flash chromatography with the following conditions: column, C18 silica gel; mobile phase, MeCN in Water (0.1% FA), 10% to 45% gradient in 30 min; detector, UV 254 nm. This resulted in Compound 231-1 (609 mg, 41.00%) as a yellow solid.

### Synthesis of Compound 231.

Compound 231-1 (400 mg, 0.405 mmol, 1 equiv) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 60% B to 60% B in 15 min; Wave Length: 220/254 nm; RT1 (min): 5.82; RT2 (min): 9.82; The first peak was product) to afford Compound 231 (155.9 mg, 38.98%) as a yellow solid.

LCMS-Compound 231: (ES, m/z): [M+H]⁺ 987

NMR-Compound 231: (400 MHz, DMSO-d6, δ ppm): 1.52-1.54 (m, 2H), 1.86-1.96 (m, 3H), 2.08-2.18 (m, 2H), 2.34-2.40 (m, 2H), 2.51-2.54 (m, 1H), 2.60-2.69 (m, 2H), 2.82-2.89 (m, 1H), 3.16-3.23 (m, 4H), 3.24-3.29 (m, 4H), 3.30-3.41 (m, 12H), 3.53 (s, 2H), 3.84-3.87 (m, 1H), 4.17-4.21 (d, 1H), 4.30-4.35 (d, 1H), 4.49 (s, 2H), 5.04-5.07 (m, 1H), 7.02-7.09 (m, 4H), 7.26-7.31 (m, 4H), 7.41-7.53 (m, 3H), 7.65-7.69 (m, 2H), 8.15 (s, 1H), 8.39 (s, 1H), 10.99 (s, 1H).

### Example 230. Compound 232

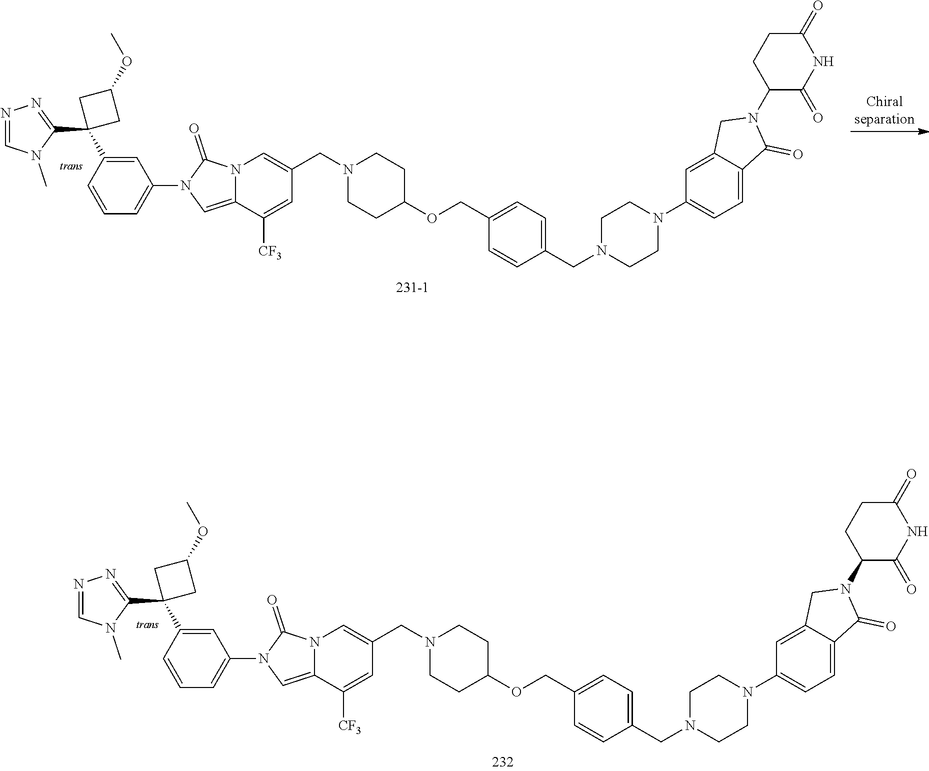 [SMILES: CO[C@H]1C[C@](c2cccc(-n3cc4c(C(F)(F)F)cc(CN5CCC(OCc6ccc(CN7CCN(c8ccc9c(c8)CN(C8CCC(=O)NC8=O)C9=O)CC7)cc6)CC5)cn4c3=O)c2)(c2nncn2C)C1 ; CO[C@H]1C[C@](c2cccc(-n3cc4c(C(F)(F)F)cc(CN5CCC(OCc6ccc(CN7CCN(c8ccc9c(c8)CN([C@H]8CCC(=O)NC8=O)C9=O)CC7)cc6)CC5)cn4c3=O)c2)(c2nncn2C)C1]

### Synthesis of Compound 232.

Compound 231-1 (400 mg, 0.405 mmol, 1 equiv) was purified by Prep-HPLC with the following conditions (Column: CHIRALPAK IH, 2*25 cm, 5 μm, Mobile Phase A: MTBE (0.1% TEA)—HPLC, Mobile Phase B: DCM:ACN=1:1—HPLC; Flow rate: 20 mL/min; Gradient: 60% B to 60% B in 15 min; Wave Length: 220/254 nm; RT1 (min): 5.82; RT2 (min): 9.82; The second peak was product) to afford Compound 232 (136.2 mg, 34.05%) as a yellow solid.

LCMS-Compound 232: (ES, m/z): [M+H]⁺ 987

NMR-Compound 232: (400 MHz, DMSO-d6, δ ppm): 1.52-1.54 (m, 2H), 1.86-1.96 (m, 3H), 2.08-2.18 (m, 2H), 2.34-2.40 (m, 1H), 2.51-2.54 (m, 2H), 2.60-2.69 (m, 2H), 2.82-2.89 (m, 1H), 3.16-3.23 (m, 4H), 3.24-3.29 (m, 4H), 3.30-3.41 (m, 12H), 3.53 (s, 2H), 3.84-3.87 (m, 1H), 4.17-4.22 (d, 1H), 4.30-4.34 (d, 1H), 4.49 (s, 2H), 5.02-5.07 (m, 1H), 7.02-7.09 (m, 4H), 7.26-7.31 (m, 4H), 7.41-7.53 (m, 3H), 7.65-7.69 (m, 2H), 8.15 (s, 1H), 8.39 (s, 1H), 10.99 (s, 1H).

### Example 231. Compound 233

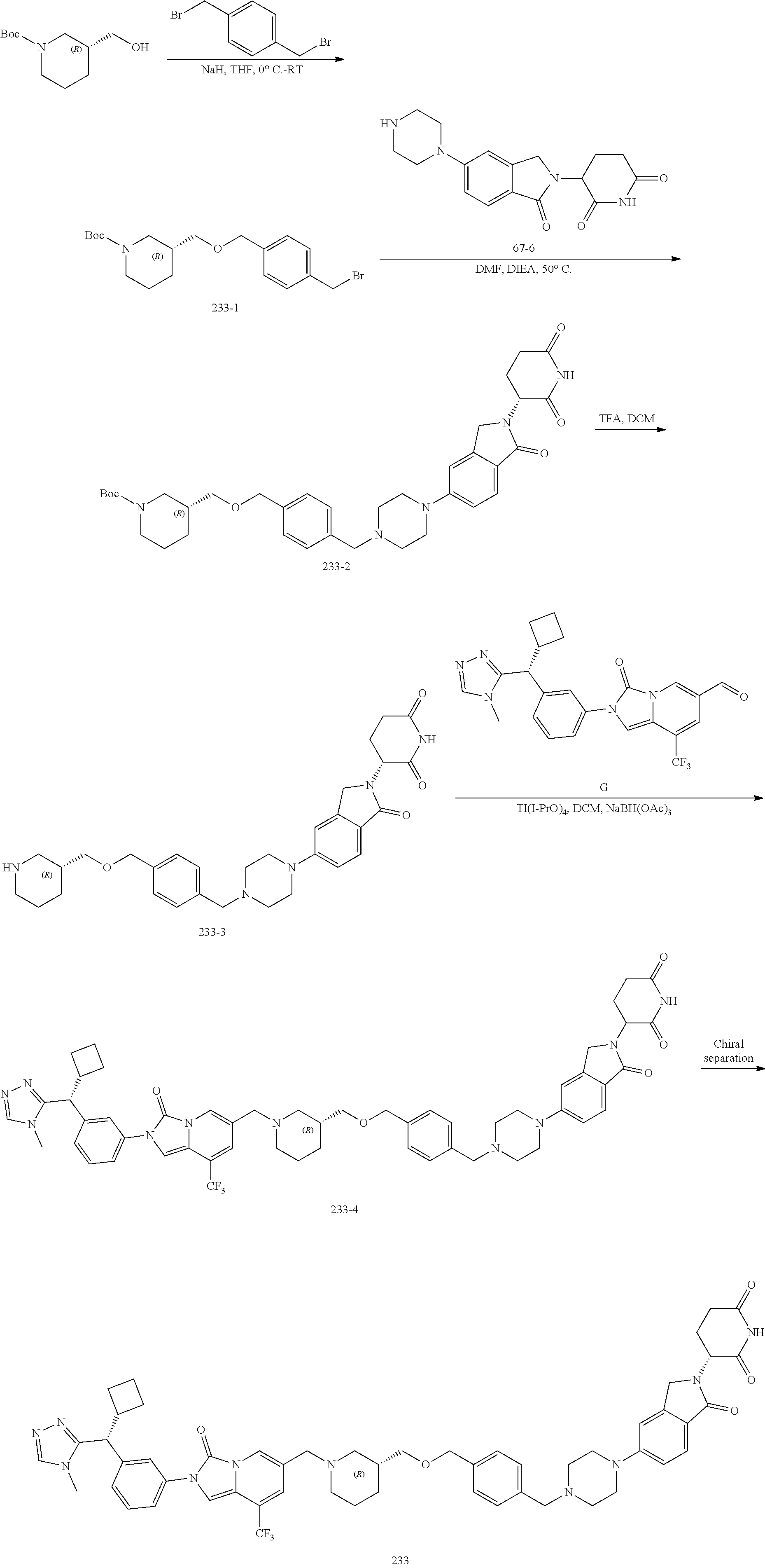

### Synthesis of Compound 233-1.

To a solution of tert-butyl (3R)-3-(hydroxymethyl)piperidine-1-carboxylate (1.5 g, 7.27 mmol, 1.2 equiv) in THF (16 mL) was added sodium hydride (60% in oil, 380 mg) at 0 degrees C. The mixture was stirred for 15 min. benzene, 1,4-bis(bromomethyl)-(1.6 g, 6.06 mmol, 1.0 equiv) was added and the mixture was allowed to warm to RT and stirred for 4 h. The crude product was used in the next step directly without further purification.

### Synthesis of Compound 233-2.

To a stirred solution of Compound 233-1 (1.6 g, 4.02 mmol, 1.0 equiv) and Compound 67-6 (1.9 g, 6.02 mmol, 1.5 equiv) in DMF (16 mL) was added DIEA (1.0 g, 8.03 mmol, 2.0 equiv) at room temperature. The resulting mixture was stirred for 4 h at room temperature under nitrogen atmosphere. The reaction was quenched by the addition of water/HOAc=10:1 (160 mL) at room temperature. The aqueous layer was extracted with CH₂Cl₂ (3×50 mL). The resulting mixture was concentrated under reduced pressure. The residue was purified by silica gel column chromatography, eluted with CH₂Cl₂/MeOH (80:1) to afford Compound 233-2 (1.6 g, 55%) as a off-white solid.

### Synthesis of Compound 233-3.

To a stirred solution of Compound 233-2 (1.5 g, 2.41 mmol, 1.0 equiv) in DCM (12 mL) were added TFA (3 mL) at room temperature. The resulting mixture was stirred for overnight at room temperature. The resulting mixture was diluted with CH₂Cl₂ (30 mL). The resulting mixture was concentrated under reduced pressure. The residue was purified by reversed-phase flash chromatography with the following conditions: column, C18 silica gel; mobile phase, MeCN in water (0.1% OAc), 10% to 50% gradient in 30 min; detector, UV 254 nm. This resulted in Compound 233-3 (900 mg, 67%) as a white solid.

### Synthesis of Compound 233-4.

To a stirred solution of Compound 233-3 (880 mg, 1.61 mmol, 1.0 equiv) and Intermediate G (881 mg, 1.94 mmol, 1.2 equiv) in DCM (9 mL) were added Ti(Oi-Pr)₄ (1833 mg, 6.45 mmol, 4.0 equiv) at room temperature. The resulting mixture was stirred for overnight at room temperature. To the above mixture was added NaBH(OAc)₃ (1025 mg, 4.84 mmol, 3.0 equiv) at room temperature. The resulting mixture was stirred for additional 3 h at room temperature. The reaction was quenched by the addition of water/HOAc=10:1 (100 mL) at room temperature. The aqueous layer was extracted with CH₂Cl₂/MeOH=5:1 (3×50 mL). The resulting mixture was concentrated under reduced pressure. The residue was purified by reversed-phase flash chromatography with the following conditions: column, C18 silica gel; mobile phase, MeCN in water (0.1% FA), 10% to 50% gradient in 30 min; detector, UV 254 nm. This resulted in Compound 233-4 (320 mg, 18%) as a yellow solid.

### Synthesis of Compound 233.

The Compound 233-4 (320 mg, 0.32 mmol, 1.0 equiv) was purified by Prep-Chiral-HPLC with the following conditions (Column: CHIRALPAK IH, 2*25 cm, 5 μm, Mobile Phase A: MTBE (0.1% TEA), Mobile Phase B: DCM:ACN=1:1; Flow rate: 20 mL/min; Gradient: 85% B to 85% B in 8 min; Wave Length: 220/254 nm; RT1 (min): 4.23; RT2 (min): 5.97; the first peak was product) to afford crude product. The crude product (110 mg) was purified by Prep-HPLC with the following conditions (Column: Sunfire prep C18 column, 30*150 mm, 5 μm; Mobile Phase A: Water (0.1% FA), Mobile Phase B: ACN; Flow rate: 60 mL/min; Gradient: 14% B to 27% B in 7 min, 27% B; Wave Length: 254; 220 nm; RT1 (min): 5.98; Number Of Runs: 0) to afford Compound 233 (52.4 mg, 16%) as a yellow solid.

LCMS-Compound 233: (ES, m/z): [M+H]⁺ 985

NMR-Compound 233: (400 MHz, DMSO-d6, δ ppm): 0.93-1.09 (m, 1H), 1.48-1.62 (m, 1H), 1.65-1.85 (m, 9H), 1.85-2.07 (m, 1H), 2.08-2.16 (m, 2H), 2.46-2.49 (m, 1H), 2.50-2.51 (m, 4H), 2.52-2.60 (m, 4H), 2.69-2.71 (m, 1H), 2.87-2.95 (m, 2H), 3.27-3.33 (m, 6H), 3.39-3.45 (m, 5H), 4.18-4.30 (m, 3H), 4.35-4.40 (m, 2H), 5.02-5.07 (m, 1H), 7.02-7.04 (m, 3H), 7.18-7.32 (m, 6H), 7.41-7.45 (t, 1H), 7.49-7.52 (d, 1H), 7.69-7.75 (m, 3H), 8.32 (s, 1H), 10.95 (s, 1H).

### Example 232. Compound 234

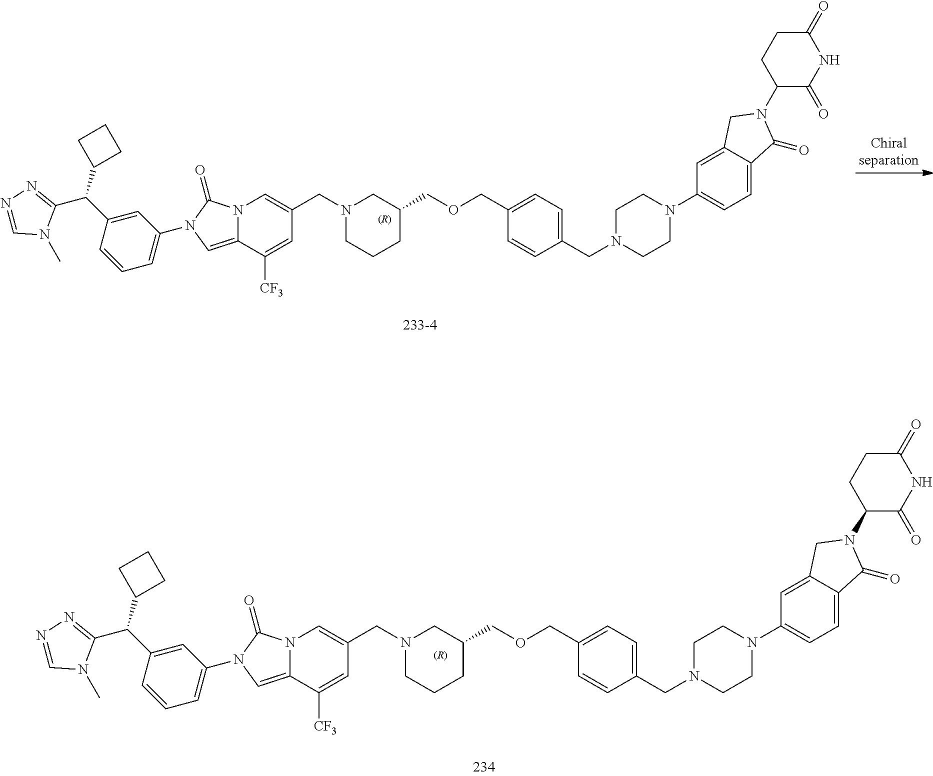

### Synthesis of Compound 234.

The Compound 233-4 (320 mg, 0.33 nmol, 1.0 equiv) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 85% B to 85% B in 8 min; Wave Length: 220/254 nm; RT1 (min): 4.23; RT2 (min): 5.97; the second peak was product) to afford crude product. The residue was purified by reversed-phase flash chromatography with the following conditions: column, C18 silica gel; mobile phase, MeCN in water (0.1% FA), 10% to 50% gradient in 25 min; detector, UV 254 nm. This resulted in Compound 234 (82.0 mg, 25%) as a yellow solid.

LCMS-Compound 234: (ES, m/z): [M+H]⁺ 985

NMR-Compound 234: (400 MHz, DMSO-d6, δ ppm): 0.93-1.05 (m, 1H), 1.41-1.51 (m, 1H), 1.61-1.95 (m, 9H), 1.96-2.02 (m, 3H), 2.32-2.45 (m, 1H), 2.50-2.51 (m, 4H), 2.60-2.67 (m, 4H), 2.82-2.84 (m, 1H), 2.90-2.99 (m, 2H), 3.25-3.31 (m, 6H), 3.20-3.44 (m, 5H), 4.17-4.40 (m, 5H), 5.03-5.07 (m, 1H), 7.02-7.04 (m, 3H), 7.18-7.41 (m, 6H), 7.43-7.52 (m, 2H), 7.68-7.76 (m, 3H), 8.33 (s, 1H), 10.95 (s, 1H).

### Example 233. Compound 235

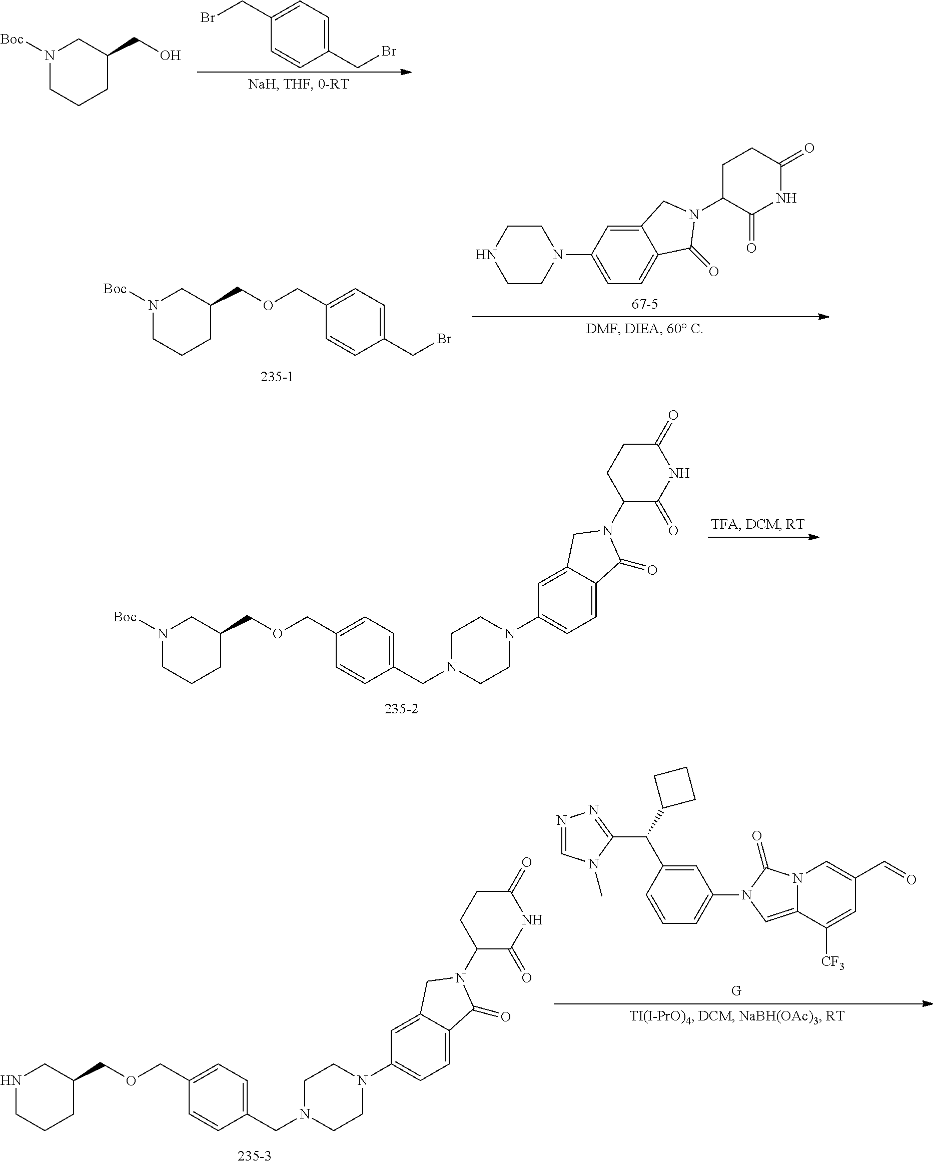

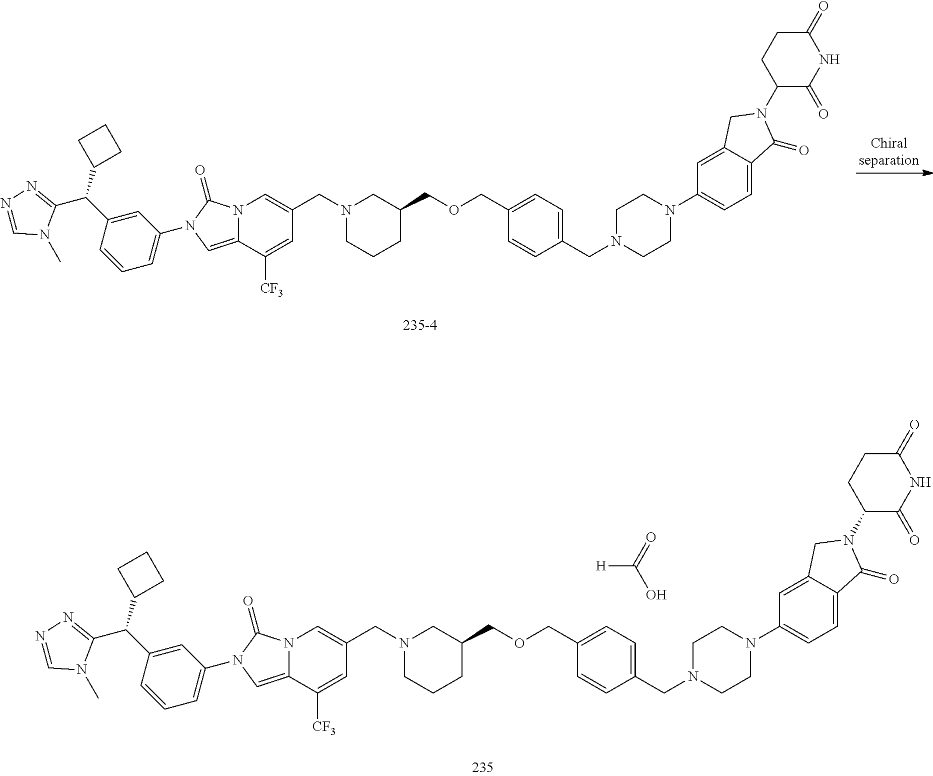 [SMILES: Cn1cnnc1[C@@H](c1cccc(-n2cc3c(C(F)(F)F)cc(CN4CCC[C@H](COCc5ccc(CN6CCN(c7ccc8c(c7)CN(C7CCC(=O)NC7=O)C8=O)CC6)cc5)C4)cn3c2=O)c1)C1CCC1 ; Cn1cnnc1[C@@H](c1cccc(-n2cc3c(C(F)(F)F)cc(CN4CCC[C@H](COCc5ccc(CN6CCN(c7ccc8c(c7)CN([C@@H]7CCC(=O)NC7=O)C8=O)CC6)cc5)C4)cn3c2=O)c1)C1CCC1 ; O=CO]

### Synthesis of Compound 235-1.

Into a 100 mL 3-necked round-bottom flask were added tert-butyl (38)-3-(hydroxymethyl) piperidine-1-carboxylate (2 g, 9.290 mmol, 1 equiv), THF (20 mL) and NaH (0.45 g, 11.251 mmol, 1.21 equiv, 60%) at 0° C. The resulting mixture was stirred for 1 h at 0° C. under nitrogen atmosphere. To the above mixture was added benzene, 1,4-bis(bromomethyl) (3.68 g, 13.942 mmol, 1.50 equiv) at 0° C. The resulting mixture was stirred for additional overnight at room temperature. The resulting mixture was concentrated under reduced pressure. The resulting mixture was used in the next step directly without further purification. This resulted in Compound 235-1 (2.5 g, 60.80%) as a light yellow oil.

### Synthesis of Compound 235-2.

Into a 100 mL 3-necked round-bottom flask were added Compound 235-1 (2.5 g, 6.276 mmol, 1 equiv), DMF (20 mL), Compound 67-5 (2.06 g, 6.276 mmol, 1 equiv) and DIEA (1.62 g, 12.552 mmol, 2 equiv) at room temperature. The resulting mixture was stirred for overnight at 60° C. under nitrogen atmosphere. The reaction was quenched by the addition of sat. NH₄Cl (aq.) (50 mL) at room temperature. The resulting mixture was extracted with EtOAc (3×50 mL). The combined organic layers were concentrated under reduced pressure. The residue was purified by silica gel column chromatography, eluted with CH₂Cl₂/MeOH (30:1) to afford Compound 235-2 (2.1 g, 48.70%) as an off-white solid.

### Synthesis of Compound 235-3.

Into a 100 mL 3-necked round-bottom flask were added TFA (4 mL), DCM (16 mL) and Compound 235-2 (2 g, 3.097 mmol, 1 equiv) at room temperature. The resulting mixture was stirred for overnight at room temperature. The resulting mixture was concentrated under reduced pressure. To afford Compound 235-3 (1.7 g, 94.56%) as a light yellow oil.

### Synthesis of Compound 235-4.

Into a 50 mL 3-necked round-bottom flask were added Compound 235-3 (1.7 g, 3.115 mmol, 1 equiv), DCM (30 mL). Intermediate G (1.84 g, 4.050 mmol, 1.3 equiv) and Ti(Oi-Pr)₄ (3.54 g, 12.460 mmol, 4 equiv) at room temperature. The resulting mixture was stirred for overnight at room temperature. To the above mixture was added NaBH(OAc)₃ (1.32 g, 6.230 mmol, 2 equiv) at room temperature. The resulting mixture was stirred for additional 3 h at room temperature. The reaction was quenched by the addition of sat. NH₄Cl (aq.) (100 mL) at room temperature. The resulting mixture was extracted with CH₂Cl₂ (3×200 mL). The combined organic layers were concentrated under reduced pressure. The residue was purified by reversed-phase flash chromatography with the following conditions: column, C18 silica gel; mobile phase, MeCN in water (0.1% FA), 10% to 50% gradient in 30 min; detector, UV 254 nm. To afford Compound 235-4 (1.8 g, 55.72%) as a light yellow solid.

### Synthesis of Compound 235.

The Compound 235-4 (440 mg) was purified by Prep-Chiral-HPLC with the following conditions (Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 60% B to 60% B in 13 min; Wave Length: 220/254 nm; RT1 (min): 6.70; RT2 (min): 11.19; The first peak was the product. Sample Solvent: DCM:ACN=1:1—HPLC; Injection Volume: 0.9 mL; Number of Runs: 5) to afford crude product. The crude product was purified by reversed-phase flash chromatography with the following conditions: column, 40 g C18 silica gel; mobile phase, MeCN in water (0.1% FA), 20% to 55% gradient in 8 min; detector, UV 254 nm to afford Compound 235 (83.6 mg, 18.48%) as a light yellow solid.

LCMS-Compound 235: (ES, m/z): [M−HCOOH+H]⁺ 985

H-NMR-Compound 235: (300 MHz, CD₃OD, ppm): δ0.95-1.05 (m, 1H), δ1.40-1.52 (m, 1H), δ1.61-1.82 (m, 9H), δ1.95-2.11 (m, 3H), δ2.33-2.38 (m, 2H), δ2.38-2.49 (m, 5H), δ2.50-2.60 (m, 1H), δ2.60-2.89 (m, 3H), δ3.25-3.27 (m, 7H), δ3.42-3.43 (m, 5H), δ4.17-4.39 (m, 5H), δ5.02-5.06 (m, 1H), δ7.01-7.04 (m, 3H), δ7.17-7.32 (m, 6H), δ7.32-7.52 (m, 2H), δ7.67-7.75 (m, 3H), δ8.32 (s, 1H), δ10.93 (s, 1H).

### Example 234. Compound 236

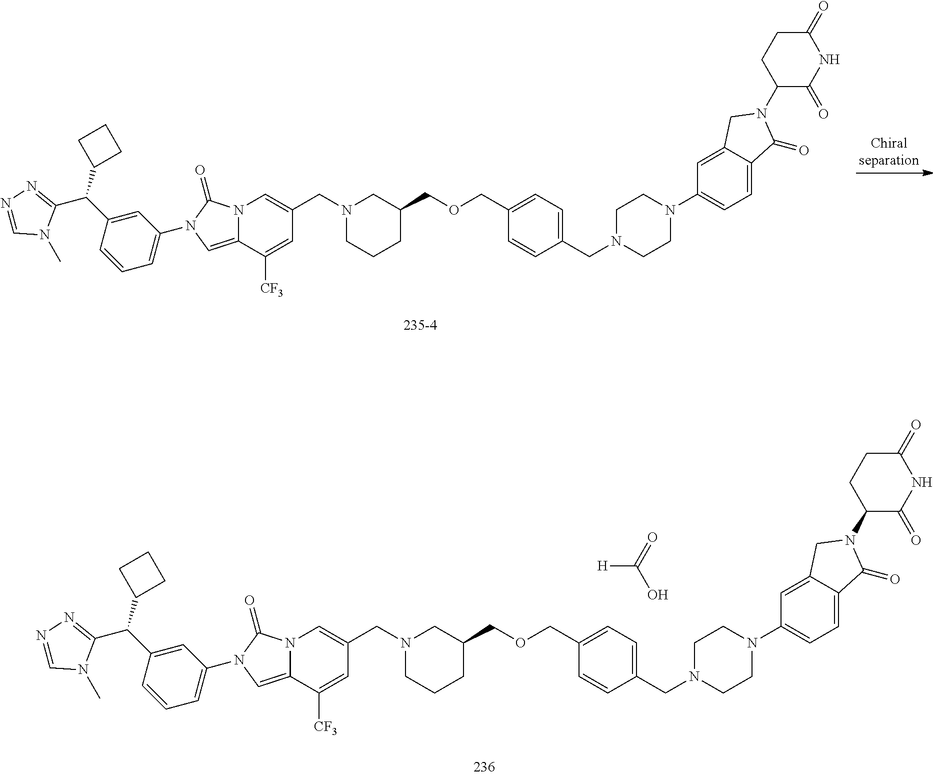 [SMILES: Cn1cnnc1[C@@H](c1cccc(-n2cc3c(C(F)(F)F)cc(CN4CCC[C@H](COCc5ccc(CN6CCN(c7ccc8c(c7)CN(C7CCC(=O)NC7=O)C8=O)CC6)cc5)C4)cn3c2=O)c1)C1CCC1 ; Cn1cnnc1[C@@H](c1cccc(-n2cc3c(C(F)(F)F)cc(CN4CCC[C@H](COCc5ccc(CN6CCN(c7ccc8c(c7)CN([C@H]7CCC(=O)NC7=O)C8=O)CC6)cc5)C4)cn3c2=O)c1)C1CCC1 ; O=CO]

### Synthesis of Compound 236.

The Compound 235-4 (440 mg) was purified by Prep-Chiral-HPLC with the following conditions (Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 60% B to 60% B in 13 min; Wave Length: 220/254 nm; RT1 (min): 6.70; RT2 (min): 11.19; The second peak was the product. Sample Solvent: DCM:ACN=1:1—HPLC; Injection Volume: 0.9 mL; Number of Runs: 5) to afford crude product. The crude product was purified by reversed-phase flash chromatography with the following conditions: column, 40 g C18 silica gel; mobile phase, MeCN in water (0.1% FA), 20% to 55% gradient in 8 min; detector, UV 254 nm to afford Compound 236 (101.3 mg, 22.11%) as a light yellow solid.

LCMS-Compound 236: (ES, m/z): [M−HCOOH+H]⁺ 985

H-NMR-Compound 236: (300 MHz, CD₃OD, ppm): δ0.95-1.05 (m, 1H), δ1.40-1.52 (m, 1H), δ1.61-1.95 (m, 9H), δ1.96-2.07 (m, 3H), δ2.33-2.44 (m, 1H), δ2.44-2.49 (m, 4H), δ2.50-2.60 (m, 2H), δ2.82-2.90 (m, 2H), δ3.25-3.28 (m, 9H), δ3.34-3.44 (m, 5H), δ4.17-4.34 (m, 3H), δ4.34-4.40 (m, 2H), δ5.02-5.06 (m, 1H), δ7.01-7.04 (m, 3H), δ7.17-7.32 (m, 6H), δ7.32-7.52 (m, 2H), δ7.67-7.75 (m, 3H), δ8.32 (s, 1H), δ10.93 (s, 1H).

### Example 235. Compound 237

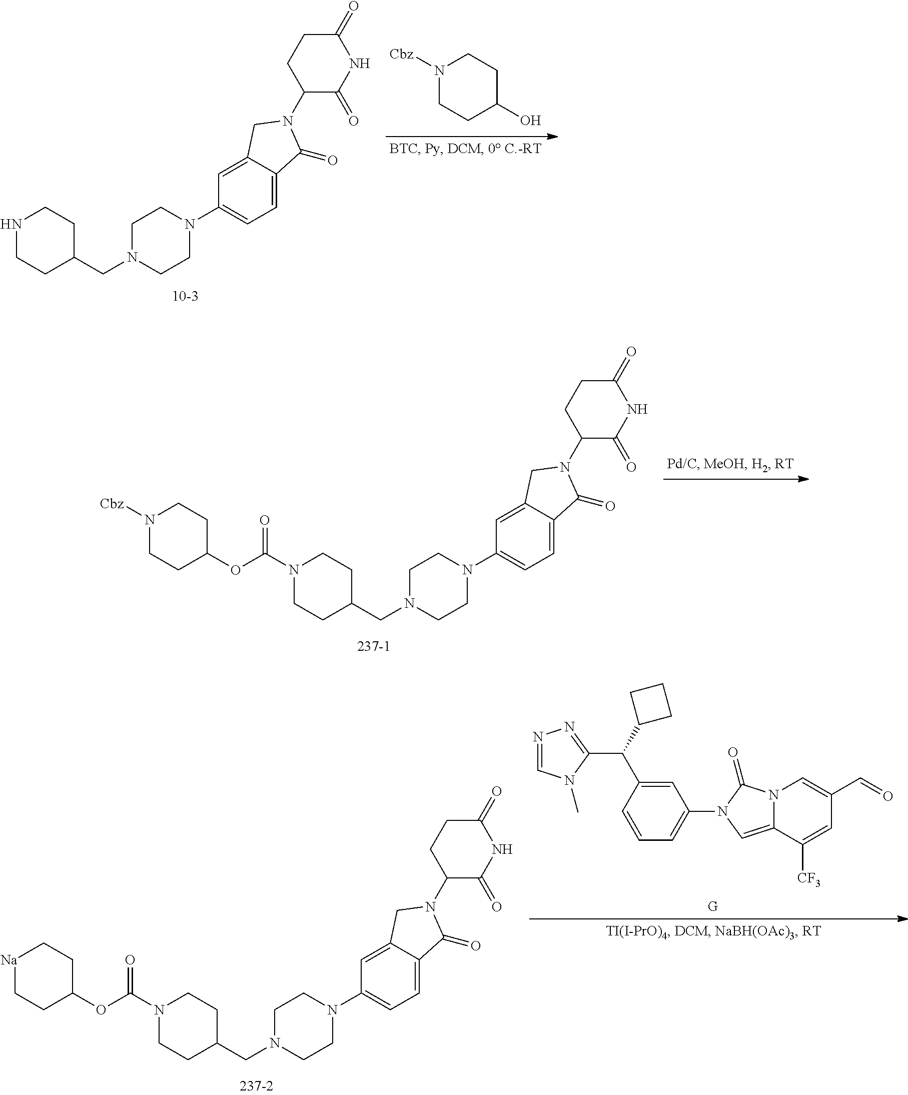

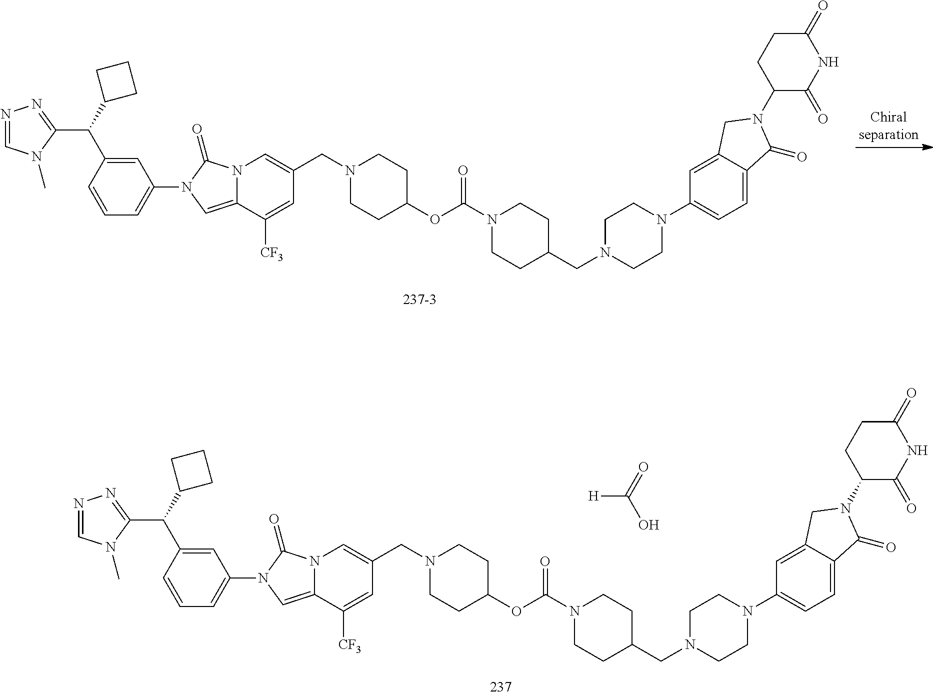 [SMILES: Cn1cnnc1[C@@H](c1cccc(-n2cc3c(C(F)(F)F)cc(CN4CCC(OC(=O)N5CCC(CN6CCN(c7ccc8c(c7)CN(C7CCC(=O)NC7=O)C8=O)CC6)CC5)CC4)cn3c2=O)c1)C1CCC1 ; Cn1cnnc1[C@@H](c1cccc(-n2cc3c(C(F)(F)F)cc(CN4CCC(OC(=O)N5CCC(CN6CCN(c7ccc8c(c7)CN([C@@H]7CCC(=O)NC7=O)C8=O)CC6)CC5)CC4)cn3c2=O)c1)C1CCC1 ; O=CO]

### Synthesis of Compound 237-1.

Into a 100 mL 3-necked round-bottom flask were added benzyl 4-hydroxypiperidine-1-carboxylate (1.6 g, 6.800 mmol, 1 equiv), DCM (30 mL) and pyridine (2.69 g, 34,000 mmol, 5 equiv) at room temperature. To the above mixture was added triphosgene (0.81 g, 2.720 mmol, 0.4 equiv) at 0° C. The resulting mixture was stirred for additional 1 h at 0° C. To the above mixture was added Compound 10-3 (2.00 g, 4.692 mmol, 0.69 equiv) at 0° C. The resulting mixture was stirred for additional overnight at room temperature. The reaction was quenched by the addition of sat. NH₄Cl (aq.) (20 mL) at room temperature. The resulting mixture was extracted with CH₂Cl₂ (3×20 mL). The combined organic layers were concentrated under reduced pressure. The residue was purified by Prep-TLC (CH₂Cl₂/MeOH 20:1) to afford Compound 237-1 (900 mg, 18.11%) as a light yellow solid.

### Synthesis of Compound 237-2.

To a solution of Compound 237-1 (1.8 g, 2.621 mmol, 1 equiv) in 50 mL MeOH was added Pd/C (10%, 181.29 mg) under nitrogen atmosphere in a 100 mL round-bottom flask. The mixture was hydrogenated at room temperature for 1 h under hydrogen atmosphere using a hydrogen balloon, filtered through a Celite pad and concentrated under reduced pressure. To afford Compound 237-2 (1.1 g, 72.15%) as a light yellow solid.

### Synthesis of Compound 237-3.

Into a 40 mL sealed tube were added Compound 237-2 (1.2 g, 2.171 mmol, 1 equiv), DCM (20 mL) Intermediate G (1.29 g, 2.822 mmol, 1.3 equiv) and Ti(Oi-Pr)₄ (2.47 g, 8.684 mmol, 4 equiv) at room temperature. The resulting mixture was stirred for overnight at room temperature. To the above mixture was added NaBH(OAc)₄ (0.92 g, 4.342 mmol, 2 equiv) at room temperature. The resulting mixture was stirred for additional 3 h at room temperature. The reaction was quenched by the addition of sat. NH₄Cl (aq.) (100 mL) at room temperature. The resulting mixture was extracted with CH₂Cl₂ (3×200 mL). The combined organic layers were concentrated under reduced pressure. The residue was purified by reversed-phase flash chromatography with the following conditions: column, C18 silica gel; mobile phase, MeCN in water (0.1% FA), 10% to 50% gradient in 30 min; detector, UV 254 nm. This resulted in Compound 237-3 (500 mg, 22.75%) as a light yellow solid.

### Synthesis of Compound 237.

The Compound 237-3 (450 mg) was purified by Prep-Chiral-HPLC with the following conditions (Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 60% B to 60% B in 15 min; Wave Length: 220/254 nm; RT1 (min): 7.68; RT2 (min): 12.90; The first peak was the product. Sample Solvent: DCM:ACN=1:1—HPLC; Injection Volume: 0.9 mL; Number of Runs: 6) to afford crude product. The crude product was purified by reversed-phase flash chromatography with the following conditions: column, 40 g C18 silica gel; mobile phase, MeCN in water (0.1% FA), 20% to 55% gradient in 8 min; detector, UV 254 nm to afford Compound 237 (103.6 mg, 23.07%) as a light yellow solid.

LCMS-Compound 237: (ES, m/z): [M−HCOOH+H]⁺ 992

H-NMR-Compound 237: (300 MHz, CD₃OD, ppm): δ0.96-1.05 (m, 2H), δ1.57-1.58 (m, 2H), δ1.69-1.80 (m, 1H), δ1.95-2.16 (m, 3H), δ2.18-2.32 (m, 3H), δ2.32-2.49 (m, 4H), δ2.56-2.60 (m, 3H), δ2.60-2.90 (m, 4H), δ3.27-3.29 (m, 6H), δ3.42 (s, 3H), δ3.96-3.99 (m, 2H), δ4.17-4.35 (m, 3H), δ4.59 (s, 1H), δ5.02-5.07 (m, 1H), δ7.02-7.06 (m, 3H), δ7.18-7.41 (m, 2H), δ7.41-7.45 (m, 1H), δ7.45-7.53 (m, 1H), δ7.68-7.73 (m, 3H), δ8.32 (s, 1H), δ10.96 (s, 1H).

### Example 236. Compound 238

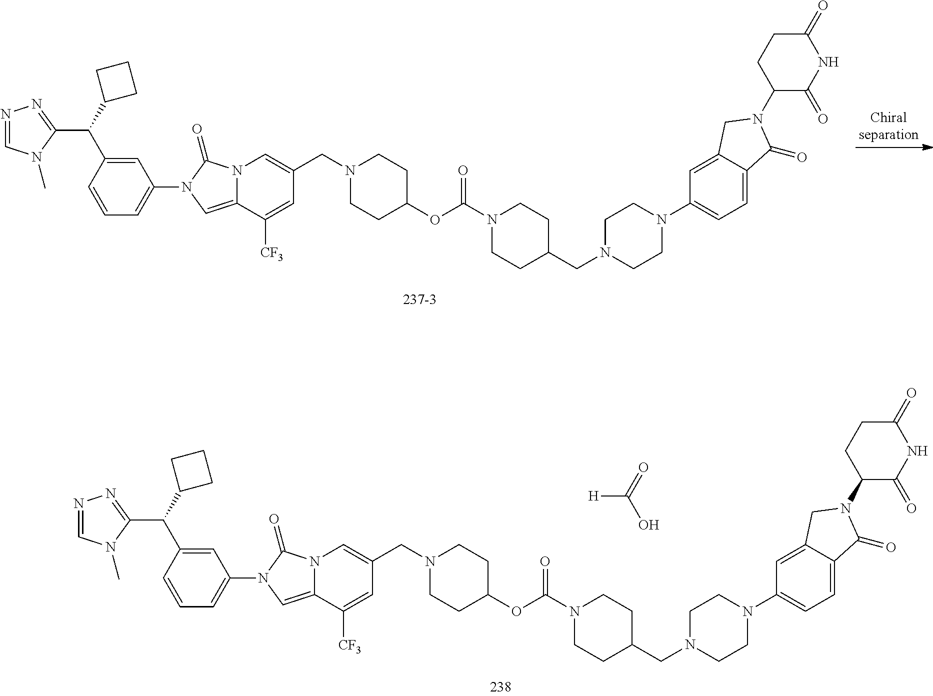 [SMILES: Cn1cnnc1[C@@H](c1cccc(-n2cc3c(C(F)(F)F)cc(CN4CCC(OC(=O)N5CCC(CN6CCN(c7ccc8c(c7)CN(C7CCC(=O)NC7=O)C8=O)CC6)CC5)CC4)cn3c2=O)c1)C1CCC1 ; Cn1cnnc1[C@@H](c1cccc(-n2cc3c(C(F)(F)F)cc(CN4CCC(OC(=O)N5CCC(CN6CCN(c7ccc8c(c7)CN([C@H]7CCC(=O)NC7=O)C8=O)CC6)CC5)CC4)cn3c2=O)c1)C1CCC1 ; O=CO]

### Synthesis of Compound 238.

The Compound 237-3 (450 mg) was purified by Prep-Chiral-HPLC with the following conditions (Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 60% B to 60% B in 15 min; Wave Length: 220/254 nm; RT1 (min): 7.68; RT2 (min): 12.90; The second peak was the product. Sample Solvent: DCM:ACN=1:1—HPLC; Injection Volume: 0.9 mL; Number of Runs: 6) to afford crude product. The crude product was purified by reversed-phase flash chromatography with the following conditions: column, 40 g C18 silica gel; mobile phase, MeCN in water (0.1% FA), 20% to 55% gradient in 8 min; detector, UV 254 nm to afford Compound 238 (69.0 mg, 15.36%) as a light yellow solid.

LCMS-Compound 238: (ES, m/z): [M−HCOOH+H]⁺ 992

H-NMR-Compound 238: (300 MHz, CD₃OD, ppm): δ0.96-1.05 (m, 2H), δ1.56-1.58 (m, 2H), δ1.69-1.95 (m, 10H), δ1.95-1.99 (m, 1H), δ2.01-2.05 (m, 1H), δ2.05-2.17 (m, 2H), δ2.20-2.49 (m, 4H), δ2.51-2.59 (m, 4H), δ2.59-2.95 (m, 3H), δ3.21-3.30 (m, 8H), δ3.42 (s, 4H), δ3.96-3.99 (m, 2H), δ4.17-4.34 (m, 3H), δ4.59 (s, 1H), δ7.02-7.06 (m, 1H), δ7.18-7.41 (m, 3H), δ7.41-7.45 (m, 1H), δ7.45-7.52 (m, 1H), δ7.52-7.67 (m, 1H), δ7.69-7.73 (m, 3H), δ8.32 (s, 1H), δ10.96 (s, 1H).

### Example 237. Compound 239

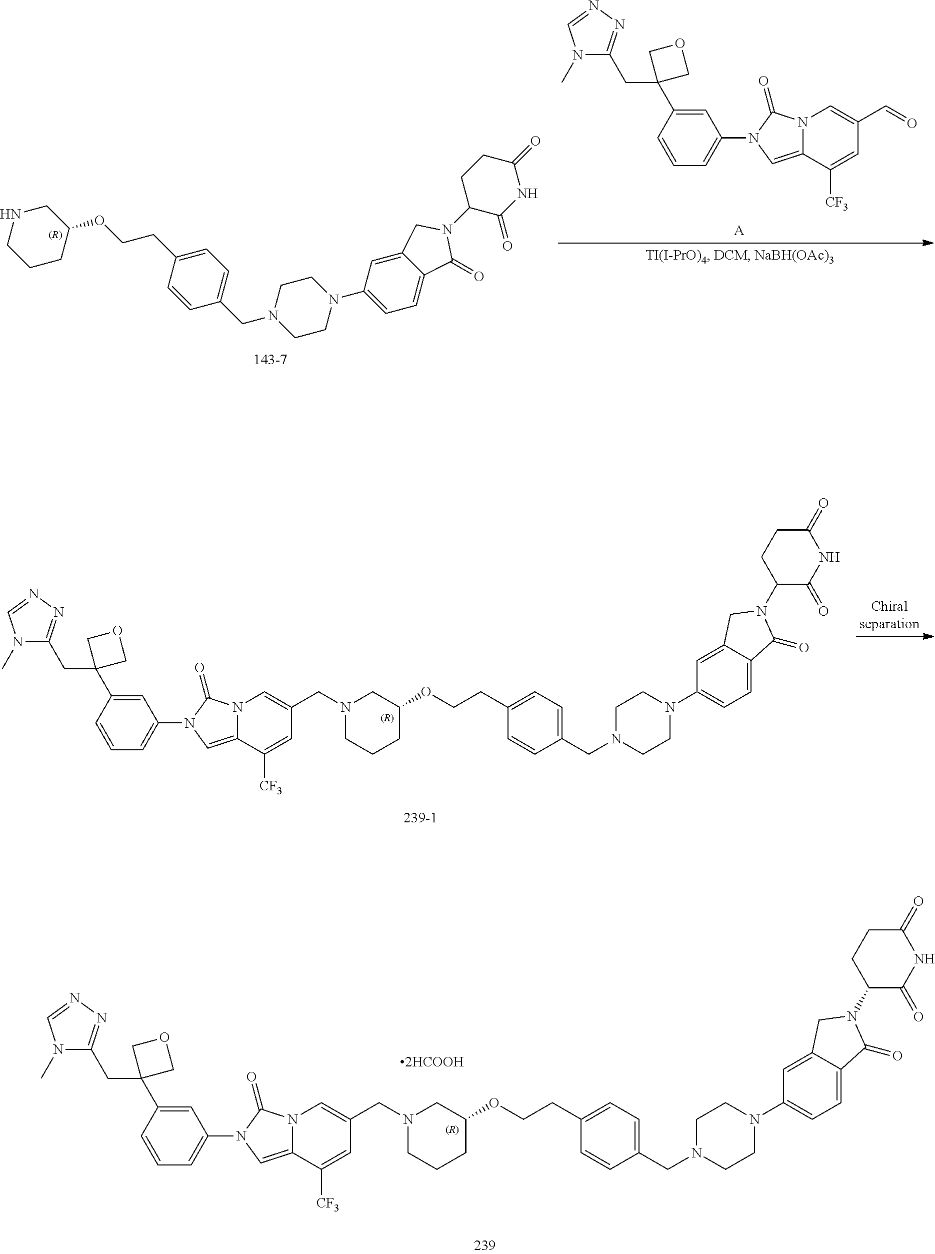

### Synthesis of Compound 239-1.

To a stirred solution of Compound 143-7 (0.8 g, 1.466 mmol, 1 equiv) and Intermediate A (0.80 g, 1.759 mmol, 1.2 equiv) in DCM (30 mL) was added Ti(OEt)₄ (1.00 g, 4.398 mmol, 3 equiv) at room temperature. The resulting mixture was stirred overnight at room temperature. To the above mixture was added NaBH(OAc)₃ (0.93 g, 4.398 mmol, 3 equiv) at room temperature. The resulting mixture was stirred for additional 6 h at room temperature. The reaction was quenched with 10% HOAc (aq.) at room temperature. The aqueous layer was extracted with EtOAc (80 mL). The aqueous layer was filtered, the filter cake was washed with water (10 mL). The filtrate was concentrated under reduced pressure. The residue was purified by reverse flash chromatography with the following conditions: column, C18 silica gel; mobile phase, MeCN in water (0.1% FA), 10% to 50% gradient in 20 min; detector, UV 254 nm. This resulted in Compound 239-1 (480 mg, 30.85%) as a yellow solid.

### Synthesis of Compound 239.

The Compound 239-1 (480 ng) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 90% B to 90% B in 12 min; Wave Length: 220/254 nm; RT1 (min): 6.28; RT2 (min): 9.48; the first peak is product) to afford the crude product. The residue was purified by Prep-HPLC with the following conditions (Column: Sunfire prep C18 column, 30*150 mm, 5 μm; Mobile Phase A: Water (0.1% FA), Mobile Phase B: ACN; Flow rate: 60 mL/min; Gradient: 8% B to 18% B in 10 min, Wave Length: 254; 220 nm; RT1 (min): 9.7) to afford Compound 239 (78.8 mg, 14.94%) as a yellow solid.

LC-MS-Compound 239: (ES, m/z): [M−2HCOOH+H]⁺ 987

H-NMR-Compound 239: (400 MHz, DMSO-d₆, δ ppm): 1.05-1.18 (m, 1H), 1.31-1.45 (m, 1H), 1.61-1.71 (m, 1H), 1.85-2.02 (m, 4H), 2.31-2.42 (m, 1H), 2.45-2.50 (m, 4H), 2.55-2.69 (m, 2H), 2.73-2.77 (m, 2H), 2.83-2.94 (m, 2H), 2.96 (s, 3H), 3.26-3.35 (m, 7H), 3.46 (s, 4H), 3.52 (s, 2H), 3.58-3.61 (m, 2H), 4.16-4.20 (m, 1H), 4.29-4.33 (m, 1H), 4.90-4.95 (m, 4H), 5.01-5.05 (m, 1H), 6.87-6.89 (d, 1H), 7.01-7.03 (m, 3H), 7.17-7.22 (m, 4H), 7.32-7.41 (m, 3H), 7.49-7.51 (d, 1H), 7.67 (s, 1H), 7.74-7.77 (d, 1H), 8.15 (s, 2H), 8.19 (s, 1H), 10.95 (s, 1H).

### Example 238. Compound 240

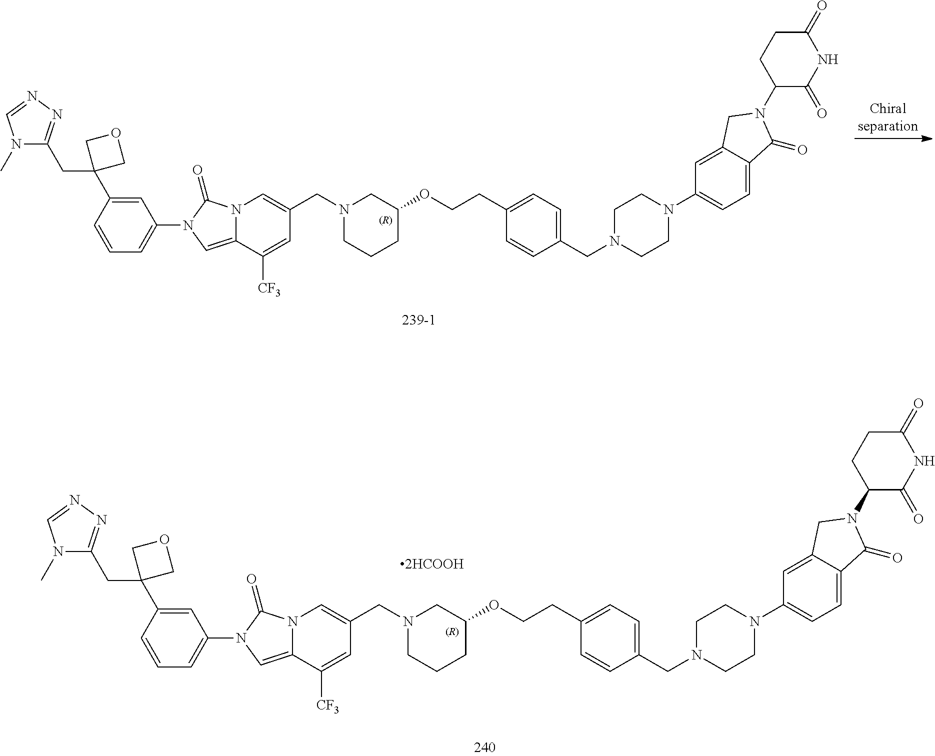

### Synthesis of Compound 240.

The Compound 239-1 (480 mg) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 90% B to 90% B in 12 min; Wave Length: 220/254 nm; RT1 (min): 6.28; RT2 (min, the second peak is product) to afford the crude product. The residue was purified by Prep-HPLC with the following conditions (Column: Sunfire prep C18 column, 30*150 mm, 5 μm; Mobile Phase A: Water (0.1% FA), Mobile Phase B: ACN; Flow rate: 60 mL/min; Gradient: 8% B to 18% B in 10 min, Wave Length: 254; 220 nm; RT1 (min): 9.88) to afford Compound 240 (78.8 mg, 14.94%) as a yellow solid.

LC-MS-Compound 240: (ES, m/z): [M−2HCOOH+H]⁺ 987

H-NMR-Compound 240: (400 MHz, DMSO-d₆, δ ppm): 1.05-1.18 (m, 1H), 1.31-1.45 (m, 1H), 1.61-1.71 (m, 1H), 1.85-2.02 (m, 4H), 2.31-2.42 (m, 1H), 2.45-2.50 (m, 4H), 2.55-2.69 (m, 2H), 2.73-2.77 (m, 2H), 2.83-2.94 (m, 2H), 2.96 (s, 3H), 3.26-3.35 (m, 7H), 3.46 (s, 4H), 3.52 (s, 2H), 3.58-3.61 (m, 2H), 4.16-4.20 (m, 1H), 4.29-4.33 (m, 1H), 4.90-4.95 (m, 4H), 5.01-5.05 (m, 1H), 6.87-6.89 (d, 1H), 7.01-7.03 (m, 3H), 7.17-7.22 (m, 4H), 7.32-7.41 (m, 3H), 7.49-7.51 (d, 1H), 7.67 (s, 1H), 7.74-7.77 (d, 1H), 8.15 (s, 2H), 8.19 (s, 1H), 10.95 (s, 1H).

### Example 239. Compound 241

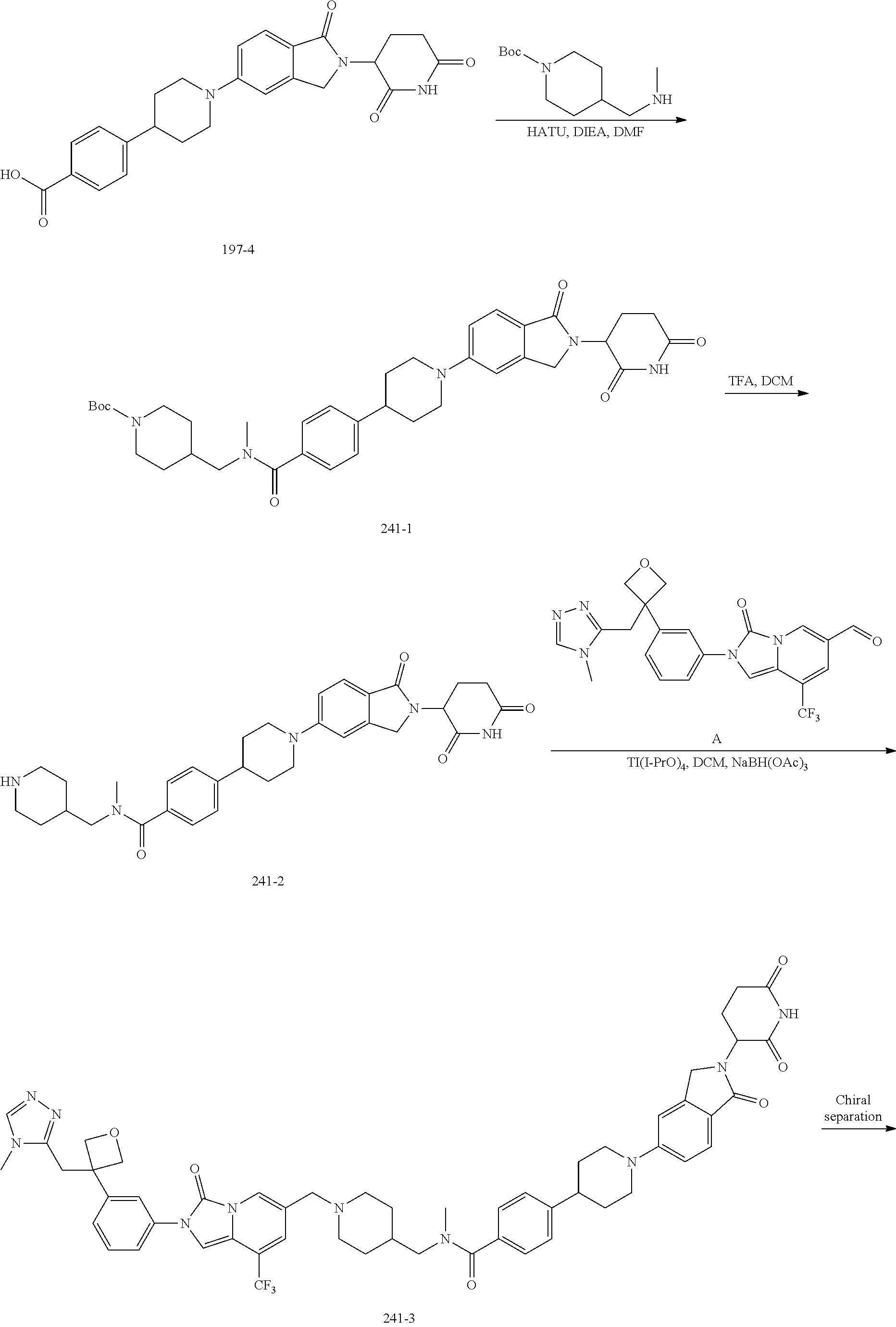

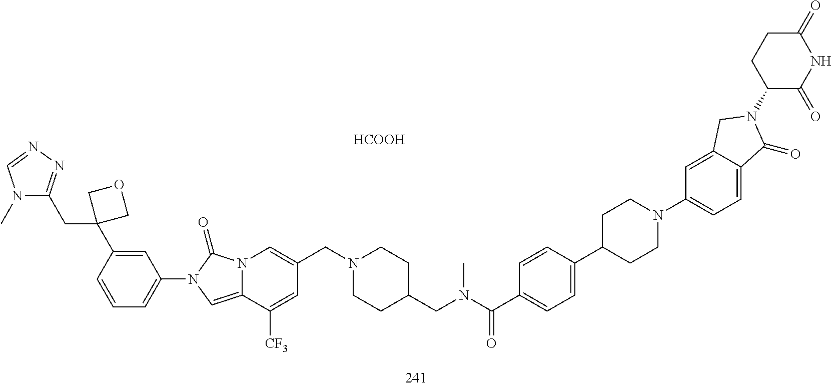 [SMILES: CN(CC1CCN(Cc2cc(C(F)(F)F)c3cn(-c4cccc(C5(Cc6nncn6C)COC5)c4)c(=O)n3c2)CC1)C(=O)c1ccc(C2CCN(c3ccc4c(c3)CN([C@@H]3CCC(=O)NC3=O)C4=O)CC2)cc1 ; "HCOOH"]

### Synthesis of Compound 241-1.

A solution of Compound 197-4 (1.0 g, 2.23 mmol, 1.0 equiv) in DMF (10 mL) was treated with HATU (1.3 g, 3.35 mmol, 1.5 equiv) and DIEA (870 mg, 6.71 mmol, 3.0 equiv) for 1 h at room temperature under nitrogen atmosphere followed by the addition of tert-butyl 4-{[1-(4-{1-[2-(2,6-dioxopiperidin-3-yl)-1-oxo-3H-isoindol-5-yl]piperidin-4-yl}phenyl)-N-methylformamido]methyl}piperidine-1-carboxylate (1.3 g, 88.44%) at room temperature. The reaction was quenched by the addition of water/HOAc=10:1 (30 mL) at room temperature. The resulting mixture was extracted with CH₂Cl₂/MeOH=10:1 (3×20 mL). The combined organic layers were concentrated under reduced pressure. The residue was purified by silica gel column chromatography, eluted with CH₂Cl₂/MeOH (25:1) to afford Compound 241-1 (1.3 g, 88%) as a yellow solid.

### Synthesis of Compound 241-2.

To a stirred solution of Compound 241-1 (1.3 g, 1.98 mmol, 1.0 equiv) in DCM (10 ml) were added TFA (2 mL) at room temperature under nitrogen atmosphere. The resulting mixture was stirred for overnight at room temperature under nitrogen atmosphere. The resulting mixture was concentrated under reduced pressure. The residue was purified by reversed-phase flash chromatography with the following conditions: column, C18 silica gel; mobile phase, MeCN in water (0.1% FA), 0% to 40% gradient in 30 min; detector, UV 254 nm. This resulted in Compound 241-2 (730 mg, 66%) as a white solid.

### Synthesis of Compound 241-3.

To a stirred solution of Compound 241-2 (720 mg, 1.29 mmol, 1.0 equiv) and Intermediate A (709 mg, 1.55 mmol, 1.2 equiv) in DCM (8 mL) were added Ti(Oi-Pr)₄ (1467 mg, 5.16 mmol, 4.0 equiv) at room temperature under nitrogen atmosphere. The resulting mixture was stirred for overnight at room temperature under nitrogen atmosphere. To the above mixture was added NaBH(OAc)₃ (821 mg, 3.87 mmol, 3.0 equiv) at room temperature. The resulting mixture was stirred for additional 4 h at room temperature. The reaction was quenched by the addition of water/HOAc=10:1 (80 mL) at room temperature. The aqueous layer was extracted with CH₂Cl₂/MeOH=5:1 (3×50 mL). The resulting mixture was concentrated under reduced pressure. The residue was purified by reversed-phase flash chromatography with the following conditions: column, C18 silica gel; mobile phase, MeCN in water (0.1% FA), 10% to 50% gradient in 30 min; detector, UV 254 nm. The crude product (400 mg) was purified by Prep-HPLC with the following conditions (Column: Sunfire prep C18 column, 30*150 mm, 5 μm; Mobile Phase A: Water (0.1% FA), Mobile Phase B: ACN; Flow rate: 60 mL/min; Gradient: 18% B to 28% B in 8 min, 28% B; Wave Length: 254; 220 nm; RT1 (min): 7.57) to afford Compound 241-3 (220 mg, 16%) as a yellow solid.

### Synthesis of Compound 241.

The Compound 241-3 (220 mg, 0.22 mmol, 1.0 equiv) was purified by Prep-Chiral-HPLC with the following conditions (Column: CHIRALPAK IH, 2*25 cm, 5 μm; Mobile Phase A: Hex (0.2% TEA), Mobile Phase B: IPA:DCM=1:1; Flow rate: 20 mL/min; Gradient: 90% B to 90% B in 10 min; Wave Length: 220/254 nm; RT1 (min): 3.92; RT2 (min): 7.36; the first peak was product) to afford crude product. The residue was purified by reversed-phase flash chromatography with the following conditions: column, C18 silica gel; mobile phase, MeCN in water (0.1% FA), 10% to 50% gradient in 25 min; detector, UV 254 nm. This resulted in Compound 241 (77.1 mg, 33.20%) as a yellow solid.

LCMS-Compound 241: (ES, m/z): [M−HCOOH+H]⁺ 999

NMR-Compound 241: (400 MHz, DMSO-d6, δ ppm): 0.73.89 (m, 1H), 1.13-1.29 (m, 2H), 1.31-1.52 (m, 1H), 1.55-1.78 (m, 4H), 1.79-1.97 (m, 5H), 2.36-2.49 (m, 1H), 2.51-2.61 (m, 1H), 2.82-2.96 (m, 12H), 2.97-3.16 (m, 1H), 3.17-3.22 (m, 1H), 3.23-3.34 (m, 2H), 3.35-3.53 (m, 2H), 4.01-4.04 (m, 2H), 4.19-4.23 (d, 1H), 4.31-4.36 (d, 1H), 4.90-5.03 (m, 4H), 5.04-5.07 (m, 1H), 6.88-7.10 (m, 2H), 7.12-7.32 (t, 2H), 7.33-7.38 (m, 5H), 7.40-7.42 (m, 2H), 7.51-7.53 (m, 1H), 7.59-7.69 (m, 1H), 7.74-7.76 (d, 1H), 8.16-8.20 (m, 2H), 10.95 (s, 1H).

### Example 240. Compound 242

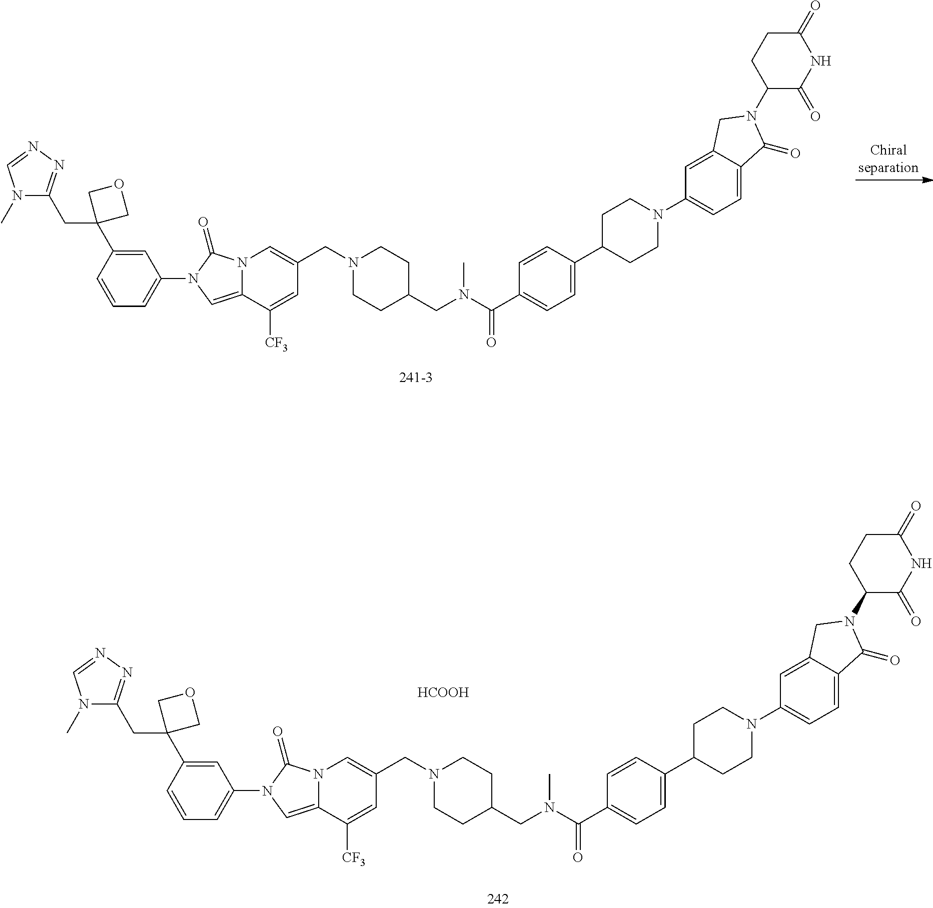 [SMILES: CN(CC1CCN(Cc2cc(C(F)(F)F)c3cn(-c4cccc(C5(Cc6nncn6C)COC5)c4)c(=O)n3c2)CC1)C(=O)c1ccc(C2CCN(c3ccc4c(c3)CN(C3CCC(=O)NC3=O)C4=O)CC2)cc1 ; CN(CC1CCN(Cc2cc(C(F)(F)F)c3cn(-c4cccc(C5(Cc6nncn6C)COC5)c4)c(=O)n3c2)CC1)C(=O)c1ccc(C2CCN(c3ccc4c(c3)CN([C@H]3CCC(=O)NC3=O)C4=O)CC2)cc1 ; "HCOOH"]

### Synthesis of Compound 242.

The Compound 241-3 (220 mg, 0.22 mmol, 1.0 equiv) was purified by Prep-Chiral-HPLC with the following conditions (Column: CHIRALPAK IH, 2*25 cm, 5 μm; Mobile Phase A: Hex (0.2% TEA), Mobile Phase B: IPA:DCM=1:1; Flow rate: 20 mL/min; Gradient: 90% B to 90% B in 10 min; Wave Length: 220/254 nm; RT1 (min): 3.92; RT2 (min): 7.36; the second peak was product) to afford crude product. The residue was purified by reversed-phase flash chromatography with the following conditions: column, C18 silica gel; mobile phase, MeCN in water (0.1% FA), 10% to 50% gradient in 25 min; detector, UV 254 nm. This resulted in Compound 242 (78.8 mg, 34%) as a yellow solid.

LCMS-Compound 242: (ES, m/z): [M−HCOOH+H]⁺ 999

NMR-Compound 242: (400 MHz, DMSO-d6, δ ppm): 0.73-1.01 (m, 1H), 1.15-1.33 (m, 1H), 1.35-1.88 (m, 5H), 1.91-2.13 (m, 5H), 2.32-2.49 (m, 1H), 2.50-2.61 (m, 1H), 2.82-2.97 (m, 12H), 3.16-3.21 (m, 2H), 3.22-3.30 (m, 2H), 3.34-3.53 (m, 2H), 4.01-4.04 (m, 2H), 4.19-4.23 (d, 1H), 4.32-4.36 (d, 1H), 4.90-4.96 (m, 4H), 5.03-5.07 (m, 1H), 6.88-6.90 (d, 1H), 6.92-7.09 (m, 1H), 7.10-7.12 (m, 2H), 7.32-7.38 (m, 5H), 7.40-7.42 (t, 2H), 7.51-7.53 (d, 1H), 7.54-7.73 (m, 1H), 7.74-7.76 (d, 1H), 8.17-7.19 (m, 2H), 10.96 (s, 1H).

### Example 241. Compound 243

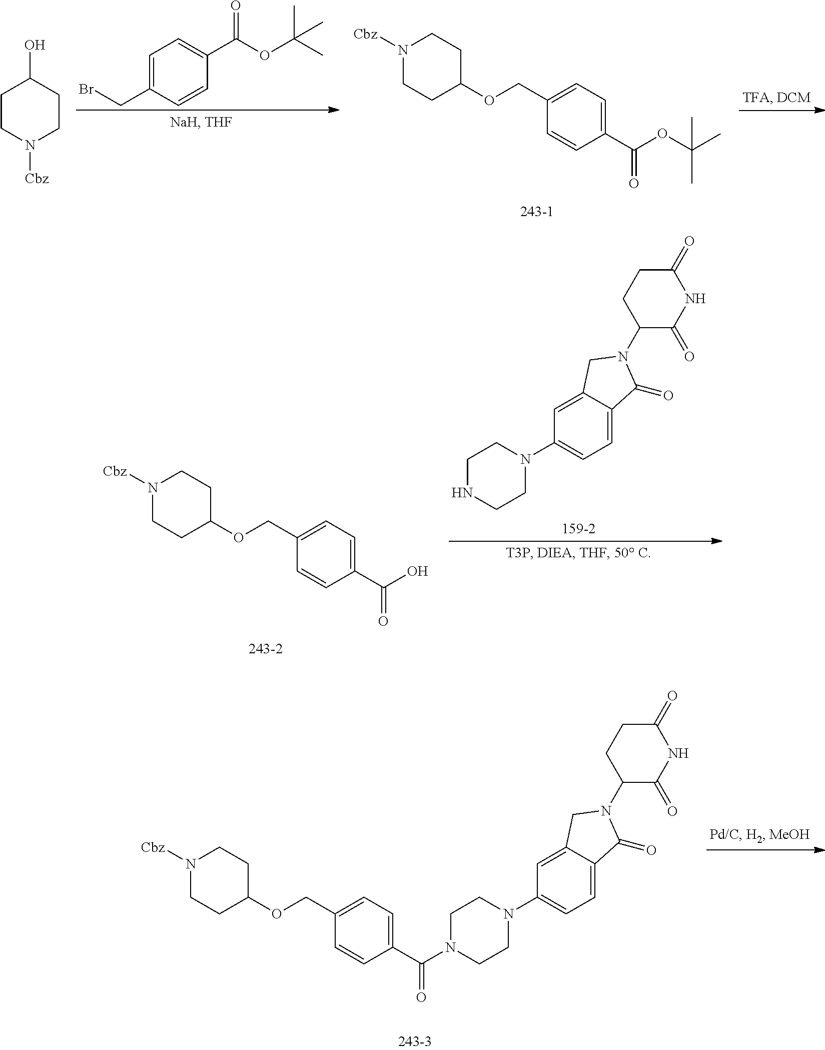

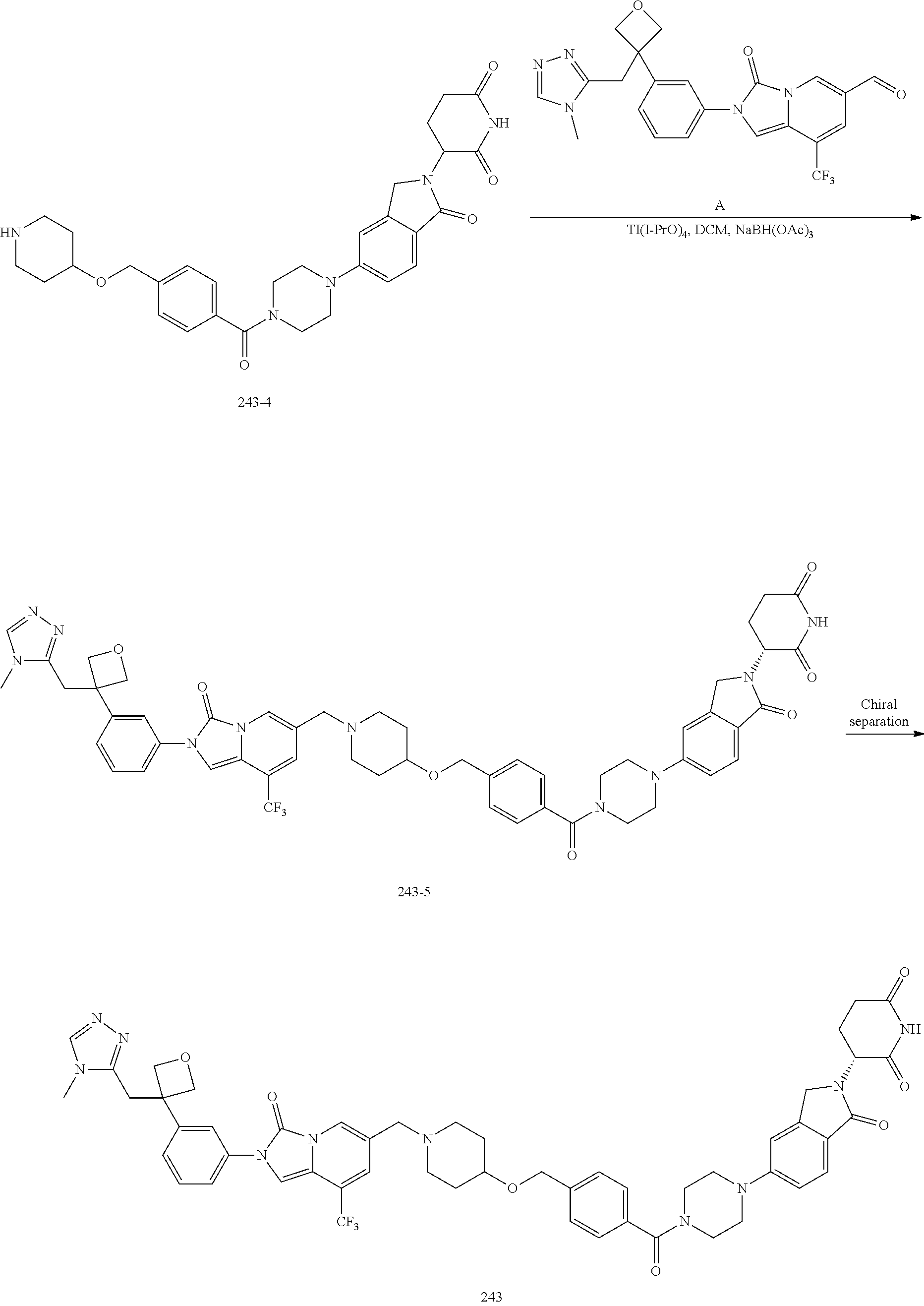

### Synthesis of Compound 243-1.

A solution of benzyl 4-hydroxypiperidine-1-carboxylate (6 g, 25.501 mmol, 1 equiv) in THF (60 mL) was treated with NaH (0.73 g, 30.601 mmol, 1.2 equiv) for 30 min at 0° C. under nitrogen atmosphere followed by the addition of tert-butyl 4-(bromomethyl) benzoate (6.91 g, 25.501 mmol, 1 equiv) at room temperature. The resulting mixture was stirred for overnight at room temperature under nitrogen atmosphere. The reaction was quenched with sat. NH₄Cl (aq) (180 ml) at room temperature. The resulting mixture was concentrated under vacuum. The residue was purified by silica gel column chromatography, eluted with CH₂Cl₂/MeOH (60:1) to afford Compound 243-1 (7.8 g, 68.29%) as a white solid.

### Synthesis of Compound 243-2.

Into a 250 mL 3-necked round-bottom flask were added Compound 243-1 (7.8 g, 18.330 mmol, 1 equiv), TFA (60 mL) and DCM (20 mL) at room temperature. The resulting mixture was stirred for 2 h at room temperature. The mixture was basified to pH 7 with saturated NaHCO₃ (aq.). The aqueous layer was extracted with CH₂Cl₂/MeOH (10:1) (3×50 mL). The resulting mixture was concentrated under vacuum. This resulted in Compound 243-2 (6 g, 84.18%) as a white solid.

### Synthesis of Compound 243-3.

A solution of Compound 243-2 (2 g, 5.414 mmol, 1 equiv) in THF (20 mL) was treated with Compound 159-2 (1.78 g, 5.414 mmol, 1 equiv) and DIEA (1.40 g, 10.828 mmol, 2 equiv) for 30 min at room temperature under nitrogen atmosphere followed by the addition of T₃P (5.17 g, 16.242 mmol, 3 equiv) at room temperature. The resulting mixture was stirred for overnight at 50° C. under nitrogen atmosphere. The reaction was quenched by the addition of sat. NH₄Cl (aq.) (60 mL) at room temperature. The aqueous layer was extracted with CH₂Cl₂ (3×30 mL). The resulting mixture was concentrated under vacuum. The residue was purified by silica gel column chromatography, eluted with PE/EA (1:1) to afford Compound 243-3 (1.6 g, 41.30%) as a brown solid.

### Synthesis of Compound 243-4.

To a solution of Compound 243-3 (1.76 g, 2.589 mmol, 1 equiv) in MeOH (51 mL) was added Pd/C (0.26 g, 2.485 mmol, 0.96 equiv) under nitrogen atmosphere in a 50 mL round-bottom flask. The mixture was hydrogenated at room temperature for 1 h under hydrogen atmosphere using a hydrogen balloon, filtered through a Celite pad and concentrated under reduced pressure. This resulted in Compound 243-4 (1.31 g, 82.53%) as a white solid.

### Synthesis of Compound 243-5.

A solution of Compound 243-4 (478 mg, 0.876 mmol, 1 equiv) in DCM (5 mL) was treated with Intermediate A (400.71 mg, 0.876 mmol, 1 equiv) and Ti(Oi-Pr)₄ (995.94 mg, 3.504 mmol, 4 equiv) for 30 min at room temperature under nitrogen atmosphere followed by the addition of NaBH(OAc)₃ (557.00 mg, 2.628 mmol, 3 equiv) at room temperature. The resulting mixture was stirred for 2 h at room temperature under nitrogen atmosphere. The mixture was acidified to pH 7 with AcOH (10%). The resulting mixture was washed with 1×100 mL of EtOAc. The aqueous layer was extracted with CH₂Cl₂/MeOH (5:1) (5×100 mL). The resulting mixture was concentrated under vacuum. The crude product (500 mg) was purified by Prep-HPLC with the following conditions (Column: Sunfire prep C18 column, 30*150 mm, 5 μm; Mobile Phase A: Water (0.1% FA), Mobile Phase B: ACN; Flow rate: 60 mL/min; Gradient: 15% B to 25% B in 10 min. 25% B; Wave Length: 254; 220 nm; RT1 (min): 9.05; Number Of Runs: 0) to afford Compound 243-5 (191 mg, 20.98%) as a yellow solid.

### Synthesis of Compound 243.

The Compound 243-5 (191 mg) was purified by Prep-CHIRAL-HPLC with the following conditions (Column: CHIRALPAK IH, 2*25 cm, 5 μm; Mobile Phase A: Hex (0.2% TEA)—HPLC, Mobile Phase B: IPA:DCM=1:1—HPLC; Flow rate: 20 mL/min; Gradient: 80% B to 80% B in 13 min; Wave Length: 220/254 nm; RT1 (min): 7.31; RT2 (min): 10.46; Sample Solvent: IPA:DCM=1:1—HPLC; Injection Volume: 0.8 mL; Number of Runs: 1, The first peak was the product) to afford Compound 243 (40.9 mg, 62.80%) as a white solid.

LC-MS-Compound 243: (ES, m/z): [M+H]⁺ 988

H-NMR-Compound 243: (400 MHz, DMSO-d6, ppm): δ 1.53-1.55 (m, 2H), δ1.88-1.90 (m, 3H), δ2.15-2.70 (m, 2H), δ2.32-2.40 (m, 1H), δ2.55-2.65 (m, 1H), δ2.79 (m, 2H), δ2.90-3.05 (m, 5H), δ3.29-3.31 (m, 3H), δ3.50 (s, 4H), δ3.52 (s, 3H), δ3.65-3.75 (s, 2H), δ4.16-4.20 (d, 1H), δ4.29-4.31 (d, 1H), δ4.55 (s, 2H), δ 4.90-4.95 (m, 4H), δ5.02-5.07 (m, 1H), δ6.88-6.90 (d, 1H), δ7.02-7.09 (m, 3H), δ7.31 (s, 1H), δ7.37-7.42 (m, 6H), δ7.53-7.55 (d, 1H), δ7.66 (s, 1H), δ7.73-7.76 (m, 1H), δ8.19 (s, 1H), δ10.95 (s, 1H).

### Example 242. Compound 244

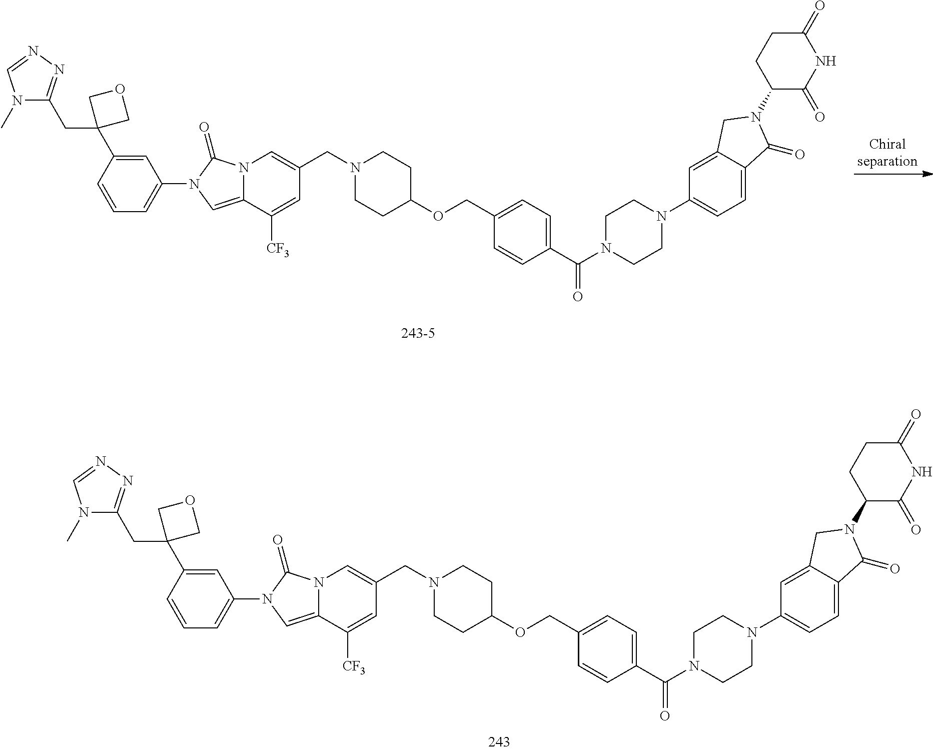 [SMILES: Cn1cnnc1CC1(c2cccc(-n3cc4c(C(F)(F)F)cc(CN5CCC(OCc6ccc(C(=O)N7CCN(c8ccc9c(c8)CN([C@@H]8CCC(=O)NC8=O)C9=O)CC7)cc6)CC5)cn4c3=O)c2)COC1 ; Cn1cnnc1CC1(c2cccc(-n3cc4c(C(F)(F)F)cc(CN5CCC(OCc6ccc(C(=O)N7CCN(c8ccc9c(c8)CN([C@H]8CCC(=O)NC8=O)C9=O)CC7)cc6)CC5)cn4c3=O)c2)COC1]

### Synthesis of Compound 244.

The Compound 243-5 (130 mg) was purified by Prep-CHIRAL-HPLC with the following conditions (Column: CHIRALPAK IH, 2*25 cm, 5 μm; Mobile Phase A: Hex (0.2% TEA)—HPLC, Mobile Phase B: IPA:DCM=1:1—HPLC; Flow rate: 20 mL/min; Gradient: 80% B to 80% B in 13 min; Wave Length: 220/254 nm; RT1 (min): 7.31; RT2 (min): 10.46. Sample Solvent: IPA:DCM=1:1—HPLC; Injection Volume: 0.8 mL; Number of Runs: 1, The second peak was the product) to afford Compound 244 (49 mg, 37.6%) as a yellow solid.

LCMS-Compound 244: (ES, m/z): [M+H]⁺ 988

NMR-Compound 244: (400 MHz, DMSO-d6, ppm): δ 1.53-1.55 (m, 2H), δ1.88-1.90 (m, 3H), δ2.15-2.70 (m, 2H), δ2.32-2.40 (m, 1H), δ2.55-2.65 (m, 1H), δ2.79 (m, 2H), δ2.90-3.05 (m, 5H), δ3.29-3.31 (m, 3H), δ3.50 (s, 4H), δ3.52 (s, 3H), δ3.65-3.75 (s, 2H), δ4.16-4.20 (d, 1H), δ4.29-4.31 (d, 1H), δ4.55 (s, 2H). δ 4.90-4.95 (m, 4H), δ5.02-5.07 (m, 1H), δ6.88-6.90 (d, 1H), δ7.02-7.09 (m, 3H), δ7.31 (s, 1H), δ7.37-7.42 (m, 6H), δ7.53-7.55 (d, 1H), δ7.66 (s, 1H), δ7.73-7.76 (m, 1H), δ8.19 (s, 1H), δ10.95 (s, 1H).

### Example 243. Compound 245

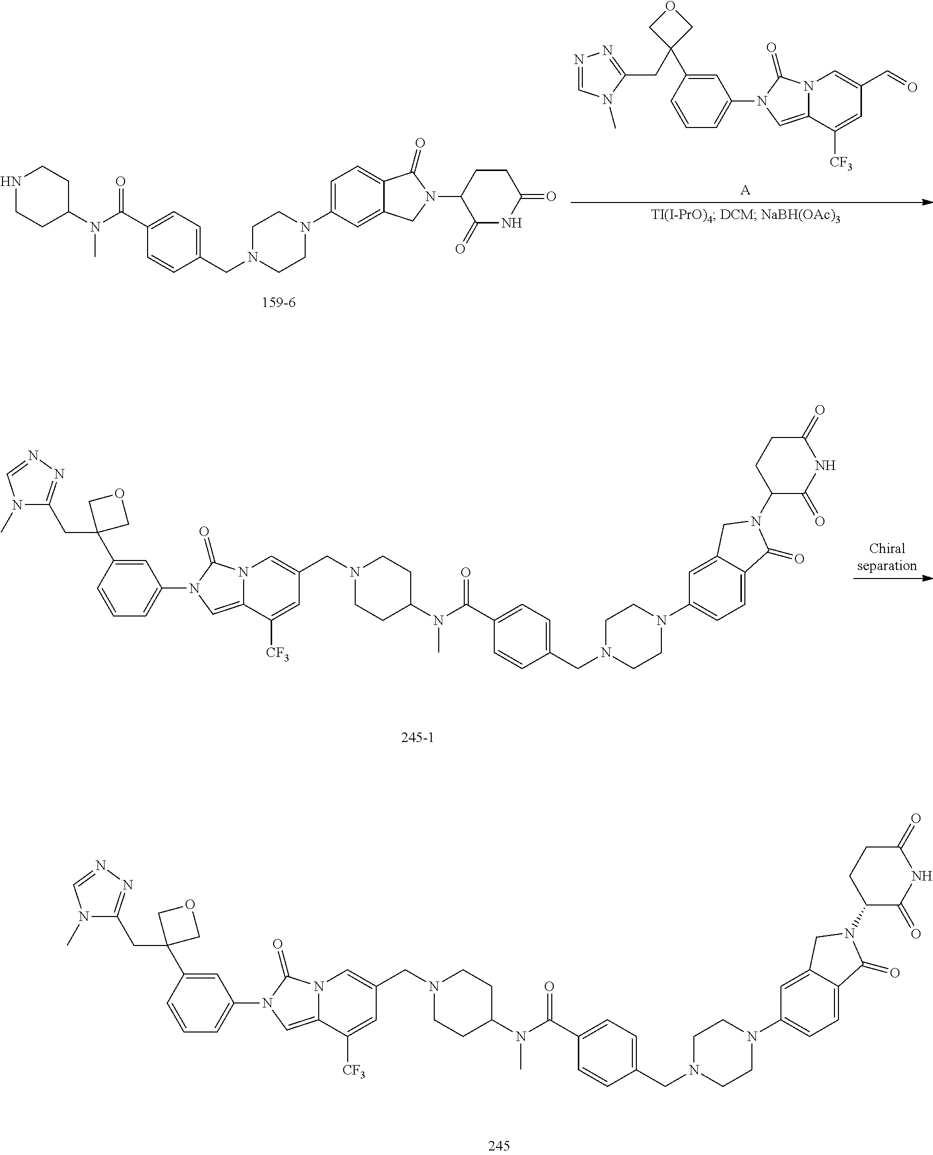

### Synthesis of Compound 245-1.

A solution of Compound 159-6 (800 mg, 1.432 mmol, 1 equiv) and Intermediate A (654.99 mg, 1.432 mmol, 1 equiv) in DCM (16 mL) was treated with Ti(Oi-Pr)₄ (1627.94 mg, 5.728 mmol, 4 equiv) for 1 h at room temperature under nitrogen atmosphere followed by the addition of NaBH(OAc)₃ (910.46 mg, 4.296 mmol, 3 equiv) at room temperature. The resulting mixture was stirred for 5 h at room temperature under nitrogen atmosphere. The reaction was quenched by the addition of Water/HOAc=10/1 (100 mL) at room temperature. The resulting mixture was extracted with CH₂Cl₂/MeOH=5/1 (4×150 mL). The combined organic layers were concentrated under reduced pressure. The residue was purified by Prep-HPLC with the following conditions (Column: Sunfire prep C18 column, 30*150 mm, 5 μm; Mobile Phase A: Water (0.1% FA), Mobile Phase B: ACN; Flow rate: 60 mL/min; Gradient: 7% B to 20% B in 7 min, 20% 13; Wave Length: 254; 220 nm; RT1 (min): 6.75; to afford Compound 245-1 (230 mg, 16.06%) as a yellow solid.

### Synthesis of Compound 245.

Compound 245-1 (230 mg, 0.230 mmol, 1 equiv) was purified by Prep-Chiral-HPLC with the following conditions (Column: CHIRALPAK IH, 2*25 cm, 5 μm, Mobile Phase A: MTBE (0.1% TEA), Mobile Phase B: DCM:ACN=1:1; Flow rate: 20 mL/min; Gradient: 90% B to 90% B in 20 min; Wave Length: 220/254 nm; RT1 (min): 10.29; RT2 (min): 16.04: The first peak was product) to afford Compound 245 (97.7 mg, 42.39%) as a yellow solid.

LCMS-Compound 245: (ES, m/z): [M+H]⁺ 1000

NMR-Compound 245: (400 MHz, DMSO, δ ppm): δ1.51-1.92 (m, 5H), 1.94-1.99 (m, 1H), 2.02-2.23 (m, 1H), 2.31-2.39 (m, 1H), 2.52-2.53 (m, 2H), 2.53-2.60 (m, 4H), 2.79-2.95 (m, 6H), 2.97 (s, 3H), 3.32-3.43 (m, 6H), 3.53-3.65 (m, 4H), 4.18-4.22 (d, 1H), 4.30-4.35 (d, 2H), 4.90-5.02 (m, 4H), 5.03-5.06 (m, 1H), 6.89-6.91 (d, 1H), 7.04-7.07 (m, 3H), 7.32-7.41 (m, 7H), 7.51-7.75 (m, 3H), 8.20 (s, 1H), 10.99 (s, 1H).

### Example 244. Compound 246

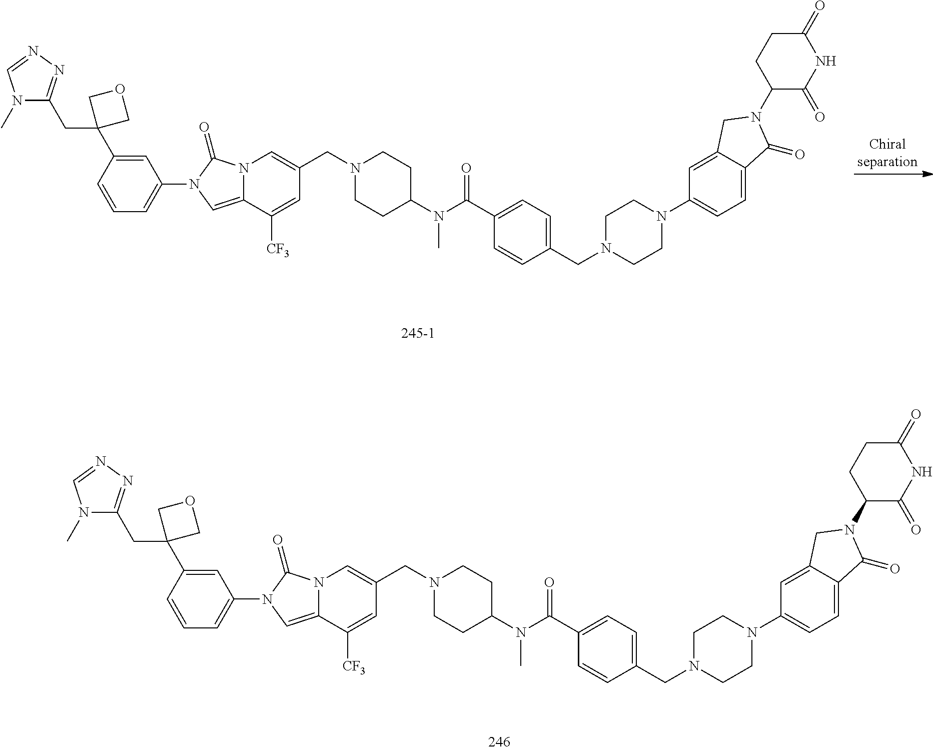 [SMILES: CN(C(=O)c1ccc(CN2CCN(c3ccc4c(c3)CN(C3CCC(=O)NC3=O)C4=O)CC2)cc1)C1CCN(Cc2cc(C(F)(F)F)c3cn(-c4cccc(C5(Cc6nncn6C)COC5)c4)c(=O)n3c2)CC1 ; CN(C(=O)c1ccc(CN2CCN(c3ccc4c(c3)CN([C@H]3CCC(=O)NC3=O)C4=O)CC2)cc1)C1CCN(Cc2cc(C(F)(F)F)c3cn(-c4cccc(C5(Cc6nncn6C)COC5)c4)c(=O)n3c2)CC1]

### Synthesis of Compound 246.

Compound 245-1 (230 mg) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 90% B to 90% B in 20 min; Wave Length: 220/254 nm; RT1 (min): 10.29; RT2 (min): 16.04; the second peak was product) to afford Compound 246 (99.9 mg, 43.43%) as a yellow solid.

LCMS-Compound 246: (ES, m/z): [M+H]⁺ 1000

NMR-Compound 246: (400 MHz, DMSO, δ ppm): δ1.51-1.97 (m, 6H), 2.02-2.23 (m, 1H), 2.31-2.39 (m, 1H), 2.52-2.53 (m, 2H), 2.53-2.60 (m, 4H), 2.79-2.95 (m, 6H), 2.97 (s, 3H), 3.32-3.43 (m, 6H), 3.53-3.65 (m, 4H), 4.18-4.22 (d, 1H), 4.30-4.35 (d, 2H), 4.90-5.02 (m, 4H), 5.03-5.06 (m, 1H), 6.89-6.91 (d, 1H), 7.04-7.07 (m, 3H), 7.32-7.41 (m, 7H), 7.51-7.75 (m, 3H), 8.20 (s, 1H), 10.99 (s, 1H).

### Example 245. Compound 247

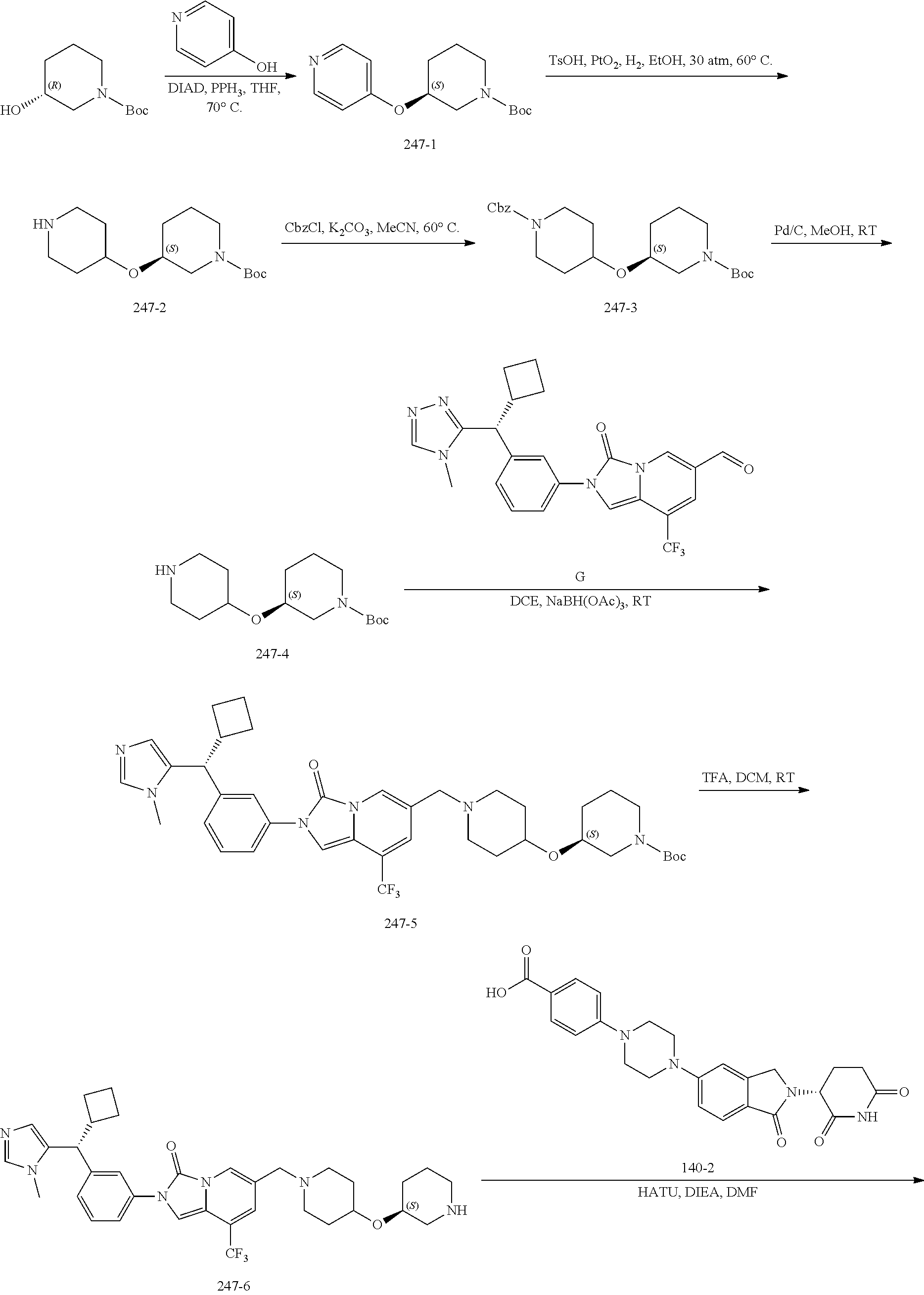

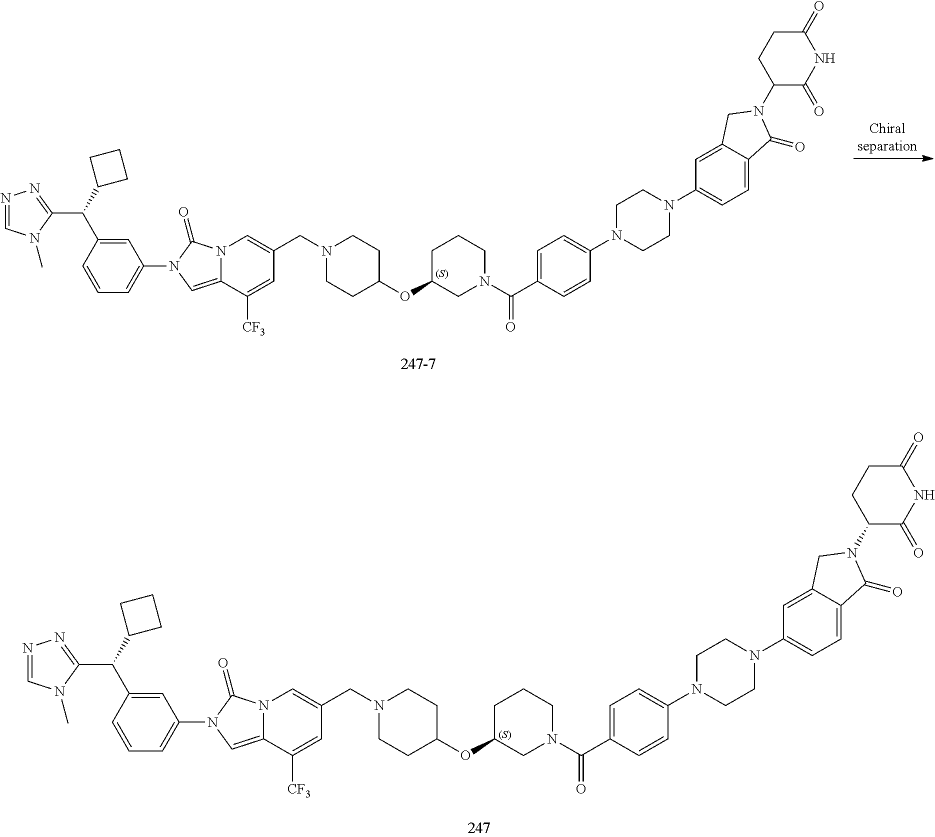

### Synthesis of Compound 247-1.

To a stirred solution of tert-butyl (3R)-3-hydroxypiperidine-1-carboxylate (50 g, 248.427 mmol, 1.5 equiv) and 4-hydroxypyridine (15.75 g, 165.618 mmol, 1 equiv) in THF (500 mL) were added DIAD (66.98 g, 331.236 mmol, 2 equiv) and PPH₃ (86.88 g, 331.236 mmol, 2 equiv) at room temperature under nitrogen atmosphere. The resulting mixture was stirred for overnight at 70° C. under nitrogen atmosphere. The reaction was quenched by the addition of Water (1000 mL) at room temperature. The aqueous layer was extracted with DCM (3×500 mL). The resulting mixture was concentrated under reduced pressure. The residue was purified by silica gel column chromatography, eluted with DCM/MeOH (30:1) to afford Compound 247-1 (10 g, 195.23%) as a white solid.

### Synthesis of Compound 247-2.

To a solution of Compound 247-1 (10 g, 35.926 mmol, 1 equiv) in 250 mL EtOH was added PtO₂ (0.98 g, 4.311 mmol, 0.2 equiv) and TsOH (1.24 g, 7.185 mmol, 0.2 equiv) in a pressure tank. The mixture was hydrogenated at 70° C. under 30 atm of hydrogen pressure for overnight, filtered through a Celite pad and concentrated under reduced pressure. This resulted in Compound 247-2 (10 g, 39.15%) as a brown solid.

### Synthesis of Compound 247-3.

To a stirred solution of Compound 247-2 (10 g, 35.162 mmol, 1 equiv) and K₂CO₃ (9.72 g, 70.324 mmol, 2 equiv) in MeCN (100 mL) were added CbzCl (12.00 g, 70.324 mmol, 2 equiv) at room temperature. The resulting mixture was stirred for overnight at 60° C. The reaction was quenched by the addition of Water (120 mL) at room temperature. The aqueous layer was extracted with DCM (3×80 mL). The resulting mixture was concentrated under reduced pressure. The residue was purified by silica gel column chromatography, eluted with PE/EA (5:1) to afford Compound 247-3 (1.6 g, 9.79%) as a brown solid.

### Synthesis of Compound 247-4.

To a solution of Compound 247-3 (1.6 g, 3.823 mmol, 1 equiv) in 20 mL MeOH was added Pd/C (10%, 160 mg) under nitrogen atmosphere. The mixture was hydrogenated at room temperature for overnight under hydrogen atmosphere using a hydrogen balloon, filtered through a Celite pad and concentrated under reduced pressure. This resulted in Compound 247-4 (1.2 g, 99.34%) as a brown solid.

### Synthesis of Compound 247-5.

To a stirred solution of Compound 247-4 (1.15 g, 4.044 mmol, 1 equiv) and intermediate G (1.84 g, 4.044 mmol, 1 equiv) in DCE (20 ml) were added NaBH(OAc)₃ (1.71 g, 8.088 mmol, 2 equiv) at room temperature. The resulting mixture was stirred for overnight at room temperature. The reaction was quenched with Water (60 mL) at room temperature. The aqueous layer was extracted with DCM (3×15 mL). The resulting mixture was concentrated under reduced pressure. The residue was purified by silica gel column chromatography, eluted with DCM/MeOH (30:1) to afford Compound 247-5 (1.2 g, 34.85%) as a yellow solid.

### Synthesis of Compound 247-6.

To a stirred solution of Compound 247-5 (1.2 g, 1.658 mmol, 1 equiv) in DCM (15 mL) was added TFA (2 mL) at room temperature. The resulting mixture was stirred for 4 h at room temperature. The resulting mixture was concentrated under reduced pressure. The residue was purified by reversed-phase flash chromatography with the following conditions: column, C18 silica gel; mobile phase, MeCN in Water (0.1% FA), 30% to 45% gradient in 20 min; detector, UV 254 nm. This resulted in Compound 247-6 (1 g, 82.20%) as a yellow solid.

### Synthesis of Compound 247-7.

To a stirred solution of Compound 140-2 (0.72 g, 1.603 mmol, 1 equiv) and DIEA (0.52 g, 4.008 mmol, 2.5 equiv) in DMF (100 mL) was added HATU (1.52 g, 4.008 mmol, 2.5 equiv) at room temperature. The resulting mixture was stirred for 1 h at room temperature. To the above mixture was added Compound 247-6 (1 g, 1.603 mmol, 1 equiv) at room temperature. The resulting mixture was stirred for additional overnight at room temperature. The reaction was quenched with Water (100 mL) at room temperature. The aqueous layer was extracted with EtOAc (3×50 mL). The resulting mixture was concentrated under reduced pressure. The crude product (600 mg) was purified by Prep-HPLC with the following conditions (Column: Sunfire prep C18 column, 30*150 mm, 5 μm; Mobile Phase A: Water (0.1% FA), Mobile Phase B: ACN; Flow rate: 60 mL/min; Gradient: 22% B to 33% B in 7 min, 33% B: Wave Length: 254; 220 nm; RT1 (min); 6.98; to afford Compound 247-7 (200 mg, 11.24%) as a yellow solid.

### Synthesis of Compound 247.

The Compound 247-7 (200 mg, 11.24%) was purified by Chiral Separation with the following conditions (Column: CHIRALPAK IH, 2*25 cm, 5 μm, Mobile Phase A: MTBE (0.1% TEA)—HPLC, Mobile Phase B: DCM:ACN=1:1—HPLC; Flow rate: 20 mL/min; Gradient: 90% B to 90% B in 16 min; Wave Length: 220/254 nm; RT1 (min): 7.49; RT2 (min): 12.97; the first peak was product) to afford crude product (70 mg). The crude product (70 mg) was purified by Prep-HPLC with the following conditions: Column: (Xselect CSH C18 OBD Column 30*150 mm 5 μm, n; Mobile Phase A: Water (0.1% FA), Mobile Phase B: ACN; Flow rate: 60 mL/min; Gradient: 23% B to 34% B in 7 min, 34% B; Wave Length: 254; 220 nm; RT1 (min): 6.65) to afford Compound 247 (45.1 mg, 22.14%) as a yellow solid.

LC-MS-Compound 247: (ES, m/z): [M+H]⁺ 1054

H-NMR-Compound 247: (400 MHz, DMSO-d6, δ ppm): 1.21-1.59 (m, 4H), 1.69-1.79 (m, 9H), 1.96-2.07 (m, 1H), 2.07-2.09 (m, 3H), 2.48-2.49 (m, 1H), 2.50-2.66 (m, 2H), 2.67-2.95 (m, 2H), 3.25-3.29 (m, 3H), 3.32-3.39 (m, 10H), 3.40-3.45 (m, 7H), 4.20-4.36 (m, 3H), 5.04-5.07 (m, 1H), 7.00-7.02 (m, 3H), 7.11-7.13 (m, 2H), 7.17-7.19 (m, 1H), 7.30-7.31 (d, 3H), 7.41-7.43 (d, 1H), 7.45-7.53 (m, 1H), 7.64-7.73 (m, 3H), 8.31 (s, 1H), 10.90 (s, 1H).

### Example 246. Compound 248

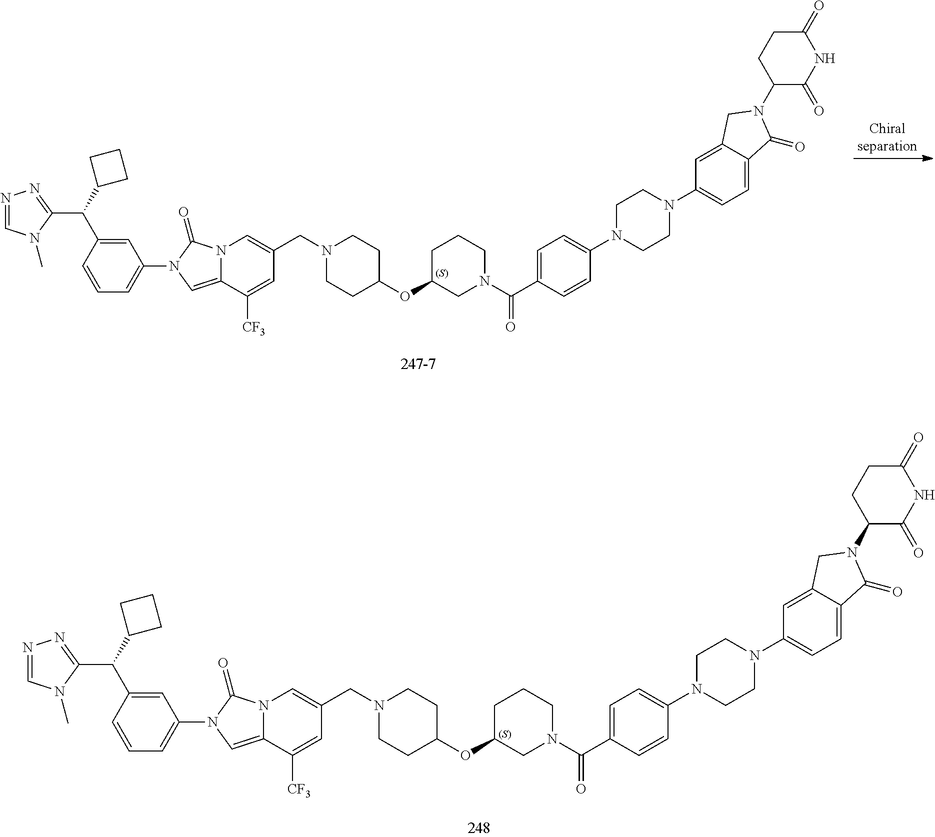

### Synthesis of Compound 248.

The Compound 247-7 (200 mg, 11.24%) was purified by Chiral Separation with the following conditions (Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 90% B to 90% B in 16 min; Wave Length: 220/254 nm; RT1 (min): 7.49; RT2 (min): 12.97; the second peak was product) to afford crude product (80 mg). The crude product (80 mg) was purified by Chiral Separation with the following conditions: (Column: CHIRALPAK IH, 2*25 cm, 5 μm; Mobile Phase A: MTBE (0.10% TEA)—HPLC, Mobile Phase B: DCM:ACN=1:1—HPLC; Flow rate: 20 mL/min; Gradient: 90% B to 90% B in 15 min; Wave Length: 220/254 nm; RT1 (min): 5.65; RT2 (min): 7.52) to afford crude product (60 mg). The crude product (60 mg) was purified by reversed-phase flash chromatography with the following conditions: column, 40 g C18 silica gel; mobile phase, MeCN in Water (0.1% FA), 25% to 55% gradient in 8 min; detector, UV 254 nm to afford Compound 248 (43.2 mg, 21.08%) as a yellow solid.

LC-MS-Compound 248: (ES, m/z): [M+H]⁺ 1054

H-NMR-Compound 248: (400 MHz, DMSO-d6, δ ppm): 1.21-1.59 (m, 4H), 1.69-1.79 (m, 9H), 1.96-1.97 (m, 1H), 2.07-2.09 (m, 3H), 2.35-2.37 (m, 1H), 2.50-2.67 (m, 2H), 2.80-2.97 (m, 2H), 3.25-3.32 (m, 3H), 3.32-3.38 (m, 6H), 3.40-3.42 (m, 4H), 3.44-3.45 (m, 7H), 4.19-4.36 (m, 3H), 5.03-5.07 (m, 1H), 7.00-7.02 (m, 3H), 7.11-7.13 (m, 2H), 7.17-7.19 (m, 1H), 7.30-7.31 (d, 3H), 7.41-7.43 (d, 1H), 7.45-7.53 (m, 1H), 7.64-7.73 (m, 3H), 8.31 (s, 1H), 10.90 (s, 1H).

### Example 247. Compound 249

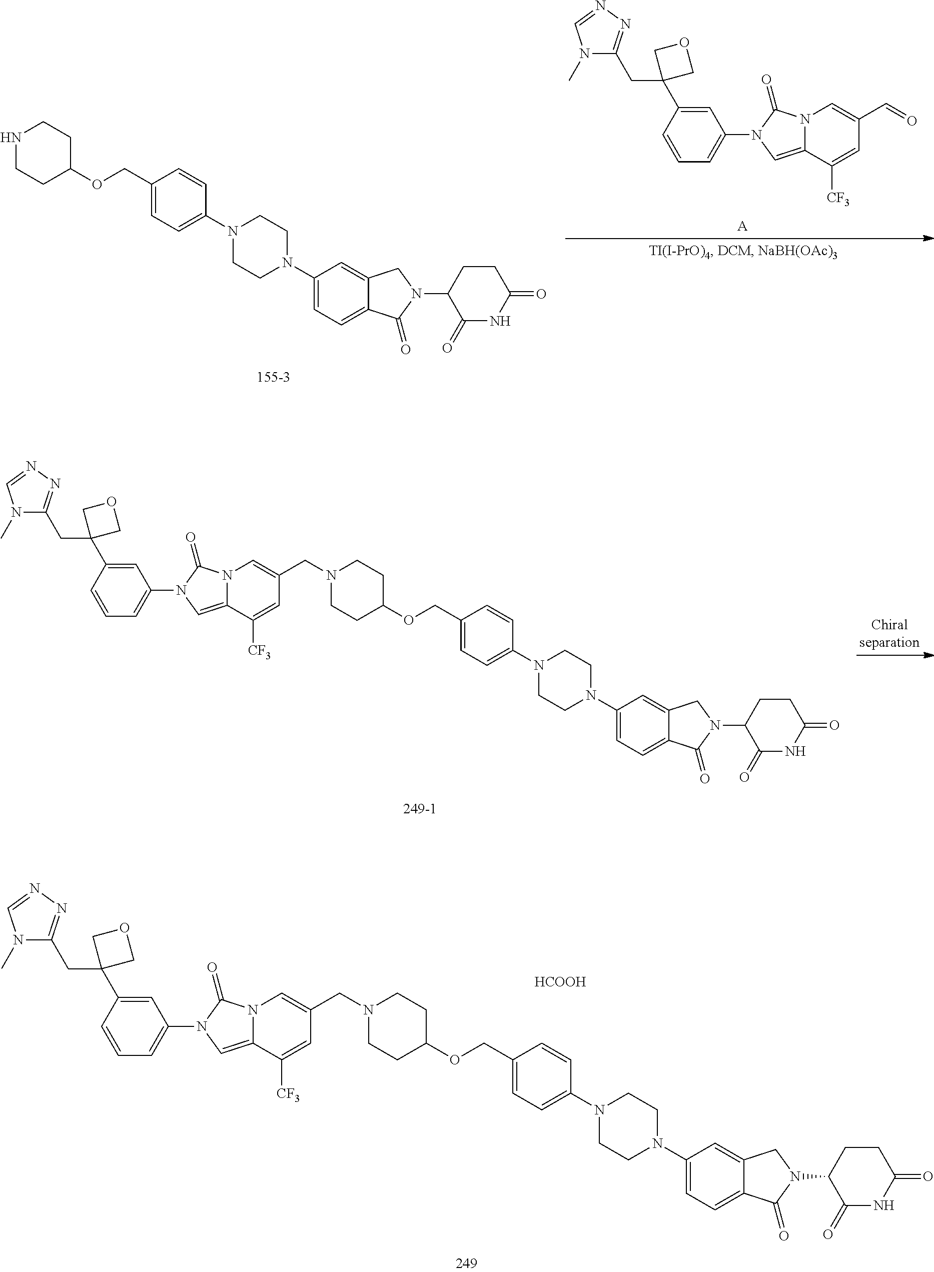

### Synthesis of Compound 249-1.

To a stirred solution of Compound 155-3 (900 mg, 1.73 mmol, 1.0 equiv) and Intermediate A (954 mg, 2.08 mmol, 1.2 equiv) in DCM (9 mL) was added Ti(Oi-Pr)₄ (2 g, 6.96 mmol, 4.0 equiv) at room temperature. The resulting mixture was stirred for overnight at room temperature. To the above mixture was added NaBH(OAc)₃ (1.1 g, 5.21 mmol, 3.0 equiv) at room temperature. The resulting mixture was stirred for additional 2 h at room temperature. The reaction was quenched by the addition of 10% AcOH (100 mL) at room temperature. The aqueous layer was extracted with CH₂Cl₂:MeOH (5:1) (3×100 mL). The residue was purified by reversed-phase flash chromatography with the following conditions: column, C18 silica gel; mobile phase, MeCN in water (0.1% FA), 10% to 50% gradient in 30 min; detector, UV 254 nm. The crude product (400 mg) was purified by Prep-HPLC with the following conditions (Column: Sunfire prep C18 column, 30*150 mm, 5 μm; Mobile Phase A: Water (0.1% FA), Mobile Phase B: ACN; Flow rate: 60 mL/min; Gradient: 18% B to 30% B in 7 min, 30% B: Wave Length: 254; 220 nm; RT1 (min): 6.5; Number of Runs: 0) to afford Compound 249-1 (190 mg, 11%) as a yellow solid.

### Synthesis of Compound 249.

The Compound 249-1 (190 mg) was purified by Prep-CHIRAL-HPLC with the following conditions Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 80% B to 80% B in 13 min; Wave Length: 220/254 nm; RT1 (min): 5.93; RT2 (min): 8.44; Sample Solvent: DCM:ACN=1:1—HPLC; Injection Volume: 1.1 mL; Number Of Runs: 4. The first peak was the product. The residue was purified by reversed-phase flash chromatography with the following conditions: column, C18 silica gel; mobile phase, MeCN in water (0.1% FA), 10% to 50% gradient in 30 min; detector, UV 254 nm. This resulted in Compound 249 (49 mg, 25%) as a yellow solid.

LC-MS-Compound 249: (ES, m/z): [M-COOH]⁺ 959

H-NMR-Compound 249: (400 MHz, DMSO-d6, δ ppm): 1.45-1.50 (m, 2H), 1.83-1.85 (m, 2H), 1.95-7.98 (m, 1H), 2.10-2.14 (m, 2H), 2.25-2.35 (m, 1H), 2.49-2.51 (m, 2H), 2.56-2.60 (m, 1H), 2.90-2.96 (m, 4H), 3.27-3.29 (m, 7H), 3.38-3.42 (m, 4H), 3.44-3.52 (m, 2H), 4.20-4.39 (m, 4H), 4.90-4.95 (m, 4H), 5.03-5.06 (m, 1H), 6.87-6.89 (d, 1H), 6.96-7.01 (m, 3H), 7.12-7.14 (m, 2H), 7.18-7.21 (m, 2H), 7.30 (s, 1H), 7.37-7.41 (m, 2H), 7.54-7.56 (s, 1H), 7.66 (s, 1H), 7.73-7.76 (m, 1H), 8.15 (s, 1H), 8.19 (s, 1H), 10.95 (s, 1H).

### Example 248. Compound 250

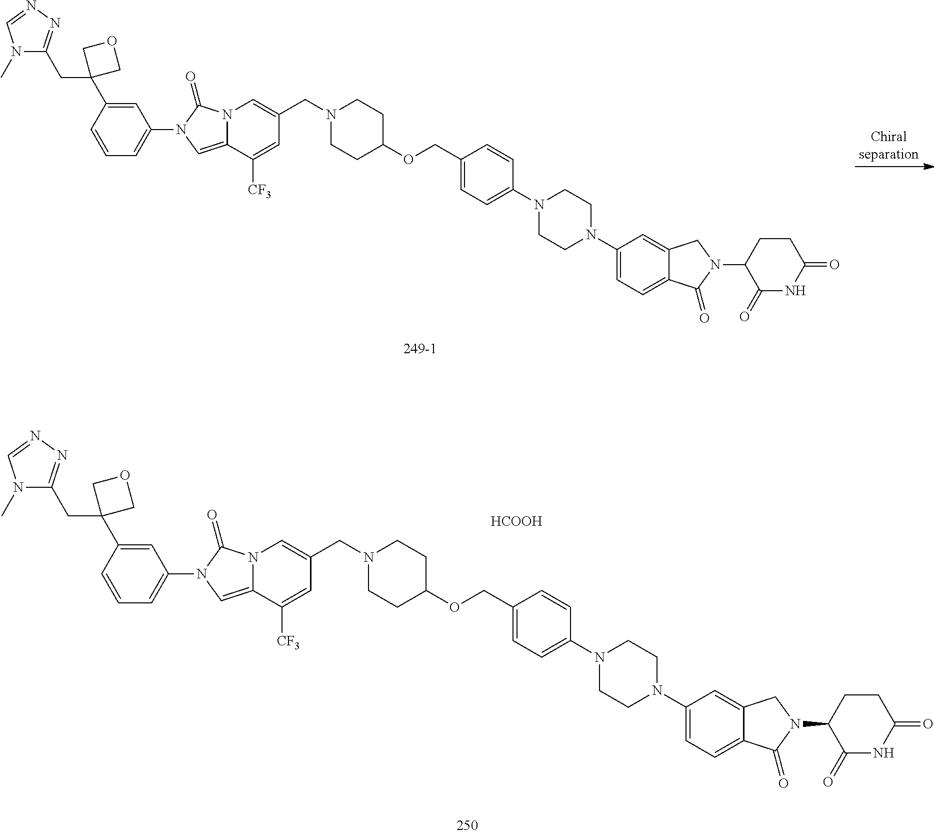 [SMILES: Cn1cnnc1CC1(c2cccc(-n3cc4c(C(F)(F)F)cc(CN5CCC(OCc6ccc(N7CCN(c8ccc9c(c8)CN(C8CCC(=O)NC8=O)C9=O)CC7)cc6)CC5)cn4c3=O)c2)COC1 ; Cn1cnnc1CC1(c2cccc(-n3cc4c(C(F)(F)F)cc(CN5CCC(OCc6ccc(N7CCN(c8ccc9c(c8)CN([C@H]8CCC(=O)NC8=O)C9=O)CC7)cc6)CC5)cn4c3=O)c2)COC1 ; "HCOOH"]

### Synthesis of Compound 250.

The Compound 249-1 (190 mg) was purified by Prep-CHIRAL-HPLC with the following conditions Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 80% B to 80% B in 13 min; Wave Length: 220/254 nm; RT1 (min): 5.93; RT2 (min): 8.44; Sample Solvent: DCM:ACN=1:1—HPLC; Injection Volume: 1.1 mL; Number Of Runs: 4. The second peak was the product. The residue was purified by reversed-phase flash chromatography with the following conditions: column, C18 silica gel; mobile phase, MeCN in water (0.1% FA), 10% to 50% gradient in 30 min; detector, UV 254 nm. This resulted in Compound 250 (56 mg, 27%) as a yellow solid.

LC-MS-Compound 250: (ES, m/z): [M-COOH]⁺ 959

H-NMR-Compound 250: (400 MHz, DMSO-d6, δ ppm): 1.45-1.50 (m, 2H), 1.83-1.85 (m, 2H), 1.95-7.98 (m, 1H), 2.10-2.14 (m, 2H), 2.25-2.35 (m, 1H), 2.49-2.51 (m, 2H), 2.56-2.60 (m, 1H), 2.90-2.96 (m, 4H), 3.27-3.29 (m, 7H), 3.38-3.42 (m, 4H), 3.44-3.52 (m, 2H), 4.20-4.39 (m, 4H), 4.90-4.95 (m, 4H), 5.03-5.06 (m, 1H), 6.87-6.89 (d, 1H), 6.96-7.01 (m, 3H), 7.12-7.14 (m, 2H), 7.18-7.21 (m, 2H), 7.30 (s, 1H), 7.37-7.41 (m, 2H), 7.54-7.56 (s, 1H), 7.66 (s, 1H), 7.73-7.76 (m, 1H), 8.15 (s, 1H), 8.19 (s, 1H), 10.95 (s, 1H).

### Example 249. Compound 251

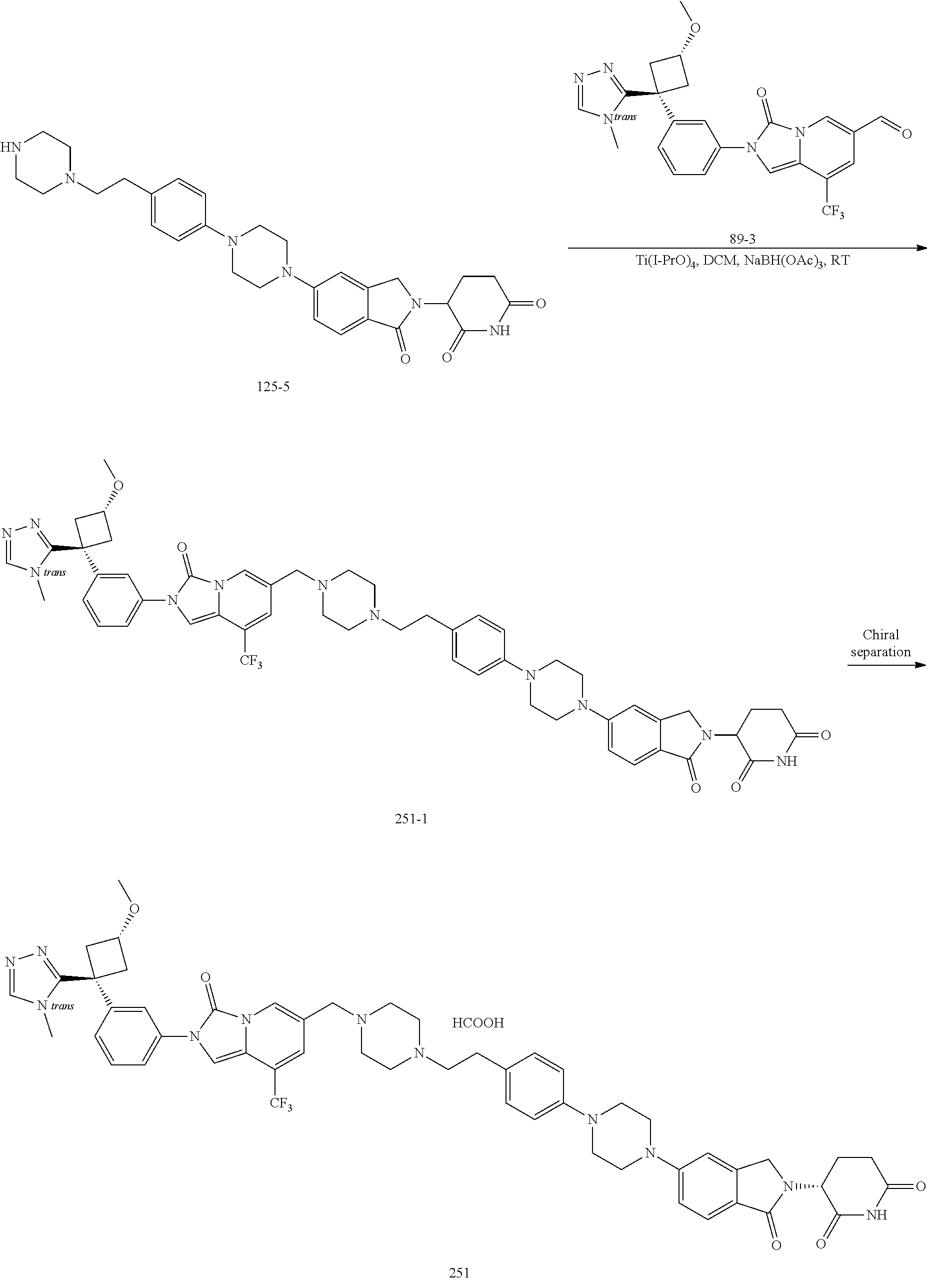 [SMILES: O=C1CCC(N2Cc3cc(N4CCN(c5ccc(CCN6CCNCC6)cc5)CC4)ccc3C2=O)C(=O)N1 ; CO[C@H]1C[C@](c2cccc(-n3cc4c(C(F)(F)F)cc(C=O)cn4c3=O)c2)(c2nncn2C)C1 ; "89-3" ; CO[C@H]1C[C@](c2cccc(-n3cc4c(C(F)(F)F)cc(CN5CCN(CCc6ccc(N7CCN(c8ccc9c(c8)CN(C8CCC(=O)NC8=O)C9=O)CC7)cc6)CC5)cn4c3=O)c2)(c2nncn2C)C1 ; CO[C@H]1C[C@](c2cccc(-n3cc4c(C(F)(F)F)cc(CN5CCN(CCc6ccc(N7CCN(c8ccc9c(c8)CN([C@@H]8CCC(=O)NC8=O)C9=O)CC7)cc6)CC5)cn4c3=O)c2)(c2nncn2C)C1 ; "HCOOH"]

### Synthesis of Compound 251-1.

To a stirred solution of Compound 125-5 (1 g, 1.936 mmol, 1 equiv) and Compound 69-3 (1.10 g, 2.323 mmol, 1.2 equiv) in DCM (15 mL) were added Ti(Oi-Pr)₄ (1.65 g, 5.808 mmol, 3 equiv) at room temperature. The resulting mixture was stirred for overnight at room temperature. To the above mixture was added STAB (0.82 g, 3.872 mmol, 2 equiv) at room temperature. The resulting mixture was stirred for 2 h at room temperature. The mixture was acidified to pH 5 with HOAc (1.5 mi). The resulting mixture was concentrated under reduced pressure. The crude product (700 mg) was purified by Prep-HPLC with the following conditions (Column: Sunfire prep C18 column, 30*150 mm, 5 μm; Mobile Phase A: Water (0.1% FA), Mobile Phase B: ACN; Flow rate: 60 mL/min; Gradient: 22% B to 33% B in 7 min, 33% B; Wave Length: 254; 220 nm; RT1 (min): 6.1) to afford Compound 251-1 (450 mg, 22.96%) as a yellow solid.

### Synthesis of Compound 251.

The Compound 251-1 (450 mg) was purified by Prep-Chiral-HPLC with the following conditions (Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 70% B to 70% B in 10.5 min; Wave Length: 220/254 nm; RT1 (min): 6.54; RT2 (min): 9.18; the first product was the product. Sample Solvent: DCM:ACN=1:1—HPLC; Injection Volume: 0.5 mL; Number of Runs: 15) to afford the crude product. The crude product was purified by reversed-phase flash chromatography with the following conditions: column, 40 g C18 silica gel; mobile phase, MeCN in water (0.1% FA), 30% to 60% gradient in 9 min; detector, UV 254 nm to afford Compound 251 (107.1 mg, 21.95%) as a yellow solid.

LC-MS-Compound 251: (ES, m/z): [M−HCOOH+H]⁺ 972

H-NMR-Compound 251: (400 MHz, DMSO-d6, ppm): δ 1.90-1.98 (m, 1H), δ 2.25-2.50 (m, 7H), δ 2.53-2.69 (m, 9H), δ 2.86-2.98 (m, 1H), δ 3.13 (s, 3H), δ 3.16-3.23 (m, 6H), δ 3.35-3.45 (m, 5H), δ 3.47-3.51 (m, 4H), δ 3.81-3.86 (m, 1H), δ 4.21-4.23 (d, 1H), δ 4.32-4.35 (d, 1H), δ 5.02-5.10 (m, 1H), δ 6.86-6.88 (d, 1H), δ 7.00 (s, 1H), δ 7.14-7.23 (m, 5H), δ 7.40 (s, 1H), δ 7.45-7.47 (d, 1H), δ 7.51 (s, 1H), δ 7.64-7.69 (m, 3H), δ 8.40 (s, 1H), δ 10.96 (s, 1H).

### Example 250. Compound 252

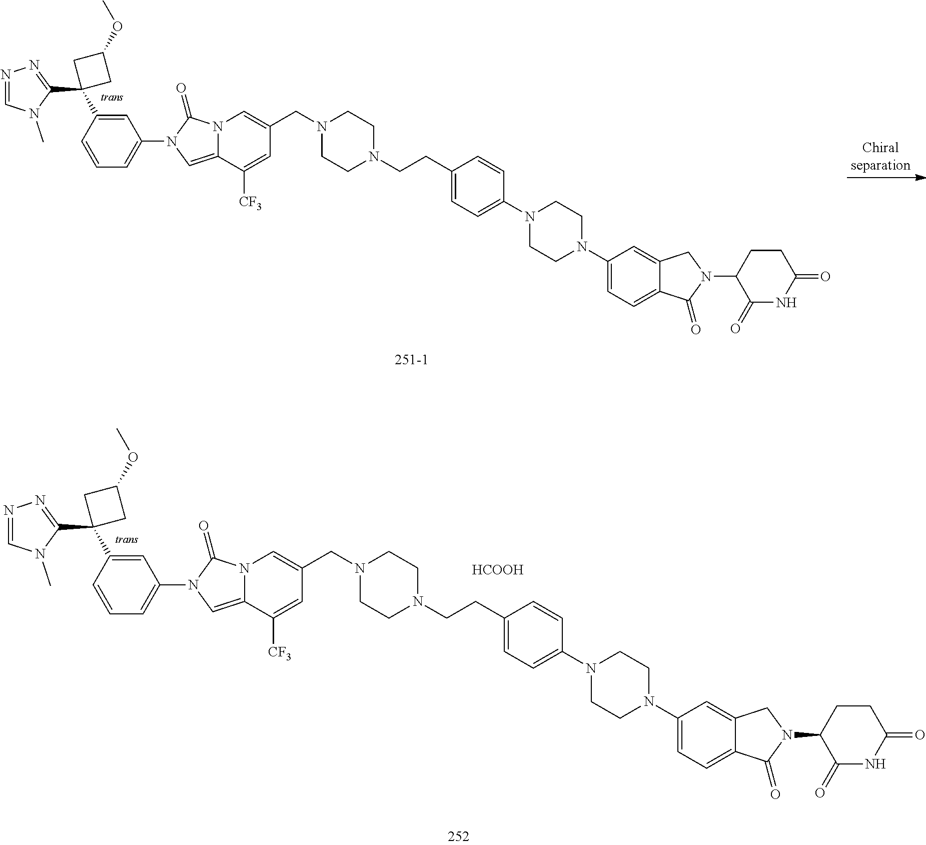 [SMILES: CO[C@H]1C[C@](c2cccc(-n3cc4c(C(F)(F)F)cc(CN5CCN(CCc6ccc(N7CCN(c8ccc9c(c8)CN(C8CCC(=O)NC8=O)C9=O)CC7)cc6)CC5)cn4c3=O)c2)(c2nncn2C)C1 ; CO[C@H]1C[C@](c2cccc(-n3cc4c(C(F)(F)F)cc(CN5CCN(CCc6ccc(N7CCN(c8ccc9c(c8)CN([C@H]8CCC(=O)NC8=O)C9=O)CC7)cc6)CC5)cn4c3=O)c2)(c2nncn2C)C1 ; "HCOOH"]

### Synthesis of Compound 252.

The Compound 251-1 (450 mg) was purified by Prep-Chiral-HPLC with the following conditions (Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 70% B to 70% B in 10.5 min; Wave Length: 220/254 nm; RT1 (min): 6.54; RT2 (min): 9.18; the second product was the product. Sample Solvent: DCM:ACN=1:1—HPLC; Injection Volume: 0.5 mL; Number of Runs: 15) to afford the crude product. The crude product was purified by reversed-phase flash chromatography with the following conditions: column, 40 g C18 silica gel; mobile phase, MeCN in water (0.1% FA), 25% to 60% gradient in 9 min; detector, UV 254 nm to afford Compound 252 (121.7 mg, 25.56%) as a yellow solid.

LC-MS-Compound 252: (ES, m/z): [M−HCOOH+H]⁺ 972

H-NMR-Compound 252: (400 MHz, DMSO-d6, ppm): δ 1.90-1.98 (m, 1H), δ 2.25-2.50 (m, 9H), δ 2.53-2.69 (m, 4H), δ 2.62-2.69 (m, 3H), δ 2.86-2.98 (m, 1H), δ 3.13 (s, 3H), δ 3.16-3.23 (m, 6H), δ 3.35-3.45 (m, 4H), δ 3.47-3.51 (m, 5H), δ 3.81-3.86 (m, 1H), δ 4.21-4.23 (d, 1H), δ 4.32-4.35 (d, 1H), δ 5.02-5.10 (m, 1H), δ 6.86-7.01 (m, 3H), δ 7.14-7.23 (m, 5H), δ 7.40-7.62 (m, 3H), δ 7.64-7.69 (m, 3H), δ 8.40 (s, 1H), δ 10.96 (s, 1H).

### Example 251. Compound 253

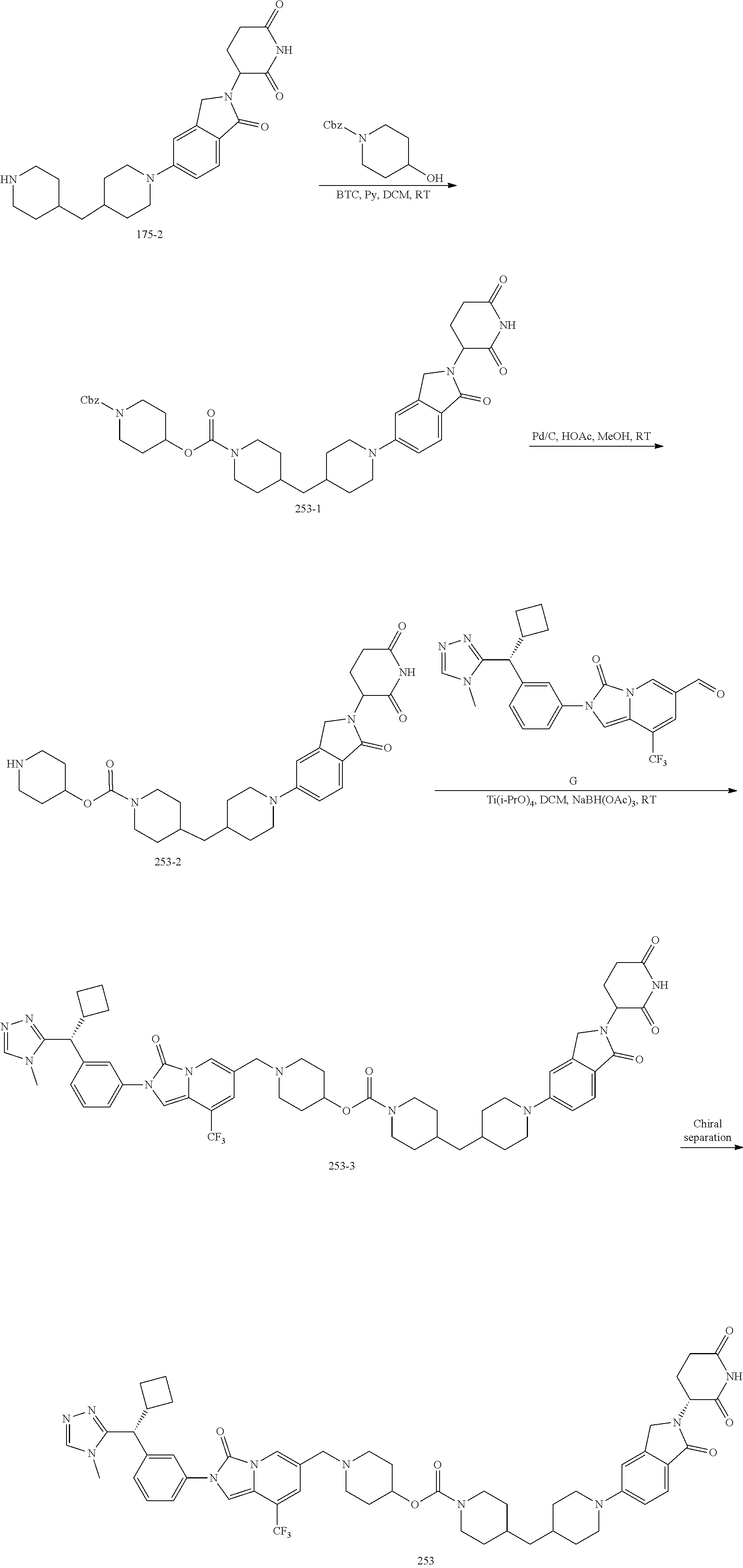

### Synthesis of Compound 253-1.

To a stirred solution of 4-hydroxypiperidine-1-carboxylate (498.78 mg, 2.120 mmol, 1 equiv) and pyridine (1006.11 mg, 12.720 nmol, 6 equiv) in DCM (10 mL) was added BTC (251.62 mg, 0.848 mmol, 0.4 equiv) at room temperature. The resulting mixture was stirred for 30 min at room temperature. To the above mixture was added Compound 175-2 (900 mg, 2.120 mmol, 1 equiv) at room temperature. The resulting mixture was stirred for additional 30 min at room temperature. The resulting mixture was concentrated under vacuum. The residue was purified by reversed-phase flash chromatography with the following conditions: column. C18 silica gel; mobile phase, MeCN in water (0.1% FA), 10% to 50% gradient in 25 min; detector, UV 254 nm. This resulted in Compound 253-3 (550 mg, 37.83%) as a white solid.

### Synthesis of Compound 253-2.

To a solution of Compound 253-1 (500 mg, 0.729 mmol, 1 equiv) in MeOH (20 mL) was added Pd/C (10%, 50 mg) under nitrogen atmosphere in a 50 mL round-bottom flask. The mixture was hydrogenated at room temperature for 1 h under hydrogen atmosphere using a hydrogen balloon, filtered through a Celite pad and concentrated under reduced pressure. This resulted in Compound 253-2 (420 mg, 96.07%) as a yellow solid.

### Synthesis of Compound 253-3.

To a stirred solution of Compound 253-2 (420 mg, 0.761 mmol, 1 equiv) and Intermediate G (485.42 mg, 1.065 mmol, 1.4 equiv) in DCM (10 mL) was added Ti(i-PrO)₄ (432.75 mg, 1.522 mmol, 2 equiv) at room temperature. The resulting mixture was stirred overnight at room temperature. To the above mixture was added NaBH(OAc)₃ (322.70 mg, 1.522 mmol, 2 equiv) at room temperature. The resulting mixture was stirred for additional 4 h at room temperature. The reaction was quenched with 10% AcOH(aq.) (200 mL) at room temperature. The resulting mixture was filtered, the filter cake was washed with 10% AcOH (aq.) (20 mL). The aqueous layer was extracted with EtOAc (100 mL). The aqueous layer concentrated under reduced pressure. The crude product was purified by Prep-HPLC with the following conditions (Column: Sunfire prep C18 column, 30*150 mm, 5 μm; Mobile Phase A: water (0.1% FA), Mobile Phase B: ACN; Flow rate: 60 mL/min; Gradient: 24% B to 34% B in 8 min, Wave Length: 254; 220 nm; RT1 (min): 7.58) to afford Compound 253-2 (100 mg, 13.25%) as a yellow solid.

### Synthesis of Compound 253.

The Compound 253-3 (100 mg) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 80% B to 80% B in 9 min; Wave Length: 220/254 nm; RT1 (min): 4.92; RT2 (min): 7.08, the first peak is product) to afford the crude product. The crude product was purified by Prep-HPLC with the following conditions (Column: Xselect CSH C18 OBD Column 30*150 mm 5 μm, n; Mobile Phase A: water (0.1% FA), Mobile Phase B: ACN; Flow rate: 60 mL/min; Gradient: 25% B to 35% B in 7 min, Wave Length: 254; 220 nm; RT1 (min): 6.43) to afford Compound 259 (22.8 mg, 22.8%) as a yellow solid.

LC-MS-Compound 253: (ES, m/z): [M+H]⁺ 991

H-NMR-Compound 253: (400 MHz, DMSO-d₆, δ ppm): 0.93.99 (m, 2H), 1.11-1.16 (m, 4H), 1.56-1.82 (m, 15H), 1.90-1.99 (m, 1H), 2.03-2.13 (m, 1H), 2.31-2.34 (m, 3H), 2.55-2.60 (m, 3H), 2.76-2.79 (m, 4H), 2.80-2.81 (m, 1H), 3.24-3.27 (m, 3H), 3.43 (s, 3H), 3.83-3.86 (m, 2H), 3.95-3.97 (m, 2H), 4.16-4.33 (m, 3H), 4.59-4.65 (m, 1H), 5.01-5.04 (d, 1H), 7.01-7.03 (m, 3H), 7.17-7.19 (d, 1H), 7.32 (s, 1H), 7.41-7.50 (m, 2H), 7.67-7.73 (m, 3H), 8.32 (s, 1H), 10.95 (s, 1H).

### Example 252. Compound 254

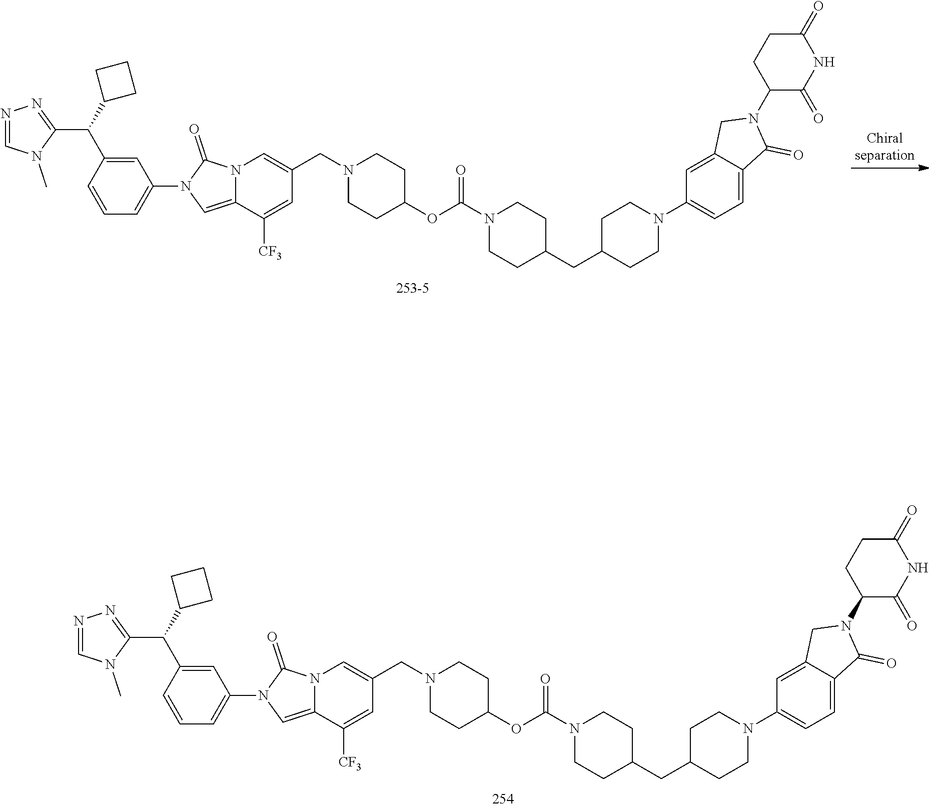 [SMILES: Cn1cnnc1[C@@H](c1cccc(-n2cc3c(C(F)(F)F)cc(CN4CCC(OC(=O)N5CCC(CC6CCN(c7ccc8c(c7)CN(C7CCC(=O)NC7=O)C8=O)CC6)CC5)CC4)cn3c2=O)c1)C1CCC1 ; Cn1cnnc1[C@@H](c1cccc(-n2cc3c(C(F)(F)F)cc(CN4CCC(OC(=O)N5CCC(CC6CCN(c7ccc8c(c7)CN([C@H]7CCC(=O)NC7=O)C8=O)CC6)CC5)CC4)cn3c2=O)c1)C1CCC1]

### Synthesis of Compound 254.

The Compound 253-5 (100 mg) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 80% B to 80% B in 9 min; Wave Length: 220/254 nm; RT1 (min): 4.92; RT2 (min): 7.08, the second peak is product) to afford the crude product. The crude product was purified by Prep-HPLC with the following conditions (Column: Xselect CSH C18 OBD Column 30*150 mm 5 μm, n; Mobile Phase A: water (0.1% FA), Mobile Phase B: ACN; Flow rate: 60 mL/min; Gradient: 25% B to 36% B in 7 min, Wave Length: 254; 220 nm; RT1 (min): 6.27) to afford Compound 253 (16.7 mg, 16.7%) as a yellow solid.

LC-MS-Compound 254: (ES, m/z): [M+H]⁺ 991

H-NMR-Compound 254: (400 MHz, DMSO-d₆, δ ppm): 0.93.99 (m, 2H), 1.11-1.16 (m, 4H), 1.56-1.82 (m, 15H), 1.90-1.99 (m, 1H), 2.03-2.13 (m, 1H), 2.31-2.34 (m, 2H), 2.55-2.60 (m, 4H), 2.76-2.81 (m, 5H), 3.24-3.27 (m, 3H), 3.43 (s, 3H), 3.83-3.86 (m, 2H), 3.95-3.97 (m, 2H), 4.16-4.33 (m, 3H), 4.59-4.65 (m, 1H), 5.01-5.04 (d, 1H), 7.01-7.03 (m, 3H), 7.17-7.19 (d, 1H), 7.32 (s, 1H), 7.41-7.50 (m, 2H), 7.67-7.73 (m, 3H), 8.32 (s, 1H), 10.95 (s, 1H).

### Example 253. Compound 255

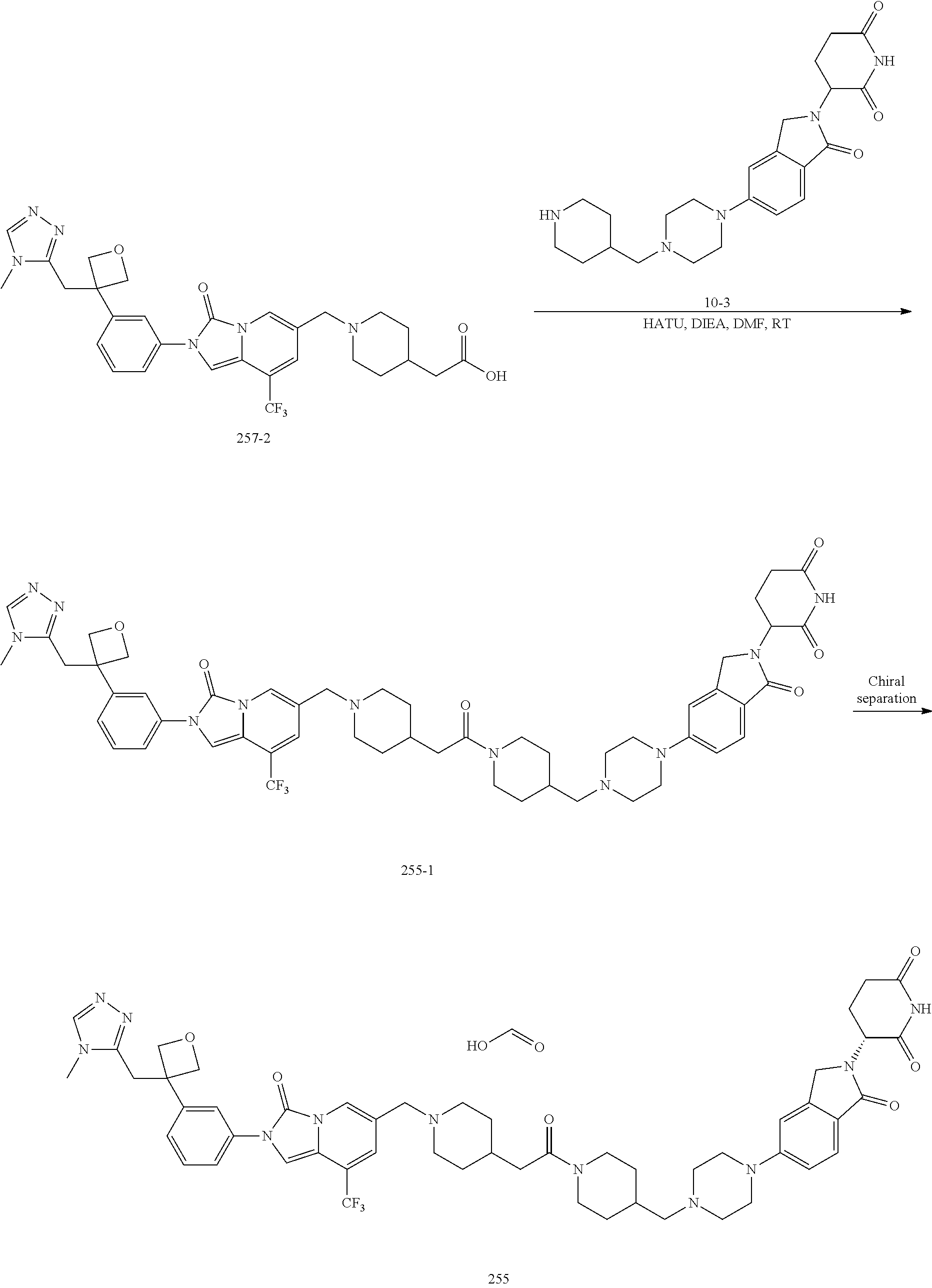

### Synthesis of Compound 255-1.

Into a 40 mL sealed tube were added Compound 257-2 (500 mg, 0.855 mmol, 1 equiv), Compound 10-3 (363.95 mg, 0.855 mmol, 1 equiv), DMF (5 mL), DIEA (221.09 mg, 1.710 mmol, 2 equiv) and HATU (487.81 mg, 1.282 mmol, 1.5 equiv) at room temperature. The mixture was stirred for overnight at room temperature. The reaction was quenched with 10% AcOH(aq.) (15 mL) at room temperature. The aqueous layer was extracted with CH₂Cl₂ (3×20 mL). The resulting mixture was concentrated under reduced pressure. The crude product (300 mg) was purified by Prep-HPLC with the following conditions (Column: CHIRALPAK IH, 2*25 cm, 5 μm, Mobile Phase A: MTBE (0.1% TEA)—HPLC, Mobile Phase B: DCM:ACN=1:1—HPLC; Flow rate: 20 mL/min; Gradient: 90% B to 90% B in 23 min; Wave Length: 220/254 nm; RT1 (min): 13.89; RT2 (min): 18.53; Sample Solvent: DCM:ACN=1:1—HPLC; Injection Volume: 2.2 mL; Number of Runs: 1) to afford Compound 255-1 (20 mg) as a yellow solid.

### Synthesis of Compound 255.

The Compound 255-1 (20 mg) was purified by Prep-Chiral-HPLC with the following conditions (Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 90% B to 90% B in 23 min; Wave Length: 220/254 nm; RT1 (min): 13.89; RT2 (min): 18.53; The first peak was product. Sample Solvent: DCM:ACN=1:1—HPLC; Injection Volume: 2.2 mL; Number of Runs: 1) to afford crude product. The crude product was purified by Prep-HPLC with the following conditions (Column: Sunfire prep C18 column, 30*150 mm, 5 μm, Mobile Phase A: Water (0.1% FA), Mobile Phase B: ACN; Flow rate: 60 mL/min; Gradient: 7% B to 20% B in 7 min, 20% B: Wave Length: 254; 220 nm; RT1 (min): 5.98) to afford Compound 255 (2.4 mg, 11.10%) as a yellow solid.

LC-MS-Compound 255: (ES, m/z): [M−HCOOH+H]⁺ 992

H-NMR-Compound 255: (400 MHz, CD₃OD, δ ppm): 0.94-1.21 (m, 2H), 1.33-1.50 (m, 2H), 1.82-1.99 (m, 6H), 2.06-2.25 (m, 1H), 2.35-2.40 (s, 4H), 2.42-2.48 (m, 2H), 2.57-3.01 (m, 11H), 3.08-3.22 (m, 3H), 3.35-3.48 (s, 4H), 3.51-3.80 (s, 4H), 3.88-4.15 (d, 1H), 4.25-4.49 (s, 2H), 4.49-4.70 (d, 1H), 5.04-5.13 (m, 5H), 6.86-6.96 (m, 1H), 7.08-7.11 (m, 1H), 7.12-7.18 (s, 2H), 7.27-7.30 (d, 1H), 7.36-7.55 (m, 1H), 7.58-7.69 (m, 2H), 7.75-7.81 (s, 1H), 8.05-8.28 (s, 1H).

### Example 254. Compound 256

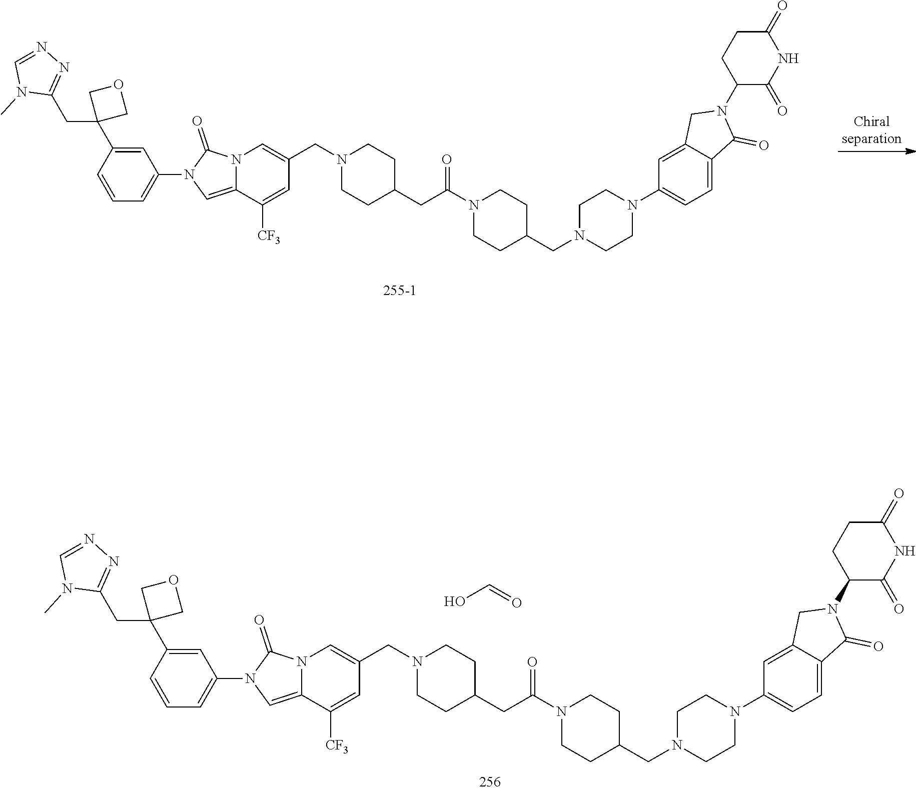

### Synthesis of Compound 256.

The Compound 255-1 (20 mg) was purified by Prep-Chiral-HPLC with the following conditions (Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 90% B to 90% B in 23 min; Wave Length: 220/254 nm; RT1 (min): 13.89; RT2 (min): 18.53; The second peak was the product. Sample Solvent: DCM:ACN=1:1—HPLC; Injection Volume: 2.2 mL; Number of Runs: 1) to afford crude product. The crude product was purified by Prep-HPLC with the following conditions (Column. Sunfire prep C18 column, 30*150 mm, 5 μm, Mobile Phase A: Water (0.1% FA), Mobile Phase B: ACN; Flow rate: 60 mL/min; Gradient: 7% B to 20% B in 7 min, 20% B; Wave Length: 254; 220 nm; RT1 (min): 6.27) to afford Compound 256 (2.2 mg, 10.80%) as a yellow solid.

LC-MS-Compound 256: (ES, m/z): [M−HCOOH+H]⁺ 992

H-NMR-Compound 256: (400 MHz, CD₃OD-d4, δ ppm): 0.98-1.28 (m, 2H), 1.36-1.57 (m, 2H), 1.70-2.06 (m, 6H), 2.06-2.25 (m, 1H), 2.32-2.42 (m, 4H), 2.47-2.55 (m, 2H), 2.61-2.81 (m, 6H), 2.81-3.02 (m, 4H), 3.10-3.26 (m, 3H), 3.35-3.45 (s, 4H), 3.55-3.85 (d, 4H), 3.88-4.15 (d, 1H), 4.25-4.48 (s, 2H), 4.48-4.72 (d, 2H), 5.00-5.20 (s, 5H), 6.80-7.00 (d, 2H), 7.06-7.10 (m, 1H), 7.11-7.18 (s, 2H), 7.21-7.38 (s, 1H), 7.38-7.55 (m, 1H), 7.55-7.72 (m, 2H), 7.72-7.94 (s, 1H), 8.05-8.23 (s, 1H).

### Example 255. Compound 257

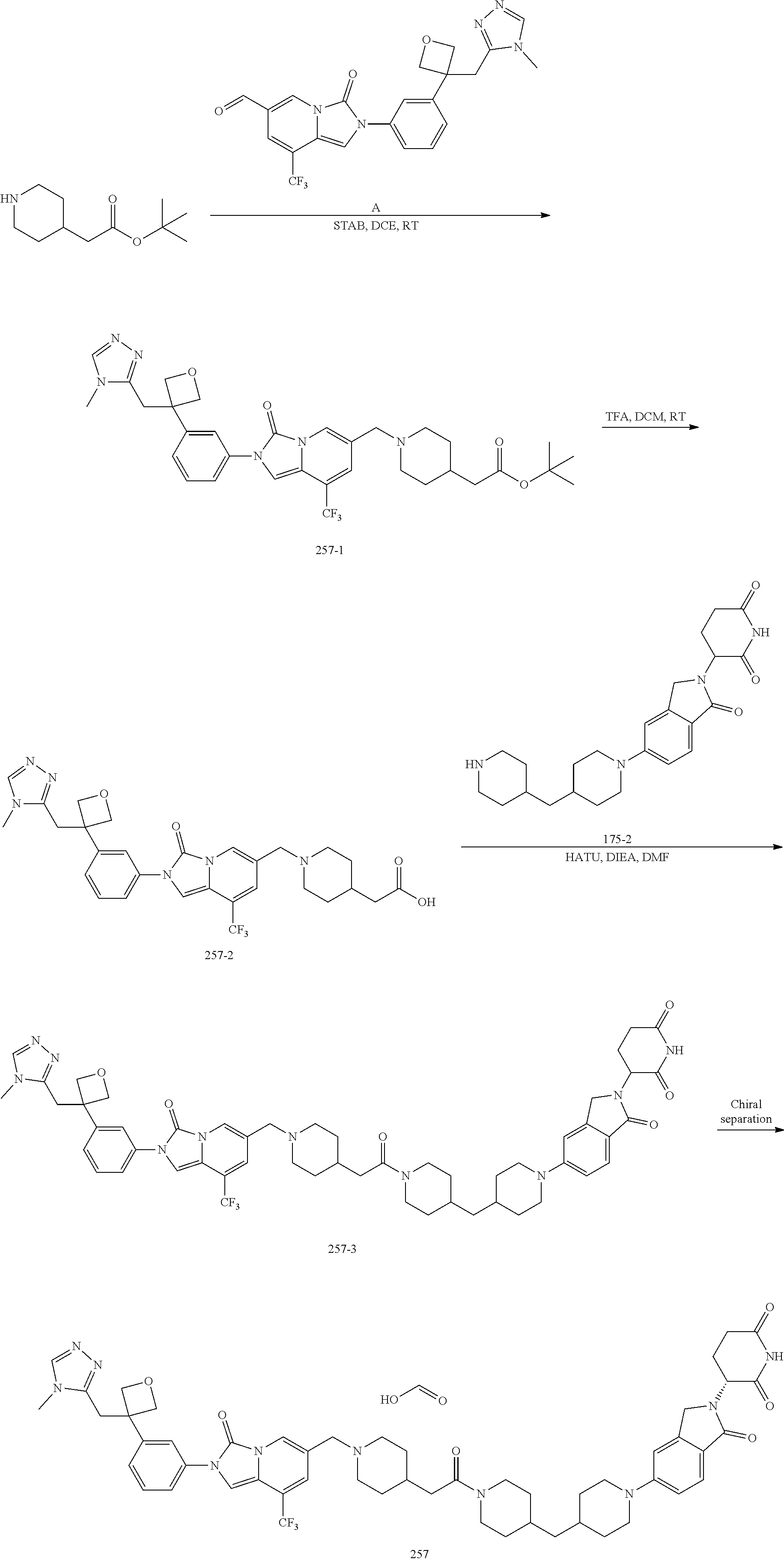

### Synthesis of Compound 257-1.

Into a 100 mL round-bottom flask were added tert-butyl 2-(piperidin-4-yl) acetate (2 g, 10.035 mmol, 1 equiv), Intermediate A (4.59 g, 10.035 mmol, 1 equiv), STAB (4.25 g, 20.070 mmol, 2 equiv) and DCE (20 mL) at room temperature. The mixture was stirred for overnight at room temperature. The reaction was quenched with water (50 ml) at room temperature. The aqueous layer was extracted with EtOAc (3×50 mL). The resulting mixture was concentrated under reduced pressure. The residue was purified by silica gel column chromatography, eluted with CH₂Cl₂/MeOH (50:1) to afford Compound 257-1 (1.5 g, 21.70%) as a yellow solid.

### Synthesis of Compound 257-2.

Into a 40 mL sealed tube were added Compound 257-1 (1.5 g, 2.341 mmol, 1 equiv), DCM (15 mL) and TFA (5 mL) at room temperature. The mixture was stirred for overnight at room temperature. The reaction was quenched with sat. NaHCO₃ (aq.) at room temperature. The aqueous layer was extracted with EtOAc (3×50 mL). The resulting mixture was concentrated under reduced pressure. The resulting product was used in the next step directly without further purification. This resulted in Compound 257-2 (1 g, 67.22%) as a yellow solid.

### Synthesis of Compound 257-3.

Into an 8 mL sealed tube were added Compound 257-2 (500 mg, 0.855 mmol, 1 equiv), Compound 175-2 (363.11 mg, 0.855 mmol, 1 equiv), DMF (5 mL), DIEA (221.09 mg, 1.710 mmol, 2 equiv) and HATU (487.81 mg, 1.282 mmol, 1.5 equiv) at room temperature. The mixture was stirred for overnight at room temperature. The reaction was quenched with 10% AcOH(aq.) (15 mL) at room temperature. The aqueous layer was extracted with EtOAc (3×20 mL). The resulting mixture was concentrated under reduced pressure. The crude product (300 mg) was purified by Prep-HPLC with the following conditions (Column: CHIRALPAK IH, 2*25 cm, 5 μm, Mobile Phase A: MTBE (0.1% FA)—HPLC, Mobile Phase B: DCM:ACN=1:1—HPLC; Flow rate: 20 mL/min; Gradient: 90% B to 90% B in 25 min; Wave Length: 220/254 nm; RT1 (min): 11.94; Sample Solvent: DCM:ACN=1:1—HPLC; Injection Volume: 2 mL; Number of Runs: 1) to afford Compound 257-3 (20 mg, 2.12%) as a yellow solid.

### Synthesis of Compound 257-0

The Compound 257-3 (20 mg) was purified by Prep-Chiral-HPLC with the following conditions (Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 90% B to 90% B in 25 min; Wave Length: 220/254 nm; RT1 (min): 11.94; RT2 (min): 18.30; The first peak was the product. Sample Solvent: DCM:ACN=1:1—HPLC; Injection Volume: 2 mL; Number of Runs: 1) to afford crude product. The crude product was purified by Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 18% B to 32% B in 7 min, 32% B; Wave Length: 254; 220 nm; RT (min): 6.3) to afford Compound 257 (2.8 mg, 13.33%) as a yellow solid.

LC-MS-Compound 257: (ES, m/z): [M−HCOOH+H]⁺ 991

H-NMR-Compound 257: (400 MHz, CD₃OD, δ ppm): 1.01-1.19 (m, 2H), 1.20-1.38 (m, 6H), 1.39-1.48 (m, 2H), 1.58-1.71 (s, 1H), 1.72-1.92 (m, 7H), 2.12-2.19 (m, 1H), 2.33-2.41 (s, 2H), 2.41-2.50 (s, 2H), 2.54-2.72 (m, 1H), 2.73-2.91 (m, 4H), 2.91-3.02 (s, 3H), 3.07-3.21 (m, 3H), 3.60-3.72 (s, 4H), 3.83-4.01 (m, 3H), 4.32-4.46 (s, 2H), 4.50-4.58 (m, 1H), 5.02-5.13 (s, 5H), 6.87-6.92 (s, 1H), 6.97-7.20 (m, 4H), 7.22-7.30 (s, 1H), 7.40-7.53 (m, 1H), 7.57-7.67 (m, 2H), 7.67-7.82 (s, 1H), 8.02-8.29 (s, 1H).

### Example 256. Compound 258

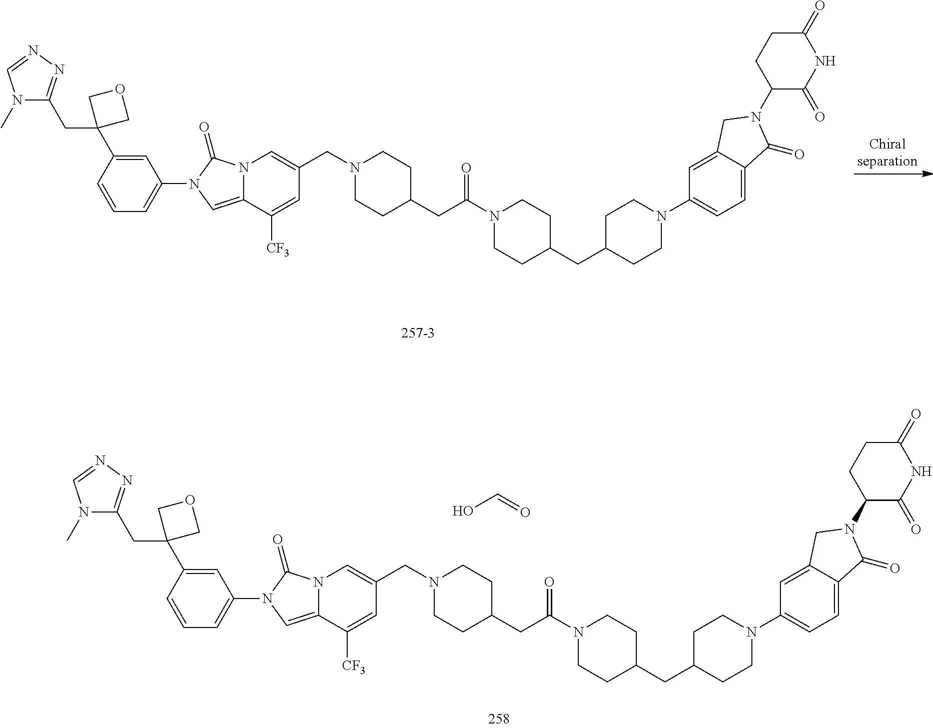 [SMILES: Cn1cnnc1CC1(c2cccc(-n3cc4c(C(F)(F)F)cc(CN5CCC(CC(=O)N6CCC(CC7CCN(c8ccc9c(c8)CN(C8CCC(=O)NC8=O)C9=O)CC7)CC6)CC5)cn4c3=O)c2)COC1 ; Cn1cnnc1CC1(c2cccc(-n3cc4c(C(F)(F)F)cc(CN5CCC(CC(=O)N6CCC(CC7CCN(c8ccc9c(c8)CN([C@H]8CCC(=O)NC8=O)C9=O)CC7)CC6)CC5)cn4c3=O)c2)COC1 ; O=CO]

### Synthesis of Compound 258.

The Compound 257-3 (20 mg) was purified by Prep-Chiral-HPLC with the following conditions (Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 90% B to 90% B in 25 min; Wave Length: 220/254 nm; RT1 (min): 11.94; RT2 (min): 18.30; The second peak was the product. Sample Solvent: DCM:ACN=1:1—HPLC; Injection Volume: 2 mL; Number of Runs: 1) to afford crude product. The crude product was purified by Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 18% B to 32% B in 7 min, 32% B: Wave Length: 254; 220 nm; RT1 (min): 6.3) to afford Compound 258-0 (2.6 mg, 12.96%) as a yellow solid.

LC-MS-Compound 258: (ES, m/z): [M−HCOOH+H]⁺ 991

H-NMR-Compound 258: (400 MHz, CD₃OD, δ ppm): 0.95-1.47 (m, 9H), 1.59-1.89 (m, 8H), 2.02-2.21 (m, 1H), 2.21-2.55 (m, 5H), 2.55-2.71 (m, 1H), 2.72-2.92 (m, 4H), 2.92-2.97 (s, 3H), 3.05-3.16 (m, 3H), 3.50-3.62 (s, 2H), 3.62-3.77 (s, 2H), 3.85-4.05 (m, 3H), 4.22-4.48 (d, 2H), 4.48-4.67 (d, 1H), 5.01-5.15 (s, 5H), 6.78-6.98 (d, 1H), 6.98-7.20 (m, 4H), 7.20-7.38 (s, 1H), 7.38-7.53 (m, 1H), 7.53-7.70 (m, 2H), 7.70-7.88 (s, 1H), 8.05-8.33 (s, 1H).

### Example 257. Compound 259

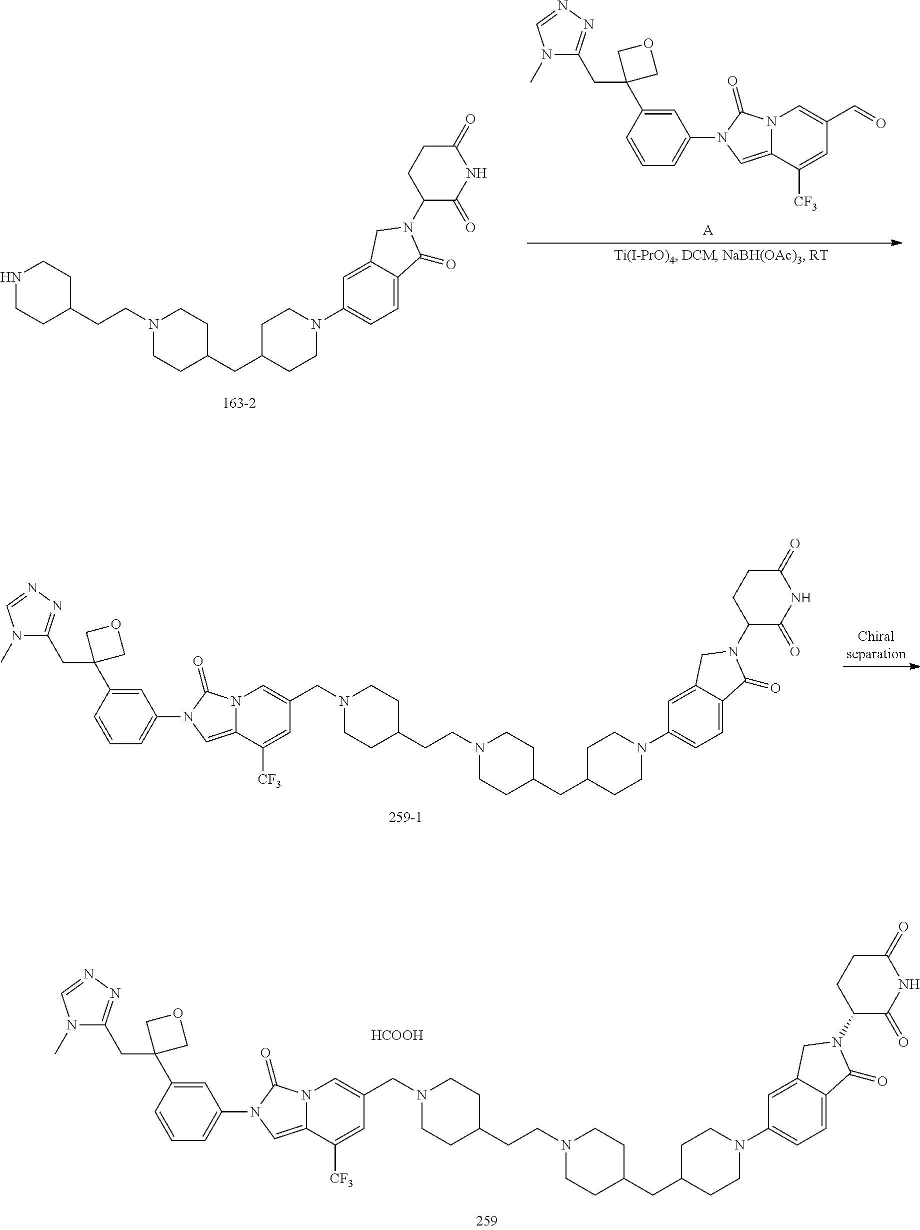

### Synthesis of Compound 259-1.

To a stirred solution of Compound 163-2 (1 g, 1.867 mmol, 1 equiv) and Intermediate A (1.20 g, 2.614 mmol, 1.4 equiv) in DCM (20 mL) was added Ti(i-PrO)₄ (1.06 g, 3.734 mmol, 2 equiv) at room temperature. The resulting mixture was stirred overnight at room temperature. To the above mixture was added NaBH(OAc)₃ (0.79 g, 3.734 mmol, 2 equiv) at room temperature. The resulting mixture was stirred for additional 4 h at room temperature. The reaction was quenched with of 10% HOAc(aq.) (200 mL) at room temperature. The resulting mixture was filtered, the filter cake was washed with 10% HOAc(aq.) (20 mL). The aqueous layer was extracted with EtOAc (100 mL). The aqueous layer was concentrated under vacuum. The crude product was purified by Prep-HPLC with the following conditions (Column: Sunfire prep C18 column, 30*150 mm, 5 μm; Mobile Phase A: Water (0.1% FA), Mobile Phase B: ACN; Flow rate: 60 mL/min; Gradient: 8% B to 19% B in 10 min, Wave Length: 254; 220 nm; RT1 (min): 9.53) to afford Compound 259-1 (200 mg, 10.97%) as a yellow solid.

### Synthesis of Compound 259-4.

The Compound 259-1 (200 mg) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 90% B to 90% B in 12 min; Wave Length: 220/254 nm; RT1 (min): 6.61; RT2 (min): 9.92, the first peak is product) to afford the crude product. The crude product was purified by Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 8% B to 19% B in 9 min, Wave Length: 254; 220 nm; RT1 (min): 8.78) to afford Compound 259 (23.8 mg, 11.9%) as a yellow solid.

LC-MS-Compound 259: (ES, m/z): [M−HCOOH+H]⁺ 977

H-NMR-Compound 259: (400 MHz, DMSO-d6, δ ppm): 1.12-1.28 (m, 9H), 1.38-1.40 (m, 3H), 1.63-1.71 (m, 7H), 1.95-2.09 (m, 5H), 2.37-2.49 (m, 3H), 2.55-2.65 (m, 1H), 2.72-2.96 (m, 10H), 3.25 (s, 2H), 3.53 (s, 2H), 3.84-3.87 (m, 2H), 4.20-4.25 (d, 1H), 4.33-4.37 (d, 1H), 4.90-4.95 (m, 4H), 5.01-5.05 (d, 1H), 6.88-6.90 (d, 1H), 7.00-7.03 (m, 3H), 7.30 (s, 1H), 7.37-7.41 (m, 2H), 7.47-7.50 (d, 1H), 7.73 (s, 1H), 7.76-7.78 (d, 1H), 8.19 (s, 2H), 10.95 (s, 1H).

### Example 258. Compound 260

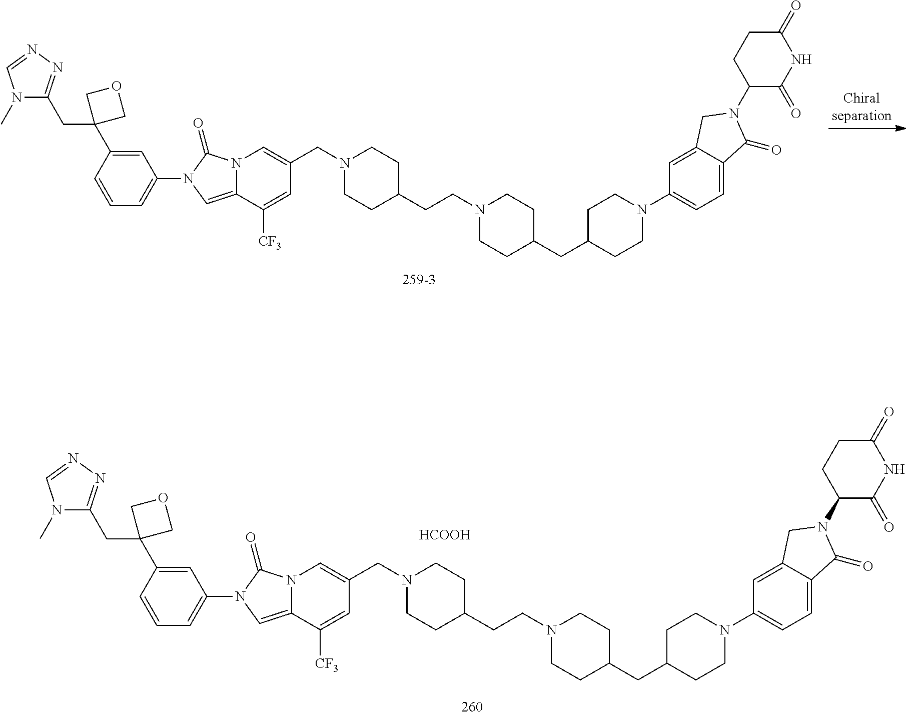 [SMILES: Cn1cnnc1CC1(c2cccc(-n3cc4c(C(F)(F)F)cc(CN5CCC(CCN6CCC(CC7CCN(c8ccc9c(c8)CN(C8CCC(=O)NC8=O)C9=O)CC7)CC6)CC5)cn4c3=O)c2)COC1 ; Cn1cnnc1CC1(c2cccc(-n3cc4c(C(F)(F)F)cc(CN5CCC(CCN6CCC(CC7CCN(c8ccc9c(c8)CN([C@H]8CCC(=O)NC8=O)C9=O)CC7)CC6)CC5)cn4c3=O)c2)COC1 ; "HCOOH"]

### Synthesis of Compound 260.

The Compound 259-3 (200 mg) was purified by Prep-Chiral-HPLC with the following conditions (Column: CHIRALPAK IH, 2*25 cm, 5 μm; Mobile Phase A: MTBE (0.1% TEA), Mobile Phase B: DCM:ACN=1:1; Flow rate: 20 mL/min; Gradient: 90% B to 90% B in 12 min; Wave Length: 220/254 nm; RT1 (min): 6.61; RT2 (min): 9.92, the second peak is product) to afford the crude product. The crude product was purified by Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 7% B to 18% B in 11 min, Wave Length: 254; 220 nm; RT1 (min): 10.73) to afford Compound 260 (8.3 mg, 4.15%) as a yellow solid.

LC-MS-Compound 260: (ES, m/z): [M−HCOOH+H]⁺ 977

H-NMR-Compound 260: (400 MHz, DMSO-d₆, δ ppm): 1.12-1.28 (m, 9H), 1.38-1.40 (m, 3H), 1.63-1.71 (m, 7H), 1.95-1.98 (m, 3H), 2.01-2.19 (m, 2H), 2.37-2.49 (m, 3H), 2.55-2.65 (m, 1H), 2.72-2.96 (m, 10H), 3.25 (s, 2H), 3.53 (s, 2H), 3.84-3.87 (m, 2H), 4.20-4.25 (d, 1H), 4.33-4.37 (d, 1H), 4.90-4.95 (m, 4H), 5.01-5.05 (d, 1H), 6.88-6.90 (d, 1H), 7.00-7.03 (m, 3H), 7.30-7.41 (m, 3H), 7.47-7.50 (d, 1H), 7.73 (s, 1H), 7.76-7.78 (d, 1H), 8.19 (s, 1H), 8.21 (s, 1H), 10.95 (s, 1H).

### Example 259. Compound 261

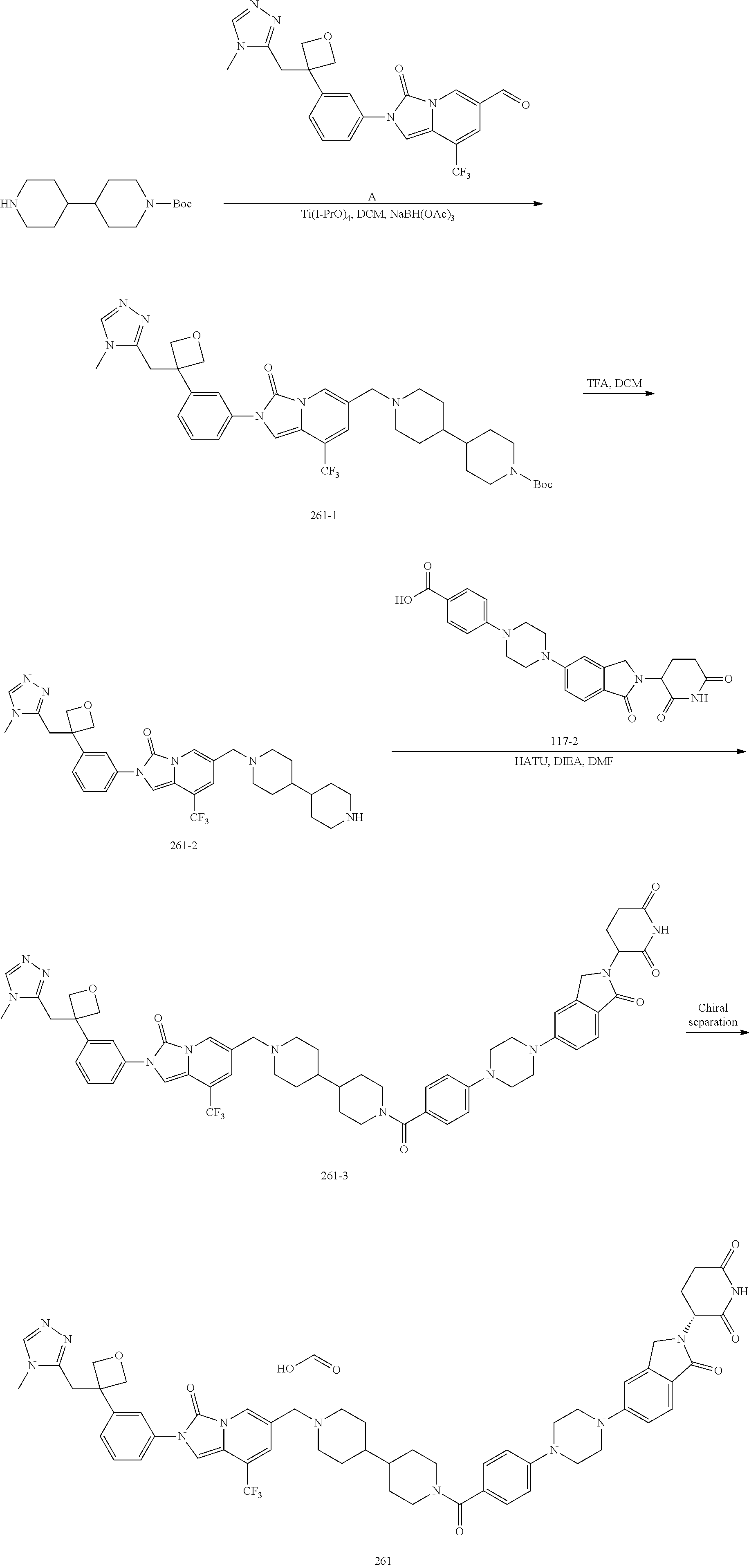

### Synthesis of Compound 261-1.

To a stirred mixture of tert-butyl [4,4′-bipiperidine]-1-carboxylate (586 mg, 2.183 mmol, 1 equiv) and Intermediate A (1.00 g, 2.183 mmol, 1.00 equiv) in DCM (10 mL) were added NaBH(OAc)₃ (1.39 g, 6.549 mmol, 3 equiv) and tetrakis(propan-2-yloxy) titanium (2.48 g, 8.732 mmol, 4 equiv) at room temperature under nitrogen atmosphere. The resulting mixture was stirred for overnight at room temperature. The reaction was quenched by the addition of water (20 mL) at room temperature. The aqueous layer was extracted with CH₂Cl₂ (3×10 mL). The resulting mixture was concentrated under reduced pressure. The residue was purified by silica gel column chromatography, eluted with CH₂Cl₂/MeOH (100:1) to afford Compound 261-1 (400 mg, 24.0)%) as a yellow solid.

### Synthesis of Compound 261-2.

A solution of Compound 261-1 (400 mg, 0.564 mmol, 1 equiv) in TFA (1 mL) and DCM (3 mL) was stirred for 2 h at room temperature. The resulting mixture was concentrated under reduced pressure. This resulted in Compound 261-2 (260 mg, 71.89%) as a yellow solid. The resulting mixture was used in the next step directly without further purification.

### Synthesis of Compound 261-3.

To a stirred mixture of Compound 261-2 (260 mg, 0.426 mmol, 1 equiv) and Compound 117-2 (229.50 mg, 0.511 mmol, 1.2 equiv) in DMF (3 mL) were added HATU (405.37 mg, 1.065 mmol, 2.5 equiv) and DIEA (137.79 mg, 1.065 mmol, 2.5 equiv) at room temperature. The resulting mixture was stirred for overnight at room temperature. The reaction was quenched by the addition of water (10 mL) at room temperature. The aqueous layer was extracted with CH₂Cl₂ (3×10 mL). The resulting mixture was concentrated under reduced pressure. The residue was purified by Prep-TLC (CH₂Cl₂/MeOH 20:1) to afford crude product (100 mg). The crude product (100 mg) was purified by Prep-HPLC with the following conditions (Column: Sunfire prep C18 column, 30*150 mm, 5 μm; Mobile Phase A: Water (0.1% FA), Mobile Phase B: ACN; Flow rate: 60 mL/min; Gradient: 20% B to 30% B in 9 min, 30% B: Wave Length: 254; 220 nm; RT1 (min): 8.3) to afford Compound 261-3 (60 mg, 12.85%) as a yellow solid.

### Synthesis of Compound 261.

The Compound 261-3 (60 mg) was purified by Prep-Chiral-HPLC with the following conditions (Column: CHIRALPAK IH, 2*25 cm, 5 μm; Mobile Phase A: Hex (0.2% TEA)—HPLC, Mobile Phase B: IPA:DCM=1:1—HPLC; Flow rate: 20 mL/min; Gradient: 90% B to 90% B in 15 min; Wave Length: 220/254 nm; RT1 (min): 5.62; RT2 (min): 11.69; The first peak was the product; Sample Solvent: IPA:DCM=1:1—HPLC; Injection Volume: 1.6 mL; Number of Runs: 2) to afford crude product. The crude product was purified by reversed-phase flash chromatography with the following conditions: column, 40 g C18 silica gel; mobile phase, MeCN in water (0.1% FA), 20% to 45% gradient in 8 min; detector, UV 254 nm to afford Compound 261 (11.3 mg, 18.19%) as a yellow solid.

LC-MS-Compound 261: (ES, m/z): [M−HCOOH+H]⁺ 1040

H-NMR-Compound 261: (400 MHz, CD₃OD, ppm): δ 1.20-1.25 (m, 4H), δ1.30-1.48 (m, 3H), δ1.71-1.91 (m, 4H), δ2.01-2.15 (m, 1H), δ2.32-2.45 (m, 3H), δ2.75-2.76 (m, 1H), δ2.82-2.86 (m, 1H), δ2.90 (s, 3H), δ3.17-3.20 (d, 1H), δ3.26-3.27 (d, 2H), δ3.40-3.41 (d, 4H), δ3.46-3.49 (m, 4H), δ3.62 (s, 4H), δ3.81-3.98 (m, 1H), δ4.37-4.39 (d, 2H), δ4.50-4.65 (m, 1H), δ5.01-5.09 (m, 5H), δ6.86-6.88 (d, 1H), δ7.00-7.02 (d, 2H), δ7.07-7.11 (m, 4H), δ7.24 (s, 1H), δ7.30-7.32 (d, 2H), δ7.40-7.44 (m, 1H), δ7.57-7.59 (d, 1H), δ7.59-7.61 (d, 1H), δ7.63 (s, 1H), δ7.75 (s, 1H), δ8.35 (s, 1H).

### Example 260. Compound 262

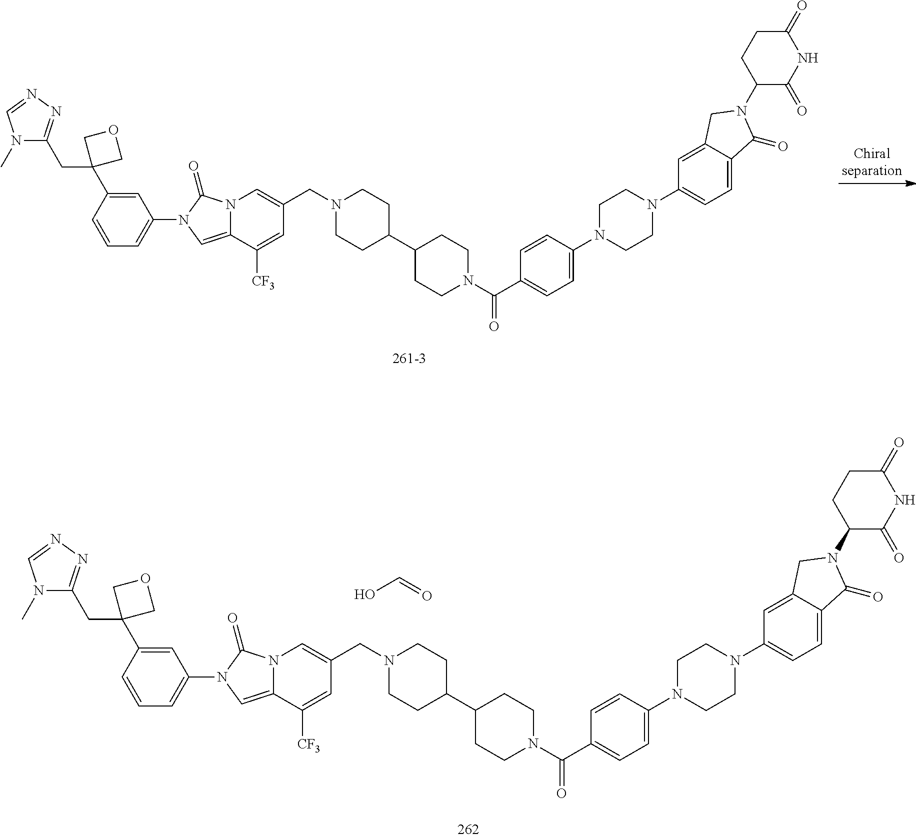 [SMILES: Cn1cnnc1CC1(c2cccc(-n3cc4c(C(F)(F)F)cc(CN5CCC(C6CCN(C(=O)c7ccc(N8CCN(c9ccc%10c(c9)CN(C9CCC(=O)NC9=O)C%10=O)CC8)cc7)CC6)CC5)cn4c3=O)c2)COC1 ; Cn1cnnc1CC1(c2cccc(-n3cc4c(C(F)(F)F)cc(CN5CCC(C6CCN(C(=O)c7ccc(N8CCN(c9ccc%10c(c9)CN([C@H]9CCC(=O)NC9=O)C%10=O)CC8)cc7)CC6)CC5)cn4c3=O)c2)COC1 ; O=CO]

### Synthesis of Compound 262.

The Compound 261-3 (60 mg) was purified by Prep-Chiral-HPLC with the following conditions (Column: CHIRALPAK IH, 2*25 cm, 5 μm; Mobile Phase A: Hex (0.2% TEA)—HPLC, Mobile Phase B: IPA:DCM=1:1—HPLC; Flow rate: 20 mL/min; Gradient: 90% B to 90% B in 15 min; Wave Length: 220/254 nm; RT1 (min): 5.62; RT2 (min): 11.69; The second peak was the product; Sample Solvent: IPA:DCM=1:1—HPLC; Injection Volume: 1.6 mL; Number of Runs: 2) to afford crude product. The crude product was purified by reversed-phase flash chromatography with the following conditions: column, 40 g C18 silica gel; mobile phase, MeCN in water (0.1% FA), 20% to 45% gradient in 8 min; detector, UV 254 nm to afford Compound 262 (9 mg, 15.00%) as a yellow solid.

LC-MS-Compound 262: (ES, m/z): [M−HCOOH+H]⁺ 1040

H-NMR-Compound 262: (400 MHz, CD₃OD, ppm): δ1.18-1.29 (m, 4H), δ1.32-1.48 (m, 3H), δ1.71-1.91 (m, 4H), δ2.01-2.15 (m, 1H), δ2.32-2.45 (m, 3H), δ2.75-2.76 (m, 1H), δ2.82-2.86 (m, 1H), δ2.90 (s, 3H), δ3.17-3.20 (d, 1H), δ3.26-3.27 (d, 2H), δ3.40-3.41 (d, 4H), δ3.46-3.49 (m, 4H), δ3.62 (s, 4H), δ3.81-3.98 (m, 1H), δ4.37-4.39 (d, 2H), δ4.50-4.65 (m, 1H), δ5.01-5.09 (m, 5H), δ6.86-6.88 (d, 1H), δ7.00-7.02 (d, 2H), δ7.07-7.11 (m, 4H), δ7.24 (s, 1H), δ7.30-7.32 (d, 2H), δ7.40-7.44 (m, 1H), δ7.57-7.59 (d, 1H), δ7.55-7.57 (d, 1H), δ7.67 (s, 1H), δ8.32 (s, 1H).

### Example 261. Compound 263

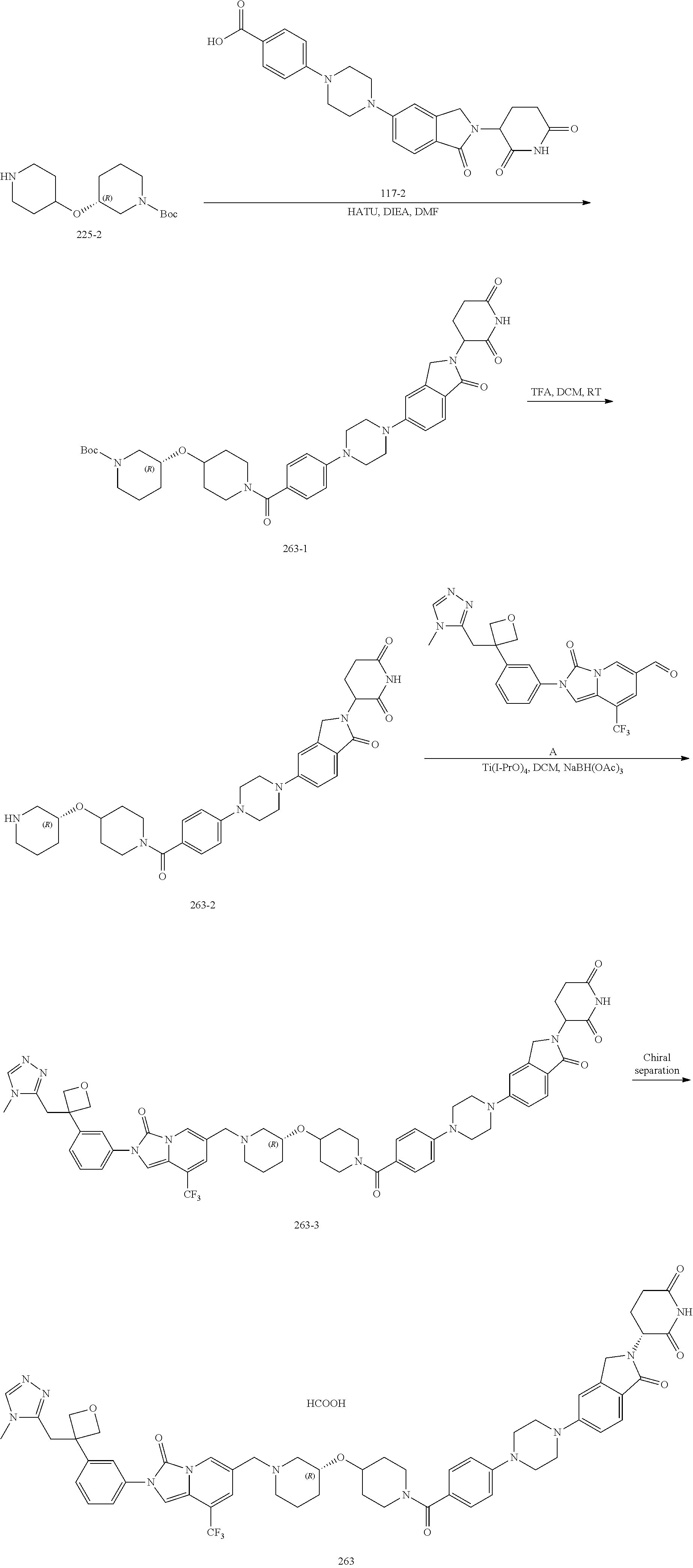

### Synthesis of Compound 263-1.

To a stirred mixture of Compound 225-2 (7 g, 24.61 mmol, 4.0 equiv) and Compound 117-2 (2.8 g, 6.15 mmol, 1.0 equiv) in DMF (100 mL) were added HATU (5.9 g, 15.38 mmol, 2.5 equiv) and DIEA (2 g, 15.38 mmol, 2.5 equiv) at room temperature under nitrogen atmosphere. The resulting mixture was stirred for overnight at room temperature under nitrogen atmosphere. The reaction was quenched by the addition of water (200 mL) at room temperature. The aqueous layer was extracted with CH₂CO₂ (3×100 mL). The resulting mixture was concentrated under reduced pressure. The residue was purified by silica gel column chromatography, eluted with CH₂Cl₂/MeOH (40:1) to afford Compound 263-1 (1.3 g, 28%) as a yellow solid.

### Synthesis of Compound 263-2.

A solution of Compound 263-1 (1.3 g, 1.40 mmol, 1.0 equiv) in TFA (3 mL) and DCM (9 mL) was stirred for 2 h at room temperature under nitrogen atmosphere. The resulting mixture was concentrated under reduced pressure. This resulted in Compound 263-2 (1 g, 85%) as a yellow solid.

### Synthesis of Compound 263-3.

To a stirred solution of Compound 263-2 (880 mg, 1.43 mmol, 1.0 equiv) and Intermediate A (785 mg, 1.71 mmol, 1.2 equiv) in DCM (9 mL) was added Ti(Oi-Pr)₄ (1.6 g, 5.72 mmol, 4.0 equiv) at room temperature. The resulting mixture was stirred for overnight at room temperature. To the above mixture was added NaBH(OAc)₃ (910 mg, 4.29 mmol, 3.0 equiv) at room temperature. The resulting mixture was stirred for additional 2 h at room temperature. The reaction was quenched by the addition of 10% AcOH (100 mL) at room temperature. The aqueous layer was extracted with CH₂Cl₂:MeOH (10:1) (3×100 mL). The resulting mixture was concentrated under reduced pressure. The residue was purified by reversed-phase flash chromatography with the following conditions: column, C18 silica gel; mobile phase, MeCN in Water (0.1% FA), 10% to 50% gradient in 30 min; detector, UV 254 nm. The crude product (700 mg) was purified by Prep-HPLC with the following conditions (Column: Sunfire prep C18 column, 30*150 mm, 5 μm; Mobile Phase A: Water (0.1% FA), Mobile Phase B: ACN; Flow rate: 60 mL/min; Gradient: 17% B to 30% B in 7 min, 30% B; Wave Length: 254; 220 nm; RT1 (min): 6.52; Number of Runs: 0) to afford Compound 263-3 (450 mg, 30%) as a yellow solid.

### Synthesis of Compound 263.

The Compound 263-3 (450 mg) was purified by Prep-CHIRAL-HPLC with the following conditions Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 90% B to 90% B in 35 min; Wave Length: 220/254 nm; RT1 (min): 12.04; RT2 (min): 23.48; Sample Solvent: DCM:ACN=1:1—HPLC; Injection Volume: 1.1 mL; Number Of Runs: 4. The first peak was the product. The residue was purified by reversed-phase flash chromatography with the following conditions: column, C18 silica gel; mobile phase, MeCN in water (0.1% FA), 10% to 50% gradient in 30 min; detector, UV 254 nm. This resulted in Compound 263 (182 mg, 38%) as a yellow solid.

LC-MS-Compound 263: (ES, m/z): [M-COOH]⁺ 1056

H-NMR-Compound 263: (400 MHz, DMSO-d6, δ ppm): 1.02-1.10 (m, 1H), 1.35-1.39 (m, 3H), 1.69-1.72 (m, 1H), 1.81-1.89 (m, 3H), 1.96-1.98 (m, 3H), 2.38-2.45 (m, 1H), 2.50-2.61 (m, 2H), 2.86-2.96 (m, 5H), 3.17-3.19 (m, 2H), 3.20-3.25 (m, 1H), 3.29-3.32 (m, 5H), 3.49-3.52 (m, 7H), 3.67-3.70 (m, 3H), 4.20-4.25 (d, 1H), 4.33-4.37 (d, 1H), 4.90-4.95 (m, 4H), 5.04-5.06 (m, 1H), 6.87-6.89 (d, 1H), 6.98-7.01 (m, 3H), 7.11-7.14 (m, 2H), 7.27-7.31 (m, 3H), 7.37-7.41 (m, 2H), 7.54-7.56 (d, 1H), 7.67 (s, 1H), 7.74-7.76 (d, 1H), 8.14 (s, 1H), 8.19 (s, 1H), 10.95 (s, 1H).

### Example 262. Compound 264

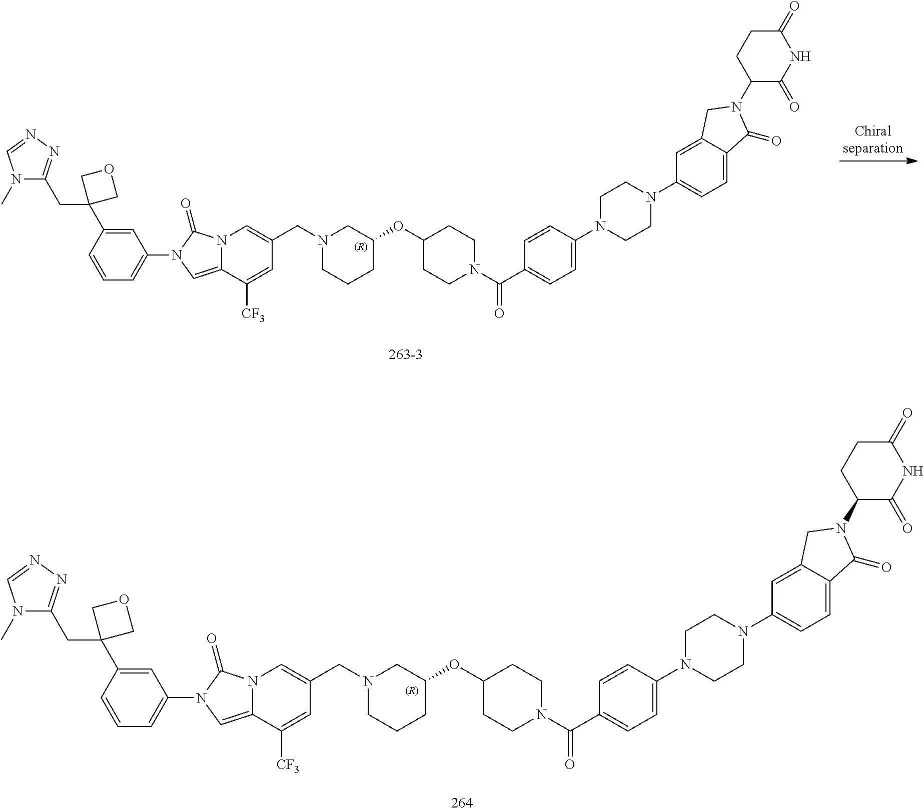

### Synthesis of Compound 264.

The Compound 263-3 (450 mg) was purified by Prep-CHIRAL-HPLC with the following conditions Column: CHIRALPAK IH, 2*25 cm, 5 μm; Mobile Phase A: MTBE (0.1% TEA)—HPLC, Mobile Phase B: DCM:ACN=1:1—HPLC; Flow rate: 20 mL/min; Gradient: 90% B to 90% B in 35 min; Wave Length: 220/254 nm; RT1 (min): 12.04; RT2 (min): 23.48; Sample Solvent: DCM:ACN=1:1—HPLC; Injection Volume: 1.1 mL; Number Of Runs: 4. The second peak was the product. The residue was purified by reversed-phase flash chromatography with the following conditions: column, C18 silica gel; mobile phase, MeCN in water (0.1% FA), 10% to 50% gradient in 30 min; detector, UV 254 nm. This resulted in Compound 264 (160 mg, 34%) as a yellow solid.

LC-MS-Compound 264: (ES, m/z): [M-COOH]⁺ 1056

H-NMR-Compound 264: (400 MHz, DMSO-d6, δ ppm): 1.02-1.10 (m, 1H), 1.35-1.39 (m, 3H), 1.69-1.72 (m, 1H), 1.81-1.89 (m, 3H), 1.96-1.98 (m, 3H), 2.38-2.45 (m, 1H), 2.50-2.61 (m, 2H), 2.86-2.96 (m, 5H), 3.17-3.19 (m, 2H), 3.20-3.25 (m, 1H), 3.29-3.32 (m, 5H), 3.49-3.52 (m, 7H), 3.67-3.70 (m, 3H), 4.20-4.25 (d, 1H), 4.33-4.37 (d, 1H), 4.90-4.95 (m, 4H), 5.04-5.06 (m, 1H), 6.87-6.89 (d, 1H), 6.98-7.01 (m, 3H), 7.11-7.14 (m, 2H), 7.27-7.31 (m, 3H), 7.37-7.41 (m, 2H), 7.54-7.56 (d, 1H), 7.67 (s, 1H), 7.74-7.76 (d, 1H), 8.14 (s, 1H), 8.19 (s, 1H), 10.95 (s, 1H).

LC-MS-Compound 264: (ES, m/z): [M-COOH]⁺ 1056

H-NMR-Compound 264: (400 MHz, DMSO-d6, δ ppm): 1.02-1.10 (m, 1H), 1.35-1.39 (m, 3H), 1.69-1.72 (m, 1H), 1.81-1.89 (m, 3H), 1.96-1.98 (m, 3H), 2.38-2.45 (m, 1H), 2.50-2.61 (m, 2H), 2.86-2.96 (m, 5H), 3.17-3.19 (m, 2H), 3.20-3.25 (m, 1H), 3.29-3.32 (m, 5H), 3.49-3.52 (m, 7H), 3.67-3.70 (m, 3H), 4.20-4.25 (d, 1H), 4.33-4.37 (d, 1H), 4.90-4.95 (m, 4H), 5.04-5.06 (m, 1H), 6.87-6.89 (d, 1H), 6.98-7.01 (m, 3H), 7.11-7.14 (m, 2H), 7.27-7.31 (m, 3H), 7.37-7.41 (m, 2H), 7.54-7.56 (d, 1H), 7.67 (s, 1H), 7.74-7.76 (d, 1H), 8.14 (s, 1H), 8.19 (s, 1H), 10.95 (s, 1H).

### Example 263. MSD-Based Assay for Degradation of Cbl-B in THP-1 Cells

On day one, PROTACs were serially diluted using a Tecan Freedom EVO200 and 100 nl of each dilution were acoustically transferred to a 96-well assay plate (Corning, 3894) using a Labcyte Echo 655. Jurkat cells were then seeded into the 96-well plates at 600,000 cells were well in 100 μL of RPMI 1640 medium and 0% FBS. Assay plates were then shaken at 600 rpm for 5 minutes, followed by incubation for 24 hours in the 37° C. TC incubator. After 24 hours, cells were spun down for 5 minutes, media discarded, followed by addition of 100 uL/well of RIPA lysis buffer containing protease inhibitor cocktail (Roche, 4693132001) and shaking at 600 rpm at 4° C. for about 20 minutes. Cell debris was then spun down at maximum speed (about 3200 g) for 30 min and lysates frozen in the −80° C. fridge. Meanwhile, MSD plates (L15XA-3) were coated overnight at 4° C. with 1 ug/mL capture antibody (Anti-Cbl-B antibody, 246C5a and Mouse mAB, ab54362). On day two, MSD coated bare plates were washed 3× with 150 ul/well of 1×TBST (CST #9997S), then blocked with 150 ul of blocking buffer/well while shaking for 1 hr at RT, 600 rpm. Blocking buffer was 3% Blocker A (MSD, R93BA-4) in TBST. Plates were washed 3× with 150 ul/well of 1×TBST and sample lysates (70 ul/well) then added, followed by shaking for 1 hr at RT, 600 rpm. MSD plates were then washed 3× with 150 ul/well of 1×TBST and then 25 ul/well of the Primary Detection antibody (Rabbit Anti-CBL-B antibody, cst-9498S) was added to a final concentration of 1 ug/ml and 25 uL/well, followed by shaking for 1 hr at RT, 600 rpm. Plates were again washed 3× with 150 ul/well of 1×TBST, followed by addition of Secondary Detection antibody, SULFO-TAG anti-species antibody (Anti Rabbit Antibody (R32AB-5) MSD, R32AB-1) at a final concentration of 1 ug/ml and 25 uL/well, followed by shaking for 1 hr at RT, 600 rpm. Plates were again washed 3× with 150 ul/well of 1×TBST, followed by addition of 1×MSD reading buffer (MSD, R92TC-2), 150 ul/well, diluted from 4× with water. Percent Cbl-B remaining and DC50s were calculated by fitting the Curves using Xlfit (v5.3.1.3), equation 201: Y=Bottom+(Top−Bottom)/(1+10\hat{ }((Log IC50−X)*HillSlope)).

Compound DC₅₀s were graded into A through E as follows: A indicates <30 nM, B indicates 30 nM-300 nM, C indicates 301 nM-1,000 nM, D indicates 1,001 nM-1000 nM, E indicates >10,000 nM. Compound Dmax were binned into A through D as follows: A indicates >70%, B indicates 50-70%, C indicates 30-50%, D indicates <30%.

TABLE 2

| | DC50 (nM) | Dmax |
|---|---|---|
| | (A < 30; 30 <= B < 300; | (A >= 70; |
| | 300 <= C < 1000; | 70 > B >= 50; |
| Cmpd | 1000 <= D < 10000; | 50 > C >= 30; |
| No | 10000 <= E) | 30 > D) |
| 1 | E | D |
| 2 | E | D |
| 3 | C | B |
| 4 | E | D |
| 5 | E | D |
| 6 | B | B |
| 7 | E | D |
| 8 | B | B |
| 9 | B | A |
| 10 | D | D |
| 11 | E | D |
| 12 | E | D |
| 13 | D | D |
| 14 | D | C |
| 15 | E | D |
| 16 | D | C |
| 17 | B | B |
| 18 | E | D |
| 19 | E | D |
| 20 | C | B |
| 21 | D | C |
| 22 | B | A |
| 23 | E | D |
| 24 | E | D |
| 25 | C | C |
| 26 | D | D |
| 27 | E | D |
| 28 | E | D |
| 29 | E | D |
| 30 | E | D |
| 31 | B | B |
| 32 | B | C |
| 33 | E | D |
| 34 | B | A |
| 35 | C | B |
| 36 | D | D |
| 37 | D | D |
| 38 | D | B |
| 39 | E | D |
| 40 | D | D |
| 41 | D | D |
| 42 | E | D |
| 43 | E | D |
| 44 | E | C |
| 45 | C | B |
| 46 | E | C |
| 47 | C | B |
| 48 | C | C |
| 49 | C | B |
| 50 | B | A |
| 51 | C | B |
| 52 | B | A |
| 53 | D | B |
| 54 | B | A |
| 55 | D | C |
| 56 | B | A |
| 57 | D | B |
| 58 | B | A |
| 59 | C | C |
| 60 | C | C |
| 61 | B | B |
| 62 | B | A |
| 63 | C | C |
| 64 | A | A |
| 65 | C | B |
| 66 | B | B |
| 67 | E | D |
| 68 | E | D |
| 69 | C | C |
| 70 | B | B |
| 71 | E | D |
| 72 | E | C |
| 73 | D | C |
| 74 | D | B |
| 75 | C | B |
| 76 | B | B |
| 77 | C | C |
| 78 | A | B |
| 79 | D | C |
| 80 | A | B |
| 81 | E | D |
| 82 | A | C |
| 83 | E | D |
| 84 | B | C |
| 85 | E | C |
| 86 | D | C |
| 87 | C | C |
| 88 | A | A |
| 89 | D | C |
| 90 | D | C |
| 91 | C | C |
| 92 | A | A |
| 93 | B | B |
| 94 | A | A |
| 95 | B | B |
| 96 | B | C |
| 97 | D | C |
| 98 | E | D |
| 99 | E | D |
| 100 | E | D |
| 101 | B | B |
| 102 | B | A |
| 103 | C | B |
| 104 | B | B |
| 105 | B | A |
| 106 | C | B |
| 107 | B | B |
| 108 | A | B |
| 109 | B | B |
| 110 | A | A |
| 111 | B | B |
| 112 | A | A |
| 113 | E | D |
| 114 | E | D |
| 115 | E | D |
| 116 | A | B |
| 117 | D | C |
| 118 | A | A |
| 119 | E | D |
| 120 | B | B |
| 121 | B | B |
| 122 | A | A |
| 123 | A | A |
| 124 | A | A |
| 125 | B | B |
| 126 | A | A |
| 127 | E | D |
| 128 | A | B |
| 129 | E | D |
| 130 | B | B |
| 131 | E | C |
| 132 | A | B |
| 133 | B | A |
| 134 | A | A |
| 135 | D | D |
| 136 | B | C |
| 137 | B | C |
| 138 | C | D |
| 139 | C | D |
| 140 | B | C |
| 141 | E | D |
| 142 | E | C |
| 143 | C | C |
| 144 | A | B |
| 145 | E | C |
| 146 | E | D |
| 147 | E | D |
| 148 | B | C |
| 149 | B | B |
| 150 | E | D |
| 151 | B | B |
| 152 | A | A |
| 153 | E | D |
| 154 | C | C |
| 155 | E | D |
| 156 | A | B |
| 157 | E | D |
| 158 | A | A |
| 159 | E | D |
| 160 | A | A |
| 161 | C | D |
| 162 | A | A |
| 163 | E | D |
| 164 | A | A |
| 165 | D | C |
| 166 | A | A |
| 167 | D | D |
| 168 | E | D |
| 169 | B | C |
| 170 | B | B |
| 171 | A | B |
| 172 | A | A |
| 173 | D | C |
| 174 | A | A |
| 175 | E | D |
| 176 | A | A |
| 177 | C | C |
| 178 | A | A |
| 179 | C | C |
| 180 | A | A |
| 181 | C | C |
| 182 | A | C |
| 183 | B | B |
| 184 | A | A |
| 185 | B | B |
| 186 | A | A |
| 187 | E | D |
| 188 | C | C |
| 189 | E | D |
| 190 | C | C |
| 191 | B | B |
| 192 | D | C |
| 193 | E | D |
| 194 | B | C |
| 195 | B | B |
| 196 | A | A |
| 197 | E | D |
| 198 | E | D |
| 199 | B | C |
| 200 | A | A |
| 201 | B | C |
| 202 | A | A |
| 203 | A | A |
| 204 | A | A |
| 205 | B | C |
| 206 | B | B |
| 207 | E | D |
| 208 | E | D |
| 209 | D | D |
| 210 | A | A |
| 211 | E | D |
| 212 | E | D |
| 213 | E | D |
| 214 | B | C |
| 215 | D | D |
| 216 | A | A |
| 217 | C | C |
| 218 | A | A |
| 219 | B | B |
| 220 | A | A |
| 221 | C | C |
| 222 | A | A |
| 223 | B | C |
| 224 | A | B |
| 225 | D | C |
| 226 | B | A |
| 227 | B | B |
| 228 | A | A |
| 229 | A | A |
| 230 | A | A |
| 231 | C | C |
| 232 | A | A |
| 233 | B | B |
| 234 | A | A |
| 235 | B | C |
| 236 | A | B |
| 237 | B | B |
| 238 | A | A |
| 239 | B | B |
| 240 | A | A |
| 241 | A | B |
| 242 | A | A |
| 243 | B | A |
| 244 | A | A |
| 245 | C | C |
| 246 | A | A |
| 247 | C | C |
| 248 | A | A |
| 249 | B | C |
| 250 | A | B |
| 251 | B | A |
| 252 | A | A |
| 253 | B | B |
| 254 | A | A |
| 255 | B | A |
| 256 | A | A |
| 257 | B | B |
| 258 | A | A |
| 259 | B | B |
| 260 | B | C |
| 261 | A | A |
| 262 | A | A |
| 263 | A | A |
| 264 | A | A |

### REFERENCES

1: Bachmaier, et. al. Nature (2000) 403:211-216
2: Chiang, et. al. Nature (2000) 403:216-220
3: Ronchi and Haas, Methods Mol Biol. (2012) 832: 197

1. A compound of formula (A-1) or (A-2): 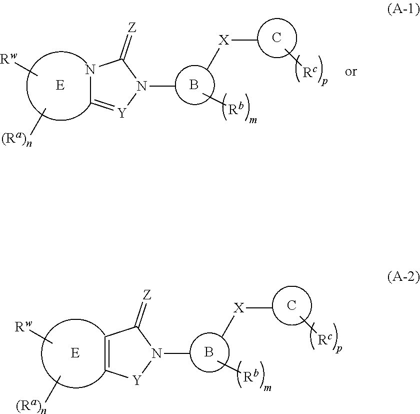
or pharmaceutically acceptable salts thereof,
wherein
Y is selected from the group ═C(H)—, ═C(R^{a})— or ═N—;
Z is ═O or ═S;
E is optionally substituted 5-6 membered heterocyclyl or optionally substituted phenyl;
B is optionally substituted phenyl, optionally substituted 8-10 membered bicyclyl, or optionally substituted 5-6 membered heteroaryl;
C is optionally substituted 5-6 membered heteroaryl;
X is an optionally substituted C₁-C₃ alkylene chain, wherein one or more methylene units is optionally and independently replaced by —N(H)—, —N(R¹)—, —O—, —S—, —SO—, —SO₂—, optionally substituted 3-6-membered carbocyclyl, and optionally substituted 3-6-membered heterocyclyl, wherein X is optionally substituted with an optionally substituted group selected from the group consisting of halogen, C₁-C₃ aliphatic, phenyl, 3-6-membered heteroaryl, 3-6-membered heterocyclyl, and —(CH₂)(3-6-membered carbocyclyl);
each R^{a} is independently selected from the group consisting of halogen, —CN, —OH, —OR¹, —NH₂, —NR¹R², —SH, —SR¹, —SF₅, —CO₂H, —CO₂R¹, —C(O)R¹, —CONH₂, —CONR¹R², —SO₂NH₂, —SO₂NR¹R², —SO₂OH, —SO₂OR¹, —S(O)R¹, —S(O)₂R¹, —S(O)(NH)R¹, —S(O)(NR¹)R¹, optionally substituted C₁-C₆ aliphatic, optionally substituted C₁-C₆ heteroalkyl, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S, wherein R^{a} is optionally substituted with 1-5 instances of R^{a1};
each R^{a1} is independently selected from the group consisting of halogen, —CN, —OH, —OR¹, —NH₂, —NR¹R², —SH, —SR¹, —SF₅, —CO₂H, —CO₂R¹, —CONH₂, —CONR¹R², —SO₂NH₂, —SO₂NR¹R², —SO₂OH, —SO₂OR¹, —S(O)R¹, —S(O)₂R¹, —S(O)(NH)R¹, —S(O)(NR¹)R¹, optionally substituted C₁-C₆ aliphatic, optionally substituted C₁-C₆ heteroalkyl, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S;
R^{w} is -L¹-A-L²-G;
L¹ is a bond or an optionally substituted C₁-C₃ alkylene chain;
L² is an optionally substituted C₁-C₁₂ alkylene chain, wherein 1-6 methylene units of L² are optionally and independently replaced by —C(O)—, —N(H)—, —N(R¹)—, —O—, —S—, —SO—, —SO₂—, optionally substituted 3-6-membered carbocyclyl, and optionally substituted 3-6-membered heterocyclyl, wherein X is optionally substituted with an optionally substituted group selected from the group consisting of halogen, C₁-C₃ aliphatic, phenyl, 3-6-membered heteroaryl, and 3-6-membered heterocyclyl;
A is a bivalent group selected from the group consisting of a bond, optionally substituted C₃-C₇ carbocyclylene, optionally substituted C₁-C₆ heteroalkylene, optionally substituted 3-6 membered heterocyclylene containing 1-4 heteroatoms each selected from the group consisting of N, O, and S, optionally substituted phenylene, and optionally substituted 5-6-membered heteroarylene containing 1-4 heteroatoms each selected from the group consisting of N, O and S, wherein A is optionally substituted with 1-5 instances of R^{a1};
G is selected from the group consisting of optionally substituted C₃-C₁₀ carbocylyl, optionally substituted C₁-C₆ heteroalkyl, optionally substituted 3-10 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S, wherein G is optionally substituted with 1-5 instances of R^{g1};
each R^{g1} is independently selected from the group consisting of halogen, —CN, —OH, —OR¹, —NH₂, —NR¹R², —SH, —SR¹, —SF₅, —CO₂H, —CO₂R¹, —CONH₂, —CONR¹R², —SO₂NH₂, —SO₂NR¹R², —SO₂OH, —SO₂OR¹, —S(O)R¹, —S(O)₂R¹, —S(O)(NH)R¹, —S(O)(NR¹)R¹, optionally substituted C₁-C₆ aliphatic, optionally substituted C₁-C₆ heteroalkyl, optionally substituted 3-10 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S;
each R^{b} is independently selected from the group consisting of, halogen, —CN, —OH, —OR¹, —NH₂, —NR¹R², —SH, —SR¹, —SF₅, —CO₂H, —CO₂R¹, —CONH₂, —CONR¹R², —SO₂NH₂, —SO₂NR¹R², —SO₂OH, —SO₂OR¹, —S(O)R¹, —S(O)₂R¹, —S(O)(NH)R¹, —S(O)(NR¹)R¹, optionally substituted C₁-C₆ aliphatic, optionally substituted C₁-C₆ heteroalkyl, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S;
each R^{c} is independently selected from the group consisting of hydrogen, optionally substituted C₁-C₆ aliphatic, OR¹, —NH₂, —NR¹R², optionally substituted phenyl, optionally substituted 3-6 membered heterocyclyl containing 1-4 heteroatoms each selected from the group consisting of N, O, and S, optionally substituted 5-6-membered heteroaryl containing 1-4 heteroatoms each selected from the group consisting of N, O and S, —C(O)R³, —CO₂R³, —C(O)NHR³, and —SO₂R³;
each R¹ is independently selected from the group consisting of optionally substituted C₁-C₆ aliphatic, optionally substituted phenyl, optionally substituted 3-6 membered heterocyclyl containing 1-4 heteroatoms each selected from the group consisting of N, O, and S, optionally substituted 5-6-membered heteroaryl containing 1-4 heteroatoms each selected from the group consisting of N, O and S, —C(O)R³, —CO₂R³, —C(O)NHR³, and —SO₂R³;
each R² is independently selected from the group consisting of hydrogen, optionally substituted C₁-C₆ aliphatic, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S;
or R¹ and R² are taken together with their intervening atom(s) to form a 3-8-membered heterocyclyl ring containing 1-3 heteroatoms selected from the group consisting of N, O, and S, or an optionally substituted 5-6-membered heteroaryl ring containing 1-4 heteroatoms selected from the group consisting of N, O, and S.
each R³ is independently selected from the group consisting of optionally substituted C₁-C₆ aliphatic, optionally substituted 3-6 membered heterocyclyl containing 1-4 heteroatoms each selected from the group consisting of N, O, and S, optionally substituted phenyl, optionally substituted 5-6-membered heteroaryl containing 1-4 heteroatoms each selected from the group consisting of N, O and S;
n is 0, 1, 2, 3, 4, or 5;
m is 0, 1, 2, 3, or 4; and
p is 0, 1, 2, 3, or 4.

2. The compound of claim 1, wherein C is selected from the group consisting of optionally substituted triazolyl, optionally substituted pyrazolyl, optionally substituted isoxazolyl, optionally substituted thiazolyl, optionally substituted thiadiazolyl, optionally substituted pyridinyl, optionally substituted pyrazinyl, optionally substituted pyrimidinyl, and optionally substituted pyridazinyl.

3. The compound of any of claims 1-2, wherein the compound is of Formula (B-1) or (B-2): 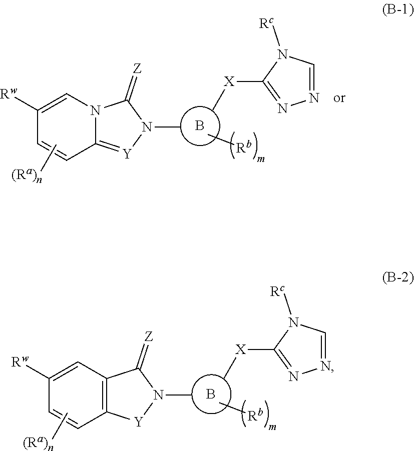
or pharmaceutically acceptable salts thereof.

4. The compound of any of claims 1-2, wherein the compound is of Formula (I-1 or I-2): 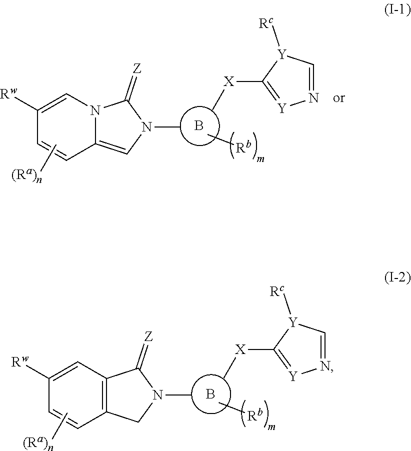
or pharmaceutically acceptable salts thereof.

5. The compound of any of claims 1-4, wherein the compound is of Formula (Ia) or (IIa): 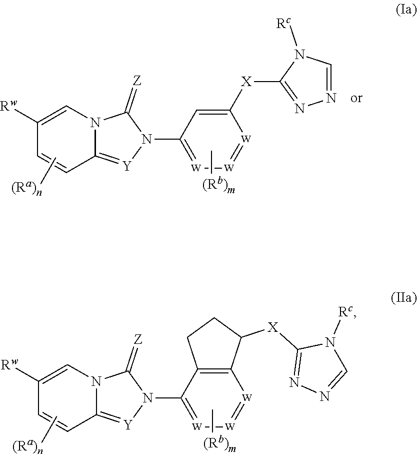
or a pharmaceutically acceptable salt thereof,
wherein each W is independently selected from N or C.

6. The compound of any of claims 1-5, wherein the compound is of formula (Ia1), (IIa1), (Ia1′), or (IIa1′): 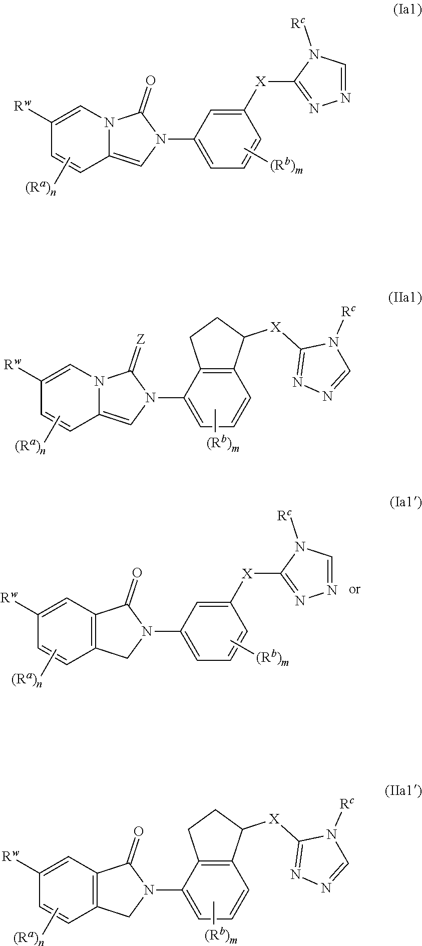
or a pharmaceutically acceptable salt thereof.

7. The compound of any of claims 1-5, wherein the compound is of Formula (Ia2), (Ia3), or (Ia4): 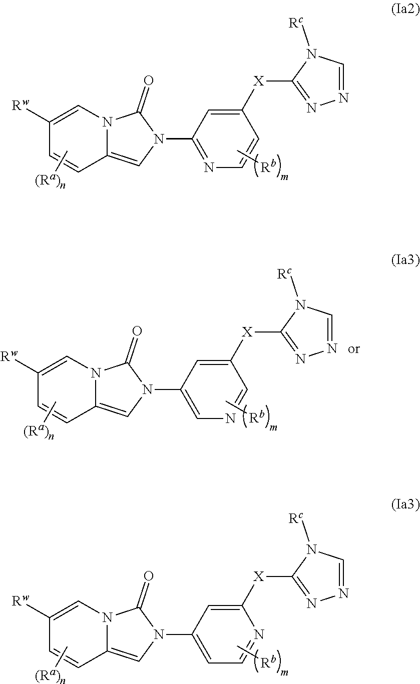
or a pharmaceutically acceptable salt thereof.

8. The compound of any of claims 1-5, wherein the compound is of formula (Ib1), (IIb1), (Ib2), or (IIb2): 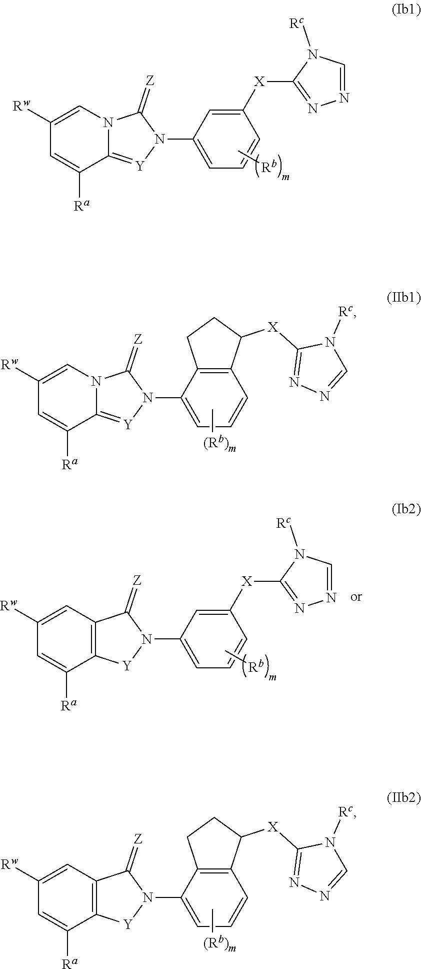
or pharmaceutically acceptable salts thereof,
wherein each W is independently selected from N or C.

9. The compound of any of claims 1-5, wherein the compound is of formula (Ic) or (IIc): 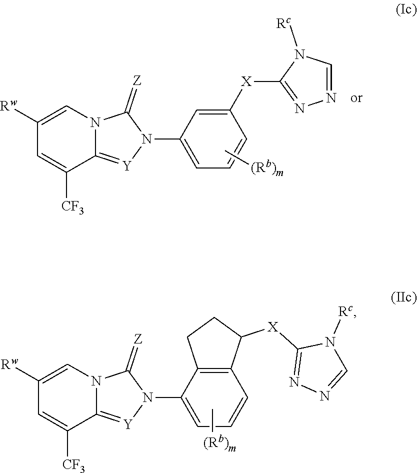
or pharmaceutically acceptable salts thereof.

10. The compound of any of claims 1-9, wherein R^{c} is optionally substituted C₁-C₃ aliphatic.

11. The compound of claim 10, wherein each R^{c} is independently selected from the group consisting of methyl, —CD₃, —CHF₂

12. The compound of claim 11, wherein R^{c} is methyl.

13. The compound of any of claims 1-12, wherein X is optionally substituted C₁-C₂ alkylene.

14. The compound of any of claims 1-12, wherein X is 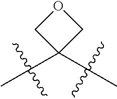 [SMILES: *C1(*)COC1]
or optionally substituted C₂ alkylene, wherein one methylene unit is replaced with

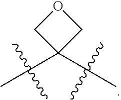 [SMILES: *C1(*)COC1]

15. The compound of any of claims 1-12, wherein X is selected from the group consisting of

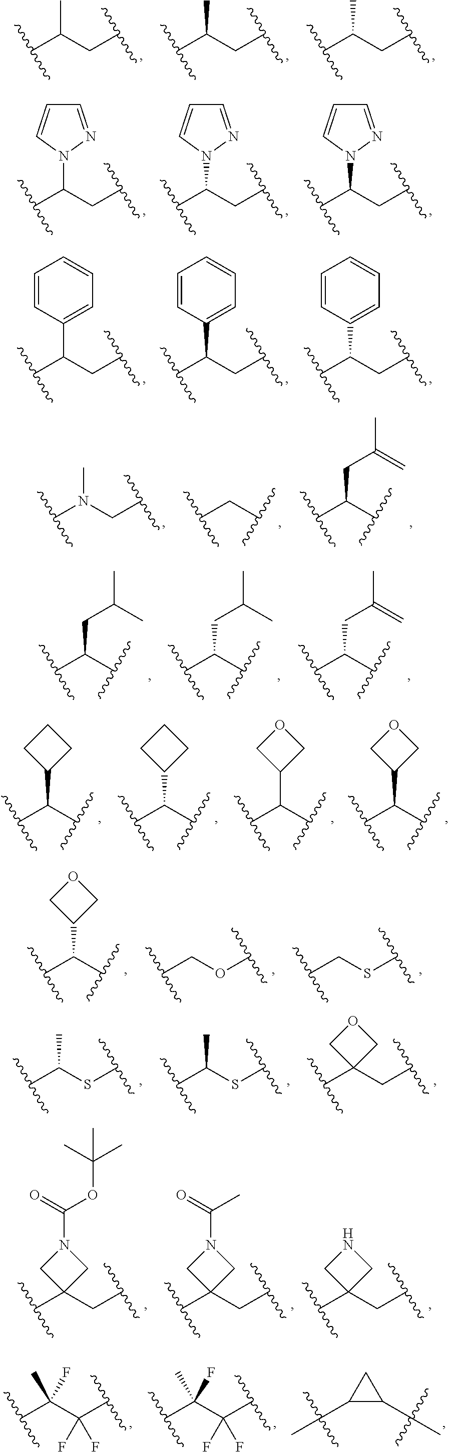

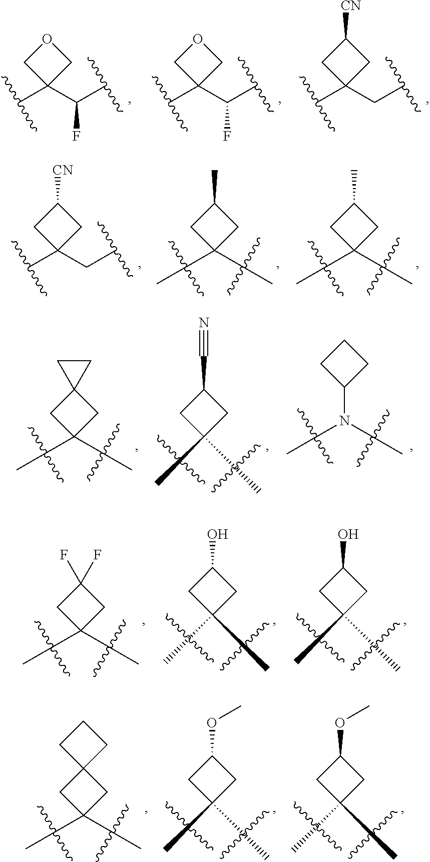

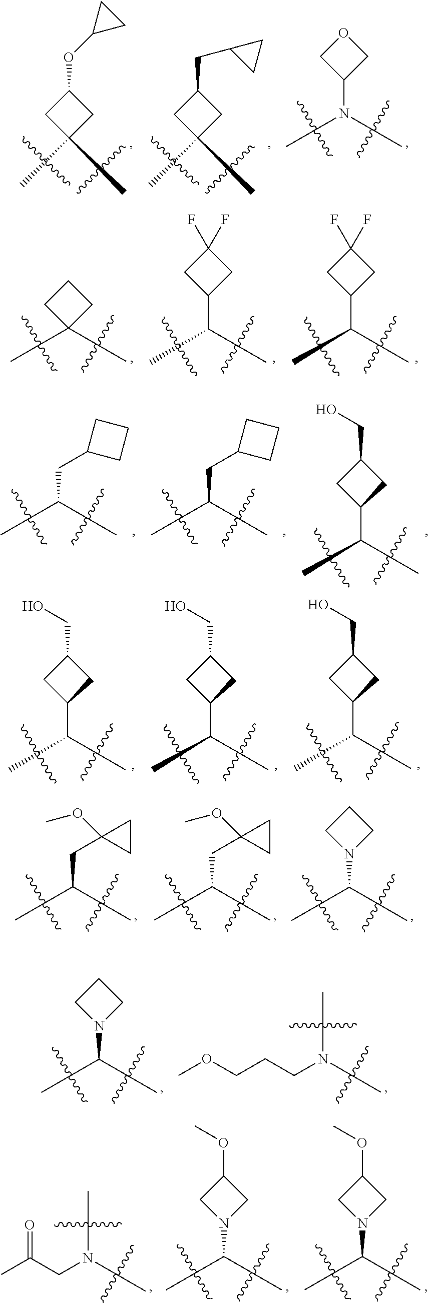

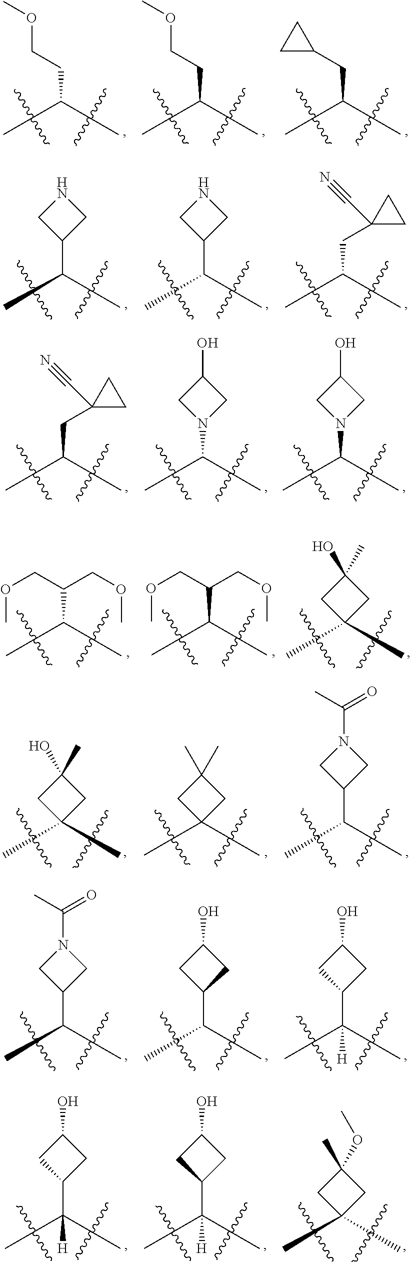

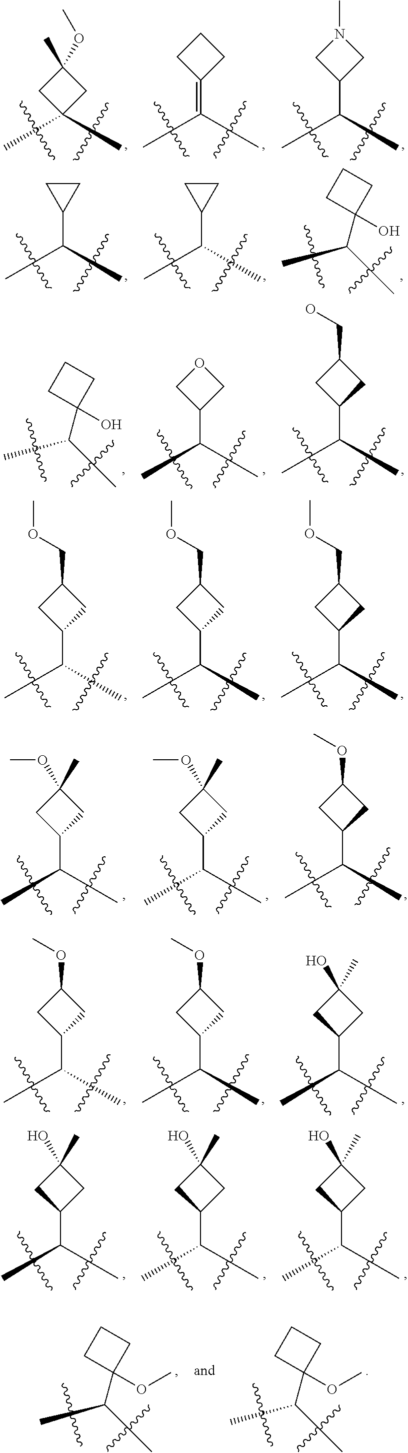

16. The compound of any of claims 1-15, wherein L¹ is —CH₂— or —CH(CH₃)—.

17. The compound of claim 16, wherein L¹ is —CH₂—.

18. The compound of any of claims 16-17, wherein A is optionally substituted 3-6 membered heterocyclylene containing 1-4 heteroatoms each selected from the group consisting of N, O, and S.

19. The compound of any of claims 16-17, wherein A is optionally substituted 6-membered heterocyclylene containing 1-4 heteroatoms each selected from the group consisting of N, O, and S.

20. The compound of any of claims 16-17, wherein A is optionally substituted 6-membered heterocyclylene selected from the group consisting of piperidinylene, piperazinylene

21. The compound of any of claims 16-17, wherein A is a bond.

22. The compound of any of claims 1-16, wherein R^{a} is selected from halogen, —CN, —C(O)R¹, —CO₂H, —CONR¹R², optionally substituted C₁-C₆ aliphatic, and optionally substituted C₁-C₆ heteroalkyl.

23. The compound of any of claims 1-16, wherein each R^{a} is independently selected from the group consisting of halogen, —CN, —CO₂H, —CHO, —CHF₂, —CF₃, —OMe, and —S(O)₂NHMe.

24. The compound of claim 16, wherein A is —CH₂— or —CH(CH₃)—.

25. The compound of any of claims 1-24, wherein L² is an optionally substituted C₁-C₁₂ alkylene chain, wherein 1-6 methylene units of L² are optionally and independently replaced by —N(H)—, —N(R¹)—, or —O—.

26. The compound of any of claims 1-24, wherein L² is an optionally substituted C₁-C₁₂ alkylene chain, wherein 1-6 methylene units of L² are optionally and independently replaced by —N(H)—, —N(R¹)—, —O—, or optionally substituted phenylene.

27. The compound of any of claims 1-24, wherein L² is an optionally substituted C₁-C₁₂ alkylene chain, wherein 1-6 methylene units of L² are optionally and independently replaced by —N(H)—, —N(R¹)—, —O—, or optionally substituted 3-8-membered heterocyclyl.

28. The compound of any of claims 1-24, wherein L² is an optionally substituted C₁-C₁₂ alkylene chain, wherein 1-6 methylene units of L² are optionally and independently replaced by —N(H)—, —N(R¹)—, —O—, or optionally substituted 6-membered heterocyclyl.

29. The compound of any of claims 1-24, wherein L² is an optionally substituted C₁-C₁₂ alkylene chain, wherein 1-6 methylene units of L² are optionally and independently replaced by —O—, or optionally substituted 3-6-membered heterocyclyl.

30. The compound of any of claims 1-24, wherein L² is an optionally substituted C₁-C₁₂ alkylene chain, wherein 1-6 methylene units of L² are optionally and independently replaced by —O—, or optionally substituted 6-membered heterocyclyl.

31. The compound of any of claims 1-24, wherein L² is an optionally substituted C₁-C₁₂ alkylene chain, wherein 1-6 methylene units of L² are optionally and independently replaced by —N(H)—, —N(R¹)—, or optionally substituted 3-8-membered heterocyclyl.

32. The compound of any of claims 1-24, wherein L² is an optionally substituted C₁-C₁₂alkylene chain, wherein 1-6 methylene units of L² are optionally and independently replaced by —N(H)—, —N(R¹)—, or optionally substituted 6-membered heterocyclyl.

33. The compound of any of claims 1-24, wherein L² is an optionally substituted C₁-C₁₂alkylene chain, wherein 1-6 methylene units of L² are optionally and independently replaced by —N(H)—, —N(R¹)—, —O—, optionally substituted 6-membered heterocyclyl, or optionally substituted phenylene.

34. The compound of any of claims 1-24, wherein L² is an optionally substituted C₁-C₁₂ alkylene chain, wherein 1-6 methylene units of L² are optionally and independently replaced by —N(H)—, —N(R¹)—, —O—,

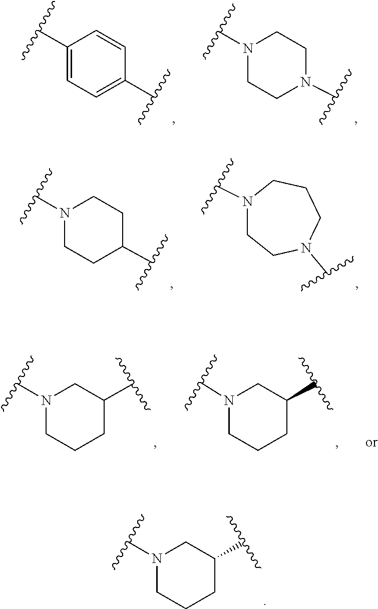

35. The compound of any of claims 1-25, each R^{g1} is independently halogen or optionally substituted 6-membered heterocyclyl.

36. The compound of claim 35, wherein R^{g1} is glutarimidyl.

37. The compound of any of claims 1-36, wherein G is optionally substituted 9-membered heterocyclyl.

38. The compound of claim 37, wherein G is isoindoline or phthalimide.

39. The compound of claim 38, wherein G is selected from the group consisting of

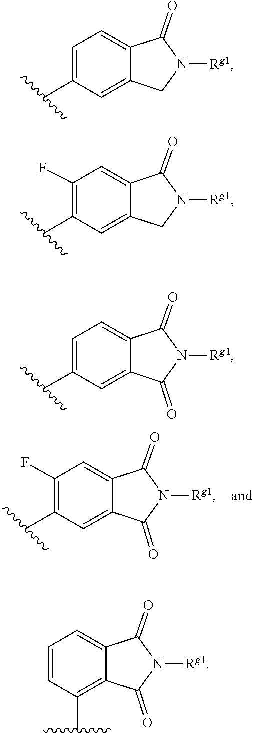

40. A compound selected from those listed in Table 1.

41. A pharmaceutical composition comprising a compound of any of claims 1-40 and a pharmaceutically acceptable adjuvant or carrier.

42. A method of treating a disease or condition associated with cell proliferation comprising administering a therapeutically effective amount of a compound of any of claims 1-40 or a pharmaceutical composition of claim 41 to a subject in need thereof.

43. The method of claim 42, wherein the disease or condition associated with cell proliferation is hyperplasia or cancer.

44. The method of claim 43, wherein cancer is a hematologic cancer.

45. The method of claim 44, wherein the hematologic cancer is selected from a group consisting of lymphoma, leukemia, and myeloma.

46. The method of claim 45, wherein cancer is a non-hematologic cancer.

47. The method of claim 46, wherein the non-hematologic cancer is a sarcoma or a carcinoma.

48. The method of any one of claims 42-47, wherein the subject has one or more of increased T-cell activation, increased T-cell proliferation, decreased T-cell exhaustion, decreased T-cell anergy and decreased T-cell tolerance after administration of compound of any of claims 1-15 or a pharmaceutical composition of claim 16.

49. The method of claim 48, wherein increased T-cell activation comprises increased production of a cytokines.

50. The method of claims 42-47, wherein the subject has increased NK-cell activation.

51. The method of 50, the increased NK-cell activation comprises increased production of cytokines.
